(19) United States
(12) Patent Application Publication
(10) Pub. No.: US 2025/0108123 A1
(43) Pub. Date: Apr. 3, 2025

(54) COMPOUND-LINKER CONSTRUCTS COMPRISING NOVEL COMPOUNDS USEFUL AS STING AGONISTS AND USES THEREOF

(71) Applicant: Jacobio Pharmaceuticals Co., Ltd., Beijing (CN)

(72) Inventors: Yang YE, Beijing (CN); Xiuwei LI, Beijing (CN); Guiqun YANG, Beijing (CN); Hongling WANG, Beijing (CN); Xiong FANG, Beijing (CN); Yankai CUI, Beijing (CN); Fashun YAN, Beijing (CN); Binhua SONG, Beijing (CN)

(21) Appl. No.: 18/720,167

(22) PCT Filed: Dec. 16, 2022
(86) PCT No.: PCT/CN2022/139560

§ 371 Date: Jun. 14, 2024

(30) Foreign Application Priority Data
Dec. 17, 2021 (WO) PCT/CN2021/139176
Jul. 1, 2022 (WO) PCT/CN2022/103381
Aug. 25, 2022 (WO) PCT/CN2022/114795
Sep. 16, 2022 (WO) PCT/CN2022/119305
Sep. 28, 2022 (WO) PCT/CN2022/122093
Oct. 28, 2022 (WO) PCT/CN2022/128306

(51) Int. Cl. A61K 47/68; A61K 39/00; A61K 47/54; C07K 16/28

(52) U.S. Cl. CPC A61K 47/6851; A61K 47/54; A61K 47/6803; C07K 16/2863; A61K 2039/505

(57) ABSTRACT

Provided herein are compound-linker constructs and antibody-drug-conjugates of compounds of formula (Y-1), (Y-2), (Y-3), (A), (B), (C), I, II, III, IV or V that are useful as modulators of STING (Stimulator of Interferon Genes). Also provided are synthesis, compositions and uses of such compound-linker constructs and antibody-drug-conjugates.

### TECHNICAL FIELD

The present disclosure relates to functionalized compounds of formula (Y-1), (Y-2), (Y-3), (A), (B), (C), I, II, III, IV or V in the form of compound-linker constructs and conjugates that are useful as modulators of STING. The present disclosure further relates to compositions comprising such conjugates or constructs, processes for the synthesis thereof, and uses thereof.

### BACKGROUND ART

The immune system has evolved to recognize and neutralize different types of threats in order to maintain the homeostasis of the host, and it is generally broken down into two arms: adaptive and innate. The adaptive immune system is specialized to recognize as foreign those antigens not naturally expressed in the host and to mount an anti-antigen response through the coordinated actions of many leukocyte subsets. The hallmark of adaptive immune responses is their ability to provide “memory” or long-lasting immunity against the encountered antigen. While this specific and long-lasting effect is critical to host health and survival, the adaptive immune response requires time to generate a full-blown response.

The innate immune system compensates for this time delay and is specialized to act quickly against different insults or danger signals. It provides the first line of defense against bacteria, viruses, parasites and other infectious threats, but it also responds strongly to certain danger signals associated with cellular or tissue damage. The innate immune system has no antigen specificity but does respond to a variety of effector mechanisms. Opsonization, phagocytosis, activation of the complement system, and production of soluble bioactive molecules such as cytokines or chemokines are all mechanisms by which the innate immune system mediates its response. By responding to these damage-associated molecular patterns (DAMPs) or pathogen-associated molecular patterns (PAMPs) described above, the innate immune system is able to provide broad protection against a wide range of threats to the host.

Free cytosolic DNA and RNA are among these PAMPs and DAMPs. It has recently been demonstrated that the main sensor for cytosolic DNA is cGAS (cyclic GMP-AMP synthase). Upon recognition of cytosolic DNA, cGAS catalyzes the generation of the cyclic-dinucleotide 2′3′-cGAMP, an atypical second messenger that strongly binds to the ER-transmembrane adaptor protein STING. A conformational change is undergone by cGAMP-bound STING, which translocates to a perinuclear compartment and induces the activation of critical transcription factors IRF-3 and NF-κB. This leads to a strong induction of type I interferons and production of pro-inflammatory cytokines such as IL-6, TNF-α and IFN-γ.

The importance of type I interferons and pro-inflammatory cytokines on various cells of the immune system has been very well established. In particular, these molecules strongly potentiate T-cell activation by enhancing the ability of dendritic cells and macrophages to uptake, process, present and cross-present antigens to T-cells. The T-cell stimulatory capacity of these antigen-presenting cells is augmented by the up-regulation of critical co-stimulatory molecules, such as CD80 or CD86. Finally, type I interferons can rapidly engage their cognate receptors and trigger the activation of interferon-responsive genes that can significantly contribute to adaptive immune cell activation.

From a therapeutic perspective, type I interferons are shown to have antiviral activities by directly inhibiting human hepatitis B virus and hepatitis C virus replication, and by stimulating immune responses to virally infected cells. Compounds that can induce type I interferon production are used in vaccines, where they act as adjuvants, enhancing specific immune responses to antigens and minimizing side effects by reducing dosage and broadening the immune response.

In addition, interferons, and compounds that can induce interferon production, have potential use in the treatment of human cancers. Such molecules are potentially useful as anti-cancer agents with multiple pathways of activity. Interferons can inhibit human tumor cell proliferation directly and may be synergistic with various approved chemotherapeutic agents. Type I interferons can significantly enhance anti-tumor immune responses by inducing activation of both the adaptive and innate immune cells. Finally, tumor invasiveness may be inhibited by interferons by modulating enzyme expression related to tissue remodeling.

In view of the potential of type I interferons and type I interferon-inducing compounds as anti-viral and anti-cancer agents, there remains a need for new agents that can induce potent type I interferon production. With the growing body of data demonstrating that the cGAS-STING cytosolic DNA sensory pathway has a significant capacity to induce type Iinterferons, the development of STING activating agents is rapidly taking an important place in today's anti-tumor therapy landscape.

The covalent attachment of small molecule compounds to a linker and optionally additionally to a targeting moiety can mask the compound from the host's immune system (reducing immuno-genicity and antigenicity), and increase its hydrodynamic size (size in solution), which prolongs its circulation time by reducing renal clearance. Furthermore, the water solubility of the compound can be positively influenced by the use of a suitable linker with hydrophilic groups.

Conjugates of small molecule compounds with targeting moieties, in particular antibody-drug conjugates (ADC), combine the targeting capabilities of, e.g., a monoclonal antibody, with the pharmacological activity of the attached compounds (also referred to as payloads). In particular, the targeting moiety may specifically target a certain tumor antigen (e.g., a protein that, ideally, is only to be found in or on tumor cells) or immune cells antigen and attach itself to the antigens on the surface of cancerous cells or immune cells. The biochemical reaction between the targeting moiety, preferably an antibody, and the target protein (antigen) can trigger a signal in the tu-mor cell or immune cells, which then absorbs or internalizes the antibody together with the linked compound (payload). After the ADC is internalized (endocytosis), the linked compound will exhibit its pharmacological activity within the cell. This targeting limits side effects and gives a wider therapeutic window than other chemotherapeutic agents. In some cases, the payload may be sufficiently membrane-permeable to diffuse out of the cell and act in bystander cells. In another approach, a non-internalising mechanism of action is also possible. In this case, linker cleavage and payload release occur in the extracellular tumor microenvironment. Thus, ADC endocytosis is not required and non-internalising antigens may be selected as targets.

Overall, ADCs therefore aim to combine the favorable aspects of systemic administration of small molecular weight active compounds with targeted delivery via e.g., monoclonal antibodies therapies, thereby creating highly active and selective therapeutics with long plasma half-lives. Linkers in connection with ADCs link the small molecule compound (payload) with the targeting moiety, e.g., the antibody. ADC linkers can be classified as “cleavable” or “non-cleavable”, with cleavable linkers being the preferred choice (J. D. Bargh et al., Chem. Soc. Rev., 2019, DOI: 10.1039/c8cs00676h).

In view of the above, drugs modulating STING are useful for treating one or more diseases selected from the group consisting of inflammatory, allergic, and autoimmune diseases, infectious diseases, cancer, pre-cancerous syndromes, and/or as immunogenic composition or vaccine adjuvants. Of particular relevance is the immunotherapy of cancer and viral infections, in particular prostate cancer, renal carcinoma, melanoma, pancreatic cancer, cervical cancer, ovarian cancer, colon cancer, head and neck cancer, lung cancer, fibrosarcoma, and breast cancer. Furthermore, activation of local immune response to the lesions is considered to be preferably parenteral or non-parenteral therapeutic approach.

Accordingly, there is a need for drugs modulating the activity of STING, and accordingly, provide a therapeutic impact in the treatment of diseases, in which the modulation of STING is beneficial. And it is an object of the present disclosure to provide functionalized compounds, e.g., in the form of compound-linker constructs or conjugates with targeting moieties such as antibodies.

### SUMMARY OF INVENTION

The present disclosure relates to compounds of general formula (Y-1), (Y-2), (Y-3), (A), (B), (C), I, II, III, IV or V and pharmaceutically acceptable salts thereof, which modulate STING. The present disclosure comprises functionalized compounds of formula (Y-1), (Y-2), (Y-3), (A), (B), (C), I, II, III, IV or V in the form of compound-linker constructs and conjugates that are useful as modulators of STING. These compounds, compound constructs, conjugates as well as their pharmaceutical composition may be useful as agents to induce immune responses, to induce STING-dependent type I interferon production, and/or to treat a cell proliferation disorder.

In a first aspect, the present disclosure relates to a compound-linker construct useful for conjugating with a T, wherein the compound-linker construct is of formula S3:

T^{B}ₘ-D-L¹ (S3)
or a pharmaceutically acceptable salt or solvate thereof, wherein:
D is a STING agonist moiety,
L¹ is a linker;
T^{B} is a hydrophilic group; and
m is O or 1.

In some embodiments, T^{B} is a water-soluble and substantially non-antigenic polymer. Examples of the hydrophilic group, include, but are not limited to, polyalcohols, polyethers, polyanions, polycations, polyphosphoric acids, polyamines, polysaccharides, polyhydroxy compounds, polylysines, and derivatives thereof.

In some embodiments, T^{B} comprises a plurality of hydroxyl (“—OH”) groups, such as moieties that incorporate monosaccharides, oligosaccharides, polysaccharides, and the like.

In some embodiments, T^{B} comprises a plurality of —(CR₅₈OH)— groups, wherein R₅₈ is —H or C₁₋₈ alkyl.

In some embodiments, T^{B} is

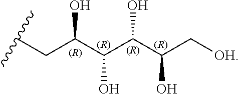

In some embodiments, T^{B} is PEGₙ, wherein n is 1-16, preferably 1-8.

In some embodiments, T^{B} is PEGₙ-(CR₅₈OH)ⱼ, wherein: n is 1-16, preferably 1-8; R₅₈ is —H or C₁₋₈ alkyl; and j is 1 or 2.

In a second aspect, the present disclosure relates to a compound-linker construct useful for conjugating with a T, wherein the compound-linker construct is of formula S2:

D-L¹ (S2)
or a pharmaceutically acceptable salt or solvate thereof, wherein:
D is a STING agonist moiety, and
L¹ is a linker.

In some embodiments, the compound-linker construct is of fomula S2a:

D-H^{1A}-L^{C}-H^{1B} (S2a)
or a pharmaceutically acceptable salt or solvate thereof.

In some embodiments, the compound-linker construct is useful for conjugating with a T, and T is a targeting moiety.

In some embodiments, the compound-linker construct is of formula S2b:

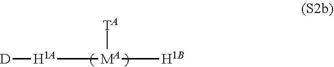

or a pharmaceutically acceptable salt or solvate thereof.

In a third aspect, the present disclosure relates to a conjugate of formula SO:

[T^{B}ₘ-D-L¹]_{d5}-T (S0)
or a pharmaceutically acceptable salt or solvate thereof, wherein:
D is a STING agonist moiety,
L¹ is a linker;
T is a targeting moiety;
T^{B} is a hydrophilic group which is defined as herein;
m is O or 1; and
d5 is an integer from 1 to 20;
wherein T^{Bm}-D-L¹ is a compound-linker construct, and a covalent bond between the compound-linker construct and the targeting moiety T is established by the reaction of a functional group of the targeting moiety T with a functional group handle of the linker L¹ of the compound-linker construct.

In a fourth aspect, the present disclosure relates to a conjugate of formula S1:

[D-L¹]_{d5}-T (S1)
or a pharmaceutically acceptable salt or solvate thereof, wherein:
D is a STING agonist moiety,
L¹ is a linker;
T is a targeting moiety; and
d5 is an integer from 1 to 20;
wherein D-L¹ is a compound-linker construct, and a covalent bond between the compound-linker construct and the targeting moiety T is established by the reaction of a functional group of the targeting moiety T with a functional group handle of the linker L¹ of the compound-linker construct.

In some embodiments, the conjugate is of formula Sla:

[D-H^{1A}-L^{C}-H^{1B}]_{d5}-T (S1a)
or a pharmaceutically acceptable salt or solvate thereof.

In some embodiments, the conjugate is of formula S1b:

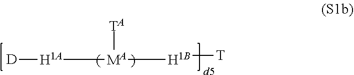

or a pharmaceutically acceptable salt or solvate thereof.

It is understood that, for a compound-linker construct of any one of formula S3, S2, S2a, S2b, or a conjugate of any one of formula S0, S1, S1a, S1b, or a pharmaceutically acceptable salt or solvate thereof, variables T, L¹, H^{1A}, T^{A}, M^{A}, H^{1B}, D, and d5 can each be, where applicable, selected from the groups described herein, and any group described herein for any of variables T, L¹, H^{1A}, T^{A} M^{A}, H^{1B}, D, and d5 can be combined, where applicable, with any group described herein for one or more of the remainder of variables T, L¹, H^{1A}, T^{A} MA, H^{1B}, D, and d5.

In some embodiments, the STING agonist moiety [D] is a compound of Formula Y-1, Y-2, Y-3, or an ester, stereoisomer, tautomer, isotopic derivative, prodrug or pharmaceutically acceptable salt:

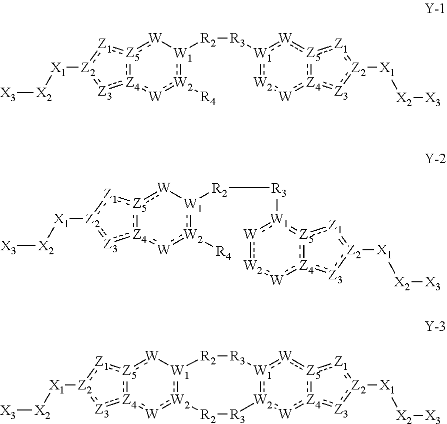

wherein,
[symbol] represents a single bond or a double bond;
each W is independently selected from CR₁, C(R₁)₂, N, NR₁, O or S;
each W₁ is independently selected from C, CR₁, or N;
each W₂ is independently selected from C, CR₁, or N;
each Z₁ is independently selected from CR₁, C(R₁)₂, N, NR₁, O or S;
each Z₂ is independently selected from CR₁, C or N;
each Z₃ is independently selected from CR₁, C(R₁)₂, N, NR₁, O or S;
each Z₄ is independently selected from C, CR₁ or N;
each Z₅ is independently selected from C, CR₁ or N;
each R₁ is independently selected from H, deuterium, halogen, OR⁶, N(R⁶)₂, COOR⁶, or C(O)N(R⁶)₂, CN or C₁-C₆ alkyl; wherein the C₁-C₆ alkyl is optionally substituted with one or more deuterium, halogen, OR⁶, N(R⁶)₂, COOR⁶, or C(O)N(R⁶)₂;
R₂ and R₃ are independently selected from the group consisting of O—(C₁-C₄ alkylene or haloalkylene), C₁-C₅ alkylene or haloalkylene, N(R⁶)—(C₁-C₄ alkylene or haloalkylene), -Tₐ-C₁-C₆ alkyl-T_{b}-, -Tₐ-N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}-, -Tₐ-C2-C6alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃-C₁₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ- (C₃-C₁₂cycloalkyl)-T_{b}-, -Tₐ- (C₆-C₁₂ aryl)-T_{b}-, -Tₐ-(3- to 12-membered heterocycloalkyl)-T_{b}-, or -Tₐ-(5to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs;
PEGₙ is (—OCH₂CH₂—)ₙ, n=1-16, preferably 1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O— (C₁-C₆ alkyl)-O—; wherein the C₁-C₆ alkyl is optionally substituted with one or more halogen; and
each Rs independently is H, deuterium or C₁-C₆ alkyl optionally substituted with one or more halogen;
each R⁴ is independently selected from the group consisting of H, deuterium, halogen, CN, OR⁶, N(R⁶)₂, COOR⁶, C(O)N(R⁶)₂, SO₂R⁶, C₁-C₆ alkyl, C₁-C₆ haloalkyl, C₁-C₆ alkyl substituted by OR⁶, C₂-C₆ alkenyl, C₂-C₆ haloalkenyl, C₂-C₆ alkenyl substituted by OR⁶, C₂-C₆ alkynyl, C₂-C₆ haloalkynyl, C₂-C₆ alkynyl substituted by OR⁶, C₃-C₆ cycloalkyl, and a 3- to 6-membered heterocyclic ring including 1 to 2 ring members selected from the group consisting of O, S, and N(R⁶);
each R⁶ is independently selected from the group consisting of —H, deuterium, halogen, —NH₂, —CN, —OH, —N₃, —NO₂, carboxyl, C₁₋₆alkyl, C₁₋₆alkoxy, C₂₋₆alkenyl, C₂₋₆alkynyl, —C₆₋₁₀ aryl, —C₅₋₁₀heteroaryl, C₃₋₁₀heterocyclic ring or C₃₋₁₀carbocyclic ring; and each of which is independently optionally substituted with deuterium, halogen, —NH₂, —CN, —OH, —NO₂, carbonyl, ═O, oxo, carboxyl, C₁₋₆alkoxy, or C₁₋₆alkyl; and each of the heteroaryl and heterocyclic ring contains at least one heteroatoms selected from N, O or S;
each X₁ is independently selected from the group consisting of C═O, —CH₂—, —CHF—, and —CF₂—;
each X₂ is independently selected from (C(R⁸)₂)₍₁₋₃₎, NR⁸(C(R⁸)₂)₍₁₋₃₎, —NH(C(R⁸)₂)₍₁₋₃₎, —N(C₁₋₆alkyl)(C(R⁸)₂)₍₁₋₃₎ or —N(haloC₁₋₆alkyl) (C(R⁸)₂)₍₁₋₃₎; wherein each R⁸ is independently selected from the group consisting of H, deuterium, halogen, C₁-C₆ alkyl, CN, OR⁶, N(R⁶)₂, C₁-C₆ haloalkyl, C₃-C₆ cycloalkyl, C₁-C₆ alkyl substituted by OR⁶, and C₁-C₆ alkyl substituted by N(R⁶)₂; optionally 2 R⁸ on different carbon atoms may be taken together, along with the atoms to which they are attached, to form a 3- to 6-membered fused ring; and optionally 2 R⁸ on a single carbon atom may be taken together, along with the atom to which they are attached, to form a 3- to 6-membered spirocycle;
each X₃ is independently selected from the group consisting of H, CN, COOR⁶, C(O)SR⁶, C(S)OR⁶, SO₂R⁶, C(O)N(R⁹)₂,

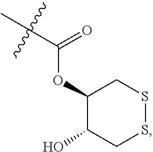 [SMILES: *C(=O)O[C@H]1CSSC[C@@H]1O]

OR⁶, SR⁶, N(R⁶)₂, OCOR⁶, NR⁶COR⁶, C₁₋₆alkyl, C₁₋₆alkoxy, C₂₋₆alkenyl, C₂₋₆alkynyl, —C₆₋₁₀aryl, —C₅₋₁₀heteroaryl, C₃₋₁₀heterocyclic ring or C₃₋₁₀carbocyclic ring; and each R⁹ is independently selected from H, deuterium, COOR⁶, SO₂R⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆alkyl), S(C₆-C₁₀ aryl), S(═O)₂R⁶, S(═O)₂O R⁶, P(═O)(R⁶)₂, C₁-C₆alkyl, C₂-C₆ alkenyl, C₂-C₆ alkynyl, C₃-C₈ cycloalkyl, C₆-C₁₀aryl, 3-8 membered heterocycloalkyl, or 3-10 membered heteroaryl.

In some embodiment of formula Y-1, Y-2 or Y-3,

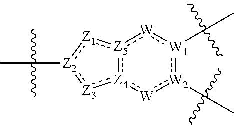

is independently selected from

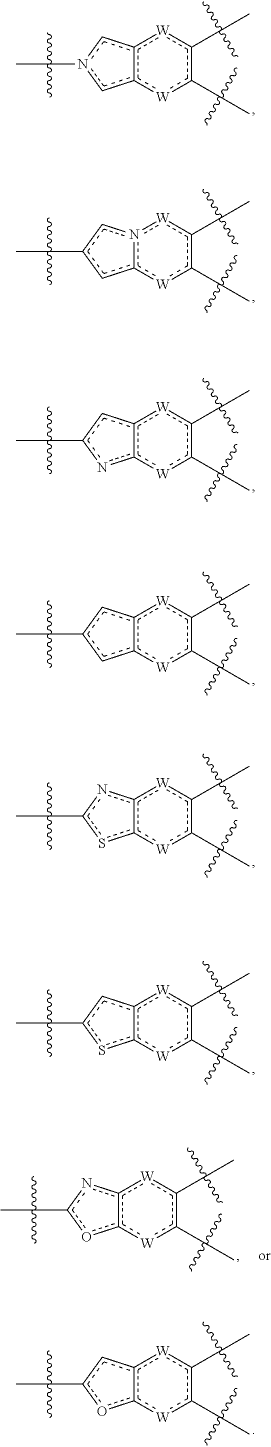

In some embodiment of formula Y-1, Y-2 or Y-3

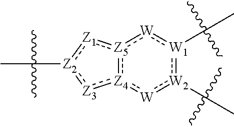

is independently selected from

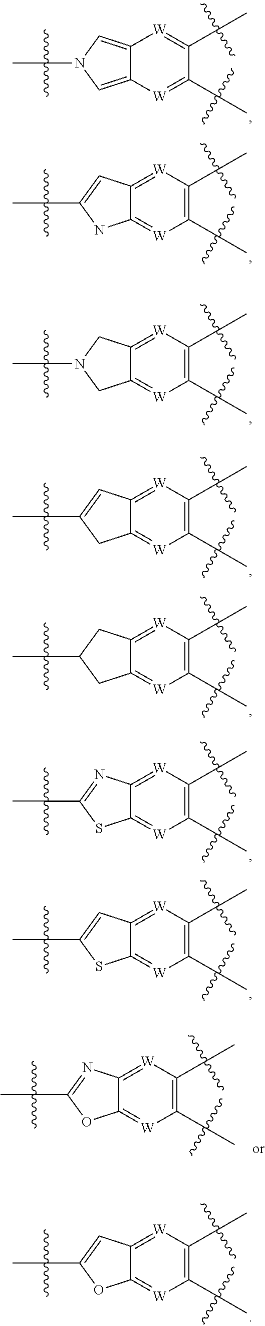

In some embodiment of formula Y-2,

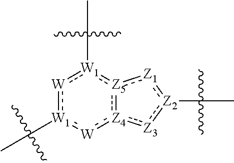

is independently selected from

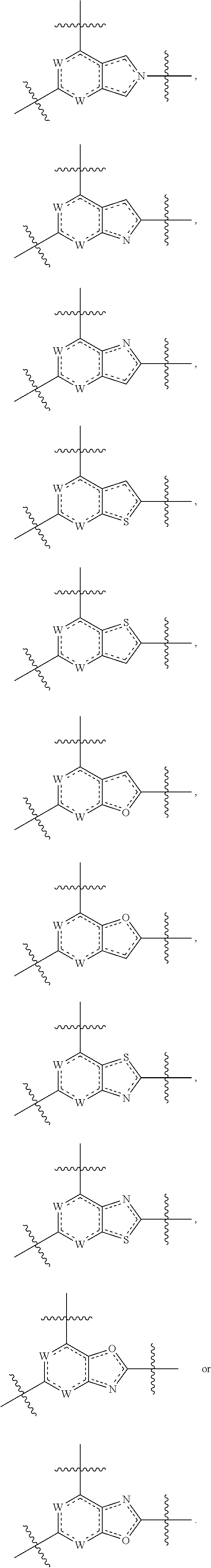

In some embodiment of formula Y-2,

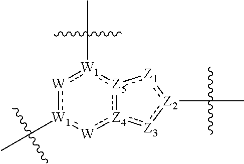

is independently selected from

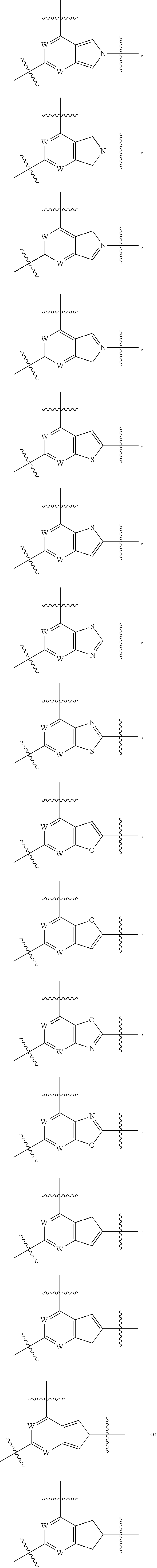

In some embodiments, the STING agonist moiety [D] is a compound of Formula (a), (b), (c), or a

pharmaceutically acceptable salt:

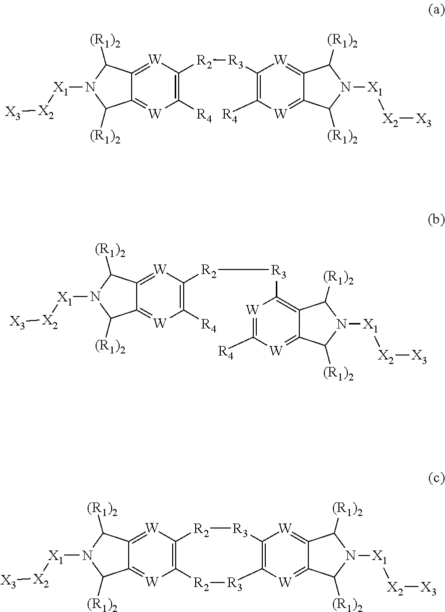

wherein,
each W is independently selected from CR₁ or N;
each R₁ is independently selected from H, deuterium, halogen, OR⁶, N(R⁶)₂, COOR⁶, or C(O)N(R⁶)₂, CN or C₁-C₆ alkyl; wherein the C₁-C₆ alkyl is optionally substituted with one or more deuterium, halogen, OR⁶, N(R⁶)₂, COOR⁶, or C(O)N(R⁶)₂;
R₂ and R₃ are independently selected from the group consisting of O—(C₁-C₄ alkylene or haloalkylene), C₁-C₅ alkylene or haloalkylene, N(R⁶)—(C₁-C₄ alkylene or haloalkylene), -Tₐ-C₁-C₆ alkyl-T_{b}-, -Tₐ- N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}-, - Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃-C₁₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ- (C₃-C₁₂cycloalkyl)-T_{b}-, -Tₐ- (C₆-C₁₂ aryl)-T_{b}-, -Tₐ-(3- to 12-membered heterocycloalkyl)-T_{b}-, or -Tₐ-(5to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O— (C₁-C₆ alkyl)-O—; wherein the C₁-C₆ alkyl is optionally substituted with one or more halogen; and
each Rs independently is H, deuterium or C₁-C₆ alkyl optionally substituted with one or more halogen;
each R⁴ is independently selected from the group consisting of H, deuterium, halogen, CN, OR⁶, N(R⁶)₂, COOR⁶, C(O)N(R⁶)₂, SO₂R⁶, C₁-C₆ alkyl, C₁-C₆ haloalkyl, C₁-C₆ alkyl substituted by OR⁶, C₂-C₆ alkenyl, C₂-C₆ haloalkenyl, C₂-C₆ alkenyl substituted by OR⁶, C₂-C₆ alkynyl, C₂-C₆ haloalkynyl, C₂-C₆ alkynyl substituted by OR⁶, C₃-C₆ cycloalkyl, and a 3- to 6-membered heterocyclic ring including 1 to 2 ring members selected from the group consisting of O, S, and N(R⁶);
each R⁶ is independently selected from the group consisting of —H, deuterium, halogen, —NH₂, —CN, —OH, —N₃, —NO₂, carboxyl, C₁₋₆alkyl, C₁₋₆alkoxy, C₂₋₆alkenyl, C₂₋₆alkynyl, —C₆₋₁₀aryl, —C₅₋₁₀heteroaryl, C₃₋₁₀heterocyclic ring or C₃₋₁₀carbocyclic ring; and each of which is independently optionally substituted with deuterium, halogen, —NH₂, —CN, —OH, —NO₂, carbonyl, ═O, oxo, carboxyl, C₁₋₆alkoxy, or C₁-C₆alkyl; and each of the heteroaryl and heterocyclic ring contains at least one heteroatoms selected from N, O or S;
each X₁ is independently selected from the group consisting of C═O, —CH₂—, —CHF—, and —CF₂—;
each X₂ is independently selected from (C(R⁸)₂)₍₁₋₃₎, NR⁸(C(R⁸)₂)₍₁₋₃₎, —NH(C(R⁸)₂)₍₁₋₃₎, —N(C₁₋₆alkyl)(C(R⁸)₂)₍₁₋₃₎ or —N(haloC₁₋₆alkyl) (C(R⁸)₂)₍₁₋₃₎; wherein each R⁸ is independently selected from the group consisting of H, deuterium, halogen, C₁-C₆ alkyl, CN, OR⁶, N(R⁶)₂, C₁-C₆ haloalkyl, C₃-C₆ cycloalkyl, C₁-C₆ alkyl substituted by OR⁶, and C₁-C₆ alkyl substituted by N(R⁶)₂; optionally 2 R⁸ on different carbon atoms may be taken together, along with the atoms to which they are attached, to form a 3- to 6-membered fused ring; and optionally 2 R⁸ on a single carbon atom may be taken together, along with the atom to which they are attached, to form a 3- to 6-membered spirocycle;
each X₃ is independently selected from the group consisting of COOR⁶, C(O)SR⁶, C(S)OR⁶, SO₂R⁶, C(O)N(R⁹)₂,

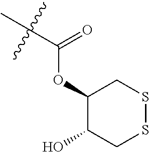 [SMILES: *C(=O)O[C@H]1CSSC[C@@H]1O]

and CN; and each R⁹ is independently selected from H, deuterium, COOR⁶, SO₂R⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆alkyl), S(C₆-C₁₀ aryl), S(═O)₂R⁶, S(═O)₂OR⁶, P(═O)(R⁶)₂, C₁-C₆alkyl, C₂-C₆ alkenyl, C₂-C₆ alkynyl, C₃-C₅ cycloalkyl, C₆-C₁₀aryl, 3-8 membered heterocycloalkyl, or 3-10 membered heteroaryl.

In some embodiments, the STING agonist moiety [D] is a compound of Formula (A), (B), (C), or a pharmaceutically acceptable salt:

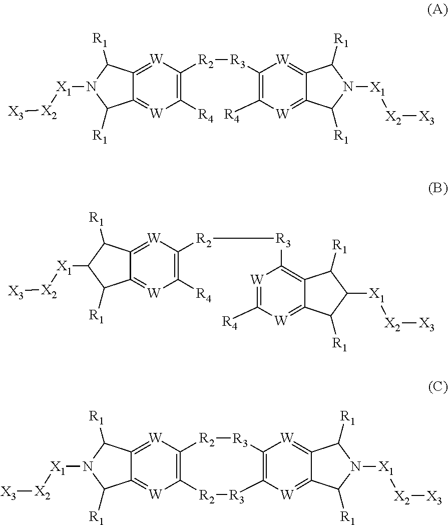

wherein,
each W is independently selected from CR₁ or N;
each R₁ is independently selected from H, deuterium, halogen, OR⁶, N(R⁶)₂, COOR⁶, or C(O)N(R⁶)₂, CN or C₁-C₆ alkyl; wherein the C₁-C₆ alkyl is optionally substituted with one or more deuterium, halogen, OR⁶, N(R⁶)₂, COOR⁶, or C(O)N(R⁶)₂;
R₂ and R₃ are independently selected from the group consisting of O—(C₁-C₄ alkylene or haloalkylene), C₁-C₅ alkylene or haloalkylene, N(R⁶)—(C₁-C₄ alkylene or haloalkylene), -Tₐ-C₁-C₆ alkyl-T_{b}-, -Tₐ- N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}-, - Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃-C₁₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ- (C₃-C₁₂cycloalkyl)-T_{b}-, -Tₐ- (C₆-C₁₂ aryl)-T_{b}-, -Tₐ-(3- to 12-membered heterocycloalkyl)-T_{b}-, or -Tₐ-(5to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O— (C₁-C₆ alkyl)-O—; wherein the C₁-C₆ alkyl is optionally substituted with one or more halogen; and
each Rs independently is H, deuterium or C₁-C₆ alkyl optionally substituted with one or more halogen;
each R⁴ is independently selected from the group consisting of H, deuterium, halogen, CN, OR⁶, N(R⁶)₂, COOR⁶, C(O)N(R⁶)₂, SO₂R⁶, C₁-C₆ alkyl, C₁-C₆ haloalkyl, C₁-C₆ alkyl substituted by OR⁶, C₂-C₆ alkenyl, C₂-C₆ haloalkenyl, C₂-C₆ alkenyl substituted by OR⁶, C₂-C₆ alkynyl, C₂-C₆ haloalkynyl, C₂-C₆ alkynyl substituted by OR⁶, C₃-C₆ cycloalkyl, and a 3- to 6-membered heterocyclic ring including 1 to 2 ring members selected from the group consisting of O, S, and N(R⁶);
each R⁶ is independently selected from the group consisting of —H, deuterium, halogen, —NH₂, —CN, —OH, —N₃, —NO₂, carboxyl, C₁₋₆alkyl, C₁₋₆alkoxy, C₂₋₆alkenyl, C₂₋₆alkynyl, —C₆₋₁₀ aryl, —C₅₋₁₀heteroaryl, C₃₋₁₀heterocyclic ring or carbocyclic ring; and each of which is independently optionally substituted with deuterium, halogen, —NH₂, —CN, —OH, —NO₂, carbonyl, ═O, oxo, carboxyl, C₁₋₆alkoxy, or C₁₋₆alkyl; and each of the heteroaryl and heterocyclic ring contains at least one heteroatoms selected from N, O or S;
each X₁ is independently selected from the group consisting of C═O, —CH₂—, —CHF—, and —CF₂—;
each X₂ is independently selected from (C(R⁸)₂)₍₁₋₃₎, NR⁸(C(R⁸)₂)₍₁₋₃₎, —NH(C(R⁸)₂)₍₁₋₃₎, —N(C₁₋₆alkyl)(C(R⁸)₂)₍₁₋₃₎ or —N(haloC₁₋₆alkyl) (C(R⁸)₂)₍₁₋₃₎; wherein each R⁸ is independently selected from the group consisting of H, deuterium, halogen, C₁-C₆ alkyl, CN, OR⁶, N(R⁶)₂, C₁-C₆ haloalkyl, C₃-C₆ cycloalkyl, C₁-C₆ alkyl substituted by OR⁶, and C₁-C₆ alkyl substituted by N(R⁶)₂; optionally 2 R⁸ on different carbon atoms may be taken together, along with the atoms to which they are attached, to form a 3- to 6-membered fused ring; and optionally 2 R⁸ on a single carbon atom may be taken together, along with the atom to which they are attached, to form a 3- to 6-membered spirocycle;
each X₃ is independently selected from the group consisting of COOR⁶, C(O)SR⁶, C(S)OR⁶, SO₂R⁶, C(O)N(R⁹)₂,

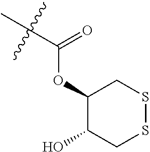 [SMILES: *C(=O)O[C@H]1CSSC[C@@H]1O]

and CN; and each R⁹ is independently selected from H, deuterium, COOR⁶, SO₂R⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆alkyl), S(C₆-C₁₀ aryl), S(═O)₂R⁶, S(═O)₂O R⁶, P(═O)(R⁶)₂, C₁-C₆alkyl, C₂-C₆ alkenyl, C₂-C₆ alkynyl, C₃-C₅ cycloalkyl, C₆-C₁₀aryl, 3-8 membered heterocycloalkyl, or 3-10 membered heteroaryl.

In some embodiments of Formula (A),

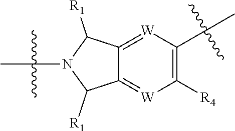

is independently selected from

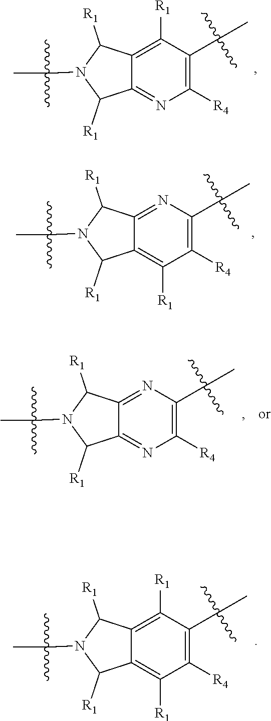

In some embodiments of Formula (A),

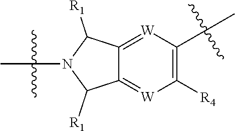

is independently selected from

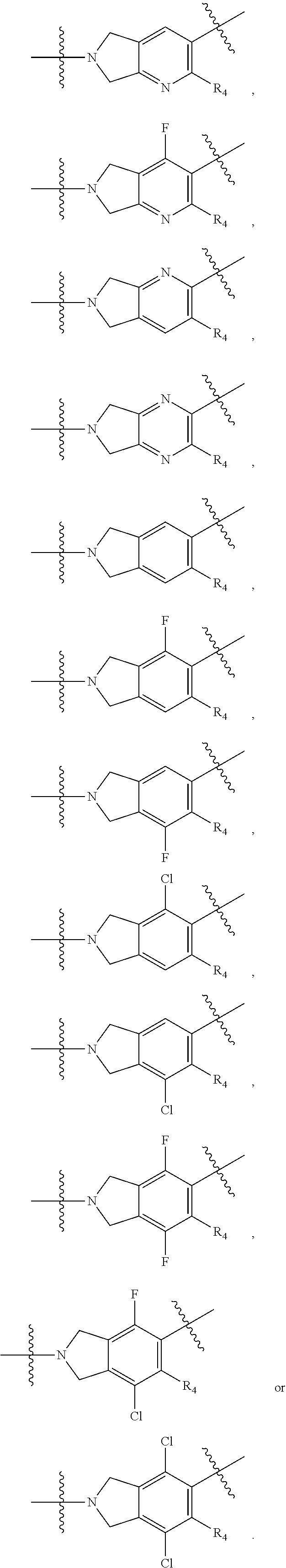

In some embodiments of Formula (A), the compound is of Formula (A-1):

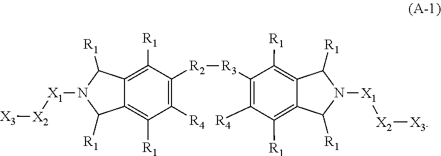

In some embodiments of Formula (B),

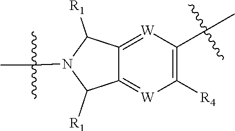

is independently selected from

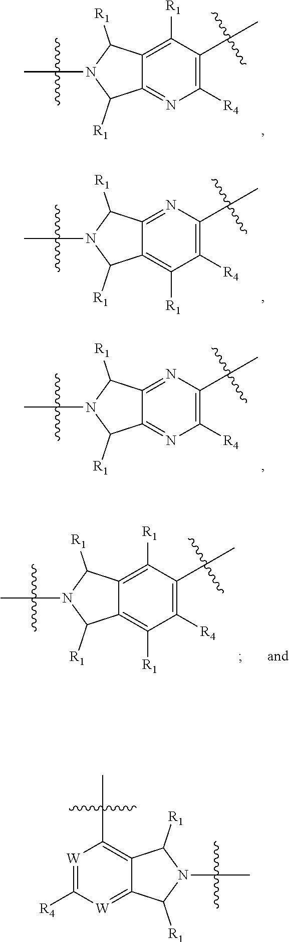

is independently selected from

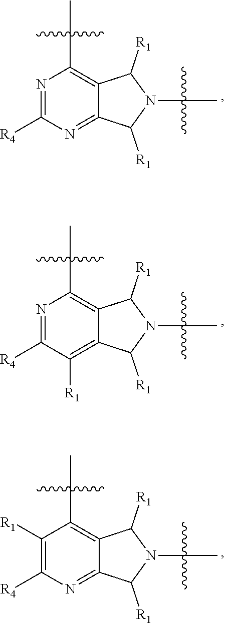

In some embodiments of Formula (B),

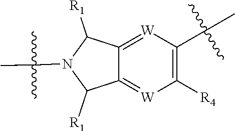

is independently selected from

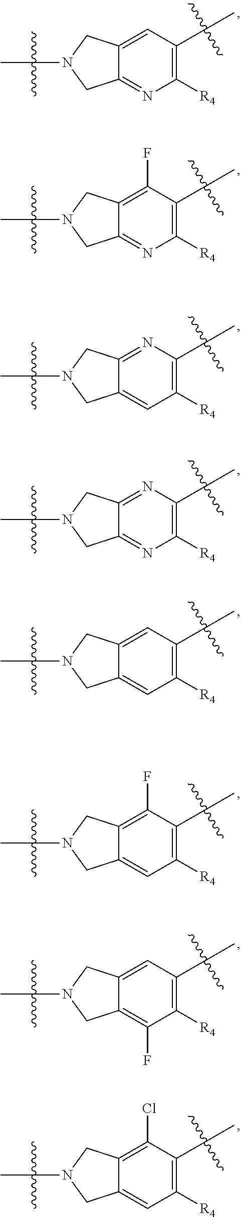

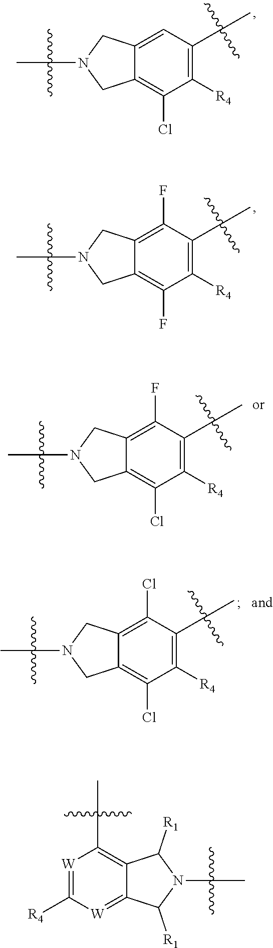

is independently selected from

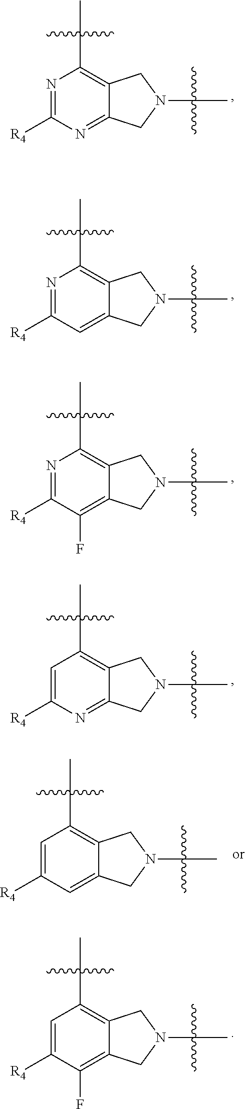

In some embodiments of Formula (C),

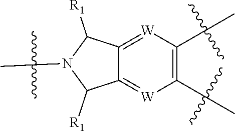

is independently selected from

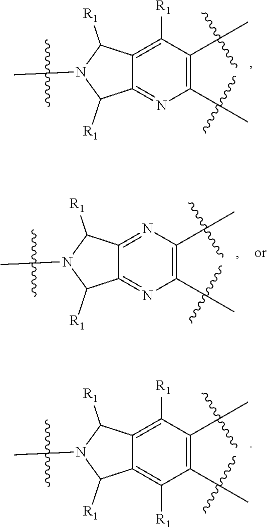

In some embodiments of Formula (C),

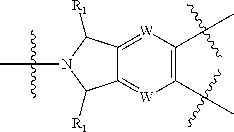

is independently selected from

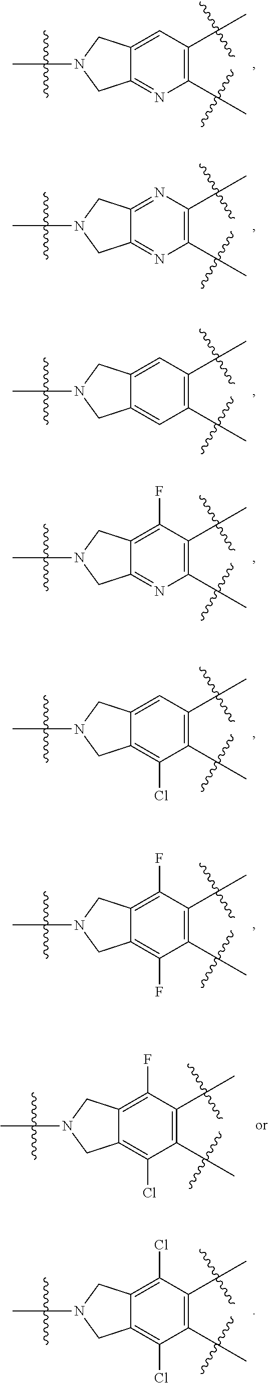

In some embodiments of Formula (A), (A-1), (B) or (C), each W is independently CR₁.

In some embodiments of Formula (A), (A-1), (B) or (C), each W is independently is CH or CF.

In some embodiments of Formula (A), (A-1), (B) or (C), each W is independently N.

In some embodiments of Formula (A), (A-1), (B) or (C), each R₁ is independently selected from H, deuterium, halogen, OR⁶, N(R⁶)₂, CN or C₁-C₆ alkyl; wherein the C₁-C₆ alkyl is optionally substituted with one or more deuterium, halogen, OR⁶, N(R⁶)₂, COOR⁶, or C(O)N(R⁶)₂.

In some embodiments of Formula (A), (A-1), (B) or (C), each R₁ is independently selected from the group consisting of H, deuterium, halogen, C₁-C₃ alkyl, CN and C₁-C₃ haloalkyl.

In some embodiments of Formula (A), (A-1), (B) or (C), wherein each R₁ is independently selected from the group consisting of H, deuterium, halogen, CN and C₁-C₃ alkyl.

In some embodiments of Formula (A), (A-1), (B) or (C), each R₁ is independently selected from the group consisting of H, deuterium, F, Cl, Br, CN and methyl.

In some embodiments, each R₁ independently is hydrogen or halogen.

In some embodiments, each R₁ independently is hydrogen or F.

In some embodiments, each R₁ independently is hydrogen or CN.

In some embodiments, each R₁ independently is deuterium.

In some embodiments of Formula (A), (A-1), (B) or (C), R₂ and R₃ are independently selected from -Tₐ C₁-C₆ alkyl-T_{b}-, -Tₐ- N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}-, -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃-C₁₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ S(═O)₂-T_{b}-, -Tₐ-S(═O)-T_{b}-, -Tₐ-P(═O)(—ORs)-T_{b}-, -Tₐ- (C₃-C₁₂cycloalkyl)-T_{b}-, -Tₐ- (C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs.

In some embodiments of Formula (A), (A-1), (B) or (C), R₂ and R₃ are independently selected from -Tₐ C₁-C₆ alkyl-T_{b}-, -Tₐ- N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}-, -Tₐ C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃-C₁₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ S(═O)₂-T_{b}-, -Tₐ-S(═O)-T_{b}-, -Tₐ-P(═O)(—ORs)-T_{b}-, -Tₐ- (C₃-C₁₂cycloalkyl)-T_{b}-, -Tₐ- (C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —ORs, —N(Rs)₂, or —C(═O)ORs. Wherein in -Tₐ- (C₃-C₁₂ cycloalkyl)-T_{b}- or -Tₐ-(3- to 12-membered heterocycloalkyl)-T_{b}-, the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O— (C₁-C₆ alkyl)-O—; wherein the C₁-C₆ alkyl is optionally substituted with one or more halogen; and
each Rs independently is H, deuterium or C₁-C₆ alkyl optionally substituted with one or more halogen.

In some embodiments of Formula (A), (A-1), (B) or (C), R₂ and R₃ are independently selected from -Tₐ-C₁-C₆ alkyl-T_{b}-, -Tₐ- N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}-, -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═S)- T_{b}-, -Tₐ-S(═O)₂-T_{b}-, -Tₐ-S(═O)-T_{b}-, -Tₐ-P(═O)(—ORs)-T_{b}-, -Tₐ- (C₃-C₁₂cycloalkyl)-T_{b}-, -Tₐ- (C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs.

In some embodiments of Formula (A), (A-1), (B) or (C), R₂ and R₃ are independently selected from -Tₐ-C₁-C₆ alkyl-T_{b}-, -Tₐ- N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}-, -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═S)- T_{b}-, -Tₐ-S(═O)₂-T_{b}-, -Tₐ-S(═O)-T_{b}-, -Tₐ-P(═O)(—ORs)-T_{b}-, -Tₐ- (C₃-C₁₋₂cycloalkyl)-T_{b}-, -Tₐ- (C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —ORs, —N(Rs)₂, or —C(═O)ORs; and wherein the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O— (C₁-C₆ alkyl)-O—; wherein the C₁-C₆ alkyl is optionally substituted with one or more halogen; and each Rs independently is H, deuterium or C₁-C₆ alkyl optionally substituted with one or more halogen.

In some embodiments of Formula (A), (A-1), (B) or (C), R₂ and R₃ are independently selected from 0-(C₁-C₄ alkylene or haloalkylene)-C₂-C₆alkenyl, C₁-C₅ alkylene or haloalkylene, (C₁-C₄ alkylene or haloalkylene)-N(R⁶), and N(R⁶)—(C₁-C₄ alkylene or haloalkylene)- C₂-C₆alkenyl, —C₀₋₆alkyl-NH—C₀₋₆alkyl-, —Co₆alkyl-N(C₁₋₆alkyl)-C₀₋₆alkyl-, —C₀₋₆alkyl-O—C₀₋₆alkyl-, —C₀₋₆alkyl-PEGₙ-O—C₀₋₆alkyl, —C₀₋₆alkyl-S—S—C₀₋₆alkyl, —C₀₋₆alkyl-S—S—S—C₀₋₆alkyl, —C₀₋₆ alkyl-C₂-C₆alkenyl-C₀₋₆ alkyl-, —C₀₋₆ alkyl-C₂-C₆alkynyl- C₀₋₆ alkyl-, —C₀₋₆ alkyl-C(═O)—C₀₋₆ alkyl-, —C₀₋₆ alkyl-C(═CH₂)—C₀₋₆alkyl-, —C₀₋₆ alkyl-C(═O)—C(═O)—C₀₋₆alkyl-, —C₀₋₆ alkyl-C(=S)—C₀₋₆alkyl-, —C₀₋₆ alkyl-S(═O)₂—C₀₋₆alkyl-, —C₀₋₆alkyl-S(═O)—C₀₋₆alkyl-, —C₀₋₆ alkyl-P(═O)(—OH)—C₀₋₆alkyl-, —C₀₋₆ alkyl-C₃-C₁₂ cycloalkyl-C₀₋₆alkyl-, —C₀₋₆ alkyl-C₆-C₁₂ aryl-C₀₋₆alkyl-, —C₀₋₆ alkyl-(3- to 12-membered heterocyclyl)-C₀₋₆ alkyl-, —C₀₋₆ alkyl-(5- to 12-membered heteroaryl)-C₀₋₆ alkyl-, —C₀₋₆alkyl-O-(5- to 12-membered heteroaryl)-O—C₀₋₆alkyl-, —C₀₋₆alkyl-O—C(═O)—NH—C₀₋₆alkyl-, —C₀₋₆alkyl-O—C(═O)—C₀₋₆alkyl-, —C₀₋₆alkyl-NH—C(═O)—C₀₋₆alkyl-, —OC(═O)—O—, —NH—C(═O)—NH—, or —NH—C(═S)—NH—; wherein the C₂-C₆ alkenyl, C₂-C₆alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs.

In some embodiments of Formula (A), (A-i), (B) or (C), R₂ and R₃ are independently selected from TₐC₂-C₆ alkenyl-T_{b}-, -Tₐ-C(═O)-T_{b}-, -Tₐ-PEGₙ-O-T_{b}, -Tₐ-S—S-T_{b}, -Tₐ-S—S—S-T_{b}, -Tₐ C(=CH₂)-T_{b}-, or -Tₐ- (C₆-C₁₂ aryl)-T_{b}-, wherein the C₂-C₆alkenyl or C₆-C₁₂ aryl is optionally substituted with one or more halo, —ORs, —N(Rs)₂, or —C(═O)ORs;
PEGₙ is (—OCH₂CH₂—)ₙ, n=1-8;
Tₐ and T_{b} each independently are —N(Rs)-, —O—, —(C₁-C₆ alkyl)-O—, or —O—(C₁-C₆ alkyl)-O—; wherein the C₁-C₆alkyl it optionally substituted with one or more halogen; each Rs independently is H, deuterium or C₁-C₆ alkyl optionally substituted with one or more halogen.

In some embodiments of Formula (A), (A-i), (B) or (C), wherein R₂—R₃ is selected from

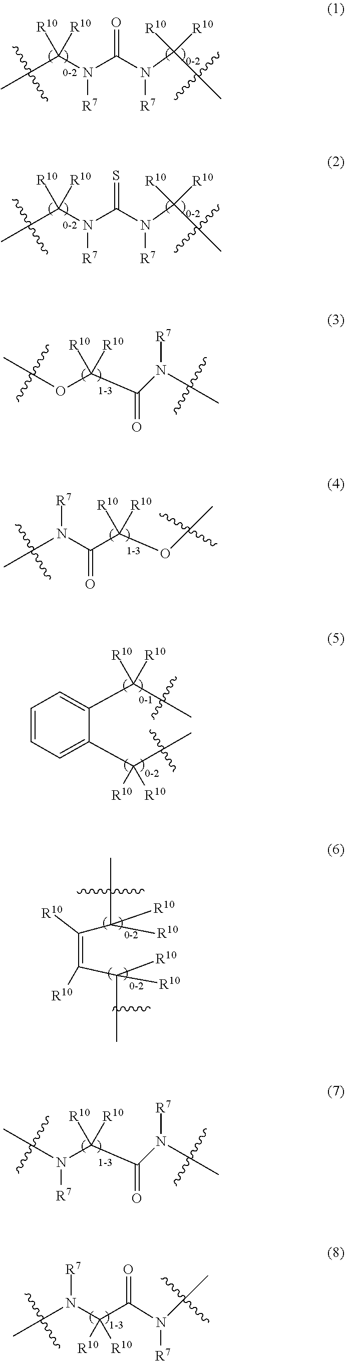

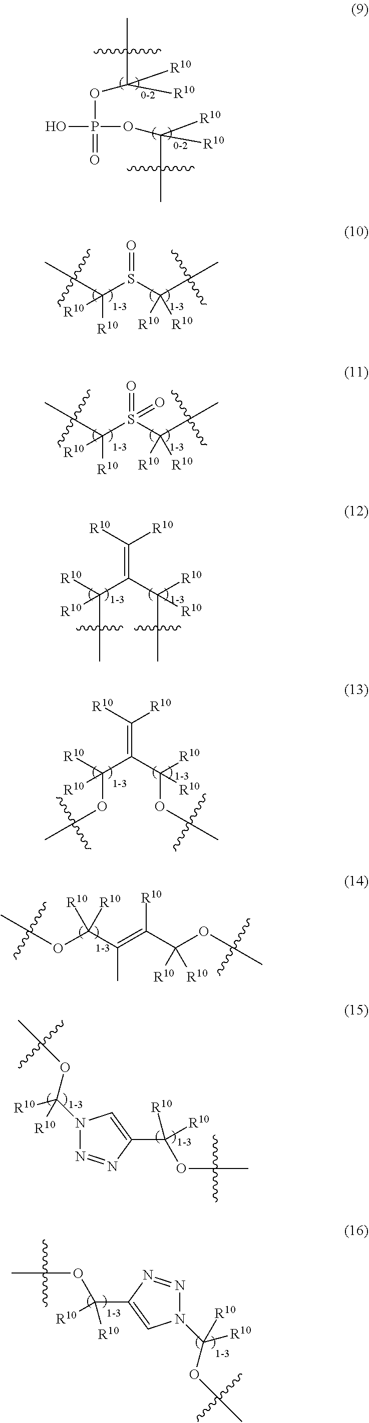

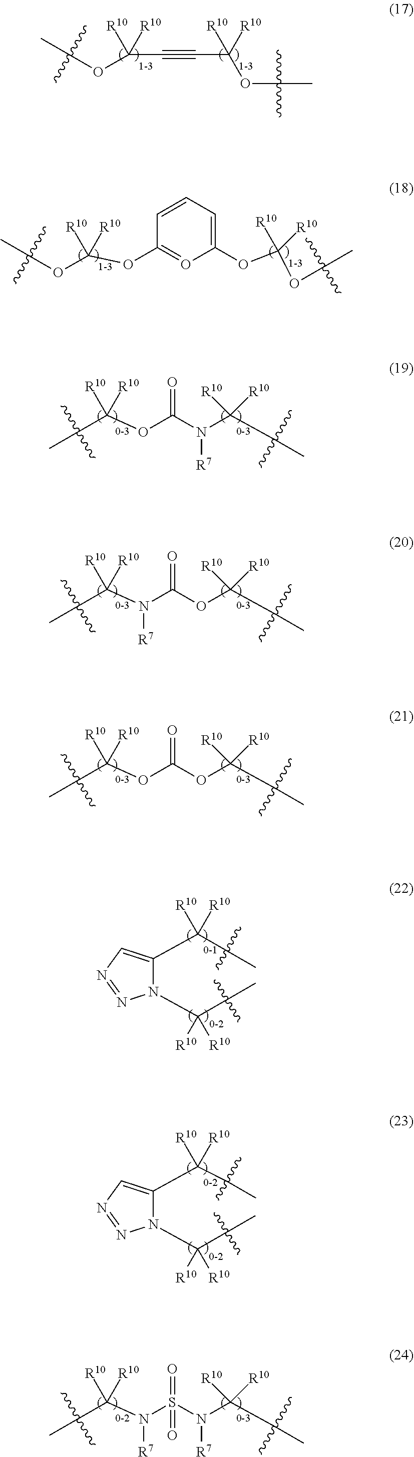

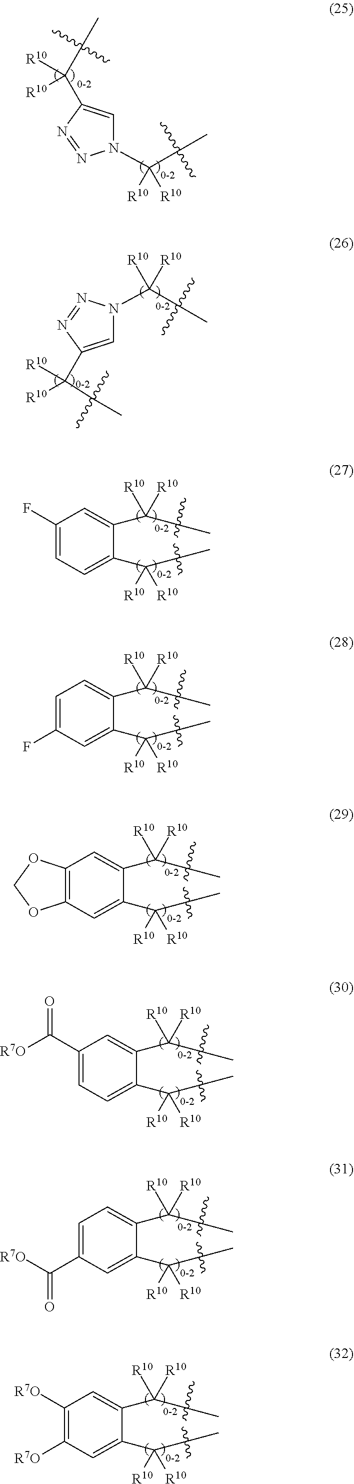

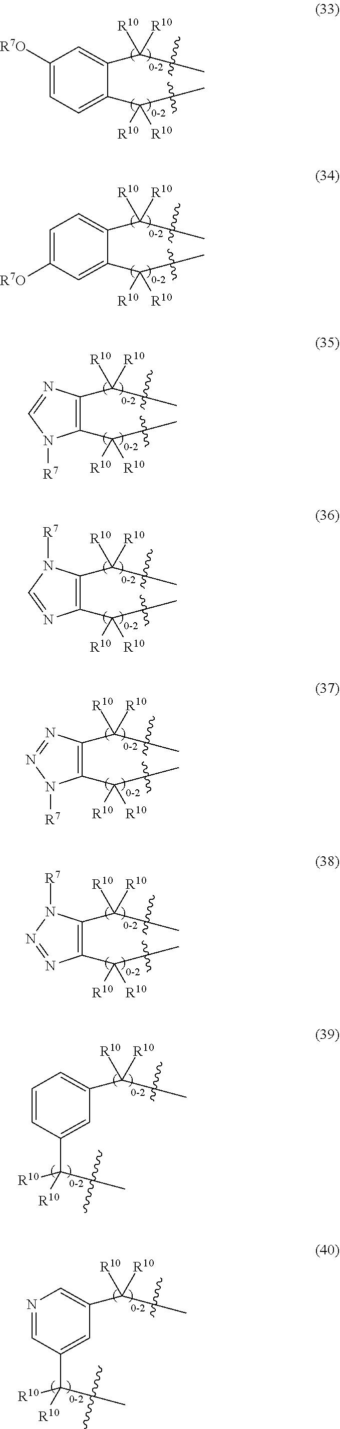

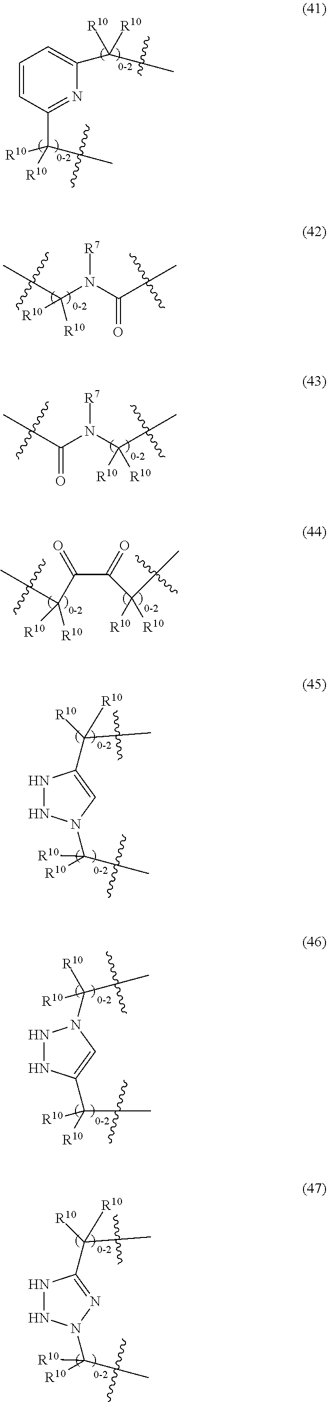

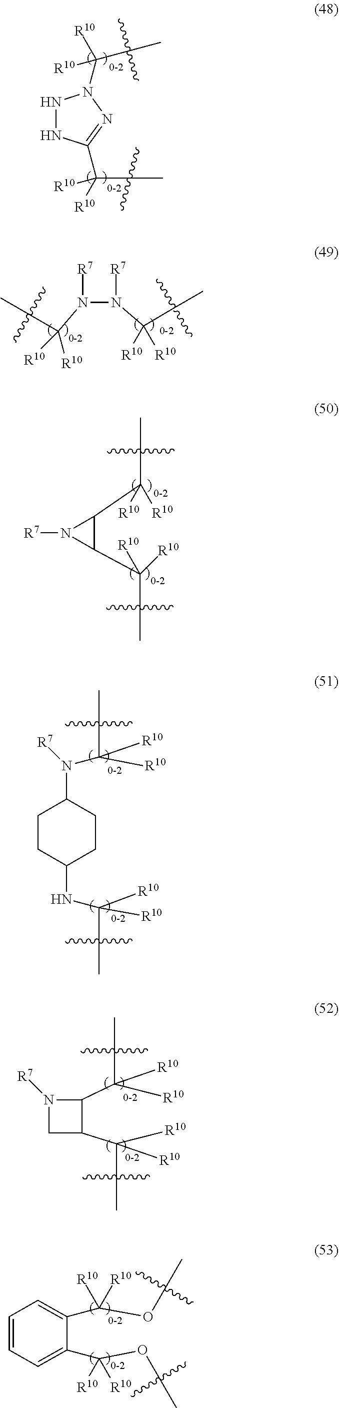

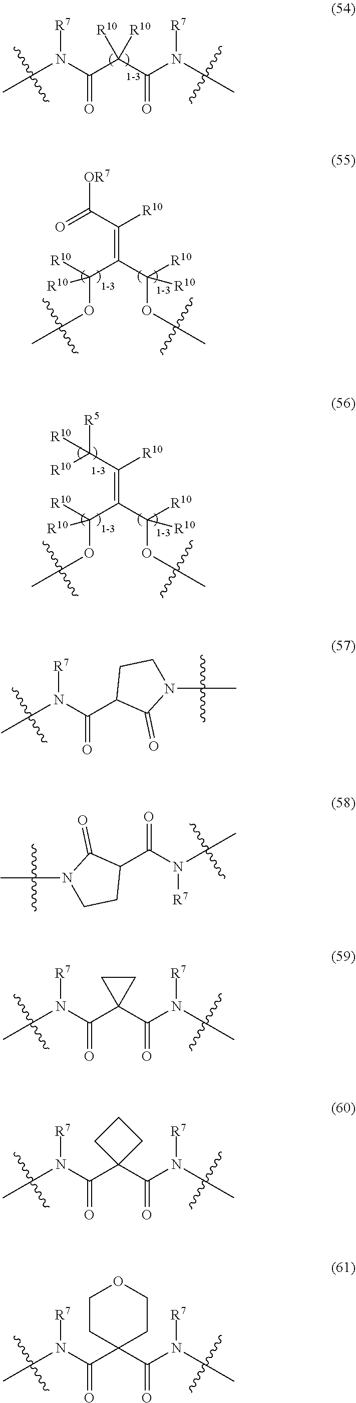

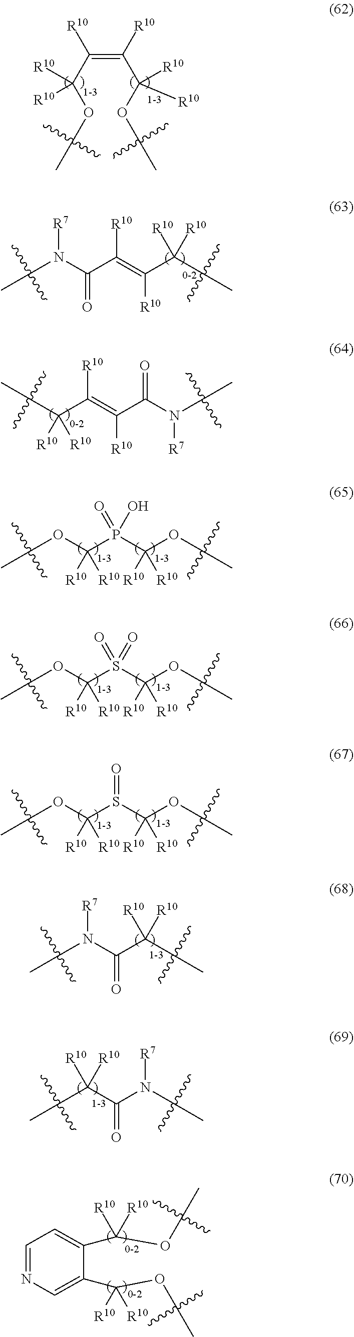

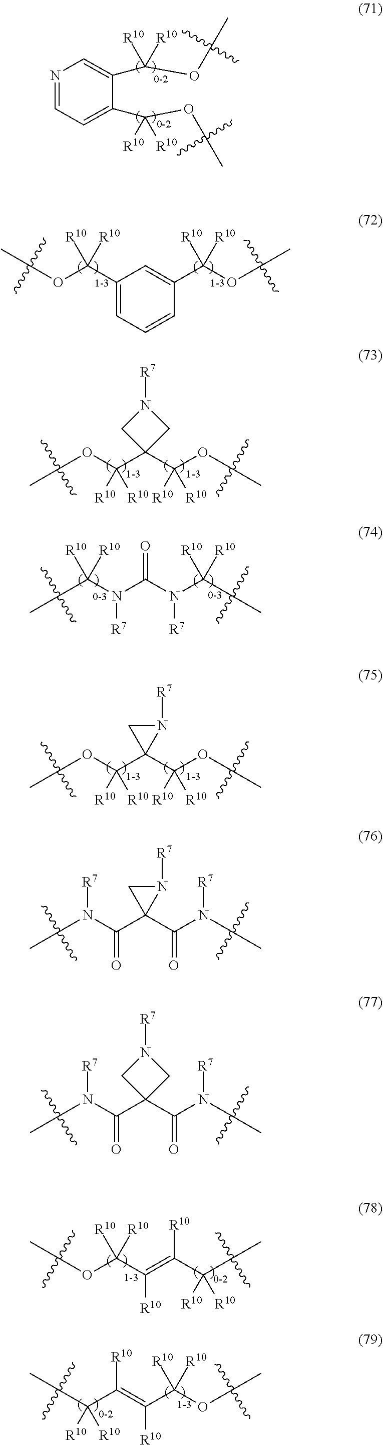

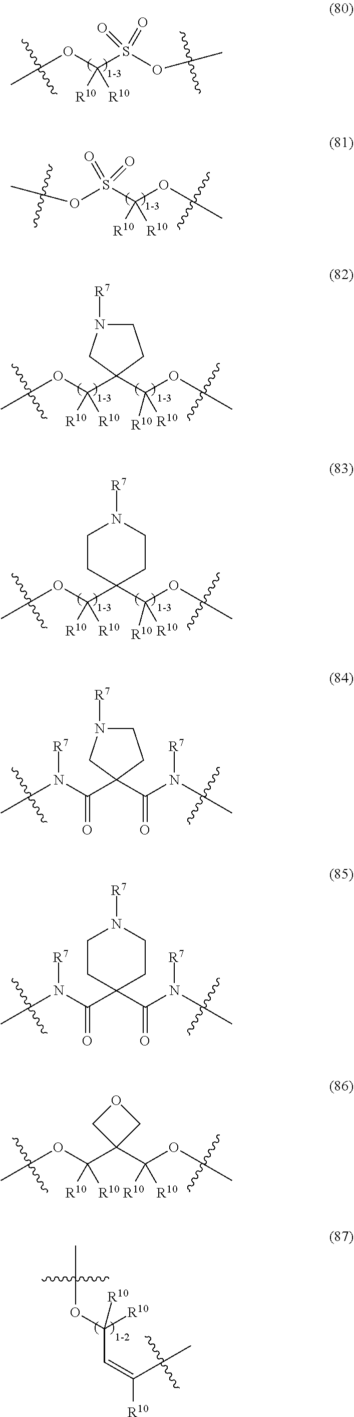

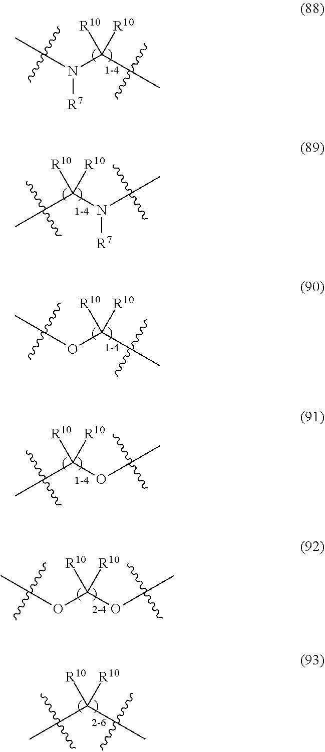

In some embodiments, each R⁷ is independently hydrogen, deuterium, or methyl.

In some embodiments, each R¹⁰ is independently hydrogen, deuterium, methyl or fluorine.

In some embodiments, one R¹⁰ is hydrogen, and the other R¹⁰ is methyl or fluorine.

In some embodiments, one R¹⁰ is deuterium, and the other R¹⁰ is methyl or fluorine.

In some embodiments of Formula (A), (A-1), (B) or (C), wherein R₂—R₃ is selected from the group consisting of —(CH₂)₂₋₈—, —O(CH₂)₁₋₇—, —O(CH₂)₁₋₆O—, —OCH₂CH(CH₃)CH₂O—, —OCH(CH₃)CH₂CH(CH₃)O—,

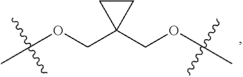 [SMILES: *OCC1(CO*)CC1]

—NH(CH₂)₁—, —(CH₂)₁₋₆NH(CH₂)₁₋₆—, —(CH₂)₁₋₆N(CH₃)(CH₂)₁₋₆—, —NH(CH₂)₁₋₆O—, —NH—CO—NH—, —N(CH₃)CO—NH—,

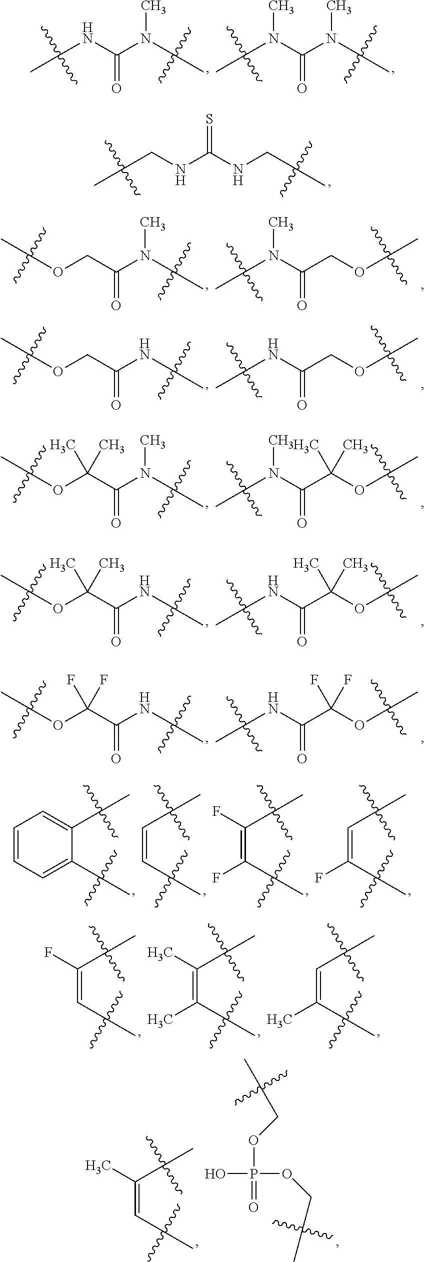

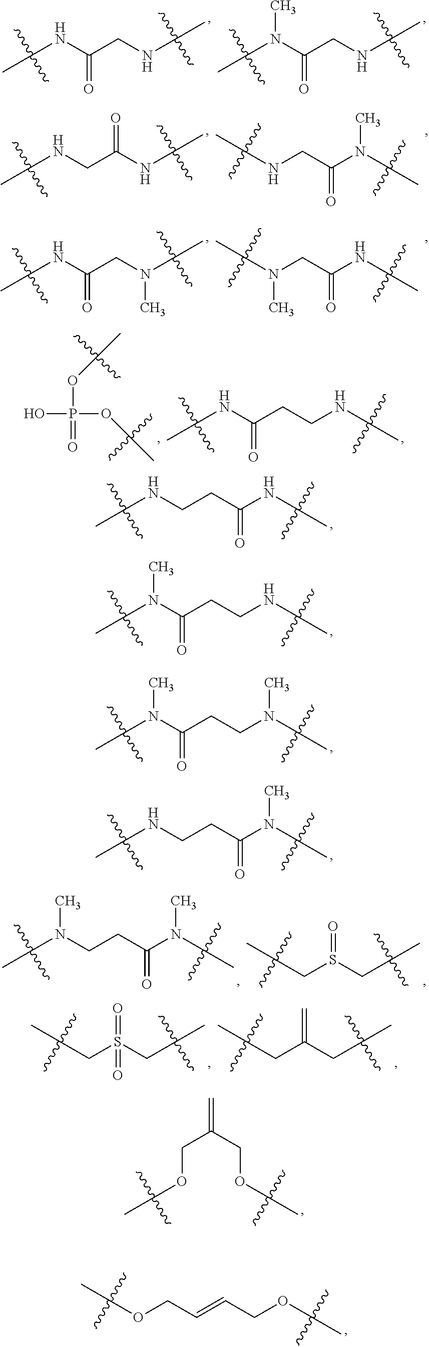

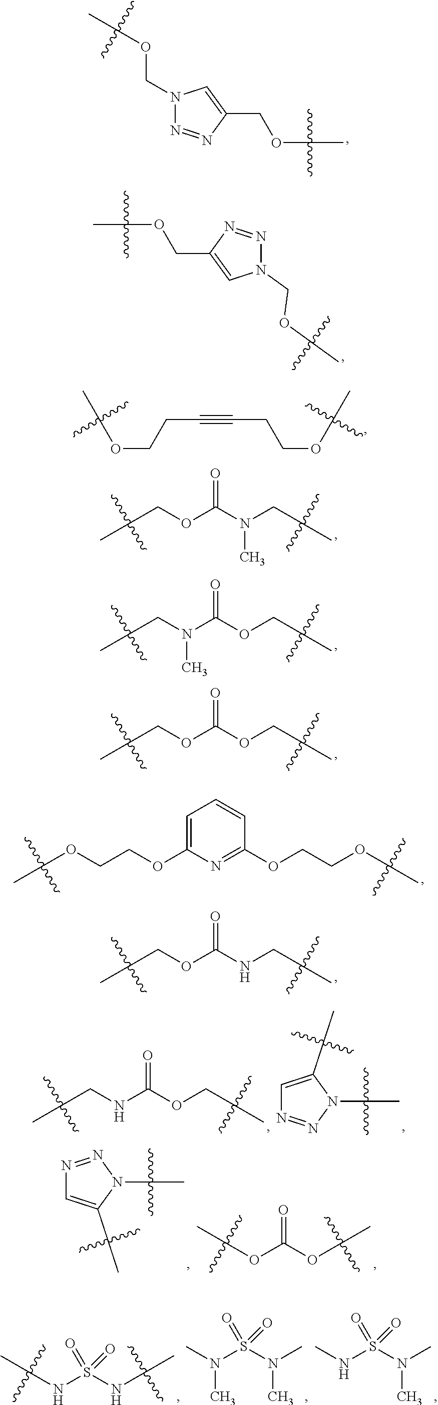

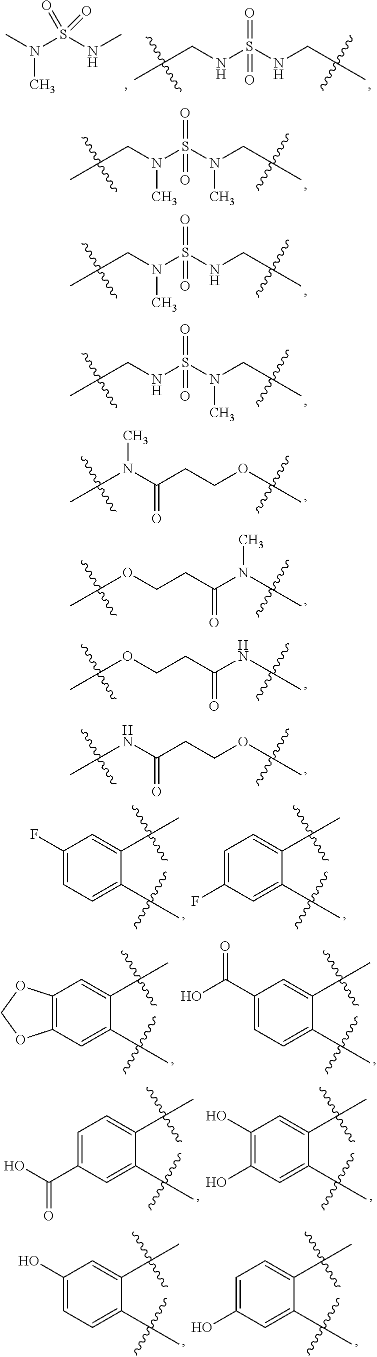

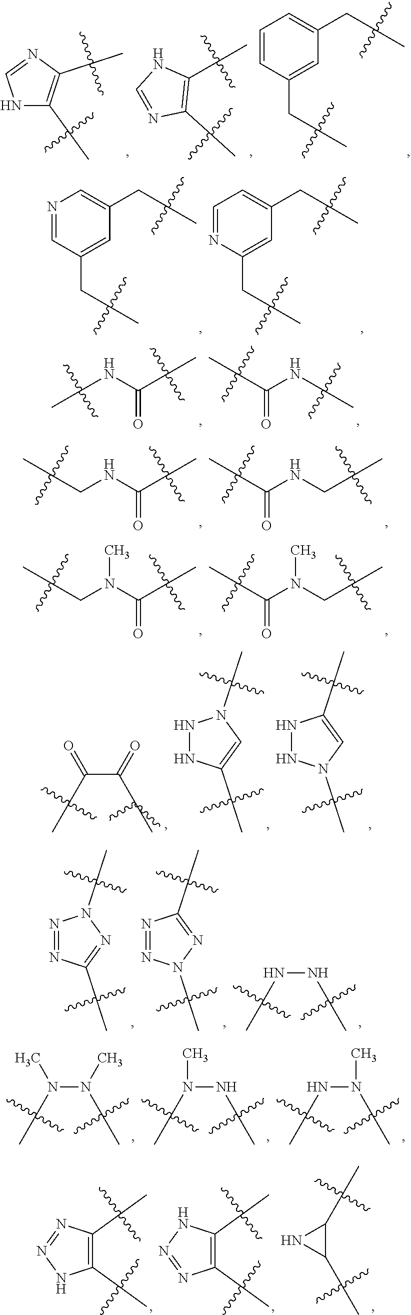

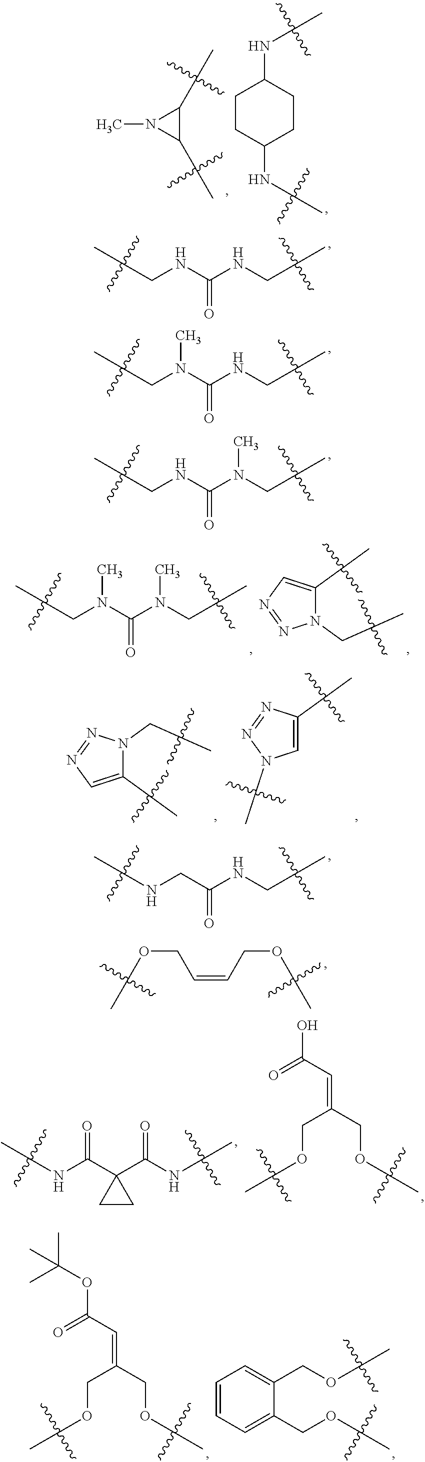

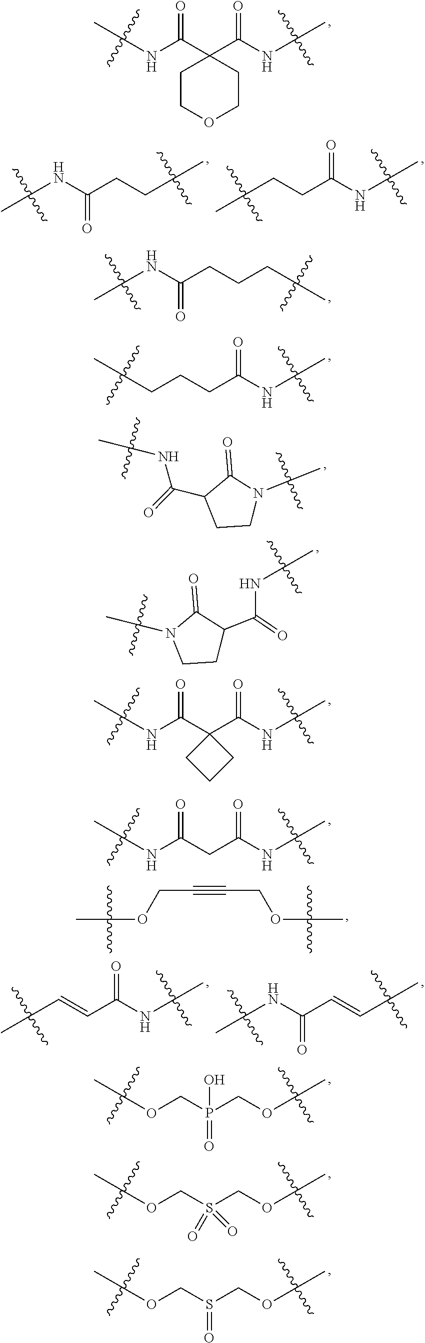

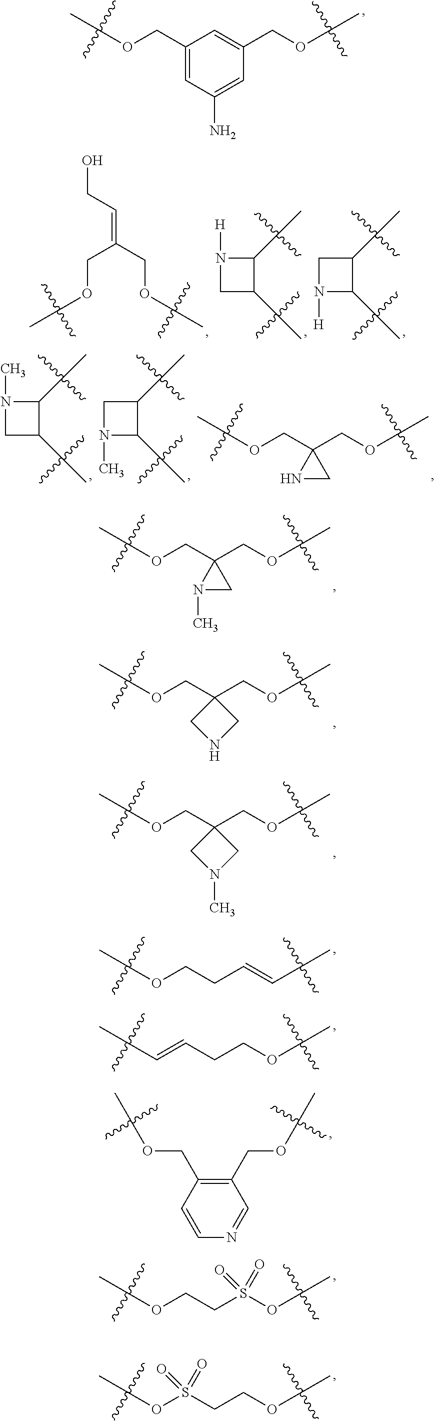

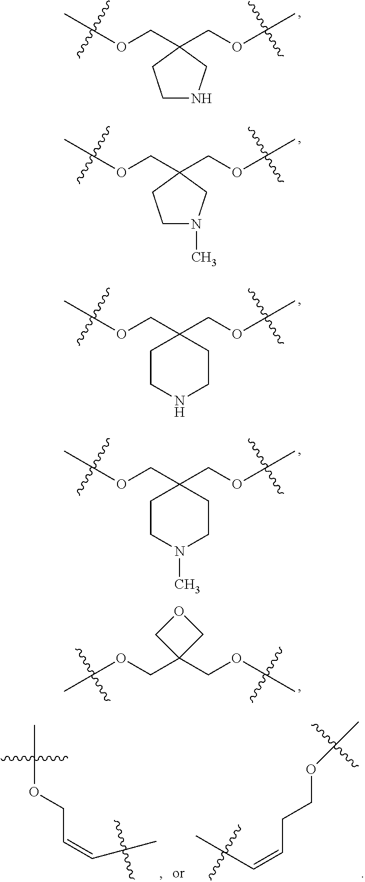

In some embodiments of Formula (A), (A-1), (B) or (C), wherein R²—R³ is selected from the group consisting of —(CH₂)₂—, (CH₂)₃—, (CH₂)₄—, —(CH₂)₅—, O(CH₂)—, —O(CH₂, O(CH₂)₃O—, —O(CH₂)₄O—, —OCH₂CH(CH₃)CH₂O—, —OCH(CH₃)CH₂CH(CH₃)O—,

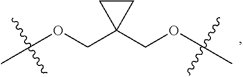 [SMILES: *OCC1(CO*)CC1]

O(CH₂)₄O—, —O(CH₂)₅O—, —NH(CH₂)₂—, —NH(CH₂)₃—, —NH(CH₂)₄—, —(CH₂)₂NH—, —(CH₂)₃NH—, —(CH₂)₄NH— —CH₂NHCH₂—, —CH₂N(CH₃)CH₂—, —NH(CH₂)₃O—, —NH—CO—NH—,

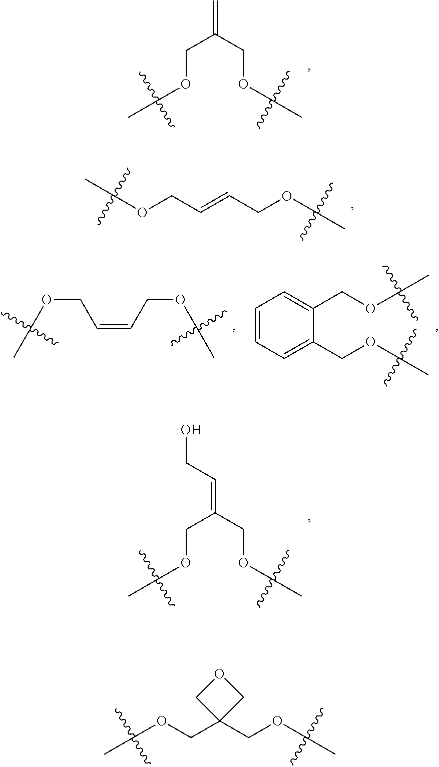

### or —N(CH₃)CONH—.

In some embodiments of Formula (A), (A-1) or (B), each R⁴ is independently selected from the group consisting of H, deuterium, halogen, CN, ORH, N®₂, COOR⁶, C(O)N(R⁶)₂, SO₂R⁶, C₁-C₆ alkyl, C₁—C haloalkyl, C₁-C₆ alkyl substituted by OR⁶, C₂-C₆ alkenyl, C₂-C₆ haloalkenyl, C₂-C₆ alkenyl substituted by OR⁶, C₂-C₆ alkynyl, C₂-C₆ haloalkynyl, C₂-C₆ alkynyl substituted by OR⁶, C₃-C₆ cycloalkyl, and a 3- to 6-membered heterocyclic ring including 1 to 2 ring members selected from the group consisting of O, S, and N(R⁶).

In some embodiments of Formula (A), (A-1) or (B), each R⁴ is independently selected from the group consisting of H, deuterium, F, Cl, Br, I, OH, C₁-C₃ alkyl, C₁-C₃ haloalkyl, OC, —C₃ alkyl, OC₁-C₃ haloalkyl, C₂-C₃alkenyl, C₂-C₃alkynyl, and N(R⁶)₂.

In some embodiments of Formula (A), (A-1) or (B), each R⁴ independently is selected from the group consisting of H, deuterium, Br, C₁, OH, CH₃, CH₂CH₃, CH═CH₂, C≡CH, OCH₃, OCFH₂, OCF₂H, OCF₃, and N(R⁶)₂.

In some embodiments of Formula (A), (A-1) or (B), each R⁴ independently is selected from the group consisting of H, deuterium, Br, OH, CH₃, CH₂CH₃, CH═CH₂, C≡CH, OCH₃, NH₂ and NHCH₃.

In some embodiments of Formula (A), (A-1), (B) or (C), each R⁶ is independently selected from the group consisting of —H, deuterium, —F, —Cl, —Br, —I, —NH₂, —CN, —OH, —N₃, —NO₂, carboxyl, C₁-C₃alkyl, C₁-C₃alkoxy, C₂-C₄alkenyl, C₂-C₄alkynyl, 6-membered aryl, 7-membered aryl, 8-membered aryl, 5-membered heteroaryl, 6-membered heteroaryl, 7-membered heteroaryl, 8-membered heteroaryl, 5-membered heterocyclic ring, 6-membered heterocyclic ring, 7-membered heterocyclic ring, 8-membered heterocyclic ring, 5-membered carbocyclic ring, 6-membered carbocyclic ring, 7-membered carbocyclic ring, or 8-membered carbocyclic ring; and each of which is independently optionally substituted with deuterium, —F, —Cl, —Br, —I, —NH₂, —CN, —OH, —NO₂, carbonyl, ═O, oxo, carboxyl, C₁-C₃alkoxy, or C₁-C₃alkyl; and each of the heteroaryl and heterocyclic ring contains 1 or 2 heteroatoms selected from N, O or S.

In some embodiments of Formula (A), (A-1), (B) or (C), each R⁶ is independently selected from the group consisting of —H, deuterium, —F, —Cl, —Br, —I, —NH₂, —CN, —OH, —N₃, —NO₂, carboxyl, methyl, ethyl, propyl, isopropyl, methoxy, ethoxy, propoxy, isopropoxy, ethylene, 6-membered aryl, 5-membered heteroaryl, 6-membered heteroaryl, 5-membered heterocyclic ring, 6-membered heterocyclic ring, 5-membered carbocyclic ring, or 6-membered carbocyclic ring; and each of which is independently optionally substituted with deuterium, —F, —Cl, —Br, —I, —NH₂, —CN, —OH, —NO₂, carbonyl, ═O, oxo, carboxyl, methyl, ethyl, propyl, isopropyl, methoxy, ethoxy, propoxy or isopropoxy; and each of the heteroaryl and heterocyclic ring contains 1 or 2 heteroatoms selected from N, O or S.

In some embodiments of Formula (A), (A-1), (B) or (C), each R⁶ is independently selected from the group consisting of H, deuterium, —F, —Cl, —Br, —I, —NH₂, —CN, —OH, methyl, ethyl, propyl, isopropyl, methoxy, ethoxy, S propoxy, isopropoxy, CH₂F, —CHF₂, —CF₃ and

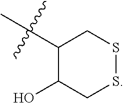 [SMILES: *C1CSSCC1O]

In some embodiments of Formula (A), (A-1), (B) or (C), each X1 is independently selected from the group consisting of C═O, —CH₂—, —CHF—, and —CF₂—.

In some embodiments of Formula (A), (A-1), (B) or (C), each X1 is selected from the group consisting of C═O and —CH₂—.

In some embodiments of Formula (A), (A-1), (B) or (C), each X₁ is C═O.

In some embodiments of Formula (A), (A-1), (B) or (C), each X₂ is independently selected from (C(R⁸)₂)₍₁₋₃₎, wherein each R⁸ is independently selected from the group consisting of H, deuterium, halogen, C₁-C₆ alkyl, CN, OR⁶, N(R⁶)₂, C₁-C₆ haloalkyl, C₃-C₆ cycloalkyl, C₁-C₆ alkyl substituted by OR⁶, and C₁-C₆ alkyl substituted by N(R⁶)₂; optionally 2 R⁸ on different carbon atoms may be taken together, along with the atoms to which they are attached, to form a 3- to 6-membered fused ring; and optionally 2 R⁸ on a single carbon atom may be taken together, along with the atom to which they are attached, to form a 3- to 6-membered spirocycle.

In some embodiments, each X₂ independently is —(C(R⁸)₂)₁₋₃—, wherein each R⁸ independently is hydrogen, deuterium, halogen, C₁-C₆ alkyl, CN, OR⁶, N(R⁶)₂, or C₃-C₆ cycloalkyl; wherein the C₁-C₆ alkyl is optionally substituted with one or more halogen, OR⁶, or N(R⁶)₂.

In some embodiments, each X₂ independently is —(C(R⁸)₂)₁₋₃—, wherein at least two R⁸, together with the one or more atoms to which they are attached, form C₃-C₆ cycloalkyl or 3- to 6-membered heterocycloalkyl.

In some embodiments, each X₂ independently is —C(R⁸)₁₋₃—.

In some embodiments, each X₂ independently is —(CH₂)₁₋₃—.

In some embodiments, each X₂ independently is —C(R⁸)₂—.

In some embodiments, each X₂ independently is —CH₂—.

In some embodiments, each X₂ independently is —C(R⁸)₂C(R⁸)₂—.

In some embodiments, each X₂ independently is —CH₂CH₂—.

In some embodiments, each X₂ independently is —C(R⁸)₂C(R⁸)₂C(R⁸)₂—.

In some embodiments, each X₂ independently is —CH₂CH₂CH₂—.

In some embodiments of Formula (A), (A-1), (B) or (C), each X₂ is CH₂CHR⁸, where R⁸ is selected from the group consisting of H, deuterium, C₁-C₃ alkyl, C₁-C₃ alkyl substituted by OH, C₁-C₃ alkyl substituted by OC₁-C₃ alkyl, and C₃-C₆ cycloalkyl.

In some embodiments of Formula (A), (A-1), (B) or (C), each X₂ is CH₂CHR⁸, wherein R⁸ is selected from the group consisting of H, deuterium, CH₃, CH₂OH, CH₂CH₃, CH₂CH₂CH₃, CH(CH₃)₂, CH₂OCH₃, and cyclopropyl.

In some embodiments of Formula (A), (A-1), (B) or (C), each X₂ is CHR⁸CHR⁸, where each R⁸ is independently selected from the group consisting of H, deuterium, C₁-C₃ alkyl, C₁-C₃ alkyl substituted by OH, C₁-C₃ alkyl substituted by OC₁-C₃ alkyl, and C₃-C₆ cycloalkyl, and optionally the 2 R⁸ on different carbon atoms are taken together, along with the atoms to which they are attached, to form a 3- to 6-membered fused ring.

In some embodiments of Formula (A), (A-1), (B) or (C), each X₂ is CHR⁸CHR⁸, where each R⁸ is independently selected from the group consisting of H, deuterium and C₁-C₃ alkyl, and optionally the 2 R⁸ on different carbon atoms are taken together, along with the atoms to which they are attached, to form a 3- to 6-membered fused ring.

In some embodiments of Formula (A), (A-1), (B) or (C), each X₂ is CH₂C(R⁸)₂, where each R⁸ is independently selected from the group consisting of H, deuterium, C₁-C₃ alkyl, C₁-C₃ alkyl substituted by OH, C₁-C₃ alkyl substituted by OC₁-C₃ alkyl, and C₃-C₆ cycloalkyl, and optionally the 2 R⁸ on a single carbon atom are taken together, along with the atoms to which they are attached, to form a 3- to 6-membered spirocycle.

In some embodiments of Formula (A), (A-1), (B) or (C), each X₂ is CH₂C(R⁸)₂, where each R⁸ is independently selected from the group consisting of H, deuterium and C₁-C₃ alkyl, and optionally the 2 R⁸ on a single carbon atom are taken together, along with the atoms to which they are attached, to form a 3- to 6-membered spirocycle.

In some embodiments of Formula (A), (A-1), (B) or (C), each X₃ is independently selected from the group consisting of COOR⁶, C(O)SR⁶, C(S)OR⁶, SO₂R⁶, C(O)N(R⁹)₂,

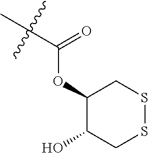 [SMILES: *C(=O)O[C@H]1CSSC[C@@H]1O]

### and CN.

In some embodiments of Formula (A), (A-1), (B) or (C), each X₃ is independently selected from the group consisting of COOR⁶, SO₂R⁶, C(O)N(R⁹)₂,

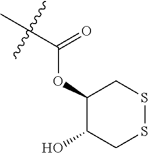 [SMILES: *C(=O)O[C@H]1CSSC[C@@H]1O]

### and CN.

In some embodiments of Formula (A), (A-1), (B) or (C), each X₃ is independently selected from the group consisting of COOR⁶, C(O)N(R⁹)₂,

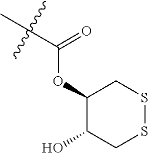 [SMILES: *C(=O)O[C@H]1CSSC[C@@H]1O]

### and CN.

In some embodiments of Formula (A), (A-1), (B) or (C), each X₃ is independently selected from the group consisting of COOH, COOCH₃, CONH₂,

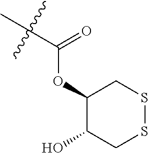 [SMILES: *C(=O)O[C@H]1CSSC[C@@H]1O]

### and CN.

In some embodiments of Formula (A), (A-1), (B) or (C), each R⁹ is independently selected from the group consisting of H, deuterium, COOR⁶, and SO₂R⁶.

In some embodiments of Formula (A), (A-1), (B) or (C), each R⁹ is independently H or deuterium, preferably H.

In some embodiments of Formula (A), (B), or (C), the compound is of Formula (Aa), (Ba), or (Ca):

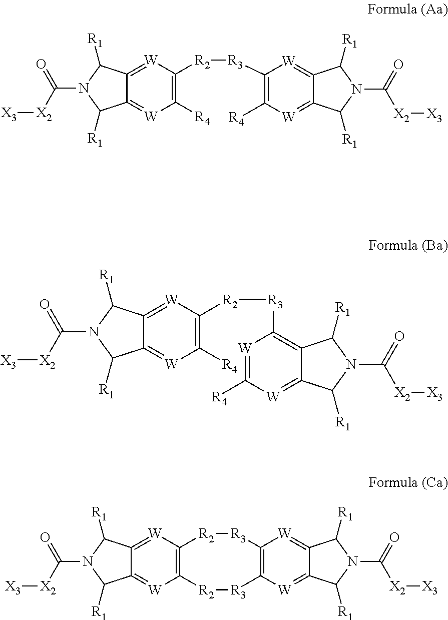

or a pharmaceutically acceptable salt thereof.

In some embodiments of Formula (A) or (B), the compound is of Formula (Ab), or (Bb):

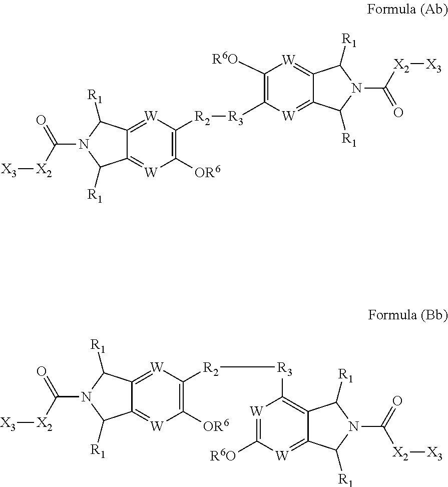

or a pharmaceutically acceptable salt thereof.

In some embodiments of Formula (A), (B), or (C), the compound is of Formula (Ac), (Bc), or (Cc):

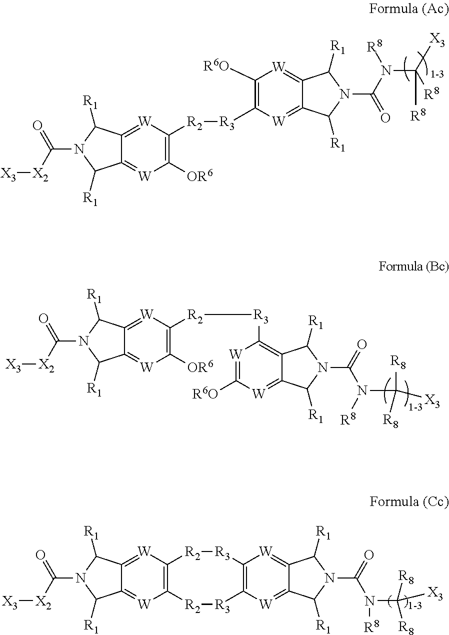

or a pharmaceutically acceptable salt thereof.

In some embodiments of the preceding Formulae defined herein, each heterocyclic ring group and each carbocyclic ring group includes single ring, spiral ring, bridge ring, fused ring and various combinations of spiral ring, bridge ring and/or fused ring.

In some embodiments of the preceding Formulae defined herein, the compound is selected from the compounds described in Table 1 and pharmaceutically acceptable salts thereof.

TABLE 1

| | |
|---|---|
| 1. | 4,4′-((propane-1,3-diylbis(oxy))bis(6-methoxyisoindoline-5,2- |
| | diyl))bis(4-oxobutanoic acid) |
| 2. | 4-(6-(3-((2-(3-carboxypropanoyl)-6-methoxyisoindolin-5-yl) |
| | oxy)propoxy)-4-fluoro-5-methoxyisoindolin-2-yl)-4- |
| | oxobutanoic acid |
| 3. | sodium (S)-4-(5-(3-((2-(3-carboxylatopropanoyl)-6- |
| | methoxyisoindolin-5-yl)oxy)propoxy)-6-methoxyisoindolin-2- |
| | yl)-2-methyl-4-oxobutanoate |
| 4. | 4-(5-(3-((2-(3-carboxypropanoyl)-4-fluoro-6- |
| | methoxyisoindolin-5-yl)oxy)propoxy)-6-methoxyisoindolin-2- |
| | yl)-4-oxobutanoic acid |
| 5. | 4,4′-((propane-1,3-diylbis(oxy))bis(3-methoxy-5,7-dihydro- |
| | 6H-pyrrolo[3,4-b]pyridine-2,6-diyl))bis(4-oxobutanoic acid) |
| 6. | 4-(5-(3-((2-(4-ethoxy-4-oxobutanoyl)-6-methoxyisoindolin-5- |
| | yl)oxy)propoxy)-6-methoxyisoindolin-2-yl)-4-oxobutanoic |
| | acid |
| 7. | 4,4′-(((methylazanediyl)bis(methylene))bis(6- |
| | methoxyisoindoline-5,2-diyl))bis(4-oxobutanoic acid) |
| 8. | 4-(5-(3-((2-(3-carboxypropanoyl)-4-chloro-6- |
| | methoxyisoindolin-5-yl)oxy)propoxy)-6-methoxyisoindolin-2- |
| | yl)-4-oxobutanoic acid |
| 9. | 4,4′-((propane-1,3-diylbis(oxy))bis(4-fluoro-6- |
| | methoxyisoindoline-5,2-diyl))bis(4-oxobutanoic acid) |
| 10. | 4,4′-(((2-methylenepropane-1,3-diyl)bis(oxy))bis(6- |
| | methoxyisoindoline-5,2-diyl))bis(4-oxobutanoic acid) |
| 11. | 4,4′-(((2-methylenepropane-1,3-diyl)bis(oxy))bis(4-chloro-6- |
| | methoxyisoindoline-5,2-diyl))bis(4-oxobutanoic acid) |
| 12. | 4,4′-((propane-1,3-diylbis(oxy))bis(4-chloro-6- |
| | methoxyisoindoline-5,2-diyl))bis(4-oxobutanoic acid) |
| 13. | 4-(5-(3-((2-(3-carboxypropanoyl)-4-chloro-6- |
| | methoxyisoindolin-5-yl)oxy)propoxy)-4-fluoro-6- |
| | methoxyisoindolin-2-yl)-4-oxobutanoic acid |
| 14. | 4-(5-(3-((2-((2-carboxyethyl)carbamoyl)-6-methoxyisoindolin- |
| | 5-yl)oxy)propoxy)-6-methoxyisoindolin-2-yl)-4-oxobutanoic |
| | acid |
| 15. | 4,4′-((propane-1,3-diylbis(oxy))bis(6-methoxyisoindoline-5,2- |
| | diyl))bis(2-methyl-4-oxobutanoic acid) |
| 16. | (2S,2′S)-4,4′-((propane-1,3-diylbis(oxy))bis(6- |
| | methoxyisoindoline-5,2-diyl))bis(2-methyl-4-oxobutanoic acid) |
| 17. | (S)-4-(5-(3-((2-(3-carboxypropanoyl)-6-methoxyisoindolin-5- |
| | yl)oxy)propoxy)-6-methoxyisoindolin-2-yl)-2-methyl-4- |
| | oxobutanoic acid |
| 18. | (2S,2′S)-4,4′-((ethane-1,2-diylbis(oxy))bis(6- |
| | methoxyisoindoline-5,2-diyl))bis(2-methyl-4-oxobutanoic acid) |
| 19. | (2S,2′S)-4,4′-((butane-1,4-diylbis(oxy))bis(6- |
| | methoxyisoindoline-5,2-diyl))bis(2-methyl-4-oxobutanoic acid) |
| 20. | (2S,2′S)-4,4′-((pentane-1,5-diylbis(oxy))bis(6- |
| | methoxyisoindoline-5,2-diyl))bis(2-methyl-4-oxobutanoic acid) |
| 21. | (S)-4-(5-(3-((2-((S)-3-carboxybutanoyl)-6-methoxyisoindolin- |
| | 5-yl)oxy)propyl)-6-methoxyisoindolin-2-yl)-2-methyl-4- |
| | oxobutanoic acid |
| 22. | (S)-4-(5-(3-((2-((S)-3-carboxybutanoyl)-6-methoxyisoindolin- |
| | 5-yl)amino)propoxy)-6-methoxyisoindolin-2-yl)-2-methyl-4- |
| | oxobutanoic acid |
| 23. | (S)-4-(5-(4-((2-((S)-3-carboxybutanoyl)-6-methoxyisoindolin- |
| | 5-yl)amino)butyl)-6-methoxyisoindolin-2-yl)-2-methyl-4- |
| | oxobutanoic acid |
| 24. | (2S,2′S)-4,4′-(propane-1,3-diylbis(6-methoxyisoindoline-5,2- |
| | diyl))bis(2-methyl-4-oxobutanoic acid) |
| 25. | (2S,2′S)-4,4′-(pentane-1,5-diylbis(6-methoxyisoindoline-5,2- |
| | diyl))bis(2-methyl-4-oxobutanoic acid) |
| 26. | (2S,2′S)-4,4′-((pentane-2,4-diylbis(oxy))bis(6- |
| | methoxyisoindoline-5,2-diyl))bis(2-methyl-4-oxobutanoic acid) |
| 27. | (2S,2′S)-4,4′-(((2,2-dimethylpropane-1,3-diyl)bis(oxy))bis(6- |
| | methoxyisoindoline-5,2-diyl))bis(2-methyl-4-oxobutanoic acid) |
| 28. | (2S,2′S)-4,4′-(((cyclopropane-1,1-diylbis(methylene))bis(oxy)) |
| | bis(6-methoxyisoindoline-5,2-diyl))bis(2-methyl-4-oxobutanoic |
| | acid) |
| 29. | (S)-4-(5-(3-((2-((S)-3-carboxybutanoyl)-6-methoxy-4- |
| | methylisoindolin-5-yl)oxy)propoxy)-6-methoxyisoindolin-2- |
| | yl)-2-methyl-4-oxobutanoic acid |
| 30. | (S)-4-(5-(3-((2-((S)-3-carboxybutanoyl)-4-fluoro-6- |
| | methoxyisoindolin-5-yl)oxy)propoxy)-6-methoxyisoindolin-2- |
| | yl)-2-methyl-4-oxobutanoic acid |
| 31. | (S)-4-(5-(3-((2-((S)-3-carboxybutanoyl)-4-chloro-6- |
| | methoxyisoindolin-5-yl)oxy)propoxy)-6-methoxyisoindolin-2- |
| | yl)-2-methyl-4-oxobutanoic acid |
| 32. | (S)-4-(5-(3-((4-bromo-2-((S)-3-carboxybutanoyl)-6- |
| | methoxyisoindolin-5-yl)oxy)propoxy)-6-methoxyisoindolin-2- |
| | yl)-2-methyl-4-oxobutanoic acid |
| 33. | (S)-4-(5-(3-((2-((S)-3-carboxybutanoyl)-6-methoxy-4,7- |
| | dimethylisoindolin-5-yl)oxy)propoxy)-6-methoxyisoindolin-2- |
| | yl)-2-methyl-4-oxobutanoic acid |
| 34. | (S)-4-(5-(3-((2-((S)-3-carboxybutanoyl)-6-methoxyisoindolin- |
| | 5-yl)oxy)propoxy)-7-fluoro-6-methoxy-4-methylisoindolin-2- |
| | yl)-2-methyl-4-oxobutanoic acid |
| 35. | (S)-4-(5-(3-((2-((S)-3-carboxybutanoyl)-6-methoxyisoindolin- |
| | 5-yl)oxy)propoxy)-7-chloro-6-methoxy-4-methylisoindolin-2- |
| | yl)-2-methyl-4-oxobutanoic acid |
| 36. | (S)-4-(5-(3-((7-bromo-2-((S)-3-carboxybutanoyl)-6-methoxy- |
| | 4-methylisoindolin-5-yl)oxy)propoxy)-6-methoxyisoindolin-2- |
| | yl)-2-methyl-4-oxobutanoic acid |
| 37. | (S)-4-(5-(3-((2-((S)-3-carboxybutanoyl)-4,7-difluoro-6- |
| | methoxyisoindolin-5-yl)oxy)propoxy)-6-methoxyisoindolin-2- |
| | yl)-2-methyl-4-oxobutanoic acid |
| 38. | (S)-4-(5-(3-((2-((S)-3-carboxybutanoyl)-6-methoxyisoindolin- |
| | 5-yl)oxy)propoxy)-7-chloro-4-fluoro-6-methoxyisoindolin-2- |
| | yl)-2-methyl-4-oxobutanoic acid |
| 39. | (S)-4-(5-(3-((7-bromo-2-((S)-3-carboxybutanoyl)-4-fluoro-6- |
| | methoxyisoindolin-5-yl)oxy)propoxy)-6-methoxyisoindolin-2- |
| | yl)-2-methyl-4-oxobutanoic acid |
| 40. | (S)-4-(5-(3-((2-((S)-3-carboxybutanoyl)-4-fluoro-6-methoxy- |
| | 7-methylisoindolin-5-yl)oxy)propoxy)-6-methoxyisoindolin-2- |
| | yl)-2-methyl-4-oxobutanoic acid |
| 41. | (S)-4-(5-(3-((2-((S)-3-carboxybutanoyl)-4-chloro-6-methoxy- |
| | 7-methylisoindolin-5-yl)oxy)propoxy)-6-methoxyisoindolin-2- |
| | yl)-2-methyl-4-oxobutanoic acid |
| 42. | (S)-4-(5-(3-((2-((S)-3-carboxybutanoyl)-4-chloro-7-fluoro-6- |
| | methoxyisoindolin-5-yl)oxy)propoxy)-6-methoxyisoindolin-2- |
| | yl)-2-methyl-4-oxobutanoic acid |
| 43. | (S)-4-(5-(3-((2-((S)-3-carboxybutanoyl)-4,7-dichloro-6- |
| | methoxyisoindolin-5-yl)oxy)propoxy)-6-methoxyisoindolin-2- |
| | yl)-2-methyl-4-oxobutanoic acid |
| 44. | (S)-4-(5-(3-((7-bromo-2-((S)-3-carboxybutanoyl)-4-chloro-6- |
| | methoxyisoindolin-5-yl)oxy)propoxy)-6-methoxyisoindolin-2- |
| | yl)-2-methyl-4-oxobutanoic acid |
| 45. | (S)-4-(5-(3-((4-bromo-2-((S)-3-carboxybutanoyl)-6-methoxy- |
| | 7-methylisoindolin-5-yl)oxy)propoxy)-6-methoxyisoindolin-2- |
| | yl)-2-methyl-4-oxobutanoic acid |
| 46. | (S)-4-(5-(3-((4-bromo-2-((S)-3-carboxybutanoyl)-7-fluoro-6- |
| | methoxyisoindolin-5-yl)oxy)propoxy)-6-methoxyisoindolin-2- |
| | yl)-2-methyl-4-oxobutanoic acid |
| 47. | (S)-4-(5-(3-((4-bromo-2-((S)-3-carboxybutanoyl)-7-chloro-6- |
| | methoxyisoindolin-5-yl)oxy)propoxy)-6-methoxyisoindolin-2- |
| | yl)-2-methyl-4-oxobutanoic acid |
| 48. | (S)-4-(5-(3-((4,7-dibromo-2-((S)-3-carboxybutanoyl)-6- |
| | methoxyisoindolin-5-yl)oxy)propoxy)-6-methoxyisoindolin-2- |
| | yl)-2-methyl-4-oxobutanoic acid |
| 49. | (2S)-4-(5-(3-((2-((S)-3-carboxybutanoyl)-6-methoxy-3,4- |
| | dimethylisoindolin-5-yl)oxy)propoxy)-6-methoxyisoindolin-2- |
| | yl)-2-methyl-4-oxobutanoic acid |
| 50. | (2S)-4-(5-(3-((2-((S)-3-carboxybutanoyl)-4-fluoro-6-methoxy- |
| | 3-methylisoindolin-5-yl)oxy)propoxy)-6-methoxyisoindolin-2- |
| | yl)-2-methyl-4-oxobutanoic acid |
| 51. | (2S)-4-(5-(3-((2-((S)-3-carboxybutanoyl)-4-chloro-6-methoxy- |
| | 3-methylisoindolin-5-yl)oxy)propoxy)-6-methoxyisoindolin-2- |
| | yl)-2-methyl-4-oxobutanoic acid |
| 52. | (2S)-4-(5-(3-((4-bromo-2-((S)-3-carboxybutanoyl)-6-methoxy- |
| | 3-methylisoindolin-5-yl)oxy)propoxy)-6-methoxyisoindolin-2- |
| | yl)-2-methyl-4-oxobutanoic acid |
| 53. | (2S)-4-(5-(3-((2-((S)-3-carboxybutanoyl)-6-methoxy-3,4,7- |
| | trimethylisoindolin-5-yl)oxy)propoxy)-6-methoxyisoindolin-2- |
| | yl)-2-methyl-4-oxobutanoic acid |
| 54 | (2S)-4-(6-(3-((2-((S)-3-carboxybutanoyl)-6-methoxyisoindolin- |
| | 5-yl)oxy)propoxy)-4-fluoro-5-methoxy-1,7-dimethylisoindolin- |
| | 2-yl)-2-methyl-4-oxobutanoic acid |
| 55. | (2S)-4-(6-(3-((2-((S)-3-carboxybutanoyl)-6-methoxyisoindolin- |
| | 5-yl)oxy)propoxy)-4-chloro-5-methoxy-1,7-dimethylisoindolin- |
| | 2-yl)-2-methyl-4-oxobutanoic acid |
| 56. | (2S)-4-(5-(3-((7-bromo-2-((S)-3-carboxybutanoyl)-6-methoxy- |
| | 3,4-dimethylisoindolin-5-yl)oxy)propoxy)-6- |
| | methoxyisoindolin-2-yl)-2-methyl-4-oxobutanoic acid |
| 57. | (2S)-4-(5-(3-((2-((S)-3-carboxybutanoyl)-4,7-difluoro-6- |
| | methoxy-3-methylisoindolin-5-yl)oxy)propoxy)-6- |
| | methoxyisoindolin-2-yl)-2-methyl-4-oxobutanoic acid |
| 58. | (2S)-4-(6-(3-((2-((S)-3-carboxybutanoyl)-6-methoxyisoindolin- |
| | 5-yl)oxy)propoxy)-4-chloro-7-fluoro-5-methoxy-1- |
| | methylisoindolin-2-yl)-2-methyl-4-oxobutanoic acid |
| 59. | (2S)-4-(5-(3-((7-bromo-2-((S)-3-carboxybutanoyl)-4-fluoro-6- |
| | methoxy-3-methylisoindolin-5-yl)oxy)propoxy)-6- |
| | methoxyisoindolin-2-yl)-2-methyl-4-oxobutanoic acid |
| 60. | (2S)-4-(5-(3-((2-((S)-3-carboxybutanoyl)-4-fluoro-6-methoxy- |
| | 3,7-dimethylisoindolin-5-yl)oxy)propoxy)-6- |
| | methoxyisoindolin-2-yl)-2-methyl-4-oxobutanoic acid |
| 61 | (2S)-4-(5-(3-((2-((S)-3-carboxybutanoyl)-4-chloro-6-methoxy- |
| | 3,7-dimethylisoindolin-5-yl)oxy)propoxy)-6- |
| | methoxyisoindolin-2-yl)-2-methyl-4-oxobutanoic acid |
| 62. | (2S)-4-(5-(3-((2-((S)-3-carboxybutanoyl)-4-chloro-7-fluoro-6- |
| | methoxy-3-methylisoindolin-5-yl)oxy)propoxy)-6- |
| | methoxyisoindolin-2-yl)-2-methyl-4-oxobutanoic acid |
| 63. | (2S)-4-(5-(3-((2-((S)-3-carboxybutanoyl)-4,7-dichloro-6- |
| | methoxy-3-methylisoindolin-5-yl)oxy)propoxy)-6- |
| | methoxyisoindolin-2-yl)-2-methyl-4-oxobutanoic acid |
| 64. | (2S)-4-(5-(3-((7-bromo-2-((S)-3-carboxybutanoyl)-4-chloro-6- |
| | methoxy-3-methylisoindolin-5-yl)oxy)propoxy)-6- |
| | methoxyisoindolin-2-yl)-2-methyl-4-oxobutanoic acid |
| 65. | (2S)-4-(5-(3-((4-bromo-2-((S)-3-carboxybutanoyl)-6-methoxy- |
| | 3,7-dimethylisoindolin-5-yl)oxy)propoxy)-6- |
| | methoxyisoindolin-2-yl)-2-methyl-4-oxobutanoic acid |
| 66. | (2S)-4-(5-(3-((4-bromo-2-((S)-3-carboxybutanoyl)-7-fluoro-6- |
| | methoxy-3-methylisoindolin-5-yl)oxy)propoxy)-6- |
| | methoxyisoindolin-2-yl)-2-methyl-4-oxobutanoic acid |
| 67 | (2S)-4-(5-(3-((4-bromo-2-((S)-3-carboxybutanoyl)-7-chloro-6- |
| | methoxy-3-methylisoindolin-5-yl)oxy)propoxy)-6- |
| | methoxyisoindolin-2-yl)-2-methyl-4-oxobutanoic acid |
| 68 | (2S)-4-(5-(3-((4,7-dibromo-2-((S)-3-carboxybutanoyl)-6- |
| | methoxy-3-methylisoindolin-5-yl)oxy)propoxy)-6- |
| | methoxyisoindolin-2-yl)-2-methyl-4-oxobutanoic acid |
| 69. | (2S)-4-(6-(3-((2-((S)-3-carboxybutanoyl)-4-fluoro-6- |
| | methoxyisoindolin-5-yl)oxy)propoxy)-5-methoxy-1- |
| | methylisoindolin-2-yl)-2-methyl-4-oxobutanoic acid |
| 70. | (2S,2′S)-4,4′-((propane-1,3-diylbis(oxy))bis(4-fluoro-6- |
| | methoxyisoindoline-5,2-diyl))bis(2-methyl-4-oxobutanoic acid) |
| 71. | (S)-4-(5-(3-((2-((S)-3-carboxybutanoyl)-4-chloro-6- |
| | methoxyisoindolin-5-yl)oxy)propoxy)-4-fluoro-6- |
| | methoxyisoindolin-2-yl)-2-methyl-4-oxobutanoic acid |
| 72. | (S)-4-(6-(3-((2-((S)-3-carboxybutanoyl)-4-fluoro-6- |
| | methoxyisoindolin-5-yl)oxy)propoxy)-4-chloro-5- |
| | methoxyisoindolin-2-yl)-2-methyl-4-oxobutanoic acid |
| 73. | (S)-4-(6-(3-((2-((S)-3-carboxybutanoyl)-4-chloro-6- |
| | methoxyisoindolin-5-yl)oxy)propoxy)-4-chloro-5- |
| | methoxyisoindolin-2-yl)-2-methyl-4-oxobutanoic acid |
| 74. | (S)-4-(6-(3-((2-((S)-3-carboxybutanoyl)-4-chloro-6- |
| | methoxyisoindolin-5-yl)oxy)propoxy)-4-fluoro-5- |
| | methoxyisoindolin-2-yl)-2-methyl-4-oxobutanoic acid |
| 75. | (S)-4-(6-(3-((2-((S)-3-carboxybutanoyl)-6-methoxyisoindolin- |
| | 5-yl)oxy)propoxy)-4-chloro-5-methoxyisoindolin-2-yl)-2- |
| | methyl-4-oxobutanoic acid |
| 76. | (S)-4-(6-(3-((2-((S)-3-carboxybutanoyl)-6-methoxyisoindolin- |
| | 5-yl)oxy)propoxy)-4-fluoro-5-methoxyisoindolin-2-yl)-2- |
| | methyl-4-oxobutanoic acid |
| 77. | (S)-4-(5-(3-((6-((S)-3-carboxybutanoyl)-3-methoxy-6,7- |
| | dihydro-5H-pyrrolo[3,4-b]pyridin-2-yl)oxy)propoxy)-6- |
| | methoxyisoindolin-2-yl)-2-methyl-4-oxobutanoic acid |
| 78. | (S)-4-(5-(3-((6-((S)-3-carboxybutanoyl)-3-methoxy-6,7- |
| | dihydro-5H-pyrrolo[3,4-b]pyrazin-2-yl)oxy)propoxy)-6- |
| | methoxyisoindolin-2-yl)-2-methyl-4-oxobutanoic acid |
| 79. | (2S,2′S)-4,4′-((propane-1,3-diylbis(oxy))bis(3-methoxy-5,7- |
| | dihydro-6H-pyrrolo[3,4-b]pyridine-2,6-diyl))bis(2-methyl-4- |
| | oxobutanoic acid) |
| 80. | (S)-4-(2-(3-((6-((S)-3-carboxybutanoyl)-2-methoxy-6,7- |
| | dihydro-5H-pyrrolo[3,4-b]pyridin-3-yl)oxy)propoxy)-3- |
| | methoxy-5,7-dihydro-6H-pyrrolo[3,4-b]pyridin-6-yl)-2- |
| | methyl-4-oxobutanoic acid |
| 81. | (2S,2′S)-4,4′-((propane-1,3-diylbis(oxy))bis(2-methoxy-5,7- |
| | dihydro-6H-pyrrolo[3,4-b]pyridine-3,6-diyl))bis(2-methyl-4- |
| | oxobutanoic acid) |
| 82. | (S)-4-(5-(3-((6-((S)-3-carboxybutanoyl)-2-methoxy-6,7- |
| | dihydro-5H-pyrrolo[3,4-b]pyridin-3-yl)oxy)propoxy)-6- |
| | methoxyisoindolin-2-yl)-2-methyl-4-oxobutanoic acid |
| 83. | (2S,2′S)-4,4′-(1,3,7,8,11,13,17,18-octahydro-2H,6H,12H,16H- |
| | [1,4,8,11]tetraoxacyclotetradecino[2,3-f:9,10-f′]diisoindole- |
| | 2,12-diyl)bis(2-methyl-4-oxobutanoic acid) |
| 84. | (S)-4-(5-methoxy-6-(3-((6-methoxy-2-((S)-3-methyl-4- |
| | (methylsulfonamido)-4-oxobutanoyl)isoindolin-5- |
| | yl)oxy)propoxy)isoindolin-2-yl)-2-methyl-4-oxobutanoic acid |
| 85. | (S)-4-(5-(3-((2-((S)-4-((N,N-dimethylsulfamoyl)amino)-3- |
| | methyl-4-oxobutanoyl)-6-methoxyisoindolin-5- |
| | yl)oxy)propoxy)-6-methoxyisoindolin-2-yl)-2-methyl-4- |
| | oxobutanoic acid |
| 86. | 4,4′-((propane-1,3-diylbis(oxy))bis(6-methoxyisoindoline-5,2- |
| | diyl))bis(4-oxobutanenitrile) |
| 87. | (S)-4-(5-(3-((2-(3-cyanopropanoyl)-6-methoxyisoindolin-5- |
| | yl)oxy)propoxy)-6-methoxyisoindolin-2-yl)-2-methyl-4- |
| | oxobutanoic acid |
| 88. | (1R,2R)-2-(5-(3-((2-((S)-3-carboxybutanoyl)-6- |
| | methoxyisoindolin-5-yl)oxy)propoxy)-6-methoxyisoindoline- |
| | 2-carbonyl)cyclobutane-1-carboxylic acid |
| 89. | (1R,2R)-2-(5-(3-((2-((S)-3-carboxybutanoyl)-6- |
| | methoxyisoindolin-5-yl)oxy)propoxy)-6-methoxyisoindoline- |
| | 2-carbonyl)cyclopropane-1-carboxylic acid |
| 90. | (1S,2S)-2-(5-(3-((2-((1R,2R)-2-carboxycyclopropane-1- |
| | carbonyl)-6-methoxyisoindolin-5-yl)oxy)propoxy)-6- |
| | methoxyisoindoline-2-carbonyl)cyclopropane-1-carboxylic |
| | acid |
| 91. | (S)-4-(5-(3-((2-((S)-3-carboxybutanoyl)-6-methoxyisoindolin- |
| | 4-yl)oxy)propoxy)-6-methoxyisoindolin-2-yl)-2-methyl-4- |
| | oxobutanoic acid |
| 92. | (S)-4-(5-(4-(2-((S)-3-carboxybutanoyl)-6-methoxyisoindolin-4- |
| | yl)butoxy)-6-methoxyisoindolin-2-yl)-2-methyl-4-oxobutanoic |
| | acid |
| 93. | (S)-4-(5-(4-((2-((S)-3-carboxybutanoyl)-6-methoxyisoindolin- |
| | 4-yl)oxy)butyl)-6-methoxyisoindolin-2-yl)-2-methyl-4- |
| | oxobutanoic acid |
| 94 | (S)-4-(5-(3-((2-((S)-3-carboxybutanoyl)-6-methoxyisoindolin- |
| | 5-yl)oxy)propoxy)-6-methylisoindolin-2-yl)-2-methyl-4- |
| | oxobutanoic acid |
| 95. | (S)-4-(5-(3-((2-((S)-3-carboxybutanoyl)-6-ethylisoindolin-5- |
| | yl)oxy)propoxy)-6-methoxyisoindolin-2-yl)-2-methyl-4- |
| | oxobutanoic acid |
| 96. | (S)-4-(5-(3-((2-((S)-3-carboxybutanoyl)-6-methoxyisoindolin- |
| | 5-yl)oxy)propoxy)-6-vinylisoindolin-2-yl)-2-methyl-4- |
| | oxobutanoic acid |
| 97. | (S)-4-(5-(3-((2-((S)-3-carboxybutanoyl)-6-ethynylisoindolin- |
| | 5-yl)oxy)propoxy)-6-methoxyisoindolin-2-yl)-2-methyl-4- |
| | oxobutanoic acid |
| 98. | (S)-4-(5-(3-((6-amino-2-((S)-3-carboxybutanoyl)isoindolin-5- |
| | yl)oxy)propoxy)-6-methoxyisoindolin-2-yl)-2-methyl-4- |
| | oxobutanoic acid |
| 99. | (S)-4-(5-(3-((2-((S)-3-carboxybutanoyl)-6-(methylamino) |
| | isoindolin-5-yl)oxy)propoxy)-6-methoxyisoindolin-2-yl)-2- |
| | methyl-4-oxobutanoic acid |
| 100. | (S)-4-(5-(3-((6-bromo-2-((S)-3-carboxybutanoyl)isoindolin-5- |
| | yl)oxy)propoxy)-6-methoxyisoindolin-2-yl)-2-methyl-4- |
| | oxobutanoic acid |
| 101. | (S)-4-(5-(3-((2-((S)-3-carboxybutanoyl)-4-fluoro-6- |
| | methoxyisoindolin-5-yl)oxy)propoxy)-6-hydroxyisoindolin-2- |
| | yl)-2-methyl-4-oxobutanoic acid |
| 102. | (S)-4-(5-(3-((2-((S)-3-carboxybutanoyl)-4-chloro-6- |
| | methoxyisoindolin-5-yl)oxy)propoxy)-6-hydroxyisoindolin-2- |
| | yl)-2-methyl-4-oxobutanoic acid |
| 103. | (S)-4-(5-(3-((2-((S)-3-carboxybutanoyl)-4-fluoro-6- |
| | hydroxyisoindolin-5-yl)oxy)propoxy)-4-fluoro-6- |
| | methoxyisoindolin-2-yl)-2-methyl-4-oxobutanoic acid |
| 104. | (S)-4-(6-(3-((2-((S)-3-carboxybutanoyl)-4-fluoro-6- |
| | hydroxyisoindolin-5-yl)oxy)propoxy)-4-fluoro-5- |
| | methoxyisoindolin-2-yl)-2-methyl-4-oxobutanoic acid |
| 105. | (S)-4-(5-(3-((2-((S)-3-carboxybutanoyl)-4-chloro-6- |
| | hydroxyisoindolin-5-yl)oxy)propoxy)-4-fluoro-6- |
| | methoxyisoindolin-2-yl)-2-methyl-4-oxobutanoic acid |
| 106. | (S)-4-(5-(3-((2-((S)-3-carboxybutanoyl)-4-chloro-6- |
| | methoxyisoindolin-5-yl)oxy)propoxy)-4-fluoro-6- |
| | hydroxyisoindolin-2-yl)-2-methyl-4-oxobutanoic acid |
| 107. | (S)-4-(5-(3-((2-((S)-3-carboxybutanoyl)-4-fluoro-6- |
| | hydroxyisoindolin-5-yl)oxy)propoxy)-6-methoxyisoindolin-2- |
| | yl)-2-methyl-4-oxobutanoic acid |
| 108. | 4,4′-((azanediylbis(methylene))bis(6-methoxyisoindoline-5,2- |
| | diyl))bis(4-oxobutanoic acid) |
| 109. | 4,4′-((propane-1,3-diylbis(oxy))bis(2-methoxy-5,7-dihydro- |
| | 6H-pyrrolo[3,4-b]pyridine-3,6-diyl))bis(4-oxobutanoic acid) |
| 110. | 4-(2-(3-((6-(3-carboxypropanoyl)-2-methoxy-6,7-dihydro-5H- |
| | pyrrolo[3,4-b]pyridin-3-yl)oxy)propoxy)-3-methoxy-5,7- |
| | dihydro-6H-pyrrolo[3,4-b]pyridin-6-yl)-4-oxobutanoic acid |
| 111. | 4-(5-(3-((6-(3-carboxypropanoyl)-3-methoxy-6,7-dihydro-5H- |
| | pyrrolo[3,4-b]pyridin-2-yl)oxy)propoxy)-6-methoxyisoindolin- |
| | 2-yl)-4-oxobutanoic acid |
| 112. | 4-(5-(3-((6-(3-carboxypropanoyl)-2-methoxy-6,7-dihydro-5H- |
| | pyrrolo[3,4-b]pyridin-3-yl)oxy)propoxy)-6-methoxyisoindolin- |
| | 2-yl)-4-oxobutanoic acid |
| 113. | 4-(2-(3-((6-(4-(((4R,5R)-5-hydroxy-1,2-dithian-4-yl)oxy)-4- |
| | oxobutanoyl)-3-methoxy-6,7-dihydro-5H-pyrrolo[3,4-b] |
| | pyridin-2-yl)oxy)propoxy)-3-methoxy-5,7-dihydro-6H- |
| | pyrrolo[3,4-b]pyridin-6-yl)-4-oxobutanoic acid |
| 114. | 4-(5-((2-(((2-(3-carboxypropanoyl)-6-methoxy-2H-isoindol-5- |
| | yl)oxy)methyl)allyl)oxy)-6-methoxyisoindolin-2-yl)-4- |
| | oxobutanoic acid |
| 115. | 4-(5-((2-(((2-(3-carboxypropanoyl)-6-methoxy-1H-indol-5-yl) |
| | oxy)methyl)allyl)oxy)-6-methoxyisoindolin-2-yl)-4- |
| | oxobutanoic acid |
| 116. | 4-(5-((2-(((2-(3-carboxypropanoyl)-6-methoxy-1H-inden-5-yl) |
| | oxy)methyl)allyl)oxy)-6-methoxyisoindolin-2-yl)-4- |
| | oxobutanoic acid |
| 117. | 4-(5-(3-((2-(3-carboxypropanoyl)-6-methoxy-2H-isoindol-5- |
| | yl)oxy)propoxy)-6-methoxyisoindolin-2-yl)-4-oxobutanoic |
| | acid |
| 118. | 4-(5-(3-((2-(3-carboxypropanoyl)-6-methoxy-1H-indol-5-yl) |
| | oxy)propoxy)-6-methoxyisoindolin-2-yl)-4-oxobutanoic acid |
| 119 | 4-(5-(3-((2-(3-carboxypropanoyl)-6-methoxy-1H-inden-5-yl) |
| | oxy)propoxy)-6-methoxyisoindolin-2-yl)-4-oxobutanoic acid |
| 120. | 4-(5-(3-((2-(3-carboxypropanoyl)-4-fluoro-6- |
| | methoxyisoindolin-5-yl)oxy)propoxy)-6-methoxy-2H-isoindol- |
| | 2-yl)-4-oxobutanoic acid |
| 121. | 4-(5-(3-((2-(3-carboxypropanoyl)-4-fluoro-6- |
| | methoxyisoindolin-5-yl)oxy)propoxy)-6-methoxy-1H-indol-2- |
| | yl)-4-oxobutanoic acid |
| 122. | 4-(5-(3-((2-(3-carboxypropanoyl)-4-fluoro-6- |
| | methoxyisoindolin-5-yl)oxy)propoxy)-6-methoxy-1H-inden-2- |
| | yl)-4-oxobutanoic acid |
| 123. | 4-(5-(3-((2-(3-carboxypropanoyl)-4-fluoro-6-methoxy-2H- |
| | isoindol-5-yl)oxy)propoxy)-6-methoxyisoindolin-2-yl)-4- |
| | oxobutanoic acid |
| 124. | 4-(5-(3-((2-(3-carboxypropanoyl)-4-fluoro-6-methoxy-1H- |
| | indol-5-yl)oxy)propoxy)-6-methoxyisoindolin-2-yl)-4- |
| | oxobutanoic acid |
| 125. | 4-(5-(3-((2-(3-carboxypropanoyl)-4-fluoro-6-methoxy-1H- |
| | inden-5-yl)oxy)propoxy)-6-methoxyisoindolin-2-yl)-4- |
| | oxobutanoic acid |
| 126. | 4-(5-(3-((2-(3-carboxypropanoyl)-4-fluoro-6-methoxy-2H- |
| | isoindol-5-yl)oxy)propoxy)-4-fluoro-6-methoxyisoindolin-2- |
| | yl)-4-oxobutanoic acid |
| 127. | 4-(5-(3-((2-(3-carboxypropanoyl)-4-fluoro-6-methoxy-1H- |
| | indol-5-yl)oxy)propoxy)-4-fluoro-6-methoxyisoindolin-2-yl)- |
| | 4-oxobutanoic acid |
| 128. | 4-(5-(3-((2-(3-carboxypropanoyl)-4-fluoro-6-methoxy-1H- |
| | inden-5-yl)oxy)propoxy)-4-fluoro-6-methoxyisoindolin-2-yl)- |
| | 4-oxobutanoic acid |

In some embodiments, the STING agonist moiety [D] is a compound of Formula I, II, III, IV or V, or an ester, stereoisomer, tautomer, isotopic derivative, prodrug or pharmaceutically acceptable salt thereof:

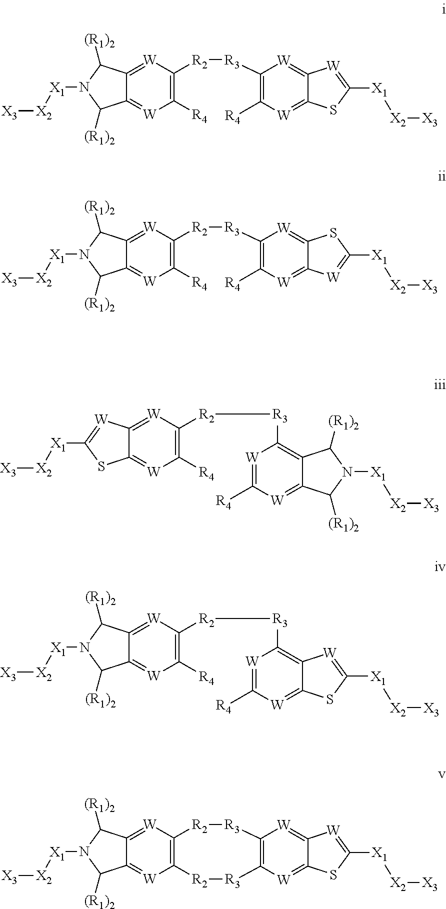

wherein
each W is independently selected from CR₁ or N;
each R₁ is independently selected from H, deuterium, halogen, OR⁶, N(R⁶)₂, COOR⁶, or C(O)N(R⁶)₂, CN or C₁-C₆ alkyl; wherein the C₁-C₆ alkyl is optionally substituted with one or more deuterium, halogen, OR⁶, N(R⁶)₂, COOR⁶, or C(O)N(R⁶)₂;
R₂ and R₃ are independently selected from O—(C₁-C₄ alkylene or haloalkylene), C₁-C₅ alkylene or haloalkylene, N(R⁶)—(C₁-C₄ alkylene or haloalkylene), -Tₐ-C₁-C₆ alkyl-T_{b}-, -Tₐ- N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}-, - Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃-C₁₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ- (C₃-C₁₂cycloalkyl)-T_{b}-, -Tₐ- (C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O— (C₁-C₆ alkyl)-O—; wherein the C₁-C₆ alkyl is optionally substituted with one or more halogen; and
each Rs independently is H, deuterium or C₁-C₆ alkyl optionally substituted with one or more halogen;
each R⁴ is independently selected from the group consisting of H, deuterium, halogen, CN, OR⁶, N(R⁶)₂, COOR⁶, C(O)N(R⁶)₂, SO₂R⁶, C₁-C₆ alkyl, C₁-C₆ haloalkyl, C₁-C₆ alkyl substituted by OR⁶, C₂-C₆ alkenyl, C₂-C₆ haloalkenyl, C₂-C₆ alkenyl substituted by OR⁶, C₂-C₆ alkynyl, C₂-C₆ haloalkynyl, C₂-C₆ alkynyl substituted by OR⁶, C₃-C₆ cycloalkyl, and a 3- to 6-membered heterocyclic ring including 1 to 2 ring members selected from the group consisting of O, S, and N(R⁶);
each R⁶ is independently selected from the group consisting of —H, deuterium, halogen, —NH₂, —CN, —OH, —N₃, —NO₂, carboxyl, C₁₋₆alkyl, C₁₋₆alkoxy, C₂₋₆alkenyl, C₂₋₆alkynyl, —C₆₋₁₀ aryl, —C₅₋₁₀heteroaryl, C₃₋₁₀heterocyclic ring or C₃₋₁₀carbocyclic ring; and each of which is independently optionally substituted with deuterium, halogen, —NH₂, —CN, —OH, —NO₂, carbonyl, ═O, oxo, carboxyl, C₁₋₆alkoxy, or C₁₋₆alkyl; and each of the heteroaryl and heterocyclic ring contains at least one heteroatoms selected from N, O or S;
each X₁ is independently selected from the group consisting of C═O, —CH₂—, —CHF—, and —CF₂—;
each X₂ is independently selected from (C(R⁸)₂)₍₁₋₃₎, —NR⁸(C(R⁸)₂)₍₁₋₃₎, —NH(C(R⁸)₂)₍₁₋₃₎, —N(C₁₋₆alkyl) (C(R⁸)₂)₍₁₋₃₎ or —N(haloC₁_6alkyl) (C(R⁸)₂)₍₁₋₃₎; wherein each R⁸ is independently selected from the group consisting of H, deuterium, halogen, C₁-C₆ alkyl, CN, OR⁶, N(R⁶)₂, C₁-C₆ haloalkyl, C₃-C₆ cycloalkyl, C₁-C₆ alkyl substituted by OR⁶, and C₁-C₆ alkyl substituted by N(R⁶)₂; optionally 2 R⁸ on different carbon atoms may be taken together, along with the atoms to which they are attached, to form a 3- to 6-membered fused ring; and optionally 2 R⁸ on a single carbon atom may be taken together, along with the atom to which they are attached, to form a 3- to 6-membered spirocycle;
each X₃ is independently selected from the group consisting of COOR⁶, C(O)SR⁶, C(S)OR⁶, SO₂R⁶, C(O)N(R⁹)₂,

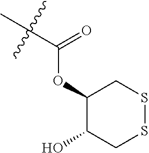 [SMILES: *C(=O)O[C@H]1CSSC[C@@H]1O]

and CN; and each R⁹ is independently selected from H, deuterium, COOR⁶, SO₂R⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆alkyl), S(C₆-C₁₀ aryl), S(═O)₂R⁶, S(═O)₂O R⁶, P(═O)(R⁶)₂, C₁-C₆alkyl, C₂-C₆ alkenyl, C₂-C₆ alkynyl, C₃-C₅ cycloalkyl, C₆-C₁₀aryl, 3-8 membered heterocycloalkyl, or 3-10 membered heteroaryl.

In some embodiments, the STING agonist moiety [D] is a compound of Formula I, II, III, IV or V, or an ester, stereoisomer, tautomer, isotopic derivative, prodrug or pharmaceutically acceptable salt thereof:

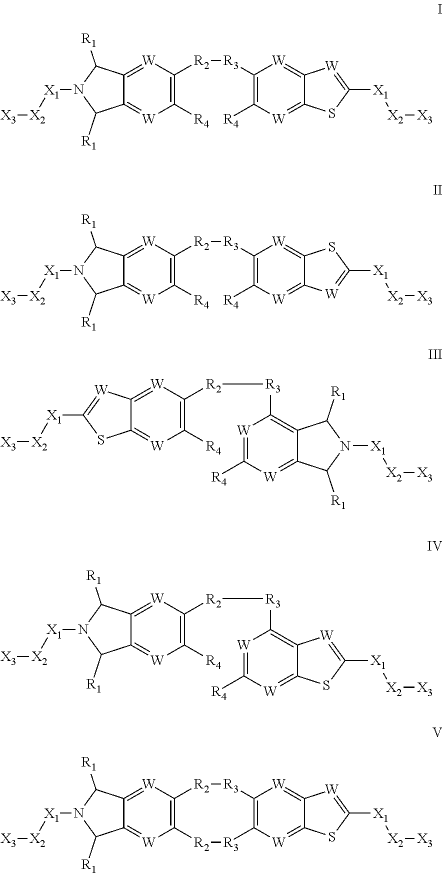

wherein
each W is independently selected from CR₁ or N;
each R₁ is independently selected from H, deuterium, halogen, OR⁶, N(R⁶)₂, COOR⁶, or C(O)N(R⁶)₂, CN or C₁-C₆ alkyl; wherein the C₁-C₆ alkyl is optionally substituted with one or more deuterium, halogen, OR⁶, N(R⁶)₂, COOR⁶, or C(O)N(R⁶)₂;
R₂ and R₃ are independently selected from O—(C₁-C₄ alkylene or haloalkylene), C₁-C₅ alkylene or haloalkylene, N(R⁶)—(C₁-C₄ alkylene or haloalkylene), -Tₐ-C₁-C₆ alkyl-T_{b}-, -Tₐ- N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}-, - Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃-C₁₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ- (C₃-C₁₂cycloalkyl)-T_{b}-, -Tₐ- (C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O— (C₁-C₆ alkyl)-O—; wherein the C₁-C₆ alkyl is optionally substituted with one or more halogen; and
each Rs independently is H, deuterium or C₁-C₆ alkyl optionally substituted with one or more halogen;
each R⁴ is independently selected from the group consisting of H, deuterium, halogen, CN, OR⁶, N(R⁶)₂, COOR⁶, C(O)N(R⁶)₂, SO₂R⁶, C₁-C₆ alkyl, C₁-C₆ haloalkyl, C₁-C₆ alkyl substituted by OR⁶, C₂-C₆ alkenyl, C₂-C₆ haloalkenyl, C₂-C₆ alkenyl substituted by OR⁶, C₂-C₆ alkynyl, C₂-C₆ haloalkynyl, C₂-C₆ alkynyl substituted by OR⁶, C₃-C₆ cycloalkyl, and a 3- to 6-membered heterocyclic ring including 1 to 2 ring members selected from the group consisting of O, S, and N(R⁶);
each R⁶ is independently selected from the group consisting of —H, deuterium, halogen, —NH₂, —CN, —OH, —N₃, —NO₂, carboxyl, C₁₋₆alkyl, C₁₋₆alkoxy, C_{2 6}alkenyl, C_{2 6}alkynyl, —C_{6 10}aryl, —C₅₋₁₀heteroaryl, C₃ ioheterocyclic ring or C₃₁ₗocarbocyclic ring; and each of which is independently optionally substituted with deuterium, halogen, —NH₂, —CN, —OH, —NO₂, carbonyl, ═O, oxo, carboxyl, C₁₋₆alkoxy, or C₁₋₆alkyl; and each of the heteroaryl and heterocyclic ring contains at least one heteroatoms selected from N, O or S;
each X₁ is independently selected from the group consisting of C═O, —CH₂—, —CHF—, and —CF₂—;
each X₂ is independently selected from (C(R⁸)₂)₍₁₋₃₎, —NR⁸(C(R⁸)₂)₍₁₋₃₎, —NH(C(R⁸)₂)₍₁₋₃₎, —N(C₁₋₆alkyl) (C(R⁸)₂)₍₁₋₃₎ or —N(haloC₁₋₆alkyl) (C(R⁸)₂)₍₁₋₃₎; wherein each R⁸ is independently selected from the group consisting of H, deuterium, halogen, C₁-C₆ alkyl, CN, OR⁶, N(R⁶)₂, C₁-C₆ haloalkyl, C₃-C₆ cycloalkyl, C₁-C₆ alkyl substituted by OR⁶, and C₁-C₆ alkyl substituted by N(R⁶)₂; optionally 2 R⁸ on different carbon atoms may be taken together, along with the atoms to which they are attached, to form a 3- to 6-membered fused ring; and optionally 2 R⁸ on a single carbon atom may be taken together, along with the atom to which they are attached, to form a 3- to 6-membered spirocycle;
each X₃ is independently selected from the group consisting of COOR⁶, C(O)SR⁶, C(S)OR⁶, SO₂R⁶, C(O)N(R⁹)₂,

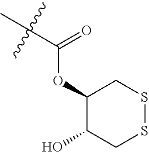 [SMILES: *C(=O)O[C@H]1CSSC[C@@H]1O]

and CN; and each R⁹ is independently selected from H, deuterium, COOR⁶, SO₂R⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆alkyl), S(C₆-C₁₀ aryl), S(═O)₂R⁶, S(═O)₂O R⁶, P(═O)(R⁶)₂, C₁-C₆alkyl, C₂-C₆ alkenyl, C₂-C₆ alkynyl, C₃-C₅ cycloalkyl, C₆-C₁₀aryl, 3-8 membered heterocycloalkyl, or 3-10 membered heteroaryl.

In some embodiments of Formula I,

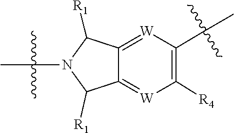

is independently selected from

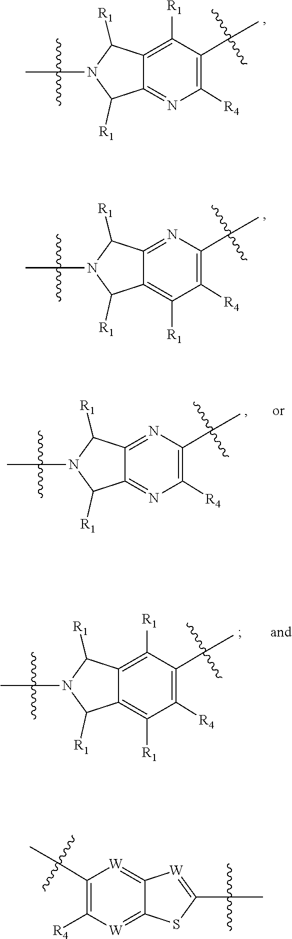

is independently selected from

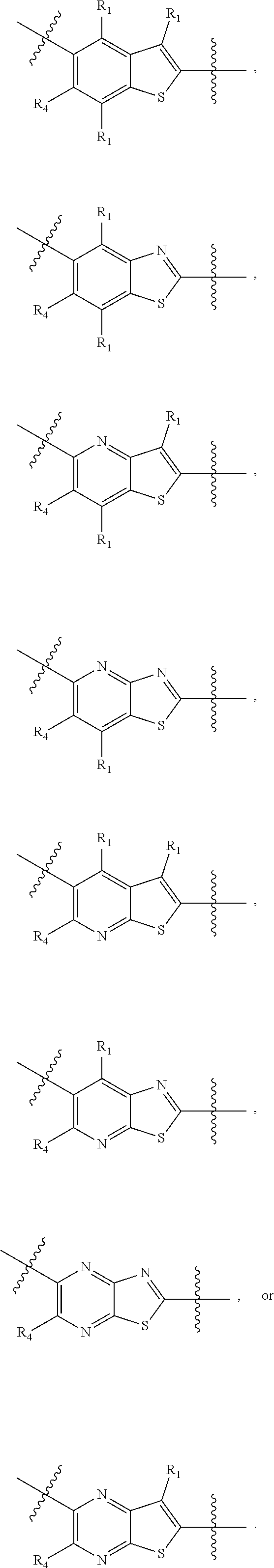

In some embodiments of Formula I,

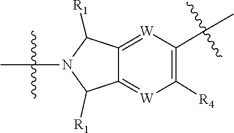

is independently selected from

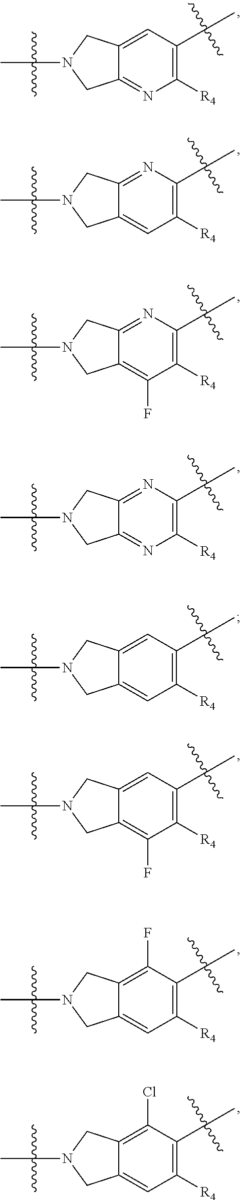

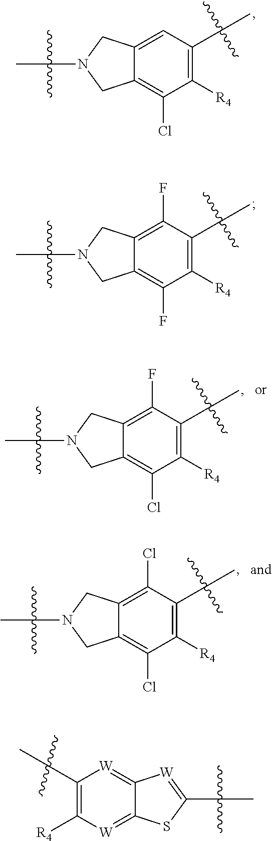

is independently selected from

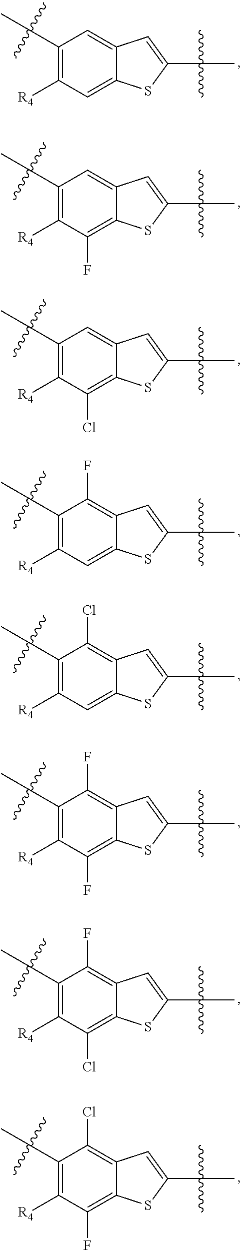

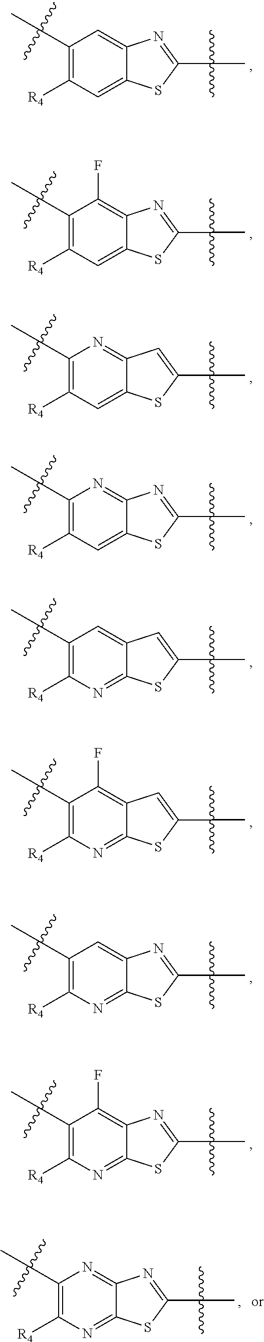

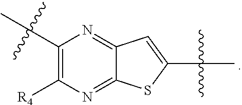

In some embodiments of Formula I, the compound is of Formula I-1:

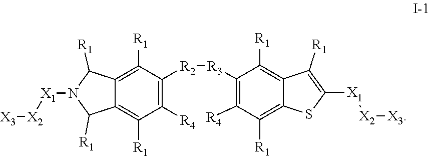

In some embodiments of Formula II,

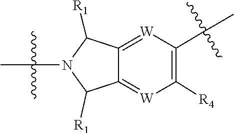

is independently selected from

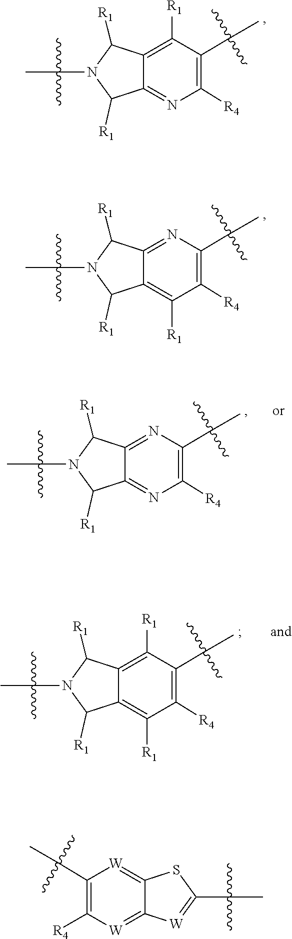

is independently selected from

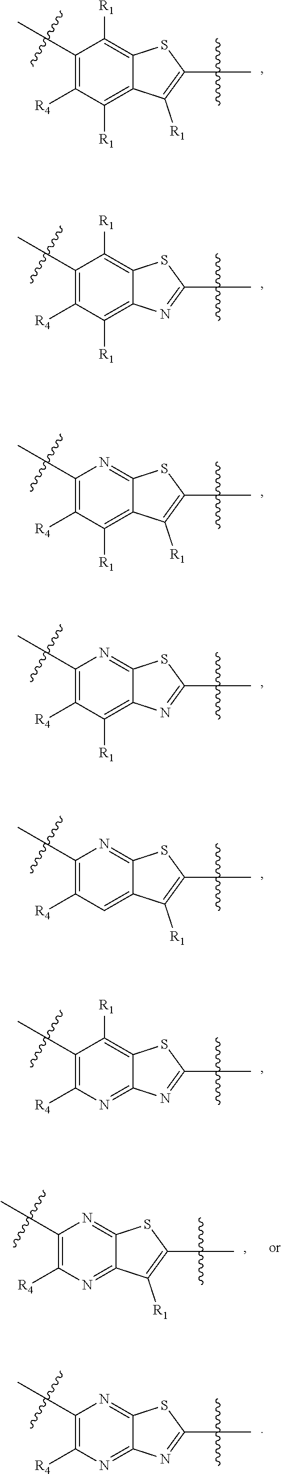

In some embodiments of Formula II,

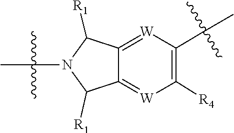

is independently selected from

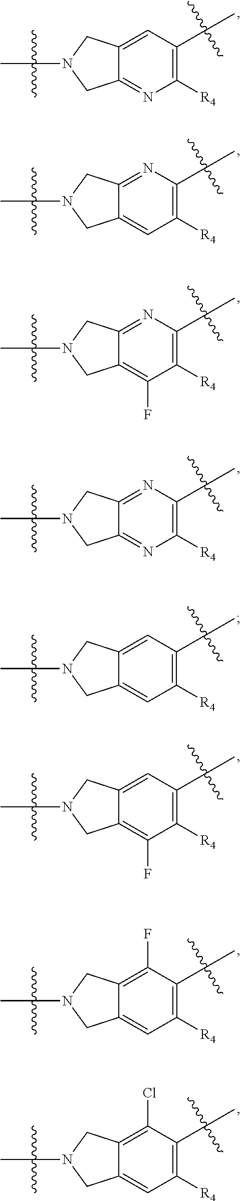

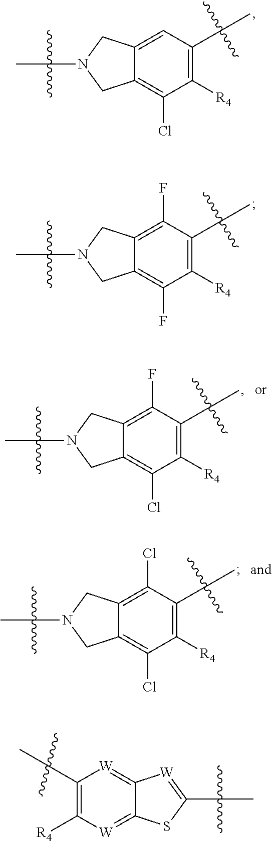

is independently selected from

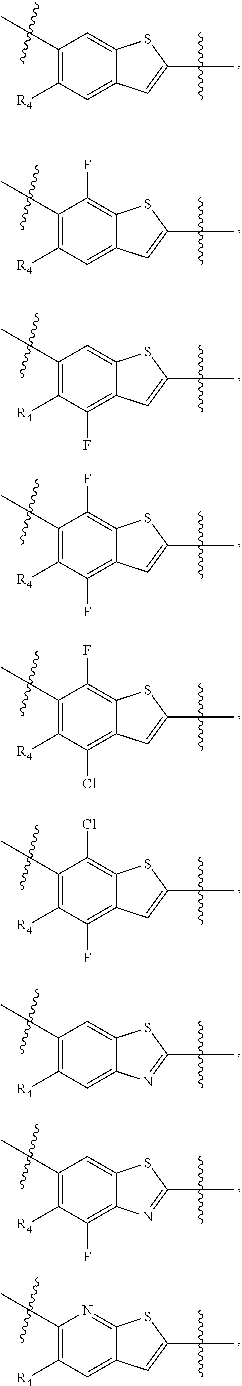

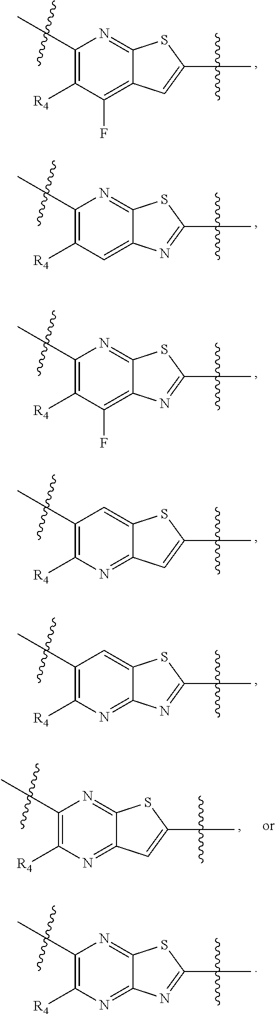

In some embodiments of Formula III,

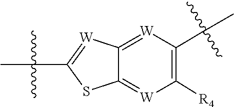

is independently selected from

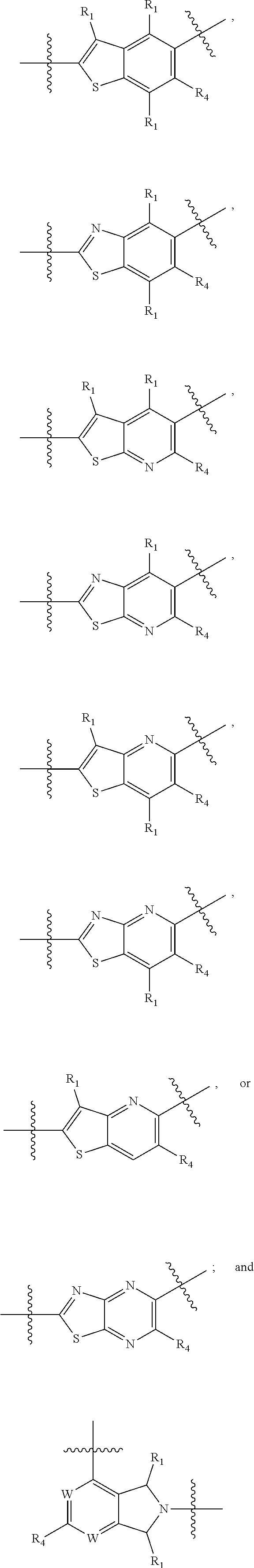

is independently selected from

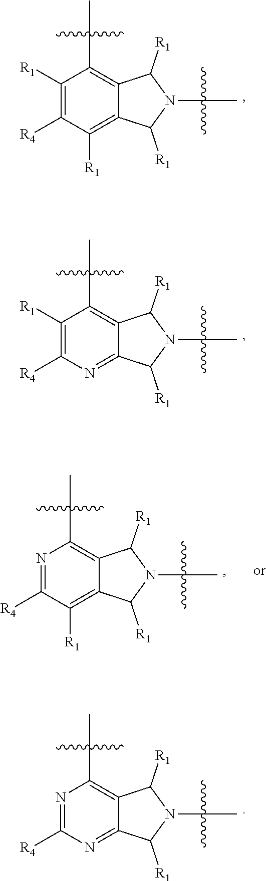

In some embodiments of Formula III,

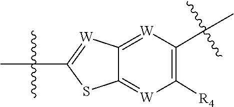

is independently selected from

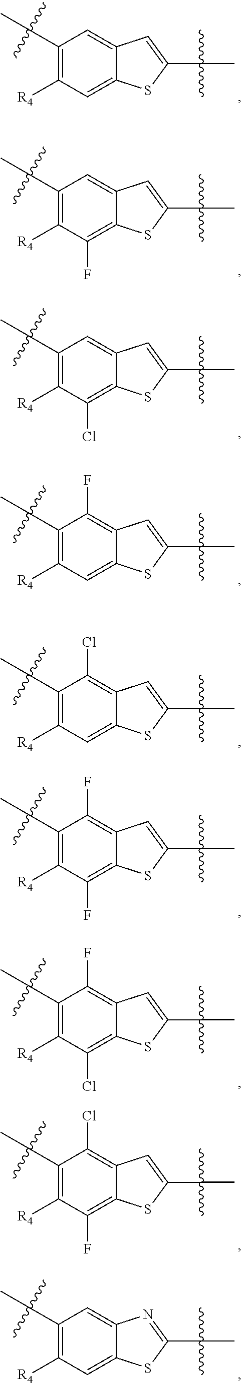

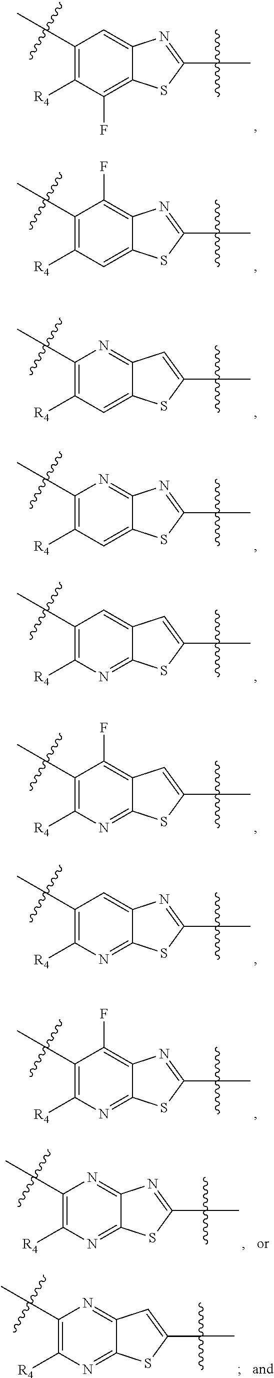

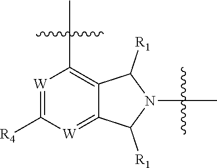

is independently selected from

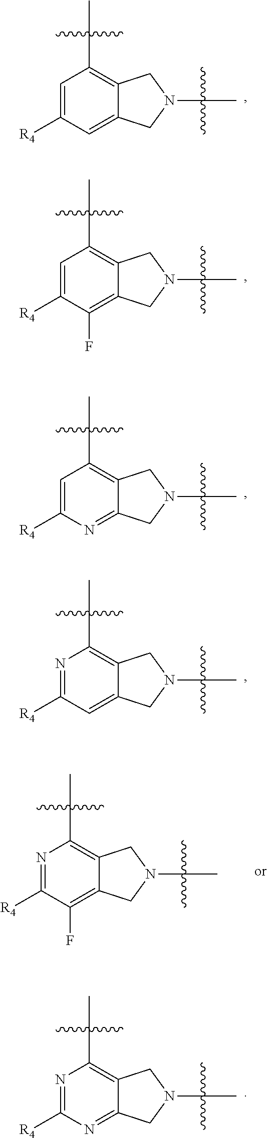

In some embodiments of Formula IV,

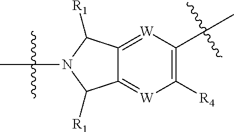

is independently selected from

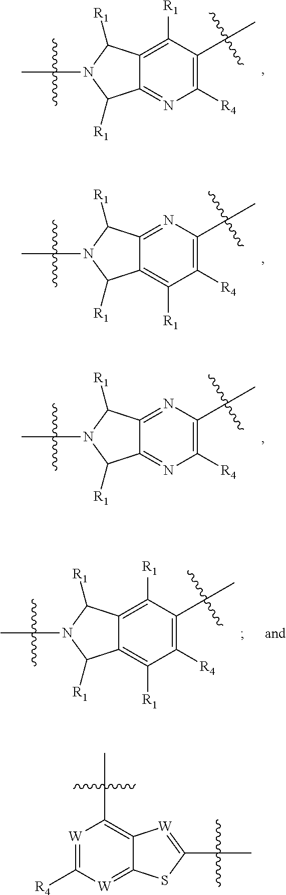

is independently selected from

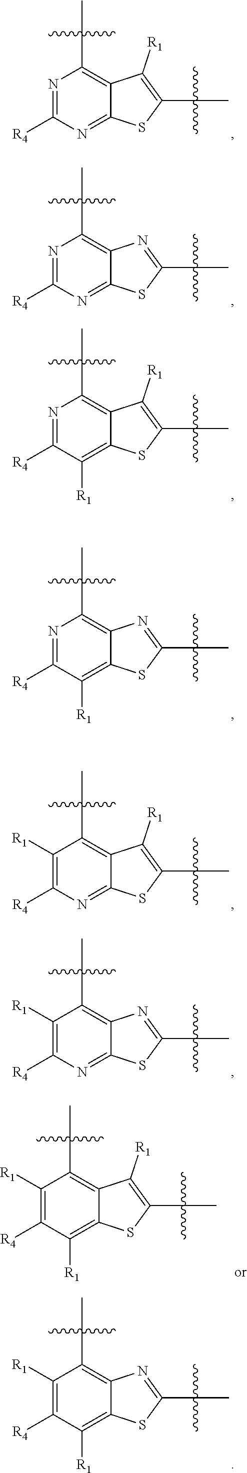

In some embodiments of Formula IV,

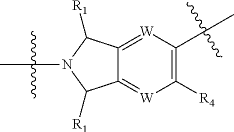

independently selected from

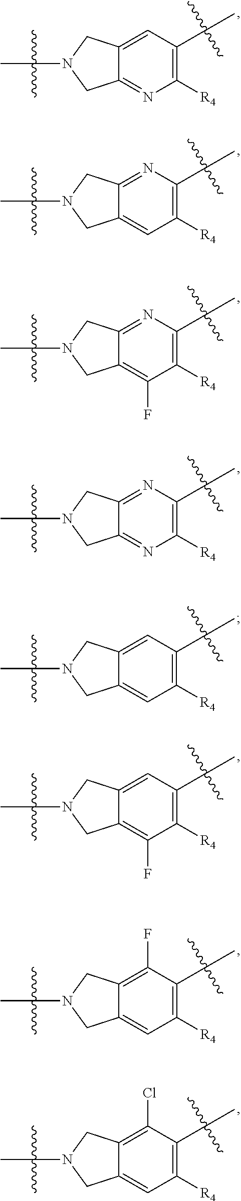

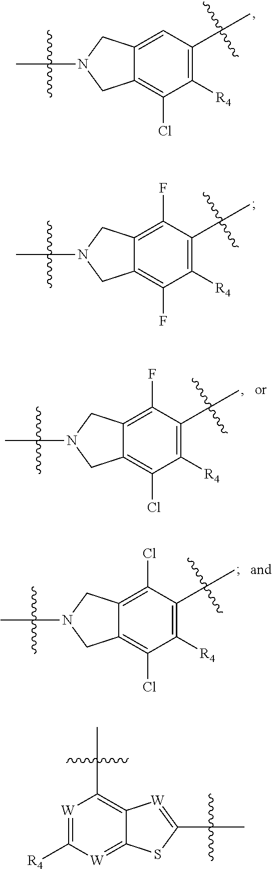

independently selected from

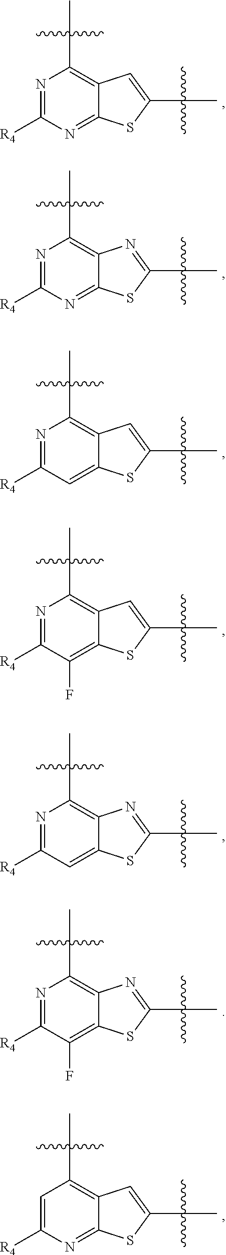

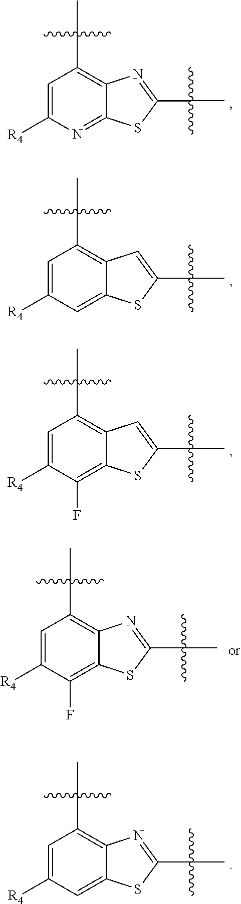

In some embodiments of Formula V,

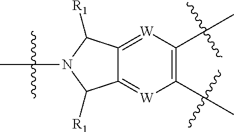

is indendently selected from

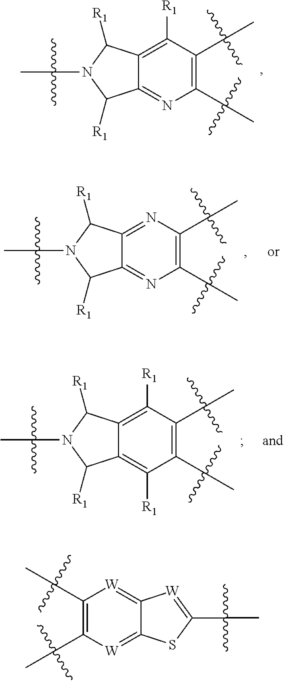

is independently selected from

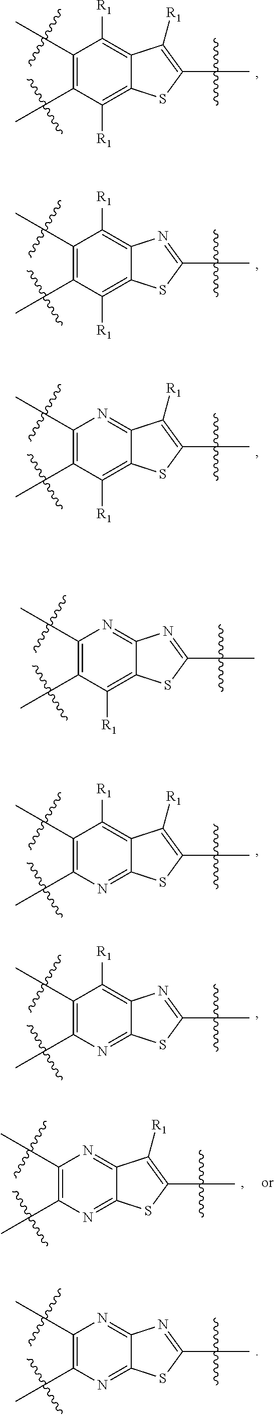

In some embodiments of Formula V,

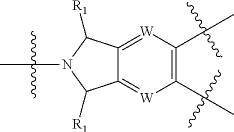

is independently selected from

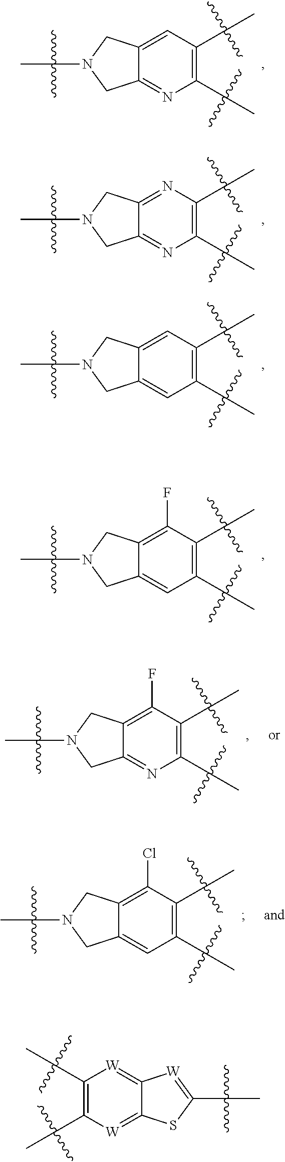

and is independently selected from

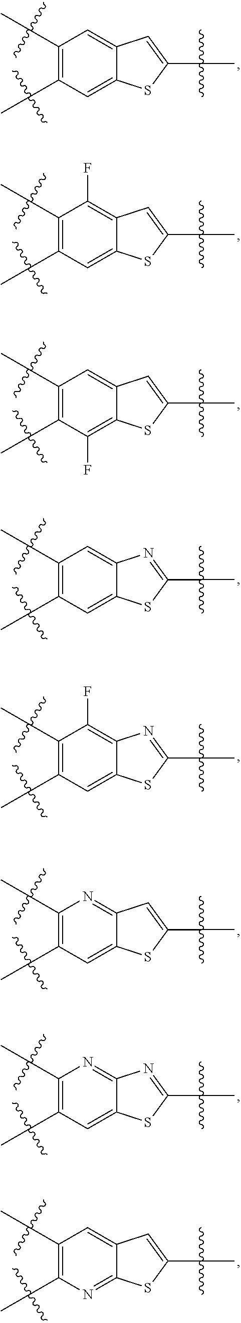

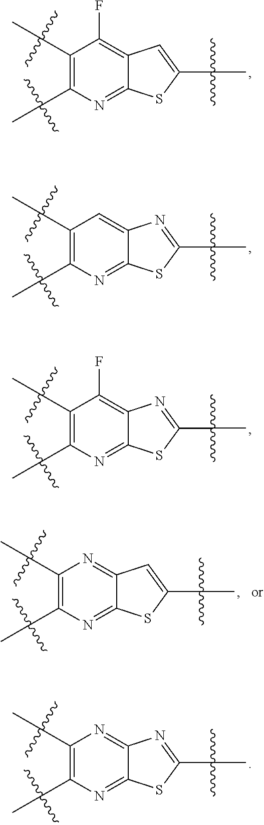 [SMILES: *c1cc2c(F)c(*)c(*)nc2s1 ; *c1nc2cc(*)c(*)nc2s1 ; *c1nc2c(F)c(*)c(*)nc2s1 ; *c1cc2nc(*)c(*)nc2s1 ; *c1nc2nc(*)c(*)nc2s1]

In some embodiments of Formula I, I-1, II, III, IV or V, each W is independently is CR₁.

In some embodiments of Formula I, I-1, II, III, IV or V, each W is independently is CH or CF.

In some embodiments of Formula I, I-1, II, III, IV or V, each W is independently is N.

In some embodiments of Formula I, I-1, II, III, IV or V, each R₁ is independently selected from H, deuterium, halogen, OR⁶, N(R⁶)₂, COOR⁶, or C(O)N(R⁶)₂, CN or C₁-C₆ alkyl; wherein the C₁-C₆ alkyl is optionally substituted with one or more deuterium, halogen, OR⁶, N(R⁶)₂, COOR⁶, or C(O)N(R⁶)₂.

In some embodiments of Formula I, I-1, II, III, IV or V, each R₁ is independently selected from the group consisting of H, deuterium, halogen, C₁-C₃ alkyl, CN and C₁-C₃ haloalkyl.

In some embodiments of Formula I, I-1, II, III, IV or V, wherein each R₁ is independently selected from the group consisting of H, deuterium, halogen, CN and C₁-C₃ alkyl.

In some embodiments of Formula I, I-1, II, III, IV or V, wherein each R₁ is independently selected from the group consisting of H, deuterium, F, Cl, Br, CN and methyl.

In some embodiments, each R₁ independently is hydrogen or halogen.

In some embodiments, each R₁ independently is hydrogen or F In some embodiments, each R₁ independently is hydrogen or CN.

In some embodiments, each R₁ independently is deuterium.

In some embodiments of Formula I, I-1, II, III, IV or V, R₂ and R₃ are independently selected from -Tₐ-C₁-C₆ alkyl-T_{b}-, -Tₐ- N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}- , -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃-C₁₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ- (C₃-C₁₂cycloalkyl)-T_{b}-, -Tₐ- (C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs.

In some embodiments of Formula I, I-1, II, III, IV or V, R₂ and R₃ are independently selected from -Tₐ-C₁-C₆ alkyl-T_{b}-, -Tₐ- N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}-, -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃-C₁₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ- (C₃-C₁₂cycloalkyl)-T_{b}-, -Tₐ- (C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs, wherein in -Tₐ- (C₃-C₁₂ cycloalkyl)-T_{b}- or -Tₐ-(3- to 12-membered heterocycloalkyl)-T_{b}-, the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O— (C₁-C₆ alkyl)-O—; wherein the C₁-C₆ alkyl is optionally substituted with one or more halogen; and
each Rs independently is H, deuterium or C₁-C₆ alkyl optionally substituted with one or more halogen.

In some embodiments of Formula I, I-1, II, III, IV or V, R₂ and R₃ are independently selected from -Tₐ-C₁-C₆ alkyl-T_{b}-, -Tₐ- N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}-, -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ- (C₃-C₁₂cycloalkyl)-T_{b}-, -Tₐ- (C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs.

In some embodiments of Formula I, I-1, II, III, IV or V, R₂ and R₃ are independently selected from -Tₐ-C₁-C₆ alkyl-T_{b}-, -Tₐ- N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}- , -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═S)- T_{b}-, -Tₐ-S(═O)₂-T_{b}-, -Tₐ-S(═O)-T_{b}-, -Tₐ-P(═O)(—ORs)-T_{b}-, -Tₐ- (C₃-C₁₂cycloalkyl)-T_{b}-, -Tₐ- (C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs; and wherein the C₃-C₁₂ cycloalkyl or 3- to 12-membered heterocycloalkyl is attached to Tₐ and T_{b}respectively via two different atoms of the C₃-C₁₂ cycloalkyl or 3- to 12-membered heterocycloalkyl;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O— (C₁-C₆ alkyl)-O—; wherein the C₁-C₆ alkyl is optionally substituted with one or more halogen; and each Rs independently is H, deuterium or C₁-C₆ alkyl optionally substituted with one or more halogen.

In some embodiments of Formula I, I-1, II, III, IV or V, R₂ and R₃ are independently selected from O—(C₁-C₄ alkylene or haloalkylene)-C₂-C₆alkenyl, C₁-C₅ alkylene or haloalkylene, (C₁-C₄ alkylene or haloalkylene)-N(R⁶), and N(R⁶)—(C₁-C₄ alkylene or haloalkylene)- C₂-C₆alkenyl, —C₀₋₆alkyl-NH—C₀₋₆alkyl-, —C₀₋₆alkyl-N(C_{1 6}alkyl)-C₀₋₆alkyl-, —C₀₋₆alkyl-O—C₀-6alkyl-, —C₀₋₆alkyl-PEGₙ-O—C₀₋₆alkyl, —C₀₋₆alkyl-S—S—C₀-6alkyl, —C₀-6alkyl-S—S—S—C₀₋₆alkyl, —C₀₋₆ alkyl-C₂-C₆alkenyl-C₀₋₆ alkyl-, —C₀₋₆ alkyl-C₂-C₆alkynyl- C₀₋₆ alkyl-, —C₀₋₆ alkyl-C(═O)—Co₆ alkyl-, —C₀₋₆ alkyl-C(═CH₂)—C₀₋₆alkyl-, —C₀₋₆ alkyl-C(═O)—C(═O)—C₀₋₆alkyl-, —C₀₋₆ alkyl-C(═S)—C₀₋₆alkyl-, —C₀₋₆ alkyl-S(═O)₂—C₀₋₆alkyl-, —C₀₋₆alkyl-S(═O)—C₀₋₆alkyl-, —C₀₋₆ alkyl-P(═O)(—OH)—C₀₋₆alkyl-, —C₀₋₆alkyl-C₃-C₁₂ cycloalkyl-C₀-6alkyl-, —CO₁₋₆ alkyl-C₆-C₁₂ aryl-C₀₋₆alkyl-, —CO-₆ alkyl-(3- to 12-membered heterocyclyl)-C₀₋₆ alkyl-, —CO-₆ alkyl-(5- to 12-membered heteroaryl)-C₀₋₆ alkyl-, —C₀₋₆alkyl-O-(5- to 12-membered heteroaryl)-O—C₀₋₆alkyl-, —C₀₋₆alkyl-O—C(═O)—NH—C₀₋₆alkyl-, —C₀₋₆alkyl-O—C(═O)—C₀₋₆alkyl-, —C₀₋₆alkyl-NH—C(═O)—C₀₋₆alkyl-, —OC(═O)—O—, —NH—C(═O)—NH—, or —NH—C(═S)—NH—; wherein the C₂-C₆ alkenyl, C₂-C₆alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs.

In some embodiments of Formula I, I-1, II, III, IV or V, R₂ and R₃ are independently selected from -Tₐ-C₂-C₆ alkenyl-T_{b}-, -Tₐ-C(═O)-T_{b}-, -Tₐ-PEGₙ-O-T_{b}, -Tₐ-S—S-T_{b}, -Tₐ-S—S—S-T_{b}, -Tₐ-C(═CH₂)-T_{b}-, or -Tₐ- (C₆-C₁₂ aryl)-T_{b}-, wherein the C₂-C₆alkenyl or C₆-C₁₂ aryl is optionally substituted with one or more deuterium, halo, —ORs, —N(Rs)₂, or —C(═O)ORs;
PEGₙ is (—OCH₂CH₂—)ₙ, n=1-8;
Tₐ and T_{b} each independently are —N(Rs)-, —O—, —(C₁-C₆ alkyl)-O—, or —O—(C₁-C₆ alkyl)-O—; wherein the C₁-C₆alkyl it optionally substituted with one or more halogen; each Rs independently is H, deuterium or C₁-C₆ alkyl optionally substituted with one or more halogen.

In some embodiments of Formula I, I-1, II, III, IV or V, wherein R₂—R₃ is selected from

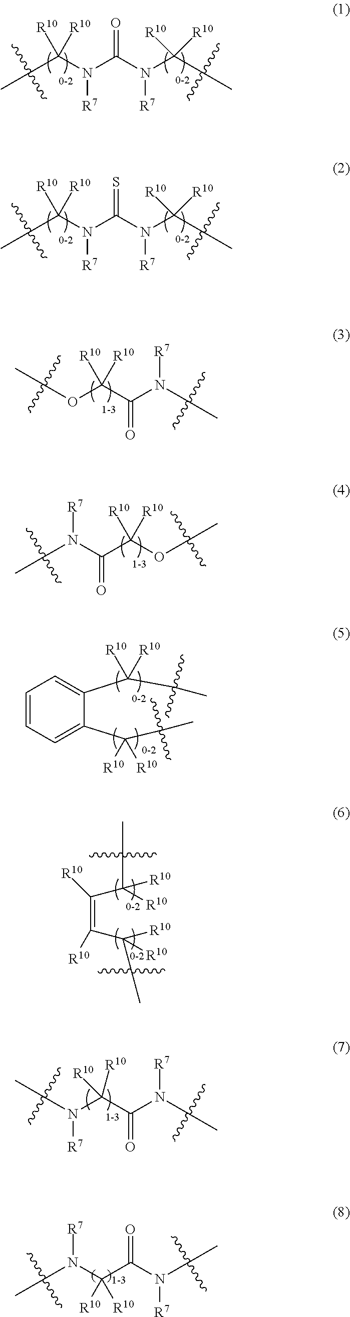

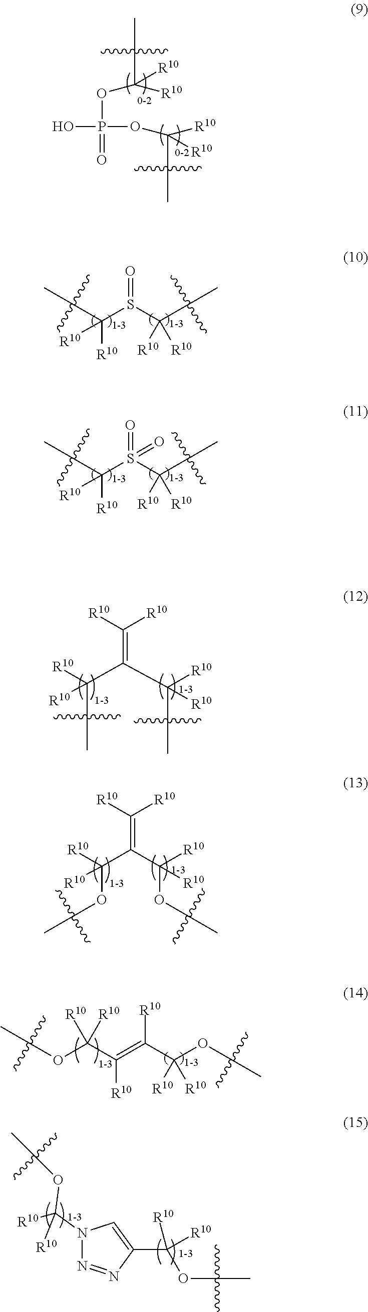

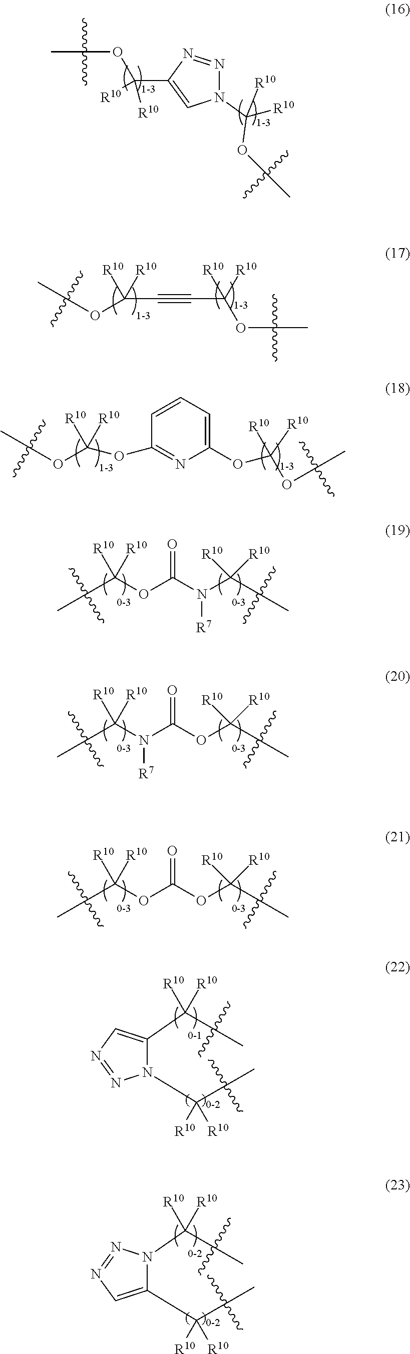

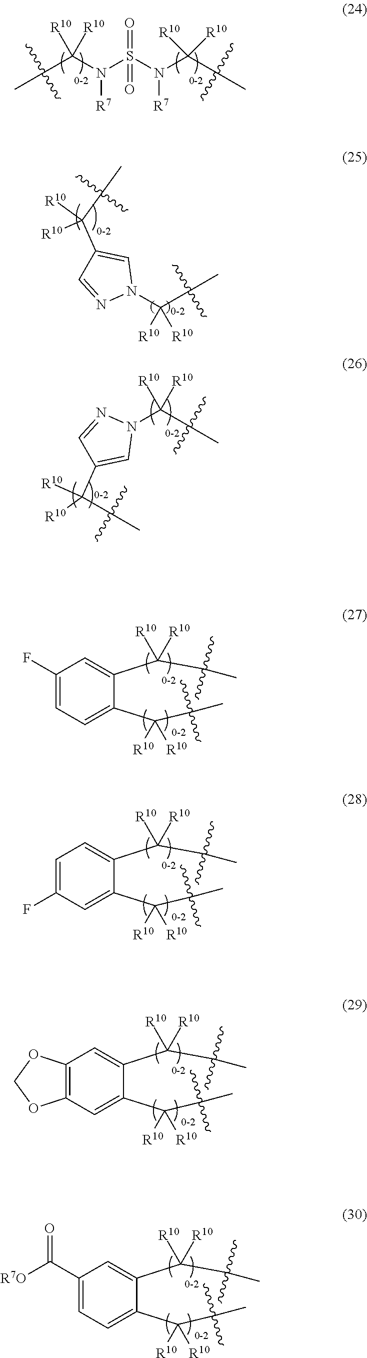

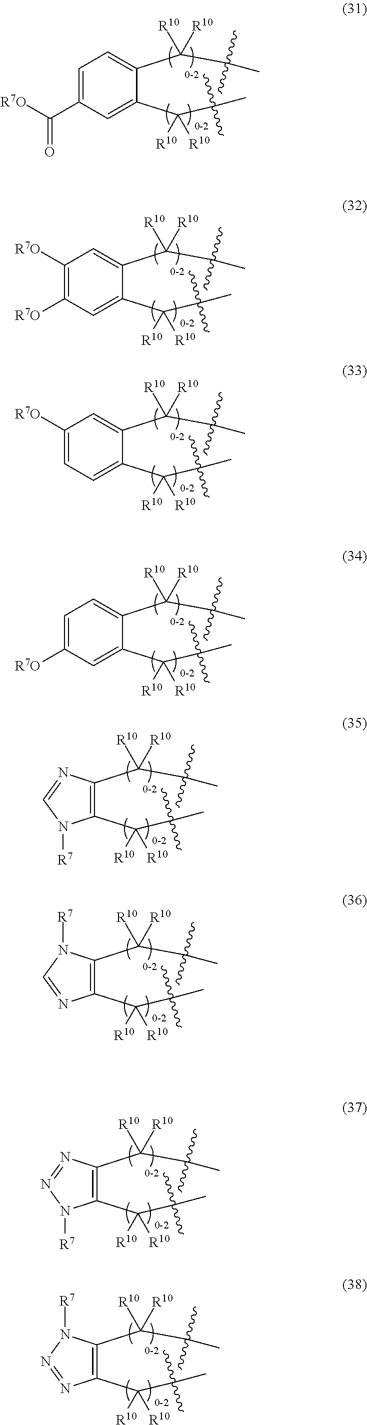

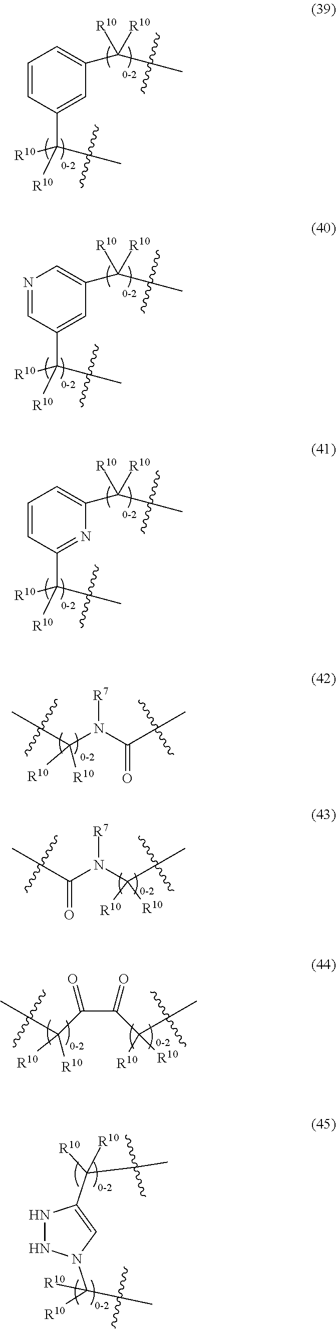

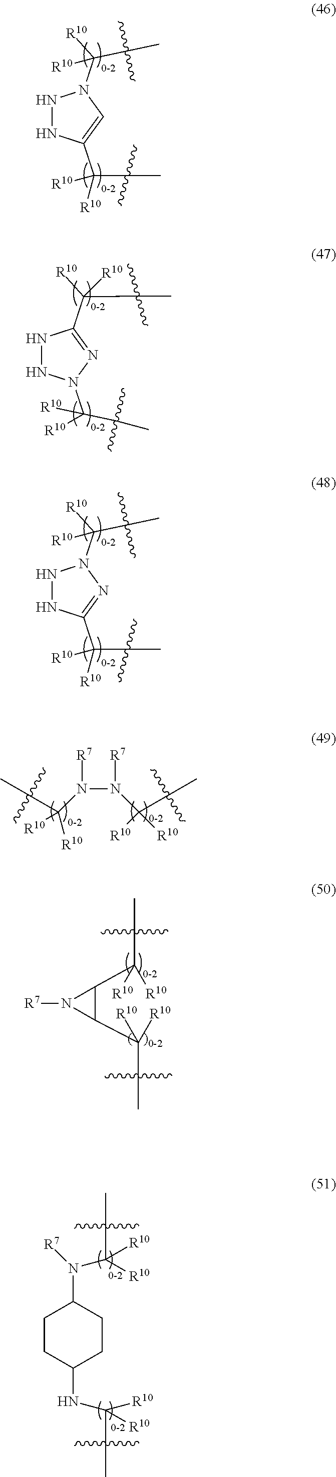

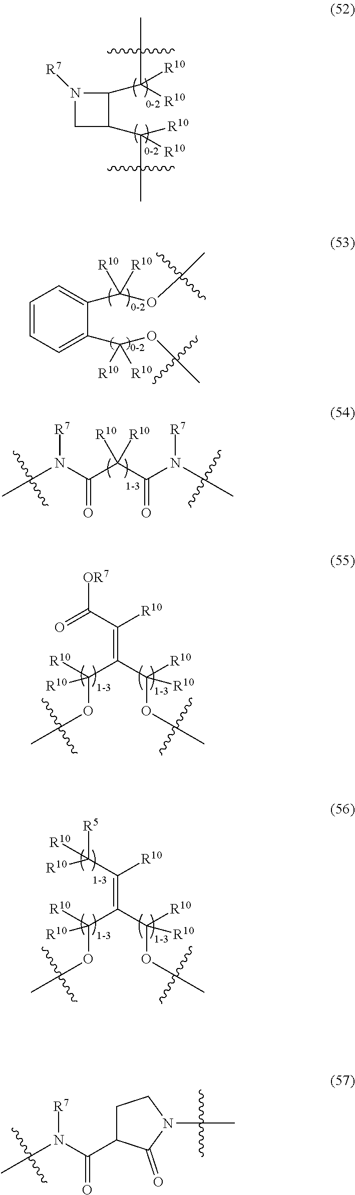

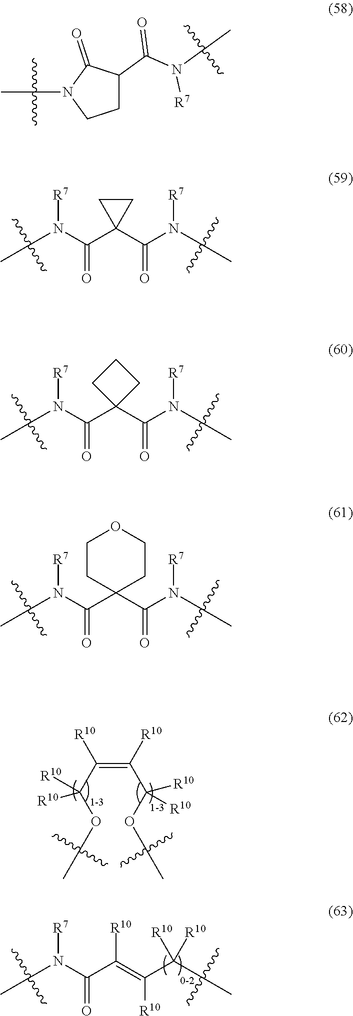

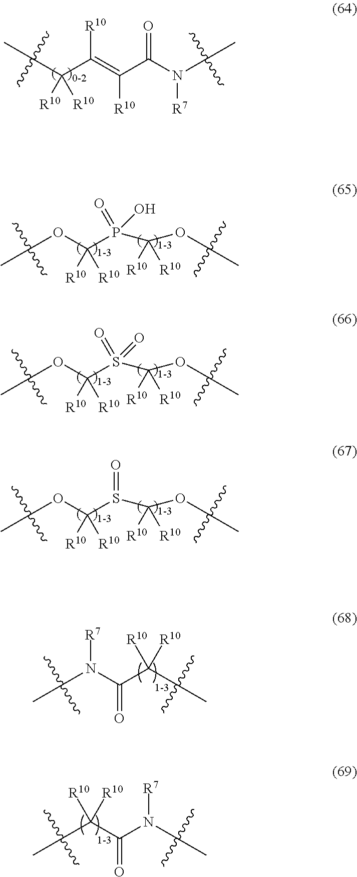

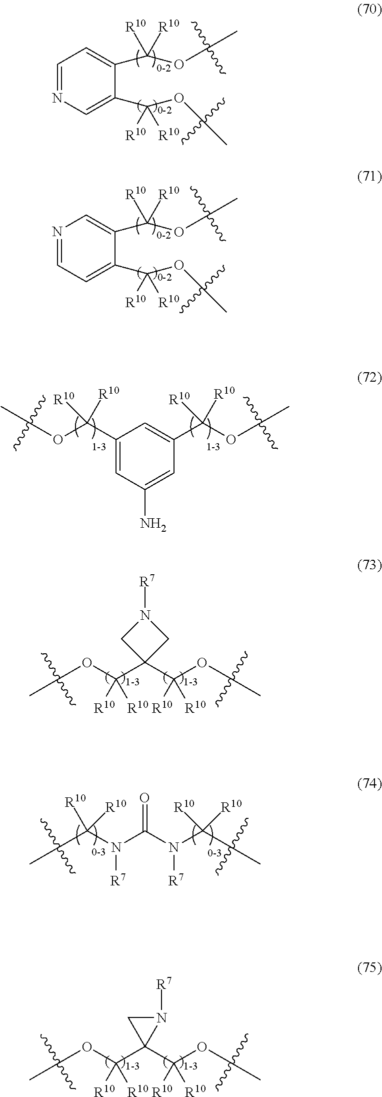

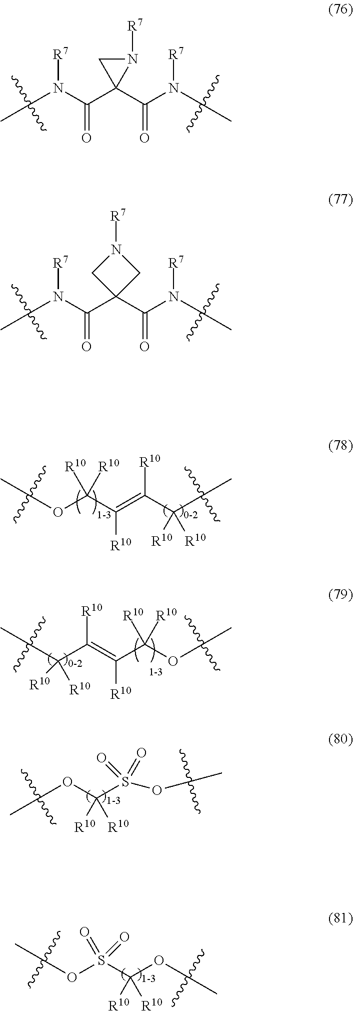

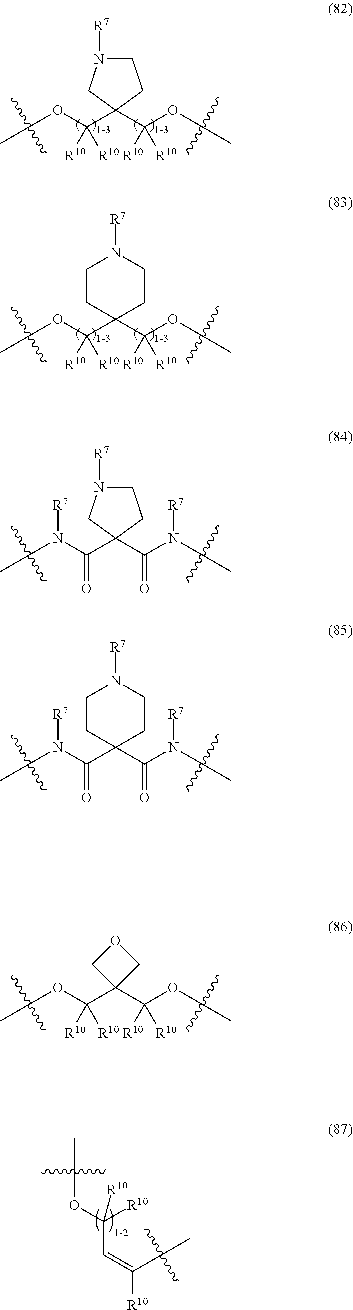

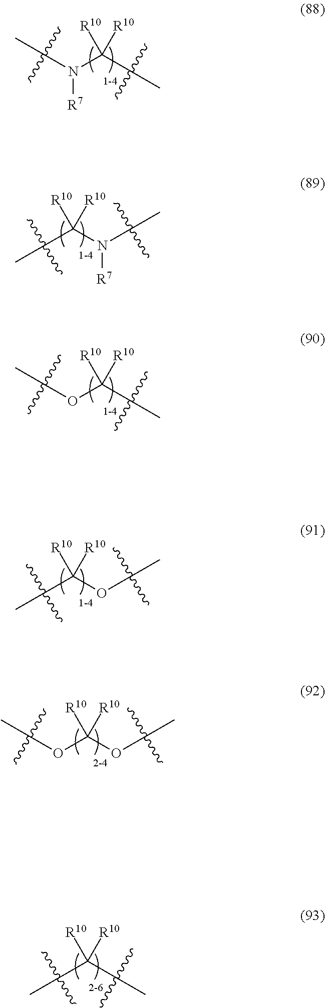

wherein:
each R₅ is independently —OR⁷, NR⁷ or —C(O)OR⁷;
each R⁷ is independently hydrogen, deuterium or C_{1 2} alkyl; and
each R¹⁰ is independently hydrogen, deuterium, C₁₋₂ alkyl or halogen.

In some embodiments of Formula I, I-1, II, III, IV or V, wherein each R⁷ is independently hydrogen, deuterium or methyl.

In some embodiments, each R¹⁰ is independently hydrogen, deuterium, methyl or fluorine.

In some embodiments, one R¹⁰ is hydrogen, and the other R¹⁰ is methyl or fluorine.

In some embodiments, one R¹⁰ is deuterium, and the other R¹⁰ is methyl or fluorine.

In some embodiments of Formula I, I-1, II, III, IV or V, wherein R₂—R₃ is selected from the group consisting of —(CH₂—C₁CH₂)₁-r, —O(CH₂)₁₋₆O—, —OCH₂CH(CH₃)CH₂O—, —OCH(CH₃)CH₂CH(CH₃)O—,

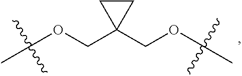 [SMILES: *OCC1(CO*)CC1]

—NH(CH₂)₁₋₇—, —(CH₂)₁₋₆NH(CH₂)₁₋₆—, —(CH₂)₁₋₆N(CH₃)(CH₂)₁₋₆—, —NH(CH₂)₁₋₆O—, —NH—CO—CH₃ CH₃ NH—, —N(CH₃)CO—NH—,

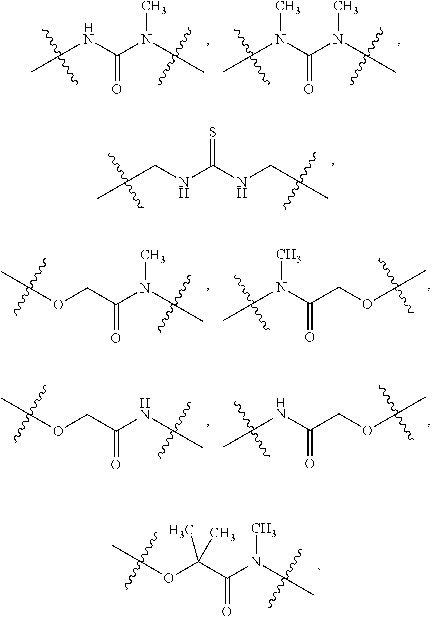

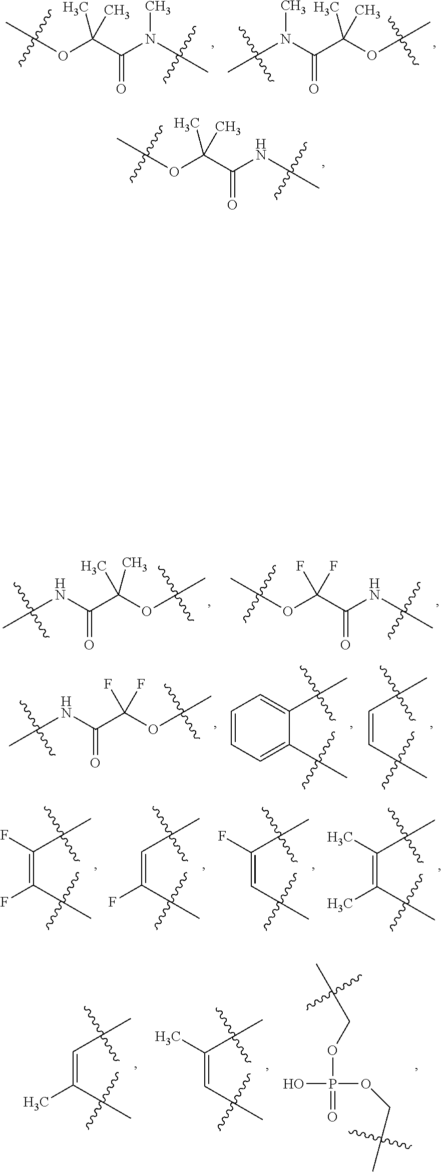

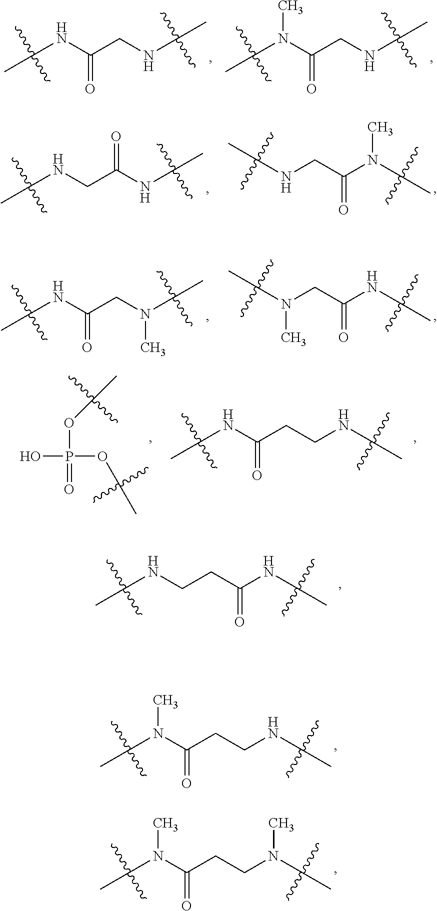

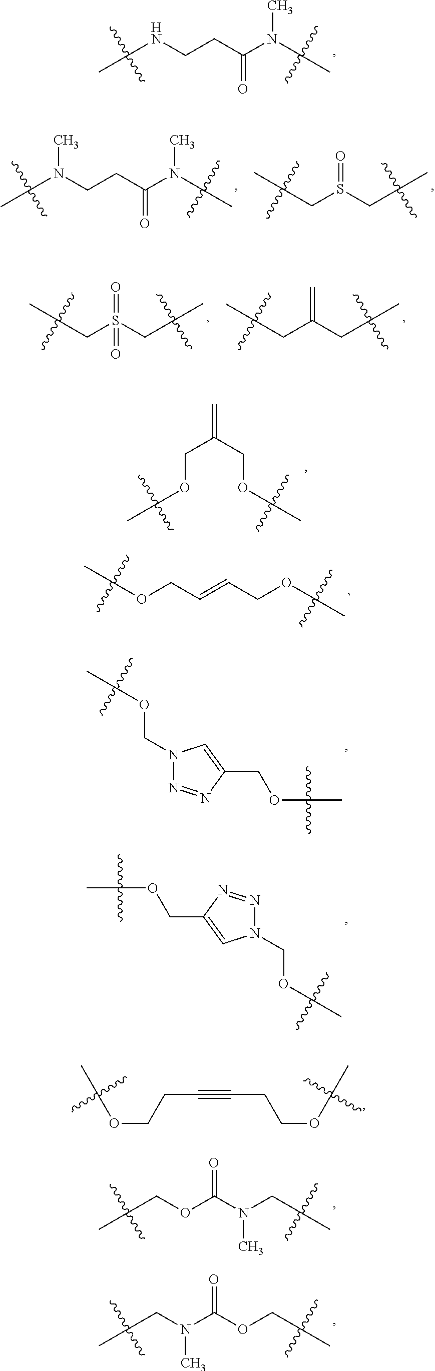

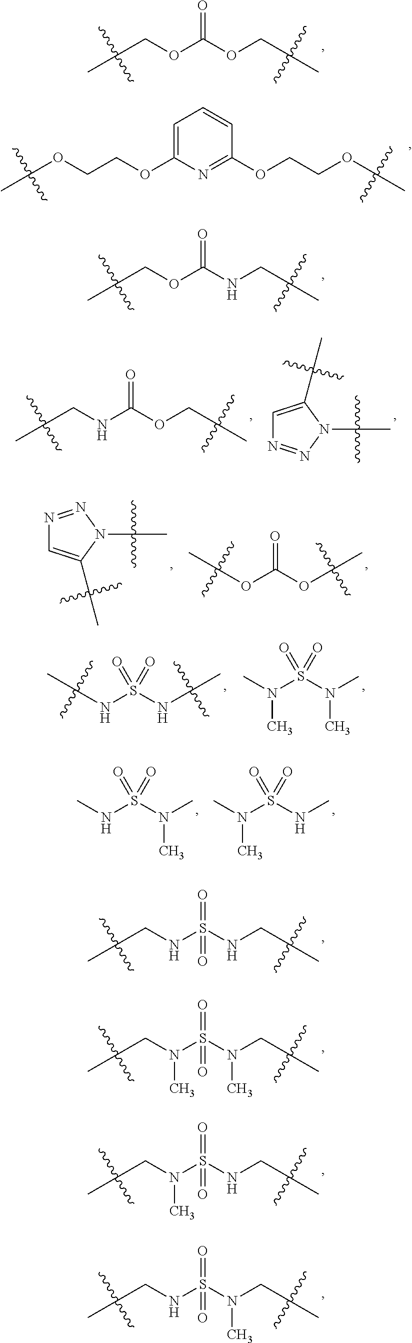

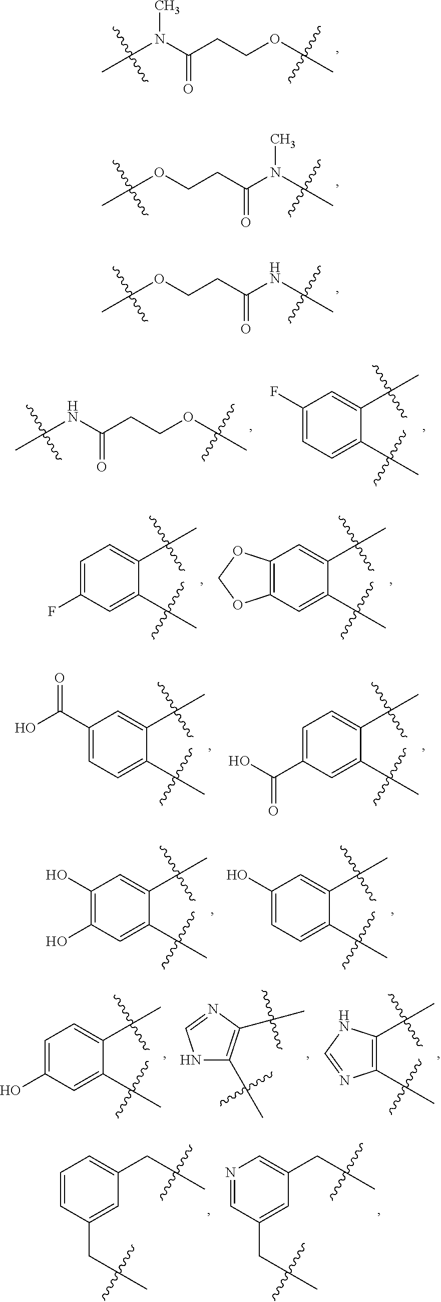

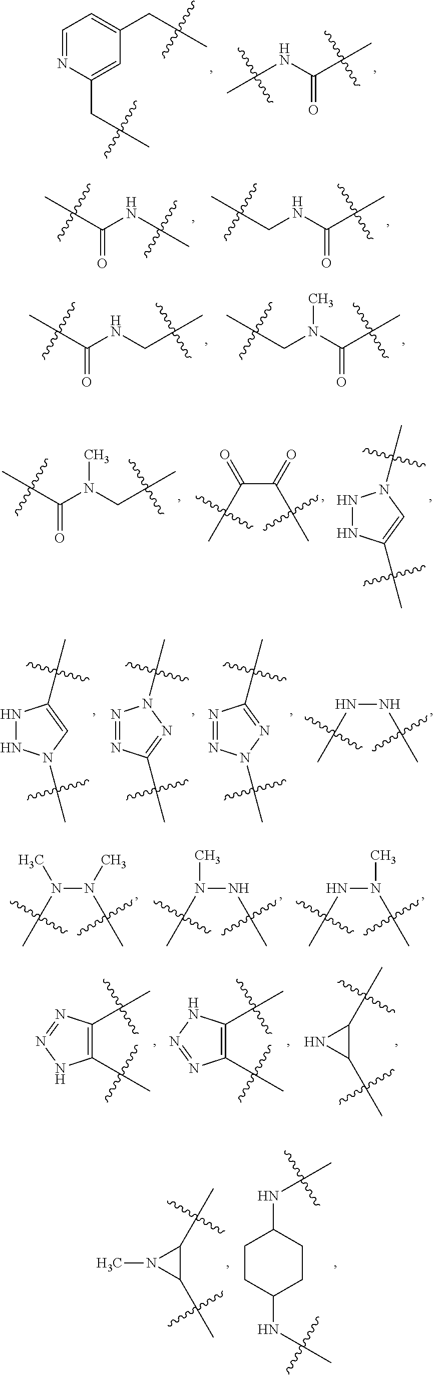

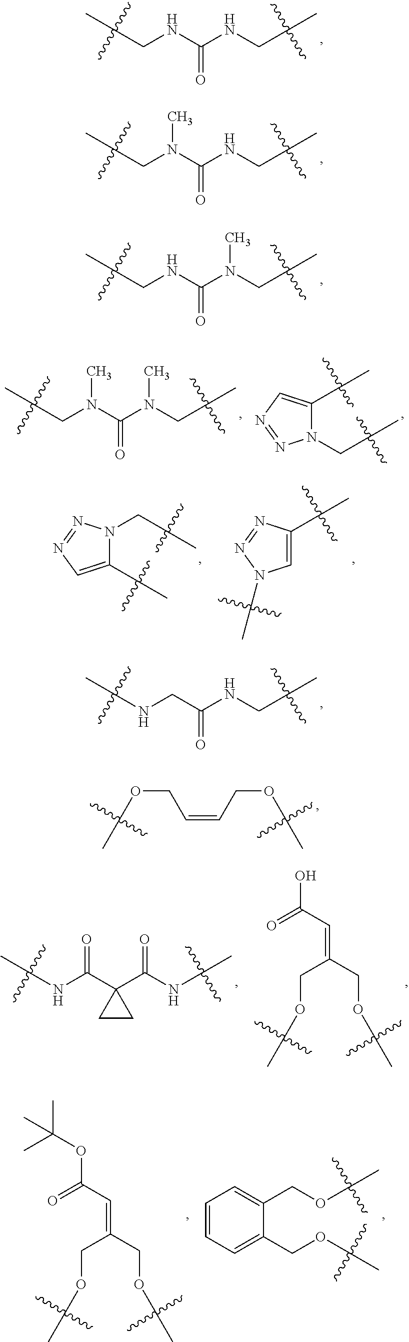

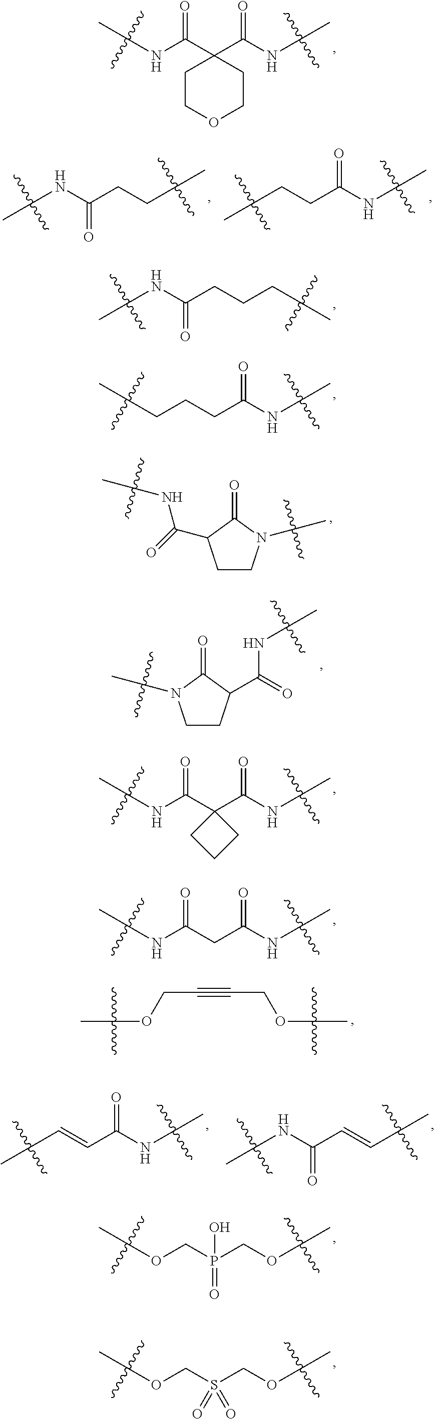

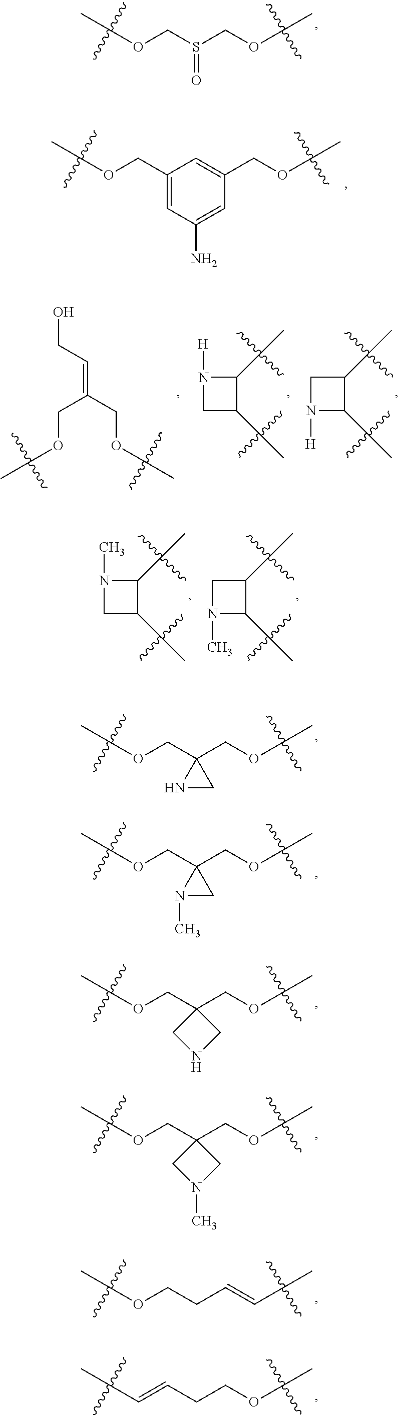

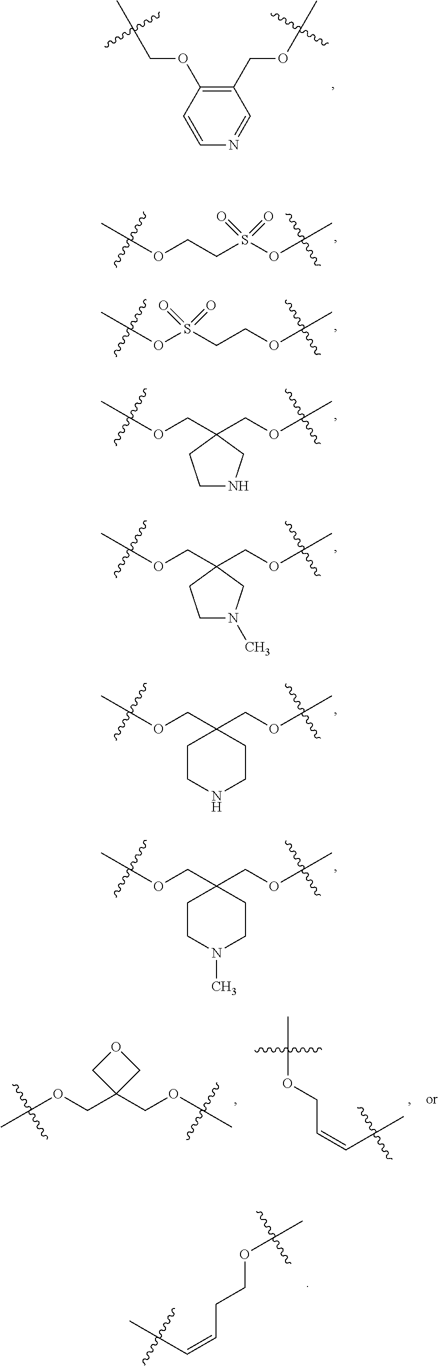

In some embodiments of Formula I, I-1, II, III, IV or V, wherein R₂—R₃ is selected from the group consisting of —(CH₂)₂—, —(CH₂)₃—, —(CH₂)₄—, —(CH₂)₅—, —O(CH₂)₂—, —O(CH₂)₃—, —O(CH₂)₄—, —O CH₂)₂O—, —O(CH₂)₃O—, —O(CH₂)₄O—, —OCH₂CH(CH₃)CH₂O—, —OCH(CH₃)CH₂CH(CH₃)O—, —

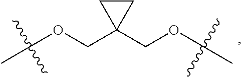 [SMILES: *OCC1(CO*)CC1]

O(CH₂)₄O—, —O(CH₂)₅O—, —NH(CH₂)₂—, —NH(CH₂)₃—, —NH(CH₂)₄—, —(CH₂)₂NH—, —(CH₂)₃NH—, —(CH₂)₄NH—, —CH₂NHCH₂—, —CH₂N(CH₃)CH₂—, —NH(CH₂)₃O—, —NH—CO—NH—,

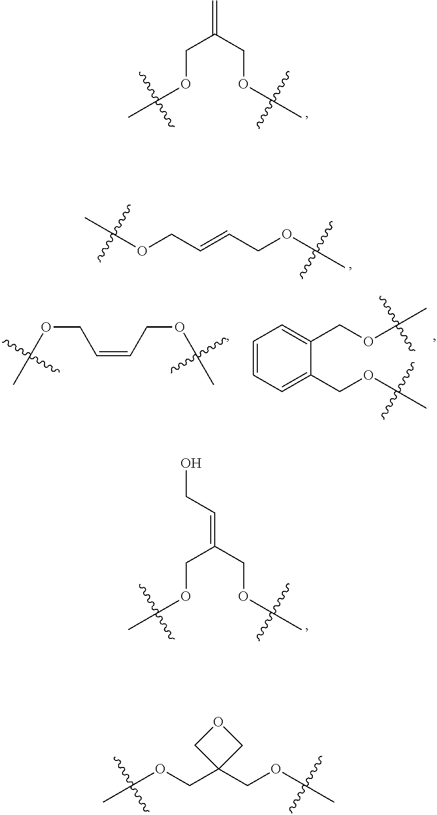

or —N(CH₃)CONH—.

In some embodiments of Formula I, I-1, II, III or IV, each R₄ is independently selected from the group consisting of H, deuterium, F, Cl, Br, I, CN, OR⁶, N(R⁶)₂, COOR⁶, C(O)N(R⁶)₂, SO₂R⁶, C₁-C₆ alkyl, C₁-C₆ haloalkyl, C₁-C₆ alkyl substituted by OR⁶, C₂-C₆ alkenyl, C₂-C₆ haloalkenyl, C₂-C₆ alkenyl substituted by OR⁶, C₂-C₆ alkynyl, C₂-C₆ haloalkynyl, C₂-C₆ alkynyl substituted by OR⁶, C₃-C₆ cycloalkyl, and a 3- to 6-membered heterocyclic ring including 1 to 2 ring members selected from the group consisting of 0, S, and N(R⁶.

In some embodiments of Formula I, I-1, II, III or IV, each R₄ is independently selected from the group consisting of H, deuterium, F, Cl, Br, I, OH, C₁-C₃ alkyl, C₁-C₃ haloalkyl, OC₁-C₃ alkyl, OC₁-C₃ haloalkyl, C₂-C₃ alkenyl, C₂-C₃alkynyl, and N(R⁶)₂.

In some embodiments of Formula I, I-1, II, III or IV, each R₄ independently is selected from the group consisting of H, deuterium, Br, C₁, OH, CH₃, CH₂CH₃, CH═CH₂, C≡CH, OCH₃, OCFH₂, OCF₂H, OCF₃, and N(R⁶)₂.

In some embodiments of Formula I, I-1, II, III or IV, each R₄ independently is selected from the group consisting of H, deuterium, Br, OH, CH₃, CH₂CH₃, CH═CH₂, C≡CH, OCH₃, NH₂ and NHCH₃.

In some embodiments of Formula I, I-1, II, III, IV or V, each R⁶ is independently selected from the group consisting of —H, deuterium, —F, —Cl, —Br, —I, —NH₂, —CN, —OH, —N₃, —NO₂, carboxyl, C₁-C₃alkyl, C₁-C₃alkoxy, C₂ C₄alkenyl, C₂-C₄alkynyl, 6-membered aryl, 7-membered aryl, 8-membered aryl, 5-membered heteroaryl, 6-membered heteroaryl, 7-membered heteroaryl, 8-membered heteroaryl, 5-membered heterocyclic ring, 6-membered heterocyclic ring, 7-membered heterocyclic ring, 8-membered heterocyclic ring, 5-membered carbocyclic ring, 6-membered carbocyclic ring, 7-membered carbocyclic ring, or 8-membered carbocyclic ring; and each of which is independently optionally substituted with deuterium, —F, —Cl, —Br, —I, —NH₂, —CN, —OH, —NO₂, carbonyl, ═O, oxo, carboxyl, C₁-C₃alkoxy, or C₁-C₃alkyl; and each of the heteroaryl and heterocyclic ring contains 1 or 2 heteroatoms selected from N, O or S.

In some embodiments of Formula I, I-1, II, III, IV or V, each R⁶ is independently selected from the group consisting of —H, deuterium, —F, —Cl, —Br, —I, —NH₂, —CN, —OH, —N₃, —NO₂, carboxyl, methyl, ethyl, propyl, isopropyl, methoxy, ethoxy, propoxy, isopropoxy, ethylene, 6-membered aryl, 5-membered heteroaryl, 6-membered heteroaryl, 5-membered heterocyclic ring, 6-membered heterocyclic ring, 5-membered carbocyclic ring, or 6-membered carbocyclic ring; and each of which is independently optionally substituted with deuterium, —F, —Cl, —Br, —I, —NH₂, —CN, —OH, —NO₂, carbonyl, ═O, oxo, carboxyl, methyl, ethyl, propyl, isopropyl, methoxy, ethoxy, propoxy or isopropoxy; and each of the heteroaryl and heterocyclic ring contains 1 or 2 heteroatoms selected from N, O or S.

In some embodiments of Formula I, I-1, II, III, IV or V, each R⁶ is independently selected from the group consisting of H, deuterium, —F, —Cl, —Br, —I, —NH₂, —CN, —OH, methyl, ethyl, propyl, isopropyl, methoxy, ethoxy, propoxy, isopropoxy, CH2F, —CHF2,

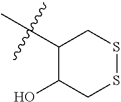 [SMILES: *C1CSSCC1O]

### and —CF3.

In some embodiments of Formula I, I-1, II, III, IV or V, each X₁ is independently selected from the group consisting of C═O, —CH₂—, —CHF—, and —CF₂—.

In some embodiments of Formula I, I-1, II, III, IV or V, each X₁ is selected from the group consisting of C═O and —CH₂—.

In some embodiments of Formula I, I-1, II, III, IV or V, each X₁ is C═O.

In some embodiments of Formula I, I-1, II, III, IV or V, each X₂ is independently selected from (CH₂)₍₁₋₃₎, wherein each R⁸ is independently selected from the group consisting of H, deuterium, halogen, C₁-C₆ alkyl, CN, OR⁶, N(R⁶)₂, C₁-C₆ haloalkyl, C₃-C₆ cycloalkyl, C₁-C₆ alkyl substituted by OR⁶, and C₁-C₆ alkyl substituted by N(R⁶)₂; optionally 2 R⁸ on different carbon atoms may be taken together, along with the atoms to which they are attached, to form a 3- to 6-membered fused ring; and optionally 2 R⁸ on a single carbon atom may be taken together, along with the atom to which they are attached, to form a 3- to 6-membered spirocycle.

In some embodiments, each X₂ independently is —(C(R⁸)₂)₁₋₃—, wherein each R⁸ independently is hydrogen, deuterium, halogen, C₁-C₆ alkyl, CN, OR⁶, N(R⁶)₂, or C₃-C₆ cycloalkyl; wherein the C₁-C₆ alkyl is optionally substituted with one or more halogen, OR⁶, or N(R⁶)₂.

In some embodiments, each X₂ independently is —(C(R⁸)₂)₁₋₃—, wherein at least two R⁸, together with the one or more atoms to which they are attached, form C₃-C₆ cycloalkyl or 3- to 6-membered heterocycloalkyl.

In some embodiments, each X₂ independently is —C(R⁸)₁₋₃—.

In some embodiments, each X₂ independently is —(CH₂)₁₋₃—.

In some embodiments, each X₂ independently is —C(R⁸)₂—.

In some embodiments, each X₂ independently is —CH₂—.

In some embodiments, each X₂ independently is —C(R⁸)₂C(R⁸)₂—.

In some embodiments, each X₂ independently is —CH₂CH₂—.

In some embodiments, each X₂ independently is —C(R⁸)₂C(R⁸)₂C(R⁸)₂—.

In some embodiments, each X₂ independently is —CH₂CH₂CH₂—.

In some embodiments of Formula I, I-1, II, III, IV or V, each X₂ is CH₂CHR⁸, where R⁸ is selected from the group consisting of H, deuterium, C₁-C₃ alkyl, C₁-C₃ alkyl substituted by OH, C₁-C₃ alkyl substituted by OC1-C₃ alkyl, and C₃-C₆ cycloalkyl.

In some embodiments of Formula I, I-1, II, III, IV or V, each X₂ is CH₂CHR⁸, wherein R⁸ is selected from the group consisting of H, deuterium, CH₃, CH₂OH, CH₂CH₃, CH₂CH₂CH₃, CH(CH₃)₂, CH₂OCH₃, and cyclopropyl.

In some embodiments of Formula I, I-1, II, III, IV or V, each X₂ is CHR⁸CHR⁸, where each R⁸ is independently selected from the group consisting of H, deuterium, C₁-C₃ alkyl, C₁-C₃ alkyl substituted by OH, C₁-C₃ alkyl substituted by OC₁-C₃ alkyl, and C₃-C₆ cycloalkyl, and optionally the 2 R⁸ on different carbon atoms are taken together, along with the atoms to which they are attached, to form a 3- to 6-membered fused ring.

In some embodiments of Formula I, I-1, II, III, IV or V, each X₂ is CHR⁸CHR⁸, where each R⁸ is independently selected from the group consisting of H, deuterium and C₁-C₃ alkyl, and optionally the 2 R⁸ on different carbon atoms are taken together, along with the atoms to which they are attached, to form a 3- to 6-membered fused ring.

In some embodiments of Formula I, I-1, II, III, IV or V, each X₂ is CH₂C(R⁸)₂, where each R⁸ is independently selected from the group consisting of H, deuterium, C₁-C₃alkyl, C₁-C₃ alkyl substituted by OH, C₁-C₃ alkyl substituted by OC₁-C₃ alkyl, and C₃-C₆ cycloalkyl, and optionally the 2 R⁸ on a single carbon atom are taken together, along with the atoms to which they are attached, to form a 3- to 6-membered spirocycle.

In some embodiments of Formula I, I-1, II, III, IV or V, each X₂ is CH₂C(R⁸)₂, where each R⁸ is independently selected from the group consisting of H, deuterium and C₁-C₃ alkyl, and optionally the 2 R⁸ on a single carbon atom are taken together, along with the atoms to which they are attached, to form a 3- to 6-membered spirocycle.

In some embodiments of Formula I, I-1, II, III, IV or V, each X₃ is independently selected from the group consisting of COOR⁶, C(O)SR⁶, C(S)OR⁶, SO₂R⁶, C(O)N(R⁹)₂,

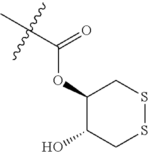 [SMILES: *C(=O)O[C@H]1CSSC[C@@H]1O]

### and CN.

In some embodiments of Formula I, I-1, II, III, IV or V, each X₃ is independently selected from the group consisting of COOR⁶, SO₂R⁶, C(O)N(R⁹)₂,

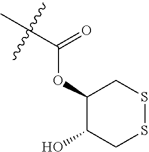 [SMILES: *C(=O)O[C@H]1CSSC[C@@H]1O]

### and CN.

In some embodiments of Formula I, I-1, II, III, IV or V, each X₃ is independently selected from the group consisting of COOR⁶, C(O)N(R⁹)₂,

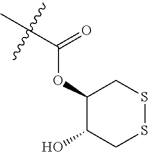 [SMILES: *C(=O)O[C@H]1CSSC[C@@H]1O]

### and CN.

In some embodiments of Formula I, I-1, II, III, IV or V, each X₃ is independently selected from the group consisting of COOH, COOCH₃, CONH₂, CONH—SO₂—N(CH₃)₂, CONH—SO₂—CH₃,

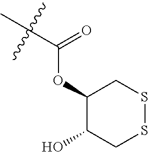 [SMILES: *C(=O)O[C@H]1CSSC[C@@H]1O]

### and CN.

In some embodiments of Formula I, I-1, II, III, IV or V, each R⁹ is independently selected from the group consisting of H, deuterium, COOR⁶, and SO₂R⁶.

In some embodiments of Formula I, I-1, II, III, IV or V, each R⁹ is independently H or deuterium, preferably H.

In some embodiments, the compound is of Formula Ia, IIa, IIIa, IVa or Va:

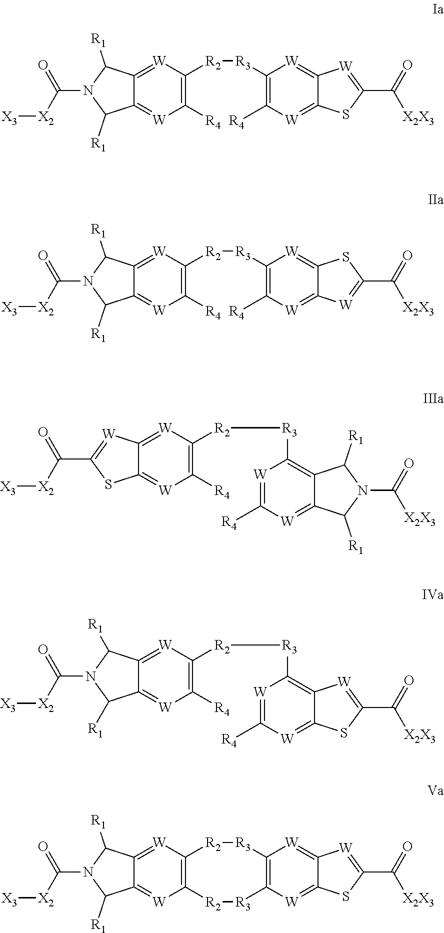

or a pharmaceutically acceptable salt thereof.

In some embodiments, the compound is of Formula lb, IIb, IIIb, or IVb:

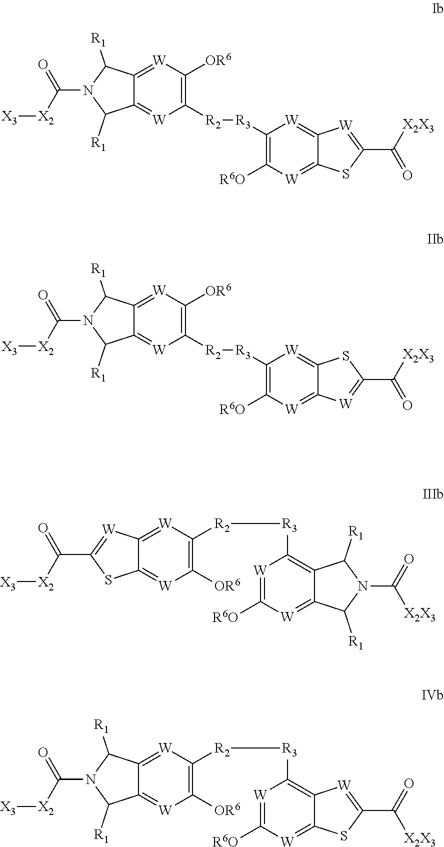

or a pharmaceutically acceptable salt thereof.

In some embodiments, the compound is of Formula Ic, IIc, IIIc, IVc or Vc:

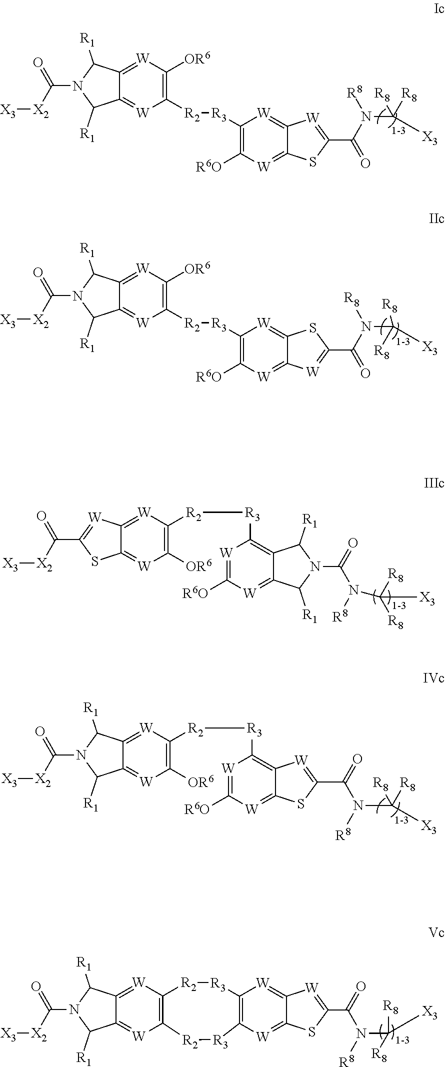

or a pharmaceutically acceptable salt thereof.

In some embodiments of the preceding Formulae defined herein, each heterocyclic ring group and each carbocyclic ring group includes single ring, spiral ring, bridge ring, fused ring and various combinations of spiral ring, bridge ring and/or fused ring.

In some embodiments of the preceding Formulae defined herein, the compound is selected from the compounds described in Table 2 and pharmaceutically acceptable salts thereof.

TABLE 2

| | |
|---|---|
| 1. | 4-(5-(3-((2-(3-carboxypropanoyl)-6-methoxybenzo[b]thiophen- |
| | 5-yl)oxy)propoxy)-6-methoxyisoindolin-2-yl)-4-oxobutanoic |
| | acid |
| 2. | 4-(5-(3-((2-(3-carboxypropanoyl)-4-chloro-6- |
| | methoxyisoindolin-5-yl)oxy)propoxy)-4-fluoro-6- |
| | methoxybenzo[b]thiophen-2-yl)-4-oxobutanoic acid |
| 3. | 4-(5-(3-((2-(3-carboxypropanoyl)-4-chloro-6-methoxybenzo |
| | [b]thiophen-5-yl)oxy)propoxy)-4-fluoro-6-methoxyisoindolin- |
| | 2-yl)-4-oxobutanoic acid |
| 4. | 4-(5-(3-((2-(3-carboxypropanoyl)-4-fluoro-6-methoxybenzo |
| | [b]thiophen-5-yl)oxy)propoxy)-4-fluoro-6-methoxyisoindolin- |
| | 2-yl)-4-oxobutanoic acid |
| 5. | sodium (S)-4-(5-(3-((2-((S)-3-carboxylatobutanoyl)-6- |
| | methoxybenzo[b]thiophen-5-yl)oxy)propoxy)-6- |
| | methoxyisoindolin-2-yl)-2-methyl-4-oxobutanoate |
| 6. | sodium (S)-4-(5-(3-((2-((S)-3-carboxylatobutanoyl)-4-fluoro- |
| | 6-methoxybenzo[b]thiophen-5-yl)oxy)propoxy)-6- |
| | methoxyisoindolin-2-yl)-2-methyl-4-oxobutanoate |
| 7. | sodium 4-(5-(3-((2-(3-carboxylatopropanoyl)-6- |
| | hydroxybenzo[b]thiophen-5-yl)oxy)propoxy)-6- |
| | methoxyisoindolin-2-yl)-4-oxobutanoate |
| 8. | sodium 4-(5-(3-((2-(3-carboxylatopropanoyl)-6- |
| | methoxyisoindolin-5-yl)oxy)propoxy)-6-methoxythieno |
| | [3,2-b]pyridin-2-yl)-4-oxobutanoate |
| 9. | 4-(5-(3-((6-bromo-2-(3-carboxypropanoyl)benzo[b]thiophen- |
| | 5-yl)oxy)propoxy)-6-methoxyisoindolin-2-yl)-4-oxobutanoic |
| | acid |
| 10. | 4-(5-(3-((2-(3-carboxypropanoyl)-4-fluoro-6- |
| | methoxyisoindolin-5-yl)oxy)propoxy)-6- |
| | methoxybenzo[b]thiophen-2-yl)-4-oxobutanoic acid |
| 11. | 4-(5-(3-((6-(3-carboxypropanoyl)-3-methoxy-6,7-dihydro-5H- |
| | pyrrolo[3,4-b]pyridin-2-yl)oxy)propoxy)-6-methoxythieno |
| | [3,2-b]pyridin-2-yl)-4-oxobutanoic acid |
| 12. | 4-(5-(3-((2-(4-ethoxy-4-oxobutanoyl)-6-methoxybenzo[b] |
| | thiophen-5-yl)oxy)propoxy)-6-methoxyisoindolin-2-yl)-4- |
| | oxobutanoic acid |
| 13. | sodium 4-(5-(3-((2-(3-carboxylatopropanoyl)-5-methoxythieno |
| | [2,3-b]pyridin-6-yl)oxy)propoxy)-6-methoxyisoindolin-2-yl)- |
| | 4-oxobutanoate |
| 14. | 4-(5-(3-((2-(3-carboxypropanoyl)-4-chloro-6- |
| | methoxyisoindolin-5-yl)oxy)propoxy)-6-methoxybenzo[b] |
| | thiophen-2-yl)-4-oxobutanoic acid |
| 15. | 4-(5-(3-((2-(3-carboxypropanoyl)-5-methoxythieno[3,2-b] |
| | pyridin-6-yl)oxy)propoxy)-6-methoxyisoindolin-2-yl)-4- |
| | oxobutanoic acid |
| 16. | 4-(5-((2-(((2-(3-carboxypropanoyl)-4-chloro-6- |
| | methoxyisoindolin-5-yl)oxy)methyl)allyl)oxy)-4-fluoro- |
| | 6-methoxybenzo[b]thiophen-2-yl)-4-oxobutanoic acid |
| 17. | (S)-4-(5-(3-((2-((S)-3-carboxybutanoyl)-4-fluoro-6- |
| | methoxybenzo[b]thiophen-5-yl)oxy)propoxy)-4-fluoro-6- |
| | methoxyisoindolin-2-yl)-2-methyl-4-oxobutanoic acid |
| 18. | 4-(5-(3-((2-((2-carboxyethyl)carbamoyl)-6-methoxybenzo[b] |
| | thiophen-5-yl)oxy)propoxy)-6-methoxyisoindolin-2-yl)-4- |
| | oxobutanoic acid |
| 19. | 4-(5-((2-(((2-(3-carboxypropanoyl)-6-methoxybenzo[b] |
| | thiophen-5-yl)oxy)methyl)allyl)oxy)-6-methoxyisoindolin-2- |
| | yl)-4-oxobutanoic acid |
| 20. | 4-(5-(3-((2-(3-carboxypropanoyl)-4-fluoro-6-methoxybenzo[b] |
| | thiophen-5-yl)oxy)propoxy)-6-methoxyisoindolin-2-yl)-4- |
| | oxobutanoic acid |
| 21. | trans-2-(5-(3-((2-(3-carboxypropanoyl)-4-fluoro-6- |
| | methoxyisoindolin-5-yl)oxy)propoxy)-6-methoxybenzo[b] |
| | thiophene-2-carbonyl)cyclopropane-1-carboxylic acid |
| 22. | 4-(5-(3-((2-(3-carboxybutanoyl)-6-methoxybenzo[b]thiophen- |
| | 5-yl)oxy)propoxy)-6-methoxyisoindolin-2-yl)-2-methyl-4- |
| | oxobutanoic acid |
| 23. | (S)-4-(5-(3-((2-((S)-3-carboxybutanoyl)-6-methoxybenzo[b] |
| | thiophen-5-yl)oxy)propoxy)-6-methoxyisoindolin-2-yl)-2- |
| | methyl-4-oxobutanoic acid |
| 24. | (S)-4-(5-(3-((2-(3-carboxypropanoyl)-6-methoxyisoindolin-5- |
| | yl)oxy)propoxy)-6-methoxybenzo[b]thiophen-2-yl)-2-methyl- |
| | 4-oxobutanoic acid |
| 25. | (S)-4-(5-(3-((2-(3-carboxypropanoyl)-6-methoxybenzo[b] |
| | thiophen-5-yl)oxy)propoxy)-6-methoxyisoindolin-2-yl)-2- |
| | methyl-4-oxobutanoic acid |
| 26. | (S)-4-(5-(2-((2-((S)-3-carboxybutanoyl)-6-methoxybenzo[b] |
| | thiophen-5-yl)oxy)ethoxy)-6-methoxyisoindolin-2-yl)-2- |
| | methyl-4-oxobutanoic acid |
| 27. | (S)-4-(5-(4-((2-((S)-3-carboxybutanoyl)-6-methoxybenzo[b] |
| | thiophen-5-yl)oxy)butoxy)-6-methoxyisoindolin-2-yl)-2- |
| | methyl-4-oxobutanoic acid |
| 28. | (S)-4-(5-((5-((2-((S)-3-carboxybutanoyl)-6-methoxybenzo[b] |
| | thiophen-5-yl)oxy)pentyl)oxy)-6-methoxyisoindolin-2-yl)-2- |
| | methyl-4-oxobutanoic acid |
| 29. | (S)-4-(5-(4-(2-((S)-3-carboxybutanoyl)-6-methoxybenzo[b] |
| | thiophen-5-yl)butoxy)-6-methoxyisoindolin-2-yl)-2-methyl-4- |
| | oxobutanoic acid |
| 30. | (S)-4-(5-(3-((2-((S)-3-carboxybutanoyl)-6-methoxybenzo[b] |
| | thiophen-5-yl)amino)propoxy)-6-methoxyisoindolin-2-yl)-2- |
| | methyl-4-oxobutanoic acid |
| 31. | (S)-4-(5-(4-((2-((S)-3-carboxybutanoyl)-6-methoxybenzo[b] |
| | thiophen-5-yl)amino)butyl)-6-methoxyisoindolin-2-yl)-2- |
| | methyl-4-oxobutanoic acid |
| 32. | (S)-4-(5-(4-(2-((S)-3-carboxybutanoyl)-6-methoxybenzo[b] |
| | thiophen-5-yl)butyl)-6-methoxyisoindolin-2-yl)-2-methyl-4- |
| | oxobutanoic acid |
| 33. | (S)-4-(5-(5-(2-((S)-3-carboxybutanoyl)-6-methoxybenzo[b] |
| | thiophen-5-yl)pentyl)-6-methoxyisoindolin-2-yl)-2-methyl-4- |
| | oxobutanoic acid |
| 34. | (2S)-4-(5-((4-((2-((S)-3-carboxybutanoyl)-6-methoxybenzo[b] |
| | thiophen-5-yl)oxy)pentan-2-yl)oxy)-6-methoxyisoindolin-2- |
| | yl)-2-methyl-4-oxobutanoic acid |
| 35. | (S)-4-(5-(3-((2-((S)-3-carboxybutanoyl)-6-methoxybenzo[b] |
| | thiophen-5-yl)oxy)-2,2-dimethylpropoxy)-6- |
| | methoxyisoindolin-2-yl)-2-methyl-4-oxobutanoic acid |
| 36. | (S)-4-(5-((1-(((2-((S)-3-carboxybutanoyl)-6-methoxybenzo[b] |
| | thiophen-5-yl)oxy)methyl)cyclopropyl)methoxy)-6- |
| | methoxyisoindolin-2-yl)-2-methyl-4-oxobutanoic acid |
| 37. | 4-(5-(3-((2-(3-carboxypropanoyl)-6-methoxyisoindolin-5-yl) |
| | oxy)propoxy)-6-methoxybenzo[b]thiophen-2-yl)-2,2-dimethyl- |
| | 4-oxobutanoic acid |
| 38. | 4-(5-(3-((2-(3-carboxypropanoyl)-6-methoxybenzo[b]thiophen- |
| | 5-yl)oxy)propoxy)-6-methoxyisoindolin-2-yl)-2,2-dimethyl-4- |
| | oxobutanoic acid |
| 39. | 4-(5-(3-((2-(3-carboxy-3-methylbutanoyl)-6-methoxybenzo[b] |
| | thiophen-5-yl)oxy)propoxy)-6-methoxyisoindolin-2-yl)-2,2- |
| | dimethyl-4-oxobutanoic acid |
| 40. | (S)-4-(5-((4-(2-((S)-3-carboxybutanoyl)-6-methoxybenzo[b] |
| | thiophen-5-yl)butyl)amino)-6-methoxyisoindolin-2-yl)-2- |
| | methyl-4-oxobutanoic acid |
| 41. | (S)-4-(5-((3-((2-((S)-3-carboxybutanoyl)-6-methoxybenzo[b] |
| | thiophen-5-yl)oxy)propyl)amino)-6-methoxyisoindolin-2-yl)- |
| | 2-methyl-4-oxobutanoic acid |
| 42. | (S)-4-(5-(4-((2-((S)-3-carboxybutanoyl)-6-methoxybenzo[b] |
| | thiophen-5-yl)oxy)butyl)-6-methoxyisoindolin-2-yl)-2-methyl- |
| | 4-oxobutanoic acid |
| 43. | (S)-4-(5-(3-((2-((S)-3-carboxybutanoyl)-4-fluoro-6- |
| | methoxybenzo[b]thiophen-5-yl)oxy)propoxy)-6- |
| | methoxyisoindolin-2-yl)-2-methyl-4-oxobutanoic acid |
| 44. | (S)-4-(5-(3-((2-((S)-3-carboxybutanoyl)-4-chloro-6- |
| | methoxybenzo[b]thiophen-5-yl)oxy)propoxy)-6- |
| | methoxyisoindolin-2-yl)-2-methyl-4-oxobutanoic acid |
| 45. | (S)-4-(5-(3-((4-bromo-2-((S)-3-carboxybutanoyl)-6- |
| | methoxybenzo[b]thiophen-5-yl)oxy)propoxy)-6- |
| | methoxyisoindolin-2-yl)-2-methyl-4-oxobutanoic acid |
| 46. | (S)-4-(5-(3-((2-((S)-3-carboxybutanoyl)-6-methoxybenzo[d] |
| | thiazol-5-yl)oxy)propoxy)-6-methoxyisoindolin-2-yl)-2- |
| | methyl-4-oxobutanoic acid |
| 47. | (S)-4-(5-(3-((2-((S)-3-carboxybutanoyl)-6-hydroxybenzo[b] |
| | thiophen-5-yl)oxy)propoxy)-6-methoxyisoindolin-2-yl)-2- |
| | methyl-4-oxobutanoic acid |
| 48. | (S)-4-(5-(3-((2-((S)-3-carboxybutanoyl)-4,7-difluoro-6- |
| | methoxybenzo[b]thiophen-5-yl)oxy)propoxy)-6- |
| | methoxyisoindolin-2-yl)-2-methyl-4-oxobutanoic acid |
| 49. | (S)-4-(5-(3-((2-((S)-3-carboxybutanoyl)-4,7-dichloro-6- |
| | methoxybenzo[b]thiophen-5-yl)oxy)propoxy)-6- |
| | methoxyisoindolin-2-yl)-2-methyl-4-oxobutanoic acid |
| 50. | (2S)-4-(5-(3-((2-((S)-3-carboxybutanoyl)-6-methoxy-3- |
| | methylisoindolin-5-yl)oxy)propoxy)-6-methoxybenzo[b] |
| | thiophen-2-yl)-2-methyl-4-oxobutanoic acid |
| 51. | (S)-4-(5-(3-((2-((S)-3-carboxybutanoyl)-4-fluoro-6- |
| | methoxyisoindolin-5-yl)oxy)propoxy)-6-methoxybenzo[b] |
| | thiophen-2-yl)-2-methyl-4-oxobutanoic acid |
| 52. | (S)-4-(5-(3-((2-((S)-3-carboxybutanoyl)-4-chloro-6- |
| | methoxyisoindolin-5-yl)oxy)propoxy)-6-methoxybenzo[b] |
| | thiophen-2-yl)-2-methyl-4-oxobutanoic acid |
| 53. | (S)-4-(5-(3-((4-bromo-2-((S)-3-carboxybutanoyl)-6- |
| | methoxyisoindolin-5-yl)oxy)propoxy)-6-methoxybenzo[b] |
| | thiophen-2-yl)-2-methyl-4-oxobutanoic acid |
| 54. | (S)-4-(5-(3-((2-((S)-3-carboxybutanoyl)-6-hydroxyisoindolin- |
| | 5-yl)oxy)propoxy)-6-methoxybenzo[b]thiophen-2-yl)-2- |
| | methyl-4-oxobutanoic acid |
| 55. | (S)-4-(6-(3-((2-((S)-3-carboxybutanoyl)-6-methoxybenzo[b] |
| | thiophen-5-yl)oxy)propoxy)-4-fluoro-5-methoxyisoindolin-2- |
| | yl)-2-methyl-4-oxobutanoic acid |
| 56. | (S)-4-(6-(3-((2-((S)-3-carboxybutanoyl)-6-methoxybenzo[b] |
| | thiophen-5-yl)oxy)propoxy)-4-chloro-5-methoxyisoindolin-2- |
| | yl)-2-methyl-4-oxobutanoic acid |
| 57. | (S)-4-(5-(3-((7-bromo-2-((S)-3-carboxybutanoyl)-6- |
| | methoxyisoindolin-5-yl)oxy)propoxy)-6-methoxybenzo[b] |
| | thiophen-2-yl)-2-methyl-4-oxobutanoic acid |
| 58. | (S)-4-(5-(3-((2-((S)-3-carboxybutanoyl)-4,7-difluoro-6- |
| | methoxyisoindolin-5-yl)oxy)propoxy)-6-methoxybenzo[b] |
| | thiophen-2-yl)-2-methyl-4-oxobutanoic acid |
| 59. | (S)-4-(5-(3-((2-((S)-3-carboxybutanoyl)-6-methoxybenzo[b] |
| | thiophen-5-yl)oxy)propoxy)-7-chloro-4-fluoro-6- |
| | methoxyisoindolin-2-yl)-2-methyl-4-oxobutanoic acid |
| 60. | (S)-4-(5-(3-((2-((S)-3-carboxybutanoyl)-4,7-dichloro-6- |
| | methoxyisoindolin-5-yl)oxy)propoxy)-6-methoxybenzo[b] |
| | thiophen-2-yl)-2-methyl-4-oxobutanoic acid |
| 61. | (S)-4-(5-(3-((2-((S)-3-carboxybutanoyl)-4-chloro-6- |
| | methoxybenzo[b]thiophen-5-yl)oxy)propoxy)-4-fluoro-6- |
| | methoxyisoindolin-2-yl)-2-methyl-4-oxobutanoic acid |
| 62. | (S)-4-(5-(3-((2-((S)-3-carboxybutanoyl)-4-chloro-6- |
| | methoxyisoindolin-5-yl)oxy)propoxy)-4-fluoro-6- |
| | methoxybenzo[b]thiophen-2-yl)-2-methyl-4-oxobutanoic acid |
| 63. | (S)-4-(5-(3-((2-((S)-3-carboxybutanoyl)-4-chloro-6- |
| | methoxybenzo[b]thiophen-5-yl)oxy)propoxy)-4-chloro-6- |
| | methoxyisoindolin-2-yl)-2-methyl-4-oxobutanoic acid |
| 64. | (S)-4-(6-(3-((2-((S)-3-carboxybutanoyl)-7-fluoro-6- |
| | methoxybenzo[b]thiophen-5-yl)oxy)propoxy)-4-fluoro-5- |
| | methoxyisoindolin-2-yl)-2-methyl-4-oxobutanoic acid |
| 65. | (S)-4-(6-(3-((2-((S)-3-carboxybutanoyl)-7-chloro-6- |
| | methoxybenzo[b]thiophen-5-yl)oxy)propoxy)-4-fluoro-5- |
| | methoxyisoindolin-2-yl)-2-methyl-4-oxobutanoic acid |
| 66. | (S)-4-(5-(3-((2-((S)-3-carboxybutanoyl)-7-chloro-6- |
| | methoxyisoindolin-5-yl)oxy)propoxy)-7-fluoro-6- |
| | methoxybenzo[b]thiophen-2-yl)-2-methyl-4-oxobutanoic acid |
| 67. | (S)-4-(5-(3-((2-((S)-3-carboxybutanoyl)-4,7-difluoro-6- |
| | methoxyisoindolin-5-yl)oxy)propoxy)-7-chloro-6- |
| | methoxybenzo[b]thiophen-2-yl)-2-methyl-4-oxobutanoic acid |
| 68. | (S)-4-(5-(3-((2-((S)-3-carboxybutanoyl)-4,7-difluoro-6- |
| | methoxyisoindolin-5-yl)oxy)propoxy)-7-fluoro-6- |
| | methoxybenzo[b]thiophen-2-yl)-2-methyl-4-oxobutanoic acid |
| 69. | (S)-4-(5-(3-((2-((S)-3-carboxybutanoyl)-4-chloro-7-fluoro-6- |
| | methoxyisoindolin-5-yl)oxy)propoxy)-7-fluoro-6- |
| | methoxybenzo[b]thiophen-2-yl)-2-methyl-4-oxobutanoic acid |
| 70. | (S)-4-(6-(3-((2-((S)-3-carboxybutanoyl)-4,7-difluoro-6- |
| | methoxybenzo[b]thiophen-5-yl)oxy)propoxy)-4-fluoro-5- |
| | methoxyisoindolin-2-yl)-2-methyl-4-oxobutanoic acid |
| 71. | (S)-4-(6-(3-((2-((S)-3-carboxybutanoyl)-7-chloro-4-fluoro-6- |
| | methoxybenzo[b]thiophen-5-yl)oxy)propoxy)-4-fluoro-5- |
| | methoxyisoindolin-2-yl)-2-methyl-4-oxobutanoic acid |
| 72. | (S)-4-(6-(3-((2-((S)-3-carboxybutanoyl)-4,7-dichloro-6- |
| | methoxybenzo[b]thiophen-5-yl)oxy)propoxy)-4-fluoro-5- |
| | methoxyisoindolin-2-yl)-2-methyl-4-oxobutanoic acid |
| 73. | (S)-4-(5-(3-((2-((S)-3-carboxybutanoyl)-4-chloro-7-fluoro-6- |
| | methoxyisoindolin-5-yl)oxy)propoxy)-7-chloro-6- |
| | methoxybenzo[b]thiophen-2-yl)-2-methyl-4-oxobutanoic acid |
| 74. | (S)-4-(5-(3-((2-((S)-3-carboxybutanoyl)-4,7-dichloro-6- |
| | methoxyisoindolin-5-yl)oxy)propoxy)-7-fluoro-6- |
| | methoxybenzo[b]thiophen-2-yl)-2-methyl-4-oxobutanoic acid |
| 75. | (S)-4-(5-(3-((2-((S)-3-carboxybutanoyl)-7-chloro-4-fluoro-6- |
| | methoxyisoindolin-5-yl)oxy)propoxy)-7-fluoro-6- |
| | methoxybenzo[b]thiophen-2-yl)-2-methyl-4-oxobutanoic acid |
| 76. | (S)-4-(6-(3-((2-((S)-3-carboxybutanoyl)-4,7-difluoro-6- |
| | methoxybenzo[b]thiophen-5-yl)oxy)propoxy)-4-chloro-5- |
| | methoxyisoindolin-2-yl)-2-methyl-4-oxobutanoic acid |
| 77. | (S)-4-(6-(3-((2-((S)-3-carboxybutanoyl)-4-chloro-7-fluoro-6- |
| | methoxybenzo[b]thiophen-5-yl)oxy)propoxy)-4-chloro-5- |
| | methoxyisoindolin-2-yl)-2-methyl-4-oxobutanoic acid |
| 78. | (S)-4-(5-(3-((2-((S)-3-carboxybutanoyl)-4-fluoro-6- |
| | hydroxyisoindolin-5-yl)oxy)propoxy)-4-fluoro-6- |
| | methoxybenzo[b]thiophen-2-yl)-2-methyl-4-oxobutanoic acid |
| 79. | (S)-4-(5-(3-((2-((S)-3-carboxybutanoyl)-4-chloro-6- |
| | methoxybenzo[b]thiophen-5-yl)oxy)propoxy)-4-fluoro-6- |
| | hydroxyisoindolin-2-yl)-2-methyl-4-oxobutanoic acid |
| 80. | (S)-4-(5-(3-((2-((S)-3-carboxybutanoyl)-4-chloro-6- |
| | hydroxyisoindolin-5-yl)oxy)propoxy)-4-fluoro-6- |
| | methoxybenzo[b]thiophen-2-yl)-2-methyl-4-oxobutanoic acid |
| 81. | (S)-4-(5-(3-((2-((S)-3-carboxybutanoyl)-4-chloro-6- |
| | hydroxyisoindolin-5-yl)oxy)propoxy)-4-chloro-6- |
| | methoxybenzo[b]thiophen-2-yl)-2-methyl-4-oxobutanoic acid |
| 82. | (S)-4-(6-(3-((2-((S)-3-carboxybutanoyl)-6- |
| | methoxybenzo[b]thiophen-5-yl)oxy)propoxy)-4-fluoro-5- |
| | hydroxyisoindolin-2-yl)-2-methyl-4-oxobutanoic acid |
| 83. | (S)-4-(5-(3-((2-((S)-3-carboxybutanoyl)-7-fluoro-6- |
| | hydroxyisoindolin-5-yl)oxy)propoxy)-7-fluoro-6- |
| | methoxybenzo[b]thiophen-2-yl)-2-methyl-4-oxobutanoic acid |
| 84. | (S)-4-(6-(3-((2-((S)-3-carboxybutanoyl)-7-chloro-6- |
| | methoxybenzo[b]thiophen-5-yl)oxy)propoxy)-4-fluoro-5- |
| | hydroxyisoindolin-2-yl)-2-methyl-4-oxobutanoic acid |
| 85. | (S)-4-(5-(3-((2-((S)-3-carboxybutanoyl)-7-chloro-6- |
| | hydroxyisoindolin-5-yl)oxy)propoxy)-7-fluoro-6- |
| | methoxybenzo[b]thiophen-2-yl)-2-methyl-4-oxobutanoic acid |
| 86. | (S)-4-(5-(3-((2-((S)-3-carboxybutanoyl)-4,7-difluoro-6- |
| | methoxyisoindolin-5-yl)oxy)propoxy)-4-chloro-7-fluoro-6- |
| | methoxybenzo[b]thiophen-2-yl)-2-methyl-4-oxobutanoic acid |
| 87. | (S)-4-(5-(3-((2-((S)-3-carboxybutanoyl)-4-chloro-7-fluoro-6- |
| | methoxybenzo[b]thiophen-5-yl)oxy)propoxy)-4-chloro-7- |
| | fluoro-6-methoxyisoindolin-2-yl)-2-methyl-4-oxobutanoic |
| | acid |
| 88. | (S)-4-(5-(3-((2-((S)-3-carboxybutanoyl)-4,7-difluoro-6- |
| | methoxybenzo[b]thiophen-5-yl)oxy)propoxy)-4,7-difluoro-6- |
| | methoxyisoindolin-2-yl)-2-methyl-4-oxobutanoic acid |
| 89. | (S)-4-(5-(3-((2-((S)-3-carboxybutanoyl)-4,7-difluoro-6- |
| | methoxyisoindolin-5-yl)oxy)propoxy)-7-chloro-4-fluoro-6- |
| | methoxybenzo[b]thiophen-2-yl)-2-methyl-4-oxobutanoic acid |
| 90. | (S)-4-(5-(3-((2-((S)-3-carboxybutanoyl)-4,7-dichloro-6- |
| | methoxybenzo[b]thiophen-5-yl)oxy)propoxy)-4,7-difluoro-6- |
| | methoxyisoindolin-2-yl)-2-methyl-4-oxobutanoic acid |
| 91. | (S)-4-(5-(3-((2-((S)-3-carboxybutanoyl)-4,7-dichloro-6- |
| | methoxybenzo[b]thiophen-5-yl)oxy)propoxy)-4-chloro-7- |
| | fluoro-6-methoxyisoindolin-2-yl)-2-methyl-4-oxobutanoic |
| | acid |
| 92. | (S)-4-(5-(3-((2-((S)-3-carboxybutanoyl)-4-chloro-7-fluoro-6- |
| | methoxyisoindolin-5-yl)oxy)propoxy)-7-chloro-4-fluoro-6- |
| | methoxybenzo[b]thiophen-2-yl)-2-methyl-4-oxobutanoicacid |
| 93. | (S)-4-(5-(3-((2-((S)-3-carboxybutanoyl)-4,7-dichloro-6- |
| | methoxyisoindolin-5-yl)oxy)propoxy)-4-chloro-7-fluoro-6- |
| | methoxybenzo[b]thiophen-2-yl)-2-methyl-4-oxobutanoic acid |
| 94. | (S)-4-(5-(3-((2-((S)-3-carboxybutanoyl)-4-chloro-7-fluoro-6- |
| | methoxybenzo[b]thiophen-5-yl)oxy)propoxy)-7-chloro-4- |
| | fluoro-6-methoxyisoindolin-2-yl)-2-methyl-4-oxobutanoic |
| | acid |
| 95. | (2S,2′S)-4,4′-(7,8,11,13,17,18-hexahydro-6H,12H,16H- |
| | thieno[2″,3″:4′,5′]benzo[1′,2′:9,10][1,4,8,11] |
| | tetraoxacyclotetradecino[2,3-f]isoindole-2,12-diyl)bis(2- |
| | methyl-4-oxobutanoic acid) |
| 96. | (S)-4-(6-methoxy-5-(3-((6-methoxy-2-((S)-3-methyl-4- |
| | (methylsulfonamido)-4-oxobutanoyl)isoindolin-5-yl)oxy) |
| | propoxy)benzo[b]thiophen-2-yl)-2-methyl-4-oxobutanoic acid |
| 97. | (S)-4-(5-(3-((2-((S)-4-((N,N-dimethylsulfamoyl)amino)-3- |
| | methyl-4-oxobutanoyl)-6-methoxyisoindolin-5-yl)oxy) |
| | propoxy)-6-methoxybenzo[b]thiophen-2-yl)-2-methyl-4- |
| | oxobutanoic acid |
| 98. | (S)-4-(5-(3-((2-(3-cyanopropanoyl)-6-methoxyisoindolin-5- |
| | yl)oxy)propoxy)-6-methoxybenzo[b]thiophen-2-yl)-2-methyl- |
| | 4-oxobutanoic acid |
| 99. | 4-(5-(3-((2-(3-cyanopropanoyl)-6-methoxybenzo[b]thiophen- |
| | 5-yl)oxy)propoxy)-6-methoxyisoindolin-2-yl)-4- |
| | oxobutanenitrile |
| 100. | (S)-4-(5-(3-((2-(3-cyanopropanoyl)-6-methoxybenzo[b] |
| | thiophen-5-yl)oxy)propoxy)-6-methoxyisoindolin-2-yl)-2- |
| | methyl-4-oxobutanoic acid |
| 101. | (1R,2R)-2-(5-(3-((2-((S)-3-carboxybutanoyl)-6- |
| | methoxyisoindolin-5-yl)oxy)propoxy)-6-methoxybenzo[b] |
| | thiophene-2-carbonyl)cyclobutane-1-carboxylic acid |
| 102. | (1R,2R)-2-(5-(3-((2-((1R,2R)-2-carboxycyclobutane-1- |
| | carbonyl)-6-methoxybenzo[b]thiophen-5-yl)oxy)propoxy)-6- |
| | methoxyisoindoline-2-carbonyl)cyclobutane-1-carboxylic acid |
| 103. | (1R,2R)-2-(5-(3-((2-((S)-3-carboxybutanoyl)-6- |
| | methoxybenzo[b]thiophen-5-yl)oxy)propoxy)-6- |
| | methoxyisoindoline-2-carbonyl)cyclobutane-1-carboxylic acid |
| 104. | (1R,2R)-2-(5-(3-((2-((S)-3-carboxybutanoyl)-6- |
| | methoxyisoindolin-5-yl)oxy)propoxy)-6-methoxybenzo[b] |
| | thiophene-2-carbonyl)cyclopropane-1-carboxylic acid |
| 105. | (1R,2R)-2-(5-(3-((2-((1R,2R)-2-carboxycyclopropane-1- |
| | carbonyl)-6-methoxybenzo[b]thiophen-5-yl)oxy)propoxy)-6- |
| | methoxyisoindoline-2-carbonyl)cyclopropane-1-carboxylic |
| | acid |
| 106. | (1R,2R)-2-(5-(3-((2-((S)-3-carboxybutanoyl)-6-methoxybenzo |
| | [b]thiophen-5-yl)oxy)propoxy)-6-methoxyisoindoline-2- |
| | carbonyl)cyclopropane-1-carboxylic acid |
| 107. | (S)-N-(N,N-dimethylsulfamoyl)-4-(5-(3-((2-((S)-4-((N,N- |
| | dimethylsulfamoyl)amino)-3-methyl-4-oxobutanoyl)-6- |
| | methoxybenzo[b]thiophen-5-yl)oxy)propoxy)-6- |
| | methoxyisoindolin-2-yl)-2-methyl-4-oxobutanamide |
| 108. | (S)-4-(5-(3-((2-((S)-4-((N,N-dimethylsulfamoyl)amino)-3- |
| | methyl-4-oxobutanoyl)-6-methoxybenzo[b]thiophen-5-yl) |
| | oxy)propoxy)-6-methoxyisoindolin-2-yl)-2-methyl-4- |
| | oxobutanoic acid |
| 109. | (S)-4-(6-methoxy-5-(3-((6-methoxy-2-((S)-3-methyl-4- |
| | (methylsulfonamido)-4-oxobutanoyl)isoindolin-5-yl)oxy) |
| | propoxy)benzo[b]thiophen-2-yl)-2-methyl-N-(methylsulfonyl)- |
| | 4-oxobutanamide |
| 110. | (S)-4-(5-methoxy-6-(3-((6-methoxy-2-((S)-3-methyl-4- |
| | (methylsulfonamido)-4-oxobutanoyl)benzo[b]thiophen-5-yl) |
| | oxy)propoxy)isoindolin-2-yl)-2-methyl-4-oxobutanoic acid |
| 111. | (S)-4-(4-(3-((2-((S)-3-carboxybutanoyl)-6-methoxybenzo[b] |
| | thiophen-5-yl)oxy)propoxy)-6-methoxyisoindolin-2-yl)-2- |
| | methyl-4-oxobutanoic acid |
| 112. | (S)-4-(5-(3-((2-((S)-3-carboxybutanoyl)-6-methoxybenzo[b] |
| | thiophen-4-yl)oxy)propoxy)-6-methoxyisoindolin-2-yl)-2- |
| | methyl-4-oxobutanoic acid |
| 113. | (S)-4-(4-(4-((2-((S)-3-carboxybutanoyl)-6-methoxybenzo[b] |
| | thiophen-5-yl)oxy)butyl)-6-methoxyisoindolin-2-yl)-2-methyl- |
| | 4-oxobutanoic acid |
| 114 | (S)-4-(4-(4-(2-((S)-3-carboxybutanoyl)-6-methoxybenzo[b] |
| | thiophen-5-yl)butoxy)-6-methoxyisoindolin-2-yl)-2-methyl-4- |
| | oxobutanoic acid |
| 115. | (S)-4-(5-(4-((2-((S)-3-carboxybutanoyl)-6-methoxybenzo[b] |
| | thiophen-4-yl)oxy)butyl)-6-methoxyisoindolin-2-yl)-2-methyl- |
| | 4-oxobutanoic acid |
| 116. | (S)-4-(5-(4-(2-((S)-3-carboxybutanoyl)-6-methoxybenzo[b] |
| | thiophen-4-yl)butoxy)-6-methoxyisoindolin-2-yl)-2-methyl-4- |
| | oxobutanoic acid |
| 117. | (S)-4-(5-(3-((2-((S)-3-carboxybutanoyl)-6-methoxyisoindolin- |
| | 5-yl)oxy)propoxy)-6-methylbenzo[b]thiophen-2-yl)-2-methyl- |
| | 4-oxobutanoic acid |
| 118. | (S)-4-(5-(3-((2-((S)-3-carboxybutanoyl)-6-ethylbenzo[b] |
| | thiophen-5-yl)oxy)propoxy)-6-methoxyisoindolin-2-yl)-2- |
| | methyl-4-oxobutanoic acid |
| 119. | (S)-4-(5-(3-((2-((S)-3-carboxybutanoyl)-6-methoxyisoindolin- |
| | 5-yl)oxy)propoxy)-6-vinylbenzo[b]thiophen-2-yl)-2-methyl-4- |
| | oxobutanoic acid |
| 120. | (S)-4-(5-(3-((2-((S)-3-carboxybutanoyl)-6-ethynylbenzo[b] |
| | thiophen-5-yl)oxy)propoxy)-6-methoxyisoindolin-2-yl)-2- |
| | methyl-4-oxobutanoic acid |
| 121. | (S)-4-(5-(3-((6-amino-2-((S)-3-carboxybutanoyl)benzo[b] |
| | thiophen-5-yl)oxy)propoxy)-6-methoxyisoindolin-2-yl)-2- |
| | methyl-4-oxobutanoic acid |
| 122. | (S)-4-(5-(3-((2-((S)-3-carboxybutanoyl)-6-(methylamino) |
| | benzo[b]thiophen-5-yl)oxy)propoxy)-6-methoxyisoindolin- |
| | 2-2-yl)-methyl-4-oxobutanoic acid |
| 123. | (S)-4-(5-(3-((6-bromo-2-((S)-3-carboxybutanoyl)benzo[b] |
| | thiophen-5-yl)oxy)propoxy)-6-methoxyisoindolin-2-yl)-2- |
| | methyl-4-oxobutanoic acid |
| 124. | (S)-4-(5-(3-((2-((S)-3-carboxybutanoyl)-6-methoxybenzo[b] |
| | thiophen-5-yl)oxy)propoxy)-6-methylisoindolin-2-yl)-2- |
| | methyl-4-oxobutanoic acid |
| 125. | (S)-4-(5-(3-((2-((S)-3-carboxybutanoyl)-6-ethylisoindolin-5- |
| | yl)oxy)propoxy)-6-methoxybenzo[b]thiophen-2-yl)-2-methyl- |
| | 4-oxobutanoic acid |
| 126. | (S)-4-(5-(3-((2-((S)-3-carboxybutanoyl)-6-methoxybenzo[b] |
| | thiophen-5-yl)oxy)propoxy)-6-vinylisoindolin-2-yl)-2-methyl- |
| | 4-oxobutanoic acid |
| 127. | (S)-4-(5-(3-((2-((S)-3-carboxybutanoyl)-6-ethynylisoindolin-5- |
| | yl)oxy)propoxy)-6-methoxybenzo[b]thiophen-2-yl)-2-methyl- |
| | 4-oxobutanoic acid |
| 128. | (S)-4-(5-(3-((6-amino-2-((S)-3-carboxybutanoyl)isoindolin-5- |
| | yl)oxy)propoxy)-6-methoxybenzo[b]thiophen-2-yl)-2-methyl- |
| | 4-oxobutanoic acid |
| 129. | (S)-4-(5-(3-((2-((S)-3-carboxybutanoyl)-6-(methylamino) |
| | isoindolin-5-yl)oxy)propoxy)-6-methoxybenzo[b]thiophen-2- |
| | yl)-2-methyl-4-oxobutanoic acid |
| 130. | (S)-4-(5-(3-((6-bromo-2-((S)-3-carboxybutanoyl)isoindolin-5- |
| | yl)oxy)propoxy)-6-methoxybenzo[b]thiophen-2-yl)-2-methyl- |
| | 4-oxobutanoic acid |
| 131. | (S)-4-(5-(3-((6-((S)-3-carboxybutanoyl)-3-methoxy-6,7- |
| | dihydro-5H-pyrrolo[3,4-b]pyridin-2-yl)oxy)propoxy)-6- |
| | methoxybenzo[b]thiophen-2-yl)-2-methyl-4-oxobutanoic acid |
| 132. | (S)-4-(5-(3-((6-((S)-3-carboxybutanoyl)-3-methoxy-6,7- |
| | dihydro-5H-pyrrolo[3,4-b]pyridin-2-yl)oxy)propoxy)-4-fluoro- |
| | 6-methoxybenzo[b]thiophen-2-yl)-2-methyl-4-oxobutanoic |
| | acid |
| 133. | (S)-4-(5-(3-((6-((S)-3-carboxybutanoyl)-3-methoxy-6,7- |
| | dihydro-5H-pyrrolo[3,4-b]pyridin-2-yl)oxy)propoxy)-4- |
| | chloro-6-methoxybenzo[b]thiophen-2-yl)-2-methyl-4- |
| | oxobutanoic acid |
| 134. | (S)-4-(5-(3-((6-((S)-3-carboxybutanoyl)-3-hydroxy-6,7- |
| | dihydro-5H-pyrrolo[3,4-b]pyridin-2-yl)oxy)propoxy)-6- |
| | methoxythieno[3,2-b]pyridin-2-yl)-2-methyl-4-oxobutanoic |
| | acid |
| 135. | (S)-4-(5-(3-((6-((S)-3-carboxybutanoyl)-3-methoxy-6,7- |
| | dihydro-5H-pyrrolo[3,4-b]pyridin-2-yl)oxy)propoxy)-6- |
| | hydroxythieno[3,2-b]pyridin-2-yl)-2-methyl-4-oxobutanoic |
| | acid |
| 136. | (S)-4-(5-(3-((6-((S)-3-carboxybutanoyl)-3-methoxy-6,7- |
| | dihydro-5H-pyrrolo[3,4-b]pyridin-2-yl)oxy)propoxy)-6- |
| | methoxythieno[3,2-b]pyridin-2-yl)-2-methyl-4-oxobutanoic |
| | acid |
| 137. | (S)-4-(5-(3-((6-((S)-3-carboxybutanoyl)-4-fluoro-3-methoxy- |
| | 6,7-dihydro-5H-pyrrolo[3,4-b]pyridin-2-yl)oxy)propoxy)-6- |
| | methoxythieno[3,2-b]pyridin-2-yl)-2-methyl-4-oxobutanoic |
| | acid |
| 138. | (S)-4-(5-(3-((6-((S)-3-carboxybutanoyl)-4-chloro-3-methoxy- |
| | 6,7-dihydro-5H-pyrrolo[3,4-b]pyridin-2-yl)oxy)propoxy)-6- |
| | methoxythieno[3,2-b]pyridin-2-yl)-2-methyl-4-oxobutanoic |
| | acid |
| 139. | (S)-4-(5-(3-((2-((S)-3-carboxybutanoyl)-6-methoxyisoindolin- |
| | 5-yl)oxy)propoxy)-6-methoxythieno[3,2-b]pyridin-2-yl)-2- |
| | methyl-4-oxobutanoic acid |
| 140. | (S)-4-(5-(3-((2-((S)-3-carboxybutanoyl)-4-fluoro-6- |
| | methoxyisoindolin-5-yl)oxy)propoxy)-6-methoxythieno |
| | [3,2-b]pyridin-2-yl)-2-methyl-4-oxobutanoic acid |
| 141. | (S)-4-(5-(3-((2-((S)-3-carboxybutanoyl)-4-chloro-6- |
| | methoxyisoindolin-5-yl)oxy)propoxy)-6-methoxythieno |
| | [3,2-b]pyridin-2-yl)-2-methyl-4-oxobutanoic acid |
| 142. | (S)-4-(5-(3-((2-((S)-3-carboxybutanoyl)-4,7-dichloro-6- |
| | methoxyisoindolin-5-yl)oxy)propoxy)-6-methoxythieno |
| | [3,2-b]pyridin-2-yl)-2-methyl-4-oxobutanoic acid |
| 143. | (S)-4-(5-(3-((2-((S)-3-carboxybutanoyl)-4-chloro-7-fluoro-6- |
| | methoxyisoindolin-5-yl)oxy)propoxy)-6-methoxythieno |
| | [3,2-b]pyridin-2-yl)-2-methyl-4-oxobutanoic acid |
| 144. | (S)-4-(5-(3-((2-((S)-3-carboxybutanoyl)-4,7-difluoro-6- |
| | methoxyisoindolin-5-yl)oxy)propoxy)-6-methoxythieno |
| | [3,2-b]pyridin-2-yl)-2-methyl-4-oxobutanoic acid |
| 145. | (S)-4-(5-(3-((2-((S)-3-carboxybutanoyl)-4,7-difluoro-6- |
| | hydroxyisoindolin-5-yl)oxy)propoxy)-6-methoxythieno |
| | [3,2-b]pyridin-2-yl)-2-methyl-4-oxobutanoic acid |
| 146. | (S)-4-(5-(3-((2-((S)-3-carboxybutanoyl)-4-fluoro-6- |
| | hydroxybenzo[b]thiophen-5-yl)oxy)propoxy)-4-fluoro-6- |
| | methoxyisoindolin-2-yl)-2-methyl-4-oxobutanoic acid |
| 147. | (S)-4-(5-(3-((2-((S)-3-carboxybutanoyl)-4-chloro-6- |
| | hydroxybenzo[b]thiophen-5-yl)oxy)propoxy)-4-fluoro-6- |
| | methoxyisoindolin-2-yl)-2-methyl-4-oxobutanoic acid |
| 148. | (S)-4-(5-(3-((2-((S)-3-carboxybutanoyl)-4-chloro-6- |
| | methoxyisoindolin-5-yl)oxy)propoxy)-4-fluoro-6- |
| | hydroxybenzo[b]thiophen-2-yl)-2-methyl-4-oxobutanoic acid |
| 149. | (S)-4-(5-(3-((2-((S)-3-carboxybutanoyl)-4-chloro-6- |
| | hydroxybenzo[b]thiophen-5-yl)oxy)propoxy)-4-chloro-6- |
| | methoxyisoindolin-2-yl)-2-methyl-4-oxobutanoic acid |
| 150. | (S)-4-(6-(3-((2-((S)-3-carboxybutanoyl)-6-hydroxybenzo[b] |
| | fthiophen-5-yl)oxy)propoxy)-4-luoro-5-methoxyisoindolin-2- |
| | yl)-2-methyl-4-oxobutanoic acid |
| 151. | (S)-4-(6-(3-((2-((S)-3-carboxybutanoyl)-7-fluoro-6- |
| | hydroxybenzo[b]thiophen-5-yl)oxy)propoxy)-4-fluoro-5- |
| | methoxyisoindolin-2-yl)-2-methyl-4-oxobutanoic acid |
| 152. | (S)-4-(6-(3-((2-((S)-3-carboxybutanoyl)-7-chloro-6- |
| | hydroxybenzo[b]thiophen-5-yl)oxy)propoxy)-4-fluoro-5- |
| | methoxyisoindolin-2-yl)-2-methyl-4-oxobutanoic acid |
| 153. | (S)-4-(5-(3-((2-((S)-3-carboxybutanoyl)-7-chloro-6- |
| | methoxyisoindolin-5-yl)oxy)propoxy)-7-fluoro-6- |
| | hydroxybenzo[b]thiophen-2-yl)-2-methyl-4-oxobutanoic acid |
| 154. | (S)-4-(5-(3-((2-((S)-3-carboxybutanoyl)-4,7-difluoro-6- |
| | methoxyisoindolin-5-yl)oxy)propoxy)-7-chloro-6- |
| | hydroxybenzo[b]thiophen-2-yl)-2-methyl-4-oxobutanoic acid |
| 155. | (S)-4-(5-(3-((2-((S)-3-carboxybutanoyl)-4,7-difluoro-6- |
| | methoxyisoindolin-5-yl)oxy)propoxy)-7-fluoro-6- |
| | hydroxybenzo[b]thiophen-2-yl)-2-methyl-4-oxobutanoic acid |
| 156. | (S)-4-(5-(3-((2-((S)-3-carboxybutanoyl)-4-chloro-7-fluoro-6- |
| | methoxyisoindolin-5-yl)oxy)propoxy)-7-fluoro-6- |
| | hydroxybenzo[b]thiophen-2-yl)-2-methyl-4-oxobutanoic acid |
| 157. | (S)-4-(6-(3-((2-((S)-3-carboxybutanoyl)-4,7-difluoro-6- |
| | yhydroxybenzo[b]thiophen-5-l)oxy)propoxy)-4-fluoro-5- |
| | methoxyisoindolin-2-yl)-2-methyl-4-oxobutanoic acid |
| 158. | (S)-4-(6-(3-((2-((S)-3-carboxybutanoyl)-7-chloro-4-fluoro-6- |
| | hydroxybenzo[b]thiophen-5-yl)oxy)propoxy)-4-fluoro-5- |
| | methoxyisoindolin-2-yl)-2-methyl-4-oxobutanoic acid |
| 159. | (S)-4-(6-(3-((2-((S)-3-carboxybutanoyl)-4,7-dichloro-6- |
| | hydroxybenzo[b]thiophen-5-yl)oxy)propoxy)-4-fluoro-5- |
| | methoxyisoindolin-2-yl)-2-methyl-4-oxobutanoic acid |
| 160. | (S)-4-(5-(3-((2-((S)-3-carboxybutanoyl)-5-methoxybenzo[b] |
| | thiophen-6-yl)oxy)propoxy)-6-methoxyisoindolin-2-yl)-2- |
| | methyl-4-oxobutanoic acid |
| 161. | 4-(5-(3-((2-(3-carboxypropanoyl)-6-methoxyisoindolin-5-yl) |
| | oxy)propoxy)-6-methoxythieno[2,3-b]pyridin-2-yl)-4- |
| | oxobutanoic acid |
| 162. | 4-(5-(3-((2-(4-(((4R,5R)-5-hydroxy-1,2-dithian-4-yl)oxy)-4- |
| | oxobutanoyl)-6-methoxythieno[2,3-b]pyridin-5-yl)oxy) |
| | propoxy)-6-methoxyisoindolin-2-yl)-4-oxobutanoic acid |
| 163. | 4-(5-(3-((6-(3-carboxypropanoyl)-3-methoxy-6,7-dihydro-5H- |
| | pyrrolo[3,4-b]pyridin-2-yl)oxy)propoxy)-6-methoxybenzo[b] |
| | thiophen-2-yl)-4-oxobutanoic acid |
| 164. | 4-(2-(3-((2-(4-(((4R,5R)-5-hydroxy-1,2-dithian-4-yl)oxy)-4- |
| | oxobutanoyl)-6-methoxybenzo[b]thiophen-5-yl)oxy)propoxy)- |
| | 3-methoxy-5,7-dihydro-6H-pyrrolo[3,4-b]pyridin-6-yl)-4- |
| | oxobutanoic acid |
| 165. | 4-(5-(3-((6-(3-carboxypropanoyl)-2-methoxy-6,7-dihydro-5H- |
| | pyrrolo[3,4-b]pyridin-3-yl)oxy)propoxy)-6-methoxybenzo[b] |
| | thiophen-2-yl)-4-oxobutanoic acid |
| 166. | 4-(3-(3-((2-(4-(((4R,5R)-5-hydroxy-1,2-dithian-4-yl)oxy)-4- |
| | oxobutanoyl)-6-methoxybenzo[b]thiophen-5-yl)oxy)propoxy)- |
| | 2-methoxy-5,7-dihydro-6H-pyrrolo[3,4-b]pyridin-6-yl)-4- |
| | oxobutanoic acid |
| 167. | 4-(2-(3-((2-(4-(((4R,5R)-5-hydroxy-1,2-dithian-4-yl)oxy)-4- |
| | oxobutanoyl)-6-methoxythieno[3,2-b]pyridin-5-yl)oxy) |
| | propoxy)-3-methoxy-5,7-dihydro-6H-pyrrolo[3,4-b]pyridin-6- |
| | yl)-4-oxobutanoic acid |
| 168. | 4-(5-(3-((2-(3-carboxypropanoyl)-5-methoxythieno[2,3-b] |
| | pyridin-6-yl)oxy)propoxy)-6-methoxyisoindolin-2-yl)-4- |
| | oxobutanoic acid |
| 169. | (S)-4-(5-(3-((2-(3-carboxypropanoyl)-5-methoxythieno[2,3-b] |
| | pyridin-6-yl)oxy)propoxy)-6-methoxyisoindolin-2-yl)-2- |
| | methyl-4-oxobutanoic acid |
| 170. | (S)-4-(6-(3-((2-(3-carboxypropanoyl)-6-methoxyisoindolin-5- |
| | yl)oxy)propoxy)-5-methoxythieno[2,3-b]pyridin-2-yl)-2- |
| | methyl-4-oxobutanoic acid |
| 171. | (S)-4-(5-(3-((2-((S)-3-carboxybutanoyl)-5-methoxythieno |
| | [2,3-b]pyridin-6-yl)oxy)propoxy)-6-methoxyisoindolin-2-yl)- |
| | 2-methyl-4-oxobutanoic acid |
| 172. | 4-(5-(3-((2-(4-(((4R,5R)-5-hydroxy-1,2-dithian-4-yl)oxy)-4- |
| | oxobutanoyl)-5-methoxythieno[2,3-b]pyridin-6-yl)oxy) |
| | propoxy)-6-methoxyisoindolin-2-yl)-4-oxobutanoic acid |

In some embodiments, the STING agonist moiety [D] is a compound of Formula Y′-0 or Y′-0′, or an ester, stereoisomer, tautomer, isotopic derivative, prodrug or pharmaceutically acceptable salt thereof:

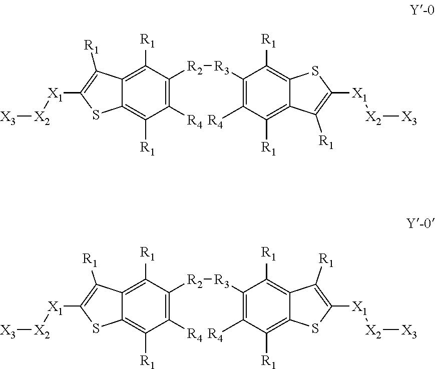

wherein each of R₁, R₂, R₃, R₄, X₁, X₂, and X₃ is the same as defined herein.

In some embodiments, the STING agonist moiety [D] is a compound of Formula Y′-0”, or an ester, stereoisomer, tautomer, isotopic derivative, prodrug or pharmaceutically acceptable salt thereof:

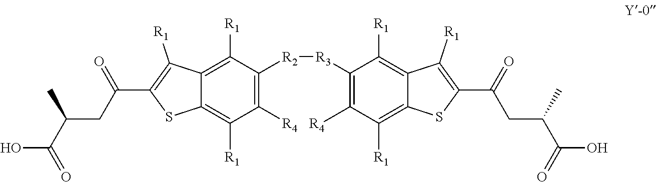

wherein each of R₁, R², R³, and R⁴ is the same as defined herein.

In some embodiments, the STING agonist moiety [D] is a compound of Formula Y′-2, or an ester, stereoisomer, tautomer, isotopic derivative, prodrug or pharmaceutically acceptable salt thereof:

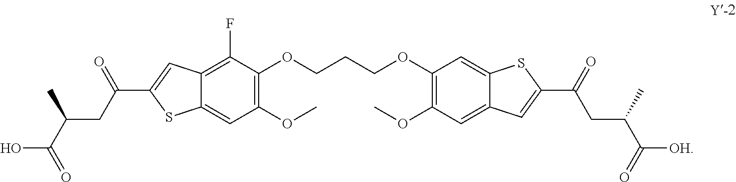 [SMILES: COc1cc2cc(C(=O)C[C@H](C)C(=O)O)sc2cc1OCCCOc1c(OC)cc2sc(C(=O)C[C@H](C)C(=O)O)cc2c1F]

In some embodiments, the STING agonist moiety [D] is a compound of Formula Y′-1, Y′-1′ or Y′-1″, or an ester, stereoisomer, tautomer, isotopic derivative, prodrug or pharmaceutically acceptable salt thereof:

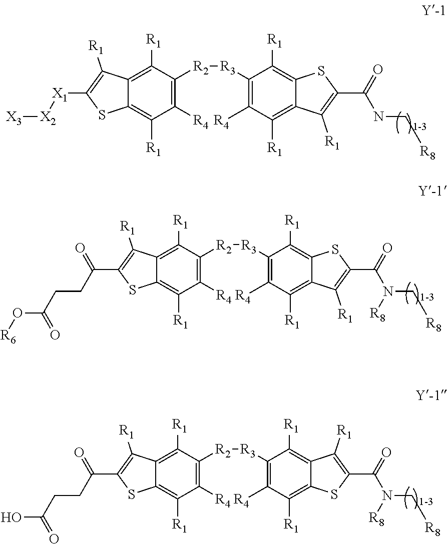

wherein each of R₁, R², R³, R⁴, R₆, Rs, X₁, X2, and X3 is the same as defined herein.

In some embodiments, the STING agonist moiety [D] is a compound of Formula Y′-2′, or an ester, stereoisomer, tautomer, isotopic derivative, prodrug or pharmaceutically acceptable salt thereof:

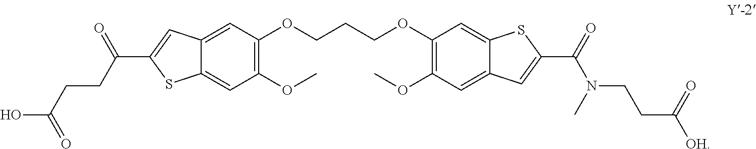 [SMILES: COc1cc2sc(C(=O)CCC(=O)O)cc2cc1OCCCOc1cc2sc(C(=O)N(C)CCC(=O)O)cc2cc1OC]

In some embodiments, the STING agonist moiety [D] is selected from one of the following compounds, or its pharmaceutically acceptable salt:

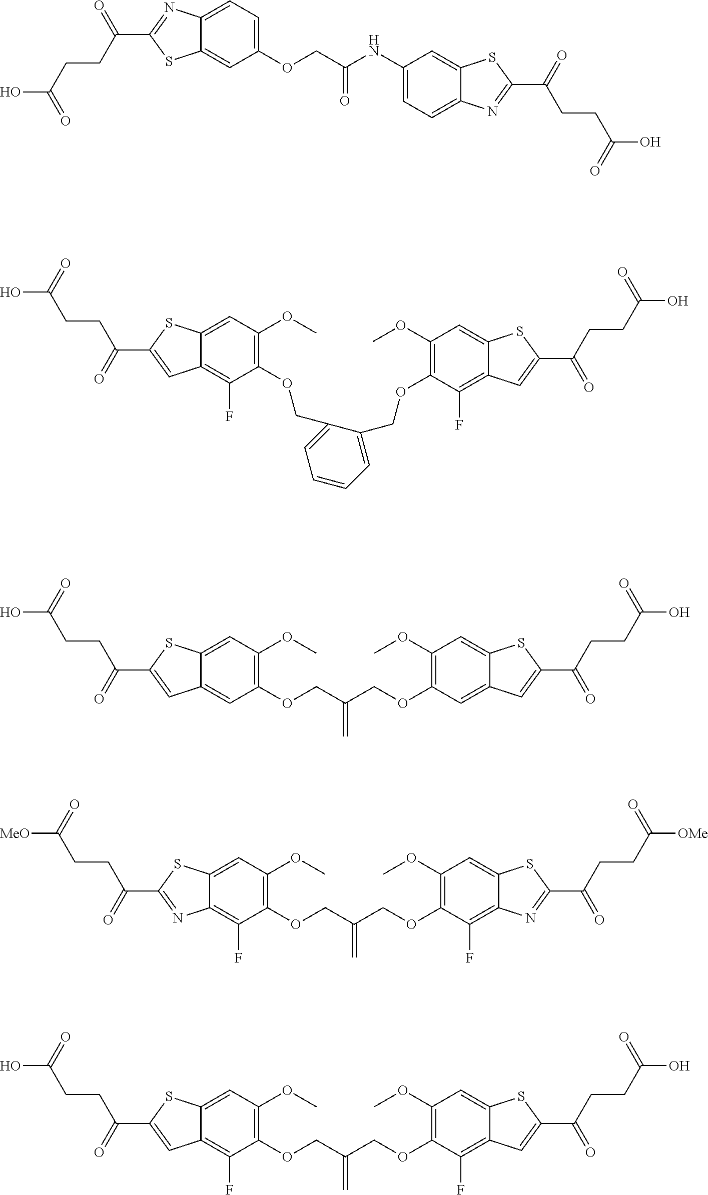 [SMILES: O=C(O)CCC(=O)c1nc2ccc(NC(=O)COc3ccc4nc(C(=O)CCC(=O)O)sc4c3)cc2s1 ; COc1cc2sc(C(=O)CCC(=O)O)cc2c(F)c1OCc1ccccc1COc1c(OC)cc2sc(C(=O)CCC(=O)O)cc2c1F ; C=C(COc1cc2cc(C(=O)CCC(=O)O)sc2cc1OC)COc1cc2cc(C(=O)CCC(=O)O)sc2cc1OC ; C=C(COc1c(OC)cc2sc(C(=O)CCC(=O)OC)nc2c1F)COc1c(OC)cc2sc(C(=O)CCC(=O)OC)nc2c1F ; C=C(COc1c(OC)cc2sc(C(=O)CCC(=O)O)cc2c1F)COc1c(OC)cc2sc(C(=O)CCC(=O)O)cc2c1F]

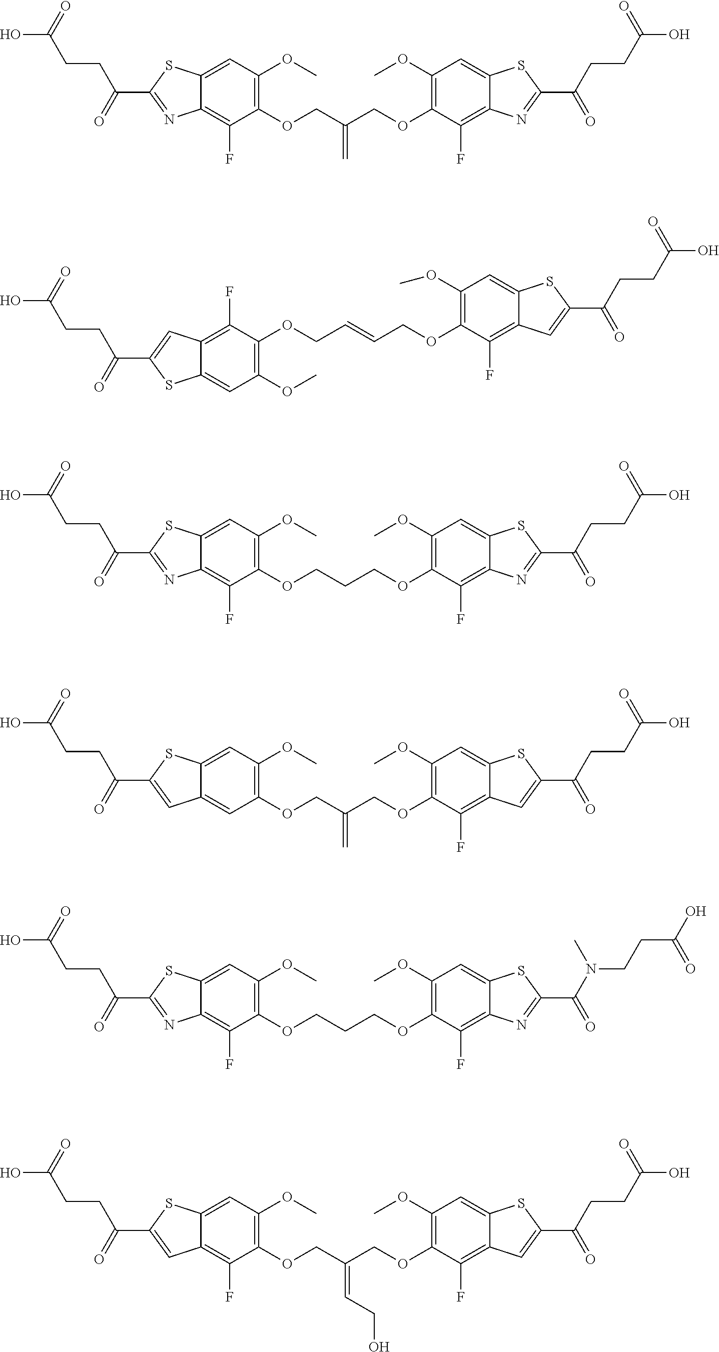 [SMILES: C=C(COc1c(OC)cc2sc(C(=O)CCC(=O)O)nc2c1F)COc1c(OC)cc2sc(C(=O)CCC(=O)O)nc2c1F ; COc1cc2sc(C(=O)CCC(=O)O)cc2c(F)c1OC/C=C/COc1c(OC)cc2sc(C(=O)CCC(=O)O)cc2c1F ; COc1cc2sc(C(=O)CCC(=O)O)nc2c(F)c1OCCCOc1c(OC)cc2sc(C(=O)CCC(=O)O)nc2c1F ; C=C(COc1cc2cc(C(=O)CCC(=O)O)sc2cc1OC)COc1c(OC)cc2sc(C(=O)CCC(=O)O)cc2c1F ; COc1cc2sc(C(=O)CCC(=O)O)nc2c(F)c1OCCCOc1c(OC)cc2sc(C(=O)N(C)CCC(=O)O)nc2c1F ; COc1cc2sc(C(=O)CCC(=O)O)cc2c(F)c1OCC(=CCO)COc1c(OC)cc2sc(C(=O)CCC(=O)O)cc2c1F]

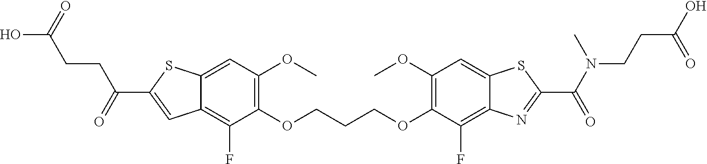 [SMILES: COc1cc2sc(C(=O)CCC(=O)O)cc2c(F)c1OCCCOc1c(OC)cc2sc(C(=O)N(C)CCC(=O)O)nc2c1F]

In some embodiments, the STING agonist moiety [D] is an isotopic derivative of any one of the compounds described in the two tables above and prodrugs and pharmaceutically acceptable salts thereof.

In some embodiments, the STING agonist moiety [D] is an isotopic derivative of any one of the compounds described in the two tables above and pharmaceutically acceptable salts thereof.

In some embodiments, the STING agonist moiety [D] is an isotopic derivative of any one of prodrugs of the compounds described in the two tables above and pharmaceutically acceptable salts thereof.

In some embodiments, the STING agonist moiety [D] is a deuterated compound.

In some embodiments, d5 is an integer ranging from about 2 to about 14, from about 2 to about 12, from about 2 to about 10, from about 2 to about 8, from about 2 to about 6, from about 2 to about 4, from about 4 to about 10, from about 4 to about 8, from about 4 to about 6, from about 6 to about 14, from about 6 to about 12, from about 6 to about 10, from about 6 to about 8, from about 8 to about 14, from about 8 to about 12, or from about 8 to about 10.

In some embodiments, d5 is an integer ranging from about 2 to about 8.

In some embodiments, d5 is 2, 4, 6, or 8. In some embodiments, d5 is 6 or 8.

In some embodiments, d5 is 8. In some embodiments, d5 is 6.

In some embodiments, the STING agonist moiety [D] is covalently bonded to the linker L¹, wherein L¹ may be cleavable or non-cleavable. The above defined provisos regarding the compound of formula (Y-1), (Y-2), (Y-3), (A), (B), (C), I, II, III, IV or V define functional groups, which preferably form the covalent bond to the linker L¹.

In some embodiments, a covalent bond between the compound of formula (Y-1), (Y-2), (Y-3), (A), (B), (C), I, II, III, IV or V and the linker L¹ is established by the reaction of a functional group of the compound of formula (Y-1), (Y-2), (Y-3), (A), (B), (C), I, II, III, IV or V with a functional group handle of the linker L¹; and wherein preferably the functional group of the compound of formula (Y-1), (Y-2), (Y-3), (A), (B), (C), I, II, III, IV or V is attached to or part of the substituents R₁, R₂, R₃, R⁴, X1, X₂, or X3 so that the linker L¹ will be covalently bonded to the compound of formula (Y-1), (Y-2), (Y-3), (A), (B), (C), I, II, III, IV or V.

It is intended that the definition of any substituent or variable at a particular location in a molecule be independent of its definitions elsewhere in that molecule. It is understood that substituents and substitution patterns on the compounds of formula (Y-1), (Y-2), (Y-3), (A), (B), (C), I, II, III, IV or V can be selected by one of ordinary skill in the art to provide compounds that are chemically stable and that can be readily synthesized by techniques known in the art as well as those methods set forth herein.

The present disclosure includes all stereoisomers of the compound and pharmaceutically acceptable salts thereof. Further, mixtures of stereoisomers as well as isolated specific stereoisomers are also included. During the course of the synthetic procedures used to prepare such compounds or in using racemization or epimerization procedures known to those skilled in the art, the products of such procedures can be a mixture of stereoisomers. The term “stereoisomer” as used herein refers to an isomer in which atoms or groups of atoms in the molecule are connected to each other in the same order but differ in spatial arrangement, including conformational isomers and conformational isomers. The configuration isomers include geometric isomers and optical isomers, and optical isomers mainly include enantiomers and diastereomers. The disclosure includes all possible stereoisomers of the compound.

Any of the compound of formula (Y-1), (Y-2), (Y-3), (A), (B), (C), I, II, III, IV or V is intended to include its isotopic derivative. The isotopic derivatives have structures depicted by the formulas given herein except that one or more atoms are replaced by an isotope. Examples of isotopes include and are not limited to isotopes of hydrogen, carbon, nitrogen, oxygen, phosphorous, fluorine, and chlorine, such as²H,³H,¹¹C,¹³C,¹⁴C,¹⁵N,¹⁷O,¹⁸O,¹⁸F,³⁵S,³⁶Cl, respectively.

In some embodiments, the isotopic derivative is a deuterated derivative, wherein one or more hydrogen atoms in one or more substituents are replaced with deuterium, e.g. all hydrogens in one or more alkyl substituents are replaced with deuterium (the respective moiety/moieties are then perdeuterated). Substitution with heavier isotopes, particularly deuterium (i.e., ²H or D), may afford certain therapeutic advantages resulting from greater metabolic stability, for example increased in vivo half-life or reduced dosage requirements or an improvement in therapeutic index. The concentration of such a heavier isotope, specifically deuterium, may be defined by the isotopic enrichment factor. The term “isotopic enrichment factor” as used herein means the ratio between the isotopic abundance and the natural abundance of a specified isotope.

In some embodiments, the linker L¹ is a linker comprising one or more cleavage elements, and each cleavage element is independently selected from a self-immolative spacer and a group that is susceptible to cleavage.

In some embodiments, the cleavage is selected from acid-induced cleavage, peptidase-induced cleavage, esterase-induced cleavage, glycosidase-induced cleavage, phosphodiesterase-induced cleavage, phosphatase-induced cleavage, protease-induced cleavage, lipase-induced cleavage, or disulfide bond cleavage.

In some embodiments, the linker L¹ comprises:
(i) a chain L^{c} of 2 to 100 atoms selected from carbon, nitrogen, oxygen, and sulfur atoms, which may be interrupted by 5- to 10-membered aryl and heteroaryl groups and/or 3- to 8-membered saturated carbocyclyl or heterocyclyl groups, wherein the aforementioned heteroaryl and heterocyclic groups comprise one or more, same or different heteroatoms selected from O, N or S, wherein said N- and/or S-atoms are independently oxidized or non-oxidized, and wherein each substitutable carbon or heteroatom in the chain is independently unsubstituted or substituted with one or more same or different substituents selected from deuterium, halogen, OH, ═O, C₁-C₆-alkyl, C₁-C₆-haloalkyl, C₃-C₆-cycloalkyl, C₃-C₆-halocycloalkyl, C₁-C₆-alkoxy, and C₁-C₆-haloalkoxy;
and preferably
a chain L^{C} of units selected from a linear or branched polyethylene glycol chain, a sequence of amino acids, and a linear or branched C₁-C₁₀-alkyl chain, wherein each substitutable carbon or heteroatom of the aforementioned units may be unsubstituted or substituted with one or more, same or different substituents selected from deuterium, halogen, OH, ═O, C₁-C₆-alkyl, C₁-C₆-haloalkyl, C₃-C₆-cycloalkyl, C₃-C₆-halocycloalkyl, C₁-C₆-alkoxy, and C₁-C₆-haloalkoxy;
(ii) a functional group handle H^{1A}, which is covalently bonded to the compound of formula (Y-1), (Y-2), (Y-3), (A), (B), (C), I, II, III, IV or V;
(iii) a functional group handle H^{1B} suitable for forming a covalent bond to a targeting moiety T.

In some embodiments, the linker L¹ has the structure H^{1A}-L^{C}-H^{1B}

In some embodiments, the compound-linker construct is of formula S2a:

D-H^{1A}-L^{C}-H^{1B} (S2a)
wherein H^{1B} is a monovalent linker moiety comprising a functional group capable of forming a covalent bond to a targeting moiety T;
D is the same as defined herein.

In some embodiments of the conjugates of the present disclosure, H^{1B} is selected from the group consisting of

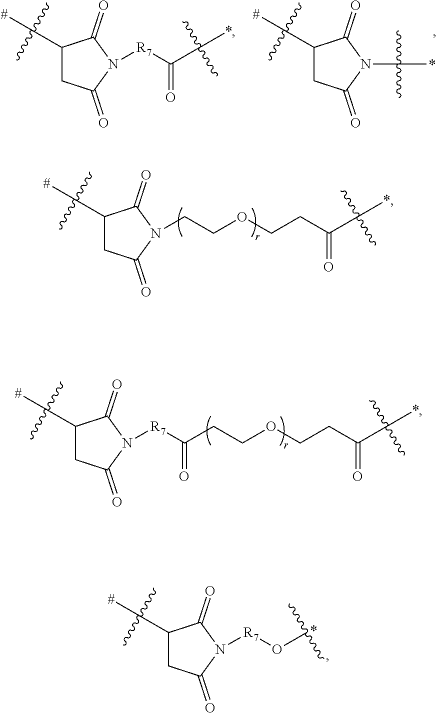

wherein:

R₇ is —O—, —NR^{7a}, —(C₁-C₁₀ alkyl)-, —(C₁-C₁₀ alkenyl)-, —(C₁-C₁₀ alkynyl)-, —(C₃-C₅ cycloalkyl)-, -aryl-, —O—(C₁-C₅ alkyl)-, —O—(C₁-C₁₀ alkenyl)-, —O—(C₁-C₁₀ alkynyl)-, —(C₁-C₁₀ alkyl)-(C₃-C₅ cycloalkyl)-, —(C₁-C₁₀ alkyl)-aryl-, —(C₂-C₁₀ alkenyl)-(C₃-C₅ cycloalkyl)-, —(C₂-C₁₀ alkenyl)-aryl-, —(C₂-C₁₀ alkynyl)-(C₃-C₅ cycloalkyl)-, —(C₂-C₁₀ alkynyl)-aryl-, —(C₃-C₅ cycloalkyl)-(C₁-C₁₀alky)-, -aryl-(C₁-C₁₀alky)-, —(C₃-C₅cycloalkyl)-(C₂-C₁₀ alkenyl)-, -aryl-(C₂-C₁₀ alkenyl)-, —(C₃-C₅ cycloalkyl)-(C₂-C₁₀ alkynyl)-, -aryl-(C₂-C₁₀ alkynyl)-, -(3- to 8-membered heterocycloalkyl)-, -(5- to 8-membered heteroaryl)-, —(C₁-C₁₀ alkyl)-(3- to 8-membered heterocycloalkyl)-, —(C₁-C₁₀ alkyl)-(5- to 8-membered heteroaryl)-, —(C₂-C₁₀ alkenyl)-(3- to 8-membered heterocycloalkyl)-, —(C₂-C₁₀ alkenyl)-(5- to 8-membered heteroaryl)-, —(C₂-C₁₀ alkynyl)-(3- to 8-membered heterocycloalkyl)-, —(C₂-C₁₀ alkynyl)-(5- to 8-membered heteroaryl)-, -(3- to 8-membered heterocycloalkyl)-(C₁-C₁₀ alkyl)-, -(5- to 8-membered heteroaryl)-(C₁-C₁₀ alkyl)-, -(3- to 8-membered heterocycloalkyl)-(C₂-C₁₀ alkenyl)-, -(5- to 8-membered heteroaryl)-(C₂-C₁₀ alkenyl)-, -(5- to 8-membered heteroaryl)-(C₂-C₁₀ alknyl)-, -(5- to 8-membered heteroaryl)-(C₂-C₁₀ alknyl)-, —O—C(O)—(CH₂CH₂O)r-(CH₂)₂—, —(CH₂CH₂O)r-, —(CH₂CH₂O)r (CH₂)₂— or —CH(CH₂NH₂)—, wherein the alkyl, alkenyl, alkynyl, cycloalkyl, aryl, heterocycloalkyl, or heteroaryl is optionally substituted;
R^{7a} is H, hydroxy, or C₁₋₄ alkyl;
each r is independently an integer ranging from about 1 to about 12;
t is an integer ranging from about 1 to about 8; and
# denotes attachment to T and * denotes attachment to L^{C}.

In some embodiments, R₇ is —(C₁-C₁₀ alkyl)-, such as —(C₁-C₆ alkyl)-.

In some embodiments of the conjugates of the present disclosure, H^{1B} is selected from the group consisting of

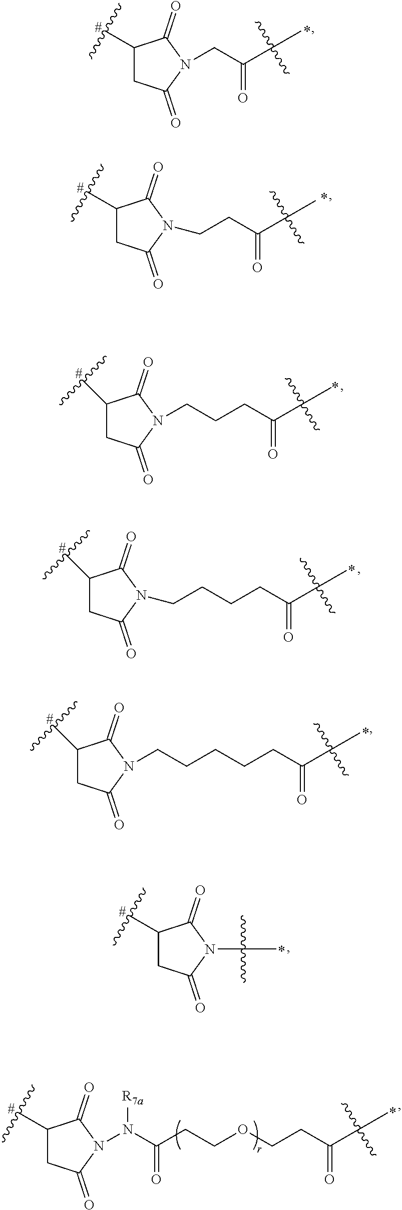

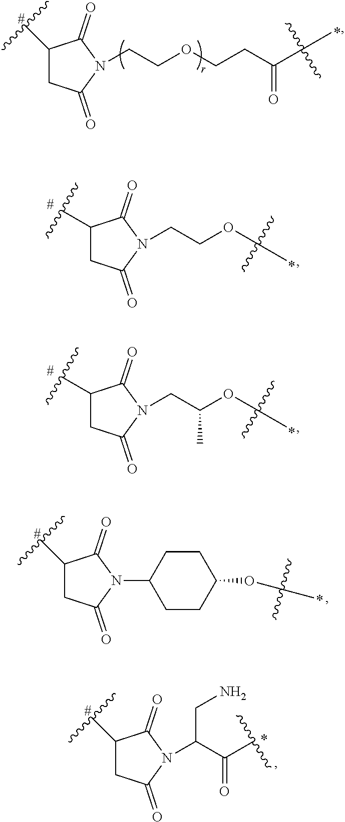

wherein:
r is an integer ranging from about 4 to about 6; and
# denotes attachment to T and * denotes attachment to L^{C}.

In some embodiments of the compound-linker constructs of the present disclosure, H^{1B} is selected from the group consisting of

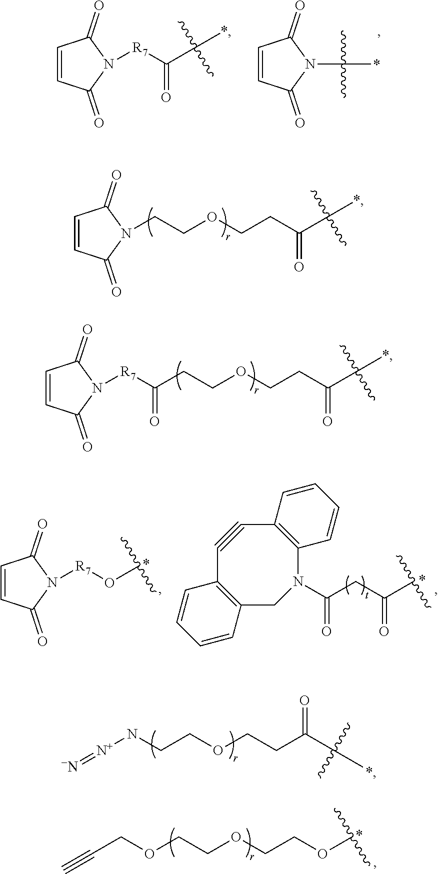

wherein:
R₇ is —O—, —NR^{7a}, —(C₁-C₁₀ alkyl)-, —(C₁-C₁₀ alkenyl)-, —(C₁-C₁₀ alkynyl)-, —(C₃-C₅ cycloalkyl)-, -aryl-, —O—(C₁—C₈ alkyl)-, —O—(C₁-C₁₀ alkenyl)-, —O—(C₁-C₁₀ alkynyl)-, —(C₁-C₁₀ alkyl)-(C₃-C₅ cycloalkyl)-, —(C₁-C₁₀ alkyl)-aryl-, —(C₂-C10 alkenyl)-(C₃-C₅ cycloalkyl)-, —(C₂-C₁₀ alkenyl)-aryl-, —(C₂-C₁₀ alkynyl)-(C₃-C₅ cycloalkyl)-, —(C₂-C₁₀ alkynyl)-aryl-, —(C₃-C₅ cycloalkyl)-(C₁-C₁₀alky)-, -aryl-(C₁-C₁₀alky)-, —(C₃-C₅cycloalkyl)-(C₂-C₁₀ alkenyl)-, -aryl-(C₂-C₁₀ alkenyl)-, —(C₃-C₅ cycloalkyl)-(C₂-C₁₀ alkynyl)-, -aryl-(C₂-C₁₀ alkynyl)-, -(3- to 8-membered heterocycloalkyl)-, -(5- to 8-membered heteroaryl)-, —(C₁-C₁₀ alkyl)-(3- to 8-membered heterocycloalkyl)-, —(C₁-C₁₀ alkyl)-(5- to 8-membered heteroaryl)-, —(C₂-C₁₀ alkenyl)-(3- to 8-membered heterocycloalkyl)-, —(C₂-C₁₀ alkenyl)-(5- to 8-membered heteroaryl)-, —(C₂-C₁₀ alkynyl)-(3- to 8-membered heterocycloalkyl)-, —(C₂-C₁₀ alkynyl)-(5- to 8-membered heteroaryl)-, -(3- to 8-membered heterocycloalkyl)-(C₁-C₁₀ alkyl)-, -(5- to 8-membered heteroaryl)-(C₁-C₁₀ alkyl)-, -(3- to 8-membered heterocycloalkyl)-(C₂-C₁₀ alkenyl)-, -(5- to 8-membered heteroaryl)-(C₂-C₁₀ alkenyl)-, -(5- to 8-membered heteroaryl)-(C₂-C₁₀ alknyl)-, -(5- to 8-membered heteroaryl)-(C₂-C₁₀ alknyl)-, —O—C(O)—(CH₂CH₂O)ᵣ—(CH₂)₂—, —(CH₂CH₂O)ᵣ—, —(CH₂CH₂O)ᵣ (CH₂)₂— or —CH(CH₂NH₂)—, wherein the alkyl, alkenyl, alkynyl, cycloalkyl, aryl, heterocycloalkyl, or heteroaryl is optionally substituted;
R⁷, is H, hydroxy, or C₁₋₄ alkyl;
each r is independently an integer ranging from about 1 to about 12;
t is an integer ranging from about 1 to about 8; and
# denotes attachment to T and * denotes attachment to L^{C}.

In some embodiments, R₇ is —(C₁-C₁₀ alkyl)-, such as —(C₁-C₆ alkyl)-.

In some embodiments of the compound-linker constructs of the present disclosure, H^{1B} is selected from the group consisting of

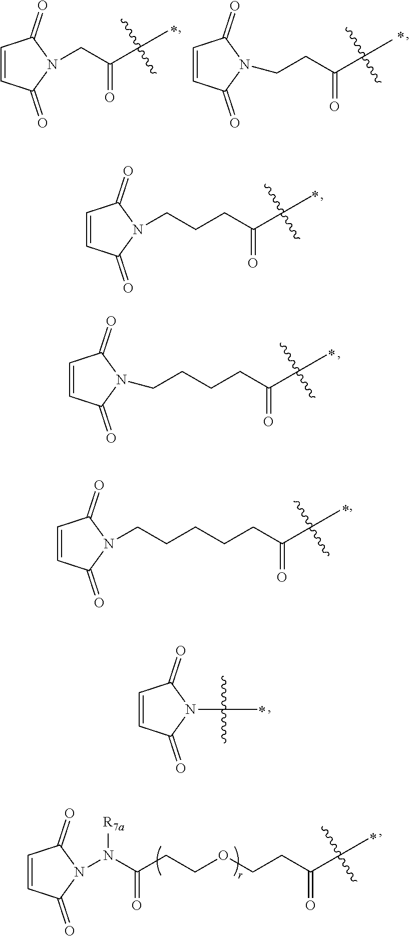

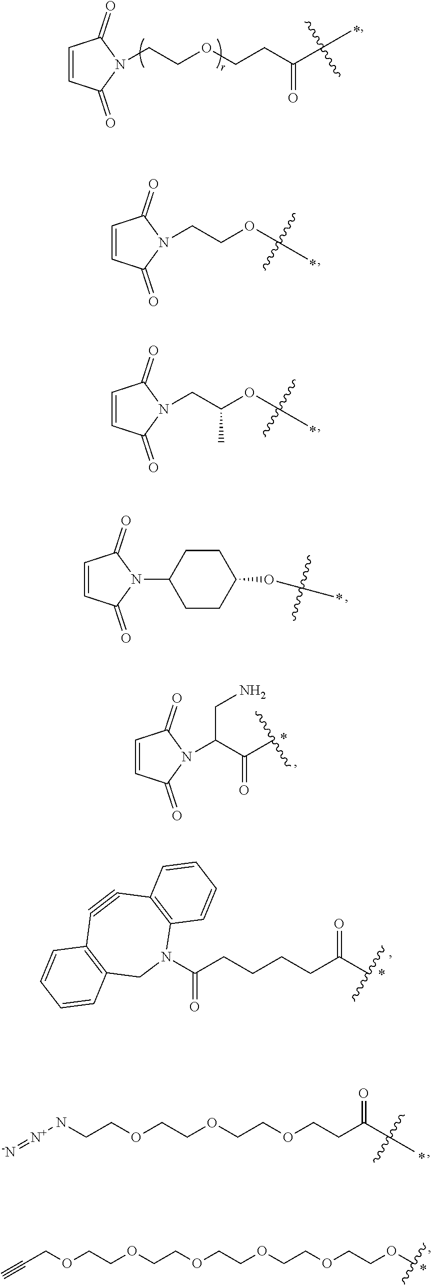

wherein:
r is an integer ranging from about 4 to about 6; and
* denotes attachment to L^{C}.

In some embodiments, L^{C} is,

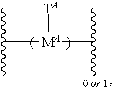

wherein
M^{A} when present, is an amino acid residue or a peptide moiety comprising at least two amino acids;
T^{A} when present, is a hydrophilic group.

In some embodiments, H^{1A} is a divalent linker moiety connecting D to M^{A}, when M^{A} is present, or to H^{1B} when M^{A} is absent.

In some embodiments, H^{1A}comprises at least one cleavable bond such that when the bond is cleaved, D is released in an active form for its intended therapeutic effect.

In some embodiments, H^{1A} comprises one cleavable bond. In some embodiments, H^{1A} comprises multiple cleavage sites or bonds.

In some embodiments, the structure and sequence of the cleavable bond in H^{1A} can be such that the bond is cleaved by the action of enzymes present at the target site. In some embodiments, the cleavable bond can be cleavable by other mechanisms.

In some embodiments, the cleavable bond(s) can be enzymatically cleaved by one or more enzymes, including a tumor-associated protease, to liberate the Drug unit or D, wherein the conjugate of the present disclosure, or intermediate, or scaffold thereof, is protonated in vivo upon release to provide a Drug unit or D.

In some embodiments, H^{1A} is

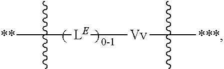

wherein L^{E}, when present, is —NH—[(CH₂CH₂O)p-(CH₂)₀₋₂]q-, —NH—(C₁-C₆alkyl)-, or —NH—[(CH₂CH₂O)p-(CH₂)₀₋₂]q-C(O)—NH—(C₁-C₆alkyl)-, wherein p is an integer ranging from about 1 to about 20, and q is an integer ranging from about 1 to about 10;
each V independently is a natural or unnatural amino acid, diamine or amino alcohol unit;
v is an integer ranging from about 0 to about 12;
** denotes attachment to M^{A}, when M^{A} is present, or to H^{1B}, when M^{A} is absent;
*** denotes attachment to D.

In some embodiments, H^{1A} is L^{E}.

In some embodiments, H^{1A} is Vv.

In some embodiments, L^{E} comprises at least one PEG unit.

In some embodiments, the PEG unit comprises at least 1 subunit, at least 2 subunits, at least 3 subunits, at least 4 subunits, at least 5 subunits, or at least 6 subunits. In some embodiments, the PEG unit comprises at least 4 subunits, at least 3 subunits, at least 2 subunits, or at least 1 subunit. In some embodiments, the PEG unit comprises at least 1 subunit. In some embodiments, the PEG unit comprises at least 2 subunits.

In some embodiments, p is an integer ranging from about 1 to about 15, from about 1 to about 10, from about 1 to about 9, from about 1 to about 8, from about 1 to about 7, from about 1 to about 6, or from about 1 to about 5.

In some embodiments, p is an integer ranging from about 1 to about 6. In some embodiments, p is an integer ranging from about 1 to about 4. In some embodiments, p is an integer ranging from about 1 to about 2. Preferably is 2.

In some embodiments, q is an integer ranging from about 1 to about 15, from about 1 to about 10, from about 1 to about 9, from about 1 to about 8, from about 1 to about 7, from about 1 to about 6, or from about 1 to about 5.

In some embodiments, q is 1, 2, 3, 4, or 5. In some embodiments, q is 2.

In some embodiments, v is an integer ranging from about 1 to about 12 (e.g., 1 to 6, or 1 to 4, or 1 to 3, or 1, 2, 3, 4, 5, 6, 7, 8, 9, 10, 11, or 12).

In some embodiments, v is 0, 1, 2, 3, 4, or 5.

In some embodiments, each V independently is a diamine or amino alcohol and/or a D or L isomer. In some embodiments, each V independently is a diamine. Diamine refers to an amino compound containing two amino groups, such as ethylene diamine, propylene diamine, hexamethylene diamine, p-phenylenediamine, etc. In some embodiments, each V independently is an amino alcohol. Amino alcohol refers to a substance having an amino group and an alcoholic hydroxyl group in the same molecule of an organic compound. For example, methanolamine, 2-hydroxyethylamine, S-1-amino-2-propanol, p-aminophenylethanol, N-(tert-butoxycarbonyl)ethanolamine and the like.

In some embodiments, each V independently is a natural or unnatural amino acid and/or a D or L isomer. In some embodiments, each V independently is an alpha, beta, or gamma amino acid that is natural or non-natural. In some embodiments, at least one V is a natural amino acid. In some embodiments, at least one V is a non-natural amino acid.

In some embodiments, Vv does not comprise natural amino acids. In some embodiments, Vv does not comprise non-natural amino acids. In some embodiments, Vv comprises a natural amino acid linked to a non-natural amino acid. In some embodiments, Vv comprises a natural amino acid linked to a D-isomer of a natural amino acid.

In some embodiments, Vv is a dipeptide, e.g., -Val-Cit-, -Phe-Lys-, -Val-Ala- or Glu-Ala. In some embodiments, Vv is a monopeptide, a dipeptide, a tripeptide, a tetrapeptide, a pentapeptide, a hexapeptide, a heptapeptide, an octapeptide, a nonapeptide, a decapeptide, an undecapeptide, or a dodecapeptide unit.

In some embodiments, Vv is a peptide (e.g., a peptide of 1 to 12 amino acids), which is conjugated directly to D. In some embodiments, the peptide is a single amino acid. In some embodiments, the peptide is a dipeptide. In some embodiments, the peptide is a tripeptide.

In some embodiments, each amino acid in Vv is independently selected from alanine, P-alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, selenocysteine, ornithine, penicillamine, aminoalkanoic acid, aminoalkynoic acid, aminoalkanedioic acid, aminobenzoic acid, amino-heterocyclo-alkanoic acid, heterocyclo-carboxylic acid, citrulline, statine, diaminoalkanoic acid, and derivatives thereof.

In some embodiments, each amino acid in Vv is independently selected from alanine, P-alanine, arginine, aspartic acid, asparagine, histidine, glycine, glutamic acid, glutamine, phenylalanine, lysine, leucine, serine, tyrosine, threonine, isoleucine, proline, tryptophan, valine, citrulline, and derivatives thereof.

In some embodiments, each amino acid in Vv is independently selected from the proteinogenic and the non-proteinogenic amino acids.

In some embodiments, each amino acid in Vv is independently selected from L or D isomers of the following amino acids: alanine, P-alanine, arginine, aspartic acid, asparagine, cysteine, histidine, glycine, glutamic acid, glutamine, phenylalanine, lysine, leucine, methionine, serine, tyrosine, threonine, tryptophan, proline, ornithine, penicillamine, aminoalkynoic acid, aminoalkanedioic acid, heterocyclo- carboxylic acid, citrulline, statine, diaminoalkanoic acid, valine, citrulline, and derivatives thereof.

In some embodiments, each amino acid in Vv is independently cysteine, homocysteine, penicillamine, ornithine, lysine, serine, threonine, glycine, glutamine, alanine, aspartic acid, glutamic acid, selenocysteine, proline, glycine, isoleucine, leucine, methionine, valine, citrulline, or alanine.

In some embodiments, each amino acid in Vv is independently selected from L-isomers of the following amino acids: alanine, P-alanine, arginine, aspartic acid, asparagine, histidine, glycine, glutamic acid, glutamine, phenylalanine, lysine, leucine, serine, tyrosine, threonine, isoleucine, tryptophan, citrulline, and valine.

In some embodiments, each amino acid in Vv is independently selected from D-isomers of the following amino acids: alanine, B-alanine, arginine, aspartic acid, asparagine, histidine, glycine, glutamic acid, glutamine, phenylalanine, lysine, leucine, serine, tyrosine, threonine, isoleucine, tryptophan, citrulline, and valine.

In some embodiments, each amino acid in Vv is alanine, P-alanine, glycine, glutamic acid, isoglutamic acid, isoaspartic acid, valine citrulline, or aspartic acid.

In some embodiments, Vv comprises P-alanine. In some embodiments, W comprises (P-alanine)-(alanine).

In some embodiments, Vv comprises (P-alanine) and optionally glutamic acid, isoglutamic acid, aspartic acid, isoaspartic acid, valine, (valine)-(alanine), (alanine)-(alanine), or (valine)-(citruline).

In some embodiments, Vv comprises (glutamic acid)-(alanine).

In some embodiments, Vv comprises glutamic acid and optionally alanine, glycine, isoglutamic acid, aspartic acid, isoaspartic acid, valine, (valine)-(alanine), (alanine)-(alanine), or (valine)-(citruline).

In some embodiments, Vv comprises 2,3-diaminopropanoic acid. In some embodiments, Vv comprises (R)-2,3-diaminopropanoic acid. In some embodiments, Vv comprises glutamic acid. In some embodiments, Vv comprises (glutamic acid)-(alanine). In some embodiments, Vv comprises (glutamic acid)-(glycine)-(alanine).

In some embodiments, Vv comprises L-glutamic acid, D-glutamic acid, (L-glutamic acid)-(L-alanine), (L-glutamic acid)-(D-alanine), (D-glutamic acid)-(L-alanine), (D-glutamic acid)-(D-alanine),, (L-glutamic acid)-(glycine)-(L-alanine), D-glutamic acid)-(glycine)-(D-alanine), (L-glutamic acid)-(glycine)-(D-alanine), or (D-glutamic acid)-(glycine)-(L-alanine).

In some embodiments, Vv comprises a carbamate bond in addition to one or more amino acids.

In some embodiments, H^{1A} (e.g., Vv) is selective for enzymatic cleavage (e.g., by a particular enzyme). In some embodiments, the particular enzyme is a tumor-associated protease.

In some embodiments, H^{1A} (e.g., Vv) comprises a bond whose cleavage is catalyzed by cathepsin B, cathepsin C, cathepsin D, or a plasmin protease.

In some embodiments, H^{1A} comprises a sugar cleavage site.

In some embodiments, H^{1A} comprises a sugar moiety (Su) linked via an oxygen glycosidic bond to a self-immolative group.

In some embodiments, a “self-immolative group” can be a tri-functional chemical moiety that is capable of covalently linking together three spaced chemical moieties (i.e., the sugar moiety (via a glycosidic bond), a drug unit (directly or indirectly), and M^{A} (directly or indirectly) when M^{A} is present or H^{1B} when M^{A} is absent.

In some embodiments, the glycosidic bond can be cleaved at the target site to initiate a self-immolative reaction sequence that leads to a release of the drug.

In some embodiments, L^{E}, when present, is —NH—(CH₂CH₂O)₁₋₄—(CH₂)₂—. In some embodiments, L^{E}, when present, is —NH—(CH₂CH₂O)₂—(CH₂)₂—. In some embodiments, L^{E}, when present, is —NH—(CH₂CH₂O)₃—(CH₂)O₂—.

In some embodiments, L^{E}, when present, is —NH—(CH₂CH₂O)₃—(CH₂)₁—. In some embodiments, L^{E}, when present, is —NH—(CH₂CH₂O)₃—(CH₂)₂—. In some embodiments, L^{E}, when present, is —NH—CH₂CH₂O—(CH₂)O₂—.

In some embodiments, L^{E}, when present, is —NH—CH₂CH₂O—. In some embodiments, L^{E}, when present, is —NH—(C₁-C₆ alkyl)-. In some embodiments, L^{E}, when present, is —NH—CH₂—CH(CH₃)—. In some embodiments, L^{E}, when present, is —NH—[(CH₂CH₂O)₁₄—(CH₂)₂—C(O)—NH—(C₁-C₆ alkyl)-. In some embodiments, L^{E}, when present, is —NH—CH₂CH₂O—(CH₂)₂—C(O)—NH—(CH₂)₂—.

In some embodiments, L^{E} is —NH—(CH₂CH₂O)₂—(CH₂)₂—, —NH—CH₂—CH(CH₃)—, or —NH—[(CH₂CH₂O)₁₋₄—(CH₂)₂—C(O)—NH—(CH₂)₂—.

In some embodiments, H^{1A} is selected from the group consisting of:

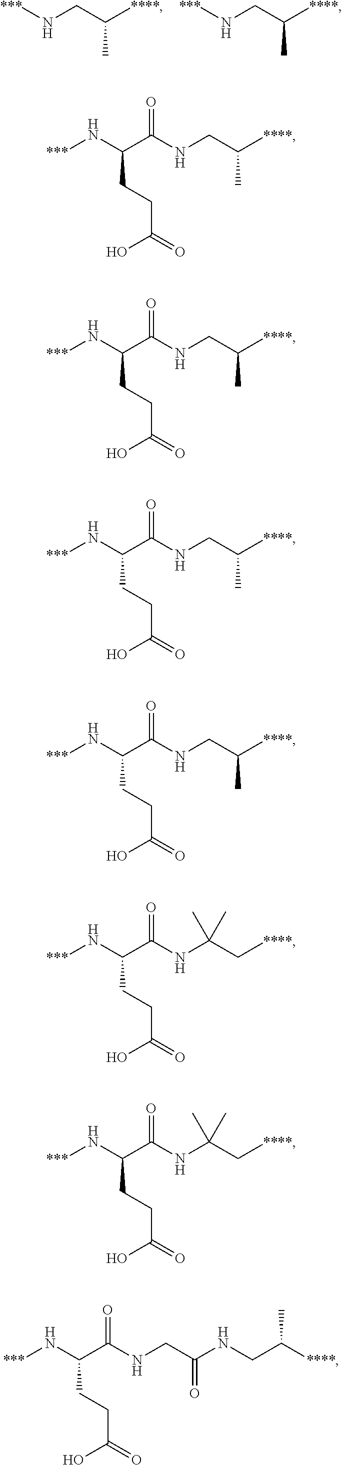

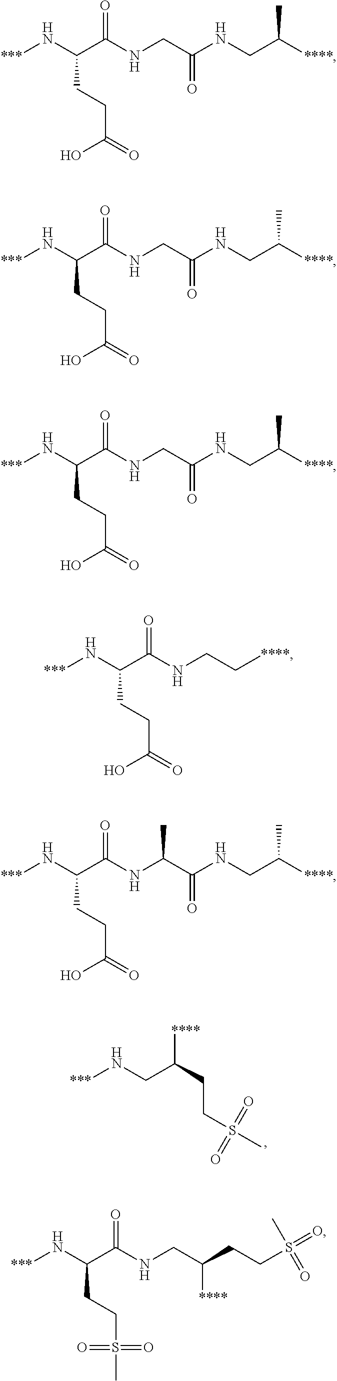

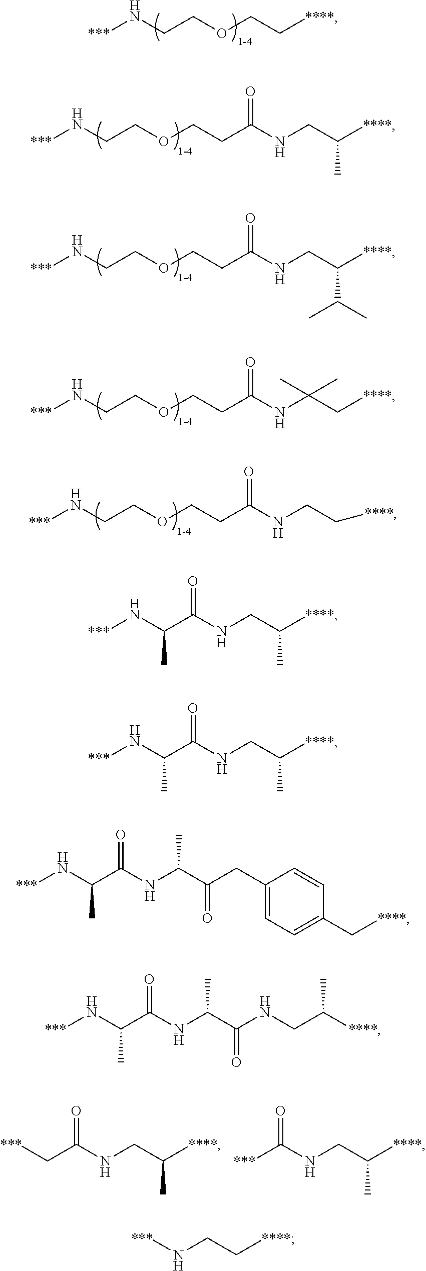

wherein *** denotes attachment to M^{A} when is present, or to H^{1B} when is absent; and ****denotes attachment to D.

In some embodiments, H^{1A} is selected from the group consisting of:

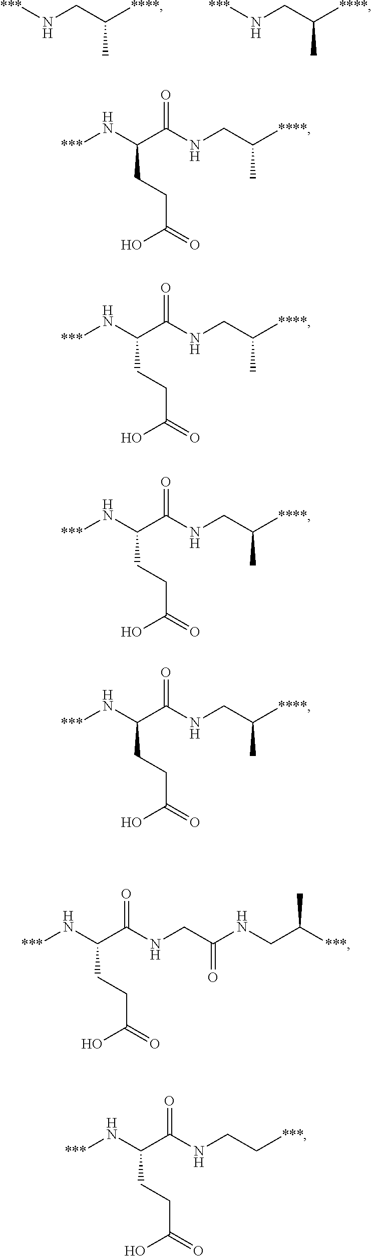

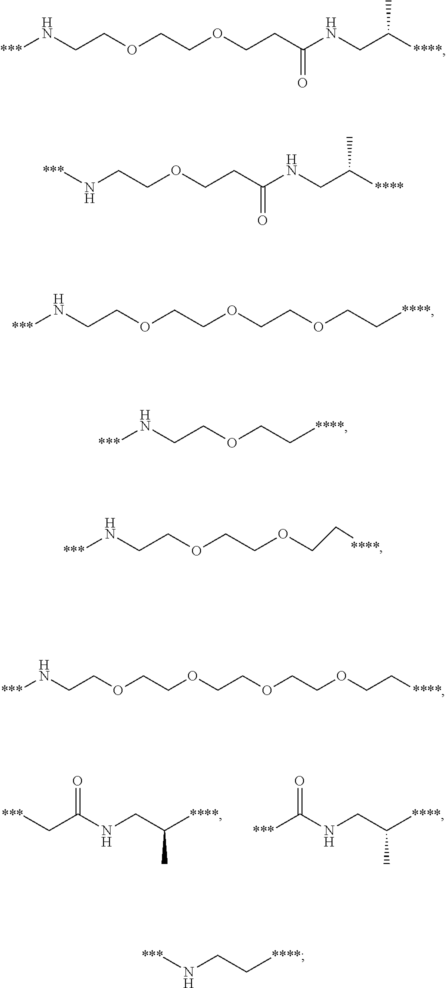

wherein *** denotes attachment to M^{A} when is present, or to H^{1B} when is absent; and ****denotes attachment to D.

In some embodiments, each H^{1A}, when present, independently is:

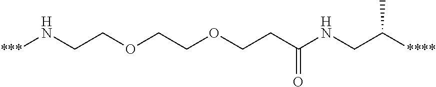

wherein *** denotes attachment to M^{A} when is present, or to H^{1B} when is absent; and ****denotes attachment to D.

In some embodiments, each H^{1A}, when present, independently is:

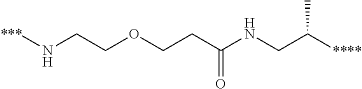

wherein *** denotes attachment to M^{A} when is present, or to H^{1B} when is absent; and ****denotes attachment to D.

In some embodiments, each H^{1A}, when present, independently is:

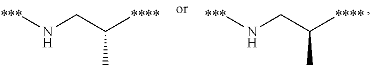

wherein *** denotes attachment to M^{A} when is present, or to H^{1B} when is absent; and ****denotes attachment to D.

In some embodiments, H^{1A} is selected from the group consisting of:

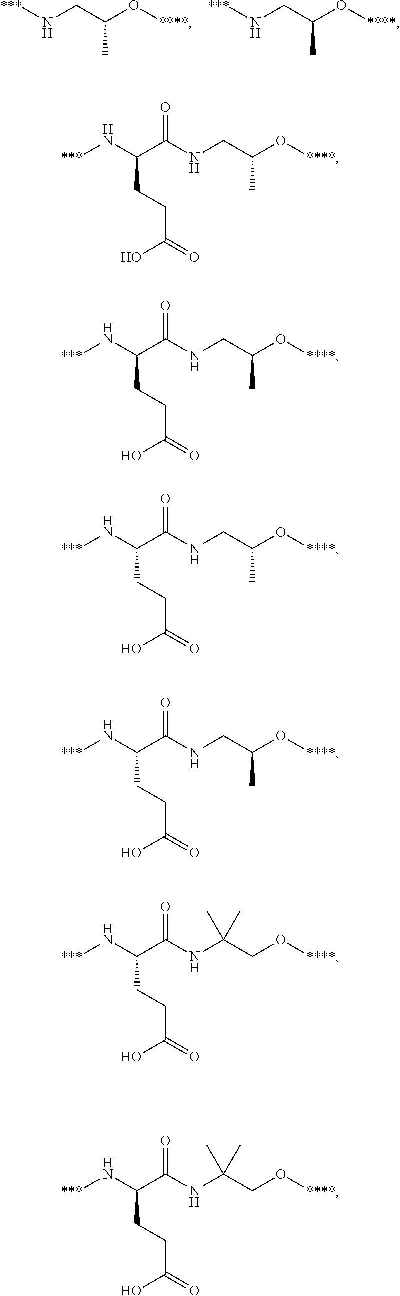

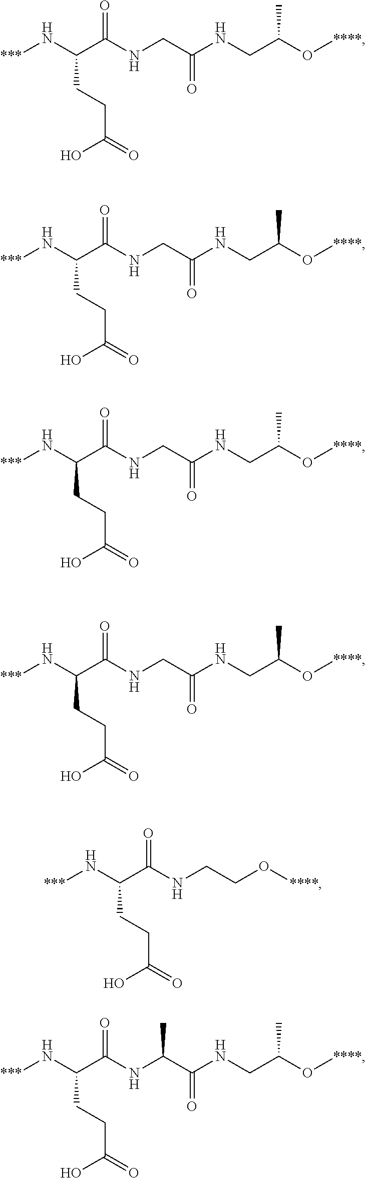

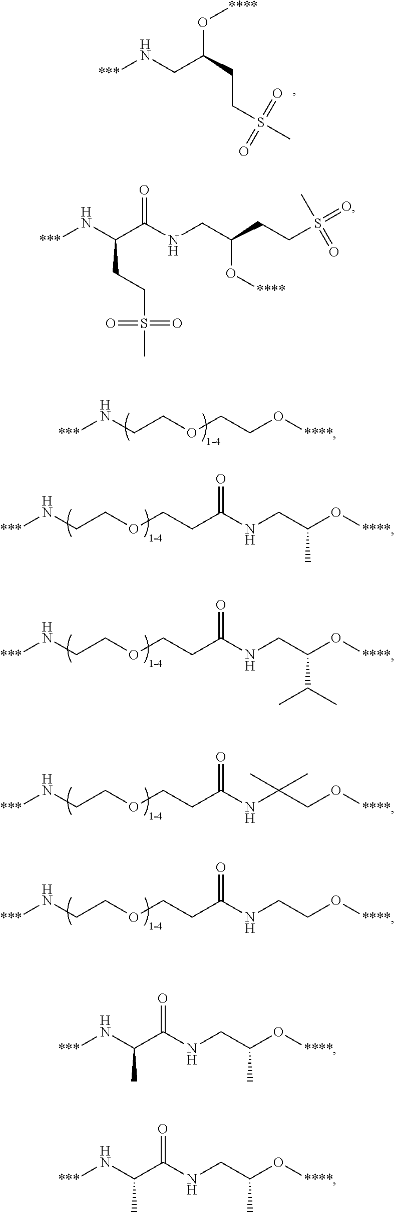

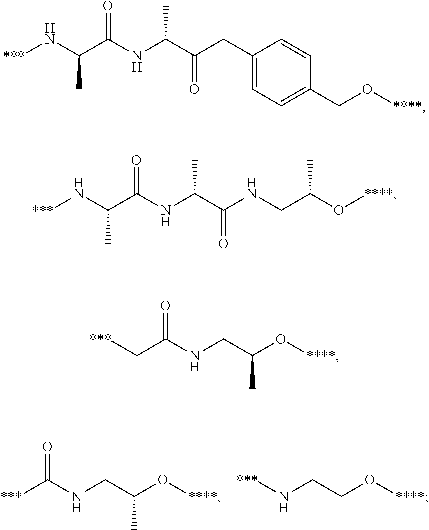

wherein *** denotes attachment to M^{A} when is present, or to H^{1B} when is absent; and ****denotes attachment to D.

In some embodiments, H^{1A} is selected from the group consisting of:

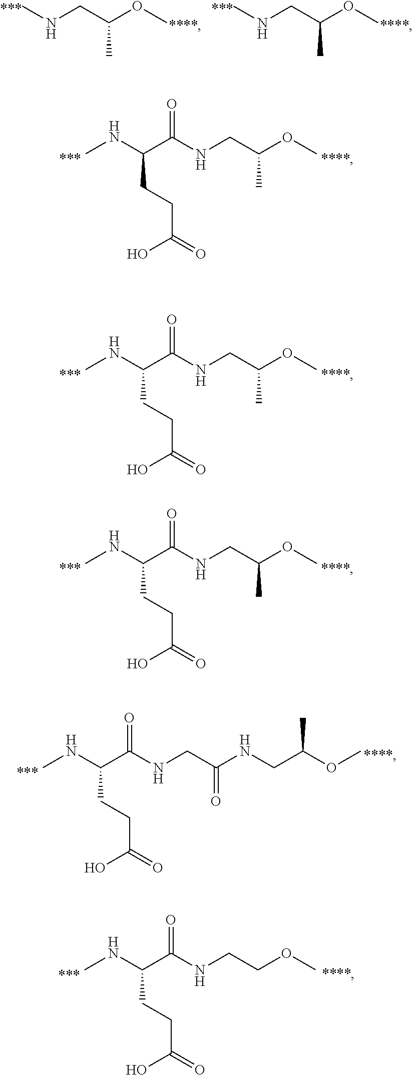

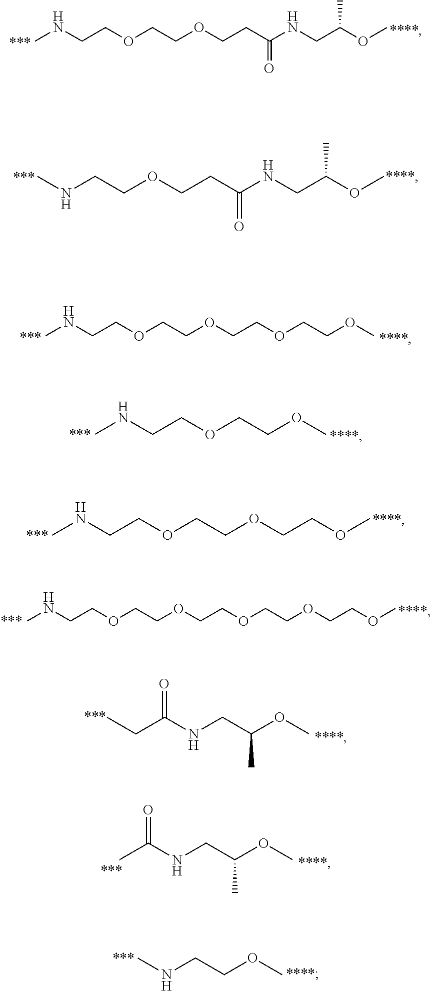

wherein *** denotes attachment to M^{A} when is present, or to H^{1B} when is absent; and ****denotes attachment to D.

In some embodiments, each H^{1A}, when present, independently is:

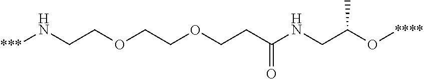

wherein *** denotes attachment to M^{A} when is present, or to H^{1B} when is absent; and ****denotes attachment to D.

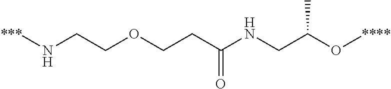

In some embodiments, each H^{1A}, when present, independently is:

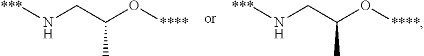

wherein *** denotes attachment to M^{A} when is present, or to H^{1B} when is absent; and ****denotes attachment to D.

In some embodiments, each H^{1A}, when present, independently is: H - or wherein *** denotes attachment to M^{A} when is present, or to H^{1B} when is absent; and ****denotes attachment to D.

In some embodiments, M^{A} comprises one amino acid residue.

In some embodiments, M^{A} comprises one glutamic acid residue.

In some embodiments, M^{A} comprises a peptide moiety of at least two amino acids.

In some embodiments, amino acid is referred to herein as “AA” and amino acids as “AAs”.

In some embodiments, M^{A} is a moiety that is capable of forming a covalent bond with a —H^{1A}-D unit and allows for the attachment of multiple drugs.

In some embodiments, M^{A} comprises a single AA unit or has two or more AA units (e.g., from 2 to 10, from 2 to 6, or 2, 3, 4, 5 or 6) wherein the AA units are each independently a natural or non-natural amino acid, an amino alcohol, an amino aldehyde, a diamine, a polyamine, or combinations thereof.

In some embodiments, in order to have the requisite number of attachments, at least one of the AA units will have a functionalized side chain to provide for attachment of the —H^{1A}-D unit. In some embodiments, exemplary functionalized AA units (e.g., amino acids, amino alcohols, or amino aldehydes) include, for example, azido or alkyne functionalized AA units (e.g., amino acid, amino alcohol, or amino aldehyde modified to have an azide group or alkyne group).

In some embodiments, M^{A} comprises 2 to 12 AA units. In some embodiments, M^{A} comprises 2 to 10 AA units. In some embodiments, M^{A} comprises 2 to 6 AA units. In some embodiments, MA comprises 2, 3, 4, 5 or 6 AA units.

In some embodiments, M^{A} has 2 AA units. In some embodiments, the peptide moiety has 3 AA units. In some embodiments, the peptide moiety has 4 AA units. In some embodiments, the peptide moiety has 5 AA units. In some embodiments, the peptide moiety has 6 AA units.

In some embodiments, attachment within M^{A} or with the other components of the conjugate, intermediate thereof, or scaffold, can be, for example, via amino, carboxy, or other functionalities.

In some embodiments, each amino acid in M^{A} can be independently D or L isomer of a thiol containing amino acid. In some embodiments, each amino acid in MA can be independently a D isomer of a thiol containing amino acid. In some embodiments, each amino acid in MA can be independently an L isomer of a thiol containing amino acid. In some embodiments, the thiol containing amino acid can be, for example, cysteine, homocysteine, or penicillamine.

In some embodiments, each amino acid in M^{A} can be independently the L or D isomer of the following amino acids: alanine (including P-alanine), arginine, aspartic acid, asparagine, cysteine, histidine, glycine, glutamic acid, glutamine, phenylalanine, lysine, leucine, methionine, serine, tyrosine, threonine, tryptophan, proline, ornithine, penicillamine, aminoalkynoic acid, aminoalkanedioic acid, heterocyclo-carboxylic acid, citrulline, statine, diaminoalkanoic acid, stereoisomers thereof, or derivatives thereof.

In some embodiments, each amino acid in M^{A} is independently cysteine, homocysteine, penicillamine, ornithine, lysine, serine, threonine, glycine, glutamine, alanine, aspartic acid, glutamic acid, selenocysteine, proline, glycine, isoleucine, leucine, methionine, valine, alanine, or a stereoisomer thereof.

In some embodiments, M^{A} comprises a monopeptide, a dipeptide, tripeptide, tetrapeptide, or pentapeptide.

In some embodiments, M^{A} comprises a pentapeptide.

In some embodiments, M^{A} comprises at least about five amino acids (e.g., 5, 6, 7, 8, 9, or 10 amino acids).

In some embodiments, M^{A} comprises at most about ten amino acids.

In some embodiments, each amino acid in M^{A} independently is glycine, serine, glutamic acid, lysine, aspartic acid, and cysteine.

In some embodiments, M^{A} comprises at least four glycines and at least one glutamic acid e.g., (glycine)4 and glutamic acid, wherein the glutamic acid is at any position along the peptide chain, such as, for example, (glutamic acid)-(glycine)4; (glycine)-(glutamic acid)-(glycine)3; (glycine)2-(glutamic acid)-(glycine)2; (glycine)3-(glutamic acid)-(glycine); or (glycine)4-(glutamic acid).

In some embodiments, M^{A} comprises (glycine)4-(glutamic acid). In some embodiments, the peptide moiety comprises (glutamic acid)-(glycine)4.

In some embodiments, M^{A} comprises at least four glycines and at least one serine, e.g., (glycine)4 and serine wherein the serine is at any position along the peptide chain, such as, for example, (serine)-(glycine)4; (glycine)-(serine)-(glycine)3; (glycine)2-(serine)-(glycine)2; (glycine)3-(serine)-(glycine); or (glycine)4-(serine).

In some embodiments, M^{A} comprises (glycine)4-(serine). In some embodiments, the peptide moiety comprises (serine)-(glycine)4.

In some embodiments, M^{A} comprises (β-alanine)-(glycine)4-(serine) wherein the serine is at any position along the peptide chain, such as, for example, (β-alanine)-(serine)-(glycine)4; (β-alanine)-(glycine)-(serine)-(glycine)3; (B-alanine)-(glycine)2-(serine)-(glycine)2; (B-alanine)-(glycine)3-(serine)-(glycine); or (B-alanine)-(glycine)4-(serine).

In some embodiments, M^{A} comprises (glycine)4-(serine)-(glutamic acid) wherein the serine is at any position along the peptide chain, such as, for example, (serine)-(glycine)4-(glutamic acid); (glycine)-(serine)-(glycine)3-(glutamic acid); (glycine)2-(serine)-(glycine)2-(glutamic acid); (glycine)3-(serine)-(glycine)-(glutamic acid); or (glycine)4-(serine)-(glutamic acid).

In some embodiments, the peptide moiety comprises (β-alanine)-(glycine)4-(serine)-(glutamic acid) wherein the serine is at any position along the peptide chain, such as, for example, (β-alanine)-(serine)-(glycine)4-(glutamic acid); (β-alanine)-(glycine)-(serine)-(glycine)3-(glutamic acid); (β-alanine)-(glycine)2-(serine)-(glycine)2-(glutamic acid); (β-alanine)-(glycine)3-(serine)-(glycine)-(glutamic acid); or (β-alanine)-(glycine)4-(serine)-(glutamic acid).

In some embodiments, M^{A} comprises (glycine)4-(serine). In some embodiments, the peptide moiety comprises (serine)-(glycine)4.

In some embodiments, M^{A} comprises (β-alanine)-(glycine)4-(serine) wherein the serine is at any position along the peptide chain.

In some embodiments, M^{A} comprises (glycine)4-(serine)-(glutamic acid) wherein the serine is at any position along the peptide chain.

In some embodiments, M^{A} comprises (β-alanine)-(glycine)4-(serine)-(glutamic acid) wherein the serine is at any position along the peptide chain.

In some embodiments, M^{A} comprises (glutamic acid)-(glycine)1-4, wherein: the M^{A} is attached to H^{1B} via one of the glutamic acid; M^{A} is attached to T^{A} via the glycine; and M^{A} is attached to H^{1A} via the glutamic acid.

In some embodiments, M^{A} comprises

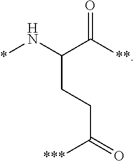

In some embodiments, M^{A} comprises

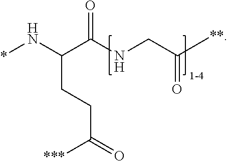

In some embodiments, M^{A} comprises (glutamic acid)-(glycine)₄, wherein: the M^{A} is attached to H^{1B} via one of the glutamic acid; M^{A} is attached to T^{A} via one of the glycine; and M^{A} is attached to H^{1A} via the glutamic acid.

In some embodiments, M^{A} comprises

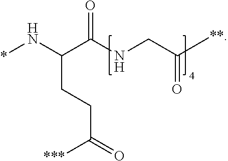

In some embodiments, M^{A} comprises

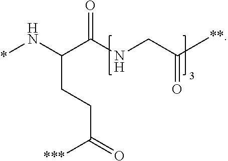

In some embodiments, M^{A} comprises (glutamic acid)-(glycine), wherein: the M^{A} is attached to H^{1B} via the glutamic acid; M^{A} is attached to T^{A} via the glycine; and M^{A} is attached to H^{1A} via the glutamic acid.

In some embodiments, the peptide moiety comprises

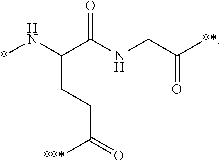

In some embodiments, M^{A} comprises

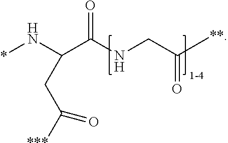

In some embodiments, M^{A} comprises (glycine)₁₋₄-(glutamic acid), wherein M^{A} is attached to H^{1B} via one of the glycine; M^{A} is attached to T^{A} via the glutamic acid; and M^{A} is attached to H^{1A} via the glutamic acid.

In some embodiments, M^{A} comprises

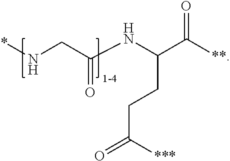

In some embodiments, M^{A} comprises (glycine)₄-(glutamic acid), wherein M^{A} is attached to H^{1B} via the the glutamic acid; M^{A} is attached to T^{A} via glycine; and M^{A} is attached to H^{1A} via the glutamic acid.

In some embodiments, M^{A} comprises

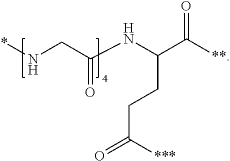

In some embodiments, M^{A} comprises more preferably (glycine)-(glutamic acid), wherein M^{A} is attached to H^{1B} via the glycine; M^{A} is attached to M^{A} via the glutamic acid; and M^{A} is attached to H^{1A} via the glutamic acid.

In some embodiments, M^{A} comprises

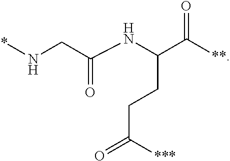

In some embodiments, M^{A} comprises (glycine)₁₋₄-(serine), wherein: M^{A} is attached to H^{1B} via one of the glycine; M^{A} is attached to M^{A} via the serine; and M^{A} is attached to H^{1B} via the serine.

In some embodiments, M^{A} comprises

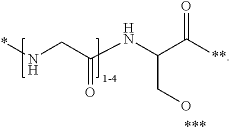

In some embodiments, M^{A} comprises (glycine)-(serine), wherein: M^{A} is attached to H^{1B} via the glycine; M^{A} is attached to T^{A} via the serine; and M^{A} is attached to H^{1A} via the serine.

In some embodiments, M^{A} comprises

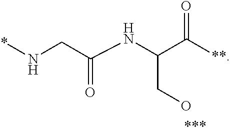

In some embodiments, M^{A} comprises (glycine)4-(serine) wherein: M^{A} is attached to H^{1B} via one of the glycine; M^{A} is attached to T^{A} via the serine; and M^{A} is attached to H^{1A} via the serine.

In some embodiments, M^{A} comprises

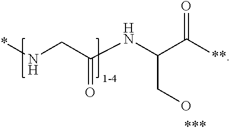

In some embodiments, M^{A} comprises (serine)-(glycine)₁₋₄, wherein: M^{A} is attached to H^{1B} via the serine; M^{A} is attached to T^{A} via one of the glycine; and M^{A} is attached to H^{1A} via the serine.

In some embodiments, M^{A} comprises

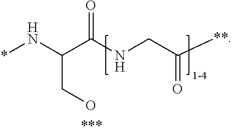

In some embodiments, M^{A} comprises (serine)-(glycine)₄, wherein: M^{A} is attached to H^{1B} via the serine; M^{A} is attached to T^{A} via one of the glycine; and M^{A} is attached to H^{1A} via the serine.

In some embodiments, M^{A} comprises

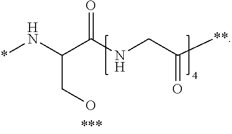

In some embodiments, M^{A} comprises (serine)-(glycine), wherein: M^{A} is attached to H^{1B} via the serine; M^{A} is attached to T^{A} via the glycine; and M^{A} is attached to H^{1A} via the serine.

In some embodiments, M^{A} comprises

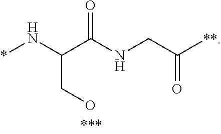

In some embodiments, M^{A} comprises (β-alanine)-(glycine)₁₋₄-(serine), wherein: M^{A} is attached to H^{1B} via the β-alanine; M^{A} is attached to T^{A} via the serine; and M^{A} is attached to H^{1A} via the serine.

In some embodiments, M^{A} comprises

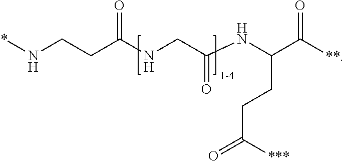

In some embodiments, M^{A} comprises (β-alanine)-(glycine)₄-(serine), wherein: M^{A} is attached to H^{1B} via the β-alanine; M^{A} is attached to T^{A} via the serine; and M^{A} is attached to H^{1A} via the serine.

In some embodiments, M^{A} comprises

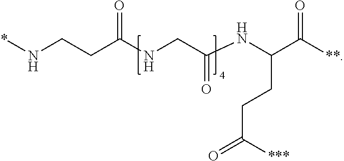

In some embodiments, the peptide moiety comprises (β-alanine)-(glycine)-(glutamic acid), wherein: the peptide moiety is attached to H^{1B} via the β-alanine; the peptide moiety is attached to T^{A} when present, via the glutamic acid; and the peptide moiety is attached to H^{1A} when present, via the glutamic acid.

In some embodiments, the peptide moiety comprises

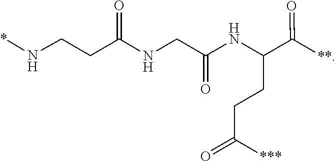

In some embodiments, M^{A} comprises

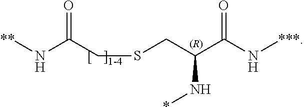

In some embodiments, M^{A} comprises

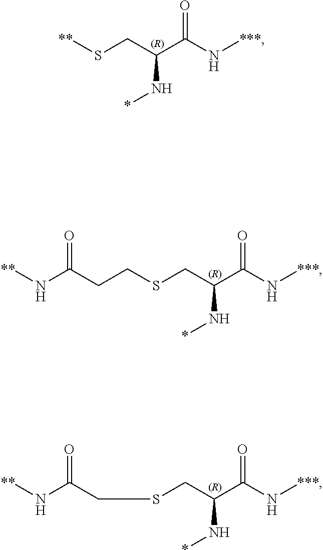

It is understood that for embodiments of M^{A}, * indicates attachment to H^{1B}; ** indicates attachment to T^{A}, and *** indicates attachment to H^{1A}

In some embodiments, the hydrophilic group T^{A} included in the conjugates or the compound-linker constructs of the disclosure is a water-soluble and substantially non-antigenic polymer. Examples of the hydrophilic group, include, but are not limited to, polyalcohols, polyethers, polyanions, polycations, polyphosphoric acids, polyamines, polysaccharides, polyhydroxy compounds, polylysines, and derivatives thereof. In some embodiments, one end of the hydrophilic group can be functionalized so that it can be covalently attached to the M^{A} linker (e.g., to an amino acid in the M^{A} linker) by means of a non-cleavable linkage or via a cleavable linkage. In some embodiments, functionalization can be, for example, via an amine, thiol, NHS ester, maleimide, alkyne, azide, carbonyl, or other functional group. In some embodiments, the other terminus (or termini) of the hydrophilic group will be free and untethered. In some embodiments, by “untethered”, it is meant that the hydrophilic group will not be attached to another moiety, such as D or a Drug Unit, or other components of the conjugates or compound-linker constructs of the disclosure. In some embodiments, the free and untethered end of the hydrophilic group may include a methoxy, carboxylic acid, alcohol or other suitable functional group. In some embodiments, the methoxy, carboxylic acid, alcohol, or other suitable functional group acts as a cap for the terminus or termini of the hydrophilic group.

In some embodiments, a cleavable linkage refers to a linkage that is not substantially sensitive to cleavage while circulating in the plasma but is sensitive to cleavage in an intracellular or intratumoral environment. In some embodiments, a non-cleavable linkage is one that is not substantially sensitive to cleavage in any biological environment. In some embodiments, chemical hydrolysis of a hydrazone, reduction of a disulfide, and enzymatic cleavage of a peptide bond or glycosidic linkage are examples of cleavable linkages. In some embodiments, exemplary attachments of the hydrophilic group are via amide linkages, ether linkages, ester linkages, hydrazone linkages, oxime linkages, disulfide linkages, peptide linkages, or triazole linkages. In some embodiments, the attachment of the hydrophilic group to the M^{A} linker (e.g., to an amino acid in the M^{A} linker) is via an amide linkage.

In some embodiments wherein the conjugate or the compound-linker construct of the disclosure comprises more than one hydrophilic groups, the multiple hydrophilic groups may be the same or different chemical moieties (e.g., hydrophilic groups of different molecular weight, number of subunits, or chemical structure). In some embodiments, the multiple hydrophilic groups can be attached to the M^{A} linker at a single attachment site or different sites.

In some embodiments, the addition of the hydrophilic group may have two potential impacts upon the pharmacokinetics of the resulting conjugate. In some embodiments, the desired impact is the decrease in clearance (and consequent in increase in exposure) that arises from the reduction in non-specific interactions induced by the exposed hydrophobic elements of the drug or drug-linker. In some embodiments, the undesired impact is the decrease in volume and rate of distribution that may arise from the increase in the molecular weight of the conjugate. In some embodiments, increasing the molecular weight of the hydrophilic group increases the hydrodynamic radius of a conjugate, resulting in decreased diffusivity that may diminish the ability of the conjugate to penetrate into a tumor. Because of these two competing pharmacokinetic effects, it may be desirable to use a hydrophilic group that is sufficiently large to decrease the conjugate clearance thus increasing plasma exposure, but not so large as to greatly diminish its diffusivity, which may reduce the ability of the conjugate to reach the intended target cell population.

In some embodiments, the hydrophilic group, includes, but is not limited to, a sugar alcohol (also known as polyalcohol, polyhydric alcohol, alditol or glycitol, such as inositol, glycerol, erythritol, threitol, arabitol, xylitol, ribitol, galactitol, mannitol, sorbitol, and the like) or a derivative thereof (e.g., amino polyalcohol), carbohydrate(e.g., a saccharide), a polyvinyl alcohol, a carbohydrate-based polymer (e.g., dextrans), a hydroxypropylmethacrylamide (HPMA), a polyalkylene oxide, and/or a copolymer thereof.

In some embodiments, T^{A} comprises a plurality of hydroxyl (“—OH”) groups, such as moieties that incorporate monosaccharides, oligosaccharides, polysaccharides, and the like.

In some embodiments, T^{A} comprises a plurality of —(CR₅₈OH)—groups, wherein R₅₈ is —H or C₁₋₈ alkyl.

In some embodiments, T^{A} is —OH.

In some embodiments, T₁is

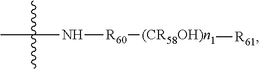

n₁ is an integer from 0 to about 6;
each R₅₈ is independently —H or C₁₋₈ alkly;
R₆₀ is a bond, a C₁₋₆ alkyl linker, or —CHR₅₉—; wherein R₅9 is —H, C₁₋₈ alkyl, cycloalkyl, or arylalkyl;
R⁶¹ is CH₂OR₆₂, COOR₆₂, —(CH₂)n₂COOR₆₂, or a heterocycloalkyl substituted with one or more hydroxyl;
R₆₂ is —H or C₁₋₈ alkyl; and
n₂ is an integer from 1 to about 5.

In some embodiments, T^{A} comprises a glucosyl-amine, a di- amine, or a tri- amine.

In some embodiments, T^{A} comprises one or more of the following fragments or a stereoisomer thereof:

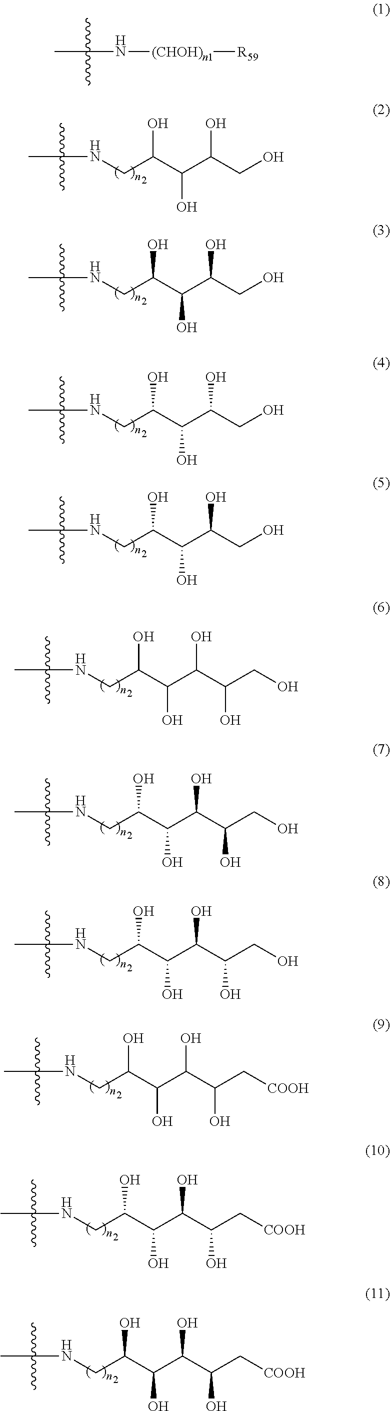

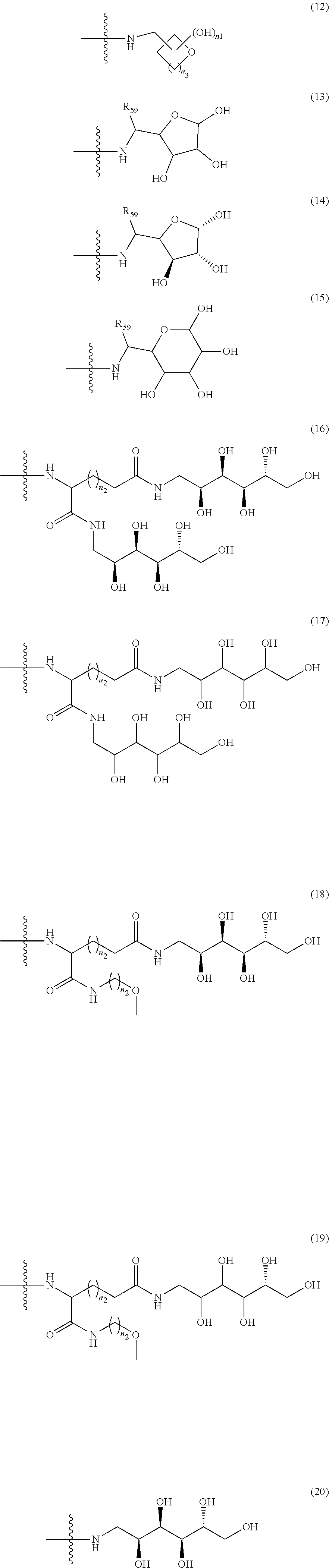

wherein: R₅₉ is —H, C_{1 8} alkyl, cycloalkyl, or arylalkyl; n₁ is an integer from 1 to about 6; n2 is an integer from 1 to about 5; and n3 is an integer from about 1 to about 3.

In some embodiments, n3 is 2 or 3; n₁ is 1, 2, or 3; n₂ is 1; or R₅₉ is hydrogen.

In some embodiments, T^{A} is

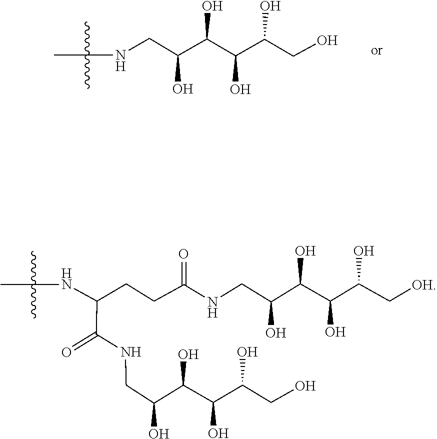 [SMILES: *NC[C@H](O)[C@@H](O)[C@H](O)[C@H](O)CO ; *NC(CCC(=O)NC[C@H](O)[C@@H](O)[C@H](O)[C@H](O)CO)C(=O)NC[C@H](O)[C@@H](O)[C@H](O)[C@H](O)CO]

In some embodiments, T^{A} is

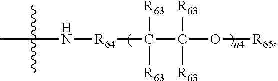

wherein
n4 is an integer from 1 to about 25;
each R₆₃ is independently hydrogen or C₁ s alkyl;
R₆₄ is a bond or a C_{1 8} alkyl linker;
R₆₅ is H, C₁₋₈ alkyl, —(CH2)n2COOR₆₂ or —(CH₂)n2COR₆₆;
R₆₂ is H

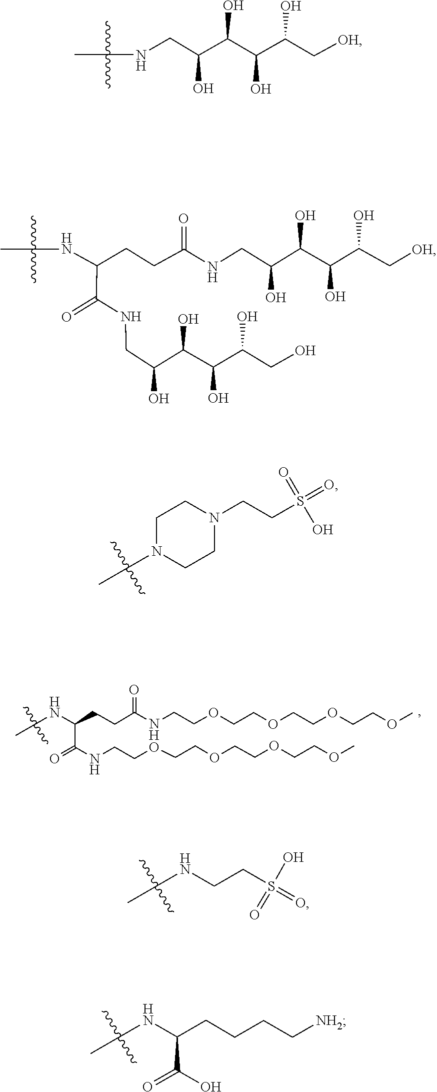

and
n2 is an integer from 1 to about 5.

In some embodiments, T^{A}

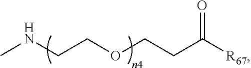

wherein R₆₇ is (1)—OH,

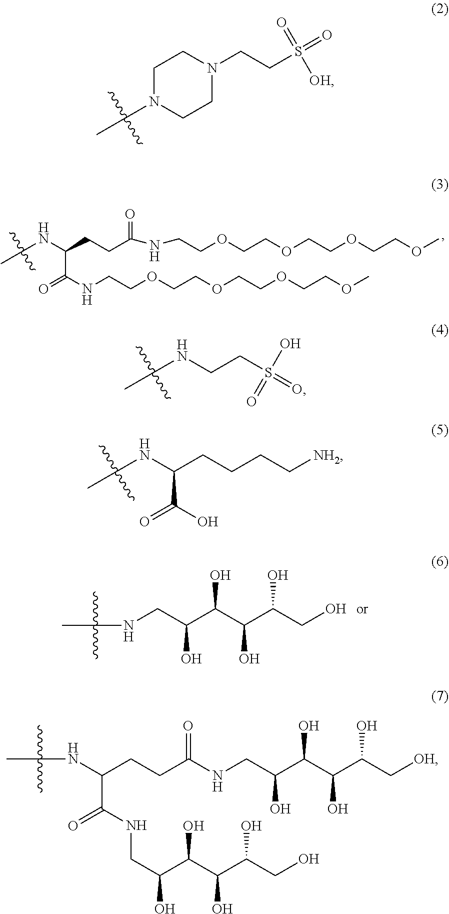

wherein n4 is an integer from about 2 to about 20, from about 4 to about 16, from about 6 to about 12, from about 8 to about 12.

In some embodiments, T^{A} is

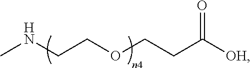

wherein n₄ is an integer from about 2 to about 24, from about 4 to about 16, from about 6 to about 12, from about 8 to about 12.

In some embodiments, n4 is 6, 7, 8, 9, 10, 11, or 12; preferably 8 or 12; more preferably 8.

In some embodiments, T^{A} is

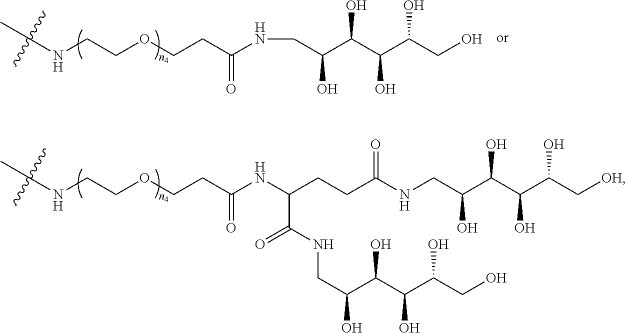

wherein n₄ is an integer from about 2 to about 24, from about 4 to about 16, from about 6 to about 12, from about 8 to about 12.

In some embodiments, n4 is 6, 7, 8, 9, 10, 11, or 12; preferably 8 or 12; more preferably 8.

In some embodiments, examples of hydrophilic groups that are suitable for the conjugates, scaffolds, and methods disclosed herein can be found in e.g., U.S. Pat. No. 8,367,065 column 13; U.S. Pat. No. 8,524,696 column 6; WO2015/057699 and WO 2014/062697, the contents of each of which are hereby incorporated by reference in their entireties.

In some embodiments, the linker L¹ has the structure:

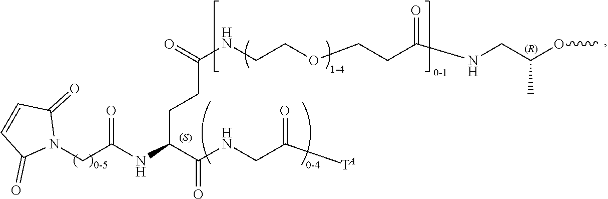

wherein T^{A} is the same as defined herein, and [symbol] denotes attachment to a payload, for example the STING agonist as defined herein.

In some embodiments, the linker L¹ has the structure:

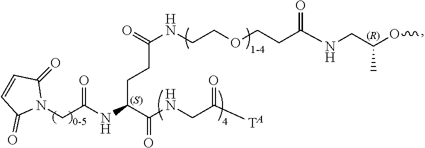

wherein T^{A} is the same as defined herein, and [symbol] denotes attachment to a payload, for example the STING agonist as defined herein.

In some embodiments, the linker L¹ has the structure:

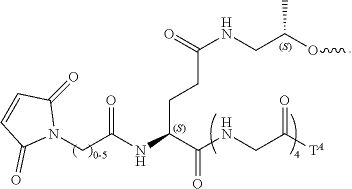

wherein T^{A} is the same as defined herein, and [symbol] denotes attachment to a payload, for example the STING agonist as defined herein.

In some embodiments, the linker L¹ has the structure:

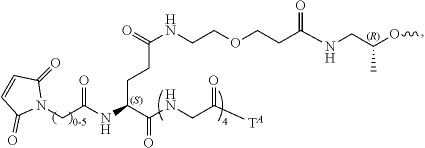

wherein T^{A} is the same as defined herein, and [symbol] denotes attachment to a payload, for example the STING agonist as defined herein.

In some embodiments, the linker L¹ has the structure selected from the group consisting of:

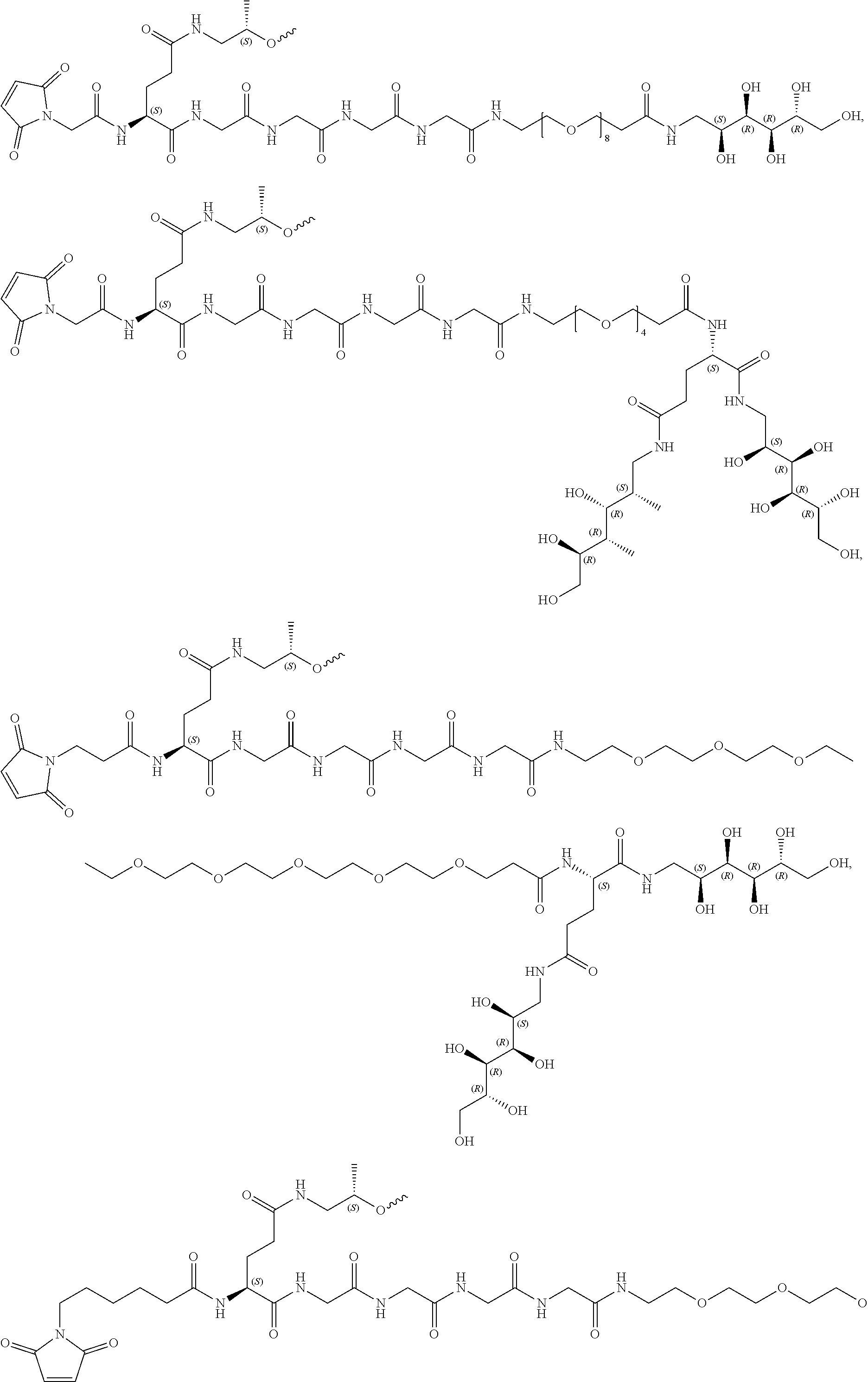

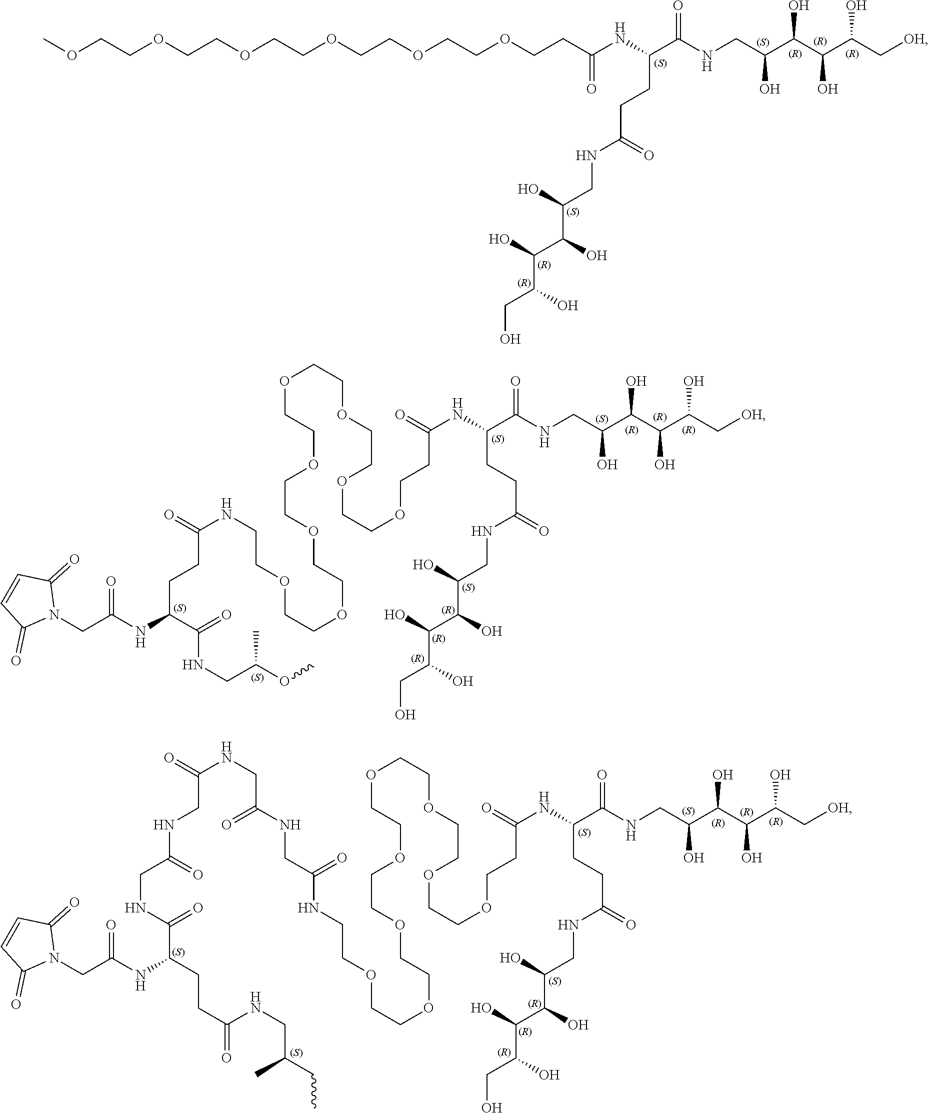

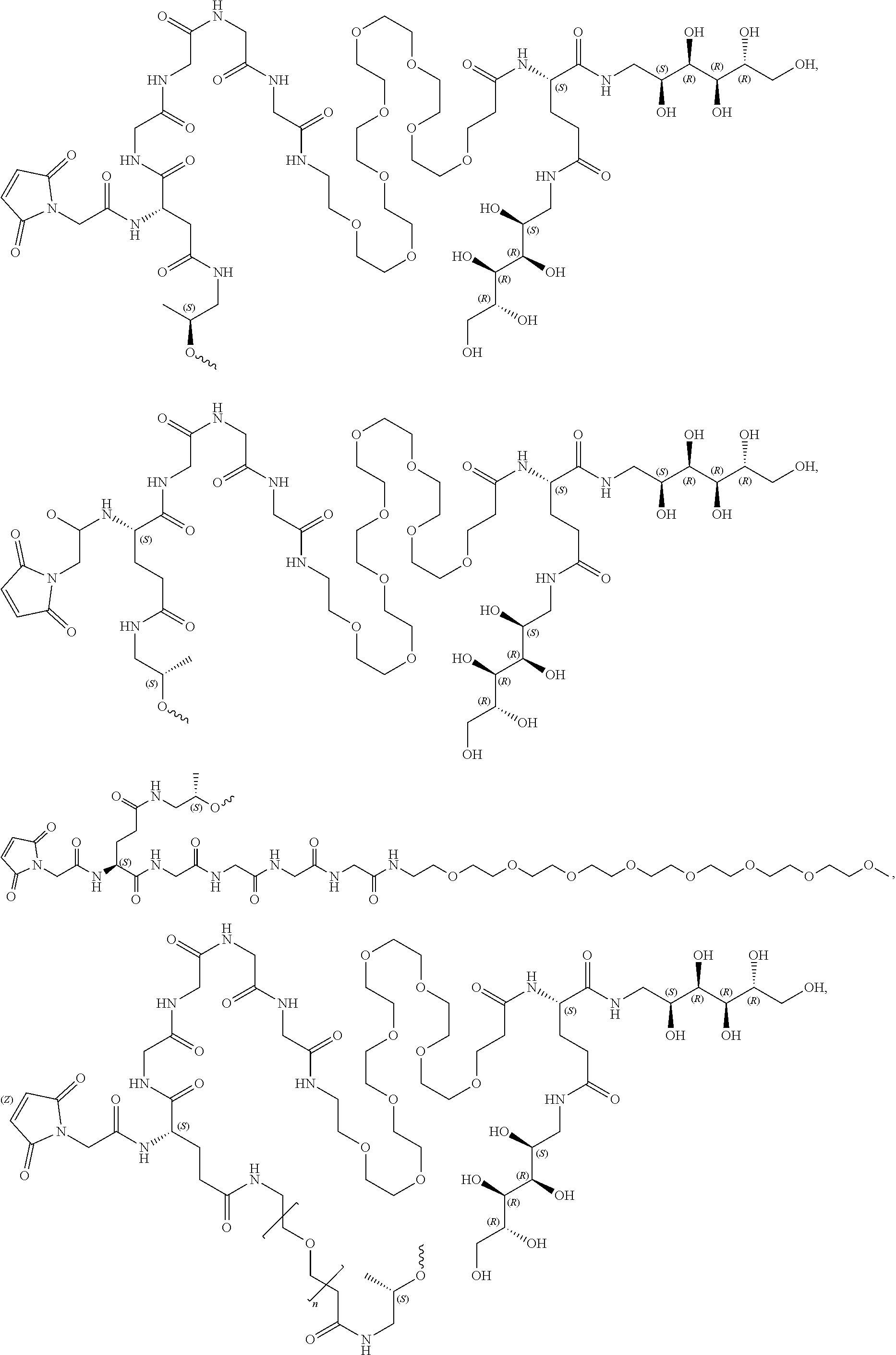

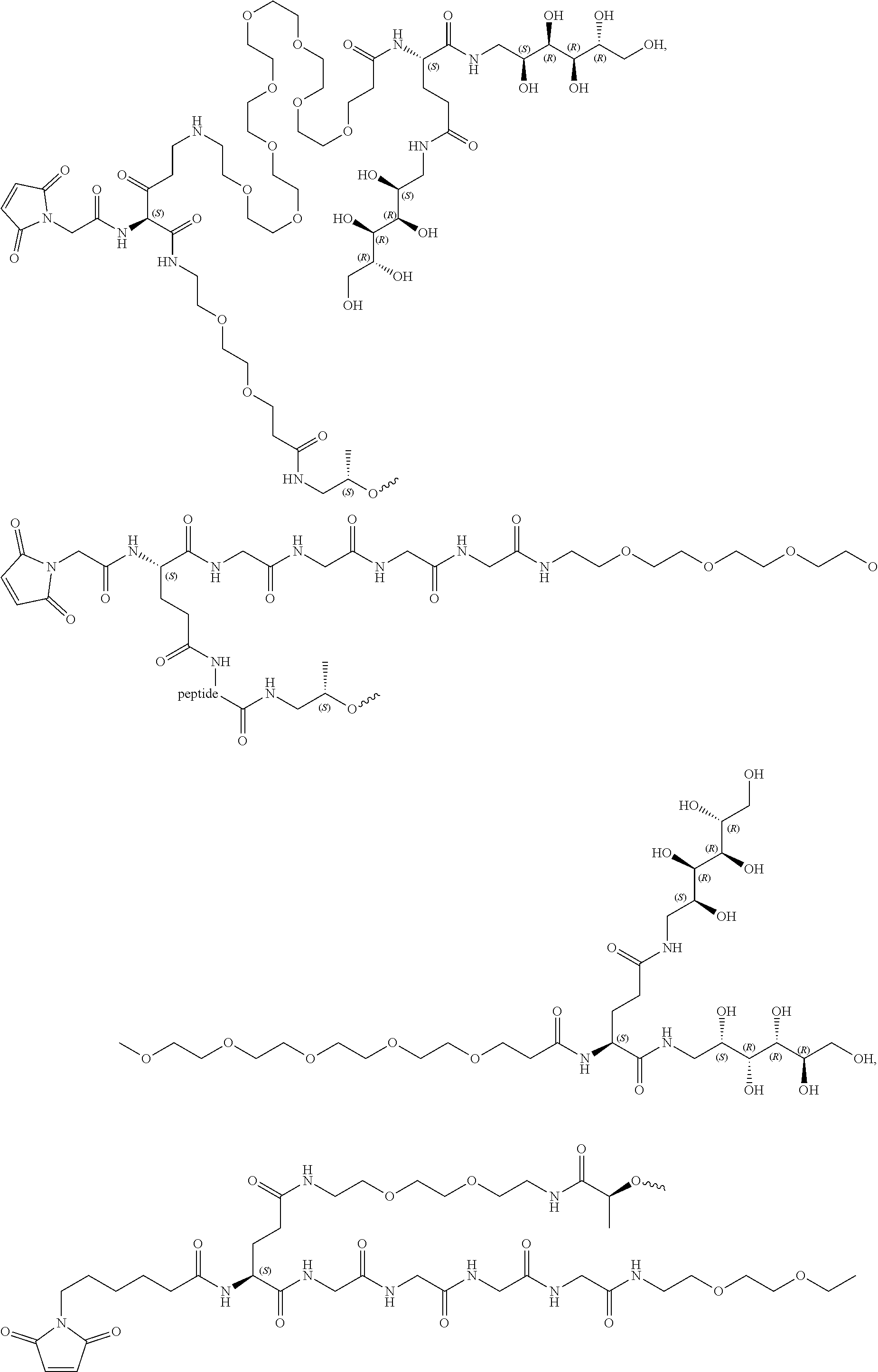

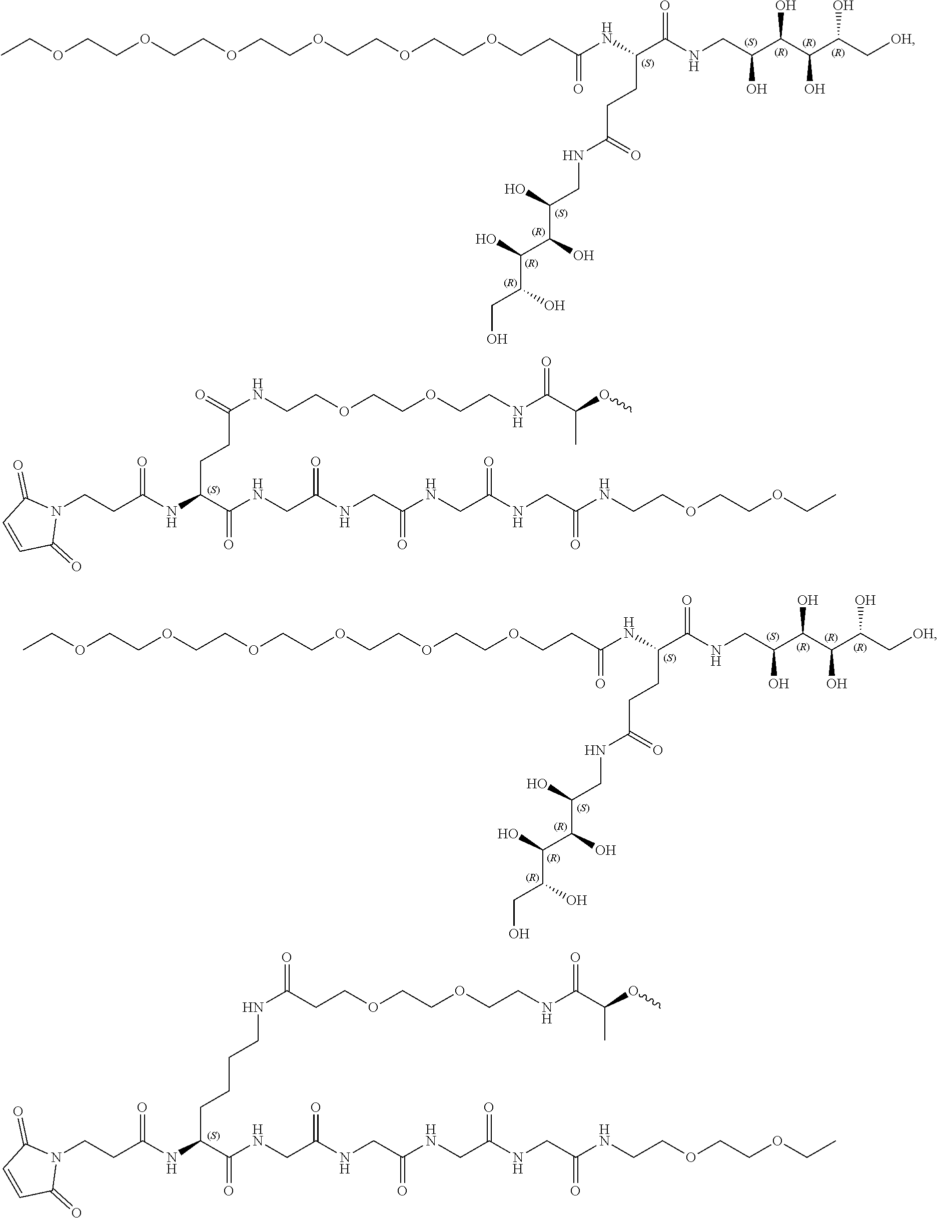

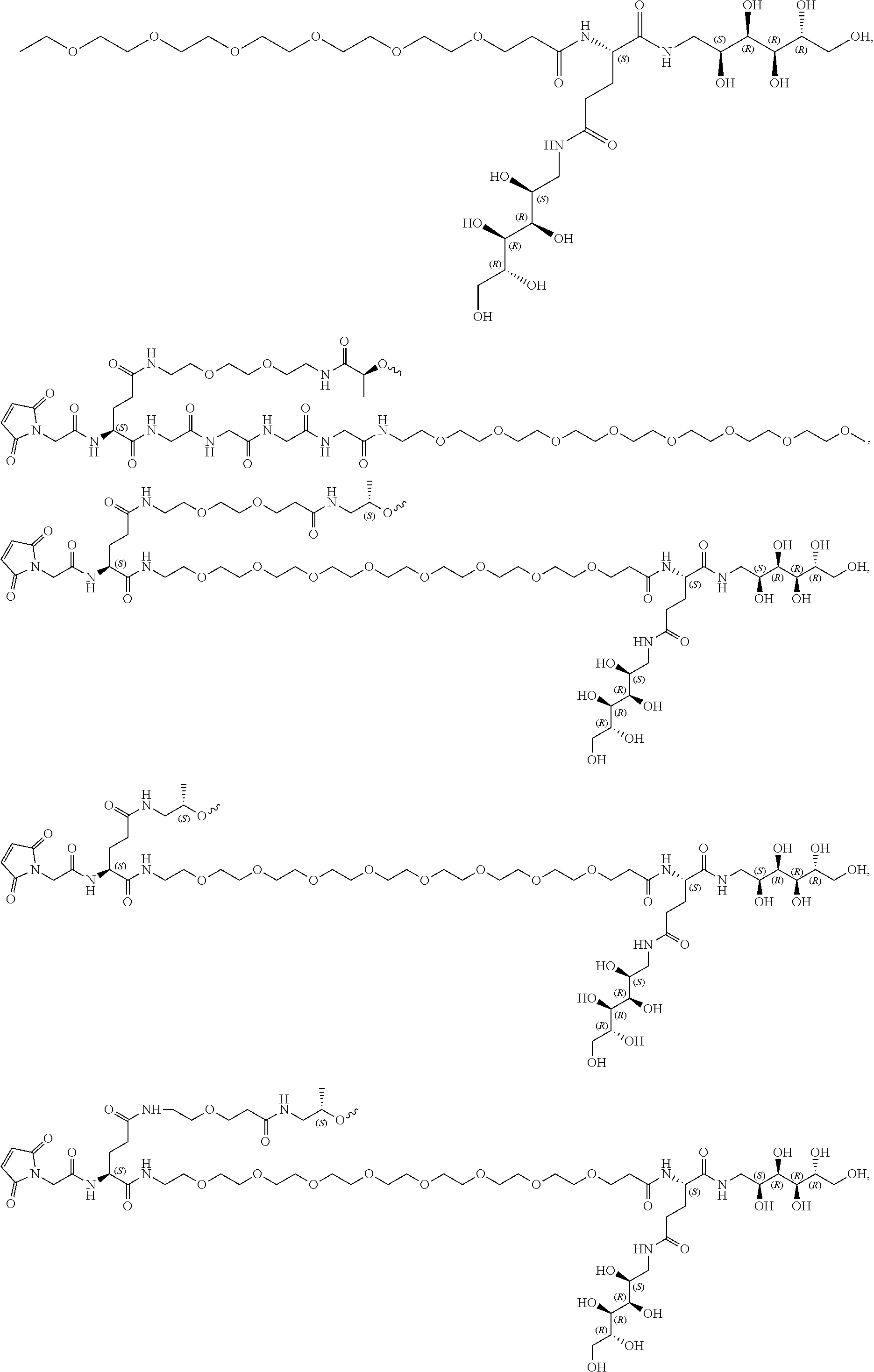

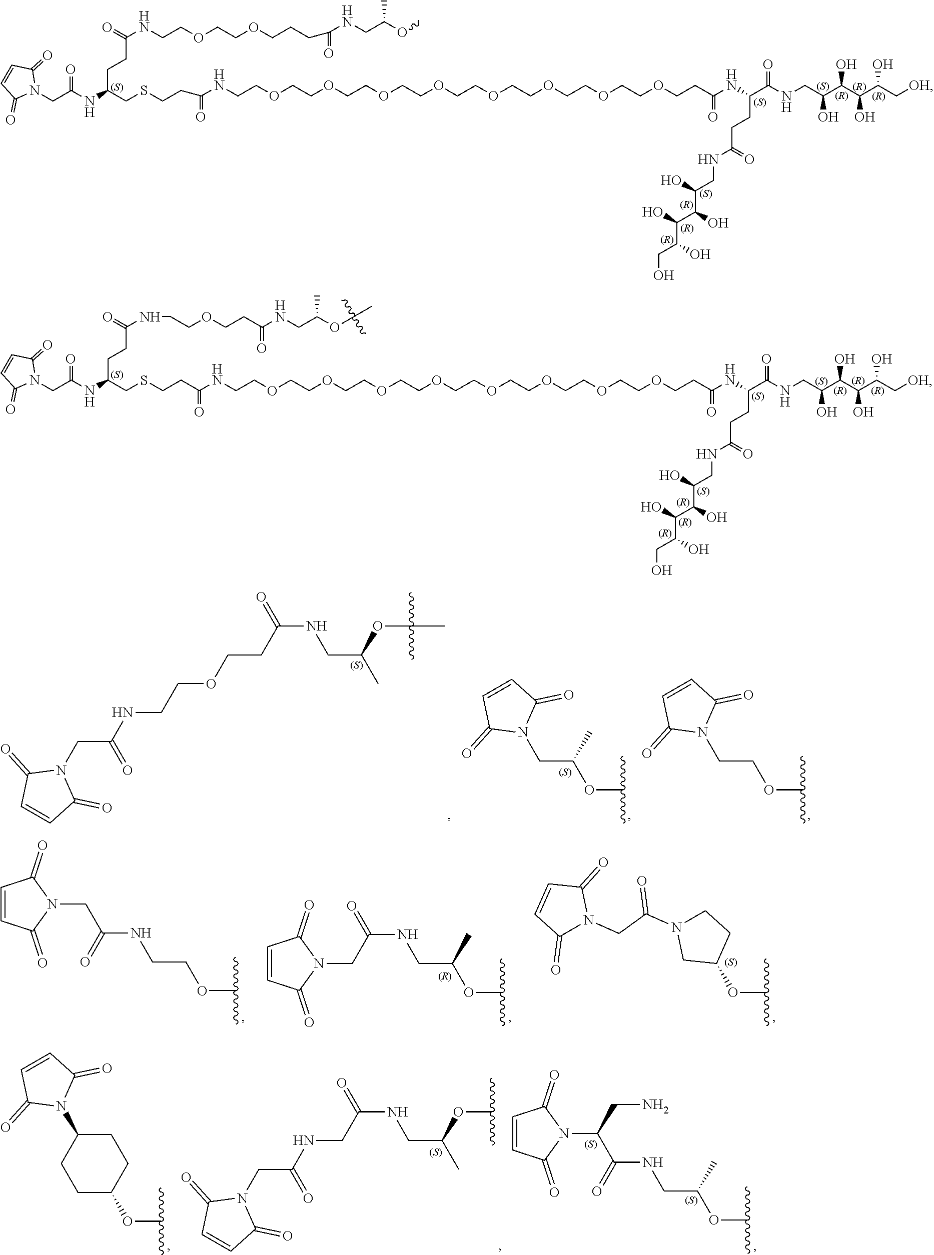

wherein [symbol] denotes attachment to a payload, for example the STING agonist as defined herein.

In some embodiments, when L^{c} is absent, the linker L¹ is selected from the group consisting of:

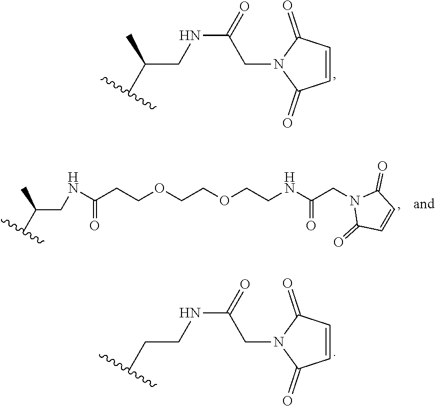 [SMILES: *[C@@H](C)CNC(=O)CN1C(=O)C=CC1=O ; *[C@@H](C)CNC(=O)CCOCCOCCNC(=O)CN1C(=O)C=CC1=O ; *CCNC(=O)CN1C(=O)C=CC1=O]

In some embodiments, the linker L¹ has the structure H^{1A}-L^{C}-H^{1B} and is selected from the group consisting of:

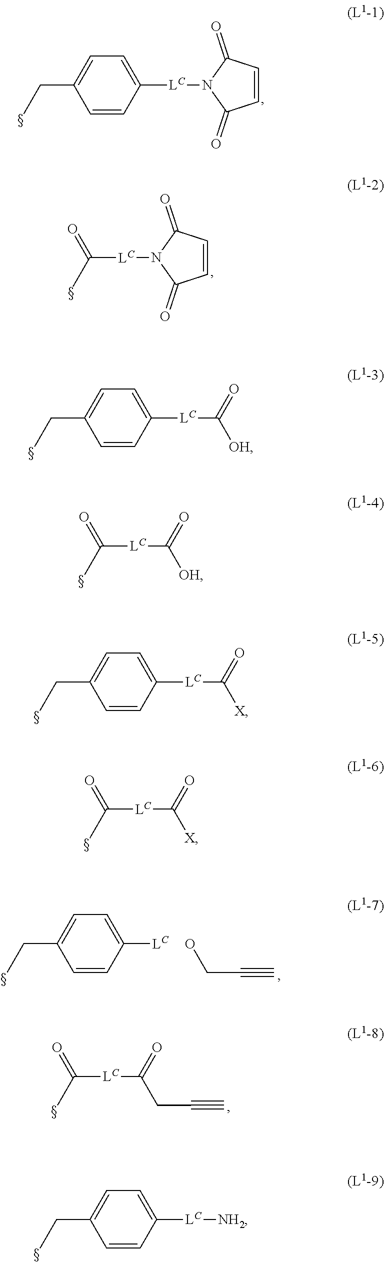

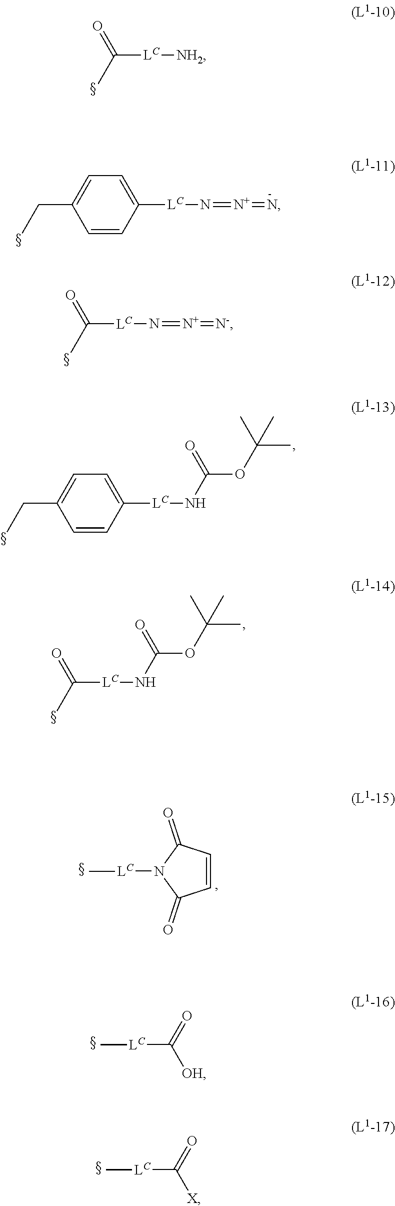

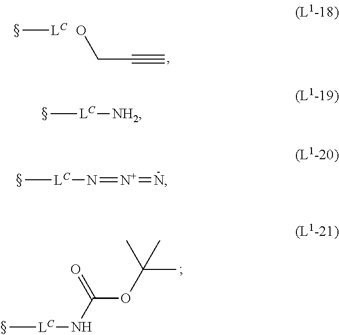

wherein
L^{c} is a chain L^{c} of units selected from a linear or branched polyethylene glycol chain, a sequence of amino acids, and a linear or branched C₁-C₁₀-alkyl chain, wherein each substitutable carbon or heteroatom of the aforementioned units may be unsubstituted or substituted with one or more, same or different substituents selected from deuterium halogen, OH, ═O, C₁-C₆-alkyl, C₁-C₆-haloalkyl, C₃-C₆-cycloalkyl, C₃-C₆-halocycloalkyl, C₁-C₆-alkoxy, and C₁-C₆-haloalkoxy; and
§ marks the connection to the STING agonist moiety; and
X represents a leaving group selected from

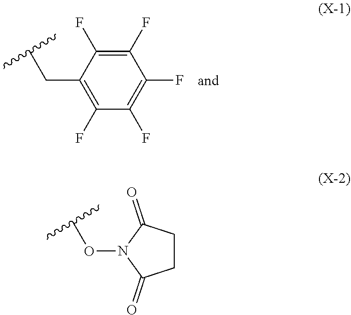 [SMILES: *Cc1c(F)c(F)c(F)c(F)c1F ; "and" ; *ON1C(=O)CCC1=O]

and wherein preferably L^{c} is selected from the group consisting of

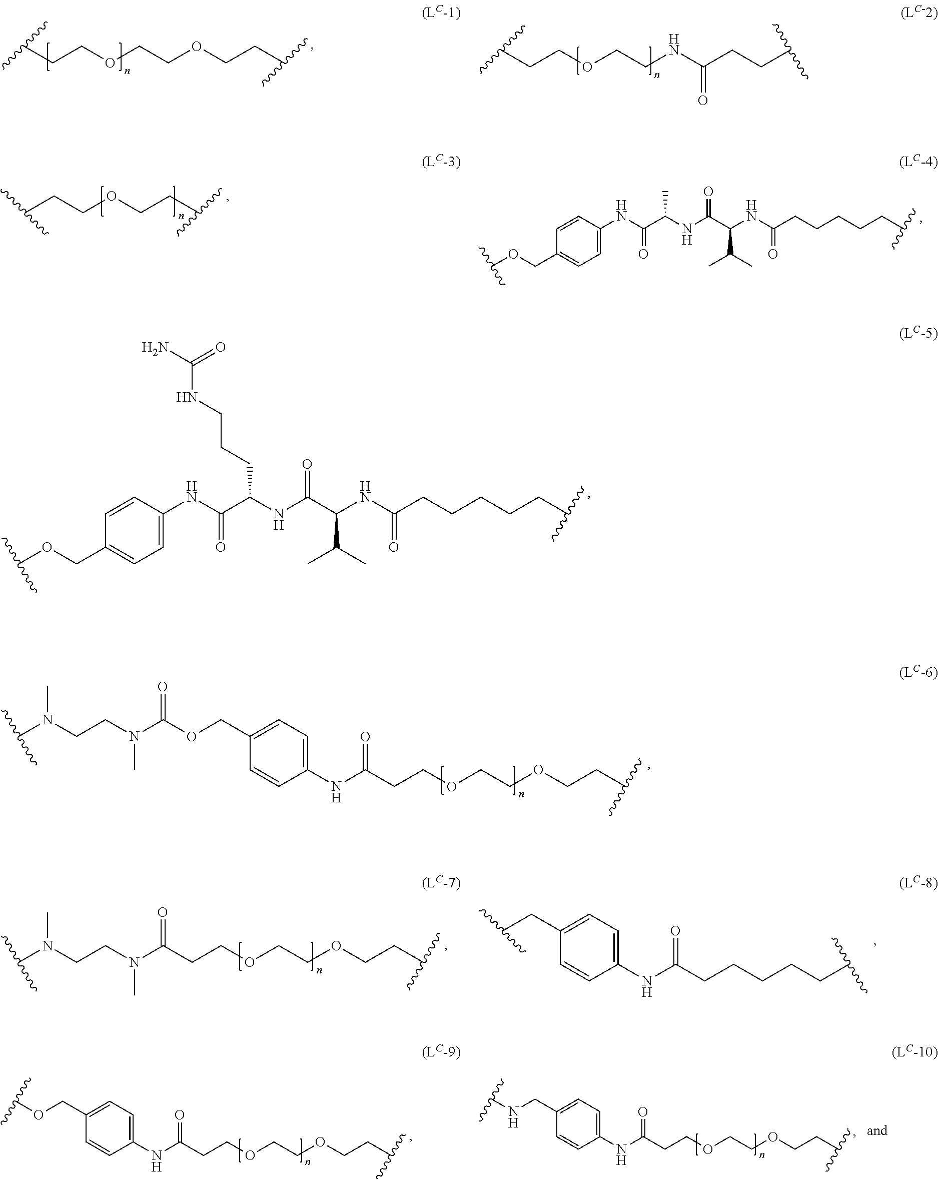

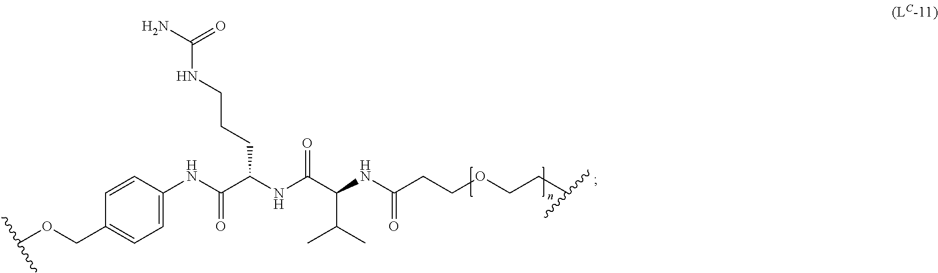

wherein n is 0, 1, 2, 3, 4, 5, 6, 7, or 8.

In some embodiments, L^{c} is selected from:
—C(═O)(CH₂)ₘ—**; —C(═O)(CH₂)ₘ (CH₂)ₘ—**; —C(═O)XₐX_{b}C(═O)(CH₂)ₘ—**; —C(═O)XₐX_{b} C(═O)(CH₂)ₘO(CH₂)ₘ—**; —C(═O)XₐX_{b}C(═O)(CH₂)ₘO(CH₂)ₘC(═O)—**; C(═O)X₄C(═O)NH(CH₂)ₘNHC(═O)(CH₂)ₘO(CH₂)ₘ—**; —C(═O)X₅C(═O)(CH₂)ₘNHC(═O)(CH₂)ₘ—**; —C(═O)(CH₂)ₘNHC(═O)XₐX_{b}C(═O)(CH₂)ₘ—**; —C(═O)O(CH₂)ₘNHC(═O)XₐX_{b}C(═O)(CH₂)ₘ—**; —C(═O)(CH₂)ₘNHC(═O)(CH₂)ₘO)ₙ (CH₂)ₘ—**; —C(═O)O(CH₂)ₘNHC(═O)(CH₂)ₘ—**; —C(═O)O(CH₂)ₘNHC(═O)(CH₂)ₘO(CH₂)ₘ—**; —C(═O)O(CH₂)ₘNHC(═O)XₐX_{c}(═O)(CH₂)ₘO(CH₂)ₘ—**; —X₂C(═O)(CH₂)ₘ—**-; —C(═O)X₅C(═O)(CH₂)ₘNHC(═O)(CH₂)ₘ—**; —C(═O)O(CH₂)ₘNHC(═O)XₐX_{b} C(═O)(CH₂)ₘO(CH₂)ₘC(═O)—**; —C(═O)O(CH₂)ₘNHC(═O)X₄C(═O)NH(CH₂)ₘNHC(═O)(CH₂)ₘO(CH₂)ₙ—**; —C(═O)O((CH₂)ₘO)ₙ(CH₂)ₘNHC(═O)X₅C(═O)(CH₂)ₘ—**; —C(═O)O((CH₂)ₘO)ₙ(CH₂)ₘNHC(═O)X₅C(═O)(CH₂)ₘNHC(═O)(CH₂)ₘ—**; —C(═O)O((CH₂)ₘO)ₙ(CH₂)ₘNHC(═O)X₅C(═O)(CH₂)ₘX_{c}(CH₂)ₘ—**; C(═O)O((CH₂)ₘO)ₙ(CH₂)ₘNHC(═O)X₅C(═O)(CH₂)ₘO)ₙ (CH₂)ₘ—**; —C(═O)O((CH₂)ₘO)ₙ(CH₂)ₘNHC(═O)X₅ C(═O)((CH₂)ₘO)ₙ(CH₂)ₘNHC(═O)(CH₂))ₘ—**; —C(═O)O(CH₂)ₘO)ₙ (CH₂)ₘNHC(═O)X₅ C(═O)(CH₂)ₘO)ₙ (CH₂)ₘNHC(═O)(CH₂)ₘX_{c}(CH₂)ₘ—**; —C(═O)O((CH₂)ₘO)ₙ(CH₂)ₘNHC(═O))X₅C(═O)(CH₂)ₘO)ₙ (CH₂)ₘX_{c}(CH₂)ₘ—**; —C(═O)O((CH₂)ₘO)ₙ(CH₂)ₘNHC(═O)X₅C(═O)(CH₂)ₘNHC(═O)((CH₂)ₘO)ₙ(CH₂)ₘ—**; —C(═O)O((CH₂)ₘO)ₙ(CH₂)ₘNHC(═O)X₅C(═O)(CH₂)ₘNHC(═O)(CH₂)ₘO)ₙ (CH₂)ₘX_{c}(CH₂)ₘ—**; —C(═O)O((CH₂)ₘO)ₙ(CH₂)ₘNHC(═O)X(CH₂)ₘX_{c}(CH₂)ₘ—**; —C(═O)O((CH₂)ₘO)ₙ(CH₂)ₘNHC(═O)X₅((CH₂)ₘO)ₙ(CH₂)ₘ—**; —C(═O)O((CH₂)ₘO)ₙ(CH₂)ₘNHC(═O)X₅(CH₂)ₘO)ₙ (CH₂)ₘNHC(═O)(CH₂)ₘ—**; —C(═O)O((CH₂)ₘO)ₙ(CH₂)ₘNHC(═O)X₅(CH₂)ₘO)ₙ (CH₂)ₘNHC(═O)(CH₂)ₘX_{c}(CH₂)ₘ—**; —C(═O)O((CH₂)ₘO)ₙ(CH₂)ₘNHC(═O)X₅(CH₂)ₘO)ₙ (CH₂)ₘX_{c}(CH₂)ₘ—**; —C(═O)O((CH₂)ₘO)ₙ(CH₂)ₘNHC(═O)X₅(CH₂)ₘNH(CH₂)ₘO)ₙ (CH₂)ₘ—**; —C(═O)O((CH₂)ₘO)ₙ(CH₂)ₘNHC(═O)X₅C(═O)(CH₂)ₘNH(CH₂)ₘO)ₙ (CH₂)ₘX_{c}(CH₂)ₘ—**; —C(═O)O(CH₂)ₘO)ₙ (CH₂)ₘNHC(═O)X₅(CH₂)ₘ—**; —C(═O)O(CH₂)ₘ—**; —C(═O)O((CH₂)ₘO)ₙ(CH₂)ₘ—**; —C(═O)O(CH₂)ₘNH(CH₂)ₘ—**; —C(═O)O(CH₂)ₘNH(CH₂)ₘC(═O)XₐXC(═O)—**; —C(═O)O(CH₂)ₘX_{c}(CH₂)ₘ—**; —C(═O)O(CH₂)ₘNHC(═O)XₐX_{b} C(═O)CH₂)ₘO)ₙ (CH₂)ₘC(═O)—**; —C(=)O((CH₂)ₘO)ₙ(CH₂)ₘX_{c}(CH₂)ₘ—**; —C(═O)O((CH₂)ₘO)ₙ(CH₂)ₘNHC(═O)(CH₂)ₘ—**; —C(═O)O(CH₂)ₘNHC(═O(CH₂)ₘX_{c}(CH₂)ₘ—**; —C(═O)O((CH₂)ₘO)ₙ(CH₂)ₘNHC(═O)(CH₂)ₘX_{c}(CH₂)ₘ—**; —C(═O)O((CH₂)ₘO)ₙX₃(CH₂)ₘ—**; —C(═O)O((CH₂)ₘO)ₙ(CH₂)ₘX₃(CH₂)ₘ—**; —C(═O)O((CH₂)ₘO)ₙ(CH₂)ₘC(═O)NH(CH₂)ₘ—**; —C(═O)O(CH₂)ₘC(R¹²)₂—**; —C(═O)O(CH₂)ₘSSC(R¹²)₂(CH₂)ₘC(═O)NR¹²(CH₂)ₘNR¹²C(═O)(CH₂)ₘO(CH₂)ₘ—**; —C(═O)O(CH₂)ₘSSC(R¹²)₂(CH₂)ₘC(═O)NR¹²(CH₂)ₘNR¹²C(═O)(CH₂)ₘ—**; —C(═O)O(CH₂)ₘC(═O)NH(CH₂)ₘ—**; —C(═O)((CH₂)ₘO)ₙ(CH₂)ₘ—**; —C(═O)(CH₂)ₘNH(CH₂)ₘ—**; —C(═O)(CH₂)ₘNH(CH₂)ₘC(═O)XₐX_{b} C(═O)—**; —C(═O)(CH₂)ₘX_{c}(CH₂)ₘ—**; —C(═O)((CH₂)ₘO)ₙ(CH₂)ₘX_{c}(CH₂)ₘ—**; —C(═O)(CH₂)ₘNHC(═O)(CH₂)ₘ—**; —C(═O)(CH₂)ₘO)ₙ (CH₂)ₘNHC(═O)(CH₂)ₘ—**; —C(═O)(CH₂)ₘNHC(═O(CH₂)ₘX_{c}(CH₂)ₘ—**; —(CH₂)ₘNHC(═O)XₐX_{b} C(═O)(CH₂)ₘ—**; —(CH₂)ₘ(CHOH)(CH₂)ₘNHC(═O)XₐX_{b} C(═O)(CH₂)ₘ**; —C(═O)((CH₂)ₘO)ₙ(CH₂)ₘNHC(═O)(CH₂)ₘX_{c}(CH₂)ₘ—**; —C(═O)((CH₂)ₘO)nXc(CH₂)ₘ—**; —C(═O)((CH₂)ₘO)ₙ(CH₂)ₘC(═O)NH(CH₂)ₘ—**; —C(═O)(CH₂)ₘC(R¹²)₂—**: —C(═O)((CH₂)ₘO)ₙ(CH₂)ₘNHC(═O)X₅ C(═O)(CH₂)ₘ—**; —C(═O)((CH₂)ₘO)ₙ(CH₂)ₘNH C(═O)X₅ C(═O)(CH₂)ₘNHC(═O)(CH₂)ₘ—**; —C(═O)((CH₂)ₘO)ₙ(CH₂)ₘNH C(═O)X₅ C(═O)(CH₂)ₘX_{c}(CH₂)ₘ—**; —C(═O)((CH₂)ₘO)ₙ(CH₂)ₘNH C(═O)X₅C(═O)((CH₂)ₘO)ₙ(CH₂)ₘ—**; —C(═O)((CH₂)ₘO)ₙ(CH₂)ₘNHC(═O)X₅C(═O)((CH₂)ₘO)ₙ(CH₂)ₘNHC(═O)(CH₂)ₘ—**; —C(═O)((CH₂)ₘO)(CH₂)ₘNHC(═O)X₅C(═O)((CH₂)ₘO)ₙ(CH₂)ₘNHC(═O)(CH₂)ₘX_{c}(CH₂)ₘ—**; —C(═O)((CH₂)ₘO)(CH₂)ₘNHC(═O))X₅C(═O)(CH₂)ₘO)ₙ (CH₂)ₘX_{c}(CH₂)ₘ—**; —C(═O)(CH₂)ₘO)ₙ (CH₂)ₘNHC(═O)X₅ C(═O)(CH₂)ₘNHC(═O)(CH₂)ₘO)ₙ (CH₂)ₘ—**; —C(═O)((CH₂)ₘO)ₙ(CH₂)ₘNHC(═O)X₅C(═O)(CH₂)ₘNHC(═O)((CH₂)ₘO)ₙ(CH₂)ₘX_{c}(CH₂))ₘ—**; —C(═O)((CH₂)ₘO)ₙ(CH₂)ₘNHC(═O)X₅(CH₂)ₘX_{c}(CH₂)ₘ—**; —C(═O)(CH₂)ₘO)ₙ (CH₂)ₘNHC(═O)X₅((CH₂)ₘO)ₙ(CH₂)ₘ—**; —C(═O)(CH₂)ₘO)ₙ (CH₂)ₘNHC(═O)X₅(CH₂)ₘO)ₙ (CH₂)ₘNHC(═O)(CH₂)ₘ—**; C(═O)((CH₂)ₘO)ₙ(CH₂)ₘNHC(═O)X((CH₂)ₘO)ₙ(CH₂)ₘNHC(═O)(CH₂)ₘX_{c}(CH₂)ₘ—**; —C(═O)(CH₂)ₘO)ₙ (CH₂)ₘNHC(═O)X₅(CH₂)ₘO)ₙ (CH₂)ₘX_{c}(CH₂)ₘ—**; C(═O)((CH₂)ₘO)ₙ(CH₂)ₘNHC(═O)X₅(CH₂)ₘNH((CH₂)ₘO)ₙ(CH₂)ₘ—**; —C(═O)((CH₂)ₘO)ₙ(CH₂)ₘNHC(═O)XC(═O)(CH₂)ₘNH((CH₂)ₘO)ₙ(CH₂)ₘX_{c}(CH₂)ₘ—**; C(═O)((CH₂)ₘO)ₙ(CH₂)ₘNHC(═O)X₅(CH₂)ₘ—**; —C(═O)(CH₂)ₘC(═O)NH(CH₂)ₘ—**; —C(═O)XₐX_{b} C(═O)(CH₂)ₘNHC(═O)(CH₂)ₘ—**; —C(═O)XₐX_{b}O(═O)(CH₂)ₘX_{c}(CH₂)ₘ—**; —C(═O)XₐX_{b}C(═O)(CH₂)ₘO)ₙ(CH₂)ₘ—**; —C(═O)XₐX_{b}C(═O)((CH₂)ₘO)ₙ(CH₂)ₘNHC(═O)(CH₂)ₘ—**; —C(═O)XₐX_{b} C(═O)(CH₂)ₘO)ₙ(CH₂)ₘNHC(═O)(CH₂)ₘX_{c}(CH₂)ₘ—**; —C(═O)XₐX_{b} C(═O)(CH₂)ₘO)ₙ (CH₂)ₘX_{c}(CH₂)ₘ—**; —C(═O)XₐX_{b} C(═O)(CH₂)ₘNHC(═O)((CH₂)ₘO)ₙ(CH₂)ₘ—**; —C(═O)XₐX_{b}C(═O)(CH₂)ₘ, NHC(═O)(CH₂)ₘO)ₙ(CH₂)ₘX_{c}(CH₂)ₘ—**; —C(═O)XₐX_{b}(CH₂)ₘX_{c}(CH₂)ₘ—**; C(═O)XₐX_{b} ((CH₂)ₘO)ₙ (CH₂) m-**; —C(═O)XₐX_{b}((CH₂)ₘO)ₙ(CH₂)ₘNHC(═O)(CH₂))ₘ—**; —C(═O)XₐX_{b}(CH₂)ₘO)ₙ(CH₂)ₘNHC(═O)(CH₂)ₘX_{c}(CH₂)ₘ—**; —C(═O)XₐX_{b}(CH₂)ₘO)ₙ (CH₂)ₘX_{c}(CH₂)ₘ—**; —C(═O)XₐX_{b}(CH₂)ₘNH((CH₂)ₘO)ₙ(CH₂)ₘ—**; —C(═O)XₐX_{b} C(═O)(CH₂)ₘNH((CH₂)ₘO)ₙ(CH₂)ₘX_{c}(CH₂)ₘ—**; —C(═O)XₐC(═O)NH(CH₂)ₘX₅(CH₂)ₘ—**; —C(═O)XₐX_{b}(CH₂)ₘ—**; —C(═O)X1C(═O)(CH₂)ₘNHC(═O)((CH₂)ₘO)ₙ(CH₂)ₘ—**; —C(═O)NH(CH₂)ₘ—**; —C(═O)NH(CH₂)ₘX_{c}(CH₂)ₘ—**; —C(═O)NH(CH₂)ₘNHC(═O)XₐX_{b} C(═O)(CH₂)ₘ—**; —C(═O)NH(CH₂)ₘNHC(═O)Q(CH₂)ₘ—**; —C(═O)NH(CH₂)ₘNHC(═O)XₐX_{b}—**; —C(═O)NH(CH₂)ₘNHC(═O)X₅—**; —C(═O)NH(CH₂)ₘNHC(═O)(CH₂)ₘX₅(CH₂)ₘ—**; —C(═O)NHC(═O)(CH₂)ₘ NHC(═O)XₐX_{b} C(═O)(CH₂)ₘ—**; C(═O)NH(CH₂)ₘNHC(═O)(CH₂)ₘ—**; —C(═O)NH(CH₂)ₘNHC(═O)(CH₂)ₘO(CH₂)ₘ—**; —C(═O)NH(CH₂)ₘNHC(═O)XₐX_{b} C(═O)(CH₂)ₘO(CH₂)ₘ—**; —C(═O)NH(CH₂)ₘNHC(═O)XₐX_{b} C(═O)(CH₂)ₘO(CH₂)ₘC(═O)—**; —C(═O)NH(CH₂)ₘNHC(═O)X₄C(═O)NH(CH₂)ₘNHC(═O)(CH₂)ₘO(CH₂)ₘ—**: —C(═O)NH(CH₂)ₘNHC(═O)X₅C(═O)(CH₂)ₘ—**; —C(═O)NH(CH₂)ₘNH C(═O)X₅ C(═O)(CH₂)ₘNHC(═O)(CH₂)ₘ—**; —C(═O)NH(CH₂)ₘNHC(═O)X₅C(═O)(CH₂)ₘX_{C}(CH₂)ₘ—**; —C(═O)NH(CH₂)ₘNH C(═O)X₅C(═O)(CH₂)ₘO)ₙ (CH₂)ₘ—**; —C(═O)NH(CH₂)ₘNH C(═O)X₅ C(═O)(CH₂)ₘO)ₙ(CH₂)ₘNHC(═O)(CH₂)ₘ—**; —C(═O)NH(CH₂)ₘNH C(═O)X₅ C(═O)(CH₂)ₘO)ₙ (CH₂)ₘNHC(═O)(CH₂)ₘX_{C}(CH₂)ₘ—**; —C(═O)NH(CH₂)ₘNHC(═O)X₅C(═O)((CH₂)ₘO)ₙ(CH₂)ₘX_{C}(CH₂)ₘ—**; C(═O)NH(CH₂)ₘNHC(═O)X₅C(═O)(CH₂)ₘNHC(═O)(CH₂)ₘO)ₙ (CH₂)ₘ—**; —C(═O)NH(CH₂)ₘNH C(═O)X₅ C(═O)(CH₂)ₘNHC(═O)(CH₂)ₘO)ₙ (CH₂)ₘX_{c}(CH₂)ₘ—**; —C(═O)NH(CH₂)ₘNHC(═O)X₅(CH₂)ₘX_{C}(CH₂)ₘ—**; —C(═O)NH(CH₂)ₘNHC(═O)X₅((CH₂)ₘO)ₙ(CH₂)ₘ—**; —C(═O)NH(CH₂)ₘNHC(═O)X₅(CH₂)ₘO)ₙ(CH₂)ₘNHC(═O)(CH₂)ₘ—**; —C(═O)NH(CH₂)ₘNHC(═O)X₅((CH₂)ₘO)ₙ(CH₂)ₘNHC(═O)(CH₂)ₘX_{C}(CH₂)ₘ—**; C(═O)NH(CH₂)ₘNHC(═O)X₅((CH₂)ₘO)ₙ(CH₂)mXC(CH₂)ₘ—**; —C(═O)NH(CH₂)ₘNHC(═O)X₅(CH₂)ₘNH(CH₂)ₘO)ₙ (CH₂)ₘ—**; —C(═O)NH(CH₂)ₘNH C(═O)X₅ C(═O)(CH₂)ₘNH((CH₂)ₘO)ₙ(CH₂)mXC(CH₂)ₘ—**; —C(═O)NH(CH₂)ₘNHC(═O)X₅(CH₂)ₘ—**; —C(═O)X1C(═O)NH(CH₂)ₘNHC(═O)(CH₂)ₘ—**; —C(═O)X1C(═O)NH(CH₂)ₘX_{c}(CH₂)ₘ—**; —C(═O)NH(CH₂)ₘNHC(═O)(CH₂)ₘX_{c}(CH₂)ₘ—**; —C(═O)NH(CH₂)ₘNHC(═O)—**; —C(═O)NH((CH₂)ₘO)ₙ(CH₂)ₘX_{c}(CH₂)ₘ—** or —C(═O)X1C(═O)(CH₂)ₘNHC(═O)(CH₂)ₘ—**;
Xₐ is

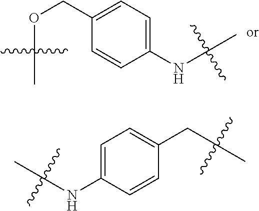 [SMILES: *Nc1ccc(CO*)cc1 ; *Cc1ccc(N*)cc1]

where the * of Xₐ indicates the point of attachment to X_{b}; X_{b} is selected from

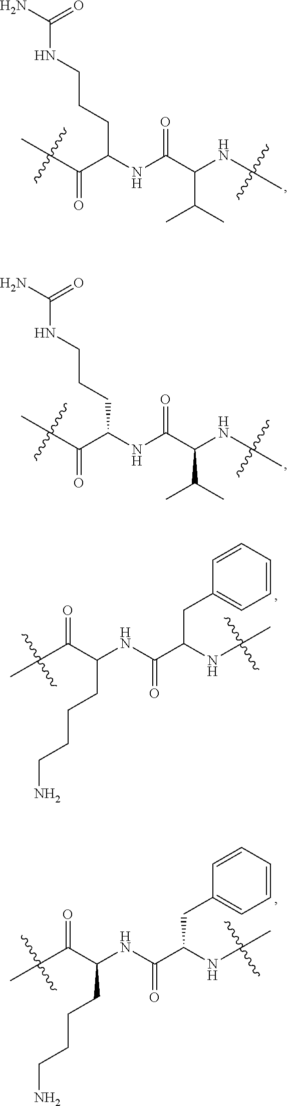 [SMILES: *NC(C(=O)NC(CCCNC(N)=O)C(*)=O)C(C)C ; *N[C@H](C(=O)N[C@@H](CCCNC(N)=O)C(*)=O)C(C)C ; *NC(Cc1ccccc1)C(=O)NC(CCCCN)C(*)=O ; *N[C@@H](Cc1ccccc1)C(=O)N[C@@H](CCCCN)C(*)=O]

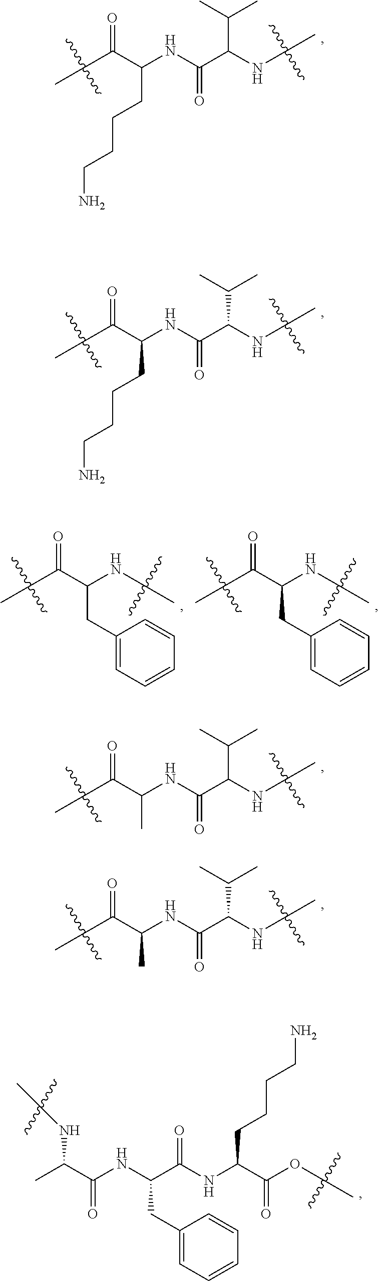

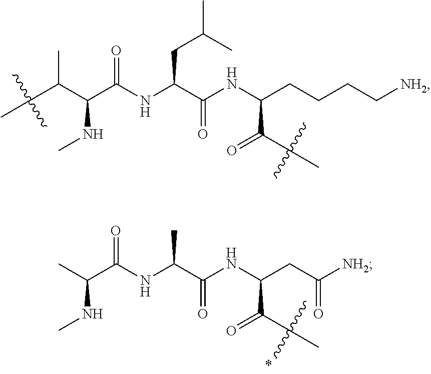 [SMILES: *C(=O)[C@H](CCCCN)NC(=O)[C@H](CC(C)C)NC(=O)[C@@H](NC)C(*)C ; *C(=O)[C@H](CC(N)=O)NC(=O)[C@H](C)NC(=O)[C@H](C)NC]

where the * of X_{b}indicates the point of attachment to Xₐ, —NH—, NHNH—, —NHO— or —NHN═CR¹²(CH₂)ₙ—;

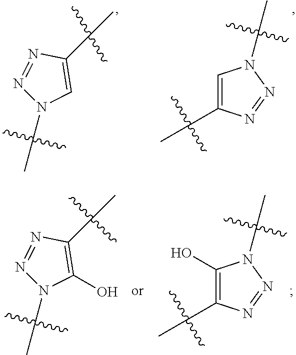 [SMILES: *c1cn(*)nn1 ; *c1cn(*)nn1 ; *c1nnn(*)c1O ; "or" ; *c1nnn(*)c1O ; ";"]

X₄ is —O(CH₂)ₙSSC(R¹²)₂(CH₂)ₙ—or —(CH₂)ₙC(R¹²)₂SS(CH₂)ₙO—;
X₅ is

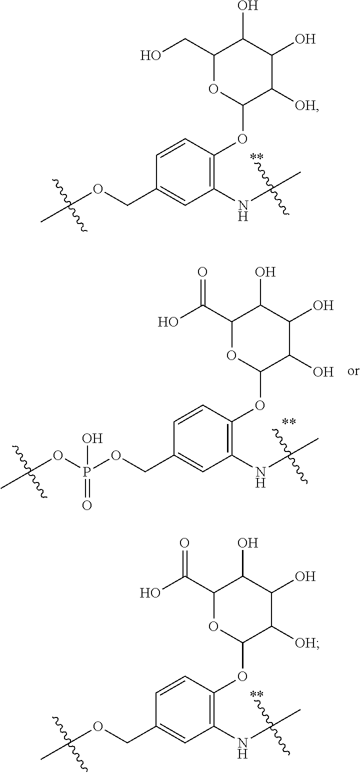 [SMILES: *Nc1cc(CO*)ccc1OC1OC(CO)C(O)C(O)C1O ; *Nc1cc(COP(=O)(O)O*)ccc1OC1OC(C(=O)O)C(O)C(O)C1O ; "or" ; *Nc1cc(CO*)ccc1OC1OC(C(=O)O)C(O)C(O)C1O]

each m is independently selected from 1, 2, 3, 4, 5, 6, 7, 8, 9 and 10; and
each n is independently selected from 1, 2, 3, 4, 5, 6, 7, 8, 9, 10, 11, 12, 13, 14, 15, 16, 17 and 18.

In some embodiments, wherein the linker L¹ is selected from:

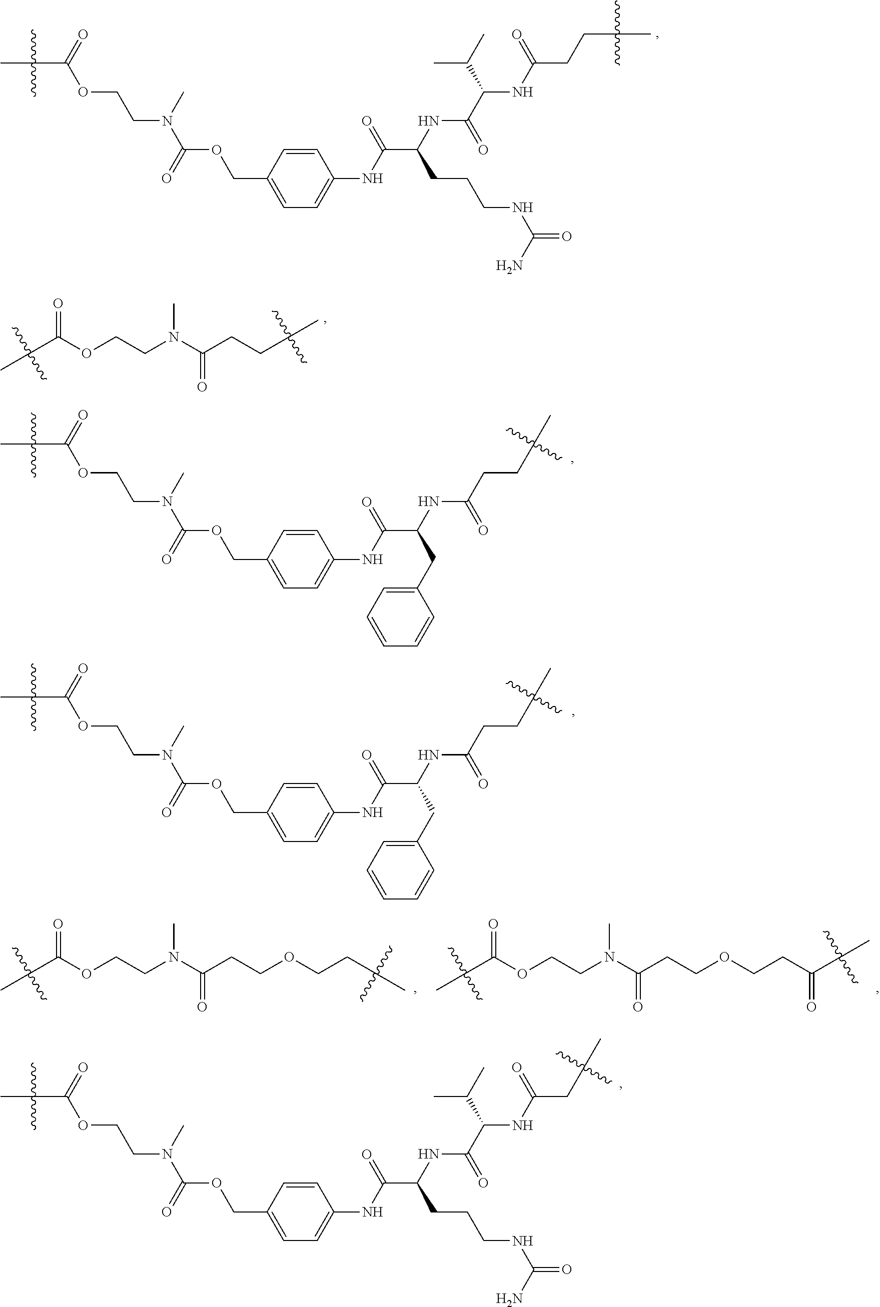

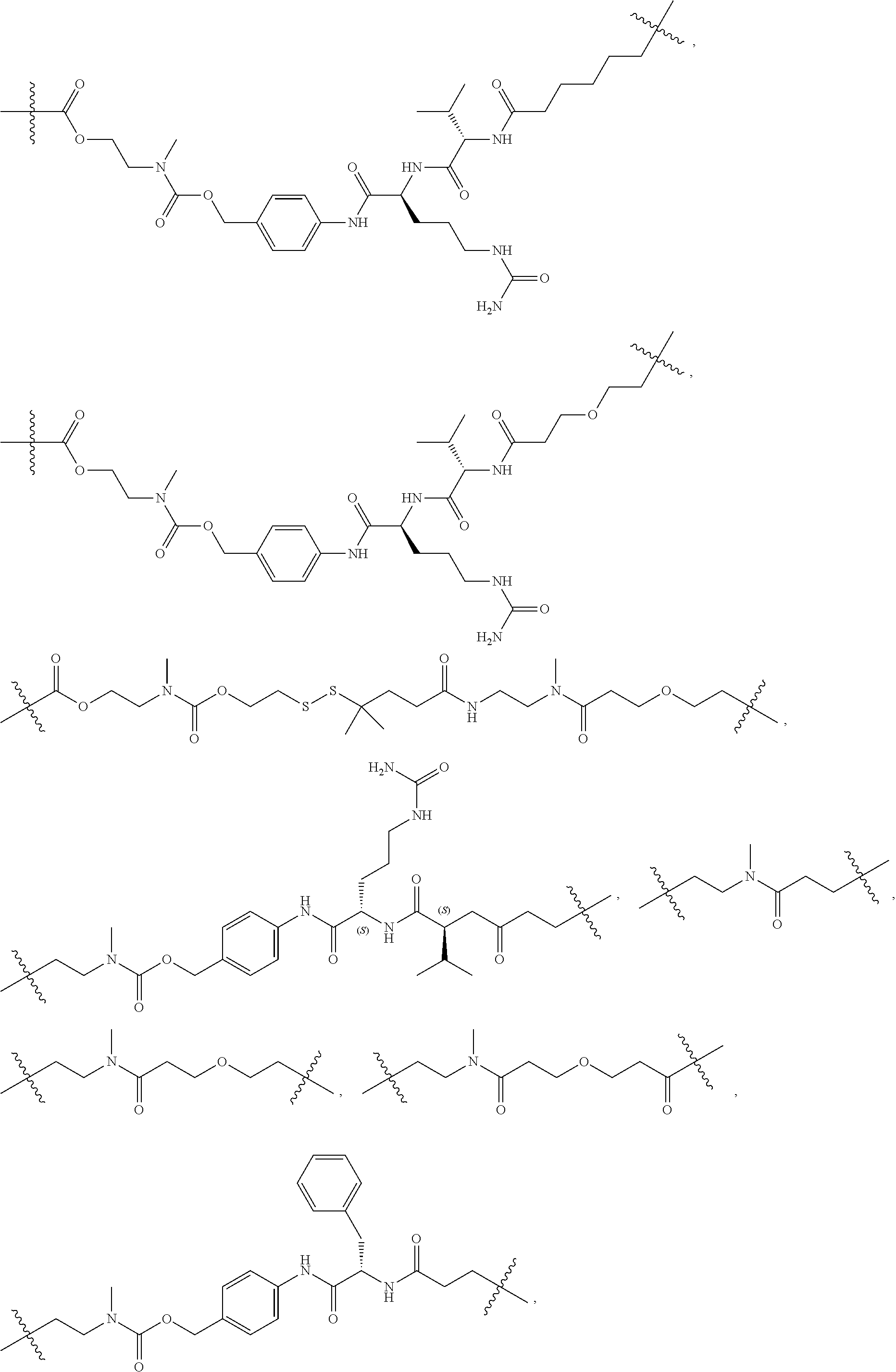

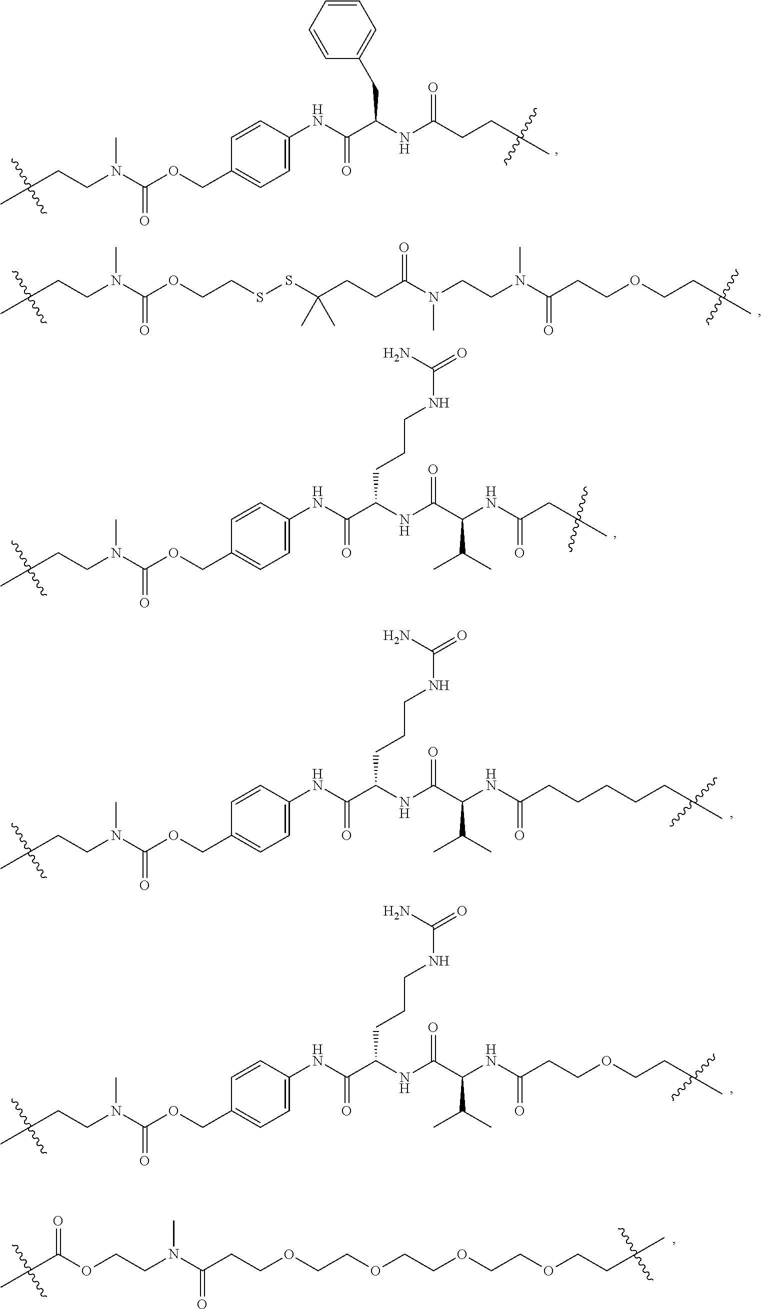

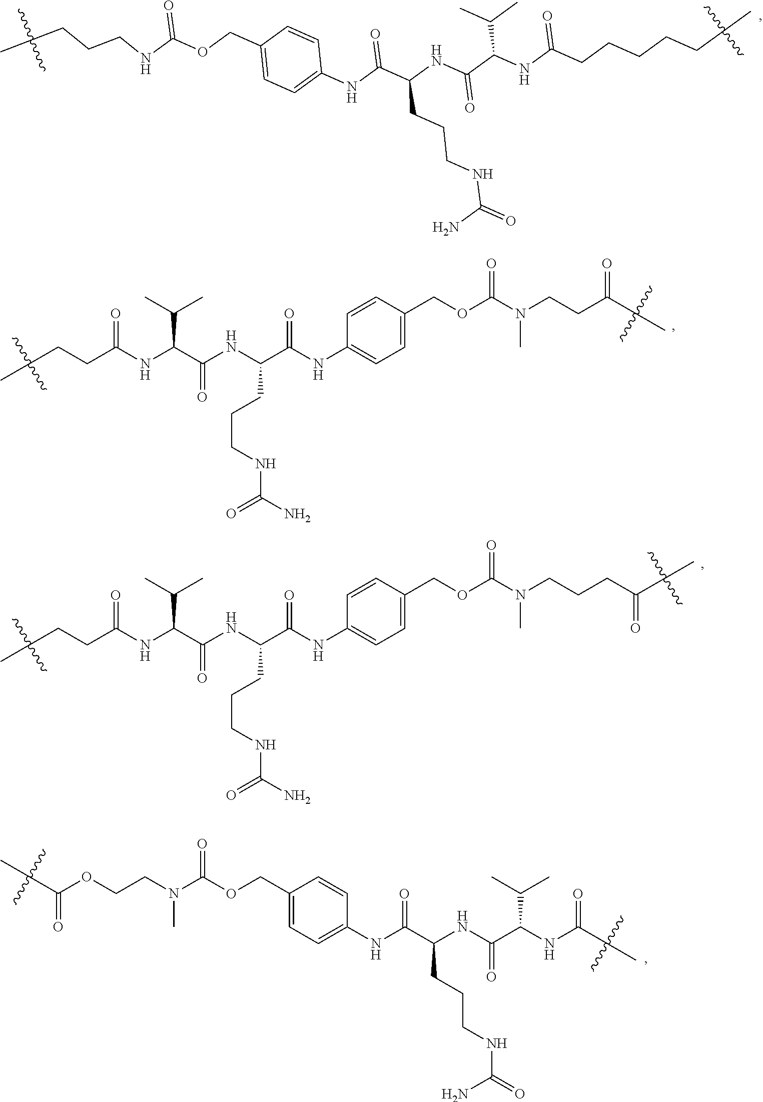 [SMILES: *CCCCCC(=O)N[C@H](C(=O)N[C@@H](CCCNC(N)=O)C(=O)Nc1ccc(COC(=O)NCCC*)cc1)C(C)C ; *CCC(=O)N[C@H](C(=O)N[C@@H](CCCNC(N)=O)C(=O)Nc1ccc(COC(=O)N(C)CCC(*)=O)cc1)C(C)C ; *CCC(=O)N[C@H](C(=O)N[C@@H](CCCNC(N)=O)C(=O)Nc1ccc(COC(=O)N(C)CCCC(*)=O)cc1)C(C)C ; *C(=O)N[C@H](C(=O)N[C@@H](CCCNC(N)=O)C(=O)Nc1ccc(COC(=O)N(C)CCOC(*)=O)cc1)C(C)C]

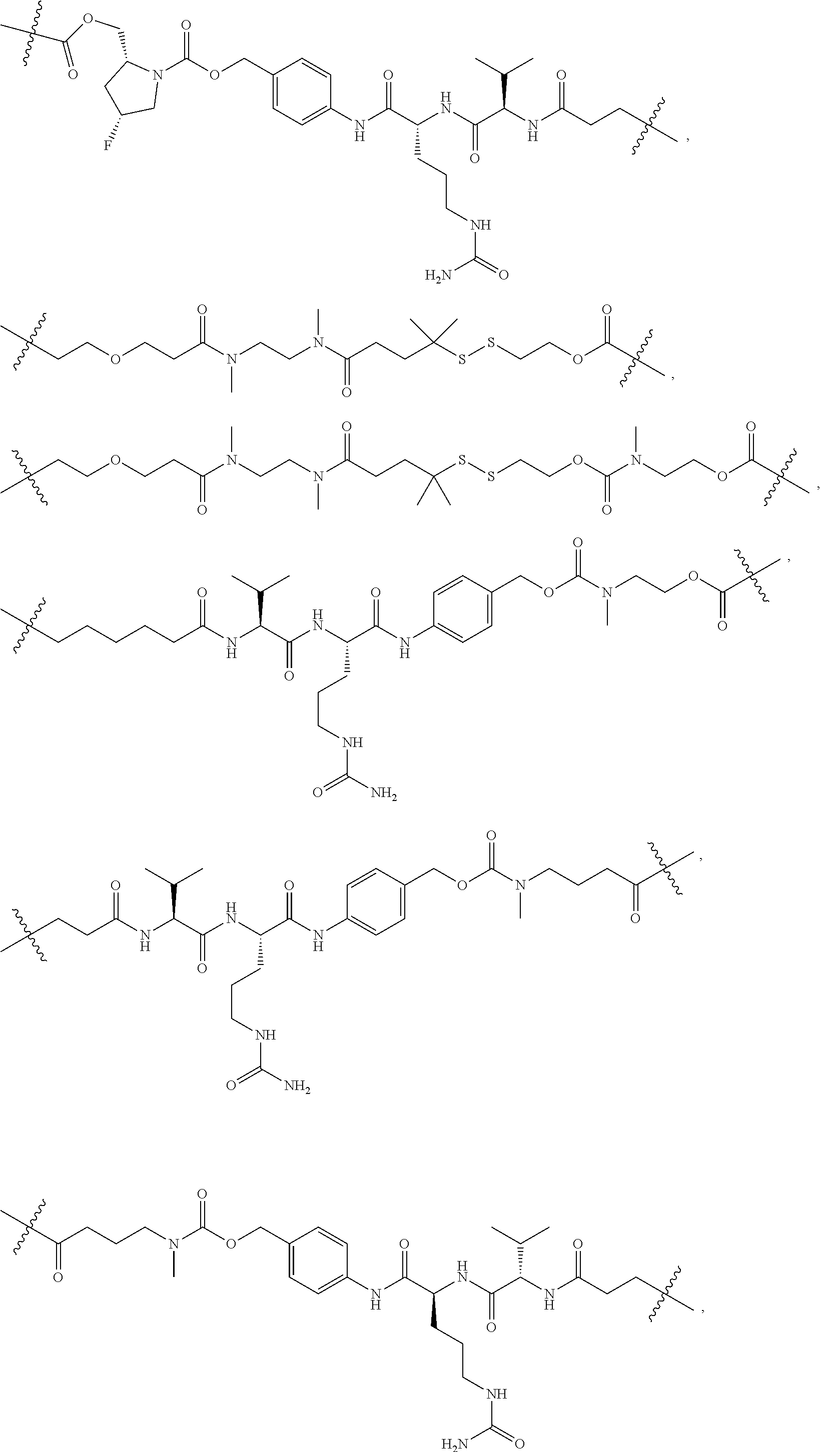

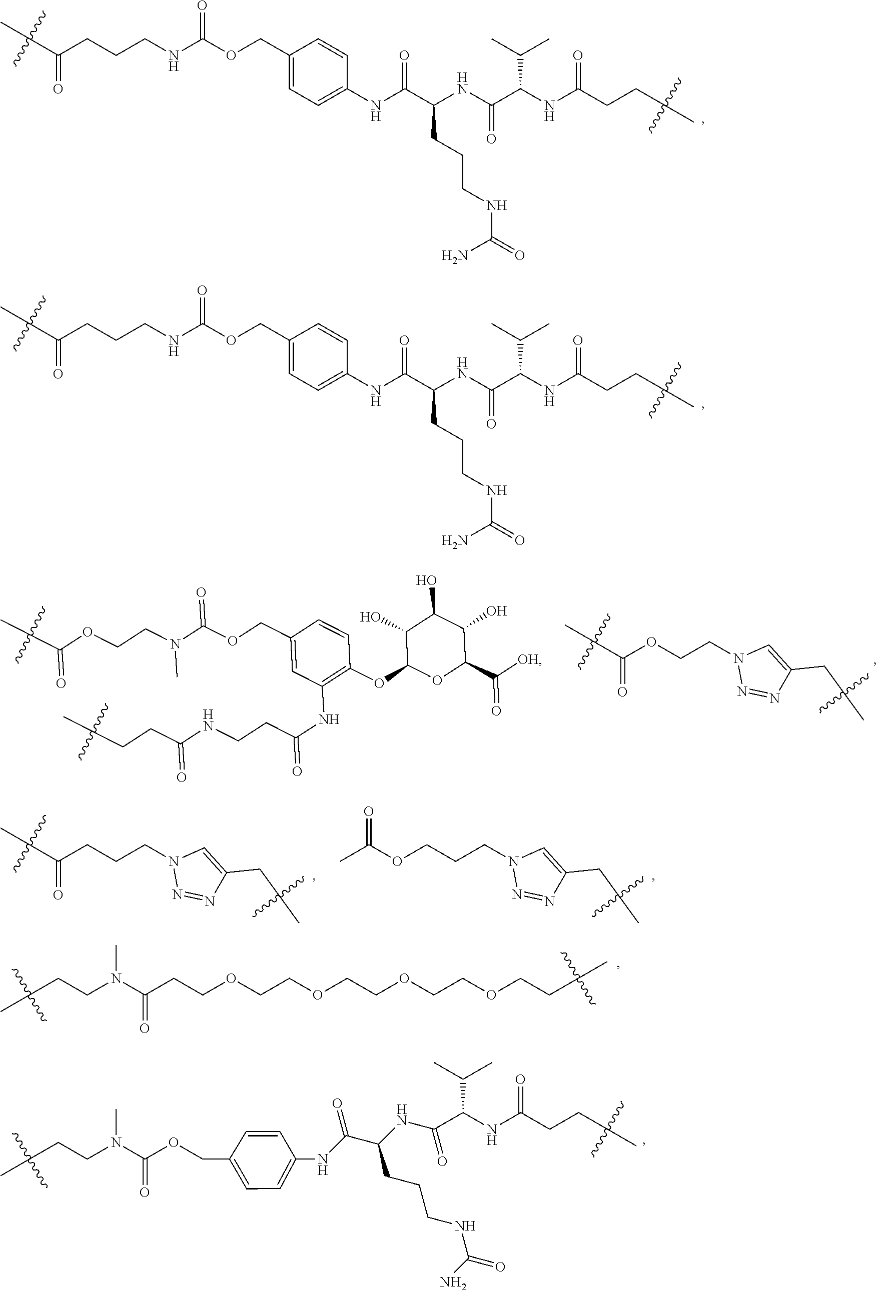

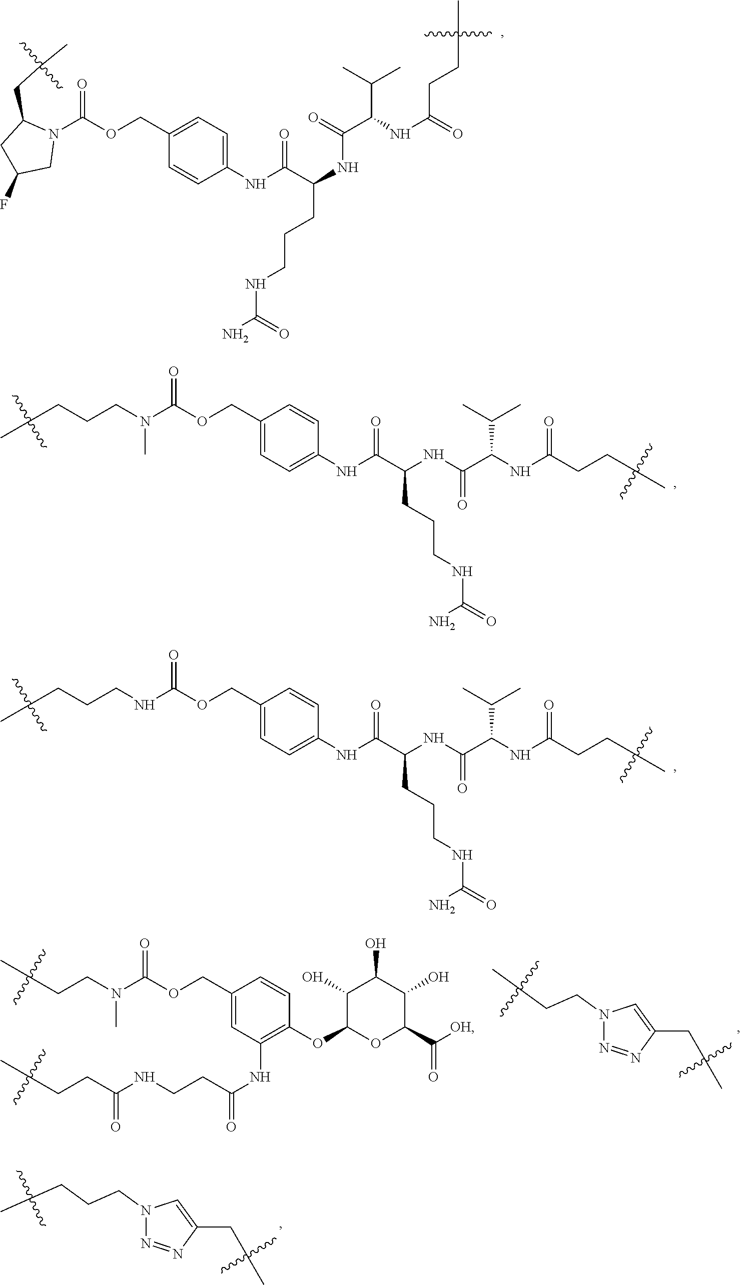 [SMILES: *CCC(=O)N[C@H](C(=O)N[C@@H](CCCNC(N)=O)C(=O)Nc1ccc(COC(=O)N2C[C@@H](F)C[C@H]2C*)cc1)C(C)C ; *CCCN(C)C(=O)OCc1ccc(NC(=O)[C@H](CCCNC(N)=O)NC(=O)[C@@H](NC(=O)CC*)C(C)C)cc1 ; *CCCNC(=O)OCc1ccc(NC(=O)[C@H](CCCNC(N)=O)NC(=O)[C@@H](NC(=O)CC*)C(C)C)cc1 ; *CCC(=O)NCCC(=O)Nc1cc(COC(=O)N(C)CC*)ccc1O[C@@H]1O[C@H](C(=O)O)[C@@H](O)[C@H](O)[C@H]1O ; *CCn1cc(C*)nn1 ; *CCCn1cc(C*)nn1]

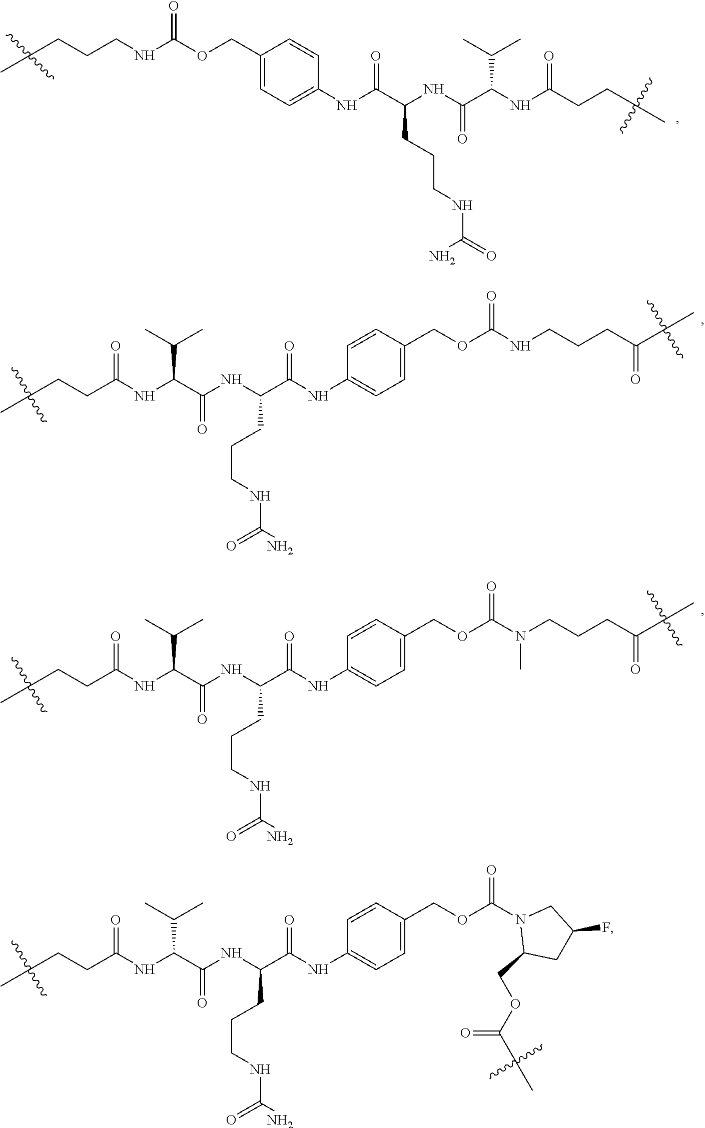

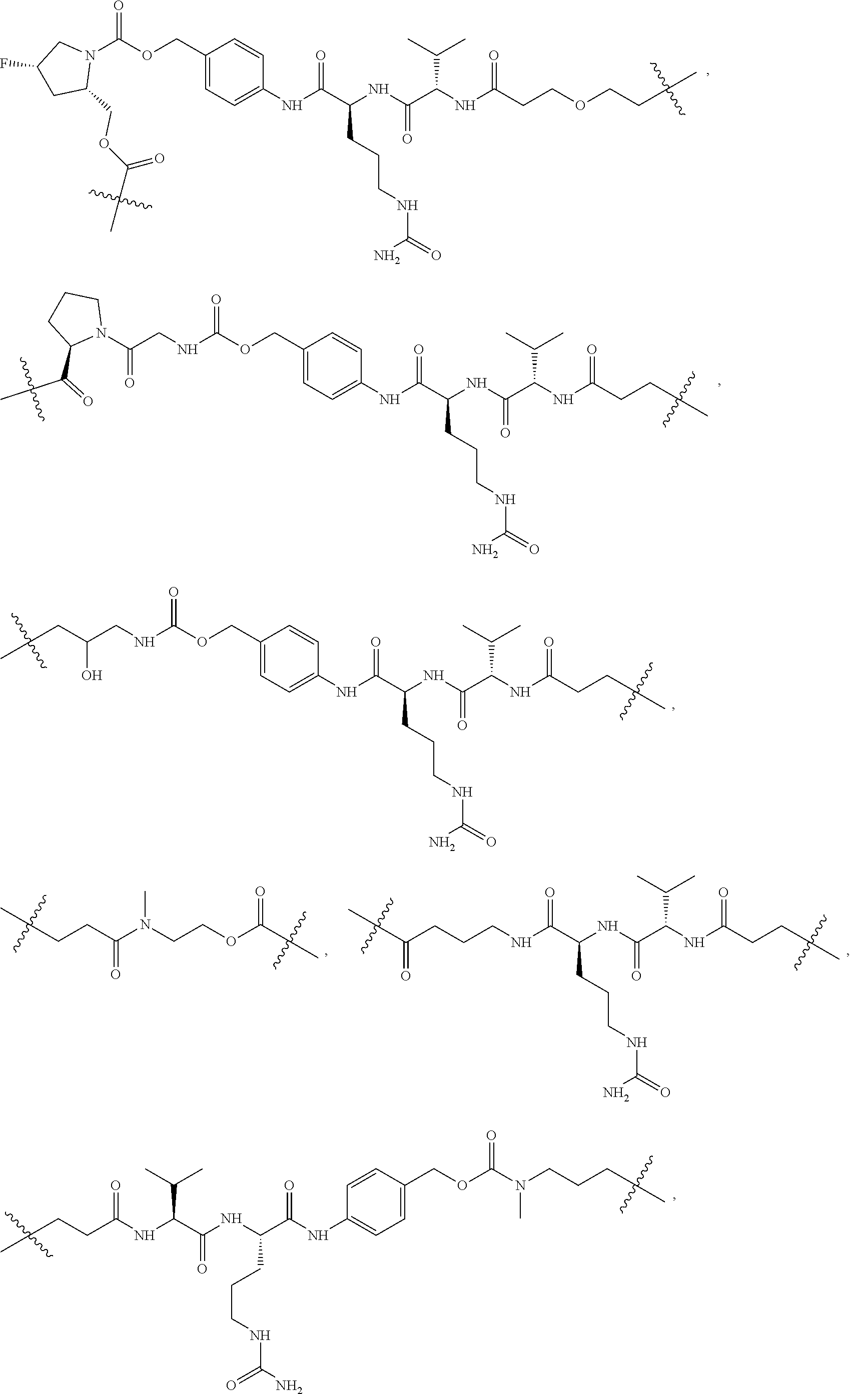 [SMILES: *CCOCCC(=O)N[C@H](C(=O)N[C@@H](CCCNC(N)=O)C(=O)Nc1ccc(COC(=O)N2C[C@@H](F)C[C@H]2COC(*)=O)cc1)C(C)C ; *CCC(=O)N[C@H](C(=O)N[C@@H](CCCNC(N)=O)C(=O)Nc1ccc(COC(=O)NCC(=O)N2CCC[C@@H]2C(*)=O)cc1)C(C)C ; *CCC(=O)N[C@H](C(=O)N[C@@H](CCCNC(N)=O)C(=O)Nc1ccc(COC(=O)NCC(O)C*)cc1)C(C)C ; *CCC(=O)N(C)CCOC(*)=O ; *CCC(=O)N[C@H](C(=O)N[C@@H](CCCNC(N)=O)C(=O)NCCCC(*)=O)C(C)C ; *CCCN(C)C(=O)OCc1ccc(NC(=O)[C@H](CCCNC(N)=O)NC(=O)[C@@H](NC(=O)CC*)C(C)C)cc1]

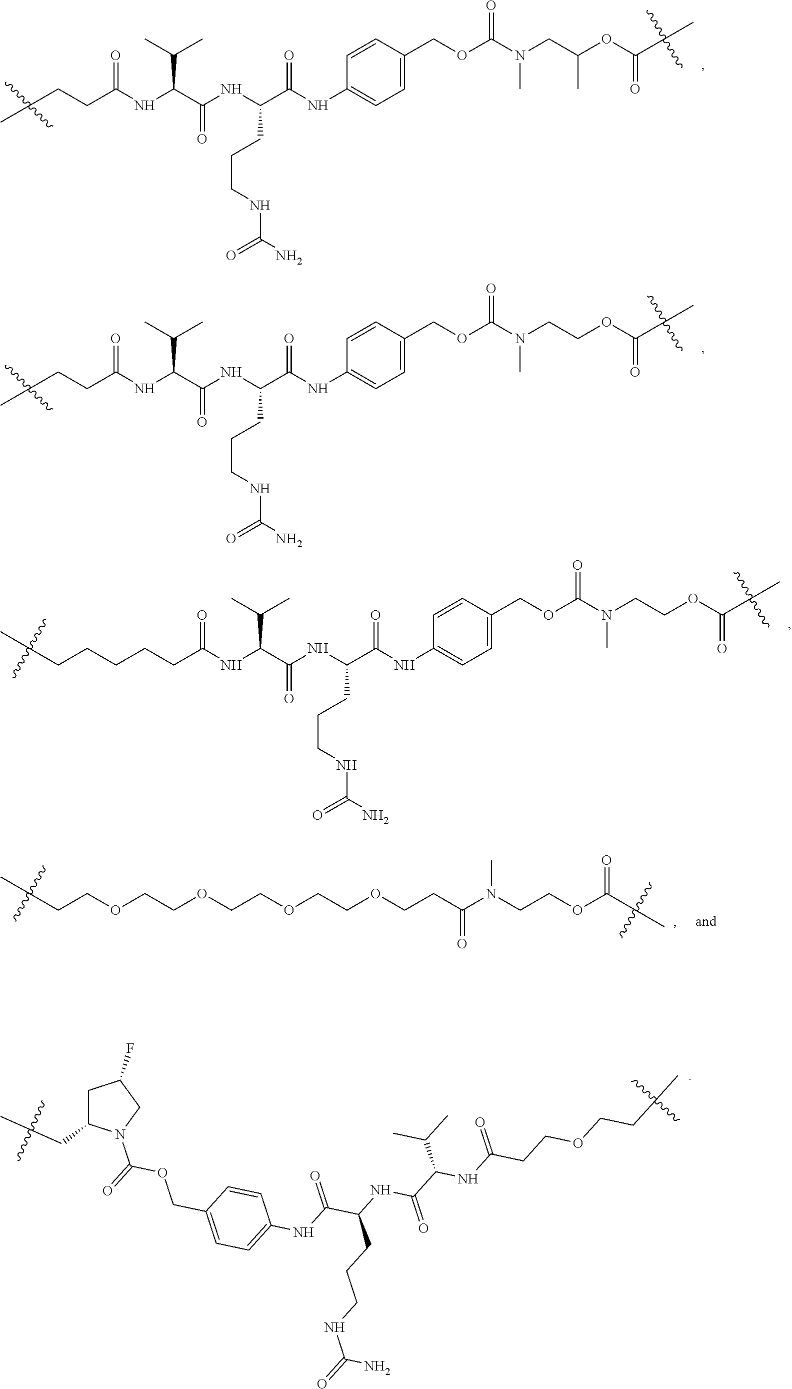 [SMILES: *CCC(=O)N[C@H](C(=O)N[C@@H](CCCNC(N)=O)C(=O)Nc1ccc(COC(=O)N(C)CC(C)OC(*)=O)cc1)C(C)C ; *CCC(=O)N[C@H](C(=O)N[C@@H](CCCNC(N)=O)C(=O)Nc1ccc(COC(=O)N(C)CCOC(*)=O)cc1)C(C)C ; *CCCCCC(=O)N[C@H](C(=O)N[C@@H](CCCNC(N)=O)C(=O)Nc1ccc(COC(=O)N(C)CCOC(*)=O)cc1)C(C)C ; *CCOCCOCCOCCOCCC(=O)N(C)CCOC(*)=O ; *CCOCCC(=O)N[C@H](C(=O)N[C@@H](CCCNC(N)=O)C(=O)Nc1ccc(COC(=O)N2C[C@@H](F)C[C@H]2C*)cc1)C(C)C]

In some embodiments, the linker L¹ is selected from the group consisting of:

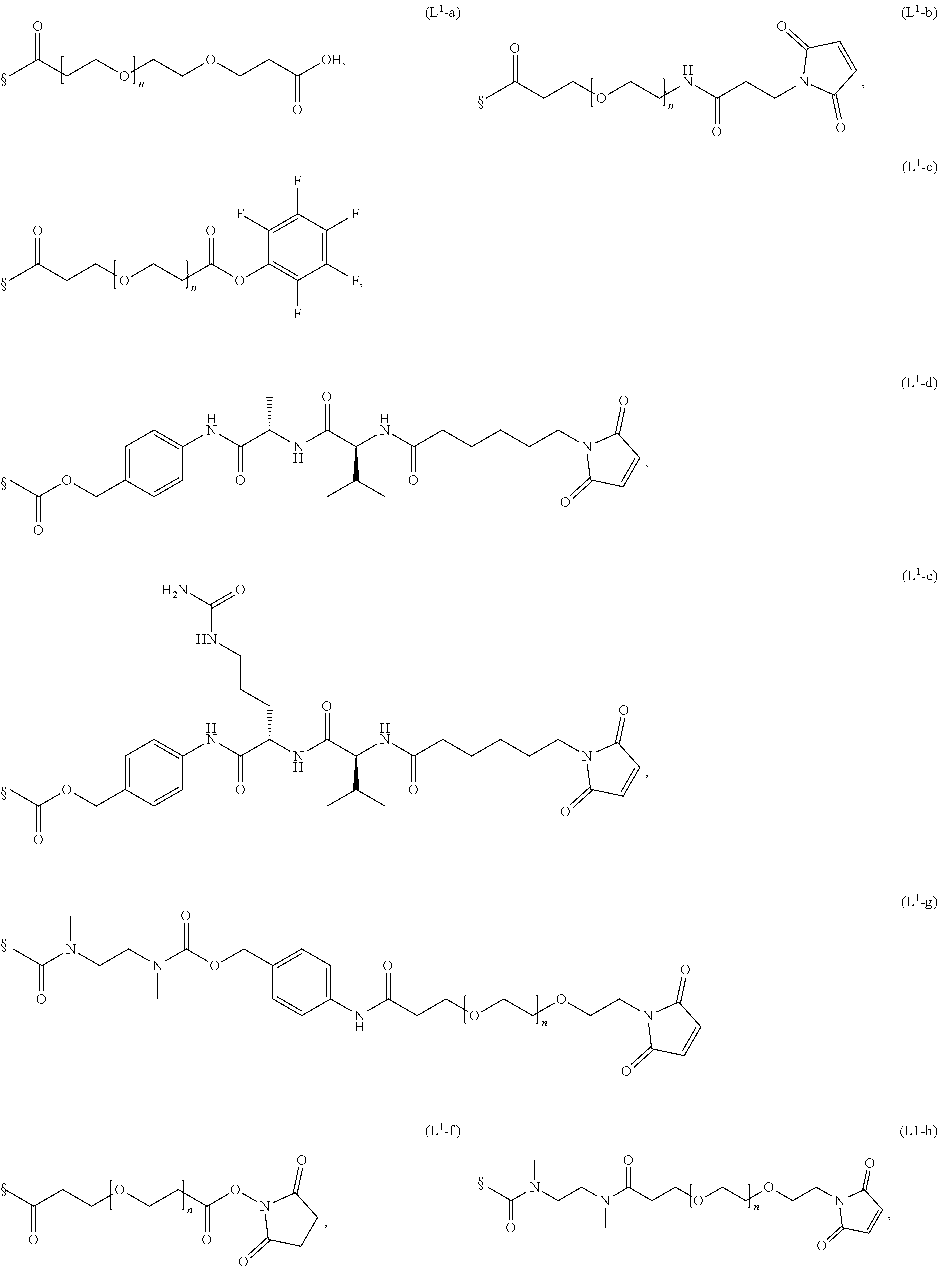

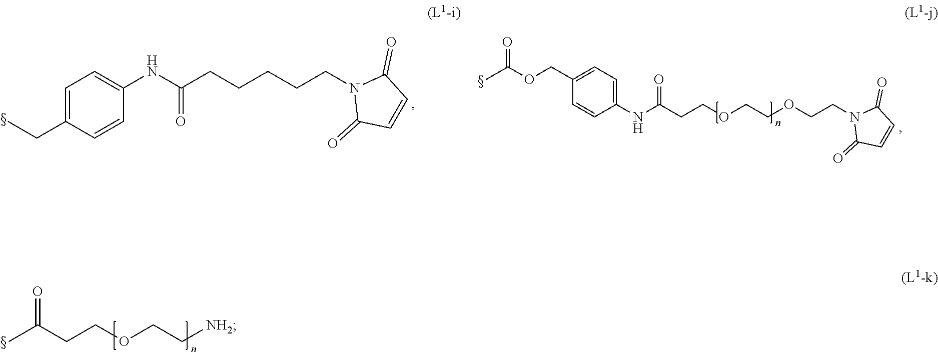

wherein § marks the connection to the compound of formula (Y-1), (Y-2), (Y-3), (A), (B), (C), I, H, III, IV or V, and wherein n is 1-12, preferably 8; “ [symbol] ” denotes attachment to the STING agonist moiety(also named D herein).

In some embodiments, the linker L¹ is selected from the group consisting of

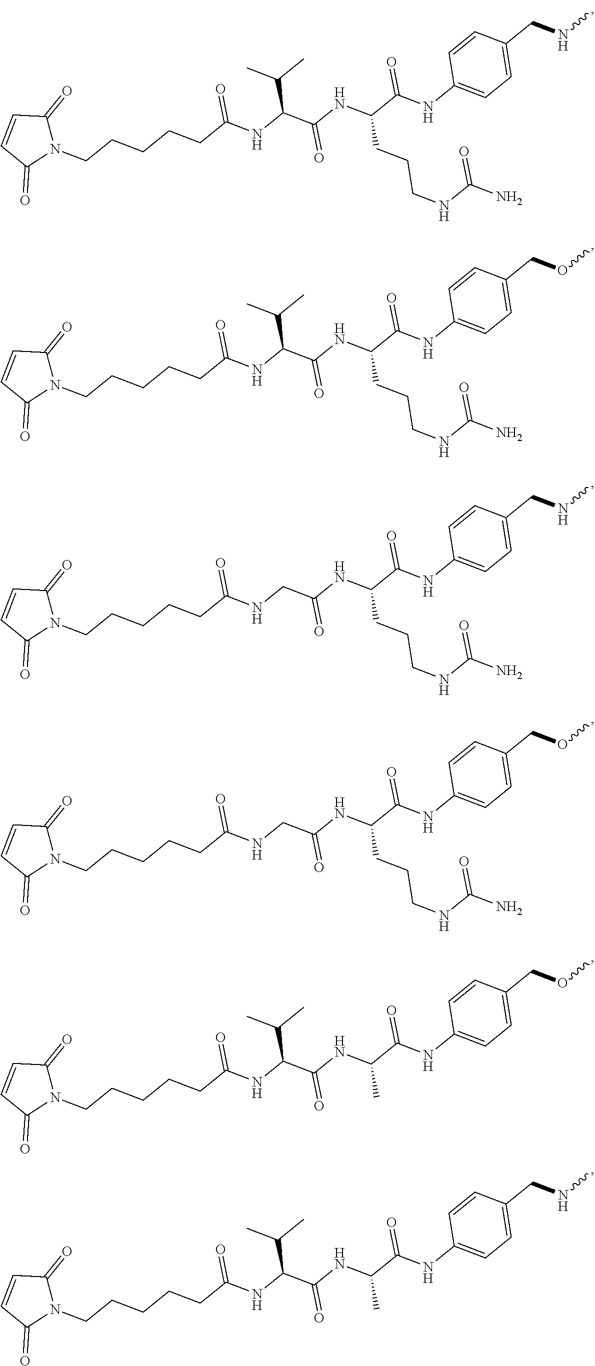

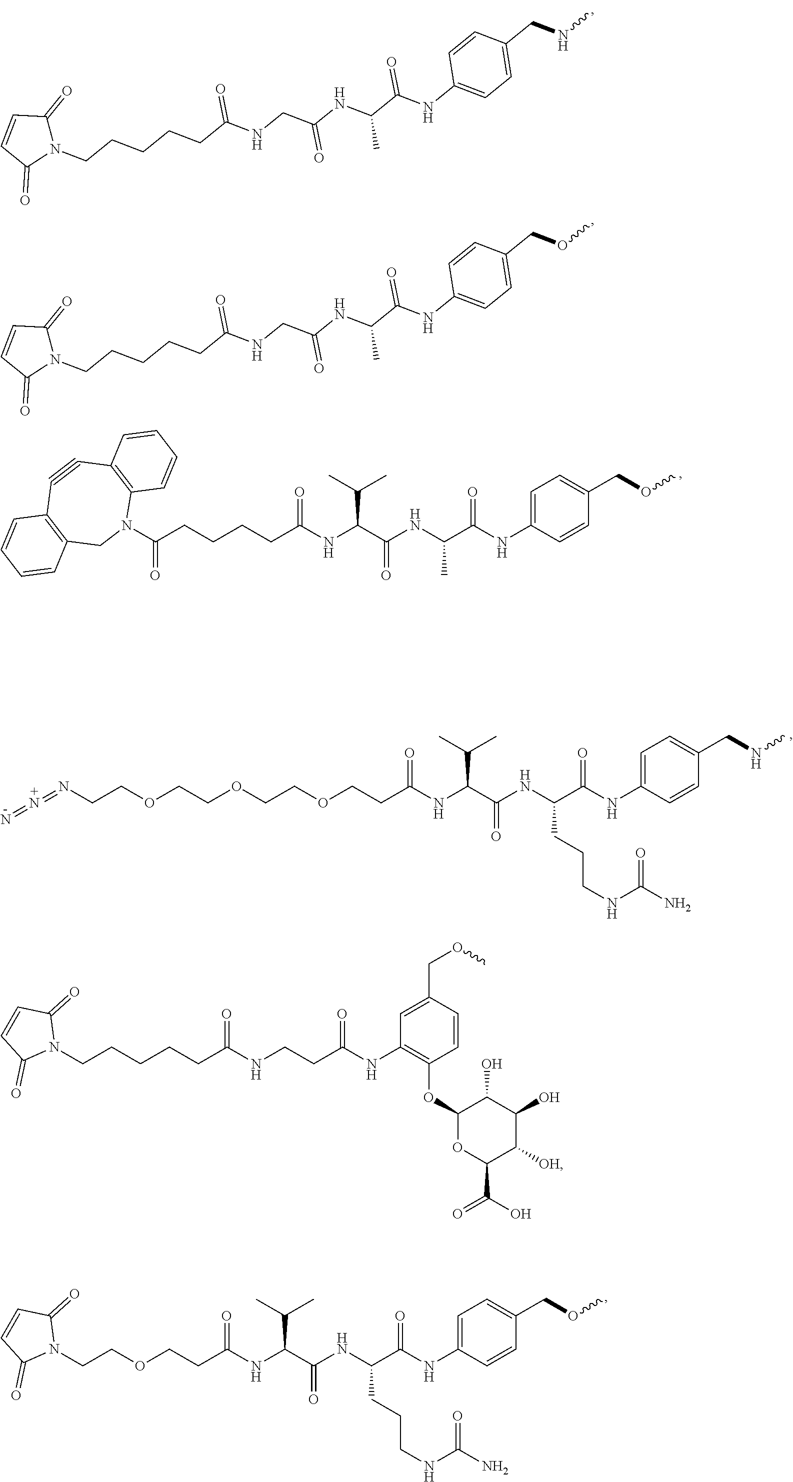

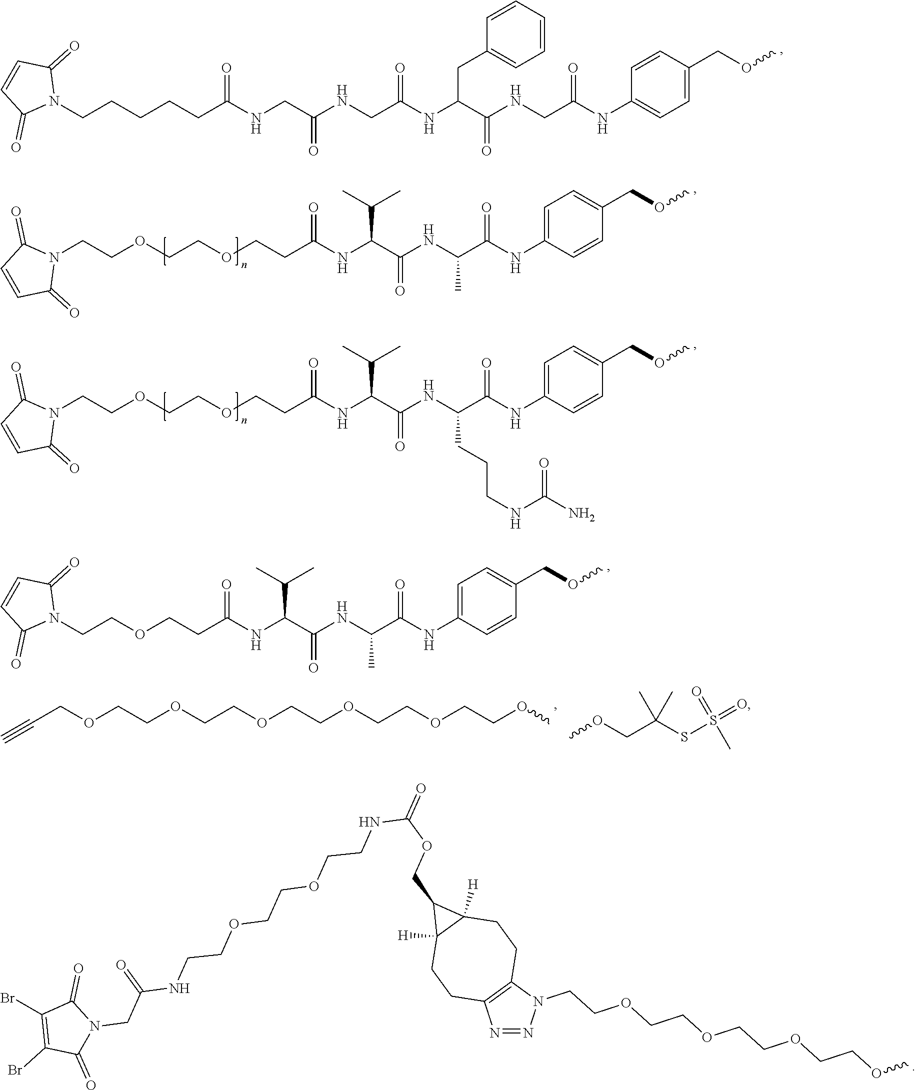

In some embodiments, the compound-linker construct of the present disclosure, is selected from the group consisting of:

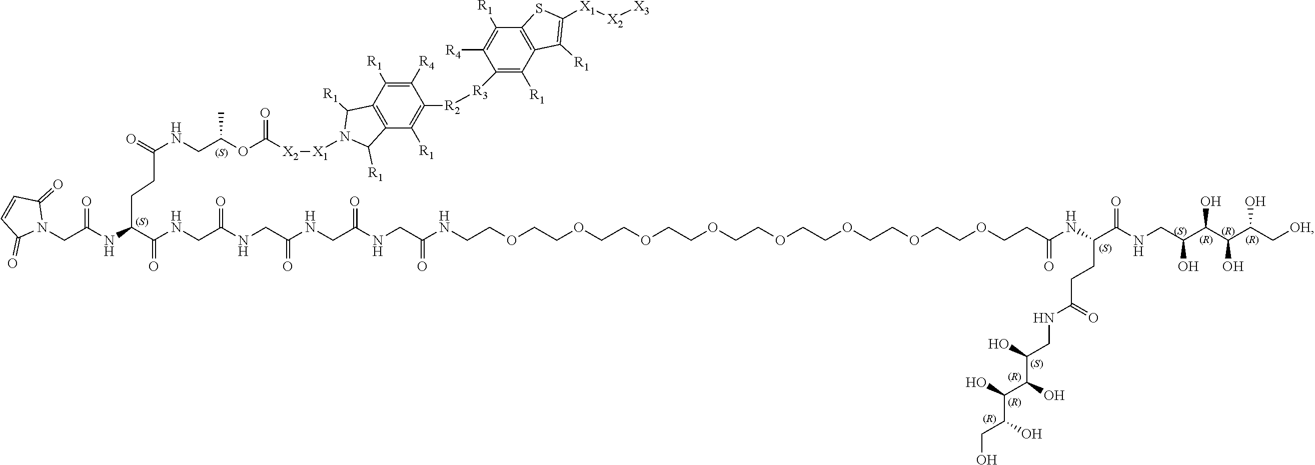

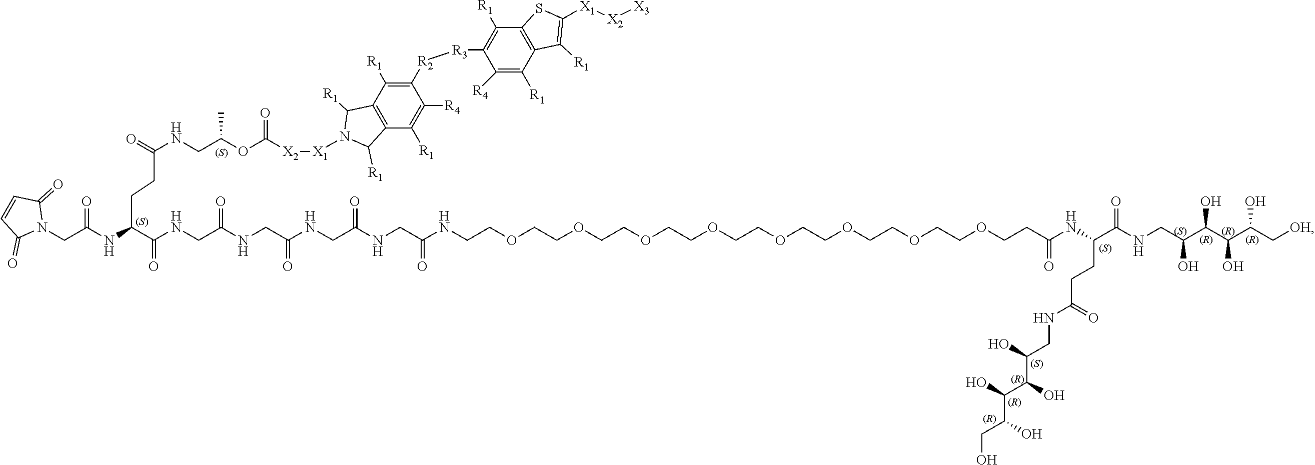

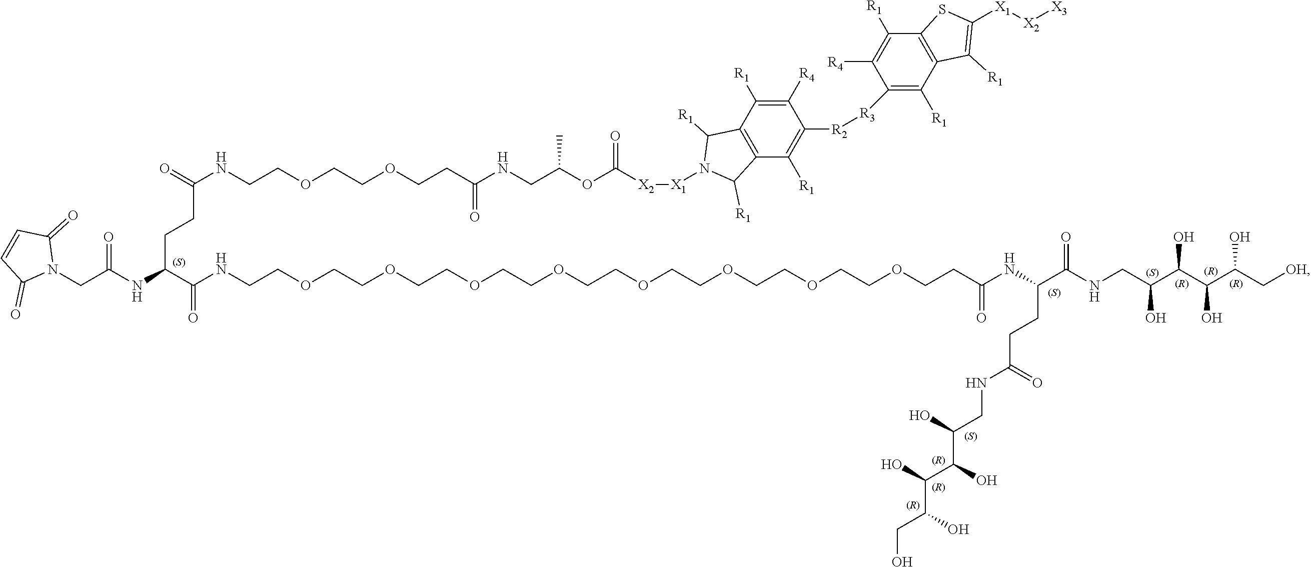

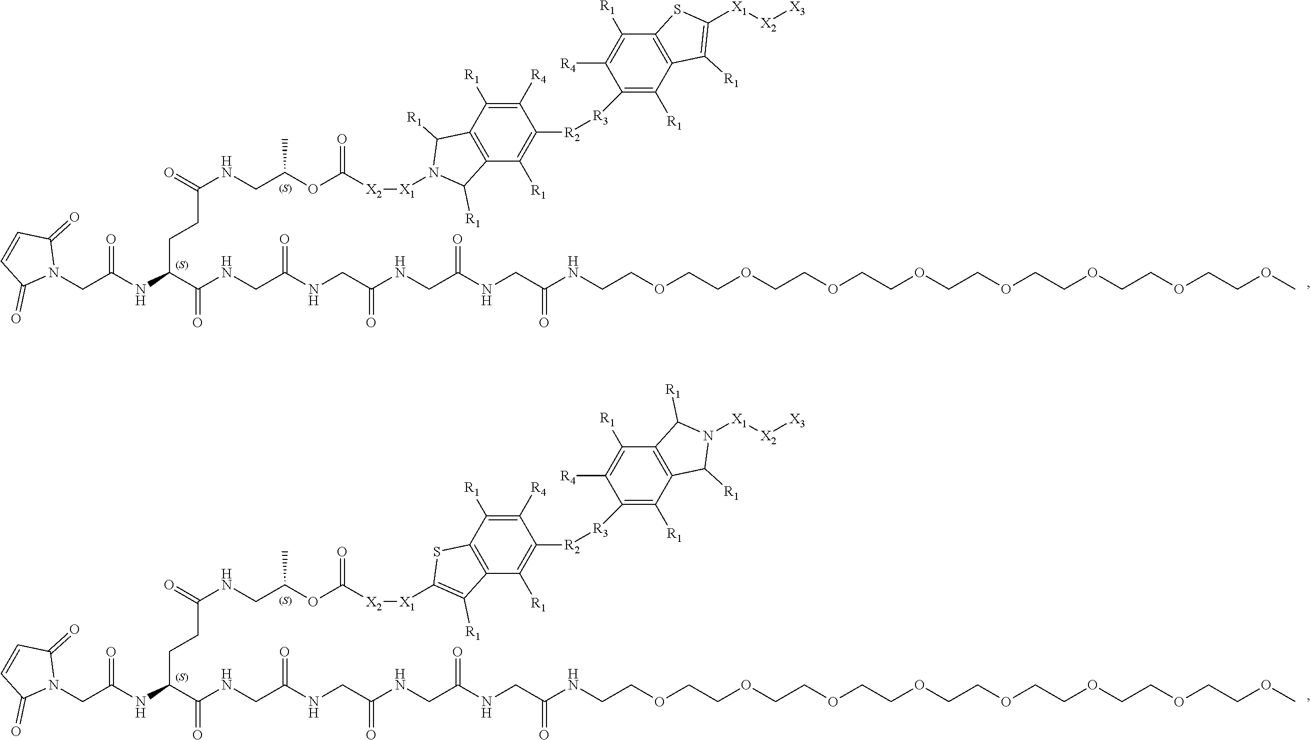

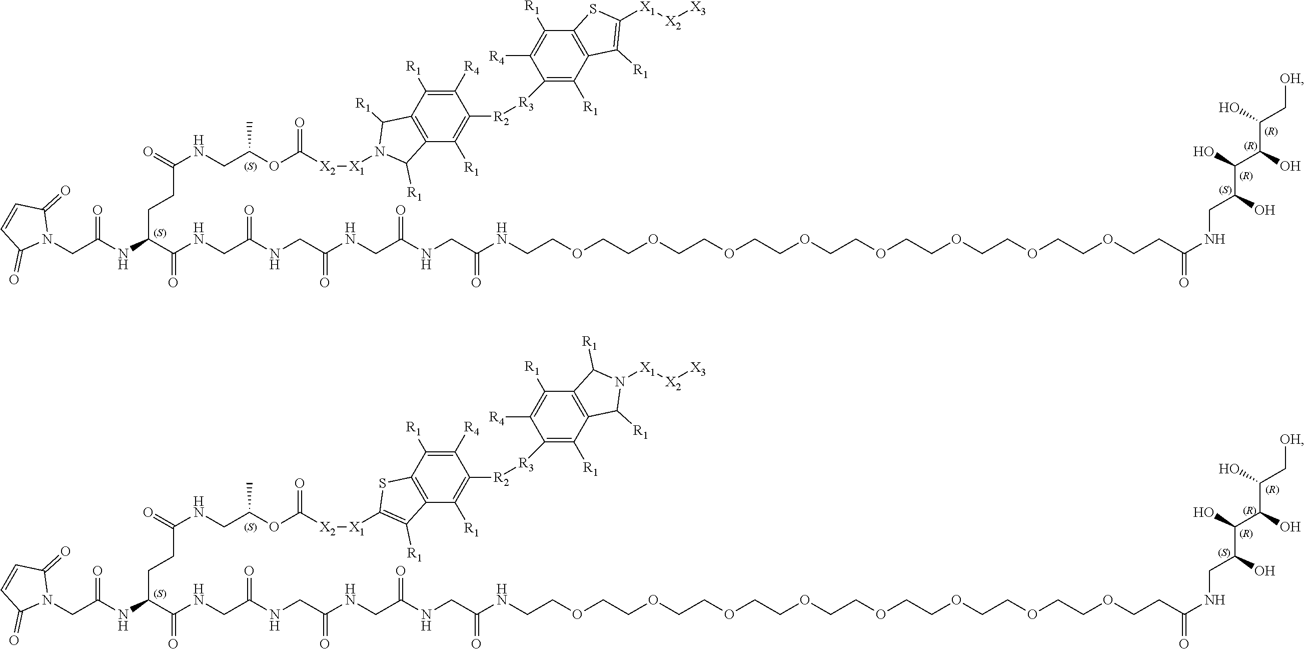

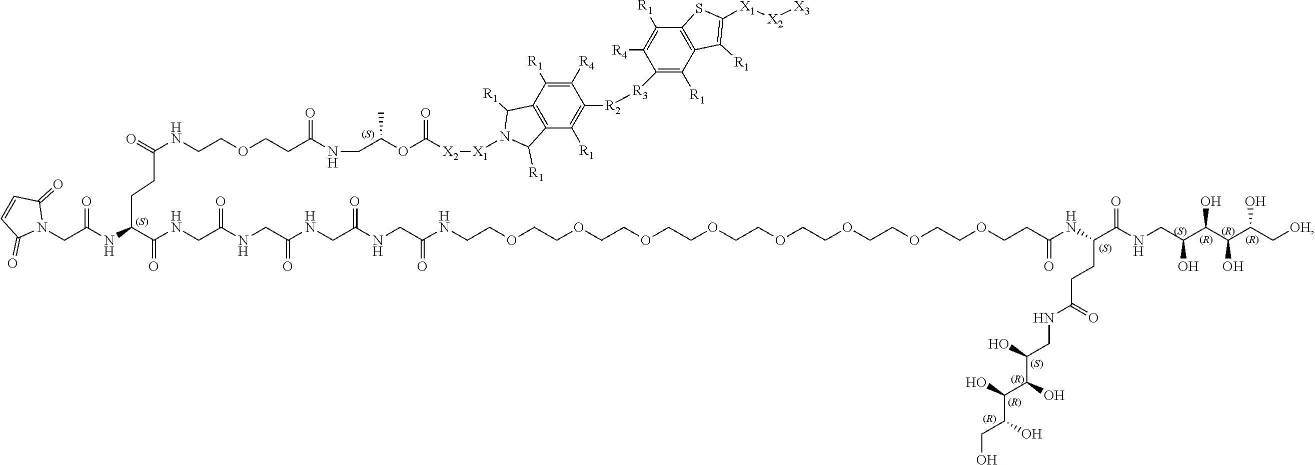

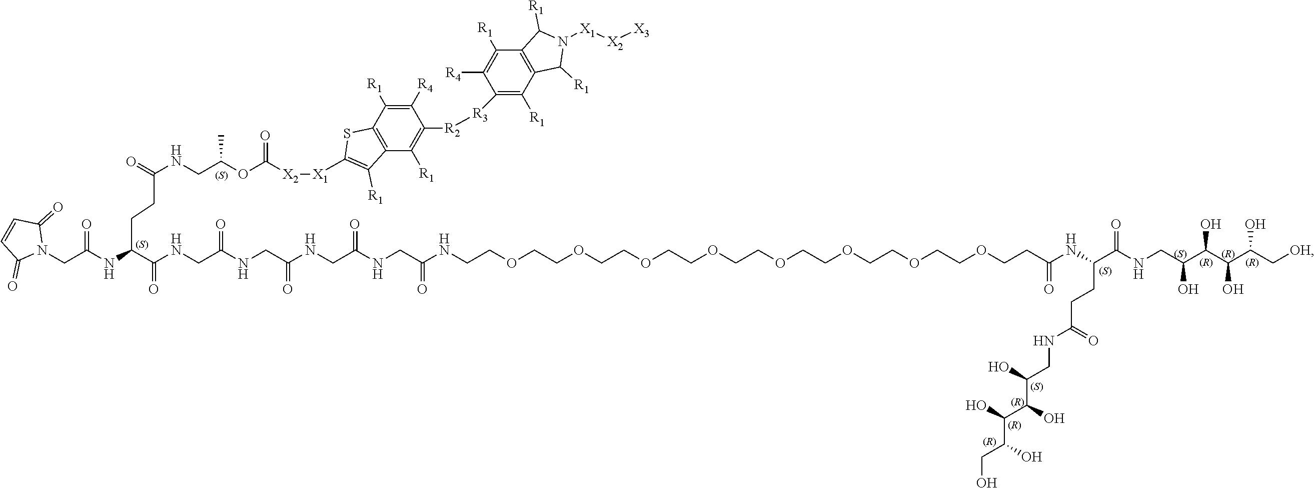

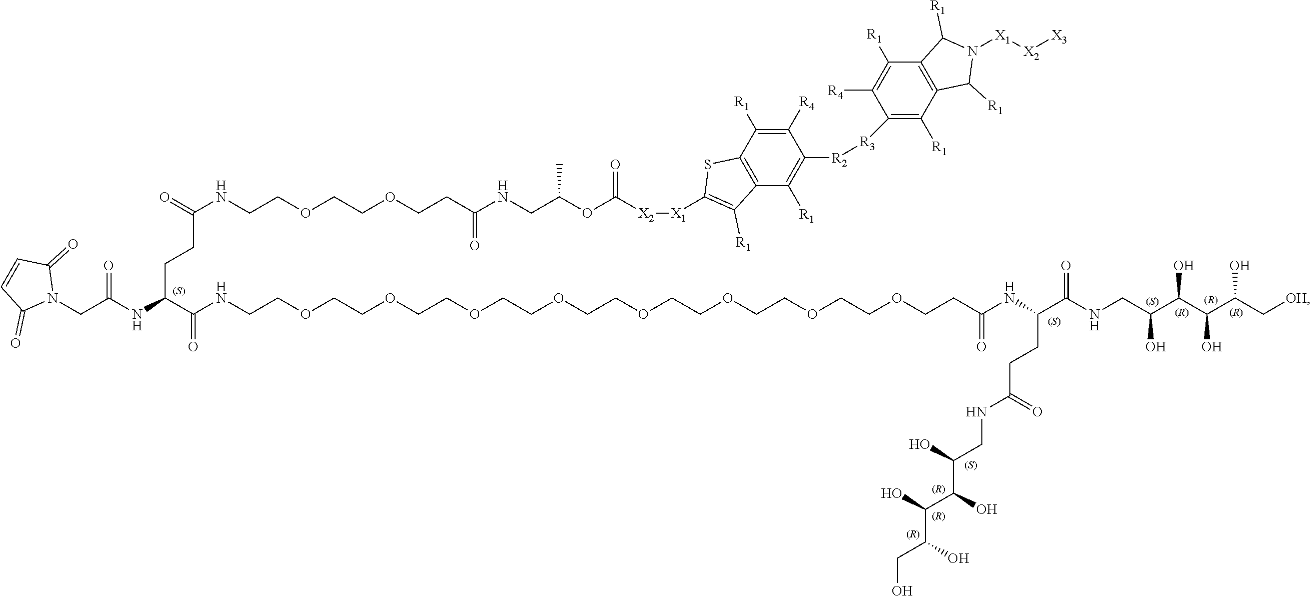

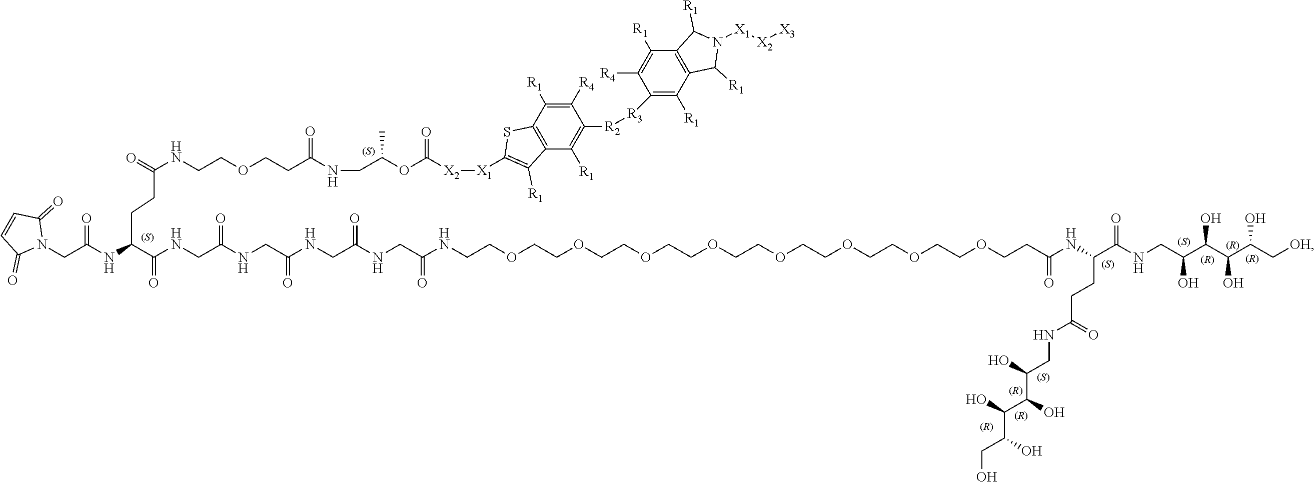

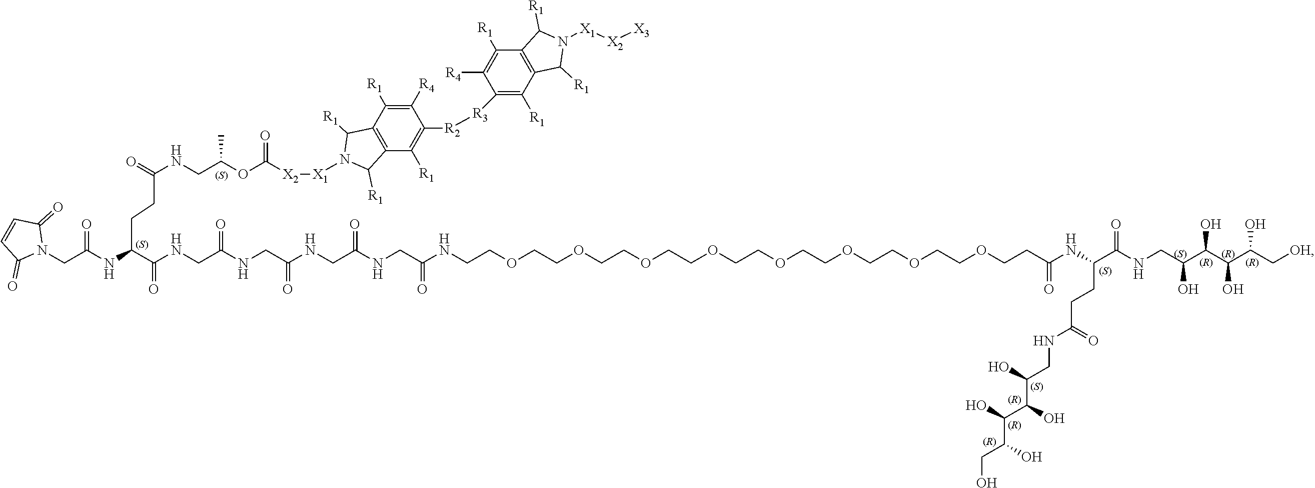

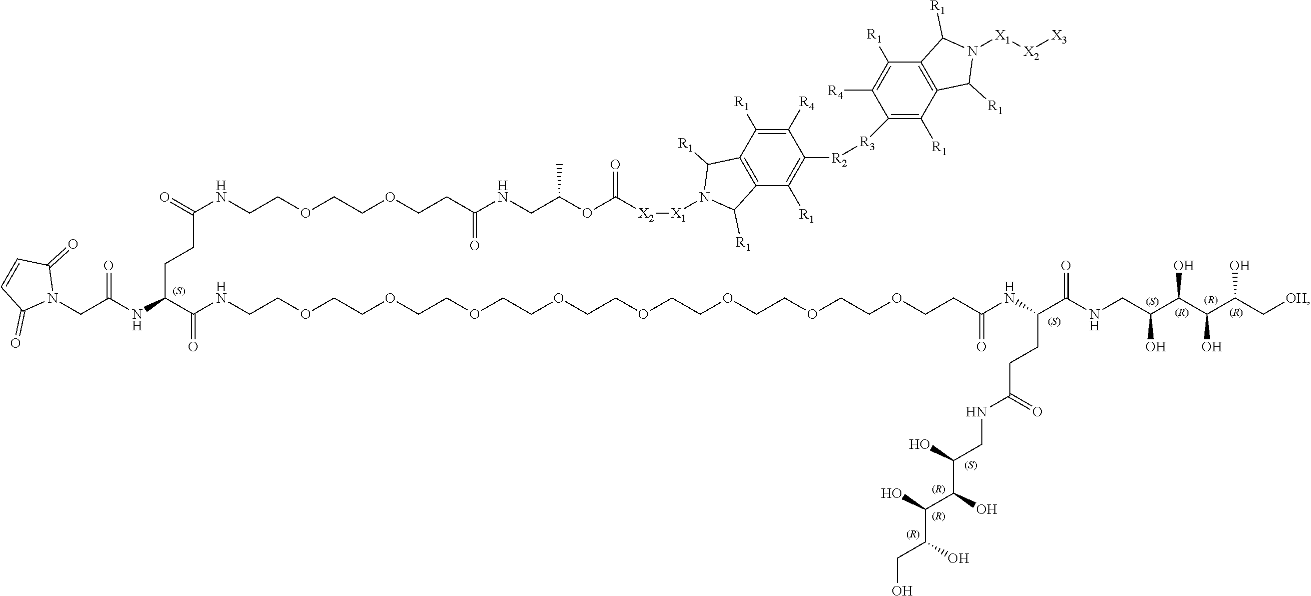

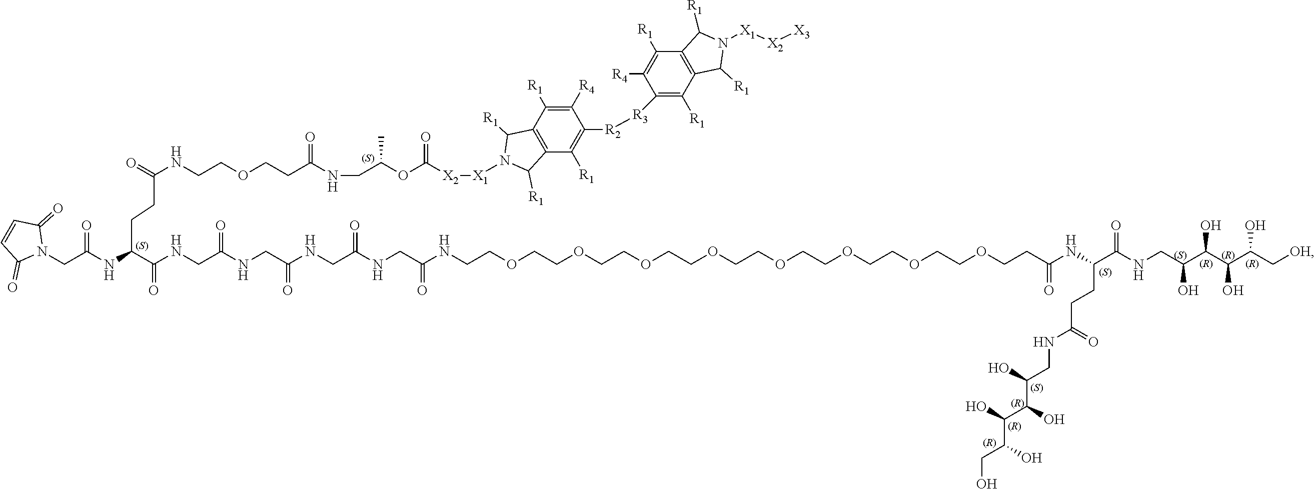

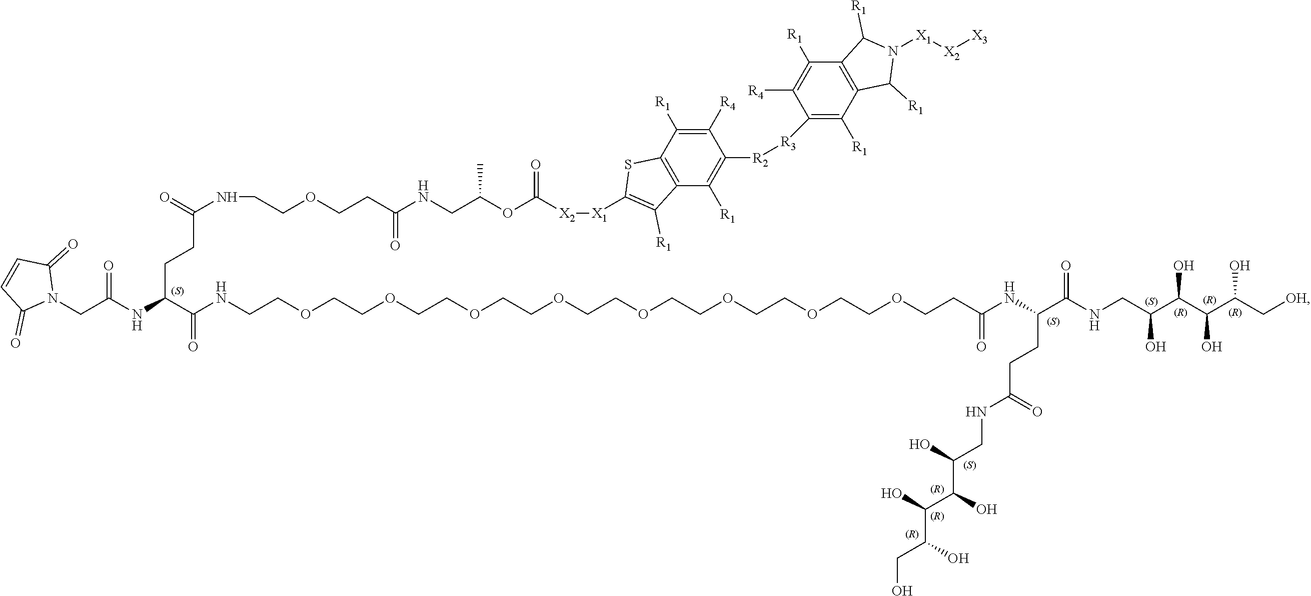

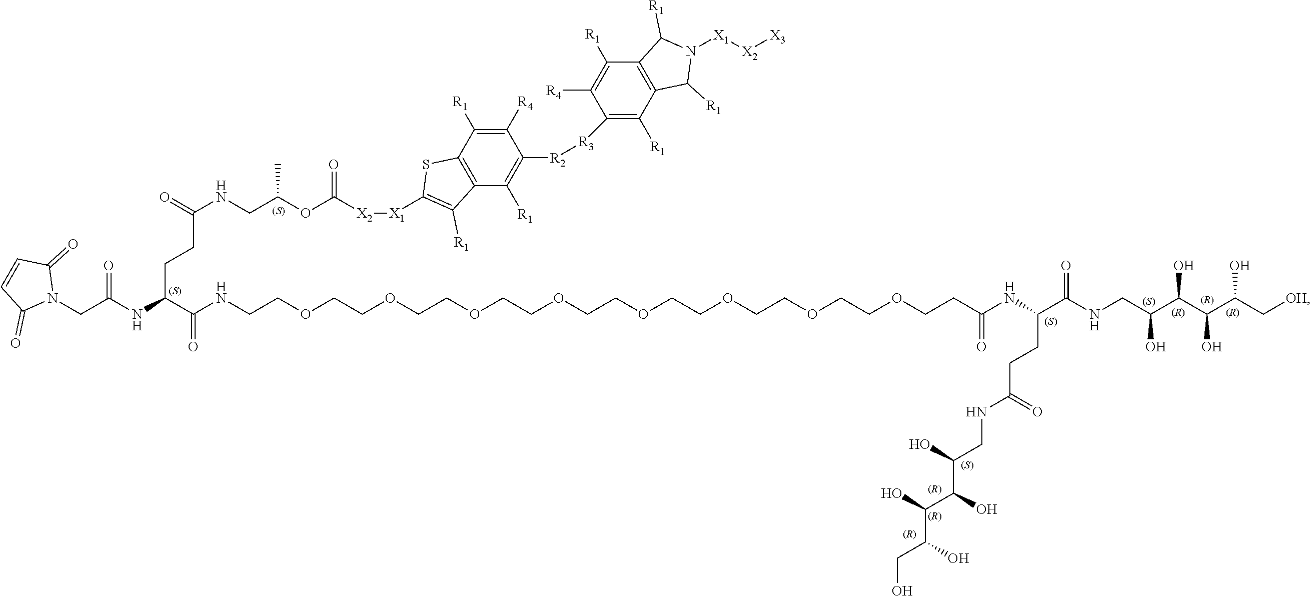

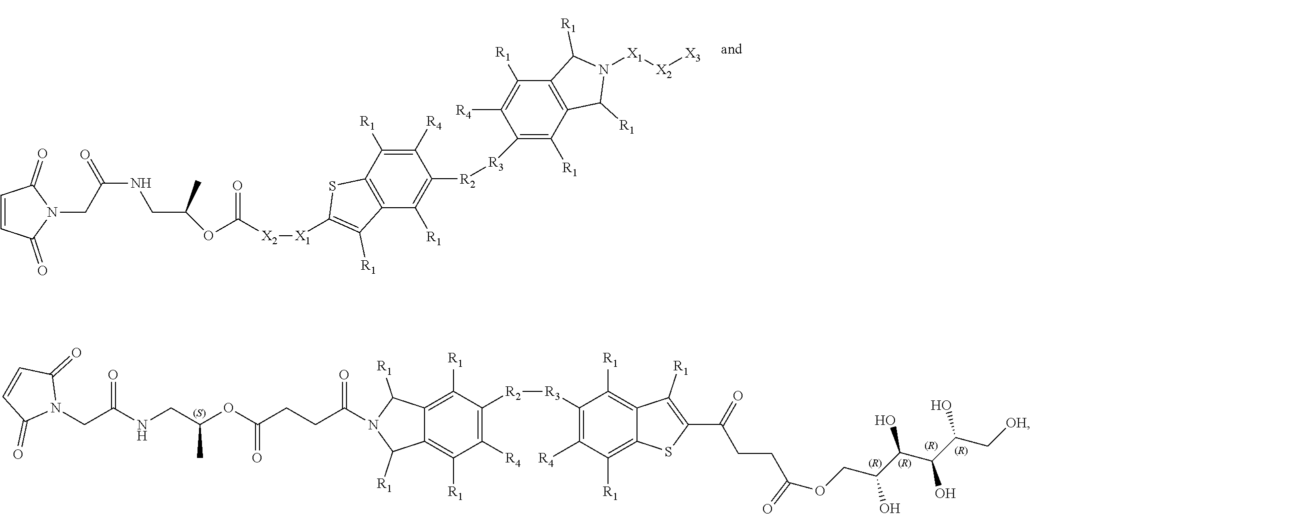

wherein each R₁, R₂, R₃, R₄, X₁, X₂ and X₃ is defined as above.

In some embodiments, the compound-linker construct of the present disclosure, is selected from the group consisting of:

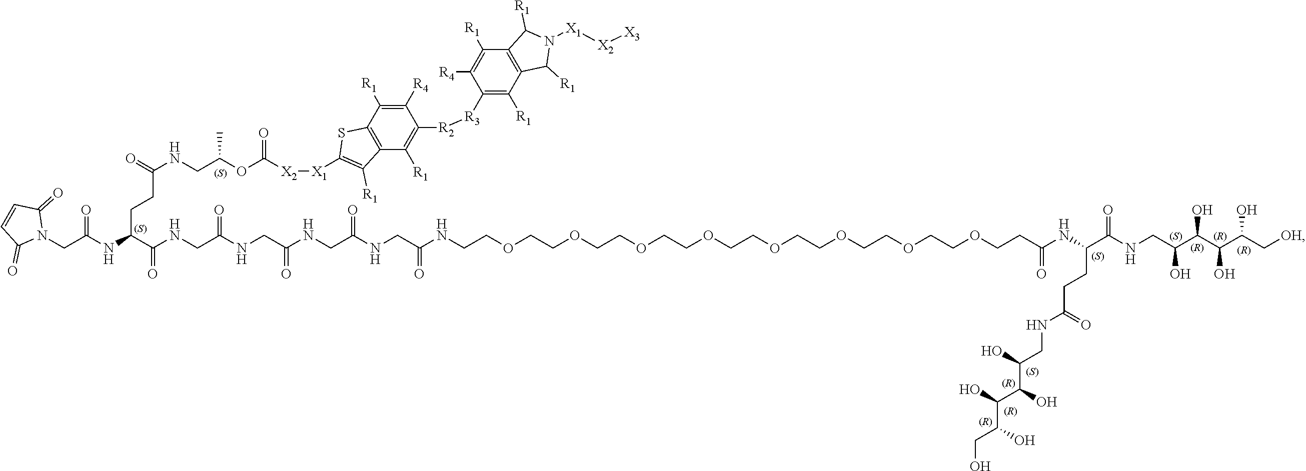

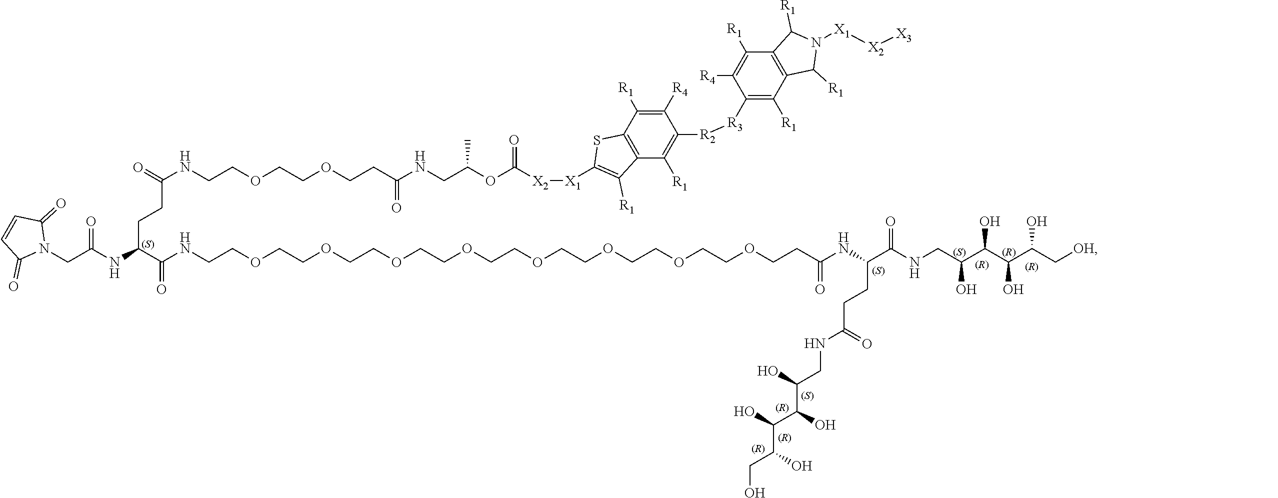

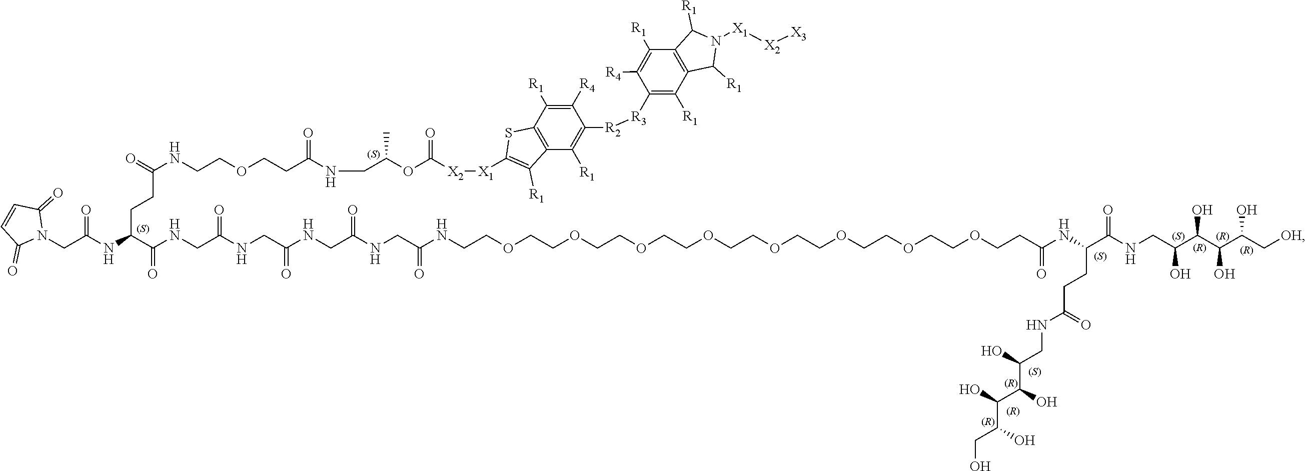

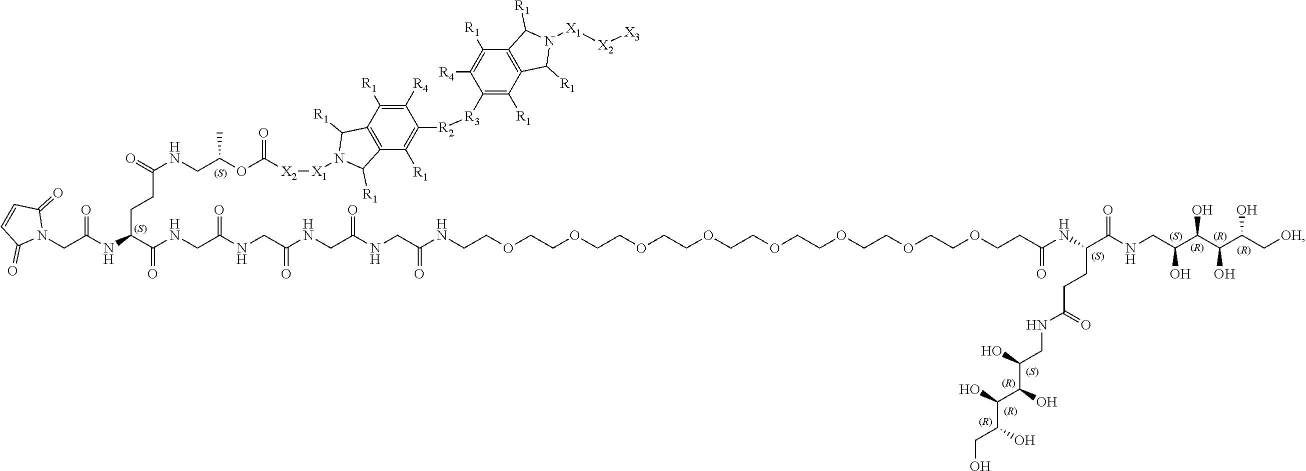

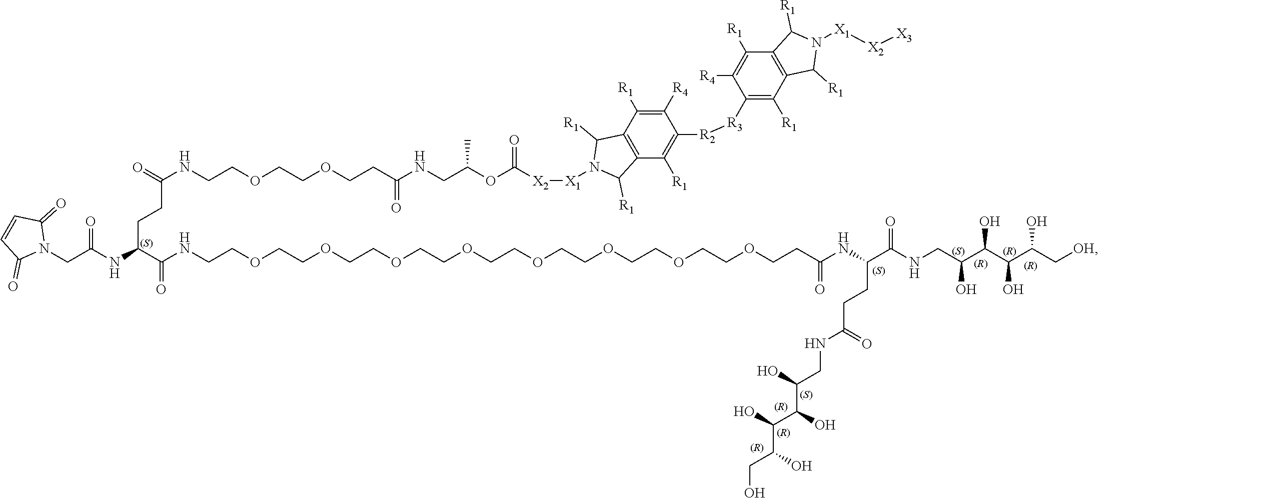

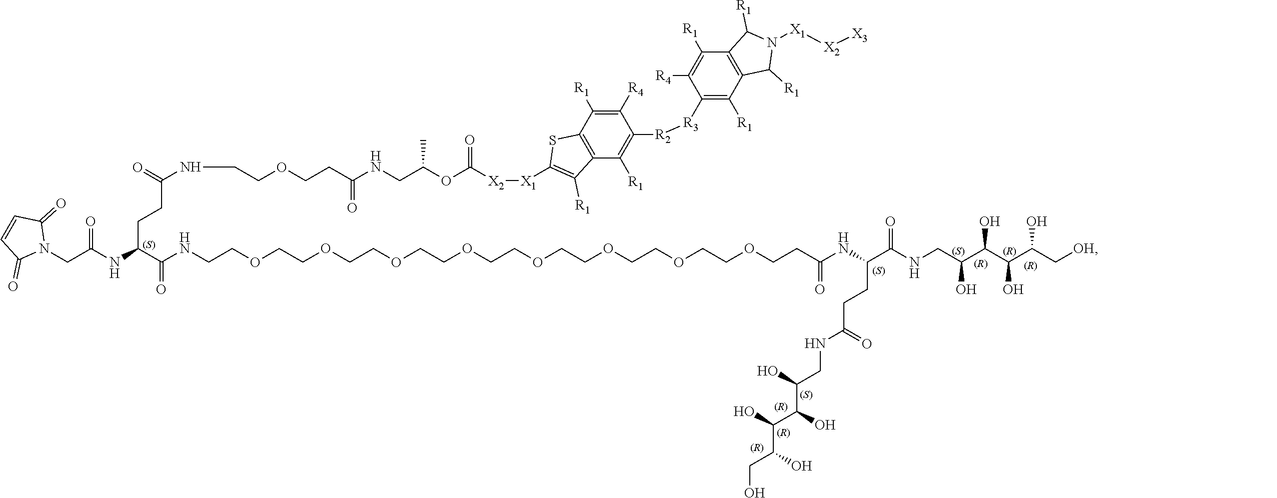

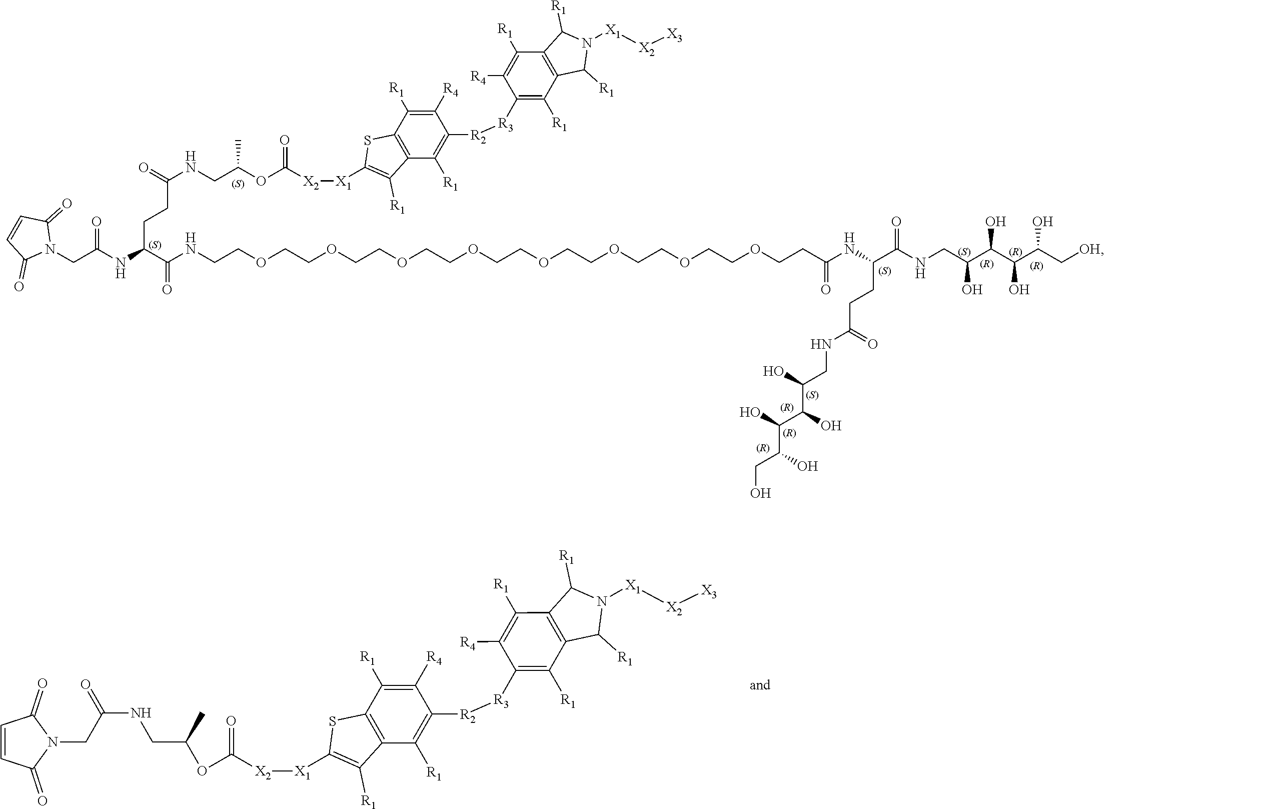

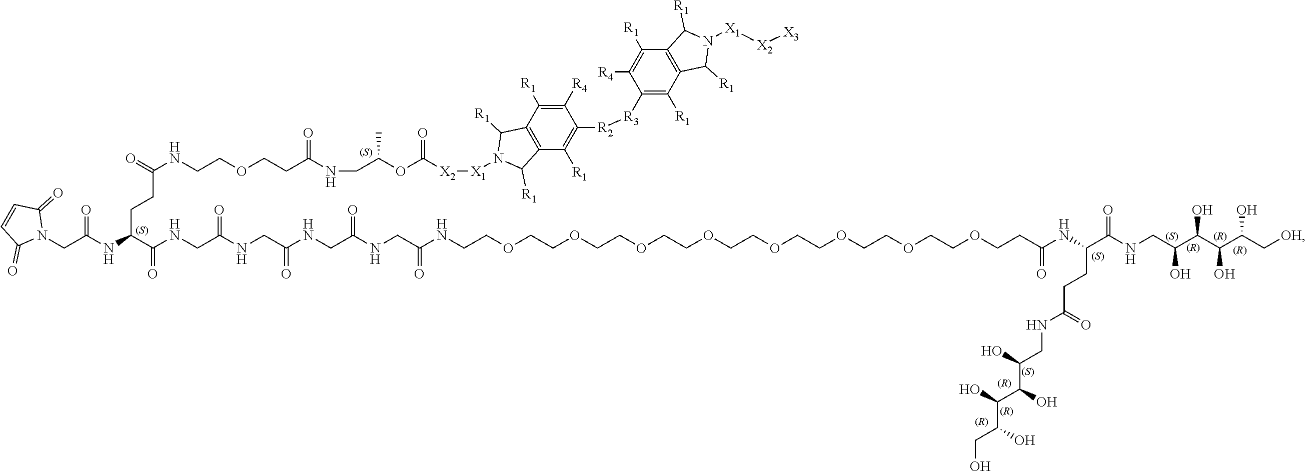

wherein each R₁, R₂, R₃, R₄, X₁, X₂ and X₃ is defined as above.

In some embodiments, the compound-linker construct of the present disclosure, is selected from the group consisting of:

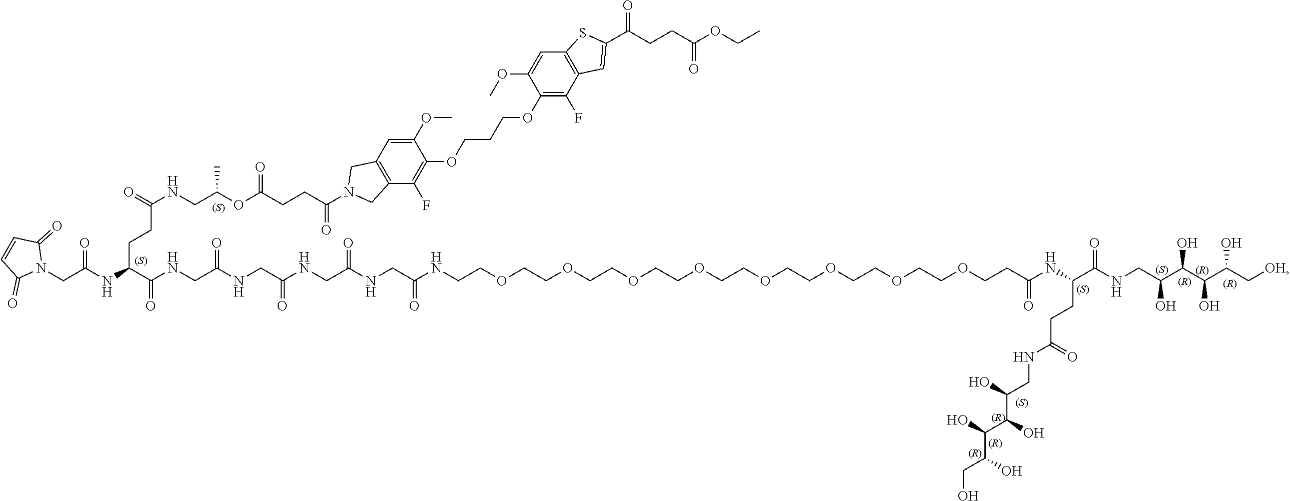

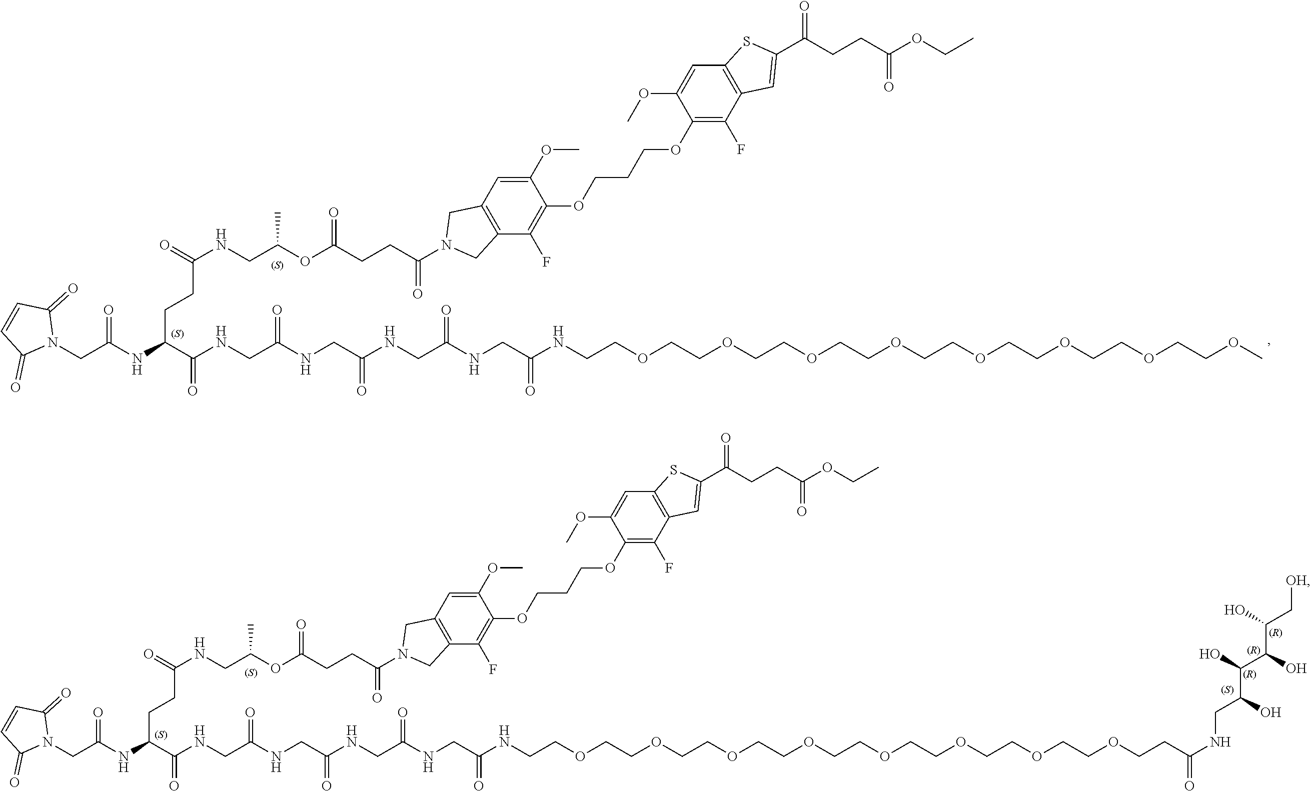

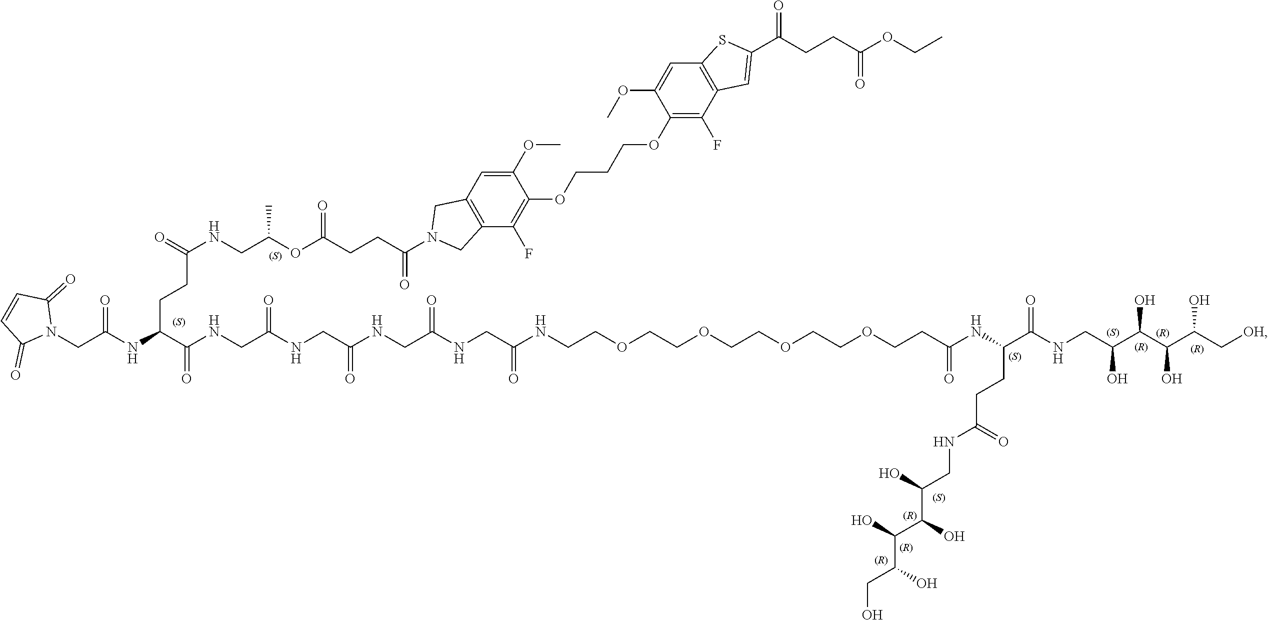

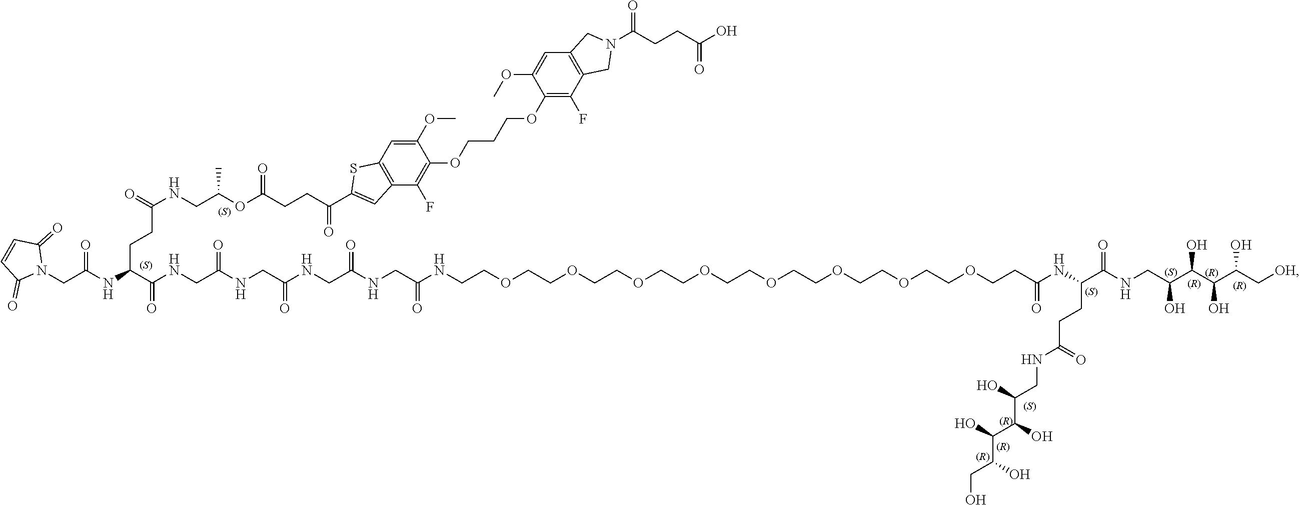

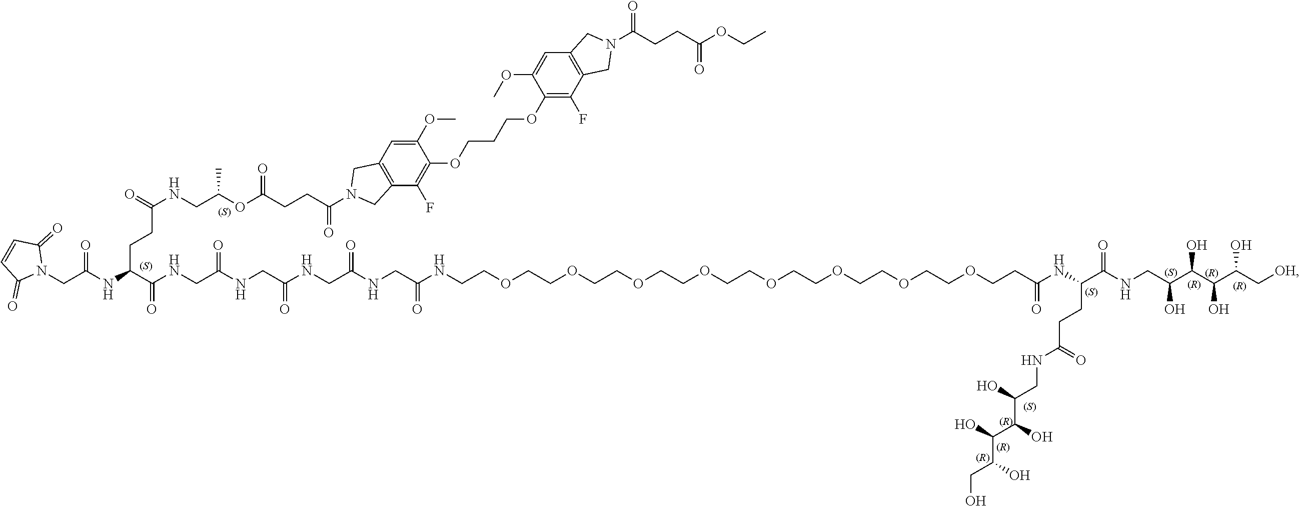

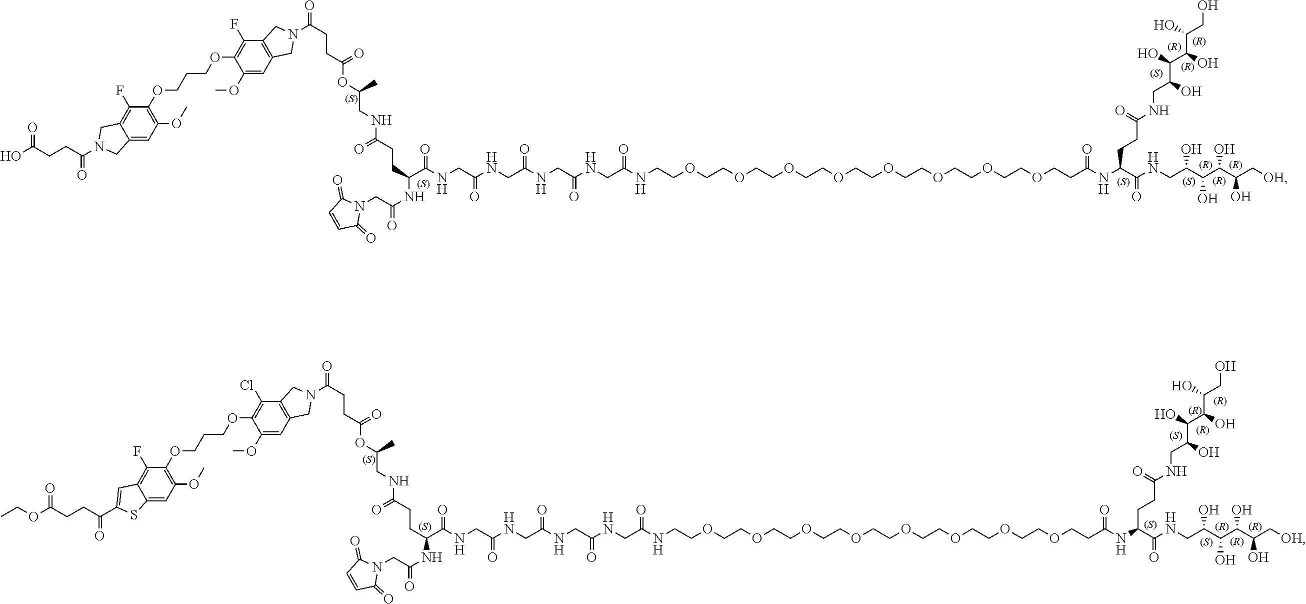

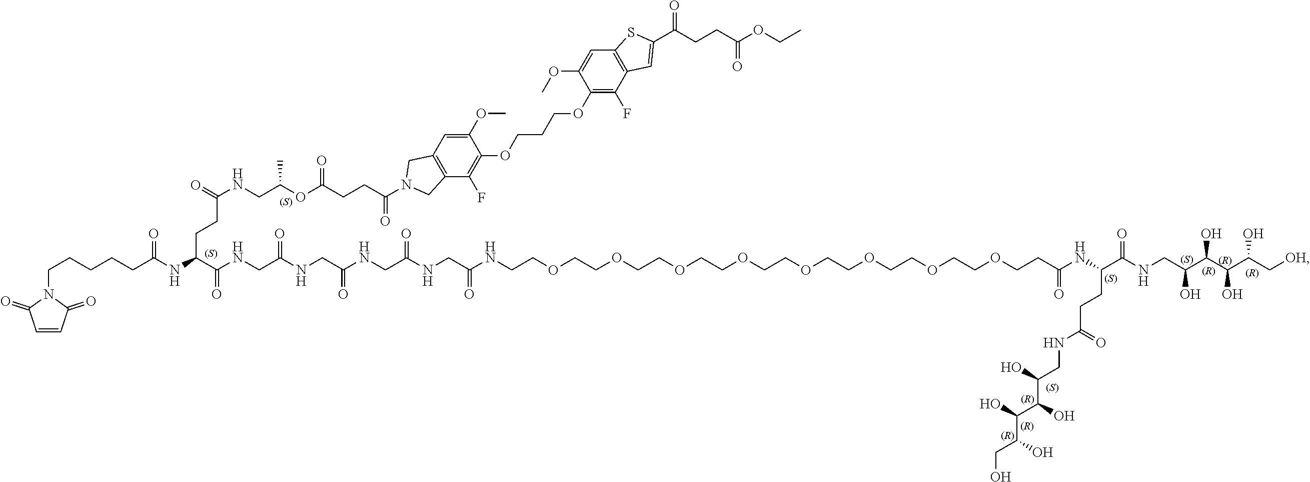

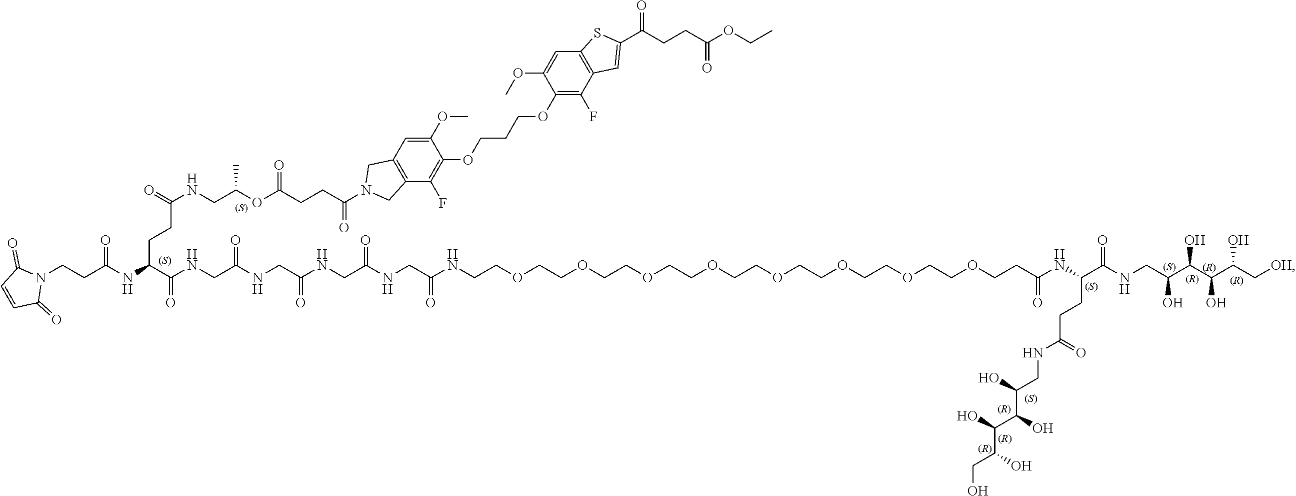

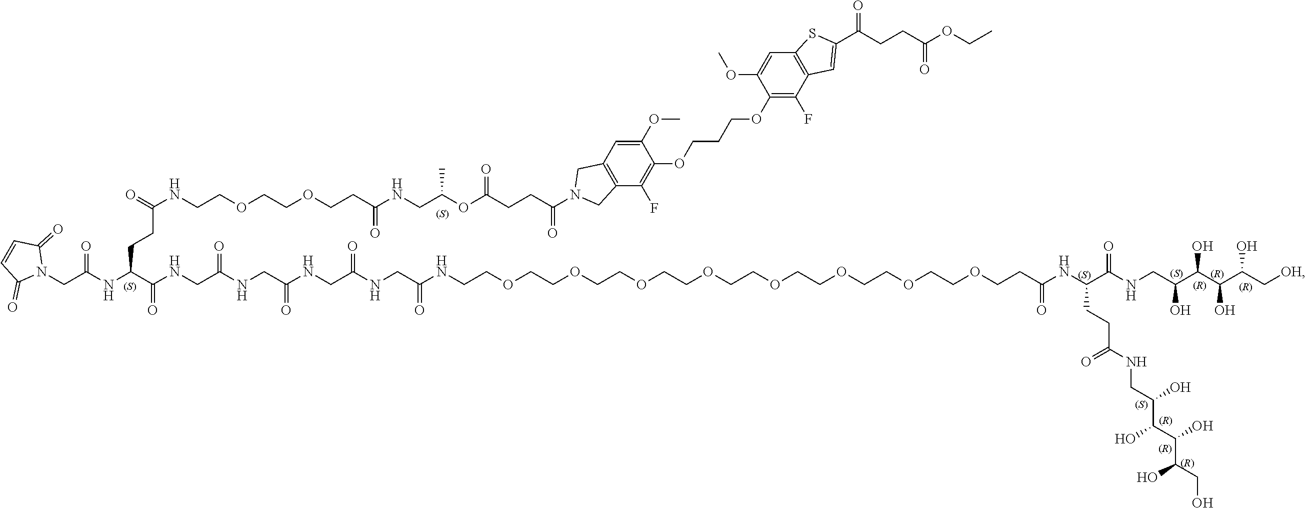

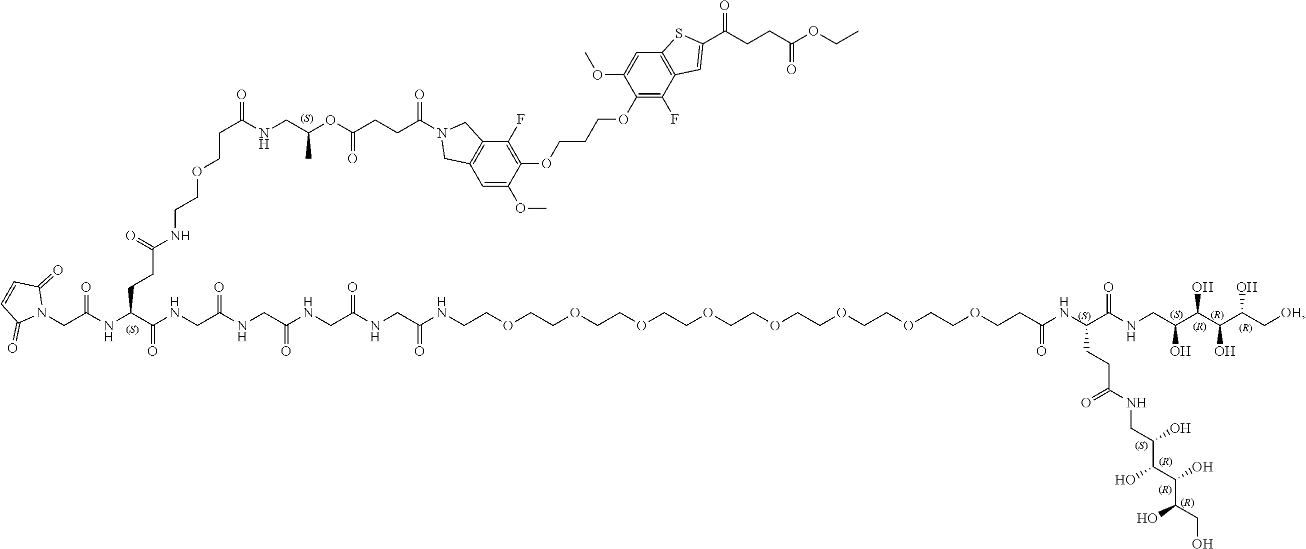

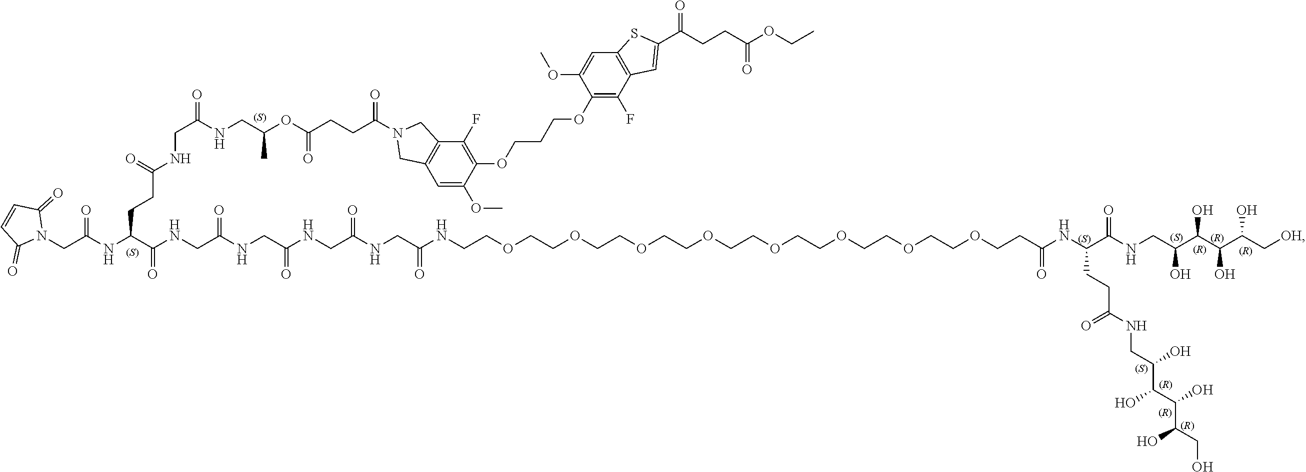

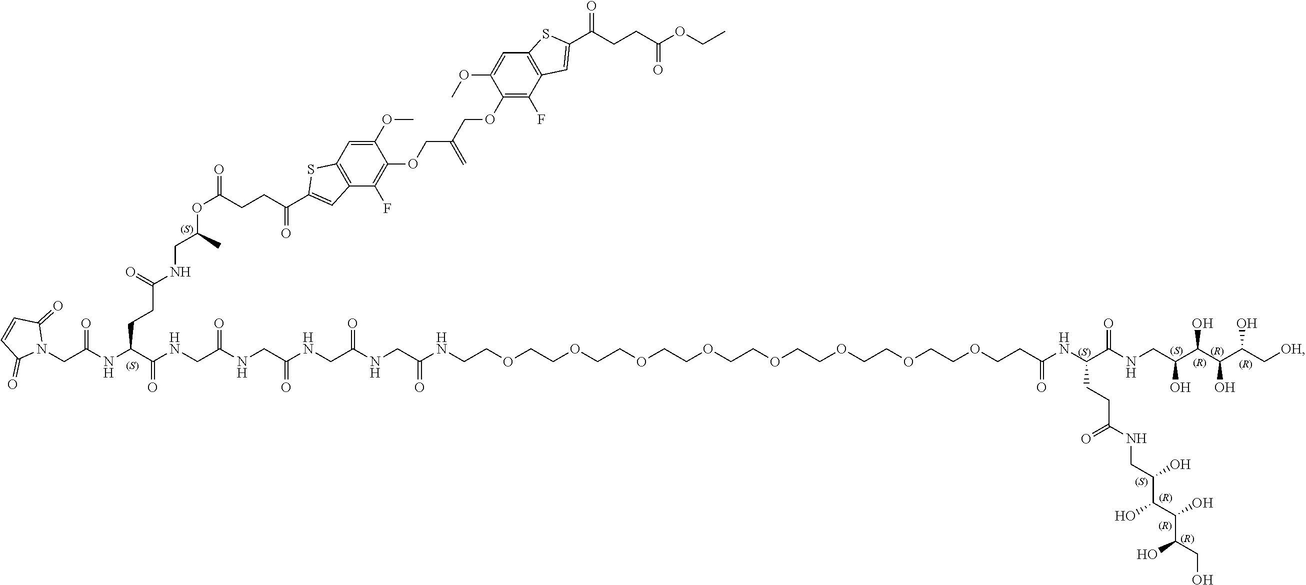

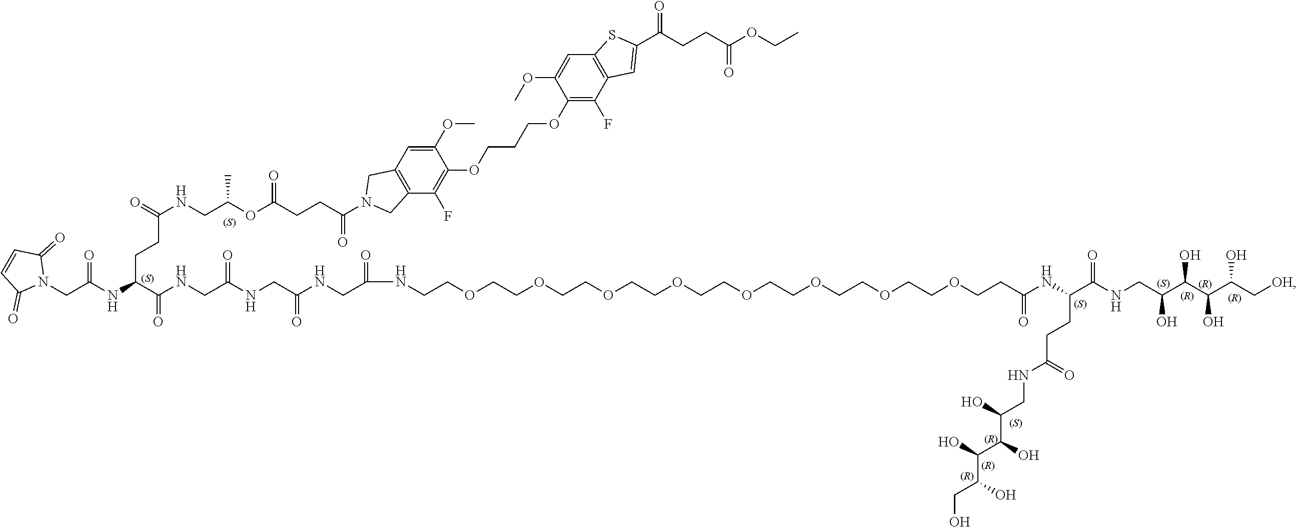

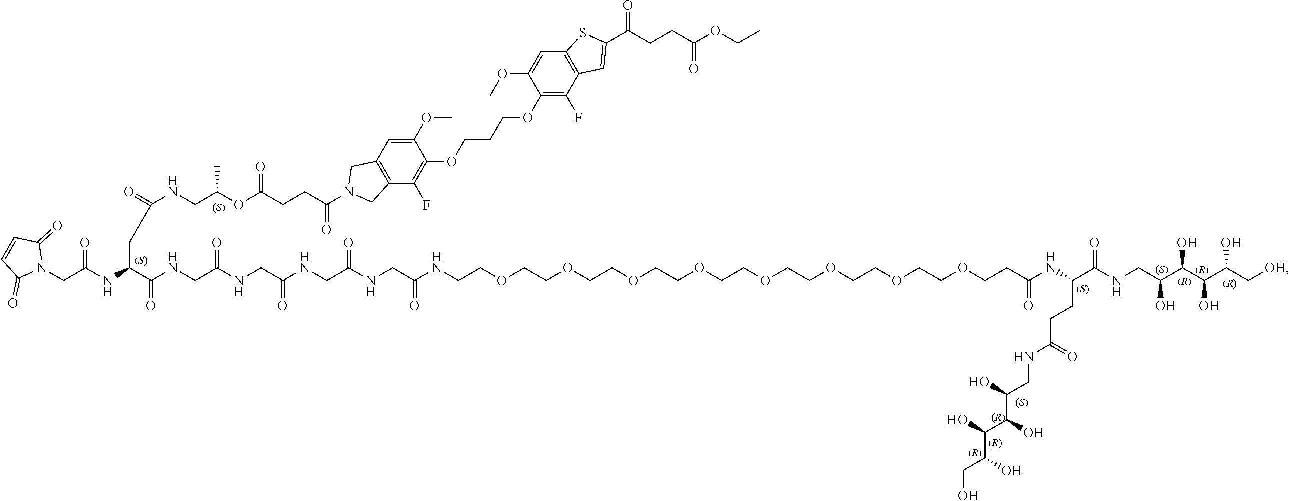

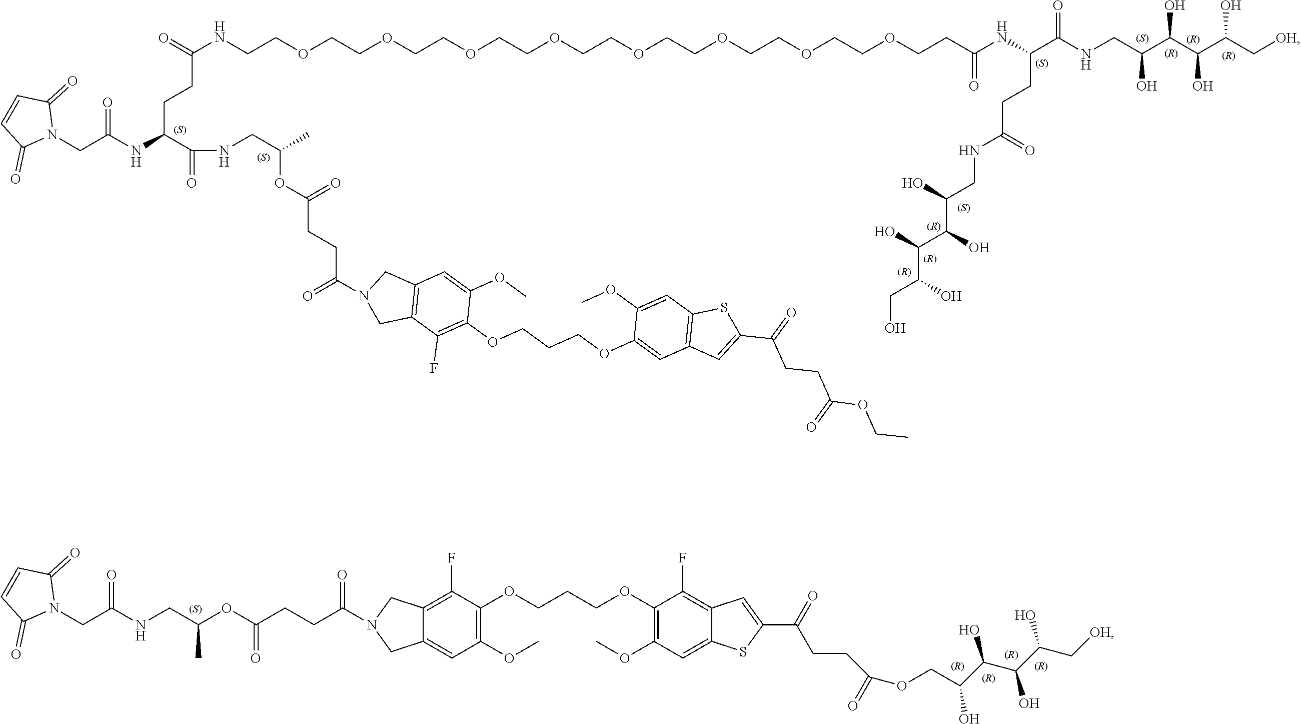

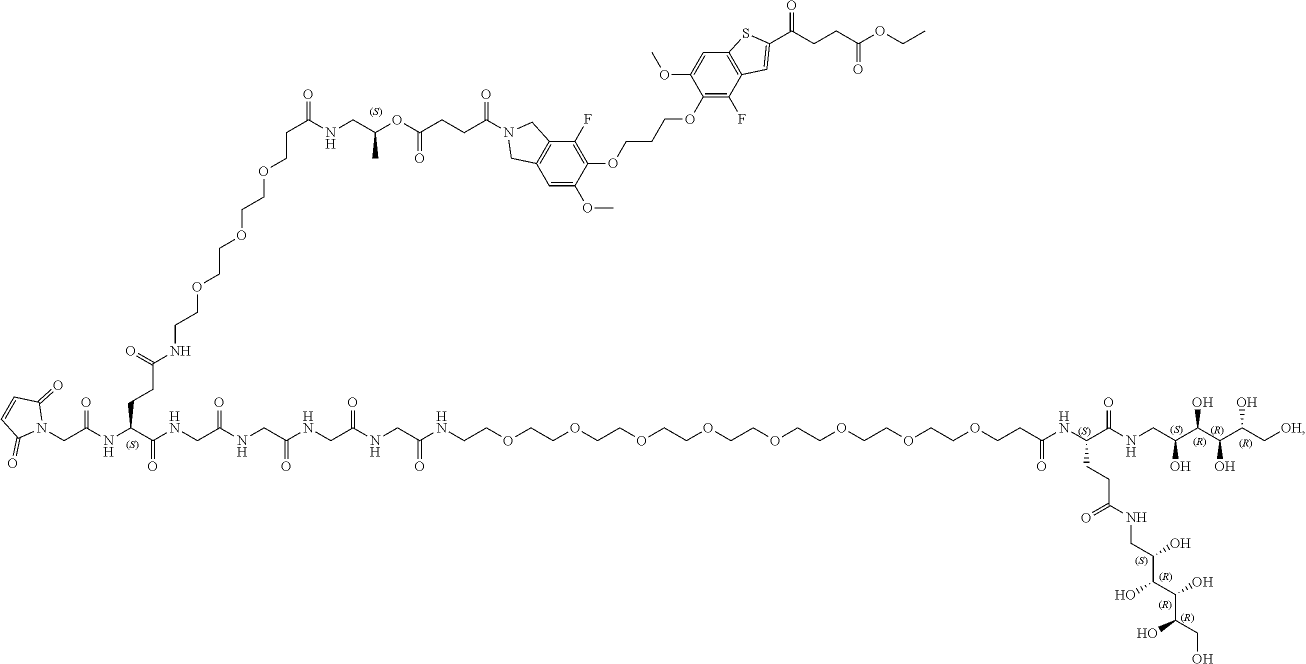

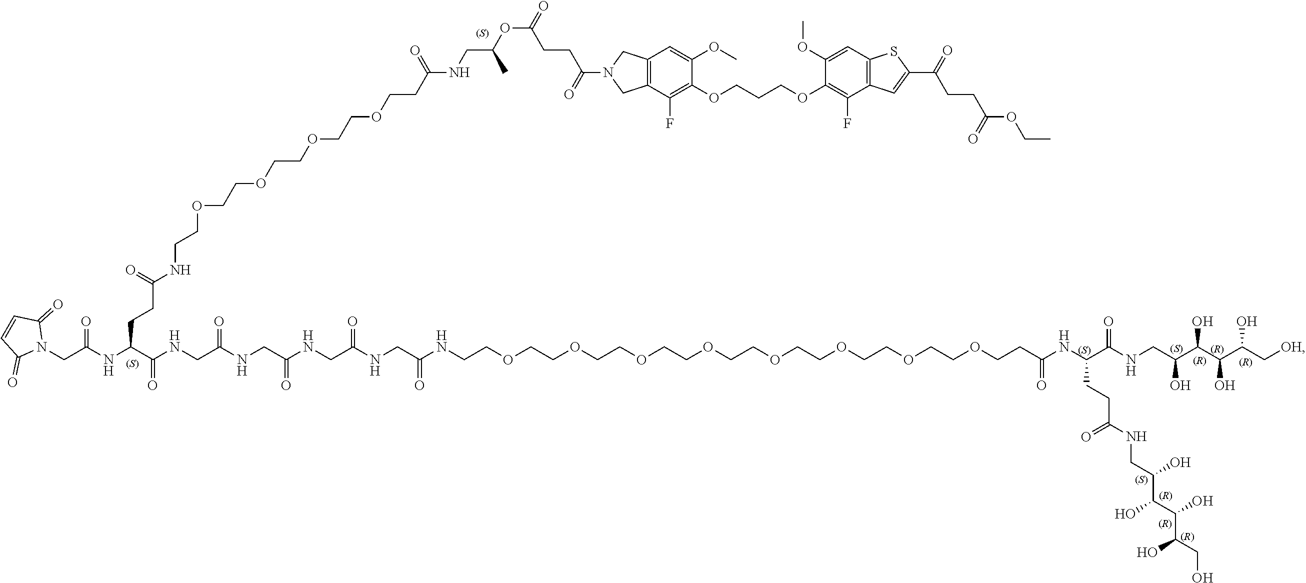

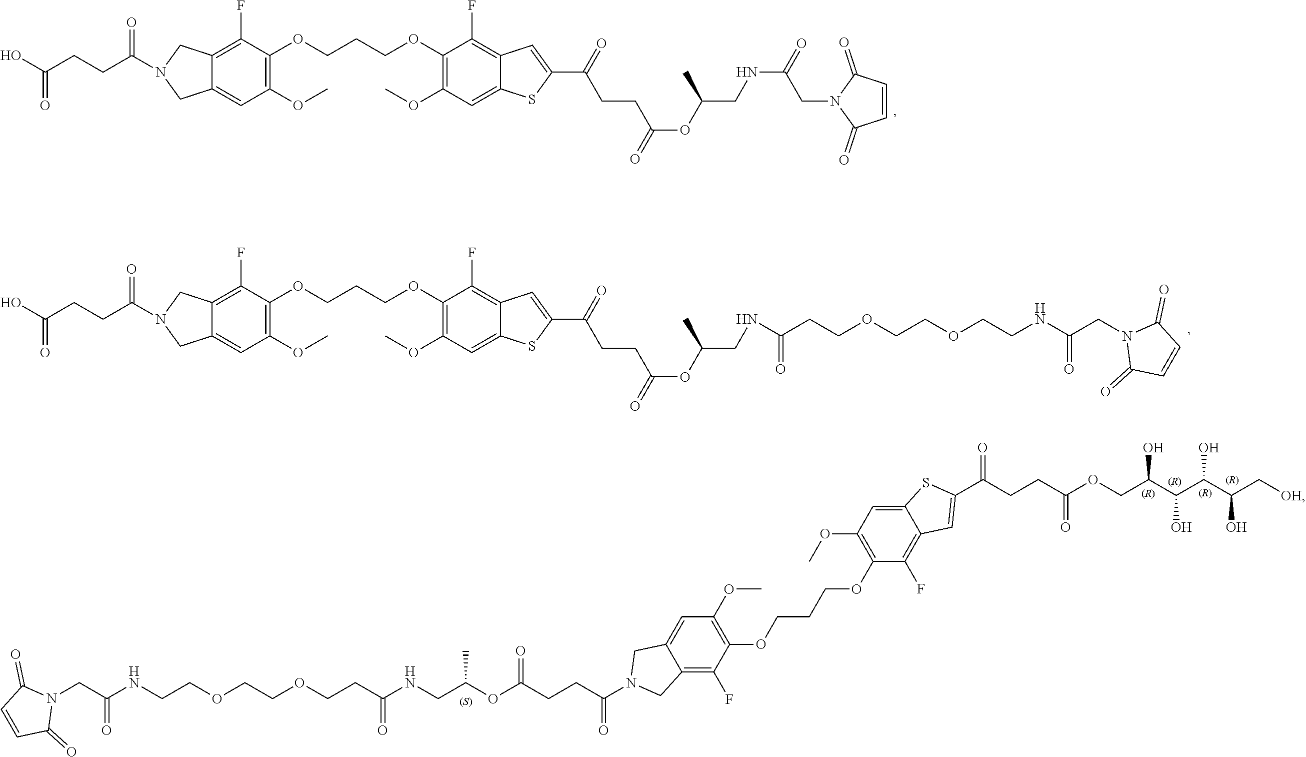

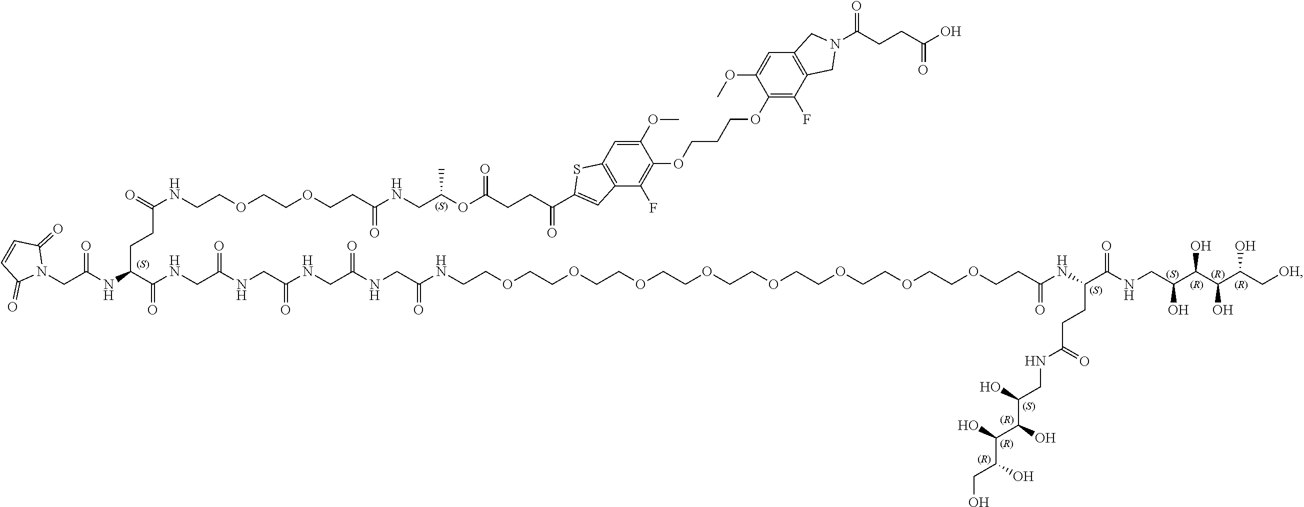

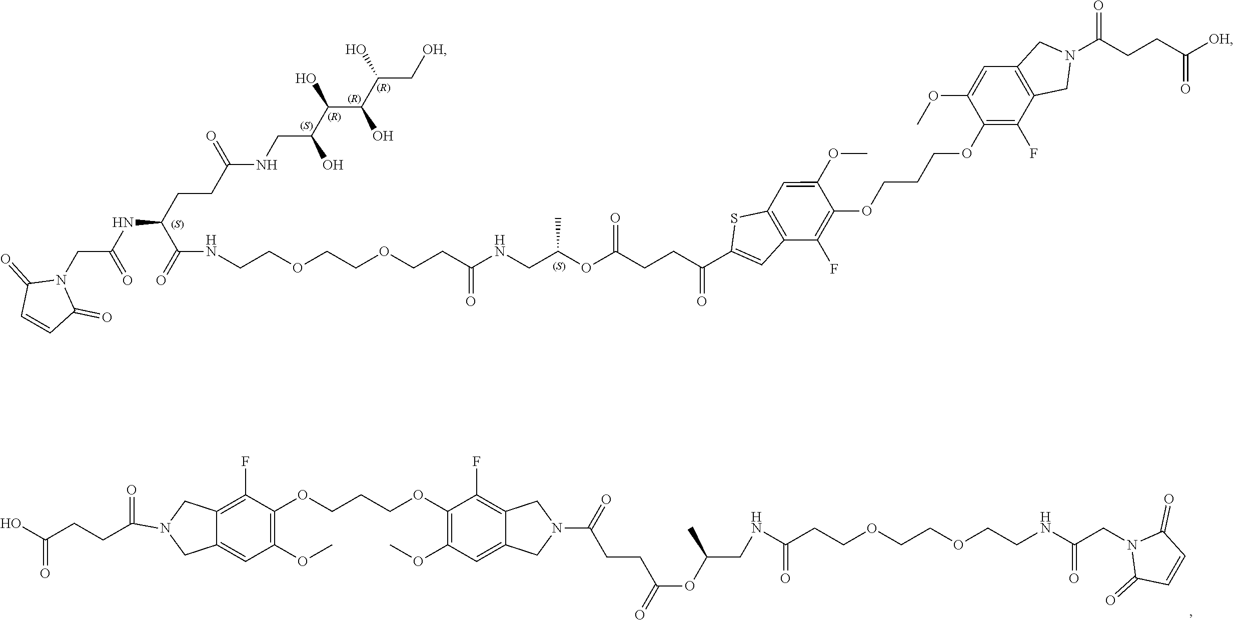

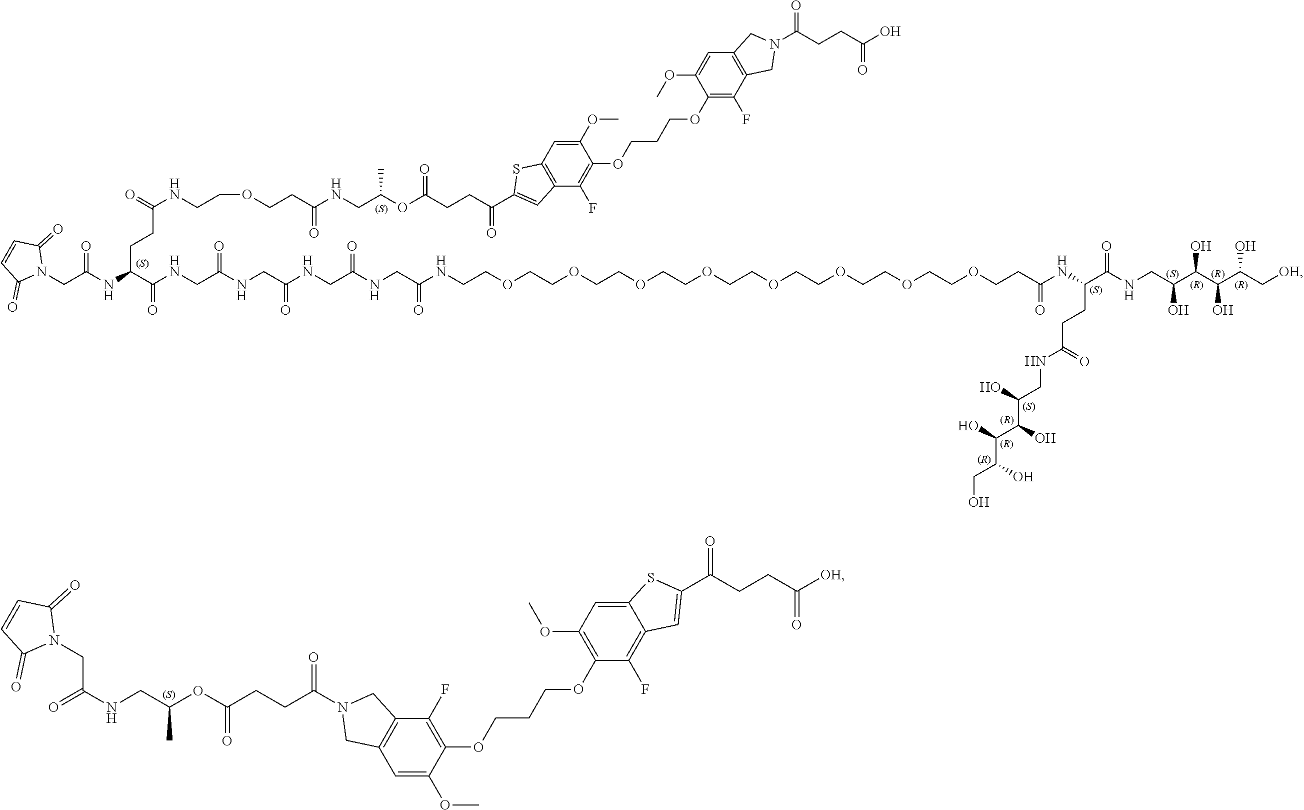

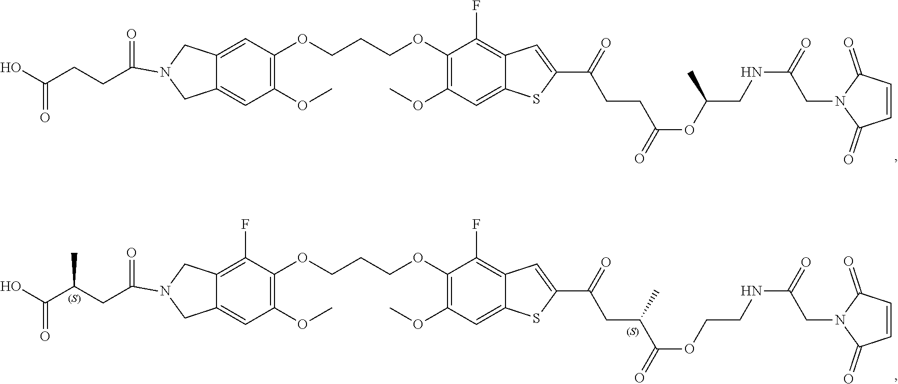

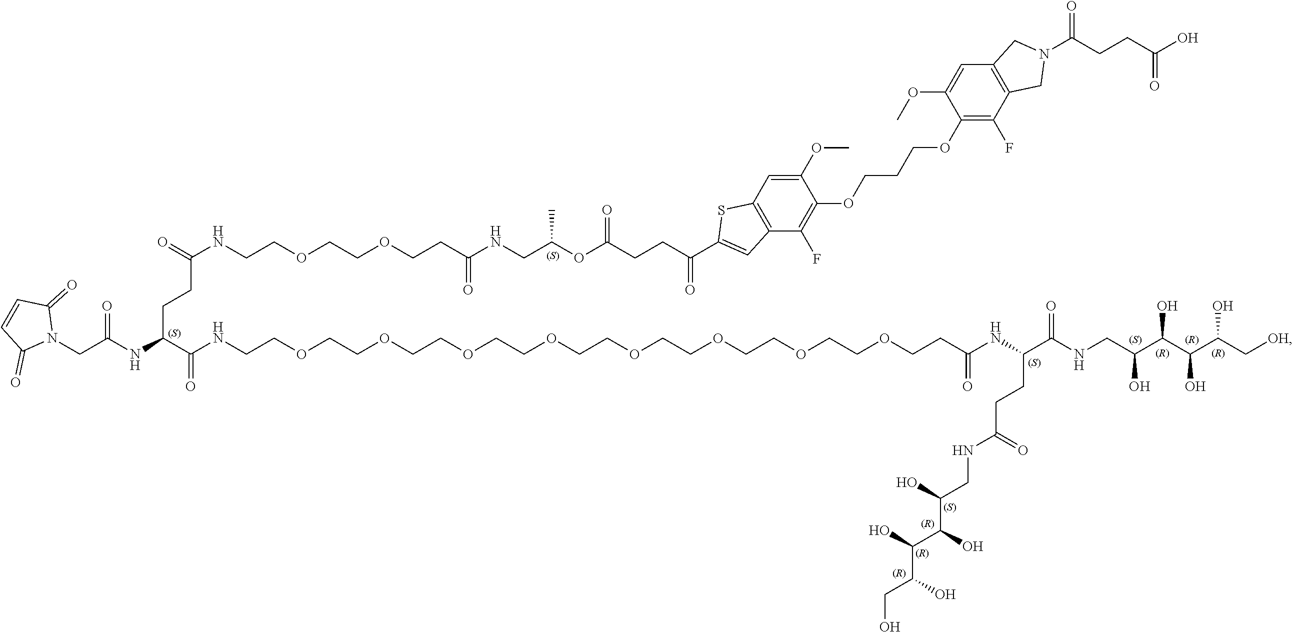

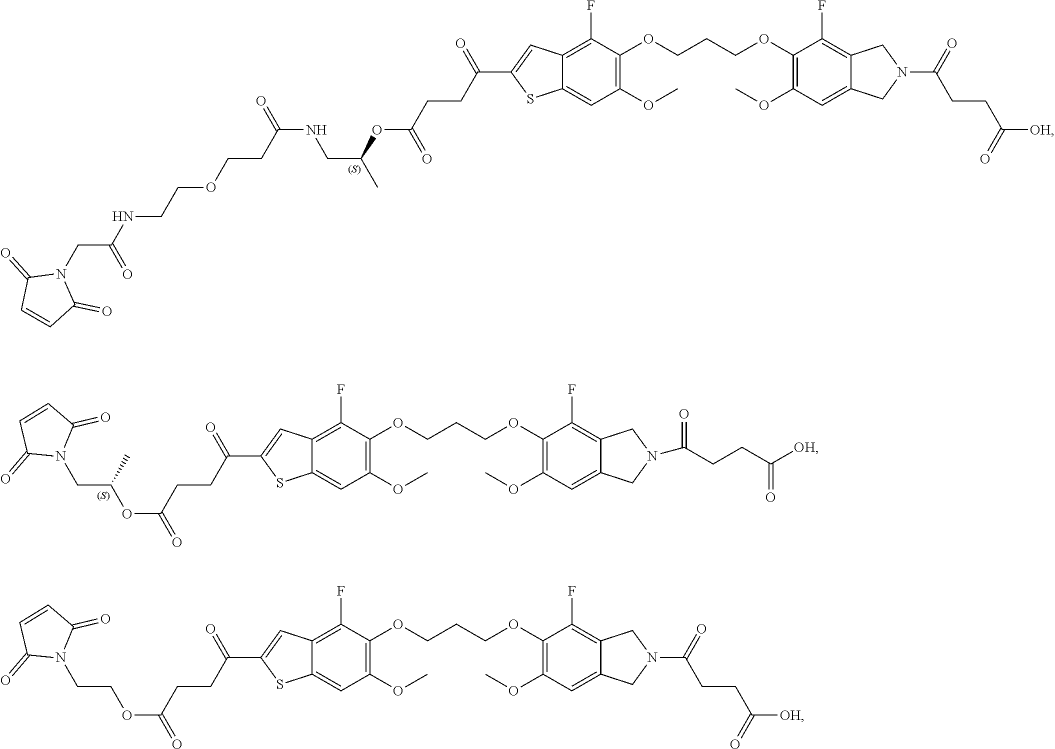

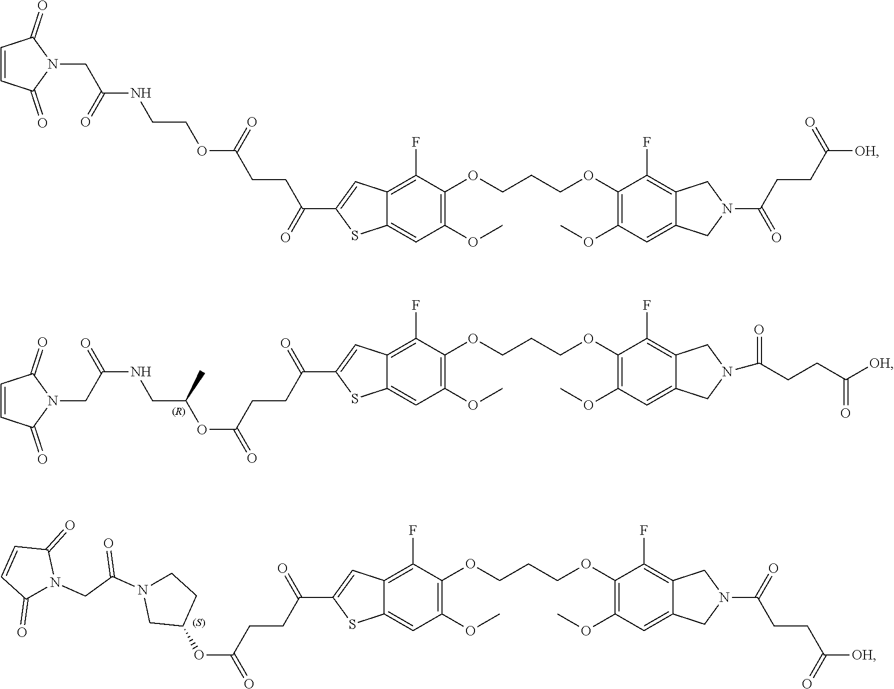

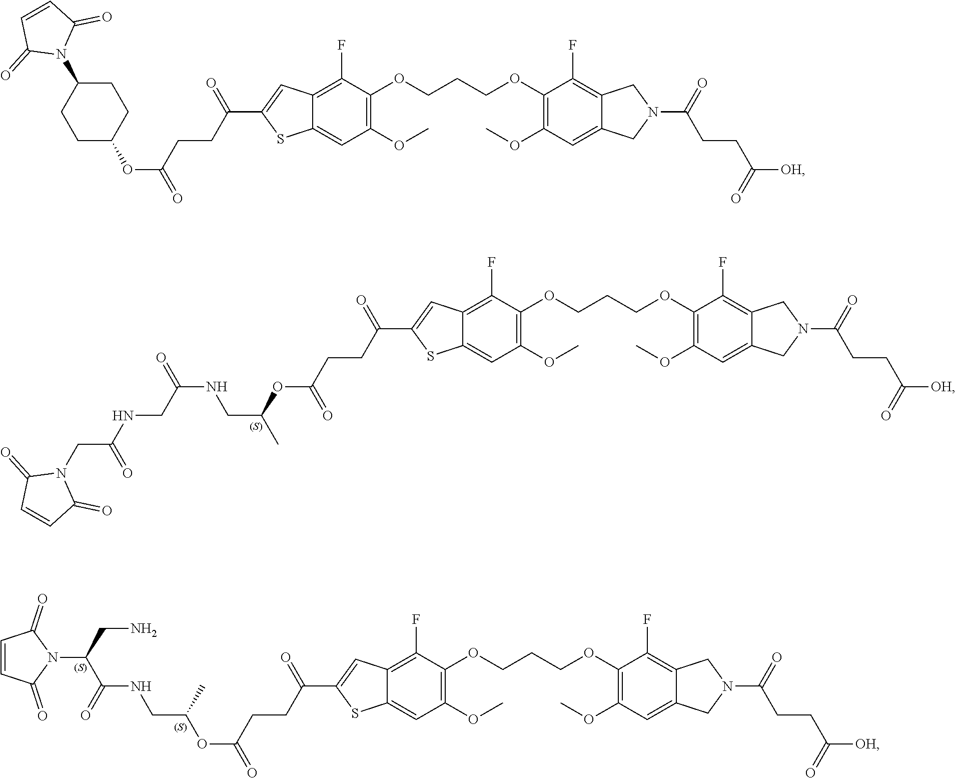

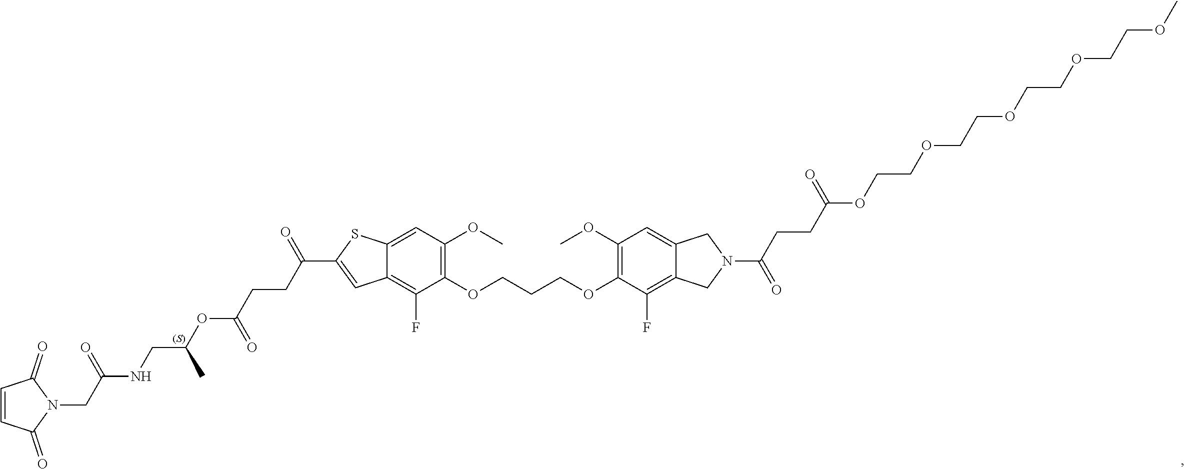

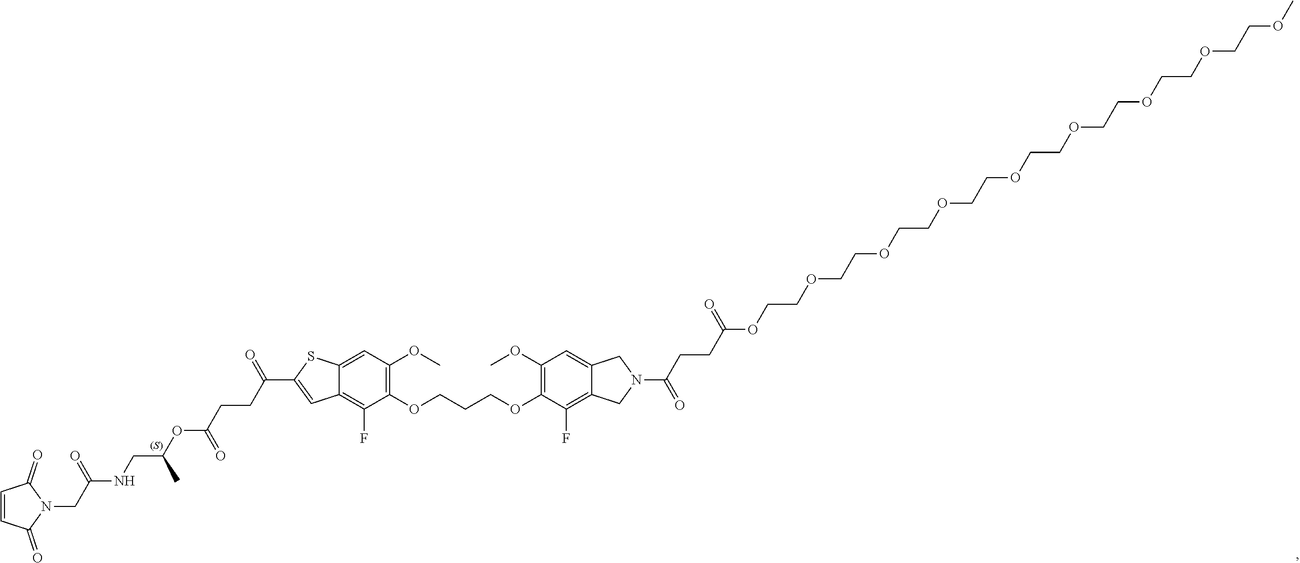

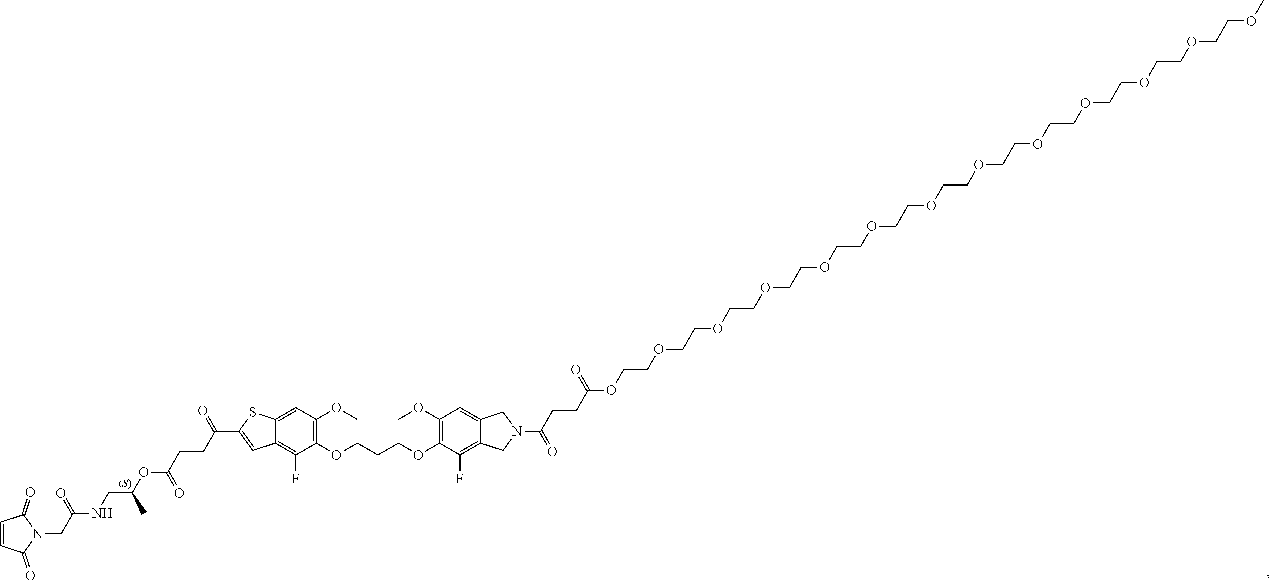

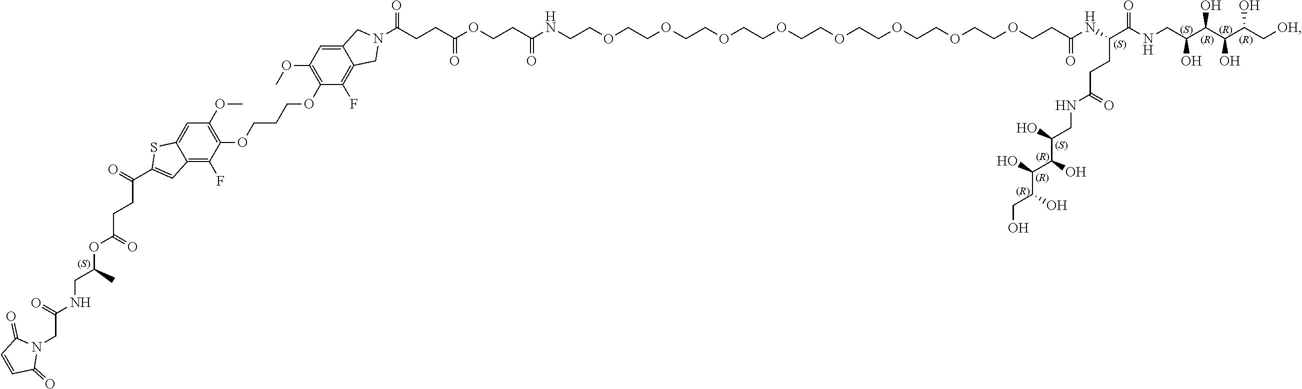

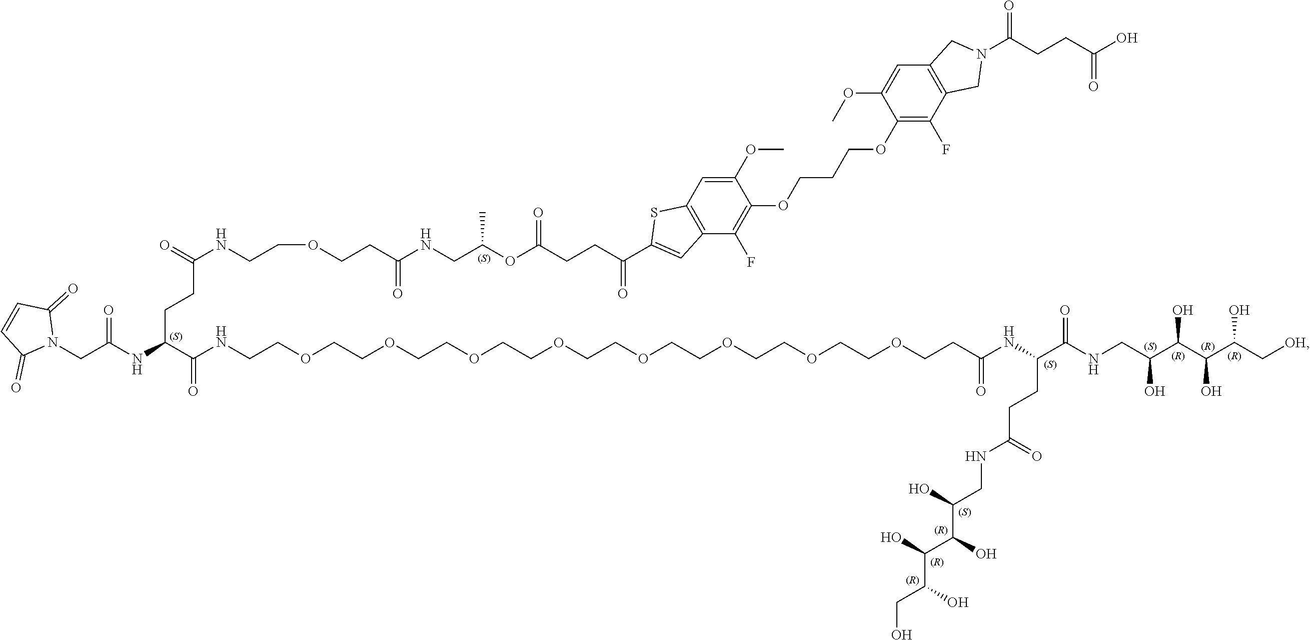

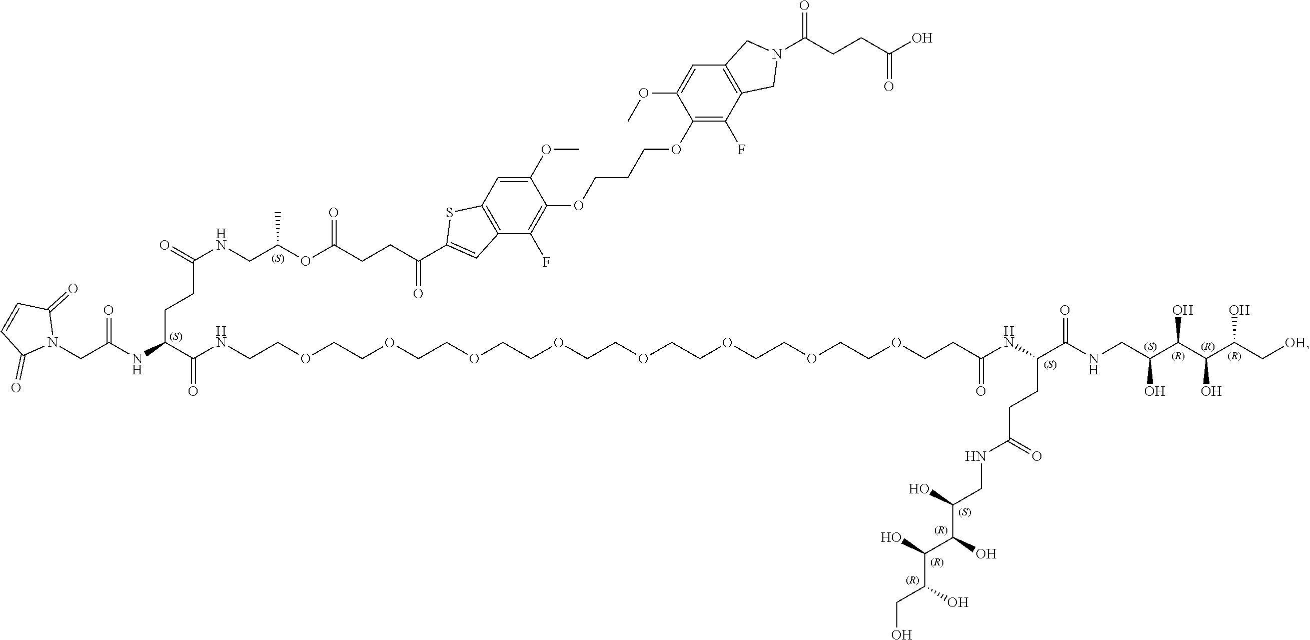

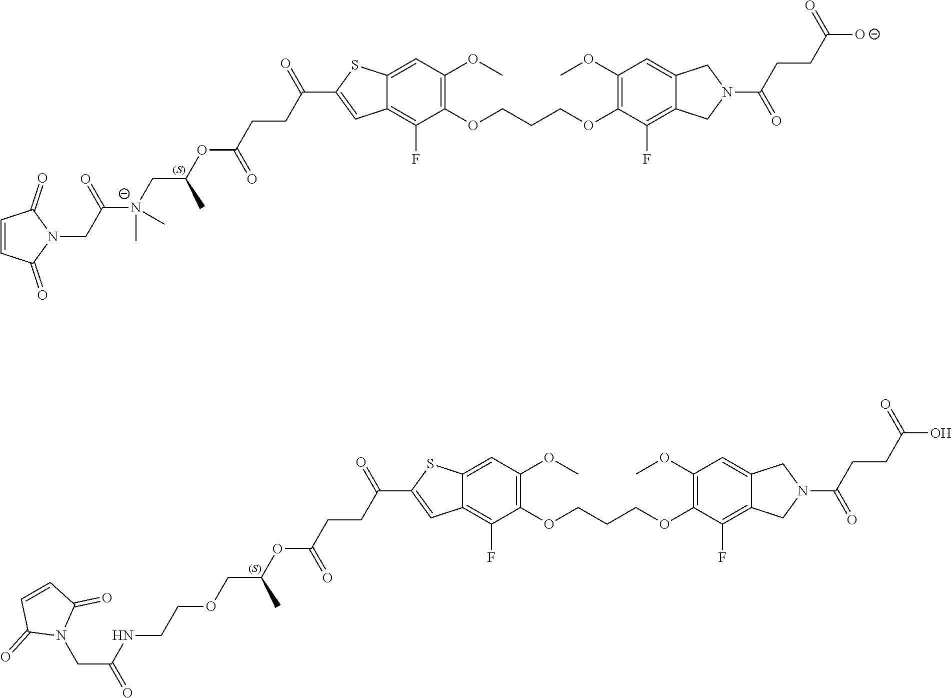

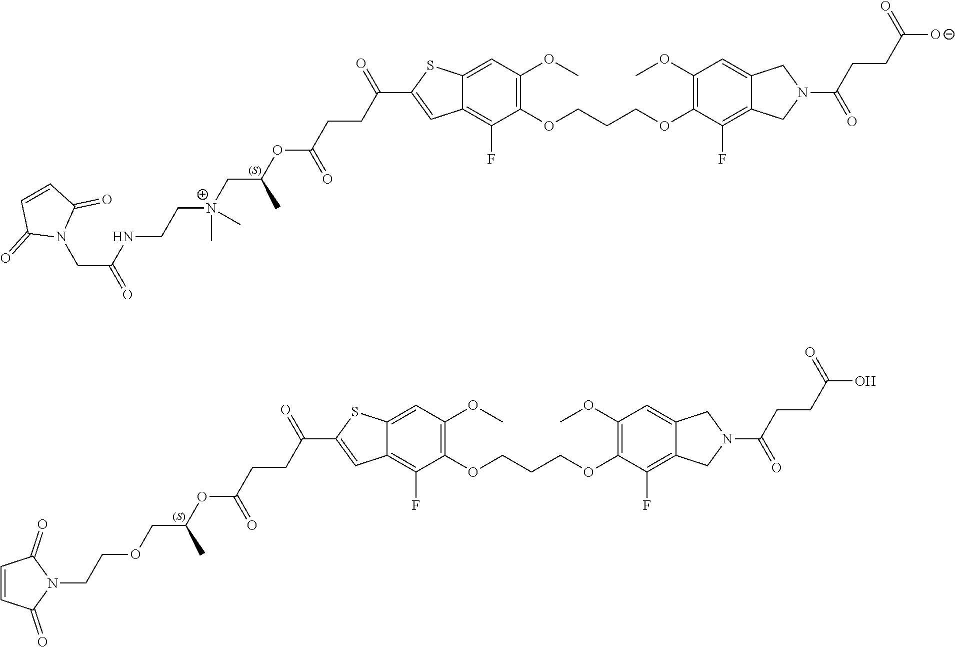

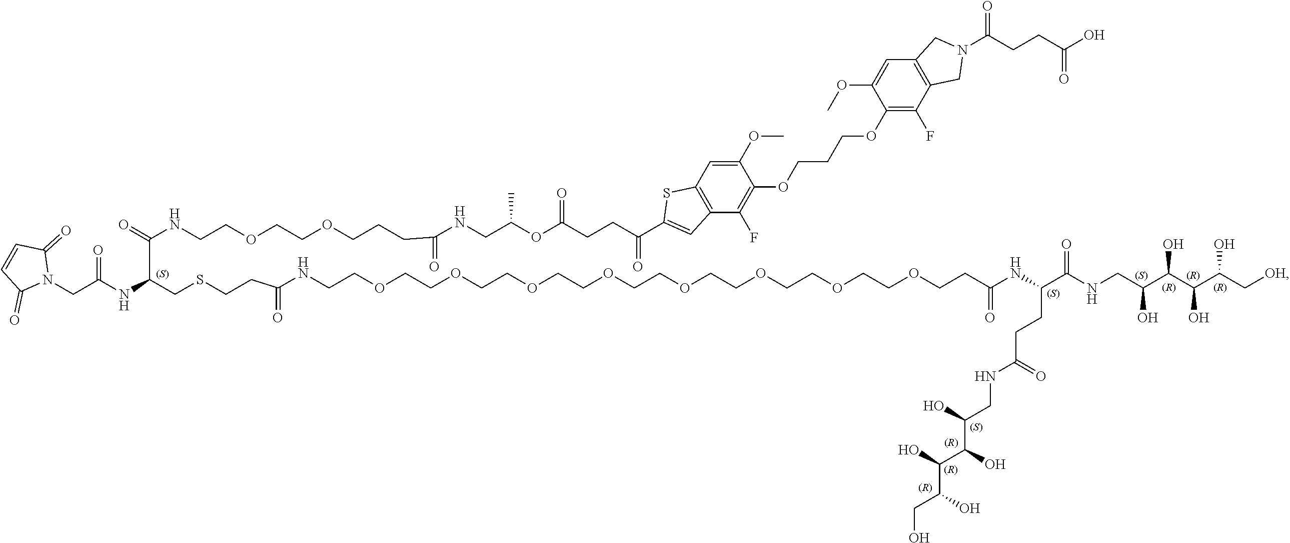

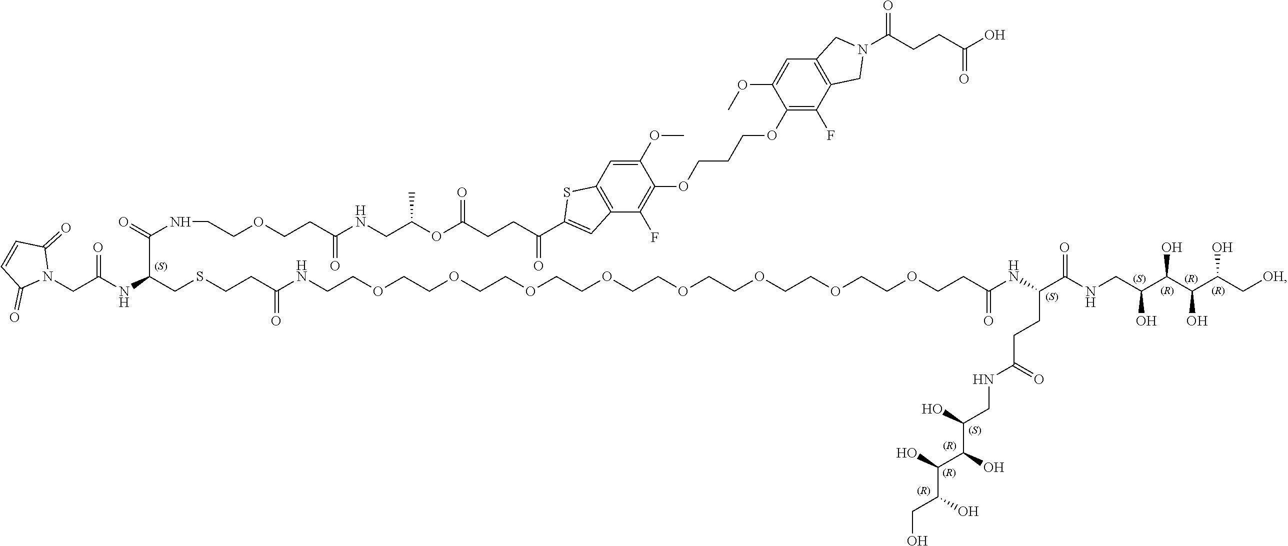

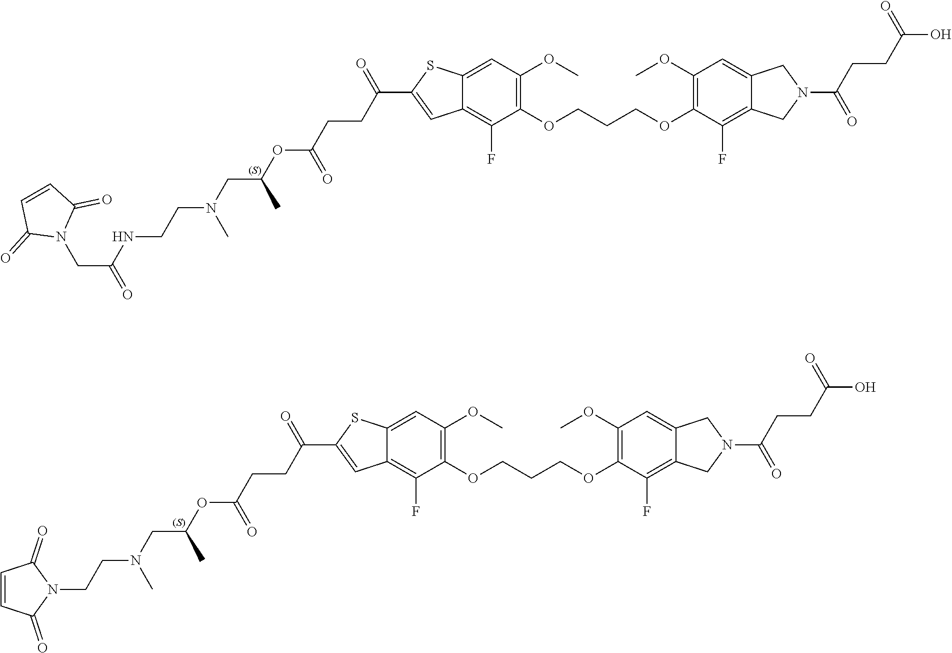

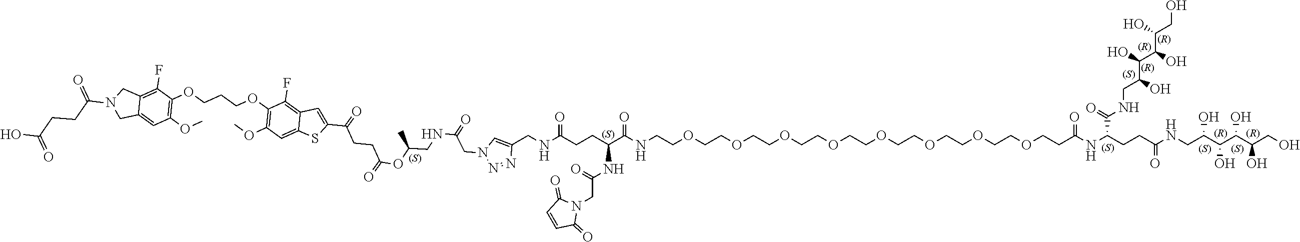

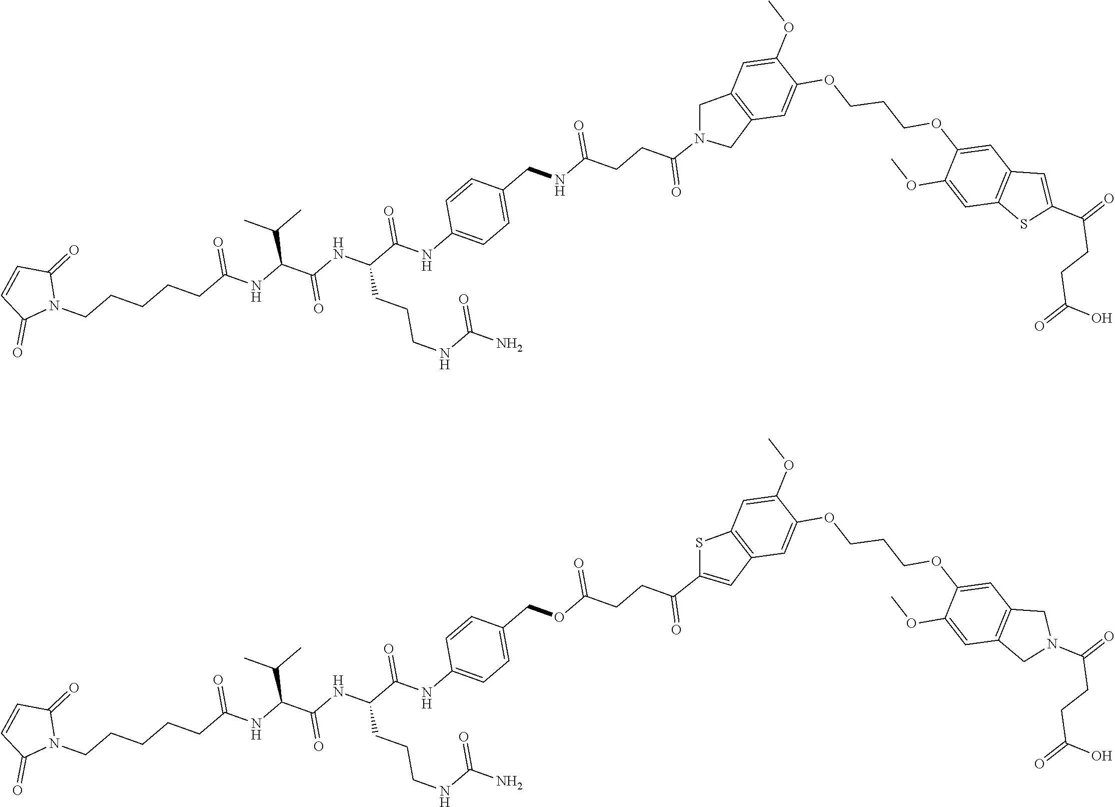

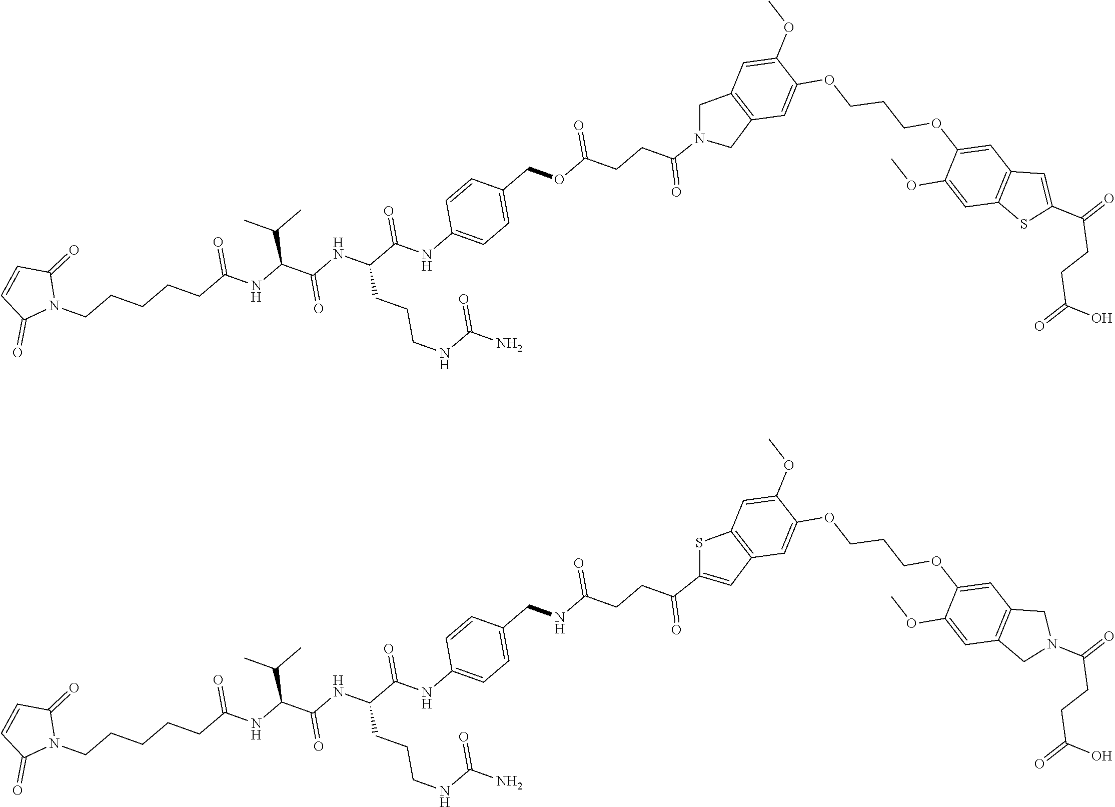

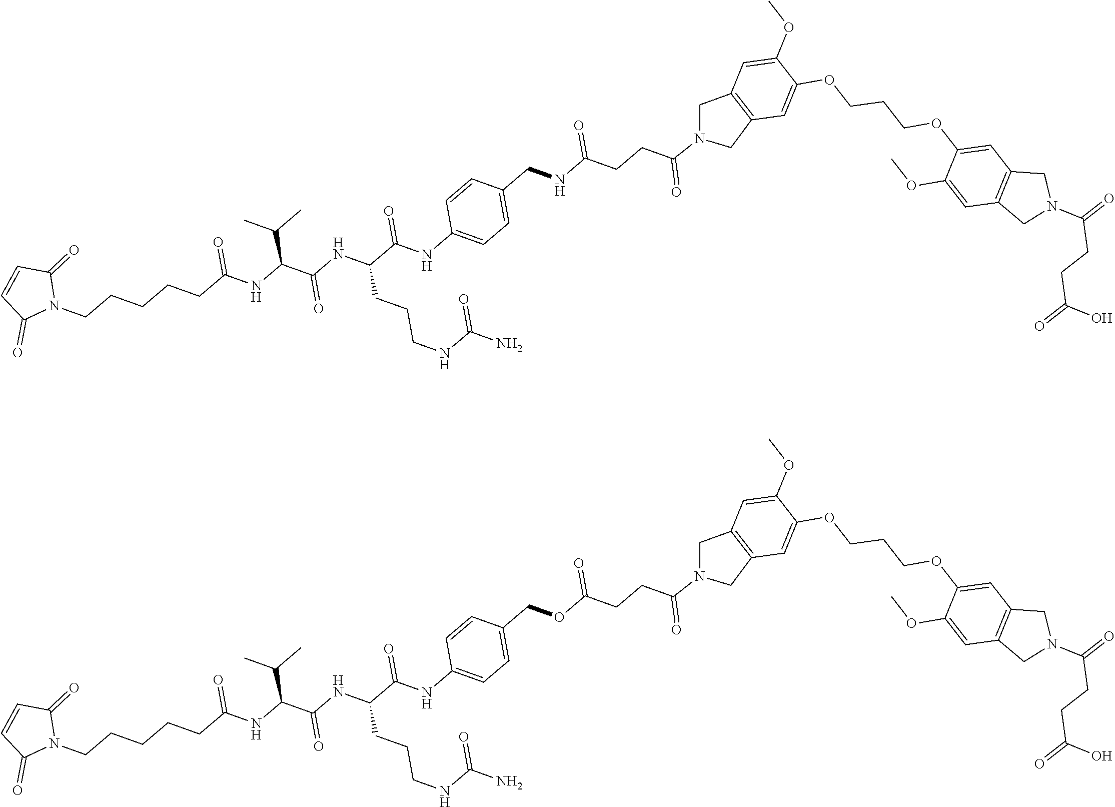

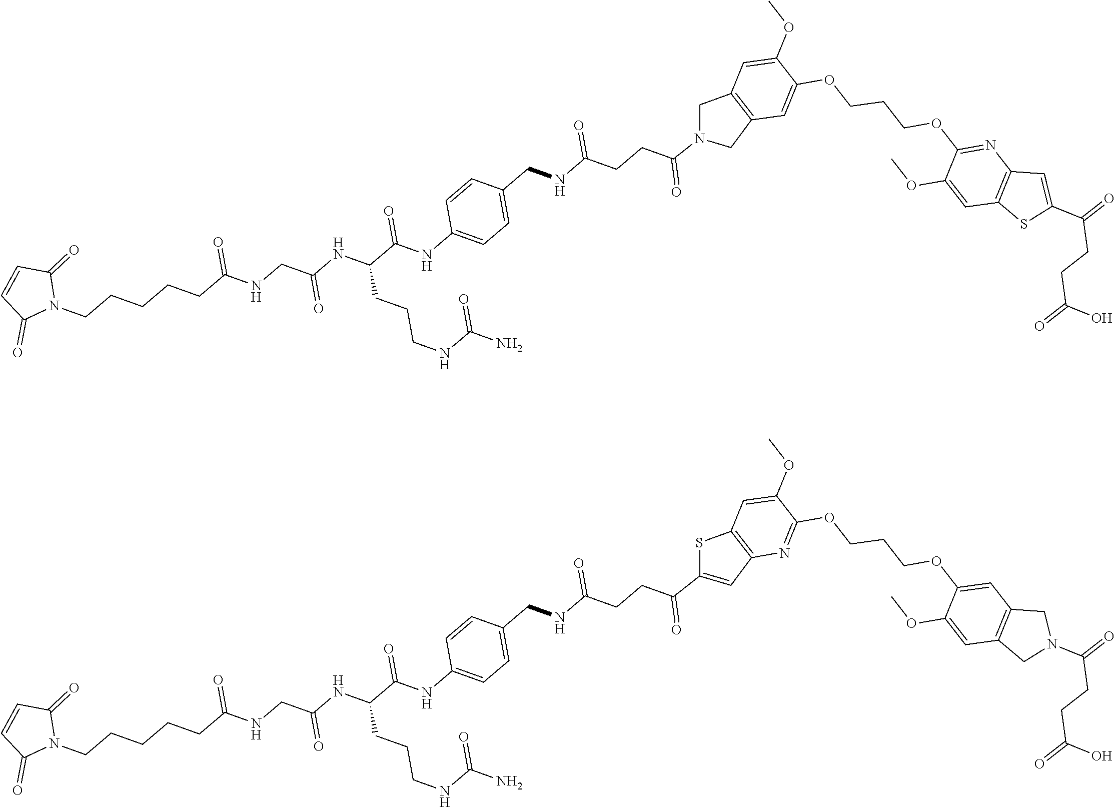

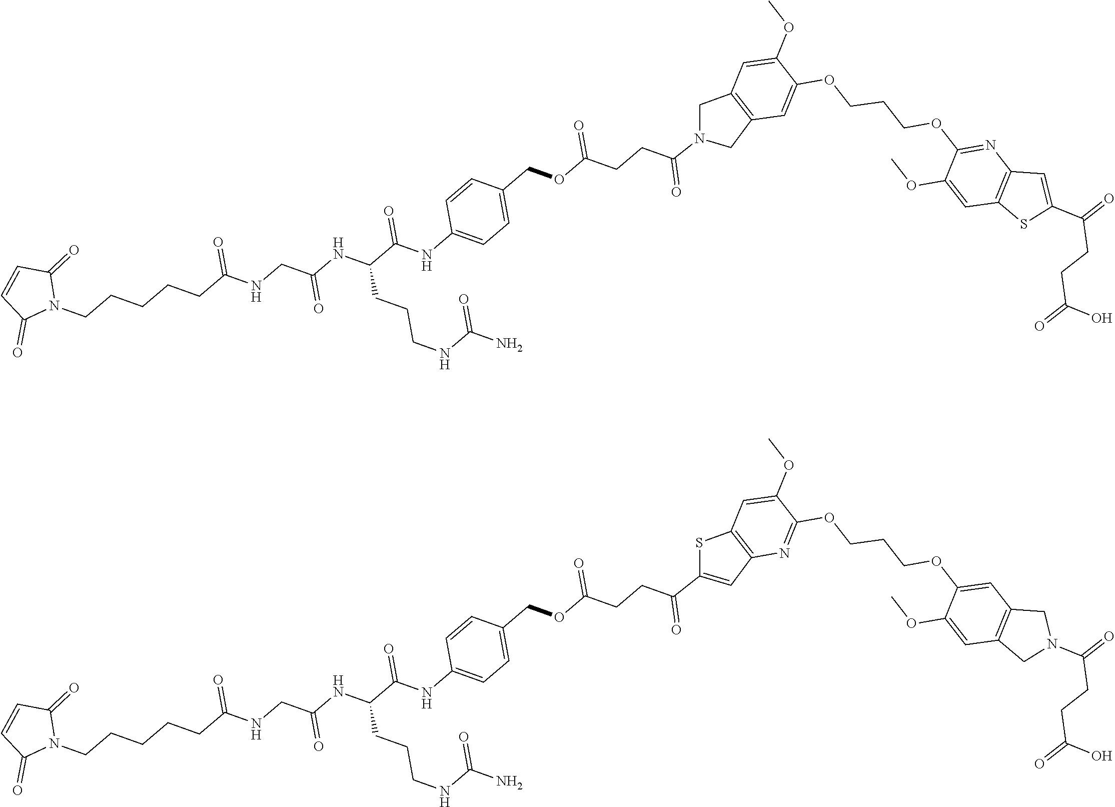

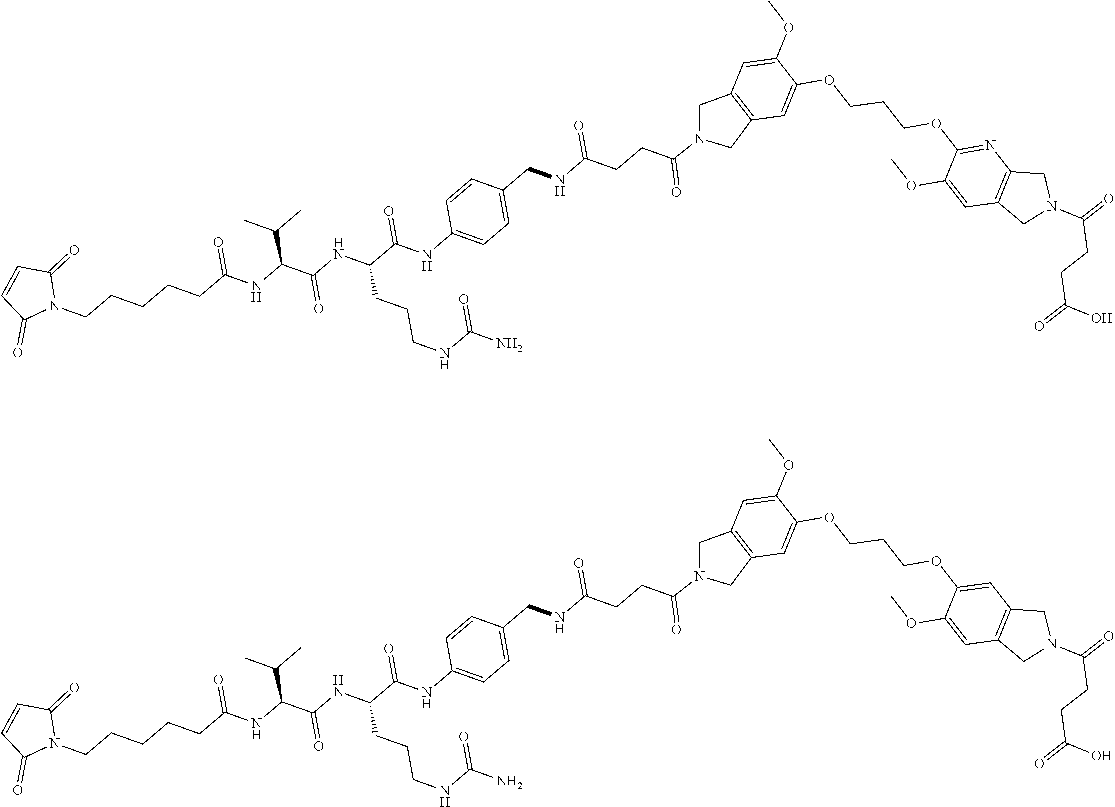

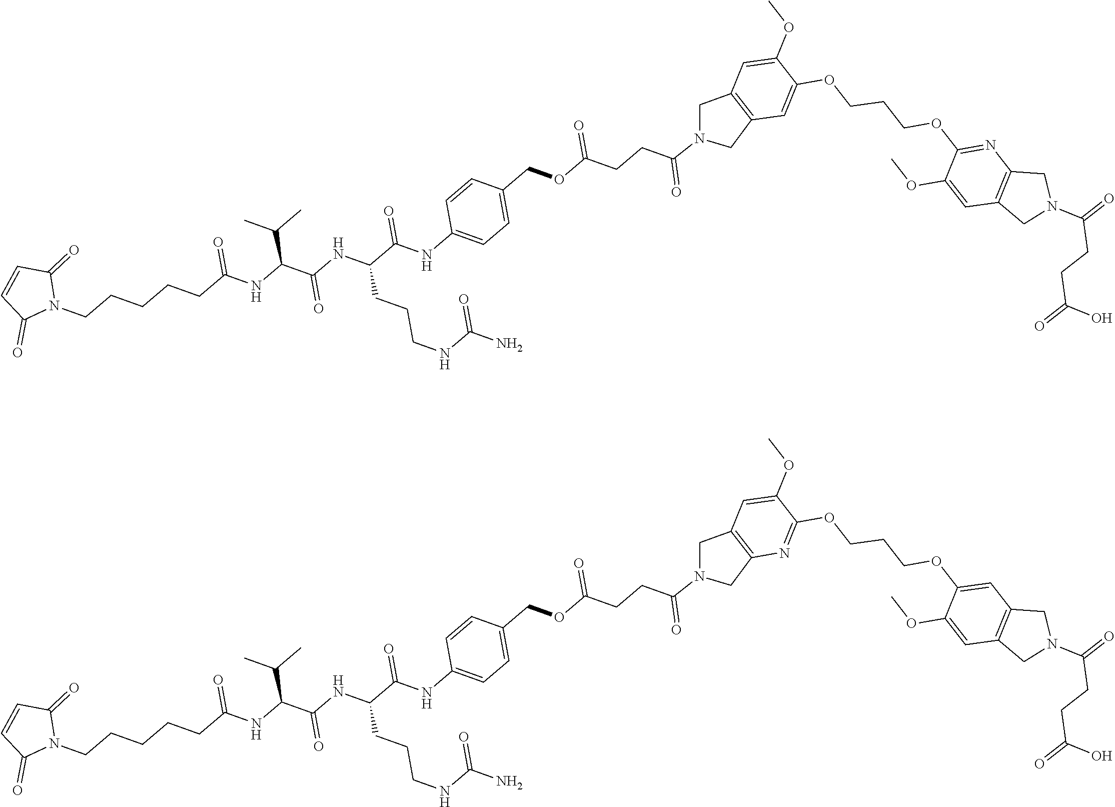

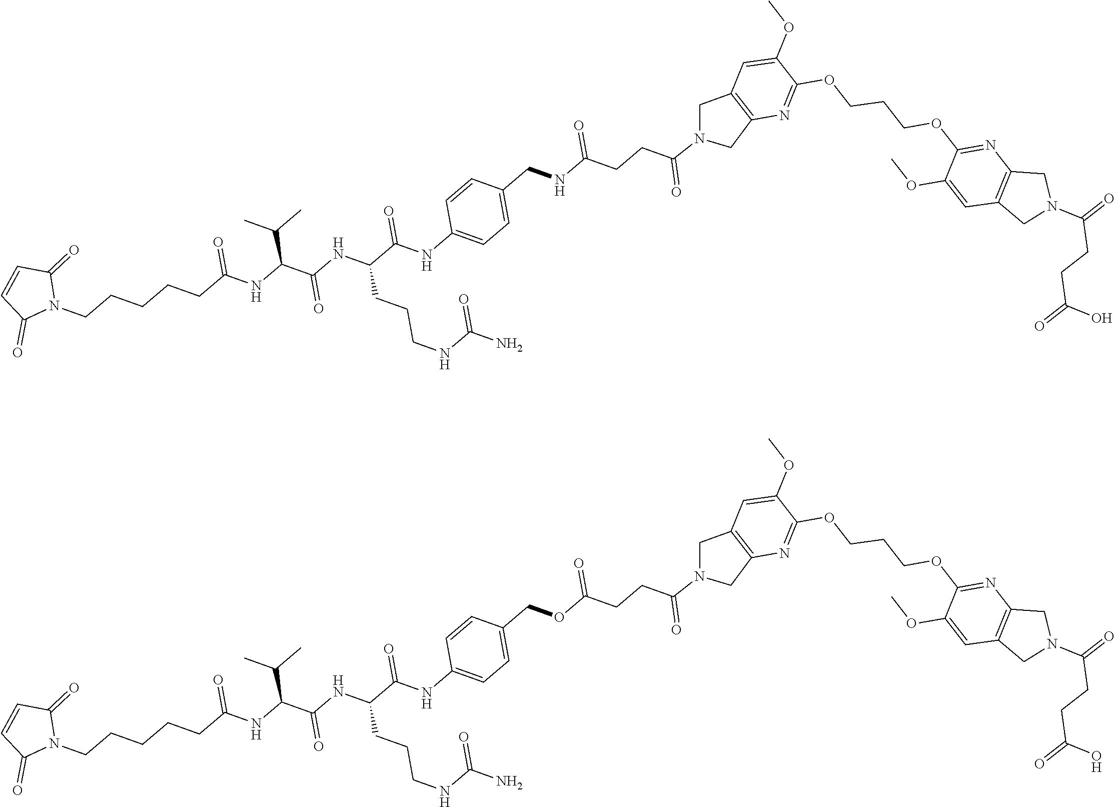

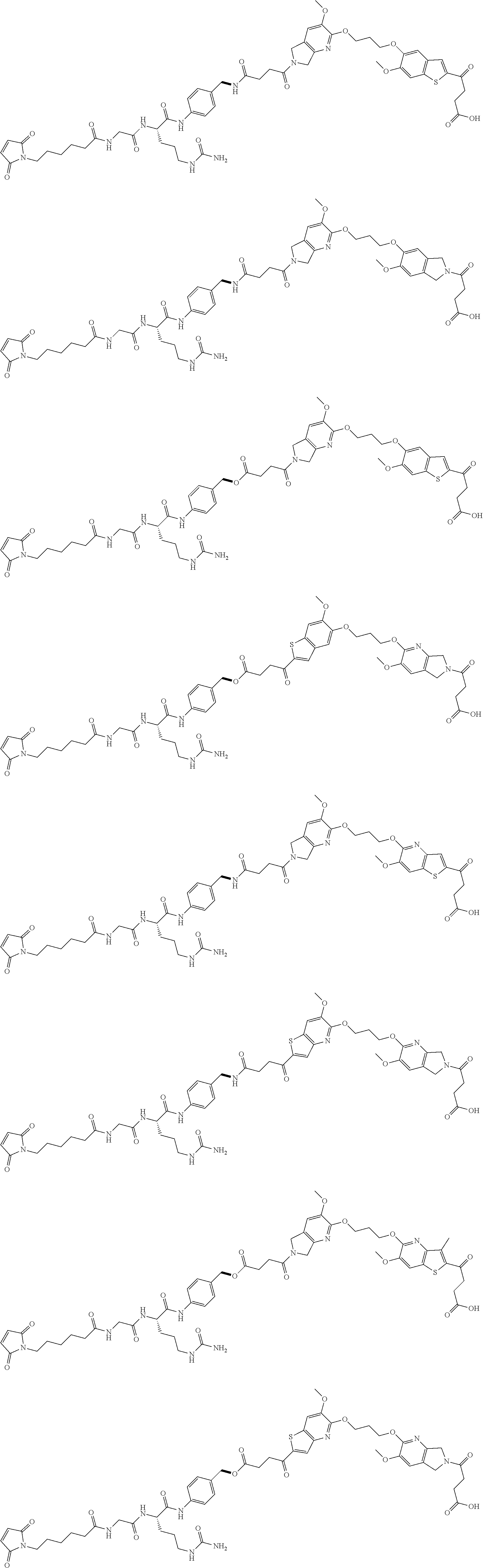

[chemical structure]

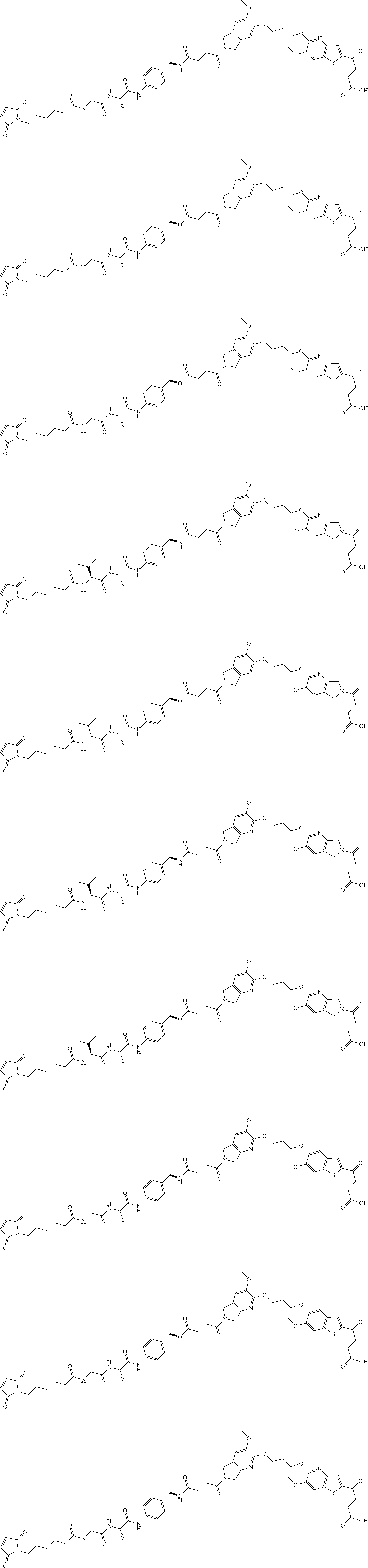

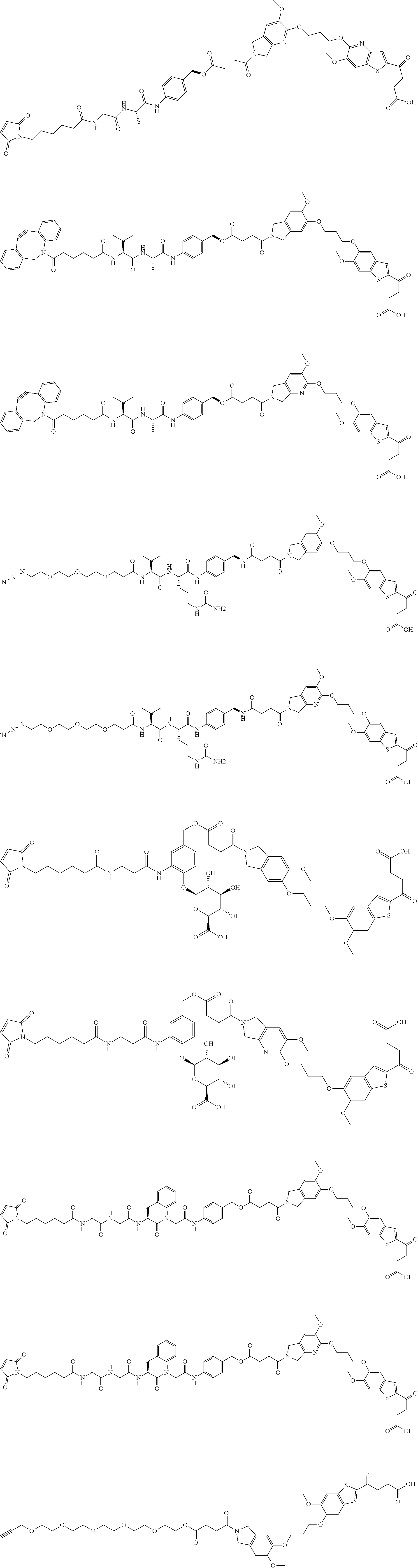

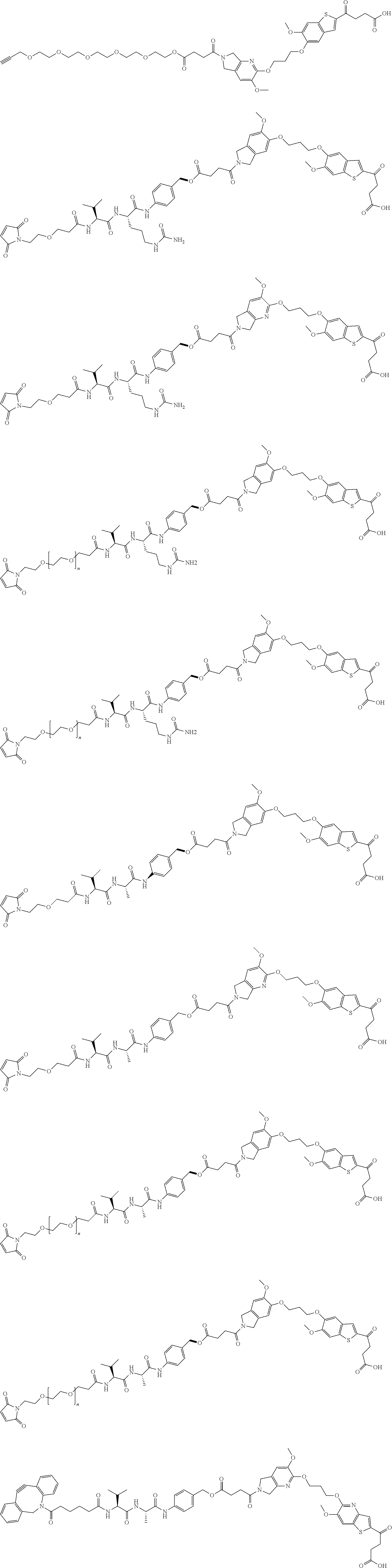

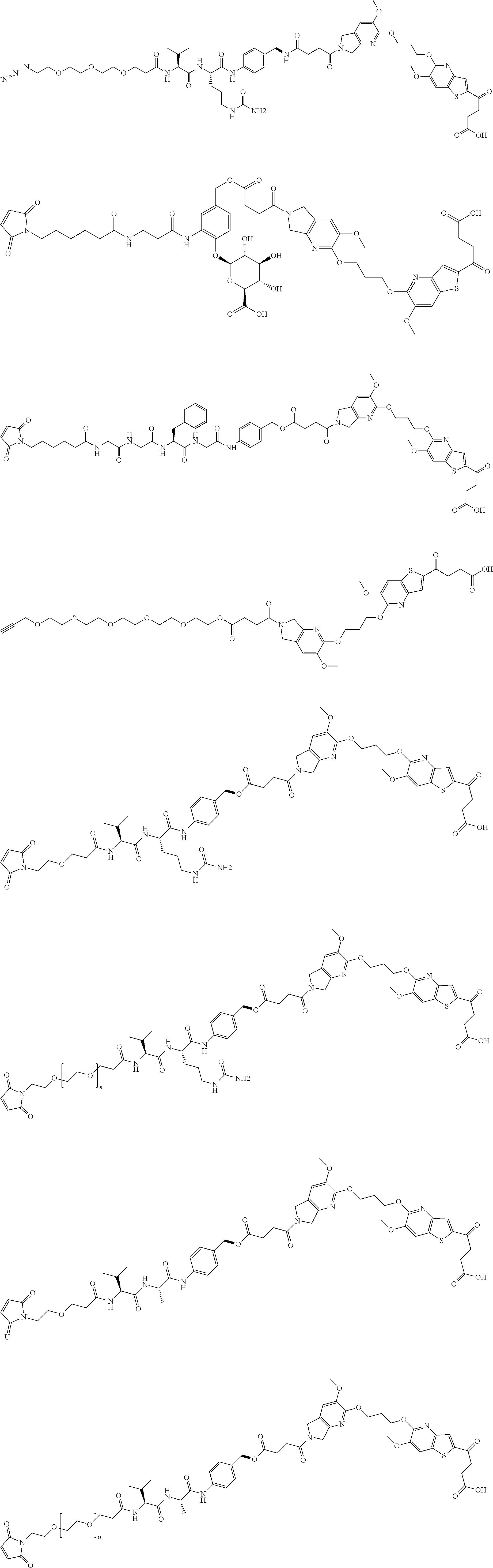

n is an integer ranging from 1 to 20, preferably 2 to 10, more preferably 2, 3, 4, 5, 6, 7, or 8.

In some embodiments, the compound-linker construct in the present invention is selected from the group consisting of:

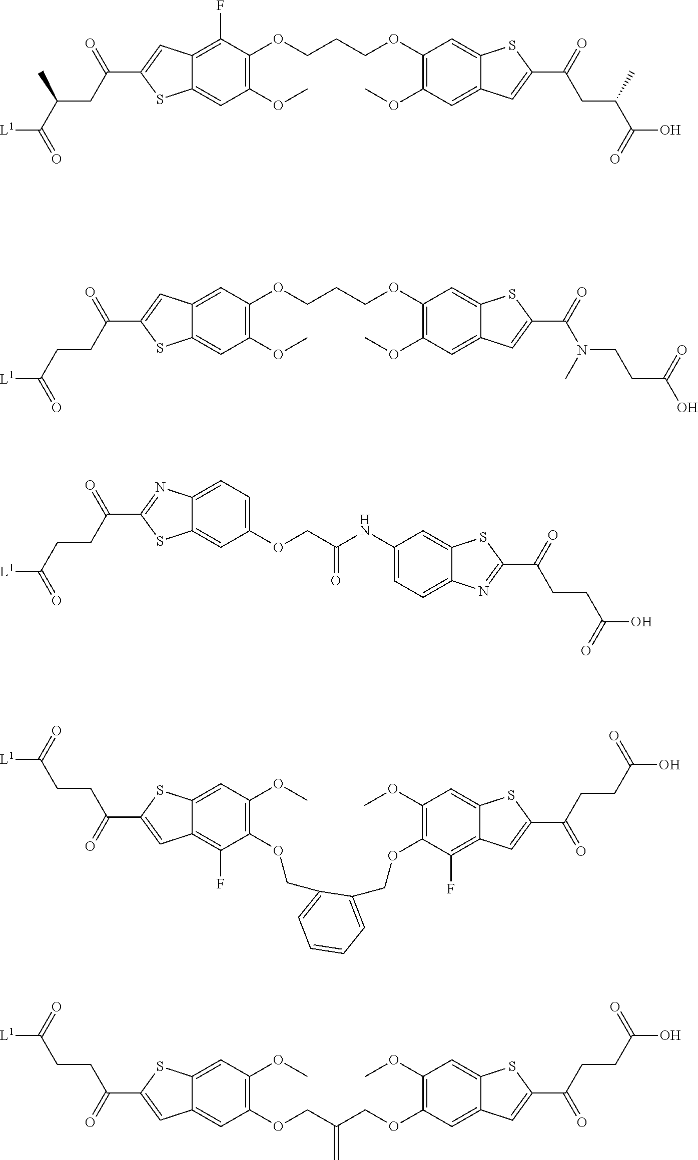

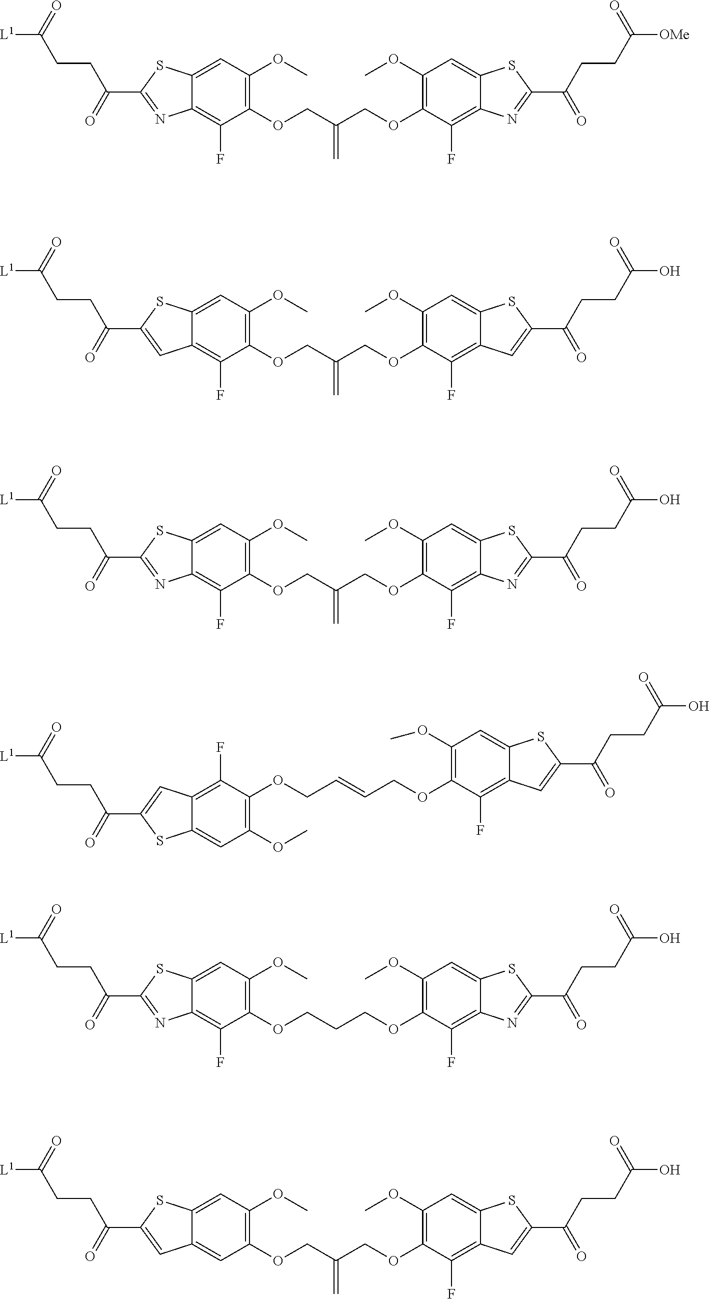

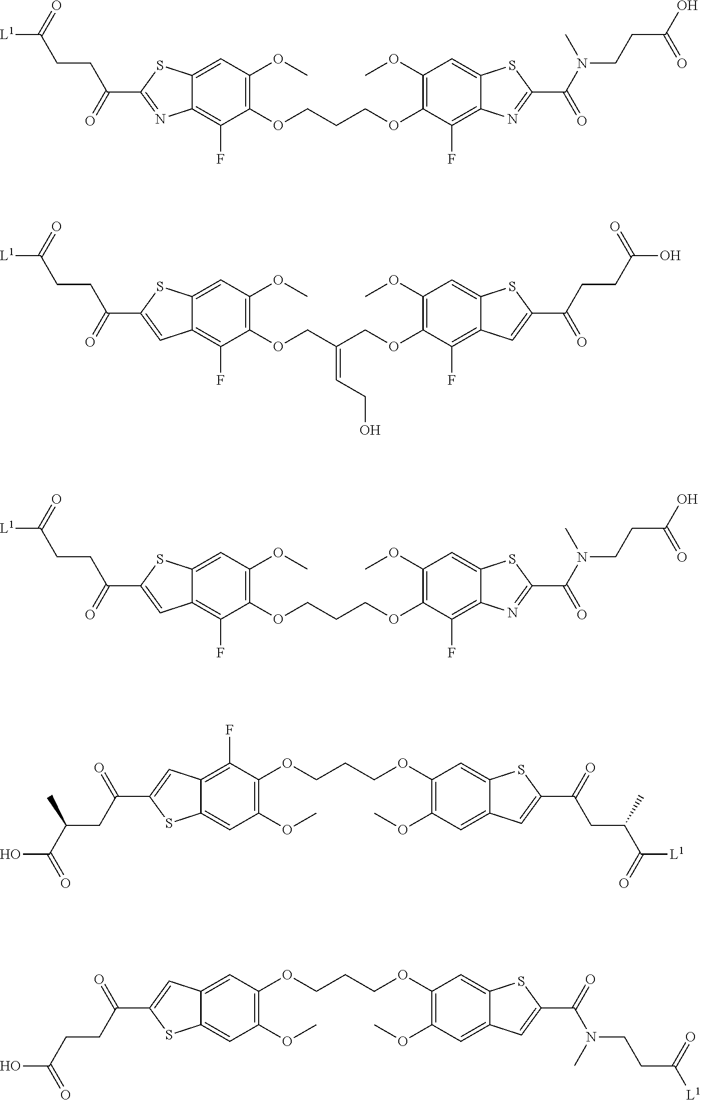

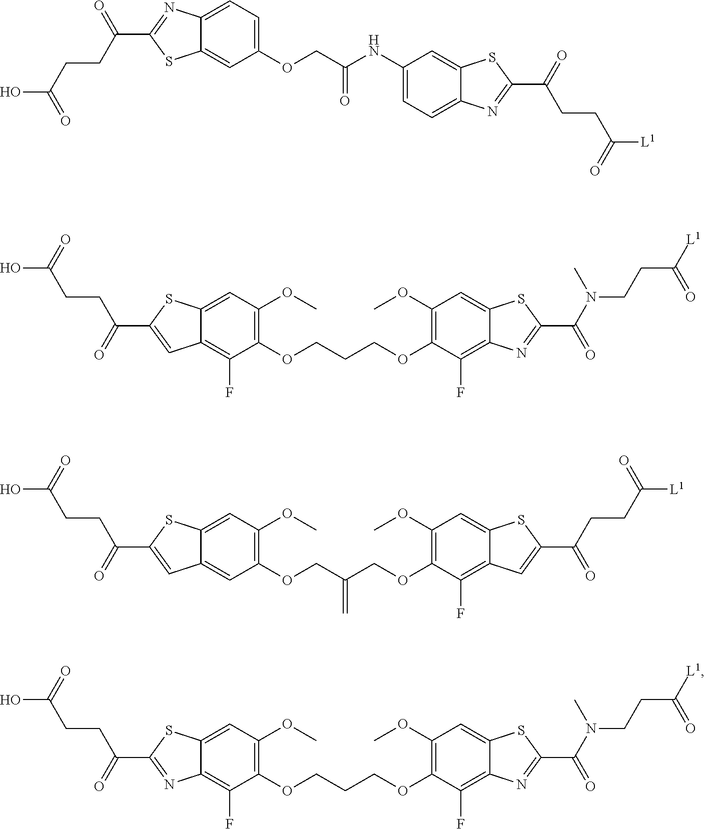

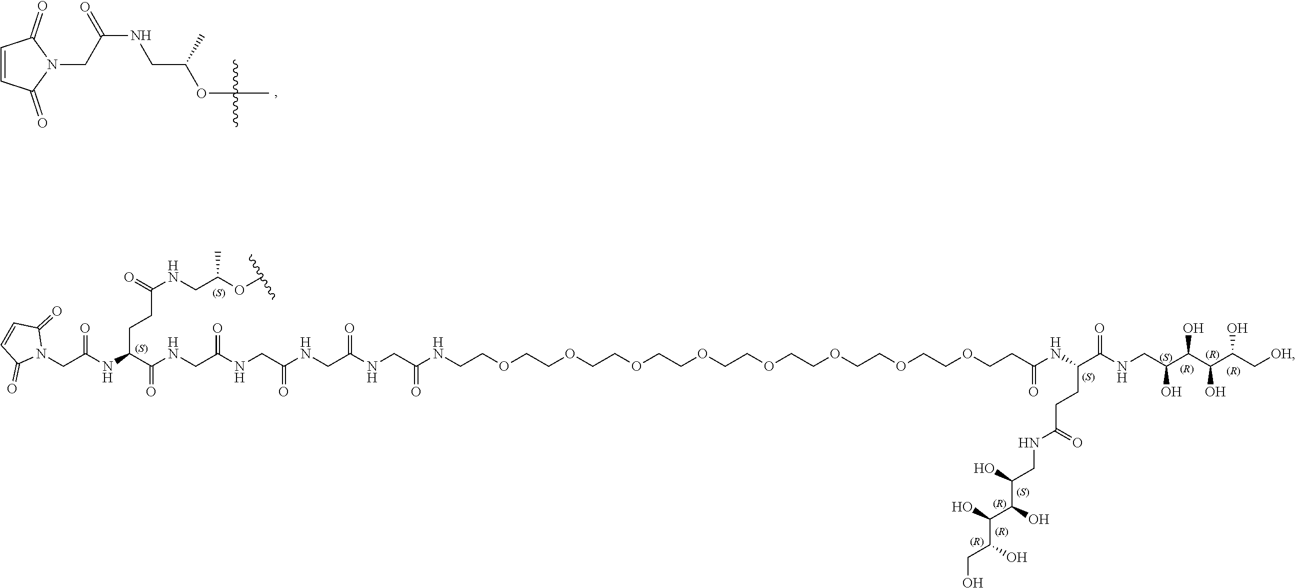

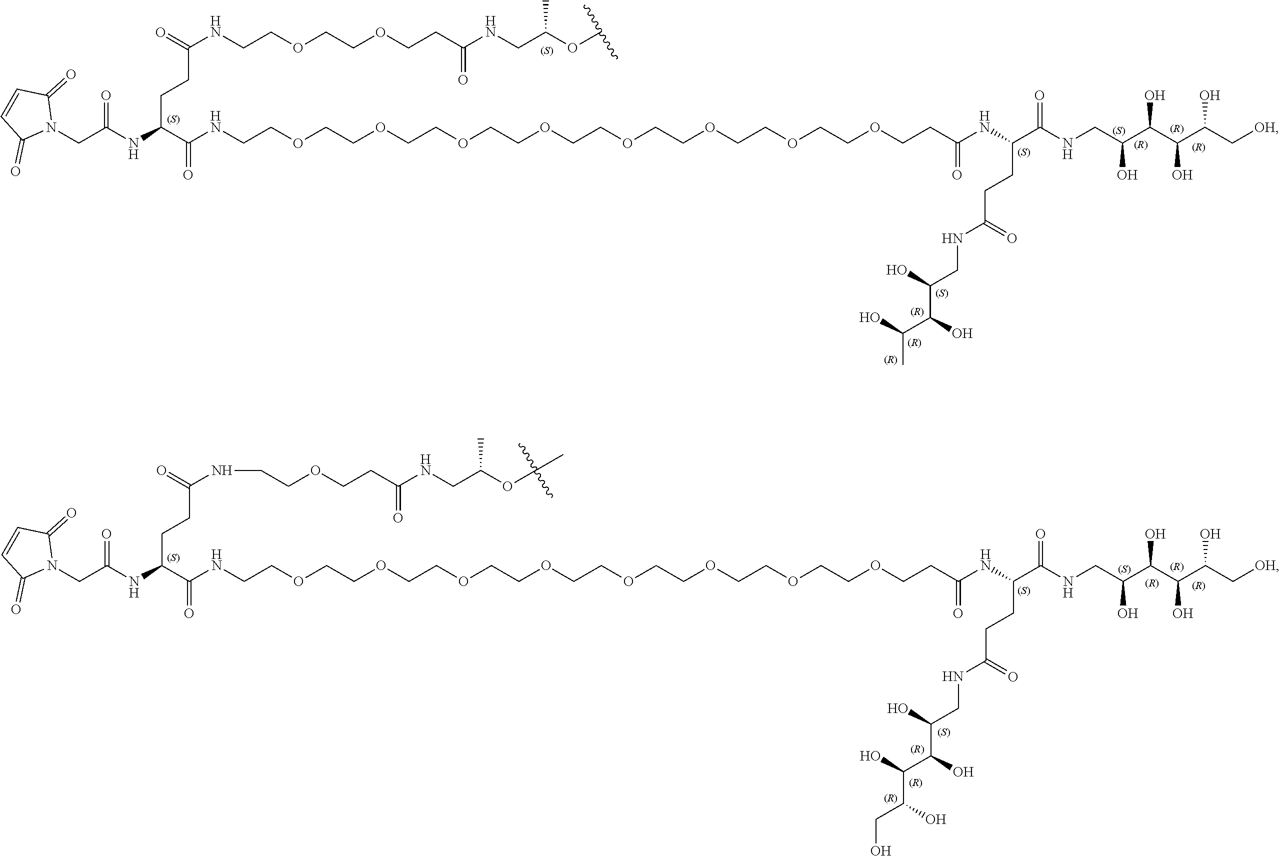

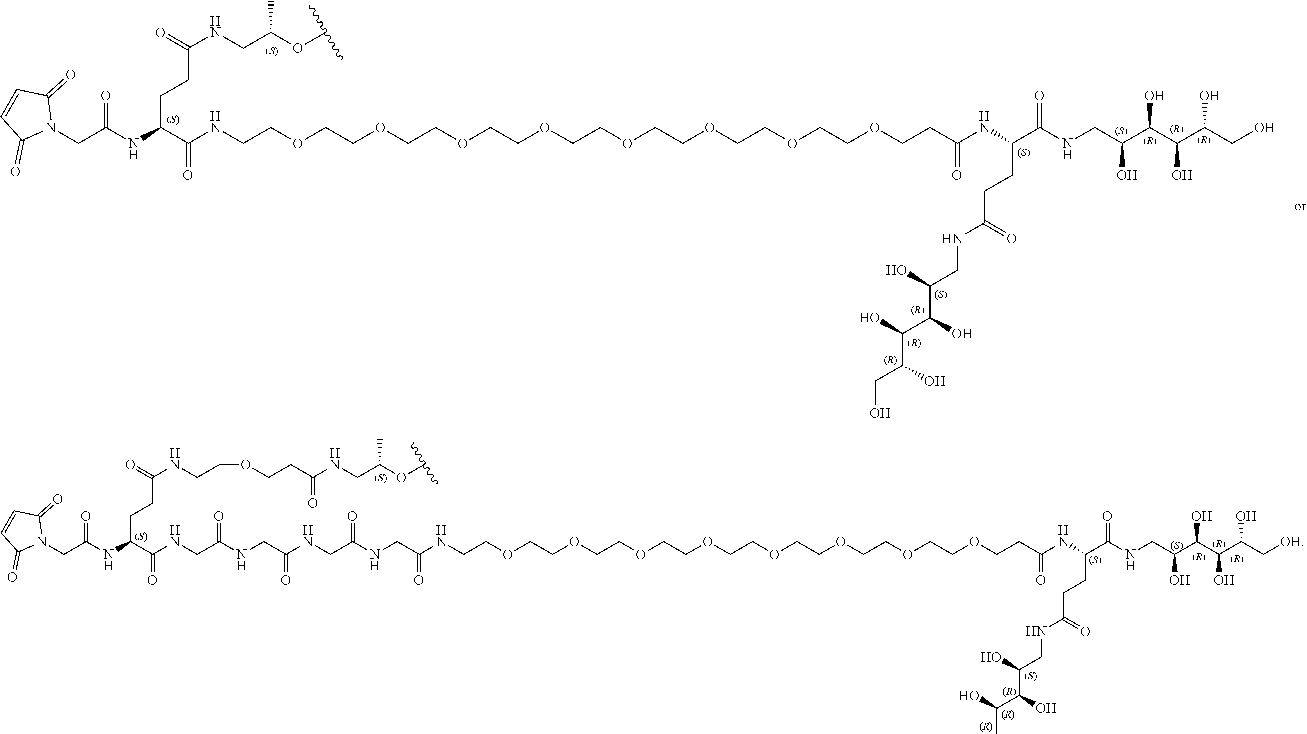

In some embodiments, the conjugate of the present disclosure, is selected from the group consisting of:

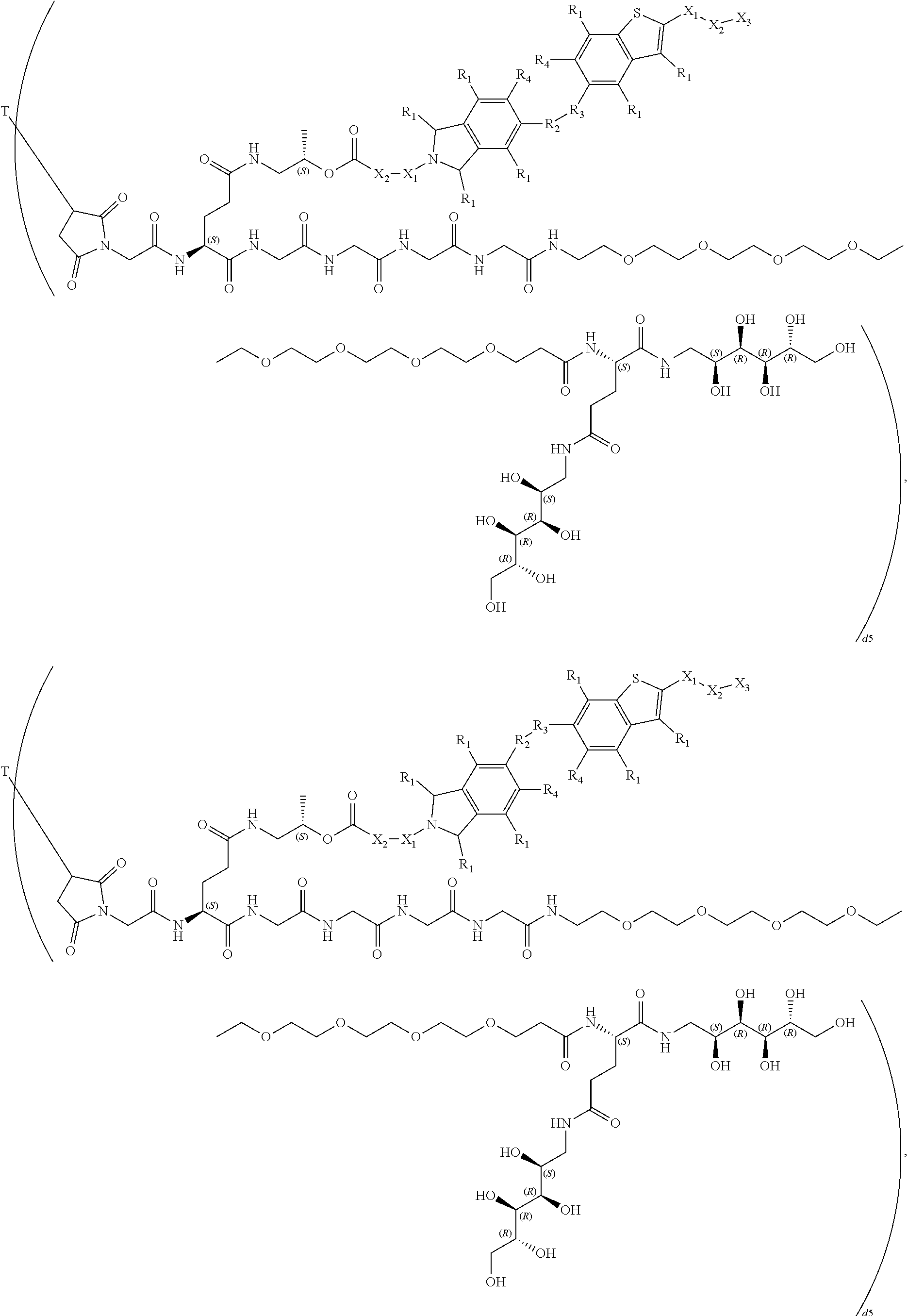

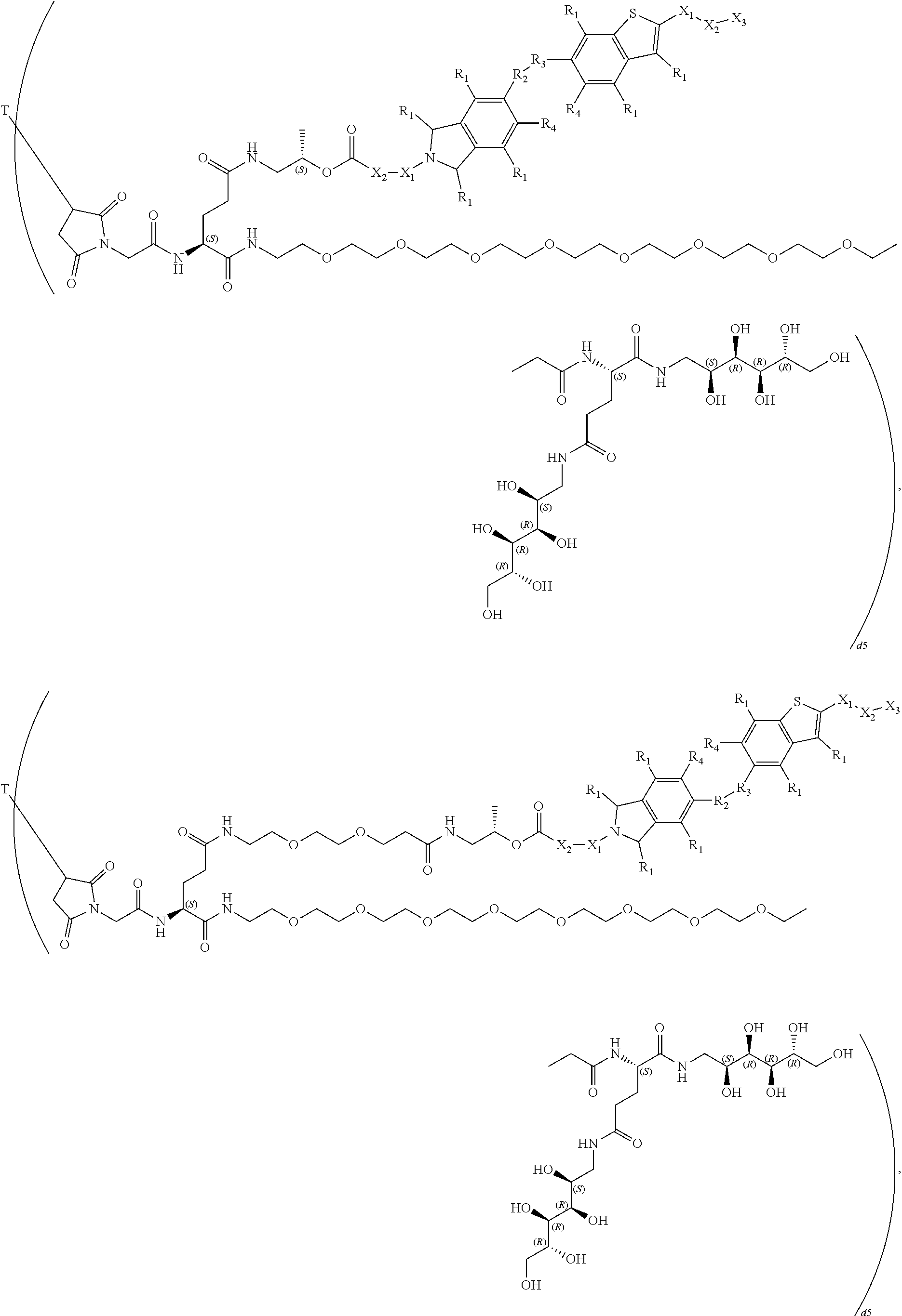

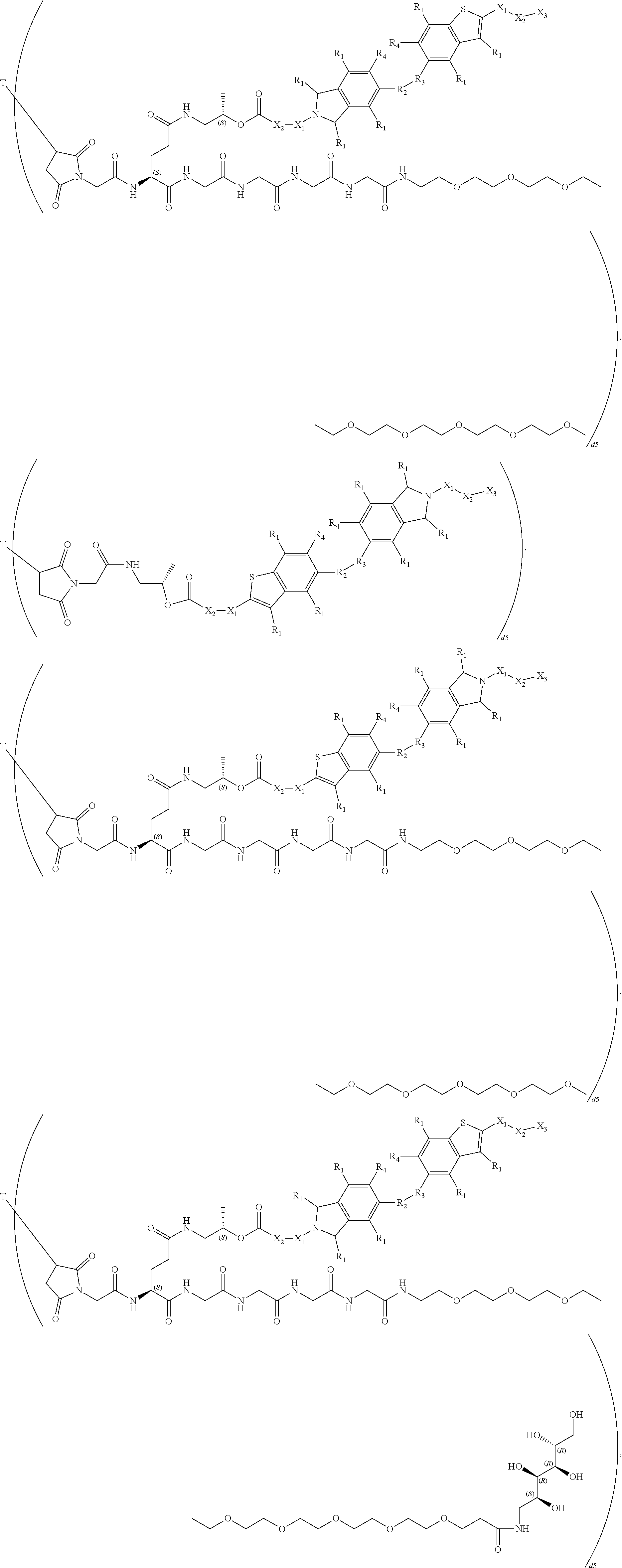

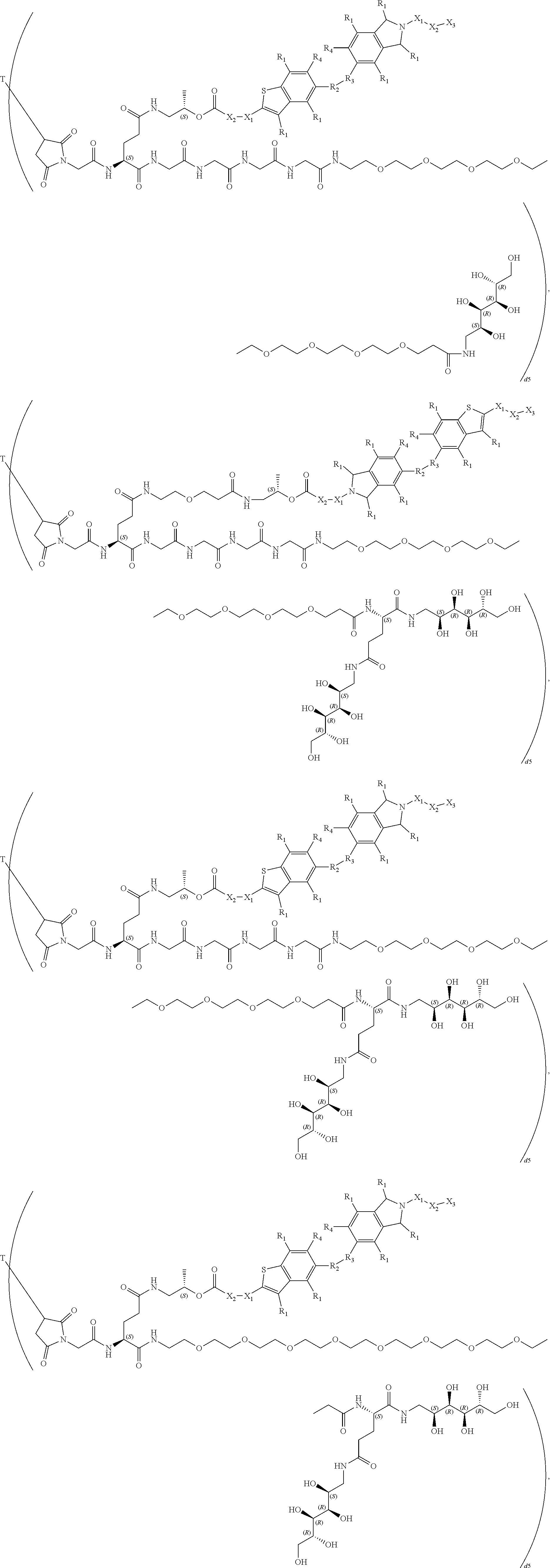

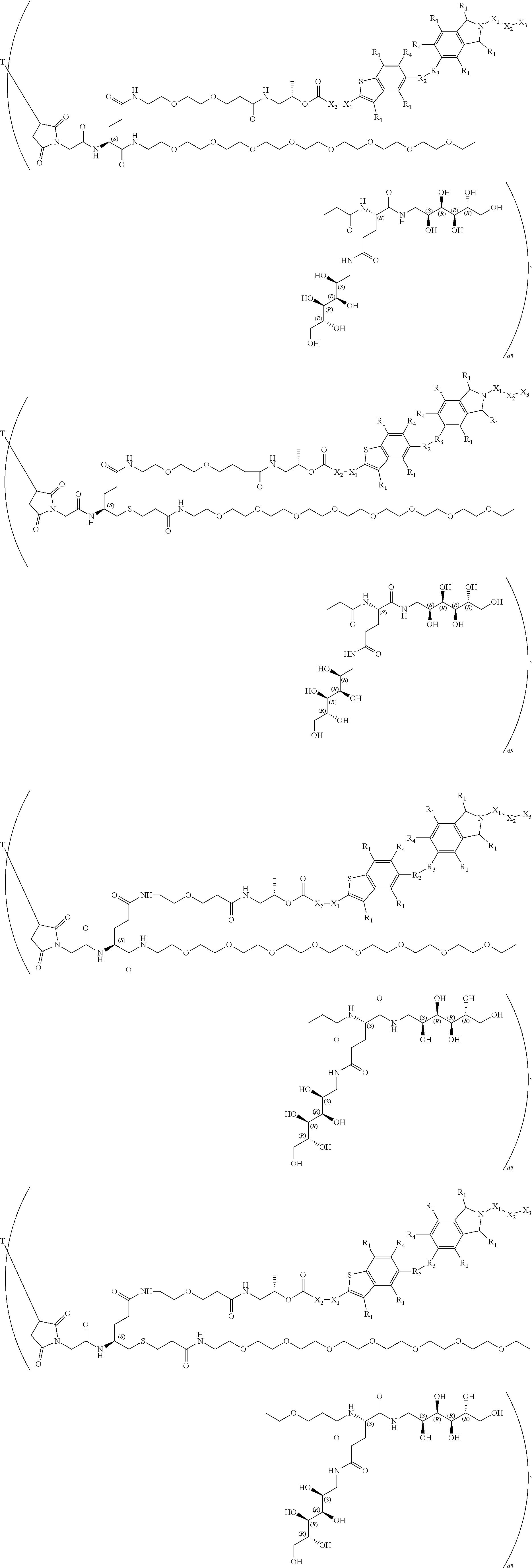

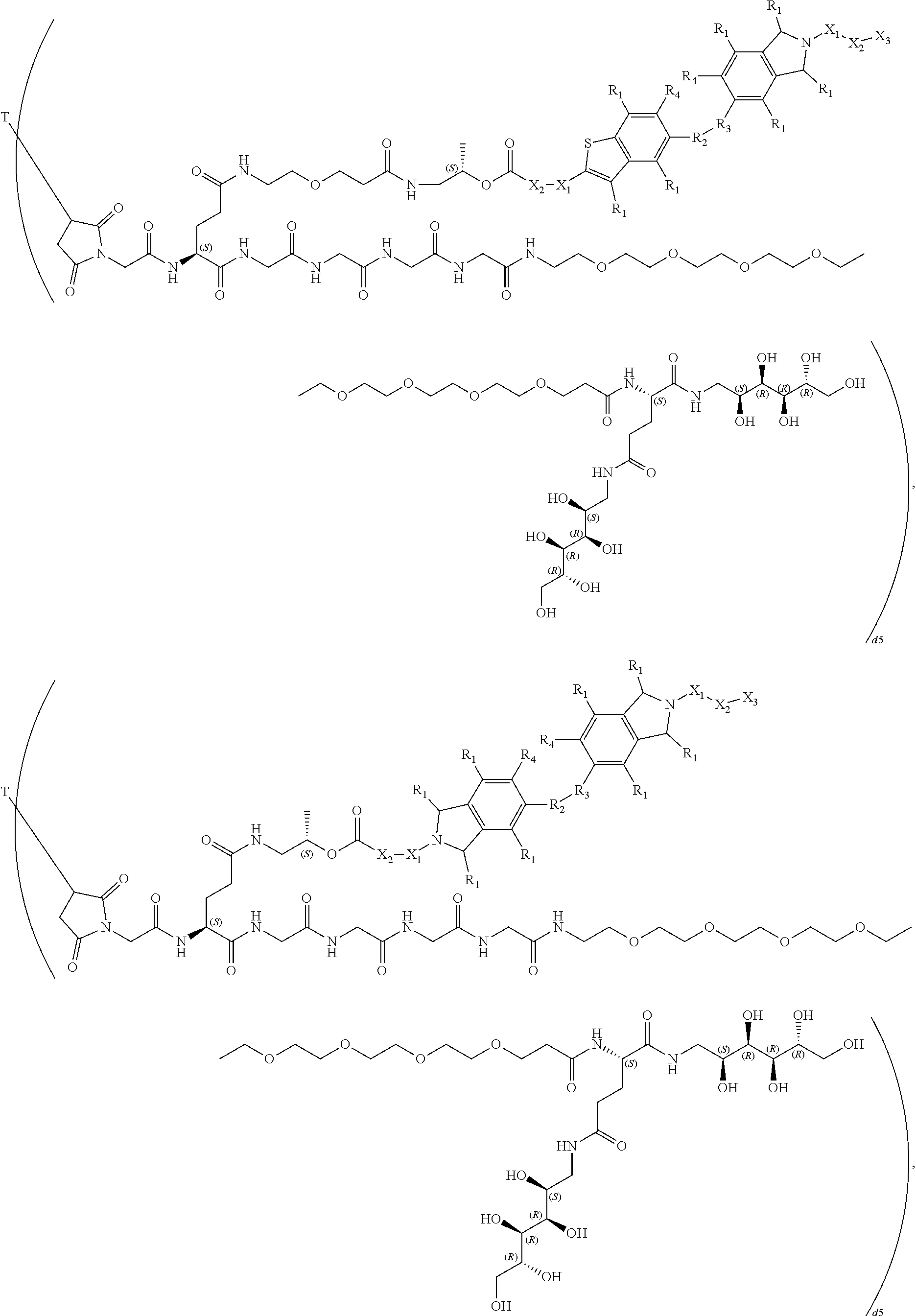

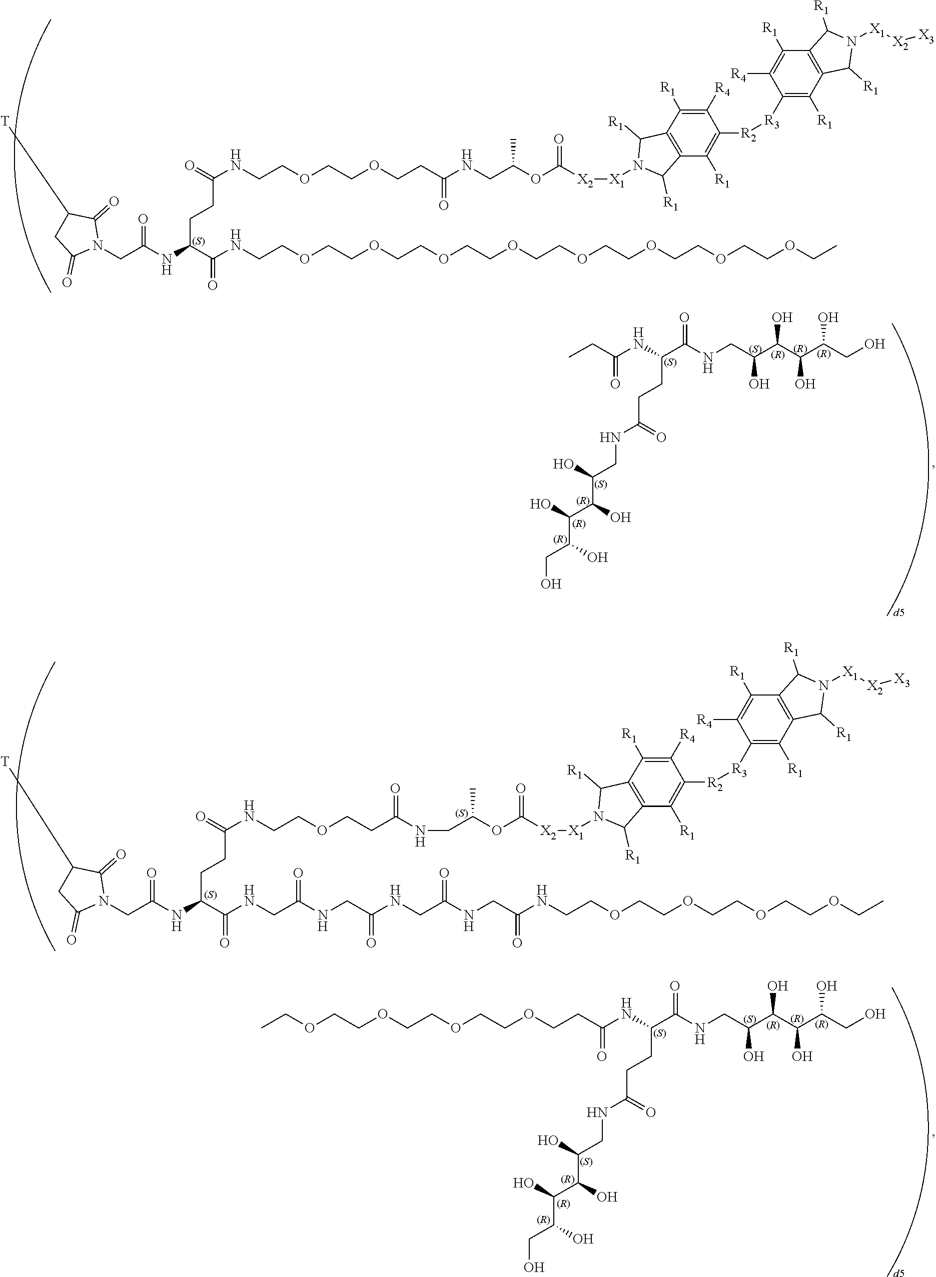

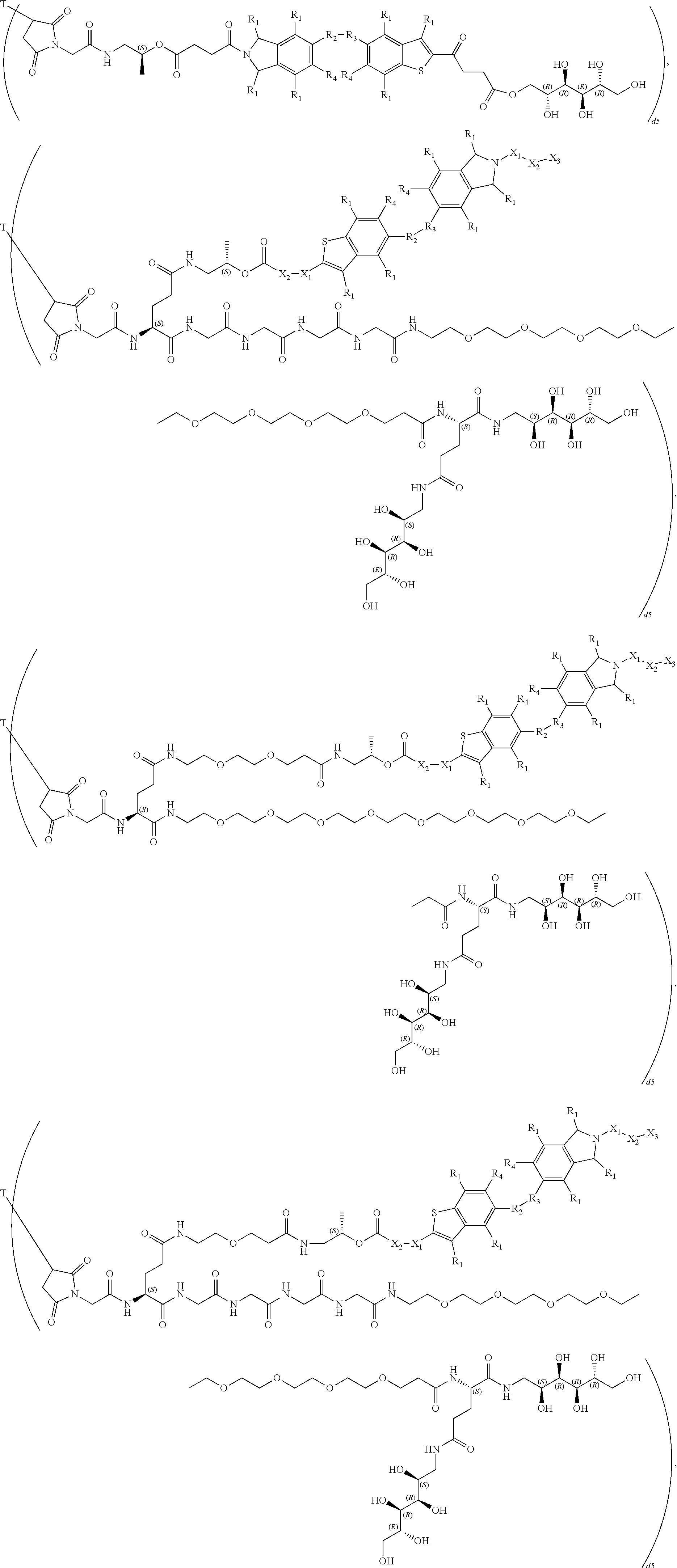

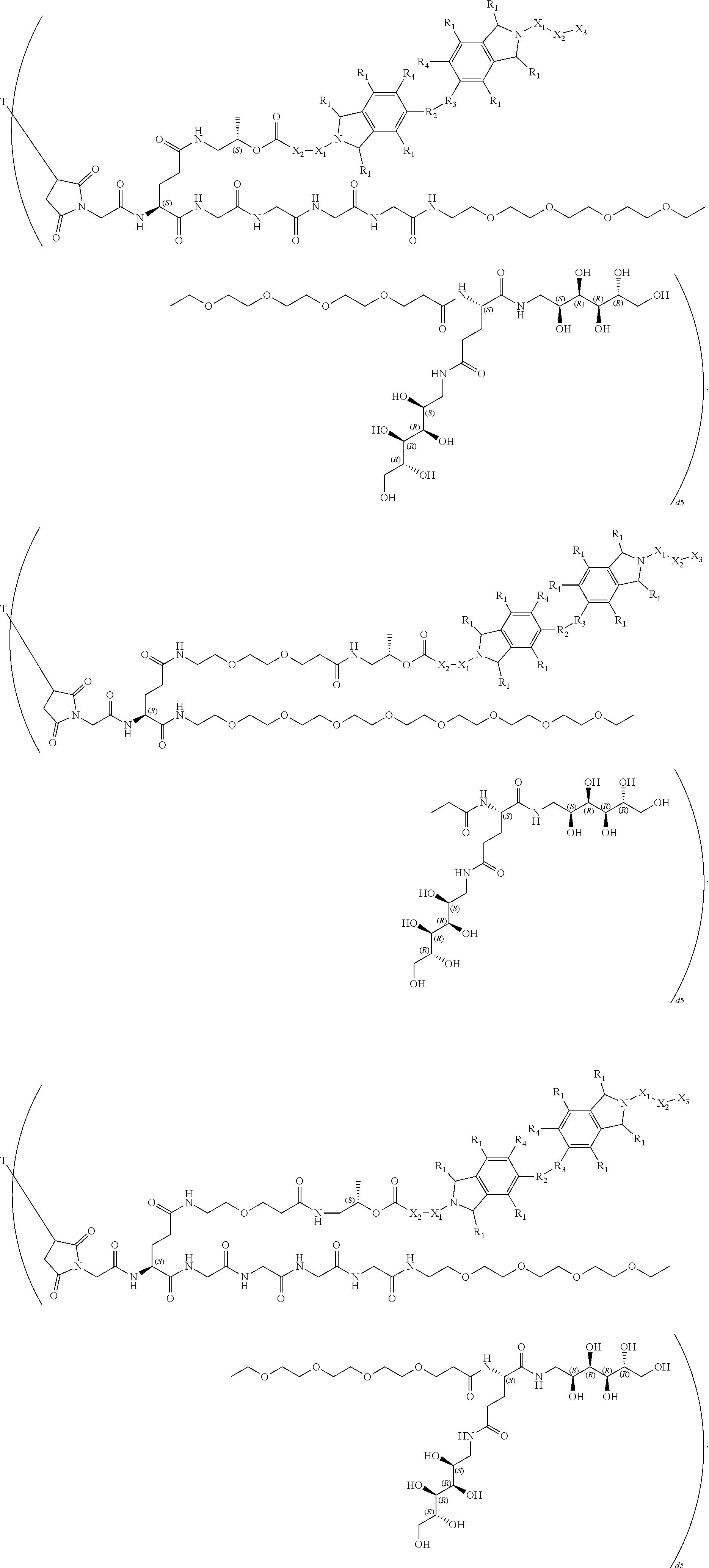

wherein each R¹, R², R³, R⁴, X₁, X₂ and X₃ is defined as above, and d5 is an integer ranging from 1 to 20, such as 6 to 10, such as 8.

In some embodiments, the conjugate of the present disclosure, is selected from the group consisting of:

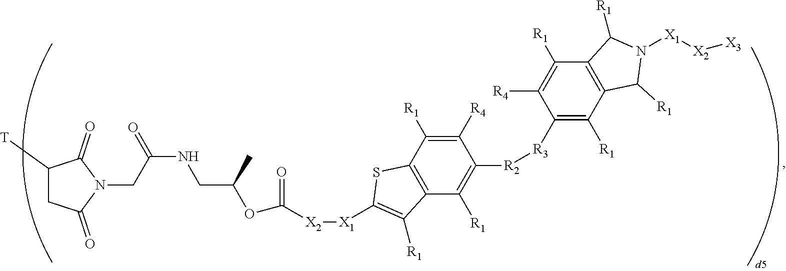

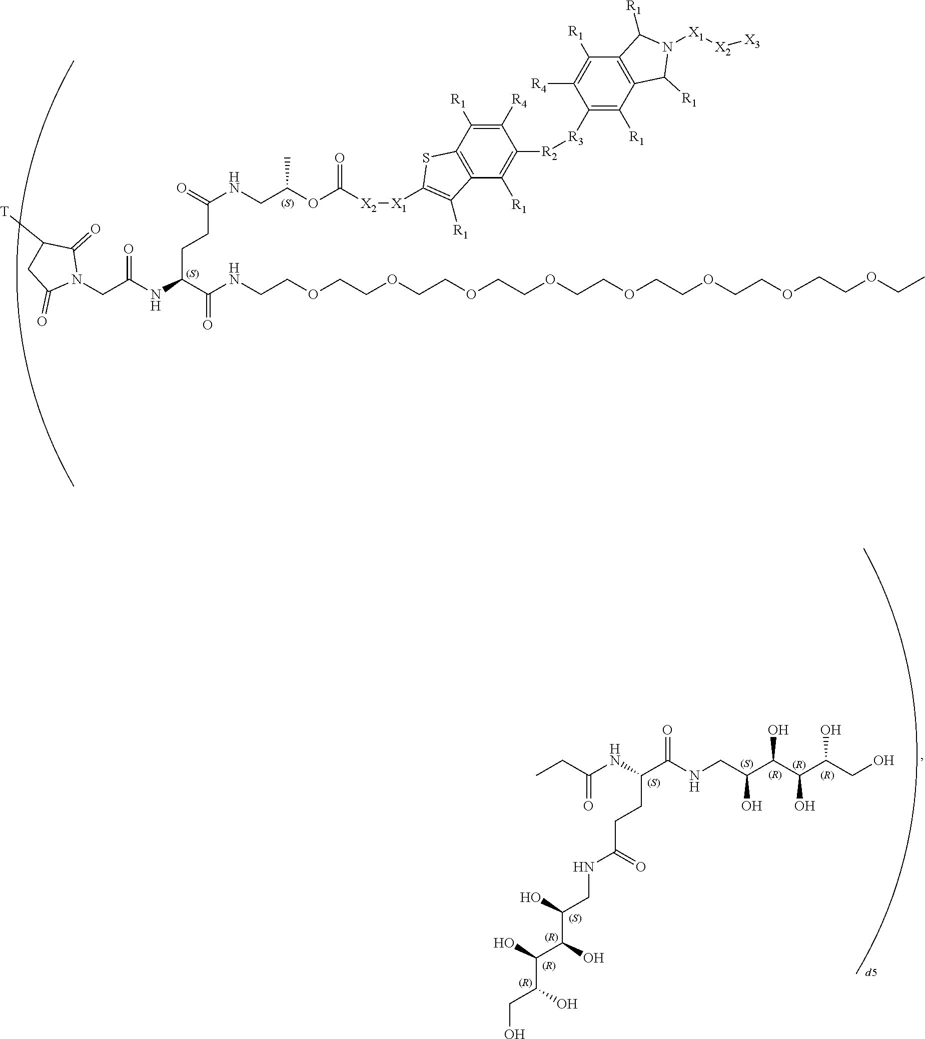

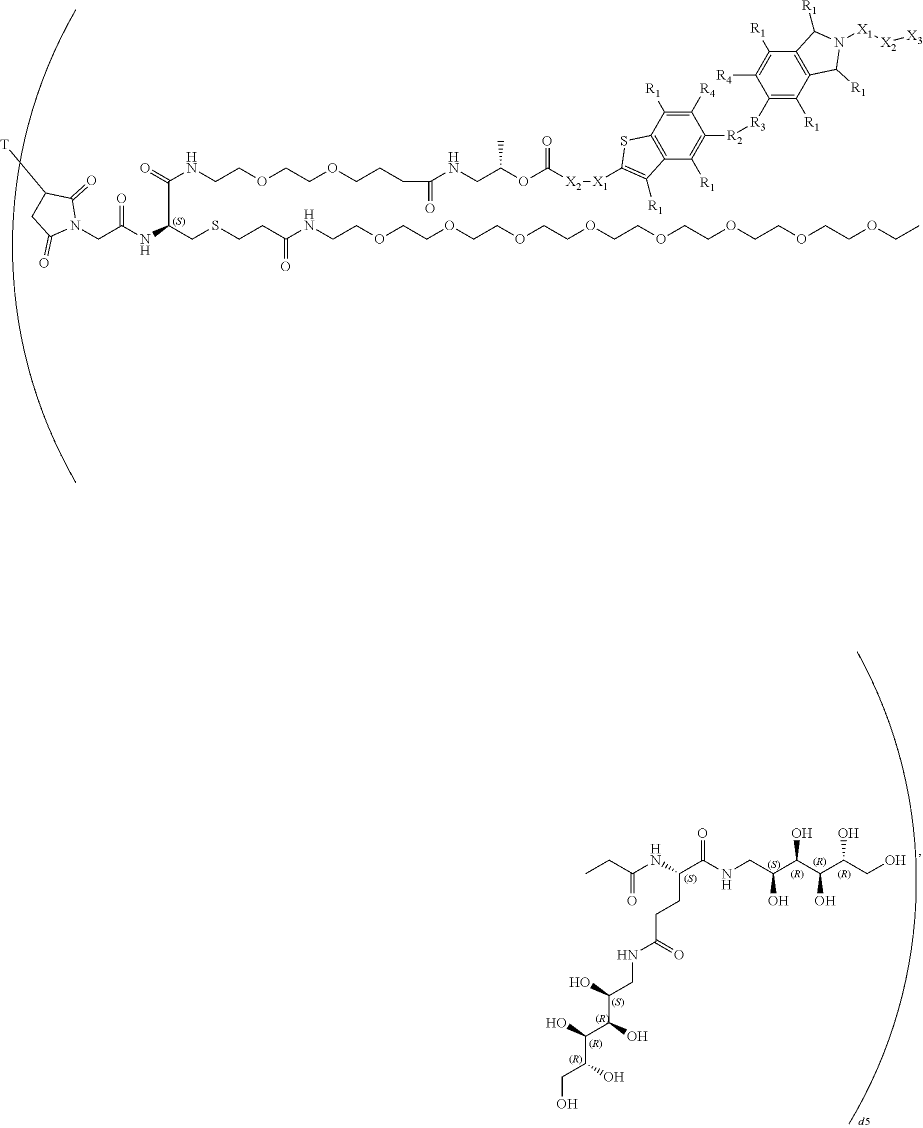

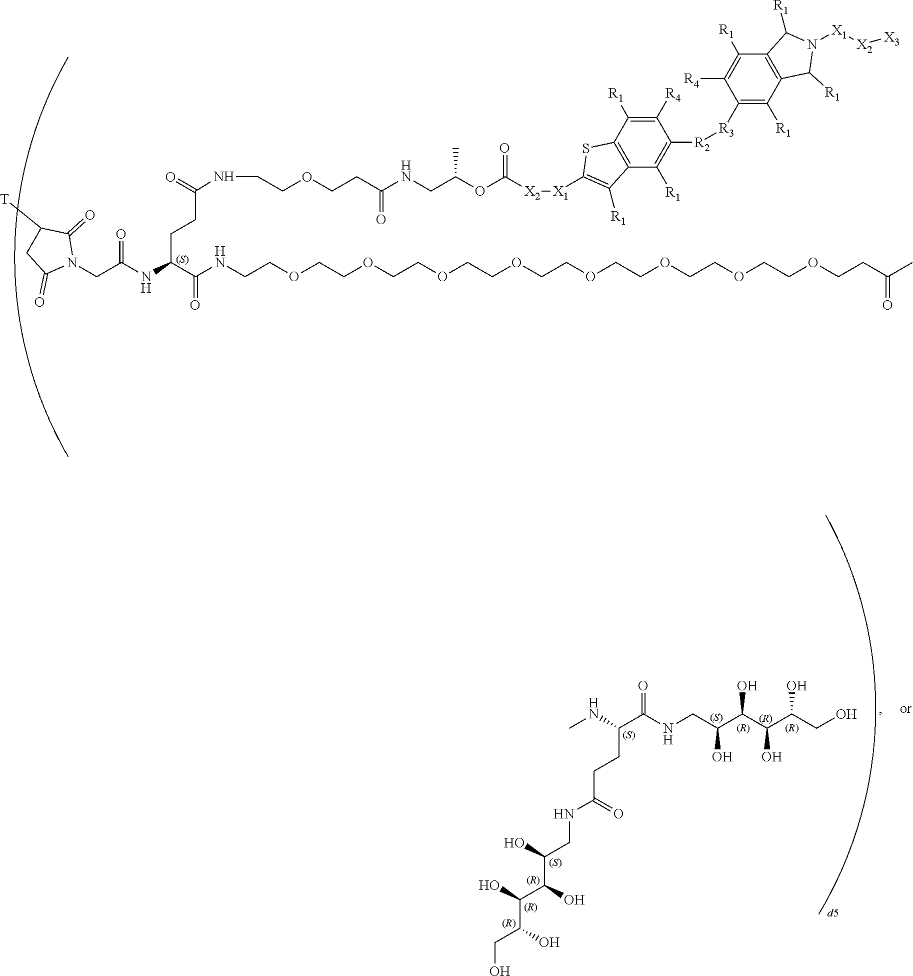

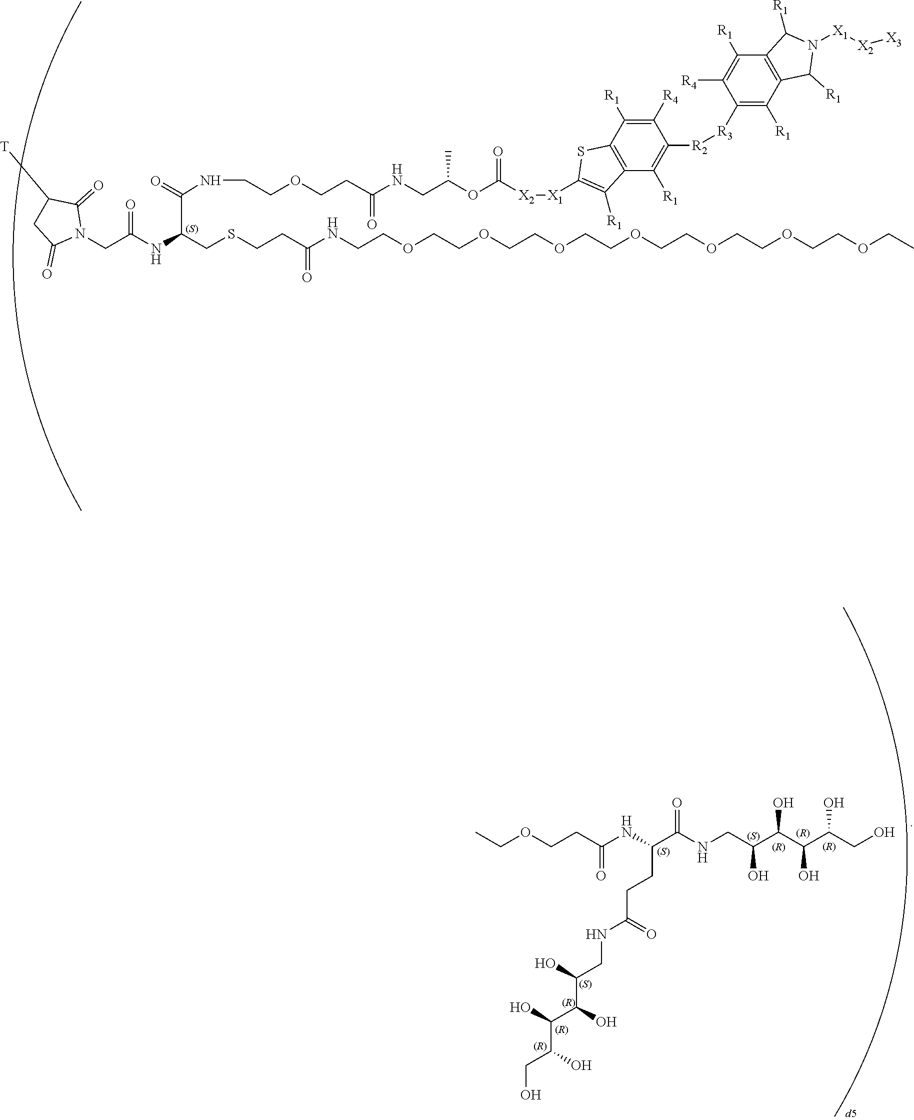

wherein each R₁, R₂, R₃, R⁴, X₁, X₂ and X₃ is defined as above, and d5 is an integer ranging from Ito 20, such as 6 to 10, such as 8.

In some embodiments, the conjugate of the present disclosure is selected from the group consisting of:

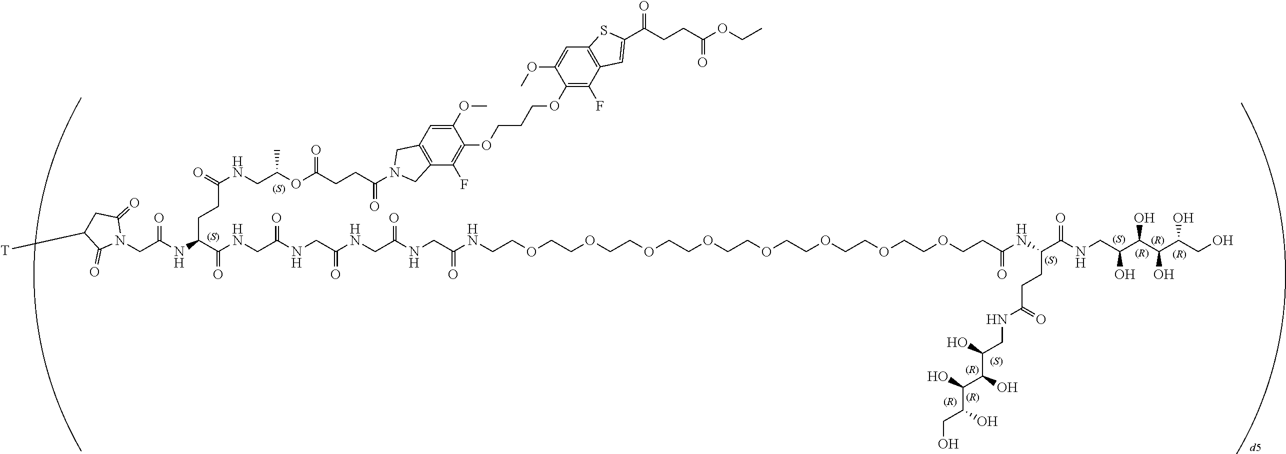

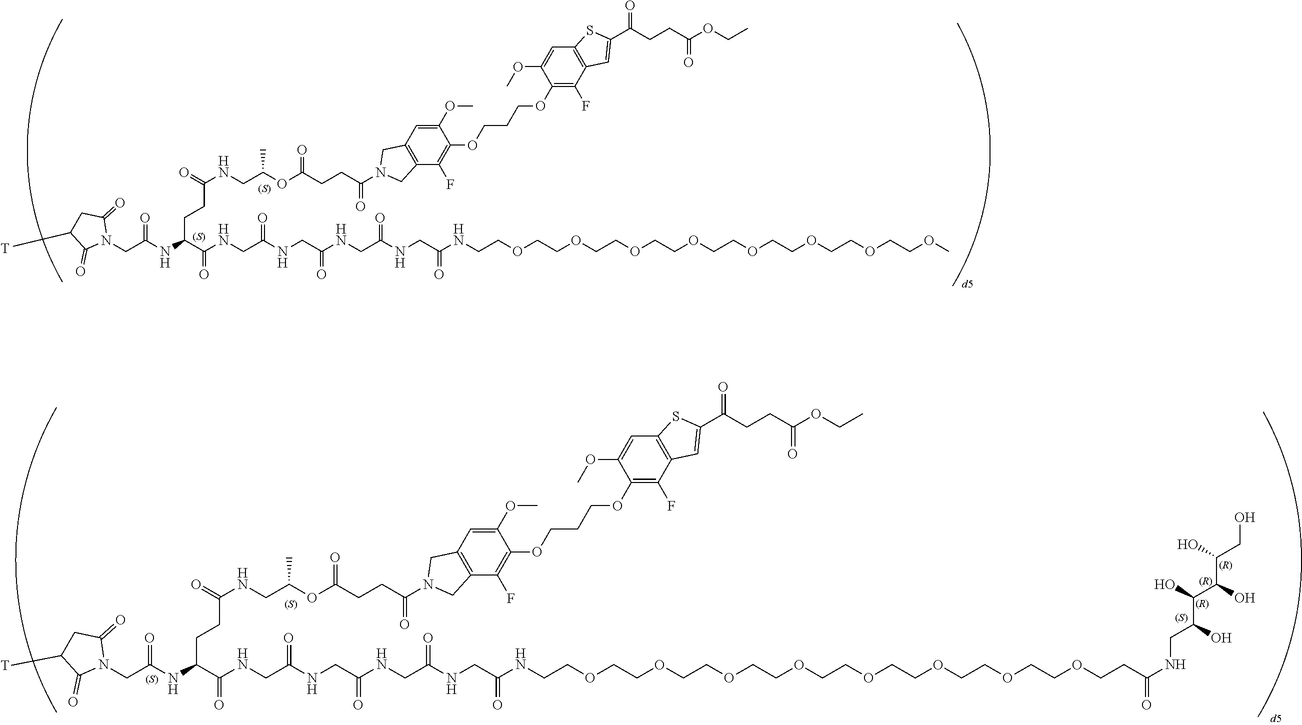

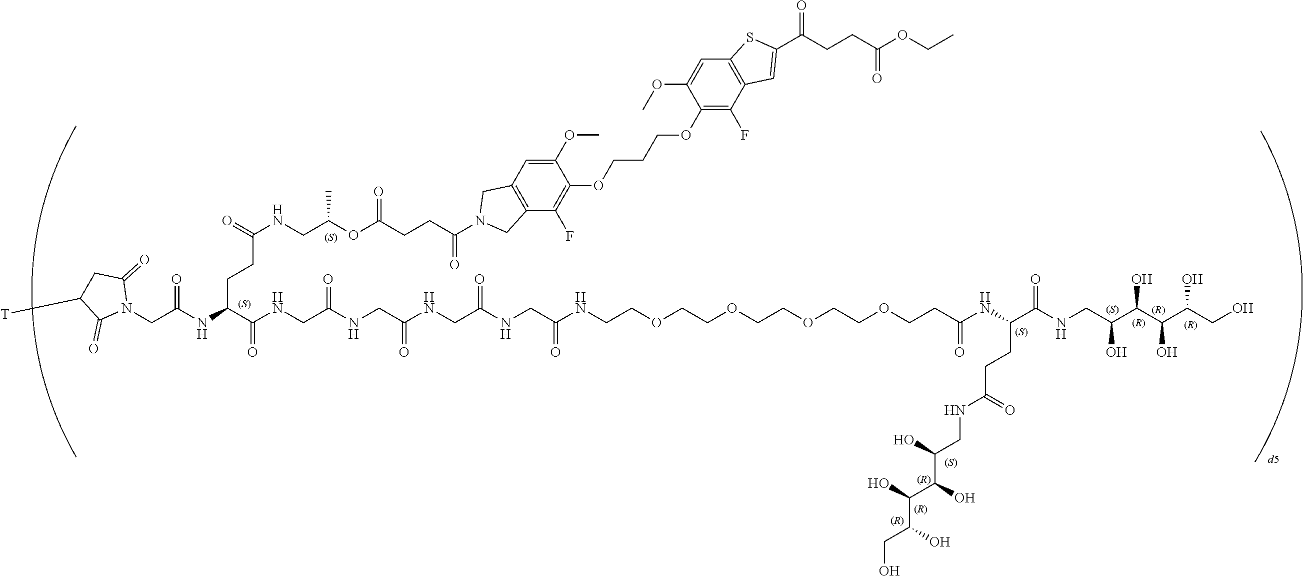

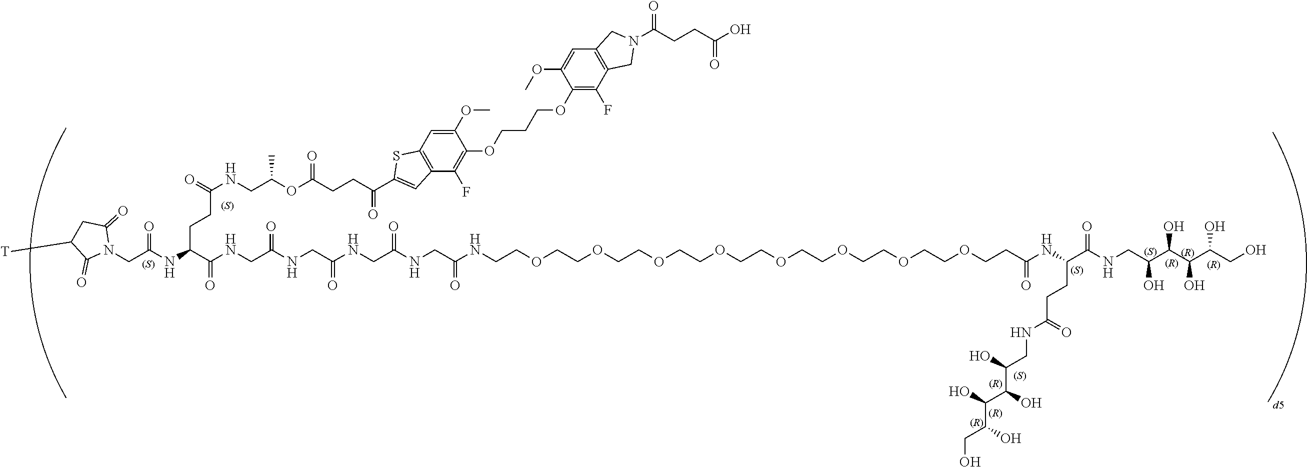

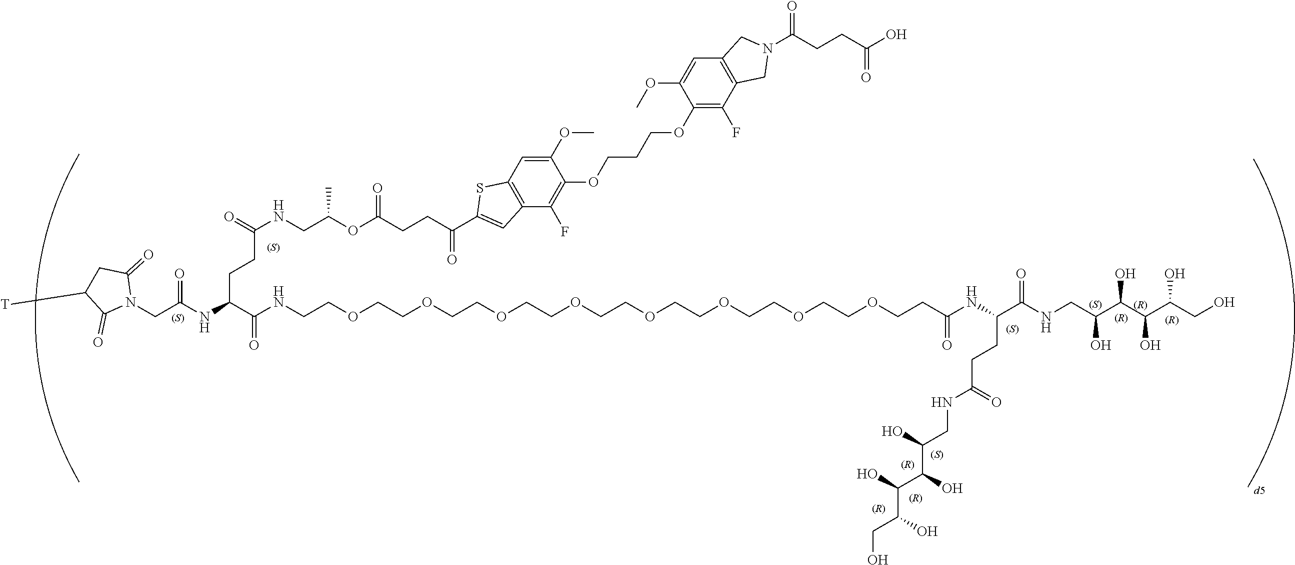

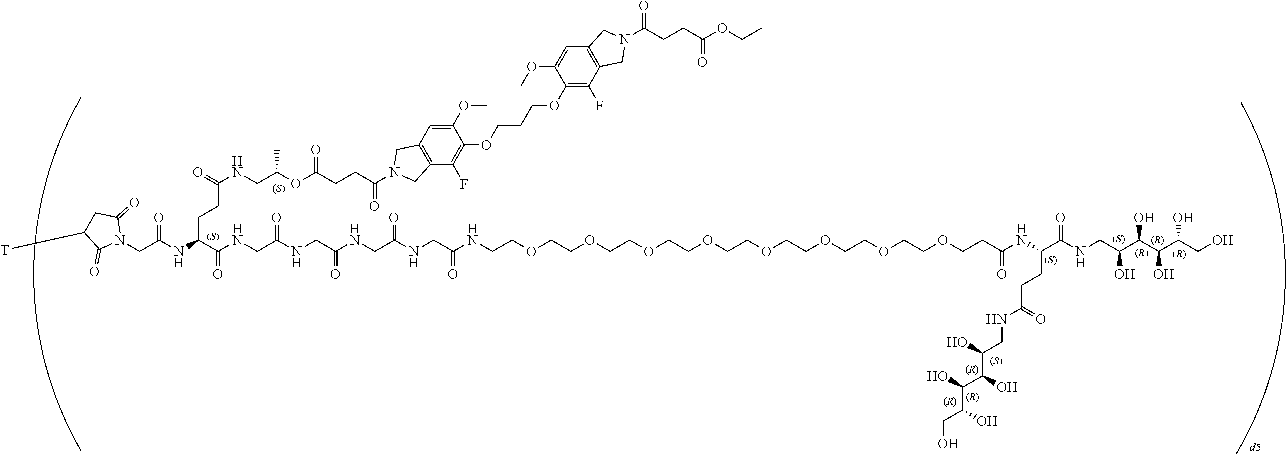

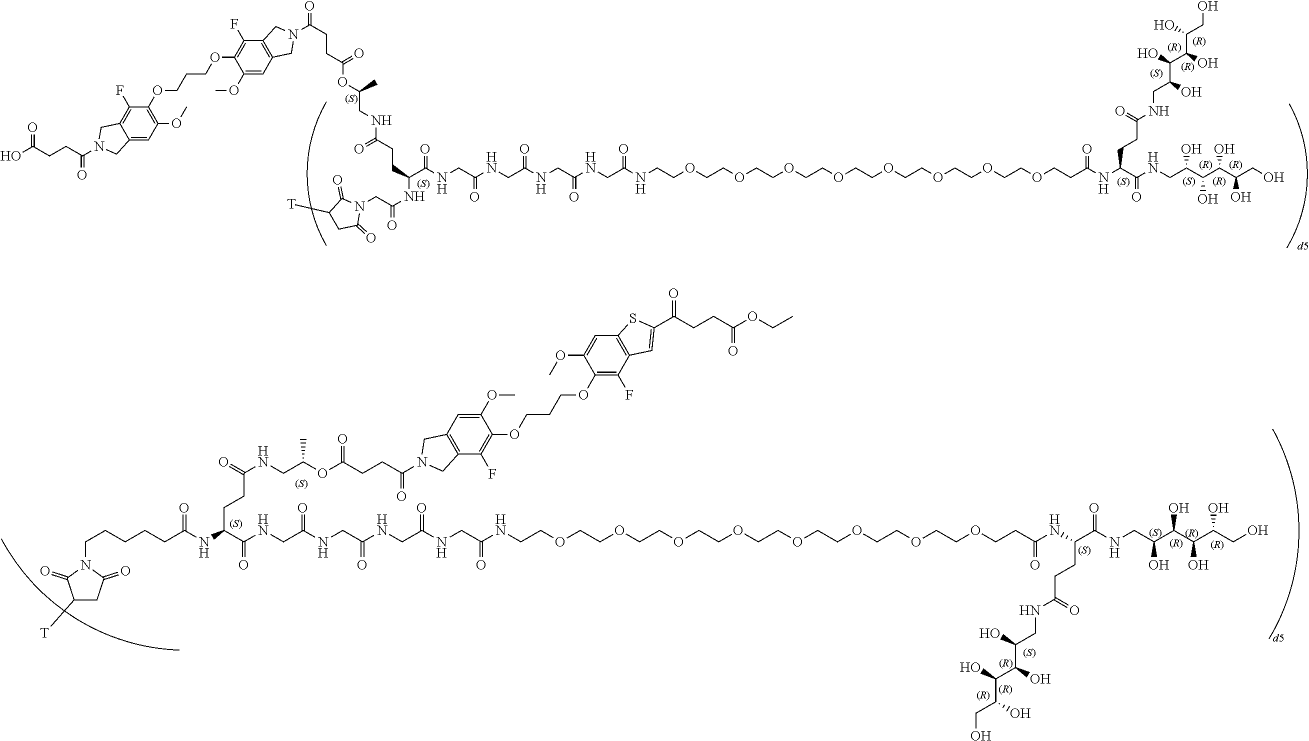

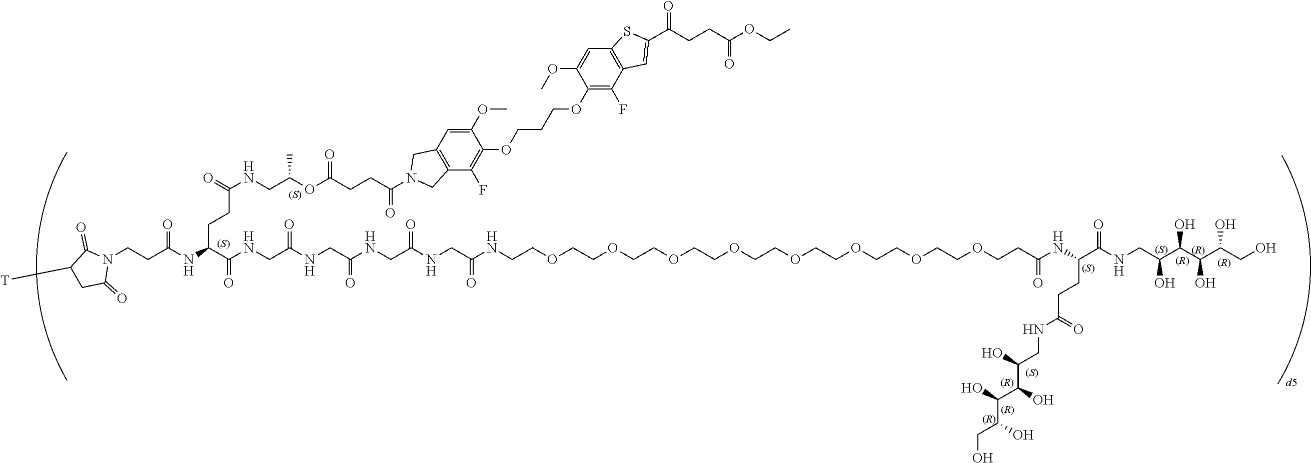

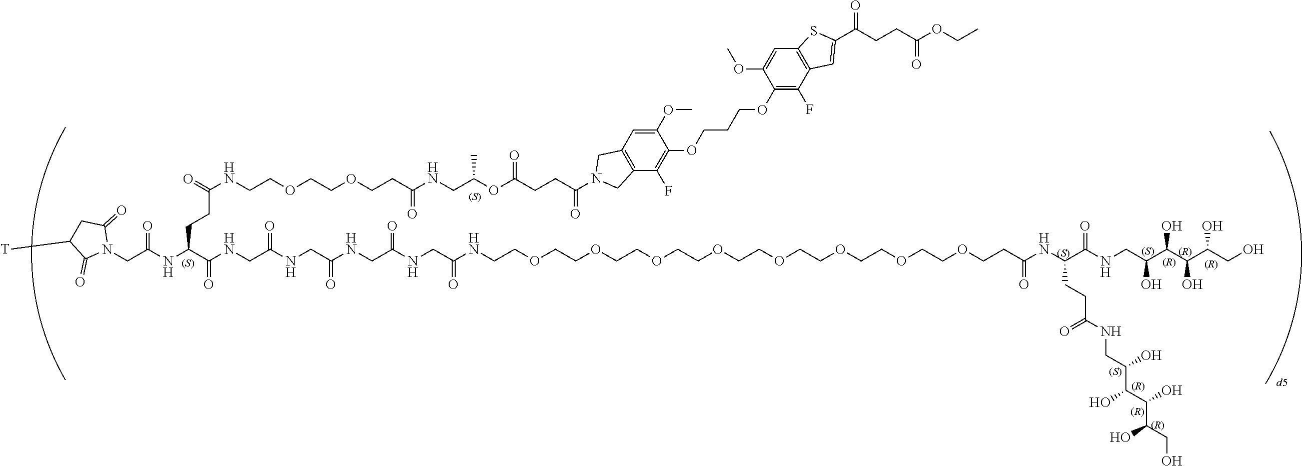

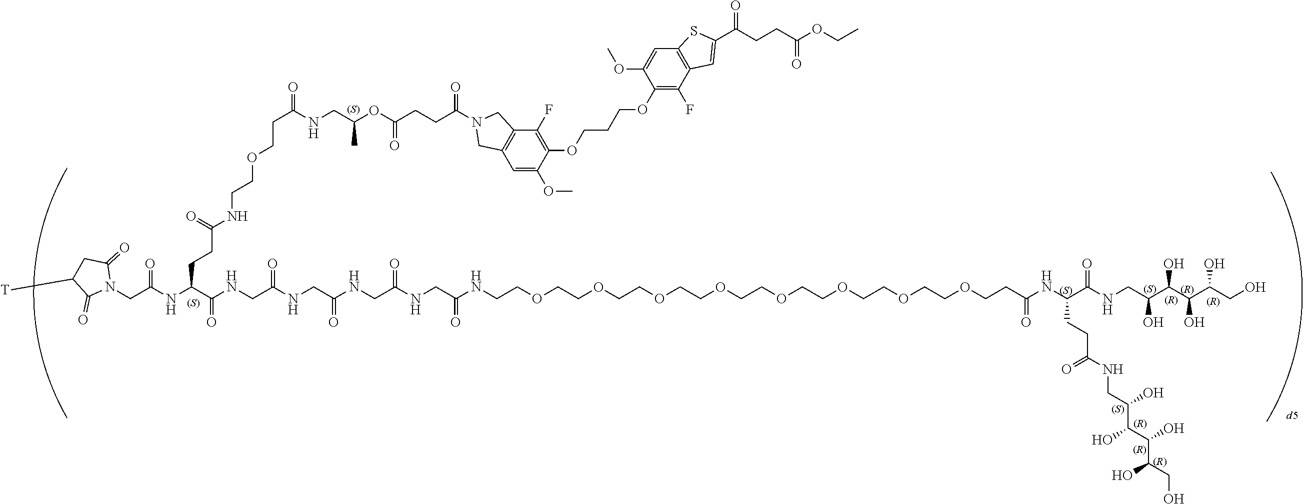

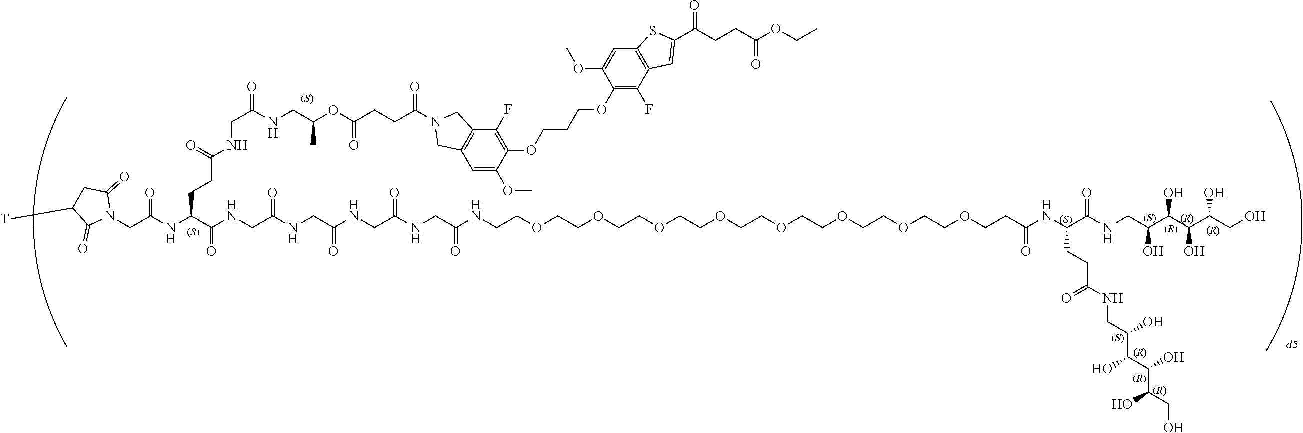

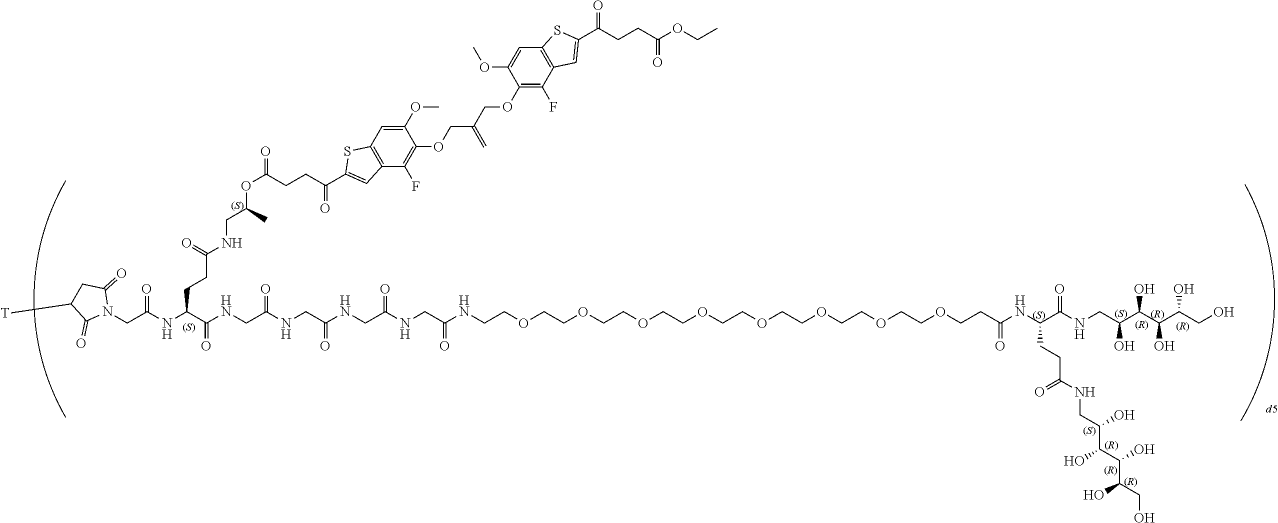

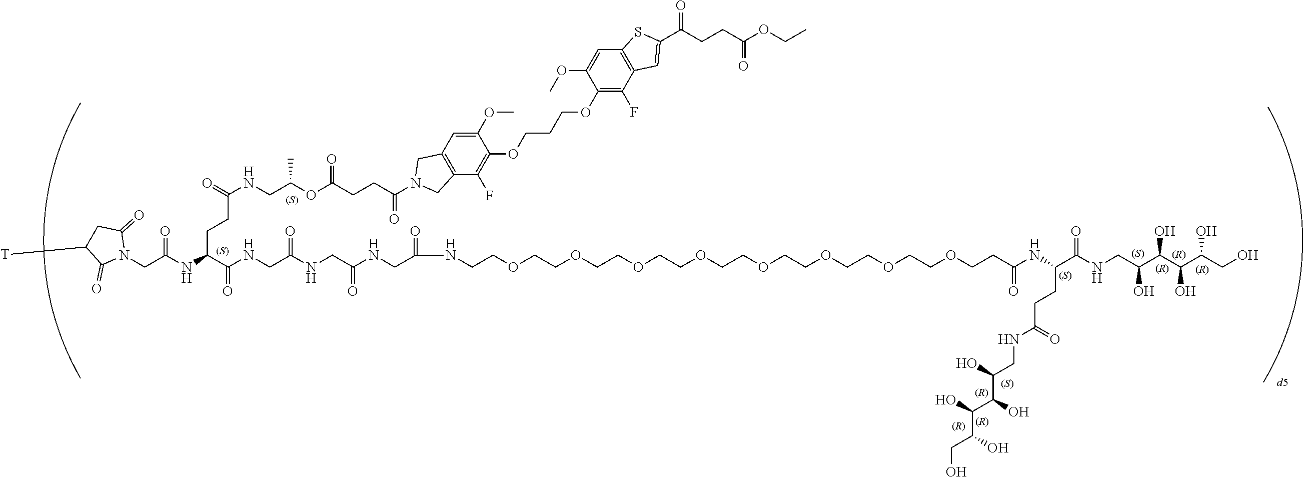

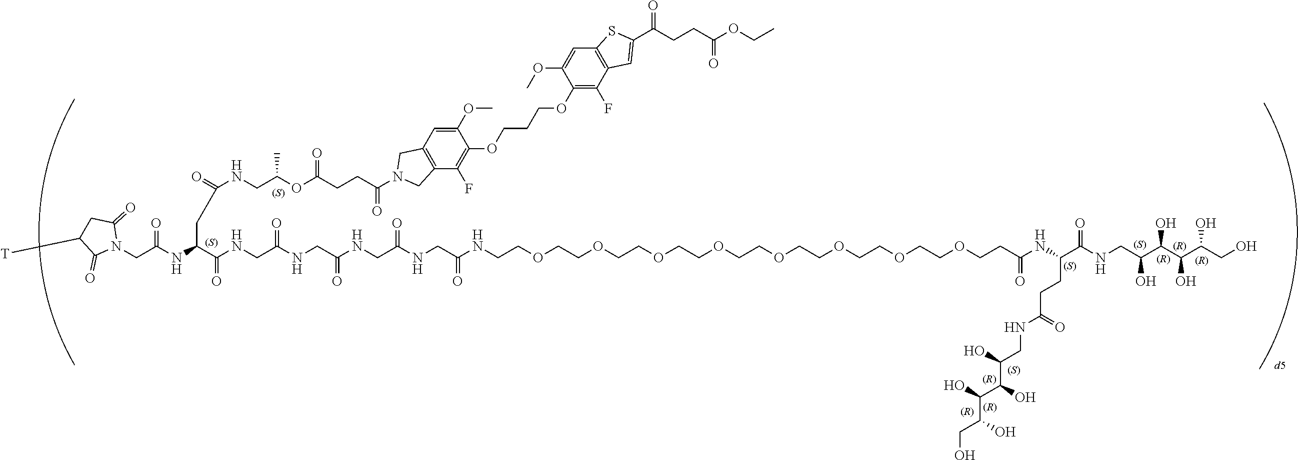

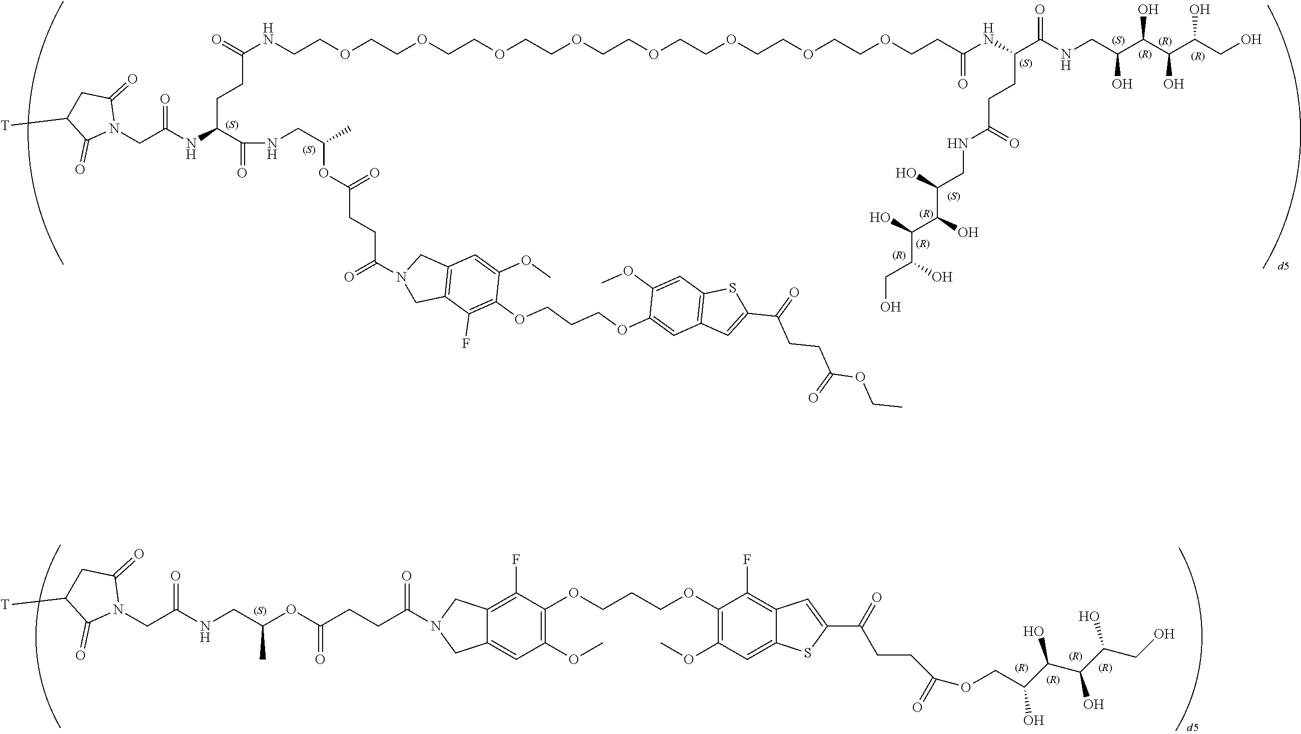

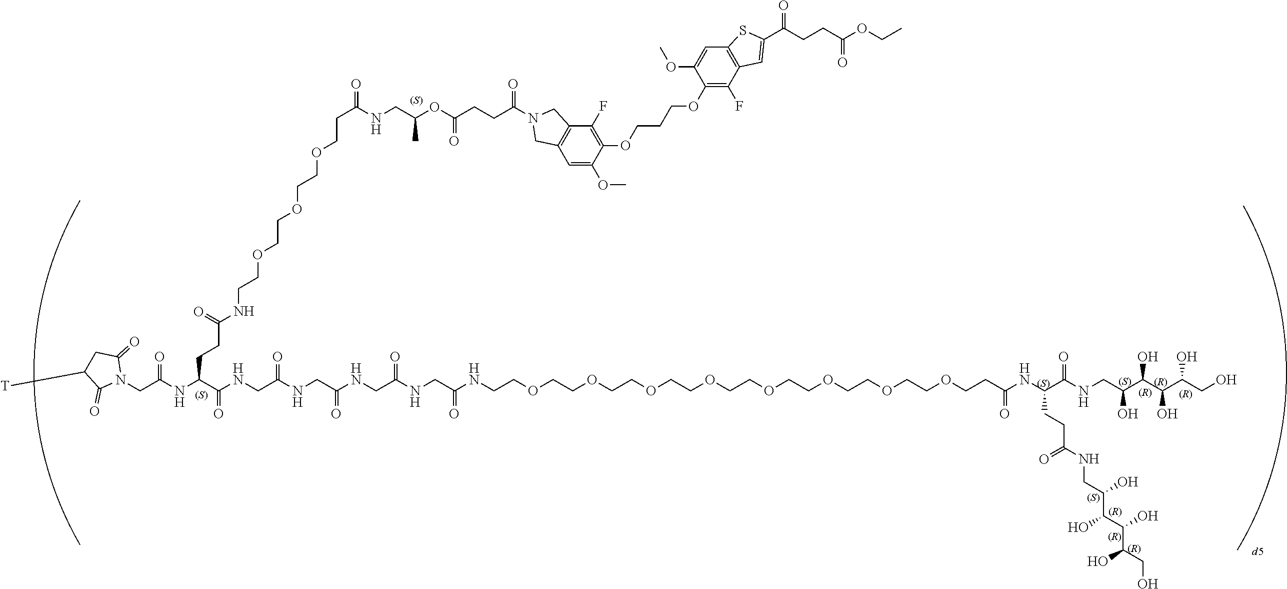

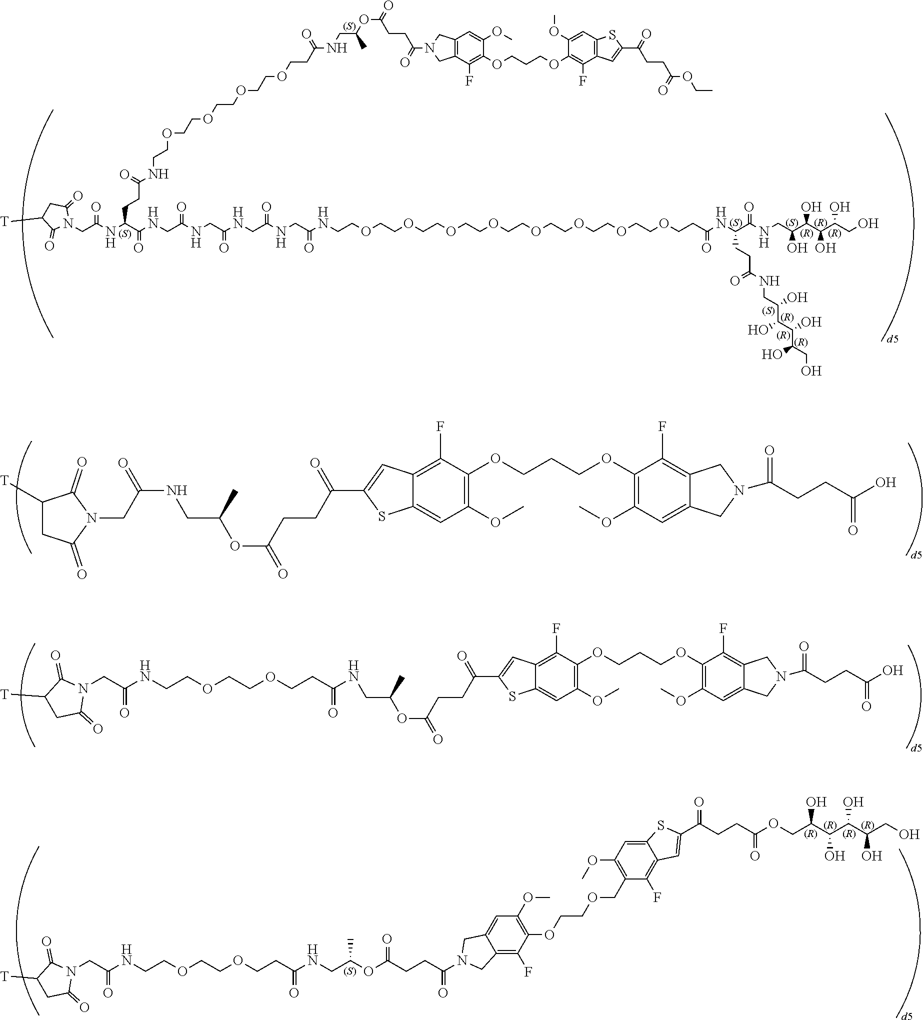

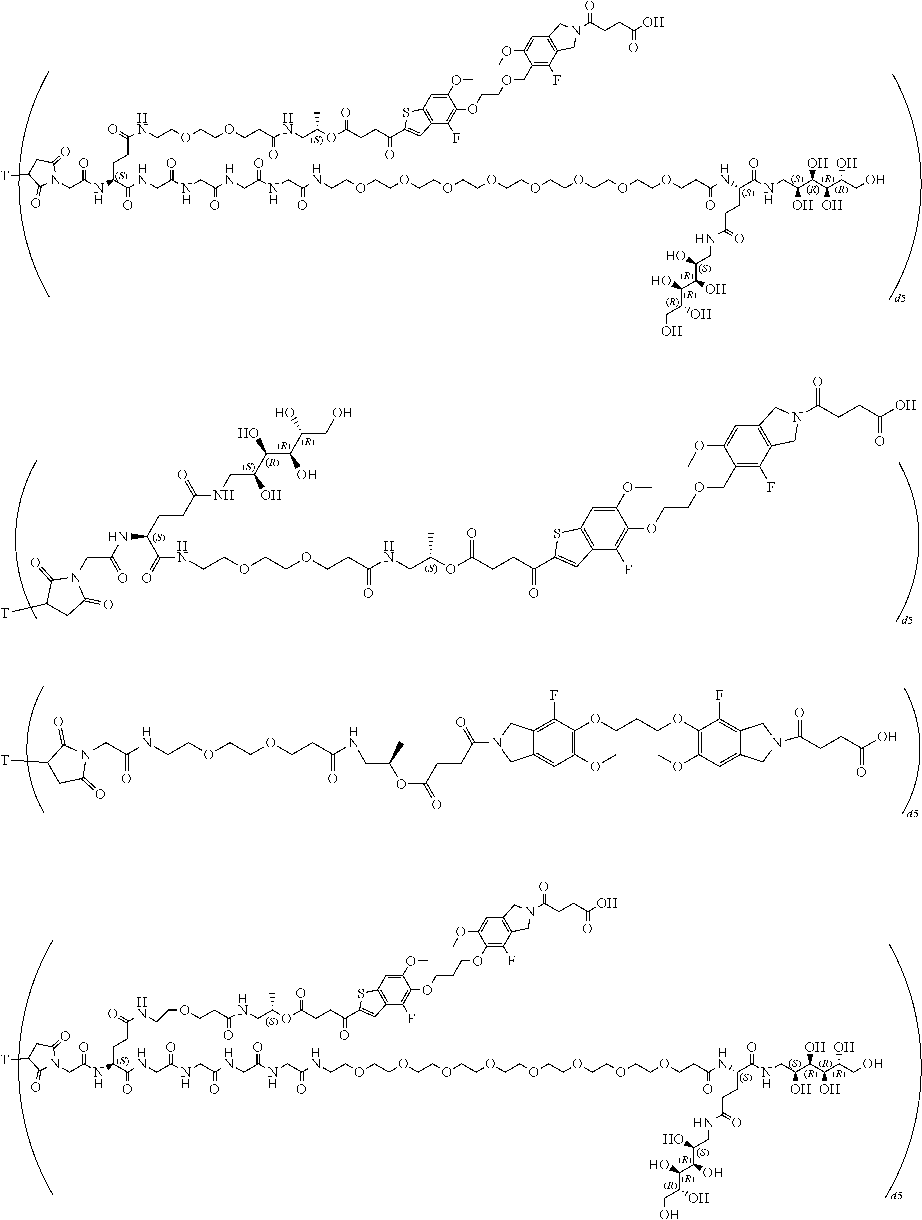

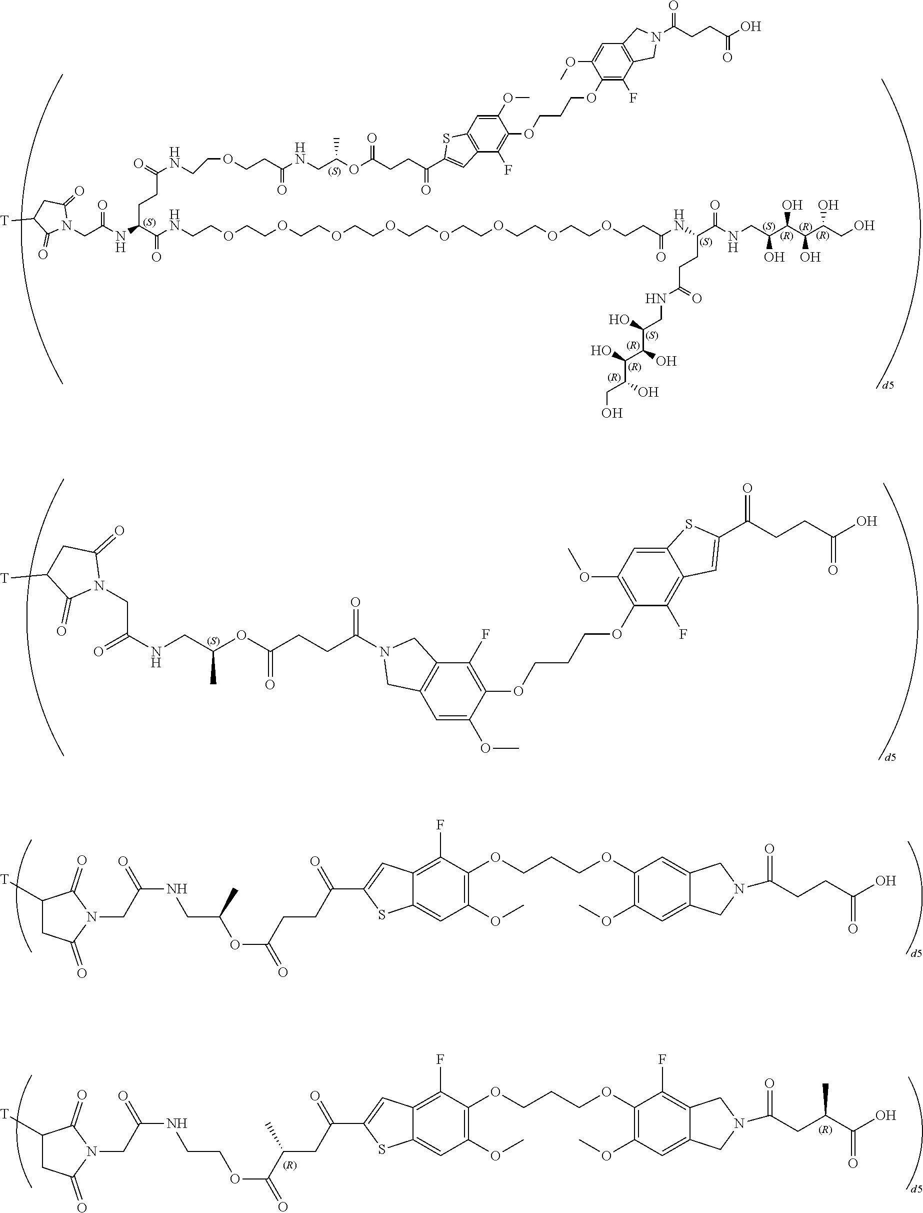

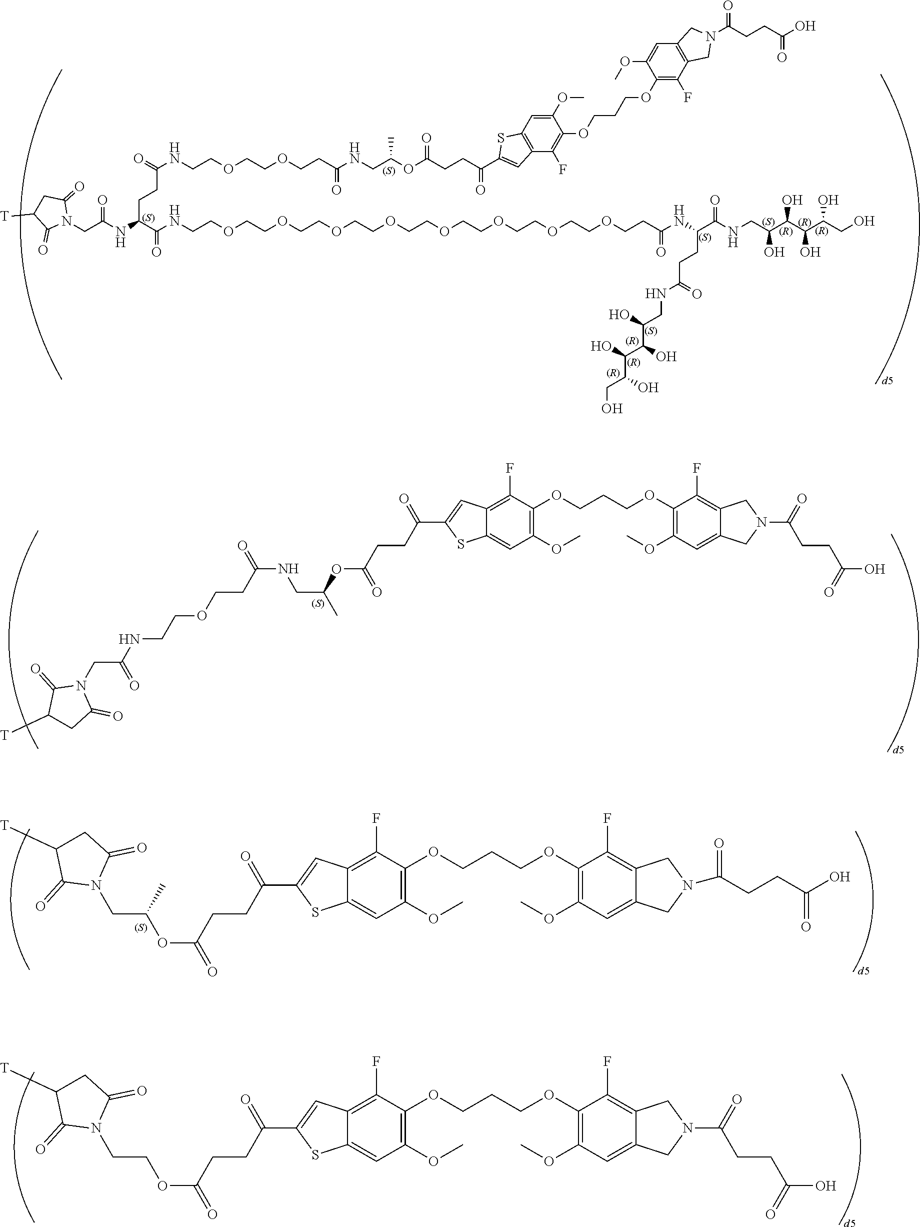

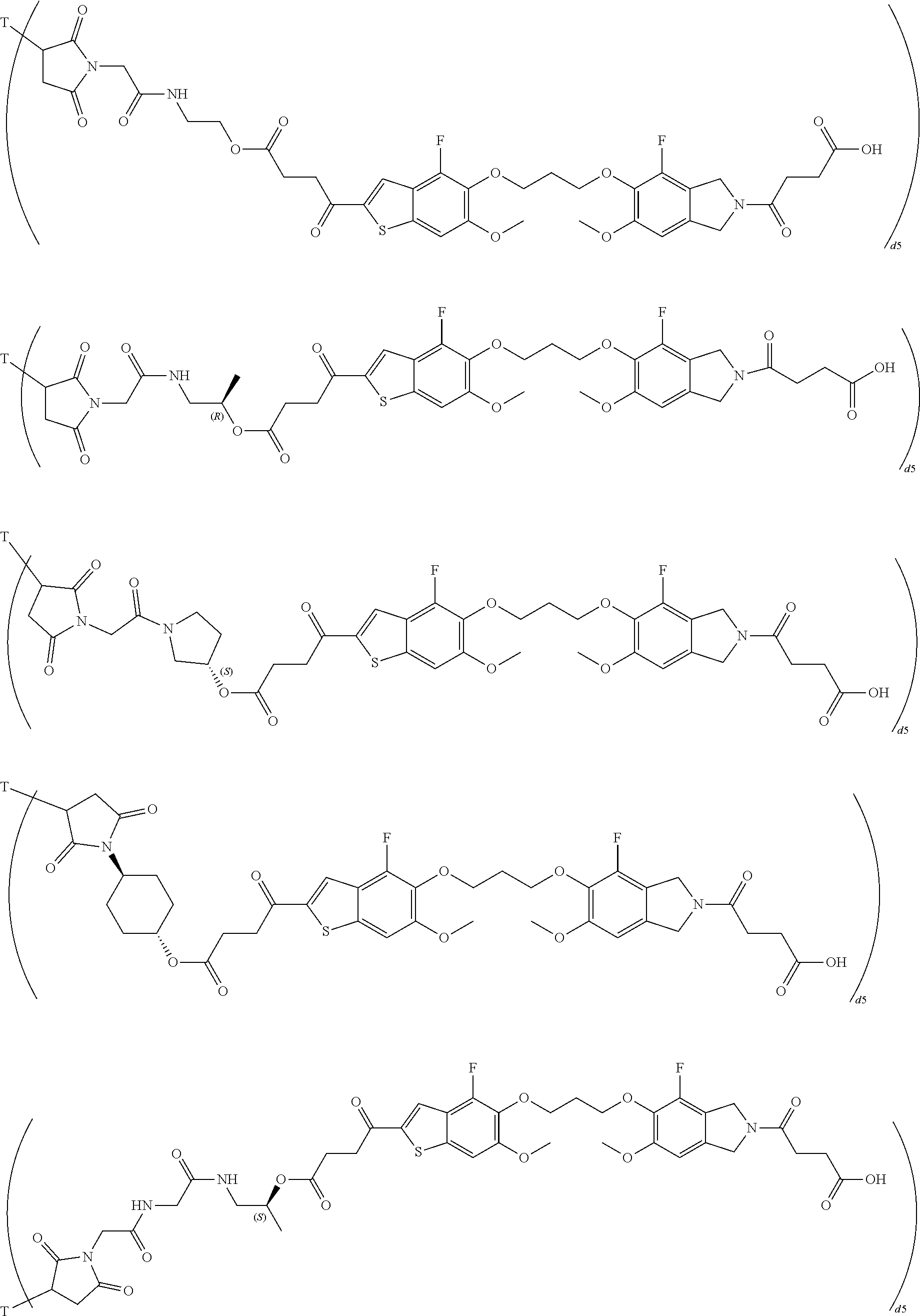

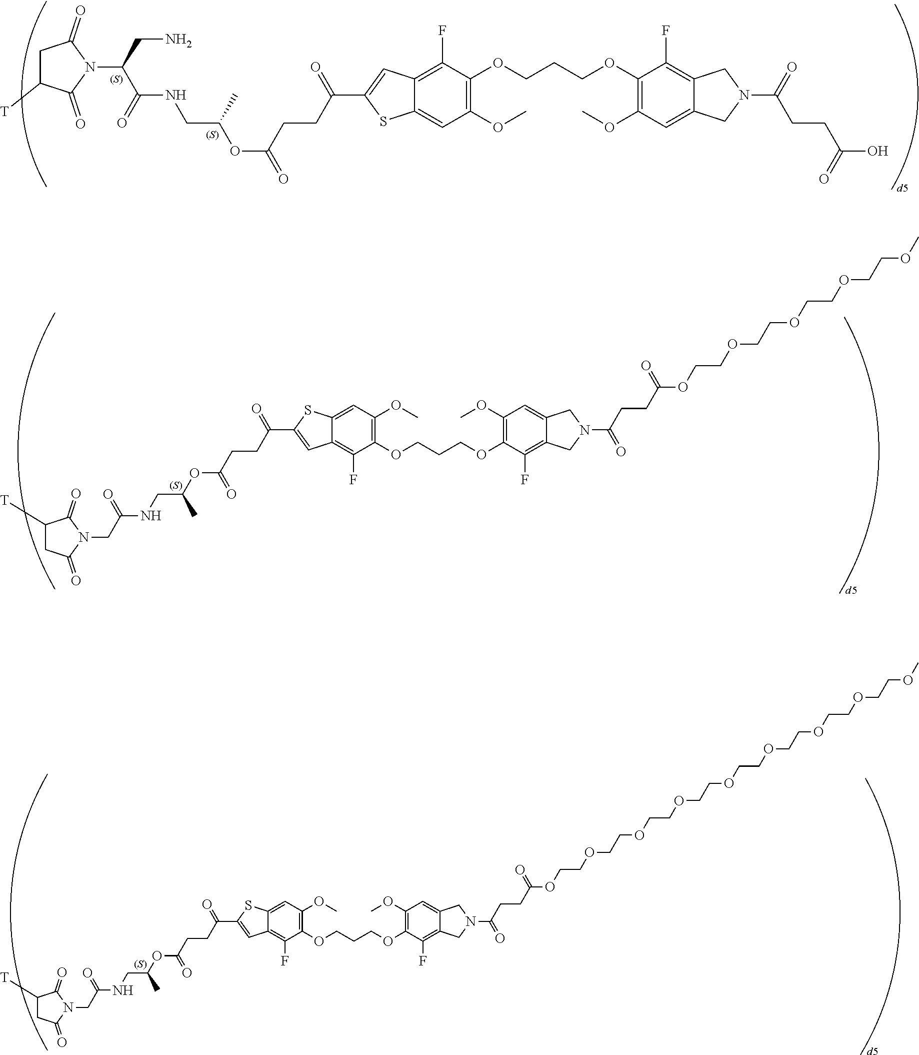

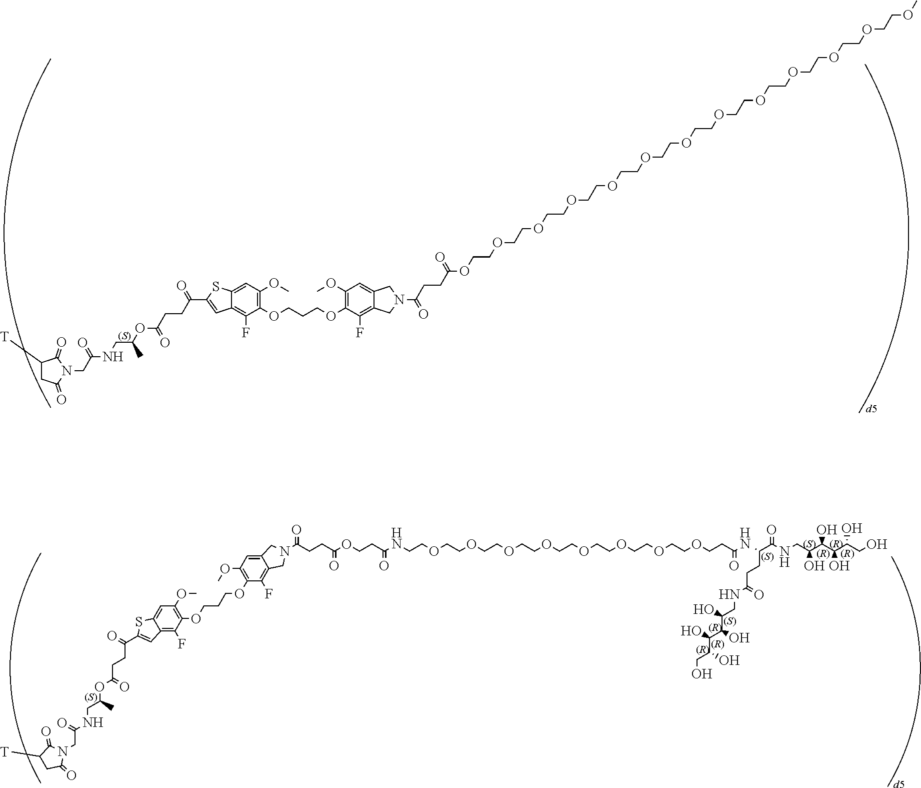

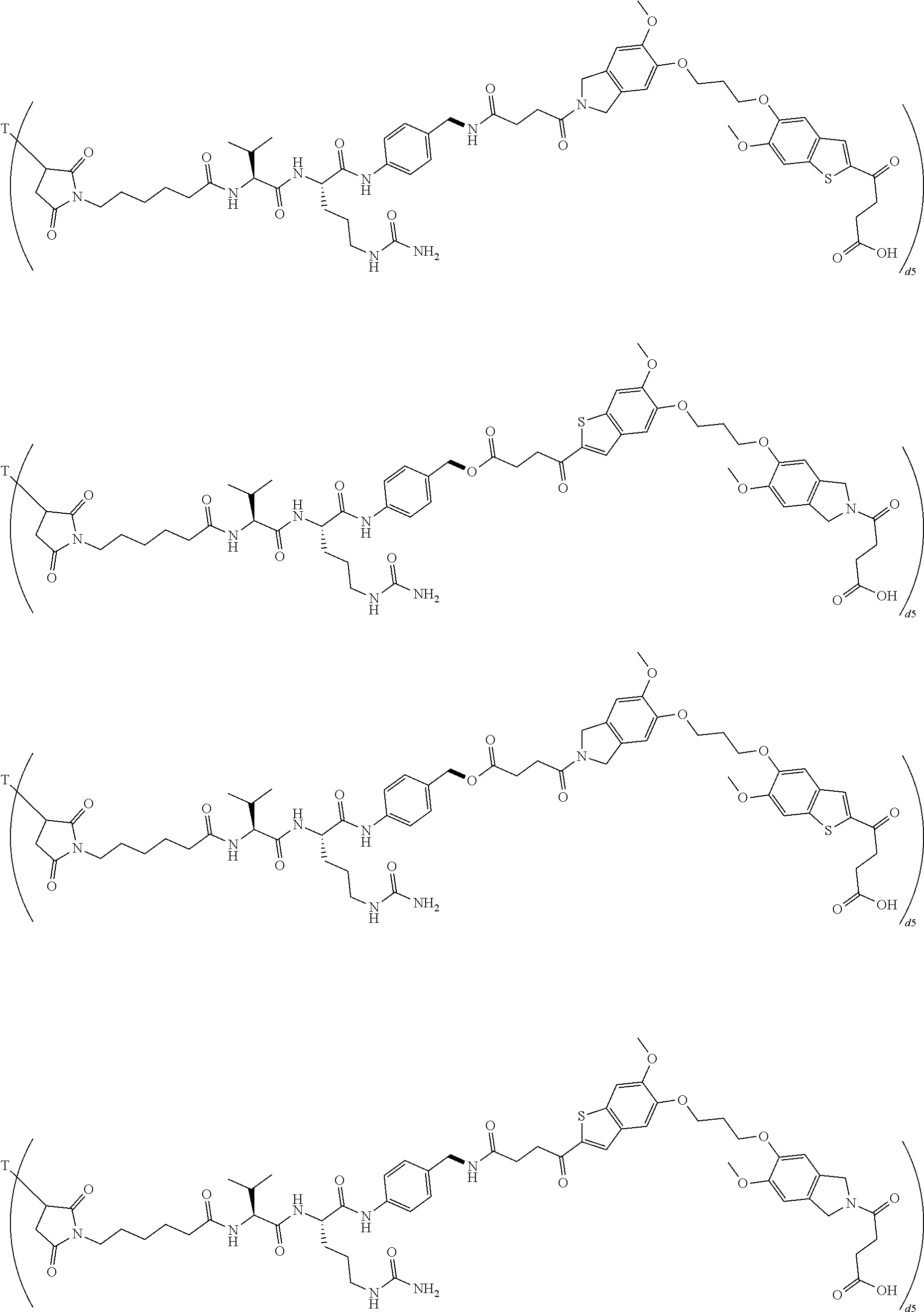

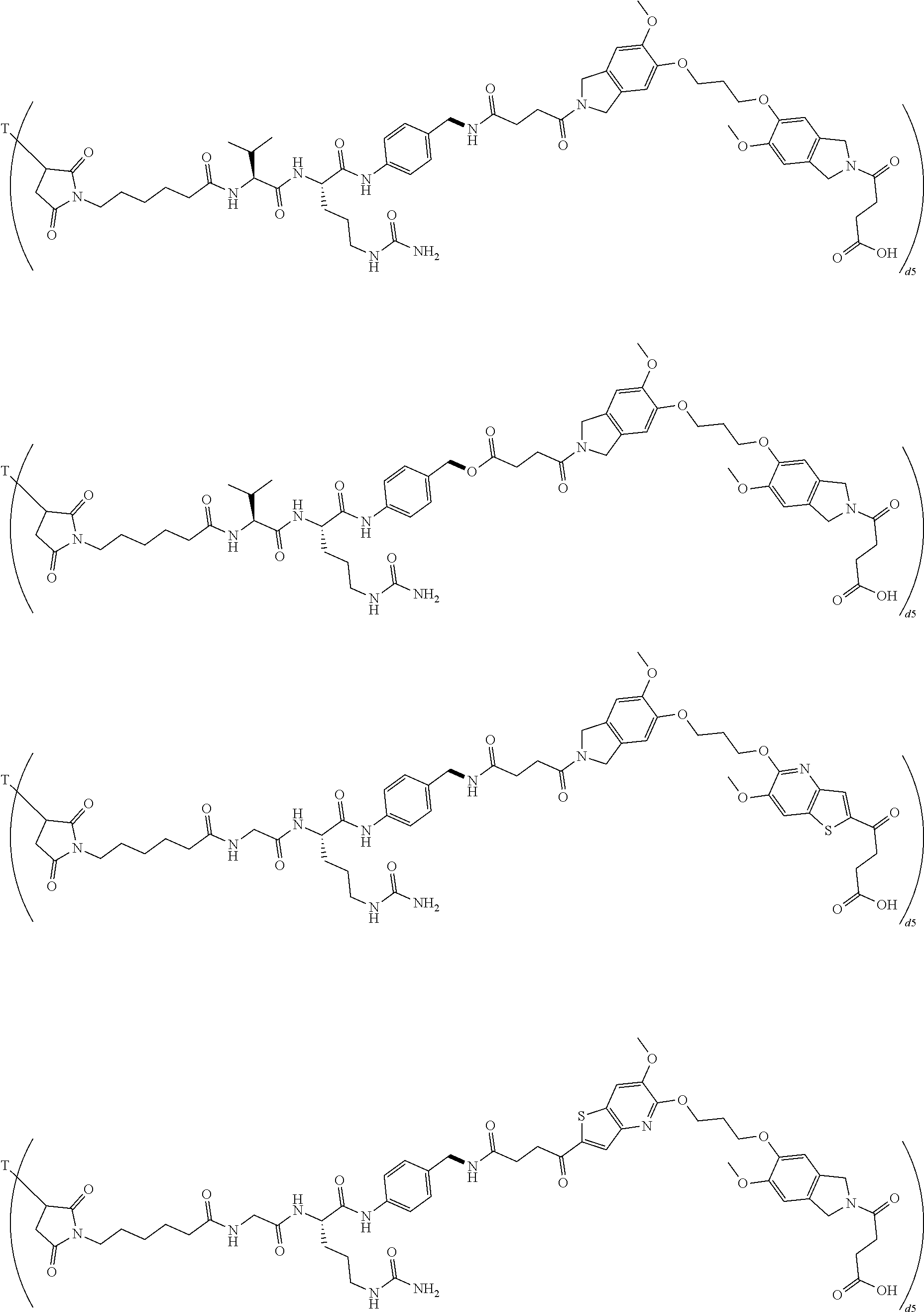

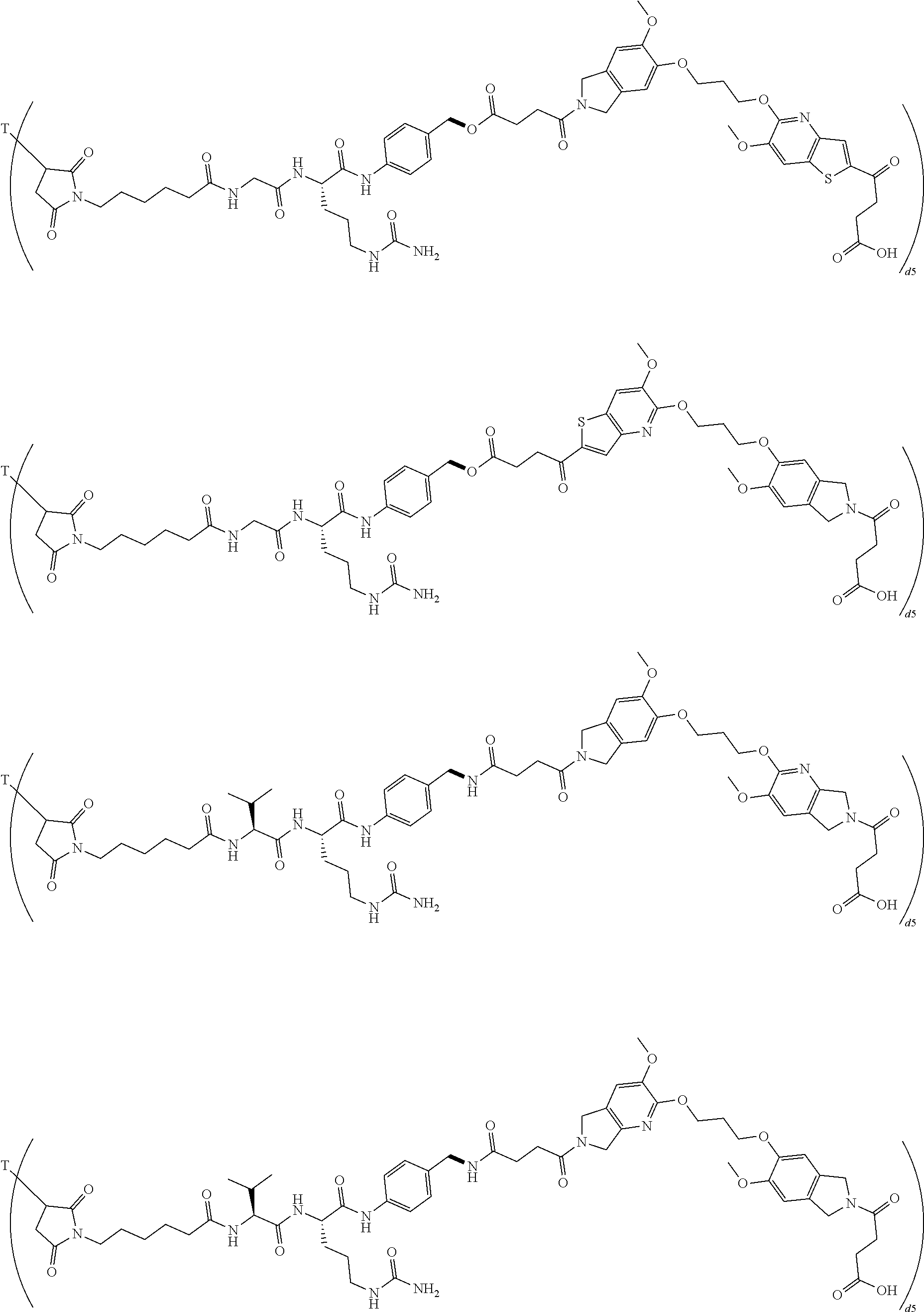

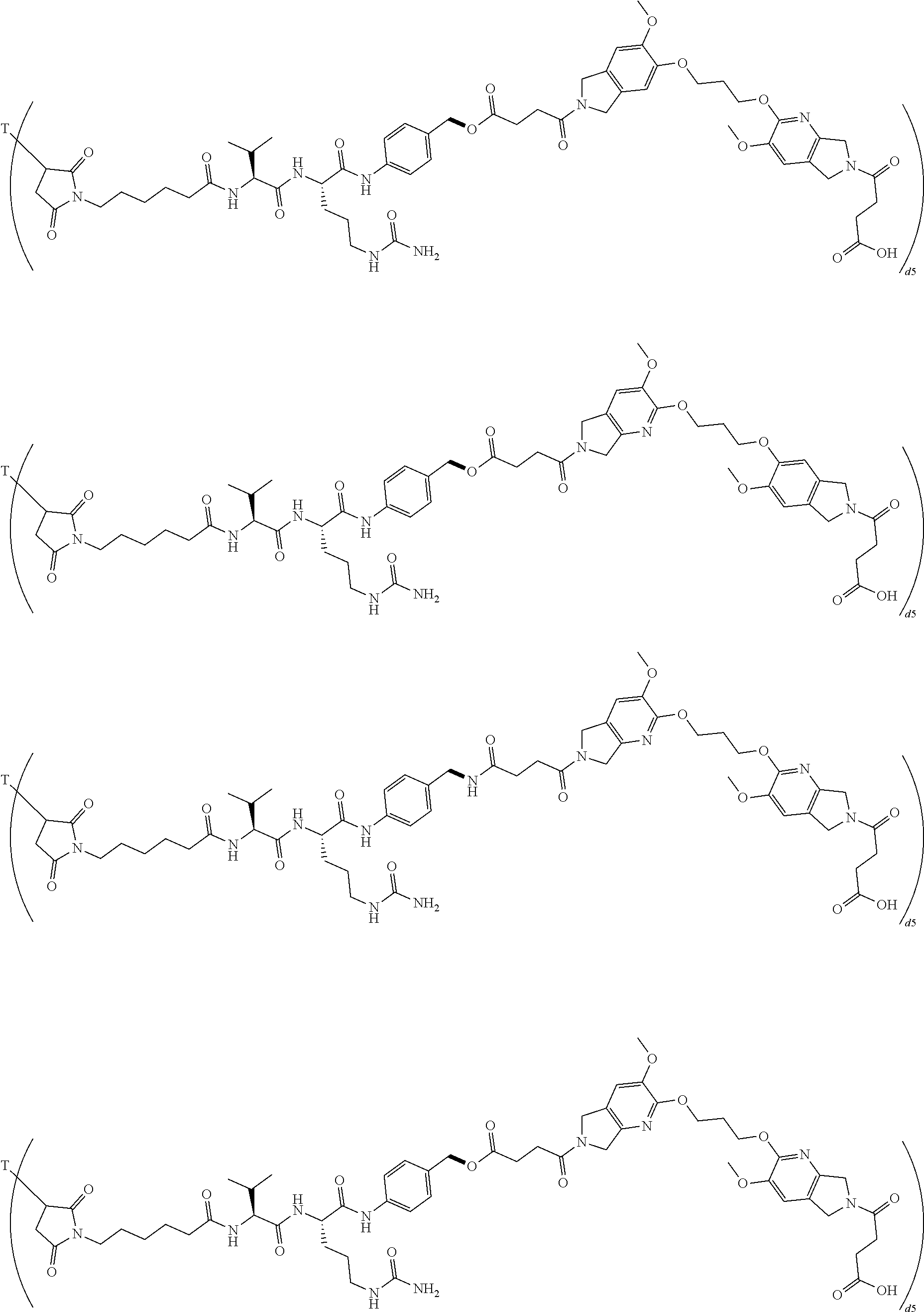

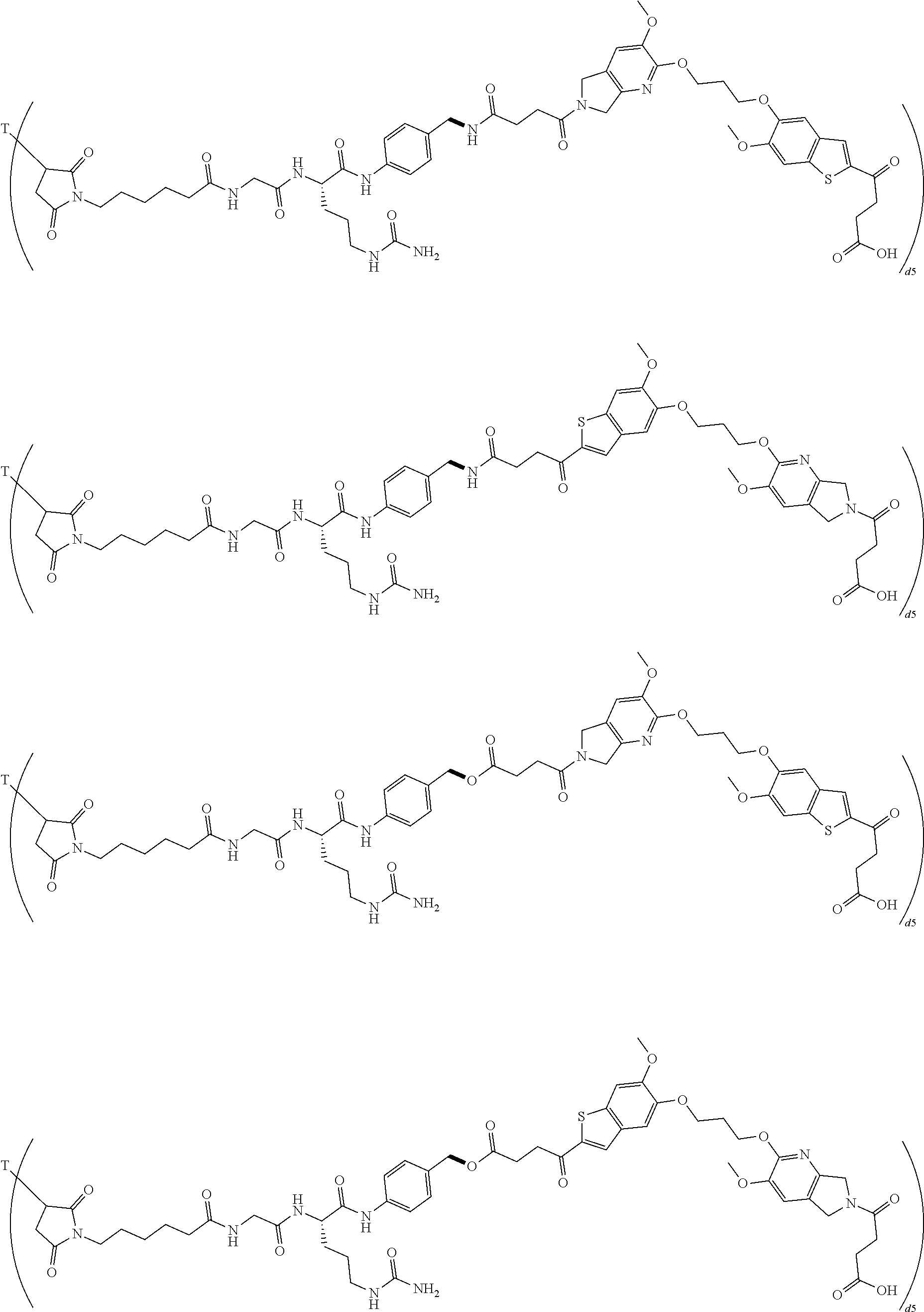

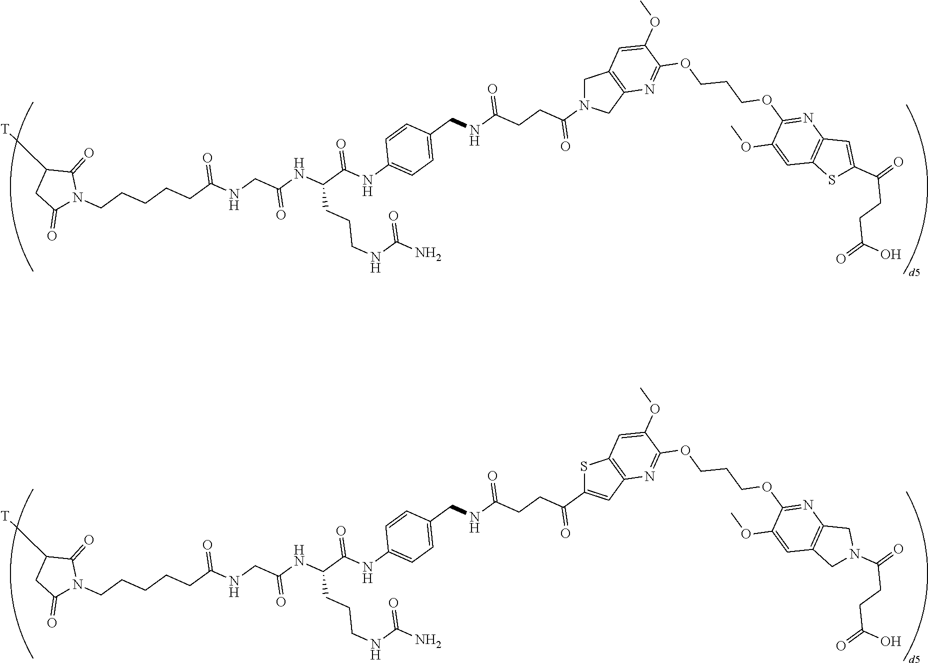

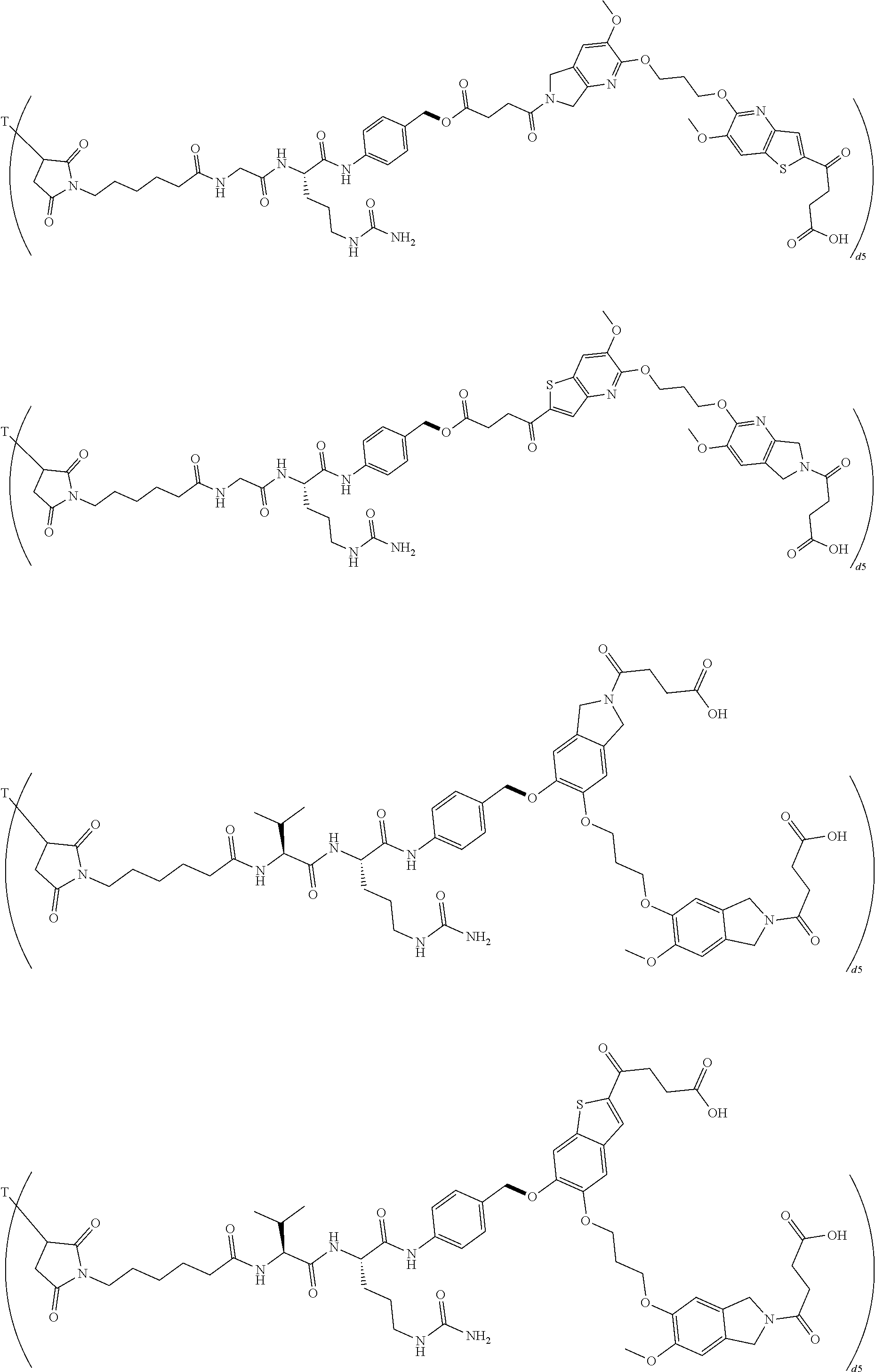

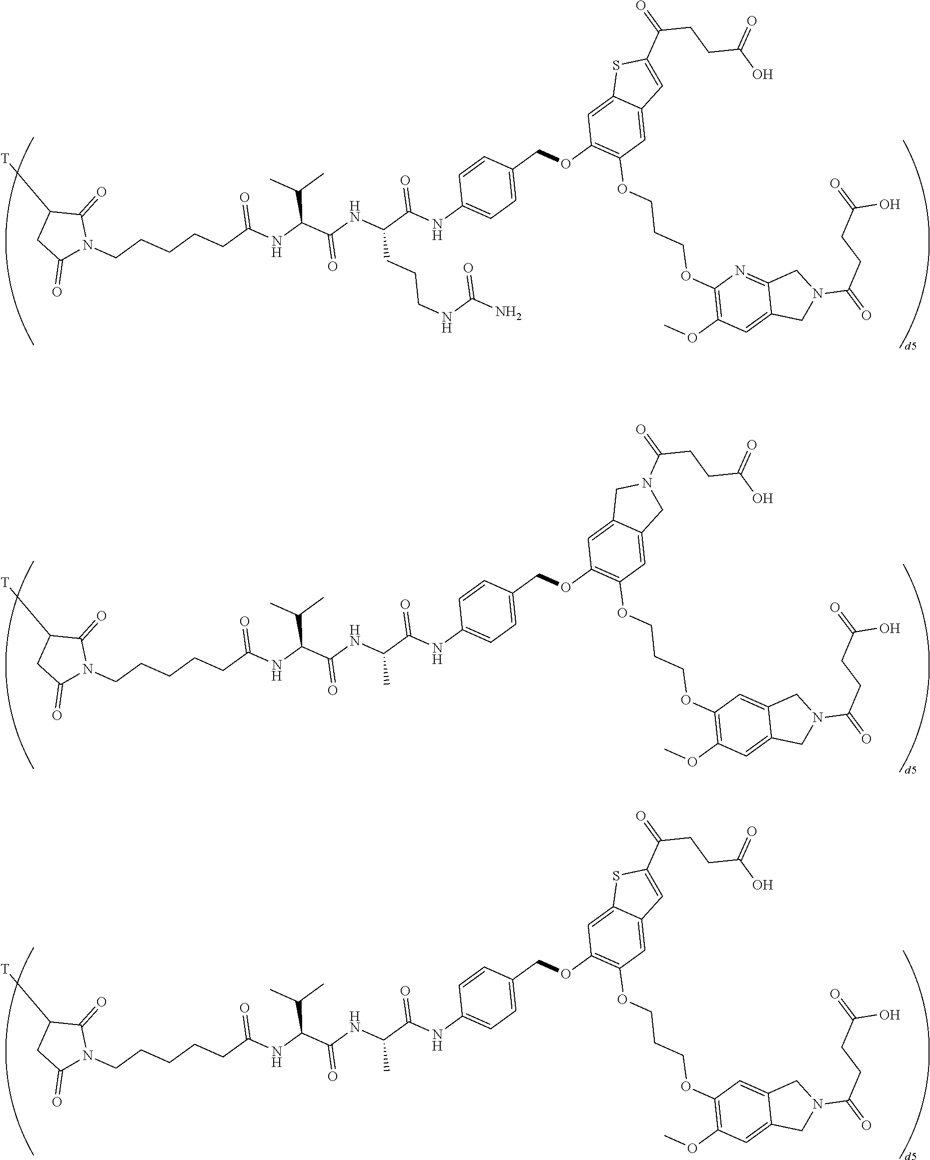

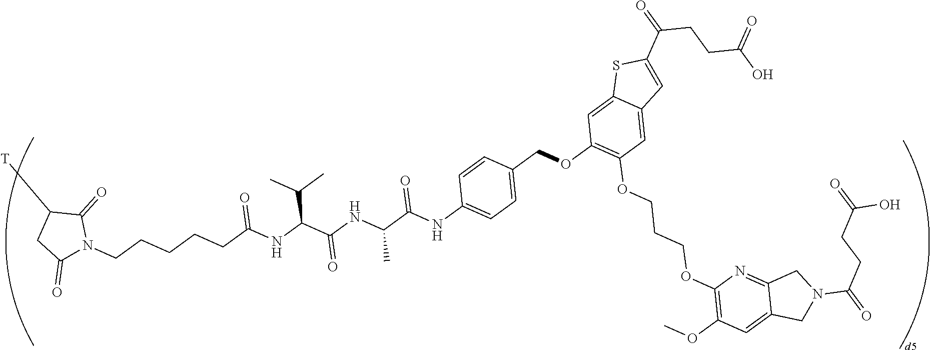

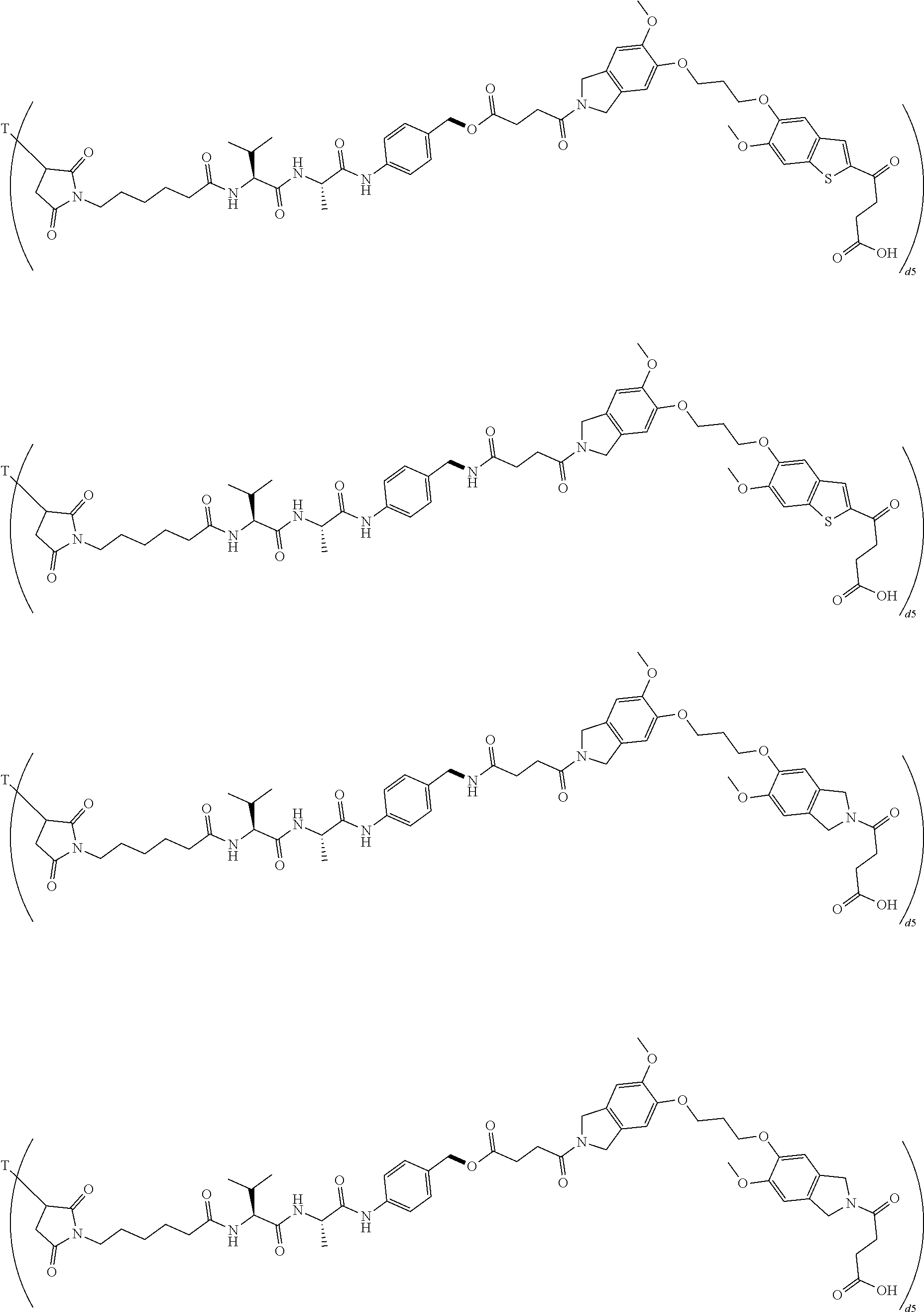

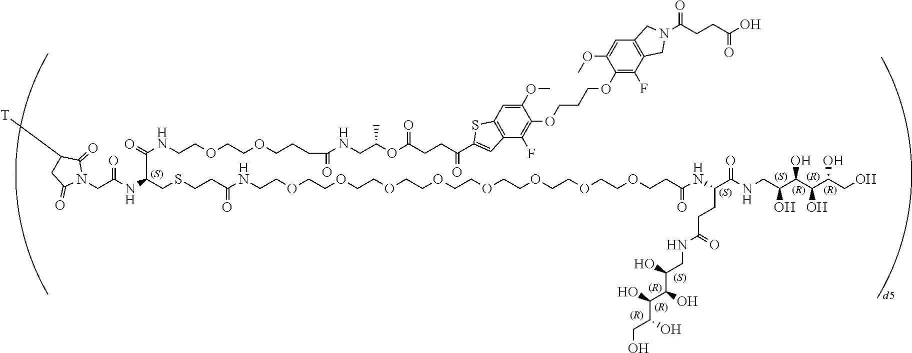

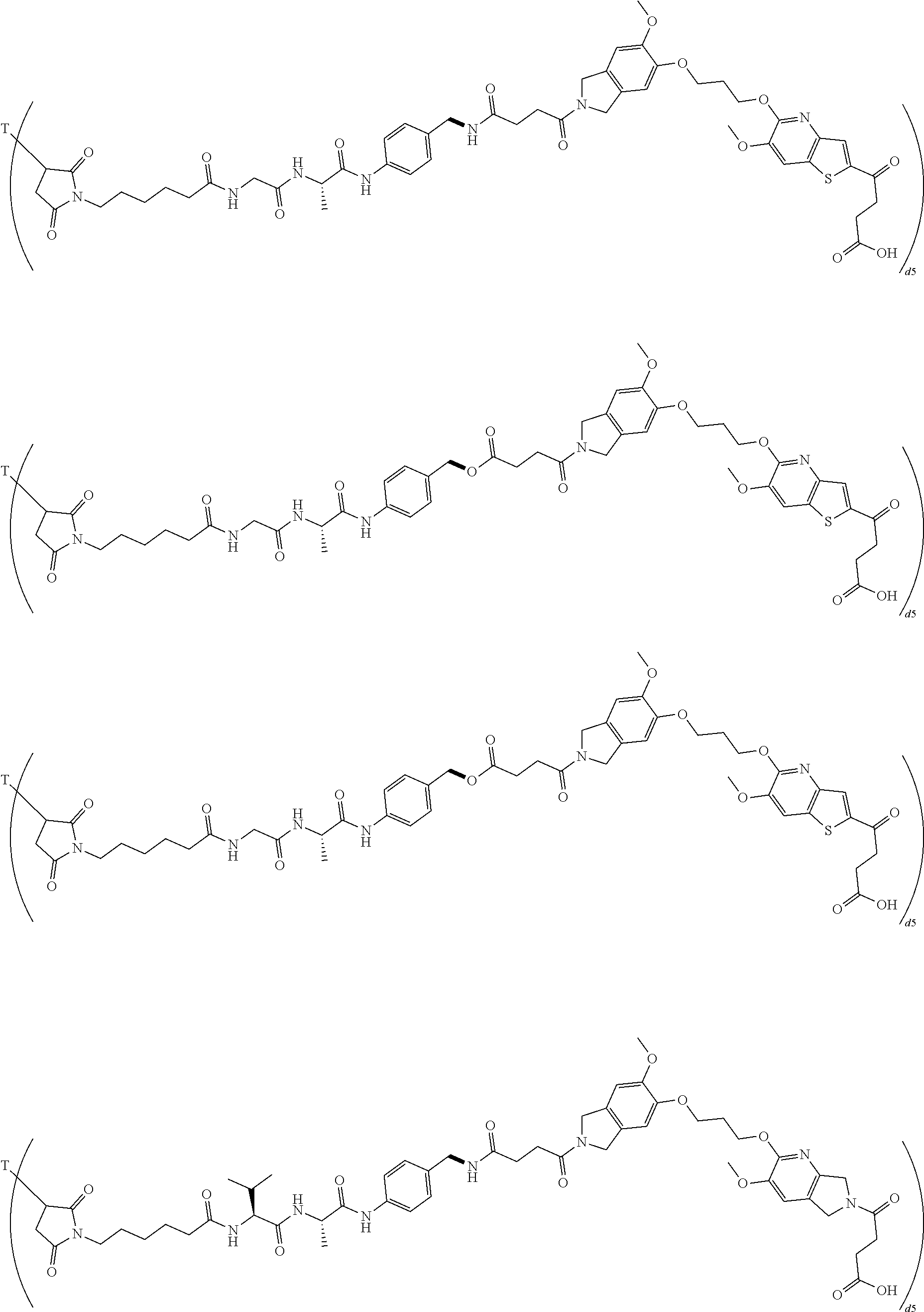

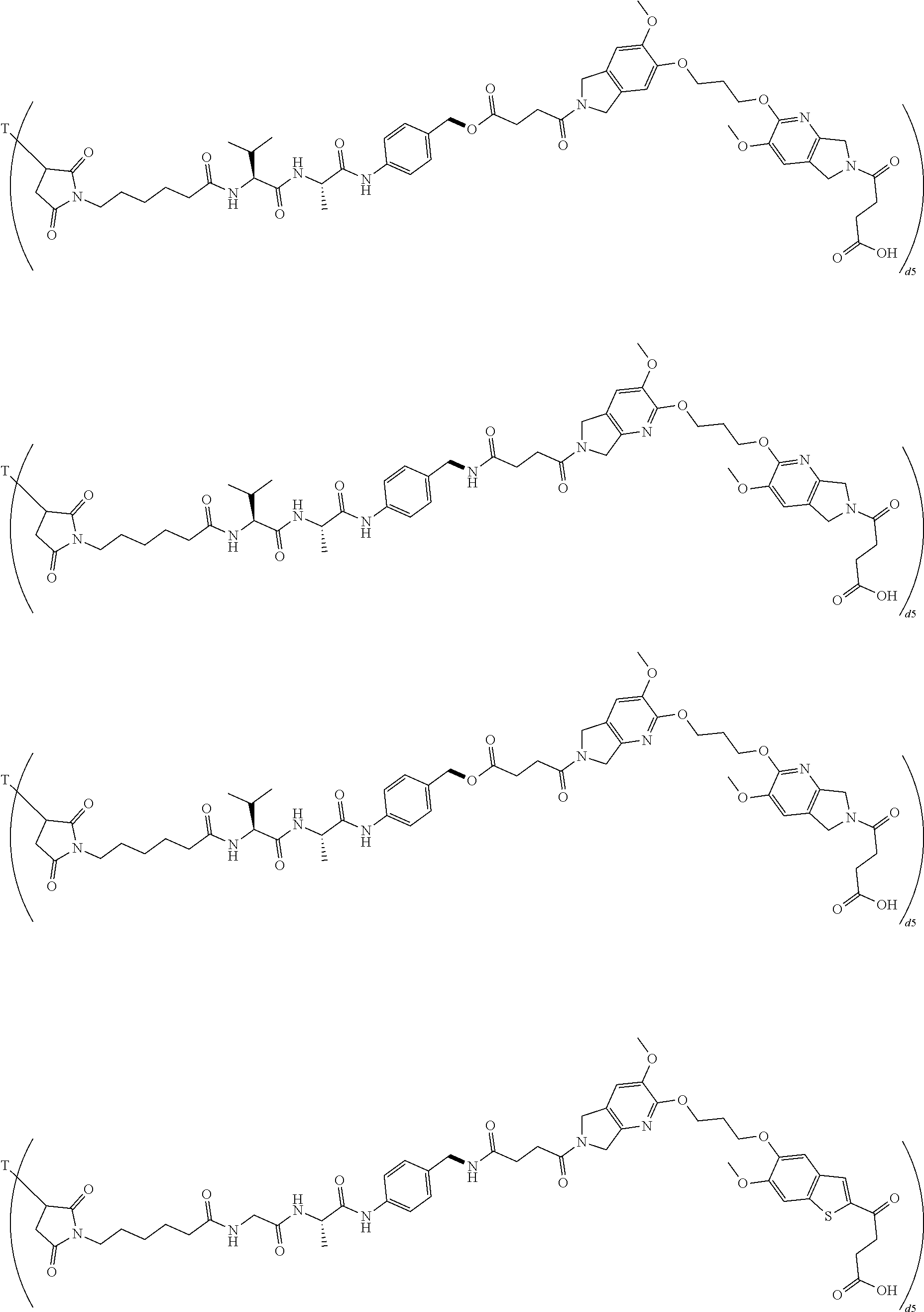

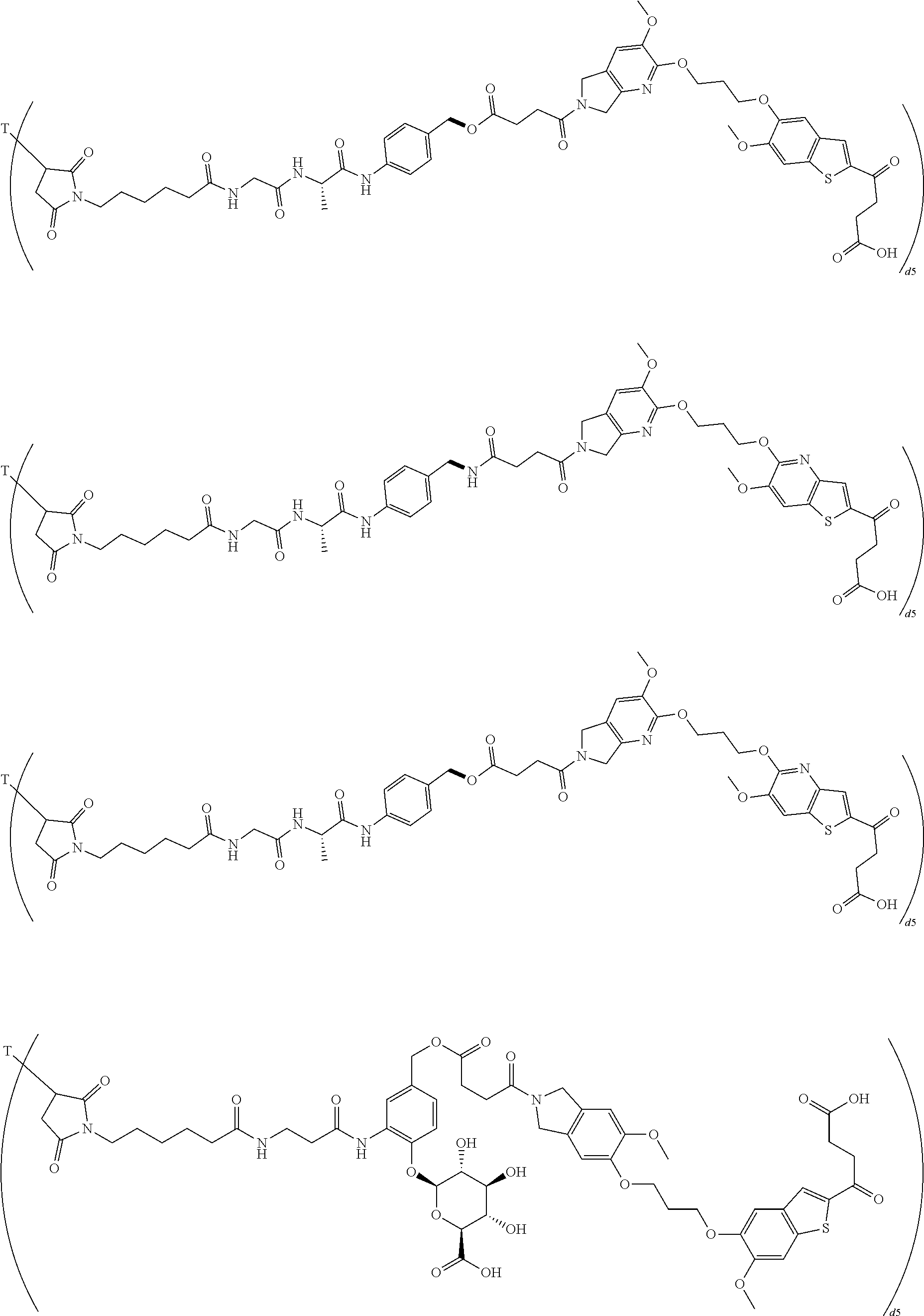

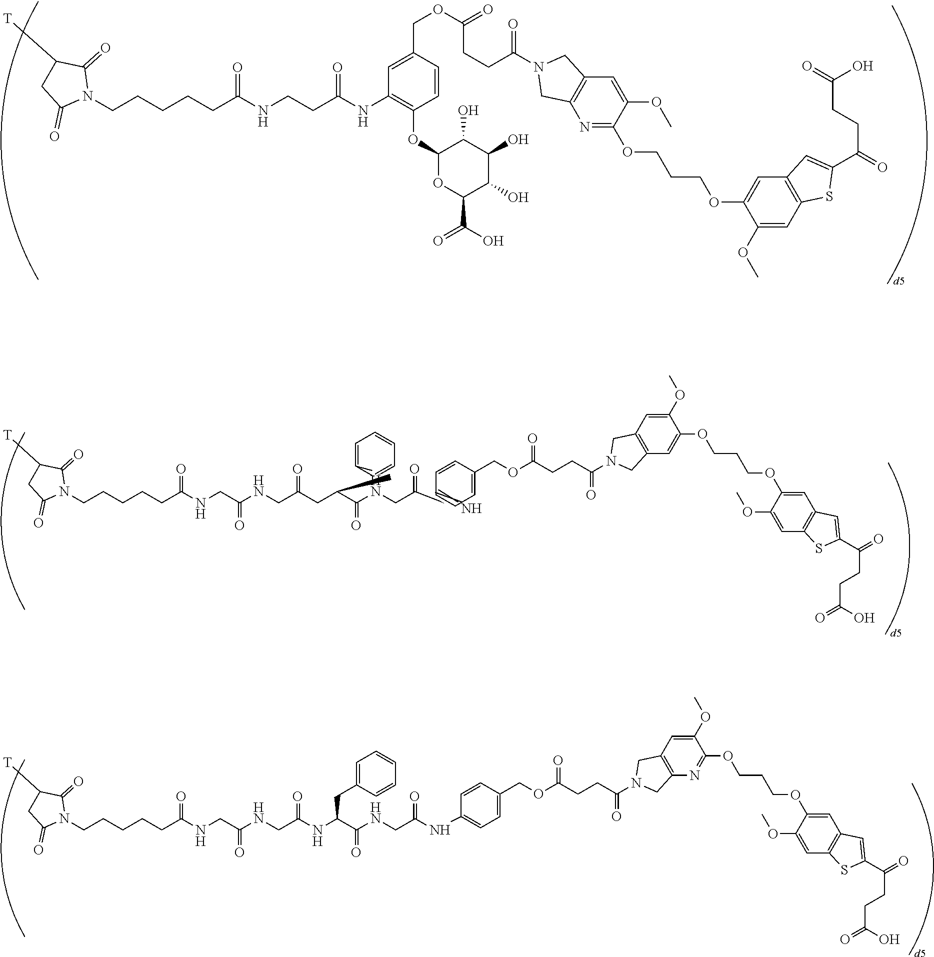

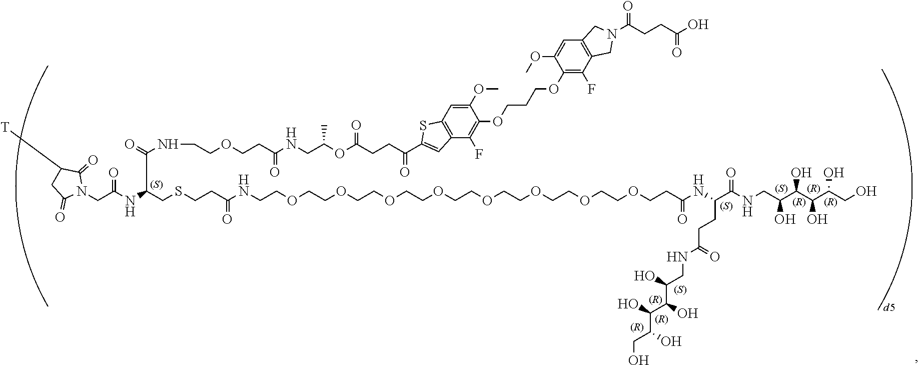

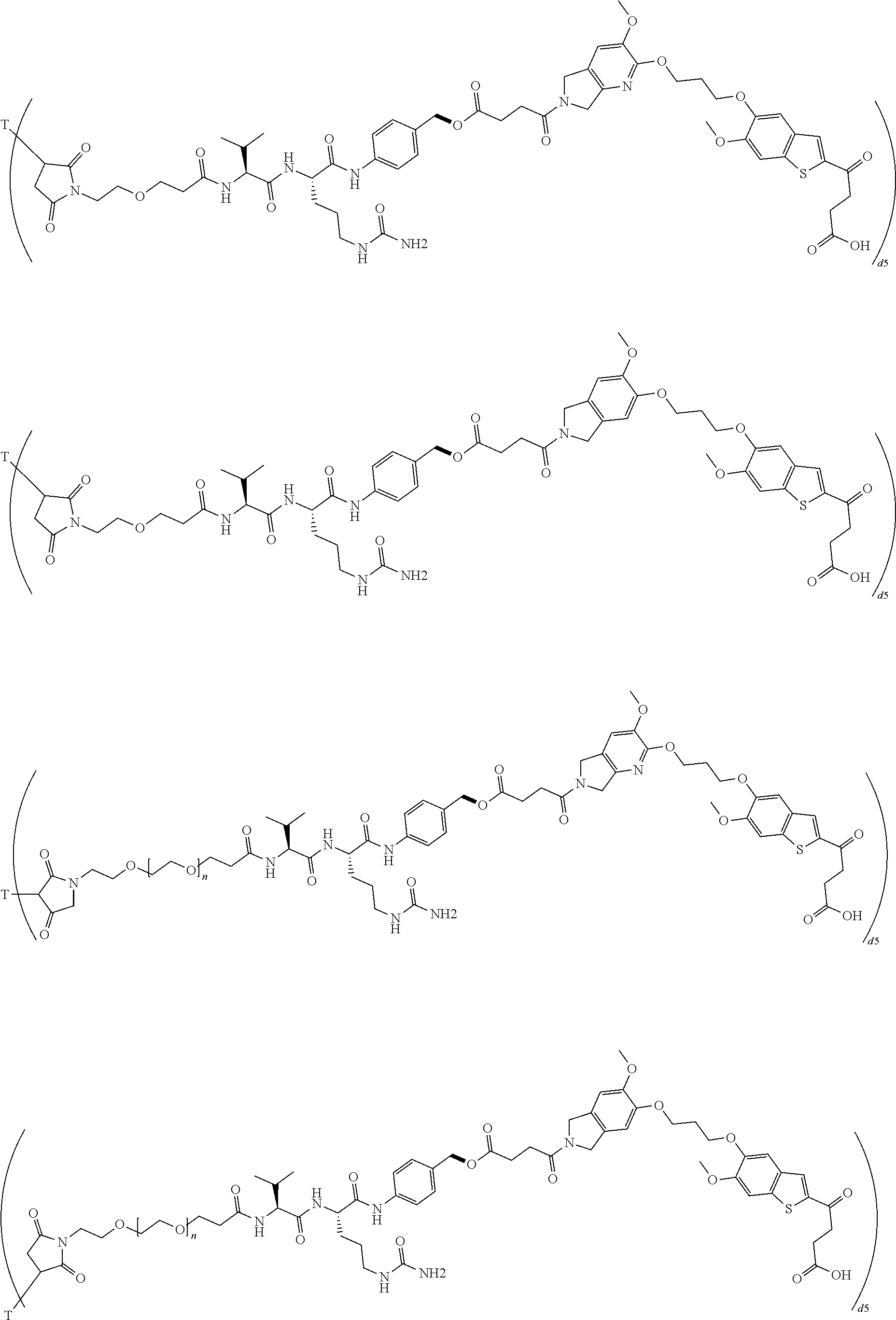

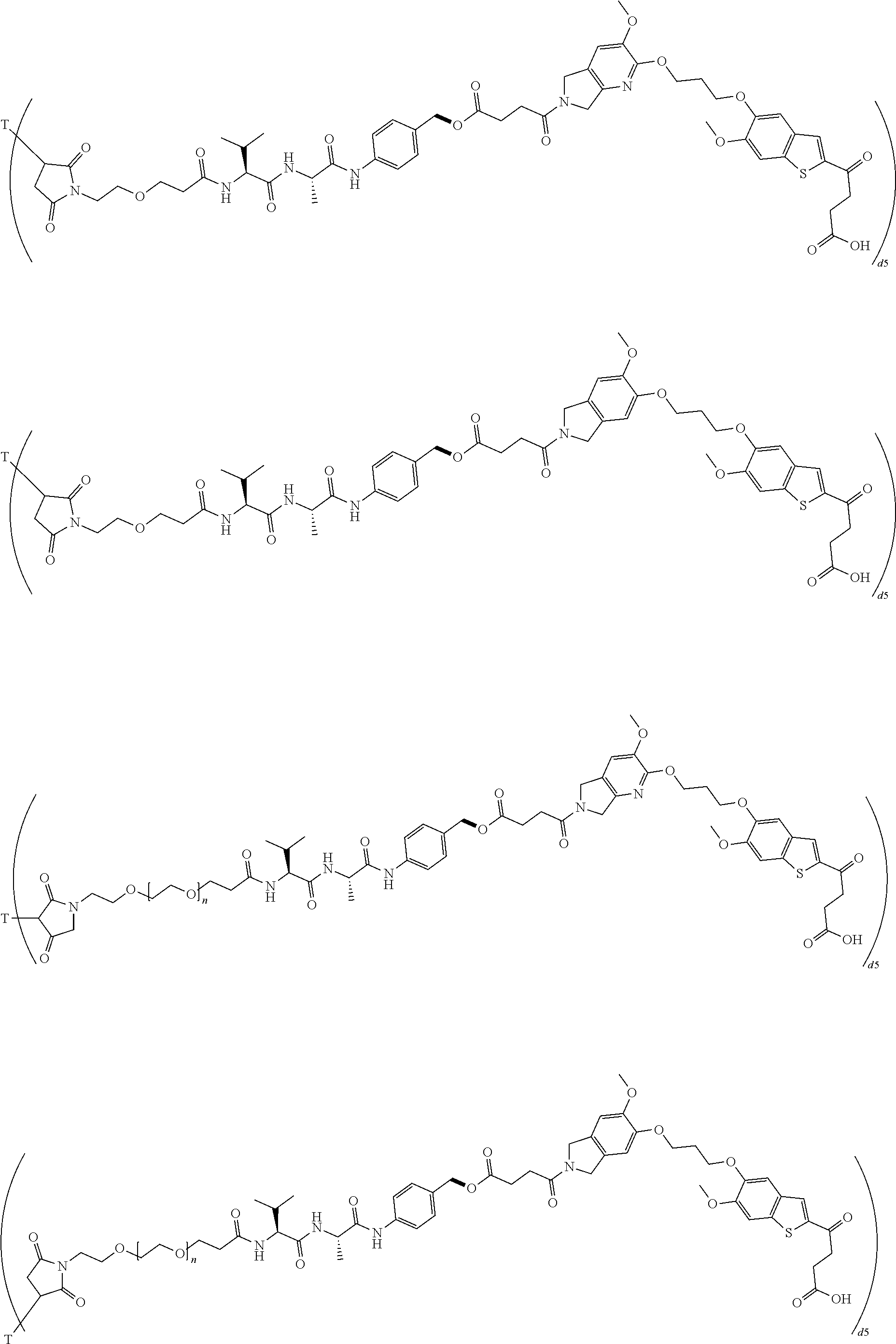

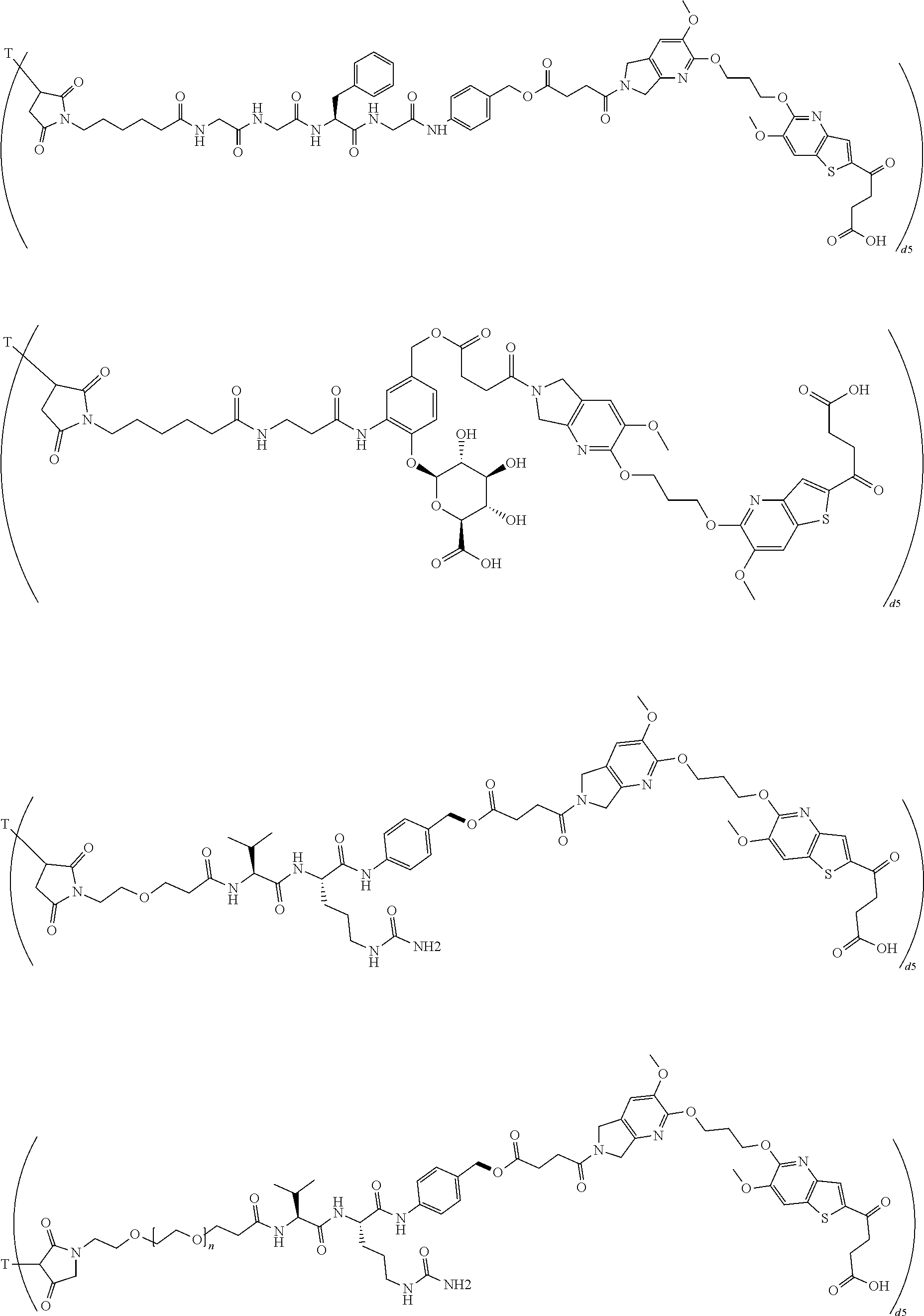

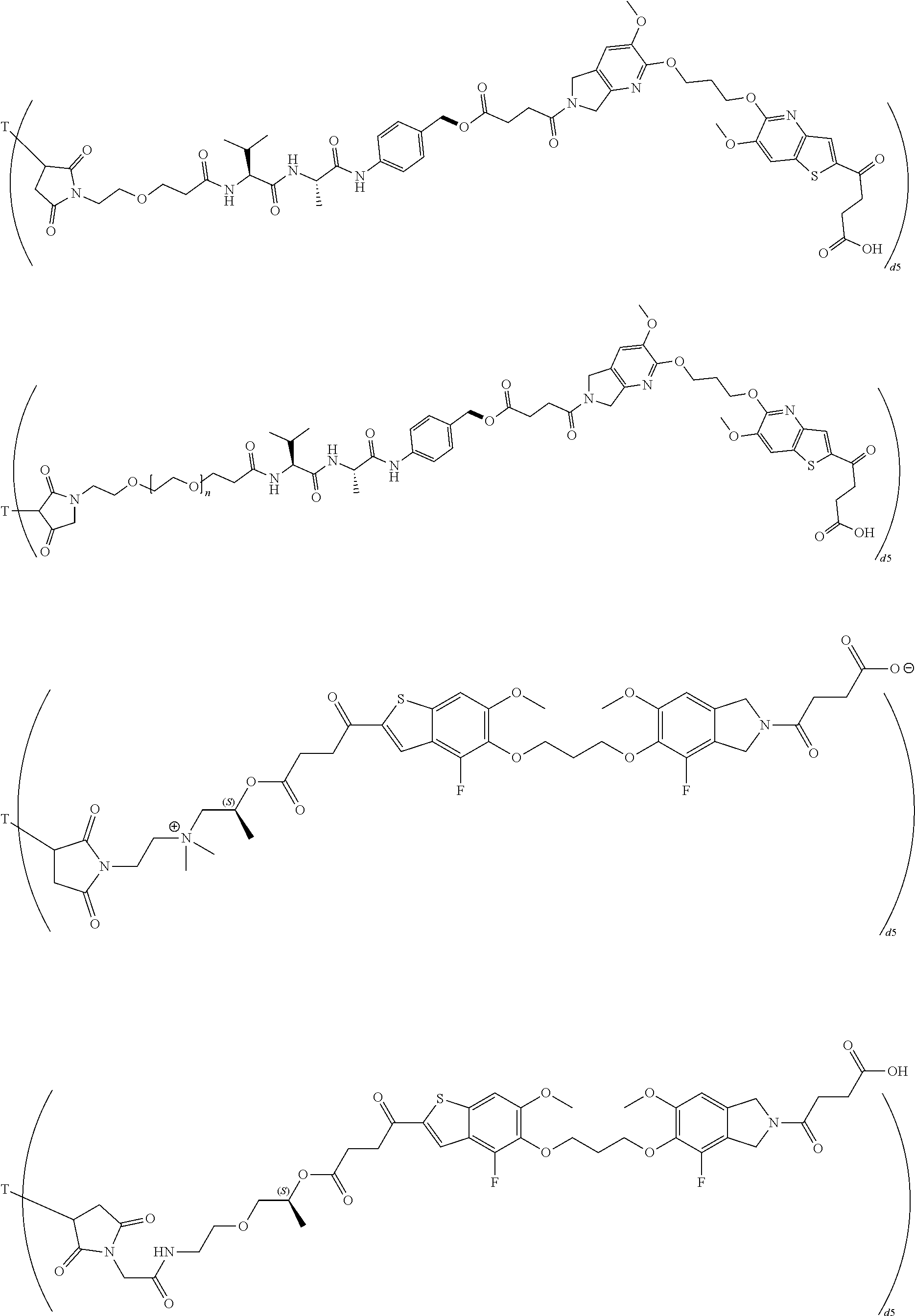

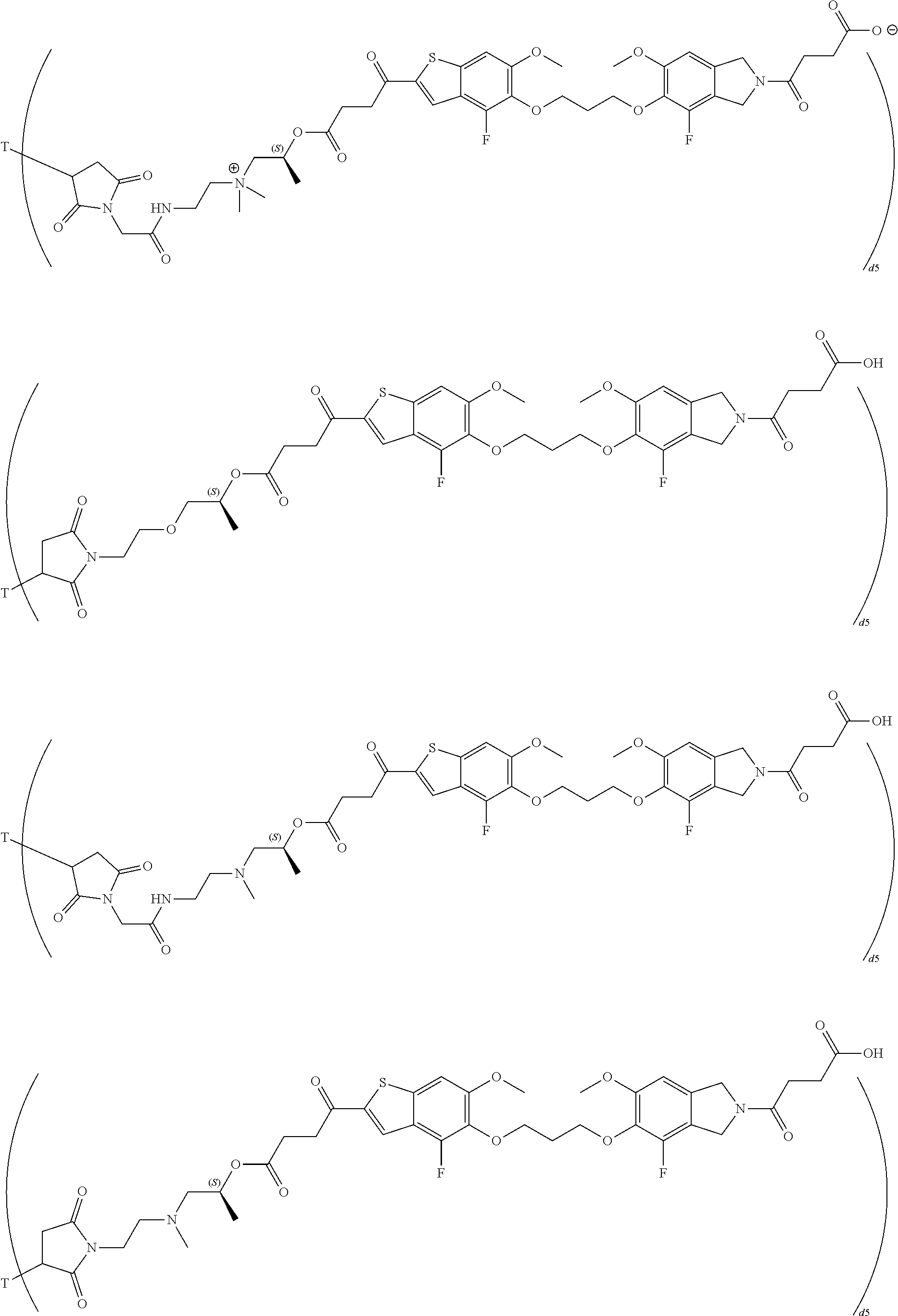

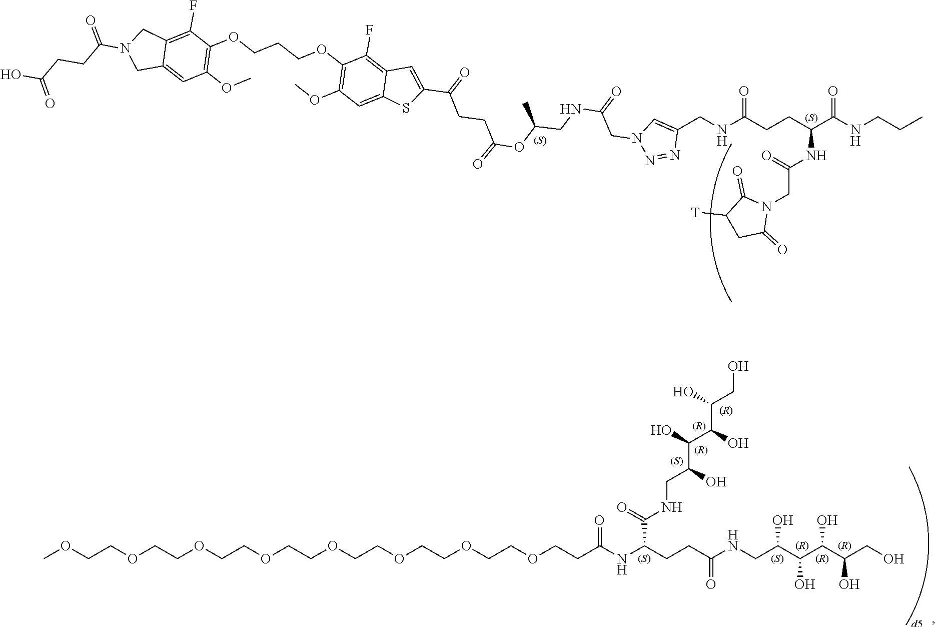

wherein d5 is an integer ranging from 1 to 20, such as from 6 to 10, such as 8.

In some embodiments, T directs the conjugates to specific tissues, cells, or locations in a cell. In some embodiments, the T can direct the conjugate in culture or in a whole organism, or both. In each case, the T may have a ligand that is present on the cell surface of the targeted cell(s) to which it binds with an effective specificity, affinity, and avidity. In some embodiments, the T targets the conjugate to tissues other than the liver. In some embodiments the T targets the conjugate to a specific tissue such as the liver, kidney, lung, or pancreas. The T can target the conjugate to a target cell such as a cancer cell, such as a receptor expressed on a cell such as a cancer cell, a matrix tissue, or a protein associated with cancer such as tumor antigen. Alternatively, cells comprising the tumor vasculature may be targeted. The Ts can direct the conjugate to specific types of cells such as specific targeting to hepatocytes in the liver as opposed to Kupffer cells. In some embodiments, Ts can direct the conjugate to cells of the reticular endothelial or lymphatic system, or to professional phagocytic cells such as macrophages or eosinophils. In some embodiments, the conjugate itself may also be an effective delivery system, without the need for specific targeting.

In some embodiments, the T can target the conjugate to a location within the cell, such as the nucleus, the cytoplasm, or the endosome, for example. In some embodiments, the T can enhance cellular binding toreceptors, or cytoplasmic transport to the nucleus and nuclear entry or release from endosomes or other intracellular vesicles.

In some embodiments, the T is an antibody, an antibody fragment, a protein, a peptide, or a peptide mimic. “Ts” refer to two or more T.

In some embodiments, the T is an antibody. In some embodiments, the T is an antibody fragment. In some embodiments, the T is a protein. In some embodiments, the T is a peptide. In some embodiments, the T is a peptide mimic.

In some embodiments, the antibody or antibody fragment is an antibody or antibody fragment wherein one or more amino acids of the corresponding parent antibody or antibody fragment (e.g., the corresponding wild type antibody or antibody fragment) are substituted with cysteines (e.g., engineered cysteine). In some embodiments, the parent antibody or antibody fragment may be wild type or mutated.

In some embodiments, the antibody or antibody fragment may be a mutated antibody or antibody fragment. In some embodiments, a monoclonal antibody known in the art is engineered to form the antibody.

In some embodiments, an antibody fragment (e.g., a Fab antibody fragment) known in the art is engineered to form the antibody fragment (e.g., a cysteine engineered Fab antibody fragment). In some embodiments, a single site mutation of a Fab gives a single residue in a Fab whereas a single site mutation in an antibody yields two amino acids in the resulting antibody due to the dimeric nature of the IgG antibody.

In some embodiments, the antibody or antibody fragment retains the antigen binding capability of its corresponding wild type antibody or antibody fragment. In some embodiments, the antibody or antibody fragment is capable of binding to the one or more antigens for its corresponding wild type antibody or antibody fragment.

In some embodiments, exemplary antibodies or antibodies derived from Fab, Fab2, scFv or camel antibody heavy-chain fragments specific to the cell surface markers, include, but are not limited to, 5T4, AOC3, ALK, AXL, B7-H4, C₂₄₂, C_{4.4}a, CA-125, CCL11, CCR 5, CD2, CD3, CD4, CD5, CD15, CA15-3, CD18, CD19, CA19-9, CDH6, CD20, CD22, CD23, CD25, CD28, CD30, CD31, CD33, CD37, CD38, CD40, CD41, CD44, CD44 v6, CD51, CD52, CD54, CD56, CD62E, CD62P, CD62 μL, CD70, CD73, CD74, CD79-B, CD80, CD125, CD103, CD138, CD141, CD147, CD152, CD 154, CD326, CEA, CEACAM-5, Claudin18.2, clumping factor, Clec9A, CSFR1, CTLA-4, CXCR2, DEC205, EGFR (HERI), ErbB1, ErbB2, ErbB3, EpCAM, EPHA2, EPHB2, EPHB4, FAP, FGFR (i.e. FGFR1, FGFR2, FGFR3, FGFR4), FLT3, fibronectin-EDB, folate receptor, GD2, GD3, GPNMB, GCC (GUCY2C), HGF, HER2, HER3, HMI.24, ICAM, ICOS-L, IGF-1 receptor, VEGFR1, EphA2, TRPV1, CFTR, gpNMB, CA9, Cripto, c-KIT, c-MET, ACE, APP, adrenergic receptor-beta2, Claudine 3, LIVI, LY6E, Mesothelin, MUC1, MUC13, NaPi2b, NOTCHI, NOTCH2, NOTCH3, NOTCH4, RON, RORI, PD-L1, PD-L2, PTK7, B7-H3, B7-B4, IL-2 receptor, IL-4 receptor, IL-13 receptor, TROP-2, frizzled-7, integrins (including I₄, ᵥβ₃, v35, 1v36, 114, 14P1, 1407, 151, 1604, 1-11bR3 integrins), IFN-J, IFN—U, IgE, IgE, IGF-1 receptor, IL-1, IL-12, IL-23, IL-13, IL-22, IL-4, IL-5, IL-6, interferon receptor, ITGB2 (CD18), LFA-1 (CD11a), CD11b, L-selectin (CD62L), mucin, myostatin, NCA-90, NGF, PDGFRI, phosphatidylserine, prostatic carcinoma cell, Pseudomonas aeruginosa, rabies, RANKL, respiratory syncytial virus, Rhesus factor, SLAMF7, sphingosine-1-phosphate, TAG-72, T-cell receptor, tenascin C, TGF-1, TGF- P 2, TGF-β, TNF-J, TRAIL-RI, TRAIL-R², tumor antigen CTAA16.88, VEGF-A, VEGFR2, vimentin, and the like.

In some embodiments the antibodies or antibody derived from Fab, Fab2, scFv or camel antibody heavy-chain fragments specific to the cell surface markers include CA-125, C₂₄₂, CD3, CD11b, CD19, CD22, CD25, CD30, CD31, CD33, CD37, CD40, CD44, CD51, CD54, CD56, CD62E, CD62P, CD62 μL, CD70, CD73, CD103, CD138, CD141, CD326, CEA, Claudin18.2, Clec9A, CSFR1, CTLA-4, DEC205, EGFR (HERI), ErbB2, ErbB3, FAP, fibronectin-EDB, folate receptor, IGF-1 receptor, GD3, GPNMB, HGF, HER2, VEGF-A, VEGFR2, VEGFR1, EphA2, EpCAM, 5T4, PTK7, TAG-72, tenascin C, TRPV1, CFTR, gpNMB, CA9, Cripto, ACE, APP, PDGFR 1, phosphatidylserine, prostatic carcinoma cells, adrenergic receptor-beta2, Claudine 3, mucin, MUC1, NaPi2b, B7H3, B7H4, C4.4a, CEACAM-5, MUC13, TROP-2, frizzled-7, Mesothelin, IL-2 receptor, IL-4 receptor, IL-13 receptor and integrins (including I ᵥβ 3, I ᵥ β₅, I ᵥ β₆, intergins), tenascin C, TRAIL-R², and vimentin.

In some embodiments, the antibodies are targeting cell surface markers for 5T4, CA-125, CEA, CDH6, CD3, CD11b, CD19, CD20, CD22, CD30, CD33, CD40, CD44, CD51, CD73, CD-103, CTLA-4, CEACAM5, Clec9A, CSFR1, DEC205, EpCAM, HER2, EGFR (HER1), FAP, fibronectin-EDB, folate receptor, GCC (GUCY2C), HGF, integrin 1-vP3, integrin 1-5p1, IGF-1 receptor, GD3, GPNMB, mucin, LIV1, LY6E, mesothelin, MUC1, MUC13, NaPi2b, PTK7, phosphatidylserine, prostatic carcinoma cells, PDGFR J, TAG-72, tenascin C, TRAIL-R², VEGF-A and VEGFR2.

In some embodiments, the antibodies include but are not limited to abagovomab, abciximab (REOPRO), adalimumab (HIJMIRA), adecatumumab, afelimomab, afutuzumab, alacizumab, ALD518, alemtuzumab (CAMPATH), altumomab, amatuximab, anatumomab, anrukinzumab, apolizumab, arcitumomab (CEA-SCAN), aselizumab, atezolizumab, atlizumab (tociiizumab, Actemra, RoActemra), atorolimumab, bapineuzumab, basiliximab (Simulect), bavituximab, bectumomab (LYMPHOSCAN), belimumab (BENLYSTA), benralizumab, bertilimumab, besilesomab (SCINITIMUN), bevacizumab (AVASTIN), biciromab (FIBRISCINT), bivatuzumab, blinatumomab, brentuximab, briakinumab, canakinumab (1 LA IS), cantuzumab, capromab, catumaxomab (REMOVAB), CC49, cedelizumab, certolizumab, cetuximab (ERBITUX), citatuzumab, cixutumumab, clenoliximab, clivatuzumab, conatuniumab, CR6261, dacetuzumab, daclizuniab (ZENAPAX), daratumumab, denosumab (PROLIA), detumomab, disitamab, dorlimomab, doriixizumab, ecrornexiniab, eculizumab (SOLIRIS), edobaconiab, edrecolomab (PANOREX), efalizumab (RAPTIVA), efungumab (MYCOGRAB), elotuzumab, efsilimomab, enlimomab, epitumomab, epratuzumab, erlizumab, ertumaxomab (REXOMUN), etaracizumab (ABEGRIN), exbivimrnab, fanolesomab (NEUTROSPEC), faralimomab, farJetuzumab, felvizumab, fezakinumab, figiturnurnab, fontolizumab (HuZAF), foravirumab, fresolimumab, galiximab, gantenerumab, gaviliniomab, gemtuzumab, girentuximab, glernbatumumab, golimumab (SIMPONI), gomiliximab, ibalizumab, ibritumomab, igovomab (INDIMACIS-125), imciromab (MYOSCINT), infliximab (REMICADE), intetumumab, inolimomab, inotuzumab, ipilimumab, irafumumab, keliximab, labetuzumab (CEA-CIDE), lebrikizumab, lemalesomab, lerdelimumab, lexatumumab, libivirumab, lintuzumab, lucatumumab, lumiliximab, mapatumumab, maslimomab, matuzumab, mepolizumab (BOSATRIA), metelimumab, milatuzumab, minretumomab, mitumomab, morolimumab, rnotavizumab (NUMAX), muromonab-CD3 (ORTHOCLONE OKT3), nacofomab, naptumomab, natalizumab (TYSABRI), nebacumab, necitumumab, nerelimomab, nimotuzumab (THERACIM), nofetumomab, ocrelizumab, odulimomab, ofatumumab (ARZERRA), olaratumab, omalizumab (XOLAIR), o tecizumab, oportuzumab, oregovomab (OVAREX), otelixizumab, pagibaximab, palivizumab (SYNAGIS), panitumumab (VECTIBIX), panobacumab, pascolizumab, pemtumomab (THERA GYN), pertuzumab (OMNITARG), pexelizumab, pintumomab, priliximab, pritumumab, PRO 140, rafivirumab, rarnucirumab, ranibizumab (LUCENTIS), raxibacumab, regavirumab, reslizumab, rilotumumab, rituximab (RITUXAN), robatumumab, rontalizumab, rovelizumab (LEUKARREST), ruplizumab (ANTOVA), satumomab pendetide, sevirumab, sibrotuzumab, sifalimumab, siltuximab, siplizumab, sofanezumab, sonepcizumab, sontuzumab, stamulumab, sulesomab (LEUKOSCAN), tacatuzumab (AFP-CIDE), fetraxefan, tadocizumab, talizumab, tanezumab, taplitumomab paptox, tefibazumab (AUREXIS), telimomab, tenatumomab, teneliximab, teplizumab, TGN1412, ficilimumab (tremelimumab), tigatuzumab, TNX-650, tocilizumab (atlizumab, ACTEMRA), toralizumab, tositumomab (BEXXAR), trastuzumab (HERCEPITN), tremelimumab, tucotuzumab, tuvirumab, urtoxazumab, ustekinumab (STELERA), vapaliximab, vedolizumab, veltuzumab, vepalimomab, visilizumab (NUVIQN), volociximab (HUMASPECT), votumumab, zalutumumab (HuMEX-EGFr), zanolimumab (HuMAX-CD4), ziralimumab and zolimomab.

In some embodiments, the antibodies include but are not limited to, abagovomab, adecatumumab, alacizumab, altumomab, anatumomab, arcitumomab, atezolizumab, bavituximab, bevacizumab (AVASTIN®), bivatuzumab, blinatumomab, brentuximab, cantuzumab, catumaxomab, capromab, cetuximab, citatuzumab, clivatuzumab, conatumumab, dacetuzumab, disitamab, edrecolomab, epratuzumab, ertumaxomab, etaracizumab, farletuzumab, figitumumab, gemtuzumab, glembatumumab, ibritumomab, igovomab, intetumumab, inotuzumab, labetuzumab, lexatumumab, lintuzumab, lucatumumab, matuzumab, mitumomab, naptumomab estafenatox, necitumumab, oportuzumab, oregovomab, panitumumab, pemtumomab, pertuzumab, pritumumab, rituximab (RITUXAN®), rilotumumab, robatumumab, satumomab, sibrotuzumab, taplitumomab, tenatumomab, tenatumomab, ticilimumab (tremelimumab), tigatuzumab, trastuzumab (HERCEPTIN®), tositumomab, tremelimumab, tucotuzumab, celmoleukin, volociximab, and zalutumumab.

In some embodiments the antibody targeting cell surface markers for HER2 is trastuzumab, pertuzumab or disitamab and for EGFR (HER1) the antibody is cetuximab or panitumumab; and for CD20 the antibody is rituximab and for VEGF-A is bevacizumab and for CD-22 the antibody is epratuzumab or veltuzumab and for CEA the antibody is labetuzumab.

In some embodiments, the antibody targeting CD73 has a heavy chain of the amino acid sequence as shown in SEQ ID NO: 1 and a light chain of the amino acid sequence as shown in SEQ ID NO: 2. In some embodiments, the antibody targeting CD73 has a heavy chain of the amino acid sequence as shown in SEQ ID NO: 3 and a light chain of the amino acid sequence as shown in SEQ ID NO: 2. In some embodiments, the antibodies disclosed herein comprise a heavy chain variable region having an amino acid sequence at least 85%, 86%, 87% 88%, 89%, 90%, 91%, 92%, 93%, 94%, 95%, 96%, 97% 98%, 99% or more identical to a sequence selected from SEQ ID NOs: 1 and a light chain variable region having an amino acid sequence at least 85%, 86%, 87% 88%, 89%, 90%, 91%, 92%, 93%, 94%, 95%, 96%, 97% 98%, 99% or more identical to a sequence selected from SEQ ID NOs: 2. In some embodiments, the antibodies disclosed herein comprise a heavy chain variable region having an amino acid sequence at least 85%, 86%, 87% 88%, 89%, 90%, 91%, 92%, 93%, 94%, 95%, 96%, 97% 98%, 99% or more identical to a sequence selected from SEQ ID NOs: 3 and a light chain variable region having an amino acid sequence at least 85%, 86%, 87% 88%, 89%, 90%, 91%, 92%, 93%, 94%, 95%, 96%, 97% 98%, 99% or more identical to a sequence selected from SEQ ID NOs: 2.

In some embodiments, the antibody targeting c-Met has a heavy chain of the amino acid sequence as shown in SEQ ID NO: 4 and a light chain of the amino acid sequence as shown in SEQ ID NO: 5. In some embodiments, the antibody targeting c-Met has a heavy chain of the amino acid sequence as shown in SEQ ID NO: 8 and a light chain of the amino acid sequence as shown in SEQ ID NO: 9. In some embodiments, the antibodies disclosed herein comprise a heavy chain variable region having an amino acid sequence at least 85%, 86%, 87% 88%, 89%, 90%, 91%, 92%, 93%, 94%, 95%, 96%, 97% 98%, 99% or more identical to a sequence selected from SEQ ID NOs: 4 and a light chain variable region having an amino acid sequence at least 85%, 86%, 87% 88%, 89%, 90%, 91%, 92%, 93%, 94%, 95%, 96%, 97% 98%, 99% or more identical to a sequence selected from SEQ ID NOs: 5. In some embodiments, the antibodies disclosed herein comprise a heavy chain variable region having an amino acid sequence at least 85%, 86%, 87% 88%, 89%, 90%, 91%, 92%, 93%, 94%, 95%, 96%, 97% 98%, 99% or more identical to a sequence selected from SEQ ID NOs: 8 and a light chain variable region having an amino acid sequence at least 85%, 86%, 87% 88%, 89%, 90%, 91%, 92%, 93%, 94%, 95%, 96%, 97% 98%, 99% or more identical to a sequence selected from SEQ ID NOs: 9.

In some embodiments, the antibody targeting PD-L1 is atezolizumab.

Exemplary peptides or peptide mimics include integrin targeting peptides (RGD peptides), LHRH receptor targeting peptides, ErbB2 (HER2) receptor targeting peptides, prostate specific membrane bound antigen (PSMA) targeting peptides, lipoprotein receptor LRP1 targeting, ApoE protein derived peptides, ApoA protein peptides, somatostatin receptor targeting peptides, chlorotoxin derived peptides, and bombesin.

In some embodiments, the peptides or peptide mimics are LHRH receptor targeting peptides and ErbB2 (HER2) receptor targeting peptides

Exemplary proteins comprise insulin, transferrin, fibrinogen-gamma fragment, thrombospondin, claudin, apolipoprotein E, Affibody molecules such as, for example, ABY-025, ankyrin repeat proteins, ankyrin-like repeats proteins and synthetic peptides.

In some embodiments, the conjugates comprise broad spectrum cytotoxins in combination with cell surface markers for HER2, such as, for example, pertuzumab trastuzumab or disitamab; for EGFR such as cetuximab and panitumumab; for CEA such as labetuzumab; for CD20 such as rituximab; for VEGF-A such as bevacizumab; or for CD-22 such as epratuzumab or veltuzumab.

In some embodiments, the conjugates used in the disclosure comprise combinations of two or more Ts, such as, for example, combination of bispecific antibodies targeting the EGF receptor (EGFR) on tumor cells and to CD3 and CD28 on T cells; combination of antibodies or antibody derived from Fab, Fab2, scFv or camel antibody heavy-chain fragments and peptides or peptide mimetics; combination of antibodies or antibody derived from Fab, Fab2, scFv or camel antibody heavy-chain fragments and proteins; combination of two bispecific antibodies such as CD3-CD19 plus CD28-CD22 bispecific antibodies.

In some embodiments, the targeting moiety T comprises an antibody, an antibody fragment, a nucleic acid based molecule, a carbohydrate, a peptide, or a modified peptide, in particular an antibody or an antigen-binding fragment, which is designed to target the Human Epidermal Growth Factor Receptor (EGFR), a plasminogen activator, a cytotoxic T-lymphocyte associated antigen (CTLA) such as CTLA-4, PD-1, PD-L1, KIR, TIM3, VISTA, TIGIT, LAG3, OX40, RORI, ROR2, vascular endothelial growth factor (VEGF), fibroblast growth factor receptor (FGFR), platelet-derived growth factor (PDGF), transforming growth factor (TGF), neurotrophic factors, a nerve growth factor, platelet-derived growth factor (PDGF), interleukin receptors, transforming growth factor (TGF), estrogen receptor, progesterone receptor, c-Kit, cMET, ErbB2/Her2, ErbB3/Her3, ErbB4/Her4, CD3, CD20, CD22, CD30, CD33, CD40, CD47, CD79, CD123, CD133, CD166, CD137, the mesothelin protein, EpCAM, FLT3, PSMA, PSCA, STEAP, CEA, folate receptor, the CD39/CD73 receptors, adenosine receptors, SLC34A2 gene product, the EphA2 tyrosine kinase, the MucI/Muc16 cell-surface antigens, ALK, AFP, bcr-Abl, PAP.

In some embodiments, the conjugates used in the disclosure comprise Ts are antibodies such as Trastuzumab, Disitamab, Cetuximab, Rituximab, Bevacizumab, Epratuzumab, Veltuzumab, Labetuzumab, Atezolizumab. In some embodiments, the conjugates used in the disclosure comprise Ts are antibodies against antigens, such as, for example, β₇—H4, β₇—H3, CD11b, CD103, CA125, CDH6, CD33, CD73, Claudin18.2, CXCR2, CEACAM5, Clec9A, CSFR1, DEC205, EGFR, FAP, fibronectin-EDB, FGFRI, FGFR2, FGFR3, FGFR4, GCC (GUCY2C), HER2, LIV1, LY6E, NaPi2b, c-Met, mesothelin, NOTCHI, NOTCH2, NOTCH3, NOTCH4, PD-LI, PTK7, c-Kit, MUC1, MUC13. and 5T4.

In some embodiments, the conjugates of the disclosure comprise Ts which are CSRFI, CD11b, DEC205, clec9A, CD103, β₇H4, mesothelin, PTK7, Ly6E, FAP, fibronectin-EDB, Her-2 or NaPi2b antibodies.

In some embodiments, the conjugate of the disclosure has the following structure:

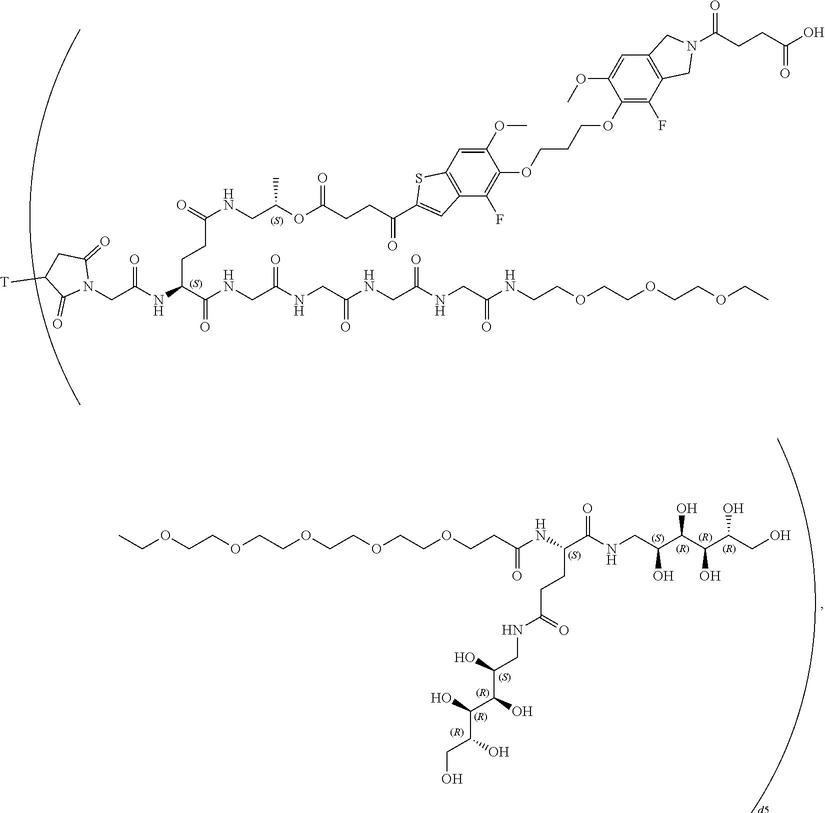

wherein T is an anti-HER2 antibody, such as Trastuzumab or Disitamab, and d5 is an integer of 6-8, such as 7 or 8.

In some embodiments, the conjugate of the disclosure has the following structure:

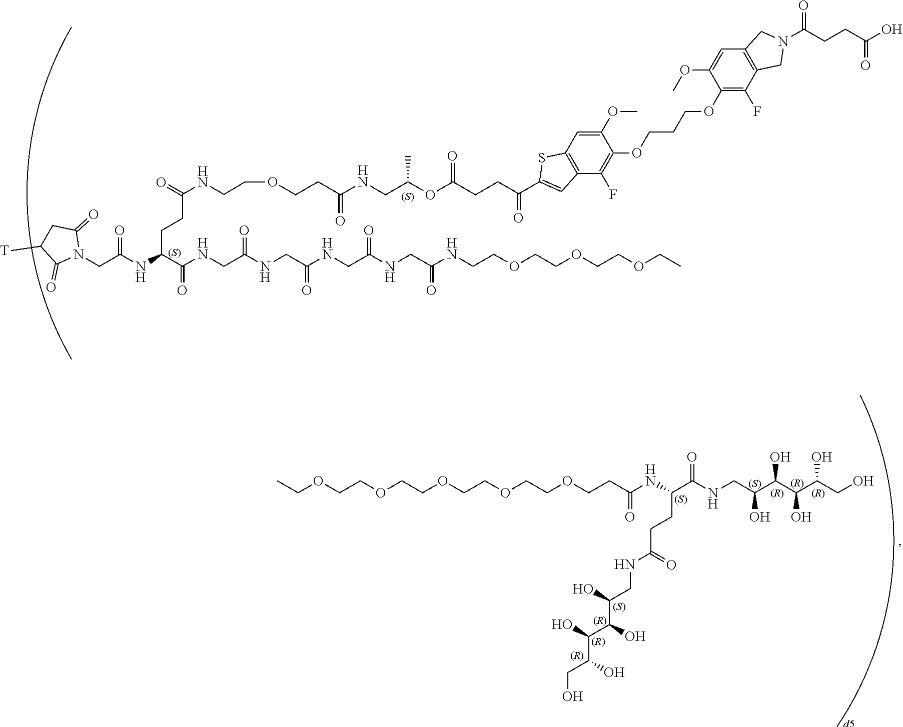

wherein T is an anti-HER2 antibody, such as Trastuzumab or Disitamab, and d5 is an integer of 6-8, such as 7 or 8.

In some embodiments, the conjugate of the disclosure has the following structure:

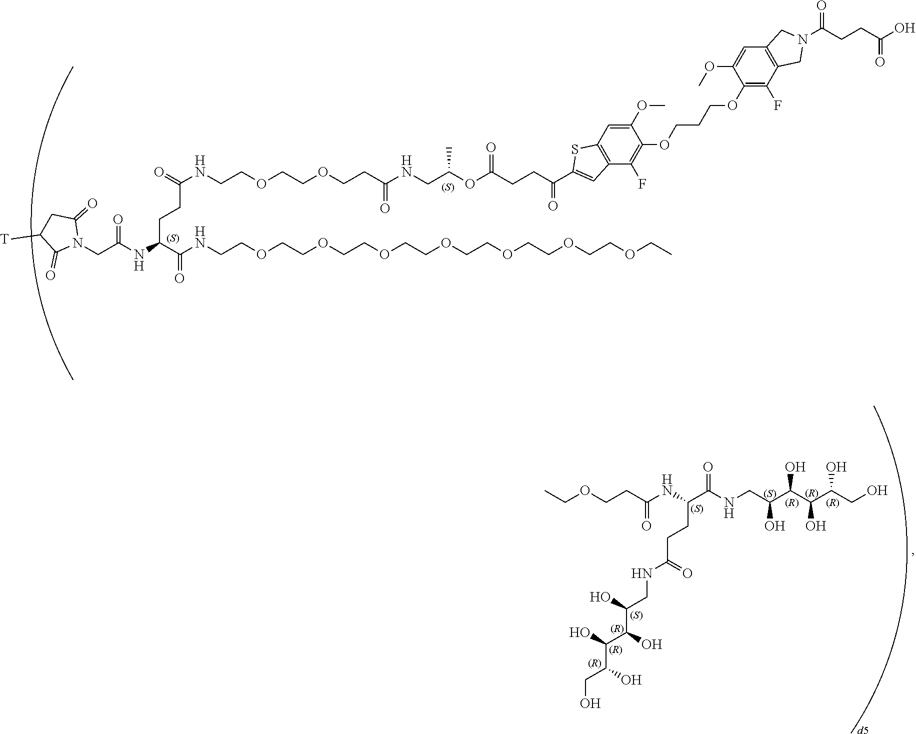

wherein T is an anti-HER2 antibody, such as Trastuzumab or Disitamab, and d5 is an integer of 6-8, such as 7 or 8.

In some embodiments, the conjugate of the disclosure has the following structure:

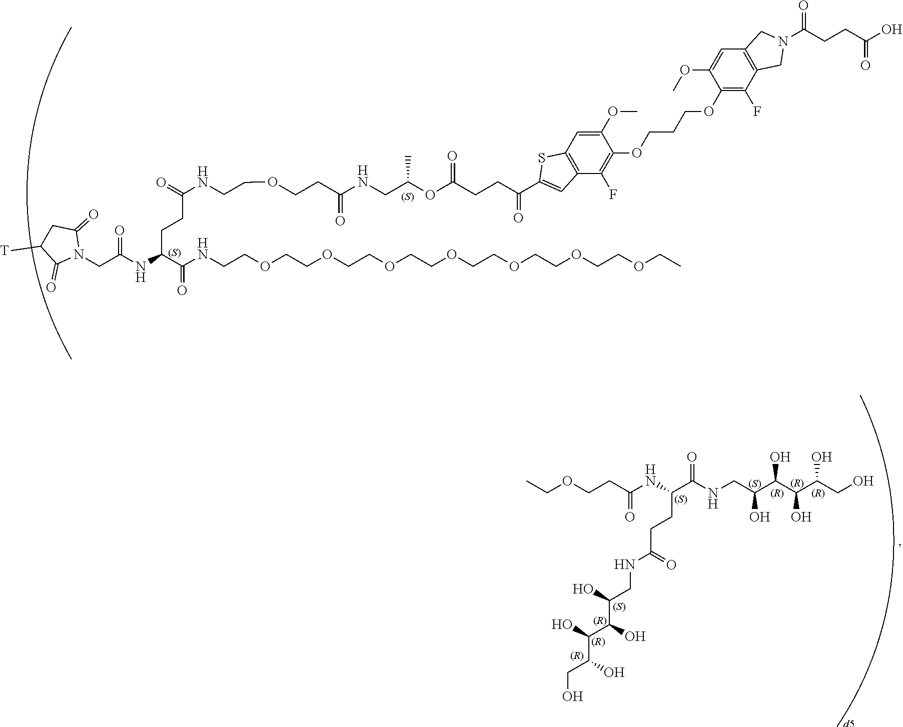

wherein T is an anti-HER2 antibody, such as Trastuzumab or Disitamab, and d5 is an integer of 6-8, such as 7 or 8.

In some embodiments, the conjugate of the disclosure has the following structure:

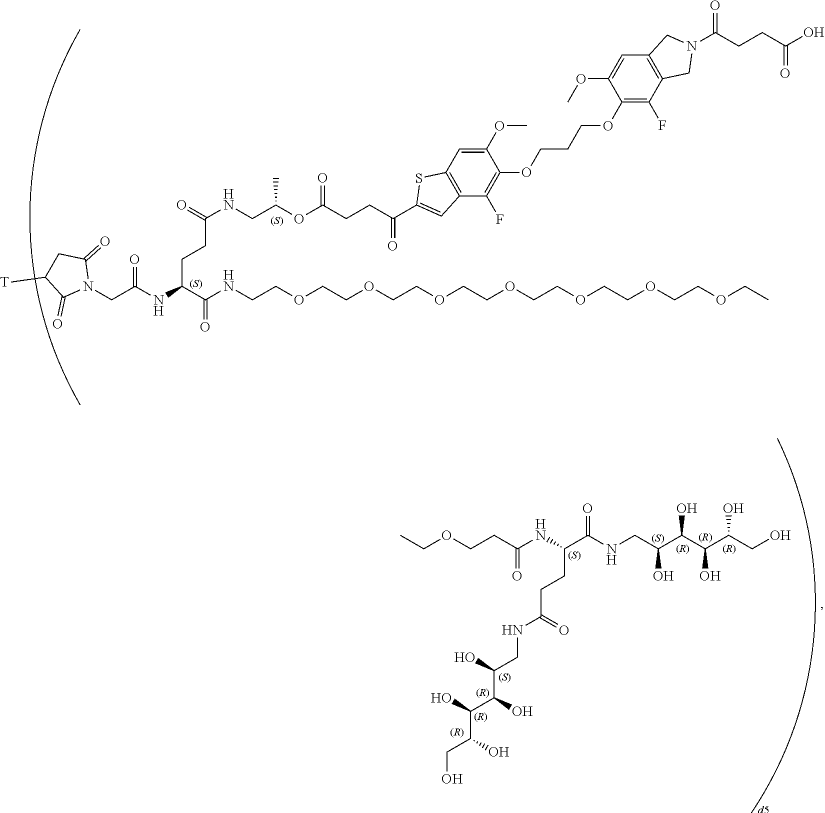

wherein T is an anti-HER2 antibody, such as Trastuzumab or Disitamab, and d5 is an integer of 6-8, such as 7 or 8.

In some embodiments, the conjugate of the disclosure has the following structure:

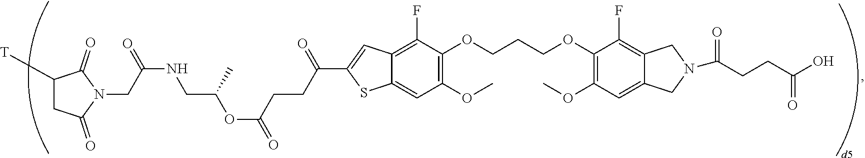

wherein T is an anti-HER2 antibody, such as Trastuzumab or Disitamab, and d5 is an integer of 6-8, such as 7 or 8.

In some embodiments, the conjugate of the disclosure has the following structure:

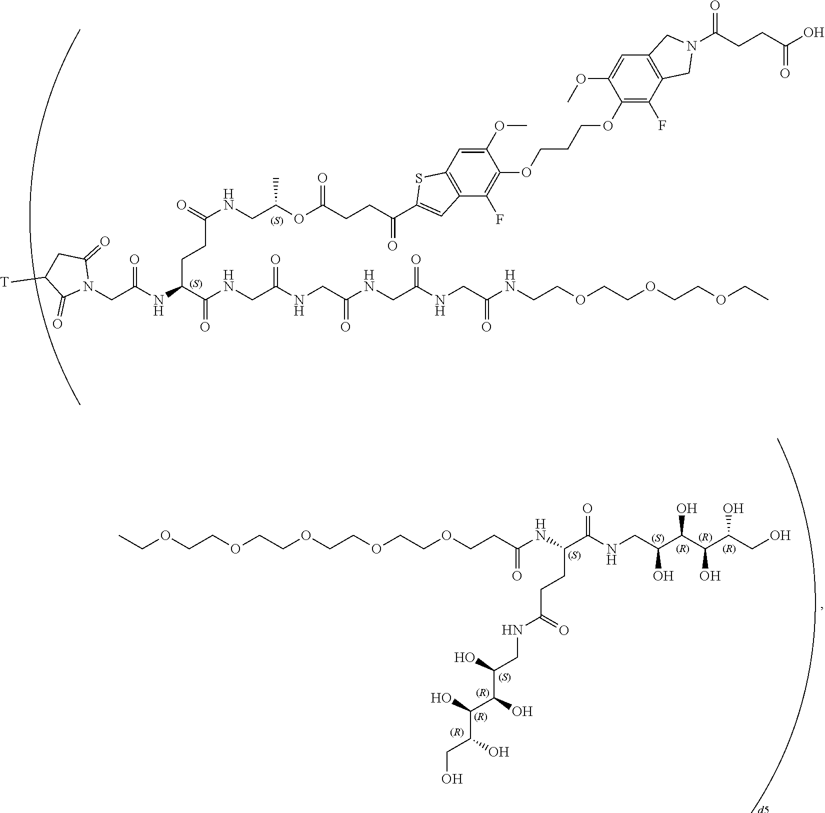

wherein T is anti-CD73 antibody having a heavy chain of the amino acid sequence as shown in SEQ ID NO: 1 or 3 and a light chain of the amino acid sequence as shown in SEQ ID NO: 2, and d5 is an integer of 6-8, such as 7 or 8.

(SEQ ID NO: 1)
QVQLVQSGAEVKKPGASVKVSCKASGYTFTSYWMHWVRQAPGQGLEWMG
YINPSSGYTKSNQKFKDRVTMTADTSTSTAYMELSSLRSEDTAVYYCGR
WLLSAWFAYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK
(SEQ ID NO: 2)
DIQMTQSPSSLSASVGDRVTITCKASQDINTYLSWFQQKPGKSPKSLIY
RSNILVDGVPSRFSGSGSGQDYTLTISSLQPEDFAIYYCLQYDDFPYTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
(SEQ ID NO: 3)
QVQLVQSGAEVKKPGASVKVSCKASGYTFTSYWMHWVRQAPGQGLEWMG
YINPSSGYTKSNQKFKDRVTMTADTSTSTAYMELSSLRSEDTAVYYCGR
WLLSAWFAYWGQGTLVTVSSASTKGPSVFPLAPCSRSTSESTAALGCLV
KDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPK
DTLMISRTPEVTCVVVDVSHEDPEVQFNWYVDGVEVHNAKTKPREEQFN
STFRVVSVLTVVHQDWLNGKEYKCKVSNKGLPAPIEKTISKTKGQPREP
QVYTLPPSREEMTKNQVSLTCLVKGFYPSDIAVEWESNGQPENNYKTTP
PMLDSDGSFFLYSKLTVDKSRWQQGNVFSCSVMHEALHNHYTQKSLSLS
PGK

In some embodiments, the conjugate of the disclosure has the following structure:

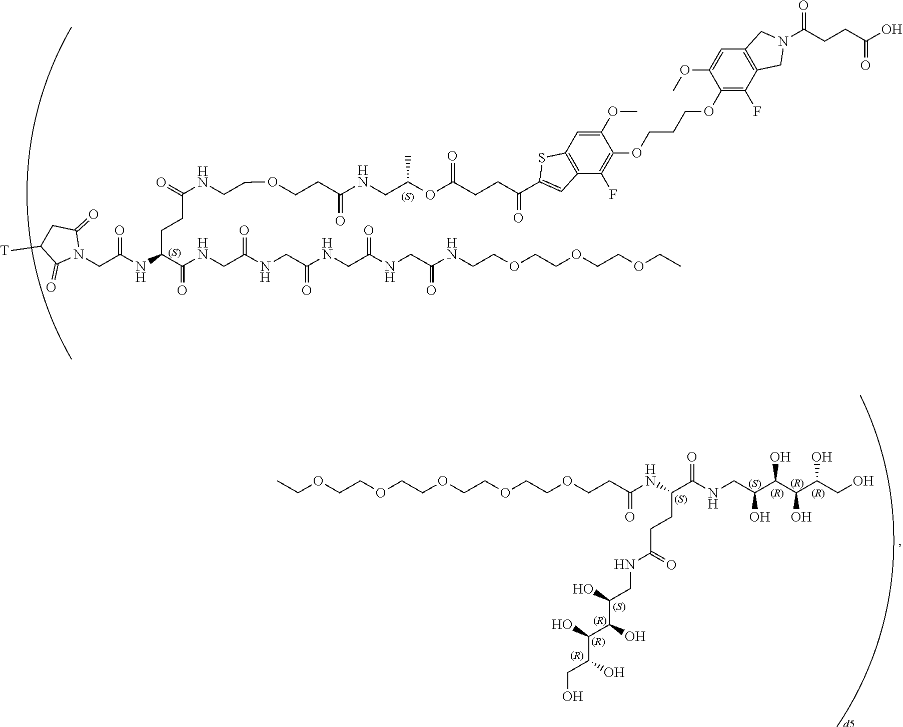

wherein T is anti-CD73 antibody having a heavy chain of the amino acid sequence as shown in SEQ ID NO: 1 or 3 and a light chain of the amino acid sequence as shown in SEQ ID NO: 2, and d5 is an integer of 6-8, such as 7 or 8.

In some embodiments, the conjugate of the disclosure has the following structure:

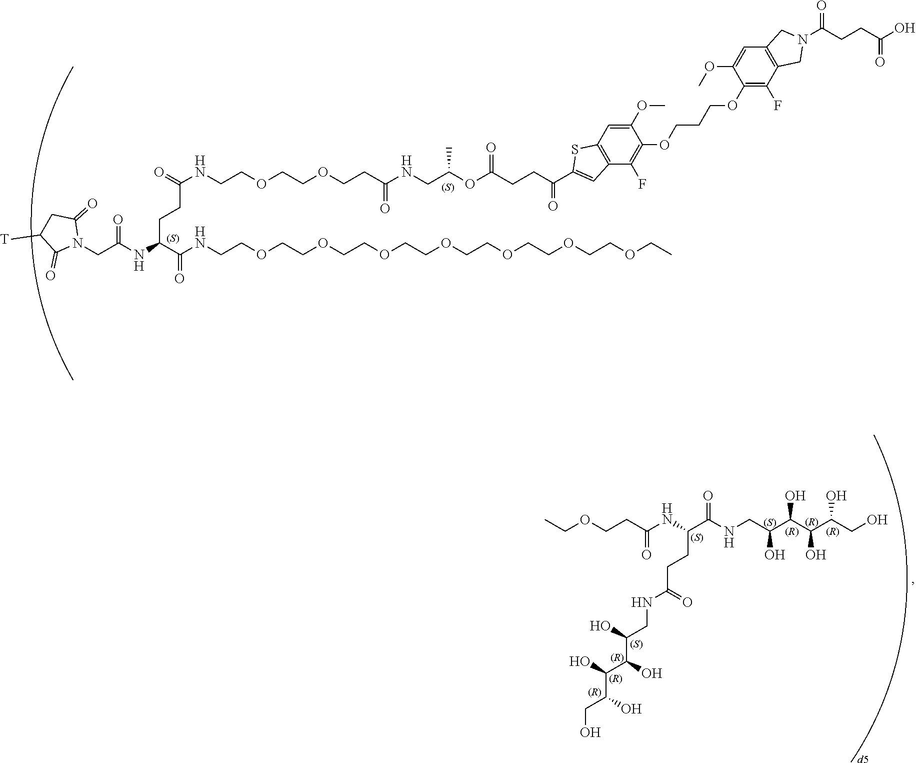

wherein T is anti-CD73 antibody having a heavy chain of the amino acid sequence as shown in SEQ ID NO: 1 or 3 and a light chain of the amino acid sequence as shown in SEQ ID NO: 2, and d5 is an integer of 6-8, such as 7 or 8.

In some embodiments, the conjugate of the disclosure has the following structure:

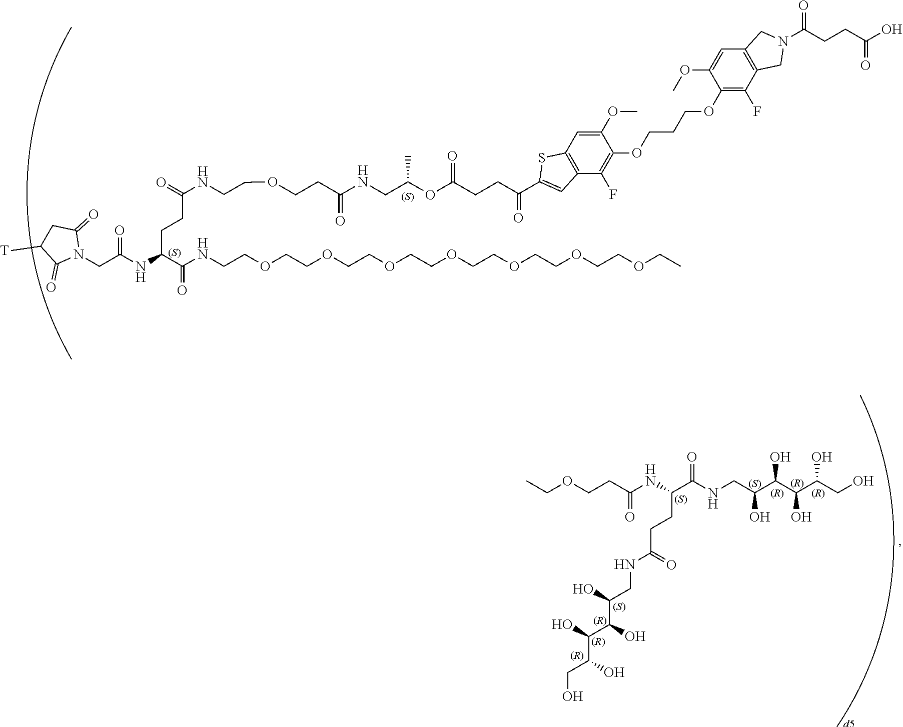

wherein T is anti-CD73 antibody having a heavy chain of the amino acid sequence as shown in SEQ ID NO: 1 or 3 and a light chain of the amino acid sequence as shown in SEQ ID NO: 2, and d5 is an integer of 6-8, such as 7 or 8.

In some embodiments, the conjugate of the disclosure has the following structure:

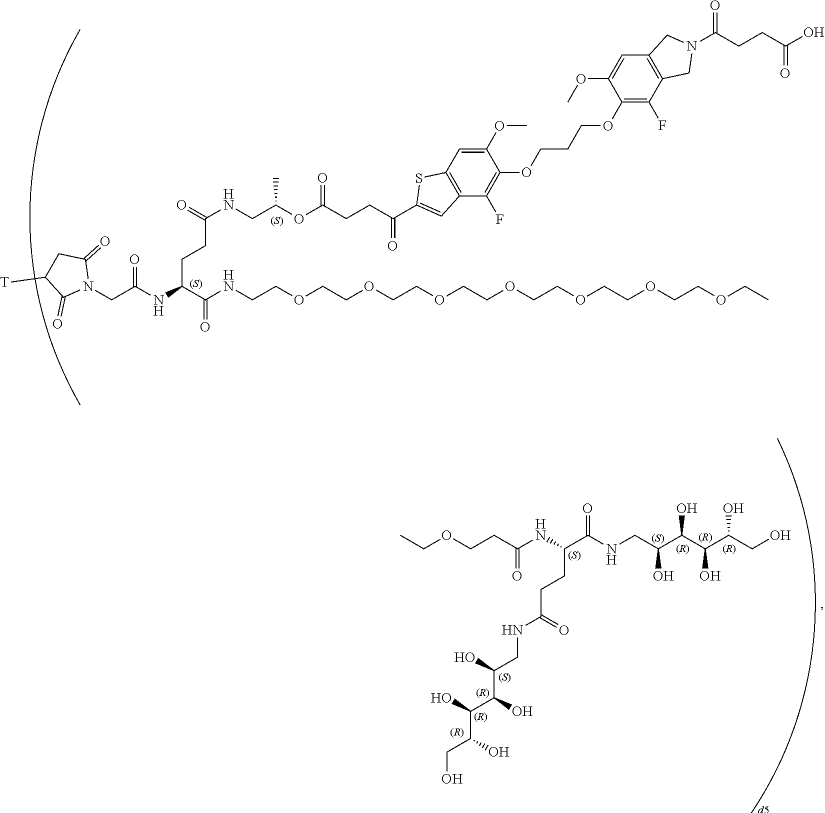

wherein T is anti-CD73 antibody having a heavy chain of the amino acid sequence as shown in SEQ ID NO: 1 or 3 and a light chain of the amino acid sequence as shown in SEQ ID NO: 2, and d5 is an integer of 6-8, such as 7 or 8.

In some embodiments, the conjugate of the disclosure has the following structure:

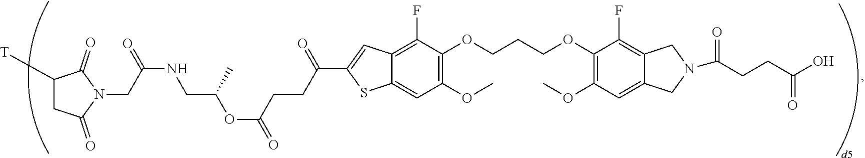

wherein T is anti-CD73 antibody having a heavy chain of the amino acid sequence as shown in SEQ ID NO: 1 or 3 and a light chain of the amino acid sequence as shown in SEQ ID NO: 2, and d5 is an integer of 6-8, such as 7 or 8.

In some embodiments, the conjugate of the disclosure has the following structure:

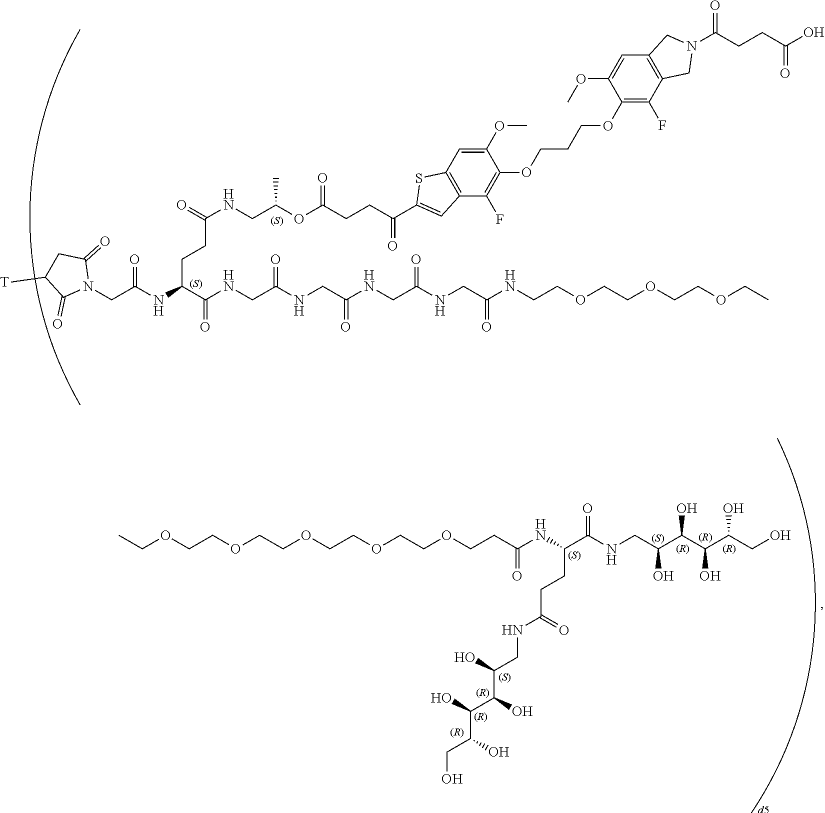

wherein T is an anti-PD-L1 antibody, such as Atezolizumab, and d5 is an integer of 6-8, such as 7 or 8.

In some embodiments, the conjugate of the disclosure has the following structure:

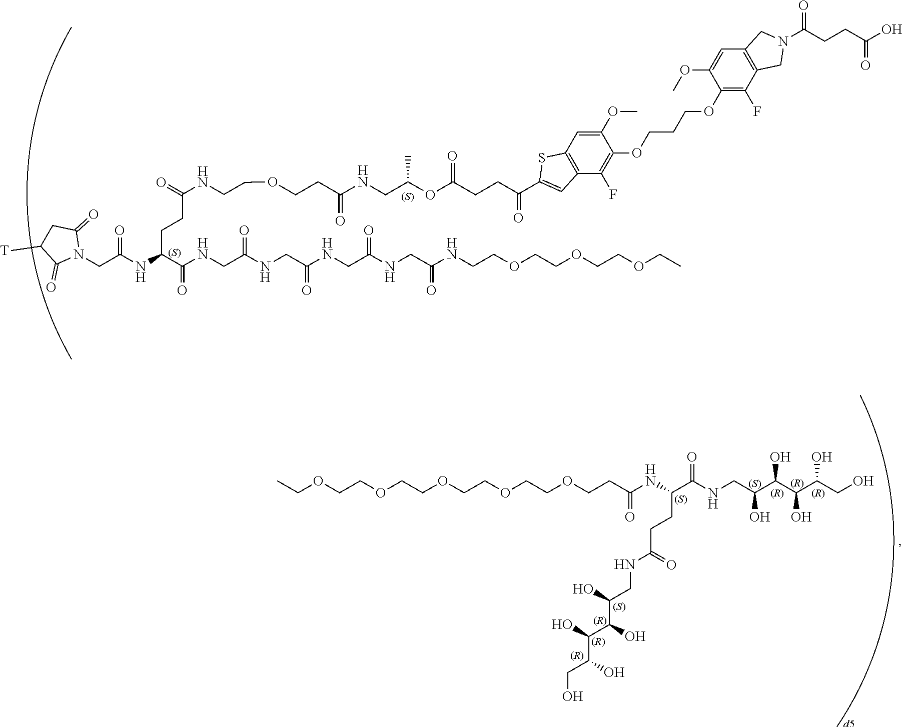

wherein T is an anti-PD-L1 antibody, such as Atezolizumab, and d5 is an integer of 6-8, such as 7 or 8.

In some embodiments, the conjugate of the disclosure has the following structure:

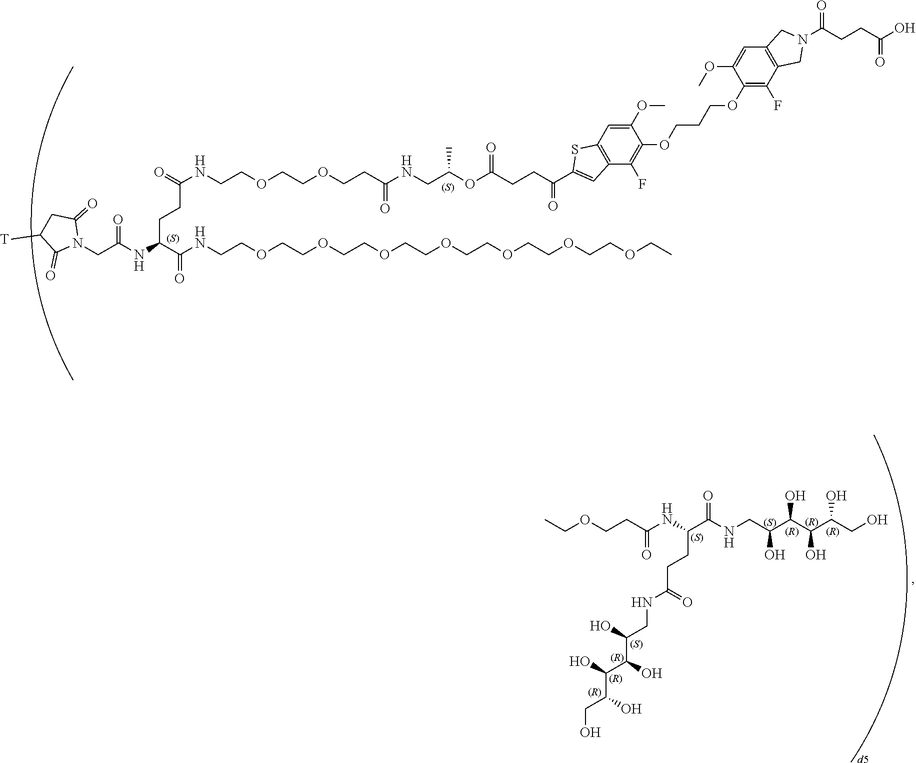

wherein T is an anti-PD-L1 antibody, such as Atezolizumab, and d5 is an integer of 6-8, such as 7 or 8.

In some embodiments, the conjugate of the disclosure has the following structure:

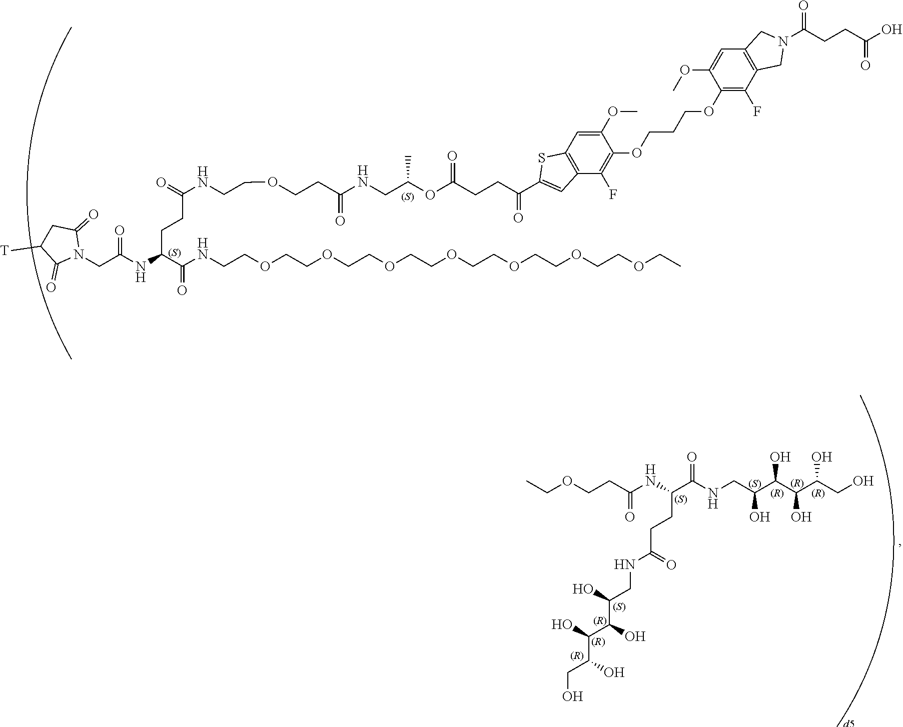

wherein T is an anti-PD-L1 antibody, such as Atezolizumab, and d5 is an integer of 6-8, such as 7 or 8.

In some embodiments, the conjugate of the disclosure has the following structure:

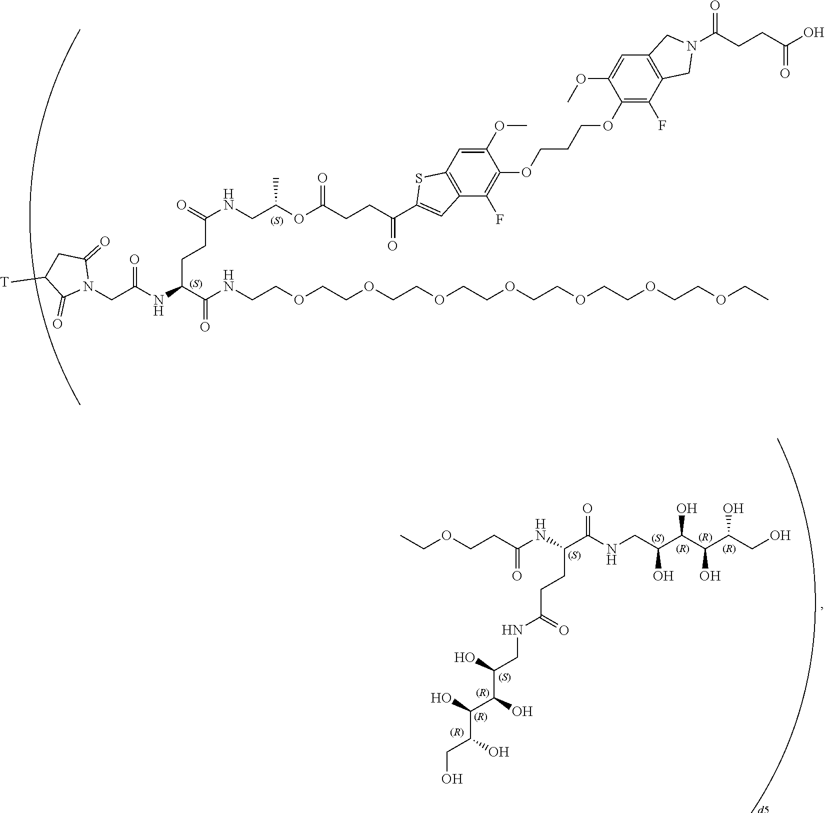

wherein T is an anti-PD-L1 antibody, such as Atezolizumab, and d5 is an integer of 6-8, such as 7 or 8.

In some embodiments, he_conjugate_of_the disclosure has the followingstructure

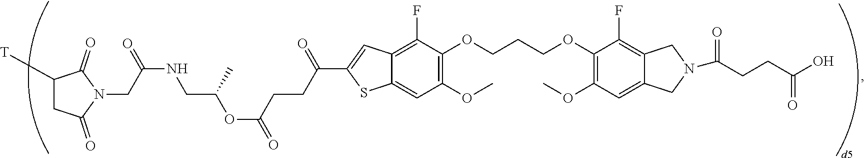

wherein T is an anti-PD-L1 antibody, such as Atezolizumab, and d5 is an integer of 6-8, such as 7 or 8.

In some embodiments, the conjugate of the disclosure has the following structure:

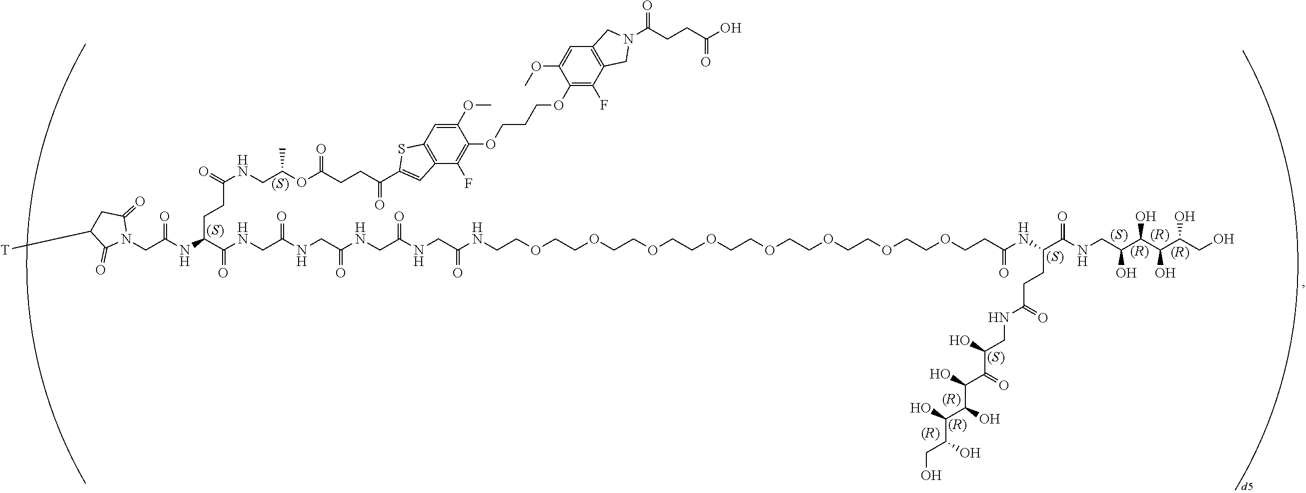

wherein T is an anti-cMet antibody, for example having a heavy chain of the amino acid sequence as shown in SEQ ID NO: 4 and a light chain of the amino acid sequence as shown in SEQ ID NO: 5; or for example having a heavy chain of the amino acid sequence as shown in SEQ ID NO: 8 and a light chain of the amino acid sequence as shown in SEQ ID NO: 9; and d5 is an integer of 6-8, such as 7 or 8.

(SEQ ID NO: 4)
QVQLEQSGPEVVKPGASVKVSCKASGYSFTSYWMHWVRQAPGQGLEWMG
MIDPSDSESRLNQKFKDRVTMTVDTPTSTVYMELSSPRSEDTAVYYCAR
SGYHGTSYWYFDVWGQGLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYT
QKSLSLSPGK
(SEQ ID NO: 5)
DIVMTQSPLSLPVTPGEPASISCRSSKSLLHSDGITYLYWYLQKPGQSP
QLLIYQMSNLASGVPDRFSGSGSGTDFTLKISRVEAEDVGVYYCAQNLE
LPPTFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
(SEQ ID NO: 8)
QVQLVQSGAEVKKPGASVKVSCKASGYIFTAYTMHWVRQAPGQGLEWMG
WIKPNNGLANYAQKFQGRVTMTRDTSISTAYMELSRLRSDDTAVYYCAR
SEITTEFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK
(SEQ ID NO: 9)
DIVMTQSPDSLAVSLGERATINCKSSESVDSYANSFLHWYQQKPGQPPK
LLIYRASTRESGVPDRFSGSGSGTDFTLTISSLQAEDVAVYYCQQSKED
PLTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

In some embodiments, the conjugate of the disclosure has the following structure:

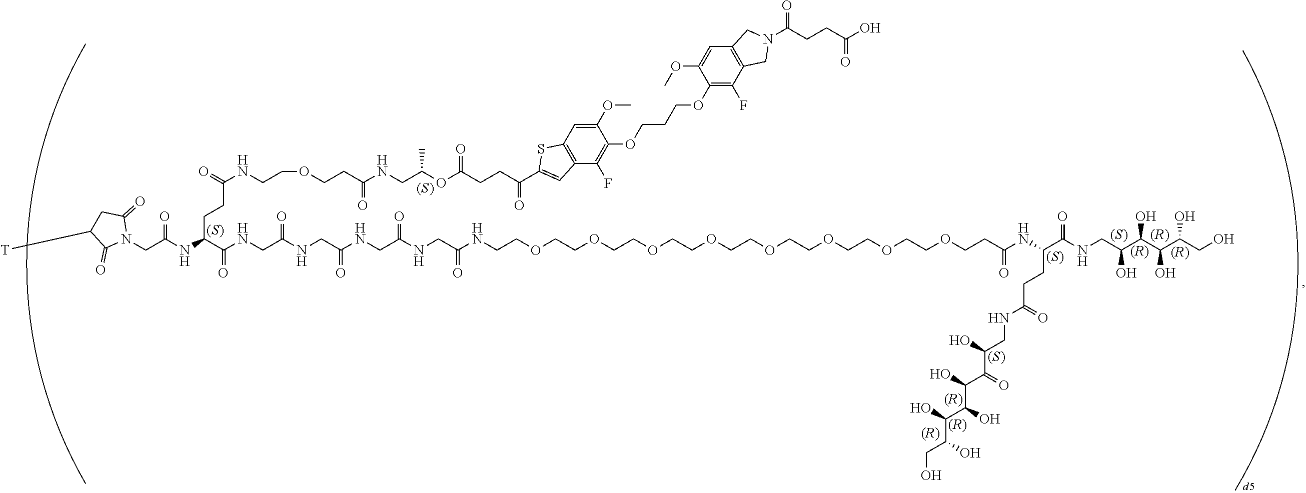

wherein T is an anti-cMet antibody, for example having a heavy chain of the amino acid sequence as shown in SEQ ID NO: 4 and a light chain of the amino acid sequence as shown in SEQ ID NO: 5; or for example having a heavy chain of the amino acid sequence as shown in SEQ ID NO: 8 and a light chain of the amino acid sequence as shown in SEQ ID NO: 9; and d5 is an integer of 6-8, such as 7 or 8.

In some embodiments, the conjugate of the disclosure has the following structure:

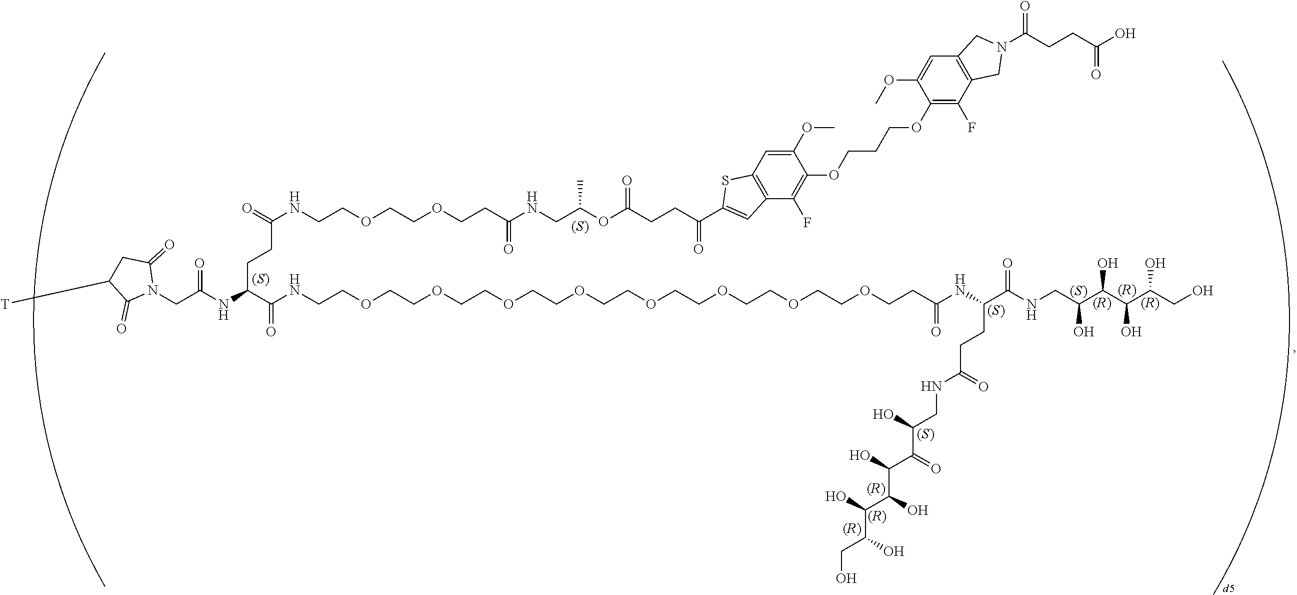

wherein T is an anti-cMet antibody, for example having a heavy chain of the amino acid sequence as shown in SEQ ID NO: 4 and a light chain of the amino acid sequence as shown in SEQ ID NO: 5; or for example having a heavy chain of the amino acid sequence as shown in SEQ ID NO: 8 and a light chain of the amino acid sequence as shown in SEQ ID NO: 9; and d5 is an integer of 6-8, such as 7 or 8.

In some embodiments, the conjugate of the disclosure has the following structure:

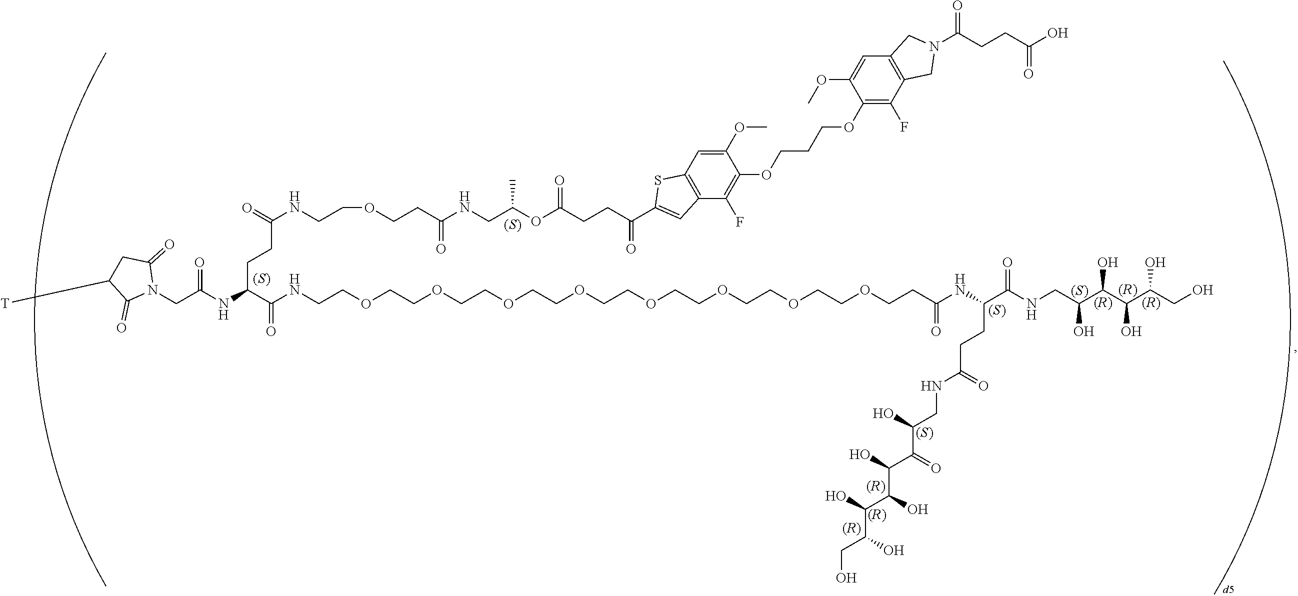

wherein T is an anti-cMet antibody, for example having a heavy chain of the amino acid sequence as shown in SEQ ID NO: 4 and a light chain of the amino acid sequence as shown in SEQ ID NO: 5; or for example having a heavy chain of the amino acid sequence as shown in SEQ ID NO: 8 and a light chain of the amino acid sequence as shown in SEQ ID NO: 9; and d5 is an integer of 6-8, such as 7 or 8.

In some embodiments, the conjugate of the disclosure has the following structure:

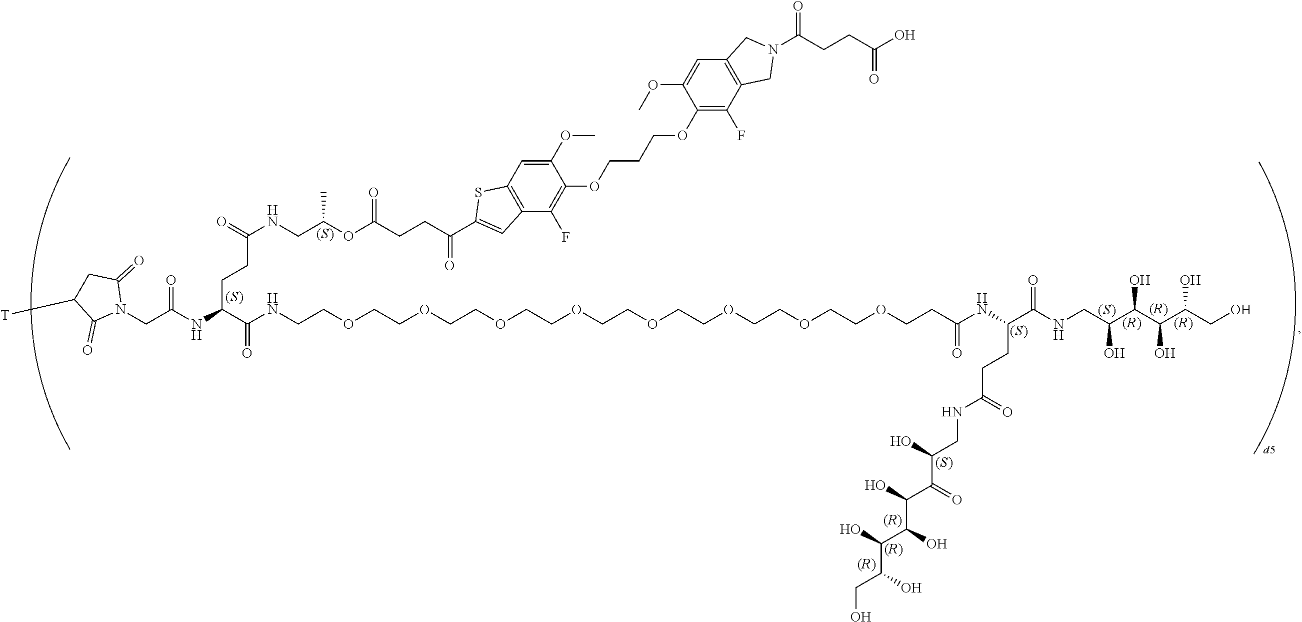

wherein T is an anti-cMet antibody, for example having a heavy chain of the amino acid sequence as shown in SEQ ID NO: 4 and a light chain of the amino acid sequence as shown in SEQ ID NO: 5; or for example having a heavy chain of the amino acid sequence as shown in SEQ ID NO: 8 and a light chain of the amino acid sequence as shown in SEQ ID NO: 9; and d5 is an integer of 6-8, such as 7 or 8.

In some embodiments, the conjugate of the disclosure has the following structure:

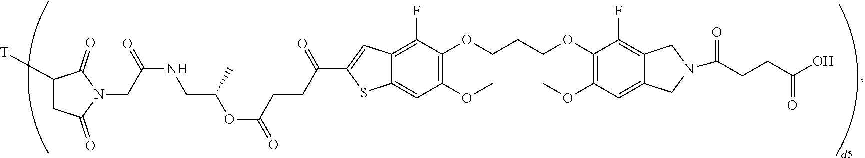

wherein T is an anti-cMet antibody, for example having a heavy chain of the amino acid sequence as shown in SEQ ID NO: 4 and a light chain of the amino acid sequence as shown in SEQ ID NO: 5; or for example having a heavy chain of the amino acid sequence as shown in SEQ ID NO: 8 and a light chain of the amino acid sequence as shown in SEQ ID NO: 9; and d5 is an integer of 6-8, such as 7 or 8.

In some embodiments, the conjugate of the disclosure has the following structure:

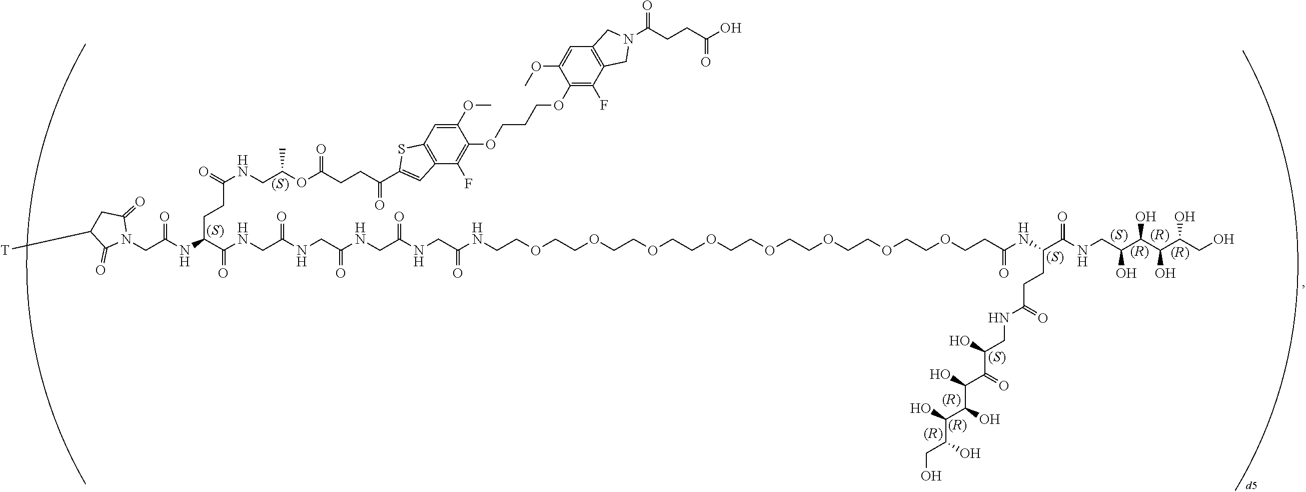

wherein T is an anti-mesothelin antibody, for example having a heavy chain of the amino acid sequence as shown in SEQ ID NO: 6 and a light chain of the amino acid sequence as shown in SEQ ID NO. d5 is an integer of 6-8, such as 7 or 8.

(SEQ ID NO: 6)
MEFGLSWVFLVAILKGVQCEVQLVESGGGLVQPGGSLRLSCAASGFDFS
RYWMSWVRQAPGKGLEWIGEINPDSSTIVYTPSLKDKFIISRDNAKNTL
YLQMNSLRAEDTALYYCARRGSHYYGYRTGYFDVWGAGTTVTVSSASTK
GPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKS
RWQQGNVFSCSVMHEALHNHYTQKSLSLSPGK
(SEQ ID NO: 7)
MVSPLQFLRLLLLWVPASRGDVVMTQSPAFLSVTPGEKVTMTCSASSSV
SYMYWHQQKPDQAPKLLIYDTSNLASGVPVRFSGSGSGTDFTFTISRME
AEDAATYYCQQWSSYPPTFGGGTKVEIKRTVAAPSVFIFPPSDEQLKSG
TASVVCLLNNFYPREAKVQWKVDNALQSGNSQESVTEQDSKDSTYSLSS
TLTLSKADYEKHKVYACEVTHQGLSSPVTKSFNRGEC

In some embodiments, the conjugate of the disclosure has the following structure:

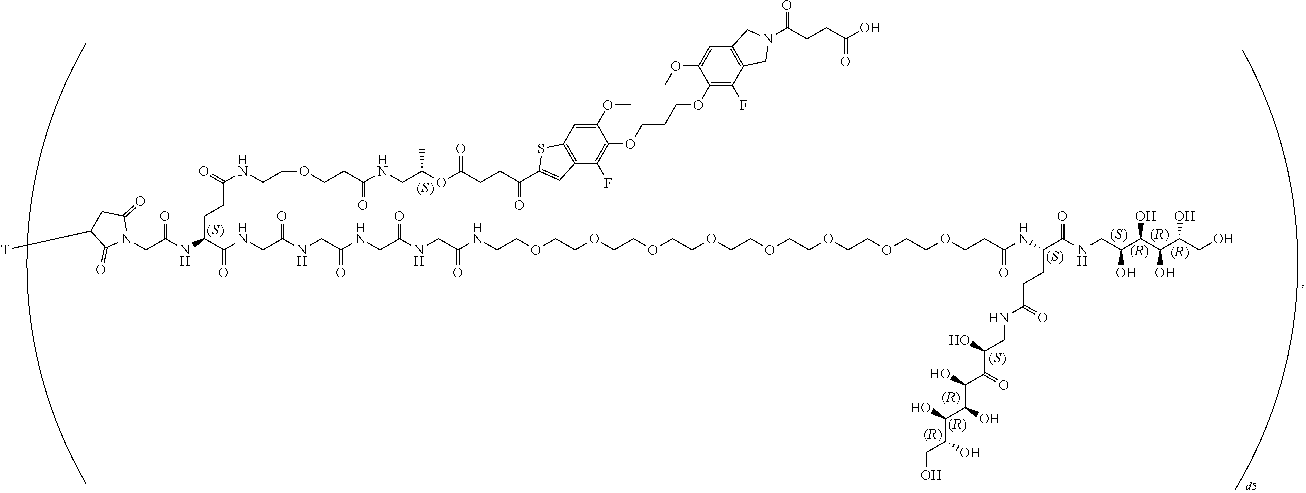

wherein T is an anti-mesothelin antibody, for example having a heavy chain of the amino acid sequence as shown in SEQ ID NO: 6 and a light chain of the amino acid sequence as shown in SEQ ID NO: 7, and d5 is an integer of 6-8, such as 7 or 8.

In some embodiments, the conjugate of the disclosure has the following structure:

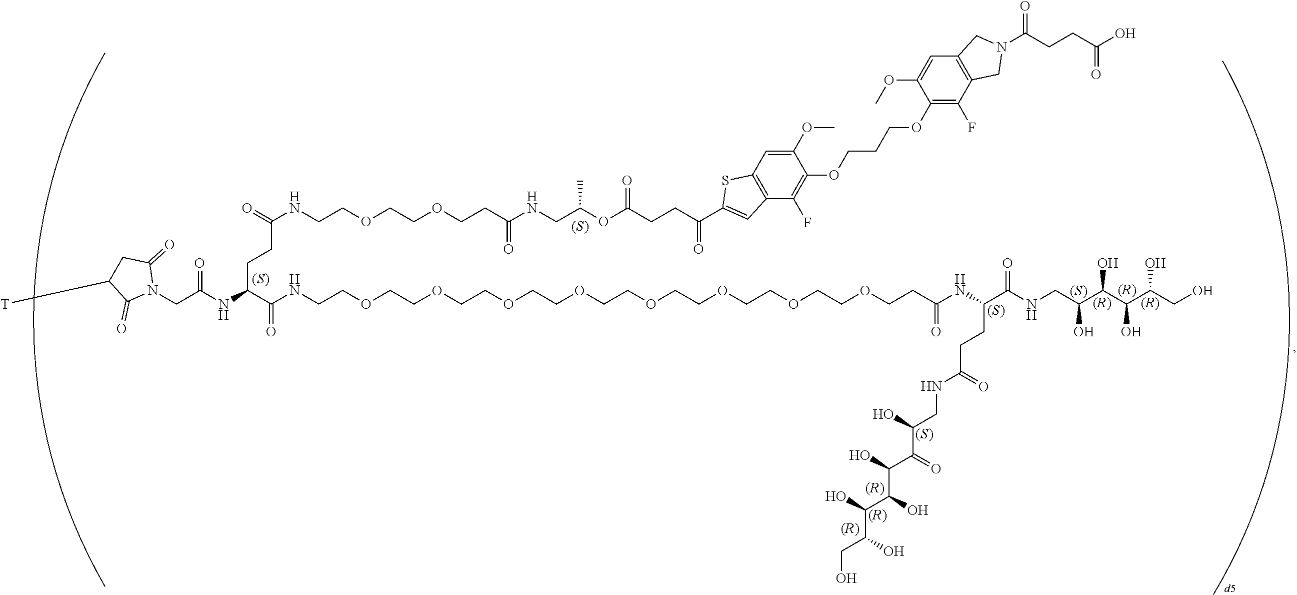

wherein T is an anti-mesothelin antibody, for example having a heavy chain of the amino acid sequence as shown in SEQ ID NO: 6 and a light chain of the amino acid sequence as shown in SEQ ID NO: 7, and d5 is an integer of 6-8, such as 7 or 8.

In some embodiments, the conjugate of the disclosure has the following structure:

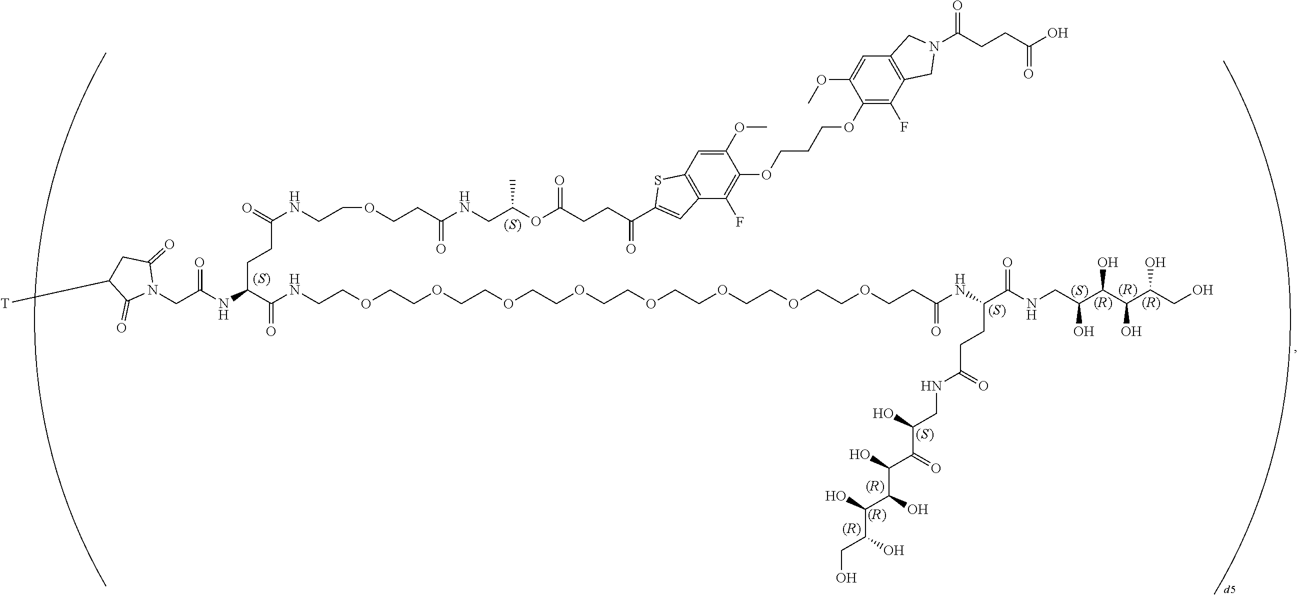

wherein T is an anti-mesothelin antibody, for example having a heavy chain of the amino acid sequence as shown in SEQ ID NO: 6 and a light chain of the amino acid sequence as shown in SEQ ID NO: 7, and d5 is an integer of 6-8, such as 7 or 8.

In some embodiments, the conjugate of the disclosure has the following structure:

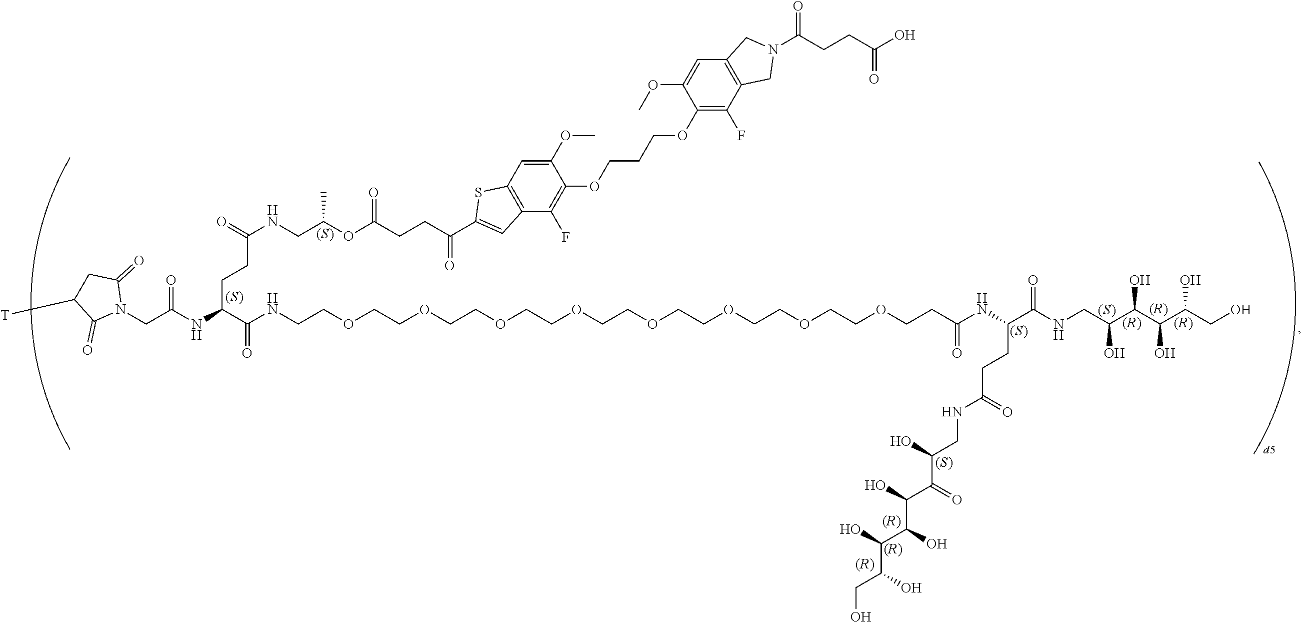

wherein T is an anti-mesothelin antibody, for example having a heavy chain of the amino acid sequence as shown in SEQ ID NO: 6 and a light chain of the amino acid sequence as shown in SEQ ID NO: 7, and d5 is an integer of 6-8, such as 7 or 8.

In some embodiments, the conjugate of the disclosure has the following structure:

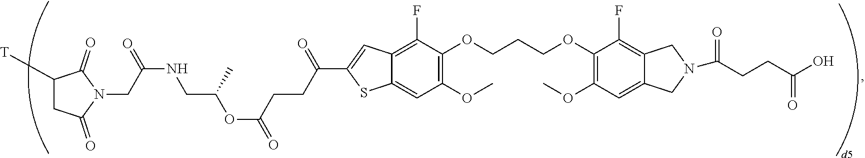

wherein T is an anti-mesothelin antibody, for example having a heavy chain of the amino acid sequence as shown in SEQ ID NO: 6 and a light chain of the amino acid sequence as shown in SEQ ID NO: 7, and d5 is an integer of 6-8, such as 7 or 8.

In some embodiments, the conjugate of the disclosure has the following structure:

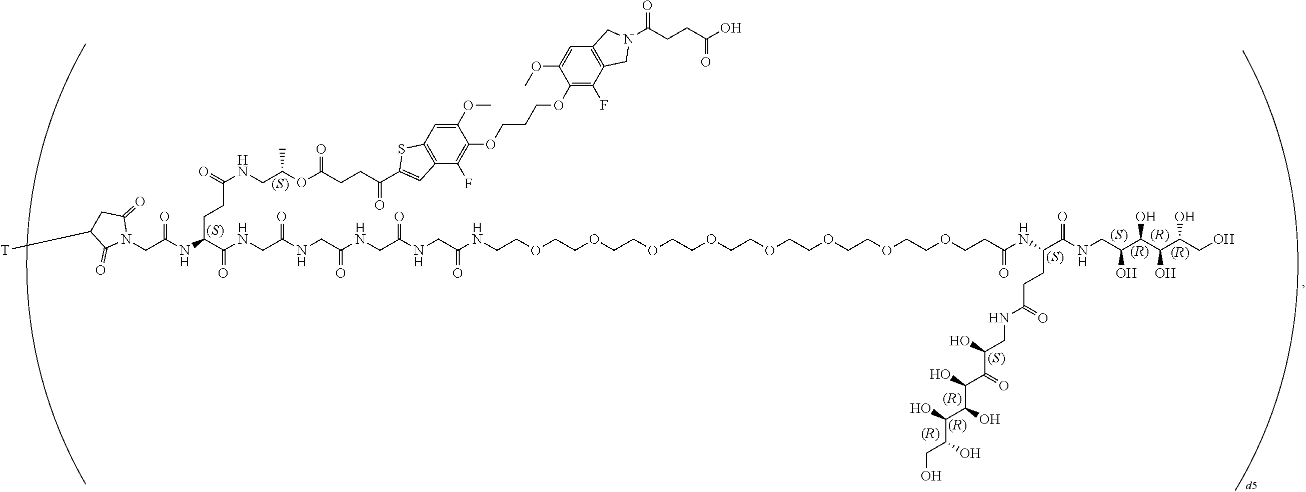

wherein T is an anti-Claudin18.2 antibody, and d5 is an integer of 6-8, such as 7 or 8.

In some embodiments, the conjugate of the disclosure has the following structure:

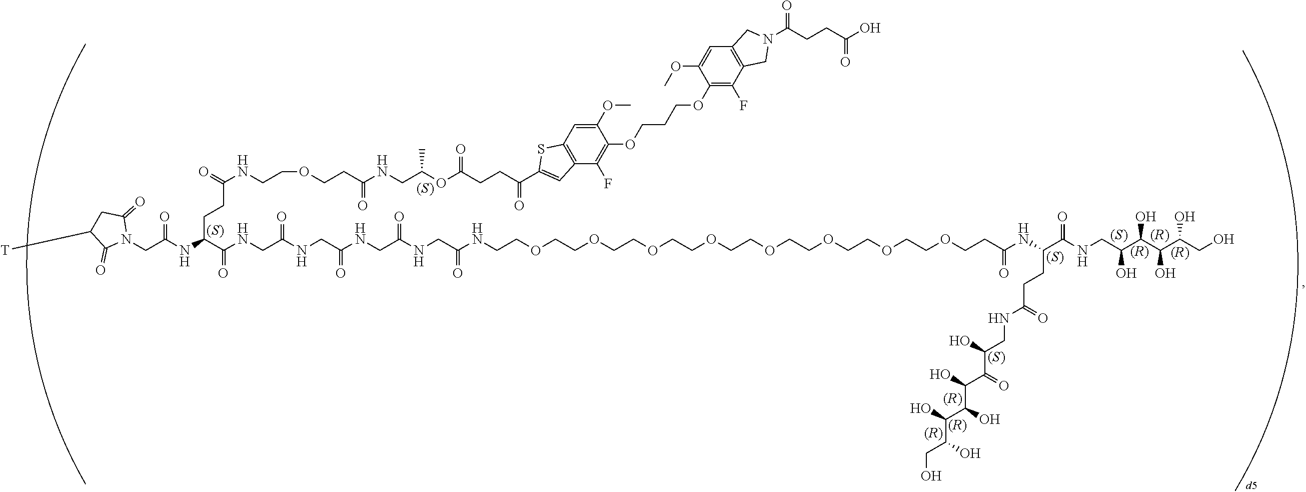

wherein T is an anti-Claudin18.2 antibody, and d5 is an integer of 6-8, such as 7 or 8. PP25C

In some embodiments, the conjugate of the disclosure has the following structure:

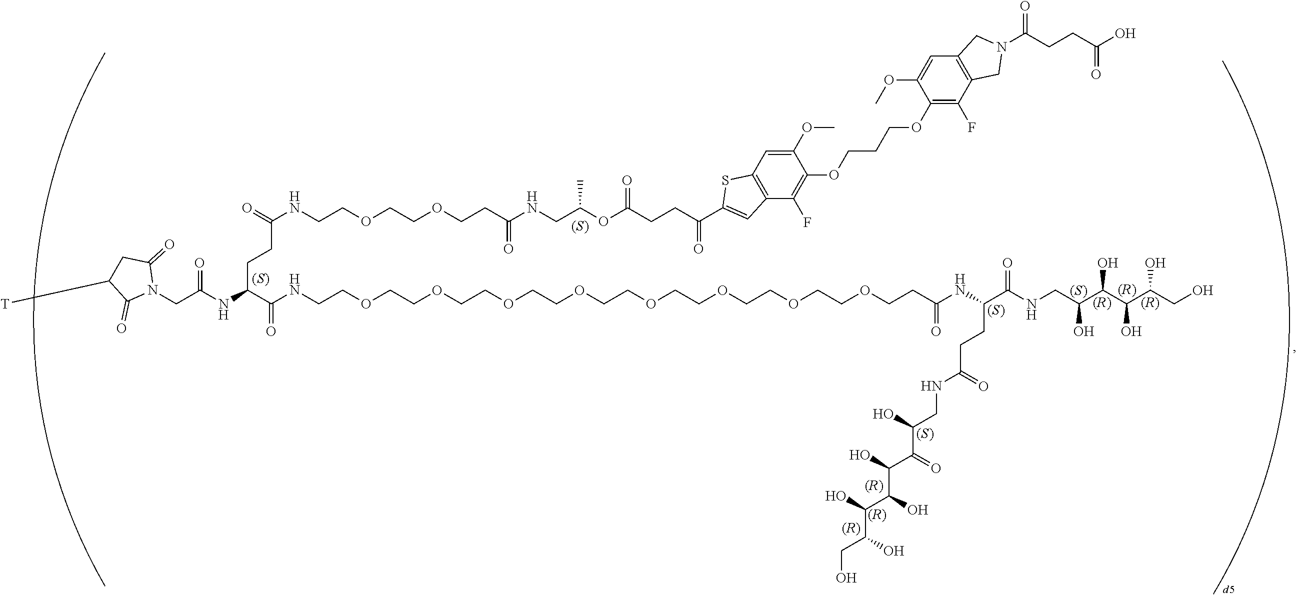

wherein T is an anti-Claudin18.2 antibody, and d5 is an integer of 6-8, such as 7 or 8.

In some embodiments, the conjugate of the disclosure has the following structure:

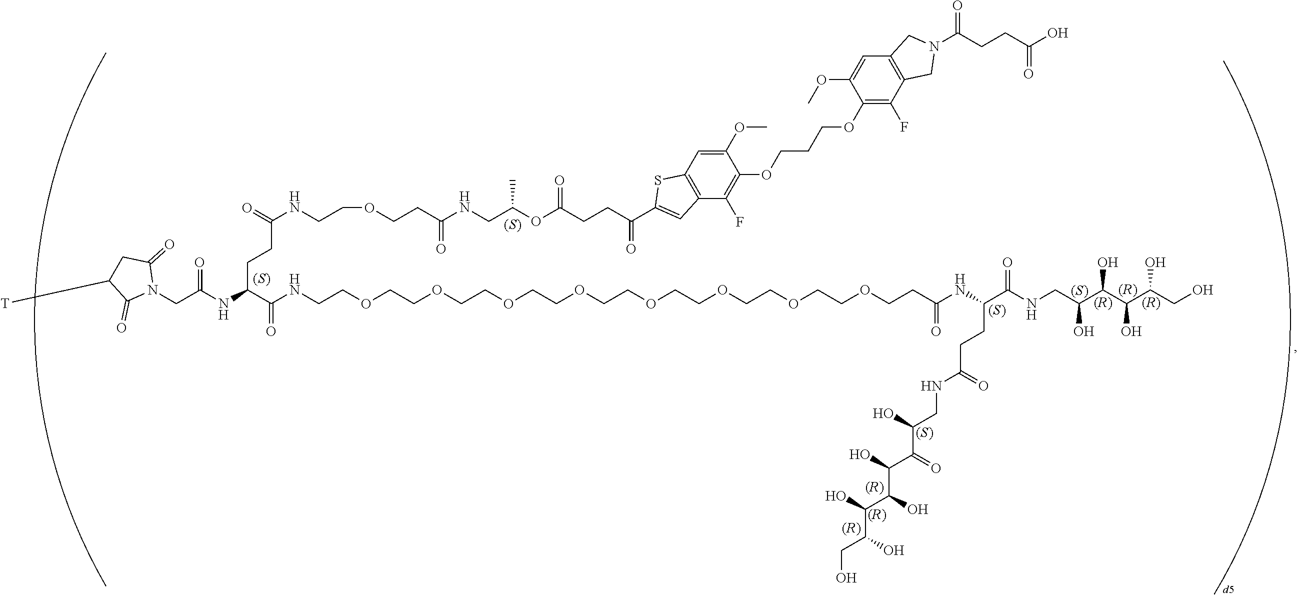

wherein T is an anti-Claudin18.2 antibody, and d5 is an integer of 6-8, such as 7 or 8.

In some embodiments, the conjugate of the disclosure has the following structure:

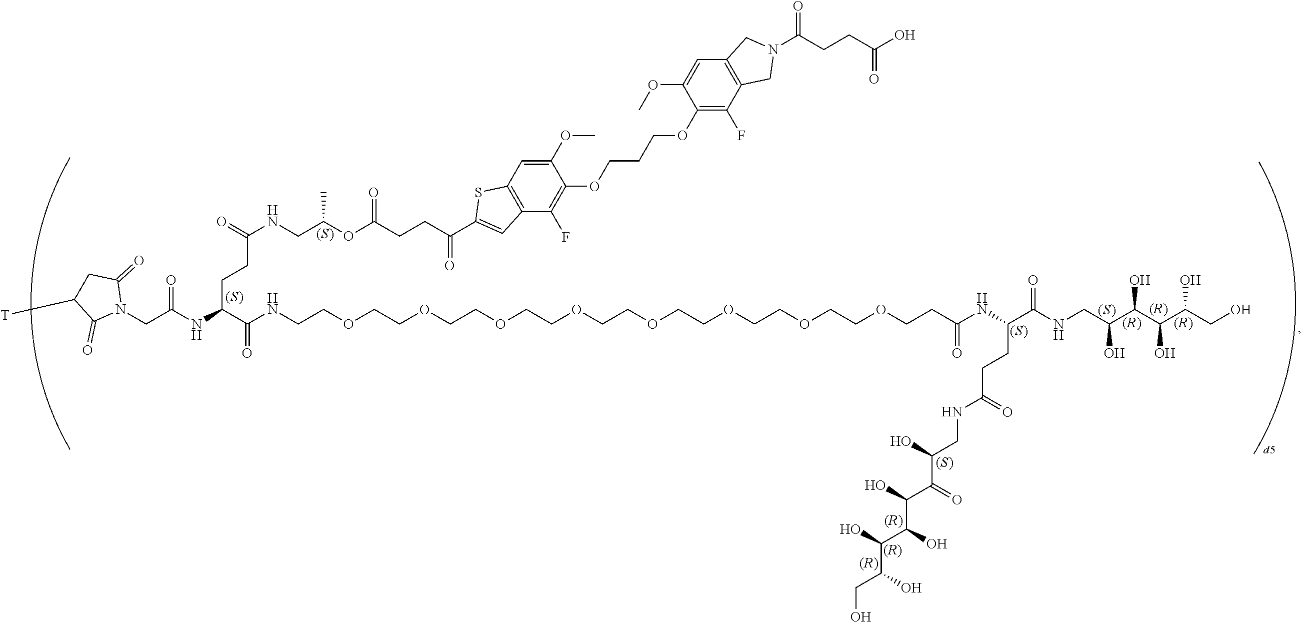

wherein T is an anti-Claudin18.2 antibody, and d5 is an integer of 6-8, such as 7 or 8.

In some embodiments, the conjugate of the disclosure has the following structure:

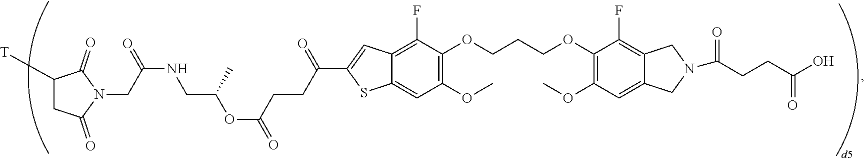

wherein T is an anti-Claudin18.2 antibody, and d5 is an integer of 6-8, such as 7 or 8.

### Conjugates

In some embodiments, conjugates of the disclosure comprise one or more occurrences of D, wherein D is a STING agonist, wherein the one or more occurrences of D may be the same or different.

In some embodiments, one or more occurrences of T is attached to the Linker- STING agonist moiety, wherein the one or more occurrences of T may be the same or different. In some embodiments, one or more Linker- STING agonist moieties that comprises one or more occurrences of D are connected to one T (e.g., an antibody).

In some embodiments, the conjugate of the disclosure comprise a T that has a molecular weight of about kDa or greater (e.g., about 60 kDa or greater; about 80 kDa or greater; about 100 kDa or greater; about 120 kDa or greater; about 140 kDa or greater; about 160 kDa or greater; about 180 kDa or greater; or about 200 kDa or greater, or about 40-200 kDa, about 40-180 kDa, about 40-140 kDa, about 60-200 kDa, about 60-180 kDa, about 60-140 kDa, about 80-200 kDa, about 80-180 kDa, about 80-140 kDa, about 100-200 kDa, about 100-180 kDa, or about 100-140 kDa) and has a sulfhydryl (i.e., —SH or thiol) group.

In some embodiments, the total number of sulfide bonds formed between the Linker-STING agonist moieties and the T (or total number of attachment points) is 10 or less (e.g., 8, 6, 4, or 2).

In some embodiments, for conjugation with one or more Linker-STING agonist moieties, the T has a molecular weight of about 40 kDa or greater (e.g., about 60 kDa or greater, about 80 kDa or greater, about 100 kDa or greater, about 120 kDa or greater, about 140 kDa or greater, about 160 kDa or greater, or about 180 kDa or greater; or about 40-200 kDa, about 40-180 kDa, about 40-140 kDa, about 60-200 kDa, about 60-180 kDa, about 60-140 kDa, about 80-200 kDa, about 80-180 kDa, about 80-140 kDa, about 100-200 kDa, about 100-180 kDa, or about 100-140 kDa).

In some embodiments, for conjugation with one or more Linker-STING agonist moieties, the T has a molecular weight of about 40 kDa to about 200 kDa. In some embodiments, for conjugation with one or more Linker-STING agonist moieties, the T has a molecular weight of about 40 kDa to about 80 kDa.

In some embodiments, for conjugation with one or more Linker-STING agonist moieties, the T has a molecular weight of 40 kDa to 200 kDa. In some embodiments, for conjugation with one or more Linker-STING agonist moieties, the T has a molecular weight of 40 kDa to 80 kDa.

In some embodiments, Ts in this molecular weight range include, but are not limited to, for example, antibody fragments, such as, for example, Fabs.

In some embodiments, for conjugation with one or more Linker-STING agonist moieties, the T has a molecular weight of about 60 kDa to about 120 kDa.

In some embodiments, for conjugation with one or more Linker-STING agonist moieties, the T has a molecular weight of 60 kDa to 120 kDa.

In some embodiments, Ts in this molecular weight range include, but are not limited to, for example, camelids, Fab2, scFvFc, and the like.

In some embodiments, for conjugation with one or more Linker-STING agonist moieties, the T has a molecular weight of about 140 kDa to about 180 kDa.

In some embodiments, for conjugation with one or more Linker-STING agonist moieties, the T has a molecular weight of 140 kDa to 180 kDa.

In some embodiments, Ts in this molecular weight range include, but are not limited to, for example, full length antibodies, such as, IgG, IgM.

In some embodiments, the targeting ligands, the linkers and the drug or prodrug fragments described herein can be assembled into the conjugate or scaffold of the disclosure, for example according to the disclosed techniques and methods. Therapeutic and targeting conjugates of the disclosure, and methods for producing them, are described below by way of non-limiting example.

In some embodiments, the total number of sulfide bonds formed between the Linker-STING agonist moiety and the T (or total number of attachment points) is 8 or less.

In some embodiments, the total number of sulfide bonds formed between the Linker-STING agonist moiety and the T (or total number of attachment points) is 8. In some embodiments, the total number of sulfide bonds formed between the Linker-STING agonist moiety and the T (or total number of attachment points) is 6.

In some embodiments, the total number of sulfide bonds formed between the Linker-STING agonist moiety and the T (or total number of attachment points) is 5. In some embodiments, the total number of sulfide bonds formed between the Linker-STING agonist moiety and the T (or total number of attachment points) is 4. In some embodiments, the total number of sulfide bonds formed between the Linker-STING agonist moiety and the T (or total number of attachment points) is 3. In some embodiments, the total number of sulfide bonds formed between the Linker-STING agonist moiety and the T (or total number of attachment points) is 2.

In some embodiments, the ratio between Linker-STING agonist moiety and the T is between about 1:1 and about 8:1. In some embodiments, the ratio between Linker-STING agonist moiety and the T is between about 1:1 and about 6:1. In some embodiments, the ratio between Linker-STING agonist moiety and the T is between about 1:1 and about 4:1. In some embodiments, the ratio between Linker-STING agonist moiety and the T is between about 2:1 and about 2:1.

In some embodiments, the ratio between Linker-STING agonist moiety and the T is between about 6:1 and about 8:1.

In some embodiments, the ratio between Linker-STING agonist moiety and the T is about 8:1.

In some embodiments, the ratio between Linker-STING agonist moiety and the T is about 6:1.

In some embodiments, the disclosure also relates to a Linker-STING agonist moiety comprising at least two moieties, wherein each moiety is capable of conjugation to a thiol group in a T so as to form a protein-Linker-Drug conjugate.

In some embodiments, one or more thiol groups of a T are produced by reducing a protein. The one or more thiol groups of the T may then react with one or more Linker-STING agonist moieties that are capable of conjugation to a thiol group from the T with the Linker-STING agonist moiety. In some embodiments, the at least two moieties connected to the T are maleimide groups.

In some embodiments, the antibodies may be activated for conjugation with Linker-STING agonist moiety by treatment with a reducing agent such as DTT (Cleland's reagent, dithiothreitol) or TCEP (tris(2-carboxyethyl)phosphine hydrochloride). In some embodiments, full length, monoclonal antibodies can be reduced with an excess of TCEP to reduce disulfide bonds (e.g., between the cysteine present in the corresponding parent antibodies) to yield a reduced form of the antibody. The newly introduced and unpaired cysteine may remain available for reaction with Linker-STING agonist moiety to form the antibody conjugates of the present disclosure. In some embodiments, an excess of Linker-STING agonist moiety is added to effect conjugation and form the antibody-drug conjugate, and the conjugation mixture is purified to remove excess Linker-drug intermediate and other impurities.

In some embodiments, for conjugating of the Linker-STING agonist moiety, a T has a molecular weight of 40 kDa or greater (e.g., 60 kDa or greater; 80 kDa or greater; or 100 kDa or greater; 120 kDa or greater; 140 kDa or greater; 160 kDa or greater or 180 kDa or greater). In some embodiments, the ratio of T per Linker-STING agonist moiety is between about 1:1 and about 1:8; about 1:1 and about 1:6; between about 1:1 and about 1:5; between about 1:1 and about 1:4; between about 1:1 and about 1:3; or between about 1:1 and about 1:2.

Ts in this molecular weight range include, but are not limited to, for example, full length antibodies, such as, IgG, IgM.

In some embodiments, for conjugation with one or more Linker-STING agonist moieties a T has a molecular weight of 60 kDa to 120 kDa. In some embodiments, the ratio of T per Linker-STING agonist moiety is about 1:1 and about 1:8; between about 1:1 and about 1:6; between about 1:1 and about 1:5; between about 1:1 and about 1:4; between about 1:1 and about 1:3; or between about 1:1 and about 1:2.

Ts in this molecular weight range include, but are not limited to, for example, antibody fragments such as, for example Fab2, scFcFv and camelids.

In some embodiments, for conjugation with one or more Linker-STING agonist moieties a T has a molecular weight of 40 kDa to 80 kDa. In some embodiments, the ratio of T per Linker-STING agonist moiety is about 1:1 and about 1:8; between about 1:1 and about 1:6; between about 1:1 and about 1:5; between 1:1 and about 1:4; between about 1:1 and about 1:3, or between about 1:1 and about 1:2.

In some embodiments, Ts in this molecular weight range include, but are not limited to, for example, antibody fragments, such as, Fabs.

In some embodiments, the disclosure features a scaffold useful to conjugate with either or both of a protein-based recognition-molecule (T) and a STING agonist moiety (D).

In some embodiments, the drug-carrying compound-linker constructs (i.e., without linking to a T), described herein each typically have a polydispersity index (PDI) of 1.

Conjugates and compound-linker constructs disclosed herein can be purified (i.e., removal of any starting materials) by extensive diafiltration. If necessary, additional purification by size exclusion chromatography can be conducted to remove any aggregated conjugates. In general, the conjugates as purified typically contain less than 5% (e.g., <2% w/w) aggregated conjugates as determined by SEC; less than 0.5% (e.g., <0.1% w/w) free (unconjugated) drug as determined by RP-HPLC; less than 1% drug carrying-peptide-containing compound-linker constructs as determined by SEC and less than 2% (e.g., <1% w/w) unconjugated T as determined by HIC-HPLC.

In some embodiments, the targeting moiety T comprises an antibody, an antibody fragment, a nucleic acid based molecule, a carbohydrate, a peptide, or a modified peptide, in particular an antibody or an antigen-binding fragment, which is designed to target the Human Epidermal Growth Factor Receptor (EGFR), a plasminogen activator, a cytotoxic T-lymphocyte associated antigen (CTLA) such as CTLA-4, PD-1, PD-L1, KIR, TIM3, VISTA, TIGIT, LAG3, OX40, RORI, ROR2, vascular endothelial growth factor (VEGF), fibroblast growth factor receptor (FGFR), platelet-derived growth factor (PDGF), transforming growth factor (TGF), neurotrophic factors, a nerve growth factor, platelet-derived growth factor (PDGF), interleukin receptors, transforming growth factor (TGF), estrogen receptor, progesterone receptor, c-Kit, cMET, ErbB2/Her2, ErbB3/Her3, ErbB4/Her4, CD3, CD20, CD22, CD30, CD33, CD40, CD47, CD79, CD123, CD133, CD166, CD137, the mesothelin protein, EpCAM, FLT3, PSMA, PSCA, STEAP, CEA, folate receptor, the CD39/CD73 receptors, adenosine receptors, SLC34A2 gene product, the EphA2 tyrosine kinase, the MucI/Muc16 cell-surface antigens, ALK, AFP, bcr-Abl, PAP.

In some embodiments, the targeting moiety T is trastuzumab.

In some aspects, the present disclosure provides a pharmaceutical composition comprising a therapeutically effective amount of the compound-linker construct or the conjugate of the present disclosure and at least one pharmaceutically acceptable excipient.

In some embodiments, the weight ratio of the compound-linker construct or the conjugate of the present disclosure to the excipient is within the range from about 0.0001 to about 10.

In some embodiments, the pharmaceutical composition further comprising at least one additional active agents selected from STING agonist compounds, anti-viral compounds, antigens, adjuvants, CTLA-4 and PD-1 pathway antagonists and other immunomodulatory agents, lipids, liposomes, peptides, anti-cancer agents, and chemotherapeutic agents.

In some aspects, the present disclosure provides a use of the compound-linker construct or the conjugate of the present disclosure, and/or the pharmaceutical composition described herein for the manufacture of a medicament.

In some embodiments, wherein the medicament is used for treating or preventing a disease or disorder in a subject in need thereof.

In some embodiments, wherein the medicament is used for treating a STING-mediated disease or disorder in a subject.

In some embodiments, wherein the medicament is used for treating a cancer in a subject in need thereof.

In some embodiments, wherein the medicament is used for inducing an immune response in a subject.

In some embodiments, wherein the medicament is used for inducing STING-dependent type I interferon production in a subject.

In some embodiments, wherein the medicament is used for inducing a STING-dependent cytokine production in a subject.

In some embodiments, wherein the medicament is used for treating a cell proliferation disorder in a subject.

In some embodiments, wherein the cell proliferation disorder is cancer.

In some aspects, the present disclosure provides the compound-linker construct or the conjugate of the present disclosure, and/or the pharmaceutical composition described herein, for use in therapy.

In some aspects, the present disclosure provides the compound-linker construct or the conjugate of the present disclosure, and/or the pharmaceutical composition described herein, for use in treating or preventing a disease or disorder in a subject in need thereof.

In some aspects, the present disclosure provides the compound-linker construct or the conjugate of the present disclosure, and/or the pharmaceutical composition described herein, for use in treating a STING-mediated disease or disorder in a subject.

In some aspects, the present disclosure provides the compound-linker construct or the conjugate of the present disclosure, and/or the pharmaceutical composition described herein, for use in treatinga cancer in a subject in need thereof.

In some aspects, the present disclosure provides the compound-linker construct or the conjugate of the present disclosure, and/or the pharmaceutical composition described herein, for use in inducing an immune response in a subject.

In some aspects, the present disclosure provides the compound-linker construct or the conjugate of the present disclosure, and/or the pharmaceutical composition described herein, for use in inducing STING-dependent type I interferon production in a subject.

In some aspects, the present disclosure provides the compound-linker construct or the conjugate of the present disclosure, and/or the pharmaceutical composition described herein, for use in inducing a STING-dependent cytokine production in a subject.

In some aspects, the present disclosure provides the compound-linker construct or the conjugate of the present disclosure, and/or the pharmaceutical composition described herein, for use in treating a cell proliferation disorder in a subject.

In some embodiments, wherein the cell proliferation disorder is cancer.

In some aspects, the present disclosure provides the compound-linker construct or the conjugate of the present disclosure, and/or the pharmaceutical composition described herein, for use as a STING agonist.

In some aspects, the present disclosure provides the compound-linker construct or the conjugate of the present disclosure, and/or the pharmaceutical composition described herein, for use as a medicament.

In some aspects, the present disclosure provides a method of treating or preventing a disease or disorder in a subject in need thereof, comprising administering to the subject a therapeutically effective amount of a conjugate disclosed herein.

In some embodiments, the present disclosure provides a method of treating a disease or disorder in a subject in need thereof, comprising administering to the subject a therapeutically effective amount of a conjugate disclosed herein.

In some embodiments, the present disclosure relates to a method of treating a cancer in a subject in need thereof, comprising administering to the subject an effective amount of a conjugate disclosed herein.

In some aspects, the present disclosure provides a method of inducing an immune response in a subject, said method comprising administering a therapeutically effective amount of the compound-linker construct or the conjugate of the present disclosure, and/or the pharmaceutical composition described herein to the subject.

In some aspects, the present disclosure provides a method of inducing STING-dependent type I interferon production in a subject, said method comprising administering a therapeutically effective amount of the compound-linker construct or the conjugate of the present disclosure, and/or the pharmaceutical composition described herein to the subject.

In some aspects, the present disclosure provides a method of inducing a STING-dependent cytokine production in a subject, said method comprising administering a therapeutically effective amount of the compound-linker construct or the conjugate of the present disclosure, and/or the pharmaceutical composition described herein to the subject.

In some aspects, the present disclosure provides a method of treating a cell proliferation disorder in a subject, said method comprising administering a therapeutically effective amount of the compound-linker construct or the conjugate of the present disclosure, and/or the pharmaceutical composition defined herein to the subject.

In some embodiments, wherein the cell proliferation disorder is cancer, cancer metastasis, cardiovascular disease, an immunological disorder, fibrosis, or an ocular disorder.

In some embodiments, the conjugate disclosed herein is administered to the subject.

In some embodiments, the present disclosure provides a method of treating or preventing a disease or disorder in a subject in need thereof, comprising administering to the subject an efficient amount of at least one conjugate of the disclosure; wherein said conjugate releases one or more therapeutic agent upon biodegradation.

In some embodiments, the present disclosure provides a method of treating a disease or disorder in a subject in need thereof, comprising administering to the subject an efficient amount of at least one conjugate of the disclosure; wherein said conjugate releases one or more therapeutic agent upon biodegradation.

In some embodiments, the present disclosure the conjugate is an antibody-STING agonist conjugate. In some embodiments, the disease or disorder is cancer. These conjugates are potentially useful in treating diseases or disorders including, but not limited to, cell proliferation disorders. Cell-proliferation disorders include, but are not limited to, cancers, benign papillomatosis, gestational trophoblastic diseases, and benign neoplastic diseases, such as skin papilloma (warts) and genital papilloma.

In some embodiments, the disclosure provides methods of treatment or prevention of STING mediated diseases and disorders. Exemplary diseases/disorders include, but are not limited to, cancer, infectious disease (e.g., HIV, HBV, HCV, HPV, and influenza), and vaccine adjuvant.

In specific embodiments, the disease or disorder to be treated is a cell proliferation disorder. In certain embodiments, the cell proliferation disorder is cancer. In particular embodiments, the cancer is selected from brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, malignant mesothelioma, sarcomas, lymphomas, glandular cancers, thyroid cancers, heart tumors, germ cell tumors, malignant neuroendocrine (carcinoid) tumors, midline tract cancers, and cancers of unknown primary (i.e., cancers in which a metastasized cancer is found but the original cancer site is not known). In particular embodiments, the cancer is present in an adult patient; in additional embodiments, the cancer is present in a pediatric patient. In particular embodiments, the cancer is AIDS-related.

In specific embodiments, the cancer is selected from brain and spinal cancers. In particular embodiments, the cancer is selected from the group consisting of anaplastic astrocytomas, glioblastomas, astrocytomas, and estheosioneuroblastomas (also known as olfactory blastomas). In particular embodiments, the brain cancer is selected from the group consisting of astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astorcytoma/malignant glioma, visual pathway and hypothalmic glioma, and primary central nervous system lymphoma. In specific instances of these embodiments, the brain cancer is selected from the group consisting of glioma, glioblastoma multiforme, paraganglioma, and suprantentorial primordial neuroectodermal tumors (sPET).

In specific embodiments, the cancer is selected from cancers of the head and neck, including nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers of the larynx (e.g., laryngeal squamous cell carcinomas, rhabdomyosarcomas), and cancers of the eye or ocular cancers. In particular embodiments, the ocular cancer is selected from the group consisting of intraocular melanoma and retinoblastoma.

In specific embodiments, the cancer is selected from leukemia and cancers of the blood. In particular embodiments, the cancer is selected from the group consisting of myeloproliferative neoplasms, myelodysplastic syndromes, myelodysplastic/myeloproliferative neoplasms, acute myeloid leukemia (AML), myelodysplastic syndrome (MDS), chronic myelogenous leukemia (CML), myeloproliferative neoplasm (MPN), post-MPN AML, post- MDS AML, del(5q)-associated high risk MDS or AML, blast-phase chronic myelogenous leukemia, angioimmunoblastic lymphoma, acute lymphoblastic leukemia, Langerans cell histiocytosis, hairy cell leukemia, and plasma cell neoplasms including plasmacytomas and multiple myelomas. Leukemias referenced herein may be acute or chronic.

In specific embodiments, the cancer is selected from skin cancers. In particular embodiments, the skin cancer is selected from the group consisting of melanoma, squamous cell cancers, and basal cell cancers.

In specific embodiments, the cancer is selected from cancers of the reproductive system. In particular embodiments, the cancer is selected from the group consisting of breast cancers, cervical cancers, vaginal cancers, ovarian cancers, prostate cancers, penile cancers, and testicular cancers. In specific instances of these embodiments, the cancer is a breast cancer selected from the group consisting of ductal carcinomas and phyllodes tumors. In specific instances of these embodiments, the breast cancer may be male breast cancer or female breast cancer. In specific instances of these embodiments, the cancer is a cervical cancer selected from the group consisting of squamous cell carcinomas and adenocarcinomas. In specific instances of these embodiments, the cancer is an ovarian cancer selected from the group consisting of epithelial cancers.

In specific embodiments, the cancer is selected from cancers of the gastrointestinal system. In particular embodiments, the cancer is selected from the group consisting of esophageal cancers, gastric cancers (also known as stomach cancers), gastrointestinal carcinoid tumors, pancreatic cancers, gallbladder cancers, colorectal cancers, and anal cancer. In instances of these embodiments, the cancer is selected from the group consisting of esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gallbladder adenocarcinomas, colorectal adenocarcinomas, and anal squamous cell carcinomas.

In specific embodiments, the cancer is selected from liver and bile duct cancers. In particular embodiments, the cancer is liver cancer (also known as hepatocellular carcinoma). In particular embodiments, the cancer is bile duct cancer (also known as cholangiocarcinoma); in instances of these embodiments, the bile duct cancer is selected from the group consisting of intrahepatic cholangiocarcinoma and extrahepatic cholangiocarcinoma.

In specific embodiments, the cancer is selected from kidney and bladder cancers. In particular embodiments, the cancer is a kidney cancer selected from the group consisting of renal cell cancer, Wilms tumors, and transitional cell cancers. In particular embodiments, the cancer is a bladder cancer selected from the group consisting of urethelial carcinoma (a transitional cell carcinoma), squamous cell carcinomas, and adenocarcinomas.

In specific embodiments, the cancer is selected from bone cancers. In particular embodiments, the bone cancer is selected from the group consisting of osteosarcoma, malignant fibrous histiocytoma of bone, Ewing sarcoma, chordoma (cancer of the bone along the spine).

In specific embodiments, the cancer is selected from lung cancers. In particular embodiments, the lung cancer is selected from the group consisting of non-small cell lung cancer, small cell lung cancers, bronchial tumors, and pleuropulmonary blastomas.

In specific embodiments, the cancer is selected from malignant mesothelioma. In particular embodiments, the cancer is selected from the group consisting of epithelial mesothelioma and sarcomatoids.

In specific embodiments, the cancer is selected from sarcomas. In particular embodiments, the sarcoma is selected from the group consisting of central chondrosarcoma, central and periosteal chondroma, fibrosarcoma, clear cell sarcoma of tendon sheaths, and Kaposi's sarcoma.

In specific embodiments, the cancer is selected from lymphomas. In particular embodiments, the cancer is selected from the group consisting of Hodgkin lymphoma (e.g., Reed-Sternberg cells), non-Hodgkin lymphoma (e.g., diffuse large B-cell lymphoma, follicular lymphoma, mycosis fungoides, Sezary syndrome, primary central nervous system lymphoma), cutaneous T-cell lymphomas, primary central nervous system lymphomas.

In specific embodiments, the cancer is selected from glandular cancers. In particular embodiments, the cancer is selected from the group consisting of adrenocortical cancer (also known as adrenocortical carcinoma or adrenal cortical carcinoma), pheochromocytomas, paragangliomas, pituitary tumors, thymoma, and thymic carcinomas.

In specific embodiments, the cancer is selected from thyroid cancers. In particular embodiments, the thyroid cancer is selected from the group consisting of medullary thyroid carcinomas, papillary thyroid carcinomas, and follicular thyroid carcinomas.

In specific embodiments, the cancer is selected from germ cell tumors. In particular embodiments, the cancer is selected from the group consisting of malignant extracranial germ cell tumors and malignant extragonadal germ cell tumors. In specific instances of these embodiments, the malignant extragonadal germ cell tumors are selected from the group consisting of nonseminomas and seminomas.

In specific embodiments, the cancer is selected from heart tumors. In particular embodiments, the heart tumor is selected from the group consisting of malignant teratoma, lymphoma, rhabdomyosacroma, angiosarcoma, chondrosarcoma, infantile fibrosarcoma, and synovial sarcoma.

In specific embodiments, the cell-proliferation disorder is selected from benign papillomatosis, benign neoplastic diseases and gestational trophoblastic diseases. In particular embodiments, the benign neoplastic disease is selected from skin papilloma (warts) and genital papilloma. In particular embodiments, the gestational trophoblastic disease is selected from the group consisting of hydatidiform moles, and gestational trophoblastic neoplasia (e.g., invasive moles, choriocarcinomas, placental -site trophoblastic tumors, and epithelioid trophoblastic tumors).

In some embodiments, the disease or disorder is a neurodegenerative disease. Exemplary neurodegenerative diseases include, but are not limited to, multiple sclerosis, Huntington's disease, Alzheimer's disease, Parkinson's disease, amyotrophic lateral sclerosis (ALS). The scope of the diseases would be readily recognized by a skilled artisan in the field.

In some embodiments, the disease or disorder is mediated by the activity of STING.

As used herein, the terms “treatment” and “treating” refer to all processes in which there may be a slowing, interrupting, arresting, controlling, or stopping of the progression of a disease or disorder described herein. The terms do not necessarily indicate a total elimination of all disease or disorder symptoms.

The terms “administration of and or “administering” a compound should be understood to include providing a compound described herein, or a pharmaceutically acceptable salt thereof, and compositions or conjugates of the foregoing to a subject.

The amount of a compound, a conjugate, or a pharmaceutical composition administered to a subject is an amount sufficient to induce an immune response and/or to induce STING-dependent type I interferon production in the subject. In an embodiment, the amount of a compound, a conjugate, or a pharmaceutical composition can be an “effective amount” or “therapeutically effective amount,” such that the subject compound is administered in an amount that will elicit, respectively, a biological or medical (i.e., intended to treat) response of a tissue, system, animal, or human that is being sought by a researcher, veterinarian, medical doctor, or other clinician. An effective amount does not necessarily include considerations of toxicity and safety related to the administration of a compound, a conjugate, or a pharmaceutical composition.

An effective amount of a compound or a conjugate will vary with the particular compound chosen (e.g., considering the potency, efficacy, and/or half-life of the compound or conjugate); the route of administration chosen; the condition being treated; the severity of the condition being treated; the age, size, weight, and physical condition of the subject being treated; the medical history of the subject being treated; the duration of the treatment; the nature of a concurrent therapy; the desired therapeutic effect; and like factors and can be routinely determined by the skilled artisan.

The term “subject” (alternatively referred to herein as “patient”) as used herein refers to an animal, preferably a mammal, most preferably a human, who has been the object of treatment, observation or experiment.

As used herein, the term “immune response” relates to any one or more of the following: specific immune response, non-specific immune response, both specific and nonspecific response, innate response, primary immune response, adaptive immunity, secondary immune response, memory immune response, immune cell activation, immune cell proliferation, immune cell differentiation, and cytokine expression. In certain embodiments, a compound of general formula (Y-1), (Y-2), (Y-3), (A), (B), (C), I, II, III, IV or V, or a pharmaceutically acceptable salt of the foregoing, or a conjugate thereof, is administered in conjunction with one or more additional therapeutic agents including anti -viral compounds, vaccines intended to stimulate an immune response to one or more predetermined antigens, adjuvants, CTLA-4 and PD-1 pathway antagonists and other immunomodulatory agents, lipids, liposomes, peptides, anti-cancer agents, and chemotherapeutic agents, etc.

As used herein, the term “compound-linker construct” refers to a construct comprises a com-pound of formula (Y-1), (Y-2), (Y-3), (A), (B), (C), I, II, III, IV or V as defined herein and a linker L¹ as defined herein, which are covalently bonded to each other as explained in detail above.

As used herein, the term conjugate refers to a conjugate comprising a compound of formula (Y-1), (Y-2), (Y-3), (A), (B), (C), I, II, III, IV or V, a linker L¹, and a targeting moiety T, wherein the linker L¹ links the compound of formula (Y-1), (Y-2), (Y-3), (A), (B), (C), I, II, III, IV or V to the targeting moiety T by covalent bonds. The targeting moiety may have from 1 to 30 compounds of formula (Y-1), (Y-2), (Y-3), (A), (B), (C), I, II, III, IV or V attached via a linker L¹, wherein the linker L¹ may in each case be identical, or different.

As used herein, the term “linker” can refer to a short, flexible, rigid, cleavable, non-cleavable, hydrophilic or hydrophobic chain covalently connecting the compound of formula (Y-1), (Y-2), (Y-3), (A), (B), (C), I, II, III, IV or V with the targeting moiety T. A cleavable linker can be cleaved by enzymes such as proteases. A cleavable linker can be a valine-citrulline linker or a valine-alanine linker.

As used herein, the term “targeting moiety” refers to moiety that has targeting capabilities such that it may specifically target a specific antigen, in particular a tumor antigen. Targeting in this context means that the moiety specifically binds to or is immunologically reactive toward the specific antigen. Preferred antigens include proteins, preferably proteins that can only be found in or on tumor cells. Suitable targeting moieties include antibodies, antibody fragments, nucleic acid based molecules, carbohydrates, peptides or modified peptides. A preferred targeting moiety according to the disclosure is an antibody or an antibody fragment. A preferred conjugates according to the disclosure are so-called antibody-drug conjugates (ADCs). A preferred conjugates according to the disclosure are so-called immunostimulatory antibody-drug conjugates (iADCs).

The targeting moiety may direct the compound of formula (Y-1), (Y-2), (Y-3), (A), (B), (C), I, II, III, IV or V of the conjugates specifically to tumor cells, in order to deliver the compound of formula (Y-1), (Y-2), (Y-3), (A), (B), (C), I, II, III, IV or V in a cell-specific manner. The principle is described in Polakis, P., Pharmacol.

Revs., 2016, 68, 3-19. If the linker between the targeting moiety and the compound of formula (Y-1), (Y-2), (Y-3), (A), (B), (C), I, II, III, IV or V is designed to be cleavable, the compound of formula (Y-1), (Y-2), (Y-3), (A), (B), (C), I, II, III, IV or V will diffuse into the cell and contact the STING protein.

As used herein, the term “antibody drug conjugate” (“ADC”) refers to conjugate as defined herein, wherein the targeting moiety T is an antibody, antibody fragment, a protein, a peptide, or a peptide mimic.

As used herein, the term “immunostimulatory antibody drug conjugate” (“iADC”) refers to conjugate as defined herein, wherein the targeting moiety T is an antibody, antibody fragment, a protein, a peptide, or a peptide mimic, and the payload is an immunostimulatory compound.

As used herein, the term “antibody” can refer to an immunoglobulin molecule that specifically binds to, or is immunologically reactive toward, a specific antigen. Antibodies can include, for example, polyclonal, monoclonal, genetically engineered antibodies, and antigen binding ragements thereof. An antibody can be for example, murine, chimeric, humanized, heteroconjugate, bispecific, diabody, triabody, or tetrabody. The antigen binding fragment can include, for example, Fab′, F(ab′)₂, Fab, Fv, rIgG, and scFv.

The term “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

The term “antibody that binds to the same epitope” as a reference antibody as used herein, refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “monoclonal antibody” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “epitope” refers to the particular site on an antigen molecule to which an antibody binds.

As used herein, a “tumor antigen” can be an antigenic substance associated with a tumor or cancer call, and can trigger an immune response in a host.

An antigen can elicit an immune response. An antigen can be a protein, polysaccharide, lipid, or glycolipid, which can be recognized by an immune cell, such as a T cell or a B cell. Exposure of immune cells to one or more of these antigens can elicit a rapid cell division and differentia-tion response resulting in the formation of clones of the exposed T cells and B cells. B cells can differentiate into plasma cells which in turn can produce antibodies which selectively bind to the antigens.

In cancer, there are four general groups of tumor antigens: (i) viral tumor antigens which can be identical for any viral tumor of this type, (ii) carcinogenic tumor antigens which can be spe-cific for patients and for the tumors, (iii) isoantigens of the transplantation type or tumorspecific transplantation antigens which can be different in all individual types of tumors but can be the same in different tumors caused by the same virus; and (iv) embryonic antigens.

As a result of the discovery of tumor antigens, tumor antigens have become important in the development of new cancer treatments that can specifically target the cancer. This has led to the development of antibodies directed against these tumor antigens.

In addition to the development of antibodies against tumor antigens for cancer treatment, antibodies that target immune cells to boost the immune response have also been developed. For example, an anti-CD40 antibody that is a CD40 agonist can be used to activate dendritic cells to enhance the immune response.

The term “T” refers to a molecule that recognizes and binds to a cell surface marker or receptor such as, a transmembrane protein, surface immobilized protein, or proteoglycan. In some embodiments, the T comprises an engineered cysteine. Examples of T include but are not limited to, antibodies, peptides, lipocalins, proteins, peptides or peptide mimics, and the like. The T, in addition to targeting the conjugate to a specific cell, tissue or location, may also have certain therapeutic effect such as antiproliferative (cytostatic and/or cytotoxic) activity against a target cell or pathway. The T comprises or may be engineered to comprise at least one chemically reactive group such as, —COOH, primary amine, secondary amine —NHR, —SH, or a chemically reactive amino acid moiety or side chains such as, for example, tyrosine, histidine, cysteine, or lysine. In some embodiments, a T may be a ligand (LG) or targeting moiety which specifically binds or complexes with a cell surface molecule, such as a cell surface receptor or antigen, for a given target cell population. Following specific binding or complexing of the ligand with its receptor, the cell is permissive for uptake of the ligand or ligand-drug-conjugate, which is then internalized into the cell. As used herein, a ligand that “specifically binds or complexes with” or “targets” a cell surface molecule preferentially associates with a cell surface molecule via intermolecular forces. In some embodiments, the ligand can preferentially associate with the cell surface molecule with a Kd of less than about 50 nM, less than about 5 nM, or less than 500 μM. Techniques for measuring binding affinity of a ligand to a cell surface molecule are well-known; for example, one suitable technique, is termed surface plasmon resonance (SPR). In some embodiments, the ligand is used for targeting and has no detectable therapeutic effect as separate from the drug which it delivers. In some embodiments, the ligand functions both as a targeting moiety and as a therapeutic or immunomodulatory agent (e.g., to enhance the activity of the active drug or prodrug). The term “PEG unit” ss used herein refers to a polyethylene glycol subunit having the formula

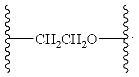

In some embodiments, the PEG unit comprises multiple PEG subunits.

The term “STING agonist”, as used herein, refers to a compound or moiety which is capable of interacting with STING, e.g., by binding to STING and/or inducing downstream signal transduction (e.g., characterized by activation of the molecules associated with STING function). This includes direct phosphorylation of STING, IRF3 and/or NF-κB and could also include STAT6. In some embodiments, STING pathway activation results in increased production of type 1 interferons (mainly IFN-α and IFN-b) and/or expression of interferon-stimulated genes.

The term “STING agonist moiety”, as used herein, refers to a moiety derived from a STING agonist and capable of interacting with STING. In some embodiments, the STING agonist moiety is a moiety derived from a STING agonist to allow the moiety being linked to the rest of a conjugate of the present disclosure.

The conjugates of the disclosure are useful in methods for treating or ameliorating a viral infection, disease, a syndrome, a condition or a disorder that is affected by the agonism of STING. Such methods comprise, consist of and/or consist essentially of administering to a subject, including an animal, a mammal, and a human in need of such treatment, amelioration and/or prevention, a therapeutically effective amount of a conjugate of the disclosure, or an enantiomer, diastereomer, solvate or pharmaceutically acceptable salt thereof.

In some embodiments, conjugates of the disclosure, or an enantiomer, diastereomer, solvate or pharmaceutically acceptable salt form thereof are useful for treating or ameliorating diseases, syndromes, conditions, or disorders such as melanoma, colon cancer, breast cancer, prostate cancer, lung cancer, fibrosarcoma, and hepatitis B.

The terms “conjugate(s) of the disclosure” or “conjugate(s) of the present disclosure”, as used herein, mean a conjugate as defined herein, in any form, i.e., any tautomeric form, any isomeric form, any salt or non-salt form (e.g., as a free acid or base form, or as a salt, particularly a pharmaceutically acceptable salt thereof) and any physical form thereof (e.g., including non-solid forms (e.g., liquid or semi-solid forms), and solid forms (e.g., amorphous or crystalline forms, specific polymorphic forms, solvate forms, including hydrate forms (e.g., mono-, di- and hemi-hydrates)), and mixtures of various forms.

Accordingly, included within the present disclosure are the conjugates as disclosure herein, in any salt or non-salt form and any physical form thereof, and mixtures of various forms. While such are included within the present disclosure, it will be understood that the conjugates of the present disclosure, in any salt or non-salt form, and in any physical form thereof, may have varying levels of activity, different bioavailabilities and different handling properties for formulation purposes.

The term “halogen”, as used herein, unless otherwise indicated, means fluoro, chloro, bromo or iodo. The preferred halogen groups include F, Cl and Br. The terms “haloC₁₋₆alkyl”, “haloC₂₋₆alkenyl”, “haloC₂_-alkynyl” and “haloC₁₋₆alkoxy” mean a C₁₋₆alkyl, C₂₋₆alkenyl, C₂₋₆alkynyl or C₁₋₆alkoxy in which one or more (in particular, 1, 2 or 3) hydrogen atoms have been replaced by halogen atoms, especially fluorine or chlorine atoms. In some embodiment, preferred are fluoroC₁₋₆alkyl, fluoroC₂₋₆alkenyl, fluoroC₂₋₆alkynyl and fluoroC₁₋₆alkoxy groups, in particular fluoroC₁₋₃alkyl, for example, CF₃, CHF₂, CH₂F, CH₂CH₂F, CH₂CHF₂, CH₂CF₃ and fluoroC₁₁3alkoxy groups, for example, OCF₃, OCHF₂, OCH₂F, OCH₂CH₂F, OCH₂CHF₂ or OCH₂CF₃, and most especially CF₃, OCF₃ and OCHF₂.

As used herein, unless otherwise indicated, alkyl includes saturated monovalent hydrocarbon radicals having straight, branched or cyclic moieties. For example, alkyl radicals include methyl, ethyl, propyl, isopropyl, cyclcopropyl, n-butyl, isobutyl, sec-butyl, t-butyl, cyclcobutyl, n-pentyl, 3- (2-methyl) butyl, 2-pentyl, 2-methylbutyl, neopentyl, cyclcopentyl, n- hexyl, 2-hexyl, 2-methylpentyl and cyclohexyl. Similary, C₁_s, as in C₁_salkyl is defined to identify the group as having 1, 2, 3, 4, 5, 6, 7 or 8 carbon atoms in a linear or branched arrangement.

Alkylene means a difunctional group obtained by removal of a hydrogen atom from an alkyl group that is defined above. For example, methylene (i.e., —CH₂—), ethylene (i.e., —CH₂—CH₂— or —CH(CH₃)—) and propylene (i.e., —CH₂—CH₂—CH₂—, —CH(—CH₂—CH₃)—or —CH₂—CH(CH₃)—).

As used herein, the term “alkenyl” refers to a monovalent straight or branched chain, unsaturated aliphatic hydrocarbon radical having a number of carbon atoms in the specified range and including one or more double bonds.

As used herein, the term “alkenylene” refers to a bivalent straight chain, unsaturated aliphatic hydrocarbon radical having a number of carbon atoms in the specified range and including one or more double bonds.

As used herein, the term “alkynyl” refers to a monovalent straight or branched chain, unsaturated aliphatic hydrocarbon radical having a number of carbon atoms in the specified range and including one or more triple bonds.

As used herein, the term “alkynylene” refers to a bivalent straight chain, unsaturated aliphatic hydrocarbon radical having a number of carbon atoms in the specified range and including one or more triple bonds.

As used herein, the term “alkoxy” as used herein, alone or in combination, includes an alkyl group connected to the oxy connecting atom. The term “alkoxy” also includes alkyl ether groups, where the term ‘alkyl’ is defined above, and ‘ether’ means two alkyl groups with an oxygen atom between them. Examples of suitable alkoxy groups include methoxy, ethoxy, n-propoxy, i-propoxy, n-butoxy, s-butoxy, t-butoxy, methoxymethane (also referred to as ‘dimethyl ether’), and methoxy ethane (also referred to as ‘ethyl methyl ether’).

The term “aryl”, as used herein, unless otherwise indicated, by itself or as part of another substituent refers to a monocyclic or polycyclic aromatic hydrocarbon. Phenyl and naphthyl are preferred aryls. The most preferred aryl is phenyl.

The term “heterocyclic”, “heterocyclyl”, or “heterocyclic”, as used herein, unless otherwise indicated, by itself or as part of another substituent refers to unsubstituted and substituted mono- or polycyclic non-aromatic, partially unsaturated or fully saturated ring system containing one or more heteroatoms. Preferred heteroatoms include N, O, and S, including N-oxides, sulfur oxides, and dioxides. Preferably the ring is three to eight membered and is either fully saturated or has one or more degrees of unsaturation. Multiple degrees of substitution, preferably one, two or three, are included within the present definition.

Examples of such heterocyclic groups include, but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinyl sulfoxide, thiamorpholinyl sulfone and oxadiazolyl.

The term “heteroaryl”, as used herein, unless otherwise indicated, by itself or as part of another substituent refers to an aromatic ring system containing carbon(s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 10 hetero atoms. A polycyclic heteroaryl ring may contain fused, spiro or bridged ring junction, for example, bycyclic heteroaryl is a polycyclic heteroaryl. Bicyclic heteroaryl rings may contain from 8 to 12 member atoms. Monocyclic heteroaryl rings may contain from 5 to 8 member atoms (cabons and heteroatoms). Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyl adeninyl, quinolinyl or isoquinolinyl.

The term “carbocyclic” refers to a substituted or unsubstituted monocyclic ring, bicyclic ring, bridged ring, fused ring, sipiro ring non-aromatic ring system onle containing carbon atoms. Examplary “carbocyclic” groups includes but not limited to, cyclopropyl, cyclobutyl, cyclopentyl, cyclohexyl and so on.

The term “cycloalkyl” as used herein, unless otherwise indicated, by itself or as part of another substituent refers to a substituted or unsubstituted monocyclic, bicyclic or polycyclic non-aromatic saturated or partially unsatureated hydrocarbon group, which optionally includes an alkylene linker through which the cycloalkyl may be attached. Examplary “cycloalkyl” groups includes but not limited to, cyclopropyl, cyclobutyl, cyclopentyl, cyclohexyl and so on.

The term “carbonyl”, “—C═O”, “C═O”, “—CO”, “—C(O) “, and “CO” refer to the group

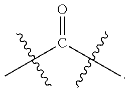 [SMILES: *C(*)=O]

The term “oxo” refers to the radical ═O.

Whenever the term “alkyl” or “aryl” or either of their prefix roots appear in a name of a substituent (e.g., aralky or dialkylamino), unless otherwise indicated, by itself or as part of another substituent, it shall be interpreted as including those limitations given above for “alkyl” and “aryl”. Designated numbers of carbon atoms (e.g., C₁₋₆) shall refer independently to the number of carbon atoms in an alkyl moiety or to the alkyl portion of a larger substituent in which alkyl appears as its prefix root.

As used herein, the term “fused ring” refers to a cyclic group formed by substituents on separate atoms in a straight or branched alkane, or to a cyclic group formed by substituents on separate atoms in another ring.

As used herein, the term “spirocycle” or “spirocyclic ring” refers to a pendant cyclic group formed by substituents on a single atom.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of ±1%, 2%, 3%, 4%, 5%, 10%, 15%, and ±20% and their numerical equivalents.

As used herein, the term “one or more” item includes a single item selected from the list as well as mixtures of two or more items selected from the list.

The term “optionally substituted”, as used herein, indicates that a group (such as an alkyl, cycloalkyl, alkoxy, heterocycloalkyl, aryl, or heteroaryl group) or ring or moiety may be unsubstituted, or the group, ring or moiety may be substituted with one or more substituent(s). In the case where groups may be selected from a number of alternative groups, the selected groups may be the same or different. Suitable substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “independently”, as used herein, means that where more than one substituent is selected from a number of possible substituents, those substituents may be the same or different.

Wherein the term “substituted” refers to a group mentioned above in which one or more (preferably 1-6, more preferably 1-3) hydrogen atoms are each independently replaced with the same or different substituent(s). Typical substituents include, but are not limited to, X, C₁₋₆alkyl, C₁₋₆alkoxy, C₃₋₂₀ cycloalkyl, —OR₁₃, SR₁₋₃, ═O, ═S, —C(O)R₁₃, —C(S)R₁₃, =NR₁₃, —C(O)OR₁₃, —C(S)OR₁₃, —NR₁₃R¹⁻⁴, —C(O)NR₁₃R¹⁻⁴, cyano, nitro, —S(O)₂R¹⁻³, —OS(O₂)OR₁₃, —OS(O)₂R¹⁻³, or —OP(O)(OR₁₋₃)(OR₁₋₄); wherein each X is independently a halogen (F, Cl, Br or I), and R¹³ and R¹⁴ is independently selected from —H, C₁₋₆ alkyl and C₁₋₆ haloalkyl. In some embodiments, the substituent(s) is independently selected from the group consisting of —F, —Cl, —Br, —I, —OH, trifluromethoxy, ethoxy, propyloxy, iso-propyloxy, n-butyloxy, isobutyloxy, t-butyloxy, —SCH₃, —SC₂H₅, formaldehyde group, —C(OCH₃), cyano, nitro, CF₃, —OCF₃, amino, dimethylamino, methyl thio, sulfonyl and acetyl. Particularly preferred substituent(s) is —F, —C₁ or —Br.

The substituents the two “R¹” of Formula (Y-1), (Y-2), (Y-3), (A), (B), (C), I, II, III, IV or V can be the same or different. Similar to “R¹”, and the two “R4”, “X1”, “X2”, or “X3” of Formula (Y-1), (Y-2), (Y-3), (A), (B), (C), I, II, III, IV or V can be the same or different.

It is intended that the definition of any substituent or variable at a particular location in a molecule be independent of its definitions elsewhere in that molecule. It is understood that substituents and substitution patterns on the compound as a STING agonist moiety [D] of this disclosure can be selected by one of ordinary skill in the art to provide compounds that are chemically stable and that can be readily synthesized by techniques know in the art as well as those methods set forth herein.

Compounds described herein, such as certain compounds of Formula (Y-1), (Y-2), (Y-3), (A), (B), (C), I, II, III, IV or V may contain asymmetrically substituted carbon atoms (or chiral centers) in the R or S configuration. The present disclosure includes racemic mixtures, relative and absolute stereoisomers, and mixtures of relative and absolute stereoisomers.

The compounds described herein, when specifically designated as the R—or S—isomer, either in a chemical name or in a drawing, should be understood as an enriched R-isomer or S-isomer, respectively. For example, in any of the embodiments described herein, such enriched R- or S-designated isomer can be substantially free (e.g., with less than 5%, less than 1%, or non-detectable, as determined by chiral HPLC) of the other isomer for the respective chiral center. The enriched R- or S-isomers can be prepared by methods exemplified in this disclosure, such as by using a chiral auxiliary such as R- or S-tert-butylsulfinamide in the synthetic process. Other methods for prepaing the enriched R- or S-isomers herein include, but are not limited to, chiral HPLC purifications of a stereoisomeric mixture, such as a racemic mixture. General methods for separating stereoisomers (such as enantiomers and/or diastereomers) using HPLC are known in the art.

Compounds described herein can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P ³⁵S, ¹⁸F ³⁶C₁, and¹²⁵1. Compounds that contain other isotopes of these and/or other atoms are within the scope of this disclosure. In some embodiments, one or more hydrogen atoms of any of the compounds described herein can be substituted with deuterium to provide the corresponding deterium-labeled or -enriched compounds.

The term “ring systems” as used herein, unless otherwise indicated, include but not limite to a carbocyclic ring, a heterocyclic ring, a heteroaromatic ring, etc., may also include only a heterocyclic ring, and/or a heteroaromatic ring, and the like, specifically includes which rings need to be determined according to the context, but anyway the “ring systems” do not include the cycloalkyl based on a C₁₋₆ alkyl or C₁₋₃ alkyl ogroup, and do not include the cycloalkoxy based on a C₁₋₆ alkoxy or C₁₋₃ alkoxy group.

Where the plural form (e.g., compounds, constructs, conjugates, salts) is used, this includes the singular (e.g., a single compound, a construct, a conjugate, a single salt). “A conjugate” does not exclude that (e.g., in a pharmaceutical formulation) more than one conjugate defined herein (or a salt thereof) is present, the “a” merely representing the indefinite article. “A” can thus preferably be read as “one or more”, less preferably alternatively as “one”.

The pharmaceutical compositions containing conjugates of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the conjugates into preparations that can be used pharmaceutically. Of course, the appropriate formulation is dependent upon the route of administration chosen.

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the conjugates of the present disclosure as the active ingredient as well as methods of preparing the instant compounds are also part of the present disclosure.

The conjugates of the present disclosure may also be present in the form of pharmaceutically acceptable salts. For use in medicine, the salts of the compounds of this disclosure refer to non-toxic “pharmaceutically acceptable salts”. The pharmaceutically acceptable salt forms include pharmaceutically acceptable acidic/anionic or basic/cationic salts. The pharmaceutically acceptable acidic/anionic salt generally takes a form in which the basic nitrogen is protonated with an inorganic or organic acid. Representative organic or inorganic acids include hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic. Pharmaceutically acceptable basic/cationic salts include, and are not limited to aluminum, calcium, chloroprocaine, choline, diethanolamine, ethylenediamine, lithium, magnesium, potassium, sodium and zinc.

The pharmaceutical compositions of the present disclosure comprise a conjugate (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy. Dosage unit form as used herein refers to physically discrete units suited as unitary dosages for the subject to be treated; each unit containing a predetermined quantity of conjugates can be calculated to produce the desired therapeutic effect in association with the required pharmaceutical carrier. The specification for the dosage unit forms of the disclosure is dictated by and directly dependent on the unique characteristics of the conjugates and the particular therapeutic effect to be achieved.

In practice, the conjugate, or a prodrug, or a metabolite, or pharmaceutically acceptable salts thereof, of this disclosure can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in- oil liquid emulsion. In addition to the common dosage forms set out above, the conjugate described herein or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this disclosure may include a pharmaceutically acceptable carrier, and a conjugate described herein or a pharmaceutically acceptable salt of the present disclosure, can also be included in pharmaceutical compositions in combination with one or more additional therapeutically active agents.

As modulators of the immune response, the conjugates of the present disclosure may also be used in monotherapy or in combination with another therapeutic agent in the treatment of diseases and conditions wherein modulation of STING is beneficial. Combination therapies according to the present disclosure thus comprise the administration of a conjugate of the present disclosure or a pharmaceutically acceptable salt thereof, and at least one other therapeutically active agent. In some embodiments, combination therapies according to the present disclosure comprise the administration of at least one conjugate of the present disclosure or a pharmaceutically acceptable salt thereof, and at least one other therapeutic agent. The conjugate(s) of the present disclosure and pharmaceutically acceptable salts thereof, and the other therapeutic agent(s) may be administered together in a single pharmaceutical composition or separately and, when administered separately this may occur simultaneously or sequentially in any order. The amounts of the conjugate(s) of the present disclosure and pharmaceutically acceptable salts thereof, and the other therapeutic agent(s) and the relative timings of administration will be selected in order to achieve the desired combined therapeutic effect. Thus, in a further aspect, there is provided a combination comprising a conjugate of the present disclosure or a pharmaceutically acceptable salt thereof, together with one or more other therapeutic agents.

The conjugate of the present disclosure and pharmaceutically acceptable salts thereof may be used in combination with radiotherapy and/or surgery and/or at least one other therapeutic agent which may be useful in the treatment of cancer and pre-cancerous syndromes. Any anti-neoplastic agent, anti-microtubule, anti-mitotic agent, hormone, hormonal analogues signal transduction pathway inhibitor, protein tyrosine kinase, or anti-angiogenic therapeutic agent, may be utilized in the combination. The scope of the other therapeutic agents would be readily recognized by a skilled artisan in the field.

The additional active agent(s) may be one or more agents selected from the group consisting of STING agonist compounds, anti-viral compounds, antigens, adjuvants, anti -cancer agents, CTLA-4, LAG-3 and PD-1 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, anti-tumor antibiotics, anti-metabolites, retinoids, and immunomodulatory agents including but not limited to anti -cancer vaccines. It will be understood that such additional active agent(s) may be provided as a pharmaceutically acceptable salt. It will be understood the descriptions of the above additional active agents may be overlapping. It will also be understood that the treatment combinations are subject to optimization, and it is understood that the best combination to use of the conjugate defined herein, or pharmaceutically acceptable salts of the foregoing, and one or more additional active agents will be determined based on the individual patient needs.

A conjugate disclosed herein may be used in combination with one or more other active agents, including but not limited to, other anti-cancer agents that are used in the prevention, treatment, control, amelioration, or reduction of risk of a particular disease or condition (e.g., cell proliferation disorders). In one embodiment, a conjugate disclosed herein is combined with one or more other anti -cancer agents for use in the prevention, treatment, control amelioration, or reduction of risk of a particular disease or condition for which the conjugates disclosed herein are useful. Such other active agents may be administered, by a route and in an amount commonly used therefor, contemporaneously or sequentially with a conjugate of the present disclosure.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this disclosure may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered conjugates moistened with an inert liquid diluent. Each tablet preferably contains from about 0.05 mg to about 5g of the active ingredient and each cachet or capsule preferably containing from about 0.05 mg to about 5g of the active ingredient. For example, a formulation intended for the oral administration to humans may contain from about 0.5 mg to about 5g of active agent, compounded with an appropriate and convenient amount of carrier material which may vary from about 5 to about 95 percent of the total composition. Unit dosage forms will generally contain between from about 1 mg to about 2g of the active ingredient, typically 25 mg, 50 mg, 100 mg, 200 mg, 300 mg, 400 mg, 500 mg, 600 mg, 800 mg, or 1000 mg.

Pharmaceutical compositions of the present disclosure suitable for parenteral administration may be prepared as solutions or suspensions of the active conjugates in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present disclosure can be in a form suitable for topical use such as, for example, an aerosol, cream, ointment, lotion, dusting powder, or the like. Further, the compositions can be in a form suitable for use in transdermal devices. These formulations may be prepared, utilizing a conjugate of this disclosure, or a pharmaceutically acceptable salt thereof, via conventional processing methods. As an example, a cream or ointment is prepared by admixing hydrophilic material and water, together with about 5 wt % to about lOwt % of the conjugate, to produce a cream or ointment having a desired consistency.

Pharmaceutical compositions of this disclosure can be in a form suitable for rectal administration and the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a conjugate of the present disclosure or pharmaceutically acceptable salts thereof, may also be prepared in powder or liquid concentrate form.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, inflammation, cancer, psoriasis, allergy/asthma, disease and conditions of the immune system, disease and conditions of the central nervous system (CNS), may be effectively treated by the administration of from about 0.01 to 50 mg of the conjugate per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

It is understood that the pharmaceutical compositions can be included in a container, pack, or dispenser together with instructions for administration.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The disclosure having now been described by way of written description, those of skill in the art will recognize that the disclosure can be practiced in a variety of embodiments and that the foregoing description and examples below are for purposes of illustration and not limitation of the claims that follow.

These and other aspects will become apparent from the following written description of the disclosure.

### DESCRIPTION OF DRAWINGS

FIG. 1 shows effect of the iADCs in the induction of human CXCL10 in cell culture supernates by ELISA kit (#DIP100, R&D);

FIG. 2 shows effect of the iADCs in the induction of IFN-3 by MAD-MB-231 human breast adenocarcinoma cells/THP-1 co-culture assay.

FIG. 3 shows the anti-tumor efficacy of test compounds in SK—OV-3 ovarian cancer xenograft model in CB17-SCID mice.

FIG. 4 shows safety assessment according to changes in body weight for test compounds in SK—OV-3 ovarian cancer xenograft model in CB17-SCID mice.

### EXAMPLES

The following Examples are provided to better illustrate the present disclosure. All parts and percentages are by weight and all temperatures are in degrees Celsius, unless explicitly stated otherwise. The following abbreviations have been used in the examples:

| | |
|---|---|
| NBS | N-Bromosuccinimide |
| EA | Ethyl acetate |
| ACN | Acetonitrile |
| THF | Tetrahydrofuran |
| DCM | Dichloromethane |
| DIEA | N,N-Diisopropylethylamine |
| DMF | Dimethylformamide |
| TEA | Triethylamine |
| TFA | Trifluoroacetic acid |
| FA | Formic acid |
| LDA | Lithium diisopropylamide |
| OXONE | PotassiumPeroxomonosulphate |
| NMI | 1-Methylimidazole |
| TCFH | N,N,N′,N′-Tetramethylchloroformamidinium |
| | hexafluorophosphate |
| NCS | N-Chlorosuccinimide |
| Pd(dppf)Cl₂ | [1,1′-Bis(diphenylphosphino)ferrocene]dichloropalladium |
| | (II) |
| CDI | N,N-Carbonyldiimidazole |
| RockPhos | Methanesulfonato(2-(di-t-butylphosphino)-3-methoxy-6- |
| Palladacycle | methyl-2′,4′,6′-tri-i-propyl-1,1′-biphenyl)(2′-amino-1,1′- |
| Gen.3 | biphenyl-2-yl)palladium(II) |
| MeOH | Methanol |
| EtOH | Ethanol |
| INT | Intermediate |
| h | hour(s) |
| aq | aqueous |
| sat | saturated |
| tol | toluene |
| TLC | Thin layer chromatography |
| Pre-TLC | Preparative thin layer chromatography |
| Prep-HPLC | Preparation high performance liquid chromatography |
| MS | Molecular sieve |

### SERIES 1-COMPOUNDS OF FORMULA Y-1, Y-2, Y-3, (A), (B), OR (C)

### INTERMEDIATES

### INT A1, INT A2 AND INT A3:

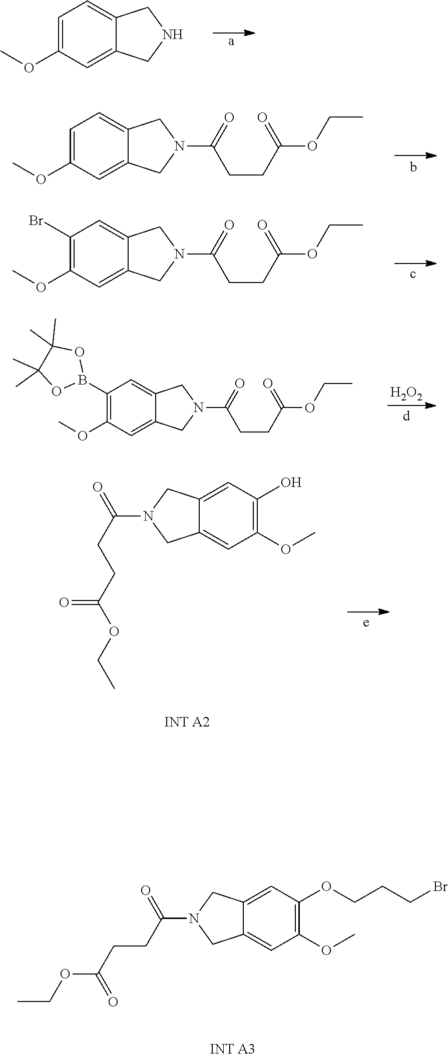

Step a: Succinic anhydride (4.40 g, 43.9681 mmol) was added to a solution of 5-methoxyisoindoline hydrochloride (4.89 g, 26.3399 mmol) and triethylamine (4.73 g, 46.7440 mmol) in ethanol (200 mL) at 20° C.

The reaction mixture was stirred for 1 h at 20° C. SOC₂ (20 mL) was added to the reaction mixture at 0° C., and stirred for 3 h at 20° C. The reaction mixture was evaporated under reduced pressure, and then diluted with EA (200 mL), and washed with water (100 mL) and brine (50 mL). The organics was dried over Na₂SO₄, filtered and evaporated under reduced pressure. There was ethyl 4-(5-methoxyisoindolin-2-yl)-4-oxo-butanoate (17.5 g, 31.5526 mmol, 119.7901% yield) obtained as a yellow solid.

¹H NMR (400 MHz, DMSO-d₆) δ 7.28-7.21 (m, 1H), 6.94 (s, 0.5H), 6.92 (s, 0.5H), 6.88 (s, 0.5H), 6.86 (s, 0.5H), 4.81 (s, 1H), 4.76 (s, 1H), 4.58 (s, 1H), 4.54 (s, 1H), 4.02-3.95 (m, 2H), 3.75 (s, 3H), 2.65-2.58 (m, 2H), 2.58-2.54 (m, 2H), 1.17 (t, J=3.5 Hz, 3H).

Step b: NBS (10.51 g, 59.0503 mmol) was added to ethyl 4-(5-methoxyisoindolin-2-yl)-4-oxo-butanoate in Tetrahydrofuran (100 mL) and Acetonitrile (100 mL) at 20° C. The reaction mixture was stirred overnight at 20° C.

The reaction mixture was evaporated under reduced pressure. The reaction mixture was concentrated and diluted with DCM (500 mL), washed with NaHCO₃ aq. (2×300 mL) and brine (200 mL). The organics was dried over Na₂SO₄, filtered and evaporated under reduced pressure. The residue was purified on silica gel column MeOH/DCM (0-10%). The pure fraction was concentrated and dried under vacuo. There was ethyl 4-(5-bromo-6-methoxy-isoindolin-2-yl)-4-oxo-butanoate (7.26 g, 20.3812 mmol, 64.5943% yield) obtained as a yellow solid.

¹H NMR (400 MHz, DMSO-d₆) δ 7.58 (s, 0.5H), 7.57 (s, 0.5H), 7.15 (s, 0.5H), 7.12 (s, 0.5H), 4.80 (s, 1H), 4.77 (s, 1H), 4.57 (s, 1H), 4.54 (s, 1H), 4.09-4.01 (m, 2H), 3.84 (s, 3H), 2.65-2.59 (m, 2H), 2.56-2.53 (m, 2H), 1.18 (t, J=7.1 Hz, 3H).

Step c: Potassium Acetate (3.74 g, 38.1080 mmol) was added to Pd(dppf)Cl₂ (0.72 g, 984.0050 mol), ethyl 4-(5-bromo-6-methoxy-isoindolin-2-yl)-4-oxo-butanoate (3.64 g, 10.2187 mmol) and 4,4,4′,4′,5,5,5′,5′-Octamethyl-2,2′-bi(1,3,2-dioxaborolane) (3.84 g, 15.1218 mmol) in 1,4-Dioxane (100 mL) at 20° C. The reaction mixture was stirred at 100° C. for 16 h. The reaction mixture was evaporated under reduced pressure. The reaction mixture was concentrated and diluted with DCM (500 mL), washed with NaHCO₃ aq. (2×300 mL) and brine (200 mL). The organics was dried over Na₂SO₄, filtered and evaporated under reduced pressure. The residue was purified on silica gel column EA/Hept (0-100%). The pure fraction was concentrated and dried under vacuo.

There was ethyl 4-(5-methoxy-6-(4,4,5,5-tetramethyl-1,3,2-dioxaborolan-2-yl)isoindolin-2-yl)-4-oxobutanoate (2.45 g, 6.0752 mmol, 59.7809% yield) obtained as a yellow solid.

¹H NMR (400 MHz, DMSO-d₆) δ 7.49 (s, 0.5H), 7.48 (s, 0.5H), 6.98 (s, 0.5H), 6.95 (s, 0.5H), 4.83 (s, 1H), 4.74 (s, 1H), 4.60 (s, 1H), 4.53 (s, 1H), 4.09-4.01 (m, 2H), 3.74 (t, J=6.2 Hz, 3H), 2.65-2.58 (m, 2H), 2.58-2.52 (m, 2H), 1.27 (s, 12H), 1.21-1.13 (m, 3H).

Step d: Sodium perborate tetrahydrate (1 g, 6.4994 mmol) was added to ethyl 4-(5-methoxy-6-(4,4,5,5-tetramethyl-1,3,2-dioxaborolan-2-yl)isoindolin-2-yl)-4-oxobutanoate (2.45 g, 6.0752 mmol) in Tetrahydrofuran (20 mL) and Water (20 mL) at 20° C. The reaction mixture was stirred for 1 h at 20° C. The reaction mixture was evaporated under reduced pressure. The reaction mixture was concentrated and diluted with DCM (500 mL), washed with NaHCO₃ aq. (2×300 mL) and brine (200 mL). The organics was dried over Na₂SO₄, filtered and evaporated under reduced pressure. The residue was purified on silica gel column MeOH/DCM (0-10%). The pure fraction was concentrated and dried under vacuo. There was ethyl 4-(5-hydroxy-6-methoxyisoindolin-2-yl)-4-oxobutanoate (1.37 g, 4.6708 mmol, 76.8818% yield) obtained as a yellow solid.

¹H NMR (400 MHz, DMSO-d₆) δ 8.97 (s, 0.5H), 8.96 (s, 0.5H), 6.91 (s, 0.5H), 6.89 (s, 0.5H), 6.73 (s, 0.5H), 6.73 (s, 0.5H), 4.71 (s, 1H), 4.69 (s, 1H), 4.50 (s, 1H), 4.47 (s, 1H), 4.08-4.00 (m, 2H), 3.75 (s, 3H), 2.64-2.58 (m, 2H), 2.56-2.53 (m, 2H), 1.18 (t, J=7.1 Hz, 3H).

Step e: To a solution of ethyl 4-(5-hydroxy-6-methoxy-isoindolin-2-yl)-4-oxo-butanoate (0.72 g, 2.4547 mmol) in N,N-Dimethylformamide (10 mL) was added 1,3-Dibromopropane (0.73 g, 3.6159 mmol) and potassium carbonate (0.86 g, 6.2226 mmol)₂. This mixture was stirred for 16 hours at 50° C. The reaction mixture was diluted with Ethyl acetate (100 mL), and washed sequentially with water (2×100 mL) and saturated brine (1×100 mL). The organic layer was dried over Na₂SO₄, filtered and evaporated to afford crude product. The crude product was purified by flash silica chromatography, elution gradient 0 to 100% Ethyl acetate in heptane. There was ethyl 4-(5-(3-bromopropoxy)-6-methoxyisoindolin-2-yl)-4-oxobutanoate (0.43 g, 1.0379 mmol, 42.2829% yield) obtained as a white solid.

¹H NMR (400 MHz, DMSO-d₆) δ 7.01-6.93 (m, 2H), 4.75 (s, 2H), 4.53 (s, 2H), 4.10-4.00 (m, 4H), 3.76 (s, 3H), 3.71-3.62 (m, 2H), 2.65-2.58 (m, 2H), 2.58-2.53 (m, 2H), 2.28-2.18 (m, 2H), 1.18 (t, J=7.1 Hz, 3H).

### INT A4: ethyl 4-(4-fluoro-5-hydroxy-6-methoxyisoindolin-2-yl)-4-oxobutanoate

### Step a: 7-fluoro-6-methoxy-2-[(4methoxyphenyl) methyl] isoindolin-1-one

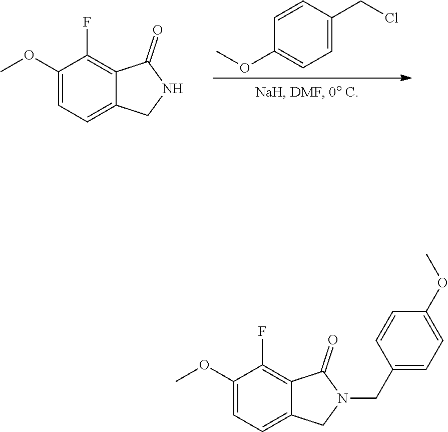

4-Methoxybenzylchloride (4.141 g, 26.4416 mmol) was added to a solution of NaH (1.000 g, 25.0024 mmol) and 7-fluoro-6-methoxyisoindolin-1-one (4.336 g, 23.9342 mmol) in DMF (100 mL) at 0° C. under N₂ atmosphere.

The reaction mixture was stirred for 3 h and warmed up to 20° C. naturally. The reaction mixture was quenched with adding to water (300 mL) at 20° C., extracted with EA (3×200 mL) and washed with brine (150 mL). The organics dried over Na₂SO₄, filtered and evaporated under reduced pressure. The residue was purified on silica gel column EA/n-Hex (0-100%). The pure fraction was concentrated and dried under vacuo. There was 7-fluoro-6-methoxy-2-[(4methoxyphenyl) methyl] isoindolin-1-one (6.51 g, 21.6055 mmol, 90.2706% yield) obtained as a yellow solid. LCMS: (ESI, m/z): [M+H]+=302.110. ¹H NMR (400 MHz, DMSO-d₆) δ 7.39 (t, J=7.9 Hz, 1H), 7.27 (d, J=8.2 Hz, 1H), 7.22 (d, J=8.2 Hz, 2H), 6.91 (d, J=8.3 Hz, 2H), 4.60 (s, 2H), 4.25 (s, 2H), 3.87 (s, 3H), 3.73 (s, 3H).

### Step b: 4-fluoro-5-methoxy-2[(4methoxyphenyl)methyl]isoindoline

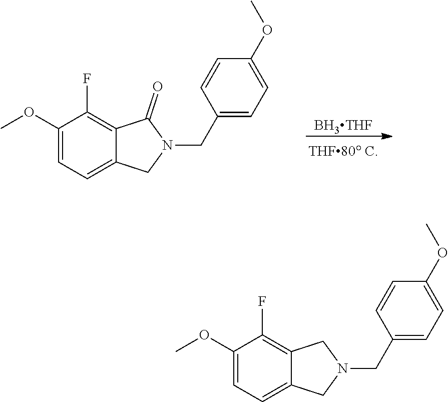 [SMILES: COc1ccc(CN2Cc3ccc(OC)c(F)c3C2=O)cc1 ; COc1ccc(CN2Cc3ccc(OC)c(F)c3C2)cc1 ; "BH3·THF"]

Borane-tetrahydrofuran complex (120 mL, 120 mmol) was added to a solution of 7-fluoro-6-methoxy-2-(4-methoxybenzyl)isoindolin-1-one (6.17 g, 20.4771 mmol) in THF (60 mL) at 20° C. under N₂ atmosphere. The reaction mixture was heated to 80° C. and stirred for 5 h. The reaction mixture was quenched with pouring into MeOH (300 mL) at 20° C. and evaporated under reduced pressure. The residue was diluted with MeOH (100 mL). The precipitate was collected by filtration, washed with MeOH (50 mL). The filter cake was dried under vacuo. There was 4-fluoro-5-methoxy-2[(4methoxyphenyl)methyl]isoindoline (3.854 g, 13.4132 mmol, 65.5036% yield) obtained as a white solid. LCMS: (ESI, m/z): [M+H]⁺=288.130. ¹H NMR (400 MHz, DMSO-d₆) δ 7.41 (d, J=8.0 Hz, 2H), 7.06-6.93 (m, 2H), 6.88 (d, J=8.0 Hz, 2H), 4.53-4.37 (m, 2H), 4.22-4.08 (m, 4H), 3.79 (s, 3H), 3.74 (s, 3H).

### Step c: ethyl 4-(4-fluoro-5-methoxy-isoindolin-2-yl)-4-oxo-butanoate

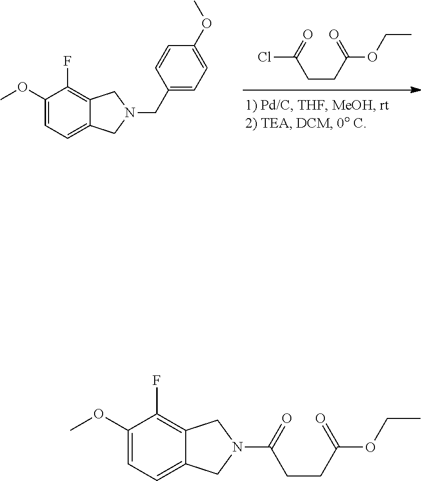

Pd/C (2.17 g, 2.0391 mmol) was added to a solution of 4-fluoro-5-methoxy-2-(4-methoxybenzyl)isoindoline (3.36 g, 11.6940 mmol) in THF (20 mL) and MeOH (20 mL) at 20° C. The reaction mixture was stirred overnight at 20° C. under H₂ atmosphere. The precipitate was collected by filtration, washed with MeOH (50 mL). The filtrate was evaporated under reduced pressure. Ethyl Succinyl Chloride (5.05 g, 30.6830 mmol) was added to a solution of the residue and TEA (3.91 g, 38.6404 mmol) in DCM (50 mL) at 0° C. under N₂ atmosphere. The reaction mixture was stirred for 1 h at 20° C. The reaction mixture was quenched with adding of water (50 mL) at 20° C., extracted with DCM (3×50 mL) and washed with brine (50 mL). The organics dried over Na₂SO₄, filtered and evaporated under reduced pressure. The residue was purified on silica gel column EA/n-Hex (0-45%). The pure fraction was concentrated and dried under vacuo. There was ethyl 4-(4-fluoro-5-methoxy-isoindolin-2-yl)-4-oxo-butanoate (2.619 g, 8.8688 mmol, 75.8406% yield) obtained as a yellow solid. LCMS: (ESI, m/z): [M+H]⁺=296.120. ¹H NMR (400 MHz, DMSO-d₆) δ 7.16-7.09 (m, 2H), 4.91 (s, 1H), 4.81 (s, 1H), 4.64 (s, 1H), 4.58 (s, 1H), 4.05 (q, J=7.2 Hz, 2H), 3.84 (s, 3H), 2.73-2.53 (m, 4H), 1.18 (t, J=7.2 Hz, 3H).

### Step d: ethyl 4-(4-fluoro-5-hydroxy-isoindolin-2-yl)-4-oxo-butanoate

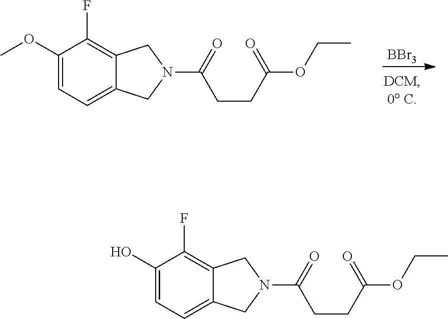

Tribromoboron (20 mL, 20 mmol) was added to a solution of ethyl 4-(4-fluoro-5-methoxyisoindolin-2-yl)-4-oxobutanoate (2.597 g, 8.7943 mmol) in DCM (40 mL) at 0° C. under N₂ atmosphere. The reaction mixture was stirred for 2 h at 20° C. The reaction mixture was quenched with adding to EtOH (100 mL) at 20° C. The reaction mixture was evaporated under reduced pressure and diluted with H₂O (200 mL), extracted with EA (2×100 mL) and washed with brine (100 mL). The organics dried over Na₂SO₄, filtered and evaporated under reduced pressure.

There was ethyl 4-(4-fluoro-5-hydroxy-isoindolin-2-yl)-4-oxo-butanoate (2.408 g, 8.5609 mmol, 97.3461% yield) obtained as a yellow solid. LCMS: (ESI, m/z): [M+H]⁺=282.110. ¹H NMR (400 MHz, DMSO-d₆) δ 9.83 (s, 1H), 6.99-6.85 (m, 2H), 4.88 (s, 1H), 4.77 (s, 1H), 4.62 (s, 1H), 4.54 (s, 1H), 4.05 (q, J=7.2 Hz, 2H), 2.66-2.53 (m, 4H), 1.18 (t, J=7.2 Hz, 3H).

### Step e: ethyl 4-(6-bromo-4-fluoro-5-hydroxy-isoindolin-2-yl)-4-oxo-butanoate

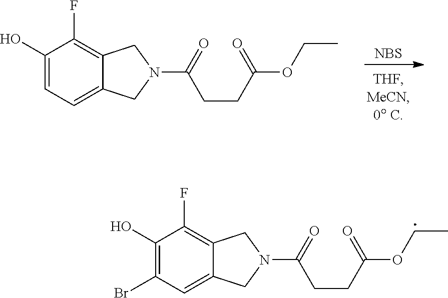

NBS (1.194 g, 6.7085 mmol) was added to a solution of ethyl 4-(4-fluoro-5-hydroxy-isoindolin-2-yl)-4-oxo-butanoate (2.106 g, 7.4872 mmol) in MeCN (20 mL) and THF (20 mL) at 0° C. under N₂ atmosphere. The reaction mixture was stirred for 2 h and warmed up to 20° C. naturally. The precipitate was collected by filtration. The filter cake was dried under vacuo. There was ethyl 4-(6-bromo-4-fluoro-5-hydroxy-isoindolin-2-yl)-4-oxo-butanoate (2.84 g, 7.8851 mmol, 105.3134% yield) obtained as a white solid. LCMS: (ESI, m/z): [M+H]⁺=359.010. ¹H NMR (400 MHz, DMSO-d₆) δ 10.41 (s, 1H), 7.36 (s, 0.5H), 7.35 (s, 0.5H), 4.87 (s, 1H), 4.78 (s, 1H), 4.60 (s, 1H), 4.55 (s, 1H), 4.14-3.97 (q, J=7.2 Hz, 2H), 2.70-2.52 (m, 4H), 1.18 (t, J=7.2 Hz, 3H).

### Step f: ethyl 4-(6-bromo-4-fluoro-5-(methoxymethoxy)isoindolin-2-yl)-4-oxobutanoate

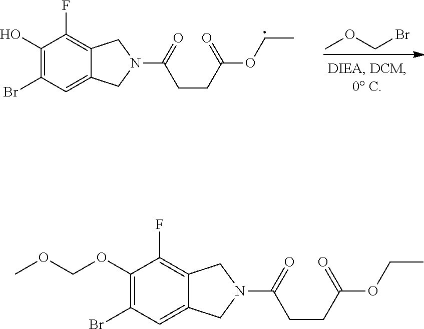

Bromomethyl methyl ether (1.20 g, 9.6028 mmol) was added to a solution of ethyl 4-(6-bromo-4-fluoro-5-hydroxy-isoindolin-2-yl)-4-oxo-butanoate (2.23 g, 6.1914 mmol) and DIEA (2.55 g, 19.7304 mmol) in DCM (50 mL) at 0° C. under N₂ atmosphere. The reaction mixture was stirred for 2 h and warmed up to 20° C. naturally. The reaction mixture was quenched with adding of water (50 mL) at 20° C., extracted with DCM (3×50 mL) and washed with brine (50 mL). The organics dried over Na₂SO₄, filtered and evaporated under reduced pressure. The residue was purified on C18 column ACN/H₂O (0.1% FA)(0-50%). The pure fraction was concentrated and dried under vacuo. There was ethyl 4-(6-bromo-4-fluoro-5-(methoxymethoxy)isoindolin-2-yl)-4-oxobutanoate (1.809 g, 4.4752 mmol, 72.2806% yield) obtained as a yellow solid. LCMS: (ESI, m/z): [M+H]⁺=403.140. ¹H NMR (400 MHz, DMSO-d₆) δ 7.52 (s, 0.5H), 7.51 (s, 0.5H), 5.16 (s, 2H), 4.90 (s, 1H), 4.85 (s, 1H), 4.63 (s, 1H), 4.61 (s, 1H), 4.05 (q, J=7.2 Hz, 2H), 3.54 (s, 3H), 2.72-2.53 (m, 4H), 1.24-1.12 (t, J=7.2 Hz, 3H).

### Step g: ethyl 4-(4-fluoro-6-hydroxy-5-(methoxymethoxy)isoindolin-2-yl)-4-oxobutanoate

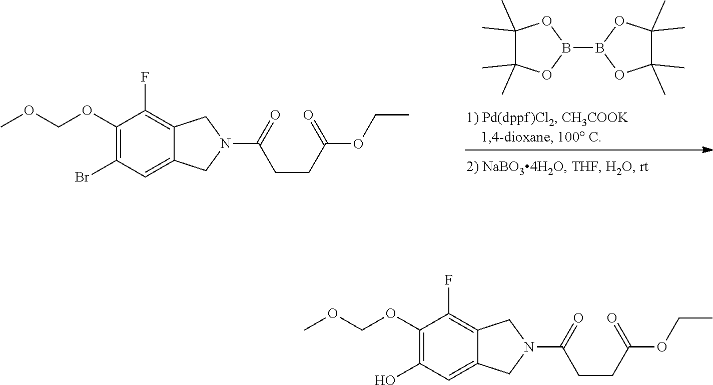

Pd(dppf)Cl₂ (0.228 g, 311.6016 mol), 4, 4,4′,4′,5,5,5′,5′-Octamethyl-2,2′-bi(1,3,2-dioxaborolane) (1.170 g, 4.6074 mmol) and Potassium Acetate (0.743 g, 7.5706 mmol) was added to a solution of ethyl 4-(6-bromo-4-fluoro-5-(methoxymethoxy)isoindolin-2-yl)-4-oxobutanoate (0.735 g, 1.8183 mmol) in 1,4-Dioxane (20 mL) at 20° C. under N₂ atmosphere. The reaction mixture was heated to 100° C. and stirred for 12 h. The reaction mixture was quenched with adding of water (150 mL) at 20° C., extracted with EA (3×150 mL) and washed with brine (50 mL). The organics dried over Na₂SO₄, filtered and evaporated under reduced pressure. Sodium perborate (0.91 g, 5.9145 mmol) was added to a solution of the residue in THF (10 mL) and H₂O (10 mL) at 20° C. under N₂ atmosphere. The reaction mixture was stirred for 1 h at 20° C. The reaction mixture was evaporated under reduced pressure. The residue was purified on C18 column ACN/H₂O (0.1% FA)(0-38%). The pure fraction was concentrated and dried under vacuo. There was ethyl 4-(4-fluoro-6-hydroxy-5-(methoxymethoxy)isoindolin-2-yl)-4-oxobutanoate (0.365 g, 1.0693 mmol, 58.8% yield) obtained as a white solid. LCMS: (ESI, m/z): [M+H]⁺=342.130. ¹H NMR (400 MHz, DMSO-d₆) δ 9.97 (s, 1H), 6.68 (s, 1H), 5.04 (s, 2H), 4.80 (s, 1H), 4.76 (s, 1H), 4.55 (s, 1H), 4.53 (s, 1H), 4.05 (q, J=7.2 Hz, 2H), 3.47 (s, 3H), 2.67-2.51 (m, 4H), 1.18 (t, J=7.2 Hz, 3H).

### Step h: ethyl4-(4-fluoro-5-hydroxy-6-methoxy-isnindolin-2-yl)-4-nxo-hutanoate

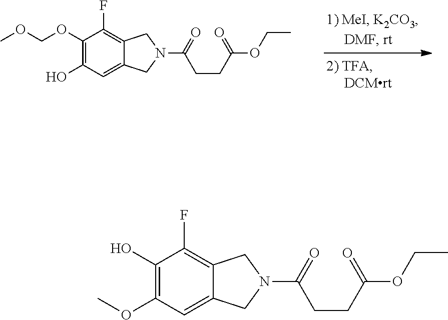

Methyl Iodidle (0.345 g, 2.4306 mmol) was added to a mixture of Potassium carbonate (0.448 g, 3.2415 mmol) and ethyl 4-(4-fluoro-6-hydroxy-5-(methoxymethoxy)isoindolin-2-yl)-4-oxobutanoate (0.328 g, 960.9441 μmol) in DMF (10 mL) at 20° C. under N₂ atmosphere. The reaction mixture was stirred for 1 h at 20° C. The reaction mixture was quenched with adding of water (50 mL) at 20° C., extracted with DCM (3×50 mL) and washed with brine (50 mL). The organics dried over Na₂SO₄, filtered and evaporated under reduced pressure. Trifluoroacetic acid (2 mL) was added to a solution of the residue (0.446 g, 1.2551 mmol) in DCM (5 mL) at 20° C. under N₂ atmosphere. The reaction mixture was stirred for 2 h at 20° C. The reaction mixture was evaporated under reduced pressure. The residue was purified on C18 column ACN/H₂O (0-80%). The pure fraction was concentrated and dried under vacuo. There was ethyl 4-(4-fluoro-5-hydroxy-6-methoxy-isoindolin-2-yl)-4-oxo-butanoate (0.274 g, 880.1663 mol, 91.6% yield) obtained as a white solid. LCMS: (ESI, m/z): [M+H]⁺=312.12.

¹H NMR (400 MHz, DMSO-d₆) δ 9.17 (s, 1H), 6.83 (s, 0.5H), 6.80 (s, 0.5H), 4.82 (s, 1H), 4.77 (s, 1H), 4.57 (s, 1H), 4.54 (s, 1H), 4.05 (q, J=7.2 Hz, 2H), 3.80 (d, J=2.0 Hz, 3H), 2.68-2.51 (m, 4H), 1.18 (t, J=7.2 Hz, 3H).

### Example I-1

### 4,4′-((propane-1,3-diylbis(oxy))bis(6-methoxyisoindoline-5,2-diyl))bis(4-oxobutanoic acid)

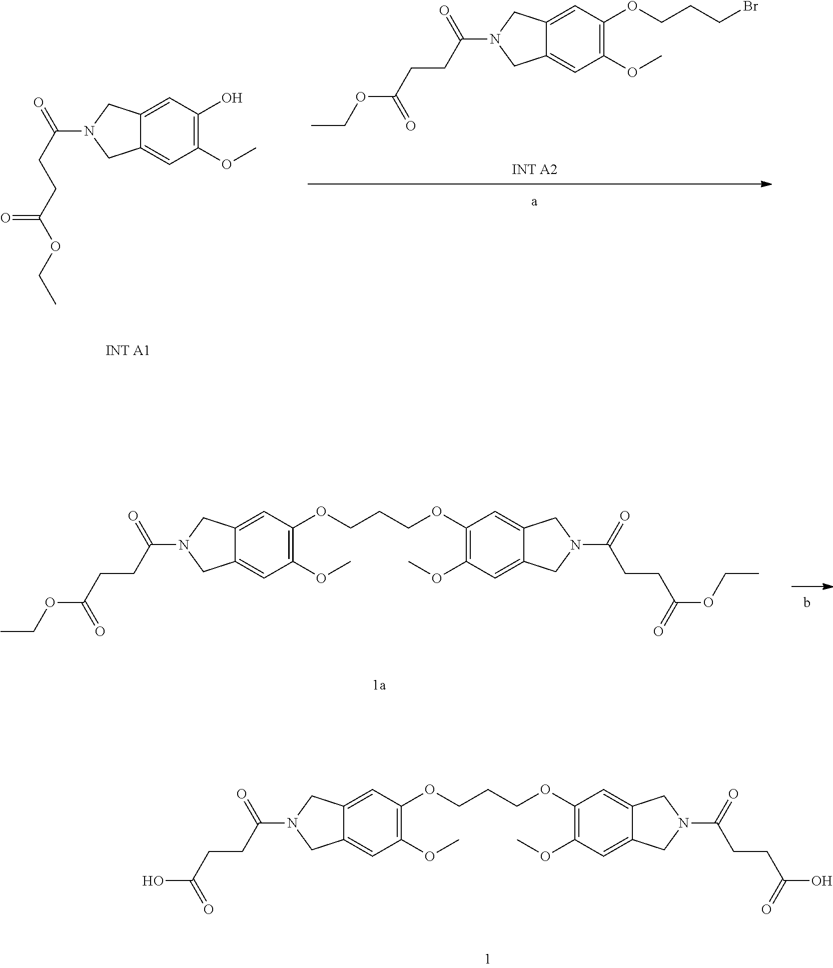

Step a: Potassium carbonate (0.240 g, 1.7365 mmol) was added to a solution of INT A3 (0.240 g, 579.3045 μmol) and INT A2 (0.169 g, 576.1737 mol) in N,N-Dimethylformamide (10 mL) at 20° C. The reaction mixture was heated to 50° C. and stirred overnight. The reaction mixture was evaporated under reduced pressure. The reaction mixture was concentrated and diluted with DCM (500 mL), washed with NaHCO₃ aq. (2×300 mL) and brine (200 mL). The organics dried over Na₂SO₄, filtered and evaporated under reduced pressure. The residue was purified on silica gel column MeCN/water (0-100%). The pure fraction was concentrated and dried under vacuo. There was compound la (0.185 g, 295.2007 mol, 50.9578% yield) obtained as a yellow solid.

¹H NMR (400 MHz, DMSO-d₆) δ 7.01-6.93 (m, 4H), 4.75 (s, 2H), 4.73 (s, 2H), 4.53 (s, 2H), 4.51 (s, 2H), 4.14-4.08 (m, 4H), 4.05 (q, J=7.1 Hz, 4H), 3.74 (t, J=1.9 Hz, 6H), 2.65-2.58 (m, 4H), 2.58-2.53 (m, 4H), 2.35-2.30 (m, 2H), 1.18 (t, J=7.1 Hz, 6H).

Step b: A solution of LiOH (0.042 g, 1.7538 mmol) in Water (5 mL) was added to a solution of compound la (0.182 g, 290.4138 mol) in Tetrahydrofura (20 mL) at 20° C. The reaction mixture was stirred at 20° C. The reaction mixture was adjusted pH=7 with adding of HCl (1 M) in water. The reaction mixture was concentrated under reduced pressure. The residue was purified on HPLC. The pure fraction was concentrated and dried under vacuo. There was compound 1 (0.104 g, 182.2687 mol, 62.7617% yield) obtained as a white solid.

¹H NMR (400 MHz, DMSO-d₆) δ 6.97-6.89 (m, 3H), 6.83 (s, 0.5H), 6.81 (s, 0.5H), 4.76 (s, 1H), 4.73 (s, 1H), 4.68 (s, 1H), 4.56 (s, 1H), 4.49 (s, 2H), 4.45 (s, 1H), 4.37 (s, 1H), 4.16-4.06 (m, 4H), 3.74 (d, J=5.4 Hz, 6H), 2.49-2.41 (m, 4H), 2.40-2.30 (m, 4H), 2.16-2.08 (m, 2H).

### Example I-2

### 4-(6-(3-((2-(3-carboxypropanoyl)-6-methoxyisoindolin-5-yl)oxy)propoxy)-4-fluoro-5-methoxyisoindolin-2-yl)-4-oxobutanoic acid

### Step a: 7-fluoro-6-methoxy-2-[(4methoxyphenyl) methyl] isoindolin-1-one

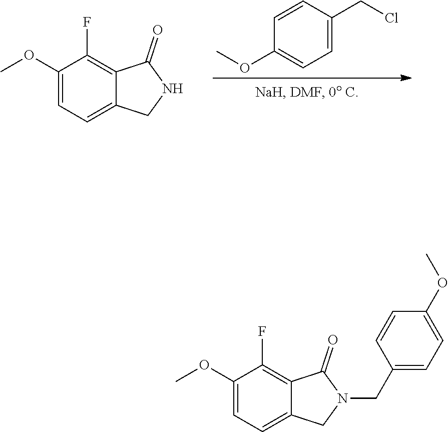

4-Methoxybenzylchloride (4.141 g, 26.4416 mmol) was added to a solution of NaH (1.000 g, 25.0024 mmol) and 7-fluoro-6-methoxyisoindolin-1-one (4.336 g, 23.9342 mmol) in DMF (100 mL) at 0° C. under N₂ atmosphere. The reaction mixture was stirred for 3 h and warmed up to 20° C. naturally. The reaction mixture was quenched with adding to water (300 mL) at 20° C., extracted with EA (3×200 mL) and washed with brine (150 mL). The organics dried over Na₂SO₄, filtered and evaporated under reduced pressure. The residue was purified on silica gel column EA/n-Hex (0-100%). The pure fraction was concentrated and dried under vacuo. There was 7-fluoro-6-methoxy-2-[(4methoxyphenyl) methyl] isoindolin-1-one (6.51 g, 21.6055 mmol, 90.2706% yield) obtained as a yellow solid. LCMS: (ESI, m/z): [M+H]⁺=302.110. ¹H NMR (400 MHz, DMSO-d₆) δ 7.39 (t, J=7.9 Hz, 1H), 7.27 (d, J=8.2 Hz, 1H), 7.22 (d, J=8.2 Hz, 2H), 6.91 (d, J=8.3 Hz, 2H), 4.60 (s, 2H), 4.25 (s, 2H), 3.87 (s, 3H), 3.73 (s, 3H).

### Step b: 4-fluoro-5-methoxy-2[(4methoxyphenyl)methyl]isoindoline

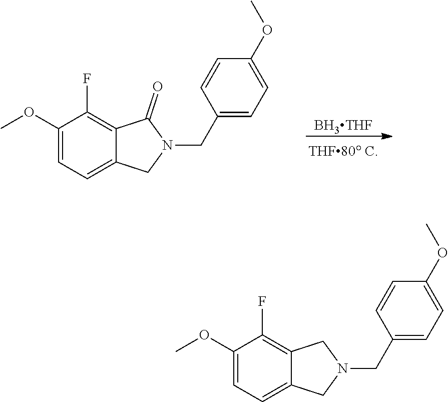 [SMILES: COc1ccc(CN2Cc3ccc(OC)c(F)c3C2=O)cc1 ; COc1ccc(CN2Cc3ccc(OC)c(F)c3C2)cc1 ; "BH3·THF"]

Borane-tetrahydrofuran complex (120 mL, 120 mmol) was added to a solution of 7-fluoro-6-methoxy-2-(4-methoxybenzyl)isoindolin-1-one (6.17 g, 20.4771 mmol) in THF (60 mL) at 20° C. under N₂ atmosphere. The reaction mixture was heated to 80° C. and stirred for 5 h. The reaction mixture was quenched with pouring into MeOH (300 mL) at 20° C. and evaporated under reduced pressure. The residue was diluted with MeOH (100 mL). The precipitate was collected by filtration, washed with MeOH (50 mL). The filter cake was dried under vacuo. There was 4-fluoro-5-methoxy-2[(4methoxyphenyl)methyl]isoindoline (3.854 g, 13.4132 mmol, 65.5036% yield) obtained as a white solid. LCMS: (ESI, m/z): [M+H]⁺=288.130. ¹H NMR (400 MHz, DMSO-d₆) δ 7.41 (d, J=8.0 Hz, 2H), 7.06-6.93 (m, 2H), 6.88 (d, J=8.0 Hz, 2H), 4.53-4.37 (m, 2H), 4.22-4.08 (m, 4H), 3.79 (s, 3H), 3.74 (s, 3H).

### Step c: ethyl 4-(4-fluoro-5-methoxy-isoindolin-2-yl)-4-oxo-butanoate

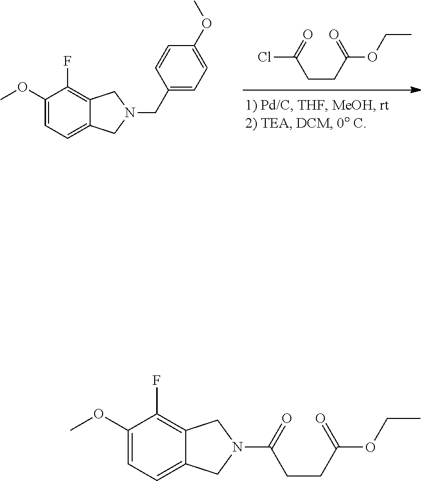

Pd/C (2.17 g, 2.0391 mmol) was added to a solution of 4-fluoro-5-methoxy-2-(4-methoxybenzyl)isoindoline (3.36 g, 11.6940 mmol) in THF (20 mL) and MeOH (20 mL) at 20° C. under H₂ atmosphere. The reaction mixture was stirred overnight at 20° C. The precipitate was collected by filtration, washed with MeOH (50 mL). The filtrate was evaporated under reduced pressure. Ethyl Succinyl Chloride (5.05 g, 30.6830 mmol) was added to a solution of the residue and TEA (3.91 g, 38.6404 mmol) in DCM (50 mL) at 0° C. under N₂ atmosphere. The reaction mixture was stirred for 1 h at 20° C. The reaction mixture was quenched with adding of water (50 mL) at 20° C., extracted with DCM (3×50 mL) and washed with brine (50 mL). The organics dried over Na₂SO₄, filtered and evaporated under reduced pressure. The residue was purified on silica gel column EA/n-Hex (0-45%). The pure fraction was concentrated and dried under vacuo. There was ethyl 4-(4-fluoro-5-methoxy-isoindolin-2-yl)-4-oxo-butanoate (2.619 g, 8.8688 mmol, 75.8406% yield) obtained as a yellow solid. LCMS: (ESI, m/z): [M+H]⁺=296.120. ¹H NMR (400 MHz, DMSO-d₆) δ 7.16-7.09 (m, 2H), 4.91 (s, 1H), 4.81 (s, 1H), 4.64 (s, 1H), 4.58 (s, 1H), 4.05 (q, J=7.2 Hz, 2H), 3.84 (s, 3H), 2.73-2.53 (m, 4H), 1.18 (t, J=7.2 Hz, 3H).

### Step d: ethyl 4-(4-fluoro-5-hydroxy-isoindolin-2-yl)-4-oxo-butanoate

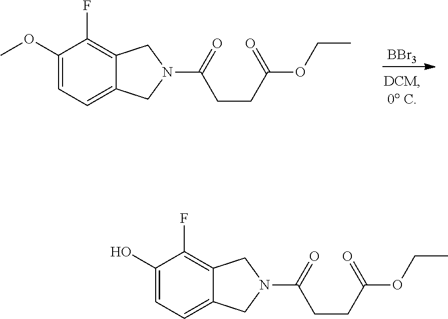

Tribromoboron (20 mL, 20 mmol) was added to a solution of ethyl 4-(4-fluoro-5-methoxyisoindolin-2-yl)-4-oxobutanoate (2.597 g, 8.7943 mmol) in DCM (40 mL) at 0° C. under N₂ atmosphere. The reaction mixture was stirred for 2 h at 20° C. The reaction mixture was quenched with adding to EtOH (100 mL) at 20° C., The reaction mixture was evaporated under reduced pressure and diluted with H₂O (200 mL), extracted with EA (2×100 mL) and washed with brine (100 mL). The organics dried over Na₂SO₄, filtered and evaporated under reduced pressure.

There was ethyl 4-(4-fluoro-5-hydroxy-isoindolin-2-yl)-4-oxo-butanoate (2.408 g, 8.5609 mmol, 97.3461% yield) obtained as a yellow solid. LCMS: (ESI, m/z): [M+H]⁺=282.110. ¹H NMR (400 MHz, DMSO-d₆) δ 9.83 (s, 1H), 6.99-6.85 (m, 2H), 4.88 (s, 1H), 4.77 (s, 1H), 4.62 (s, 1H), 4.54 (s, 1H), 4.05 (q, J=7.2 Hz, 2H), 2.66-2.53 (m, 4H), 1.18 (t, J=7.2 Hz, 3H).

### Step e: ethyl 4-(6-bromo-4-fluoro-5-hydroxy-isoindolin-2-yl)-4-oxo-butanoate

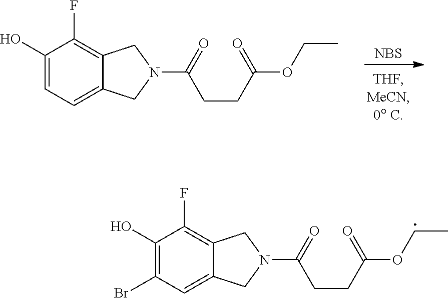

NBS (1.194 g, 6.7085 mmol) was added to a solution of ethyl 4-(4-fluoro-5-hydroxy-isoindolin-2-yl)-4-oxo-butanoate (2.106 g, 7.4872 mmol) in MeCN (20 mL) and THF (20 mL) at 0° C. under N₂ atmosphere. The reaction mixture was stirred for 2 h and warmed up to 20° C. naturally. The precipitate was collected by filtration. The filter cake was dried under vacuo. There was ethyl 4-(6-bromo-4-fluoro-5-hydroxy-isoindolin-2-yl)-4-oxo-butanoate (2.84 g, 7.8851 mmol, 105.3134% yield) obtained as a white solid. LCMS: (ESI, m/z): [M+H]⁺=359.010. ¹H NMR (400 MHz, DMSO-d₆) δ 10.41 (s, 1H), 7.36 (s, 0.5H), 7.35 (s, 0.5H), 4.87 (s, 1H), 4.78 (s, 1H), 4.60 (s, 1H), 4.55 (s, 1H), 4.14-3.97 (q, J=7.2 Hz, 2H), 2.70-2.52 (m, 4H), 1.18 (t, J=7.2 Hz, 3H).

### Step f: ethyl 4-(6-bromo-4-fluoro-5-methoxy-isoindolin-2-yl)-4-oxo-butanoate

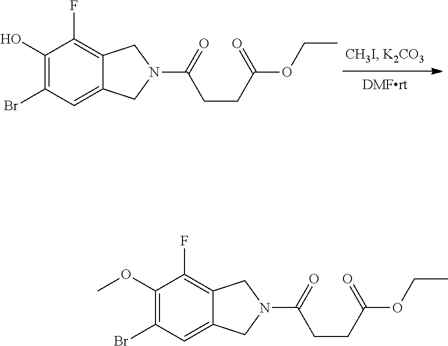

Methyl Iodidle (0.477 g, 3.3606 mmol) was added to a mixture of ethyl 4-(6-bromo-4-fluoro-5-hydroxyisoindolin-2-yl)-4-oxobutanoate (0.463 g, 1.2855 mmol) and Potassium carbonate (0.779 g, 5.6365 mmol) in DMF (5 mL) at 20° C. under N₂ atmosphere. The reaction mixture was stirred for 2 h at 25° C. The mixture was purified on C18 column ACN/H₂O (0.1% FA)(0-50%). The pure fraction was concentrated and dried under vacuo. There was ethyl 4-(6-bromo-4-fluoro-5-methoxy-isoindolin-2-yl)-4-oxo-butanoate (0.326 g, 871.1884 mol, 67.7711% yield) obtained as a white solid. LCMS: (ESI, m/z): [M+H]⁺=374.030. ¹H NMR (400 MHz, DMSO-d₆) δ 7.50 (s, 0.5H), 7.49 (s, 0.5H), 4.90 (s, 1H), 4.84 (s, 1H), 4.63 (s, 1H), 4.61 (s, 1H), 4.05 (q, J=7.1 Hz, 2H), 3.86 (s, 3H), 2.70-2.59 (m, 2H), 2.58-2.52 (m, 2H), 1.18 (t, J=7.1 Hz, 3H).

### Step g: [2-(4-ethoxy-4-oxo-butanoyl)-7-fluoro-6-methoxy-isoindolin-5-yl]boronic acid

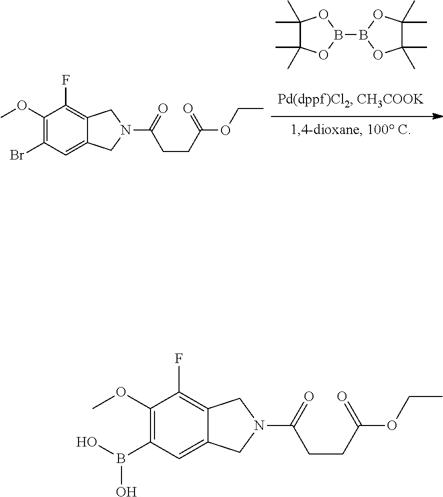

[1,1′-Bis(diphenylphosphino)ferrocene]dichloropalladium(II) (0.137 g, 187.2343 mol), 4,4,4′,4′,5,5,5′,5′-Octamethyl-2,2′-bi(1,3,2-dioxaborolane) (0.648 g, 2.5518 mmol) and Potassium Acetate (0.362 g, 3.6885 mmol) was added to a solution of ethyl 4-(6-bromo-4-fluoro-5-methoxy-isoindolin-2-yl)-4-oxo-butanoate (0.331 g, 884.5502 mol) in 1,4-Dioxane (5 mL) at 20° C. under N₂ atmosphere. The reaction mixture was heated to 90° C. and stirred overnight. The reaction mixture was diluted with EA (100 mL). The precipitate was collected by filtration, washed with EA (1×100 mL). The combined filtrate was evaporated under reduced pressure. The residue was purified on C18 column ACN/H₂O (0.1% FA)(0-35%). The pure fraction was concentrated and dried under vacuo. There was [2-(4-ethoxy-4-oxo-butanoyl)-7-fluoro-6-methoxy-isoindolin-5-yl]boronic acid (0.112 g, 330.2636 mol, 37.3369% yield) obtained as a colorless oil. LCMS: (ESI, m/z): [M+H]- =340.130.

### Step h: ethyl 4-(4-fluoro-6-hydroxy-5-methoxy-isoindolin-2-yl)-4-oxo-butanoate

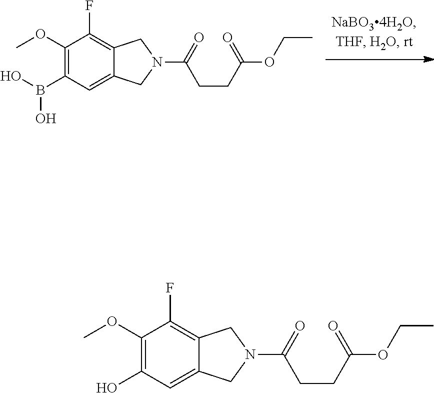

Sodium perborate tetrahydrate (0.098 g, 636.9435 mol) was added to a solution of [2-(4-ethoxy-4-oxo-butanoyl)-7-fluoro-6-methoxy-isoindolin-5-yl]boronic acid (0.105 g, 309.6221 mol) in THF (5 mL) and H₂O (5 mL) at 20° C. The reaction mixture was stirred for 1 h at 20° C. The reaction mixture was purified on C18 column ACN/H₂O (0.1% FA)(0-33%). The pure fraction was concentrated and dried under vacuo. There was ethyl 4-(4-fluoro-6-hydroxy-5-methoxy-isoindolin-2-yl)-4-oxo-butanoate (0.082 g, 263.4074 mol, 85.0739% yield) example obtained as a white solid. LCMS: (ESI, m/z): [M−H]—=310.120. ¹H NMR (400 MHz, DMSO-d₆) δ 9.82 (s, 1H), 6.66 (s, 1H), 4.79 (s, 1H), 4.75 (s, 1H), 4.54 (s, 1H), 4.52 (s, 1H), 4.05 (q, J=7.1 Hz, 2H), 3.77 (s, 3H), 2.65-2.57 (m, 2H), 2.56-2.52 (m, 2H), 1.18 (t, J=7.1 Hz, 3H).

### Step i: ethyl 4-(6-(3-((2-(4-ethoxy-4-oxobutanoyl)-6-methoxyisoindolin-5-yl)oxy)propoxy)-4-fluoro-5-methoxyisoindolin-2-yl)-4-oxobutanoate

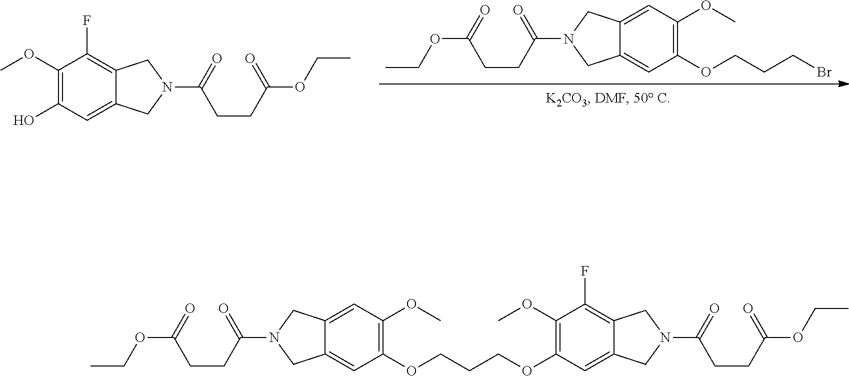

Potassium carbonate (0.104 g, 752.5026 mol) was added to a solution of ethyl 4-(4-fluoro-6-hydroxy-5-methoxy-isoindolin-2-yl)-4-oxo-butanoate (0.077 g, 247.3459 mol) and ethyl 4-(5-(3-bromopropoxy)-6-methoxyisoindolin-2-yl)-4-oxobutanoate (0.101 g, 243.7906 mol) in DMF (4 mL) at 20° C. under N₂ atmosphere.

The reaction mixture was stirred overnight at 50° C. The reaction mixture was purified on C18 column ACN/H₂O (0.1% FA)(0-50%). The pure fraction was concentrated and dried by lyophilization. There was ethyl 4-(6-(3-((2-(4-ethoxy-4-oxobutanoyl)-6-methoxyisoindolin-5-yl)oxy)propoxy)-4-fluoro-5-methoxyisoindolin-2-yl)-4-oxobutanoate (0.069 g, 107.0293 mol, 43.2711% yield) obtained as a white solid. LCMS: (ESI, m/z): [M+H]=645.270. ¹H NMR (400 MHz, DMSO-d₆) δ 7.05-6.91 (m, 3H), 4.84 (s, 1H), 4.79 (s, 1H), 4.75 (s, 1H), 4.73 (s, 1H), 4.58 (s, 1H), 4.57 (s, 1H), 4.53 (s, 1H), 4.51 (s, 1H), 4.19 (s, 2H), 4.12 (t, J=6.5 Hz, 2H), 4.05 (q, J=7.1 Hz, 4H), 3.78 (s, 3H), 3.74 (s, 3H), 2.69-2.58 (m, 4H), 2.57-2.52 (m, 4H), 2.25-2.14 (m, 2H), 1.18 (t, J=7.1 Hz, 6H).

### Step j: 4-(6-(3-((2-(3-carboxypropanoyl)-6-methoxyisoindolin-5-yl)oxy)propoxy)-4-fluoro-5-methoxyisoindolin-2-yl)-4-xobutanoic acid

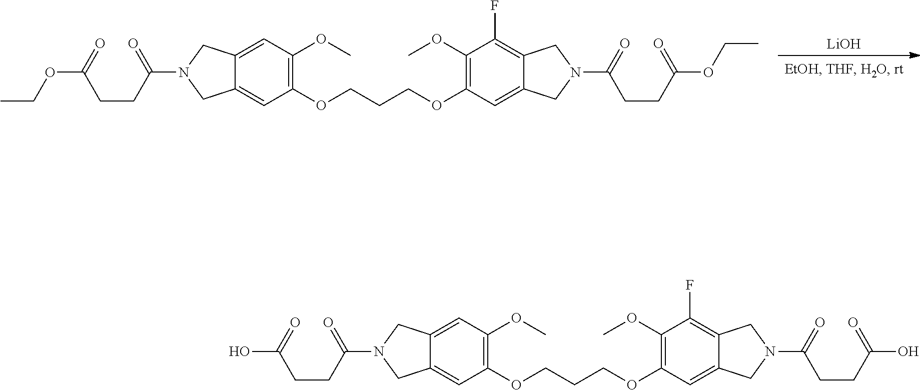 [SMILES: CCOC(=O)CCC(=O)N1Cc2cc(OC)c(OCCCOc3cc4c(c(F)c3OC)CN(C(=O)CCC(=O)OCC)C4)cc2C1 ; "LiOH" ; COc1cc2c(cc1OCCCOc1cc3c(c(F)c1OC)CN(C(=O)CCC(=O)O)C3)CN(C(=O)CCC(=O)O)C2]

LiOH (0.050 g, 2.0878 mmol) was added to a solution of ethyl 4-(6-(3-((2-(4-ethoxy-4-oxobutanoyl)-6-methoxyisoindolin-5-yl)oxy)propoxy)-4-fluoro-5-methoxyisoindolin-2-yl)-4-oxobutanoate (0.039 g, 60.4948 μmol) in THF (2 mL), EtOH (2 mL) and Water (2 mL) at 20° C. . The reaction mixture was stirred for 2 h at 20° C.

The reaction mixture was adjusted to pH=3 with HCl (1 mol/L). The reaction mixture was evaporated under reduced pressure. The residue was purified on C18 column ACN/H₂O (0.1% FA)(0-42%). The pure fraction was concentrated and dried by lyophilization. There was 4-(6-(3-((2-(3-carboxypropanoyl)-6-methoxyisoindolin-5-yl)oxy)propoxy)-4-fluoro-5-methoxyisoindolin-2-yl)-4-oxobutanoic acid (0.025 g, 42.4753 mol, 70.2131% yield) obtained as a white solid. LCMS: (ESI, m/z): [M+H]⁺=589.210. ¹H NMR (400 MHz, DMSO-d) 6 7.03-6.90 (m, 3H), 4.85-4.70 (m, 4H), 4.59-4.49 (m, 4H), 4.22-4.17 (m, 2H), 4.13 (t, J=6.2 Hz, 2H), 3.78 (s, 3H), 3.74 (s, 3H), 2.61-2.53 (m, 4H), 2.48-2.43 (m, 4H), 2.24-2.16 (m, 2H).

### Example I-3

sodium (S)-4-(5-(3-((2-(3-carboxylatopropanoyl)-6-methoxyisoindolin-5-yl)oxy)propoxy)-6-methoxyisoindolin-2-yl)-2-methyl-4-oxobutanoate

### Step a: methyl (2S)-4-(5-methoxyisoindolin-2-yl)-2-methyl-4-oxo-butanoate

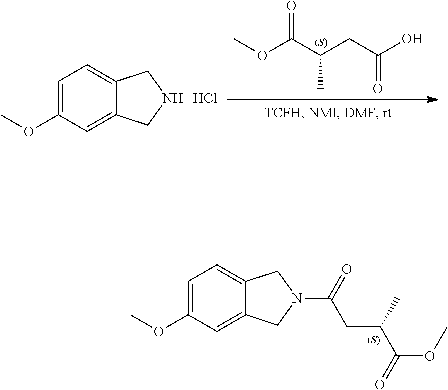

NMI (7.96 g, 96.9507 mmol) was added to a solution of TCFH (8.11 g, 28.9045 mmol) and (S)-4-methoxy-3-methyl-4-oxobutanoic acid (2.81 g, 19.2280 mmol) in DMF (100 mL) at 25° C. The reaction mixture was stirred for 10 min at 20° C. 5-methoxyisoindoline hydrochloride (4.99 g, 26.8785 mmol) was added to the solution at 25° C. The reaction mixture was stirred 2 h at 25° C. The reaction mixture was concentrated and diluted with H₂O (200 mL), extracted with EA (2×200 mL) and washed with brine (100 mL). The organics dried over Na₂SO₄, filtered and evaporated under reduced pressure. The residue was purified on silica gel column EA/n-Hexane (0-55%). The pure fraction was concentrated and dried under vacuo. There was methyl (2S)-4-(5-methoxyisoindolin-2-yl)-2-methyl-4-oxo-butanoate (3.49 g, 12.5850 mmol, 65.4512% yield) obtained as a yellow solid. LCMS: (ESI, m/z): [M+H]⁺=278.130. ¹H NMR (400 MHz, DMSO-d₆) δ 7.23 (t, J=8.0 Hz, 1H), 6.95-6.84 (m, 2H), 4.78 (s, 1H), 4.73 (s, 1H), 4.56 (s, 1H), 4.52 (s, 1H), 3.75 (d, J=1.8 Hz, 3H), 3.60 (s, 3H), 2.91-2.81 (m, 1H), 2.76-2.66 (m, 1H), 2.54-2.51 (m, 0.5H), 2.49-2.45 (m, 0.5H), 1.18-1.14 (m, 3H).

### Step b: methyl (2S)-4-(5-bromo-6-methoxy-isoindolin-2-yl)-2-methyl-4-oxo-butanoate

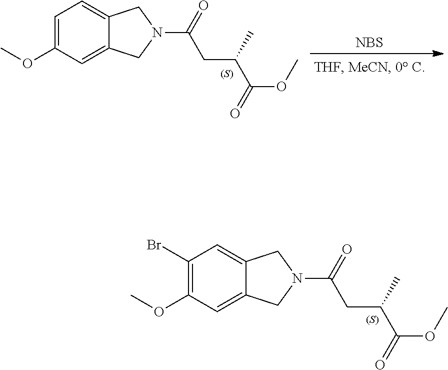

NBS (3.717 g, 20.8839 mmol) was added to a solution of methyl (2S)-4-(5-methoxyisoindolin-2-yl)-2-methyl-4-oxo-butanoate (3.426 g, 12.3542 mmol) in THF (30 mL) and MeCN (30 mL) at 0° C. The reaction mixture was stirred for 3 h at 25° C. The reaction mixture was concentrated and diluted with DCM (200 mL), washed with H₂O (100 mL), KHCO₃ (aq.)(2×100 mL) and brine (100 mL). The organics dried over Na₂SO₄, filtered and evaporated under reduced pressure. The residue was purified on silica gel column EA/n-Hexane (0-60%). The pure fraction was concentrated and dried under vacuo. There was methyl (2S)-4-(5-bromo-6-methoxy-isoindolin-2-yl)-2-methyl-4-oxo-butanoate (3.02 g, 8.4781 mmol, 68.6255% yield) obtained as a yellow solid.

LCMS: (ESI, m/z): [M+H]⁺=356.040. ¹H NMR (400 MHz, DMSO-d₆) δ 7.57 (s, 0.5H), 7.55 (s, 0.5H), 7.14 (s, 0.5H), 7.09 (s, 0.5H), 4.78 (s, 1H), 4.75 (s, 1H), 4.56 (s, 1H), 4.53 (s, 1H), 3.86 (s, 3H), 3.59 (s, 3H), 2.93-2.79 (m, 1H), 2.78-2.64 (m, 1H), 2.49-2.46 (m, 1H), 1.15 (d, J=7.1 Hz, 3H).

### Step c: methyl (2S)-4-[5-methoxy-6-(4,4,5,5-tetramethyl-1,3,2-dioxaborolan-2-yl)isoindolin-2-yl]-2-methyl-4-oxo-butanoate

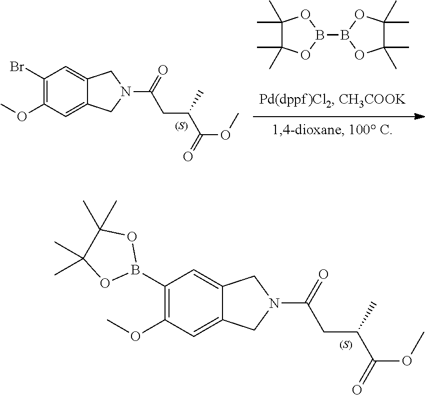

Potassium Acetate (2.81 g, 28.6319 mmol) was added to a mixture of methyl (2S)-4-(5-bromo-6-methoxy-isoindolin-2-yl)-2-methyl-4-oxo-butanoate (3.00 g, 8.4220 mmol), [1,1′-Bis(diphenylphosphino)ferrocene]dichloropalladium(II) (0.74 g, 1.0113 mmol) and 4,4,4′,4′,5,5,5′,5′-Octamethyl-2,2′-bi(1,3,2-dioxaborolane) (3.18 g, 12.5228 mmol) in 1,4-Dioxane (100 mL) at 25° C. The reaction mixture was heated to 100° C. and stirred overnight. The reaction mixture was diluted with EA(200 mL), The precipitate was collected by filtration, washed with EA (1×100 mL). The filtrate was evaporated under reduced pressure. The residue was purified on silica gel column EA/n-Hexane (0-50%). The pure fraction was concentrated and dried under vacuo. There was methyl (2S)-4-[5-methoxy-6-(4,4,5,5-tetramethyl-1,3,2-dioxaborolan-2-yl)isoindolin-2-yl]-2-methyl-4-oxo-butanoate (2.38 g, 5.9017 mmol, 70.0746% yield) obtained as a brown solid. LCMS: (ESI, m/z): [M+H]⁺=404.222. ¹H NMR (400 MHz, DMSO-d₆) δ 7.49 (s, 0.5H), 7.46 (s, 0.5H), 6.97 (s, 0.5H), 6.93 (s, 0.5H), 4.81 (s, 1H), 4.73 (s, 1H), 4.59 (s, 1H), 4.52 (s, 1H), 3.73 (s, 3H), 3.59 (s, 3H), 2.90-2.81 (m, 1H), 2.76-2.65 (m, 1H), 2.54-2.51 (m, 1H), 1.27 (s, 12H), 1.15 (d, J=6.6 Hz, 3H).

### Step d: methyl (2S)-4-(5-hydroxy-6-methoxy-isoindolin-2-yl)-2-methyl-4-oxo-butanoate

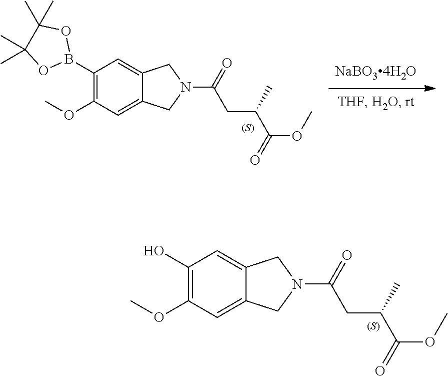

Sodium perborate tetrahydrate (0.80 g, 5.1995 mmol) was added to a solution of methyl (2S)-4-[5-methoxy-6-(4,4,5,5-tetramethyl-1,3,2-dioxaborolan-2-yl)isoindolin-2-yl]-2-methyl-4-oxo-butanoate (1.99 g, 4.9346 mmol) in THF (25 mL) and H₂O (25 mL) at 25° C. The reaction mixture was stirred for 1 h at 25° C. The reaction mixture was concentrated and diluted with H₂O (200 mL), extracted with DCM (3×100 mL) and washed with brine (100 mL). The organics dried over Na₂SO₄, filtered and evaporated under reduced pressure. The residue was purified on C18 column ACN/H₂O (0-40%). The pure fraction was concentrated and dried under vacuo. There was methyl (2S)-4-(5-hydroxy-6-methoxy-isoindolin-2-yl)-2-methyl-4-oxo-butanoate (1.23 g, 4.1935 mmol, 84.9809% yield) obtained as a white solid. LCMS: (ESI, m/z): [M+H]⁺=294.130.

### Step e: methyl (2R)-4-[5-(3-bromopropoxy)-6-methoxy-isoindolin-2-yl]-2-methyl-4-oxo-butanoate

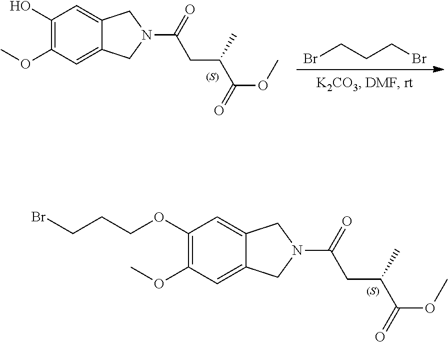

Potassium carbonate (0.462 g, 3.3428 mmol) was added to a solution of methyl (2S)-4-(5-hydroxy-6-methoxy-isoindolin-2-yl)-2-methyl-4-oxo-butanoate (0.311 g, 1.0603 mmol) and 1,3-Dibromopropane (1.158 g, 5.7359 mmol) in DMF (20 mL) at 25° C. The reaction mixture was stirred for 3 h at 25° C. The reaction mixture was purified on C18 column ACN/H₂O (0-45%). The pure fraction was concentrated and dried under vacuo. There was methyl (2R)-4-[5-(3-bromopropoxy)-6-methoxy-isoindolin-2-yl]-2-methyl-4-oxo-butanoate (0.314 g, 757.9234 mol, 71.4822% yield) obtained as a colorless oil. LCMS: (ESI, m/z): [M+H]⁺=414.080.

### Step f: methyl (S)-4-(5-(3-((2-(4-ethoxy-4-oxobutanoyl)-6-methoxyisoindolin-5-yl)oxy)propoxy)-6-methoxyisoindolin-2-yl)-2-methyl-4-oxobutanoate

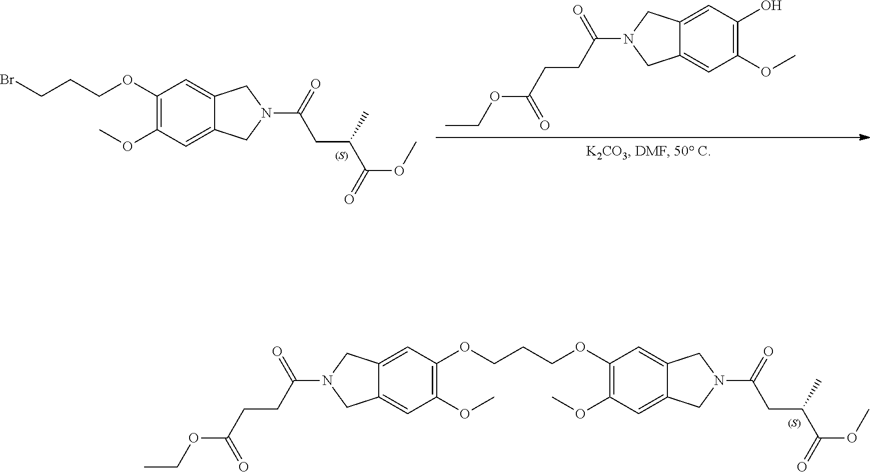

Potassium carbonate (0.106 g, 766.9738 mol) was added to a solution of methyl (2R)-4-[5-(3-bromopropoxy)-6-methoxy-isoindolin-2-yl]-2-methyl-4-oxo-butanoate (0.110 g, 265.5146 mol) and ethyl 4-(5-hydroxy-6-methoxyisoindolin-2-yl)-4-oxobutanoate (0.075 g, 255.6987 mol) in DMF (5 mL) at 25° C. The reaction mixture was heated to 50° C. and stirred overnight. The reaction mixture was purified on C18 column ACN/H₂O(0-40%). The pure fraction was concentrated and dried under vacuo. There was methyl (S)-4-(5-(3-((2-(4-ethoxy-4-oxobutanoyl)-6-methoxyisoindolin-5-yl)oxy)propoxy)-6-methoxyisoindolin-2-yl)-2-methyl-4-oxobutanoate (0.09 g, 143.6111 mol, 54.0879% yield ) obtained as a yellow oil. LCMS: (ESI, m/z): [M+H]⁺=627.280.

### Step g: sodium (S)-4-(5-(3-((2-(3-carboxylatopropanoyl)-6-methoxyisoindolin-5-yl)oxy)propoxy)-6-methoxyisoindolin-2-yl)-2-methyl-4-oxobutanoate

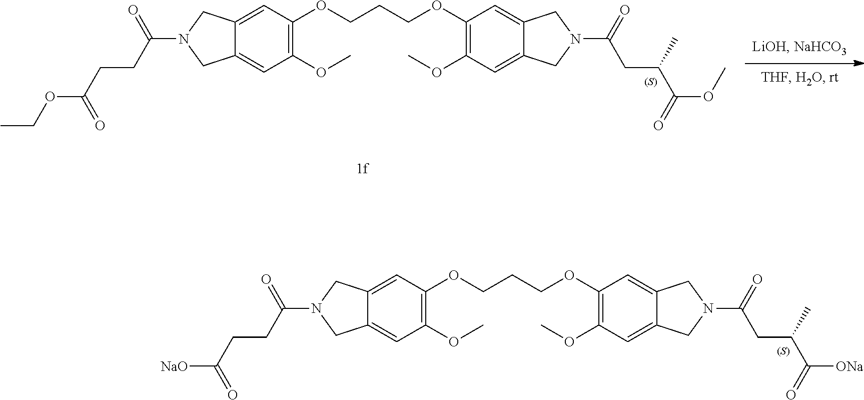

LiOH (0.011 g, 459.3228 mol) was added to a mixture of methyl (S)-4-(5-(3-((2-(4-ethoxy-4-oxobutanoyl)-6-methoxyisoindolin-5-yl)oxy)propoxy)-6-methoxyisoindolin-2-yl)-2-methyl-4-oxobutanoate (0.089 g, 142.0155 μmol) in Water (2 mL) and THF (2 mL) at 25° C. The reaction mixture was stirred for 2 h at 25° C. The reaction mixture was adjusted to pH=3 with HCl (1 mol/L). The mixture was purified on C18 column ACN/H₂O (0-30%).

The pure fraction was concentrated and dried under vacuo and dried by lyophilization to obtain the residue. NaHCO₃ (0.012 g, 142.8459 mol) was added to a mixture of the residue in Water (5 mL) and ACN (2 mL) at 25° C. The reaction mixture was stirred for 1 h at 25° C. The mixture was dried by lyophilization. There was sodium (S)-4-(5-(3-((2-(3-carboxylatopropanoyl)-6-methoxyisoindolin-5-yl)oxy)propoxy)-6-methoxyisoindolin-2-yl)-2-methyl-4-oxobutanoate (0.047 g, 74.7721 mol, 52.6507% yield ) obtained as a grey solid. LCMS: (ESI, m/z): [M+H]⁺=585.240. ¹H NMR (400 MHz, DMSO-d₆) δ 7.00-6.90 (m, 4H), 4.91-4.61 (m, 4H), 4.49 (d, J=9.1 Hz, 4H), 4.15-4.05 (m, 4H), 3.74 (s, 6H), 2.46-2.37 (m, 3H), 2.72-2.71 (m, 1H), 2.20-2.10 (m, 4H), 2.08-1.99 (m, 1H), 1.00 (d, J=7.0 Hz, 3H).

### Example I-4

### 4-(5-(3-((2-(3-carboxypropanoyl)-4-fluoro-6-methoxyisoindolin-5-yl)oxy)propoxy)-6-methoxyisoindolin-2-yl)-4-oxobutanoic acid

### Step a: ethyl 4-(5-(3-((2-(4-ethoxy-4-oxobutanoyl)-4-fluoro-6-methoxyisoindolin-5-yl)oxy)propoxy)-6-methoxyisoindolin-2-yl)-4-oxobutanoate

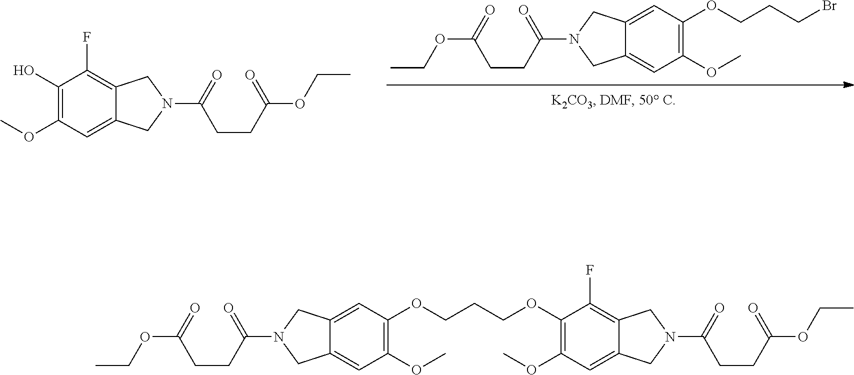

Potassium carbonate (0.111 g, 803.1518 mol) was added to a solution of ethyl 4-(4-fluoro-5-hydroxy-6-methoxyisoindolin-2-yl)-4-oxobutanoate (0.068 g, 218.4354 mol) and ethyl 4-(5-(3-bromopropoxy)-6-methoxyisoindolin-2-yl)-4-oxobutanoate (0.109 g, 263.1008 mol) in DMF (4 mL) at 25° C. under N₂ atmosphere. The reaction mixture was stirred for 3 h at 50° C. The reaction mixture was quenched with adding of water (50 mL) at 25° C., washed with EA (3×50 mL) and brine (50 mL). The organics dried over Na₂SO₄, filtered and evaporated under reduced pressure obtained as a brown oil. The residue was purified on on C18 column ACN/H₂O (0.1% FA)(0-60%). The pure fraction was concentrated and dried by lyophilization. There was ethyl 4-(5-(3-((2-(4-ethoxy-4-oxobutanoyl)-4-fluoro-6-methoxyisoindolin-5-yl)oxy)propoxy)-6-methoxyisoindolin-2-yl)-4-oxobutanoate (0.014 g, 21.7161 mol, 9.9417% yield) obtained as a white solid. ¹H NMR (400 MHz, DMSO-d₆) δ 6.99-6.88 (m, 3H), 4.83 (s, 1H), 4.81 (s, 1H), 4.75 (s, 2H), 4.57 (s, 2H), 4.53 (s, 2H), 4.13 (t, J=6.3 Hz, 4H), 4.05 (q, J=7.0 Hz, 4H), 3.76 (s, 3H), 3.74 (s, 3H), 2.64-2.59 (m, 4H), 2.57-2.53 (m, 4H), 2.12-2.05 (m, 2H), 1.18 (t, J=7.1 Hz, 6H).

### Step b: 4-(5-(3-((2-(3-carboxypropanoyl)-4-fluoro-6-methoxyisoindolin-5-yl)oxy)propoxy)-6-methoxyisoindolin-2-yl)-4-oxobutanoic acid

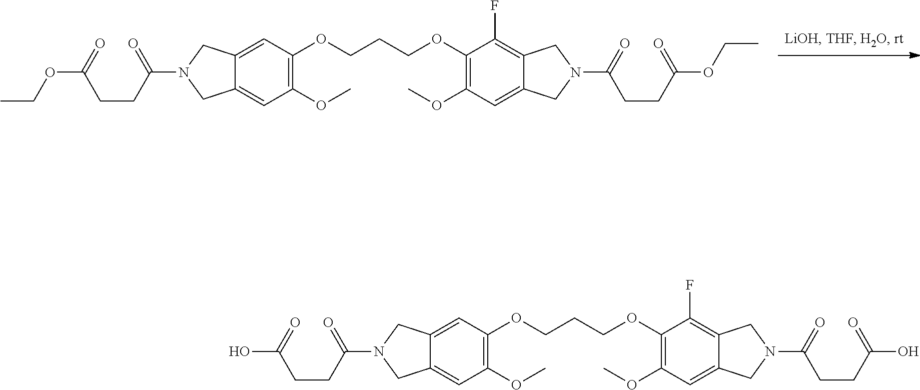 [SMILES: CCOC(=O)CCC(=O)N1Cc2cc(OC)c(OCCCOc3c(OC)cc4c(c3F)CN(C(=O)CCC(=O)OCC)C4)cc2C1 ; COc1cc2c(cc1OCCCOc1c(OC)cc3c(c1F)CN(C(=O)CCC(=O)O)C3)CN(C(=O)CCC(=O)O)C2]

LiOH (0.044 g, 1.8373 mmol) was added to a mixture of ethyl 4-(5-(3-((2-(4-ethoxy-4-oxobutanoyl)-4-fluoro-6-methoxyisoindolin-5-yl)oxy)propoxy)-6-methoxyisoindolin-2-yl)-4-oxobutanoate (0.100 g, 155.1150 μmol) in THF (2 mL) and Water (2 mL) at 25° C. . The reaction mixture was stirred for 2 h at 25° C. The reaction mixture was adjusted to pH=3 with HCl (1 mol/L). The reaction mixture was evaporated under reduced pressure.

The residue was purified on C18 column ACN/H₂O (0.1% FA)(0-50%). The pure fraction was concentrated and dried further in lyophilization. There was 4-(5-(3-((2-(3-carboxypropanoyl)-4-fluoro-6-methoxyisoindolin-5-yl)oxy)propoxy)-6-methoxyisoindolin-2-yl)-4-oxobutanoic acid (0.041 g, 69.6595 mol, 44.9083% yield) obtained as a white solid. LCMS: (ESI, m/z): [M+H]⁺=589.210. ¹H NMR (400 MHz, DMSO-d₆) δ 12.08 (s, 2H), 7.00-6.88 (m, 3H), 4.82 (s, 1H), 4.80 (s, 1H), 4.74 (s, 2H), 4.58 (s, 2H), 4.53 (s, 2H), 4.13 (t, J=6.2 Hz, 4H), 3.77 (s, 3H), 3.74 (s, 3H), 2.62-2.54 (m, 4H), 2.53-2.47 (m, 4H), 2.13-2.03 (m, 2H).

### Example I-5

### 4,4′-((propane-1,3-diylbis(oxy))bis(3-methoxy-5,7-dihydro-6H-pyrrolo[3,4-b]pyridine-2,6-diyl))bis(4-oxobutanoic acid)

### Step a: 5-bromo-2,3-bis(bromomethyl)pyridine

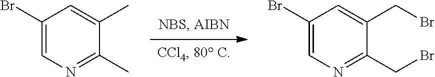

NBS (129.49 g, 727.5373 mmol) was added to the mixture of 5-bromo-2,3-dimethylpyridine (65.61 g, 352.6494 mmol) and AIBN (0.98 g, 5.9681 mmol) in CC14 (1000 mL) at 25° C. The reaction mixture was heated to 80° C. and stirred for 2 h. The reaction mixture was cooled down to 25° C. The reaction mixture was filtered through a short silica column, washed with DCM (3×400 mL). The filtrate was concentrated and dried under vacuo at 35° C. There was 5-bromo-2,3-bis(bromomethyl)pyridine (127.9 g, 185.9871 mmol, 52.7400% yield) obtained as a red oil. LCMS: (ESI, m/z): [M+H]⁺=341.805.

### Step b: 3-bromo-6-trityl-5,7-dihydropyrrolo[3,4-b]pyridine

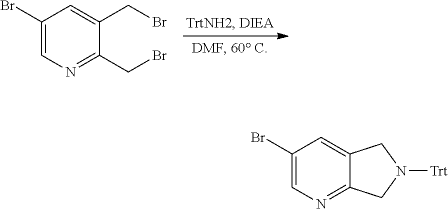

N,N-Diisopropylethylamine (78.2000 g, 605.0646 mmol) was added to a solution of triphenylmethanamine (93.77 g, 361.5657 mmol) and 5-bromo-2,3-bis(bromomethyl)pyridine (125.17 g, 182.0173 mmol) in DMF (800 mL) at 25° C. The reaction mixture was heated to 60° C. and stirred for overnight. The reaction mixture was evaporated under reduced pressure. The reaction mixture was diluted with EA (800 mL), washed with water (500 mL) and brine (300 mL). The organics dried over Na₂SO₄, filtered and evaporated under reduced pressure. The residue was purified on silica gel column EA/n-Hexane (0-10%). The pure fraction was concentrated and dried under vacuo at 35° C. There was 3-bromo-6-trityl-5,7-dihydropyrrolo[3,4-b]pyridine (42.00 g, 95.1601 mmol) obtained as a yellow sem-solid. LCMS: (ESI, m/z): [M+H]⁺=441.089.

### Step c: benzyl 3-bromo-5,7-dihydropyrrolo[3,4-b]pyridine-6-carboxylate

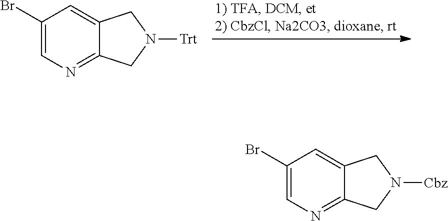

Trifluoroacetic acid (100 mL, 1.3462 mol) was added to a solution of 3-bromo-6-trityl-5,7-dihydropyrrolo[3,4-b]pyridine (20.63 g, 46.7417 mmol) in DCM (100 mL) at 0° C. The reaction mixture was stirred overnight at 25° C. The reaction mixture was evaporated under reduced pressure. The reaction mixture was diluted with HCl (1 M, 150 mL), washed with EA (3×200 mL). The aqueous was neutralized to pH 7-8 by adding of NaOH (4 M) at 0° C. Sodium carbonate (16.45 g, 155.2048 mmol) and 1,4-Dioxane (100 mL) was added to aqueous above. Carbobenzyloxy chloride (16.9680 g, 99.4650 mmol) was added dropwise to the mixture at 0° C. The reaction mixture stirred for 1 h at 25° C. The reaction mixture was extracted with EA (600 mL), washed with water (200 mL) and brine (100 mL). The organics dried over Na₂SO₄, filtered and evaporated under reduced pressure. The residue was purified on silica gel column EA/n-Hexane (0-20%) to remove impurities, and then EA/DCM (10%). The pure fraction was concentrated and dried under vacuo. There was benzyl 3-bromo-5,7-dihydropyrrolo[3,4-b]pyridine-6-carboxylate (8.49 g, 25.4818 mmol, 54.5161% yield) obtained as a yellow solid.

LCMS: (ESI, m/z): [M+H]⁺=333.016. ¹H NMR (400 MHz, DMSO-d₆) δ 8.59 (s, 1H), 8.08 (s, 0.5H), 8.04 (s, 0.5H), 7.45-7.29 (m, 5H), 5.16 (d, J=2.9 Hz, 2H), 4.74 (s, 1H), 4.68 (s, 1H), 4.65 (s, 1H), 4.58 (s, 1H).

### Step d: 3-methoxy-5,7-dihydropyrrolo[3,4-b]pyridine-6-carboxylate

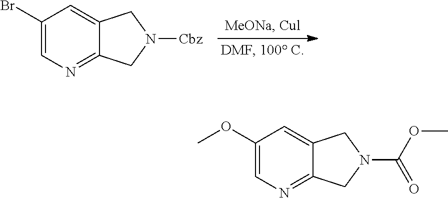

Sodium methanolate (160.63 g, 891.9991 mmol) and Cuprous iodide (8.17 g, 42.8984 mmol) was added to a solution of benzyl 3-bromo-5,7-dihydropyrrolo[3,4-b]pyridine-6-carboxylate (16.21 g, 48.6525 mmol) in DMF (200 mL) at 25° C. The reaction mixture was heated to 100° C. and stirred for 2 h at N₂ atmosphere. The reaction mixture was concentrated and diluted with EA (1000 mL), washed with water (500 mL) and brine (500 mL). The organics dried over Na₂SO₄, filtered and evaporated under reduced pressure. The residue was purified on silica gel column EA/n-Hexane (0-100%). The pure fraction was concentrated and dried under vacuo. There was methyl 3-methoxy-5,7-dihydropyrrolo[3,4-b]pyridine-6-carboxylate (5.16 g, 24.7823 mmol, 50.9373% yield) obtained as a yellow solid. LCMS: (ESI, m/z): [M+H]⁺=209.085. ¹H NMR (400 MHz, DMSO-d₆) δ 8.16 (s, 1H), 7.42 (s, 0.5H), 7.38 (s, 0.5H), 4.64 (s, 1H), 4.61 (s, 1H), 4.52 (s, 1H), 4.50 (s, 1H), 3.82 (s, 3H), 3.68 (s, 3H).

### Step e: methyl 3-methoxy-1-oxido-5,7-dihydropyrrolo[3,4-b]pyridin-1-ium-6-carboxylate

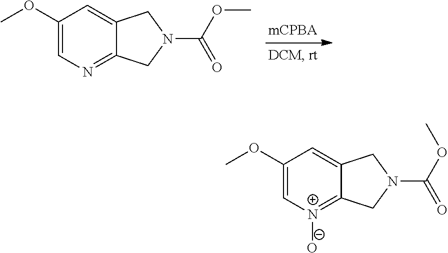

M-Chloroperoxybenzoic acid (7.24 g, 33.5640 mmol) was added to a solution of methyl 3-methoxy-5,7-dihydropyrrolo[3,4-b]pyridine-6-carboxylate (5.07 g, 24.3500 mmol) in DCM (100 mL) at 25° C. The reaction mixture was stirred for 2 h at 25° C. The reaction mixture was diluted with DCM (500 mL), washed with KHCO₃ (aq)(2×200 mL) and brine (100 mL). The organics dried over Na₂SO₄, filtered and evaporated under reduced pressure. The residue was purified on silica gel column MeOH/DCM (0-10%). There was methyl 3-methoxy-1-oxido-5,7-dihydropyrrolo[3,4-b]pyridin-1-ium-6-carboxylate (3.39 g, 15.1196 mmol, 62.0926% yield) obtained as a white solid. LCMS: (ESI, m/z): [M+H]⁺=225.080. ¹H NMR (400 MHz, DMSO-d₆) δ 8.03 (s, 1H), 7.10 (s, 0.5H), 7.07 (s, 0.5H), 4.68 (s, 1H), 4.65 (s, 1H), 4.55 (d, J=8.2 Hz, 1H), 4.53 (s, 1H), 3.81 (s, 3H), 3.67 (s, 3H).

### Step f: methyl 2-chloro-3-methoxy-5,7-dihydropyrrolo[3,4-b]pyridine-6-carboxylate

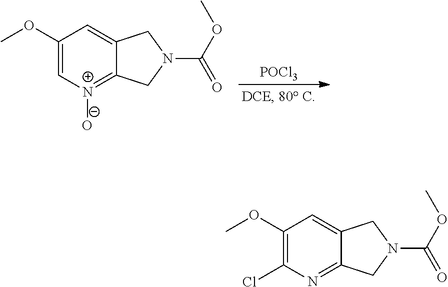

Phosphorus oxychloride (32.90 g, 214.5668 mmol) was added to a solution of methyl 3-methoxy-1-oxido-5,7-dihydropyrrolo[3,4-b]pyridin-1-ium-6-carboxylate (6.31 g, 23.1065 mmol) in DCE (100 mL) at 25° C. The reaction mixture was heated to 80° C. and stirred for 2 h. The reaction mixture was evaporated under reduced pressure. The mixture was adjusted to pH=8 with saturated NaHCO₃ (aq). The reaction mixture was concentrated and diluted with DCM (1000 mL), washed with water (500 mL) and brine (500 mL). The organics dried over Na₂SO₄, filtered and evaporated under reduced pressure. The residue was purified on silica gel column EA/DCM (0-50%). The pure fraction was concentrated and dried under vacuo. There was methyl 2-chloro-3-methoxy-5,7-dihydropyrrolo[3,4-b]pyridine-6-carboxylate (6.30 g, 25.9624 mmol, 112.3596% yield) obtained as a off-white solid. LCMS: (ESI, m/z): [M+H]⁺=243.046. ¹H NMR (400 MHz, CDCl₃-d) 6 7.08 (s, 0.5H), 7.03 (s, 0.5H), 4.67 (s, 1H), 4.61 (d, J=3.2 Hz, 2H), 4.55 (s, 1H), 3.86 (s, 3H), 3.73 (s, 3H).

### Step g: 2-chloro-3-methoxy-6,7-dihydro-5H-pyrrolo[3,4-b]pyridine

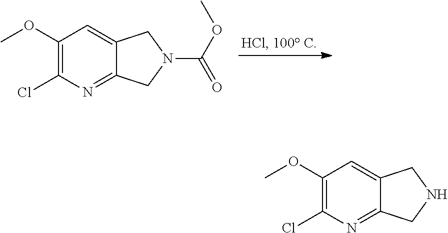 [SMILES: COC(=O)N1Cc2cc(OC)c(Cl)nc2C1 ; COc1cc2c(nc1Cl)CNC2]

To a solution of methyl 2-chloro-3-methoxy-5,7-dihydropyrrolo[3,4-b]pyridine-6-carboxylate (4.22 g, 17.3907 mmol) in hydrogen chloride (80 mL) at 25° C. The reaction mixture was heated to 100° C. and stirred overnight. The reaction mixture was evaporated under reduced pressure. The residue was dissolved in water (10 mL) and adjusted to pH=8 with NaOH (aq.)(4 M). The residue was purified on silica gel column MeCN/water (0-50%). The pure fraction was concentrated and dried under vacuo. There was 2-chloro-3-methoxy-6,7-dihydro-5H-pyrrolo[3,4-b]pyridine (1.688 g, 9.1430 mmol, 52.5739% yield) obtained as a yellow semi-solid. LCMS: (ESI, m/z): [M+H]⁺=185.040.

### Step h: 2-chloro-3-methoxy-6-(4-methoxybenzyl)-6,7-dihydro-5H-pyrrolo[3,4-b]pyridine

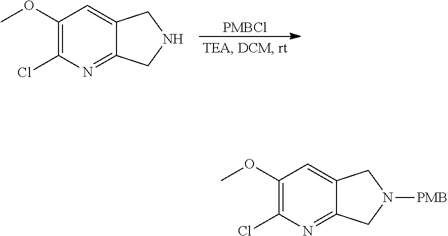

4-Methoxybenzylchloride (1.507 g, 9.6227 mmol) was added to a solution of 2-chloro-3-methoxy-6,7-dihydro-5H-pyrrolo[3,4-b]pyridine (1.781 g, 9.6467 mmol) and TEA (1.985 g, 19.6167 mmol) in DCM (40 mL) at 25° C. The reaction mixture was stirred overnight at 25° C. The reaction mixture was concentrated and diluted with DCM (600 mL), washed with water (100 mL) and brine (100 mL). The organics dried over Na₂SO₄, filtered and evaporated under reduced pressure. The residue was purified on silica gel column EA/DCM (0-50%). The pure fraction was concentrated and dried under vacuo. There was 2-chloro-3-methoxy-6-(4-methoxybenzyl)-6,7-dihydro-5H-pyrrolo[3,4-b]pyridine (1.808 g, 5.9323 mmol, 61.4957% yield) obtained as a yellow oil. LCMS: (ESI, m/z): [M+H]⁺=305.098. ¹H NMR (400 MHz, DMSO-d₆) δ 7.51 (s, 1H), 7.28 (d, J=8.3 Hz, 2H), 6.91 (d, J=8.3 Hz, 2H), 3.94 (s, 1H), 3.89-3.78 (m, 7H), 3.77-3.73 (m, 4H).

### Step i: 3-methoxy-6-(4-methoxybenzyl)-2-(3-((tetrahydro-2H-pyran-2-yl)oxy)propoxy)-6,7-dihydro-5H-pyrrolo[3,4-b]pyridine

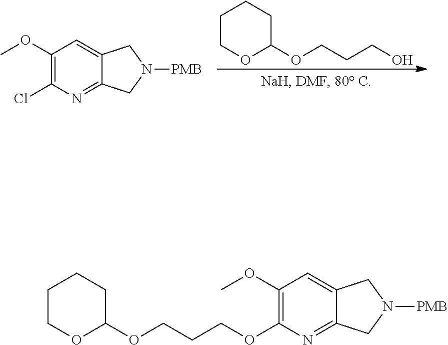

NaH (1.25 g, 31.2530 mmol) was added to a solution of 3-((tetrahydro-2H-pyran-2-yl)oxy)propan-1-ol (4.73 g, 29.5237 mmol) in DMF (30 mL) at 0° C. The reaction mixture was stirred for 30 min at 25° C. 2-chloro-3-methoxy-6-(4-methoxybenzyl)-6,7-dihydro-5H-pyrrolo[3,4-b]pyridine (1.81 g, 5.9389 mmol) was added to the mixture at 25° C. The reaction mixture was heated to 80° C. and stirred for 2 h. The reaction mixture was concentrated and diluted with EA (600 mL), washed with water (200 mL) and brine (100 mL). The organics dried over Na₂SO₄, filtered and evaporated under reduced pressure. The residue was purified on silica gel column MeOH/DCM (0-10%). The pure fraction was concentrated and dried under vacuo. There was 3-methoxy-6-(4-methoxybenzyl)-2-(3-((tetrahydro-2H-pyran-2-yl)oxy)propoxy)-6,7-dihydro-5H-pyrrolo[3,4-b]pyridine (3.114 g, 5.8135 mmol, 97.8886% yield) obtained as a yellow oil. LCMS: (ESI, m/z): [M+H]⁺=429.231.

### Step j: 3-((3-methoxy-6-(4-methoxybenzyl)-6,7-dihydro-5H-pyrrolo[3,4-b]pyridin-2-yl)oxy)propan-1-ol

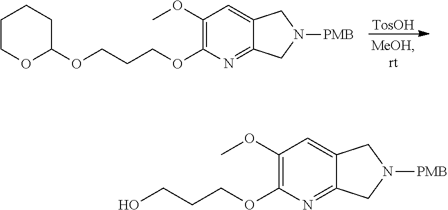

4-methylbenzenesulfonic acid (0.331 g, 1.2199 mmol) was added to a solution of 3-methoxy-6-(4-methoxybenzyl)-2-(3-((tetrahydro-2H-pyran-2-yl)oxy)propoxy)-6,7-dihydro-5H-pyrrolo[3,4-b]pyridine (0.502 g, 1.1715 mmol) in MeOH (10 mL) at 25° C. The reaction mixture was stirred at 25° C. for 1 h. The mixture was adjusted to pH=8 with saturated NaHCO₃ (aq). The reaction mixture was concentrated and diluted with DCM (500 mL), washed with water (200 mL) and brine (100 mL). The organics dried over Na₂SO₄, filtered and evaporated under reduced pressure. The residue was purified on silica gel column MeOH/DCM (0-10%). The pure fraction was concentrated and dried under vacuo. There was 3-((3-methoxy-6-(4-methoxybenzyl)-6,7-dihydro-5H-pyrrolo[3,4-b]pyridin-2-yl)oxy)propan-1-ol (0.180 g, 522.6422 mol, 44.6141% yield) obtained as a yellow oil.

LCMS: (ESI, m/z): [M+H]⁺=345.174. ¹H NMR (400 MHz, DMSO-d₆) δ 7.28 (d, J=8.3 Hz, 2H), 7.20 (s, 1H), 6.90 (d, J=8.3 Hz, 2H), 4.50 (t, J=4.7 Hz, 1H), 4.26 (t, J=6.5 Hz, 2H), 3.80-3.75 (m, 4H), 3.74 (d, J=3.7 Hz, 3H), 3.73 (s, 3H), 3.72 (s, 2H), 3.58-3.47 (m, 2H), 1.90-1.79 (m, 2H).

### Step k: 1,3-bis((3-methoxy-6-(4-methoxybenzyl)-6,7-dihydro-5H-pyrrolo[3,4-b]pyridin-2-yl)oxy)propane

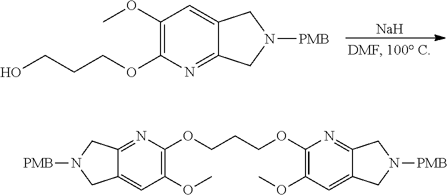

NaH (0.142 g, 3.5503 mmol) was added to a solution of 3-((3-methoxy-6-(4-methoxybenzyl)-6,7-dihydro-5H-pyrrolo[3,4-b]pyridin-2-yl)oxy)propan-1-ol (0.176 g, 511.0277 mol) in DMF (5 mL) at 0° C. The mixture was allowed to warm to 25° C. and stirred for 40 min. 2-chloro-3-methoxy-6-[(4-methoxyphenyl)methyl]-5,7-dihydropyrrolo[3,4-b]pyridine (0.302 g, 990.9079 mol) was added to the mixture. The reaction mixture was heated to 100° C. and stirred for 1 h. The reaction mixture was quenched with adding of water (10 mL) at 0° C. The resulting mixture was extracted with EA (2×300 mL), washed with water (100 mL) and brine (100 mL). The organics dried over Na₂SO₄, filtered and evaporated under reduced pressure. The residue was purified on silica gel column MeOH/DCM (0-10%). The pure fraction was concentrated and dried under vacuo. There was 1,3-bis((3-methoxy-6-(4-methoxybenzyl)-6,7-dihydro-5H-pyrrolo[3,4-b]pyridin-2-yl)oxy)propane (0.234 g, 381.9076 μmol, 74.7332% yield) obtained as a yellow oil. LCMS: (ESI, m/z): [M+H]⁺=613.295.

### Step l: 1,3-bis((3-methoxy-6,7-dihydro-5H-pyrrolo[3,4-b]pyridin-2-yl)oxy)propane

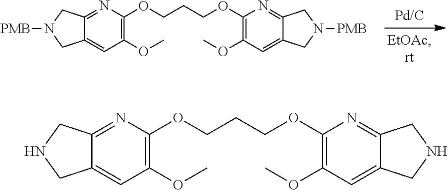

Pd/C (0.145 g, 136.2526 mol) was added to a solution of 1,3-bis((3-methoxy-6-(4-methoxybenzyl)-6,7-dihydro-5H-pyrrolo[3,4-b]pyridin-2-yl)oxy)propane (0.093 g, 151.7837 mol) in Ethyl acetate (10 mL) at 25° C. The reaction mixture was stirred overnight at 25° C. under H₂ atmosphere. The resulting mixture was filtered, the filter cake was washed with DCM/MeOH=5:1 (3×50 mL). The filtrate was concentrated under reduced pressure.

There was 1,3-bis((3-methoxy-6,7-dihydro-5H-pyrrolo[3,4-b]pyridin-2-yl)oxy)propane (0.039 g, 104.7213 mol, 68.9938% yield) obtained as a red solid. LCMS: (ESI, m/z): [M+H]⁺=373.180.

### Step m: dimethyl 4,4′-((propane-1,3-diylbis(oxy))bis(3-methoxy-5,7-dihydro-6H-pyrrolo[3,4-b]pyridine-2,6-diyl))bis(4-oxobutanoate)

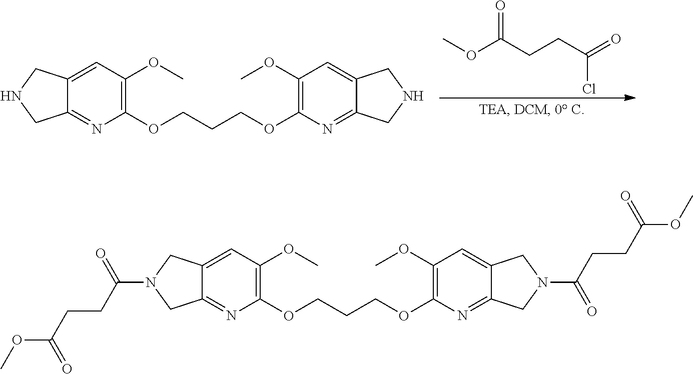

Methyl 4-chloro-4-oxobutanoate (0.061 g, 405.1541 mol) was added to a solution of 1,3-bis((3-methoxy-6,7-dihydro-5H-pyrrolo[3,4-b]pyridin-2-yl)oxy)propane (0.037 g, 99.3510 mol) and TEA (0.076 g, 751.0668 μmol) in DCM (3 mL) at 0° C. under N₂ atmosphere. The reaction mixture was stirred for 1 h at 25° C. The reaction mixture was quenched with adding of water (50 mL) at 25° C. The reaction mixture was extracted with EA (3×50 mL), washed with brine (50 mL). The organics dried over Na₂SO₄, filtered and evaporated under reduced pressure. The residue was purified on C18 column ACN/H₂O (0.1% FA)(0-40%). The pure fraction was concentrated and dried under vacuo. There was dimethyl 4,4′-((propane-1,3-diylbis(oxy))bis(3-methoxy-5,7-dihydro-6H-pyrrolo[3,4-b]pyridine-2,6-diyl))bis(4-oxobutanoate) (0.017 g, 28.3043 mol, 28.4892% yield) obtained as a white solid. LCMS: (ESI, m/z): [M+H]⁺=601.240. ¹H NMR (400 MHz, DMSO-d₆) δ 7.37-7.31 (m, 2H), 4.76 (s, 2H), 4.64 (s, 2H), 4.52 (s, 2H), 4.44-4.35 (m, 6H), 3.78 (s, 6H), 3.59 (s, 6H), 2.65-2.60 (m, 4H), 2.59-2.55 (m, 4H), 2.22-2.16 (m, 2H).

### Step n: 4,4′-((propane-1,3-diylbis(oxy))bis(3-methoxy-5,7-dihydro-6H-pyrrolo[3,4-b]pyridine-2,6-diy))bis(4-oxobutanoic acid)

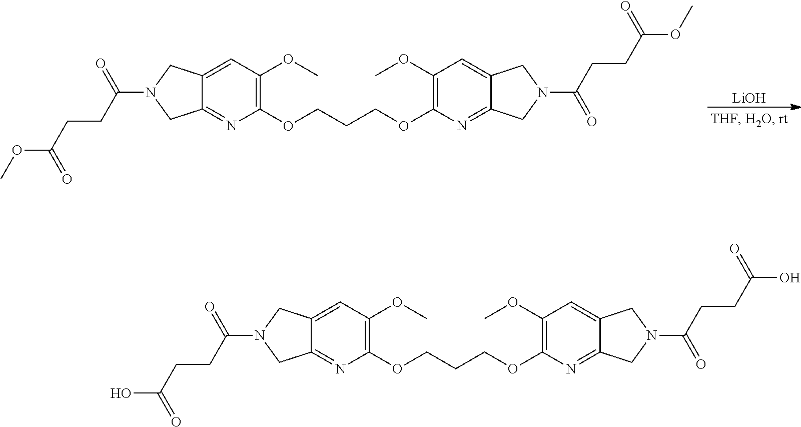

LiOH (0.049 g, 2.0461 mmol) was added to a solution of dimethyl 4,4′-((propane-1,3-diylbis(oxy))bis(3-methoxy-5,7-dihydro-6H-pyrrolo[3,4-b]pyridine-2,6-diyl))bis(4-oxobutanoate) (0.012 g, 19.9795 mol) in THF (2 mL) and Water (2 mL) at 25° C. . The reaction mixture was stirred for 2 h at 25° C. The reaction mixture was adjusted to pH=3 with HCl (1 mol/L). The reaction mixture was evaporated under reduced pressure. The residue was purified on C18 column ACN/H₂O (0-40%). The pure fraction was concentrated and dried under vacuo. There was 4,4′-((propane-1,3-diylbis(oxy))bis(3-methoxy-5,7-dihydro-6H-pyrrolo[3,4-b]pyridine-2,6-diyl))bis(4-oxobutanoic acid) (7 mg, 12.2257 mol, 61.1914% yield) obtained as a white solid. LCMS: (ESI, m/z): [M−H]—=571.210. ¹H NMR (400 MHz, DMSO-d₆) δ 7.32-7.27 (m, 2H), 4.76 (s, 2H), 4.68 (s, 2H), 4.56 (s, 2H), 4.49-4.40 (m, 6H), 3.78-3.71 (m, 6H), 2.44-2.40 (m, 4H), 2.38-2.35 (m, 4H), 2.18-2.12 (m, 2H).

### Example I-6

### 4-(5-(3-((2-(4-ethoxy-4-oxobutanoyl)-6-methoxyisoindolin-5-yl)oxy)propoxy)-6-methoxyisoindolin-2-yl)-4-oxobutanoic acid

### Step a: tert-butyl 5-methoxyisoindoline-2-carboxylate

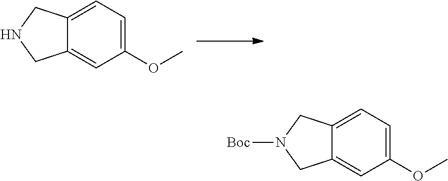 [SMILES: COc1ccc2c(c1)CNC2 ; COc1ccc2c(c1)CN(C(=O)OC(C)(C)C)C2]

TEA (33.12 g, 327.3070 mmol) was added to a solution of 5-Methoxyisoindoline hydrochloride (20.01 g, 107.7833 mmol) and Di-tert-butyl dicarbonate (35.28 g, 161.6523 mmol) in DCM (500 mL) at 20° C. The reaction mixture was stirred overnight at 20° C. The reaction mixture was evaporated under reduced pressure. The residue was purified on silica gel column EA/n-Hexane (0-50%). The pure fraction was concentrated and dried under vacuo. There was tert-butyl 5-methoxyisoindoline-2-carboxylate (30.66 g, 104.5347 mmol, 96.9859% yield) obtained as a white solid. LCMS: (ESI, m/z): [M+H-tBu+ACN]⁺=235.136. ¹H NMR (400 MHz, DMSO-d₆) δ 7.24-7.18 (m, 1H), 6.90 (d, J=4.5 Hz, 1H), 6.85 (s, 0.5H), 6.83 (s, 0.5H), 4.58-4.44 (m, 4H), 3.74 (s, 3H), 1.45 (s, 9H).

### Step b: tert-butyl 5-bromo-6-methoxy-isoindoline-2-carboxylate

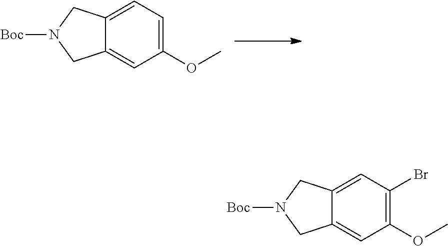 [SMILES: COc1ccc2c(c1)CN(C(=O)OC(C)(C)C)C2 ; COc1cc2c(cc1Br)CN(C(=O)OC(C)(C)C)C2]

NBS (43.64 g, 245.1906 mmol) was added to a solution of tert-butyl 5-methoxyisoindoline-2-carboxylate (30.40 g, 121.9390 mmol) in Tetrahydrofuran (300 mL) and Acetonitrile (300 mL) at 20° C. The reaction mixture was stirred overnight at 20° C. The reaction mixture was evaporated under reduced pressure. The reaction mixture was concentrated and diluted with EA (1000 mL), washed with NaHCO₃ (aq.)(3×200 mL) and brine (300 mL). The organics dried over Na₂SO₄, filtered and evaporated under reduced pressure. The residue was purified on silica gel column EA/n-Hexane (0-50%). The pure fraction was concentrated and dried under vacuo. There was tert-butyl 5-bromo-6-methoxy-isoindoline-2-carboxylate (17.61 g, 53.6562 mmol, 44.0025% yield) obtained as a yellow solid. LCMS: (ESI, m/z): [M+H-tBu+ACN]⁺=313.047. ¹H NMR (400 MHz, DMSO-d₆) δ 7.54 (d, J=5.3 Hz, 1H), 7.11 (d, J=4.2 Hz, 1H), 4.51 (t, J=9.8 Hz, 4H), 3.82 (d, J=3.7 Hz, 3H), 1.45 (s, 9H).

### Step c: tert-butyl 5-methoxy-6-(4,4,5,5-tetramethyl-1,3,2-dioxaborolan-2-yl)isoindoline-2-carboxylate

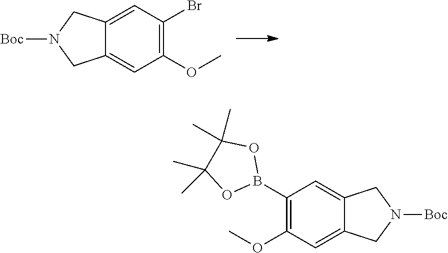 [SMILES: COc1cc2c(cc1Br)CN(C(=O)OC(C)(C)C)C2 ; COc1cc2c(cc1B1OC(C)(C)C(C)(C)O1)CN(C(=O)OC(C)(C)C)C2]

[1,1′-Bis(diphenylphosphino)ferrocene]dichloropalladium(II) (2.10 g, 2.8700 mmol), 4,4,4′,4′,5,5,5′,5′-Octamethyl-2,2′-bi(1,3,2-dioxaborolane) (11.71 g, 46.1137 mmol) and Potassium Acetate (9.76 g, 99.4475 mmol) was added to a solution of tert-butyl 5-bromo-6-methoxy-isoindoline-2-carboxylate (10.04 g, 30.5910 mmol) in 1,4-Dioxane (200 mL) at 20° C. The reaction mixture was heated to 100° C. and stirred overnight under N₂ atmosphere. The reaction mixture was concentrated and diluted with EA (500 mL), washed with water (200 mL) and brine (200 mL). The organics dried over Na₂SO₄, filtered and evaporated under reduced pressure. The residue was purified on silica gel column EA/n-Hexane (0-50%). The pure fraction was concentrated and dried under vacuo. There was tert-butyl 5-methoxy-6-(4,4,5,5-tetramethyl-1,3,2-dioxaborolan-2-yl)isoindoline-2-carboxylate (11.00 g, 29.3126 mmol, 95.8208% yield) obtained as a oil. LCMS: (ESI, m/z): [M+H-tBu+ACN]⁺=361.222.

### Step d: tert-butyl 5-hydroxy-6-methoxy-isoindoline-2-carboxylate

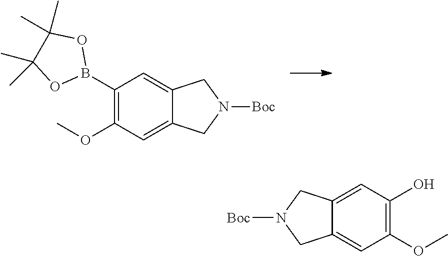 [SMILES: COc1cc2c(cc1B1OC(C)(C)C(C)(C)O1)CN(C(=O)OC(C)(C)C)C2 ; COc1cc2c(cc1O)CN(C(=O)OC(C)(C)C)C2]

Sodium perborate tetrahydrate (6.28 g, 40.8164 mmol) was added to a solution of tert-butyl 5-methoxy-6-(4,4,5,5-tetramethyl-1,3,2-dioxaborolan-2-yl)isoindoline-2-carboxylate (9.75 g, 25.9816 mmol) in Tetrahydrofura (150 mL) and Water (150 mL) at 20° C. The reaction mixture was stirred for 2 h at 20° C. The reaction mixture was concentrated and diluted with EA (600 mL), washed with water (300 mL) and brine (300 mL). The organics dried over Na₂SO₄, filtered and evaporated under reduced pressure. The residue was purified on silica gel column EA/DCM (0-100%). The pure fraction was concentrated and dried under vacuo. There was tert-butyl 5-hydroxy-6-methoxy-isoindoline-2-carboxylate (6.58 g, 24.8017 mmol, 95.4589% yield) obtained as a yellow oil. LCMS: (ESI, m/z): [M+H-tBu+ACN]⁺=251.131. ¹H NMR (400 MHz, DMSO-d₆) δ 8.92 (s, 1H), 6.88 (d, J=5.8 Hz, 1H), 6.70 (d, J=2.3 Hz, 1H), 4.44 (t, J=9.6 Hz, 4H), 3.74 (d, J=3.4 Hz, 3H), 1.44 (s, 9H).

### Step e: tert-butyl 5-(3-((2-(4-ethoxy-4-oxobutanoyl)-6-methoxyisoindolin-5-yl)oxy)propoxy)-6-methoxyisoindoline-2-carboxylate

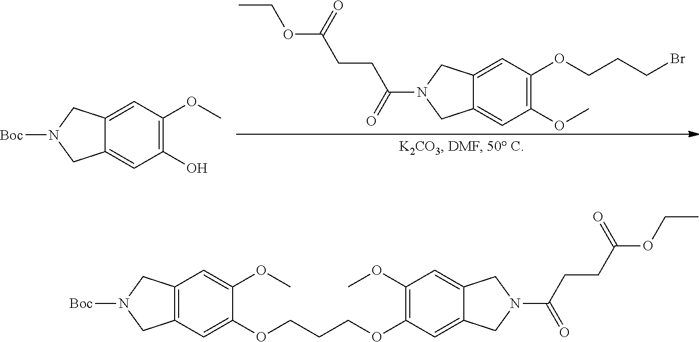 [SMILES: COc1cc2c(cc1O)CN(C(=O)OC(C)(C)C)C2 ; CCOC(=O)CCC(=O)N1Cc2cc(OC)c(OCCCBr)cc2C1 ; CCOC(=O)CCC(=O)N1Cc2cc(OC)c(OCCCOc3cc4c(cc3OC)CN(C(=O)OC(C)(C)C)C4)cc2C1]

Potassium carbonate (0.375 g, 2.7134 mmol) was added to a solution of ethyl 4-(5-(3-bromopropoxy)-6-methoxyisoindolin-2-yl)-4-oxobutanoate (0.364 g, 878.6118 mol) and tert-butyl 5-hydroxy-6-methoxyisoindoline-2-carboxylate (0.264 g, 995.0841 mol) in DMF (8 mL) at 20° C. under N₂ atmosphere. The reaction mixture was heated to 50° C. and stirred overnight. The mixture was purified on C18 column ACN/H₂O (0.1% FA)(0-45%). The pure fraction was concentrated and dried under vacuo. There was tert-butyl 5-(3-((2-(4-ethoxy-4-oxobutanoyl)-6-methoxyisoindolin-5-yl)oxy)propoxy)-6-methoxyisoindoline-2-carboxylate (0.498 g, 831.8270 mol, 94.6751% yield) obtained as a white solid. LCMS: (ESI, m/z): [M+H-tBu+ACN]⁺=584.290. ¹H NMR (400 MHz, DMSO-d₆) δ 7.06-6.90 (m, 4H), 4.74 (s, 1H), 4.72 (s, 1H), 4.60-4.41 (m, 6H), 4.19-3.99 (m, 6H), 3.81-3.69 (m, 6H), 2.65-2.53 (m, 4H), 2.21-2.10 (m, 2H), 1.45 (s, 9H), 1.18 (t, J=8.0 Hz, 3H).

### Step f: 4-(5-(3-((2-(4-ethoxy-4-oxobutanoyl)-6-methoxyisoindolin-5-yl)oxy)propoxy)-6-methoxqisoindolin-2-yl)-4-oxobutanoic acid

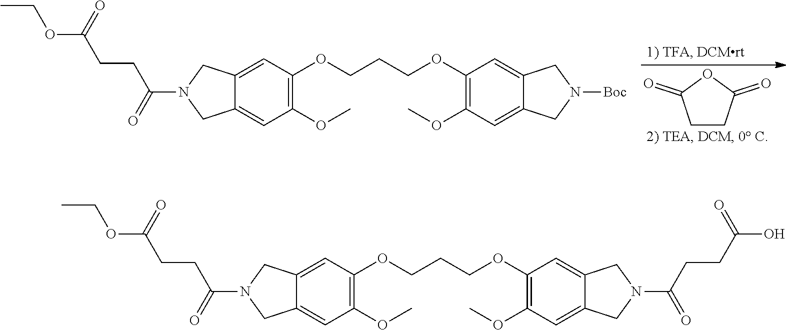

Trifluoroacetic acid (1 mL) was added to a solution of tert-butyl 5-[3-[2-(4-ethoxy-4-oxo-butanoyl)-6-methoxy-isoindolin-5-yl]oxypropoxy]-6-methoxy-isoindoline-2-carboxylate (0.088 g, 146.9896 mol) in DCM (3 mL) at 20° C. . The reaction mixture was stirred for 1 h at 20° C. The reaction mixture was evaporated under reduced pressure. dihydrofuran-2,5-dione (0.050 g, 499.6373 mol) was added to a solution of the residue and TEA (0.209 g, 2.0654 mmol) in DCM (5 mL) at 0° C. under N₂ atmosphere. The reaction mixture was stirred and warmed up to 20° C. naturally. The reaction mixture was stirred for 3 h at 20° C. under N₂ atmosphere. The reaction mixture was quenched with adding of water (50 mL) at 20° C. and adjusted to pH=3 with HCl (1 mol/L), extracted with EA (3×50 mL) and brine (50 mL). The organics dried over Na₂SO₄, filtered and evaporated under reduced pressure. The residue was purified on C18 column ACN/H₂O (0.1% FA)(0-40%). The pure fraction was concentrated and dried under vacuo. There was 4-(5-(3-((2-(4-ethoxy-4-oxobutanoyl)-6-methoxyisoindolin-5-yl)oxy)propoxy)-6-methoxyisoindolin-2-yl)-4-oxobutanoic acid (0.061 g, 101.8978 mol, 69.3231% yield) obtained as a white solid. LCMS: (ESI, m/z): [M−H]⁻=597.250. ¹H NMR (400 MHz, DMSO-d₆) δ 7.02-6.92 (m, 4H), 4.75 (s, 2H), 4.73 (s, 2H), 4.53 (s, 2H), 4.51 (s, 2H), 4.11 (t, J=6.0 Hz, 4H), 4.05 (q, J=7.1 Hz, 2H), 3.74 (s, 6H), 2.65-2.53 (m, 6H), 2.52-2.48 (m, 2H), 2.20-2.11 (m, 2H), 1.18 (t, J=7.1 Hz, 3H).

### Example I-7

### 4,4′-(((methylazanediyl)bis(methylene))bis(6-methoxyisoindoline-5,2-diyl))bis(4-oxobutanoic acid)

### Step a: ethyl 4-(5-methoxy-6-vinyl-isoindolin-2-yl)-4-oxo-butanoate

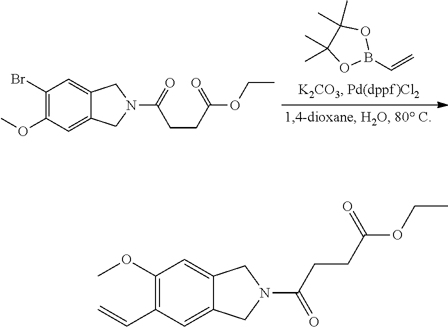

[1,1′-Bis(diphenylphosphino)ferrocene]dichloropalladium(II) (0.223 g, 304.7682 mol) and Potassium carbonate (2.271 g, 16.4321 mmol) was added to a solution of 2-ethenyl-4,4,5,5-tetramethyl-1,3,2-dioxaborolane (1.2349 g, 8.0180 mmol) and ethyl 4-(5-bromo-6-methoxyisoindolin-2-yl)-4-oxobutanoate (1.812 g, 5.0869 mmol) in 1,4-Dioxane (18 mL) and Water (3 mL) at 20° C. The reaction mixture was stirred overnight at 80° C. under nitrogen atmosphere. The reaction mixture was concentrated and diluted with EA (100 mL), washed with NaHCO₃ (aq.)(2×50 mL) and brine (50 mL). The organics dried over Na₂SO₄, filtered and evaporated under reduced pressure. The residue was purified on silica gel column EA/n-Hexane (0-70%). The pure fraction was concentrated and dried under vacuo. There was ethyl 4-(5-methoxy-6-vinyl-isoindolin-2-yl)-4-oxo-butanoate (1.371 g, 4.5195 mmol, 88.8463% yield) obtained as a red solid. LCMS: (ESI, m/z): [M+H]⁺=304.147. ¹H NMR (400 MHz, DMSO-d₆) δ 7.50 (s, 0.5H), 7.48 (s, 0.5H), 7.05-6.90 (m, 2H), 5.80-5.72 (m, 1H), 5.24 (d, J=11.2 Hz, 1H), 4.82 (s, 1H), 4.77 (s, 1H), 4.59 (s, 1H), 4.55 (s, 1H), 4.06 (q, J=7.0 Hz, 2H), 3.81 (s, 3H), 2.65-2.59 (m, 2H), 2.59-2.53 (m, 2H), 1.18 (t, J=7.1 Hz, 3H).

### Step b: ethyl 4-(5-formyl-6-methoxy-isoindolin-2-yl)-4-oxo-butanoate

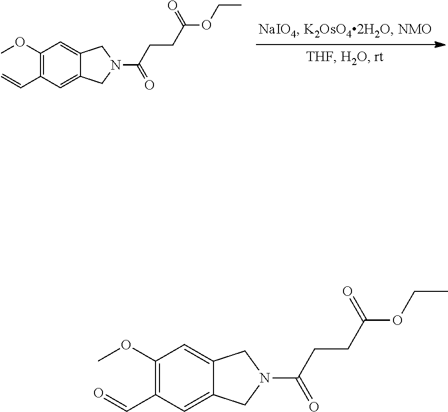 [SMILES: C=Cc1cc2c(cc1OC)CN(C(=O)CCC(=O)OCC)C2 ; CCOC(=O)CCC(=O)N1Cc2cc(C=O)c(OC)cc2C1]

Potassium osmate(VI) dehydrate (0.085 g, 230.6933 mol) in Water (6 mL) was added to a solution of ethyl 4-(5-methoxy-6-vinylisoindolin-2-yl)-4-oxobutanoate (1.295 g, 4.2690 mmol) and 4-Methylmorpholine N-oxide (1.062 g, 9.0656 mmol) in Tetrahydrofuran (26 mL) at 20° C. The reaction mixture was stirred for 2 h at 20° C. Sodium periodate (1.026 g, 4.7968 mmol) was added to the mixture at 20° C. The resulting mixture was stirred overnight at 20° C. The reaction mixture was concentrated and diluted with EA (100 mL), washed with water (50 mL) and brine (50 mL). The organics dried over Na₂SO₄, filtered and evaporated under reduced pressure. The residue was purified on silica gel column EA/n-Hexane (0-100%). The pure fraction was concentrated and dried under vacuo. There was ethyl 4-(5-formyl-6-methoxy-isoindolin-2-yl)-4-oxo-butanoate (0.522 g, 1.7097 mmol, 40.0484% yield) obtained as a yellow solid. LCMS: (ESI, m/z): [M+H]⁺=306.126. ¹H NMR (400 MHz, DMSO-d₆) δ 10.34 (s, 1H), 7.67 (d, J=6.2 Hz, 1H), 7.28 (s, 0.5H), 7.24 (s, 0.5H), 4.90 (s, 1H), 4.80 (s, 1H), 4.66 (s, 1H), 4.57 (s, 1H), 4.05 (q, J=7.1 Hz, 2H), 3.96-3.90 (m, 3H), 2.66-2.59 (m, 2H), 2.59-2.52 (m, 2H), 1.18 (t, J=7.1 Hz, 3H).

### Step c: ethyl 4-[5-(hydroxymethyl)-6-methoxy-isoindolin-2-yl]-4-oxo-butanoate

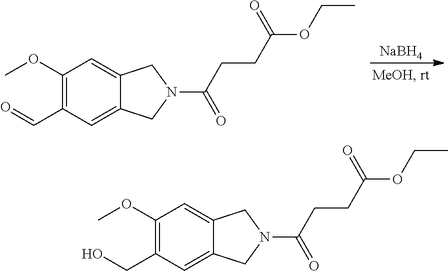 [SMILES: CCOC(=O)CCC(=O)N1Cc2cc(C=O)c(OC)cc2C1 ; "NaBH4" ; CCOC(=O)CCC(=O)N1Cc2cc(CO)c(OC)cc2C1]

NaBH4 (0.044 g, 1.1630 mmol) was added to ethyl 4-(5-formyl-6-methoxy-isoindolin-2-yl)-4-oxo-butanoate (0.515 g, 1.6867 mmol) in methanol (10 mL) at 20° C. The reaction mixture was stirred overnight at 20° C. The reaction mixture was concentrated and diluted with EA (10 mL), washed with water (10 mL) and brine (5 mL). The organics dried over Na₂SO₄, filtered and evaporated under reduced pressure. The pure fraction was concentrated and dried under vacuo. There was ethyl 4-[5-(hydroxymethyl)-6-methoxy-isoindolin-2-yl]-4-oxo-butanoate (0.327 g, 1.0640 mmol, 63.0787% yield) obtained as a yellow solid. LCMS: (ESI, m/z): [M+H]⁺=308.142. ¹H NMR (400 MHz, DMSO-d₆) δ 7.32 (d, J=6.4 Hz, 1H), 6.95 (s, 0.5H), 6.92 (s, 0.5H), 5.07-5.00 (m, 1H), 4.81 (s, 1H), 4.77 (s, 1H), 4.59 (s, 1H), 4.55 (s, 1H), 4.48 (d, J=5.6 Hz, 2H), 4.05 (q, J=7.1 Hz, 2H), 3.77 (s, 3H), 2.67-2.60 (m, 2H), 2.60-2.53 (m, 2H), 1.18 (t, J=7.1 Hz, 3H).

### Step d: diethyl 4,4′-(((methylazanediyl)bis(methylene))bis(6-methoxyisoindoline-5,2-diyl))bis(4-oxobutanoate)

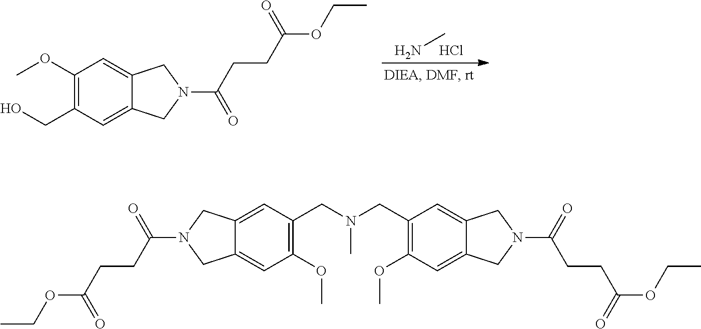

N,N-Diisopropylethylamine (0.113 ml, 683.7230 mol) was added to a solution of 4-[5-(hydroxymethyl)-6-methoxy-isoindolin-2-yl]-4-oxo-butanoate (0.067 g, 217.9990 mol) and Methylamine hydrochloride (0.018 g, 266.5964 mol) in N,N-Dimethylformamide (2 mL) at 20° C. The reaction mixture was stirred overnight at 20° C. The reaction mixture was evaporated under reduced pressure. The residue was purified on C18 column MeCN/Water (0-60%). The pure fraction was concentrated and dried under vacuo. There was diethyl 4,4′-(((methylazanediyl)bis(methylene))bis(6-methoxyisoindoline-5,2-diyl))bis(4-oxobutanoate) (0.071 g, 116.4492 μmol, 53.4173% yield) obtained as a off-white solid. LCMS: (ESI, m/z): [M+H]⁺=610.305. ¹H NMR (400 MHz, DMSO-d₆) δ 7.36 (s, 2H), 6.98 (s, 1H), 6.96 (s, 1H), 4.81 (s, 2H), 4.77 (s, 2H), 4.59 (s, 2H), 4.56 (s, 2H), 4.13-3.98 (m, 4H), 3.77 (s, 6H), 3.59 (s, 2H), 3.55 (s, 4H), 2.61 (d, J=5.0 Hz, 3H), 2.56 (d, J=4.7 Hz, 3H), 2.15 (s, 3H), 1.25-1.14 (m, 6H).

### Step e: 4,4′-(((methylazanediyl)bis(methylene))bis(6-methoxyisoindoline-5,2-diyl))bis(4-oxobutanoic acid)

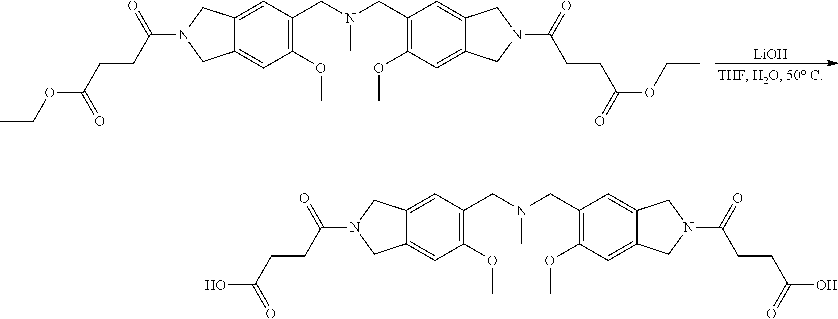

LiOH (0.010 g, 417.5662 mol) was added to a solution of diethyl 4,4′-(((methylazanediyl)bis(methylene))bis(6-methoxyisoindoline-5,2-diyl))bis(4-oxobutanoate) (0.069 g, 113.1689 μmol) in Tetrahydrofuran (2 mL) and Water (2 mL) at 20° C. The reaction mixture was stirred overnight at 50° C. The reaction mixture was adjusted to pH=6 with HCl (1 M). The reaction mixture was evaporated under reduced pressure. The residue was purified on C18 column MeCN/Water (0-80%). The pure fraction was concentrated and dried by lyohpilization. There was 4,4′-(((methylazanediyl)bis(methylene))bis(6-methoxyisoindoline-5,2-diyl))bis(4-oxobutanoic acid) (0.027 g, 48.7715 mol, 43.0962% yield) obtained as a off-white solid. LCMS: (ESI, m/z): [M−H]⁻=552.242. ¹H NMR (400 MHz, DMSO-d₆) δ 7.35 (s, 2H), 6.98 (s, 1H), 6.96 (s, 1H), 4.80 (s, 2H), 4.77 (s, 2H), 4.59 (s, 2H), 4.56 (s, 2H), 3.77 (s, 6H), 3.50 (s, 4H), 2.60-2.54 (m, 4H), 2.49-2.45 (m, 4H), 2.12 (s, 3H).

### Example I-8

### 4-(5-(3-((2-(3-carboxypropanoyl)-4-chloro-6-methoxyisoindolin-5-yl)oxy)propoxy)-6-methoxyisoindolin-2-yl)-4-oxobutanoic acid

Step a: ethyl 4-(4-chloro-5-hydroxy-6-methoxy-isoindolin-2-yl)-4-oxo-butanoate

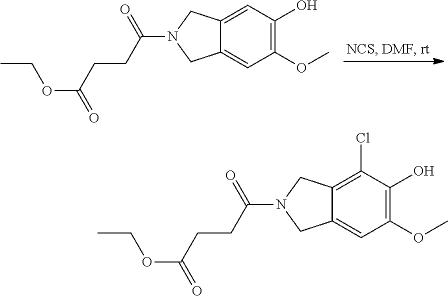

NCS (0.105 g, 786.3231 mol) was added to a solution of ethyl 4-(5-hydroxy-6-methoxyisoindolin-2-yl)-4-oxobutanoate (0.205 g, 698.9090 mol) in DMF (10 mL) at 0° C. under N₂ atmosphere. The reaction mixture was stirred for 12 h and warmed up to 20° C. naturally. The reaction mixture was purified on C18 column ACN/H₂O (0-25%). The pure fraction was concentrated and dried under vacuo. There was ethyl 4-(4-chloro-5-hydroxy-6-methoxy-isoindolin-2-yl)-4-oxo-butanoate (0.148 g, 451.5507 mol, 64.6079% yield) obtained as a white solid. LCMS: (ESI, m/z): [M+H]⁺=328.100. ¹H NMR (400 MHz, DMSO-d₆) δ 9.41 (s, 1H), 6.97 (s, 0.5H), 6.95 (s, 0.5H), 4.81 (s, 1H), 4.76 (s, 1H), 4.59 (s, 1H), 4.51 (s, 1H), 4.05 (q, J=7.2 Hz, 2H), 3.82 (s, 3H), 2.69-2.53 (m, 4H), 1.18 (t, J=7.2 Hz, 3H)

### Step b: ethyl 4-(5-(3-((4-chloro-2-(4-ethoxy-4-oxobutanoyl)-6-methoxyisoindolin-5-yl)oxy)propoxy)-6-methoxyisoindolin-2-yl)-4-oxobutanoate

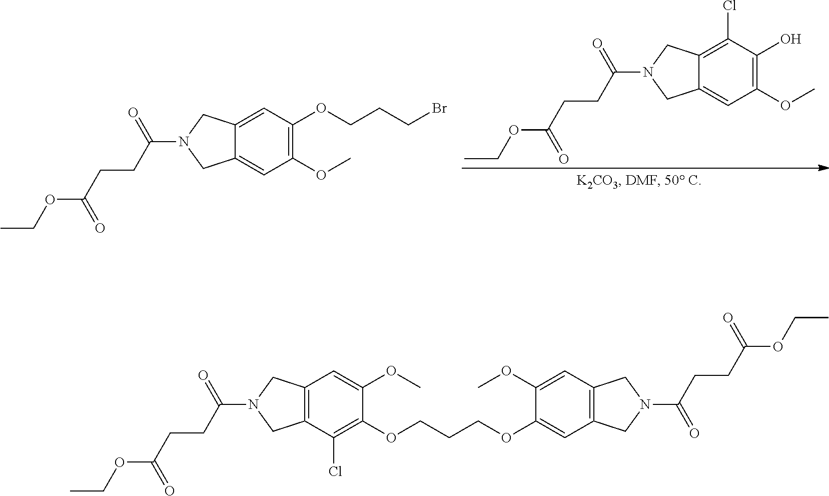 [SMILES: CCOC(=O)CCC(=O)N1Cc2cc(OC)c(OCCCBr)cc2C1 ; CCOC(=O)CCC(=O)N1Cc2cc(OC)c(O)c(Cl)c2C1 ; CCOC(=O)CCC(=O)N1Cc2cc(OC)c(OCCCOc3c(OC)cc4c(c3Cl)CN(C(=O)CCC(=O)OCC)C4)cc2C1]

Ethyl 4-(4-chloro-5-hydroxy-6-methoxyisoindolin-2-yl)-4-oxobutanoate (0.079 g, 241.0308 mol) and Potassium carbonate (0.098 g, 709.0890 mol) was added to a solution of ethyl 4-(5-(3-bromopropoxy)-6-methoxyisoindolin-2-yl)-4-oxobutanoate (0.082 g, 197.9290 mol) in DMF (2 mL) at 20° C. under N₂ atmosphere. The reaction mixture was heated to 50° C. and stirred overnight. The mixture was purified on C18 column ACN/H₂O (0.1% FA)(0-60%). The pure fraction was concentrated and dried under vacuo. There was ethyl 4-(5-(3-((4-chloro-2-(4-ethoxy-4-oxobutanoyl)-6-methoxyisoindolin-5-yl)oxy)propoxy)-6-methoxyisoindolin-2-yl)-4-oxobutanoate (0.096 g, 145.2043 mol, 73.3618% yield) obtained as a white solid. LCMS: (ESI, m/z): [M+H]⁺=661.240. ¹H NMR (400 MHz, DMSO-d₆) δ 7.08 (s, 0.5H), 7.05 (s, 0.5H), 7.01-6.92 (m, 2H), 4.85 (s, 1H), 4.76 (d, J=7.3 Hz, 3H), 4.62 (s, 1H), 4.52 (d, J=6.5 Hz, 3H), 4.16 (t, J=7.0 Hz, 2H), 4.11 (t, J=6.1 Hz, 2H), 4.05 (q, J=7.0 Hz, 4H), 3.78 (s, 3H), 3.74 (s, 3H), 2.69-2.58 (m, 4H), 2.58-2.53 (m, 4H), 2.18-2.09 (m, 2H), 1.18 (t, J=7.1 Hz, 6H).

### Step c: 4-(5-(3-((2-(3-carboxypropanoyl)-4-chloro-6-methoxyisoindolin-5-yl)oxy)propoxy)-6-methoxyisoindolin-2-yl)-4-oxobutanoic acid

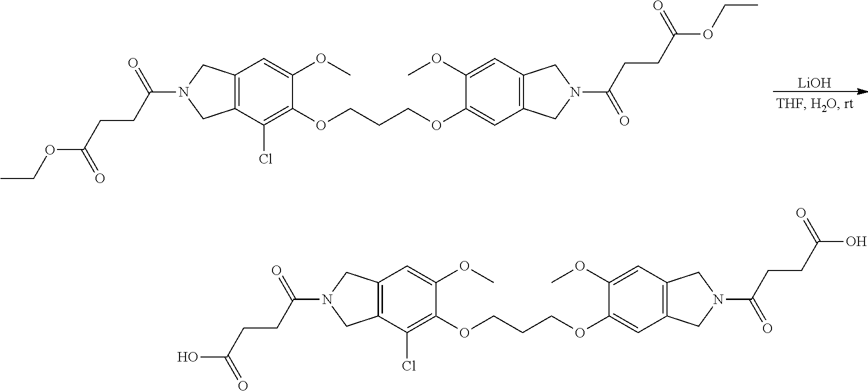 [SMILES: CCOC(=O)CCC(=O)N1Cc2cc(OC)c(OCCCOc3c(OC)cc4c(c3Cl)CN(C(=O)CCC(=O)OCC)C4)cc2C1 ; "LiOH" ; COc1cc2c(cc1OCCCOc1c(OC)cc3c(c1Cl)CN(C(=O)CCC(=O)O)C3)CN(C(=O)CCC(=O)O)C2]

LiOH (0.054 g, 2.2549 mmol) was added to a mixture of ethyl 4-(5-(3-((4-chloro-2-(4-ethoxy-4-oxobutanoyl)-6-methoxyisoindolin-5-yl)oxy)propoxy)-6-methoxyisoindolin-2-yl)-4-oxobutanoate (0.060 g, 90.7527 mol) in THF (2 mL), EtOH (1 mL) and Water (2 mL) at 20° C. . The reaction mixture was stirred for 2 h at 20° C. The reaction mixture was adjusted to pH=3 with HCl (1 mol/L). The mixture was purified on C18 column ACN/H₂O (0.1% FA)(0-40%). The pure fraction was concentrated and dried by lyophilization. There was 4-(5-(3-((2-(3-carboxypropanoyl)-4-chloro-6-methoxyisoindolin-5-yl)oxy)propoxy)-6-methoxyisoindolin-2-yl)-4-oxobutanoic acid (0.044 g, 72.7235 mol, 80.1337% yield) obtained as a white solid. LCMS: (ESI, m/z): [M+H]⁺15=605.180. ¹H NMR (400 MHz, DMSO-d₆) δ 7.08 (s, 0.5H), 7.05 (s, 0.5H), 7.00-6.93 (m, 2H), 4.85 (s, 1H), 4.75 (d, J=6.7 Hz, 3H), 4.62 (s, 1H), 4.53 (d, J=4.9 Hz, 3H), 4.17 (t, J=6.4 Hz, 2H), 4.12 (t, J=6.1 Hz, 2H), 3.78 (s, 3H), 3.74 (s, 3H), 2.61-2.54 (m, 4H), 2.53-2.47 (m, 4H), 2.17-2.09 (m, 2H).

### Example I-9

### 4,4′-((propane-1,3-diylbis(oxy))bis(4-fluoro-6-methoxyisoindoline-5,2-diyl))bis(4-oxobutanoic acid)

### Step a: ethyl 4-(5-(3-bromopropoxy)-4-fluoro-6-methoxyisoindolin-2-yl)-4-oxobutanoate

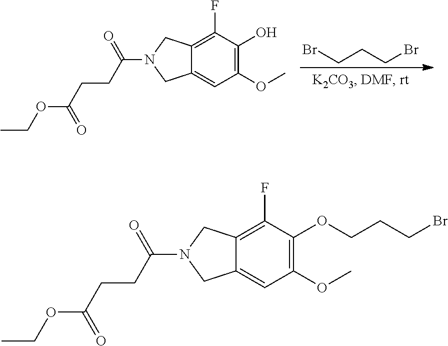

1,3-dibromopropane (0.708 g, 3.5069 mmol) was added to a mixture of ethyl 4-(4-fluoro-5-hydroxy-6-methoxyisoindolin-2-yl)-4-oxobutanoate (0.217 g, 697.0656 mol) and Potassium carbonate (0.346 g, 2.5035 mmol) in DMF (5 mL) at 20° C. The reaction mixture was stirred for 8 h at 20° C. The reaction mixture was purified on C18 column ACN/H₂O (0.1% FA)(0-45%). The pure fraction was concentrated and dried under vacuo.

There was ethyl 4-(5-(3-bromopropoxy)-4-fluoro-6-methoxyisoindolin-2-yl)-4-oxobutanoate (0.211 g, 488.1091 μmol, 70.0234% yield) obtained as a white solid. LCMS: (ESI, m/z): [M+H]⁺=432.070.

### Step b: diethyl 4,4′-((propane-1,3-diylbis(oxy))bis(4-fluoro-6-methoxyisoindoline-5,2-diyl))bis(4-oxobutanoate)

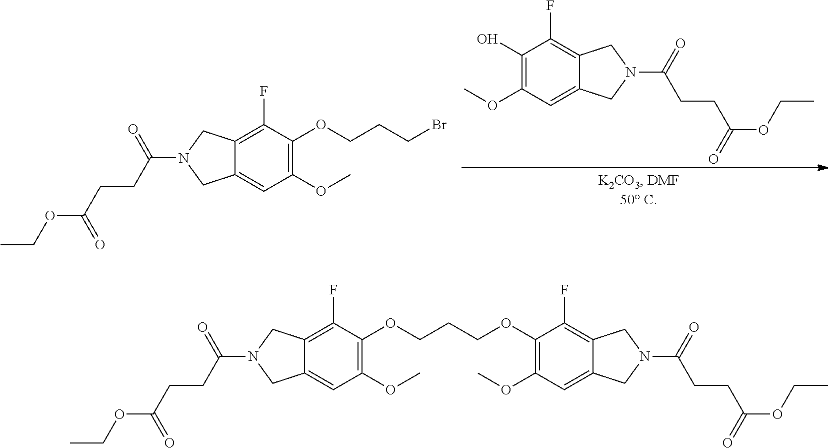

Potassium carbonate (0.111 g, 803.1518 mol) was added to a solution of ethyl 4-(5-(3-bromopropoxy)-4-fluoro-6-methoxyisoindolin-2-yl)-4-oxobutanoate (0.072 g, 166.5586 mol) and ethyl 4-(4-fluoro-5-hydroxy-6-methoxyisoindolin-2-yl)-4-oxobutanoate (0.051 g, 163.8266 mol) in DMF (3 mL) at 20° C. under N₂ atmosphere. The reaction mixture was stirred for 8 h at 50° C. The reaction mixture was purified on C18 column ACN/H₂O (0.1% FA)(0-50%). The pure fraction was concentrated and dried by lyophilization. There was diethyl 4,4′-((propane-1,3-diylbis(oxy))bis(4-fluoro-6-methoxyisoindoline-5,2-diyl))bis(4-oxobutanoate) (0.085 g, 128.2683 μmol, 78.2952% yield) obtained as a white solid. LCMS: (ESI, m/z): [M+H]⁺=663.270. ¹H NMR (400 MHz, DMSO-d₆) δ 6.92 (s, 1H), 6.90 (s, 1H), 4.83 (s, 2H), 4.81 (s, 2H), 4.57 (s, 4H), 4.16 (t, J=6.0 Hz, 4H), 4.05 (q, J =7.2 Hz, 4H), 3.83-3.73 (m, 6H), 2.67-2.53 (m, 8H), 2.06-1.94 (m, 2H), 1.18 (t, J=7.2 Hz, 6H).

### Step c: 4,4′-((propane-1,3-diylbis(oxy))bis(4-fluoro-6-methoxyisoindoline-5,2-diyl))bis(4-oxobutanoic acid)

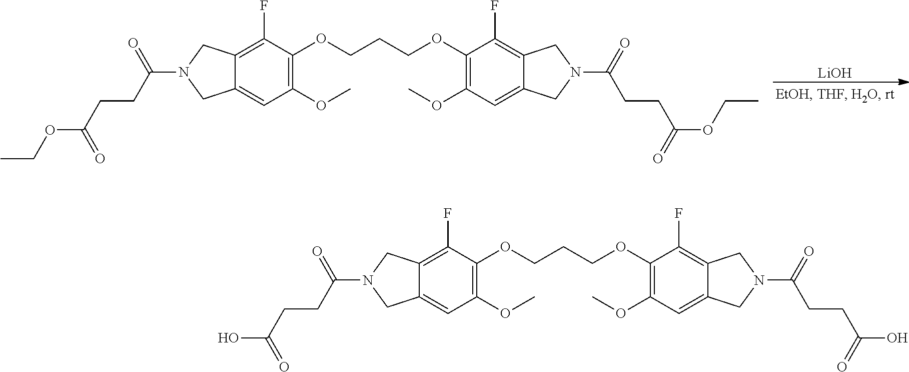 [SMILES: CCOC(=O)CCC(=O)N1Cc2cc(OC)c(OCCCOc3c(OC)cc4c(c3F)CN(C(=O)CCC(=O)OCC)C4)c(F)c2C1 ; "LiOH" ; COc1cc2c(c(F)c1OCCCOc1c(OC)cc3c(c1F)CN(C(=O)CCC(=O)O)C3)CN(C(=O)CCC(=O)O)C2]

LiOH (0.044 g, 1.8373 mmol) was added to a solution of diethyl 4,4′-((propane-1,3-diylbis(oxy))bis(4-fluoro-6-methoxyisoindoline-5,2-diyl))bis(4-oxobutanoate) (0.057 g, 86.0153 mol) in THF (2 mL), H₂O (2 mL) and EtOH (1 mL) at 20° C. . The reaction mixture was stirred for 1 h at 20° C. The reaction mixture was adjusted to pH=3 with HCl (1 mol/L), The reaction mixture was evaporated under reduced pressure. The residue was purified on C18 column ACN/H₂O (0.1% FA)(0-40%). The pure fraction was concentrated and dried under vacuo. There was 4,4′-((propane-1,3-diylbis(oxy))bis(4-fluoro-6-methoxyisoindoline-5,2-diyl))bis(4-oxobutanoic acid) (0.042 g, 69.2421 mol, 80.4998% yield) obtained as a white solid. LCMS: (ESI, m/z): [M+H]⁺=607.200. ¹H NMR (400 MHz, DMSO-d₆) δ 6.92 (s, 1H), 6.90 (s, 1H), 4.82 (s, 2H), 4.80 (s, 2H), 4.58-4.56 (m, 4H), 4.16 (t, J=6.2 Hz, 4H), 3.78 (s, 6H), 2.64-2.54 (m, 4H), 2.49-2.44 (m, 4H), 2.07-1.94 (m, 2H).

### Example I-10

### 4,4′-(((2-methylenepropane-1,3-diyl)bis(oxy))bis(6-methoxyisoindoline-5,2-diyl))bis(4-oxobutanoic acid)

### Step a: diethyl 4,4′-(((2-methylenepropane-1,3-diyl)bis(oxy))bis(6-methoxyisoindoline-5,2-diyl))bis(4-oxobutanoate)

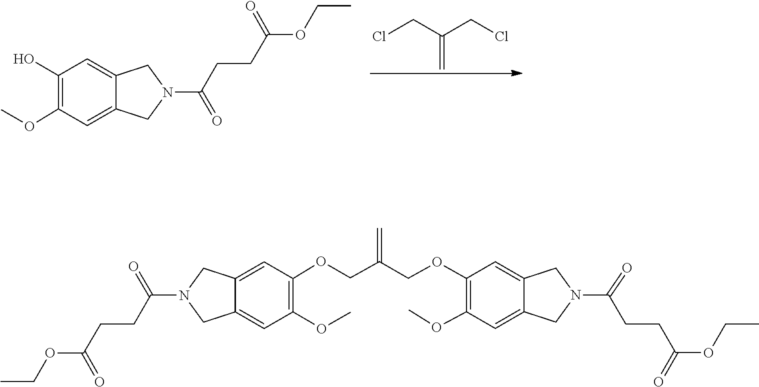

Potassium carbonate (0.151 g, 1.0926 mmol) was added to a solution of ethyl 4-(5-hydroxy-6-methoxyisoindolin-2-yl)-4-oxobutanoate (0.099 g, 337.5219 mol) and 3-Chloro-2-(chloromethyl)prop-1-ene (0.034 g, 151.7938 mol) in N,N-Dimethylformamide (5 mL) at 20° C. The reaction mixture was stirred overnight at 60° C. The reaction mixture was purified on C-18 column MeCN/water (0-100%). The pure fraction was concentrated and dried under vacuo. There was diethyl 4,4′-(((2-methylenepropane-1,3-diyl)bis(oxy))bis(6-methoxyisoindoline-5,2-diyl))bis(4-oxobutanoate) (0.10 g, 156.5673 gmol, 103.1447% yield) obtained as a white solid. LCMS: (ESI, m/z): [M+H]⁺=639.284. ¹H NMR (400 MHz, DMSO-d₆) δ 7.13-6.88 (m, 4H), 5.33 (s, 2H), 4.83-4.58 (m, 8H), 4.57-4.40 (m, 4H), 4.12-3.96 (m, 4H), 3.82-3.69 (m, 6H), 2.66-2.53 (m, 8H), 1.25-1.11 (m, 6H).

### Step b: 4,4′-(((2-methylenepropane-1,3-diyl)bis(oxy))bis(6-methoxyisoindoline-5,2-diyl))bis(4-oxobutanoic acid)

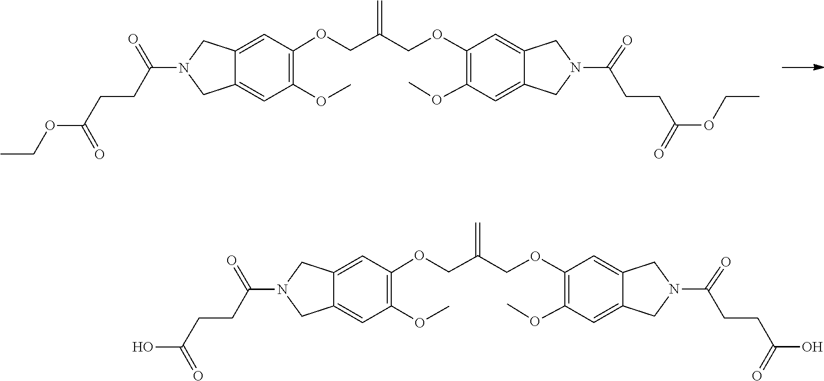 [SMILES: C=C(COc1cc2c(cc1OC)CN(C(=O)CCC(=O)OCC)C2)COc1cc2c(cc1OC)CN(C(=O)CCC(=O)OCC)C2 ; C=C(COc1cc2c(cc1OC)CN(C(=O)CCC(=O)O)C2)COc1cc2c(cc1OC)CN(C(=O)CCC(=O)O)C2]

LiOH (0.014 g, 584.5926 mol) was added to a solution of diethyl 4,4′-(((2-methylenepropane-1,3-diyl)bis(oxy))bis(6-methoxyisoindoline-5,2-diyl))bis(4-oxobutanoate) (0.08 g, 125.2538 mol) in Tetrahydrofuran (10 mL) and Water (2.5 mL) at 20° C. The reaction mixture was stirred for 2 h at 20° C. The reaction mixture was evaporated under reduced pressure. The residue was purified on C-18 column MeCN/water (0-100%). The pure fraction was concentrated and dried under vacuo. There was 4,4′-(((2-methylenepropane-1,3-diyl)bis(oxy))bis(6-methoxyisoindoline-5,2-diyl))bis(4-oxobutanoic acid) (0.028 g, 48.0607 mol, 38.3706% yield) obtained as a white solid. LCMS: (ESI, m/z): [M+H]⁺=583.221. ¹H NMR (400 MHz, DMSO-d₆) δ 7.01-6.89 (m, 4H), 5.32 (s, 2H), 4.78-4.59 (m, 8H), 4.55-4.43 (m, 4H), 3.75 (s, 6H), 2.56 (d, J=5.5 Hz, 4H), 2.47-2.41 (m, 4H).

### Example I-11

### 4,4′-(((2-methylenepropane-1,3-diyl)bis(oxy))bis(4-chloro-6-methoxyisoindoline-5,2-diyl))bis(4-oxobutanoic acid)

### Step a: ethyl 4-[4-chloro-5-[2-(chloromethyl)allyloxy]-6-methoxy-isoindolin-2-yl]-4-oxo-butanoate

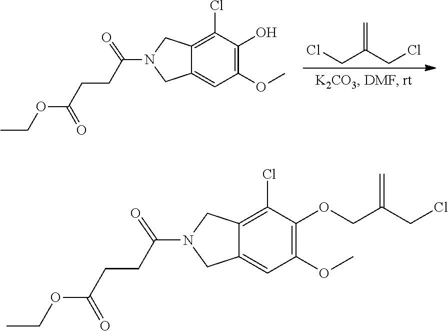

3-Chloro-2-(chloromethyl)prop-1-ene (0.507 g, 2.2635 mmol) was added to a mixture of Potassium carbonate (0.202 g, 1.4616 mmol) and ethyl 4-(4-chloro-5-hydroxy-6-methoxyisoindolin-2-yl)-4-oxobutanoate (0.154 g, 469.8574 mol) in DMF (5 mL) at 20° C. under N₂ atmosphere. The reaction mixture was stirred for 4 h at 20° C. The mixture was purified on C18 column ACN/H₂O (0.1% FA)(0-50%). The pure fraction was concentrated and dried under vacuo. There was ethyl 4-[4-chloro-5-[2-(chloromethyl)allyloxy]-6-methoxy-isoindolin-2-yl]-4-oxo-butanoate (0.151 g, 362.7238 mol, 77.1987% yield) obtained as a white solid. LCMS: (ESI, m/z): [M+H]⁺=328.090.

### Step b: diethyl 4,4′-(((2-methylenepropane-1,3-diyl)bis(oxy))bis(4-chloro-6-methoxyisoindoline-5,2-diyl))bis(4-oxobutanoate)

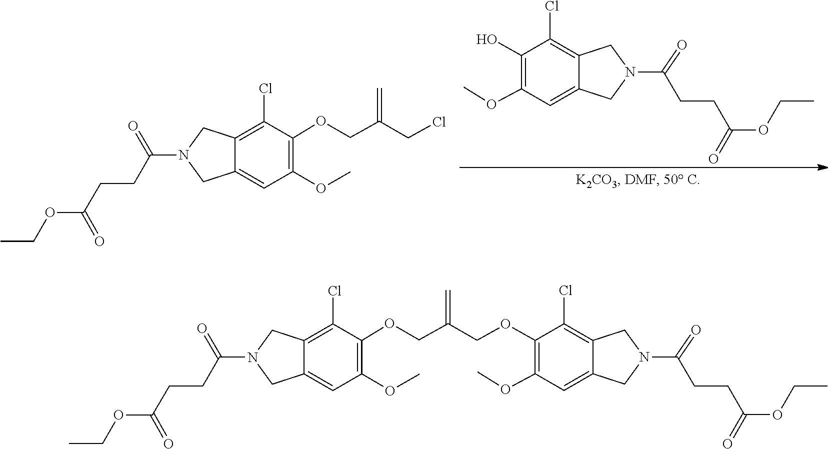

Potassium carbonate (0.105 g, 759.7382 mol) was added to a solution of ethyl 4-(4-chloro-5-hydroxy-6-methoxyisoindolin-2-yl)-4-oxobutanoate (0.068 g, 207.4693 mol) and ethyl 4-(4-chloro-5-((2-(chloromethyl)allyl)oxy)-6-methoxyisoindolin-2-yl)-4-oxobutanoate (0.084 g, 187.1929 mol) in DMF (3 mL) at 20° C. under N₂ atmosphere. The reaction mixture was stirred for 3 h at 50° C. The reaction mixture was purified on C18 column ACN/H₂O (0.1% FA)(0-60%). The pure fraction was concentrated and dried under vacuo. There was diethyl 4,4′-(((2-methylenepropane-1,3-diyl)bis(oxy))bis(4-chloro-6-methoxyisoindoline-5,2-diyl))bis(4-oxobutanoate) (0.083 g, 119.3245 mol, 63.7441% yield) obtained as a white solid. LCMS: (ESI, m/z): [M+H]⁺=707.210. ¹H NMR (400 MHz, DMSO-d₆) δ 7.10 (s, 1H), 7.07 (s, 1H), 5.36 (s, 2H), 4.86 (s, 2H), 4.77 (s, 2H), 4.67 (s, 4H), 4.64 (s, 2H), 4.51 (d, J=4.7 Hz, 2H), 4.06 (q, J=7.1 Hz, 4H), 3.82 (s, 6H), 2.71-2.59 (m, 4H), 2.59-2.53 (m, 4H), 1.19 (t, J=7.1 Hz, 6H).

### Step c: 4,4′-(((2-methylenepropane-1,3-diyl)bis(oxy))bis(4-chloro-6-methoxyisoindoline-5,2-diyl))bis(4-oxobutanoic acid)

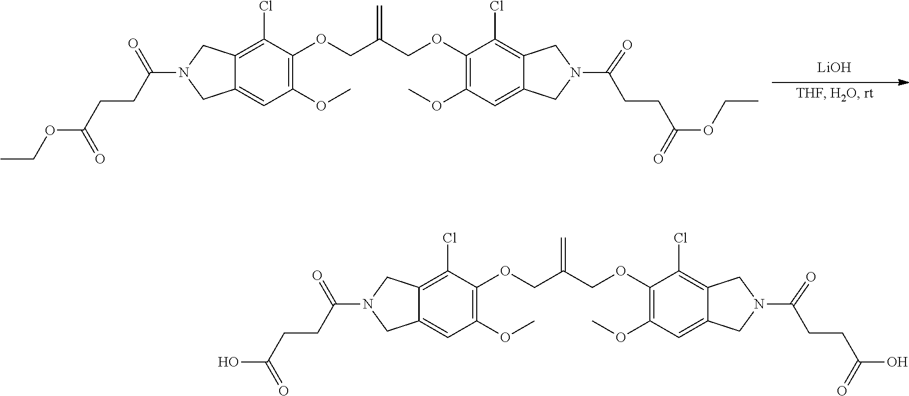 [SMILES: C=C(COc1c(OC)cc2c(c1Cl)CN(C(=O)CCC(=O)OCC)C2)COc1c(OC)cc2c(c1Cl)CN(C(=O)CCC(=O)OCC)C2 ; "LiOH" ; "THF, H2O, rt" ; C=C(COc1c(OC)cc2c(c1Cl)CN(C(=O)CCC(=O)O)C2)COc1c(OC)cc2c(c1Cl)CN(C(=O)CCC(=O)O)C2]

LiOH (0.038 g, 1.5868 mmol) was added to a solution of diethyl 4,4′-(((2-methylenepropane-1,3-diyl)bis(oxy))bis(4-chloro-6-methoxyisoindoline-5,2-diyl))bis(4-oxobutanoate) (0.046 g, 65.0091 mol) in THF (1.5 mL) and Water (1.5 mL) at 20° C. . The reaction mixture was stirred for 1 h at 20° C. The reaction mixture was adjusted to pH=3 with HCl (1 mol/L), The reaction mixture was evaporated under reduced pressure. The residue was purified on C18 column ACN/H₂O (0.1% FA)(0-40%). The pure fraction was concentrated and dried by lyophilization. There was 4,4′-(((2-methylenepropane-1,3-diyl)bis(oxy))bis(4-chloro-6-methoxyisoindoline-5,2-diyl))bis(4-oxobutanoic acid) (0.037 g, 56.7931 mol, 87.3618% yield) obtained as a white solid. LCMS: (ESI, m/z): [M−H]⁻=649.140. ¹H NMR (400 MHz, DMSO-d₆) δ 7.10 (s, 1H), 7.07 (d, J=3.1 Hz, 1H), 5.36 (s, 2H), 4.86 (s, 2H), 4.77 (s, 2H), 4.67 (s, 4H), 4.63 (s, 2H), 4.51 (d, J=6.0 Hz, 2H), 3.82 (s, 6H), 2.65-2.54 (m, 6H), 2.50-2.46 (m, 2H).

### Example I-12

### 4,4′-((propane-1,3-diylbis(oxy))bis(4-chloro-6-methoxyisoindoline-5,2-diyl))bis(4-oxobutanoic acid)

### Step a: ethyl 4-[5-(3-bromopropoxy)-4-chloro-6-methoxy-isoindolin-2-yl]-4-oxo-butanoate

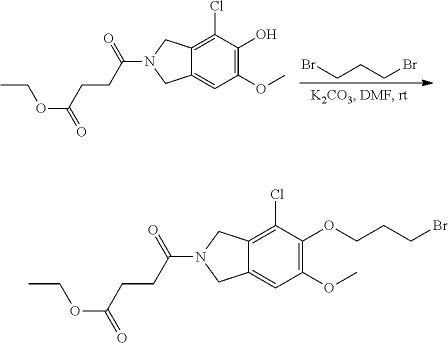

1,3-Dibromopropane (0.495 g, 2.4519 mmol) was added to a mixture of K₂CO₃ (0.199 g, 1.4399 mmol), ethyl 4-(4-chloro-5-hydroxy-6-methoxyisoindolin-2-yl)-4-oxobutanoate (0.149 g, 454.6023 mol) in DMF (5 mL) at 20° C. under N₂ atmosphere. The reaction mixture was stirred for 4 h at 20° C. The reaction mixture was purified on C18 column ACN/H₂O (0.1% FA)(0-50%). The pure fraction was concentrated and dried under vacuo. There was ethyl 4-[5-(3-bromopropoxy)-4-chloro-6-methoxy-isoindolin-2-yl]-4-oxo-butanoate (0.163 g, 363.2433 mol, 79.9035% yield) obtained as a white solid. LCMS: (ESI, m/z): [M+H]⁺=448.040. ¹H NMR (400 MHz, DMSO-d₆) δ 7.10 (s, 0.5H), 7.08 (s, 0.5H), 4.87 (s, 1H), 4.79 (s, 1H), 4.64 (s, 1H), 4.53 (s, 1H), 4.06-4.02 (m, 4H), 3.83 (d, J=2.4 Hz, 3H), 3.78-3.70 (m, 2H), 2.71-2.53 (m, 4H), 2.23-2.20 (m, 2H), 1.18 (t, J=7.2 Hz, 3H).

### Step b: diethyl 4,4′-((propane-1,3-diylbis(oxy))bis(4-chloro-6-methoxyisoindoline-5,2-diyl))bis(4-oxobutanoate)

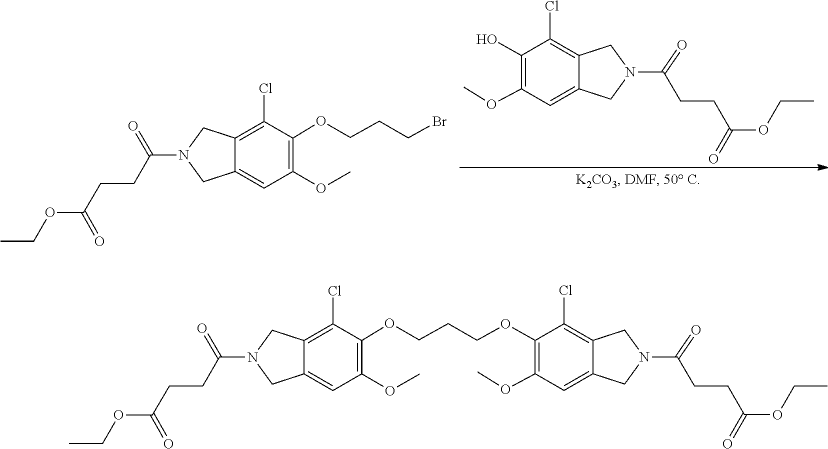

Potassium carbonate (0.105 g, 759.7382 mol) was added to a solution of ethyl 4-(4-chloro-5-hydroxy-6-methoxyisoindolin-2-yl)-4-oxobutanoate (0.068 g, 207.4693 mol) and ethyl 4-[5-(3-bromopropoxy)-4-chloro-6-methoxy-isoindolin-2-yl]-4-oxo-butanoate (0.084 g, 187.1929 mol) in DMF (3 mL) at 20° C. under N₂ atmosphere. The reaction mixture was stirred for 4 h at 50° C. The mixture was purified on C18 column ACN/H₂O (0.1% FA)(0-60%). The pure fraction was concentrated and dried under vacuo. There was diethyl 4,4′-((propane-1,3-diylbis(oxy))bis(4-chloro-6-methoxyisoindoline-5,2-diyl))bis(4-oxobutanoate) (0.083 g, 119.3245 mol, 63.7441% yield) obtained as a white solid. LCMS: (ESI, m/z): [M+H]=695.210. ¹H NMR (400 MHz, DMSO-d₆) δ 7.08 (s, 1H), 7.06 (s, 1H), 4.86 (s, 2H), 4.77 (s, 2H), 4.63 (s, 2H), 4.52 (s, 2H), 4.17 (t, J=6.4 Hz, 4H), 4.05 (q, J=7.1 Hz, 4H), 3.82-3.76 (m, 6H), 2.68-2.59 (m, 4H), 2.58-2.53 (m, 4H), 2.11 (d, J=7.0 Hz, 2H), 1.18 (t, J=7.1 Hz, 6H).

### Step c: 4,4′-((propane-1,3-diylbis(oxy))bis(4-chloro-6-methoxyisoindoline-5,2-diyl))bis(4-oxobutanoic acid)

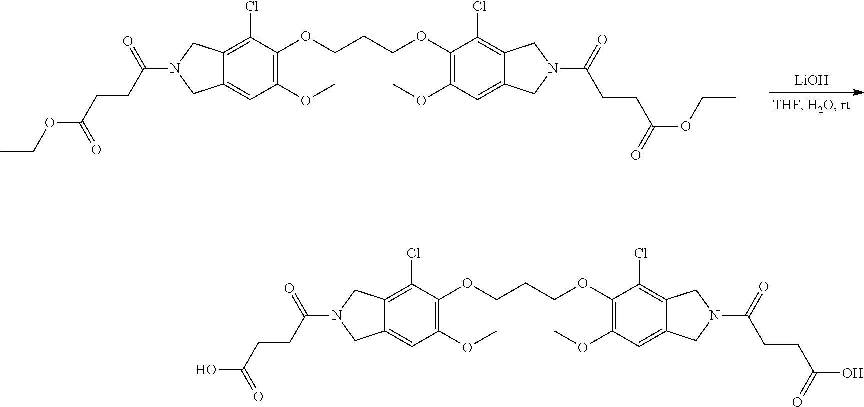 [SMILES: CCOC(=O)CCC(=O)N1Cc2cc(OC)c(OCCCOc3c(OC)cc4c(c3Cl)CN(C(=O)CCC(=O)OCC)C4)c(Cl)c2C1 ; "LiOH" ; COc1cc2c(c(Cl)c1OCCCOc1c(OC)cc3c(c1Cl)CN(C(=O)CCC(=O)O)C3)CN(C(=O)CCC(=O)O)C2]

LiOH (0.051 g, 2.1296 mmol) was added to a solution of diethyl 4,4′-((propane-1,3-diylbis(oxy))bis(4-chloro-6-methoxyisoindoline-5,2-diyl))bis(4-oxobutanoate) (0.053 g, 76.1951 mol) in THF (2 mL) and Water (2 mL) at 20° C. . The reaction mixture was stirred for 1 h at 20° C. The reaction mixture was adjusted to pH=3 with HCl (1 mol/L), The mixture was evaporated under reduced pressure. The residue was purified on C18 column ACN/H₂O (0.1% FA)(0-40%). The pure fraction was concentrated and dried by lyophilization. There was 4,4′-((propane-1,3-diylbis(oxy))bis(4-chloro-6-methoxyisoindoline-5,2-diyl))bis(4-oxobutanoic acid) (0.044 g, 68.8063 mol, 90.3028% yield) obtained as a white solid. LCMS: (ESI, m/z): [M−H]⁻=637.140. ¹H NMR (400 MHz, DMSO-d₆) δ 7.08 (s, 1H), 7.06 (s, 1H), 4.85 (s, 2H), 4.77 (s, 2H), 4.63 (s, 2H), 4.51 (d, J=4.1 Hz, 2H), 4.17 (t, J=6.3 Hz, 4H), 3.80 (d, J=2.6 Hz, 6H), 2.63-2.53 (m, 5H), 2.50-2.46 (m, 3H), 2.16-2.05 (m, 2H).

### Example I-13

### 4-(5-(3-((2-(3-carboxypropanoyl)-4-chloro-6-methoxyisoindolin-5-yl)oxy)propoxy)-4-fluoro-6-methoxyisoindolin-2-yl)-4-oxobutanoic acid

### Step a: 4-fluoro-5-methoxy-isoindoline

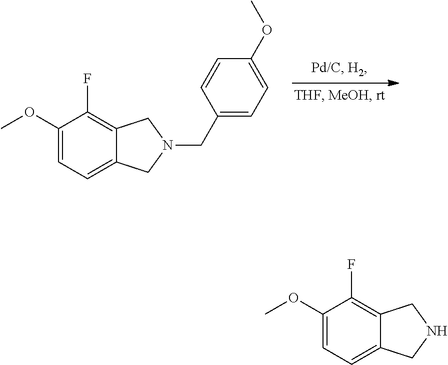 [SMILES: COc1ccc(CN2Cc3ccc(OC)c(F)c3C2)cc1 ; COc1ccc2c(c1F)CNC2]

10% Pd/C (7.68 g, contain 55% H₂O) was added to a solution of 4-fluoro-5-methoxy-2-[(4-methoxyphenyl)methyl]isoindoline (7.18 g, 24.9889 mmol) in THF (70 mL) and MeOH (70 mL) at 20° C. The reaction mixture was stirred overnight at 20° C. The reaction mixture was filtered and the filter cake was washed with MeOH (70 mL). The filtrate was evaporated under reduced pressure. There was 4-fluoro-5-methoxy-isoindoline (4.88 g, 29.1901 mmol, 100% yield) obtained as a brown oil, which was used directly in the next step.

LCMS: (ESI, m/z): [M+H]⁺=168.200.

### Step b: 4-fluoroisoindolin-5-ol hydrobromate

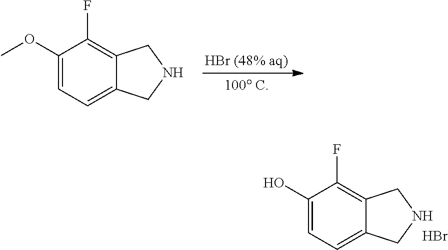

4-fluoro-5-methoxy-isoindoline (4.5 g, 26.9171 mmol) was dissolved in Hydrobromic acid (120 mL, 48% aq) at 20° C. The reaction mixture was heated to 100° C. and stirred for 6 h. The resulting reaction mixture was evaporated under reduced pressure. The residue was treated with n-hexane/EA (1:2). The solid was collected by filtration, and dried under reduced pressure. There was 4-fluoroisoindolin-5-ol hydrobromate (4.70 g, 20.0799 mmol, 74.60% yield) obtained as a brown solid. LCMS: (ESI, m/z): [M+H]⁺=154.200. ¹H NMR (400 MHz, DMSO-d₆) δ 10.09 (s, 1H), 9.58 (s, 2H), 7.10-6.93 (m, 2H), 4.57 (s, 2H), 4.44 (s, 2H).

### Step c: tert-butyl 4-fluoro-5-hydroxy-isoindoline-2-carboxylate

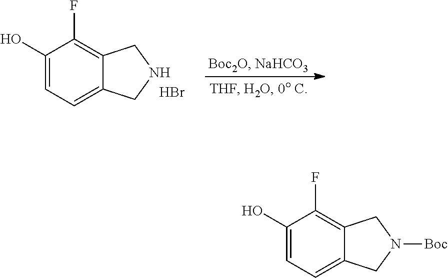 [SMILES: Oc1ccc2c(c1F)CNC2 ; Br ; CC(C)(C)OC(=O)N1Cc2ccc(O)c(F)c2C1]

NaHCO₃ (3.43 g, 40.8301 mmol) was added to a solution of 4-fluoroisoindolin-5-olhydrobromate (4.70 g, 20.0799 mmol) in Water (80 mL) at 0° C. After the reaction mixture was stirred for 20 min, THF (60 mL) was added to the reaction mixture, and Di-tert-butyl dicarbonate (4.45 g, 20.3898 mmol) in THF (20 mL) was added dropwise to the mixture at 0° C. After stirring for 20 min, the reaction mixture was warmed to 20° C. and stirred for 2 h. The resulting reaction mixture was diluted with EA (150 mL) and washed with brine (150 mL). The organic layer was dried over Na₂SO₄, filtered and evaporated under reduced pressure. The residue was purified by flash chromatography, eluting with ethyl acetate in n-hexane (0-40%). The pure fraction was concentrated and dried under reduced pressure. There was tert-butyl 4-fluoro-5-hydroxy-isoindoline-2-carboxylate (3.14 g, 12.3979 mmol, 60.78% yield) obtained as a reddish brown solid. LCMS: (ESI, m/z): [M−H]⁻=252.000. ¹H NMR (400 MHz, CDCl₃-d) 6 7.00-6.93 (m, 1H), 6.93-6.83 (m, 1H), 4.72 (s, 2H), 4.64 (s, 2H), 1.54 (s, 9H).

### Step d: tert-butyl 6-bromo-4-fluoro-5-hydroxy-isoindoline-2-carboxylate

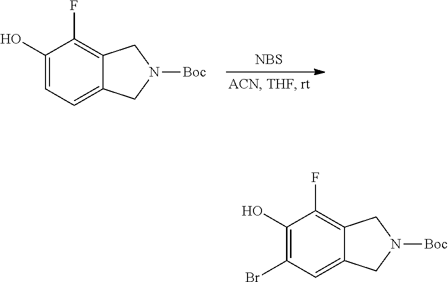 [SMILES: CC(C)(C)OC(=O)N1Cc2ccc(O)c(F)c2C1 ; "NBS" ; CC(C)(C)OC(=O)N1Cc2cc(Br)c(O)c(F)c2C1]

NBS (2.20 g, 12.3607 mmol) was added to a solution of tert-butyl 4-fluoro-5-hydroxy-isoindoline-2-carboxylate (3.10 g, 12.2400 mmol) in ACN (40 mL) and THF (20 mL) at 0° C. The reaction mixture was stirred for 2.5 h at 20° C. The reaction mixture was concentrated under reduced pressure. The residue was dissolved with EA/MeOH (200 mL/10 mL), and washed with H₂O (200 mL). The organic layer was dried over Na₂SO₄, filtered and evaporated in vacuo. The residue was purified by flash chromatography, eluting with MeOH in DCM (0-5%). The pure fraction was concentrated and dried under reduced pressure. There was tert-butyl 6-bromo-4-fluoro-5-hydroxy-isoindoline-2-carboxylate (2.70 g, 8.1285 mmol, 66.34% yield) obtained as a white solid. LCMS: (ESI, m/z): [M−H]⁻=331.950. ¹H NMR (400 MHz, DMSO-d₆) δ 10.36 (s, 1H), 7.33 (s, 0.5H), 7.32 (s, 0.5H), 4.58 (s, 1H), 4.55 (s, 1H), 4.52 (s, 1H), 4.50 (s, 1H), 1.45 (s, 9H).

### Step e: tert-butyl 5-benzyloxy-6-bromo-4-fluoro-isoindoline-2-carboxylate

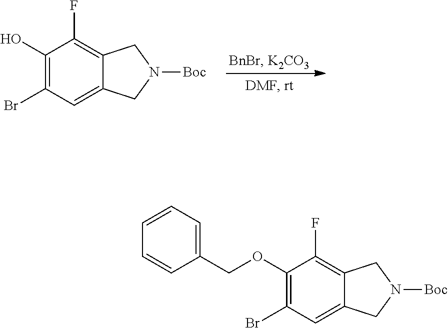

K₂CO₃ (2.20 g, 15.9183 mmol) was added to a solution of tert-butyl 6-bromo-4-fluoro-5-hydroxy-isoindoline-2-carboxylate (2.70 g, 8.1285 mmol) in DMF (50 mL) at 0° C. After the reaction mixture was stirred for 0.5 h at 0° C., BnBr (1.90 g, 11.1089 mmol) was added to the reaction mixture. The reaction mixture was stirred for 9 h at 20° C. The reaction mixture was diluted with EA (150 mL) and washed with water (150 mL) and brine (2×150 mL). The organics dried over Na₂SO₄, filtered and evaporated under reduced pressure. The residue was purified by flash chromatography, eluting with ethyl acetate in n-hexane (0-10%). The pure fraction was concentrated and dried under reduced pressure. There was tert-butyl 5-benzyloxy-6-bromo-4-fluoro-isoindoline-2-carboxylate (3.43 g, 8.1224 mmol, 99.93% yield) obtained as a white semi-solid. LCMS: (ESI, m/z): [M-tBu+ACN]⁺=407.050. ¹H NMR (400 MHz, CDCl₃-d) 6 7.54 (d, J=7.3 Hz, 2H), 7.45-7.35 (m, 3H), 7.27 (s, 0.5H), 7.20 (s, 0.5H), 5.13 (s, 2H), 4.70 (s, 1H), 4.65 (s, 2H), 4.62 (s, 1H), 1.54 (s, 9H).

### Step f: tert-butyl 5-benzyloxy-4-fluoro-6-hydroxy-isoindoline-2-carboxylate

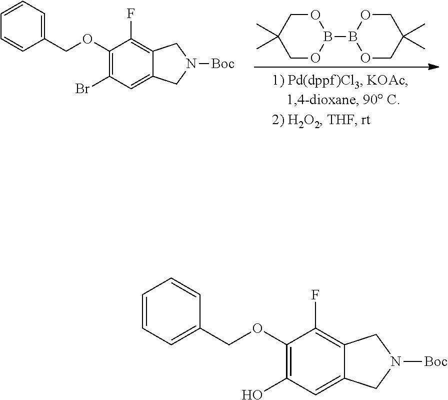

[1,1′-Bis(diphenylphosphino)ferrocene]dichloropalladium(II) (1.12 g, 1.5307 mmol) was added to a solution of tert-butyl 5-benzyloxy-6-bromo-4-fluoro-isoindoline-2-carboxylate (3.30 g, 7.8146 mmol), bis(neopentylglycolato)diboron (5.46 g, 24.1716 mmol) and KOAc (2.32 g, 23.6392 mmol) in 1,4-Dioxane (80 mL) at 20° C. The reaction mixture was heated to 90° C. and stirred overnight under nitrogen atmosphere. The reaction mixture was diluted with EA (100 mL) and filtered through a celite. The filtrate was evaporated under reduced pressure. H₂O₂(88.1974 mmol, 10 mL, 30% aqueous solution) was added to a mixture of the residue and NaHCO₃ (11.9038 mmol, 20 mL, 5% aqueous solution) in THF (80 mL) at 0° C. The reaction mixture was stirred for 2 h at 20° C. The resulting reaction mixture was diluted with brine (150 mL) and saturated NaHSO₃ (100 mL), and extracted with EA (200 mL). The organic layer was collected and dried over Na₂SO₄, filtered and evaporated under reduced pressure. The residue was purified on silica gel column chromatography, eluting with ethyl acetate/n-hexane (0-10%). There was tert-butyl 5-benzyloxy-4-fluoro-6-hydroxy-isoindoline-2-carboxylate (1.93 g, 5.3702 mmol, 68.93% yield) obtained as an off-white solid. LCMS: (ESI, m/z): [M−H]⁻=358.100. ¹H NMR (400 MHz, CDCl₃-d) 6 7.46-7.36 (m, 5H), 6.62 (s, 0.5H), 6.58 (s, 0.5H), 5.15 (s, 2H), 4.68 (s, 1H), 4.65 (s, 1H), 4.61 (s, 1H), 4.58 (s, 1H), 1.54 (s, 9H).

### Step g: tert-butyl 5-benzyloxy-4-fluoro-6-methoxy-isoindoline-2-carboxylate

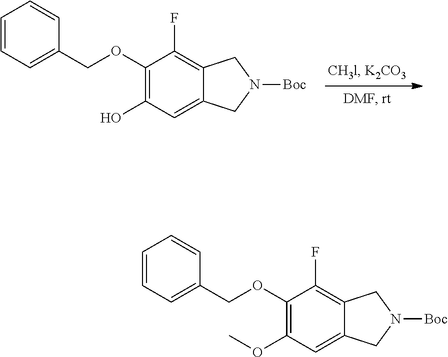 [SMILES: CC(C)(C)OC(=O)N1Cc2cc(O)c(OCc3ccccc3)c(F)c2C1 ; COc1cc2c(c(F)c1OCc1ccccc1)CN(C(=O)OC(C)(C)C)C2]

K₂CO₃ (1.18 g, 8.5380 mmol) was added to a solution of tert-butyl 5-benzyloxy-4-fluoro-6-hydroxy-isoindoline-2-carboxylate (1.9 g, 5.2867 mmol) in DMF (25 mL) at 0° C. After the mixture was stirred for 30 min at 0° C., CH₃I (1.12 g, 7.8908 mmol) was added. The reaction mixture was stirred for 3 h at 20° C. The resulting reaction mixture was diluted with water (200 mL) and extracted with EA (200 mL). The organic layer was separated and washed with brine (2×150 mL). The organic layer was collected and dried over Na₂SO₄, filtered and evaporated under reduced pressure. There was tert-butyl 5-benzyloxy-4-fluoro-6-methoxy-isoindoline-2-carboxylate (1.98 g, 5.3024 mmol, 100% yield) obtained as a colorless oil, which was directly used in the next step without purification. LCMS: (ESI, m/z): [M-tBu+ACN]- =359.150. ¹H NMR (400 MHz, CDCl₃-d) 6 7.39 (d, J=7.4 Hz, 2H), 7.31-7.21 (m, 3H), 6.51 (s, 1H), 4.99 (s, 2H), 4.55 (s, 4H), 3.77 (s, 3H), 1.44 (s, 9H).

### Step h: tert-butyl 4-fluoro-5-hydroxy-6-methoxy-isoindoline-2-carboxylate

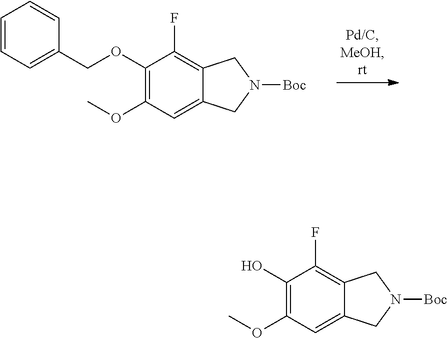 [SMILES: COc1cc2c(c(F)c1OCc1ccccc1)CN(C(=O)OC(C)(C)C)C2 ; COc1cc2c(c(F)c1O)CN(C(=O)OC(C)(C)C)C2]

10% Pd/C (1.98 g, contain 55% H₂O) was added to a solution of tert-butyl 5-benzyloxy-4-fluoro-6-methoxy-isoindoline-2-carboxylate (1.96 g, 5.2488 mmol) in MeOH (40 mL) and THF (10 mL) at 20° C. The reaction mixture was stirred for 5 h at 20° C. under H₂ atmosphere. The reaction mixture was diluted with EA (20 mL) and filtered through a celite. The filtrate was evaporated under reduced pressure. The residue was purified on silica gel column chromatography, eluting with ethyl acetate/n-hexane (0-40%). The pure fraction was concentrated and dried under reduced pressure. There was tert-butyl 4-fluoro-5-hydroxy-6-methoxy-isoindoline-2-carboxylate (1.21 g, 4.2712 mmol, 80.67% yield) obtained as a brown semi-solid. LCMS: (ESI, m/z): [M−H]⁻=282.100. ¹H NMR (400 MHz, CDCl₃-d) 6 6.58 (s, 1H), 4.67 (s, 2H), 4.63 (s, 2H), 3.92 (s, 3H), 1.53 (s, 9H).

### Step i: tert-buyl 4-chloro-5-hydroxy-6-methoxy isoindoline-2-carbox late

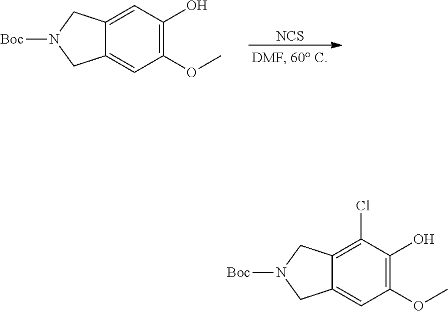 [SMILES: COc1cc2c(cc1O)CN(C(=O)OC(C)(C)C)C2 ; "NCS" ; COc1cc2c(c(Cl)c1O)CN(C(=O)OC(C)(C)C)C2]

NCS (10.92 g, 81.7776 mmol) was added to a solution of tert-butyl 5-hydroxy-6-methoxyisoindoline-2-carboxylate (20.21 g, 76.1767 mmol) in DMF (200 mL) at 0° C. The reaction mixture was stirred for 3 h at 60° C. The reaction mixture was quenched with adding of water (500 mL), extracted with EA (3×500 mL) and washed with brine (250 mL). The organics dried over Na₂SO₄, filtered and evaporated under reduced pressure. The residue was purified on silica gel column MeOH/DCM (0-2%). The pure fraction was concentrated and dried under vacuo. There was tert-butyl 4-chloro-5-hydroxy-6-methoxy-isoindoline-2-carboxylate (11.19 g, 37.3312 mmol, 49.0061% yield) obtained as a brown solid. LCMS: (ESI, m/z): [M+H-tBu+ACN]⁺=285.090.

### Step j: tert-butyl 5-(3-bromopropoxy)-4-chloro-6-methoxy-isoindoline-2-carboxylate

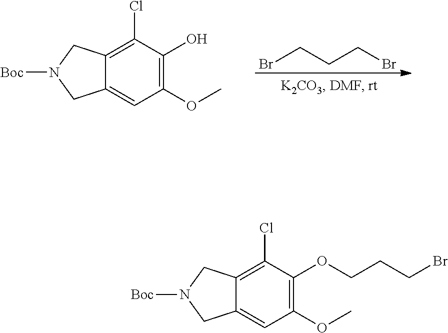 [SMILES: COc1cc2c(c(Cl)c1O)CN(C(=O)OC(C)(C)C)C2 ; BrCCCBr ; COc1cc2c(c(Cl)c1OCCCBr)CN(C(=O)OC(C)(C)C)C2]

1,3-Dibromopropane (0.498 g, 2.4667 mmol) was added to a mixture of Potassium carbonate (0.326 g, 2.3588 mmol) and tert-butyl 4-chloro-5-hydroxy-6-methoxyisoindoline-2-carboxylate (0.116 g, 386.9901 mol) in DMF (5 mL) at 20° C. under N₂ atmosphere. The reaction mixture was stirred for 3 h at 20° C. The reaction mixture was purified on silica gel column MeCN/H₂O (0.1% FA)(0-60%). The pure fraction was concentrated and dried under vacuo. There was tert-butyl 5-(3-bromopropoxy)-4-chloro-6-methoxy-isoindoline-2-carboxylate (0.099 g, 235.3082 mol, 60.8047% yield) obtained as a white solid. LCMS: (ESI, m/z): [M+H-tBu+ACN]⁺=405.050.

### Step k: tert-butyl 5-[3-(2-tert-butoxycarbonyl-4-chloro-6-methoxy-isoindolin-5-yl)oxypropoxy]-4-fluoro-6-methoxy-isoindoline-2-carboxylate

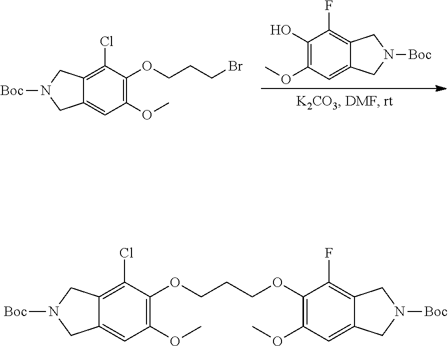

Potassium carbonate (0.115 g, 832.0942 mol) was added to a mixture of tert-butyl 4-fluoro-5-hydroxy-6-methoxyisoindoline-2-carboxylate (0.090 g, 317.6904 mol) and tert-butyl 5-(3-bromopropoxy)-4-chloro-6-methoxy-isoindoline-2-carboxylate (0.093 g, 221.0471 mol) in DMF (5 mL) at 20° C. under N₂ atmosphere. The reaction mixture was stirred for 3 h at 50° C. The reaction mixture was purified on C18 column ACN/H₂O (0.1% FA)(0-50%). The pure fraction was concentrated and dried under vacuo. There was tert-butyl 5-[3-(2-tert-butoxycarbonyl-4-chloro-6-methoxy-isoindolin-5-yl)oxypropoxy]-4-fluoro-6-methoxy-isoindoline-2-carboxylate (0.145 g, 232.7046 mol, 105.2738% yield) obtained as a white solid. LCMS: (ESI, m/z): [M+H-Boc]⁺=523.250.

¹H NMR (400 MHz, DMSO-d₆) δ 7.05 (d, J=6.2 Hz, 1H), 6.90 (d, J=5.8 Hz, 1H), 4.59 (d, J=8.4 Hz, 2H), 4.54 (d, J=7.4 Hz, 4H), 4.47 (d, J=8.9 Hz, 2H), 4.17 (t, J=6.2 Hz, 2H), 4.12 (t, J=6.2 Hz, 2H), 3.77 (t, J=5.1 Hz, 6H), 2.08-1.99 (m, 2H), 1.45 (s, 18H).

### Step l: 4-chloro-5-(3-((4-fluoro-6-methoxyisoindolin-5-yl)oxy)propoxy)-6-methoxyisoindoline dihydrochloride

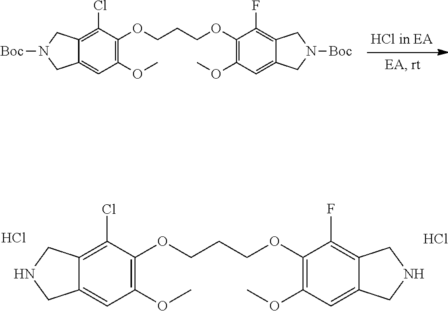 [SMILES: COc1cc2c(c(F)c1OCCCOc1c(OC)cc3c(c1Cl)CN(C(=O)OC(C)(C)C)C3)CN(C(=O)OC(C)(C)C)C2 ; Cl ; COc1cc2c(c(F)c1OCCCOc1c(OC)cc3c(c1Cl)CNC3)CNC2 ; Cl]

HCl in EA (5 mL, 20 mmol) was added to a mixture of tert- butyl 5-[3-(2-tert-butoxycarbonyl-4-chloro-6-methoxy-isoindolin-5-yl)oxypropoxy]-4-fluoro-6-methoxy-isoindoline-2-carboxylate (0.139 g, 223.0754 mol) in EA (2 mL) at 20° C. . The reaction mixture was stirred for 3 h at 20° C. The reaction mixture was evaporated under reduced pressure. There was 4-chloro-5-(3-((4-fluoro-6-methoxyisoindolin-5-yl)oxy)propoxy)-6-methoxyisoindoline dihydrochloride (0.148 g, 349.9838 mol) obtained as a brown solid, which was used directly to the next step. LCMS: (ESI, m/z): [M+H]⁺=423.140.

### Step m: 4-(5-(3-((2-(3-carboxypropanoyl)-4-chloro-6-methoxyisoindolin-5-yl)oxy)propoxy)-4-fluoro-6-methoxyisoindolin-2-yl)-4-oxobutanoic acid

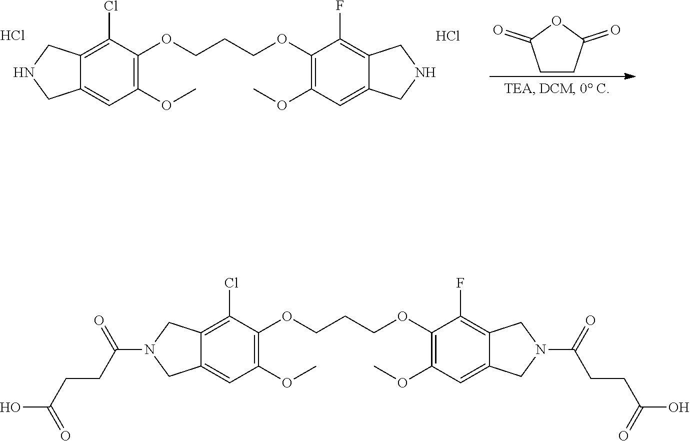 [SMILES: Cl ; COc1cc2c(c(F)c1OCCCOc1c(OC)cc3c(c1Cl)CNC3)CNC2 ; Cl ; O=C1CCC(=O)O1 ; COc1cc2c(c(F)c1OCCCOc1c(OC)cc3c(c1Cl)CN(C(=O)CCC(=O)O)C3)CN(C(=O)CCC(=O)O)C2]

Dihydrofuran-2,5-dione (0.097 g, 969.2963 mol) was added to a mixture of 4-chloro-5-(3-((4-fluoro-6-methoxyisoindolin-5-yl)oxy)propoxy)-6-methoxyisoindoline dihydrochloride (0.070 g, 165.5329 mol) and TEA (0.225 g, 2.2236 mmol) in DCM (4 mL) at 0° C. under N₂ atmosphere. The reaction mixture was stirred for 1 h at 20° C. The reaction mixture was evaporated under reduced pressure. The residue was purified on C18 column ACN/H₂O (0.1% FA)(0-50%). The pure fraction was concentrated and dried by lyophilization. There was 4-(5-(3-((2-(3-carboxypropanoyl)-4-chloro-6-methoxyisoindolin-5-yl)oxy)propoxy)-4-fluoro-6-methoxyisoindolin-2-yl)-4-oxobutanoic acid (47 mg, 75.4388 mol, 45.5733% yield) obtained as a white solid. LCMS: (ESI, m/z): [M+H]25+=623.170. ¹H NMR (400 MHz, DMSO-d₆) δ 12.07 (s, 2H), 7.08 (s, 0.5H), 7.06 (s, 0.5H), 6.93 (s, 0.5H), 6.90 (d, J=2.9 Hz, 0.5H), 4.85 (s, 1H), 4.83 (s, 1H), 4.80 (s, 1H), 4.76 (s, 1H), 4.63 (s, 1H), 4.57 (d, J=5.5 Hz, 2H), 4.51 (d, J=5.2 Hz, 1H), 4.19 (t, J=6.2 Hz, 2H), 4.13 (t, J=6.1 Hz, 2H), 3.85-3.75 (m, 6H), 2.65-2.53 (m, 6H), 2.49-2.44 (m, 2H), 2.13-1.99 (m, 2H).

### Example I-14

### 4-(5-(3-((2-((2-carboxyethyl)carbamoyl)-6-methoxyisoindolin-5-yl)oxy)propoxy)-6-methoxyisoindolin-2-yl)-4-oxobutanoic acid

### Step a: ethyl 4-(5-methoxy-6-(3-((6-methoxyisoindolin-5-yl)oxy)propoxy)isoindolin-2-yl)-4-oxobutanoate

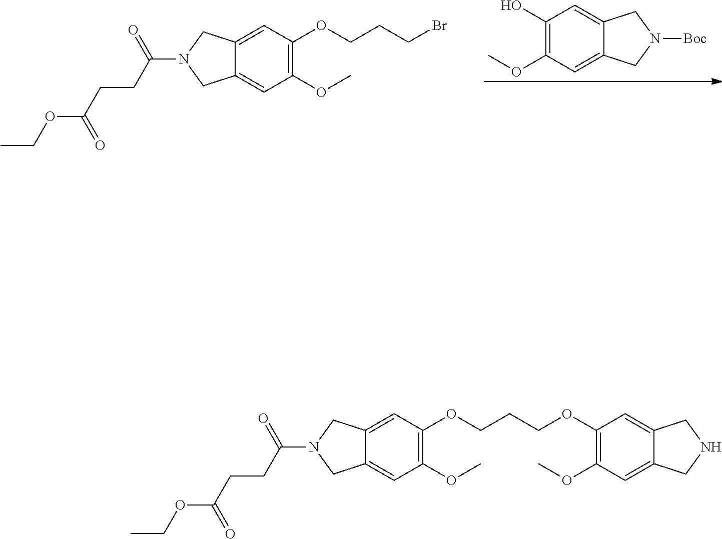 [SMILES: CCOC(=O)CCC(=O)N1Cc2cc(OC)c(OCCCBr)cc2C1 ; COc1cc2c(cc1O)CN(C(=O)OC(C)(C)C)C2 ; CCOC(=O)CCC(=O)N1Cc2cc(OC)c(OCCCOc3cc4c(cc3OC)CNC4)cc2C1]

Tert-butyl 5-hydroxy-6-methoxyisoindoline-2-carboxylate (0.149 g, 561.6195 mol) and Potassium carbonate (0.230 g, 1.6642 mmol) was added to a solution of ethyl 4-(5-(3-bromopropoxy)-6-methoxyisoindolin-2-yl)-4-oxobutanoate (0.230 g, 555.1668 mol) in N,N-Dimethylformamide (10 mL) at 20° C. The reaction mixture was stirred for 4 hours at 50° C. The reaction mixture was diluted with Ethyl acetate (100 mL), washed sequentially with water (100 mL) and brine (100 mL). The organic layer was dried over Na₂SO₄, filtered and evaporated to afford crude product. HCl (4 M) in Ethyl acetate (10 mL) was added to a solution of the crude product in Ethyl acetate (10 mL). The reaction mixture was stirred for 1 hour at 20° C. The reaction mixture was evaporated under reduced pressure. The residue was purified on C-18 column MeCN/water (0-100%). The pure fraction was concentrated and dried under vacuo. There was ethyl 4-(5-methoxy-6-(3-((6-methoxyisoindolin-5-yl)oxy)propoxy)isoindolin-2-yl)-4-oxobutanoate (0.23 g, 461.3224 mol, 83.0962% yield) obtained as a white solid. LCMS: (ESI, m/z): [M+H]⁺=499.237.

### Step b: (4-nitrophenyl) 5-[3-[2-(4-ethoxy-4-oxo-butanoyl)-6-methoxy-isoindolin-5-yl]oxypropoxy]-6-methoxy-isoindoline-2-carboxylate

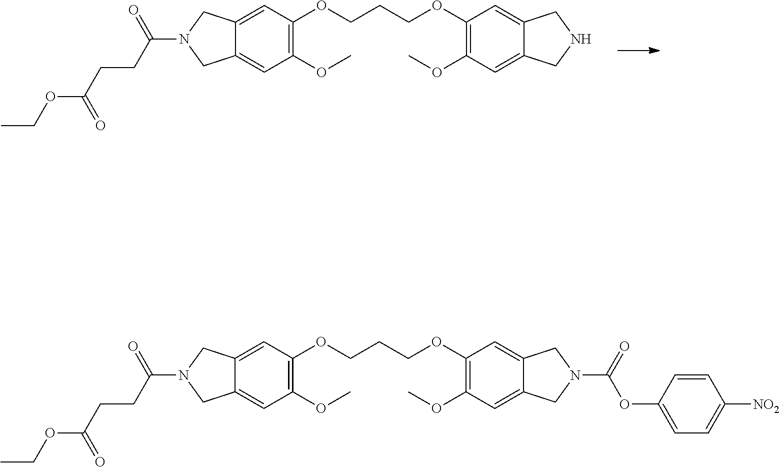 [SMILES: CCOC(=O)CCC(=O)N1Cc2cc(OC)c(OCCCOc3cc4c(cc3OC)CNC4)cc2C1 ; CCOC(=O)CCC(=O)N1Cc2cc(OC)c(OCCCOc3cc4c(cc3OC)CN(C(=O)Oc3ccc([N+](=O)[O-])cc3)C4)cc2C1]

Bis(4-nitrophenyl)carbonate (0.24 g, 788.9235 mol) and Triethylamine (0.27 g, 2.6683 mmol) was added to a solution of ethyl 4-(5-methoxy-6-(3-((6-methoxyisoindolin-5-yl)oxy)propoxy)isoindolin-2-yl)-4-oxobutanoate (0.28 g, 561.6096 mol) in N,N-Dimethylformamide (10 mL) at 20° C. The reaction mixture was stirred for 2 hours at 20° C. The reaction mixture was diluted with Ethyl acetate (100 mL), washed sequentially with water (100 mL) and brine (100 mL). The organic layer was dried over Na₂SO₄, filtered and evaporated under reduced pressure. The residue was purified on silica gel column EA/Heptane (0-100%). The pure fraction was concentrated and dried under vacuo. There was (4-nitrophenyl) 5-[3-[2-(4-ethoxy-4-oxo-butanoyl)-6-methoxy-isoindolin-5-yl]oxypropoxy]-6-methoxy-isoindoline-2-carboxylate (0.29 g, 436.9644 mol, 77.8057% yield) obtained as a white solid. LCMS: (ESI, m/z): [M+H]⁺=664.243.

### Step c: ethyl 4-(5-(3-((2-((3-ethoxy-3-oxopropyl)carbamoyl)-6-methoxyisoindolin-5-yl)oxy)propoxy)-6-methaxyisoindolin-2-yl)-4-oxabutanwate

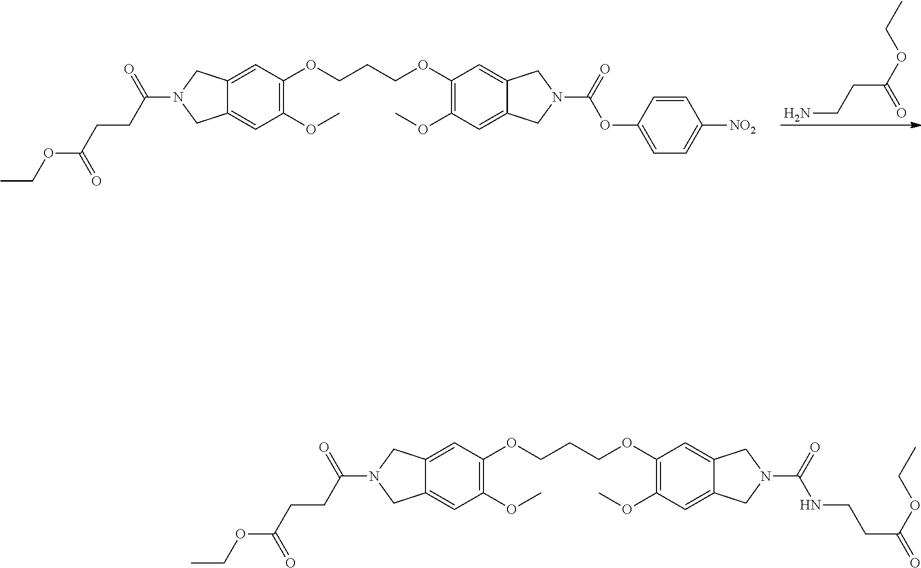

Ethyl 3-aminopropanoate hydrochloride (0.20 g, 1.3020 mmol) and N,N-Diisopropylethylamine (0.33 g, 2.5533 mmol) was added to a solution of (4-nitrophenyl) 5-[3-[2-(4-ethoxy-4-oxo-butanoyl)-6-methoxy-isoindolin-5-yl]oxypropoxy]-6-methoxy-isoindoline-2-carboxylate (0.29 g, 436.9642 mol) in N,N-Dimethylformamide (10 mL) at 20° C. The reaction mixture was stirred for 3 hours at 100° C. The reaction mixture was purified on C-18 column MeCN/water(0-100%). The pure fraction was concentrated and dried under vacuo.

There was ethyl 4-(5-(3-((2-((3-ethoxy-3-oxopropyl)carbamoyl)-6-methoxyisoindolin-5-yl)oxy)propoxy)-6-methoxyisoindolin-2-yl)-4-oxobutanoate (0.08 g, 124.6675 mol, 28.5304% yield) obtained as a yellow solid.

LCMS: (ESI, m/z): [M+H]⁺=642.295.

### Step d: 4-(5-(3-((2-((2-carboxyethyl)carbamoyl)-6-methoxyisoindolin-5-yl)oxy)propoxy)-6-methoxyisoindolin-2-yl)-4-oxobutanoic acid

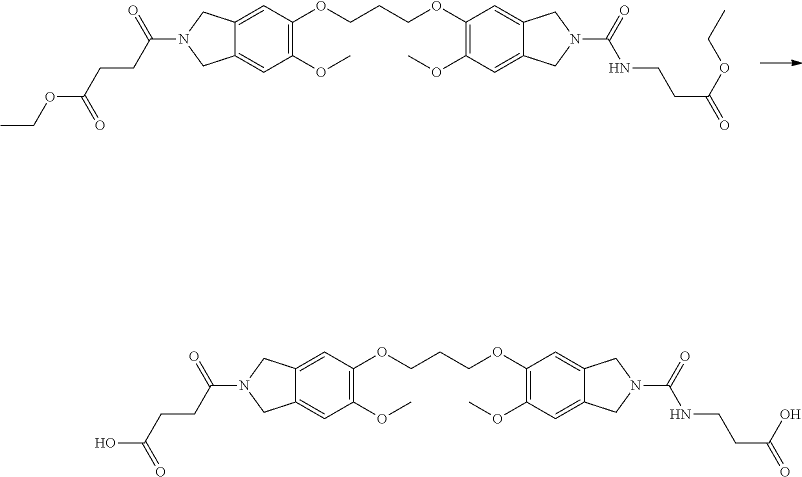 [SMILES: CCOC(=O)CCNC(=O)N1Cc2cc(OC)c(OCCCOc3cc4c(cc3OC)CN(C(=O)CCC(=O)OCC)C4)cc2C1 ; COc1cc2c(cc1OCCCOc1cc3c(cc1OC)CN(C(=O)NCCC(=O)O)C3)CN(C(=O)CCC(=O)O)C2]

LiOH (7.8371 mg, 327.2522 mol) was added to a solution of ethyl 4-(5-(3-((2-((3-ethoxy-3-oxopropyl)carbamoyl)-6-methoxyisoindolin-5-yl)oxy)propoxy)-6-methoxyisoindolin-2-yl)-4-oxobutanoate (0.07 g, 109.0841 mol) in Acetonitrile (10 mL) and Water (10 mL). The reaction mixture was stirred for 1 hours at 50° C. The reaction mixture was evaporated under reduced pressure. The residue was acidified pH=4 with HCl (1 M). The mixture was evaporated under reduced pressure. The residue was purified by Prep-HPLC. The pure fraction was concentrated and dried under vacuo. There was 4-(5-(3-((2-((2-carboxyethyl)carbamoyl)-6-methoxyisoindolin-5-yl)oxy)propoxy)-6-methoxyisoindolin-2-yl)-4-oxobutanoic acid (6.2 mg, 10.5874 mol, 9.7057% yield) obtained as a white solid. LCMS: (ESI, m/z): [M+H]⁺=586.632. ¹H NMR (400 MHz, DMSO-d₆) δ 12.14 (s, 2H), 7.03-6.88 (m, 4H), 6.36 (s, 1H), 4.70-4.75 (m, 2H), 4.53 (s, 2H), 4.46 (s, 4H), 4.10 (s, 4H), 3.74 (s, 6H), 3.23-3.30 (m, 2H), 2.57 (s, 2H), 2.40-2.50 (m, 4H), 2.15 (s, 2H).

Compounds in the following example shown in table 3 were synthesized using the above procedure with the corresponding starting materials.

TABLE 3

| | | MS: |
|---|---|---|
| EX | | (M + H)⁺ |
| No. | | & |
| I- | Structure | ¹HNMR |
| 15. | 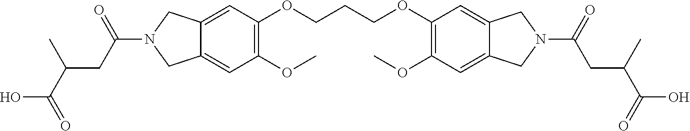 [SMILES: COc1cc2c(cc1OCCCOc1cc3c(cc1OC)CN(C(=O)CC(C)C(=O)O)C3)CN(C(=O)CC(C)C(=O)O)C2] | 599 |
| 16. | 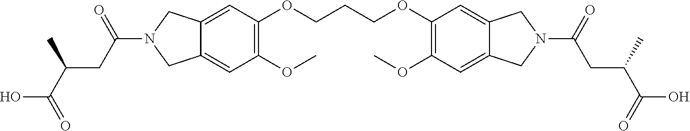 [SMILES: COc1cc2c(cc1OCCCOc1cc3c(cc1OC)CN(C(=O)C[C@H](C)C(=O)O)C3)CN(C(=O)C[C@H](C)C(=O)O)C2] | 599 |
| 17. | 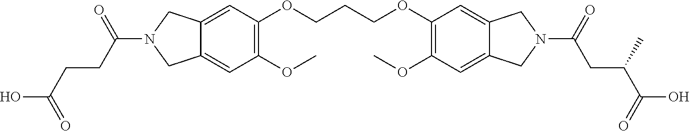 [SMILES: COc1cc2c(cc1OCCCOc1cc3c(cc1OC)CN(C(=O)C[C@H](C)C(=O)O)C3)CN(C(=O)CCC(=O)O)C2] | 585 |
| 18. | 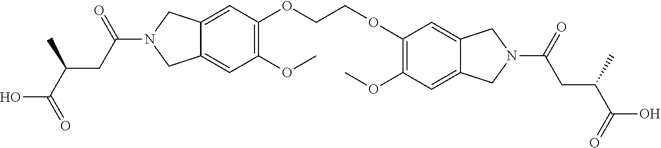 [SMILES: COc1cc2c(cc1OCCOc1cc3c(cc1OC)CN(C(=O)C[C@H](C)C(=O)O)C3)CN(C(=O)C[C@H](C)C(=O)O)C2] | 585 |
| 19. | 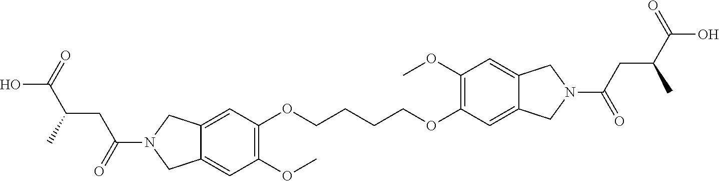 [SMILES: COc1cc2c(cc1OCCCCOc1cc3c(cc1OC)CN(C(=O)C[C@H](C)C(=O)O)C3)CN(C(=O)C[C@H](C)C(=O)O)C2] | 613 |
| 20. | 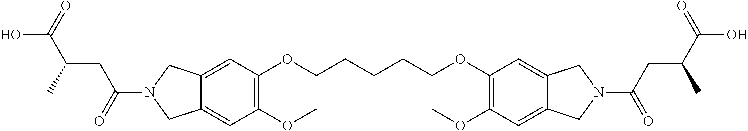 [SMILES: COc1cc2c(cc1OCCCCCOc1cc3c(cc1OC)CN(C(=O)C[C@H](C)C(=O)O)C3)CN(C(=O)C[C@H](C)C(=O)O)C2] | 627 |
| 21. | 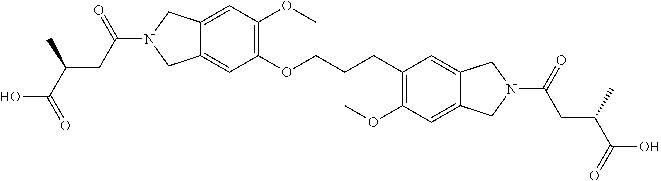 [SMILES: COc1cc2c(cc1CCCOc1cc3c(cc1OC)CN(C(=O)C[C@H](C)C(=O)O)C3)CN(C(=O)C[C@H](C)C(=O)O)C2] | 583 |
| 22. | 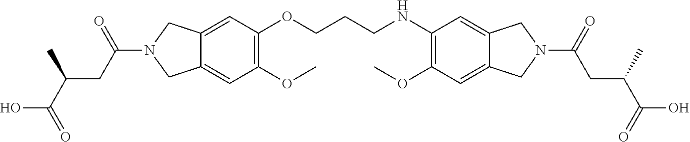 [SMILES: COc1cc2c(cc1NCCCOc1cc3c(cc1OC)CN(C(=O)C[C@H](C)C(=O)O)C3)CN(C(=O)C[C@H](C)C(=O)O)C2] | 598 |
| 23. | 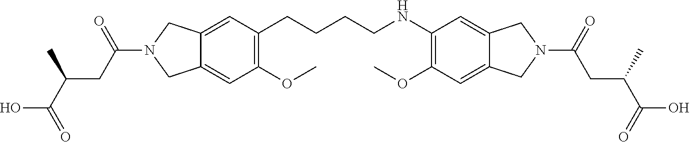 [SMILES: COc1cc2c(cc1CCCCNc1cc3c(cc1OC)CN(C(=O)C[C@H](C)C(=O)O)C3)CN(C(=O)C[C@H](C)C(=O)O)C2] | 596 |
| 24. | 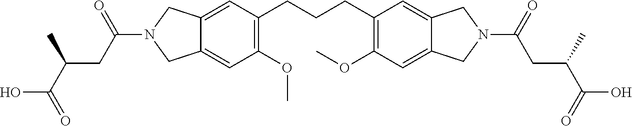 [SMILES: COc1cc2c(cc1CCCc1cc3c(cc1OC)CN(C(=O)C[C@H](C)C(=O)O)C3)CN(C(=O)C[C@H](C)C(=O)O)C2] | 567 |
| 25. | 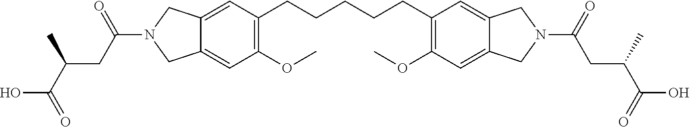 [SMILES: COc1cc2c(cc1CCCCCc1cc3c(cc1OC)CN(C(=O)C[C@H](C)C(=O)O)C3)CN(C(=O)C[C@H](C)C(=O)O)C2] | 595 |
| 26. | 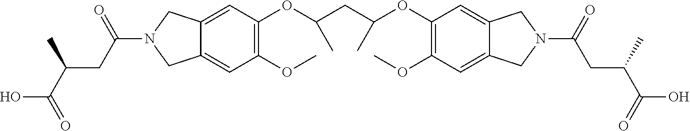 [SMILES: COc1cc2c(cc1OC(C)CC(C)Oc1cc3c(cc1OC)CN(C(=O)C[C@H](C)C(=O)O)C3)CN(C(=O)C[C@H](C)C(=O)O)C2] | 627 |
| 27. | 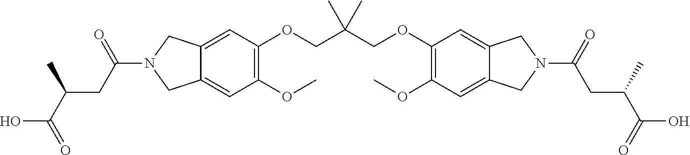 [SMILES: COc1cc2c(cc1OCC(C)(C)COc1cc3c(cc1OC)CN(C(=O)C[C@H](C)C(=O)O)C3)CN(C(=O)C[C@H](C)C(=O)O)C2] | 627 |
| 28. | 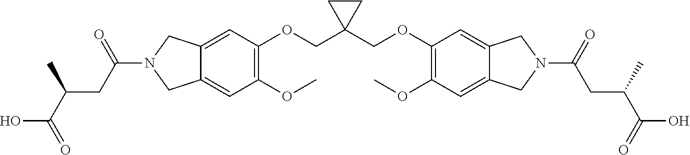 [SMILES: COc1cc2c(cc1OCC1(COc3cc4c(cc3OC)CN(C(=O)C[C@H](C)C(=O)O)C4)CC1)CN(C(=O)C[C@H](C)C(=O)O)C2] | 625 |
| 29. | 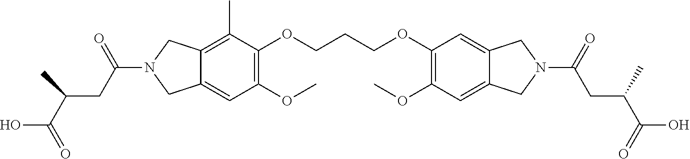 [SMILES: COc1cc2c(cc1OCCCOc1c(OC)cc3c(c1C)CN(C(=O)C[C@H](C)C(=O)O)C3)CN(C(=O)C[C@H](C)C(=O)O)C2] | 613 |
| 30. | 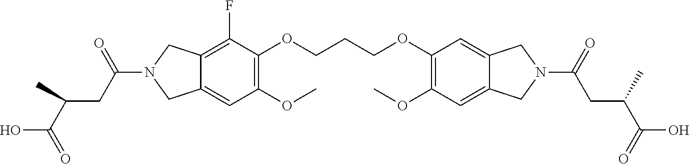 [SMILES: COc1cc2c(cc1OCCCOc1c(OC)cc3c(c1F)CN(C(=O)C[C@H](C)C(=O)O)C3)CN(C(=O)C[C@H](C)C(=O)O)C2] | 617 |
| 31. | 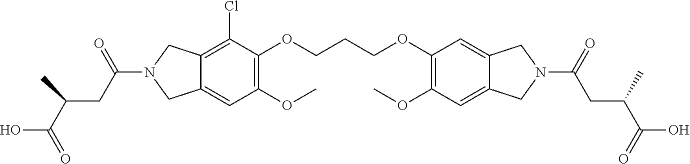 [SMILES: COc1cc2c(cc1OCCCOc1c(OC)cc3c(c1Cl)CN(C(=O)C[C@H](C)C(=O)O)C3)CN(C(=O)C[C@H](C)C(=O)O)C2] | 633 |
| 32. | 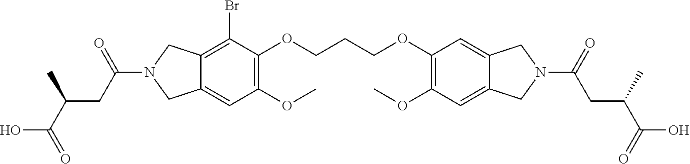 [SMILES: COc1cc2c(cc1OCCCOc1c(OC)cc3c(c1Br)CN(C(=O)C[C@H](C)C(=O)O)C3)CN(C(=O)C[C@H](C)C(=O)O)C2] | 678 |
| 33. | 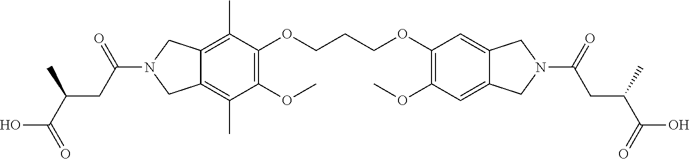 [SMILES: COc1cc2c(cc1OCCCOc1c(C)c3c(c(C)c1OC)CN(C(=O)C[C@H](C)C(=O)O)C3)CN(C(=O)C[C@H](C)C(=O)O)C2] | 627 |
| 34. | 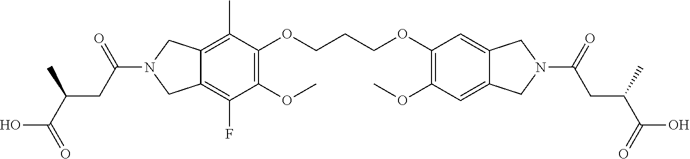 [SMILES: COc1cc2c(cc1OCCCOc1c(C)c3c(c(F)c1OC)CN(C(=O)C[C@H](C)C(=O)O)C3)CN(C(=O)C[C@H](C)C(=O)O)C2] | 631 |
| 35. | 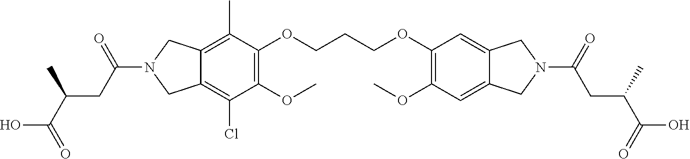 [SMILES: COc1cc2c(cc1OCCCOc1c(C)c3c(c(Cl)c1OC)CN(C(=O)C[C@H](C)C(=O)O)C3)CN(C(=O)C[C@H](C)C(=O)O)C2] | 647 |
| 36. | 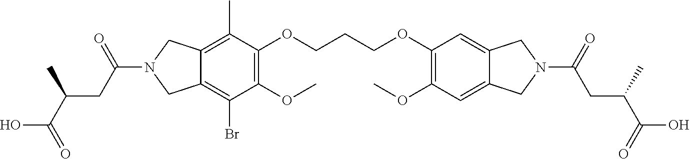 [SMILES: COc1cc2c(cc1OCCCOc1c(C)c3c(c(Br)c1OC)CN(C(=O)C[C@H](C)C(=O)O)C3)CN(C(=O)C[C@H](C)C(=O)O)C2] | 691 |
| 37. | 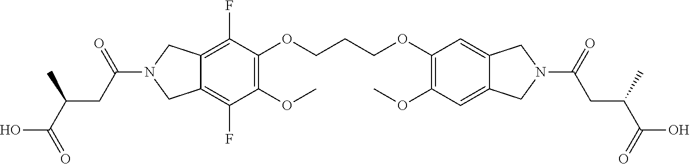 [SMILES: COc1cc2c(cc1OCCCOc1c(F)c3c(c(F)c1OC)CN(C(=O)C[C@H](C)C(=O)O)C3)CN(C(=O)C[C@H](C)C(=O)O)C2] | 635 |
| 38. | 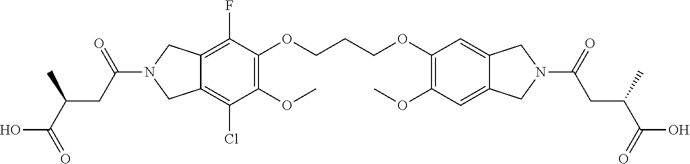 [SMILES: COc1cc2c(cc1OCCCOc1c(F)c3c(c(Cl)c1OC)CN(C(=O)C[C@H](C)C(=O)O)C3)CN(C(=O)C[C@H](C)C(=O)O)C2] | 651 |
| 39. | 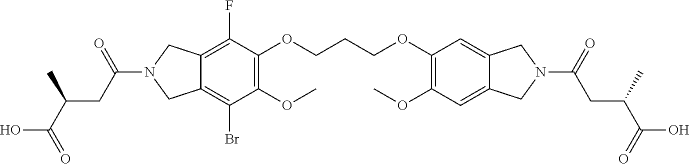 [SMILES: COc1cc2c(cc1OCCCOc1c(F)c3c(c(Br)c1OC)CN(C(=O)C[C@H](C)C(=O)O)C3)CN(C(=O)C[C@H](C)C(=O)O)C2] | 695 |
| 40. | 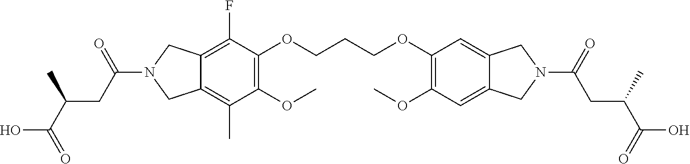 [SMILES: COc1cc2c(cc1OCCCOc1c(F)c3c(c(C)c1OC)CN(C(=O)C[C@H](C)C(=O)O)C3)CN(C(=O)C[C@H](C)C(=O)O)C2] | 631 |
| 41. | 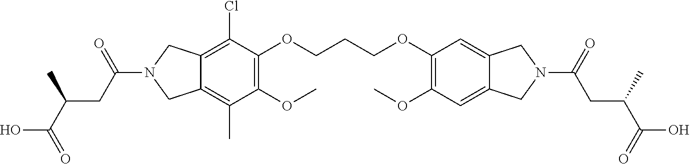 [SMILES: COc1cc2c(cc1OCCCOc1c(Cl)c3c(c(C)c1OC)CN(C(=O)C[C@H](C)C(=O)O)C3)CN(C(=O)C[C@H](C)C(=O)O)C2] | 647 |
| 42. | 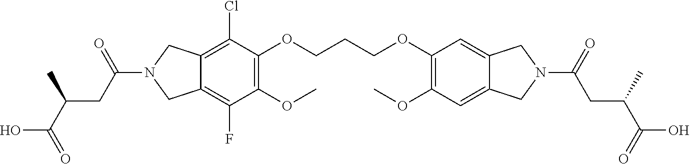 [SMILES: COc1cc2c(cc1OCCCOc1c(Cl)c3c(c(F)c1OC)CN(C(=O)C[C@H](C)C(=O)O)C3)CN(C(=O)C[C@H](C)C(=O)O)C2] | 651 |
| 43. | 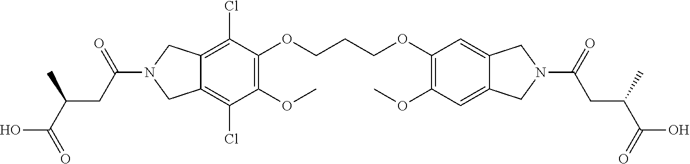 [SMILES: COc1cc2c(cc1OCCCOc1c(Cl)c3c(c(Cl)c1OC)CN(C(=O)C[C@H](C)C(=O)O)C3)CN(C(=O)C[C@H](C)C(=O)O)C2] | 667 |
| 44. | 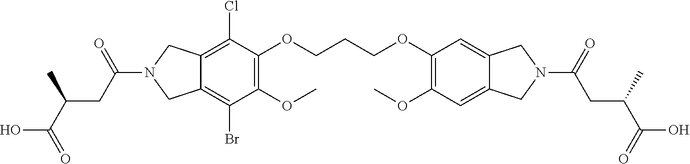 [SMILES: COc1cc2c(cc1OCCCOc1c(Cl)c3c(c(Br)c1OC)CN(C(=O)C[C@H](C)C(=O)O)C3)CN(C(=O)C[C@H](C)C(=O)O)C2] | 711 |
| 45. | 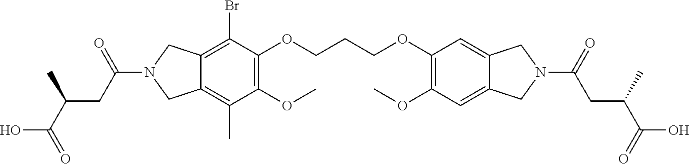 [SMILES: COc1cc2c(cc1OCCCOc1c(Br)c3c(c(C)c1OC)CN(C(=O)C[C@H](C)C(=O)O)C3)CN(C(=O)C[C@H](C)C(=O)O)C2] | 691 |
| 46. | 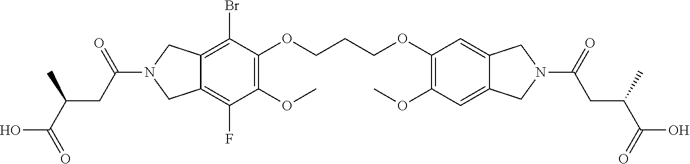 [SMILES: COc1cc2c(cc1OCCCOc1c(Br)c3c(c(F)c1OC)CN(C(=O)C[C@H](C)C(=O)O)C3)CN(C(=O)C[C@H](C)C(=O)O)C2] | 695 |
| 47. | 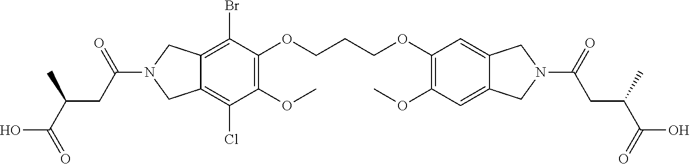 [SMILES: COc1cc2c(cc1OCCCOc1c(Br)c3c(c(Cl)c1OC)CN(C(=O)C[C@H](C)C(=O)O)C3)CN(C(=O)C[C@H](C)C(=O)O)C2] | 711 |
| 48. | 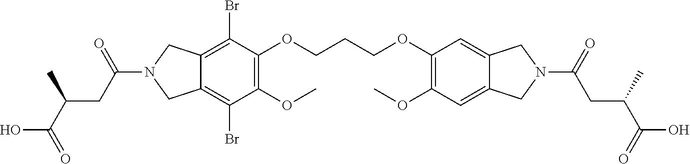 [SMILES: COc1cc2c(cc1OCCCOc1c(Br)c3c(c(Br)c1OC)CN(C(=O)C[C@H](C)C(=O)O)C3)CN(C(=O)C[C@H](C)C(=O)O)C2] | 757 |
| 49. | 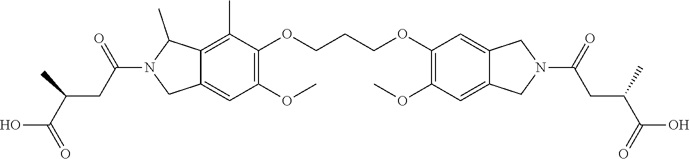 [SMILES: COc1cc2c(cc1OCCCOc1c(OC)cc3c(c1C)C(C)N(C(=O)C[C@H](C)C(=O)O)C3)CN(C(=O)C[C@H](C)C(=O)O)C2] | 627 |
| 50. | 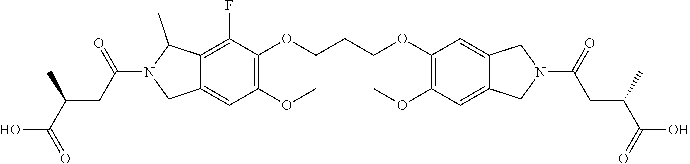 [SMILES: COc1cc2c(cc1OCCCOc1c(OC)cc3c(c1F)C(C)N(C(=O)C[C@H](C)C(=O)O)C3)CN(C(=O)C[C@H](C)C(=O)O)C2] | 631 |
| 51. | 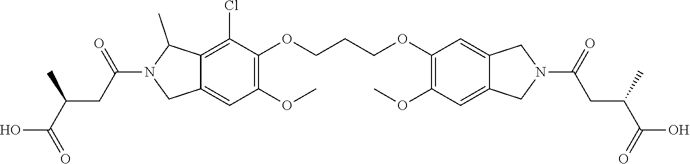 [SMILES: COc1cc2c(cc1OCCCOc1c(OC)cc3c(c1Cl)C(C)N(C(=O)C[C@H](C)C(=O)O)C3)CN(C(=O)C[C@H](C)C(=O)O)C2] | 647 |
| 52. | 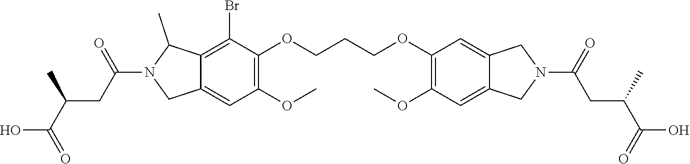 [SMILES: COc1cc2c(cc1OCCCOc1c(OC)cc3c(c1Br)C(C)N(C(=O)C[C@H](C)C(=O)O)C3)CN(C(=O)C[C@H](C)C(=O)O)C2] | 691 |
| 53. | 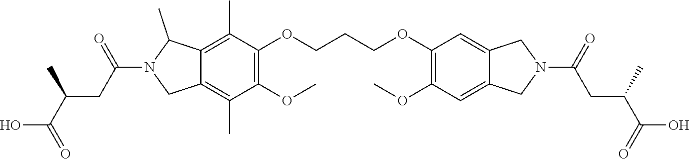 [SMILES: COc1cc2c(cc1OCCCOc1c(C)c3c(c(C)c1OC)CN(C(=O)C[C@H](C)C(=O)O)C3C)CN(C(=O)C[C@H](C)C(=O)O)C2] | 641 |
| 54. | 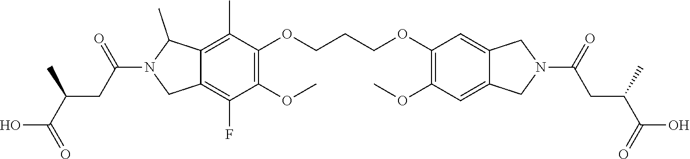 [SMILES: COc1cc2c(cc1OCCCOc1c(C)c3c(c(F)c1OC)CN(C(=O)C[C@H](C)C(=O)O)C3C)CN(C(=O)C[C@H](C)C(=O)O)C2] | 645 |
| 55. | 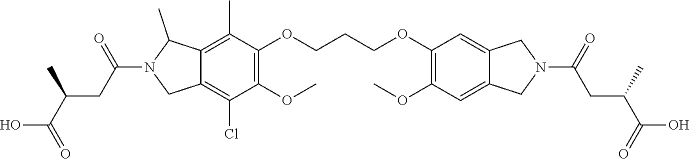 [SMILES: COc1cc2c(cc1OCCCOc1c(C)c3c(c(Cl)c1OC)CN(C(=O)C[C@H](C)C(=O)O)C3C)CN(C(=O)C[C@H](C)C(=O)O)C2] | 661 |
| 56. | 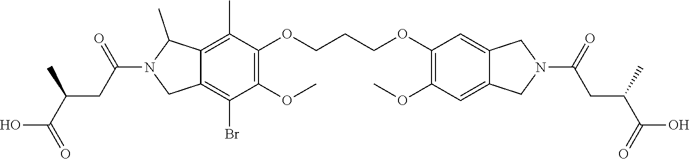 [SMILES: COc1cc2c(cc1OCCCOc1c(C)c3c(c(Br)c1OC)CN(C(=O)C[C@H](C)C(=O)O)C3C)CN(C(=O)C[C@H](C)C(=O)O)C2] | 705 |
| 57. | 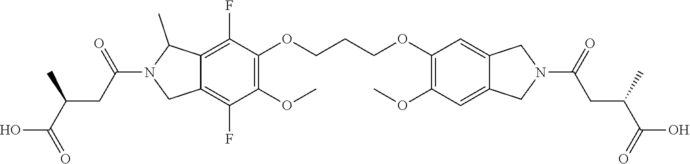 [SMILES: COc1cc2c(cc1OCCCOc1c(F)c3c(c(F)c1OC)CN(C(=O)C[C@H](C)C(=O)O)C3C)CN(C(=O)C[C@H](C)C(=O)O)C2] | 649 |
| 58. | 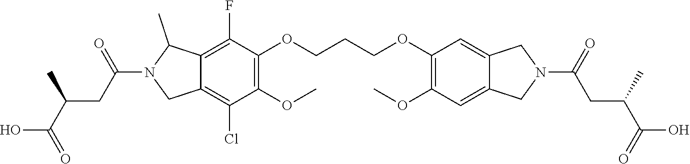 [SMILES: COc1cc2c(cc1OCCCOc1c(F)c3c(c(Cl)c1OC)CN(C(=O)C[C@H](C)C(=O)O)C3C)CN(C(=O)C[C@H](C)C(=O)O)C2] | 665 |
| 59. | 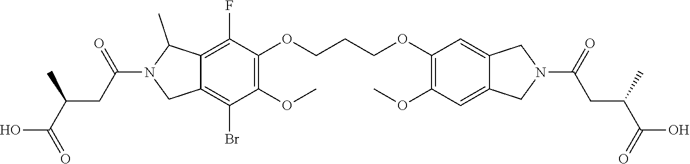 [SMILES: COc1cc2c(cc1OCCCOc1c(F)c3c(c(Br)c1OC)CN(C(=O)C[C@H](C)C(=O)O)C3C)CN(C(=O)C[C@H](C)C(=O)O)C2] | 709 |
| 60. | 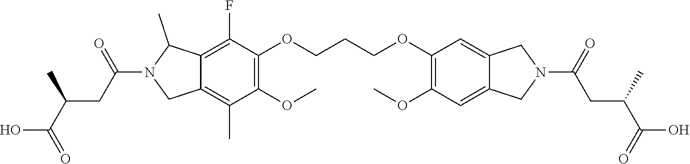 [SMILES: COc1cc2c(cc1OCCCOc1c(F)c3c(c(C)c1OC)CN(C(=O)C[C@H](C)C(=O)O)C3C)CN(C(=O)C[C@H](C)C(=O)O)C2] | 645 |
| 61. | 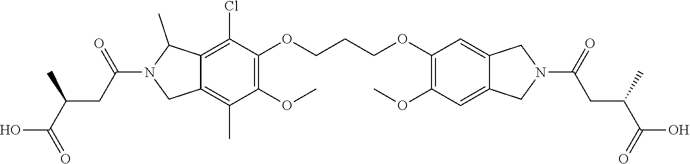 [SMILES: COc1cc2c(cc1OCCCOc1c(Cl)c3c(c(C)c1OC)CN(C(=O)C[C@H](C)C(=O)O)C3C)CN(C(=O)C[C@H](C)C(=O)O)C2] | 661 |
| 62. | 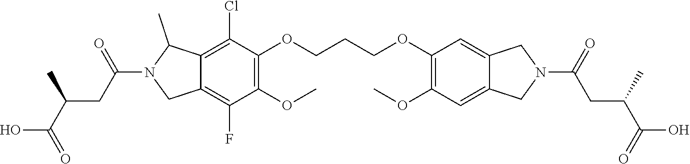 [SMILES: COc1cc2c(cc1OCCCOc1c(Cl)c3c(c(F)c1OC)CN(C(=O)C[C@H](C)C(=O)O)C3C)CN(C(=O)C[C@H](C)C(=O)O)C2] | 665 |
| 63. | 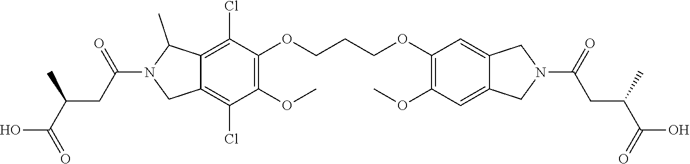 [SMILES: COc1cc2c(cc1OCCCOc1c(Cl)c3c(c(Cl)c1OC)CN(C(=O)C[C@H](C)C(=O)O)C3C)CN(C(=O)C[C@H](C)C(=O)O)C2] | 681 |
| 64. | 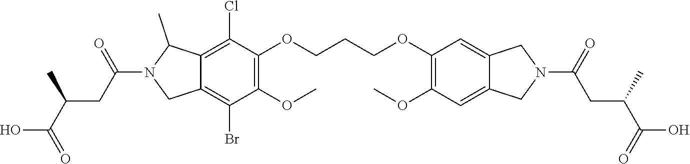 [SMILES: COc1cc2c(cc1OCCCOc1c(Cl)c3c(c(Br)c1OC)CN(C(=O)C[C@H](C)C(=O)O)C3C)CN(C(=O)C[C@H](C)C(=O)O)C2] | 725 |
| 65. | 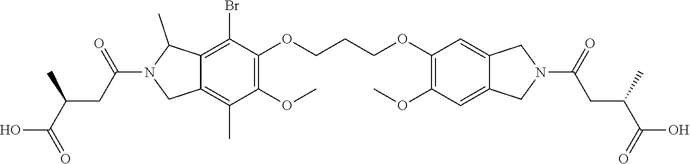 [SMILES: COc1cc2c(cc1OCCCOc1c(Br)c3c(c(C)c1OC)CN(C(=O)C[C@H](C)C(=O)O)C3C)CN(C(=O)C[C@H](C)C(=O)O)C2] | 705 |
| 66. | 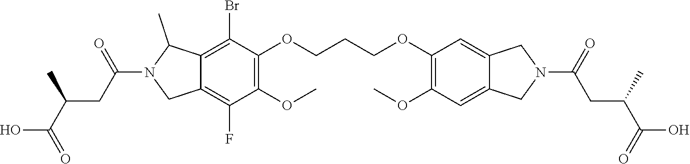 [SMILES: COc1cc2c(cc1OCCCOc1c(Br)c3c(c(F)c1OC)CN(C(=O)C[C@H](C)C(=O)O)C3C)CN(C(=O)C[C@H](C)C(=O)O)C2] | 709 |
| 67. | 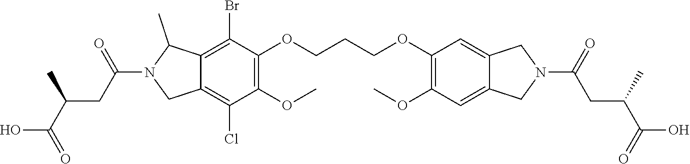 [SMILES: COc1cc2c(cc1OCCCOc1c(Br)c3c(c(Cl)c1OC)CN(C(=O)C[C@H](C)C(=O)O)C3C)CN(C(=O)C[C@H](C)C(=O)O)C2] | 725 |
| 68. | 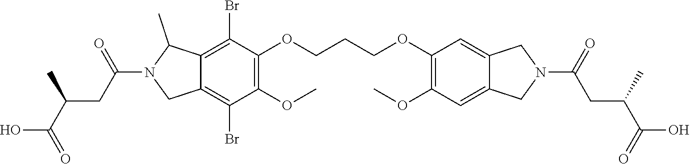 [SMILES: COc1cc2c(cc1OCCCOc1c(Br)c3c(c(Br)c1OC)CN(C(=O)C[C@H](C)C(=O)O)C3C)CN(C(=O)C[C@H](C)C(=O)O)C2] | 771 |
| 69. | 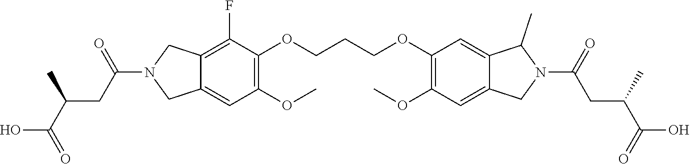 [SMILES: COc1cc2c(cc1OCCCOc1c(OC)cc3c(c1F)CN(C(=O)C[C@H](C)C(=O)O)C3)C(C)N(C(=O)C[C@H](C)C(=O)O)C2] | 631 |
| 70. | 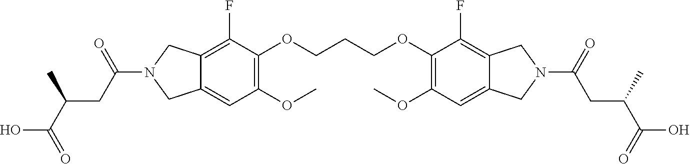 [SMILES: COc1cc2c(c(F)c1OCCCOc1c(OC)cc3c(c1F)CN(C(=O)C[C@H](C)C(=O)O)C3)CN(C(=O)C[C@H](C)C(=O)O)C2] | 635 |
| 71. | 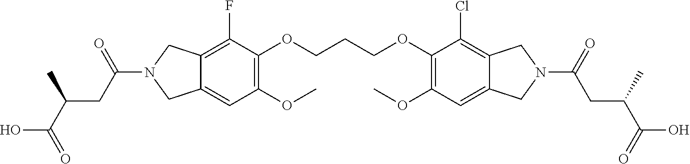 [SMILES: COc1cc2c(c(F)c1OCCCOc1c(OC)cc3c(c1Cl)CN(C(=O)C[C@H](C)C(=O)O)C3)CN(C(=O)C[C@H](C)C(=O)O)C2] | 651 |
| 72. | 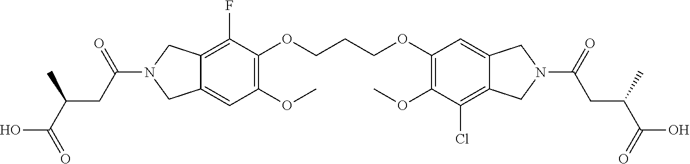 [SMILES: COc1cc2c(c(F)c1OCCCOc1cc3c(c(Cl)c1OC)CN(C(=O)C[C@H](C)C(=O)O)C3)CN(C(=O)C[C@H](C)C(=O)O)C2] | 651 |
| 73. | 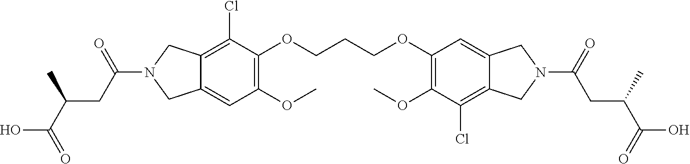 [SMILES: COc1cc2c(c(Cl)c1OCCCOc1cc3c(c(Cl)c1OC)CN(C(=O)C[C@H](C)C(=O)O)C3)CN(C(=O)C[C@H](C)C(=O)O)C2] | 667 |
| 74. | 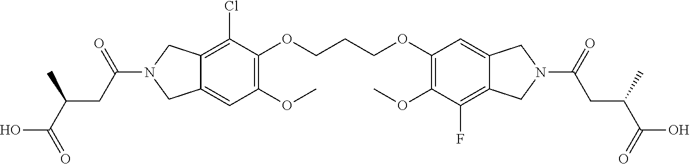 [SMILES: COc1cc2c(c(Cl)c1OCCCOc1cc3c(c(F)c1OC)CN(C(=O)C[C@H](C)C(=O)O)C3)CN(C(=O)C[C@H](C)C(=O)O)C2] | 651 |
| 75. | 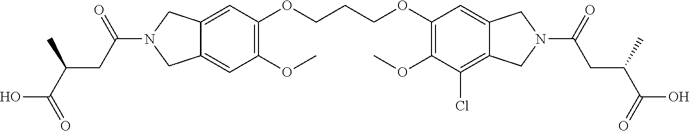 [SMILES: COc1cc2c(cc1OCCCOc1cc3c(c(Cl)c1OC)CN(C(=O)C[C@H](C)C(=O)O)C3)CN(C(=O)C[C@H](C)C(=O)O)C2] | 633 |
| 76. | 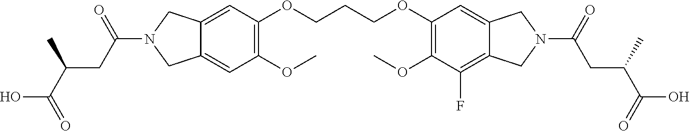 [SMILES: COc1cc2c(cc1OCCCOc1cc3c(c(F)c1OC)CN(C(=O)C[C@H](C)C(=O)O)C3)CN(C(=O)C[C@H](C)C(=O)O)C2] | 617 |
| 77. | 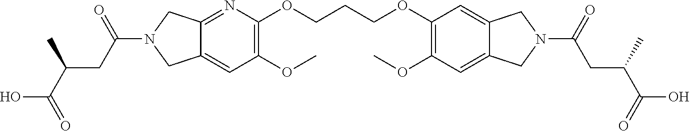 [SMILES: COc1cc2c(cc1OCCCOc1nc3c(cc1OC)CN(C(=O)C[C@H](C)C(=O)O)C3)CN(C(=O)C[C@H](C)C(=O)O)C2] | 600 |
| 78. | 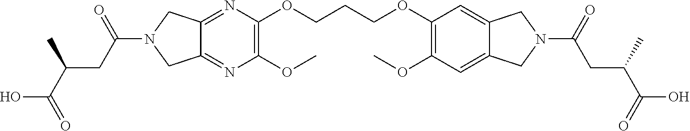 [SMILES: COc1cc2c(cc1OCCCOc1nc3c(nc1OC)CN(C(=O)C[C@H](C)C(=O)O)C3)CN(C(=O)C[C@H](C)C(=O)O)C2] | 601 |
| 79. | 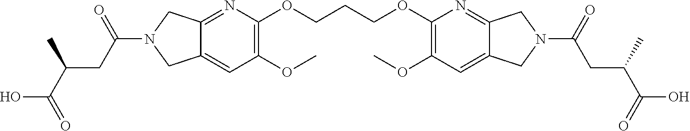 [SMILES: COc1cc2c(nc1OCCCOc1nc3c(cc1OC)CN(C(=O)C[C@H](C)C(=O)O)C3)CN(C(=O)C[C@H](C)C(=O)O)C2] | 601 |
| 80. | 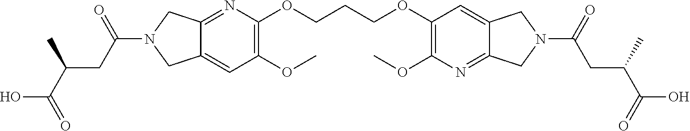 [SMILES: COc1cc2c(nc1OCCCOc1cc3c(nc1OC)CN(C(=O)C[C@H](C)C(=O)O)C3)CN(C(=O)C[C@H](C)C(=O)O)C2] | 601 |
| 81. | 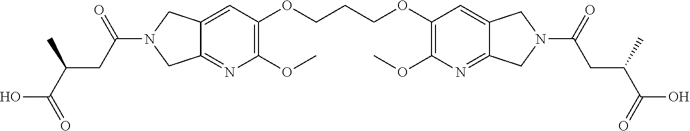 [SMILES: COc1nc2c(cc1OCCCOc1cc3c(nc1OC)CN(C(=O)C[C@H](C)C(=O)O)C3)CN(C(=O)C[C@H](C)C(=O)O)C2] | 601 |
| 82. | 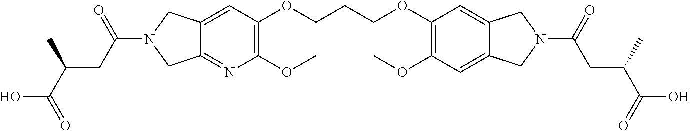 [SMILES: COc1cc2c(cc1OCCCOc1cc3c(nc1OC)CN(C(=O)C[C@H](C)C(=O)O)C3)CN(C(=O)C[C@H](C)C(=O)O)C2] | 600 |
| 83. | 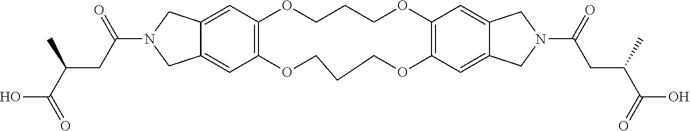 [SMILES: C[C@@H](CC(=O)N1Cc2cc3c(cc2C1)OCCCOc1cc2c(cc1OCCCO3)CN(C(=O)C[C@H](C)C(=O)O)C2)C(=O)O] | 611 |
| 84. | 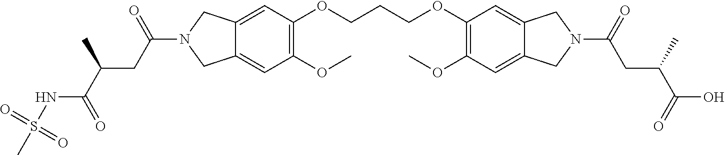 [SMILES: COc1cc2c(cc1OCCCOc1cc3c(cc1OC)CN(C(=O)C[C@H](C)C(=O)NS(C)(=O)=O)C3)CN(C(=O)C[C@H](C)C(=O)O)C2] | 676 |
| 85. | 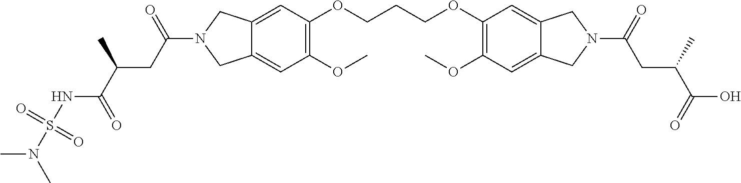 [SMILES: COc1cc2c(cc1OCCCOc1cc3c(cc1OC)CN(C(=O)C[C@H](C)C(=O)NS(=O)(=O)N(C)C)C3)CN(C(=O)C[C@H](C)C(=O)O)C2] | 705 |
| 86. | 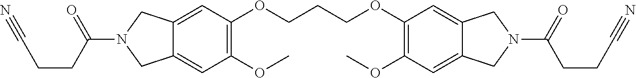 [SMILES: COc1cc2c(cc1OCCCOc1cc3c(cc1OC)CN(C(=O)CCC#N)C3)CN(C(=O)CCC#N)C2] | 533 |
| 87. | 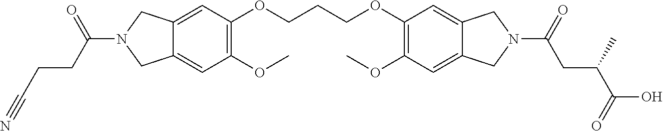 [SMILES: COc1cc2c(cc1OCCCOc1cc3c(cc1OC)CN(C(=O)C[C@H](C)C(=O)O)C3)CN(C(=O)CCC#N)C2] | 566 |
| 88. | 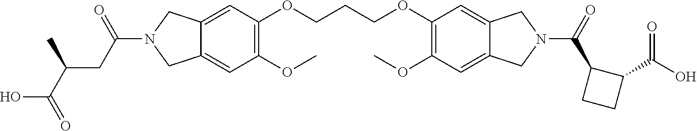 [SMILES: COc1cc2c(cc1OCCCOc1cc3c(cc1OC)CN(C(=O)[C@@H]1CC[C@H]1C(=O)O)C3)CN(C(=O)C[C@H](C)C(=O)O)C2] | 611 |
| 89. | 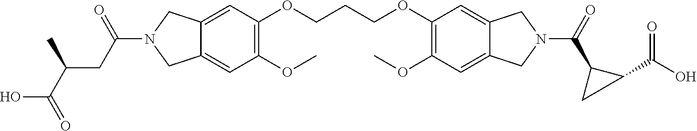 [SMILES: COc1cc2c(cc1OCCCOc1cc3c(cc1OC)CN(C(=O)[C@@H]1C[C@H]1C(=O)O)C3)CN(C(=O)C[C@H](C)C(=O)O)C2] | 597 |
| 90. | 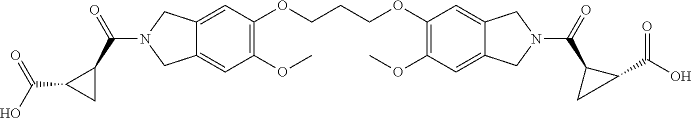 [SMILES: COc1cc2c(cc1OCCCOc1cc3c(cc1OC)CN(C(=O)[C@@H]1C[C@H]1C(=O)O)C3)CN(C(=O)[C@H]1C[C@@H]1C(=O)O)C2] | 595 |
| 91. | 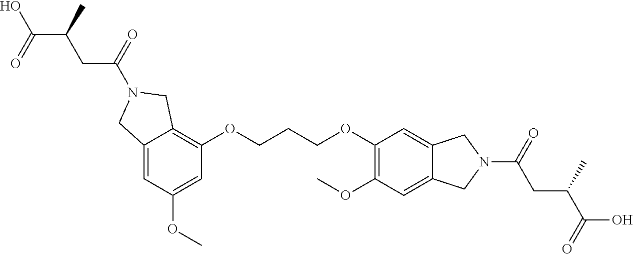 [SMILES: COc1cc2c(c(OCCCOc3cc4c(cc3OC)CN(C(=O)C[C@H](C)C(=O)O)C4)c1)CN(C(=O)C[C@H](C)C(=O)O)C2] | 599 |
| 92. | 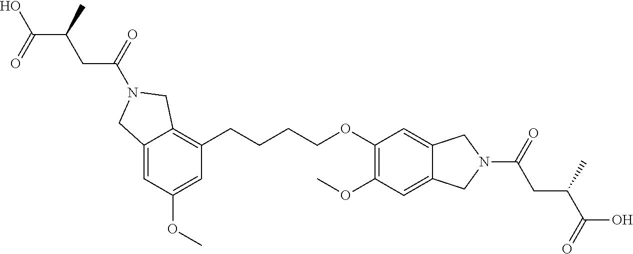 [SMILES: COc1cc(CCCCOc2cc3c(cc2OC)CN(C(=O)C[C@H](C)C(=O)O)C3)c2c(c1)CN(C(=O)C[C@H](C)C(=O)O)C2] | 597 |
| 93. | 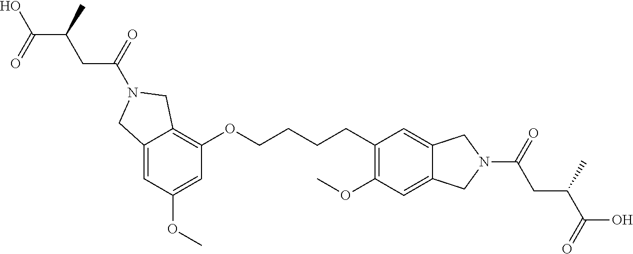 [SMILES: COc1cc2c(c(OCCCCc3cc4c(cc3OC)CN(C(=O)C[C@H](C)C(=O)O)C4)c1)CN(C(=O)C[C@H](C)C(=O)O)C2] | 597 |
| 94. | 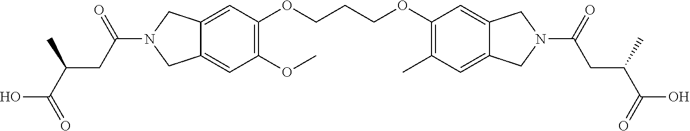 [SMILES: COc1cc2c(cc1OCCCOc1cc3c(cc1C)CN(C(=O)C[C@H](C)C(=O)O)C3)CN(C(=O)C[C@H](C)C(=O)O)C2] | 583 |
| 95. | 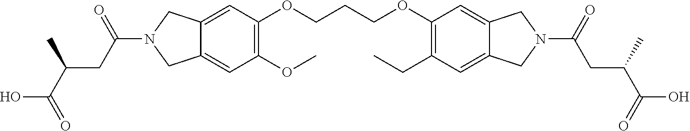 [SMILES: CCc1cc2c(cc1OCCCOc1cc3c(cc1OC)CN(C(=O)C[C@H](C)C(=O)O)C3)CN(C(=O)C[C@H](C)C(=O)O)C2] | 597 |
| 96. | 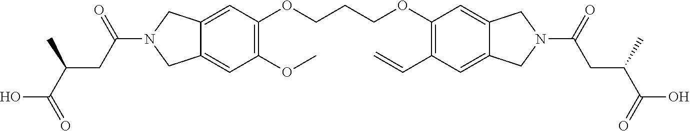 [SMILES: C=Cc1cc2c(cc1OCCCOc1cc3c(cc1OC)CN(C(=O)C[C@H](C)C(=O)O)C3)CN(C(=O)C[C@H](C)C(=O)O)C2] | 595 |
| 97. | 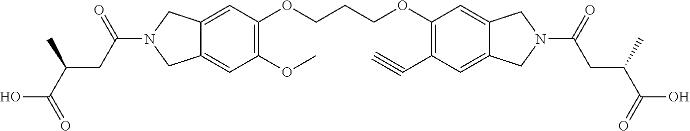 [SMILES: C#Cc1cc2c(cc1OCCCOc1cc3c(cc1OC)CN(C(=O)C[C@H](C)C(=O)O)C3)CN(C(=O)C[C@H](C)C(=O)O)C2] | 593 |
| 98. | 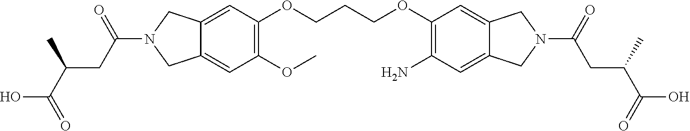 [SMILES: COc1cc2c(cc1OCCCOc1cc3c(cc1N)CN(C(=O)C[C@H](C)C(=O)O)C3)CN(C(=O)C[C@H](C)C(=O)O)C2] | 584 |
| 99. | 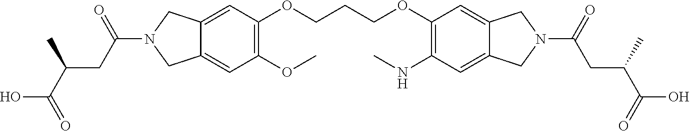 [SMILES: CNc1cc2c(cc1OCCCOc1cc3c(cc1OC)CN(C(=O)C[C@H](C)C(=O)O)C3)CN(C(=O)C[C@H](C)C(=O)O)C2] | 598 |
| 100. | 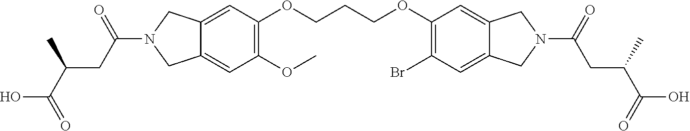 [SMILES: COc1cc2c(cc1OCCCOc1cc3c(cc1Br)CN(C(=O)C[C@H](C)C(=O)O)C3)CN(C(=O)C[C@H](C)C(=O)O)C2] | 647 |
| 101. | 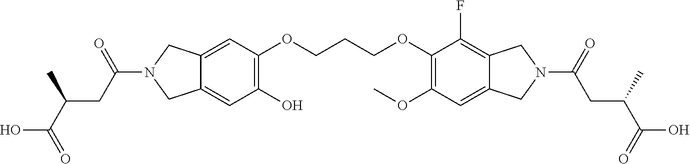 [SMILES: COc1cc2c(c(F)c1OCCCOc1cc3c(cc1O)CN(C(=O)C[C@H](C)C(=O)O)C3)CN(C(=O)C[C@H](C)C(=O)O)C2] | 603 |
| 102. | 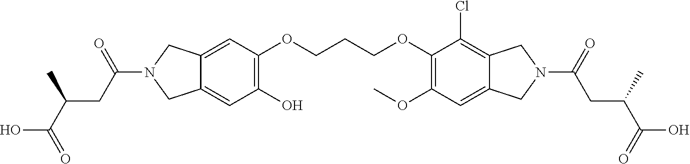 [SMILES: COc1cc2c(c(Cl)c1OCCCOc1cc3c(cc1O)CN(C(=O)C[C@H](C)C(=O)O)C3)CN(C(=O)C[C@H](C)C(=O)O)C2] | 619 |
| 103. | 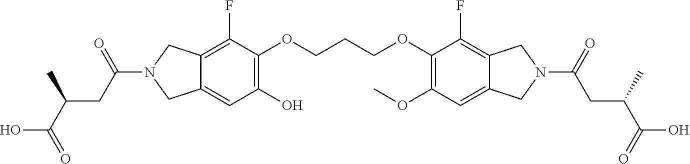 [SMILES: COc1cc2c(c(F)c1OCCCOc1c(O)cc3c(c1F)CN(C(=O)C[C@H](C)C(=O)O)C3)CN(C(=O)C[C@H](C)C(=O)O)C2] | 621 |
| 104. | 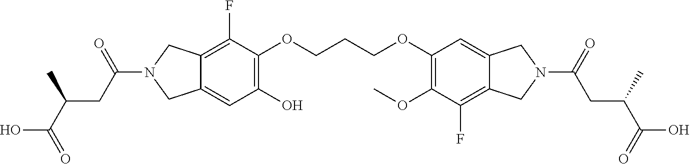 [SMILES: COc1c(OCCCOc2c(O)cc3c(c2F)CN(C(=O)C[C@H](C)C(=O)O)C3)cc2c(c1F)CN(C(=O)C[C@H](C)C(=O)O)C2] | 621 |
| 105. | 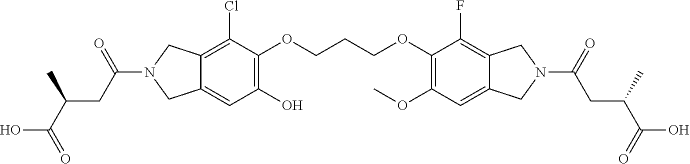 [SMILES: COc1cc2c(c(F)c1OCCCOc1c(O)cc3c(c1Cl)CN(C(=O)C[C@H](C)C(=O)O)C3)CN(C(=O)C[C@H](C)C(=O)O)C2] | 637 |
| 106. | 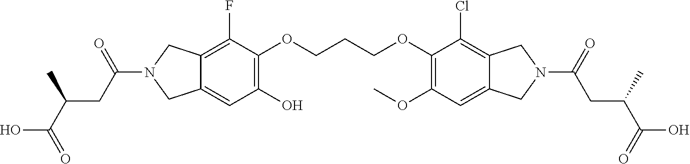 [SMILES: COc1cc2c(c(Cl)c1OCCCOc1c(O)cc3c(c1F)CN(C(=O)C[C@H](C)C(=O)O)C3)CN(C(=O)C[C@H](C)C(=O)O)C2] | 637 |
| 107. | 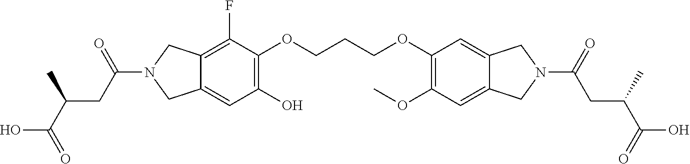 [SMILES: COc1cc2c(cc1OCCCOc1c(O)cc3c(c1F)CN(C(=O)C[C@H](C)C(=O)O)C3)CN(C(=O)C[C@H](C)C(=O)O)C2] | 603 |
| 108. | 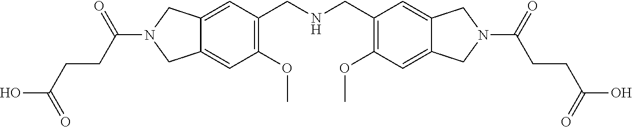 [SMILES: COc1cc2c(cc1CNCc1cc3c(cc1OC)CN(C(=O)CCC(=O)O)C3)CN(C(=O)CCC(=O)O)C2] | 540 |
| 109. | 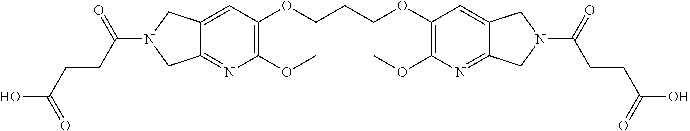 [SMILES: COc1nc2c(cc1OCCCOc1cc3c(nc1OC)CN(C(=O)CCC(=O)O)C3)CN(C(=O)CCC(=O)O)C2] | 573 |
| 110. | 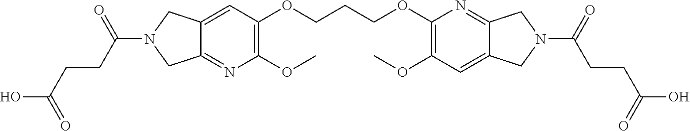 [SMILES: COc1cc2c(nc1OCCCOc1cc3c(nc1OC)CN(C(=O)CCC(=O)O)C3)CN(C(=O)CCC(=O)O)C2] | 573 |
| 111. | 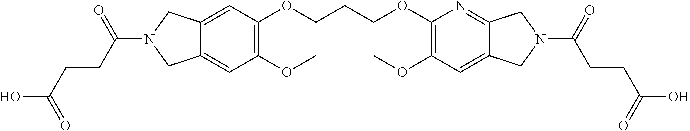 [SMILES: COc1cc2c(cc1OCCCOc1nc3c(cc1OC)CN(C(=O)CCC(=O)O)C3)CN(C(=O)CCC(=O)O)C2] | 572 |
| 112. | 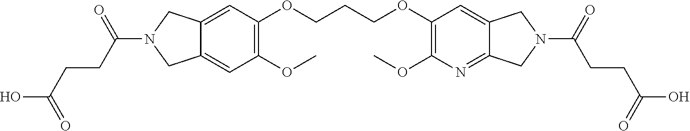 [SMILES: COc1cc2c(cc1OCCCOc1cc3c(nc1OC)CN(C(=O)CCC(=O)O)C3)CN(C(=O)CCC(=O)O)C2] | 572 |
| 113. | 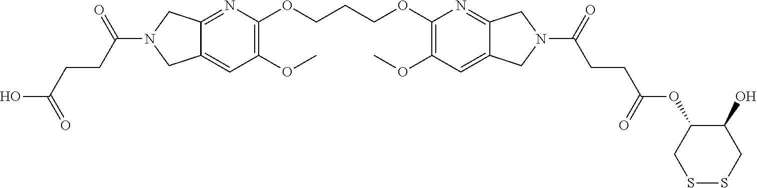 [SMILES: COc1cc2c(nc1OCCCOc1nc3c(cc1OC)CN(C(=O)CCC(=O)O[C@H]1CSSC[C@@H]1O)C3)CN(C(=O)CCC(=O)O)C2] | 707 |
| 114. | 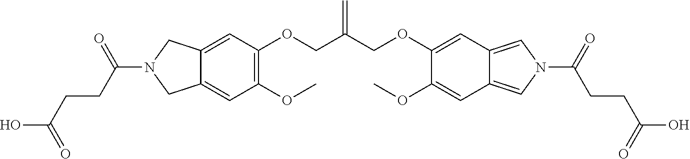 [SMILES: C=C(COc1cc2c(cc1OC)CN(C(=O)CCC(=O)O)C2)COc1cc2cn(C(=O)CCC(=O)O)cc2cc1OC] | 581 |
| 115. | 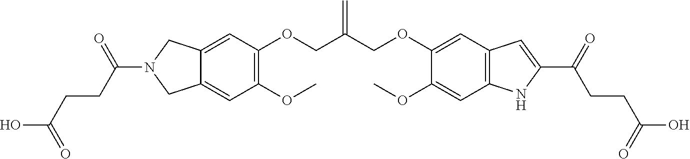 [SMILES: C=C(COc1cc2c(cc1OC)CN(C(=O)CCC(=O)O)C2)COc1cc2cc(C(=O)CCC(=O)O)[nH]c2cc1OC] | 581 |
| 116. | 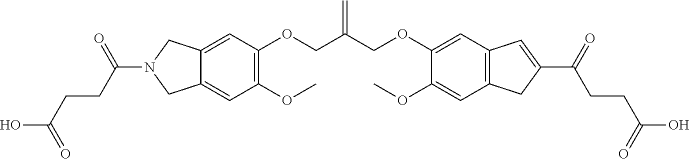 [SMILES: C=C(COc1cc2c(cc1OC)CC(C(=O)CCC(=O)O)=C2)COc1cc2c(cc1OC)CN(C(=O)CCC(=O)O)C2] | 580 |
| 117. | 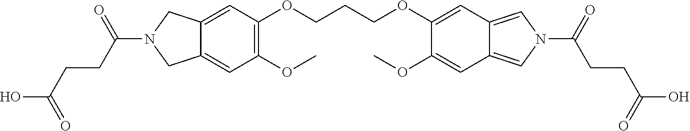 [SMILES: COc1cc2c(cc1OCCCOc1cc3cn(C(=O)CCC(=O)O)cc3cc1OC)CN(C(=O)CCC(=O)O)C2] | 569 |
| 118. | 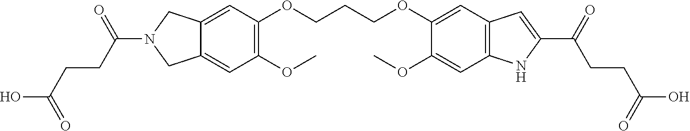 [SMILES: COc1cc2c(cc1OCCCOc1cc3cc(C(=O)CCC(=O)O)[nH]c3cc1OC)CN(C(=O)CCC(=O)O)C2] | 569 |
| 119. | 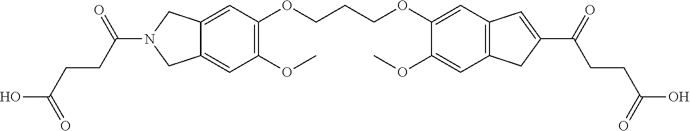 [SMILES: COc1cc2c(cc1OCCCOc1cc3c(cc1OC)CN(C(=O)CCC(=O)O)C3)C=C(C(=O)CCC(=O)O)C2] | 568 |
| 120. | 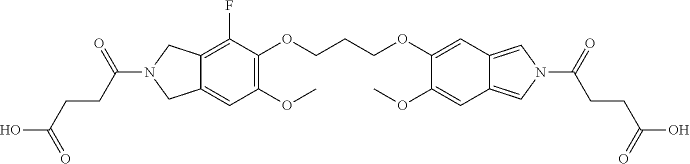 [SMILES: COc1cc2cn(C(=O)CCC(=O)O)cc2cc1OCCCOc1c(OC)cc2c(c1F)CN(C(=O)CCC(=O)O)C2] | 587 |
| 121. | 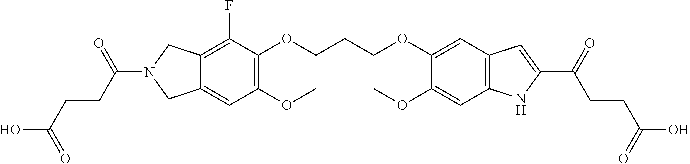 [SMILES: COc1cc2[nH]c(C(=O)CCC(=O)O)cc2cc1OCCCOc1c(OC)cc2c(c1F)CN(C(=O)CCC(=O)O)C2] | 587 |
| 122. | 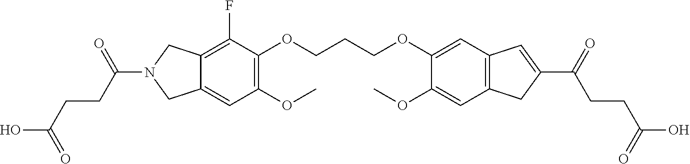 [SMILES: COc1cc2c(cc1OCCCOc1c(OC)cc3c(c1F)CN(C(=O)CCC(=O)O)C3)C=C(C(=O)CCC(=O)O)C2] | 586 |
| 123. | 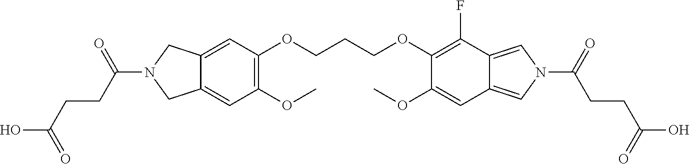 [SMILES: COc1cc2c(cc1OCCCOc1c(OC)cc3cn(C(=O)CCC(=O)O)cc3c1F)CN(C(=O)CCC(=O)O)C2] | 587 |
| 124. | 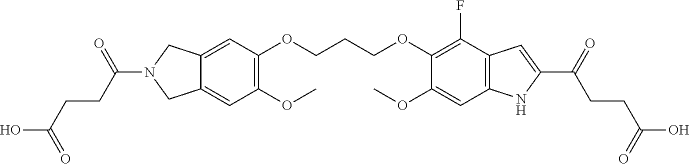 [SMILES: COc1cc2c(cc1OCCCOc1c(OC)cc3[nH]c(C(=O)CCC(=O)O)cc3c1F)CN(C(=O)CCC(=O)O)C2] | 587 |
| 125. | 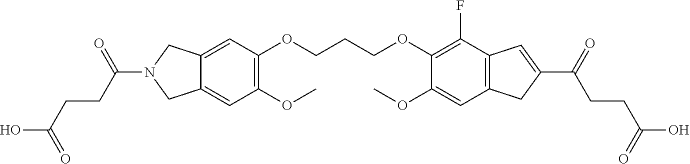 [SMILES: COc1cc2c(cc1OCCCOc1c(OC)cc3c(c1F)C=C(C(=O)CCC(=O)O)C3)CN(C(=O)CCC(=O)O)C2] | 586 |
| 126. | 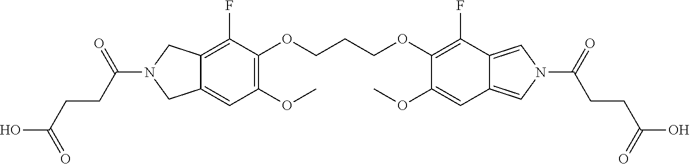 [SMILES: COc1cc2c(c(F)c1OCCCOc1c(OC)cc3cn(C(=O)CCC(=O)O)cc3c1F)CN(C(=O)CCC(=O)O)C2] | 605 |
| 127. | 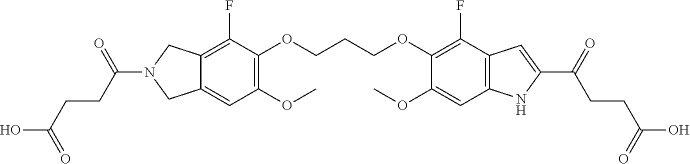 [SMILES: COc1cc2c(c(F)c1OCCCOc1c(OC)cc3[nH]c(C(=O)CCC(=O)O)cc3c1F)CN(C(=O)CCC(=O)O)C2] | 605 |
| 128. | 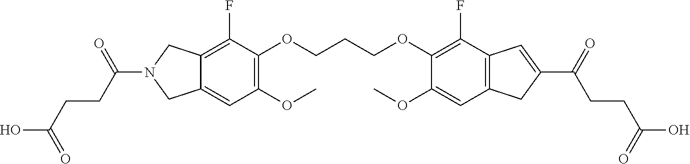 [SMILES: COc1cc2c(c(F)c1OCCCOc1c(OC)cc3c(c1F)CN(C(=O)CCC(=O)O)C3)C=C(C(=O)CCC(=O)O)C2] | 604 |

### TEXTING Example I: Human STING WT Binding Assay

Cisbio Bioassays' human STING WI binding assay (#64BDSTGPEG & 64BDSTGPEH, Cisbio) is for quantitative measurement of human STING WI ligand using HTRF® technology.

### 1. Adding Compounds

Negative control: Dispense 5 μL of diluent into each negative control well. Standard: Dispense 5 μL of each Human STING WI Standard 2′3′-cGAMP (Std 0-Std 7) into each standard well. Compound: Dispense 5 μL of compound into each compound well.

### 2. Adding Proteins

Negative control: Add 5 μL of detection buffer to all wells. Other wells: Add 5 μL of human STING WT protein 6His-tagged protein to all wells.

### 3. Adding Antibodies

Add 10 μL of premixed STING WT ligand d2 reagent and 6His T_{b} antibody working solution to all wells.

### 4. RT Incubation

Seal the plate and incubate 3 hours at RT or at Over Night if necessary.

### 5. Reading Plate

Remove the plate sealer and read on an HTRF® compatible reader (PerkinElmer, USA). Results were analyzed with a two-wavelength signal ratio: intensity (665 nm)/intensity (620 nm).

### 6. Curve Fitting

Calculate HTRF Ratio:

$$Ratio = \frac{Signal 665 nm}{Signal 620 nm} \times {10}^{4}$$

Fit the data in GraphPad to obtain IC₅₀ values using equation (2)
Equation (2): Y=Bottom+(Top-Bottom)/(1+10((LogIC₅₀—X)*Hill Slope))
Y is HTRF Ratio and X is compound concentration.
IC₅₀ value of binding assay for human STING WT:

| Example | hSTING WT binding IC₅₀/nM |
|---|---|
| I-1 | <0.1 |
| I-2 | 33.67 |
| I-3 | 1.63 |
| I-4 | 0.68 |
| I-5 | 3.28 |
| I-7 | >150 |
| I-8 | >150 |
| I-9 | 0.32 |
| I-10 | 150.10 |
| I-11 | >150 |
| I-12 | >150 |
| I-13 | 148.80 |
| I-14 | 143.50 |

Series 2—Compounds of Formula i, ii, iii, iv and v Intermediates

### INT A1: ethyl 4-(5-hydroxy-6-methoxy-benzothiophen-2-yl)-4-oxo-butanoate

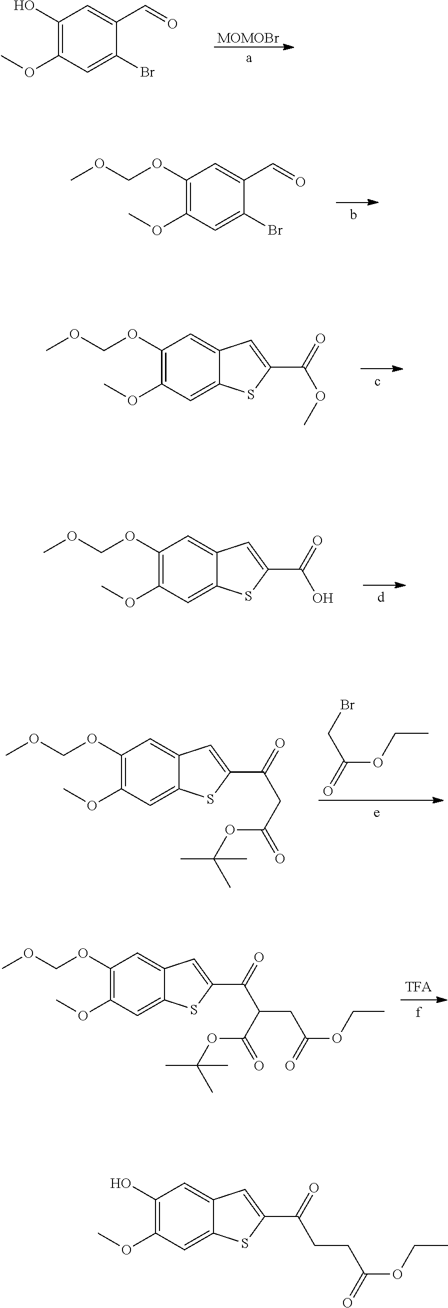

Step a: Bromomethyl methyl ether, (3.15 g, 25.2073 mmol) was added to a solution of DIEA (4.10 g, 46.5111 mmol) and 2-Bromoisovanillin (4.98 g, 21.5544 mmol) in Dichloromethane (200 mL) at 0° C. The reaction mixture was stirred at 20° C. for 2 h. The resulting solution was quenched by water (10 mL) and washed with water (100 mL) and brine (100 mL). The organics was dried over Na₂SO₄, filtered and evaporated under reduced pressure. There was 2-bromo-4-methoxy-5-(methoxymethoxy)benzaldehyde (5.83 g, 21.1926 mmol, 98.3216% yield) obtained as a white solid. ¹H NMR (400 MHz, DMSO-d₆) δ 10.06 (s, 1H), 7.51 (s, 1H), 7.39 (s, 1H), 5.25 (s, 2H), 3.93 (s, 3H), 3.38 (d, J=9.3 Hz, 3H).

Step b: Methyl thioglycolate (2.30 g, 21.6688 mmol) was added to a solution of 2-bromo-4-methoxy-5-(methoxymethoxy)benzaldehyde (5.83 g, 21.1926 mmol) and Cs₂CO₃ (13.99 g, 42.9380 mmol) in N,N-Dimethylformamide (100 mL) at 20° C. The reaction mixture was heated to 50° C. and stirred for 16 h. The reaction mixture was cooled to 20° C., and Methyl Iodide (1.81 g, 12.7520 mmol) was added to the reaction solution, and stirred for 2 h at 20° C. The reaction mixture was diluted with EA (500 mL), washed with water (2×100 mL) and brine (100 mL). The organics was evaporated under reduced pressure. The residue was purified on silica gel column EA/hept (0-50%). The pure fraction was concentrated and dried under vacuo. There was methyl 6-methoxy-5-(methoxymethoxy)benzothiophene-2-carboxylate (2.98 g, 10.5557 mmol, 49.8084% yield) obtained as a yellow solid. ¹H NMR (400 MHz, DMSO-d₆) δ 8.05 (s, 1H), 7.66 (s, 1H), 7.63 (s, 1H), 5.23 (s, 2H), 3.87 (d, J=9.2 Hz, 6H), 3.42 (s, 3H).

Step c: LiOH (0.37 g, 15.4499 mmol) was added to a solution of methyl 6-methoxy-5-(methoxymethoxy)benzothiophene-2-carboxylate (2.98 g, 10.5557 mmol) in Tetrahydrofuran (100 mL) and Water (25 mL) at 20° C. The reaction mixture was heated to 50° C. and stirred for 2 h. The reaction mixture was evaporated under reduced pressure and diluted with water and adjusted pH=3 with HCl (1 M). The precipitate was filtered. Filter cake was washed with water (20 mL). There was 6-methoxy-5-(methoxymethoxy) benzothiophene-2-carboxylic acid (2.41 g, 8.9830 mmol, 85.1008% yield) obtained as a white solid. ¹H NMR (400 MHz, DMSO-d₆) δ 13.22 (s, 1H), 7.93 (s, 1H), 7.62 (d, J=10.5 Hz, 2H), 5.24 (d, J=14.6 Hz, 2H), 3.87 (s, 3H), 3.52-3.35 (m, 3H).

Step d: To a stirred solution of 6-methoxy-5-(methoxymethoxy)benzothiophene-2-carboxylic acid (2.41 g, 8.9830 mmol) in N,N-Dimethylformamide (20 mL) was added 1,1′-Carbonyldiimidazole (2.84 g, 17.5148 mmol) at 20° C.

The resulting mixture was stirred for 1 h at 20° C. under nitrogen atmosphere. To the mixture above was added 3-tert-Butoxy-3-oxopropanoic acid and Magnesium chloride (1.31 g, 13.7589 mmol). The reaction mixture was stirred at 20° C. for 16 h. The reaction mixture was concentrated and diluted with EA (500 mL), washed with NaHCO₃ aq. (2×300 mL) and brine (200 mL). The organics was dried over Na₂SO₄, filtered and evaporated under reduced pressure. The residue was purified on silica gel column EA/PE (0-80%). The pure fraction was concentrated and dried under vacuo. There was tert-butyl 3-[6-methoxy-5-(methoxymethoxy) benzothiophen-2-yl]-3-oxo-propanoate (2.63 g, 7.1774 mmol, 79.9000% yield) obtained as a yellow oil. ¹H NMR (400 MHz, DMSO-d₆) δ 8.20 (s, 1H), 7.67 (s, 1H), 7.62 (s, 1H), 5.24 (s, 2H), 4.03 (d, J=9.8 Hz, 2H), 3.89 (s, 3H), 3.42 (s, 3H), 1.41 (s, 9H).

Step e: To a stirred solution of tert-butyl 3-[6-methoxy-5-(methoxymethoxy)benzothiophen-2-yl]-3-oxo-propanoate (2.62 g, 7.1501 mmol) in N,N-Dimethylformamide (20 mL) was added Potassium carbonate (2.0621 g, 14.9207 mmol) and Ethyl bromoacetate (1.3648 g, 8.1724 mmol) at 20° C. . The resulting mixture was stirred for 3 h at room temperature under nitrogen atmosphere. The reaction mixture was concentrated and diluted with EA (200 mL), washed water with (100 mL) and brine (50 mL). The organics was dried over Na₂SO₄, filtered and evaporated under reduced pressure. The residue was purified on silica gel column EA/PE (0-80%). The pure fraction was concentrated and dried under vacuo. There was 01-tert-butyl 04-ethyl 2-[6-methoxy-5-(methoxymethoxy) benzothiophene-2-carbonyl]butanedioate (2.82 g, 6.2318 mmol, 87.1569% yield) obtained as a yellow oil. ¹H NMR (400 MHz, DMSO-d₆) δ 8.35 (s, 1H), 7.67 (s, 2H), 5.31-5.20 (m, 2H), 4.79 (t, J=7.3 Hz, 1H), 4.15-3.96 (m, 2H), 3.89 (d, J=6.7 Hz, 3H), 3.42 (d, J=4.2 Hz, 3H), 3.32 (s, 3H), 2.98-2.81 (m, 2H), 1.31 (s, 9H).

Step f: TFA (5 mL) was added to 01-tert-butyl 04-ethyl 2-[6-methoxy-5-(methoxymethoxy)benzothiophene-2-carbonyl]butanedioate (2.82 g, 6.2318 mmol) in Toluene (25 mL) at 20° C. The reaction mixture was heated to 50° C. and stirred for 30 min. The reaction mixture was evaporated under reduced pressure. The reaction mixture was concentrated and diluted with EA (200 mL), washed with water (100 mL) and brine (50 mL). The organics was dried over Na₂SO₄, filtered and evaporated under reduced pressure. The residue was purified on silica gel column EA/Heptane (0-100%). The pure fraction was concentrated and dried under vacuo to afford crude product. The residue was purified on silica gel column MeCN/water (0-100%). The pure fraction was concentrated and dried under vacuo. There was ethyl 4-(5-hydroxy-6-methoxy-benzothiophen-2-yl)-4-oxo-butanoate (0.79 g, 2.5620 mmol, 41.1122% yield) obtained as a white solid. ¹H NMR (400 MHz, DMSO-d₆) δ 9.39 (s, 1H), 8.17 (s, 1H), 7.54 (s, 1H), 7.32 (s, 1H), 4.06 (q, J=7.1 Hz, 2H), 3.87 (s, 3H), 3.31-3.26 (m, 2H), 2.65 (t, J=6.3 Hz, 2H), 1.27-1.05 (m, 3H).

### INT A2: methyl 4-(5-chloro-6-methoxythieno[3,2-b]pyridin-2-yl)-4-oxobutanoate

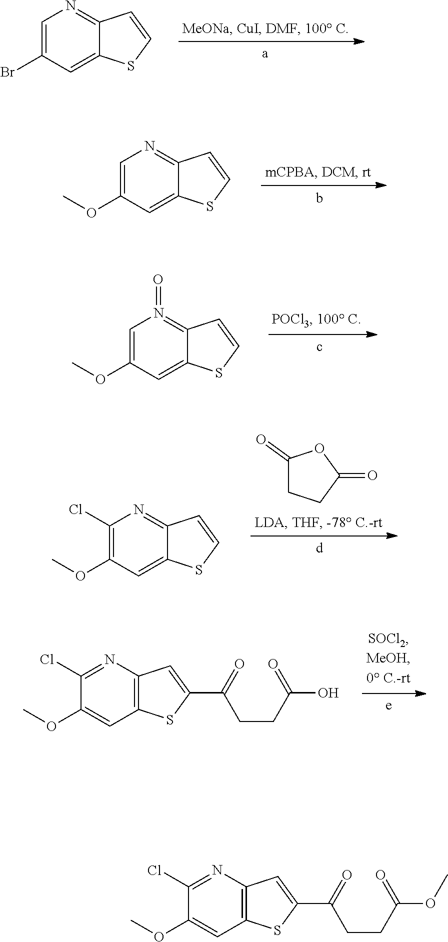

Step a: To a solution of 6-bromothieno[3,2-b]pyridine (30.0 g, 140.1 mmol) in DMF (300 mL) was added a solution of 25% MeONa in MeOH (605.6 g, 2802.6 mmol) and CuI (26.7 g, 140.1 mmol). The reaction mixture was heated to 100° C. and stirred for 2 h. Then the reaction mixture was cooled to room temperature, poured into ice water (900 mL) and filtered. The filtrate was diluted with NH₃·H₂O (300 mL), extracted with EtOAc (1.0 μL×2) and washed with brine (1.0 L). The organic layer was dried over Na₂SO₄, filtered and the filtrate was concentrated to afford 6-methoxythieno[3,2-b]pyridine (19.0 g, 82.3%) as a yellow solid which was used for next step without further purification. ¹H NMR (300 MHz, CDCl₃): δ 8.43 (d, J=2.7 Hz, 1H), 7.62 (d, J=2.4 Hz, 1H), 7.53-7.51 (m, 1H), 7.47-7.45 (m, 1H), 3.91 (s, 3H). LCMS: (ESI, m/z): [M+H]⁺=166.2.

Step b: To a solution of 6-methoxythieno[3,2-b]pyridine (19.0 g, 115.0 mmol) in DCM (200 mL) was added m-CPBA (19.8 g, 115.0 mmol). The reaction mixture was stirred at room temperature for 16 h. Then the reaction mixture was diluted with DCM/MeOH=10:1 (300 mL) and washed with saturated sodium bicarbonate solution (300 mL×2) and brine (300 mL). The organic layer was dried over Na₂SO₄, filtered and the filtrate was concentrated to afford 6-methoxythieno[3,2-b]pyridine 4-oxide (13.0 g, 57.7%) as a yellow solid which was used for next step without further purification. ¹H NMR (300 MHz, CDCl₃): δ 8.14 (d, J=2.1 Hz, 1H), 7.74 (dd, J=5.7 Hz, 0.6 Hz, 1H), 7.48-7.46 (m, 1H), 7.31-7.30 (m, 1H), 3.91 (s, 3H). LCMS: (ESI, m/z): [M+H]⁺=182.0.

Step c: Add 6-methoxythieno[3,2-b]pyridine 4-oxide (15.5 g, 85.5 mmol) portion wise to POCl₃ (200 mL) at room temperature. The reaction mixture was heated to 100° C. and stirred for 16 h. Then the reaction mixture was cooled to room temperature and concentrated. The residue was diluted with water (200 mL), adjusted to pH=8 with saturated sodium bicarbonate solution and extracted with DCM (300 mL×2). The organic layers were washed with brine (200 mL), dried over Na₂SO₄, filtered and the filtrate was concentrated to give the residue which was purified by column chromatography on silica gel (eluted with petroleum ether/EtOAc=10:1) to afford 5-chloro-6-methoxythieno[3,2-b]pyridine (9.0 g, 52.6%) as a white solid. ¹H NMR (300 MHz, CDCl₃): δ 7.65 (s, 1H), 7.58-7.56 (m, 1H), 7.42 (dd, J=5.7 Hz, 0.6 Hz, 1H), 3.99 (s, 3H). LCMS: (ESI, m/z): [M+H]⁺=200.0.

Step d: To a solution of 5-chloro-6-methoxythieno[3,2-b]pyridine (8.0 g, 40.1 mmol) in THF (80 mL) was added LDA (2.0 M, 80 mL) dropwise at −78° C. under nitrogen. After 15 min, a solution of dihydrofuran-2,5-dione (22.0 g, 220.4 mmol) in THF (240 mL) was added to the mixture dropwise at −40° C. under nitrogen. Then the reaction mixture was warmed to room temperature and stirred for 2 h. After that the reaction was quenched with 2N HCl (100 mL) and extracted with EtOAc (300 mL×2). The organic layers were washed with brine (200 mL), dried over Na₂SO₄, filtered and the filtrate was concentrated. The residue was triturated with petroleum ether/EtOAc=1:1 and filtered to afford 4-(5-chloro-6-methoxythieno[3,2-b]pyridin-2-yl)-4-oxobutanoic acid (6.0 g, crude) as a white solid which was used for next step without further purification. LCMS: (ESI, m/z): [M−H]—=298.8.

Step e: To a solution of 4-(5-chloro-6-methoxythieno[3,2-b]pyridin-2-yl)-4-oxobutanoic acid (6.0 g crude, 20.0 mmol) in MeOH (120 mL) was added SOCl₂ (12.0 g, 100.1 mmol) dropwise at 0° C. Then the reaction mixture was warmed to room temperature and stirred for 2 h. After that the reaction was concentrated, diluted with water (100 mL), adjusted to pH=8 with saturated sodium bicarbonate solution and extracted with DCM/MeOH (200 mL×2). The organic layers were washed with brine (100 mL), dried over Na₂SO₄, filtered. The filtrate was concentrated, white solid precipitated and filtered to afford methyl 4-(5-chloro-6-methoxythieno[3,2-b]pyridin-2-yl)-4-oxobutanoate (1.1 g, 8.5% in total of d and e two steps) as a white solid ¹H NMR (400 MHz, CDCl₃): δ 8.01 (s, 1H), 7.59 (s, 1H), 4.02 (s, 3H), 3.71 (s, 3H), 3.35 (t, J=6.8 Hz, 2H), 2.80 (t, J=6.8 Hz, 2H), LCMS: (ESI, m/z): [M+H]⁺=569.20.

### INT A3: methyl trans-2-(5-bromo-6-methoxybenzo[b]thiophene-2-carbonyl)cyclopropane-1-carboxylate

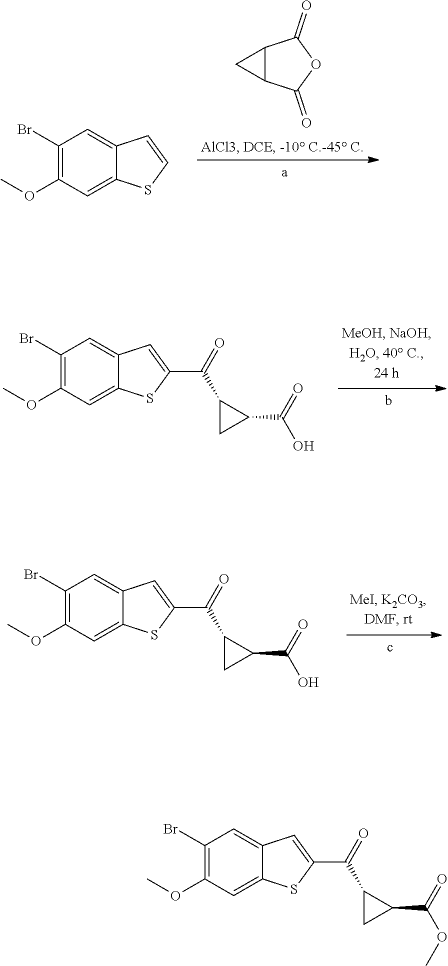 [SMILES: O=C1OC(=O)C2CC12 ; COc1cc2sccc2cc1Br ; COc1cc2sc(C(=O)[C@H]3C[C@H]3C(=O)O)cc2cc1Br ; COc1cc2sc(C(=O)[C@H]3C[C@@H]3C(=O)O)cc2cc1Br ; COC(=O)[C@H]1C[C@@H]1C(=O)c1cc2cc(Br)c(OC)cc2s1]

Step a: To a solution of 3-oxabicyclo [3.1.0]hexane-2,4-dione (6.3 g, 56.1 mmol) and AlCl₃ (12.3 g, 92.6 mmol) in DCE (150 mL) was added a solution of 5-bromo-6-methoxybenzo[b]thiophene (15.0 g, 61.7 mmol) in DCE (50 mL) at −10° C. After that the reaction mixture was heated to 45° C. and stirred for 16 h. Then the reaction mixture was poured into ice-water (200 mL) and extracted with DCM/MeOH=10:1 (100 mL×2). The organic layer washed with brine (100 mL), dried over Na₂SO₄, filtered and the filtrate was concentrated to dryness. The residue was triturated with Petroleum ether/EtOAc=1:1 (50 mL) and filtered to afford cis-2-(5-bromo-6-methoxybenzo[b]thiophene-2-carbonyl)cyclopropane-1-carboxylic acid (3.0 g, yield 15.1%) as a yellow solid. ¹H NMR (400 MHz, DMSO-d₆): δ 8.32 (s, 1H), 8.26 (s, 1H), 7.80 (s, 1H), 4.05 (s, 3H), 3.01-3.09 (m, 1H), 2.25-2.31 (m, 1H), 1.65-1.68 (m, 1H), 1.58-1.52 (m, 1H).

Step b: To a solution of cis -2-(5-bromo-6-methoxybenzo[b]thiophene-2-carbonyl)cyclopropane-1-carboxylic acid (3.0 g, 8.4 mmol) in MeOH (30 mL) was added 50% aq. NaOH (45 mL) at room temperature. Then the reaction mixture was heated to 40° C. and stirred for 24 h. After the reaction completed analysis by HPLC, the solvent was removed by concentrated. The residue was diluted with water (30 mL) and was adjusted pH-2 with 6 N HCl. The white solid was collected by filtration. The solid was dryness under vacuum at 50° C. to afford trans-2-(5-bromo-6-methoxybenzo[b]thiophene-2-carbonyl)cyclopropane-1-carboxylic acid (2.5 g, yield 83.3%) as a yellow solid. ¹H NMR (400 MHz, DMSO-d₆): δ 8.52 (s, 1H), 8.27 (s, 1H), 7.80 (s, 1H), 3.96 (s, 3H), 3.27-3.23 (m, 1H), 2.16-2.11 (m, 1H), 1.54-1.49 (m, 2H).

Step c: To a solution of trans-2-(5-bromo-6-methoxybenzo[b]thiophene-2-carbonyl)cyclopropane-1-carboxylic acid (2.5 g, 7.0 mmol) in DMF (25 mL) was added K₂CO₃ (2.4 g, 17.5 mmol) and Mel (2.0 g, 14.1 mmol) at room temperature. The reaction mixture was stirred for 16 h. Then the mixture was poured into water (50 mL) and extracted with EtOAc (50 mL×2). The organic layer was washed with water (50 mL×2) and brine (50 mL×2). The organic layer was dried with anhydrous Na₂SO₄, filtered and the filtrate was concentrated to dryness. The residue was triturated with Petroleum ether/EtOAc=5:1 (50 mL) and filtered to afford methyl trans-2-(5-bromo-6-methoxybenzo[b]thiophene-2-carbonyl)cyclopropane-1-carboxylate (2 g, yield 77.5%) as a yellow solid. LCMS: (ESI, m/z): [M+H]⁺=368.8, 370.8. ¹H NMR (400 MHz, CDCl₃): δ 8.07 (s, 1H), 7.96 (s, 1H), 7.30 (s, 1H), 3.99 (s, 3H), 3.75 (s, 3H), 3.15-3.11 (m, 1H), 2.45-2.40 (m, 1H), 1.70-1.64 (m, 2H).

### INT A4: ethyl 4-(4-chloro-5-hydroxy-6-methoxybenzo[b]thiophen-2-yl)-4-oxobutanoate

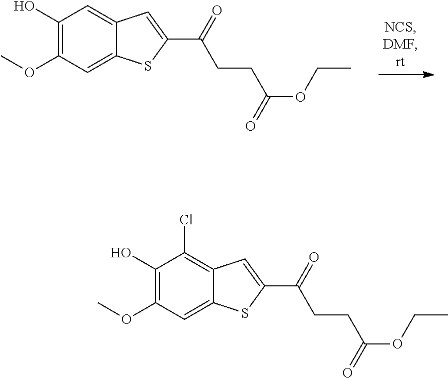

To a solution of ethyl 4-(5-hydroxy-6-methoxybenzo[b]thiophen-2-yl)-4-oxobutanoate (300 mg, 0.97 mmol) in DMF (6 mL) was added NCS (156 mg, 1.14 mmol) portion wise at room temperature. After that the reaction mixture was stirred for 5 h. Then the reaction mixture was poured into ice-water (18 mL). The mixture was filtered and the filter cake was dissolved with EtOAc (20 mL). The organic layer was washed with sat. aq. NaHCO₃ (15 mL) and brine (10 mL), dried over Na₂SO₄ and concentrated to afford a crude product which was purified by HPLC to give ethyl 4-(4-chloro-5-hydroxy-6-methoxybenzo[b]thiophen-2-yl)-4-oxobutanoate (240 mg, yield 72%) as a white solid. ¹H NMR (300 MHz, CDCl₃): δ 8.02 (s, 1H), 7.17 (s, 1H), 6.04 (brs, 1H), 4.20-4.12 (m, 2H), 4.02 (s, 3H), 3.36 (t, J=8.8 Hz, 2H), 2.79 (t, J=8.8 Hz, 2H), 1.30-1.20 (m, 3H). LCMS: (ESI, m/z): [M+H]⁺=342.9.

### INT A5: ethyl 4-(5-methoxyisoindolin-2-yl)-4-oxo-butanoate

### INT A6: ethyl 4-(5-bromo-6-methoxy-isoindolin-2-yl)-4-oxo-butanoate

### INT A7: ethyl 4-(5-hydroxy-6-methoxyisoindolin-2-yl)-4-oxobutanoate

### AND INT A8: ethyl 4-(5-(3-bromopropoxy)-6-methoxyisoindolin-2-yl)-4-oxobutanoate

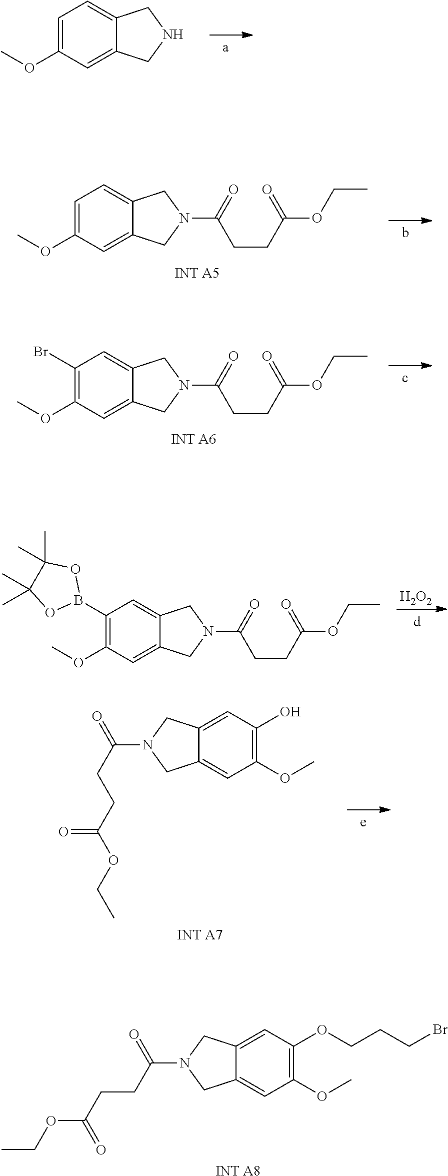

Step a: Succinic anhydride (4.40 g, 43.9681 mmol) was added to a solution of 5-Methoxyisoindoline hydrochloride (4.89 g, 26.3399 mmol) and Triethylamine (4.73 g, 46.7440 mmol) in Ethanol (200 mL) at 20° C. The reaction mixture was stirred for 1 h at 20° C. SOC₂ (20 mL) was added to the solution above at 0° C. The reaction mixture was stirred for 3 h at 20° C. The reaction mixture was evaporated under reduced pressure. The reaction mixture was diluted with EA (200 mL), washed with water (100 mL) and brine (50 mL). The organics was dried over Na₂SO₄, filtered and evaporated under reduced pressure. There was ethyl 4-(5-methoxyisoindolin-2-yl)-4-oxo-butanoate (17.5 g, 31.5526 mmol, 119.7901% yield) obtained as a yellow solid.

¹H NMR (400 MHz, DMSO-d₆) δ 7.28-7.21 (m, 1H), 6.94 (s, 0.5H), 6.92 (s, 0.5H), 6.88 (s, 0.5H), 6.86 (s, 0.5H), 4.81 (s, 1H), 4.76 (s, 1H), 4.58 (s, 1H), 4.54 (s, 1H), 4.02-3.95 (m, 2H), 3.75 (s, 3H), 2.65-2.58 (m, 2H), 2.58-2.54 (m, 2H), 1.17 (t, J=3.5 Hz, 3H).

Step b: NBS (10.51 g, 59.0503 mmol) was added to ethyl 4-(5-methoxyisoindolin-2-yl)-4-oxo-butanoate in Tetrahydrofuran (100 mL) and Acetonitrile (100 mL) at 20° C. The reaction mixture was stirred overnight at 20° C.

The reaction mixture was evaporated under reduced pressure. The reaction mixture was concentrated and diluted with DCM (500 mL), washed with NaHCO₃ aq. (2×300 mL) and brine (200 mL). The organics was dried over Na₂SO₄, filtered and evaporated under reduced pressure. The residue was purified on silica gel column MeOH/DCM (0-10%). The pure fraction was concentrated and dried under vacuo. There was ethyl 4-(5-bromo-6-methoxy-isoindolin-2-yl)-4-oxo-butanoate (7.26 g, 20.3812 mmol, 64.5943% yield) obtained as a yellow solid.

¹H NMR (400 MHz, DMSO-d₆) δ 7.58 (s, 0.5H), 7.57 (s, 0.5H), 7.15 (s, 0.5H), 7.12 (s, 0.5H), 4.80 (s, 1H), 4.77 (s, 1H), 4.57 (s, 1H), 4.54 (s, 1H), 4.09-4.01 (m, 2H), 3.84 (s, 3H), 2.65-2.59 (m, 2H), 2.56-2.53 (m, 2H), 1.18 (t, J=7.1 Hz, 3H).

Step c: Potassium Acetate (3.74 g, 38.1080 mmol) was added to Pd(dppf)Cl₂ (0.72 g, 984.0050 mol), ethyl 4-(5-bromo-6-methoxy-isoindolin-2-yl)-4-oxo-butanoate (3.64 g, 10.2187 mmol) and 4,4,4′,4′,5,5,5′,5′-Octamethyl-2,2′-bi(1,3,2-dioxaborolane) (3.84 g, 15.1218 mmol) in 1,4-Dioxane (100 mL) at 20° C. The reaction mixture was stirred at 100° C. for 16 h. The reaction mixture was evaporated under reduced pressure. The reaction mixture was concentrated and diluted with DCM (500 mL), washed with NaHCO₃ aq. (2×300 mL) and brine (200 mL). The organics was dried over Na₂SO₄, filtered and evaporated under reduced pressure. The residue was purified on silica gel column EA/Hept (0-100%). The pure fraction was concentrated and dried under vacuo. There was ethyl 4-(5-methoxy-6-(4,4,5,5-tetramethyl-1,3,2-dioxaborolan-2-yl)isoindolin-2-yl)-4-oxobutanoate (2.45 g, 6.0752 mmol, 59.7809% yield) obtained as a yellow solid.

¹H NMR (400 MHz, DMSO-d₆) δ 7.49 (s, 0.5H), 7.48 (s, 0.5H), 6.98 (s, 0.5H), 6.95 (s, 0.5H), 4.83 (s, 1H), 4.74 (s, 1H), 4.60 (s, 1H), 4.53 (s, 1H), 4.09-4.01 (m, 2H), 3.74 (t, J=6.2 Hz, 3H), 2.65-2.58 (m, 2H), 2.58-2.52 (m, 2H), 1.27 (s, 12H), 1.21-1.13 (m, 3H).

Step d: Sodium perborate tetrahydrate (1 g, 6.4994 mmol) was added to ethyl 4-(5-methoxy-6-(4,4,5,5-tetramethyl-1,3,2-dioxaborolan-2-yl)isoindolin-2-yl)-4-oxobutanoate (2.45 g, 6.0752 mmol) in Tetrahydrofuran (20 mL) and Water (20 mL) at 20° C. The reaction mixture was stirred for 1 h at 20° C. The reaction mixture was evaporated under reduced pressure. The reaction mixture was concentrated and diluted with DCM (500 mL), washed with NaHCO₃ aq. (2×300 mL) and brine (200 mL). The organics was dried over Na₂SO₄, filtered and evaporated under reduced pressure. The residue was purified on silica gel column MeOH/DCM (0-10%). The pure fraction was concentrated and dried under vacuo. There was ethyl 4-(5-hydroxy-6-methoxyisoindolin-2-yl)-4-oxobutanoate (1.37 g, 4.6708 mmol, 76.8818% yield) obtained as a yellow solid.

¹H NMR (400 MHz, DMSO-d₆) δ 8.97 (s, 0.5H), 8.96 (s, 0.5H), 6.91 (s, 0.5H), 6.89 (s, 0.5H), 6.73 (s, 0.5H), 6.73 (s, 0.5H), 4.71 (s, 1H), 4.69 (s, 1H), 4.50 (s, 1H), 4.47 (s, 1H), 4.08-4.00 (m, 2H), 3.75 (s, 3H), 2.64-2.58 (m, 2H), 2.56-2.53 (m, 2H), 1.18 (t, J=7.1 Hz, 3H).

Step e: To a solution of ethyl 4-(5-hydroxy-6-methoxy-isoindolin-2-yl)-4-oxo-butanoate (0.72 g, 2.4547 mmol) in N,N-Dimethylformamide (10 mL) was added 1,3-Dibromopropane (0.73 g, 3.6159 mmol) and potassium carbonate (0.86 g, 6.2226 mmol)₂. This mixture was stirred for 16 hours at 50° C. The reaction mixture was diluted with Ethyl acetate (100 mL), and washed sequentially with water (2×100 mL) and saturated brine (1×100 mL). The organic layer was dried over Na₂SO₄, filtered and evaporated to afford crude product. The crude product was purified by flash silica chromatography, elution gradient 0 to 100% Ethyl acetate in heptane. There was ethyl 4-(5-(3-bromopropoxy)-6-methoxyisoindolin-2-yl)-4-oxobutanoate (0.43 g, 1.0379 mmol, 42.2829% yield) obtained as a white solid.

¹H NMR (400 MHz, DMSO-d₆) δ 7.01-6.93 (m, 2H), 4.75 (s, 2H), 4.53 (s, 2H), 4.10-4.00 (m, 4H), 3.76 (s, 3H), 3.71-3.62 (m, 2H), 2.65-2.58 (m, 2H), 2.58-2.53 (m, 2H), 2.28-2.18 (m, 2H), 1.18 (t, J=7.1 Hz, 3H).

### INT A9: tert-butyl 3-(5-bromo-4-fluoro-6-methoxy-benzothiophen-2-yl)-3-oxo-propanoate

### Step a: 3-bromo-2-fluoro-4-methoxy-benzaldehyde

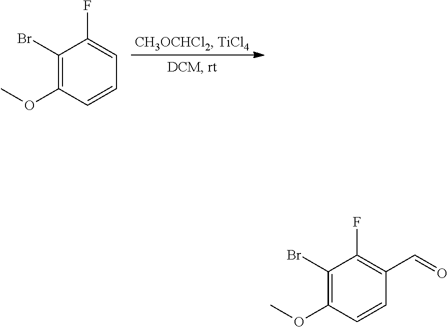

Titanium tetrachloride (38.06 g, 200.6548 mmol) was added dropwise to a solution of 2-Bromo-3-fluoroanisole (10.17 g, 49.6039 mmol) in Dichloromethane (250 mL) at 0° C. for 1 h. The reaction mixture was stirred for 3 h at 20° C. The reaction mixture was quenched with adding of ice-water (200 mL) at 0° C. The reaction mixture was extracted with DCM (2×100 mL), washed with NaHCO₃ (1×200 mL) and brine (100 mL). The organics dried over Na₂SO₄, filtered and evaporated under reduced pressure. The residue was dried under vacuo at 60° C. There was 3-bromo-2-fluoro-4-methoxy-benzaldehyde (11.42 g, 49.0057 mmol, 98.7940% yield) obtained as a white solid. LCMS: (ESI, m/z): [M+H]⁺=232.954. ¹H NMR (400 MHz, CDCl₃-d) 6 10.24 (s, 1H), 7.87 (t, J=8.3 Hz, 1H), 6.84 (d, J=8.8 Hz, 1H), 4.05-3.99 (m, 3H).

### Step b: (5E)-5-[(3-bromo-2-fluoro-4-methoxy-phenyl)methylene]-2-thioxo-thiazolidin-4-one

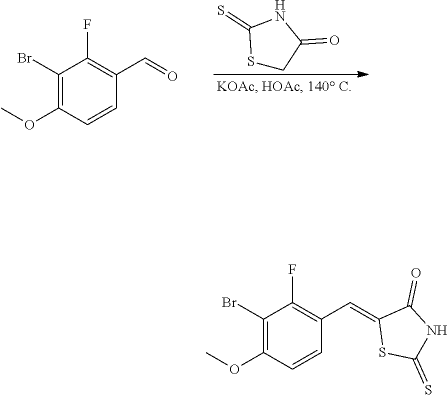 [SMILES: COc1ccc(C=O)c(F)c1Br ; O=C1CSC(=S)N1 ; COc1ccc(/C=C2\SC(=S)NC2=O)c(F)c1Br]

Potassium Acetate (12.67 g, 129.0984 mmol) was added to a mixture of 3-bromo-2-fluoro-4-methoxy-benzaldehyde (10.01 g, 42.9551 mmol) and Rhodanine (5.72 g, 42.9456 mmol) in Acetic acid (100 mL) at 20° C. The reaction mixture was heated to 140° C. and stirred overnight. The reaction mixture was added to water (1000 mL) and stirred for 10 min. The precipitate was collected by filtration, washed with water (200 mL). The filtrate cake was dried under vacuo at 60° C. There was (5E)-5-[(3-bromo-2-fluoro-4-methoxy-phenyl)methylene]-2-thioxo-thiazolidin-4-one (9.21 g, 26.4495 mmol, 61.5748% yield) obtained as a yellow solid. LCMS: (ESI, m/z): [M+H]⁺=347.909. H NMR (400 MHz, DMSO-d₆) δ 13.87 (s, 1H), 7.68-7.32 (m, 2H), 7.16 (d, J=8.7 Hz, 1H), 3.97 (s, 3H).

### Step c: (Z)-3-(3-bromo-2-fluoro-4-methoxy-phenyl)-2-sulfanyl-prop-2-enoic acid

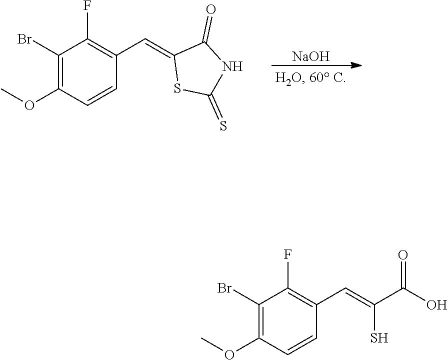 [SMILES: COc1ccc(/C=C2\SC(=S)NC2=O)c(F)c1Br ; "NaOH" ; COc1ccc(/C=C(\S)C(=O)O)c(F)c1Br]

To a stirred mixture of (5E)-5-[(3-bromo-2-fluoro-4-methoxy-phenyl)methylene]-2-thioxo-thiazolidin-4-one (8.03 g, 23.0607 mmol) in Water (200 mL) was added NaOH (9.15 g, 228.7666 mmol) in water (80 mL) at 20° C. The reaction mixture was stirred at 60° C. for 1 h. After the reaction completed, adjusted to pH=3 with HCl. The precipitate was collected by filtration, washed with water (1×100 mL). The filtrate cake was dried under vacuo at 60° C. There was (Z)-3-(3-bromo-2-fluoro-4-methoxy-phenyl)-2-sulfanyl-prop-2-enoic acid (7.37 g, 23.9959 mmol, 104.0552% yield) obtained as a yellow solid. LCMS: (ESI, m/z): [M+H]⁺=306.936.

### Step d: 5-bromo-4-fluoro-6-methoxy-benzothiophene-2-carboxylic acid

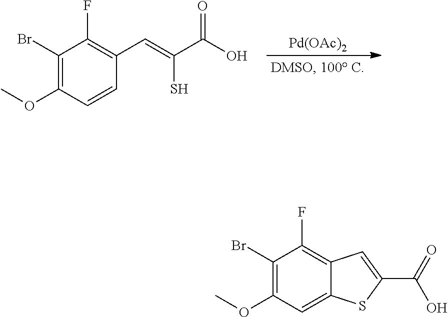 [SMILES: COc1ccc(/C=C(\S)C(=O)O)c(F)c1Br ; COc1cc2sc(C(=O)O)cc2c(F)c1Br ; "Pd(OAc)2"]

Palladium (II) acetate (4.0934 g, 18.2330 mmol) was added to a solution of (Z)-3-(3-bromo-2-fluoro-4-methoxy-phenyl)-2-sulfanyl-prop-2-enoic acid (7 g, 22.7912 mmol) in Methyl sulfoxide (50 mL) at 20° C. under N₂ atmosphere. The reaction mixture was heated to 100° C. and stirred for 1 h. The reaction mixture was purified on C-18 column MeCN/water (0-100%). The pure fraction was concentrated and dried under vacuo. There was 5-bromo-4-fluoro-6-methoxy-benzothiophene-2-carboxylic acid (3.9 g, 12.7819 mmol, 56.0824% yield) obtained as a yellow solid. LCMS: (ESI, m/z): [M+H]⁺=304.921. ¹H NMR (400 MHz, DMSO-d₆) δ 7.96 (s, 1H), 7.70 (s, 1H), 3.97 (s, 3H).

### Step e: tert-butyl 3-(5-bromo-4-fluoro-6-methoxy-benzothiophen-2-yl)-3-oxo-propanoate

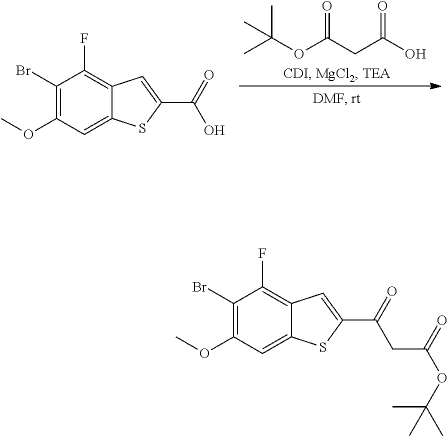

To a stirred solution of 5-bromo-4-fluoro-6-methoxy-benzothiophene-2-carboxylic acid (3.88 g, 12.7163 mmol) in DMF (50 mL) was added Carbonyl diimidazole (4.12 g, 25.4088 mmol) at 20° C. The resulting mixture was stirred for 1 h at 20° C. under nitrogen atmosphere. To the mixture above was added Magnesium chloride (1.81 g, 19.0104 mmol), 3-tert-Butoxy-3-oxopropanoic acid (3.13 g, 19.5421 mmol) and Triethylamine (3.91 g, 38.6404 mmol). The reaction mixture was stirred overnight at 20° C. The reaction mixture was concentrated and diluted with EA (500 mL), washed with NaHCO₃ aq (2×500 mL) and brine (100 mL). The organics dried over Na₂SO₄, filtered and evaporated under reduced pressure. The residue was purified by flash silica chromatography, elution gradient 0 to 100% Ethyl acetate in heptane. The pure fraction was concentrated and dried under vacuo. There was tert-butyl 3-(5-bromo-4-fluoro-6-methoxy-benzothiophen-2-yl)-3-oxo-propanoate (3.29 g, 8.1585 mmol, 64.1574% yield) obtained as a yellow solid. LCMS: (ESI, m/z): [M+H]⁺=402.994. ¹H NMR (400 MHz, DMSO-d₆) δ 8.38 (d, J=2.2 Hz, 1H), 7.74 (d, J=8.5 Hz, 1H), 4.16 (s, 2H), 3.97 (d, J=8.2 Hz, 3H), 1.41 (d, J=1.6 Hz, 1OH).

### INT A10: tert-butyl 4-fluoro-5-hydroxy-6-methoxy-isoindoline-2-carboxylate

### Step a: 4-fluoro-5-methoxy-isoindoline

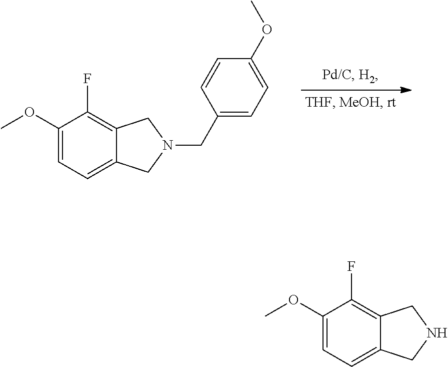 [SMILES: COc1ccc(CN2Cc3ccc(OC)c(F)c3C2)cc1 ; COc1ccc2c(c1F)CNC2]

10% Pd/C (7.68 g, contain 55% H₂O) was added to a solution of 4-fluoro-5-methoxy-2-[(4-methoxyphenyl)methyl]isoindoline (7.18 g, 24.9889 mmol) in THF (70 mL) and MeOH (70 mL) at 20° C. The reaction mixture was stirred overnight at 20° C. The reaction mixture was filtered and the filter cake was washed with MeOH (70 mL). The filtrate was evaporated under reduced pressure. There was 4-fluoro-5-methoxy-isoindoline (4.88 g, 29.1901 mmol, 100% yield) obtained as a brown oil, which was used directly in the next step.

LCMS: (ESI, m/z): [M+H]⁺=168.200.

### Step b: 4-fluoroisoindolin-5-ol hydrobromate

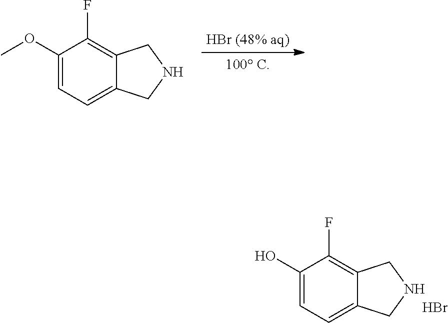

4-fluoro-5-methoxy-isoindoline (4.5 g, 26.9171 mmol) was dissolved in Hydrobromic acid (120 mL, 48% aq) at 20° C. The reaction mixture was heated to 100° C. and stirred for 6 h. The resulting reaction mixture was evaporated under reduced pressure. The residue was treated with n-hexane/EA (1:2). The solid was collected by filtration, and dried under reduced pressure. There was 4-fluoroisoindolin-5-ol hydrobromate (4.70 g, 20.0799 mmol, 74.60% yield) obtained as a brown solid. LCMS: (ESI, m/z): [M+H]⁺=154.200. ¹H NMR (400 MHz, DMSO-d₆) δ 10.09 (s, 1H), 9.58 (s, 2H), 7.10-6.93 (m, 2H), 4.57 (s, 2H), 4.44 (s, 2H).

### Step c: tert-butyl 4-fluoro-5-hydroxy-isoindoline-2-carboxylate

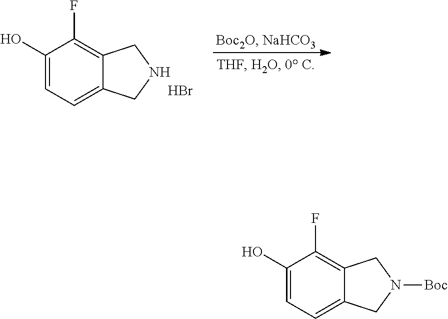 [SMILES: Oc1ccc2c(c1F)CNC2 ; Br ; CC(C)(C)OC(=O)N1Cc2ccc(O)c(F)c2C1]

NaHCO₃ (3.43 g, 40.8301 mmol) was added to a solution of 4-fluoroisoindolin-5-olhydrobromate (4.70 g, 20.0799 mmol) in Water (80 mL) at 0° C. After the reaction mixure was stirred for 20 min, THF (60 mL) was added to the reaction mixture, and Di-tert-butyl dicarbonate (4.45 g, 20.3898 mmol) in THF (20 mL) was added dropwise to the mixure at 0° C. After stirring for 20 min, the reaction mixure was warmed to 20° C. and stirred for 2 h. The resulting reaction mixture was diluted with EA (150 mL) and washed with brine (150 mL). The organic layer was dried over Na₂SO₄, filtered and evaporated under reduced pressure. The residue was purified by flash chromatography, eluting with ethyl acetate in n-hexane (0-40%). The pure fraction was concentrated and dried under reduced pressure. There was tert-butyl 4-fluoro-5-hydroxy-isoindoline-2-carboxylate (3.14 g, 12.3979 mmol, 60.78% yield) obtained as a reddish brown solid. LCMS: (ESI, m/z): [M−H]⁻=252.000. ¹H NMR (400 MHz, CDCl₃-d) 6 7.00-6.93 (m, 1H), 6.93-6.83 (m, 1H), 4.72 (s, 2H), 4.64 (s, 2H), 1.54 (s, 9H).

### Step d: tert-butyl 6-bromo-4-fluoro-5-hydroxy-isoindoline-2-carboxylate

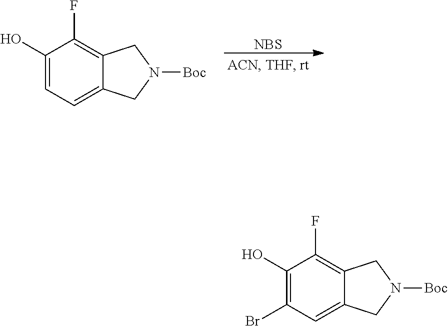 [SMILES: CC(C)(C)OC(=O)N1Cc2ccc(O)c(F)c2C1 ; "NBS" ; CC(C)(C)OC(=O)N1Cc2cc(Br)c(O)c(F)c2C1]

NBS (2.20 g, 12.3607 mmol) was added to a solution of tert-butyl 4-fluoro-5-hydroxy-isoindoline-2-carboxylate (3.10 g, 12.2400 mmol) in ACN (40 mL) and THF (20 mL) at 0° C. The reaction mixture was stirred for 2.5 h at 20° C. The reaction mixture was concentrated under reduced pressure. The residue was dissloved with EA/MeOH (200 mL/10 mL), and washed with H₂O (200 mL). The organic layer was dried over Na₂SO₄, filtered and evaporated in vacuo. The residue was purified by flash chromatography, eluting with MeOH in DCM (0-5%). The pure fraction was concentrated and dried under reduced pressure. There was tert-butyl 6-bromo-4-fluoro-5-hydroxy-isoindoline-2-carboxylate (2.70 g, 8.1285 mmol, 66.34% yield) obtained as a white solid. LCMS: (ESI, m/z): [M−H]⁻=331.950. ¹H NMR (400 MHz, DMSO-d₆) δ 10.36 (s, 1H), 7.33 (s, 0.5H), 7.32 (s, 0.5H), 4.58 (s, 1H), 4.55 (s, 1H), 4.52 (s, 1H), 4.50 (s, 1H), 1.45 (s, 9H).

### Step e: tert-butyl 5-benzyloxy-6-bromo-4-fluoro-isoindoline-2-carboxylate

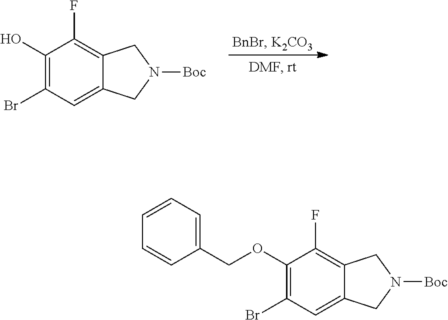 [SMILES: CC(C)(C)OC(=O)N1Cc2cc(Br)c(O)c(F)c2C1 ; CC(C)(C)OC(=O)N1Cc2cc(Br)c(OCc3ccccc3)c(F)c2C1]

K₂CO₃ (2.20 g, 15.9183 mmol) was added to a solution of tert-butyl 6-bromo-4-fluor6--hdroxy-isoindoline-2-carboxylate (2.70 g, 8.1285 mmol) in DMF (50 mL) at 0° C. After the reaction mixture was stirred for 0.5 h at 0° C., BnBr (1.90 g, 11.1089 mmol) was added to the reaction mixture. The reaction mixture was stirred for 9 h at 20° C. The reaction mixture was diluted with EA (150 mL) and washed with water (150 mL) and brine (2×150 mL). The organics dried over Na₂SO₄, filtered and evaporated under reduced pressure. The residue was purified by flash chromatography, eluting with ethyl acetate in n-hexane (0-10%). The pure fraction was concentrated and dried under reduced pressure. There was tert-butyl 5-benzyloxy-6-bromo-4-fluoro-isoindoline-2-carboxylate (3.43 g, 8.1224 mmol, 99.93% yield) obtained as a white semi-solid. LCMS: (ESI, m/z): [M-tBu+ACN]⁺=407.050. ¹H NMR (400 MHz, CDCl₃-d) 6 7.54 (d, J=7.3 Hz, 2H), 7.45-7.35 (m, 3H), 7.27 (s, 0.5H), 7.20 (s, 0.5H), 5.13 (s, 2H), 4.70 (s, 1H), 4.65 (s, 2H), 4.62 (s, 1H), 1.54 (s, 9H).

### Step f: tert-butyl 5-benzyloxy-4-fluoro-6-hydroxy-isoindoline-2-carboxylate

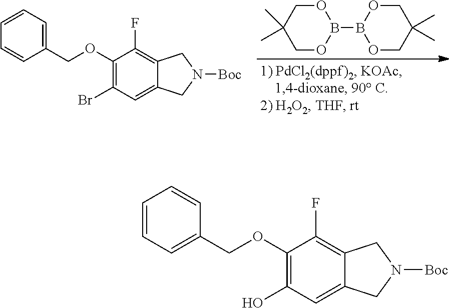

Pd(dppf)Cl₂ (1.12 g, 1.5307 mmol) was added to a solution of tert-butyl 5-benzyloxy-6-bromo-4-fluoro-isoindoline-2-carboxylate (3.30 g, 7.8146 mmol), bis(neopentylglycolato)diboron (5.46 g, 24.1716 mmol) and KOAc (2.32 g, 23.6392 mmol) in 1,4-Dioxane (80 mL) at 20° C. The reaction mixture was heated to 90° C. and stirred overnight under nitrogen atmosphere. The reaction mixture was diluted with EA (100 mL) and filtered through a celite. The filtrate was evaporated under reduced pressure. H₂O₂(88.1974 mmol, 10 mL, 30% aqueous solution) was added to a mixture of the residue and NaHCO₃ (11.9038 mmol, 20 mL, 5% aqueous solution) in THF (80 mL) at 0° C. The reaction mixture was stirred for 2 h at 20° C. The resulting reaction mixture was diluted with brine (150 mL) and saturated NaHSO₃ (100 mL), and extracted with EA (200 mL). The organic layer was collected and dried over Na₂SO₄, filtered and evaporated under reduced pressure. The residue was purified on silica gel column chromatography, eluting with ethyl acetate/n-hexane (0-10%). There was tert-butyl 5-benzyloxy-4-fluoro-6-hydroxy-isoindoline-2-carboxylate (1.93 g, 5.3702 mmol, 68.93% yield) obtained as an off-white solid. LCMS: (ESI, m/z): [M−H]⁻=358.100. ¹H NMR (400 MHz, CDCl₃-d) 6 7.46-7.36 (m, 5H), 6.62 (s, 0.5H), 6.58 (s, 0.5H), 5.15 (s, 2H), 4.68 (s, 1H), 4.65 (s, 1H), 4.61 (s, 1H), 4.58 (s, 1H), 1.54 (s, 9H).

### Step g: tert-butyl 5-benzyloxy-4-fluoro-6-methoxy-isoindoline-2-carboxylate

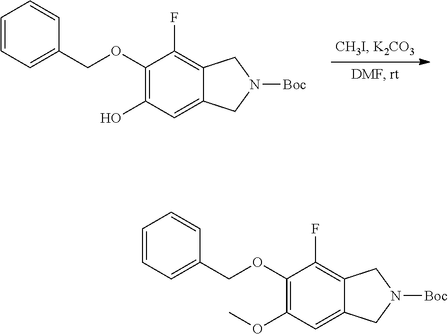 [SMILES: CC(C)(C)OC(=O)N1Cc2cc(O)c(OCc3ccccc3)c(F)c2C1 ; COc1cc2c(c(F)c1OCc1ccccc1)CN(C(=O)OC(C)(C)C)C2]

K₂CO₃ (1.18 g, 8.5380 mmol) was added to a solution of tert-butyl 5-benzyloxy-4-fluoro-6-hydroxy-isoindoline-2-carboxylate (1.9 g, 5.2867 mmol) in DMF (25 mL) at 0° C. After the mixture was stirred for 30 min at 0° C., CH₃I (1.12 g, 7.8908 mmol) was added. The reaction mixture was stirred for 3 h at 20° C. The resulting reaction mixture was diluted with water (200 mL) and extracted with EA (200 mL). The organic layer was separated and washed with brine (2×150 mL). The organic layer was collected and dried over Na₂SO₄, filtered and evaporated under reduced pressure. There was tert-butyl 5-benzyloxy-4-fluoro-6-methoxy-isoindoline-2-carboxylate (1.98 g, 5.3024 mmol, 100% yield) obtained as a colorless oil, which was directly used in the next step without purification. LCMS: (ESI, m/z): [M-tBu+ACN]⁺=359.150. ¹H NMR (400 MHz, CDCl₃-d) 6 7.39 (d, J=7.4 Hz, 2H), 7.31-7.21 (m, 3H), 6.51 (s, 1H), 4.99 (s, 2H), 4.55 (s, 4H), 3.77 (s, 3H), 1.44 (s, 9H).

### Step h: tert-butyl 4-fluoro-5-hydroxy-6-methoxy-isoindoline-2-carboxylate

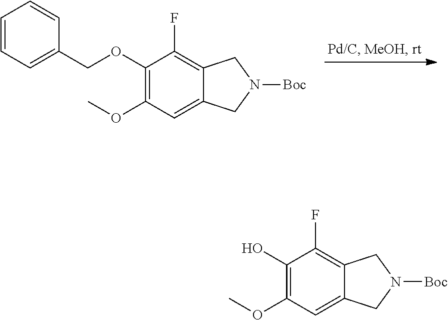 [SMILES: COc1cc2c(c(F)c1OCc1ccccc1)CN(C(=O)OC(C)(C)C)C2 ; COc1cc2c(c(F)c1O)CN(C(=O)OC(C)(C)C)C2]

10% Pd/C (1.98 g, contain 55% H₂O) was added to a solution of tert-butyl 5-benzyloxy-4-fluoro-6-methoxy-isoindoline-2-carboxylate (1.96 g, 5.2488 mmol) in MeOH (40 mL) and THF (10 mL) at 20° C. The reaction mixture was stirred for 5 h at 20° C. under H₂ atmosphere. The reaction mixture was diluted with EA (20 mL) and filtered through a celite. The filtrate was evaporated under reduced pressure. The residue was purified on silica gel column chromatography, eluting with ethyl acetate/n-hexane (0-40%). The pure fraction was concentrated and dried under reduced pressure. There was tert-butyl 4-fluoro-5-hydroxy-6-methoxy-isoindoline-2-carboxylate (1.21 g, 4.2712 mmol, 80.67% yield) obtained as a brown semi-solid. LCMS: (ESI, m/z): [M−H]⁻=282.100. ¹H NMR (400 MHz, CDCl₃-d) 6 6.58 (s, 1H), 4.67 (s, 2H), 4.63 (s, 2H), 3.92 (s, 3H), 1.53 (s, 9H).

### INT A11: ethyl 4-(4-fluoro-5-hydroxy-6-methoxy-isoindolin-2-yl)-4-oxo-butanoate

### Step a: 7-fluoro-6-methoxy-2-[(4methoxyphenyl) methyl] isoindolin-1-one

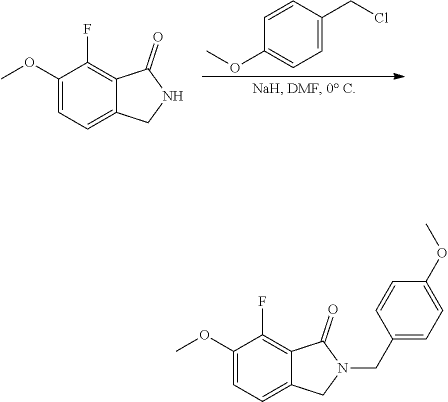

4-Methoxybenzylchloride (4.141 g, 26.4416 mmol) was added to a solution of NaH (1.000 g, 25.0024 mmol) and 7-fluoro-6-methoxyisoindolin-1-one (4.336 g, 23.9342 mmol) in DMF (100 mL) at 0° C. under N₂ atmosphere. The reaction mixture was stirred for 3 h and warmed up to 20° C. naturally. The reaction mixture was quenched with adding to water (300 mL) at 20° C., extracted with EA (3×200 mL) and washed with brine (150 mL). The organics dried over Na₂SO₄, filtered and evaporated under reduced pressure. The residue was purified on silica gel column EA/n-Hex (0-100%). The pure fraction was concentrated and dried under vacuo. There was 7-fluoro-6-methoxy-2-[(4methoxyphenyl) methyl] isoindolin-1-one (6.51 g, 21.6055 mmol, 90.2706% yield) obtained as a yellow solid. LCMS: (ESI, m/z): [M+H]⁺=302.110. ¹H NMR (400 MHz, DMSO-d₆) δ 7.39 (t, J=7.9 Hz, 1H), 7.27 (d, J=8.2 Hz, 1H), 7.22 (d, J=8.2 Hz, 2H), 6.91 (d, J=8.3 Hz, 2H), 4.60 (s, 2H), 4.25 (s, 2H), 3.87 (s, 3H), 3.73 (s, 3H).

### Step b: 4-fluoro-5-methoxy-2[(4methoxyphenyl)methyl]isoindoline

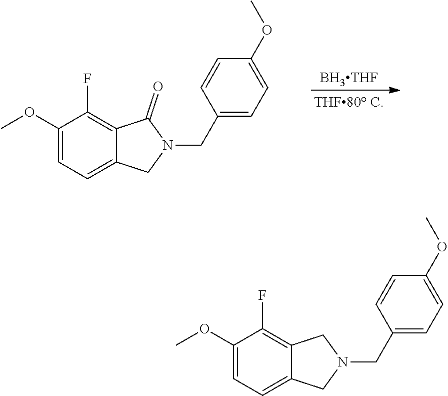 [SMILES: COc1ccc(CN2Cc3ccc(OC)c(F)c3C2=O)cc1 ; COc1ccc(CN2Cc3ccc(OC)c(F)c3C2)cc1 ; "BH3·THF"]

Borane-tetrahydrofuran complex (120 mL, 120 mmol) was added to a solution of 7-fluoro-6-methoxy-2-(4-methoxybenzyl)isoindolin-1-one (6.17 g, 20.4771 mmol) in THF (60 mL) at 20° C. under N₂ atmosphere. The reaction mixture was heated to 80° C. and stirred for 5 h. The reaction mixture was quenched with pouring into MeOH (300 mL) at 20° C. and evaporated under reduced pressure. The residue was diluted with MeOH (100 mL). The precipitate was collected by filtration, washed with MeOH (50 mL). The filter cake was dried under vacuo. There was 4-fluoro-5-methoxy-2[(4methoxyphenyl)methyl]isoindoline (3.854 g, 13.4132 mmol, 65.5036% yield) obtained as a white solid. LCMS: (ESI, m/z): [M+H]⁺=288.130. ¹H NMR (400 MHz, DMSO-d₆) δ 7.41 (d, J=8.0 Hz, 2H), 7.06-6.93 (m, 2H), 6.88 (d, J=8.0 Hz, 2H), 4.53-4.37 (m, 2H), 4.22-4.08 (m, 4H), 3.79 (s, 3H), 3.74 (s, 3H).

### Step c: ethyl 4-(4-fluoro-5-methoxy-isoindolin-2-yl)-4-oxo-butanoate

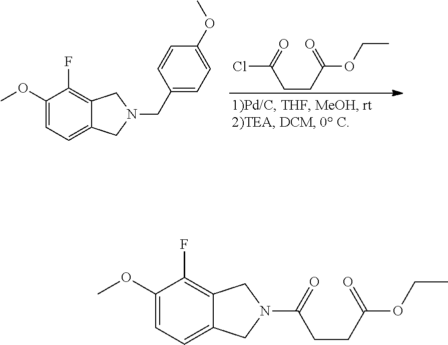

Pd/C (2.17 g, 2.0391 mmol) was added to a solution of 4-fluoro-5-methoxy-2-(4-methoxybenzyl)isoindoline (3.36 g, 11.6940 mmol) in THF (20 mL) and MeOH (20 mL) at 20° C. The reaction mixture was stirred overnight at 20° C. under H₂ atmosphere. The precipitate was collected by filtration, washed with MeOH (50 mL). The filtrate was evaporated under reduced pressure. Ethyl Succinyl Chloride (5.05 g, 30.6830 mmol) was added to a solution of the residue and TEA (3.91 g, 38.6404 mmol) in DCM (50 mL) at 0° C. under N₂ atmosphere. The reaction mixture was stirred for 1 h at 20° C. The reaction mixture was quenched with adding of water (50 mL) at 20° C., extracted with DCM (3×50 mL) and washed with brine (50 mL). The organics dried over Na₂SO₄, filtered and evaporated under reduced pressure. The residue was purified on silica gel column EA/n-Hex (0-45%). The pure fraction was concentrated and dried under vacuo. There was ethyl 4-(4-fluoro-5-methoxy-isoindolin-2-yl)-4-oxo-butanoate (2.619 g, 8.8688 mmol, 75.8406% yield) obtained as a yellow solid. LCMS: (ESI, m/z): [M+H]⁺=296.120. ¹H NMR (400 MHz, DMSO-d₆) δ 7.16-7.09 (m, 2H), 4.91 (s, 1H), 4.81 (s, 1H), 4.64 (s, 1H), 4.58 (s, 1H), 4.05 (q, J=7.2 Hz, 2H), 3.84 (s, 3H), 2.73-2.53 (m, 4H), 1.18 (t, J=7.2 Hz, 3H).

### Step d: ethyl 4-(4-fluoro-5-hydroxy-isoindolin-2-yl)-4-oxo-butanoate

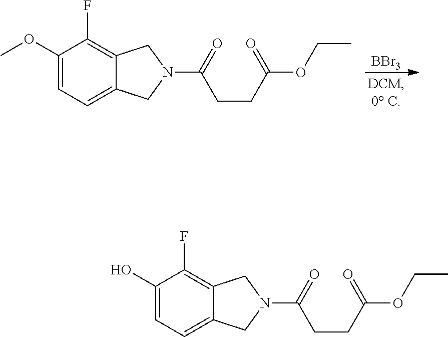

Tribromoboron (20 mL, 20 mmol) was added to a solution of ethyl 4-(4-fluoro-5-methoxyisoindolin-2-yl)-4-oxobutanoate (2.597 g, 8.7943 mmol) in DCM (40 mL) at 0° C. under N₂ atmosphere. The reaction mixture was stirred for 2 h at 20° C. The reaction mixture was quenched with adding to EtOH (100 mL) at 20° C., The reaction mixture was evaporated under reduced pressure and diluted with H₂O (200 mL), extracted with EA (2×100 mL) and washed with brine (100 mL). The organics dried over Na₂SO₄, filtered and evaporated under reduced pressure. There was ethyl 4-(4-fluoro-5-hydroxy-isoindolin-2-yl)-4-oxo-butanoate (2.408 g, 8.5609 mmol, 97.3461% yield) obtained as a yellow solid. LCMS: (ESI, m/z): [M+H]⁺=282.110. ¹H NMR (400 MHz, DMSO-d₆) δ 9.83 (s, 1H), 6.99-6.85 (m, 2H), 4.88 (s, 1H), 4.77 (s, 1H), 4.62 (s, 1H), 4.54 (s, 1H), 4.05 (q, J=7.2 Hz, 2H), 2.66-2.53 (m, 4H), 1.18 (t, J=7.2 Hz, 3H).

### Step e: ethyl 4-(6-bromo-4-fluoro-5-hydroxy-isoindolin-2-yl)-4-oxo-butanoate

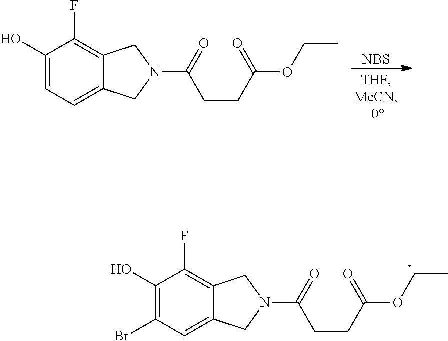

NBS (1.194 g, 6.7085 mmol) was added to a solution of ethyl 4-(4-fluoro-5-hydroxy-isoindolin-˜f)--oxo-butanoate (2.106 g, 7.4872 mmol) in MeCN (20 mL) and THF (20 mL) at 0° C. under N₂ atmosphere. The reaction mixture was stirred for 2 h and warmed up to 20° C. naturally. The precipitate was collected by filtration. The filter cake was dried under vacuo. There was ethyl 4-(6-bromo-4-fluoro-5-hydroxy-isoindolin-2-yl)-4-oxo-butanoate (2.84 g, 7.8851 mmol, 105.3134% yield) obtained as a white solid. LCMS: (ESI, m/z): [M+H]⁺=359.010. ¹H NMR (400 MHz, DMSO-d₆) δ 10.41 (s, 1H), 7.36 (s, 0.5H), 7.35 (s, 0.5H), 4.87 (s, 1H), 4.78 (s, 1H), 4.60 (s, 1H), 4.55 (s, 1H), 4.14-3.97 (q, J=7.2 Hz, 2H), 2.70-2.52 (m, 4H), 1.18 (t, J=7.2 Hz, 3H).

### Step f: ethyl 4-(6-bromo-4-fluoro-5-(methoxymethoxy)isoindolin-2-yl)-4-oxobutanoate

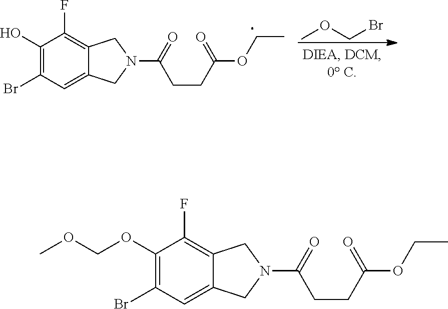

Bromomethyl methyl ether (1.20 g, 9.6028 mmol) was added to a solution of ethyl 4-(6-bromo-4-fluoro-5-hydroxy-isoindolin-2-yl)-4-oxo-butanoate (2.23 g, 6.1914 mmol) and DIEA (2.55 g, 19.7304 mmol) in DCM (50 mL) at 0° C. under N₂ atmosphere. The reaction mixture was stirred for 2 h and warmed up to 20° C. naturally. The reaction mixture was quenched with adding of water (50 mL) at 20° C., extracted with DCM (3×50 mL) and washed with brine (50 mL). The organics dried over Na₂SO₄, filtered and evaporated under reduced pressure. The residue was purified on C18 column ACN/H₂O (0.1% FA)(0-50%). The pure fraction was concentrated and dried under vacuo. There was ethyl 4-(6-bromo-4-fluoro-5-(methoxymethoxy)isoindolin-2-yl)-4-oxobutanoate (1.809 g, 4.4752 mmol, 72.2806% yield) obtained as a yellow solid. LCMS: (ESI, m/z): [M+H]⁺=403.140. H NMR (400 MHz, DMSO-d₆) δ 7.52 (s, 0.5H), 7.51 (s, 0.5H), 5.16 (s, 2H), 4.90 (s, 1H), 4.85 (s, 1H), 4.63 (s, 1H), 4.61 (s, 1H), 4.05 (q, J=7.2 Hz, 2H), 3.54 (s, 3H), 2.72-2.53 (m, 4H), 1.24-1.12 (t, J=7.2 Hz, 3H).

### Step g: ethyl 4-(4-fluoro-6-hydroxy-5-(methoxymethoxy)isoindolin-2-yl)-4-oxobutanoate

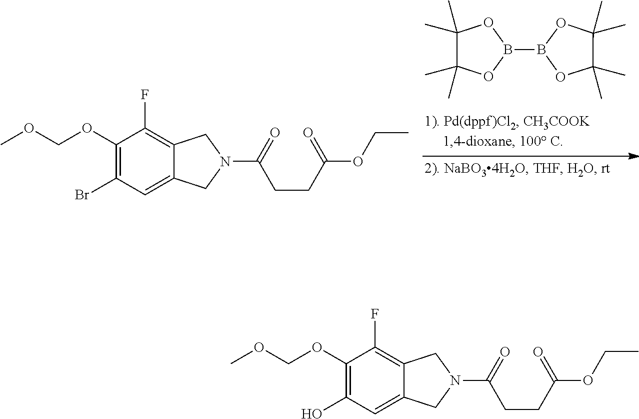

Pd(dppf)Cl₂ (0.228 g, 311.6016 mol), 4, 4,4′,4′,5,5,5′,5′-Octamethyl-2,2′-bi(1,3,2-dioxaborolane) (1.170 g, 4.6074 mmol) and Potassium Acetate (0.743 g, 7.5706 mmol) was added to a solution of ethyl 4-(6-bromo-4-fluoro-5-(methoxymethoxy)isoindolin-2-yl)-4-oxobutanoate (0.735 g, 1.8183 mmol) in 1,4-Dioxane (20 mL) at 20° C. under N₂ atmosphere. The reaction mixture was heated to 100° C. and stirred for 12 h. The reaction mixture was quenched with adding of water (150 mL) at 20° C., extracted with EA (3×150 mL) and washed with brine (50 mL). The organics dried over Na₂SO₄, filtered and evaporated under reduced pressure. Sodium perborate (0.91 g, 5.9145 mmol) was added to a solution of the residue in THF (10 mL) and H₂O (10 mL) at 20° C. under N₂ atmosphere. The reaction mixture was stirred for 1 h at 20° C. The reaction mixture was evaporated under reduced pressure. The residue was purified on C18 column ACN/H₂O (0.1% FA)(0-38%). The pure fraction was concentrated and dried under vacuo. There was ethyl 4-(4-fluoro-6-hydroxy-5-(methoxymethoxy)isoindolin-2-yl)-4-oxobutanoate (0.365 g, 1.0693 mmol, 58.8% yield) obtained as a white solid. LCMS: (ESI, m/z): [M+H]⁺=342.130. ¹H NMR (400 MHz, DMSO-d₆) δ 9.97 (s, 1H), 6.68 (s, 1H), 5.04 (s, 2H), 4.80 (s, 1H), 4.76 (s, 1H), 4.55 (s, 1H), 4.53 (s, 1H), 4.05 (q, J=7.2 Hz, 2H), 3.47 (s, 3H), 2.67-2.51 (m, 4H), 1.18 (t, J=7.2 Hz, 3H).

### Step h: ethyl 4-(4-fluoro-5-hydroxy-6-methoxy-isoindolin-2-yl)-4-oxo-butanoate

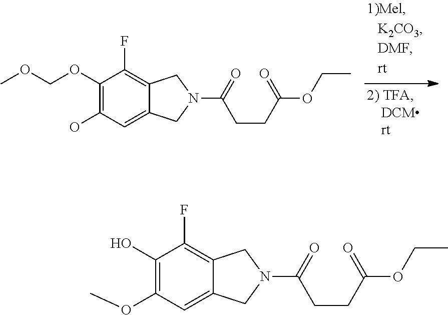

Methyl Iodidle (0.345 g, 2.4306 mmol) was added to a mixture of Potassium carbonate (0.448 g, 3.2415 mmol) and ethyl 4-(4-fluoro-6-hydroxy-5-(methoxymethoxy)isoindolin-2-yl)-4-oxobutanoate (0.328 g, 960.9441 μmol) in DMF (10 mL) at 20° C. under N₂ atmosphere. The reaction mixture was stirred for 1 h at 20° C. The reaction mixture was quenched with adding of water (50 mL) at 20° C., extracted with DCM (3×50 mL) and washed with brine (50 mL). The organics dried over Na₂SO₄, filtered and evaporated under reduced pressure. Trifluoroacetic acid (2 mL) was added to a solution of the residue (0.446 g, 1.2551 mmol) in DCM (5 mL) at 20° C. under N₂ atmosphere. The reaction mixture was stirred for 2 h at 20° C. The reaction mixture was evaporated under reduced pressure. The residue was purified on C18 column ACN/H₂O (0-80%). The pure fraction was concentrated and dried under vacuo. There was ethyl 4-(4-fluoro-5-hydroxy-6-methoxy-isoindolin-2-yl)-4-oxo-butanoate (0.274 g, 880.1663 mol, 91.6% yield) obtained as a white solid. LCMS: (ESI, m/z): [M+H]⁺=312.12.

¹H NMR (400 MHz, DMSO-d₆) δ 9.17 (s, 1H), 6.83 (s, 0.5H), 6.80 (s, 0.5H), 4.82 (s, 1H), 4.77 (s, 1H), 4.57 (s, 1H), 4.54 (s, 1H), 4.05 (q, J=7.2 Hz, 2H), 3.80 (d, J=2.0 Hz, 3H), 2.68-2.51 (m, 4H), 1.18 (t, J=7.2 Hz, 3H).

### INT A12: ethyl 4-(6-hydroxy-5-methoxythieno[3,2-b]pyridin-2-yl)-4-oxobutanoate

### Step a: 6-bromo-5-methoxythieno[3,2-b]pyridine

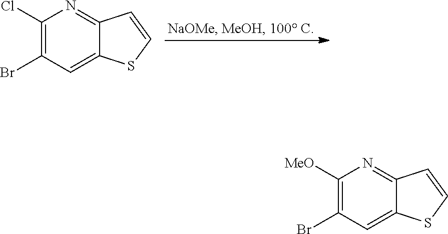 [SMILES: Clc1nc2ccsc2cc1Br ; COc1nc2ccsc2cc1Br]

A solution of 6-bromo-5-chlorothieno[3,2-b]pyridine (1.0 g, 4.0 mmol) and NaOMe (9.3 ml, 40 mmol) in MeOH (6 mL) was stirred at 100° C. for 1 h under N₂ atmosphere in microwave irradiation reactor. 1 N citric acid (80 mL) was added, then extracted with EtOAc (70 mL×3). The combined organic solution was dried over Na₂SO₄, concentrated to give 6-bromo-5-methoxythieno[3,2-b]pyridine (1.0 g, 4.0 mmol, 99% yield) as a brown solid. ¹H NMR (300 MHz, DMSO-d₆): δ 8.75 (d, J=0.5 Hz, 1H), 8.08 (d, J=5.5 Hz, 1H), 7.42 (dd, J=5.5, 0.5 Hz, 1H), 3.98 (s, 3H). LCMS: (ESI, m/z): [M+H]⁺=244.0.

### Step b: 6-bromo-5-methoxythieno[3,2-b]pyridine-2-carboxylic acid

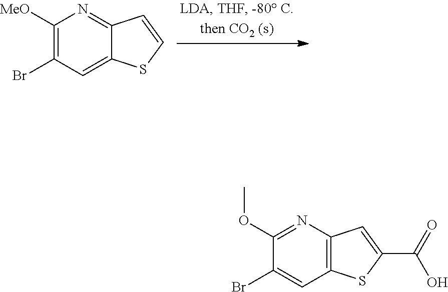

To a solution of 6-bromo-5-methoxythieno[3,2-b]pyridine (5.0 g, 20.0 mmol) in THF (200 mL) was added LDA (20 ml, 40 mmol) at −80° C. The reaction mixture was stirred at −80° C. for 30 min under N₂ atmosphere. Then the solution was poured onto CO₂ solid. The reaction mixture was stirred for about 1 h. 1 N HCl (50 mL) and water (200 ml) was added, then extracted with EtOAc (70 mL×3). The combined organic solution was dried over Na₂SO₄, concentrated to give 6-bromo-5-methoxythieno[3,2-b]pyridine-2-carboxylic acid (4.9 g, 17.0 mmol, 85% yield) as a brown solid. ¹H NMR (300 MHz, DMSO-d₆): δ 8.84 (s, 1H), 7.92 (s, 1H), 4.00 (s, 3H). LCMS: (ESI, m/z): [M+H]⁺=287.9.

### Step c: tert-butyl 3-(6-bromo-5-methoxythieno[3,2-b]pyridin-2-yl)-3-oxopropanoate

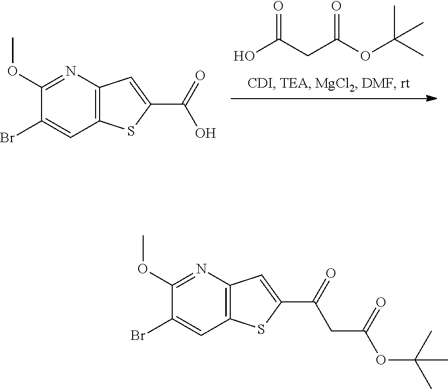 [SMILES: COc1nc2cc(C(=O)O)sc2cc1Br ; CC(C)(C)OC(=O)CC(=O)O ; COc1nc2cc(C(=O)CC(=O)OC(C)(C)C)sc2cc1Br]

To a solution of 6-bromo-5-methoxythieno[3,2-b]pyridine-2-carboxylic acid (4.9 g, 17.0 mmol) in DMF (147 mL) was added CDI (5.5 g, 34.0 mmol). After stirring at RT for 2 h under N₂ atmosphere, 3-tert-Butoxy-3-oxopropanoic acid (5.5 g, 34.0 mmol), TEA (5.2 g, 51.0 mmol, 3.0 eq) and MgCl₂ (3.3 g, 34.0 mmol) was added, then stirred at RT for overnight. Upon completion, sat. NaHCO₃ solution (200 mL) was added, then extracted with EtOAc (200 mL×3). The combined organic solution was dried over Na₂SO₄, concentrated to give a residue, purified by column chromatography (Petroleum ether/Ethyl acetate=20:1) to give tert-butyl 3-(6-bromo-5-methoxythieno[3,2-b]pyridin-2-yl)-3-oxopropanoate (5.0 g, 12.9 mmol, 62% yield) as a yellow solid.

¹H NMR (300 MHz, DMSO-d₆): δ 8.87 (s, 1H), 8.33 (s, 1H), 4.17 (s, 2H), 4.01 (s, 3H), 1.40 (s, 9H).

LCMS: (ESI, m/z): [M+H]⁺=386.0.

### Step d: 1-(tert-butyl) 4-ethyl 2-(6-bromo-5-methoxythieno[3,2-b]pyridine-2-carbonyl)succinate

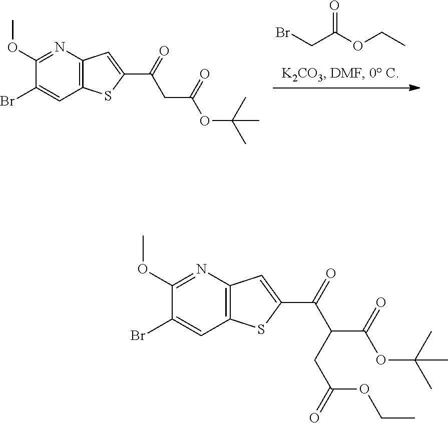

To a solution of tert-butyl 3-(6-bromo-5-methoxythieno[3,2-b]pyridin-2-yl)-3-oxopropanoate (3.0 g, 7.8 mmol) in DMF (60 mL) was added K₂CO₃ (1.8 g, 12.5 mmol) and Ethyl bromoacetate (1.3 g, 78 mmol) at 0° C.

The reaction mixture was stirred at 0° C. for 5 h under N₂ atmosphere. NaHCO₃ solution (100 mL) was added, then extracted with EtOAc (100 mL×3). The combined organic solution was dried over Na₂SO₄, concentrated to give a residue, purified by column chromatography (Petroleum ether/Ethyl acetate=20:1) to give 1-(tert-butyl) 4-ethyl 2-(6-bromo-5-methoxythieno[3,2-b]pyridine-2-carbonyl)succinate (3.3 g, 7.0 mmol, 89% yield) as a white solid.

¹H NMR (300 MHz, DMSO-d₆): δ 8.89 (s, 1H), 8.47 (s, 1H), 5.00 (t, J=7.3 Hz, 1H), 4.12-3.93 (m, 5H), 2.94 (t, J=7.3 Hz, 2H), 1.31 (d, J=4.5 Hz, 9H), 1.13 (t, J=7.1 Hz, 3H).

LCMS: (ESI, m/z): [M+H]⁺=472.0.

### Step e: ethyl 4-(6-bromo-5-methoxythieno[3,2-b]pyridin-2-yl)-4-oxobutanoate

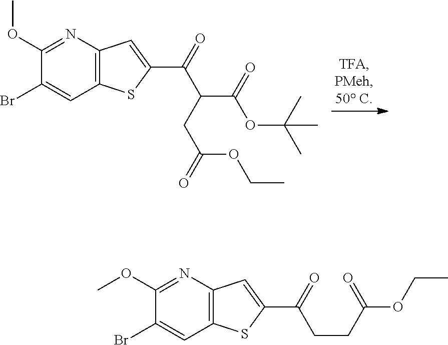

To a solution of 1-(tert-butyl) 4-ethyl 2-(6-bromo-5-methoxythieno[3,2-b]pyridine-2-carbonyl)succinate (3.3 g, 7.0 mmol) in PhMe (66 mL) was added TFA (33 ml). After stirring at 50° C. for 1.5 h under N₂ atmosphere, the solution was concentrated to give a residue, and the obtained solid was triturated with Petroleum ether/ethyl acetate (10/1) to obtain crude compound ethyl 4-(6-bromo-5-methoxythieno[3,2-b]pyridin-2-yl)-4-oxobutanoate (2.2 g, 5.9 mmol, 84% yield) as a brown solid. ¹H NMR (300 MHz, DMSO-d₆): δ 8.87 (s, 1H), 8.37 (s, 1H), 4.11-3.98 (m, 5H), 3.45-3.36 (m, 2H), 2.71-2.63 (m, 2H), 1.17 (t, J=7.1 Hz, 3H).

LCMS: (ESI, m/z): [M+H]⁺=372.0.

### Step f: (2-(4-ethoxy-4-oxobutanoyl)-5-methoxythieno[3,2-b]pyridin-6-yl)boronic acid

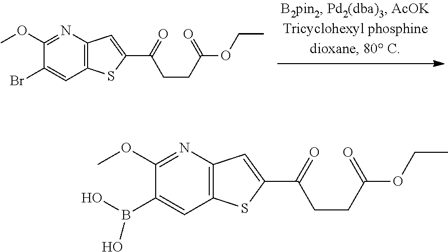 [SMILES: CCOC(=O)CCC(=O)c1cc2nc(OC)c(Br)cc2s1 ; CCOC(=O)CCC(=O)c1cc2nc(OC)c(B(O)O)cc2s1]

Ethyl 4-(6-bromo-5-methoxythieno[3,2-b]pyridin-2-yl)-4-oxobutanoate (1.6 g, 4.3 mmol), B₂Pin₂ (1.4 g, 5.3 mmol), AcOK (688 mg, 6.9 mmol), Pd2(dba)₃ (112 mg, 0.1 mmol) and Tricyclohexyl phosphine (128 mg, 0.43 mmol) were placed in the reaction bottle. The bottle was evacuated and filled with argon for three times. Then dioxane (32 mL) was added at room temperature. The mixture was stirred at 80° C. for 1 h. Water (50 mL) was added to the reaction mixture, extracted with EtOAc (3×50 mL), and the combined organic layers were washed with saturated brine and dried over anhydrous sodium sulfate. The solution was concentrated to give (2-(4-ethoxy-4-oxobutanoyl)-5-methoxythieno[3,2-b]pyridin-6-yl)boronic acid (3.2 g) a crude residue. It was used for next step without any further purification. LCMS: (ESI, m/z): [M+H]⁺=338.1.

### Step g: ethyl 4-(6-hydroxy-5-methoxythieno[3,2-b]pyridin-2-yl)-4-oxobutanoate

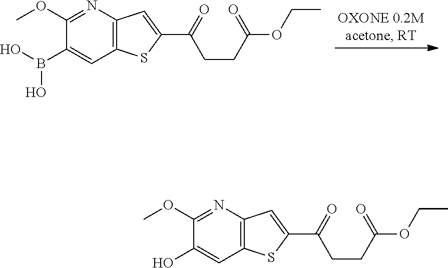

To a solution of (2-(4-ethoxy-4-oxobutanoyl)-5-methoxythieno[3,2-b]pyridin-6-yl)boronic acid (3.2 g, 4.3 mmol, crude) in acetone (48 mL) was added OXONE (36 ml, 6.5 mmol). After stirring at RT for 2 h. Sat. NaHSO₃₋solution (120 mL) was added, after stirring at RT for 1 h, the mixture was extracted with EtOAc (3×30 mL), and the combined organic layers were washed with saturated brine and dried over anhydrous sodium sulfate. The solution was concentrated to give a crude residue, purified by column chromatography (Petroleum ether/Ethyl acetate=10:1 to 5:1) to give ethyl 4-(6-hydroxy-5-methoxythieno[3,2-b]pyridin-2-yl)-4-oxobutanoate (340 mg, 1.1 mmol, 25% yield) as a yellow solid. ¹H NMR (400 MHz, DMSO-d₆): δ 10.44 (s, 1H), 8.22 (s, 1H), 7.65 (s, 1H), 4.05 (q, J=7.1 Hz, 2H), 3.97 (s, 3H), - 3.30-3.31 (br, 2 h), 2.65 (t, J=6.1 Hz, 2H), 1.17 (t, J=7.1 Hz, 3H).

LCMS: (ESI, m/z): [M+H]⁺=310.1.

The following intermediates were synthesized with the above steps or improved procedure with the corresponding starting materials and intermediates.

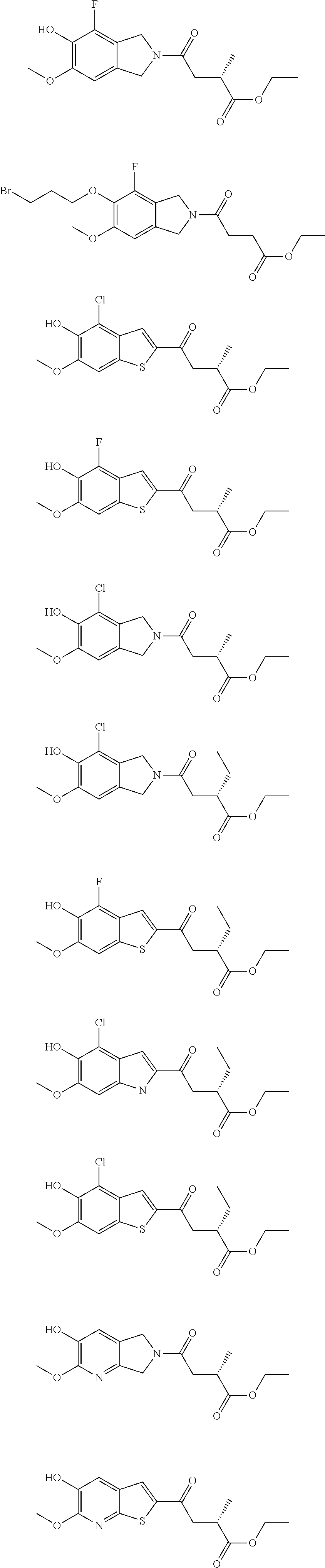

### EXAMPLE 11-1

### 4-(5-(3-((2-(3-carboxypropanoyl)-6-methoxybenzo[b]thiophen-5-yl)oxy)propoxy)-6-methoxyisoindolin-2-yl)-4-oxobutanoic acid

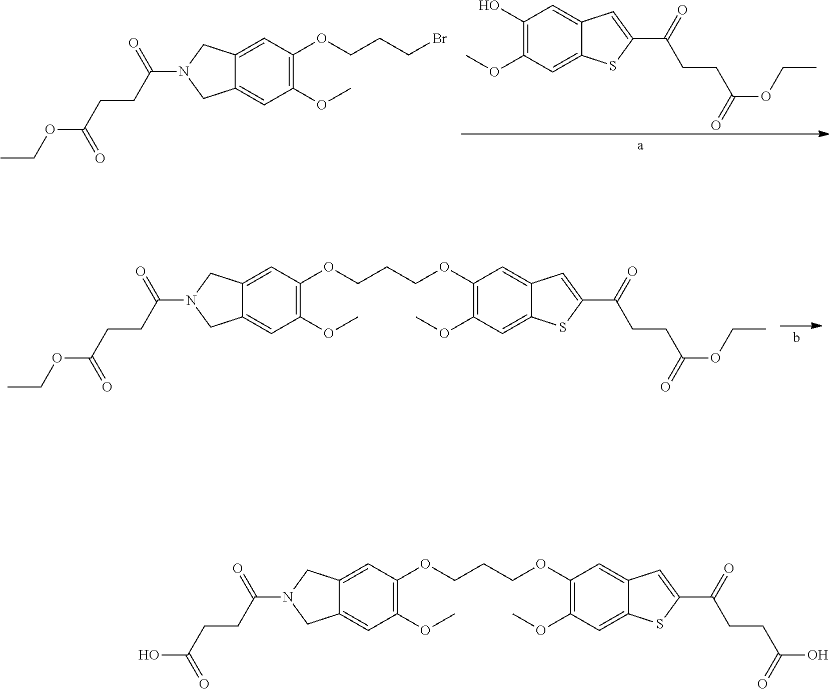

Step a: Potassium carbonate (0.240 g, 1.7365 mmol) was added to a solution of ethyl 4-(5-(3-bromopropoxy)-6-methoxyisoindolin-2-yl)-4-oxobutanoate (0.240 g, 579.3045 mol) and ethyl 4-(5-hydroxy-6-methoxybenzo[b]thiophen-2-yl)-4-oxobutanoate (0.169 g, 576.1737 gmol) in N,N-Dimethylformamide (10 mL) at 20° C. The reaction mixture was heated to 50° C. and stirred for 3 h. The reaction mixture was evaporated under reduced pressure. The reaction mixture was concentrated and diluted with EA (500 mL), washed with NaHCO₃ aq. (2×300 mL) and brine (200 mL). The organics was dried over Na₂SO₄, filtered and evaporated under reduced pressure. The residue was purified on silica gel column EA/Hept (0-100%). The pure fraction was concentrated and dried under vacuo. There was ethyl 4-(5-(3-((2-(4-ethoxy-4-oxobutanoyl)-6-methoxybenzo[b]thiophen-5-yl)oxy)propoxy)-6-methoxyisoindolin-2-yl)-4-oxobutanoate (0.147 g, 229.0694 mol, 62.8484% yield) obtained as a yellow solid.

¹H NMR (400 MHz, DMSO-d₆) δ 8.18 (s, 1H), 7.61 (d, J=1.9 Hz, 1H), 7.53 (s, 1H), 6.98 (dd, J=15.7, 11.9 Hz, 2H), 4.73 (d, J=9.4 Hz, 2H), 4.52 (s, 2H), 4.17 (dd, J=30.4, 7.5 Hz, 4H), 4.05 (qd, J=7.1, 3.4 Hz, 4H), 3.86 (d, J=1.6 Hz, 3H), 3.75 (d, J=2.0 Hz, 3H), 2.70-2.53 (m, 6H), 2.30-2.15 (m, 2H), 1.24-1.11 (m, 6H).

Step b: LiOH (0.022 g, 918.6456 mol) was added to a solution of ethyl 4-(5-(3-((2-(4-ethoxy-4-oxobutanoyl)-6-methoxybenzo[b]thiophen-5-yl)oxy)propoxy)-6-methoxyisoindolin-2-yl)-4-oxobutanoate in Tetrahydrofuran (10 mL) and Water (2 mL) at 20° C. The reaction mixture was stirred for 2 h. The reaction mixture was evaporated under reduced pressure and diluted with water and adjusted pH=3 with HCl (1 M). The precipitate was filtered. Filter cake was washed with water (20 mL). The filtrate cake was dried under vacuo at 60° C. There was 4-(5-(3-((2-(3-carboxypropanoyl)-6-methoxybenzo[b]thiophen-5-yl)oxy)propoxy)-6-methoxyisoindolin-2-yl)-4-oxobutanoic acid (0.119 g, 203.2031 mol, 90.5562% yield) obtained as a yellow solid.

¹H NMR (400 MHz, DMSO-d₆) δ 12.12 (s, 2H), 8.17 (s, 1H), 7.67-7.55 (m, 1H), 7.53 (s, 1H), 6.98 (dd, J=16.6, 12.5 Hz, 2H), 4.73 (d, J=8.2 Hz, 2H), 4.53 (s, 2H), 4.18 (ddd, J=23.1, 13.6, 6.3 Hz, 4H), 3.86 (d, J=1.0 Hz, 3H), 3.75 (d, J=1.9 Hz, 3H), 3.25 (d, J=6.6 Hz, 2H), 2.67-2.53 (m, 4H), 2.30-2.11 (m, 2H).

### Example 11-2

### 4-(5-(3-((2-(3-carboxypropanoyl)-4-chloro-6-methoxyisoindolin-5-yl)oxy)propoxy)-4-fluoro-6-methoxybenzo[b]thiophen-2-yl)-4-oxobutanoic acid

### Step a: 01-tert-butyl 04-ethyl 2-(5-bromo-4-fluoro-6-methoxy-benzothiophene-2-carbonyl)butanedioate

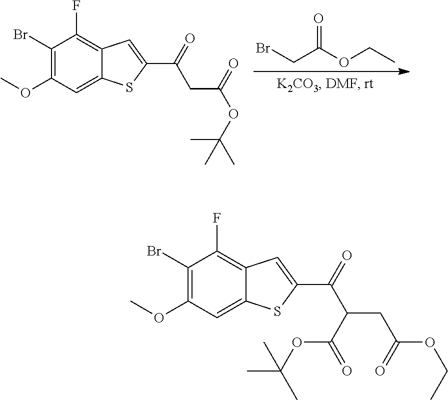

To a stirred solution of tert-butyl 3-(5-bromo-4-fluoro-6-methoxy-benzothiophen-2-yl)-3-oxo-propanoate (1.58 g, 3.9180 mmol) in N,N-Dimethylformamide (10 mL) was added K₂CO₃ (1.10 g, 7.9592 mmol) and ethyl bromoacetate (0.72 g, 4.3114 mmol) at 20° C. The resulting mixture was stirred for 2 h at 20° C. under nitrogen atmosphere. The reaction mixture was concentrated and diluted with EA (200 mL), washed with water (100 mL) and brine (50 mL). The organics dried over Na₂SO₄, filtered and evaporated under reduced pressure. The residue was purified by flash silica chromatography, elution gradient 0 to 100% Ethyl acetate in heptane. The pure fraction was concentrated and dried under vacuo. There was 01-tert-butyl 04-ethyl 2-(5-bromo-4-fluoro-6-methoxy-benzothiophene-2-carbonyl)butanedioate (1.925 g, 3.9338 mmol, 100.4016% yield) obtained as a yellow oil. LCMS: (ESI, m/z): [M+H]⁺=489.030.

### Step b: ethyl 4-(5-bromo-4-fluoro-6-methoxy-benzothiophen-2-yl)-4-oxo-butanoate

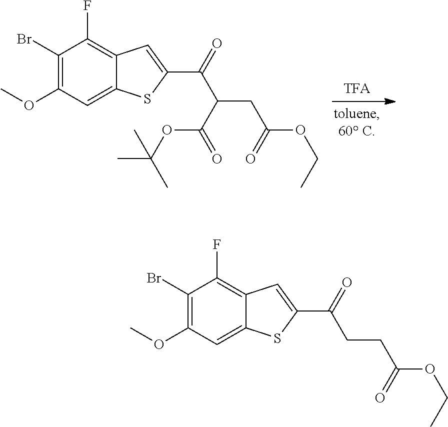 [SMILES: CCOC(=O)CC(C(=O)OC(C)(C)C)C(=O)c1cc2c(F)c(Br)c(OC)cc2s1 ; CCOC(=O)CCC(=O)c1cc2c(F)c(Br)c(OC)cc2s1]

Trifluoroacetic acid (2 mL) was added to a solution of 01-tert-butyl 04-ethyl 2-(5-bromo-4-fluoro-6-methoxy-benzothiophene-2-carbonyl)butanedioate (1.85 g, 3.7805 mmol) in Toluene (10 mL) at 20° C. The reaction mixture was stirred at 60° C. for 2 h. The reaction mixture was evaporated under reduced pressure. The residue was concentrated and diluted with DCM (200 mL), washed with NaHCO₃ aq (2×100 mL) and brine (100 mL). The organics dried over Na₂SO₄, filtered and evaporated under reduced pressure. The residue was purified by flash silica chromatography, elution gradient 0 to 100% Ethyl acetate in heptane. The pure fraction was concentrated and dried under vacuo. There was ethyl 4-(5-bromo-4-fluoro-6-methoxy-benzothiophen-2-yl)-4-oxo-butanoate (0.727 g, 1.8678 mmol, 49.4050% yield) obtained as a yellow solid. LCMS: (ESI, m/z): [M+H]⁺=388.978. ¹H NMR (400 MHz, DMSO-d₆) δ 8.39 (s, 1H), 7.71 (s, 1H), 4.06 (q, J=6.9 Hz, 2H), 3.98 (s, 3H), 3.39 (d, J=6.2 Hz, 2H), 2.73-2.62 (m, 2H), 1.18 (t, J=7.1 Hz, 3H).

### Step c: ethyl 4-(4-fluoro-6-methoxy-5-(4,4,5,5-tetramethyl-1,3,2-dioxaborolan-2-yl)benzo[b]thiophen-2-yl)-4-oxobutanoate

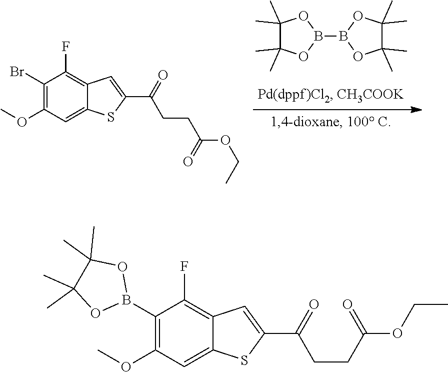

Pd(dppf)Cl₂ (0.15 g, 205.0010 mol) was added to a mixture of Potassium Acetate (0.60 g, 6.1136 mmol), 4,4,4′,4′,5,5,5′,5′-Octamethyl-2,2′-bi(1,3,2-dioxaborolane) (0.97 g, 3.8198 mmol) and ethyl 4-(5-bromo-4-fluoro-6-methoxy-benzothiophen-2-yl)-4-oxo-butanoate (0.72 g, 1.8498 mmol) in 1,4-Dioxane (30 mL) at 20° C. The reaction mixture was stirred at 100° C. for 20 h under N₂ atmosphere. The reaction mixture was evaporated under reduced pressure. The residue was concentrated and diluted with EA (200 mL), washed with NaHCO₃ aq (2×100 mL) and brine (100 mL). The organics dried over Na₂SO₄, filtered and evaporated under reduced pressure. The residue was purified by flash silica chromatography, elution gradient 0 to 100% Ethyl acetate in heptane. The pure fraction was concentrated and dried under vacuo. There was ethyl 4-(4-fluoro-6-methoxy-5-(4,4,5,5-tetramethyl-1,3,2-dioxaborolan-2-yl)benzo[b]thiophen-2-yl)-4-oxobutanoate (0.38 g, 870.9586 mol, 47.0845% yield) obtained as a white solid. LCMS: (ESI, m/z): [M+H]⁺=437.153.

### Step d: ethyl 4-(4-fluoro-5-hydroxy-6-methoxy-benzothiophen-2-yl)-4-oxo-butanoate

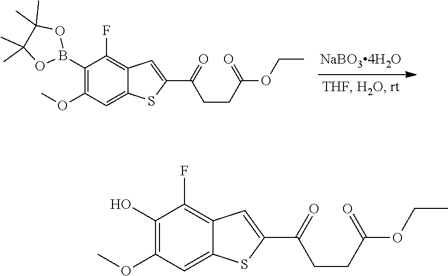

Sodium perborate tetrahydrate (0.21 g, 1.3649 mmol) was added to a solution of ethyl 4-(4-fluoro-6-methoxy-5-(4,4,5,5-tetramethyl-1,3,2-dioxaborolan-2-yl)benzo[b]thiophen-2-yl)-4-oxobutanoate in Tetrahydrofuran (10 mL) and water (10 mL) at 20° C. The reaction mixture was stirred at 20° C. for 1 h. The reaction mixture was concentrated and diluted with DCM (100 mL), washed with NaHCO₃ aq (2×500 mL) and brine (50 mL). The organics dried over Na₂SO₄, filtered and evaporated under reduced pressure. The residue was purified on silica gel column MeOH/DCM (0-5%). The pure fraction was concentrated and dried under vacuo.

There was ethyl 4-(4-fluoro-5-hydroxy-6-methoxy-benzothiophen-2-yl)-4-oxo-butanoate (0.263 g, 805.9094 mol, 92.5313% yield) obtained as a yellow solid. LCMS: (ESI, m/z): [M+H]⁺=327.062. H NMR (400 MHz, DMSO-d₆) δ 9.53 (s, 1H), 8.25 (s, 1H), 7.47 (s, 1H), 4.06 (q, J=7.1 Hz, 2H), 3.88 (s, 3H), 3.39-3.34 (m, 2H), 2.69-2.60 (m, 2H), 1.17 (t, J=7.1 Hz, 3H).

### Step e: ethyl 4-(4-chloro-5-hydroxy-6-methoxy-isoindolin-2-yl)-4-oxo-butanoate

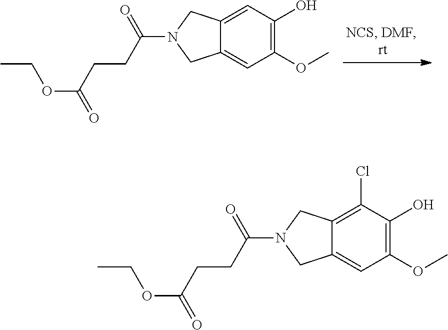

NCS (0.105 g, 786.3231 mol) was added to a solution of ethyl 4-(5-hydroxy-6-methoxyisoindolin-2-yl)-4-oxobutanoate (0.205 g, 698.9090 mol) in DMF (10 mL) at 0° C. under N₂ atmosphere. The reaction mixture was stirred for 12 h and warmed up to 20° C. naturally. The reaction mixture was purified on C18 column ACN/H₂O (0-25%). The pure fraction was concentrated and dried under vacuo. There was ethyl 4-(4-chloro-5-hydroxy-6-methoxy-isoindolin-2-yl)-4-oxo-butanoate (0.148 g, 451.5507 mol, 64.6079% yield) obtained as a white solid.

LCMS: (ESI, m/z): [M+H]⁺=328.100. ¹H NMR (400 MHz, DMSO-d₆) δ 9.41 (s, 1H), 6.97 (s, 0.5H), 6.95 (s, 0.5H), 4.81 (s, 1H), 4.76 (s, 1H), 4.59 (s, 1H), 4.51 (s, 1H), 4.05 (q, J=7.2 Hz, 2H), 3.82 (s, 3H), 2.69-2.53 (m, 4H), 1.18 (t, J=7.2 Hz, 3H).

### Step f: ethyl 4-[5-(3-bromopropoxy)-4-chloro-6-methoxy-isoindolin-2-yl]-4-oxo-butanoate

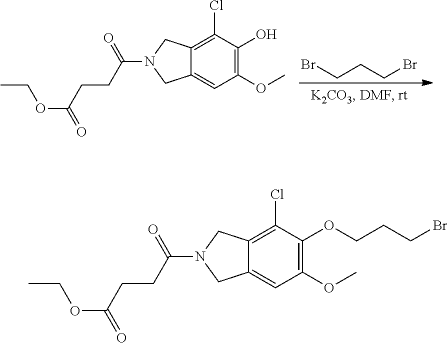

1,3-Dibromopropane (0.495 g, 2.4519 mmol) was added to a mixture of K₂CO₃ (0.199 g, 1.4399 mmol), ethyl 4-(4-chloro-5-hydroxy-6-methoxyisoindolin-2-yl)-4-oxobutanoate (0.149 g, 454.6023 mol) in DMF (5 mL) at 20° C. under N₂ atmosphere. The reaction mixture was stirred for 4 h at 20° C. The reaction mixture was purified on C18 column ACN/H₂O (0.1% FA)(0-50%). The pure fraction was concentrated and dried under vacuo. There was ethyl 4-(5-(3-bromopropoxy)-4-chloro-6-methoxyisoindolin-2-yl)-4-oxobutanoate (0.163 g, 363.2433 mol, 79.9035% yield) obtained as a white solid. LCMS: (ESI, m/z): [M+H]⁺=448.040. ¹H NMR (400 MHz, DMSO-d₆) δ 7.10 (s, 0.5H), 7.08 (s, 0.5H), 4.87 (s, 1H), 4.79 (s, 1H), 4.64 (s, 1H), 4.53 (s, 1H), 4.06-4.02 (m, 4H), 3.83 (d, J=2.4 Hz, 3H), 3.78-3.70 (m, 2H), 2.71-2.53 (m, 4H), 2.23-2.20 (m, 2H), 1.18 (t, J=7.2 Hz, 3H).

### Step g: ethyl 4-(5-(3-((4-chloro-2-(4-ethoxy-4-oxobutanoyl)-6-methoxyisoindolin-5-yl)oxy)propoxy)-4-fluoro-6-methoxybenzo[b]thiophen-2-yl)-4-oxobutanoate

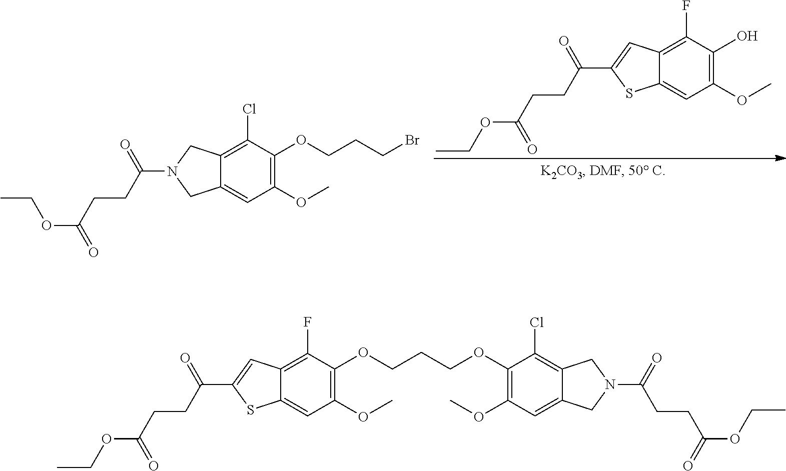

K₂CO₃ (0.115 g, 832.0942 mol) was added to a solution of ethyl 4-(5-(3-bromopropoxy)-4-chloro-6-methoxyisoindolin-2-yl)-4-oxobutanoate (0.068 g, 151.5371 mol), ethyl 4-(4-fluoro-5-hydroxy-6-methoxybenzo[b]thiophen-2-yl)-4-oxobutanoate (0.049 g, 150.1504 mol) in DMF (3 mL) at 20° C. under N₂ atmosphere. The reaction mixture was stirred for 4 h at 50° C. The reaction mixture was purified on C18 column ACN/H₂O (0.1% FA) (0-60%). The pure fraction was concentrated and dried by lyophilization. There was ethyl 4-(5-(3-((4-chloro-2-(4-ethoxy-4-oxobutanoyl)-6-methoxyisoindolin-5-yl)oxy)propoxy)-4-fluoro-6-methoxybenzo[b]thiophen-2-yl)-4-oxobutanoate (0.040 g, 57.6234 mol, 38.0259% yield) obtained as a white solid. LCMS: (ESI, m/z): [M+H]⁺=694.180. ¹H NMR (400 MHz, DMSO-d₆) δ 8.31 (s, 1H), 7.57 (s, 1H), 7.07 (s, 0.5H), 7.04 (s, 0.5H), 4.84 (s, 1H), 4.73 (s, 1H), 4.62 (s, 1H), 4.49 (s, 1H), 4.29 (t, J=6.0 Hz, 2H), 4.17 (t, J=6.0 Hz, 2H), 4.10-4.01 (m, 4H), 3.89 (s, 3H), 3.80 (d, J=2.0 Hz, 3H), 3.40-3.34 (m, 2H), 2.72-2.53 (m, 6H), 2.09-2.06 (m, 2H), 1.22-1.15 (m, 6H).

### Step h: 4-(5-(3-((2-(3-carboxypropanoyl)-4-chloro-6-methoxyisoindolin-5-yl)oxy)propoxy)-4-fluoro-6-methoxybenzo[b]thiophen-2-yl)-4-oxobutanoic

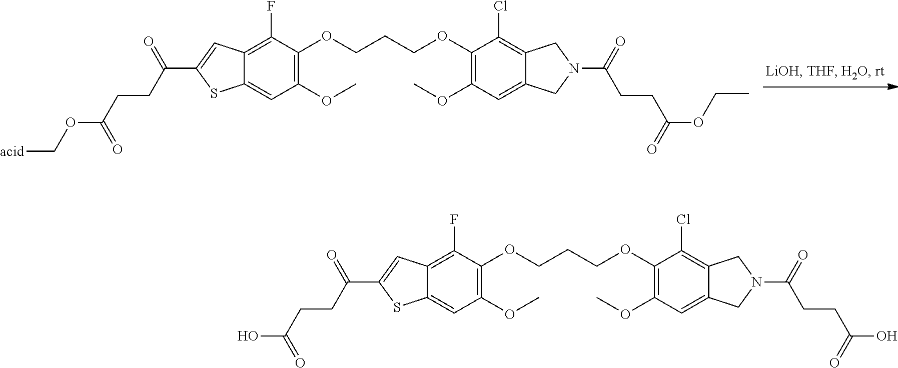

LiOH (0.048 g, 2.0043 mmol) was added to a solution of ethyl 4-(5-(3-((4-chloro-2-(4-ethoxy-4-oxobutanoyl)-6-methoxyisoindolin-5-yl)oxy)propoxy)-4-fluoro-6-methoxybenzo[b]thiophen-2-yl)-4-oxobutanoate (0.027 g, 38.8958 mol) in THF (1 mL) and Water (1 mL) at 20° C. The reaction mixture was stirred for 1 h at 20° C. The reaction mixture was adjusted to pH=3 with HCl (1 mol/L), The reaction mixture was purified on C18 column ACN/H₂O (0.1% FA)(0-52%). The pure fraction was concentrated and dried by lyophilization. There was 4-(5-(3-((2-(3-carboxypropanoyl)-4-chloro-6-methoxyisoindolin-5-yl)oxy)propoxy)-4-fluoro-6-methoxybenzo[b]thiophen-2-yl)-4-oxobutanoic acid (0.017 g, 26.6434 mol, 69.6846% yield) obtained as a white solid. LCMS: (ESI, m/z): [M+H]⁺=638.150. ¹H NMR (400 MHz, DMSO-d₆) δ 12.2(s, 2H), 8.29 (s, 1H), 7.56 (s, 1H), 7.07 (s, 0.5H), 7.03 (s, 0.5H), 4.83 (s, 1H), 4.72 (s, 1H), 4.62 (s, 1H), 4.49 (s, 1H), 4.32-4.25 (m, 2H), 4.17 (t, J=6.1 Hz, 2H), 3.89 (s, 3H), 3.80 (d, J=1.7 Hz, 3H), 3.30-3.27 (m, 2H), 2.61-2.55 (m, 4H), 2.48-2.46 (m, 2H), 2.09-2.06 (m, 2H).

### Example II-3

### 4-(5-(3-((2-(3-carboxypropanoyl)-4-chloro-6-methoxybenzo[b]thiophen-5-yl)oxy)propoxy)-4-fluoro-6-methoxyisoindolin-2-yl)-4-oxobutanoic acid

### Step a: tert-butyl 5-(3-bromopropoxy)-4-fluoro-6-methoxy-isoindoline-2-carboxylate

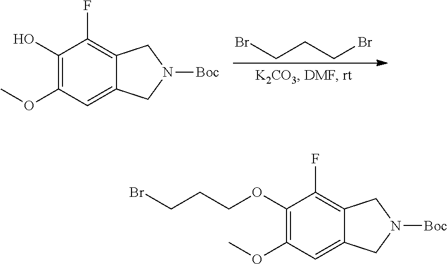 [SMILES: COc1cc2c(c(F)c1O)CN(C(=O)OC(C)(C)C)C2 ; BrCCCBr ; COc1cc2c(c(F)c1OCCCBr)CN(C(=O)OC(C)(C)C)C2]

To a solution of tert-butyl 4-fluoro-5-hydroxy-6-methoxy-isoindoline-2-carboxylate (0.50 g, 1.7649 mmol) in DMF (10 mL), was added 1,3-Dibromopropane (2.48 g, 12.2841 mmol) and K₂CO₃ (0.71 g, 5.1373 mmol). The reaction mixture was stirred for 3 h at 20° C. The reaction mixture was diluted with Ethyl acetate (200 mL), and washed sequentially with water (2×100 mL) and saturated brine (100 mL). The organic layer was dried over Na₂SO₄, filtered and evaporated to afford crude product. The crude product was purified on flash silica chromatography, elution gradient 0% to 100% Ethyl acetate in heptane. There was tert-butyl 5-(3-bromopropoxy)-4-fluoro-6-methoxy-isoindoline-2-carboxylate (0.637 g, 1.5757 mmol, 89.2763% yield) obtained as a white solid.

LCMS: (ESI, m/z): [M+H]⁺=331.100.

### Step b: tert-butyl 5-(3-((4-chloro-2-(4-ethoxy-4-oxobutanoyl)-6-methoxybenzo[b]thiophen-5-yl)oxy)propoxy)-4-fluoro-6-methoxyisoindoline-2-carboxylate

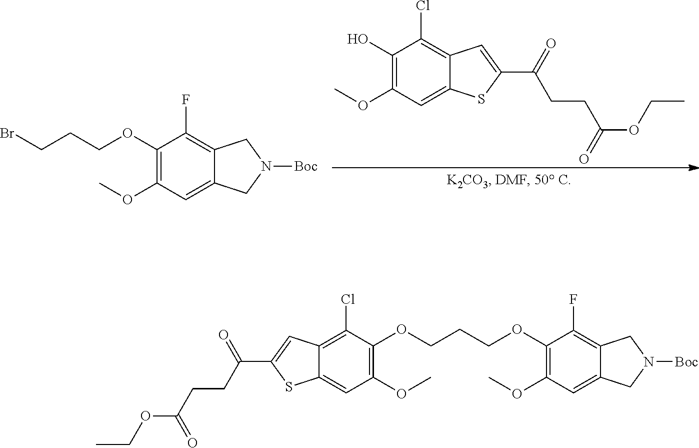 [SMILES: COc1cc2c(c(F)c1OCCCBr)CN(C(=O)OC(C)(C)C)C2 ; CCOC(=O)CCC(=O)c1cc2c(Cl)c(O)c(OC)cc2s1 ; CCOC(=O)CCC(=O)c1cc2c(Cl)c(OCCCOc3c(OC)cc4c(c3F)CN(C(=O)OC(C)(C)C)C4)c(OC)cc2s1]

K₂CO₃ (0.166 g, 1.2011 mmol) was added to a solution of ethyl 4-(4-chloro-5-hydroxy-6-methoxybenzo[b]thiophen-2-yl)-4-oxobutanoate (0.147 g, 428.8290 mol) and tert-butyl 5-(3-bromopropoxy)-4-fluoro-6-methoxy-isoindoline-2-carboxylate (0.173 g, 427.9318 mol) in DMF (5 mL) at 20° C. under N₂ atmosphere. The reaction mixture was stirred for 3 h at 50° C. The reaction mixture was purified on C18 column ACN/H₂O (0.1% FA)(0-75%). The pure fraction was concentrated and dried under vacuo. There was tert-butyl 5-(3-((4-chloro-2-(4-ethoxy-4-oxobutanoyl)-6-methoxybenzo[b]thiophen-5-yl)oxy)propoxy)-4-fluoro-6-methoxyisoindoline-2-carboxylate (0.138 g, 207.1598 mol, 48.3083% yield) obtained as a yellow solid. LCMS: (ESI, m/z): [M+H]⁺=666.190. ¹H NMR (400 MHz, DMSO-d₆) δ 8.21 (s, 1H), 7.74 (s, 1H), 6.89 (s, 0.5H), 6.87 (s, 0.5H), 4.53 (s, 4H), 4.21 (d, J=6.2, 4H), 4.06 (q, J=7.1 Hz, 2H), 3.90 (s, 3H), 3.77 (d, J=3.7 Hz, 3H), 3.39 (t, J=6.4 Hz, 2H), 2.66 (t, J=6.3 Hz, 2H), 2.11-2.09 (m, 2H), 1.45 (s, 9H), 1.18 (t, J=7.1 Hz, 3H).

### Step c: ethyl 4-(4-chloro-5-(3-((4-fluoro-6-methoxyisoindolin-5-yl)oxy)propoxy)-6-methoxybenzo[b]thiophen-2-yl)-4-oxobutanoate

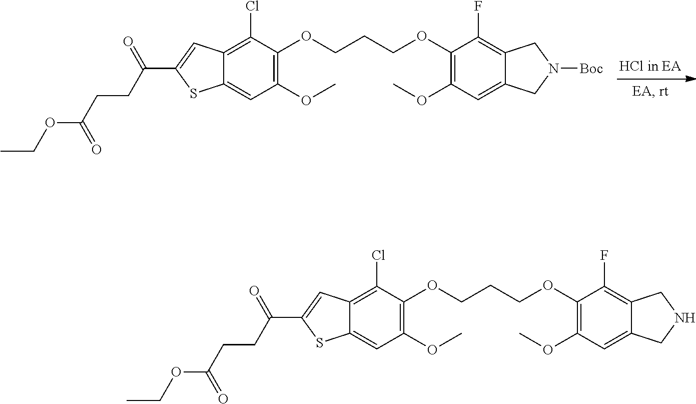 [SMILES: CCOC(=O)CCC(=O)c1cc2c(Cl)c(OCCCOc3c(OC)cc4c(c3F)CN(C(=O)OC(C)(C)C)C4)c(OC)cc2s1 ; CCOC(=O)CCC(=O)c1cc2c(Cl)c(OCCCOc3c(OC)cc4c(c3F)CNC4)c(OC)cc2s1]

HCl (5 mL, 20 mmol, 4 M in EA) was added to a solution of tert-butyl 5-(3-((4-chloro-2-(4-ethoxy-4-oxobutanoyl)-6-methoxybenzo[b]thiophen-5-yl)oxy)propoxy)-4-fluoro-6-methoxyisoindoline-2-carboxylate (0.117 g, 175.6356 mol) in EA (5 mL) at 20° C. The reaction mixture was stirred for 2 h at 20° C. The reaction mixture was evaporated under reduced pressure. There was ethyl 4-(4-chloro-5-(3-((4-fluoro-6-methoxyisoindolin-5-yl)oxy)propoxy)-6-methoxybenzo[b]thiophen-2-yl)-4-oxobutanoate (0.182 g, 321.5338 μmol, 183.0687% yield) obtained as a brown solid. LCMS: (ESI, m/z): [M+H]⁺=566.130. ¹H NMR (400 MHz, DMSO-d₆) δ 10.34 (s, 2H), 8.22 (s, 1H), 7.75 (s, 1H), 6.97 (s, 1H), 4.49 (s, 2H), 4.46 (s, 2H), 4.23-4.20 (m, 4H), 4.06 (q, J=7.1 Hz, 2H), 3.90 (s, 3H), 3.79 (s, 3H), 2.67 (t, J=6.3 Hz, 2H), 2.09-2.11 (m, 2H), 1.18 (t, J=7.1 Hz, 3H).

### Step d: ethyl 4-(5-(3-((4-chloro-2-(4-ethoxy-4-oxobutanoyl)-6-methoxybenzo[b]thiophen-5-yl)oxy)propoxy)-4-fluoro-6-methoxyisoindolin-2-yl)-4-oxobutanoate

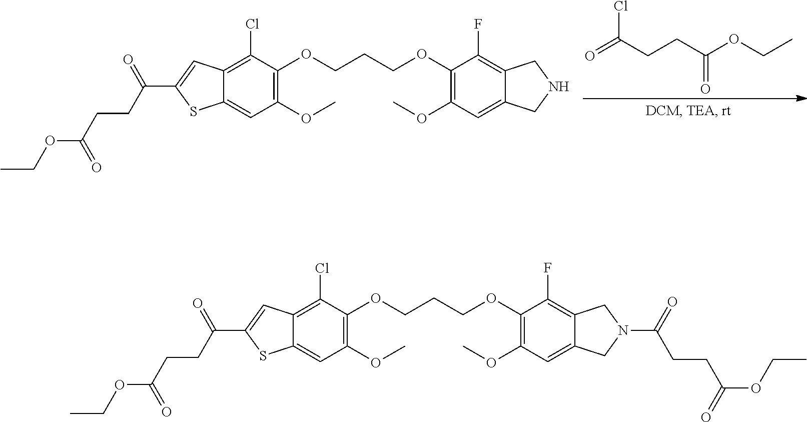

Ethyl Succinyl Chloride (0.097 g, 589.3557 mol) was added to a solution of ethyl 4-(4-chloro-5-(3-((4-fluoro-6-methoxyisoindolin-5-yl)oxy)propoxy)-6-methoxybenzo[b]thiophen-2-yl)-4-oxobutanoate (0.167 g, 295.0337 mol) and TEA (0.164 g, 1.6207 mmol) in DCM (5 mL) at 20° C. under N₂ atmosphere. The reaction mixture was stirred for 1 h at 20° C. The reaction mixture was evaporated under reduced pressure. The residue was purified on C18 column ACN/H₂O (0.1% FA)(0-60%). The pure fraction was concentrated and dried by lyophilization. There was ethyl 4-(5-(3-((4-chloro-2-(4-ethoxy-4-oxobutanoyl)-6-methoxybenzo[b]thiophen-5-yl)oxy)propoxy)-4-fluoro-6-methoxyisoindolin-2-yl)-4-oxobutanoate (0.081 g, 116.6874 mol, 39.5505% yield) obtained as a white solid. LCMS: (ESI, m/z): [M+H]⁺=694.180. ¹H NMR (400 MHz, DMSO-d₆) δ 8.22 (s, 1H), 7.74 (d, J=2.2 Hz, 1H), 6.91 (s, 0.5H), 6.89 (s, 0.5H), 4.81 (s, 1H), 4.80 (s, 1H), 4.57 (s, 2H), 4.22 (q, J=5.9 Hz, 4H), 4.09-4.03 (m, 4H), 3.90 (d, J=1.1 Hz, 3H), 3.78 (d, J=2.5 Hz, 3H), 3.40 (s, 2H), 2.70-2.58 (m, 4H), 2.57-2.53 (m, 2H), 2.15-2.04 (m, 2H), 1.26-1.14 (m, 6H).

### Step e: 4-(5-(3-((2-(3-carboxypropanoyl)-4-chloro-6-methoxybenzo[b]thiophen-5-yl)oxy)propoxy)-4-fluoro-6-methoxyisoindolin-2-yl)-4-oxobutanoic acid

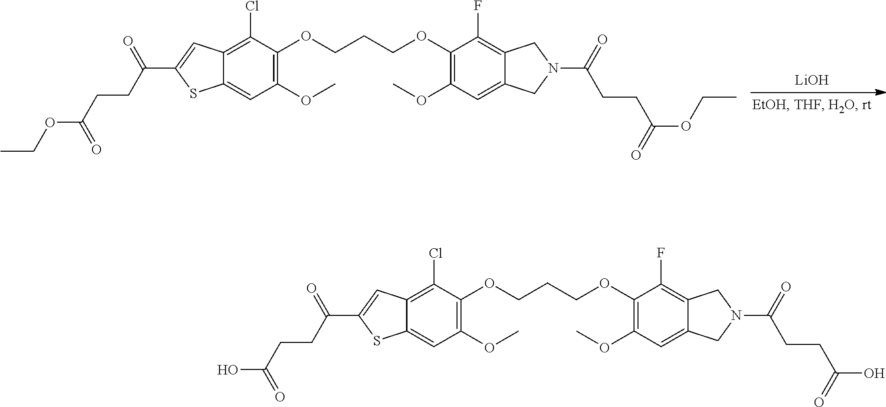 [SMILES: CCOC(=O)CCC(=O)c1cc2c(Cl)c(OCCCOc3c(OC)cc4c(c3F)CN(C(=O)CCC(=O)OCC)C4)c(OC)cc2s1 ; "LiOH" ; COc1cc2c(c(F)c1OCCCOc1c(OC)cc3sc(C(=O)CCC(=O)O)cc3c1Cl)CN(C(=O)CCC(=O)O)C2]

LiOH (0.045 g, 1.8790 mmol) was added to a solution of ethyl 4-(5-(3-((4-chloro-2-(4-ethoxy-4-oxobutanoyl)-6-methoxybenzo[b]thiophen-5-yl)oxy)propoxy)-4-fluoro-6-methoxyisoindolin-2-yl)-4-oxobutanoate (0.044 g, 63.3858 mol) in THF (2 mL), H₂O (2 mL) and Ethanol (1 mL) at 20° C. The reaction mixture was stirred for 1 h at 20° C. The reaction mixture was adjusted pH=3 with HCl (1 mol/L). The reaction mixture was evaporated under reduced pressure. The residue was purified on C18 column ACN/H₂O (0.1% FA)(0-52%). The pure fraction was concentrated and dried by lyophilization. There was 4-(5-(3-((2-(3-carboxypropanoyl)-4-chloro-6-methoxybenzo[b]thiophen-5-yl)oxy)propoxy)-4-fluoro-6-methoxyisoindolin-2-yl)-4-oxobutanoic acid (0.032 g, 50.1523 mol, 79.1223% yield) obtained as a white solid. LCMS: (ESI, m/z): [M+H]⁺=638.120. ¹H NMR (400 MHz, DMSO-d₆) δ 8.19 (s, 1H), 7.73 (s, 1H), 6.91 (s, 0.5H), 6.88 (s, 0.5H), 4.79 (s, 2H), 4.57 (s, 2H), 4.25-4.19 (m, 4H), 3.90 (s, 3H), 3.78 (d, J=1.7 Hz, 3H), 3.33 (t, J=12.0 Hz, 2H), 2.63-2.54 (m, 4H), 2.49-2.45 (m, 2H), 2.15-2.04 (m, 2H).

### Example II-4

### 4-(5-(3-((2-(3-carboxypropanoyl)-4-fluoro-6-methoxybenzo[b]thiophen-5-yl)oxy)propoxy)-4-fluoro-6-methoxyisoindolin-2-yl)-4-oxobutanoic acid

### Step a: ethyl 4-(5-(3-bromopropoxy)-4-fluoro-6-methoxyisoindolin-2-yl)-4-oxobutanoate

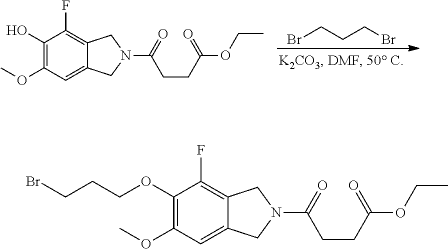

1,3-Dibromopropane (0.708 g, 3.5069 mmol) was added to a mixture of ethyl 4-(4-fluoro-5-hydroxy-6-methoxy-isoindolin-2-yl)-4-oxo-butanoate (0.217 g, 697.0656 mol) and Potassium carbonate (0.346 g, 2.5035 mmol) in DMF (5 mL) at 20° C. The reaction mixture was stirred for 3 h at 20° C. The reaction mixture was purified on C18 column ACN/H₂O (0.1% FA)(0-45%). The pure fraction was concentrated and dried under vacuo. There was ethyl 4-(5-(3-bromopropoxy)-4-fluoro-6-methoxyisoindolin-2-yl)-4-oxobutanoate (0.211 g, 488.1091 μmol, 70.0234% yield) obtained as a white solid. LCMS: (ESI, m/z): [M+H]⁺=432.070.

### Step b: ethyl 4-(5-(3-((2-(4-ethoxy-4-oxobutanoyl)-4-fluoro-6-methoxybenzo[b]thiophen-5-yl)oxy)propoxy)-4-fluoro-6-methoxyisoindolin-2-yl)-4-oxobutanoate

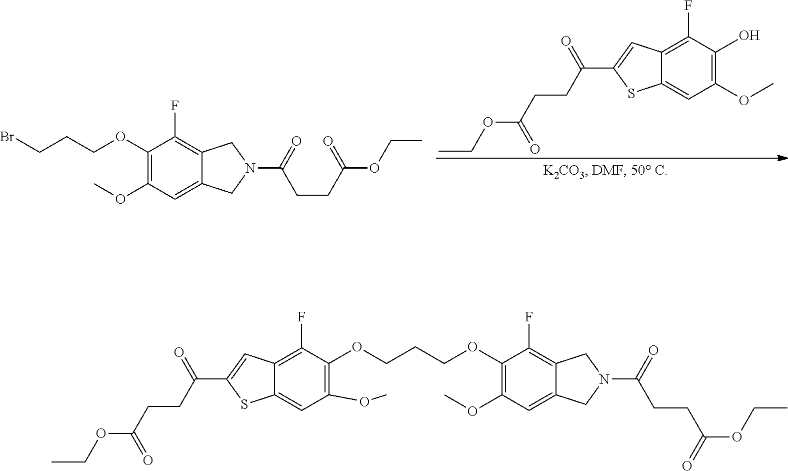

Potassium carbonate (0.079 g, 571.6126 mol) was added to a solution of ethyl 4-(5-(3-bromopropoxy)-4-fluoro-6-methoxyisoindolin-2-yl)-4-oxobutanoate (0.070 g, 161.9319 mol) and ethyl 4-(4-fluoro-5-hydroxy-6-methoxybenzo[b]thiophen-2-yl)-4-oxobutanoate (0.065 g, 199.1791 mol) in N,N-Dimethylformamide (3 mL) at 20° C. under N₂ atmosphere. The reaction mixture was stirred overnight at 50° C. The reaction mixture was purified on C18 column ACN/H₂O (0.1% FA)(0-70%). The pure fraction was concentrated and dried by lyophilization. There was ethyl 4-(5-(3-((2-(4-ethoxy-4-oxobutanoyl)-4-fluoro-6-methoxybenzo[b]thiophen-5-yl)oxy)propoxy)-4-fluoro-6-methoxyisoindolin-2-yl)-4-oxobutanoate (0.056 g, 82.6315 mol, 51.0285% yield) obtained as a yellow solid. LCMS: (ESI, m/z): [M+H]⁺=678.210. ¹H NMR (400 MHz, DMSO-d₆) δ 8.31 (s, 1H), 7.56 (s, 1H), 6.89 (s, 0.5H), 6.86 (s, 0.5H), 4.78 (s, 2H), 4.56 (s, 1H), 4.54 (s, 1H), 4.25 (t, J=6.0 Hz, 2H), 4.19 (t, J=6.0 Hz, 2H), 4.09-4.02 (m, 4H), 3.88 (s, 3H), 3.77 (d, J=2.0 Hz, 3H), 3.36 (t, J=6.4 Hz, 2H), 2.70-2.51 (m, 6H), 2.10-1.99 (m, 2H), 1.22-1.14 (m, 6H).

### Step c: 4-(5-(3-((2-(3-carboxypropanoyl)-4-fluoro-6-methoxybenzo[b]thiophen-5-yl)oxy)propoxy)-4-fluoro-6-methoxyisoindolin-2-yl)-4-oxobutanoic acid

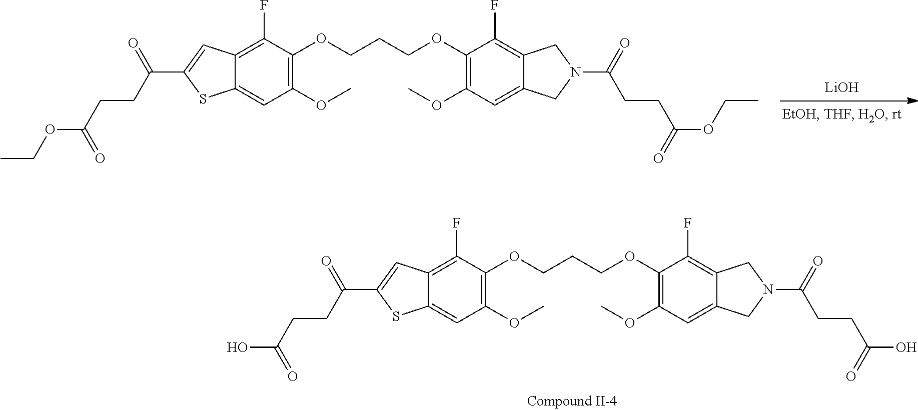 [SMILES: CCOC(=O)CCC(=O)c1cc2c(F)c(OCCCOc3c(OC)cc4c(c3F)CN(C(=O)CCC(=O)OCC)C4)c(OC)cc2s1 ; "LiOH" ; COc1cc2c(c(F)c1OCCCOc1c(OC)cc3sc(C(=O)CCC(=O)O)cc3c1F)CN(C(=O)CCC(=O)O)C2]

LiOH (0.043 g, 1.7955 mmol) was added to a solution of ethyl 4-(5-(3-((2-(4-ethoxy-4-oxobutanoyl)-4-fluoro-6-methoxybenzo[b]thiophen-5-yl)oxy)propoxy)-4-fluoro-6-methoxyisoindolin-2-yl)-4-oxobutanoate (0.039 g, 57.5469 mol) in THF (2 mL), H₂O (2 mL) and Ethanol (1 mL) at 20° C. The reaction mixture was stirred overnight at 20° C. The reaction mixture was adjusted to pH=3 with HCl (aq.1 M), The reaction mixture was evaporated under reduced pressure. The residue was purified on C18 column ACN/H₂O (0.1% FA)(0-50%). The pure fraction was concentrated and dried under vacuo. There was 4-(5-(3-((2-(3-carboxypropanoyl)-4-fluoro-6-methoxybenzo[b]thiophen-5-yl)oxy)propoxy)-4-fluoro-6-methoxyisoindolin-2-yl)-4-oxobutanoic acid (0.012 g, 19.3050 mol, 33.5465% yield) obtained as a white solid. LCMS: (ESI, m/z): [M+H]⁺=622.150. ¹H NMR (400 MHz, DMSO-d₆) δ 12.16 (s, 2H), 8.29 (s, 1H), 7.56 (s, 1H), 6.90 (s, 0.5H), 6.86 (s, 0.5H), 4.77 (s, 2H), 4.56 (s, 1H), 4.55 (s, 1H), 4.29-4.15 (m, 4H), 3.88 (s, 3H), 3.77 (s, 3H), 3.31-3.29 (m, 2H), 2.63-2.53 (m, 4H), 2.50-2.46 (m, 2H), 2.10-1.99 (m, 2H).

### Example II-5

### sodium (S)-4-(5-(3-((2-((S)-3-carboxylatobutanoyl)-6-methoxybenzo[b]thiophen-5-yl)oxy)propoxy)-6-methoxyisoindolin-2-yl)-2-methyl-4-oxobutanoate

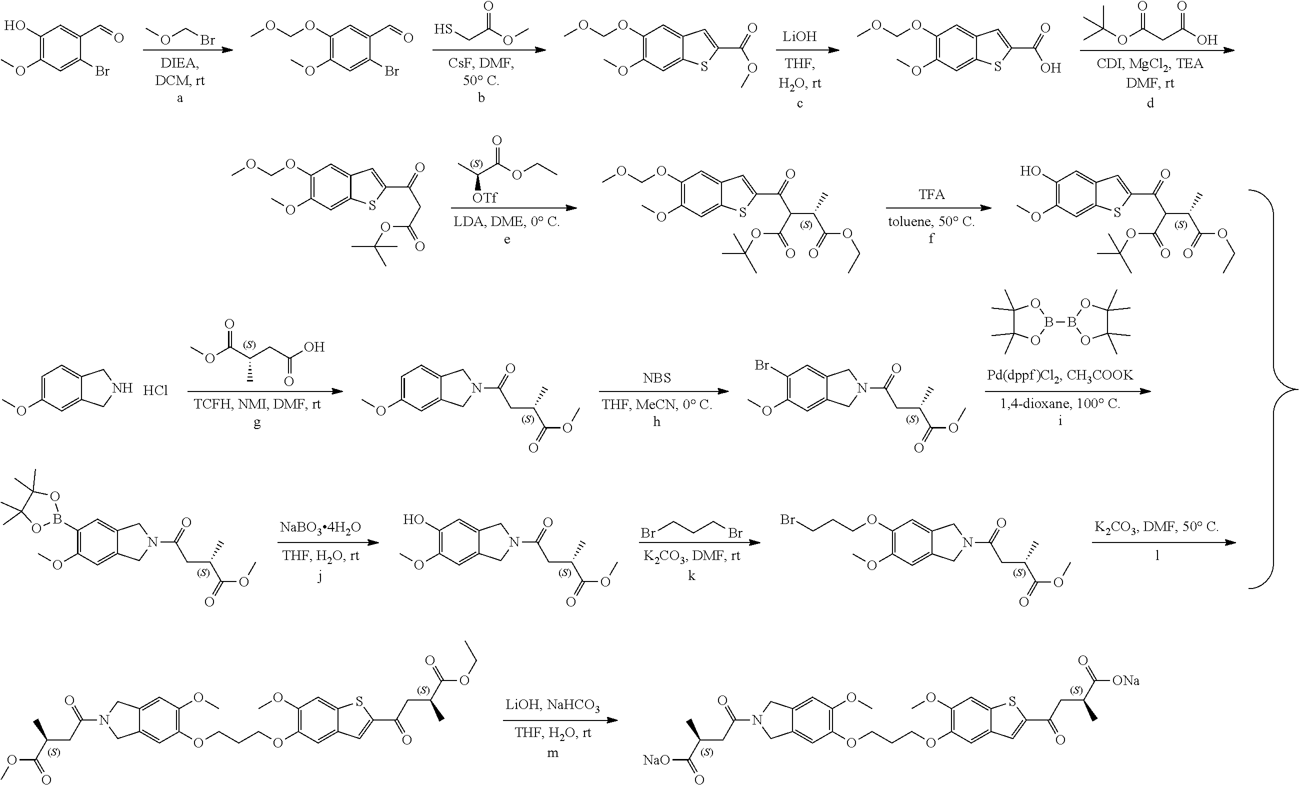

Step a: Bromomethyl methyl ether (60.56 g, 484.6196 mmol) was added to a solution of N,N-Diisopropylethylamine (97.64 g, 755.4796 mmol) and 2-bromo-5-hydroxy-4-methoxybenzaldehyde (102.80 g, 444.9386 mmol) in DCM (1500 mL) at 0° C. under N₂ atmosphere. The reaction mixture was stirred at 25° C. for 2 h. The resulting solution was quenched with adding of water (1000 mL) at 25° C. The resulting mixture was extracted with DCM (2×1000 mL), washed with water (500 mL) and brine (500 mL). The organics dried over Na₂SO₄, filtered and evaporated under reduced pressure. There was 2-bromo-4-methoxy-5-(methoxymethoxy)benzaldehyde (115.25 g, 418.9454 mmol, 94.1580% yield) obtained as a light yellow solid.

LCMS: (ESI, m/z): [M+H]⁺=274.984. ¹H NMR (400 MHz, DMSO-d₆) δ 10.06 (s, 1H), 7.51 (s, 1H), 7.39 (s, 1H), 5.25 (s, 2H), 3.93 (s, 3H), 3.39 (s, 3H).

Step b: Methyl thioglycolate (53.415 g, 503.2348 mmol) was added to a solution of 2-bromo-4-methoxy-5-(methoxymethoxy)benzaldehyde (115.11 g, 418.4365 mmol) and Caesium fluoride (130.32 g, 857.9136 mmol) in N,N-Dimethylformamide (1200 mL) at 25° C. under N₂ atmosphere. The reaction mixture was heated to 50° C. and stirred overnight. The reaction mixture was pour into the water (10 L) and stirred for 1 h. The precipitate was collected by filtration, washed with water (3×200 mL). The filter cake was dried under vacuo at 60° C. to obtain a crude product. The crude product was diluted with DCM (200 mL). Pour the mixture into n-Hexane (2000 mL) and stirred for 1 h. The precipitate was collected by filtration, washed with n-Hexane (3×100 mL). The filter cake was dried under vacuo at 60° C. There was methyl 6-methoxy-5-(methoxymethoxy)benzothiophene-2-carboxylate (65.50 g, 232.0131 mmol, 55.4476% yield) obtained as a yellow solid. LCMS: (ESI, m/z): [M+H]⁺=284.056. ¹H NMR (400 MHz, DMSO-d₆) δ 8.07 (s, 1H), 7.69 (s, 1H), 7.65 (s, 1H), 5.25 (s, 2H), 3.90 (s, 3H), 3.88 (s, 3H), 3.44 (s, 3H).

Step c: LiOH (17.58 g, 734.0813 mmol) was added to a solution of methyl 6-methoxy-5-(methoxymethoxy)benzothiophene-2-carboxylate (51.16 g, 181.2181 mmol) in THF (600 mL) and Water (200 mL) at 25° C. The reaction mixture was stirred for 4 h at 25° C. The reaction mixture was evaporated under reduced pressure. The residue was diluded with water and adjusted to pH=4 with HCl (1 M). The precipitate was collected by filtration, washed with water (2×50 mL). The filtrate cake was dried under vacuo. There was 6-methoxy-5-(methoxymethoxy)benzothiophene-2-carboxylic acid (44.17 g, 164.6382 mmol, 90.8509% yield) obtained as a yellow solid. LCMS: (ESI, m/z): [M−H]—=267.041. ¹H NMR (400 MHz, DMSO-d₆) δ 13.17 (s, 1H), 7.95 (s, 1H), 7.64 (s, 1H), 7.62 (s, 1H), 5.23 (s, 2H), 3.88 (s, 3H), 3.43 (s, 3H).

Step d: To a stirred solution of 6-methoxy-5-(methoxymethoxy)benzothiophene-2-carboxylic acid (53.21 g, 198.3337 mmol) in DMF (1000 mL) was added CDI (64.55 g, 398.0919 mmol) at 25° C. The reaction mixture was stirred for 2 h at 25° C. To the mixture above was added 3-(tert-butoxy)-3-oxopropanoic acid (49.03 g, 306.1174 mmol), TEA (62.33 g, 615.9736 mmol) and Magnesium chloride (29.11 g, 305.7420 mmol). The reaction mixture was stirred overnight at 25° C. The reaction mixture was concentrated and diluted with EA (3000 mL), washed with water (2×3000 mL) and brine (3000 mL). The organics dried over Na₂SO₄, filtered and evaporated under reduced pressure. The residue was purified on silica gel column EA/n-Hexane (0-50%). The pure fraction was concentrated and dried under vacuo. There was tert-butyl 3-(6-methoxy-5-(methoxymethoxy)benzo[b]thiophen-2-yl)-3-oxopropanoate (36.19 g, 98.7643 mmol, 49.7971% yield) obtained as a yellow oil. LCMS: (ESI, m/z): [M+H]⁺=367.114. ¹H NMR (400 MHz, DMSO-d₆) δ 8.20 (s, 1H), 7.67 (s, 1H), 7.62 (s, 1H), 5.24 (s, 2H), 4.05 (s, 2H), 3.89 (s, 3H), 3.42 (s, 3H), 1.41 (s, 9H).

Step e: LDA (7.9020 g, 73.7662 mmol) was added to a solution of tert-butyl 3-(6-methoxy-5-(methoxymethoxy)benzo[b]thiophen-2-yl)-3-oxopropanoate (18.02 g, 49.1775 mmol) in 2,5-Dioxahexane (200 mL) at 0° C. under N₂ atmosphere. The reaction mixture was stirred for 40 min at 0° C. under N₂ atmosphere. Ethyl (S)-2-(((trifluoromethyl)sulfonyl)oxy)propanoate (18.06 g, 72.1843 mmol) was added to the mixture at 0° C. under N₂ atmosphere. The reaction mixture was stirred for 3 h at 0° C. under N₂ atmosphere. The reaction mixture was quenched with adding of KHCO₃ (100 mL) at 0° C. The reaction mixture was concentrated and diluted with EA (500 mL), washed with water (200 mL) and brine (100 mL). The organics dried over Na₂SO₄, filtered and evaporated under reduced pressure. The residue was purified on silica gel column EA/n-Hexane (0-100%). The pure fraction was concentrated and dried under vacuo. There was 1-(tert-butyl) 4-ethyl(3S)-2-(6-methoxy-5-(methoxymethoxy)benzo[b]thiophene-2- carbonyl)-3-methylsuccinate (21.55 g, 46.1908 mmol, 93.9267% yield) obtained as a yellow oil. LCMS: (ESI, m/z): [M+H]⁺=467.166.

Step f: TFA (101.31 g, 888.5026 mmol) was added to a solution of 1-(tert-butyl) 4-ethyl(3S)-2-(6-methoxy-5-(methoxymethoxy)benzo[b]thiophene-2- carbonyl)-3-methylsuccinate (21.52 g, 46.1265 mmol) in Toluene (198 mL) at 25° C. The reaction mixture was heated to 50° C. and stirred for 2 h. The reaction mixture was evaporated under reduced pressure. The residue was purified on silica gel column EA/n-Hexane (0-50%). The pure fraction was concentrated and dried under vacuo. There was ethyl (2S)-4-(5-hydroxy-6-methoxy-benzothiophen-2-yl)-2-methyl-4-oxo-butanoate (6.78 g, 21.0314 mmol, 45.5950% yield) obtained as a yellow solid. LCMS: (ESI, m/z): [M+H]⁺=323.087. ¹H NMR (400 MHz, DMSO-d₆) δ 9.39 (s, 1H), 8.17 (s, 1H), 7.54 (s, 1H), 7.31 (s, 1H), 4.10-3.98 (m, 2H), 3.87 (s, 3H), 3.44-3.35 (m, 1H), 3.19-3.08 (m, 1H), 2.98-2.88 (m, 1H), 1.21-1.10 (m, 6H).

Step g: NMI (7.96 g, 96.9507 mmol) was added to a solution of TCFH (8.11 g, 28.9045 mmol) and (S)-4-methoxy-3-methyl-4-oxobutanoic acid (2.81 g, 19.2280 mmol) in DMF (100 mL) at 25° C. The reaction mixture was stirred for 10 min at 20° C. 5-methoxyisoindoline hydrochloride (4.99 g, 26.8785 mmol) was added to the solution at 25° C. The reaction mixture was stirred 2 h at 25° C. The reaction mixture was concentrated and diluted with H₂O (200 mL), extracted with EA (2×200 mL) and washed with brine (100 mL). The organics dried over Na₂SO₄, filtered and evaporated under reduced pressure. The residue was purified on silica gel column EA/n-Hexane (0-55%). The pure fraction was concentrated and dried under vacuo. There was methyl (2S)-4-(5-methoxyisoindolin-2-yl)-2-methyl-4-oxo-butanoate (3.49 g, 12.5850 mmol, 65.4512% yield) obtained as a yellow solid. LCMS: (ESI, m/z): [M+H]⁺=278.130. ¹H NMR (400 MHz, DMSO-d₆) δ 7.23 (t, J=8.0 Hz, 1H), 6.95-6.84 (m, 2H), 4.78 (s, 1H), 4.73 (s, 1H), 4.56 (s, 1H), 4.52 (s, 1H), 3.75 (d, J=1.8 Hz, 3H), 3.60 (s, 3H), 2.91-2.81 (m, 1H), 2.76-2.66 (m, 1H), 2.54-2.51 (m, 0.5H), 2.49-2.45 (m, 0.5H), 1.18-1.14 (m, 3H).

Step h: NBS (3.717 g, 20.8839 mmol) was added to a solution of methyl (2S)-4-(5-methoxyisoindolin-2-yl)-2-methyl-4-oxo-butanoate (3.426 g, 12.3542 mmol) in THF (30 mL) and MeCN (30 mL) at 0° C. The reaction mixture was stirred for 3 h at 25° C. The reaction mixture was concentrated and diluted with DCM (200 mL), washed with H₂O (100 mL), KHCO₃ (aq)(2×100 mL) and brine (100 mL). The organics dried over Na₂SO₄, filtered and evaporated under reduced pressure. The residue was purified on silica gel column EA/n-Hexane (0-60%). The pure fraction was concentrated and dried under vacuo. There was methyl (2S)-4-(5-bromo-6-methoxy-isoindolin-2-yl)-2-methyl-4-oxo-butanoate (3.02 g, 8.4781 mmol, 68.6255% yield) obtained as a yellow solid.

LCMS: (ESI, m/z): [M+H]⁺=356.040. ¹H NMR (400 MHz, DMSO-d₆) δ 7.57 (s, 0.5H), 7.55 (s, 0.5H), 7.14 (s, 0.5H), 7.09 (s, 0.5H), 4.78 (s, 1H), 4.75 (s, 1H), 4.56 (s, 1H), 4.53 (s, 1H), 3.86 (s, 3H), 3.59 (s, 3H), 2.93-2.79 (m, 1H), 2.78-2.64 (m, 1H), 2.49-2.46 (m, 1H), 1.15 (d, J=7.1 Hz, 3H).

Step i: Potassium Acetate (2.81 g, 28.6319 mmol) was added to a mixture of methyl (2S)-4-(5-bromo-6-methoxy-isoindolin-2-yl)-2-methyl-4-oxo-butanoate (3.00 g, 8.4220 mmol), [1,1′-Bis(diphenylphosphino)ferrocene]dichloropalladium(II) (0.74 g, 1.0113 mmol) and 4,4,4′,4′,5,5,5′,5′-Octamethyl-2,2′-bi(1,3,2-dioxaborolane) (3.18 g, 12.5228 mmol) in 1,4-Dioxane (100 mL) at 25° C. The reaction mixture was heated to 100° C. and stirred overnight. The reaction mixture was diluted with EA(200 mL), The precipitate was collected by filtration, washed with EA (1×100 mL). The filtrate was evaporated under reduced pressure. The residue was purified on silica gel column EA/n-Hexane (0-50%). The pure fraction was concentrated and dried under vacuo. There was methyl (2S)-4-[5-methoxy-6-(4,4,5,5-tetramethyl-1,3,2-dioxaborolan-2-yl)isoindolin-2-yl]-2-methyl-4-oxo-butanoate (2.38 g, 5.9017 mmol, 70.0746% yield) obtained as a brown solid. LCMS: (ESI, m/z): [M+H]⁺=404.222. ¹H NMR (400 MHz, DMSO-d₆) δ 7.49 (s, 0.5H), 7.46 (s, 0.5H), 6.97 (s, 0.5H), 6.93 (s, 0.5H), 4.81 (s, 1H), 4.73 (s, 1H), 4.59 (s, 1H), 4.52 (s, 1H), 3.73 (s, 3H), 3.59 (s, 3H), 2.90-2.81 (m, 1H), 2.76-2.65 (m, 1H), 2.54-2.51 (m, 1H), 1.27 (s, 12H), 1.15 (d, J=6.6 Hz, 3H).

Step j: Sodium perborate tetrahydrate (0.80 g, 5.1995 mmol) was added to a solution of methyl (2S)-4-[5-methoxy-6-(4,4,5,5-tetramethyl-1,3,2-dioxaborolan-2-yl)isoindolin-2-yl]-2-methyl-4-oxo-butanoate (1.99 g, 4.9346 mmol) in THF (25 mL) and H₂O (25 mL) at 25° C. The reaction mixture was stirred for 1 h at 25° C. The reaction mixture was concentrated and diluted with H₂O(200 mL), extracted with DCM (3×100 mL) and washed with brine (100 mL). The organics dried over Na₂SO₄, filtered and evaporated under reduced pressure. The residue was purified on C18 column ACN/H₂O (0-40%). The pure fraction was concentrated and dried under vacuo. There was methyl (2S)-4-(5-hydroxy-6-methoxy-isoindolin-2-yl)-2-methyl-4-oxo-butanoate (1.23 g, 4.1935 mmol, 84.9809% yield) obtained as a white solid. LCMS: (ESI, m/z): [M+H]⁺=294.130.

Step k: Potassium carbonate (0.462 g, 3.3428 mmol) was added to a solution of methyl (2S)-4-(5-hydroxy-6-methoxy-isoindolin-2-yl)-2-methyl-4-oxo-butanoate (0.311 g, 1.0603 mmol) and 1,3-Dibromopropane (1.158 g, 5.7359 mmol) in DMF (20 mL) at 25° C. The reaction mixture was stirred for 3 h at 25° C. The reaction mixture was purified on C18 column ACN/H₂O (0-45%). The pure fraction was concentrated and dried under vacuo. There was methyl (2R)-4-[5-(3-bromopropoxy)-6-methoxy-isoindolin-2-yl]-2-methyl-4-oxo-butanoate (0.314 g, 757.9234 mol, 71.4822% yield) obtained as a colorless oil. LCMS: (ESI, m/z): [M+H]⁺=414.080.

Step l: Potassium carbonate (0.214 g, 1.5484 mmol) was added to a solution of methyl (S)-4-(5-(3-bromopropoxy)-6-methoxyisoindolin-2-yl)-2-methyl-4-oxobutanoate (0.206 g, 497.2362 mol) and ethyl (2S)-4-(5-hydroxy-6-methoxy-benzothiophen-2-yl)-2-methyl-4-oxo-butanoate (0.243 g, 753.7805 mol) in DMF (5 mL) at 25° C. The reaction mixture was heated to 50° C. and stirred overnight. The reaction mixture was concentrated and diluted with EA (200 mL), washed with water (100 mL) and brine (100 mL). The organics dried over Na₂SO₄, filtered and evaporated under reduced pressure. The residue was purified on silica gel column EA/n-Hexane (0-100%). The pure fraction was concentrated and dried under vacuo. There was ethyl (S)-4-(6-methoxy-5-(3-((6-methoxy-2-((S)-4-methoxy-3-methyl-4-oxobutanoyl)isoindolin-5-yl)oxy)propoxy)benzo[b]thiophen-2-yl)-2-methyl-4-oxobutanoate (0.130 g, 198.2454 mol, 39.8695% yield) obtained as a yellow solid. LCMS: (ESI, m/z): [M+H]⁺=656.245.

Step m: LiOH (0.017 g, 709.8625 mol) was added to a solution of ethyl (S)-4-(6-methoxy-5-(3-((6-methoxy-2-((S)-4-methoxy-3-methyl-4-oxobutanoyl)isoindolin-5-yl)oxy)propoxy)benzo [b]thiophen-2-yl)-2-methyl-4-oxobutanoate (0.120 g, 182.9957 mol) in THF (4 mL) and Water (1 mL) at 25° C. The reaction mixture was stirred overnight at 25° C. The reaction mixture was adjusted to pH=7 with HCl (1 M). The reaction mixture was evaporated under reduced pressure to obtain a crude product. The crude product was purified by Prep-HPLC with the following conditions: (CXTH LC6000, HPLC-P1): Column, Agela Durashell C18, 30 mm*250 mm, 10 μm; mobile phase, Water (0.1% NH₃.H₂O) and MeCN-(5-20-20% B(2-32-60 min)); Detector, uv 210 nm). The solvent was removed by lyophilization. NaHCO₃ (0.019 g, 226.1727 mol) was added to the lyophilized product in water, and stirred for 30 min at 25° C. The solvent was removed by lyophilization. There was sodium (S)-4-(5-(3-((2-((S)-3-carboxylatobutanoyl)-6-methoxybenzo [b]thiophen-5-yl)oxy)propoxy)-6-methoxyisoindolin-2-yl)-2-methyl-4-oxobutanoate (0.064 g, 97.3180 mol, 53.1805% yield) obtained as a white solid. LCMS: (ESI, m/z): [M+H]⁺=614.198. ¹H NMR (400 MHz, DMSO-d₆) δ 8.08 (s, 0.5H), 8.05 (s, 0.5H), 7.56 (s, 1H), 7.51 (s, 0.5H), 7.50 (s, 0.5H), 7.02-6.89 (m, 2H), 4.90-4.77 (m, 1H), 4.73-4.62 (m, 1H), 4.50 (s, 2H), 4.24-4.16 (m, 2H), 4.15-4.05 (m, 2H), 3.85 (s, 3H), 3.74 (s, 3H), 3.45-3.38 (m, 2H), 2.75-2.57 (m, 2H), 2.47-2.39 (m, 1H), 2.27-2.14 (m, 2H), 2.11-1.97 (m, 1H), 1.02 (t, J=6.5 Hz, 6H).

### Example II-6

### sodium (S)-4-(5-(3-((2-((S)-3-carboxylatobutanoyl)-4-fluoro-6-methoxybenzo[b]thiophen-5-yl)oxy)propoxy)-6-methoxyisoindolin-2-yl)-2-methyl-4-oxobutanoate

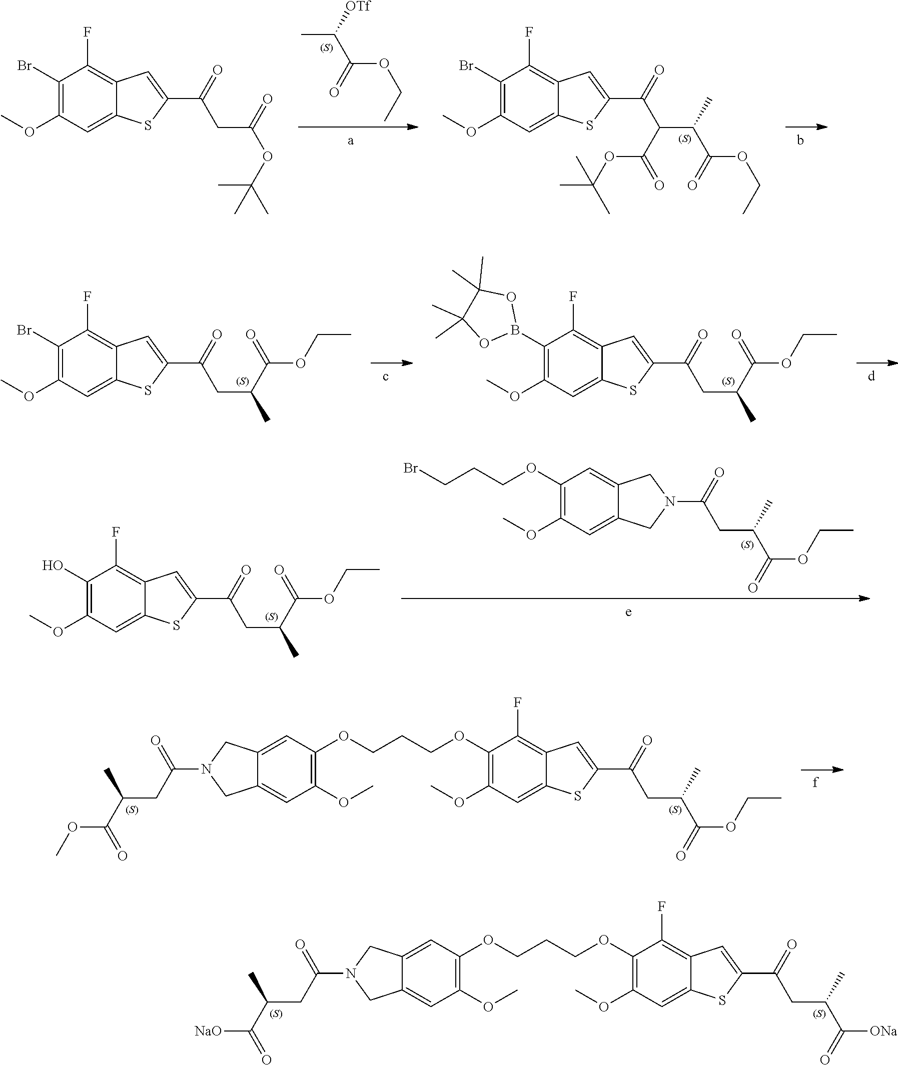

Step a: Lithium diisopropylamide, (552.5297 mg, 5.1579 mmol) was added to a solution of tert-butyl 3-(5-bromo-4-fluoro-6-methoxybenzo[b]thiophen-2-yl)-3-oxopropanoate (1.6 g, 3.9676 mmol) in 2,5-Dioxahexane (50 mL) at 0° C. under N₂ atmosphere. The reaction mixture was stirred for 40 min at 0° C. under N₂ atmosphere. Ethyl (S)-2-(((trifluoromethyl)sulfonyl)oxy)propanoate (2.9780 g, 11.9029 mmol) was added to the mixture at 0° C. under N₂ atmosphere. The reaction mixture was stirred overnight at 25° C. under N₂ atmosphere. The reaction mixture was concentrated and diluted with EA (100 mL), washed with NaHCO₃ (aq) (2×500 mL) and brine (50 mL). The organics dried over Na₂SO₄, filtered and evaporated under reduced pressure. The residue was purified on silica gel column EA/n-Hexane (0-20%). The pure fraction was concentrated and dried under vacuo. There was 1-(tert-butyl) 4-ethyl (3S)-2-(5-bromo-4-fluoro-6-methoxybenzo[b]thiophene-2-carbonyl)-3-methylsuccinate (1.15 g, 2.2846 mmol, 57.5800% yield) obtained as a colorless oil. LCMS: (ESI, m/z): [M+H]⁺=503.046. ¹H NMR (400 MHz, DMSO-d₆) δ 8.61 (s, 0.5H), 8.46 (s, 0.5H), 7.73 (s, 1H), 4.93-4.77 (m, 1H), 4.13-4.02 (m, 2H), 4.00-3.94 (m, 3H), 3.24-3.11 (m, 1H), 1.31 (d, J=11.5 Hz, 9H), 1.25-1.16 (m, 3H), 1.10 (t, J=7.8 Hz, 3H).

Step b: Trifluoroacetic acid (4 mL) was added to a solution of 1-(tert-butyl) 4-ethyl (3S)-2-(5-bromo-4-fluoro-6-methoxybenzo[b]thiophene-2-carbonyl)-3-methylsuccinate (1.02 g, 2.0263 mmol) in Toluene (20 mL) at 25° C.

The reaction mixture was stirred at 50° C. for 2 h. The reaction mixture was concentrated and purified on C-18 column MeCN/water (0-100%). The pure fraction was concentrated and dried under vacuo. There was ethyl (S)-4-(5-bromo-4-fluoro-6-methoxybenzo[b]thiophen-2-yl)-2-methyl-4-oxobutanoate (0.76 g, 1.8846 mmol, 93.0079% yield) obtained as a yellow solid. LCMS: (ESI, m/z): [M+H]⁺=402.994. ¹H NMR (400 MHz, MeOD-d₄) δ 8.14 (s, 1H), 7.40 (s, 1H), 4.19-4.03 (m, 2H), 3.98 (s, 3H), 3.60-3.39 (m, 1H), 3.25-2.97 (m, 2H), 1.28 (d, J=7.1 Hz, 3H), 1.23 (t, J=7.1 Hz, 3H).

Step c: [1,1′-Bis(diphenylphosphino)ferrocene]dichloropalladium(II) (0.13 g, 177.6676 mol) was added to a mixture of Potassium Acetate (0.53 g, 5.4003 mmol) ethyl (2S)-4-(5-bromo-4-fluoro-6-methoxy-benzothiophen-2-yl)-2-methyl-4-oxo-butanoate (0.70 g, 1.7358 mmol) and 4,4,4′,4′,5,5,5′,5′-Octamethyl-2,2′-bi(1,3,2-dioxaborolane) (0.87 g, 3.4260 mmol) in 1,4-Dioxane (30 mL) at 25° C. The reaction mixture was stirred overnight at 100° C. under N₂ atmosphere. The reaction mixture was evaporated under reduced pressure. The residue was concentrated and diluted with EA (200 mL), washed with NaHCO₃ (aq) (2×100 mL) and brine (100 mL). The organics dried over Na₂SO₄, filtered and evaporated under reduced pressure. The residue was purified on silica gel column EA/Hept (0-50%). The pure fraction was concentrated and dried under vacuo. There was ethyl (S)-4-(4-fluoro-6-methoxy-5-(4,4,5,5-tetramethyl-1,3,2-dioxaborolan-2-yl)benzo[b]thiophen-2-yl)-2-methyl-4-oxobutanoate (452 mg, 1.0037 mmol, 57.8229% yield) obtained as a white solid. LCMS: (ESI, m/z): [M+H]⁺=451.168. ¹H NMR (400 MHz, DMSO-d₆) δ 8.29 (s, 1H), 7.49 (s, 1H), 4.15-3.99 (m, 2H), 3.86 (s, 3H), 3.57-3.40 (m, 2H), 3.18 (s, 1H), 1.41-1.23 (m, 12H), 1.19-1.14 (m, 6H).

Step d: Sodium perborate tetrahydrate (0.23 g, 1.4949 mmol) was added to a solution of ethyl (S)-4-(4-fluoro-6-methoxy-5-(4,4,5,5-tetramethyl-1,3,2-dioxaborolan-2-yl)benzo[b]thiophen-2-yl)-2-methyl-4-oxobutanoate (0.46 g, 1.0215 mmol) in Tetrahydrofuran (20 mL) and Water (20 mL) at 25° C. The reaction mixture was stirred at 25° C. for 1 h. The reaction mixture was evaporated under reduced pressure. The residue was concentrated and diluted with DCM (200 mL), washed with water (2×100 mL) and brine (200 mL). The organics dried over Na₂SO₄, filtered and evaporated under reduced pressure. There was ethyl (2S)-4-(4-fluoro-5-hydroxy-6-methoxy-benzothiophen-2-yl)-2-methyl-4-oxo-butanoate (0.28 g, 822.6441 μmol, 80.5345% yield) obtained as a yellow solid. LCMS: (ESI, m/z): [M+H]⁺=341.078. ¹H NMR (400 MHz, DMSO-d₆) δ 9.51 (s, 1H), 8.25 (s, 1H), 7.46 (s, 1H), 4.09-4.00 (m, 2H), 3.96-3.88 (m, 3H), 3.52-3.41 (m, 1H), 3.24-3.16 (m, 1H), 2.94 (s, 1H), 1.22-1.10 (m, 6H).

Step e: Ethyl (2S)-4-(4-fluoro-5-hydroxy-6-methoxy-benzothiophen-2-yl)-2-methyl-4-oxo-butanoate (0.16 g, 470.0822 mol) was added to a mixture of methyl (S)-4-(5-(3-bromopropoxy)-6-methoxyisoindolin-2-yl)-2-methyl-4-oxobutanoate (0.20 g, 482.7536 mol) and Potassium carbonate (0.15 g, 1.0853 mmol) in N,N-Dimethylformamide (5 mL) at 25° C. The reaction mixture was stirred overnight at 25° C. The reaction mixtire was purified on C18 column ACN/H₂O (0-45%). The pure fraction was concentrated and dried under vacuo. There was ethyl (S)-4-(4-fluoro-6-methoxy-5-(3-((6-methoxy-2-((S)-4-methoxy-3-methyl-4-oxobutanoyl)isoindolin-5-yl)oxy)propoxy)benzo[b]thiophen-2-yl)-2-methyl-4-oxobutanoate (0.268 g, 397.7772 mol, 82.3976% yield) obtained as a colorless oil. LCMS: (ESI, m/z): [M+H]⁺=674.236. ¹H NMR (400 MHz, DMSO-d₆) δ 8.31 (s, 1H), 7.57 (s, 0.5H), 7.54 (s, 0.5H), 7.03-6.83 (m, 2H), 4.72 (s, 2H), 4.52 (s, 2H), 4.31-4.09 (m, 4H), 4.09-3.96 (m, 2H), 3.87 (s, 3H), 3.72 (s, 3H), 3.59 (s, 3H), 3.51-3.41 (m, 1H), 3.24-3.15 (m, 1H), 3.02-2.65 (m, 4H), 2.12 (s, 2H), 1.32-0.96 (m, 9H).

Step f: LiOH (0.030 g, 1.2527 mmol) was added to a solution of ethyl (2S)-4-[4-fluoro-6-methoxy-5-[3-[6-methoxy-2-[(3S)-4-methoxy-3-methyl-4-oxo-butanoyl]isoindolin-5-yl]oxypropoxy]benzothiophen-2-yl]-2-methyl-4-oxo-butanoate (0.258 g, 382.9348 mol) in Tetrahydrofuran (10 mL) and Water (2 mL) at 25° C. The reaction mixture was stirred overnight at 25° C. The reaction mixture was purified by HPLC. The pure fraction was concentrated and dried under vacuo to obtain the free acid. NaHCO₃ (0.016 g, 190.4612 mol) was added to the free acid in Acetonitrile (10 mL) and Water (10 mL) at 25° C. The mixture was stirred at 25° C. for 1 h. The solvent was removed by lyophilization. There was sodium (S)-4-(5-(3-((2-((S)-3-carboxylatobutanoyl)-4-fluoro-6-methoxybenzo[b]thiophen-5-yl)oxy)propoxy)-6-methoxyisoindolin-2-yl)-2-methyl-4-oxobutanoate (0.063 g, 93.2466 mol, 24.3505% yield) obtained as a white solid. LCMS: (ESI, m/z): [M+H]⁺=632.289. ¹H NMR (400 MHz, DMSO-d₆) δ 8.17-8.05 (m, 1H), 7.51 (s, 1H), 6.96-6.80 (m, 2H), 4.92-4.72 (m, 2H), 4.74-4.58 (m, 2H), 4.48 (s, 2H), 4.29-4.08 (m, 5H), 3.87 (s, 3H), 3.74-3.65 (m, 3H), 2.80-2.57 (m, 4H), 2.10 (s, 3H), 1.03 (d, J=6.9 Hz, 6H).

### Example II-7

### sodium 4-(5-(3-((2-(3-carboxylatopropanoyl)-6-hydroxybenzo[b]thiophen-5-yl)oxy)propoxy)-6-methoxyisoindolin-2-yl)-4-oxobutanoate

### Step a: 5-bromo-2-fluoro-4-(methoxymethoxy)benzaldehyde

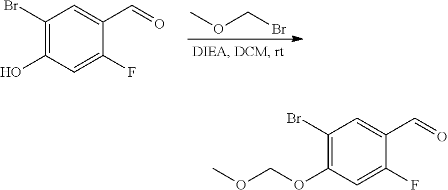

Bromomethyl methyl ether (0.71 g, 5.6816 mmol) was added to a solution of DIEA (0.83 g, 6.4220 mmol) and 5-bromo-2-fluoro-4-hydroxybenzaldehyde (0.94 g, 4.2921 mmol) in DCM (20 mL) at 0° C. The reaction mixture was stirred for 1 h at 25° C. The reaction mixture was quenched with adding of water (10 mL) at 20° C. The resulted mixture was washed with water (20 mL) and brine (20 mL). The organics dried over Na₂SO₄, filtered and evaporated under reduced pressure. There was a crude 5-bromo-2-fluoro-4-(methoxymethoxy)benzaldehyde (1.16 g, 4.4096 mmol, 102.7388% yield) obtained as a white solid. LCMS: (ESI, m/z): [M+H]⁺=262.950. ¹H NMR (400 MHz, DMSO-d₆) δ 10.03 (s, 1H), 8.02 (d, J=7.5 Hz, 1H), 7.28 (d, J=12.6 Hz, 1H), 5.45 (s, 2H), 3.43 (s, 3H).

### Step b: methyl 5-bromo-6-(methoxymethox y)benzothiophene-2-carboxylate

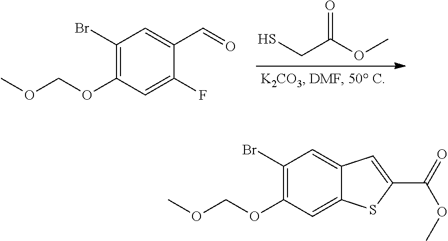

Methyl thioglycolate (0.4 mL, 4.4732 mmol) was added to a solution of 5-bromo-2-fluoro-4-(methoxymethoxy)benzaldehyde (1.06 g, 4.0295 mmol) and Potassium carbonate (0.15 g, 1.0853 mmol) in DMF (10 mL) at 25° C. The reaction mixture was heated to 50° C. and stirred for 2 h. The reaction mixture was quenched with adding of water (100 mL) at 20° C. The precipitate was collected by filtration, washed with water (3×20 mL).

The filtrate cake was dried under vacuo at 60° C. There was methyl 5-bromo-6-(methoxymethox y)benzothiophene-2-carboxylate (1.12 g, 3.3818 mmol, 83.9267% yield) obtained as a white solid. LCMS: (ESI, m/z): [M+H]⁺=331.100.

### Step c: 5-bromo-6-(methoxymethoxy) benzothiophene-2-carboxylic acid

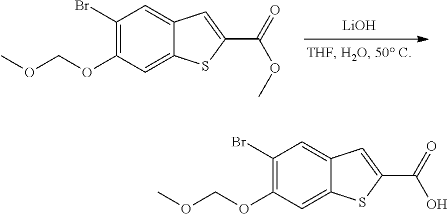 [SMILES: COCOc1cc2sc(C(=O)OC)cc2cc1Br ; "LiOH" ; COCOc1cc2sc(C(=O)O)cc2cc1Br]

LiOH (0.18 g, 7.5162 mmol) was added to a solution of methyl 5-bromo-6-(methoxymethoxy) benzothiophene-2-carboxylate (1.10 g, 3.3214 mmol) in THF (40 mL) and Water (10 mL) at 25° C. The reaction mixture was stirred for 2 h at 50° C. The reaction mixture was evaporated under reduced pressure. The residue was diluted with water and acidified pH=3 with HCl (aq., 1 M). The precipitate was filtered and filtrate cake was washed with water (20 mL). The filtrate cake was dried under vacuo at 60° C. There was 5-bromo-6-(methoxymethoxy) benzothiophene-2-carboxylic acid (1.04 g, 3.2791 mmol, 98.7268% yield) obtained as a white solid. LCMS: (ESI, m/z): [M−H]—=314.900. ¹H NMR (400 MHz, DMSO-d₆) δ 13.49 (s, 1H), 8.30 (s, 1H), 7.99 (s, 1H), 7.86 (s, 1H), 5.39 (s, 2H), 3.44 (s, 3H).

### Step d: tert-butyl 3-(5-bromo-6-(methoxymethoxy)benzo[b]thiophen-2-yl)-3-oxopropanoate

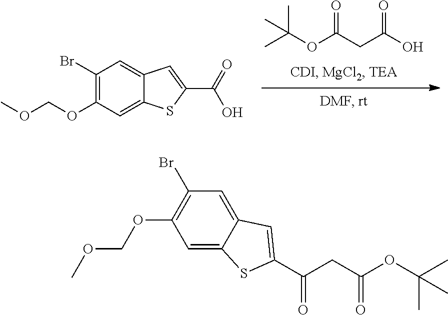

CDI (1.13 g, 6.9689 mmol) was added to a solution of 5-bromo-6-(methoxymethoxy) benzothiophene-2-carboxylic acid (1.01 g, 3.1846 mmol) in DMF (20 mL) at 25° C. The reaction mixture was stirred for 2 h at 25° C. under nitrogen atmosphere. Magnesium chloride (0.64 g, 6.7219 mmol), 3-tert-Butoxy-3-oxopropanoic acid (0.81 g, 5.0572 mmol) and TEA (1.06 g, 10.4754 mmol) were added to the reaction mixture. The reaction mixture was stirred overnight at 25° C. The reaction mixture was concentrated and diluted with EA (100 mL), washed with saturated aqueous NaHCO₃ solution (2×50 mL) and brine (50 mL). The organics dried over Na₂SO₄, filtered and evaporated under reduced pressure. The residue was purified on silica gel column EA/n-Hexane (0-20%). The pure fraction was concentrated and dried under vacuo. There was tert-butyl 3-(5-bromo-6-(methoxymethoxy)benzo[b]thiophen-2-yl)-3-oxopropanoate (1.12 g, 2.6969 mmol, 84.6855% yield) obtained as a yellow solid. LCMS: (ESI, m/z): [M−H]—=412.950. ¹H NMR (400 MHz, DMSO-d₆) δ 8.33 (s, 1H), 8.22 (s, 1H), 7.87 (s, 1H), 5.39 (d, J=10.0 Hz, 2H), 4.06 (d, J=8.4 Hz, 2H), 3.45 (s, 3H), 1.40 (s, 9H).

### Step e: 1-(tert-butyl) 4-ethyl 2-(5-bromo-6-(methoxymethoxy)benzo[b]thiophene-2-carbonyl)succinate

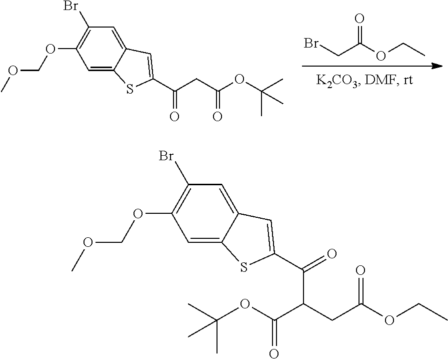

Ethyl 2-bromoacetate (0.356 g, 2.1317 mmol) was added to a solution of tert-butyl 3-(5-bromo-6-(methoxymethoxy)benzo[b]thiophen-2-yl)-3-oxopropanoate (0.891 g, 2.1454 mmol) and Potassium carbonate (0.587 g, 4.2473 mmol) in DMF (10 mL) at 25° C. The reaction mixture was stirred overnight at 25° C. under nitrogen atmosphere. The reaction mixture was concentrated and diluted with EA (200 mL), washed with water (100 mL) and brine (100 mL). The organics dried over Na₂SO₄, filtered and evaporated under reduced pressure. The residue was purified on silica gel column EA/n-Hexane (0-50%). The pure fraction was concentrated and dried under vacuo. There was 1-(tert-butyl) 4-ethyl 2-(5-bromo-6-(methoxymethoxy)benzo[b]thiophene-2-carbonyl)succinate (0.771 g, 1.5377 mmol, 71.6743% yield) obtained as a yellow oil. LCMS: (ESI, m/z): [M+H]⁺=501.050.

### Step f: ethyl 4-(5-bromo-6-hydroxy-benzothiophen-2-yl)-4-oxo-butanoate

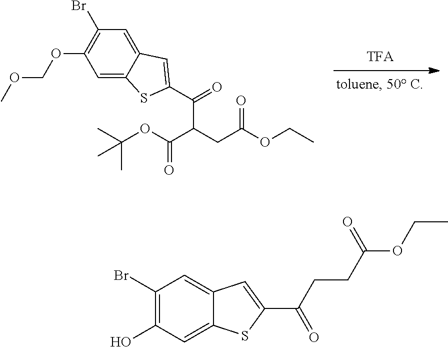 [SMILES: CCOC(=O)CC(C(=O)OC(C)(C)C)C(=O)c1cc2cc(Br)c(OCOC)cc2s1 ; CCOC(=O)CCC(=O)c1cc2cc(Br)c(O)cc2s1]

Trifluoroacetic acid (3 mL) was added to a solution of 1-(tert-butyl) 4-ethyl 2-(5-bromo-6-(methoxymethoxy)benzo[b]thiophene-2-carbonyl)succinate (0.750 g, 1.4959 mmol) in toluene (9 mL) at 25° C. The reaction mixture was heated to 50° C. and stirred for 2 h. The reaction mixture was evaporated under reduced pressure. The residue was purified on C18 column MeCN/water (0-50%). The pure fraction was concentrated and dried under vacuo. There was ethyl 4-(5-bromo-6-hydroxy-benzothiophen-2-yl)-4-oxo-butanoate (0.431 g, 1.2065 mmol, 80.6593% yield) obtained as a white solid. LCMS: (ESI, m/z): [M+H]⁺=357.000.

### Step g: ethyl 4-(5-bromo-6-(methoxymethoxy)benzo[b]thiophen-2-yl)-4-oxobutanoate

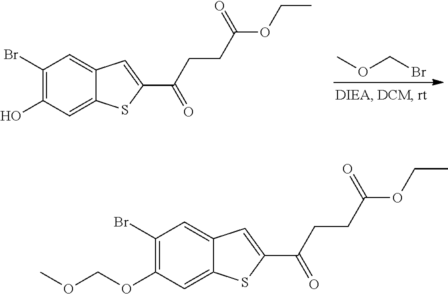

Bromomethyl methyl ether (0.198 g, 1.5845 mmol) was added to a solution of ethyl 4-(5-bromo-6-hydroxy-benzothiophen-2-yl)-4-oxo-butanoate (0.427 g, 1.1953 mmol) and DIEA (0.235 g, 1.8183 mmol) in DCM (10 mL) at 25° C. The reaction mixture was stirred for 1 h at 25° C. The reaction mixture was concentrated and diluted with DCM (100 mL), washed with water (50 mL) and brine (50 mL). The organics dried over Na₂SO₄, filtered and evaporated under reduced pressure. There was ethyl 4-(5-bromo-6-(methoxymethoxy)benzo[b]thiophen-2-yl)-4-oxobutanoate (0.503 g, 1.1282 mmol, 94.3798% yield) obtained as a yellow solid. LCMS: (ESI, m/z): [M+H]=401.100.

### Step h: ethyl 4-(6-(methoxymethoxy)-5-(4,4,5,5-tetramethyl-1,3,2-dioxaborolan-2-yl)benzo[b]thiophen-2-yl)-4-oxobutanoate

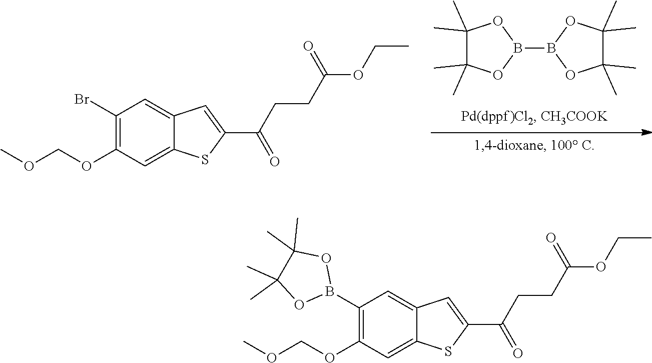

A mixture of [1,1′-Bis(diphenylphosphino)ferrocene]dichloropalladium(II) (0.110 g, 150.3341 mol), ethyl 4-(5-bromo-6-(methoxymethoxy)benzo[b]thiophen-2-yl)-4-oxobutanoate (0.478 g, 1.1912 mmol), 4,4,4′,4′,5,5,5′,5′-Octamethyl-2,2′-bi(1,3,2-dioxaborolane) (0.491 g, 1.9335 mmol) and Potassium Acetate (0.389 g, 3.9636 mmol) dissolve in 1,4-Dioxane (10 mL) at 25° C. The reaction mixture was heated to 100° C. and stirred for 4.5 h under N₂ atmosphere. The reaction mixture was concentrated and diluted with EA (200 mL), washed with water (100 mL) and brine (100 mL). The organics dried over Na₂SO₄, filtered and evaporated under reduced pressure. The residue was purified on silica gel column EA/n-Hexane (0-50%). The pure fraction was concentrated and dried under vacuo. There was ethyl 4-(6-(methoxymethoxy)-5-(4,4,5,5-tetramethyl-1,3,2-dioxaborolan-2-yl)benzo[b]thiophen-2-yl)-4-oxobutanoate (0.420 g, 936.7968 mol, 78.6423% yield) obtained as a yellow oil. LCMS: (ESI, m/z): [M+H]⁺=449.200.

### Step i: ethyl 4-(5-hydroxy-6-(methoxymethoxy)benzo[b]thiophen-2-yl)-4-oxobutanoate

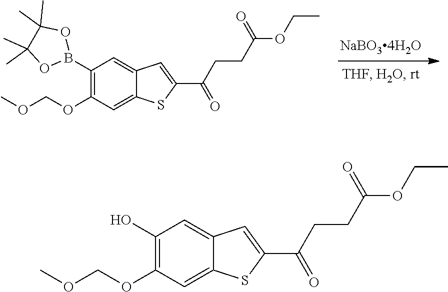

Sodium perborate tetrahydrate (0.200 g, 1.2999 mmol) was added to a solution of ethyl 4-(6-(methoxymethoxy)-5-(4,4,5,5-tetramethyl-1,3,2-dioxaborolan-2-yl)benzo[b]thiophen-2-yl)-4-oxobutanoate (0.407 g, 907.8013 mol) in THF (5 mL) and Water (5 mL) at 25° C. The reaction mixture was stirred for 2 h at 25° C. The reaction mixture was evaporated under reduced pressure. The residue was purified on C18 column MeCN/water (0-50%). The pure fraction was concentrated and dried under vacuo. There was ethyl 4-(5-hydroxy-6-(methoxymethoxy)benzo[b]thiophen-2-yl)-4-oxobutanoate (0.250 g, 738.8257 mol, 81.3863% yield) obtained as a yellow solid. LCMS: (ESI, m/z): [M+H]⁺=339.100.

### Step j: ethyl 4-(5-methoxyisoindolin-2-yl)-4-oxo-butanoate

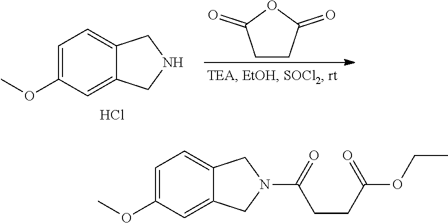 [SMILES: COc1ccc2c(c1)CNC2 ; O=C1CCC(=O)O1 ; Cl ; CCOC(=O)CCC(=O)N1Cc2ccc(OC)cc2C1]

Succinic anhydride (4.40 g, 43.9681 mmol) was added to a solution of 5-Methoxyisoindoline hydrochloride (4.89 g, 26.3399 mmol) and TEA (4.73 g, 46.7440 mmol) in Ethanol (200 mL) at 25° C. The reaction mixture was stirred for 1 h at 25° C. Thionyl chloride (20 mL) was added to the solution aboved at 0° C. The reaction mixture was stirred for 3 h at 25° C. The reaction mixture was evaporated under reduced pressure. The residue was diluted with EA (200 mL), washed with water (100 mL) and brine (50 mL). The organics dried over Na₂SO₄, filtered and evaporated under reduced pressure. There was ethyl 4-(5-methoxyisoindolin-2-yl)-4-oxo-butanoate (17.50 g, 31.5526 mmol, 119.7901% yield) obtained as a yellow solid. LCMS: (ESI, m/z): [M+H]⁺=278.150. ¹H NMR (400 MHz, DMSO-d₆) δ 7.28-7.21 (m, 1H), 6.94 (s, 0.5H), 6.92 (s, 0.5H), 6.88 (s, 0.5H), 6.86 (s, 0.5H), 4.81 (s, 1H), 4.76 (s, 1H), 4.58 (s, 1H), 4.54 (s, 1H), 4.02-3.95 (m, 2H), 3.75 (s, 3H), 2.65-2.58 (m, 2H), 2.58-2.54 (m, 2H), 1.17 (t, J=3.5 Hz, 3H).

### Step k: ethyl 4-(5-bromo-6-methoxy-isoindolin-2-yl)-4-oxo-butanoate

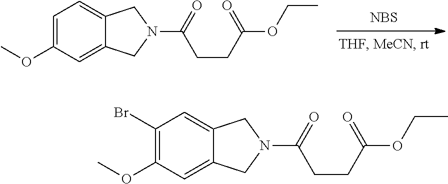

NBS (10.51 g, 59.0503 mmol) was added to a solution of ethyl 4-(5-methoxyisoindolin-2-yl)-4-oxo-butanoate (17.50 g, 31.5526 mmol) in THF (100 mL) and MeCN (100 mL) at 25° C. The reaction mixture was stirred overnight at 25° C. The reaction mixture was evaporated under reduced pressure. The residue was diluted with DCM (500 mL), washed with saturated aqueous NaHCO₃ solution (2×300 mL) and brine (200 mL). The organics dried over Na₂SO₄, filtered and evaporated under reduced pressure. The residue was purified on silica gel column MeOH/DCM (0-10%). The pure fraction was concentrated and dried under vacuo. There was ethyl 4-(5-bromo-6-methoxy-isoindolin-2-yl)-4-oxo-butanoate (7.26 g, 20.3812 mmol, 64.5943% yield) obtained as a yellow solid. LCMS: (ESI, m/z): [M+H]⁺=356.000. ¹H NMR (400 MHz, DMSO-d₆) δ 7.58 (s, 0.5H), 7.57 (s, 0.5H), 7.15 (s, 0.5H), 7.12 (s, 0.5H), 4.80 (s, 1H), 4.77 (s, 1H), 4.57 (s, 1H), 4.54 (s, 1H), 4.09-4.01 (m, 2H), 3.84 (s, 3H), 2.65-2.59 (m, 2H), 2.56-2.53 (m, 2H), 1.18 (t, J=7.1 Hz, 3H).

### Step 1: ethyl 4-(5-methoxy-6-(4,4,5,5-tetramethyl-1,3,2-dioxaborolan-2-yl)isoindolin-2-yl)-4-oxobutanoate

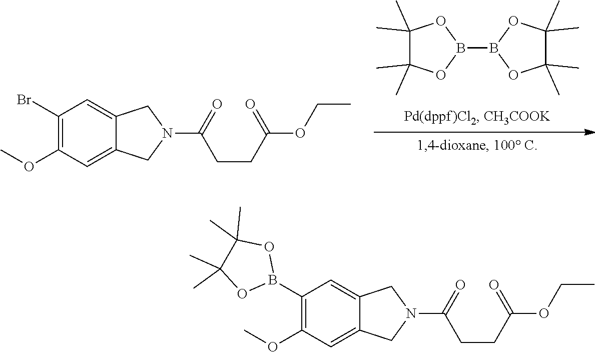

A mixture of Potassium Acetate (3.74 g, 38.1080 mmol), [1,1′-Bis(diphenylphosphino)ferrocene]dichloropalladium(II) (0.72 g, 984.0050 mol), ethyl 4-(5-bromo-6-methoxy-isoindolin-2-yl)-4-oxo-butanoate (3.64 g, 10.2187 mmol) and 4,4,4′,4′,5,5,5′,5′-Octamethyl-2,2′-bi(1,3,2-dioxaborolane) (3.84 g, 15.1218 mmol) dissolve in 1,4-Dioxane (100 mL) at 25° C. The reaction mixture was heated to 100° C. and stirred overnight under N₂ atmosphere. The reaction mixture was diluted with EA (400 mL). The precipitate was filtrated by celite, the filtrate cake was washed with EA (1×100 mL). The combined filtrate was evaporated under reduced pressure.

The residue was purified on silica gel column EA/n-Hexane (0-100%). The pure fraction was concentrated and dried under vacuo. There was ethyl 4-(5-methoxy-6-(4,4,5,5-tetramethyl-1,3,2-dioxaborolan-2-yl)isoindolin-2-yl)-4-oxobutanoate (2.70 g, 6.6952 mmol, 65.5190% yield) obtained as a yellow solid. LCMS: (ESI, m/z): [M+H]⁺=404.200. ¹H NMR (400 MHz, DMSO-d₆) δ 7.49 (s, 0.5H), 7.48 (s, 0.5H), 6.98 (s, 0.5H), 6.95 (s, 0.5H), 4.83 (s, 1H), 4.74 (s, 1H), 4.60 (s, 1H), 4.53 (s, 1H), 4.09-4.01 (m, 2H), 3.74 (t, J=6.2 Hz, 3H), 2.65-2.58 (m, 2H), 2.58-2.52 (m, 2H), 1.27 (s, 12H), 1.21-1.13 (m, 3H).

### Step m: ethyl 4-(5-hydroxy-6-methoxy-isoindolin-2-yl)-4-oxo-butanoate

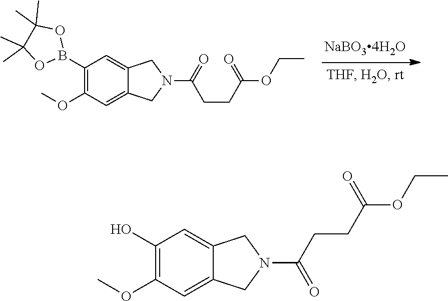

Sodium perborate tetrahydrate (1.00 g, 6.4994 mmol) was added to a solution of ethyl 4-(5-methoxy-6-(4,4,5,5-tetramethyl-1,3,2-dioxaborolan-2-yl)isoindolin-2-yl)-4-oxobutanoate (2.45 g, 6.0752 mmol) in THF (20 mL) and Water (20 mL) at 25° C. The reaction mixture was stirred for 1 h at 20° C. The reaction mixture was evaporated under reduced pressure. The reaction mixture was concentrated and diluted with DCM (500 mL), washed with saturated aqueous NaHCO₃ solution (2×300 mL) and brine (200 mL). The organics dried over Na₂SO₄, filtered and evaporated under reduced pressure. The residue was purified on silica gel column MeOH/DCM (0-10%). The pure fraction was concentrated and dried under vacuo. There was ethyl 4-(5-hydroxy-6-methoxy-isoindolin-2-yl)-4-oxo-butanoate (1.37 g, 4.6708 mmol, 76.8818% yield) obtained as a yellow solid.

LCMS: (ESI, m/z): [M+H]⁺=294.100. ¹H NMR (400 MHz, DMSO-d₆) δ 8.97 (s, 0.5H), 8.96 (s, 0.5H), 6.91 (s, 0.5H), 6.89 (s, 0.5H), 6.73 (s, 0.5H), 6.73 (s, 0.5H), 4.71 (s, 1H), 4.69 (s, 1H), 4.50 (s, 1H), 4.47 (s, 1H), 4.08-4.00 (m, 2H), 3.75 (s, 3H), 2.64-2.58 (m, 2H), 2.56-2.53 (m, 2H), 1.18 (t, J=7.1 Hz, 3H).

### Step n: ethyl 4-(5-(3-bromopropoxy)-6-methoxyisoindolin-2-yl)-4-oxobutanoate

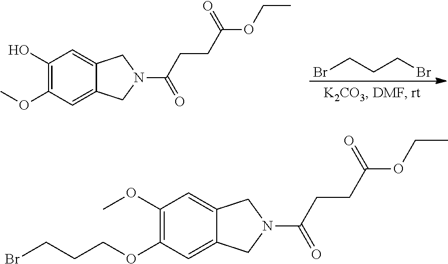 [SMILES: CCOC(=O)CCC(=O)N1Cc2cc(O)c(OC)cc2C1 ; BrCCCBr ; CCOC(=O)CCC(=O)N1Cc2cc(OC)c(OCCCBr)cc2C1]

1,3-Dibromopropane (0.73 g, 3.6159 mmol) was added to a solution of ethyl 4-(5-hydroxy-6-methoxy-isoindolin-2-yl)-4-oxo-butanoate (0.72 g, 2.4547 mmol) and Potassium carbonate (0.86 g, 6.2226 mmol) in DMF (10 mL). The reaction mixture was stirred overnight at 50° C. The reaction mixture was diluted with EA (100 mL), and washed sequentially with water (2×100 mL) and saturated brine (100 mL). The organic layer was dried over Na₂SO₄, filtered and evaporated to afford crude product. The crude product was purified by flash silica chromatography, elution gradient 0 to 100% Ethyl acetate in heptane. There was ethyl 4-(5-(3-bromopropoxy)-6-methoxyisoindolin-2-yl)-4-oxobutanoate (0.43 g, 1.0379 mmol, 42.2829% yield) obtained as a white solid. LCMS: (ESI, m/z): [M+H]⁺=414.050. ¹H NMR (400 MHz, DMSO-d₆) δ 7.01-6.93 (m, 2H), 4.75 (s, 2H), 4.53 (s, 2H), 4.10-4.00 (m, 4H), 3.76 (s, 3H), 3.71-3.62 (m, 2H), 2.65-2.58 (m, 2H), 2.58-2.53 (m, 2H), 2.28-2.18 (m, 2H), 1.18 (t, J=7.1 Hz, 3H).

### Step o: ethyl 4-(5-(3-((2-(4-ethoxy-4-oxobutanoyl)-6-(methoxymethoxy)benzo[b]thiophen-5-yl)oxy)propoxy)-6-methoxyisoindolin-2-yl)-4-oxobutanoate

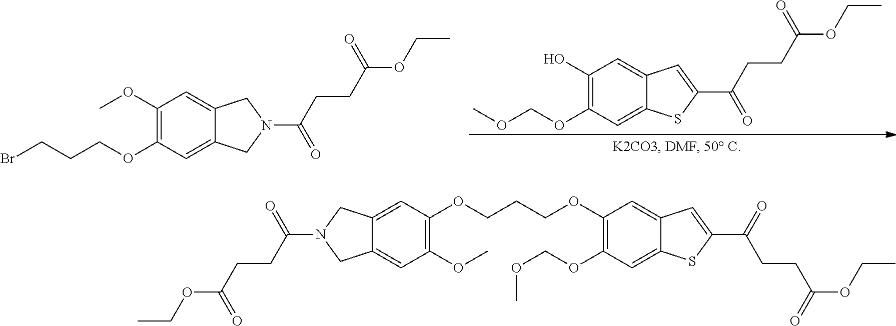 [SMILES: CCOC(=O)CCC(=O)N1Cc2cc(OC)c(OCCCBr)cc2C1 ; CCOC(=O)CCC(=O)c1cc2cc(O)c(OCOC)cc2s1 ; CCOC(=O)CCC(=O)c1cc2cc(OCCCOc3cc4c(cc3OC)CN(C(=O)CCC(=O)OCC)C4)c(OCOC)cc2s1]

Ethyl 4-[5-hydroxy-6-(methoxymethoxy) benzothiophen-2-yl]-4-oxo-butanoate (0.101 g, 298.4856 mol) was added to a solution of ethyl 4-(5-(3-bromopropoxy)-6-methoxyisoindolin-2-yl)-4-oxobutanoate (0.127 g, 306.5486 mol) and Potassium carbonate (0.142 g, 1.0275 mmol) in DMF (5 mL) at 25° C. The reaction mixture was heated to 50° C. and stirred overnight. The reaction mixture was concentrated and diluted with EA (200 mL), washed with water (100 mL) and brine (100 mL). The organics dried over Na₂SO₄, filtered and evaporated under reduced pressure. The residue was purified on silica gel column EA/n-Hexane (0-100%). The pure fraction was concentrated and dried under vacuo. There was ethyl 4-(5-(3-((2-(4-ethoxy-4-oxobutanoyl)-6-(methoxymethoxy)benzo[b]thiophen-5-yl)oxy)propoxy)-6-methoxyisoindolin-2-yl)-4-oxobutanoate (0.055 g, 81.8754 mol, 27.4303% yield) obtained as a yellow solid. LCMS: (ESI, m/z): [M+H]⁺=672.250.

### Step p: ethyl 4-(5-(3-((2-(4-ethoxy-4-oxobutanoyl)-6-hydroxybenzo[b]thiophen-5-yl)oxy)propoxy)-6-methoxyisoindolin-2-yl)-4-oxobutanoate

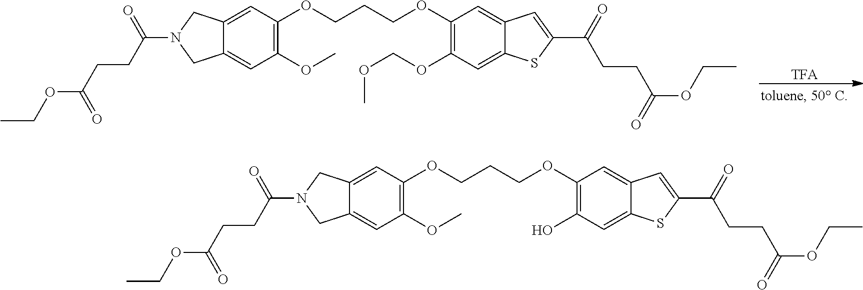

Trifluoroacetic acid (1.0 mL, 13.4622 mmol) was added to a solution of ethyl 4-(5-(3-((2-(4-ethoxy-4-oxobutanoyl)-6-(methoxymethoxy)benzo[b]thiophen-5-yl)oxy)propoxy)-6-methoxyisoindolin-2-yl)-4-oxobutanoate (0.054 g, 80.3867 mol) in toluene (2 mL) at 25° C. The reaction mixture was heated to 50° C. and stirred for 30 min. The reaction mixture was evaporated under reduced pressure. The residue was diluted with water and neutralized pH=8 with saturated aqueous NaHCO₃ solution. The resulting mixture was extracted with EA (100 mL), washed with water (50 mL) and brine (50 mL). The organics dried over Na₂SO₄, filtered and evaporated under reduced pressure. There was ethyl 4-(5-(3-((2-(4-ethoxy-4-oxobutanoyl)-6-hydroxybenzo[b]thiophen-5-yl)oxy)propoxy)-6-methoxyisoindolin-2-yl)-4-oxobutanoate (0.057 g, 77.1865 mol, 96.0190% yield) obtained as a yellow solid. LCMS: (ESI, m/z): [M+H]⁺=628.200.

### Step q: sodium 4-(5-(3-((2-(3-carboxylatopropanoyl)-6-hydroxybenzo[b]thiophen-5-yl)oxy)propoxy)-6-methoxyisoindolin-2-yl)-4-oxobutanoate

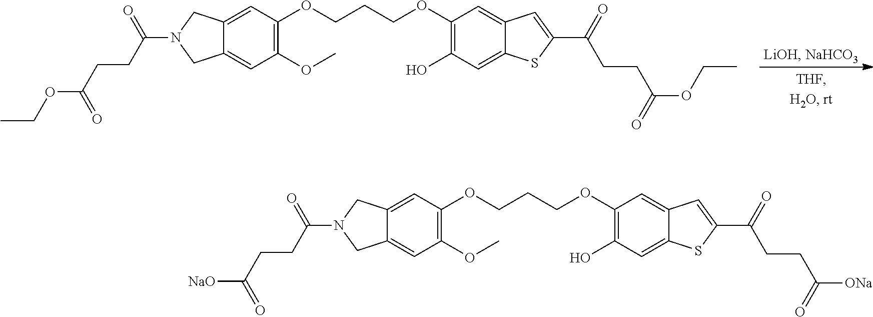

LiOH (0.023 g, 960.4022 mol) was added to a solution of ethyl 4-(5-(3-((2-(4-ethoxy-4-oxobutanoyl)-6-hydroxybenzo[b]thiophen-5-yl)oxy)propoxy)-6-methoxyisoindolin-2-yl)-4-oxobutanoate (0.055 g, 87.6215 mol) in THF (4 mL) and Water (1 mL) at 25° C. The reaction mixture was stirred overnight at 25° C. The reaction mixture was evaporated under reduced pressure. The residue was acidified pH=4 with HCl (1 M). The reaction mixture was evaporated under reduced pressure to obtain a crude product. The crude product was purified by Prep-HPLC with the following conditions: (CXTH LC6000, HPLC-P1): Column, Daisogel-C18, 50 mm*250 mm, 10 um; mobile phase, Water (0.1% TFA) and MeCN-(15-40-60% B(2-30-60 min); Detector, uv 216 nm). The solvent was removed by lyophilization. NaHCO₃ (0.004 g, 47.6153 mol) was added to the lyophilized product in water, and stirred for 30 min at 25° C. The solvent was removed by lyophilization. There was sodium 4-(5-(3-((2-(3-carboxylatopropanoyl)-6-hydroxybenzo[b]thiophen-5-yl)oxy)propoxy)-6-methoxyisoindolin-2-yl)-4-oxobutanoate (0.012 g, 19.4945 mol, 22.2485% yield) obtained as a light yellow solid. LCMS: (ESI, m/z): [M+H]⁺=572.150. ¹H NMR (400 MHz, DMSO-d₆) δ 8.00 (s, 0.5H), 7.97 (s, 0.5H), 7.39 (s, 0.5H), 7.37 (s, 0.5H), 7.30 (s, 0.5H), 7.28 (s, 0.5H), 6.99-6.87 (m, 2H), 4.74 (s, 1H), 4.70 (s, 1H), 4.49 (s, 1H), 4.47 (s, 1H), 4.20-4.08 (m, 4H), 3.73 (s, 3H), 3.07 (t, J=6.8 Hz, 2H), 2.47-2.40 (m, 2H), 2.36-2.16 (m, 6H).

### Example II-8

### sodium 4-(5-(3-((2-(3-carboxylatopropanoyl)-6-methoxyisoindolin-5-yl)oxy)propoxy)-6-methoxythieno[3,2-b]pyridin-2-yl)-4-oxobutanoate

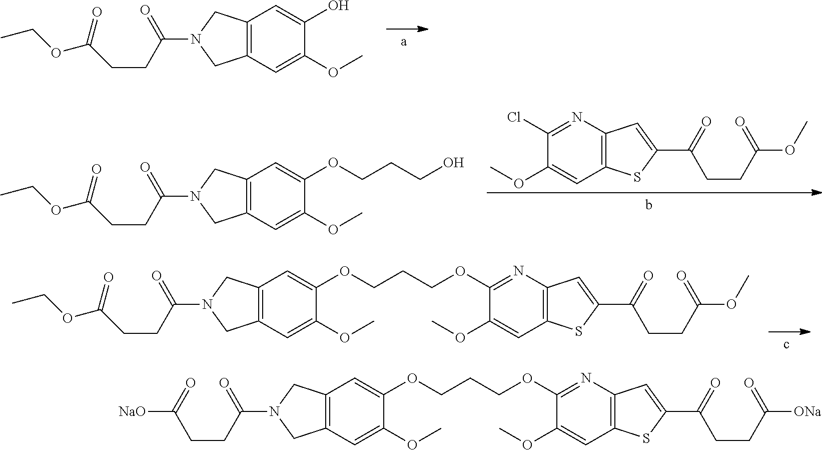

Step a: (3-Bromopropoxy)-tert-butyldimethylsilane (518 mg, 2.0456 mmol) and K₂CO₃ (320 mg, 2.3154 mmol) was added to a solution of ethyl 4-(5-hydroxy-6-methoxyisoindolin-2-yl)-4-oxobutanoate (200 mg, 681.8624 μmol) in N,N-Dimethylformamide (8 mL) at 25° C. The reaction mixture was stirred for 2 h at 25° C. Tetrabutylammonium fluoride (178 mg, 681.8624 mol) was added to the mixture. The reaction mixture was stirred for 2 h at 25° C. The reaction mixture was concentrated and diluted with EA (100 mL), washed with NaHCO₃ (aq)(100 mL) and brine (50 mL). The organics dried over Na₂SO₄, filtered and evaporated under reduced pressure. The residue was purified on silica gel column MeOH/DCM (0-10%). The pure fraction was concentrated and dried under vacuo. There was ethyl 4-(5-(3-hydroxypropoxy)-6-methoxyisoindolin-2-yl)-4-oxobutanoate (106 mg, 301.6564 mol, 44.2401% yield) obtained as a white solid. LCMS: (ESI, m/z): [M+H]⁺=352.168. ¹H NMR (400 MHz, DMSO-d₆) δ 8.31 (s, 1H), 6.95 (d, J=9.4 Hz, 2H), 4.74 (s, 2H), 4.53 (s, 2H), 4.00 (t, J=6.1 Hz, 2H), 3.75 (s, 3H), 3.20-3.13 (m, 2H), 2.63-2.51 (m, 4H), 1.57 (s, 2H), 1.38-1.21 (m, 2H), 0.94 (t, J=7.1 Hz, 3H).

Step b: K₃PO₄ (151 mg, 711.3706 mol) and RockPhos Palladacycle Gen.3 (11 mg, 13.1041 mol) was added to a solution of ethyl 4-(5-(3-hydroxypropoxy)-6-methoxyisoindolin-2-yl)-4-oxobutanoate (50 mg, 142.2907 mol) and methyl 4-(5-chloro-6-methoxy-thieno[3,2-b]pyridin-2-yl)-4-oxo-butanoate (66 mg, 210.3544 mol) in Toluene (10 mL) at 25° C. The reaction mixture was stirred at 130° C. for 16 h under N₂ atmosphere. The reaction mixture was purified on C-18 column MeCN/water (0-100%). The pure fraction was concentrated and dried under vacuo. There was ethyl 4-(5-methoxy-6-(3-((6-methoxy-2-(4-methoxy-4-oxobutanoyl)thieno[3,2-b]pyridin-5-yl)oxy)propoxy)isoindolin-2-yl)-4-oxobutanoate (4.1 mg, 6.5215 mol, 4.5832% yield) obtained as a yellow oil.

LCMS: (ESI, m/z): [M+H]⁺=629.209.

Step c: LiOH (0.006 g, 250.5397 mol) was added to a solution of ethyl 4-(5-methoxy-6-(3-((6-methoxy-2-(4-methoxy-4-oxobutanoyl)thieno[3,2-b]pyridin-5-yl)oxy)propoxy)isoindolin-2-yl)-4-oxobutanoate (0.013 g, 20.6780 mol) in Tetrahydrofuran (4 mL) and Water (1 mL) at 25° C. The reaction mixture was stirred for 2 h at 25° C. The reaction mixture was evaporated under reduced pressure. The residue was concentrated and diluted with water (2 mL). The resulting mixture was adjusted to pH=7 with HCl (1 M) at 25° C. and evaporated under reduced pressure. The residue was purified by HPLC. The pure fraction was concentrated and dried by lyophilization to obtain the free acid. NaHCO₃ (0.001 g, 11.9038 mol) was added to a mixture of the free acid in Acetonitrile (2 mL) and Water (1 mL) at 25° C. The mixture was stirred at 25° C. for 1 h and dried by lyophilization. There was sodium 4-(5-(3-((2-(3-carboxylatopropanoyl)-6-methoxyisoindolin-5-yl)oxy)propoxy)-6-methoxythieno[3,2-b]pyridin-2-yl)-4-oxobutanoate (0.0043 g, 6.8192 mol, 32.9781% yield) obtained as a white solid. LCMS: (ESI, m/z): [M+H]⁺=587.162. ¹H NMR (400 MHz, DMSO-d₆) δ 8.07 (s, 0.5H), 8.03 (s, 0.5H), 7.96 (s, 1H), 7.02-6.93 (m, 2H), 4.80-4.73 (m, 2H), 4.50 (s, 4H), 4.13 (s, 2H), 3.88 (s, 3H), 3.75-3.73 (m, 3H), 3.08 (s, 2H), 2.43 (s, 2H), 2.24 (s, 4H), 2.15 (s, 2H).

### Example II-9

### 4-(5-(3-((6-bromo-2-(3-carboxypropanoyl)benzo[b]thiophen-5-yl)oxy)propoxy)-6-methoxyisoindolin-2-yl)-4-oxobutanoic acid

### Step a: ethyl 4-(5-(3-((6-bromo-2-(4-ethoxy-4-oxobutanoyl)benzo[b]thiophen-5-yl)oxy)propoxy)-6-methoxyisoindolin-2-yl)-4-oxobutanoate

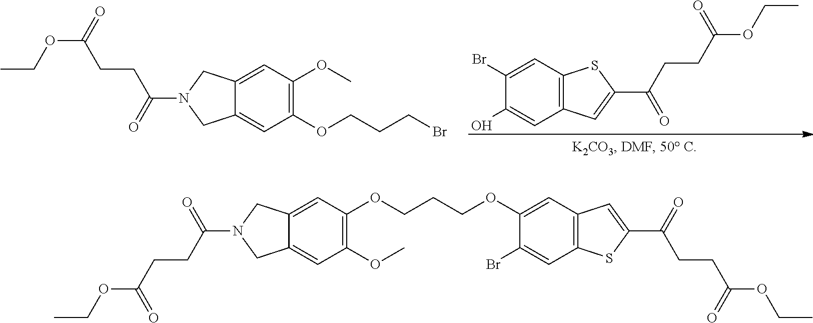

Potassium carbonate (0.080 g, 578.8482 mol) was added to a solution of ethyl 4-(5-(3-bromopropoxy)-6-methoxyisoindolin-2-yl)-4-oxobutanoate (0.075 g, 181.0327 mol) and ethyl 4-(6-bromo-5-hydroxybenzo[b]thiophen-2-yl)-4-oxobutanoate (0.071 g, 198.7576 mol) in DMF (2 mL) at 25° C. under N₂ atmosphere. The reaction mixture was heated to 50° C. and stirred overnight. The reaction mixture was purified on C18 column ACN/H₂O (0.1% FA)(0-60%). The pure fraction was concentrated and dried by lyophilization. There was ethyl 4-(5-(3-((6-bromo-2-(4-ethoxy-4-oxobutanoyl)benzo[b]thiophen-5-yl)oxy)propoxy)-6-methoxyisoindolin-2-yl)-4-oxobutanoate (0.080 g, 115.8418 mol, 63.9894% yield) obtained as a white solid.

LCMS: (ESI, m/z): [M+H]⁺=690.13. ¹H NMR (400 MHz, DMSO-d₆) δ 8.39 (s, 1H), 8.26 (s, 1H), 7.69 (s, 1H), 7.05-6.92 (m, 2H), 4.73 (s, 1H), 4.72 (s, 1H), 4.51 (d, J=3.6 Hz, 2H), 4.29 (s, 2H), 4.19 (t, J=6.4 Hz, 2H), 4.11-4.00 (m, 4H), 3.73 (d, J=2.0 Hz, 3H), 3.40-3.34 (m, 2H), 2.68 (t, J=6.3 Hz, 2H), 2.63-2.53 (m, 4H), 2.26 (t, J=6.3 Hz, 2H), 1.18 (t, J=7.1 Hz, 6H).

### Step b: 4-(5-(3-((6-bromo-2-(3-carboxypropanoyl)benzo[b]thiophen-5-yl)oxy)propoxy)-6-methoxyisoindolin-2-yl)-4-oxobutanoic acid

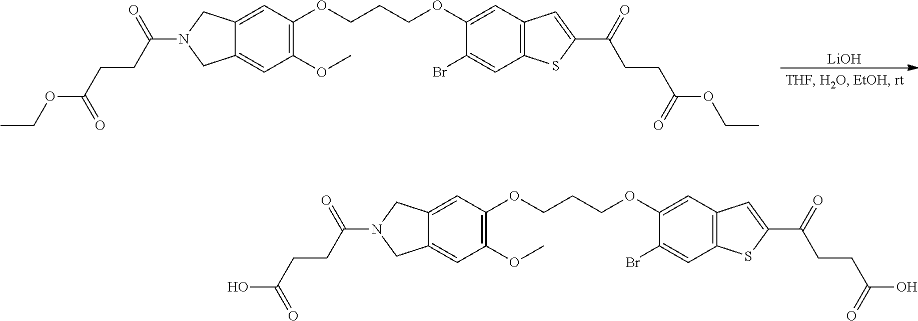 [SMILES: CCOC(=O)CCC(=O)c1cc2cc(OCCCOc3cc4c(cc3OC)CN(C(=O)CCC(=O)OCC)C4)c(Br)cc2s1 ; "LiOH" ; COc1cc2c(cc1OCCCOc1cc3cc(C(=O)CCC(=O)O)sc3cc1Br)CN(C(=O)CCC(=O)O)C2]

LiOH (0.050 g, 2.0878 mmol) was added to a solution of ethyl 4-(5-(3-((6-bromo-2-(4-ethoxy-4-oxobutanoyl)benzo[b]thiophen-5-yl)oxy)propoxy)-6-methoxyisoindolin-2-yl)-4-oxobutanoate (0.044 g, 63.7130 μmol) in THF (2 mL), EtOH (1 mL) and Water (2 mL) at 25° C. The reaction mixture was stirred for 2 h at 25° C.

The reaction mixture was adjusted to pH=3 with HCl (1 mol/L). The reaction mixture was purified on C18 column ACN/H₂O (0.1% FA)(0-55%). The pure fraction was concentrated and dried by lyophilization. There was 4-(5-(3-((6-bromo-2-(3-carboxypropanoyl)benzo[b]thiophen-5-yl)oxy)propoxy)-6-methoxyisoindolin-2-yl)-4-oxobutanoic acid (0.012 g, 18.9128 mol, 29.6844% yield) obtained as a white solid. LCMS: (ESI, m/z): [M+H]⁺=634.07. ¹H NMR (400 MHz, DMSO-d₆) δ 8.37 (s, 1H), 8.24 (d, J=3.1 Hz, 1H), 7.68 (d, J=3.8 Hz, 1H), 7.04-6.92 (m, 2H), 4.73 (s, 1H), 4.71 (s, 1H), 4.51 (d, J=4.7 Hz, 2H), 4.29 (s, 2H), 4.23-4.15 (m, 2H), 3.73 (s, 3H), 3.33-3.25 (m, 2H), 2.63-2.54 (m, 4H), 2.53-2.50 (m, 2H), 2.31-2.20 (m, 2H).

Example II-10 4-(5-(3-((2-(3-carboxypropanoyl)-4-fluoro-6-methoxyisoindolin-5-yl)oxy)propoxy)-6-methoxybenzo[b]thiophen-2-yl)-4-oxobutanoic acid

### Step a: ethyl 4-(5-(3-((2-(4-ethoxy-4-oxobutanoyl)-4-fluoro-6-methoxyisoindolin-5-yl)oxy)propoxy)-6-methoxybenzo[b]thiophen-2-yl)-4-oxobutanoate

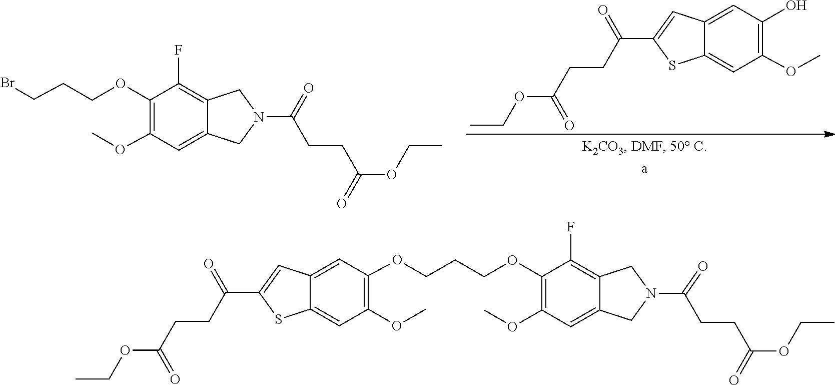

Potassium carbonate (0.099 g, 716.3246 mol) was added to a solution of ethyl 4-(5-hydroxy-6-methoxybenzo[b]thiophen-2-yl)-4-oxobutanoate (0.062 g, 201.0709 mol) and ethyl 4-(5-(3-bromopropoxy)-4-fluoro-6-methoxyisoindolin-2-yl)-4-oxobutanoate (0.069 g, 159.6186 mol) in DMF (3 mL) at 25° C. under N₂ atmosphere. The reaction mixture was stirred overnight at 50° C. The mixture was purified on C18 column ACN/H₂O (0.1% FA)(0-50%). The pure fraction was concentrated and dried further in lyophilization. There was ethyl 4-(5-(3-((2-(4-ethoxy-4-oxobutanoyl)-4-fluoro-6-methoxyisoindolin-5-yl)oxy)propoxy)-6-methoxybenzo[b]thiophen-2-yl)-4-oxobutanoate (0.071 g, 107.6218 mol, 67.4244% yield) obtained as a white solid. LCMS: (ESI, m/z): [M+H]⁺=660.220. ¹H NMR (400 MHz, DMSO-d₆) δ 8.22 (s, 1H), 7.59 (s, 1H), 7.50 (d, J=3.4 Hz, 1H), 6.92 (s, 0.5H), 6.88 (s, 0.5H), 4.80 (s, 1H), 4.78 (s, 1H), 4.57 (s, 2H), 4.27-4.20 (m, 2H), 4.17 (t, J=6.0 Hz, 2H), 4.10-4.02 (m, 4H), 3.85 (s, 3H), 3.76 (s, 3H), 3.31-3.28 (m, 2H), 2.69-2.53 (m, 6H), 2.20-2.09 (m, 2H), 1.18 (t, J=7.1 Hz, 6H).

### Step b: 4-(5-(3-((2-(3-carboxypropanoyl)-4-fluoro-6-methoxyisoindolin-5-yl)oxy)propoxy)-6-methoxybenzo[b]thiophen-2-yl)-4-oxobutanoic acid

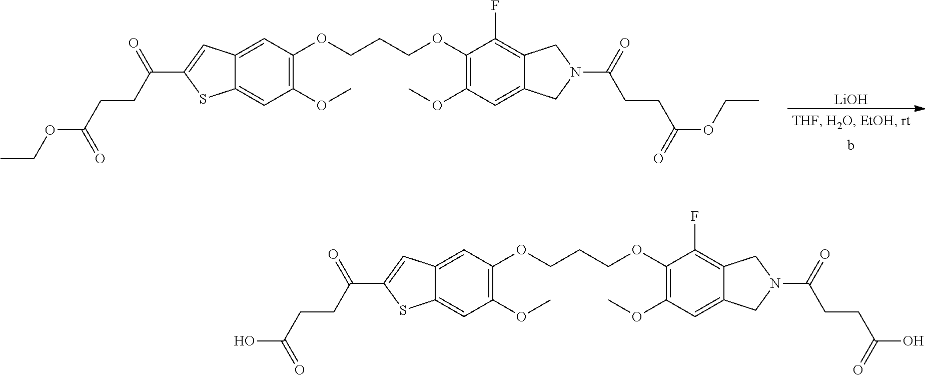 [SMILES: CCOC(=O)CCC(=O)c1cc2cc(OCCCOc3c(OC)cc4c(c3F)CN(C(=O)CCC(=O)OCC)C4)c(OC)cc2s1 ; "LiOH" ; COc1cc2sc(C(=O)CCC(=O)O)cc2cc1OCCCOc1c(OC)cc2c(c1F)CN(C(=O)CCC(=O)O)C2]

LiOH (0.044 g, 1.8373 mmol) was added to a mixture of ethyl 4-(5-(3-((2-(4-ethoxy-4-oxobutanoyl)-4-fluoro-6-methoxyisoindolin-5-yl)oxy)propoxy)-6-methoxybenzo[b]thiophen-2-yl)-4-oxobutanoate (0.100 g, 155.1150 mol) in THF (2 mL), EtOH (2 mL) and Water (2 mL) at 25° C. The reaction mixture was stirred for 2 h at 25° C. The reaction mixture was adjusted to pH=3 with HCl (1 mol/L), The mixture was evaporated under reduced pressure. The residue was purified on C18 column ACN/H₂O (0.1% FA)(0-40%). The pure fraction was concentrated and dried under vacuo. There was 4-(5-(3-((2-(3-carboxypropanoyl)-4-fluoro-6-methoxyisoindolin-5-yl)oxy)propoxy)-6-methoxybenzo[b]thiophen-2-yl)-4-oxobutanoic acid (0.026 g, 43.0741 mol, 66.0854% yield) obtained as a white solid. LCMS: (ESI, m/z): [M+H]⁺=604.160. ¹H NMR (400 MHz, DMSO-d₆) δ 8.21 (s, 1H), 7.59 (s, 1H), 7.50 (d, J=3.0 Hz, 1H), 6.91 (s, 0.5H), 6.88 (s, 0.5H), 4.79 (s, 1H), 4.77 (s, 1H), 4.56 (s, 2H), 4.27-4.20 (m, 2H), 4.17 (t, J=6.0 Hz, 2H), 3.85 (s, 3H), 3.76 (s, 3H), 3.26-3.24 (m, 2H), 2.63-2.54 (m, 4H), 2.48-2.44 (m, 2H), 2.19-2.10 (m, 2H).

### Example II-11

### 4-(5-(3-((6-(3-carboxypropanoyl)-3-methoxy-6,7-dihydro-5H-pyrrolo[3,4-b]pyridin-2-yl)oxy)propoxy)-6-methoxythieno[3,2-b]pyridin-2-yl)-4-oxobutanoic acid

### Step a: 3-methoxy-2-(3-tetrahydropyran-2-yloxypropoxy)-6,7-dihydro-5H-pyrrolo[3,4-b]pyridine

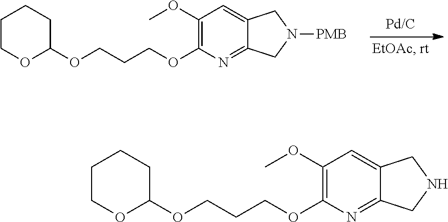

Pd/C (2.553 g, 2.3990 mmol) was added to a solution of 3-methoxy-6-(4-methoxybenzyl)-2-(3-((tetrahydro-2H-pyran-2-yl)oxy)propoxy)-6,7-dihydro-5H-pyrrolo[3,4-b]pyridine (2.464 g, 5.7500 mmol) in Ethyl acetate (60 mL) at 25° C. The reaction mixture was stirred overnight at 25° C. under H₂ atmosphere. The resulting mixture was filtered. The filter cake was washed with EA (3×100 mL). The filtrate was concentrated under reduced pressure.

There was 3-methoxy-2-(3-tetrahydropyran-2-yloxypropoxy)-6,7-dihydro-5H-pyrrolo[3,4-b]pyridine (1.936 g, 4.3947 mmol, 76.4291% yield ) obtained as a yellow oli. LCMS: (ESI, m/z): [M+H]⁺=309.174.

### Step b: 3-((3-methoxy-6,7-dihydro-5H-pyrrolo[3,4-b]pyridin-2-yl)oxy)propan-1-ol

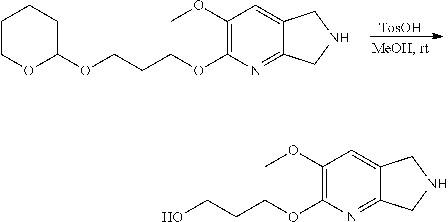 [SMILES: COc1cc2c(nc1OCCCOC1CCCCO1)CNC2 ; "TosOH" ; COc1cc2c(nc1OCCCO)CNC2]

4-methylbenzenesulfonic acid (0.69 g, 2.5430 mmol) was added to a solution of 3-methoxy-2-(3-tetrahydropyran-2-yloxypropoxy)-6,7-dihydro-5H-pyrrolo[3,4-b]pyridine (1.91 g, 4.3357 mmol) in MeOH (40 mL) at 25° C. The reaction mixture was stirred for 2 h at 25° C. The reaction mixture was evaporated under reduced pressure. The residue was purified on C18 column MeCN/water (0-50%). The pure fraction was concentrated and dried under vacuo. There was 3-((3-methoxy-6,7-dihydro-5H-pyrrolo[3,4-b]pyridin-2-yl)oxy)propan-1-ol (0.814 g, 3.6298 mmol, 83.7191% yield) obtained as a yellow oil. LCMS: (ESI, m/z): [M+H]⁺=225.116.

### Step c: ethyl 4-(2-(3-hydroxypropoxy)-3-methoxy-5,7-dihydro-6H-pyrrolo[3,4-b]pyridin-6-yl)-4-oxobutanoate

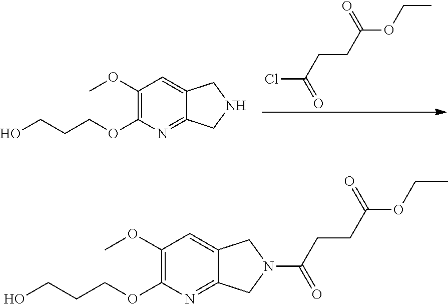

Ethyl Succinyl Chloride (0.232 g, 1.4096 mmol) was added to a solution of 3-((3-methoxy-6,7-dihydro-5H-pyrrolo[3,4-b]pyridin-2-yl)oxy)propan-1-ol (0.366 g, 1.6321 mmol) and Triethylamine (0.363 g, 3.5873 mmol) in Tetrahydrofuran (20 mL) at 0° C. under N₂ atmosphere. The reaction mixture was stirred at 25° C. for 1 h. The reaction mixture was purified on C-18 column MeCN/water (0-100%). The pure fraction was concentrated and dried under vacuo. There was ethyl 4-(2-(3-hydroxypropoxy)-3-methoxy-5,7-dihydro-6H-pyrrolo[3,4-b]pyridin-6-yl)-4-oxobutanoate (224 mg, 635.6750 mol, 38.9491% yield) obtained as a white solid. LCMS: (ESI, m/z): [M+H]⁺=353.163. ¹H NMR (400 MHz, DMSO-d₆) δ 7.38-7.26 (m, 1H), 4.81-4.73 (m, 1H), 4.68 (s, 1H), 4.54 (s, 2H), 4.42 (s, 1H), 4.30 (t, J=6.3 Hz, 2H), 4.11-3.99 (m, 2H), 3.78 (s, 3H), 3.57-3.50 (m, 2H), 2.66-2.59 (m, 2H), 2.58-2.52 (m, 2H), 1.91-1.80 (m, 2H), 1.18 (t, J=7.1 Hz, 3H).

### Step d: ethyl 4-(3-methoxy-2-(3-((6-methoxy-2-(4-methoxy-4-oxobutanoyl)thieno[3,2-b]pyridin-5-yl)oxy)propoxy)-5,7-dihydro-6H-pyrrolo[3,4-b]pyridin-6-yl)-4-oxobutanoate

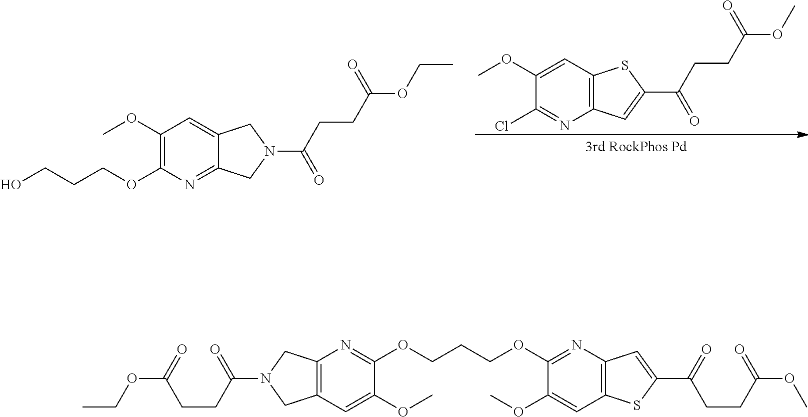

Potassium phosphate tribasic (0.212 g, 998.7454 mol) and RockPhos Palladacycle Gen.3 (0.022 g, 26.2082 μmol) was added to a solution of ethyl 4-(2-(3-hydroxypropoxy)-3-methoxy-5,7-dihydro-6H-pyrrolo[3,4-b]pyridin-6-yl)-4-oxobutanoate (0.087 g, 246.8916 mol) and methyl 4-(5-chloro-6-methoxy-thieno[3,2-b]pyridin-2-yl)-4-oxo-butanoate (0.120 g, 382.4626 mol) in Toluene (20 mL) at 25° C. The reaction mixture was stirred overnight at 25° C. under N₂ atmosphere. The reaction mixture was evaporated under reduced pressure. The residue was purified on C-18 column MeCN/water (0-100%). The pure fraction was concentrated and dried under vacuo. The residue was purified by HPLC. The pure fraction was concentrated and lyophilization. There was ethyl 4-(3-methoxy-2-(3-((6-methoxy-2-(4-methoxy-4-oxobutanoyl)thieno[3,2-b]pyridin-5-yl)oxy)propoxy)-5,7-dihydro-6H-pyrrolo[3,4-b]pyridin-6-yl)-4-oxobutanoate (20 mg, 31.7623 mol, 12.8649% yield) obtained as a white solid. LCMS: (ESI, m/z): [M+H]⁺=630.204. ¹H NMR (400 MHz, DMSO-d₆) δ 8.22 (d, J=3.6 Hz, 1H), 7.97 (d, J=4.8 Hz, 1H), 7.37-7.28 (m, 1H), 4.73 (s, 1H), 4.61 (s, 1H), 4.55-4.48 (m, 3H), 4.47-4.36 (m, 3H), 4.09-4.01 (m, 2H), 3.89 (s, 3H), 3.78 (s, 3H), 3.61 (d, J=5.6 Hz, 3H), 2.68 (d, J=6.5 Hz, 2H), 2.62-2.54 (m, 3H), 2.25 (d, J=4.2 Hz, 2H), 1.18 (t, J=7.1 Hz, 3H).

### Step e: 4-(5-(3-((6-(3-carboxypropanoyl)-3-methoxy-6,7-dihydro-5H-pyrrolo[3,4-b]pyridin-2-yl)oxy)propoxy)-6-methoxythieno[3,2-b]pyridin-2-yl)-4-oxobutanoic acid

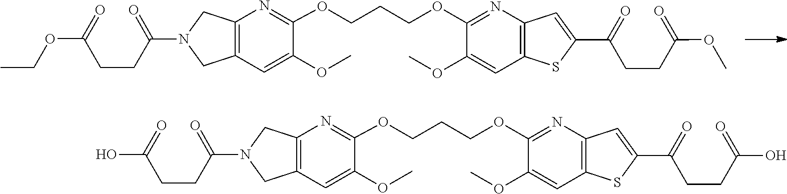 [SMILES: CCOC(=O)CCC(=O)N1Cc2cc(OC)c(OCCCOc3nc4cc(C(=O)CCC(=O)OC)sc4cc3OC)nc2C1 ; COc1cc2c(nc1OCCCOc1nc3cc(C(=O)CCC(=O)O)sc3cc1OC)CN(C(=O)CCC(=O)O)C2]

LiOH (0.004 g, 167.0265 mol) was added to a solution of ethyl 4-(3-methoxy-2-(3-((6-methoxy-2-(4-methoxy-4-oxobutanoyl)thieno [3,2-b]pyridin-5-yl)oxy)propoxy)-5,7-dihydro-6H-pyrrolo [3,4-b]pyridin-6-yl)-4-oxobutanoate (0.010 g, 15.8812 mol) in Tetrahydrofuran (4 mL) and Water (1 mL) at 25° C. The reaction mixture was stirred at 25° C. for 2 h. The reaction mixture was evaporated under reduced pressure. The residue was concentrated and diluted with water (2 mL). The resulting mixture was adjusted to pH=7 with HCl (1 M) at 25° C. and evaporated under reduced pressure. The residue was purified by HPLC. The pure fraction was concentrated and lyophilization. There was 4-(5-(3-((6-(3-carboxypropanoyl)-3-methoxy-6,7-dihydro-5H-pyrrolo[3,4-b]pyridin-2-yl)oxy)propoxy)-6-methoxythieno[3,2-b]pyridin-2-yl)-4-oxobutanoic acid (2.6 mg, 4.4248 mol, 27.8619% yield) obtained as a white solid. LCMS: (ESI, m/z): [M+H]⁺=588.157. ¹H NMR (400 MHz, DMSO-d₆) 6 8.20 (s, 1H), 7.97 (d, J=3.4 Hz, 1H), 7.35-7.28 (m, 1H), 4.73 (s, 2H), 4.61 (s, 2H), 4.51 (s, 4H), 4.47-4.33 (m, 4H), 3.88 (s, 3H), 3.78 (s, 3H), 2.71-2.61 (m, 4H), 2.26 (s, 2H).

### Example II-12 4-(5-(3-((2-(4-ethoxy-4-oxobutanoyl)-6-methoxybenzo[b]thiophen-5-yl)oxy)propoxy)-6-methoxyisoindolin-2-yl)-4-oxobutanoic acid

### Step a: tert-butyl 5-methoxyisoindoline-2-carboxylate

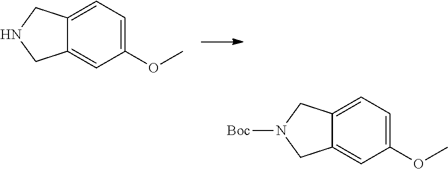 [SMILES: COc1ccc2c(c1)CNC2 ; COc1ccc2c(c1)CN(C(=O)OC(C)(C)C)C2]

TEA (33.12 g, 327.3070 mmol) was added to a solution of 5-Methoxyisoindoline hydrochloride (20.01 g, 107.7833 mmol) and Di-tert-butyl dicarbonate (35.28 g, 161.6523 mmol) in DCM (500 mL) at 20° C. The reaction mixture was stirred overnight at 20° C. The reaction mixture was evaporated under reduced pressure. The residue was purified on silica gel column EA/n-Hexane (0-50%). The pure fraction was concentrated and dried under vacuo. There was tert-butyl 5-methoxyisoindoline-2-carboxylate (30.66 g, 104.5347 mmol, 96.9859% yield) obtained as a white solid. LCMS: (ESI, m/z): [M+H-tBu+ACN]⁺=235.136. ¹H NMR (400 MHz, DMSO-d₆) δ 7.24-7.18 (m, 1H), 6.90 (d, J=4.5 Hz, 1H), 6.85 (s, 0.5H), 6.83 (s, 0.5H), 4.58-4.44 (m, 4H), 3.74 (s, 3H), 1.45 (s, 9H).

### Step b: tert-butyl 5-bromo-6-methoxy-isoindoline-2-carboxylate

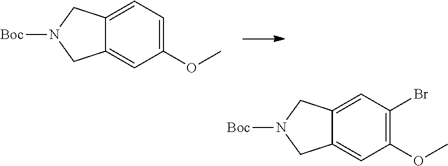 [SMILES: COc1ccc2c(c1)CN(C(=O)OC(C)(C)C)C2 ; COc1cc2c(cc1Br)CN(C(=O)OC(C)(C)C)C2]

NBS (43.64 g, 245.1906 mmol) was added to a solution of tert-butyl 5-methoxyisoindoline-2-carboxylate (30.40 g, 121.9390 mmol) in Tetrahydrofuran (300 mL) and Acetonitrile (300 mL) at 20° C. The reaction mixture was stirred overnight at 20° C. The reaction mixture was evaporated under reduced pressure. The reaction mixture was concentrated and diluted with EA (1000 mL), washed with NaHCO₃ (aq)(3×200 mL) and brine (300 mL). The organics dried over Na₂SO₄, filtered and evaporated under reduced pressure. The residue was purified on silica gel column EA/n-Hexane (0-50%). The pure fraction was concentrated and dried under vacuo. There was tert-butyl 5-bromo-6-methoxy-isoindoline-2-carboxylate (17.61 g, 53.6562 mmol, 44.0025% yield) obtained as a yellow solid. LCMS: (ESI, m/z): [M+H-tBu+ACN]⁺=313.047. ¹H NMR (400 MHz, DMSO-d₆) δ 7.54 (d, J=5.3 Hz, 1H), 7.11 (d, J=4.2 Hz, 1H), 4.51 (t, J=9.8 Hz, 4H), 3.82 (d, J=3.7 Hz, 3H), 1.45 (s, 9H).

### Step c: tert-butyl 5-methoxy-6-(4,4,5,5-tetramethyl-1,3,2-dioxaborolan-2-yl)isoindoline-2-carboxylate

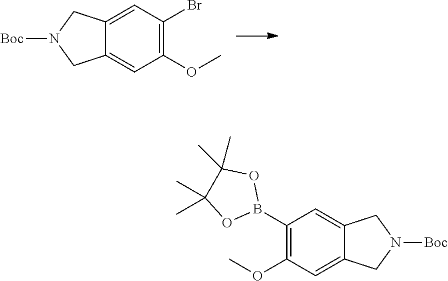 [SMILES: COc1cc2c(cc1Br)CN(C(=O)OC(C)(C)C)C2 ; COc1cc2c(cc1B1OC(C)(C)C(C)(C)O1)CN(C(=O)OC(C)(C)C)C2]

[1,1′-Bis(diphenylphosphino)ferrocene]dichloropalladium(II) (2.10 g, 2.8700 mmol), 4,4,4′,4′,5,5,5′,5′-Octamethyl-2,2′-bi(1,3,2-dioxaborolane) (11.71 g, 46.1137 mmol) and Potassium Acetate (9.76 g, 99.4475 mmol) was added to a solution of tert-butyl 5-bromo-6-methoxy-isoindoline-2-carboxylate (10.04 g, 30.5910 mmol) in 1,4-Dioxane (200 mL) at 20° C. The reaction mixture was heated to 100° C. and stirred overnight under N₂ atmosphere. The reaction mixture was concentrated and diluted with EA (500 mL), washed with water (200 mL) and brine (200 mL). The organics dried over Na₂SO₄, filtered and evaporated under reduced pressure. The residue was purified on silica gel column EA/n-Hexane (0-50%). The pure fraction was concentrated and dried under vacuo. There was tert-butyl 5-methoxy-6-(4,4,5,5-tetramethyl-1,3,2-dioxaborolan-2-yl)isoindoline-2-carboxylate (11.00 g, 29.3126 mmol, 95.8208% yield) obtained as a oil. LCMS: (ESI, m/z): [M+H-tBu+ACN]⁺=361.222.

### Step d: tert-butyl 5-hydroxy-6-methoxy-isoindoline-2-carboxylate

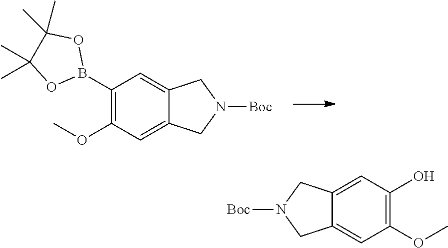 [SMILES: COc1cc2c(cc1B1OC(C)(C)C(C)(C)O1)CN(C(=O)OC(C)(C)C)C2 ; COc1cc2c(cc1O)CN(C(=O)OC(C)(C)C)C2]

Sodium perborate tetrahydrate (6.28 g, 40.8164 mmol) was added to a solution of tert-butyl 5-methoxy-6-(4,4,5,5-tetramethyl-1,3,2-dioxaborolan-2-yl)isoindoline-2-carboxylate (9.75 g, 25.9816 mmol) in Tetrahydrofura (150 mL) and Water (150 mL) at 20° C. The reaction mixture was stirred for 2 h at 20° C. The reaction mixture was concentrated and diluted with EA (600 mL), washed with water (300 mL) and brine (300 mL). The organics dried over Na₂SO₄, filtered and evaporated under reduced pressure. The residue was purified on silica gel column EA/DCM (0-100%). The pure fraction was concentrated and dried under vacuo. There was tert-butyl 5-hydroxy-6-methoxy-isoindoline-2-carboxylate (6.58 g, 24.8017 mmol, 95.4589% yield) obtained as a yellow oil. LCMS: (ESI, m/z): [M+H-tBu+ACN]⁺=251.131. ¹H NMR (400 MHz, DMSO-d₆) δ 8.92 (s, 1H), 6.88 (d, J=5.8 Hz, 1H), 6.70 (d, J=2.3 Hz, 1H), 4.44 (t, J=9.6 Hz, 4H), 3.74 (d, J=3.4 Hz, 3H), 1.44 (s, 9H).

### Step e: tert-butyl 5-(3-bromopropoxy)-6-methoxy-isoindoline-2-carboxylate

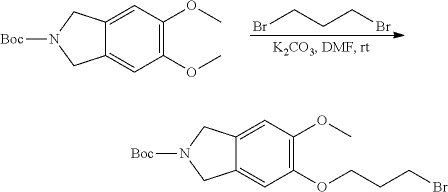

1,3-Dibromopropane (0.564 g, 2.7936 mmol) was added to a mixture of Potassium carbonate (0.286 g, 2.0694 mmol) and tert-butyl 5-hydroxy-6-methoxyisoindoline-2-carboxylate (0.179 g, 674.6977 mol) in DMF (3 mL) at 20° C. under N₂ atmosphere. The reaction mixture was stirred for 5 h at 20° C. The mixture was purified on C18 column ACN/H₂O (0.1% FA)(0-50%). The pure fraction was concentrated and dried under vacuo. There was tert-butyl 5-(3-bromopropoxy)-6-methoxy-isoindoline-2-carboxylate (0.154 g, 398.6747 mol, 59.0894% yield) obtained as a white oil. LCMS: (ESI, m/z): [M+H]⁺=386.090. ¹H NMR (400 MHz, DMSO-d₆) δ 6.96 (t, J=5.8 Hz, 2H), 4.50 (s, 2H), 4.48 (s, 2H), 4.03 (q, J=5.5 Hz, 2H), 3.74 (d, J=3.4 Hz, 3H), 3.66 (t, J=6.5 Hz, 2H), 2.27-2.19 (m, 2H), 1.45 (s, 9H).

### Step f: tert-butyl 5-(3-((2-(4-ethoxy-4-oxobutanoyl)-6-methoxybenzo[b]thiophen-5-yl)oxy)propoxy)-6-methoxyisoindoline-2-carboxylate

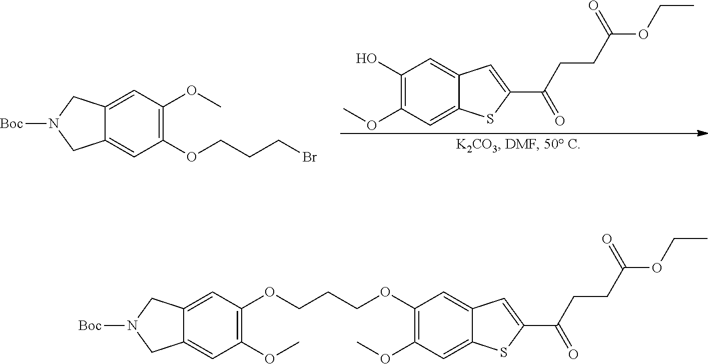 [SMILES: COc1cc2c(cc1OCCCBr)CN(C(=O)OC(C)(C)C)C2 ; CCOC(=O)CCC(=O)c1cc2cc(O)c(OC)cc2s1 ; CCOC(=O)CCC(=O)c1cc2cc(OCCCOc3cc4c(cc3OC)CN(C(=O)OC(C)(C)C)C4)c(OC)cc2s1]

Potassium carbonate (0.079 g, 571.6126 mol) was added to a solution of tert-butyl 5-(3-bromopropoxy)-6-methoxyisoindoline-2-carboxylate (0.079 g, 204.5150 mol) and ethyl 4-(5-hydroxy-6-methoxybenzo[b]thiophen-2-yl)-4-oxobutanoate (0.064 g, 207.5571 mol) in DMF (3 mL) at 20° C. under N₂ atmosphere. The reaction mixture was stirred overnight at 50° C. The reaction mixture was quenched with adding of water (50 mL) at 20° C., extracted with EA (3×50 mL) and washed with brine (50 mL). The organics dried over Na₂SO₄, filtered and evaporated under reduced pressure. There was a crude tert-butyl 5-(3-((2-(4-ethoxy-4-oxobutanoyl)-6-methoxybenzo[b]thiophen-5-yl)oxy)propoxy)-6-methoxyisoindoline-2-carboxylate (0.088 g, 143.3886 mol, 70.1115% yield) obtained as a yellow oil which was used directly in the next step. LCMS: (ESI, m/z): [M+H]=614.230.

### Step g: 4-(5-(3-((2-(4-ethoxy-4-oxobutanoyl)-6-methoxybenzo[b]thiophen-5-yl)oxy)propoxy)-6-methoxyisoindolin-2-yl)-4-oxobutanoic acid

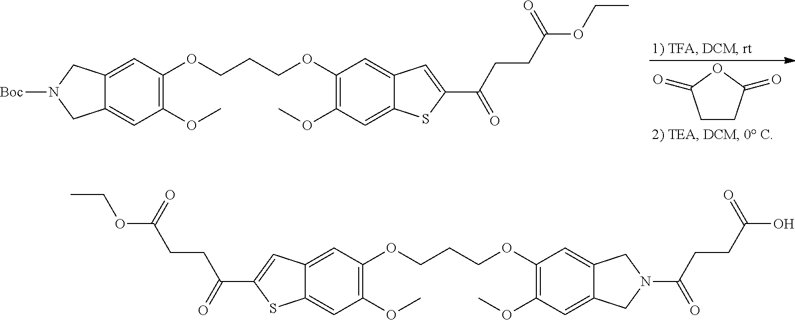

Trifluoroacetic acid (1 mL) was added to a solution of tert-butyl 5-(3-((2-(4-ethoxy-4-oxobutanoyl)-6-methoxybenzo[b]thiophen-5-yl)oxy)propoxy)-6-methoxyisoindoline-2-carboxylate (0.084 g, 136.8709 mol) in DCM (3 mL) at 20° C. The reaction mixture was stirred for 1 h at 20° C. The reaction mixture was evaporated under reduced pressure. Dihydrofuran-2,5-dione (0.050 g, 499.6373 mol) and TEA (0.195 g, 1.9271 mmol) was added to a solution of the residue in DCM (5 mL) at 0° C. under N₂ atmosphere. The reaction mixture was stirred and warmed up to 20° C. naturally. The reaction mixture was quenched with adding of water (50 mL) at 20° C. and adjusted to pH=3 with HCl (1 mol/L), extracted with EA (3×50 mL) and washed with brine (50 mL). The organics dried over Na₂SO₄, filtered and evaporated under reduced pressure. The residue was purified on C18 column ACN/H₂O (0.1% FA)(0-40%). The pure fraction was concentrated and dried under vacuo. There was 4-(5-(3-((2-(4-ethoxy-4-oxobutanoyl)-6-methoxybenzo [b]thiophen-5-yl)oxy)propoxy)-6-methoxyisoindolin-2-yl)-4-oxobutanoic acid (0.038 g, 61.9221 mol, 45.2413% yield) obtained as a white solid. LCMS: (ESI, m/z): [M+H]⁺=614.200. ¹H NMR (400 MHz, DMSO-d₆) δ 8.18 (s, 1H), 7.60 (s, 1H), 7.53 (s, 1H), 7.02 (s, 0.5H), 6.98 (s, 0.5H), 6.97 (s, 0.5H), 6.95 (s, 0.5H), 4.74 (s, 1H), 4.72 (s, 1H), 4.52 (s, 2H), 4.20 (t, J=6.3 Hz, 2H), 4.14 (t, J=6.3 Hz, 2H), 4.06 (q, J=7.1 Hz, 2H), 3.86 (s, 3H), 3.75 (s, 3H), 3.30-3.28 (m, 2H), 2.70-2.64 (m, 2H), 2.58-2.52 (m, 4H), 2.27-2.18 (m, 2H), 1.17 (t, J=7.1 Hz, 3H).

### Example II-13

### Sodium 4-(5-(3-((2-(3-carboxylatopropanoyl)-5-methoxythieno[2,3-b]pyridin-6-yl)oxy)propoxy)-6-methoxyisoindolin-2-yl)-4-oxobutanoate

### Step a: 2,3-dimethoxy-5-vinyl-pyridine

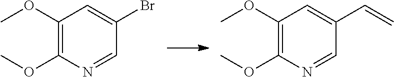

2-ethenyl-4,4,5,5-tetramethyl-1,3,2-dioxaborolane (9.25 g, 60.0595 mmol), Potassium carbonate (18.91 g, 136.8252 mmol) and Pd(dppf)Cl₂ (1.97 g, 2.6923 mmol) was added to a solution of 5-Bromo-2,3-dimethoxypyridine (9.88 g, 45.3112 mmol) in 1,4-Dioxane (100 mL) and Water (20 mL) at 20° C. The reaction mixture was stirred at 80° C. for 2 h under nitrogen atmosphere. The reaction mixture was concentrated and diluted with EA (100 mL), washed with NaHCO₃ (aq)(100 mL) and brine (50 mL). The organics dried over Na₂SO₄, filtered and evaporated under reduced pressure. The residue was purified on silica gel column Ethyl acetate/Heptane (0-20%). The pure fraction was concentrated and dried under vacuo. There was 2,3-dimethoxy-5-vinyl-pyridine (5.22 g, 31.6002 mmol, 69.7404% yield) obtained as a colorless oil. LCMS: (ESI, m/z): [M+H]⁺=166.079. ¹H NMR (400 MHz, DMSO-d₆) δ 7.70 (d, J=1.9 Hz, 1H), 7.46 (t, J=5.8 Hz, 1H), 6.73-6.63 (m, 1H), 5.83 (dd, J=17.6, 0.8 Hz, 1H), 5.24 (dd, J=11.0, 0.8 Hz, 1H), 3.86 (s, 3H), 3.82 (s, 3H).

### Step b: 5,6-dimethoxypyridine-3-carbaldehyde

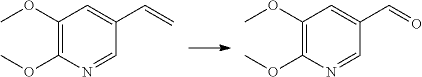

Potassium osmate(VI) dehydrate (0.55 g, 1.4927 mmol) in Water (25 mL) was added to a solution of 2,3-dimethoxy-5-vinyl-pyridine (5.17 g, 31.2975 mmol) and 4-Methylmorpholine N-oxide (7.44 g, 63.5105 mmol) in Tetrahydrofuran (100 mL) at 20° C. The reaction mixture was stirred at 20° C. for 30 min. Sodium periodate (6.82 g, 31.8854 mmol) was added to the mixture at 20° C. The reaction mixture was stirred overnight at 20° C. The reaction mixture was concentrated and diluted with EA (500 mL), washed with water (200 mL) and brine (50 mL). The organics dried over Na₂SO₄, filtered and evaporated under reduced pressure. The residue was purified on silica gel column EA/Hept (0-20%). The pure fraction was concentrated and dried under vacuo. There was 5,6-dimethoxypyridine-3-carbaldehyde (3.51 g, 20.9976 mmol, 67.0904% yield) obtained as a white solid. LCMS: (ESI, m/z): [M+H]⁺=168.058. ¹H NMR (400 MHz, DMSO-d₆) δ 9.95 (s, 1H), 8.34 (d, J=1.9 Hz, 1H), 7.53 (d, J=1.8 Hz, 1H), 3.99 (s, 3H), 3.87 (s, 3H).

### Step c: (5,6-dimethoxy-3-pyridyl)methanol

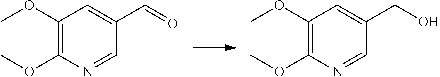

NaBH4 (0.4 g, 10.5729 mmol) was added to a solution of 5,6-dimethoxypyridine-3-carbaldehyde (3.51 g, 20.9976 mmol) in methanol (100 mL) at 20° C. The reaction mixture was stirred at 20° C. for 1 h. The reaction mixture was concentrated and diluted with EA (500 mL), washed with water (200 mL) and brine (200 mL). The organics dried over Na₂SO₄, filtered and evaporated under reduced pressure. The pure fraction was concentrated and dried under vacuo. There was (5,6-dimethoxy-3-pyridyl)methanol (3.33 g, 19.6835 mmol, 93.7418% yield) obtained as a yellow oil. LCMS: (ESI, m/z): [M+H]⁺=170.074. ¹H NMR (400 MHz, DMSO-d₆) δ 7.61 (d, J=1.6 Hz, 1H), 7.23 (t, J=6.4 Hz, 1H), 5.13 (t, J=5.7 Hz, 1H), 4.43 (d, J=5.7 Hz, 2H), 3.84 (s, 3H), 3.77 (s, 3H).

### Step d: (2-bromo-5,6-dimethoxy-3-pyridyl)methanol

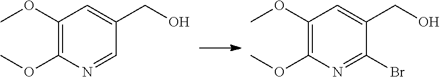

NBS (4.26 g, 23.9347 mmol) and Acetic acid (0.5 mL) was added to a solution of (5,6-dimethoxy-3-pyridyl)methanol (3.30 g, 19.5062 mmol) in Acetonitrile (100 mL) at 20° C. The reaction mixture was stirred at 20° C. for 2 h. The reaction mixture was evaporated under reduced pressure. The resdue was diluted with EA (200 mL), washed with NaHCO₃ (aq)(2×100 mL) and brine (100 mL). The organics dried over Na₂SO₄, filtered and evaporated under reduced pressure. The residue was purified on silica gel column EA/Hept (0-100%). The pure fraction was concentrated and dried under vacuo. There was (2-bromo-5,6-dimethoxy-3-pyridyl)methanol (2.91 g, 11.7304 mmol, 60.1369% yield) obtained as a white solid. LCMS: (ESI, m/z): [M+H]⁺=247.984. ¹H NMR (400 MHz, DMSO-d₆) δ 7.40 (s, 1H), 5.45 (t, J=5.4 Hz, 1H), 4.42 (d, J=5.5 Hz, 2H), 3.85 (s, 3H), 3.80 (s, 3H).

### Step e: 2-bromo-5,6-dimethoxy-pyridine-3-carbaldehyde

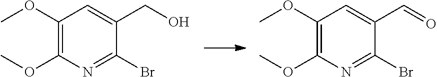

Dess-Martin periodinane (7.3 g, 17.2113 mmol) was added to a solution of (2-bromo-5,6-dimethoxy-3-pyridyl)methanol (2.91 g, 11.7304 mmol) in DCM (100 mL) at 20° C. The reaction mixture was stirred at 20° C. for 1 h. The reaction mixture was evaporated under reduced pressure. The reaction mixture was concentrated and diluted with EA (200 mL), washed with NaHCO₃ (aq)(2×100 mL) and brine (100 mL). The organics dried over Na₂SO₄, filtered and evaporated under reduced pressure. The residue was purified on silica gel column EA/Hept (0-100%). The pure fraction was concentrated and dried under vacuo. There was 2-bromo-5,6-dimethoxy-pyridine-3-carbaldehyde (2.7 g, 10.9730 mmol, 93.5437% yield) obtained as a white solid. LCMS: (ESI, m/z): [M+H]⁺=245.969. H NMR (400 MHz, DMSO-d₆) δ 10.04 (s, 1H), 7.52 (s, 1H), 3.99 (s, 3H), 3.87 (s, 3H).

### Step f: 5,6-dimethoxythieno[2,3-b]pyridine-2-carboxylic acid

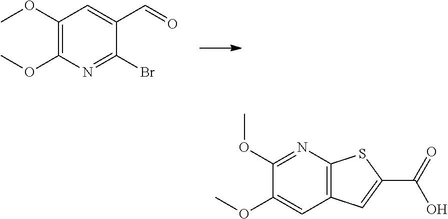 [SMILES: COc1cc(C=O)c(Br)nc1OC ; COc1cc2cc(C(=O)O)sc2nc1OC]

Methyl thioglycolate (1.1870 g, 11.1830 mmol) was added to a mixture of 2-bromo-5,6-dimethoxy-pyridine-3-carbaldehyde (2.68 g, 10.8918 mmol) and Potassium carbonate (4.65 g, 33.6455 mmol) in N,N-Dimethylformamide (50 mL) at 20° C. The reaction mixture was stirred overnight at 60° C. under N₂ atmosphere. The reaction mixture was concentrated and diluted with water (500 mL). The precipitate was collected by filtration, washed with water (100 mL). LiOH (0.40 g, 16.7026 mmol) was added to a solution of the filtrate cake in Tetrahydrofuran (50 mL) and Water (10 mL) at 20° C. The reaction mixture was stirred at 20° C. for 2 h. The reaction mixture was concentrated and diluted with water (200 mL). The mixture was adjusted to pH=3 with HCl (1 M). The precipitate was collected by filtration, washed with water (100 mL). The pure fraction was dried under vacuo. There was 5,6-dimethoxythieno[2,3-b]pyridine-2-carboxylic acid (2.32 g, 9.6971 mmol, 89.0313% yield) obtained as a white solid. LCMS: (ESI, m/z): [M+H]⁺=240.065. ¹H NMR (400 MHz, DMSO-d₆) δ 7.91 (s, 1H), 7.80 (s, 1H), 3.98 (s, 3H), 3.85 (s, 3H).

### Step g: tert-butyl 3-(5,6-dimethoxythieno[2,3-b]pyridin-2-yl)-3-oxo-propanoate

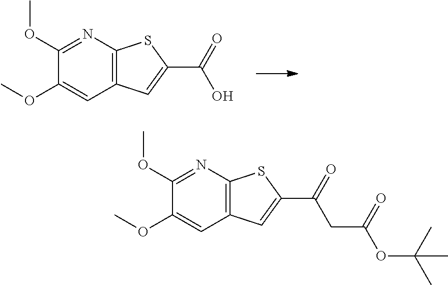

Carbonyl diimidazole (3.11 g, 19.1799 mmol) was added to a solution So?P39 3methoxythieno[2,3-b]pyridine-2-carboxylic acid (2.29 g, 9.5717 mmol) in N,N-Dimethylformamide (50 mL) at 20° C. The reaction mixture was stirred for 30 min at 20° C. under nitrogen atmosphere. 3-tert-Butoxy-3-oxopropanoic acid (3.16 g, 19.7294 mmol), Magnesium chloride (1.87 g, 19.6406 mmol) and Triethylamine (2.96 g, 29.2521 mmol) were added to the mixture at 20° C. The reaction mixture was stirred overnight at 20° C. The reaction mixture was concentrated and diluted with EA (500 mL), washed with NaHCO₃ (aq)(2×300 mL) and brine (200 mL). The organics dried over Na₂SO₄, filtered and evaporated under reduced pressure. The residue was purified on silica gel column EA/n-Hexane (0-50%). The pure fraction was concentrated and dried under vacuo. There was tert-butyl 3-(5,6-dimethoxythieno[2,3-b]pyridin-2-yl)-3-oxo-propanoate (2.37 g, 7.0245 mmol, 73.3885% yield) obtained as a white solid. LCMS: (ESI, m/z): [M+H]⁺=338.098. ¹H NMR (400 MHz, DMSO-d₆) δ 8.15 (d, J=9.8 Hz, 1H), 7.83 (d, J=7.7 Hz, 1H), 4.06 (d, J=8.5 Hz, 2H), 3.98 (s, 3H), 3.88 (s, 3H), 1.41 (s, 9H).

### Step h: 01-tert-butyl 04-ethyl 2-(5,6-dimethoxythieno[2,3-b]pyridine-2-carbonyl)butanedioate

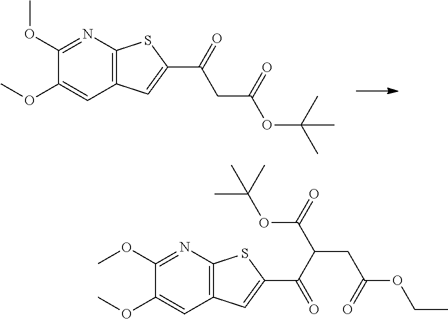

Ethyl bromoacetate (0.63 g, 3.7724 mmol) was added to a mixture of tert-butyl 3-(5,6-dimethoxythieno[2,3-b]pyridin-2-yl)-3-oxo-propanoate (1.2 g, 3.5567 mmol) and Potassium carbonate (0.80 g, 5.7885 mmol) in N,N-Dimethylformamide (20 mL) at 20° C. The reaction mixture was stirred for 6 h at 20° C. The reaction mixture was concentrated and diluted with EA (200 mL), washed with NaHCO₃ (aq)(200 mL) and brine (200 mL). The organics dried over Na₂SO₄, filtered and evaporated under reduced pressure. The residue was purified on silica gel column EA/n-Hexane (0-50%). The pure fraction was concentrated and dried under vacuo. There was 01-tert-butyl 04-ethyl 2-(5,6-dimethoxythieno[2,3-b]pyridine-2-carbonyl)butanedioate (1.72 g, 4.0616 mmol, 114.1951% yield) obtained as a yellow oil. LCMS: (ESI, m/z): [M+H]⁺=424.135. ¹H NMR (400 MHz, DMSO-d₆) δ 8.31 (s, 1H), 7.86 (s, 1H), 4.82 (t, J=7.3 Hz, 1H), 4.05 (m, 2H), 4.00 (s, 3H), 3.87 (s, 3H), 2.92 (d, J=7.2 Hz, 2H), 1.32 (s, 9H), 1.18-1.06 (m, 3H).

### Step i: ethyl 4-(5,6-dimethoxythieno[2,3-b]pyridin-2-yl)-4-oxo-butanoate

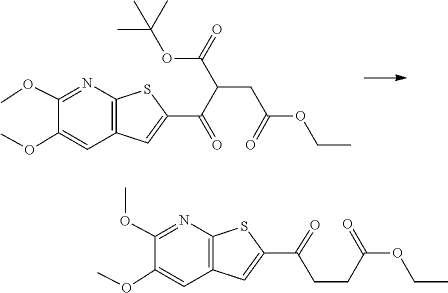

Trifluoroacetic acid (2 mL) was added to a solution of 01-tert-butyl 04-ethyl 2-(5,6-dimethoxythieno[2,3-b]pyridine-2-carbonyl)butanedioate (1.51 g, 3.5657 mmol) in Toluene (10 mL) at 20° C. The reaction mixture was stirred for 1 h at 50° C. The reaction mixture was evaporated under reduced pressure. The residue was purified on C-18 column MeCN/water (0-100%). The pure fraction was concentrated and dried under vacuo. There was ethyl 4-(5,6-dimethoxythieno[2,3-b]pyridin-2-yl)-4-oxo-butanoate (0.94 g, 2.9069 mmol, 81.5252% yield) obtained as a white solid. LCMS: (ESI, m/z): [M+H]⁺=324.083. ¹H NMR (400 MHz, DMSO-d₆) δ 8.20 (s, 1H), 7.79 (s, 1H), 4.00-4.08 (m, 2H), 3.99 (s, 3H), 3.87 (s, 3H), 3.30-3.33 (m, 2H), 2.67 (t, J=6.1 Hz, 2H), 1.18 (t, J=7.0 Hz, 3H).

### Step j: ethyl 4-(6-chloro-5-methoxy-thieno[2,3-b]pyridin-2-yl)-4-oxo-butanoate

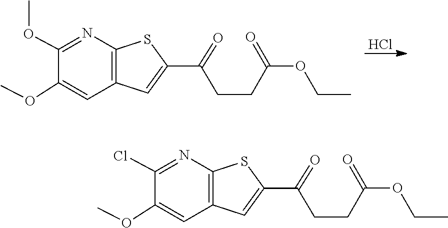

Ethyl 4-(5,6-dimethoxythieno[2,3-b]pyridin-2-yl)-4-oxo-butanoate (0.308 g, 952.4899 mol) was added to HCl (10 mL) at 20° C. The reaction mixture was stirred at 100° C. for 1 h. Cooled the reaction mixture to 20° C., POC₃ (1 mL) was added to the reaction mixture. The reaction mixture was stirred at 100° C. for 2 h under N₂ atmosphere. The reaction mixture was evaporated under reduced pressure. The residue was purified on C-18 column MeCN/water (0-100%). The pure fraction was concentrated and dried under vacuo. There was ethyl 4-(6-chloro-5-methoxy-thieno[2,3-b]pyridin-2-yl)-4-oxo-butanoate (0.25 g, 762.7004 mol, 80.0744% yield) obtained as a white solid. LCMS: (ESI, m/z): [M+H]⁺=328.033. ¹H NMR (400 MHz, DMSO-d₆) δ 8.31 (s, 1H), 8.14 (s, 1H), 4.00-4.08 (m 2H), 3.99 (s, 3H), 3.44-3.36 (m, 2H), 2.70 (t, J=6.2 Hz, 2H), 1.18 (t, J=7.1 Hz, 3H).

### Step k: ethyl 4-(5-(3-((2-(4-ethoxy-4-oxobutanoyl)-5-methoxythieno[2,3-b]pyridin-6-yl)oxy)propoxy)-6-methoxyisoindolin-2-yl)-4-oxobutanoate

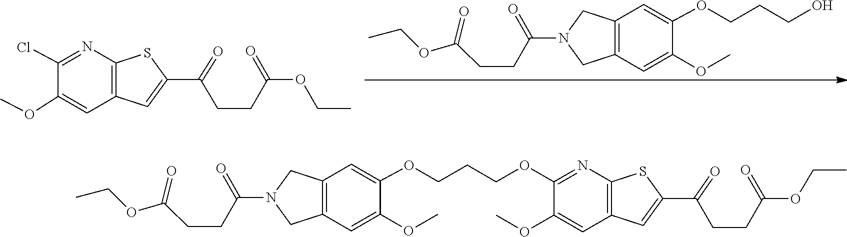 [SMILES: CCOC(=O)CCC(=O)c1cc2cc(OC)c(Cl)nc2s1 ; CCOC(=O)CCC(=O)N1Cc2cc(OC)c(OCCCO)cc2C1 ; CCOC(=O)CCC(=O)c1cc2cc(OC)c(OCCCOc3cc4c(cc3OC)CN(C(=O)CCC(=O)OCC)C4)nc2s1]

RockPhos Palladacycle Gen.3 (0.03 g, 35.7385 mol) was added to a mixture of ethyl 4-(6-chloro-5-methoxy-thieno[2,3-b]pyridin-2-yl)-4-oxo-butanoate (0.10 g, 305.0799 mol), ethyl 4-(5-(3-hydroxypropoxy)-6-methoxyisoindolin-2-yl)-4-oxobutanoate (0.18 g, 512.2470 mol) and Potassium phosphate (0.20 g, 942.2127 μmol) in Toluene (10 mL) at 20° C. The reaction mixture was stirred overnight at 130° C. under N₂ atmosphere. The reaction mixture was evaporated under reduced pressure. The residue was purified on C-18 column MeCN/water (0-100%). The pure fraction was concentrated and dried under vacuo. There was ethyl 4-(5-(3-((2-(4-ethoxy-4-oxobutanoyl)-5-methoxythieno [2,3-b]pyridin-6-yl)oxy)propoxy)-6-methoxyisoindolin-2-yl)-4-oxobutanoate (0.013 g, 20.2267 mol, 6.6300% yield) obtained as a yellow oil. LCMS: (ESI, m/z): [M+H]⁺=643.225.

### Step l: sodium 4-(5-(3-((2-(3-carboxylatopropanoyl)-5-methoxythieno[2,3-b]pyridin-6-yl)oxy)propoxy)-6-methoxyisoindolin-2-yl)-4-oxobutanoate

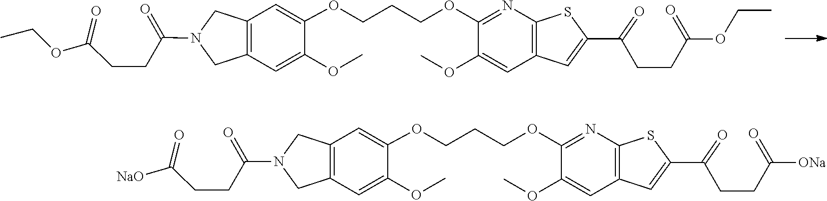

LiOH (3 mg, 125.2699 mol) was added to a solution of ethyl 4-(5-(3-((2-(4-ethoxy-4-oxobutanoyl)-5-methoxythieno[2,3-b]pyridin-6-yl)oxy)propoxy)-6-methoxyisoindolin-2-yl)-4-oxobutanoate (0.018 g, 28.0062 μmol) in Tetrahydrofuran (4 mL) and Water (1 mL) at 20° C. The reaction mixture was stirred at 20° C. for 1 h. The reaction mixture was purified by Prep-HPLC. The pure fraction was concentrated and dried by lyophilization.

NaHCO₃ (3.4 mg, 40.4730 mol) was added to the lyophilized product in Water (5 mL), and stirred at 20° C. for 1 h. The solvent was removed by lyophilization. There was sodium 4-(5-(3-((2-(3-carboxylatopropanoyl)-5-methoxythieno[2,3-b]pyridin-6-yl)oxy)propoxy)-6-methoxyisoindolin-2-yl)-4-oxobutanoate (13 mg, 20.6162 μmol, 73.6129% yield) obtained as a white solid. LCMS: (ESI, m/z): [M+H]⁺=587.162.

### Example II-14 4-(5-(3-((2-(3-carboxypropanoyl)-4-chloro-6-methoxyisoindolin-5-yl)oxy)propoxy)-6-methoxybenzo[b]thiophen-2-yl)-4-oxobutanoic acid

### Step a: ethyl 4-[5-(3-bromopropoxy)-6-methoxy-benzothiophen-2-yl]-4-oxo-butanoate

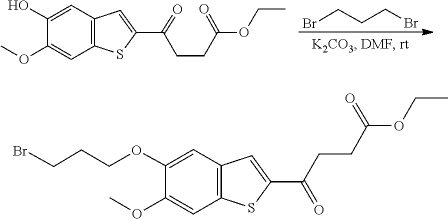

Potassium carbonate (0.113 g, 817.6230 mol) was added to a solution of ethyl 4-(5-hydroxy-6-methoxybenzo[b]thiophen-2-yl)-4-oxobutanoate (0.081 g, 262.6894 mol) and 1,3-dibromopropane (0.263 g, 1.3027 mmol) in DMF (5 mL) at 20° C. under N₂ atmosphere. The reaction mixture was stirred overnight at 20° C. The mixture was purified on C18 column ACN/H₂O (0-60%). The pure fraction was concentrated and dried under vacuo. There was ethyl 4-[5-(3-bromopropoxy)-6-methoxy-benzothiophen-2-yl]-4-oxo-butanoate (0.074 g, 172.3638 mol, 65.6150% yield) obtained as a white solid. LCMS: (ESI, m/z): [M+H]⁺=309.070. ¹H NMR (400 MHz, DMSO-d₆) δ 8.21 (s, 1H), 7.62 (s, 1H), 7.53 (s, 1H), 4.14 (t, J=6.0 Hz, 2H), 4.06 (q, J=7.1 Hz, 2H), 3.87 (s, 3H), 3.70 (t, J=6.5 Hz, 2H), 3.30 (t, J=6.5 Hz, 2H), 2.66 (t, J=6.4 Hz, 2H), 2.35-2.26 (m, 2H), 1.18 (t, J=7.1 Hz, 3H).

### Step b: ethyl 4-(5-(3-((4-chloro-2-(4-ethoxy-4-oxobutanoyl)-6-methoxyisoindolin-5-yl)oxy)propoxy)-6-methoxybenzo[b]thiophen-2-yl)-4-oxobutanoate

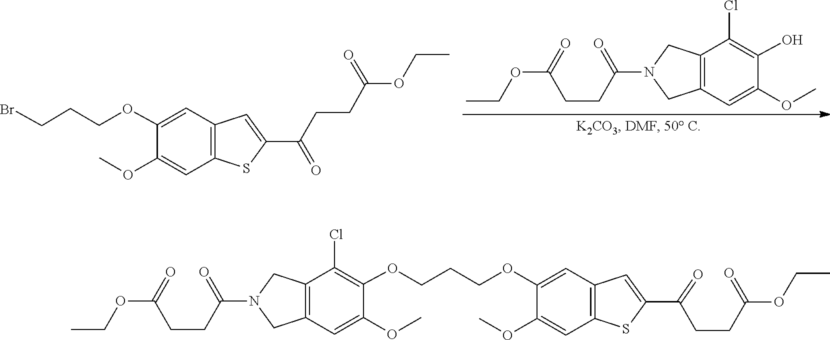 [SMILES: CCOC(=O)CCC(=O)c1cc2cc(OCCCBr)c(OC)cc2s1 ; CCOC(=O)CCC(=O)N1Cc2cc(OC)c(O)c(Cl)c2C1 ; CCOC(=O)CCC(=O)c1cc2cc(OCCCOc3c(OC)cc4c(c3Cl)CN(C(=O)CCC(=O)OCC)C4)c(OC)cc2s1]

Potassium carbonate (0.094 g, 680.1466 mol) was added to a solution of ethyl 4-[5-(3-bromopropoxy)-6-methoxy-benzothiophen-2-yl]-4-oxo-butanoate (0.092 g, 214.2904 mol) and ethyl 4-(4-chloro-5-hydroxy-6-methoxyisoindolin-2-yl)-4-oxobutanoate (0.072 g, 219.6763 mol) in DMF (3 mL) at 20° C. under N₂ atmosphere. The reaction mixture was heated to 50° C. and stirred overnight. The mixture was purified on C18 column ACN/H₂O (0.1% FA)(0-60%). The pure fraction was concentrated and dried under vacuo. There was ethyl 4-(5-(3-((4-chloro-2-(4-ethoxy-4-oxobutanoyl)-6-methoxyisoindolin-5-yl)oxy)propoxy)-6-methoxybenzo [b]thiophen-2-yl)-4-oxobutanoate (0.089 g, 131.6233 mol, 61.4229% yield) obtained as a white solid. LCMS: (ESI, m/z): [M+H]⁺=676.190. ¹H NMR (400 MHz, DMSO-d₆) δ 8.23 (s, 1H), 7.60 (s, 1H), 7.51 (d, J=4.3 Hz, 1H), 7.07 (s, 0.5H), 7.04 (s, 0.5H), 4.83 (s, 1H), 4.75 (s, 1H), 4.61 (s, 1H), 4.50 (s, 1H), 4.26 (s, 2H), 4.15 (t, J=6.0 Hz, 2H), 4.10-4.01 (m, 4H), 3.86 (s, 3H), 3.77 (s, 3H), 3.31-3.27 (m, 2H), 2.70-2.52 (m, 6H), 2.24-2.14 (m, 2H), 1.18 (t, J=7.1 Hz, 6H).

### Step c: 4-(5-(3-((2-(3-carboxypropanoyl)-4-chloro-6-methoxyisoindolin-5-yl)oxy)propoxy)-6-methoxybenzo[b]thiophen-2-yl)-4-oxobutanoic acid

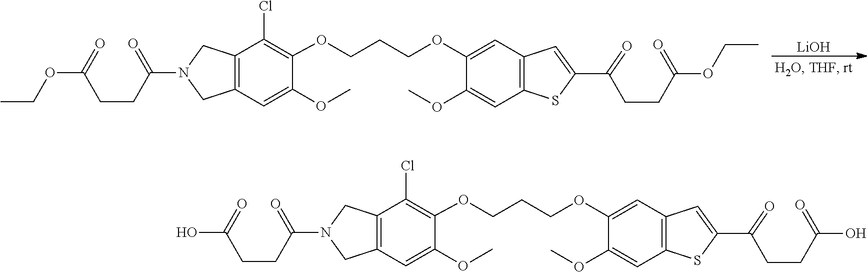

LiOH (0.051 g, 2.1296 mmol) was added to a solution of ethyl 4-(5-(3-((4-chloro-2-(4-ethoxy-4-oxobutanoyl)-6-methoxyisoindolin-5-yl)oxy)propoxy)-6-methoxybenzo[b]thiophen-2-yl)-4-oxobutanoate (0.040 g, 59.1565 mol) in THF (2 mL) and H₂O (2 mL) at 20° C. The reaction mixture was stirred for 2 h at 20° C. The reaction mixture was adjusted to pH=3 with HCl (1 mol/L). The mixture was purified on C18 column ACN/H₂O (0.1% FA)(0-45%). The pure fraction was concentrated and dried by lyophilization. There was 4-(5-(3-((2-(3-carboxypropanoyl)-4-chloro-6-methoxyisoindolin-5-yl)oxy)propoxy)-6-methoxybenzo[b]thiophen-2-yl)-4-oxobutanoic acid (0.025 g, 40.3183 mol, 68.1552% yield) obtained as a white solid. LCMS: (ESI, m/z): [M+H]⁺=620.130. ¹H NMR (400 MHz, DMSO-d₆) δ 8.21 (s, 1H), 7.59 (s, 1H), 7.50 (d, J=3.3 Hz, 1H), 7.06 (s, 0.5H), 7.03 (s, 0.5H), 4.82 (s, 1H), 4.74 (s, 1H), 4.61 (s, 1H), 4.50 (s, 1H), 4.31-4.22 (m, 2H), 4.15 (t, J=6.1 Hz, 2H), 3.86 (s, 3H), 3.77 (s, 3H), 3.26-3.24 (m, 2H), 2.61-2.55 (m, 4H), 2.54-2.44 (m, 2H), 2.19 (t, J=6.2 Hz, 2H).

### Example II-15

### 4-(5-(3-((2-(3-carboxypropanoyl)-5-methoxythieno[3,2-b]pyridin-6-yl)oxy)propoxy)-6-methoxyisoindolin-2-yl)-4-oxobutanoic acid

### Step a: ethyl 4-(5-(3-((2-(4-ethoxy-4-oxobutanoyl)-5-methoxythieno[3,2-b]pyridin-6-yl)oxy)propoxy)-6-methoxyisoindolin-2-yl)-4-oxobutanoate

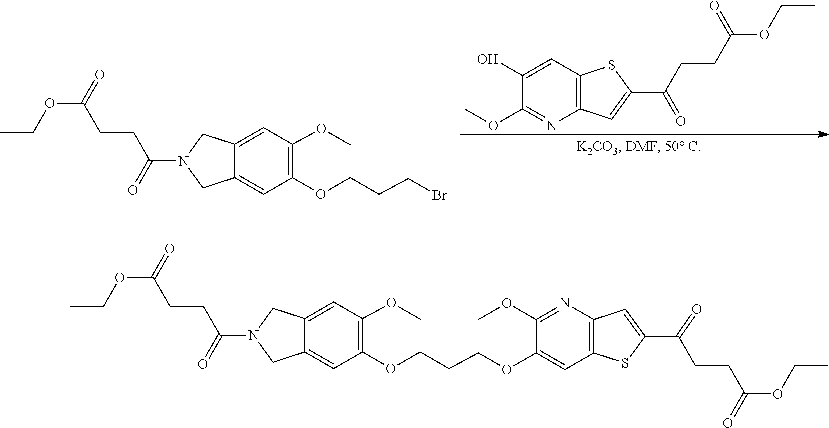 [SMILES: CCOC(=O)CCC(=O)N1Cc2cc(OC)c(OCCCBr)cc2C1 ; CCOC(=O)CCC(=O)c1cc2nc(OC)c(O)cc2s1 ; CCOC(=O)CCC(=O)c1cc2nc(OC)c(OCCCOc3cc4c(cc3OC)CN(C(=O)CCC(=O)OCC)C4)cc2s1]

Potassium carbonate (0.089 g, 643.9686 mol) was added to a solution of ethyl 4-(6-hydroxy-5-methoxythieno[3,2-b]pyridin-2-yl)-4-oxobutanoate (0.069 g, 223.0577 mol) and ethyl 4-(5-(3-bromopropoxy)-6-methoxyisoindolin-2-yl)-4-oxobutanoate (0.079 g, 190.6877 mol) in DMF (2 mL) at 20° C. under N₂ atmosphere. The reaction mixture was heated to 50° C. and stirred overnight. The reaction mixture was purified on C18 column ACN/H₂O (0.1% FA)(0-55%). The pure fraction was concentrated and dried by lyophilization. There was ethyl 4-(5-(3-((2-(4-ethoxy-4-oxobutanoyl)-5-methoxythieno [3,2-b]pyridin-6-yl)oxy)propoxy)-6-methoxyisoindolin-2-yl)-4-oxobutanoate (0.075 g, 116.6925 mol, 61.1956% yield) obtained as a white solid. LCMS: (ESI, m/z): [M+H]⁺=690.130. ¹H NMR (400 MHz, DMSO-d₆) δ 8.27 (s, 1H), 8.03 (s, 1H), 7.06-6.89 (m, 2H), 4.74 (s, 1H), 4.72 (s, 1H), 4.51 (s, 2H), 4.27 (t, J=6.2 Hz, 2H), 4.12 (t, J=6.7 Hz, 2H), 4.09-4.01 (m, 4H), 3.96 (s, 3H), 3.74 (d, J=2.1 Hz, 3H), 3.40-3.34 (m, 2H), 2.68-2.54 (m, 6H), 2.29-2.19 (m, 2H), 1.18 (t, J=7.1 Hz, 6H).

### Step b: 4-(5-(3-((2-(3-carboxypropanoyl)-5-methoxythieno[3,2-b]pyridin-6-yl)oxy)propoxy)-6-methoxyisoindolin-2-yl)-4-oxobutanoic acid

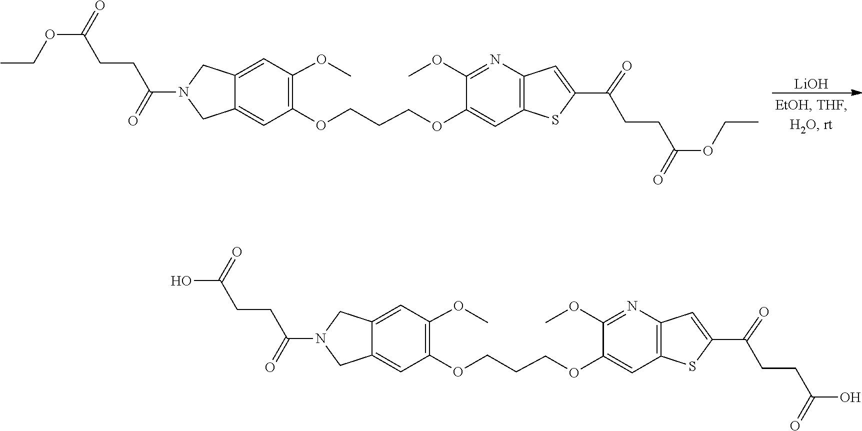

LiOH (0.050 g, 2.0878 mmol) was added to a solution of ethyl 4-(5-(3-((2-(4-ethoxy-4-oxobutanoyl)-5-methoxythieno[3,2-b]pyridin-6-yl)oxy)propoxy)-6-methoxyisoindolin-2-yl)-4-oxobutanoate (0.051 g, 79.3509 μmol) in THF (2 mL), H₂O (2 mL) and Ethanol (1 mL) at 20° C. The reaction mixture was stirred for 2 h at 20° C. The reaction mixture was adjusted to pH=3 with HCl (1 mol/L), The reaction mixture was purified on C18 column ACN/H₂O (0.1% FA)(0-45%). The pure fraction was concentrated and dried by lyophilization. There was 4-(5-(3-((2-(3-carboxypropanoyl)-5-methoxythieno [3,2-b]pyridin-6-yl)oxy)propoxy)-6-methoxyisoindolin-2-yl)-4-oxobutanoic acid (0.015 g, 25.5707 mol, 32.2248% yield) obtained as a white solid. LCMS: (ESI, m/z): [M+H]⁺=586.160. ¹H NMR (400 MHz, DMSO-d₆) δ 8.25 (s, 1H), 8.02 (s, 1H), 7.02-6.92 (m, 2H), 4.73 (s, 1H), 4.71 (s, 1H), 4.51 (s, 2H), 4.27 (t, J=6.2 Hz, 2H), 4.12 (s, J=4.0 Hz, 2H), 3.96 (s, 3H), 3.74 (s, 3H), 3.40-3.34 (m, 2H), 2.63-2.46 (m, 6H), 2.24 (t, J=6.2 Hz, 2H).

### Example II-16

### 4-(5-((2-(((2-(3-carboxypropanoyl)-4-chloro-6-methoxyisoindolin-5-yl)oxy)methyl)allyl)oxy)-4-fluoro-6-methoxybenzo[b]thiophen-2-yl)-4-oxobutanoic acid

### Step a: ethyl 4-[4-chloro-5-[2-(chloromethyl)allyloxy]-6-methoxy-isoindolin-2-yl]-4-oxo-butanoate

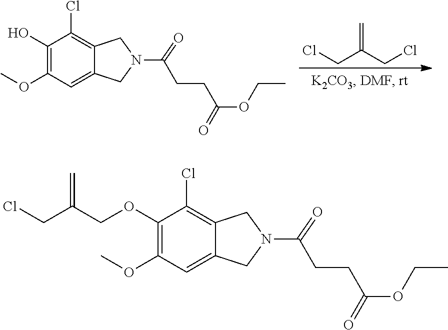

3-Chloro-2-(chloromethyl)prop-1-ene (0.507 g, 2.2635 mmol) was added to a mixture of Potassium carbonate (0.202 g, 1.4616 mmol) and ethyl 4-(4-chloro-5-hydroxy-6-methoxyisoindolin-2-yl)-4-oxobutanoate (0.154 g, 469.8574 mol) in DMF (5 mL) at 20° C. under N₂ atmosphere. The reaction mixture was stirred for 3 h at 20° C. The reaction mixture was purified on C18 column ACN/H₂O (0.1% FA)(0-50%). The pure fraction was concentrated and dried under vacuo. There was ethyl 4-[4-chloro-5-[2-(chloromethyl)allyloxy]-6-methoxy-isoindolin-2-yl]-4-oxo-butanoate (0.151 g, 362.7238 mol, 77.1987% yield) obtained as a white solid. LCMS: (ESI, m/z): [M+H]⁺=416.100.

### Step b: ethyl 4-(5-((2-(((4-chloro-2-(4-ethoxy-4-oxobutanoyl)-6-methoxyisoindolin-5-yl)oxy)methyl)allyl)oxy)-4-fluoro-6-methoxybenzo[b]thiophen-2-yl)-4-oxobutanoate

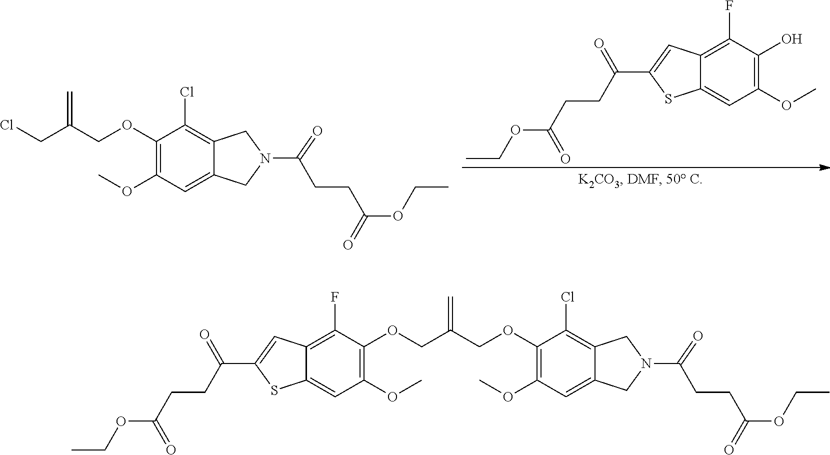

K₂CO₃ (0.092 g, 665.6754 mol) was added to a mixture of ethyl 4-(4-chloro-5-((2-(chloromethyl)allyl)oxy)-6-methoxyisoindolin-2-yl)-4-oxobutanoate (0.068 g, 163.3458 mol) and ethyl 4-(4-fluoro-5-hydroxy-6-methoxybenzo[b]thiophen-2-yl)-4-oxobutanoate (0.049 g, 150.1504 mol) in DMF (3 mL) at 20° C. under N₂ atmosphere. The reaction mixture was stirred overnight at 50° C. The reaction mixture was purified on C18 column ACN/H₂O (0.1% FA)(0-60%). The pure fraction was concentrated and dried by lyophilization. There was ethyl 4-(5-((2-(((4-chloro-2-(4-ethoxy-4-oxobutanoyl)-6-methoxyisoindolin-5-yl)oxy)methyl)allyl)oxy)-4-fluoro-6-methoxybenzo[b]thiophen-2-yl)-4-oxobutanoate (0.022 g, 31.1538 mol, 19.0723% yield) obtained as a white solid. LCMS: (ESI, m/z): [M+H]⁺=706.180. ¹H NMR (400 MHz, DMSO-d₆) δ 8.30 (s, 1H), 7.56 (s, 1H), 7.07 (s, 0.5H), 7.04 (s, 0.5H), 5.34 (d, J=10.9 Hz, 2H), 4.83 (s, 1H), 4.77 (s, 2H), 4.70 (s, 1H), 4.66 (s, 2H), 4.61 (s, 1H), 4.47 (s, 1H), 4.06 (q, J=7.1 Hz, 4H), 3.89 (s, 3H), 3.81 (s, 3H), 3.40-3.34 (m, 2H), 2.69-2.54 (m, 6H), 1.21-1.14 (m, 6H).

### Step c: 4-(5-((2-(((2-(3-carboxypropanoyl)-4-chloro-6-methoxyisoindolin-5-yl)oxy)methyl)allyl)oxy)-4-fluoro-6-methoxybenzo[b]thiophen-2-yl)-4-oxobutanoic acid

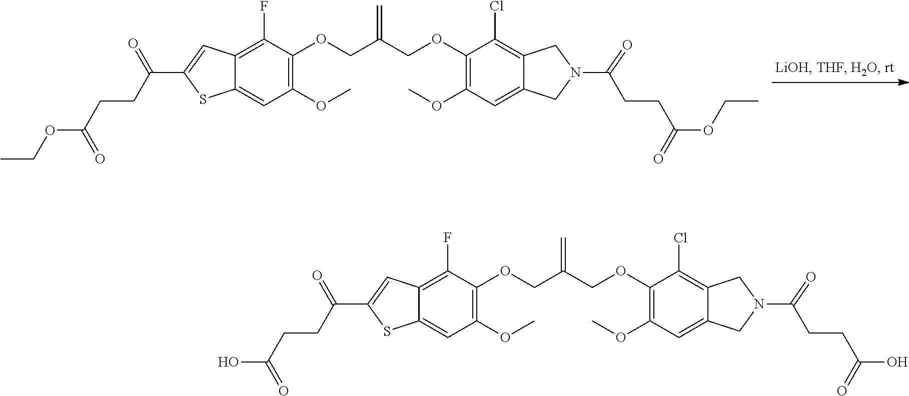 [SMILES: C=C(COc1c(OC)cc2c(c1Cl)CN(C(=O)CCC(=O)OCC)C2)COc1c(OC)cc2sc(C(=O)CCC(=O)OCC)cc2c1F ; C=C(COc1c(OC)cc2c(c1Cl)CN(C(=O)CCC(=O)O)C2)COc1c(OC)cc2sc(C(=O)CCC(=O)O)cc2c1F]

LiOH (0.037 g, 1.5450 mmol) was added to a solution of ethyl 4-(5-((2-(((4-chloro-2-(4-ethoxy-4-oxobutanoyl)-6-methoxyisoindolin-5-yl)oxy)methyl)allyl)oxy)-4-fluoro-6-methoxybenzo [b]thiophen-2-yl)-4-oxobutanoate (0.012 g, 16.9930 mol) in THF (1 mL) and H₂O (1 mL) at 20° C. The reaction mixture was stirred for 1 h at 20° C. The reaction mixture was adjusted to pH=3 with HCl (1 mol/L). The reaction mixture was evaporated under reduced pressure. The residue was purified on C18 column ACN/H₂O (0.1% FA)(0-50%). The pure fraction was concentrated and dried by lyophilization. There was 4-(5-((2-(((2-(3-carboxypropanoyl)-4-chloro-6-methoxyisoindolin-5-yl)oxy)methyl)allyl)oxy)-4-fluoro-6-methoxybenzo[b]thiophen-2-yl)-4-oxobutanoic acid (0.011 g, 16.9213 mol, 99.5782% yield) obtained as a white solid. LCMS: (ESI, m/z): [M−H]20=648.120. ¹H NMR (400 MHz, DMSO-d₆) δ 8.27 (d, J=3.0 Hz, 1H), 7.56 (s, 1H), 7.06 (s, 0.5H), 7.03 (s, 0.5H), 5.34 (d, J=10.8 Hz, 2H), 4.82 (s, 1H), 4.77 (d, J=3.3 Hz, 2H), 4.68 (s, 1H), 4.66 (s, 2H), 4.61 (s, 1H), 4.46 (s, 1H), 3.89 (s, 3H), 3.81 (s, 3H), 3.32-3.26 (m, 2H), 2.61-2.53 (m, 4H), 2.48-2.44 (m, 2H).

### Example II-17 (S)-4-(5-(3-((2-((S)-3-carboxybutanoyl)-4-fluoro-6-methoxybenzo[b]thiophen-5-yl)oxy)propoxy)-4-fluoro-6-methoxyisoindolin-2-yl)-2-methyl-4-oxobutanoic acid

### Step a: 4-fluoro-6-methoxyisoindolin-5-ol hydrochloride

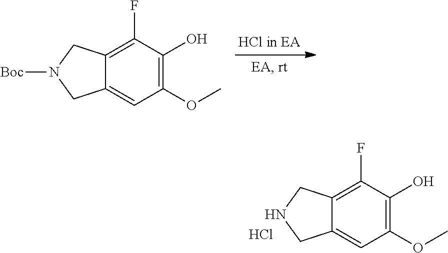 [SMILES: COc1cc2c(c(F)c1O)CN(C(=O)OC(C)(C)C)C2 ; Cl ; COc1cc2c(c(F)c1O)CNC2]

HCl in EA (3 mL, 12 mmol) was added to a solution of tert-butyl 4-fluoro-5-hydroxy-6-methoxyisoindoline-2-carboxylate (0.103 g, 363.5790 mol) in EA (1 mL) at 20° C. The reaction mixture was stirred for 2 h at 20° C.

The reaction mixture was evaporated under reduced pressure. There was 4-fluoro-6-methoxyisoindolin-5-ol hydrochloride (0.12 g, 655.0962 mol, 180.1799% yield) obtained as a white solid, which was used directly to the next step. LCMS: (ESI, m/z): [M+H]⁺=184.070.

### Step b: methyl (2S)-4-(4-fluoro-5-hydroxy-6-methoxy-isoindolin-2-yl)-2-methyl-4-oxo-butanoate

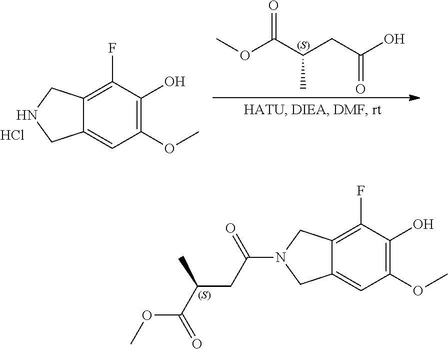

4-fluoro-6-methoxyisoindolin-5-ol hydrochloride (0.112 g, 611.4238 mol) was added to a solution of (S)-4-methoxy-3-methyl-4-oxobutanoic acid (0.108 g, 739.0133 mol), HATU (0.197 g, 518.1084 mol) and DIEA (0.450 g, 3.4818 mmol) in DMF (10 mL) at 20° C. under N₂ atmosphere. The reaction mixture was stirred for 2 h at 20° C. The reaction mixture was purified on C18 column ACN/H₂O (0.1% FA)(0-55%). The pure fraction was concentrated and dried under vacuo. There was methyl (2S)-4-(4-fluoro-5-hydroxy-6-methoxy-isoindolin-2-yl)-2-methyl-4-oxo-butanoate (0.061 g, 195.9494 mol, 32.0481% yield) obtained as a white solid. LCMS: (ESI, m/z): [M+H]⁺=312.120.

### Step c: ethyl (2S)-4-[5-(3-bromopropoxy)-4-fluoro-6-methoxy-benzothiophen-2-yl]-2-methyl-4-oxo-butanoate

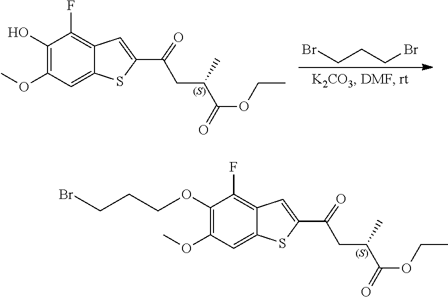

1,3-Dibromopropane (0.255 g, 1.2631 mmol) was added to a mixture of ethyl (S)-4-(4-fluoro-5-hydroxy-6-methoxybenzo[b]thiophen-2-yl)-2-methyl-4-oxobutanoate (0.061 g, 179.2189 mol) and Potassium carbonate (0.129 g, 933.3927 mol) in DMF (3 mL) at 20° C. under N₂ atmosphere. The reaction mixture was stirred for 3 h at 20° C. The mixture was purified on C18 column ACN/H₂O (0.1% FA)(0-70%). The pure fraction was concentrated and dried under vacuo. There was ethyl (2S)-4-[5-(3-bromopropoxy)-4-fluoro-6-methoxy-benzothiophen-2-yl]-2-methyl-4-oxo-butanoate (0.66 g, 1.4306 mmol, 798.2476% yield) obtained as a white solid.

LCMS: (ESI, m/z): [M+H]⁺=461.040.

### Step d: ethyl (S)-4-(4-fluoro-5-(3-((4-fluoro-6-methoxy-2-((S)-4-methoxy-3-methyl-4-oxobutanoyl)isoindolin-5-yl)oxy)propoxy)-6-methoxybenzo[b]thiophen-2-yl)-2-methyl-4-oxobutanoate

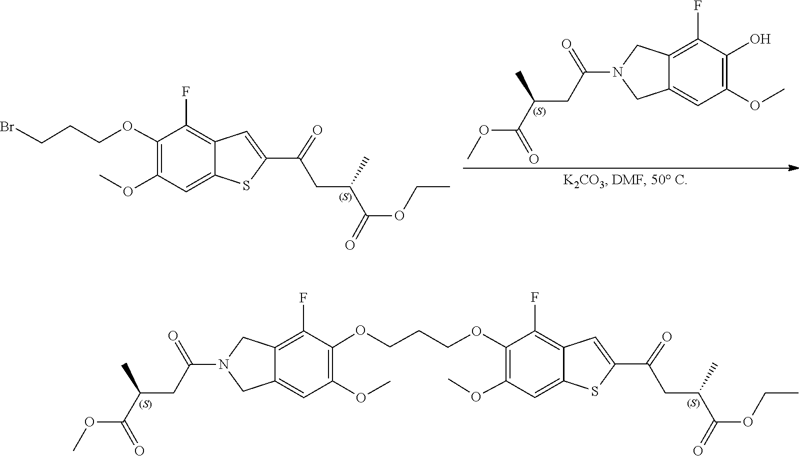

Potassium carbonate (0.074 g, 535.4346 mol) was added to a solution of ethyl (2S)-4-[5-(3-bromopropoxy)-4-fluoro-6-methoxy-benzothiophen-2-yl]-2-methyl-4-oxo-butanoate (0.071 g, 153.8990 mol) and methyl (2S)-4-(4-fluoro-5-hydroxy-6-methoxy-isoindolin-2-yl)-2-methyl-4-oxo-butanoate (0.056 g, 179.8880 mol) in DMF (3 mL) at 20° C. The reaction mixture was stirred overnight at 50° C. under N₂ atmosphere. The reaction mixture was purified on C18 column ACN/H₂O (0.1% FA)(0-50%). The pure fraction was concentrated and dried under vacuo.

There was ethyl (S)-4-(4-fluoro-5-(3-((4-fluoro-6-methoxy-2-((S)-4-methoxy-3-methyl-4-oxobutanoyl)isoindolin-5-yl)oxy)propoxy)-6-methoxybenzo[b]thiophen-2-yl)-2-methyl-4-oxobutanoate (61 mg, 88.1841 mol, 57.3000% yield ) obtained as a white solid. LCMS: (ESI, m/z): [M+H]⁺=692.230. ¹H NMR (400 MHz, DMSO-d₆) δ 8.31 (s, 1H), 7.56 (s, 1H), 6.90 (s, 0.5H), 6.85 (s, 0.5H), 4.77 (s, 2H), 4.56 (s, 1H), 4.54 (s, 1H), 4.25 (t, J=6.1 Hz, 2H), 4.19 (t, J=6.1 Hz, 2H), 4.05 (q, J=7.0 Hz, 2H), 3.88 (s, 3H), 3.77 (d, J=2.1 Hz, 3H), 3.59 (s, 3H), 3.55-3.43 (m, 1H), 3.26-3.15 (m, 1H), 3.00-2.90 (m, 1H), 2.90-2.79 (m, 1H), 2.76-2.66 (m, 1H), 2.50-2.44 (m, 1H), 2.09-1.99 (m, 2H), 1.21-1.10 (m, 9H).

### Step e: (S)-4-(5-(3-((2-((S)-3-carboxybutanoyl)-4-fluoro-6-methoxybenzo[b]thiophen-5-yl)oxy)propoxy)-4-fluoro-6-methoxyisoindolin-2-yl)-2-methyl-4-oxobutanoic acid

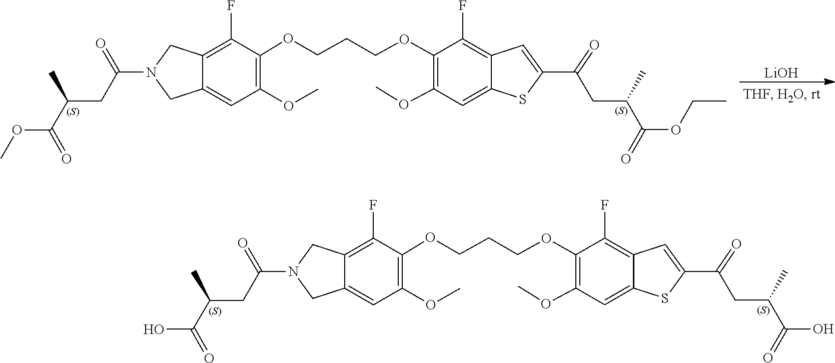

LiOH (0.038 g, 1.5868 mmol) was added to a mixture of ethyl (S)-4-(4-fluoro-5-(3-((4-fluoro-6-methoxy-2-((S)-4-methoxy-3-methyl-4-oxobutanoyl)isoindolin-5-yl)oxy)propoxy)-6-methoxybenzo [b]thiophen-2-yl)-2-methyl-4-oxobutanoate (0.038 g, 54.9344 mol) in THF (2 mL) and H₂O (2 mL) at 20° C. The reaction mixture was stirred for 2 h at 20° C. The reaction mixture was adjusted to pH=3 with HCl (1 mol/L). The reaction mixture was evaporated under reduced pressure. The residue was purified on C18 column ACN/H₂O (0.1% FA)(0-45%).

The pure fraction was concentrated and dried by lyophilization. There was (S)-4-(5-(3-((2-((S)-3-carboxybutanoyl)-4-fluoro-6-methoxybenzo [b]thiophen-5-yl)oxy)propoxy)-4-fluoro-6-methoxyisoindolin-2-yl)-2-methyl-4-oxobutanoic acid (18 mg, 27.7070 mol, 50.4365% yield) obtained as a white solid. LCMS: (ESI, m/z): [M+H]⁺=650.18. ¹H NMR (400 MHz, DMSO-d₆) δ 12.15 (s, 2H), 8.30 (s, 1H), 7.56 (s, 1H), 6.90 (s, 0.5H), 6.85 (s, 0.5H), 4.76 (s, 2H), 4.56 (s, 1H), 4.54 (s, 1H), 4.26 (t, J=6.5 Hz, 2H), 4.19 (t, J=6.1 Hz, 2H), 3.88 (s, 3H), 3.77 (s, 3H), 3.53-3.44 (m, 1H), 3.17-3.05 (m, 1H), 2.93-2.83 (m, 1H), 2.79-2.61 (m, 2H), 2.45-2.35 (m, 1H), 2.11-1.98 (m, 2H), 1.22-1.10 (m, 6H).

### Example II-18

### 4-(5-(3-((2-((2-carboxyethyl)carbamoyl)-6-methoxybenzo[b]thiophen-5-yl)oxy)propoxy)-6-methoxyisoindolin-2-yl)-4-oxobutanoic acid

### Step a: ethyl 3-[[6-methoxy-5-(methoxymethoxy)benzothiophene-2-carbonyl]amino]propanoate

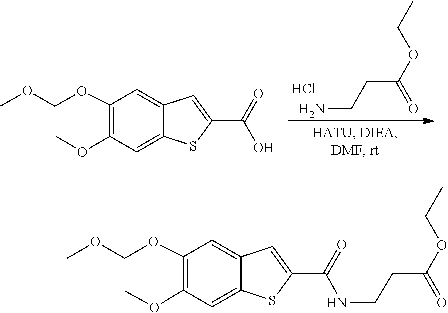

Ethyl 3-aminopropanoate hydrochloride (0.34 g, 2.2134 mmol) was added to a solution of HATU (0.85 g, 2.2355 mmol), DIEA (0.77 g, 5.9578 mmol) and 6-methoxy-5-(methoxymethoxy)benzo[b]thiophene-2-carboxylic acid (0.29 g, 1.0809 mmol) in DMF (15 mL) at 20° C. The reaction mixture was stirred for 2 h at 20° C. The mixture was purified on C18 column ACN/H₂O (0.1% FA)(0-50%). The pure fraction was concentrated and dried under vacuo. There was ethyl 3-[[6-methoxy-5-(methoxymethoxy)benzothiophene-2-carbonyl]amino]propanoate (0.35 g, 952.5987 mol, 88.1269% yield) obtained as a yellow oil. LCMS: (ESI, m/z): [M+H]⁺=368.110.

### Step b: ethyl 3-[(5-hydroxy-6-methoxy-benzothiophene-2-carbonyl)amino]propanoate

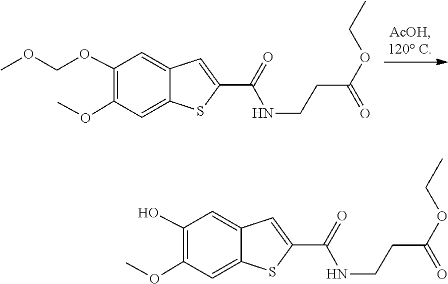

Ethyl 3-[[6-methoxy-5-(methoxymethoxy)benzothiophene-2-carbonyl]amino]propanoate (0.29 g, 789.2963 μmol) was added to Acetic acid (10 mL) at 20° C. The reaction mixture was heated to 120° C. and stirred for 4 h.

The mixture was purified on C18 column ACN/H₂O (0.1% FA)(0-40%). The pure fraction was concentrated and dried under vacuo. There was ethyl 3-[(5-hydroxy-6-methoxy-benzothiophene-2-carbonyl)amino]propanoate (0.23 g, 711.2738 mol, 90.1149% yield) obtained as a white solid. LCMS: (ESI, m/z): [M+H]⁺=324.080.

### Step c: ethyl 4-[5-[3-[2-[(3-ethoxy-3-oxo-propyl)carbamoyl]-6-methoxy-benzothiophen-5-yl]oxypropoxy]-6-methoxy-isoindolin-2-yl]-4-oxo-butanoateethyl

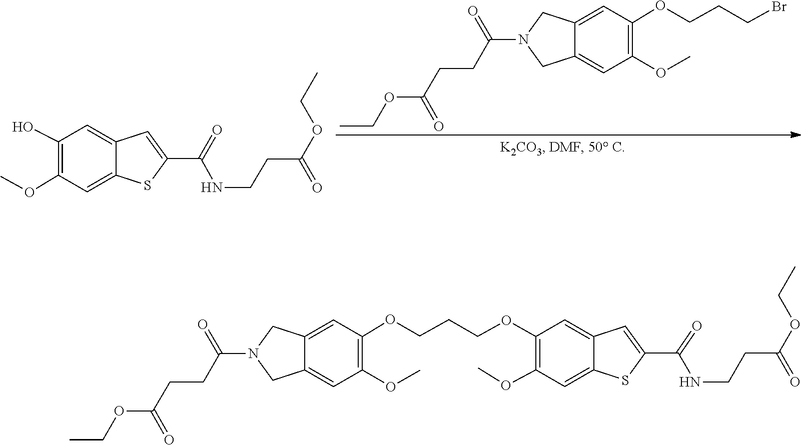

Potassium carbonate (0.25 g, 1.8089 mmol) was added to a solution of ethyl 3-[(5-hydroxy-6-methoxy-benzothiophene-2-carbonyl)amino]propanoate (0.21 g, 649.4249 mol) and ethyl 4-(5-(3-bromopropoxy)-6-methoxyisoindolin-2-yl)-4-oxobutanoate (0.21 g, 506.8913 mol) in DMF (5 mL) at 20° C. under N₂ atmosphere.

The reaction mixture was heated to 50° C. and stirred for 8 h. The reaction mixture was purified on C18 column ACN/H₂O (0.1% FA)(0-65%). The pure fraction was concentrated and dried under vacuo. There was ethyl 4-[5-[3-[2-[(3-ethoxy-3-oxo-propyl)carbamoyl]-6-methoxy-benzothiophen-5-yl]oxypropoxy]-6-methoxy-isoindolin-2-yl]-4-oxo-butanoate (0.147 g, 223.8324 mol, 34.4662% yield) obtained as a white solid. LCMS: (ESI, m/z): [M+H]⁺=657.240.

### Step d: 4-(5-(3-((2-((2-carboxyethyl)carbamoyl)-6-methoxybenzo[b]thiophen-5-yl)oxy)propoxy)-6-methoxyisoindolin-2-yl)-4-oxobutanoic acid

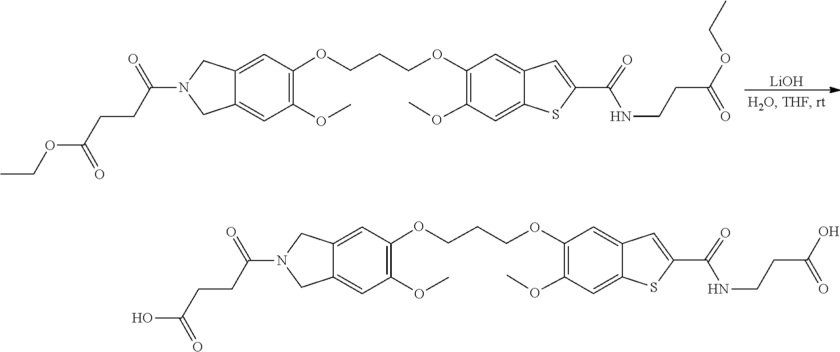

LiOH (0.101 g, 4.2174 mmol) was added to a solution of ethyl 4-[5-[3-[2-[(3-ethoxy-3-oxo-propyl)carbamoyl]-6-methoxy-benzothiophen-5-yl]oxypropoxy]-6-methoxy-isoindolin-2-yl]-4-oxo-butanoate (0.082 g, 124.8589 mol) in THF (8 mL) and H₂O (8 mL) at 20° C. The reaction mixture was stirred for 2 h at 20° C. The reaction mixture was adjusted to pH=3 with HCl (1 mol/L). The mixture was evaporated under reduced pressure. The residue was purified on C18 column ACN/H₂O (0.1% FA)(0-45%). The pure fraction was concentrated and dried by lyophilization. There was 4-(5-(3-((2-((2-carboxyethyl)carbamoyl)-6-methoxybenzo[b]thiophen-5-yl)oxy)propoxy)-6-methoxyisoindolin-2-yl)-4-oxobutanoic acid (49 mg, 81.5803 μmol, 65.3380% yield) obtained as a white solid. LCMS: (ESI, m/z): [M+H]⁺=601.180. ¹H NMR (400 MHz, DMSO-d₆) δ 12.20 (s, 2H), 8.69 (t, J=5.5 Hz, 1H),7.88 (s, 1H), 7.56 (s, 1H), 7.44 (d, J=2.4 Hz, 1H), 7.08-6.90 (m, 2H), 4.74 (s, 1H), 4.71 (s, 1H), 4.53 (s, 2H), 4.25-4.16 (m, 2H), 4.16-4.09 (m, 2H), 3.84 (d, J=1.5 Hz, 3H), 3.74 (d, J=2.1 Hz, 3H), 3.50-3.42 (m, 2H), 2.60-2.52 (m, 4H), 2.50-2.47 (m, 2H), 2.27-2.17 (m, 2H).

### Example II-19

### 4-(5-((2-(((2-(3-carboxypropanoyl)-6-methoxybenzo[b]thiophen-5-yl)oxy)methyl)allyl)oxy)-6-methoxyisoindolin-2-yl)-4-oxobutanoic acid

### Step a: ethyl 4-[5-[2-(chloromethyl)allyloxy]-6-methoxy-isoindolin-2-yl]-4-oxo-butanoate

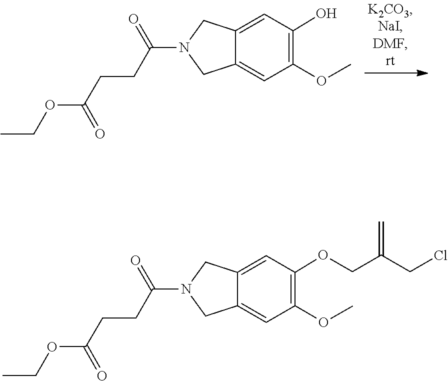

3-Chloro-2-(chloromethyl)prop-1-ene (1.108 g, 8.8643 mmol) was added to a mixture of ethyl 4-(5-hydroxy-6-methoxyisoindolin-2-yl)-4-oxobutanoate (0.507 g, 1.7285 mmol) and Potassium carbonate (0.816 g, 5.9043 mmol) in DMF (20 mL) at 20° C. The reaction mixture was stirred for 8 h at 20° C. The reaction mixture was quenched with adding of water (100 mL) at 20° C., extracted with EA (3×50 mL) and washed with brine (50 mL). The organics dried over Na₂SO₄, filtered and evaporated under reduced pressure. The residue was purified on silica gel column EA/n-Hex (0-70%). The pure fraction was concentrated and dried under vacuo. There was ethyl 4-[5-[2-(chloromethyl)allyloxy]-6-methoxy-isoindolin-2-yl]-4-oxo-butanoate (0.27 g, 707.0847 mol, 40.9069% yield) obtained as a yellow oil. LCMS: (ESI, m/z): [M+H]⁺=382.130.

### Step b: ethyl 4-[5-[2-[[2-(4-ethoxy-4-oxo-butanoyl)-6-methoxy-isoindolin-5-yl]oxymethyl]allyloxy]-6-methoxy-benzothiophen-2-yl]-4-oxo-butanoate

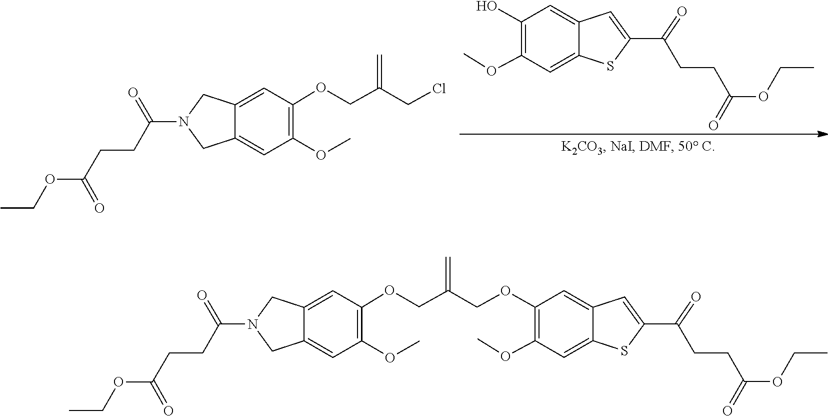

Sodium iodide (0.179 g, 1.1942 mmol) was added to a mixture of ethyl 4-[5-[2-(chloromethyl)allyloxy]-6-methoxy-isoindolin-2-yl]-4-oxo-butanoate (0.216 g, 55.6671 mol),ethyl 4-(5-hydroxy-6-methoxybenzo[b]thiophen-2-yl)-4-oxobutanoate (0.186 g, 603.2128 mol) and Potassium carbonate (0.238 g, 1.7221 mmol) in DMF (5 mL) at 20° C. The reaction mixture was stirred for 5 h at 20° C. under N₂ atmosphere. The mixture was purified on C18 column ACN/H₂O (0.1% FA)(0-70%). The pure fraction was concentrated and dried under vacuo. There was ethyl 4-[5-[2-[[2-(4-ethoxy-4-oxo-butanoyl)-6-methoxy-isoindolin-5-yl]oxymethyl]allyloxy]-6-methoxy-benzothiophen-2-yl]-4-oxo-butanoate (0.169 g, 258.5135 mol, 45.7006% yield) obtained as a white solid. LCMS: (ESI, m/z): [M+H]⁺=654.230. ¹H NMR (400 MHz, DMSO-d₆) δ 8.16 (s, 1H), 7.63 (d, J=4.9 Hz, 1H), 7.53 (s, 1H), 7.05-6.87 (m, 2H), 5.45-5.32 (m, 2H), 4.81-4.72 (m, 3H), 4.66 (s, 3H), 4.52 (s, 1H), 4.50 (s, 1H), 4.10-4.00 (m, 4H), 3.87 (d, J=1.9 Hz, 3H), 3.76 (d, J=2.8 Hz, 3H), 3.32-3.26 (m, 2H), 2.72-2.52 (m, 6H), 1.21-1.13 (m, 6H).

### Step c: 4-(5-((2-(((2-(3-carboxypropanoyl)-6-methoxybenzo[b]thiophen-5-yl)oxy)methyl)allyl)oxy)-6-methoxyisoindolin-2-yl)-4-oxobutanoic acid

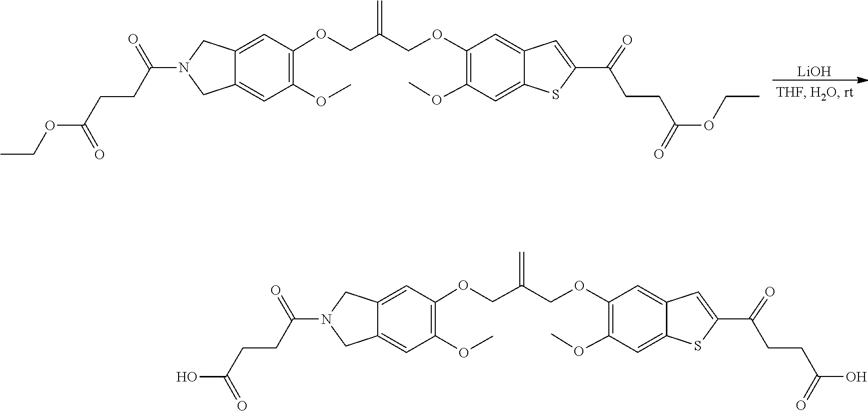

LiOH (0.073 g, 3.0482 mmol) was added to a mixture of ethyl 4-[5-[2-[[2-(4-ethoxy-4-oxo-butanoyl)-6-methoxy-isoindolin-5-yl]oxymethyl]allyloxy]-6-methoxy-benzothiophen-2-yl]-4-oxo-butanoate (0.075 g, 114.7249 mol) in THF (4 mL) and H₂O (2 mL) at 20° C. The reaction mixture was stirred for 2 h at 20° C. The reaction mixture was adjusted to pH=3 with HCl (1 mol/L). The mixture was evaporated under reduced pressure.

The residue was purified on C18 column ACN/H₂O (0.1% FA)(0-45%). The pure fraction was concentrated and dried by lyophilization. There was 4-(5-((2-(((2-(3-carboxypropanoyl)-6-methoxybenzo[b]thiophen-5-yl)oxy)methyl)allyl)oxy)-6-methoxyisoindolin-2-yl)-4-oxobutanoic acid (0.0423 g, 70.7794 mol, 61.6949% yield) obtained as a white solid. LCMS: (ESI, m/z): [M+H]⁺=598.170. ¹H NMR (400 MHz, DMSO-d₆) δ 12.18 (s, 2H), 8.15 (s, 1H), 7.63 (d, J=4.0 Hz, 1H), 7.53 (s, 1H), 7.06-6.90 (m, 2H), 5.45-5.32 (m, 2H), 4.78-4.70 (m, 3H), 4.66 (t, J=3.3 Hz, 3H), 4.53 (s, 1H), 4.50 (s, 1H), 3.87 (d, J=1.6 Hz, 3H), 3.76 (d, J=2.6 Hz, 3H), 3.29-3.24 (m, 2H), 2.65-2.50 (m, 6H).

### Example 11-20

### 4-(5-(3-((2-(3-carboxypropanoyl)-4-fluoro-6-methoxybenzo[b]thiophen-5-yl)oxy)propoxy)-6-methoxyisoindolin-2-yl)-4-oxobutanoic acid

### Step a: ethyl 4-(5-(3-((2-(4-ethoxy-4-oxobutanoyl)-4-fluoro-6-methoxybenzo[b]thiophen-5-yl)oxy)propoxy)-6-methoxyisoindolin-2-yl)-4-oxobutanoate

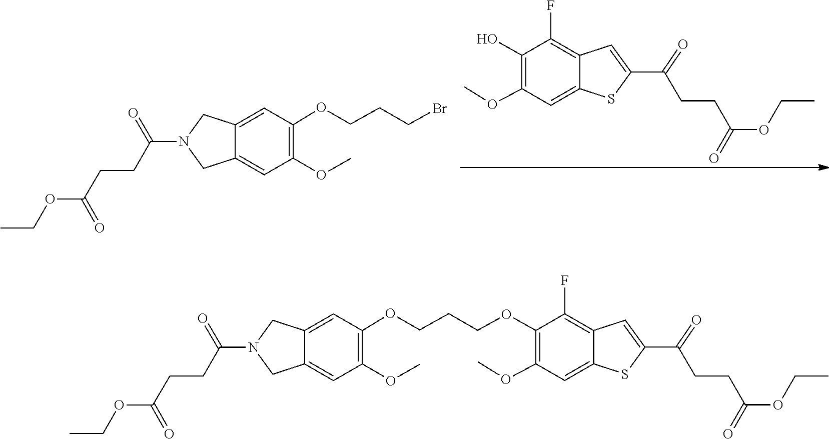

Ethyl 4-(4-fluoro-5-hydroxy-6-methoxybenzo[b]thiophen-2-yl)-4-oxobutanoate (0.081 g, 248.2078 mol) and Potassium carbonate (0.136 g, 984.0419 mol) was added to a solution of ethyl 4-(5-(3-bromopropoxy)-6-methoxyisoindolin-2-yl)-4-oxobutanoate (0.103 g, 248.6182 mol) in N,N-Dimethylformamide (10 mL) at 20° C.

The reaction mixture was stirred overnight at 50° C. The reaction mixture was diluted with Ethyl acetate (100 mL), washed sequentially with water (100 mL) and brine (100 mL). The organic layer was dried over Na₂SO₄, filtered and evaporated under reduced pressure. The residue was purified on C-18 column MeCN/water (0-100%). The pure fraction was concentrated and dried under vacuo. There was ethyl 4-(5-(3-((2-(4-ethoxy-4-oxobutanoyl)-4-fluoro-6-methoxybenzo[b]thiophen-5-yl)oxy)propoxy)-6-methoxyisoindolin-2-yl)-4-oxobutanoate (66 mg, 100.0428 mol, 40.2396% yield) obtained as a white solid. LCMS: (ESI, m/z): [M+H]⁺=432.070. ¹H NMR (400 MHz, DMSO-d₆) δ 8.32 (s, 1H), 7.57 (d, J=3.2 Hz, 1H), 6.98 (s, 0.5H), 6.95 (s, 1H), 6.92 (s, 0.5H), 4.74 (s, 2H), 4.53 (s, 2H), 4.31-4.09 (m, 4H), 4.09-3.98 (m, 4H), 3.87 (s, 3H), 3.73 (s, 3H), 3.41-3.34 (m, 2H), 2.70-2.53 (m, 6H), 2.17-2.04 (m, 2H), 1.21-1.12 (m, 6H).

### Step b: 4-(5-(3-((2-(3-carboxypropanoyl)-4-fluoro-6-methoxybenzo[b]thiophen-5-yl)oxy)propoxy)-6-methoxyisoindolin-2-yl)-4-oxobutanoic acid

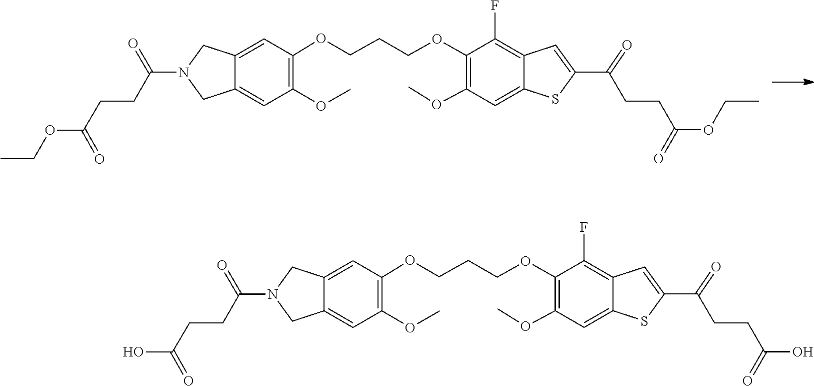 [SMILES: CCOC(=O)CCC(=O)c1cc2c(F)c(OCCCOc3cc4c(cc3OC)CN(C(=O)CCC(=O)OCC)C4)c(OC)cc2s1 ; COc1cc2c(cc1OCCCOc1c(OC)cc3sc(C(=O)CCC(=O)O)cc3c1F)CN(C(=O)CCC(=O)O)C2]

LiOH (0.010 g, 417.5662 mol) was added to a solution of ethyl 4-(5-(3-((2-(4-ethoxy-4-oxobutanoyl)-4-fluoro-6-methoxybenzo[b]thiophen-5-yl)oxy)propoxy)-6-methoxyisoindolin-2-yl)-4-oxobutanoate (0.052 g, 78.8217 mol) in Tetrahydrofuran (10 mL) and Water (2 mL). The reaction mixture was stirred for 2 hours at 50° C. The reaction mixture was acidified pH=4 with HCl (1 M). The mixture was evaporated under reduced pressure. The residue was purified on C-18 column MeCN/water (0-100%). The pure fraction was concentrated and dried under vacuo. There was 4-(5-(3-((2-(3-carboxypropanoyl)-4-fluoro-6-methoxybenzo[b]thiophen-5-yl)oxy)propoxy)-6-methoxyisoindolin-2-yl)-4-oxobutanoic acid (22 mg, 36.4473 mol, 46.2402% yield) obtained as a white solid. LCMS: (ESI, m/z): [M+H]⁺=604.157. ¹H NMR (400 MHz, DMSO-d₆) δ 12.15 (s, 2H), 8.30 (s, 1H), 7.57 (s, 1H), 6.98 (s, 0.5H), 6.95 (s, 1H), 6.92 (s, 0.5H), 4.74 (s, 2H), 4.53 (s, 2H), 4.33-4.06 (m, 4H), 3.91 (s, 3H), 3.68 (s, 3H), 3.30-3.38 (m, 2H), 2.71-2.53 (m, 6H), 2.18-2.10 (m, 2H).

### Example II-21

### Trans-2-(5-(3-((2-(3-carboxypropanoyl)-4-fluoro-6-methoxyisoindolin-5-yl)oxy)propoxy)-6-methoxybenzo[b]thiophene-2-carbonyl)cyclopropane-1-carboxylic acid

### Step a: methyl trans-2-[5-(5,5-dimethyl-1,3,2-dioxaborinan-2-yl)-6-methoxy-benzothiophene-2-carbonyl]cyclopropanecarboxylate

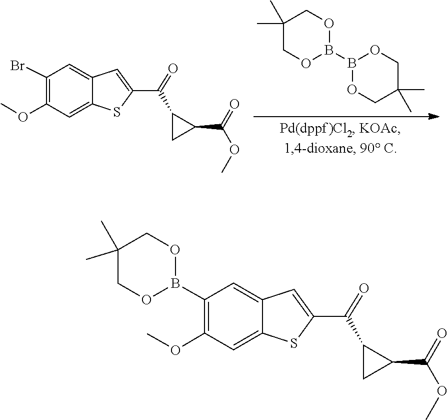

[1,1′-Bis(diphenylphosphino)ferrocene]dichloropalladium(II) (0.203 g, 277.4347 mol) was added to a mixture of methyl trans-2-(5-bromo-6-methoxybenzo[b]thiophene-2-carbonyl)cyclopropane-1-carboxylate (0.510 g, 1.3813 mmol), bis(neopentyl glycolato)diboron (0.953 g, 4.2190 mmol) and KOAc (0.437 g, 4.4527 mmol) in 1,4-Dioxane (20 mL) at 20° C. The reaction mixture was heated to 90° C. and stirred overnight under N₂ atmosphere. The reaction mixture was filtered through a celite pad. The filtrate was evaporated under reduced pressure. There was a crude methyl trans-2-[5-(5,5-dimethyl-1,3,2-dioxaborinan-2-yl)-6-methoxy-benzothiophene-2-carbonyl]cyclopropanecarboxylate (1.61 g, 2.0012 mmol, 144.8795% yield) obtained as a black oil, which was directly used in the next step. LCMS: (ESI, m/z): [M+H]⁺=403.13.

### Step b: methyl trans-2-(5-hydroxy-6-methoxy-benzothiophene-2-carbonyl)cyclopropanecarboxylate

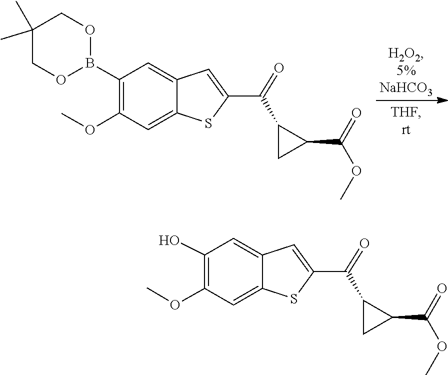 [SMILES: COC(=O)[C@H]1C[C@@H]1C(=O)c1cc2cc(B3OCC(C)(C)CO3)c(OC)cc2s1 ; COC(=O)[C@H]1C[C@@H]1C(=O)c1cc2cc(O)c(OC)cc2s1]

H₂O₂(3.0 mL, 24.7468 mmol, 30% aqueous solution) was added to a mixture of methyl trans-2-[5-(5,5-dimethyl-1,3,2-dioxaborinan-2-yl)-6-methoxy-benzothiophene-2-carbonyl]cyclopropanecarboxylate (1.56 g, 1.9390 mmol) and NaHCO₃ (3.0 mL, 1.6722 mmol, 5% aqueous solution) in THF (30 mL) at 20° C. The reaction mixture was stirred for 1 h at 20° C. The resulting reaction mixture was diluted with brine (150 mL), extracted with EA (150 mL) and washed with brine (150 mL). The organic layer was collected and dried over Na₂SO₄, filtered and evaporated under reduced pressure. The residue was purified by silica gel column chromatography, eluting with ethyl acetate/n-hexane (0-100%). The pure fraction was concentrated and dried under reduced pressure. There was methyl trans-2-(5-hydroxy-6-methoxy-benzothiophene-2-carbonyl)cyclopropanecarboxylate (0.30 g, 979.3261 mol, 71.43% yield) obtained as a reddish brown solid. LCMS: (ESI, m/z): [M+H]⁺=307.05.

¹H NMR (400 MHz, CDCl₃-d) 6 7.98 (s, 1H), 7.38 (s, 1H), 7.26 (s, 1H), 4.02 (s, 3H), 3.76 (s, 3H), 3.23-3.11 (m, 1H), 2.49-2.38 (m, 1H), 1.73-1.66 (m, 1H), 1.65-1.58 (m, 1H).

### Step c: tert-butyl 4-fluoro-6-methoxy-5-[3-[6-methoxy-2-[trans-2-methoxycarbonylcyclopropanecarbonyl]benzothiophen-5-yl]oxypropoxy] isoindoline-2-carboxylate

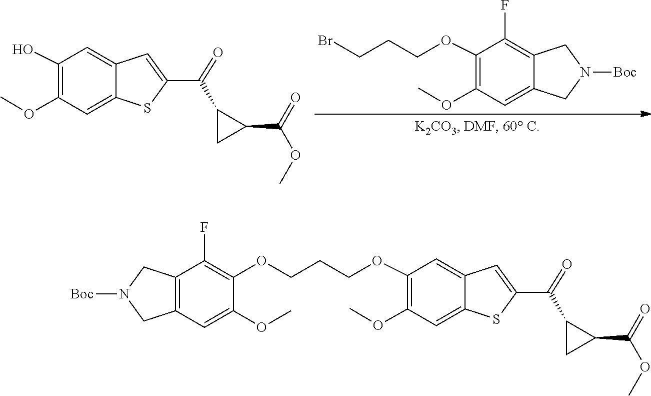 [SMILES: COC(=O)[C@H]1C[C@@H]1C(=O)c1cc2cc(O)c(OC)cc2s1 ; COc1cc2c(c(F)c1OCCCBr)CN(C(=O)OC(C)(C)C)C2 ; COC(=O)[C@H]1C[C@@H]1C(=O)c1cc2cc(OCCCOc3c(OC)cc4c(c3F)CN(C(=O)OC(C)(C)C)C4)c(OC)cc2s1]

K₂CO₃ (0.501 g, 3.6250 mmol) was added to a solution of methyl trans-2-(5-hydroxy-6-methoxy-benzothiophene-2-carbonyl)cyclopropanecarboxylate (0.300 g, 979.3264 mol) and tert-butyl 5-(3-bromopropoxy)-4-fluoro-6-methoxyisoindoline-2-carboxylate (0.406 g, 1.0043 mmol) in DMF (10 mL) at 20° C. The reaction mixture was heated to 60° C. and stirred overnight. The resulting reaction mixture was diluted with H₂O (150 mL), extracted with EA (150 mL) and washed with brine (2×150 mL). The organic layer was collected and dried over Na₂SO₄, filtered and evaporated under reduced pressure. The residue was purified by silica gel column chromatography, eluting with ethyl acetate/n-hexane (0-50%). The pure fraction was concentrated and dried under reduced pressure. There was tert-butyl 4-fluoro-6-methoxy-5-[3-[6-methoxy-2-[trans-2-methoxycarbonylcyclopropanecarbonyl]benzothiophen-5-yl]oxypropoxy] isoindoline-2-carboxylate (0.45 g, 714.6357 mol, 72.9722% yield) obtained as a yellowish solid. LCMS: (ESI, m/z): [M+H-Boc]⁺=530.20. ¹H NMR (400 MHz, CDCl₃-d) 6 7.92 (s, 1H), 7.29 (s, 1H), 7.18 (s, 1H), 6.50 (s, 1H), 4.55 (s, 4H), 4.28 (t, J=6.4 Hz, 2H), 4.20 (t, J=5.8 Hz, 2H), 3.87 (s, 3H), 3.71 (s, 3H), 3.67 (s, 3H), 3.11-3.04 (m, 1H), 2.38-2.31 (m, 1H), 2.30-2.20 (m, 2H), 1.65-1.58 (m, 1H), 1.57-1.50 (m, 1H), 1.44 (s, 9H).

### Step d: methyl trans-2-[5-[3-(4-fluoro-6-methoxy-isoindolin-5-yl)oxypropoxy]-6-methoxy-benzothiophene-2-carbonyl]cyclopropanecarboxylate

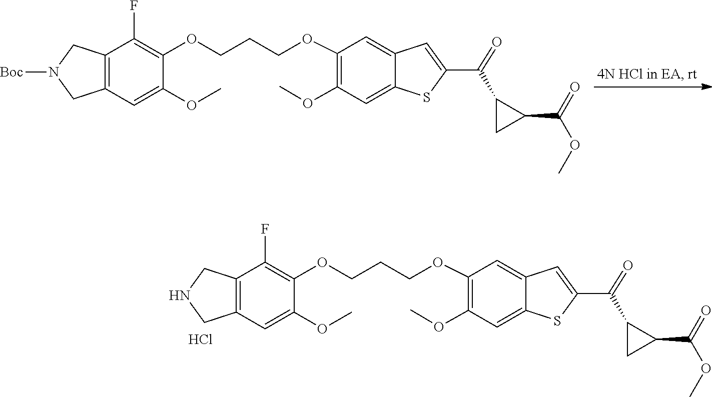 [SMILES: COC(=O)[C@H]1C[C@@H]1C(=O)c1cc2cc(OCCCOc3c(OC)cc4c(c3F)CN(C(=O)OC(C)(C)C)C4)c(OC)cc2s1 ; COC(=O)[C@H]1C[C@@H]1C(=O)c1cc2cc(OCCCOc3c(OC)cc4c(c3F)CNC4)c(OC)cc2s1 ; Cl]

HCl (4 N) in EA (15 mL, 60 mmol) was added to a solution of tert-butyl 4-fluoro-6-methoxy-5-[3-[6-methoxy-2-[trans-2-methoxycarbonylcyclopropanecarbonyl]benzothiophen-5-yl]oxypropoxy] isoindoline-2-carboxylate (0.45 g, 714.6351 mol) in EA (5 mL) at 20° C. The reaction mixture was stirred for 2 h at 20° C. The resulting reaction mixture was concentrated under reduced pressure. There was methyl trans-2-[5-[3-(4-fluoro-6-methoxy-isoindolin-5-yl)oxypropoxy]-6-methoxy-benzothiophene-2-carbonyl]cyclopropanecarboxylate (0.42 g, 667.8009 mol, 93.4464% yield, HCl salt) obtained as a gray solid. LCMS: (ESI, m/z): [M+H]⁺=530.20. ¹H NMR (400 MHz, DMSO-d₆) δ 10.17 (s, 2H), 8.49 (s, 1H), 7.63 (s, 1H), 7.52 (s, 1H), 6.98 (s, 1H), 4.50 (s, 2H), 4.46 (s, 2H), 4.23 (t, J=6.0 Hz, 2H), 4.17 (t, J=5.8 Hz, 2H), 3.86 (s, 3H), 3.77 (s, 3H), 3.68 (s, 3H), 3.30-3.26 (m, 1H), 2.27-2.20 (m, 1H), 2.19-2.11 (m, 2H), 1.54 (t, J=7.1 Hz, 2H).

### Step e: methyl trans-2-[5-[3-[2-(4-ethoxy-4-oxo-butanoyl)-4-fluoro-6-methoxy-isoindolin-5-yl]oxypropoxy]-6-methoxy-benzothiophene-2-carbonyl]cyclopropanecarboxylate

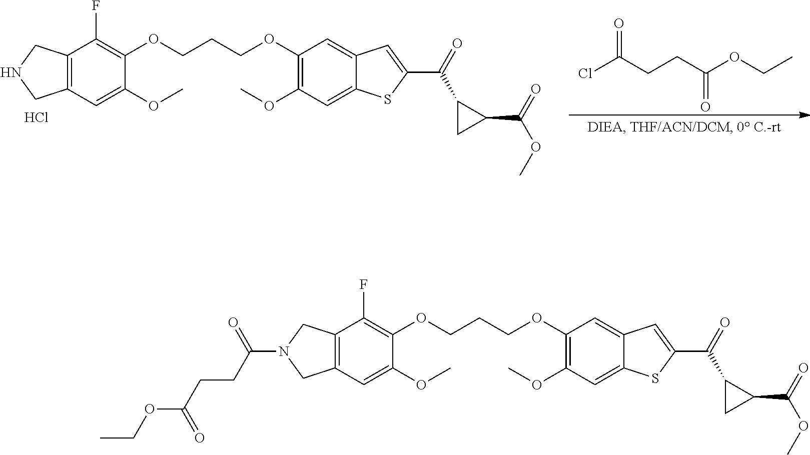

Ethyl 4-chloro-4-oxobutanoate (0.055 mL, 385.9673 mol) in DCM (2 mL) was added dropwise to a solution of methyl trans-2-[5-[3-(4-fluoro-6-methoxy-isoindolin-5-yl)oxypropoxy]-6-methoxy-benzothiophene-2-carbonyl]cyclopropanecarboxylate (0.202 g, 356.8671 mol, HCl salt) and N,N-Diisopropylethylamine (0.185 mL, 1.0621 mmol) in THF (4 mL) and ACN (4 mL) at 0° C. The reaction mixture was stirred for 2 h at 20° C. The reaction mixture was concentrated under reduced pressure. The residue was purified by Prep-TLC with the developing solvent of DCM/MeOH (25:1). There was methyl trans-2-[5-[3-[2-(4-ethoxy-4-oxo-butanoyl)-4-fluoro-6-methoxy-isoindolin-5-yl]oxypropoxy]-6-methoxy-benzothiophene-2-carbonyl]cyclopropanecarboxylate (0.221 g, 336.0184 mol, 94.1579% yield) obtained as an off-white semi-solid. LCMS: (ESI, m/z): [M+H]⁺=658.25. ¹H NMR (400 MHz, CDCl₃-d) 6 7.93 (s, 1H), 7.19 (s, 2H), 6.54 (s, 0.5H), 6.49 (s, 0.5H), 4.74 (d, J=7.7 Hz, 2H), 4.68 (d, J=5.6 Hz, 2H), 4.29 (s, 2H), 4.21 (s, 2H), 4.09 (q, J=7.1 Hz, 2H), 3.88 (s, 3H), 3.73 (s, 3H), 3.68 (s, 3H), 3.15-3.02 (m, 1H), 2.72-2.54 (m, 4H), 2.40-2.31 (m, 1H), 2.30-2.18 (m, 2H), 1.65-1.51 (m, 2H), 1.20 (t, J=7.0 Hz, 3H).

### Step f: trans-2-(5-(3-((2-(3-carboxypropanoyl)-4-fluoro-6-methoxyisoindolin-5-yl)oxy)propoxy)-6-methoxybenzo[b]thiophene-2-carbonyl)cyclopropane-1-carboxylic acid

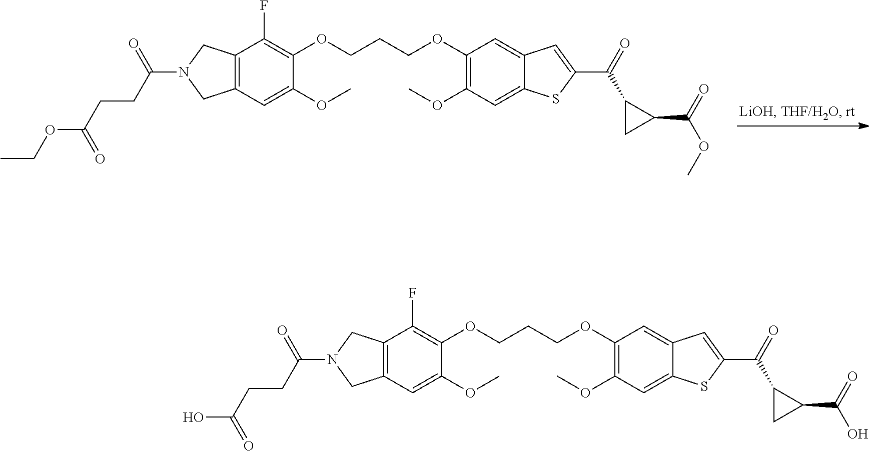 [SMILES: CCOC(=O)CCC(=O)N1Cc2cc(OC)c(OCCCOc3cc4cc(C(=O)[C@H]5C[C@@H]5C(=O)OC)sc4cc3OC)c(F)c2C1 ; COc1cc2sc(C(=O)[C@H]3C[C@@H]3C(=O)O)cc2cc1OCCCOc1c(OC)cc2c(c1F)CN(C(=O)CCC(=O)O)C2]

LiOH (0.027 g, 1.1274 mmol) was added to a solution of methyl trans-2-[5-[3-[2-(4-ethoxy-4-oxo-butanoyl)-4-fluoro-6-methoxy-isoindolin-5-yl]oxypropoxy]-6-methoxy-benzothiophene-2-carbonyl]cyclopropanecarboxylate (0.165 g, 250.8735 mol) in THF (6 mL) and H₂O (2 mL) at 20° C. The reaction mixture was stirred for 2 h at 20° C. The resulting reaction mixture was adjusted to pH=2-3 with HCl (6 N) and evaporated under reduced pressure. The residue was purified by reverse C18 column chromatography, eluting with the mobilie phase ACN/H₂O (0.1% FA)(0-100%). The pure fraction was concentrated and lyophilized overnight. There was trans-2-(5-(3-((2-(3-carboxypropanoyl)-4-fluoro-6-methoxyisoindolin-5-yl)oxy)propoxy)-6-methoxybenzo[b]thiophene-2-carbonyl)cyclopropane-1-carboxylic acid (0.052 g, 84.4674 mol, 33.6693% yield) obtained as a white solid. LCMS: (ESI, m/z): [M+H]⁺=616.20. ¹H NMR (400 MHz, DMSO-d₆) δ 12.45 (s, 2H), 8.48 (s, 1H), 7.61 (s, 1H), 7.51 (d, J=2.2 Hz, 1H), 6.91 (s, 0.5H), 6.88 (s, 0.5H), 4.78 (d, J=5.5 Hz, 2H), 4.56 (s, 2H), 4.23 (t, J=5.0 Hz, 2H), 4.17 (t, J=5.9 Hz, 2H), 3.86 (s, 3H), 3.76 (s, 3H), 3.27-3.19 (m, 1H), 2.60-2.53 (m, 2H), 2.49-2.44 (m, 2H), 2.21-2.07 (m, 3H), 1.49 (t, J=7.1 Hz, 2H).

The compounds of the following example shown in table 4 were synthesized using the above procedure with the corresponding starting materials.

TABLE 4

| | | MS:(M + |
|---|---|---|
| EX No. | | H)⁺ & |
| II- | Structure | ¹HNMR |
| 22. | 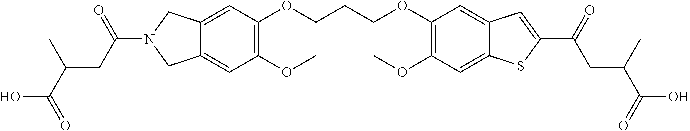 [SMILES: COc1cc2c(cc1OCCCOc1cc3cc(C(=O)CC(C)C(=O)O)sc3cc1OC)CN(C(=O)CC(C)C(=O)O)C2] | 614 |
| 23. | 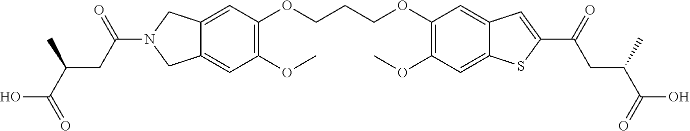 [SMILES: COc1cc2c(cc1OCCCOc1cc3cc(C(=O)C[C@H](C)C(=O)O)sc3cc1OC)CN(C(=O)C[C@H](C)C(=O)O)C2] | 614 |
| 24. | 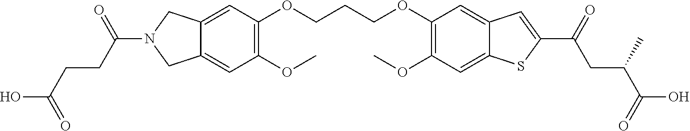 [SMILES: COc1cc2c(cc1OCCCOc1cc3cc(C(=O)C[C@H](C)C(=O)O)sc3cc1OC)CN(C(=O)CCC(=O)O)C2] | 600 |
| 25. | 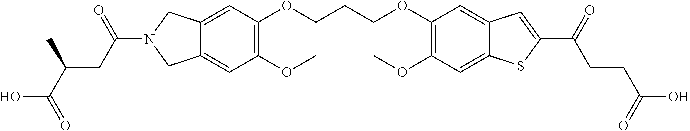 [SMILES: COc1cc2c(cc1OCCCOc1cc3cc(C(=O)CCC(=O)O)sc3cc1OC)CN(C(=O)C[C@H](C)C(=O)O)C2] | 600 |
| 26. | 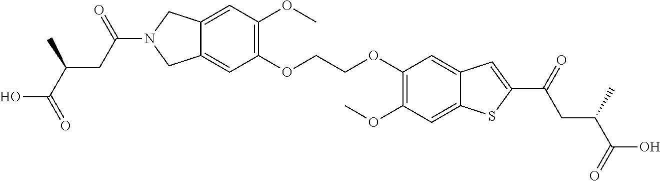 [SMILES: COc1cc2c(cc1OCCOc1cc3cc(C(=O)C[C@H](C)C(=O)O)sc3cc1OC)CN(C(=O)C[C@H](C)C(=O)O)C2] | 600 |
| 27. | 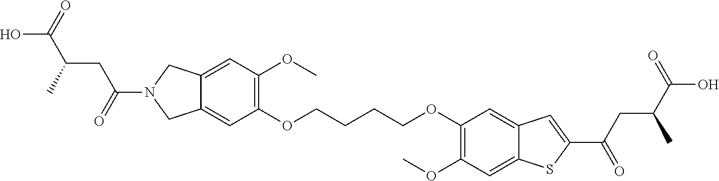 [SMILES: COc1cc2c(cc1OCCCCOc1cc3cc(C(=O)C[C@H](C)C(=O)O)sc3cc1OC)CN(C(=O)C[C@H](C)C(=O)O)C2] | 628 |
| 28. | 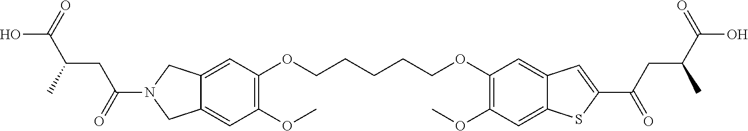 [SMILES: COc1cc2c(cc1OCCCCCOc1cc3cc(C(=O)C[C@H](C)C(=O)O)sc3cc1OC)CN(C(=O)C[C@H](C)C(=O)O)C2] | 642 |
| 29. | 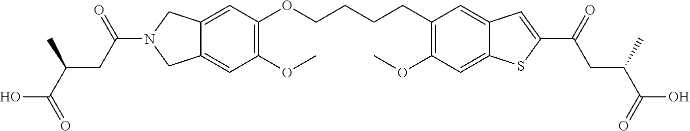 [SMILES: COc1cc2sc(C(=O)C[C@H](C)C(=O)O)cc2cc1CCCCOc1cc2c(cc1OC)CN(C(=O)C[C@H](C)C(=O)O)C2] | 612 |
| 30. | 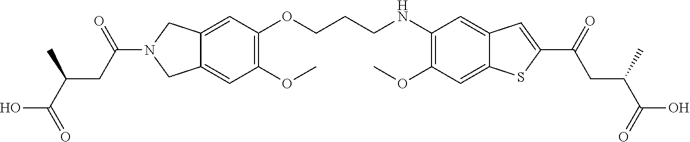 [SMILES: COc1cc2sc(C(=O)C[C@H](C)C(=O)O)cc2cc1NCCCOc1cc2c(cc1OC)CN(C(=O)C[C@H](C)C(=O)O)C2] | 613 |
| 31. | 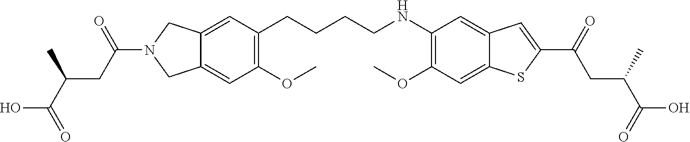 [SMILES: COc1cc2c(cc1CCCCNc1cc3cc(C(=O)C[C@H](C)C(=O)O)sc3cc1OC)CN(C(=O)C[C@H](C)C(=O)O)C2] | 611 |
| 32. | 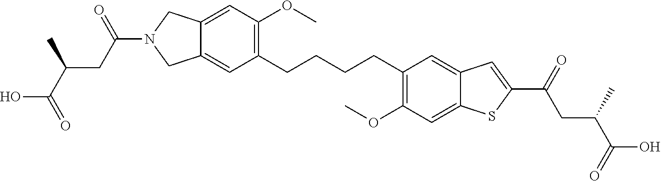 [SMILES: COc1cc2c(cc1CCCCc1cc3cc(C(=O)C[C@H](C)C(=O)O)sc3cc1OC)CN(C(=O)C[C@H](C)C(=O)O)C2] | 596 |
| 33. | 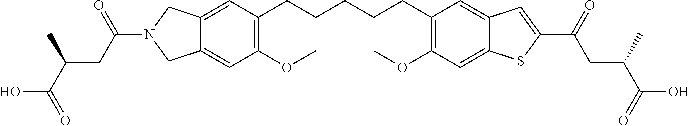 [SMILES: COc1cc2c(cc1CCCCCc1cc3cc(C(=O)C[C@H](C)C(=O)O)sc3cc1OC)CN(C(=O)C[C@H](C)C(=O)O)C2] | 610 |
| 34. | 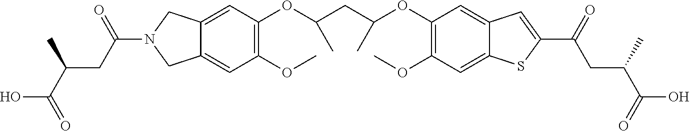 [SMILES: COc1cc2c(cc1OC(C)CC(C)Oc1cc3cc(C(=O)C[C@H](C)C(=O)O)sc3cc1OC)CN(C(=O)C[C@H](C)C(=O)O)C2] | 642 |
| 35. | 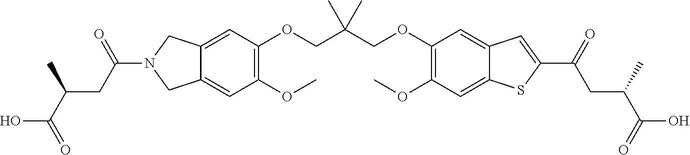 [SMILES: COc1cc2c(cc1OCC(C)(C)COc1cc3cc(C(=O)C[C@H](C)C(=O)O)sc3cc1OC)CN(C(=O)C[C@H](C)C(=O)O)C2] | 642 |
| 36. | 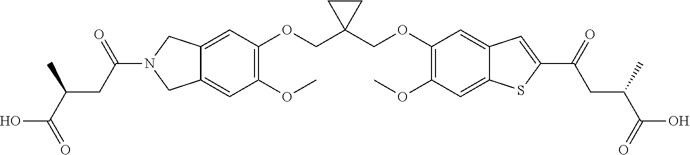 [SMILES: COc1cc2c(cc1OCC1(COc3cc4cc(C(=O)C[C@H](C)C(=O)O)sc4cc3OC)CC1)CN(C(=O)C[C@H](C)C(=O)O)C2] | 640 |
| 37. | 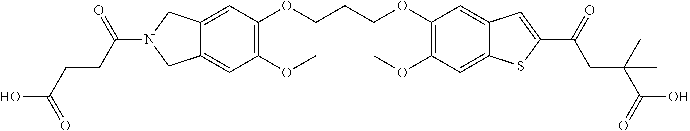 [SMILES: COc1cc2c(cc1OCCCOc1cc3cc(C(=O)CC(C)(C)C(=O)O)sc3cc1OC)CN(C(=O)CCC(=O)O)C2] | 614 |
| 38. | 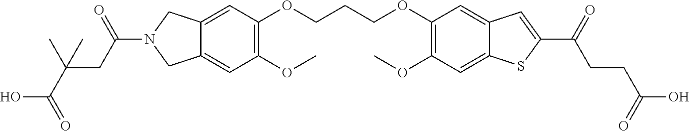 [SMILES: COc1cc2c(cc1OCCCOc1cc3cc(C(=O)CCC(=O)O)sc3cc1OC)CN(C(=O)CC(C)(C)C(=O)O)C2] | 614 |
| 39. | 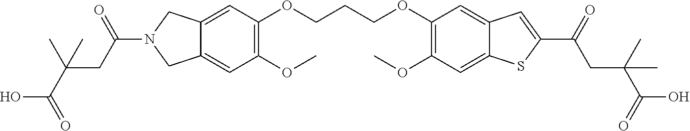 [SMILES: COc1cc2c(cc1OCCCOc1cc3cc(C(=O)CC(C)(C)C(=O)O)sc3cc1OC)CN(C(=O)CC(C)(C)C(=O)O)C2] | 642 |
| 40. | 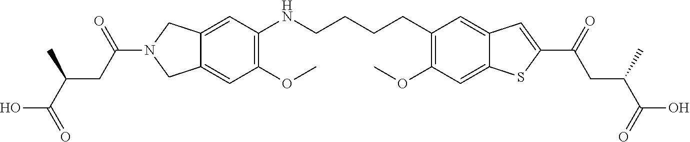 [SMILES: COc1cc2sc(C(=O)C[C@H](C)C(=O)O)cc2cc1CCCCNc1cc2c(cc1OC)CN(C(=O)C[C@H](C)C(=O)O)C2] | 611 |
| 41. | 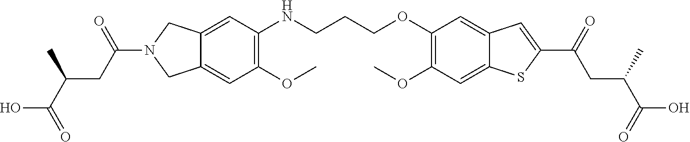 [SMILES: COc1cc2c(cc1NCCCOc1cc3cc(C(=O)C[C@H](C)C(=O)O)sc3cc1OC)CN(C(=O)C[C@H](C)C(=O)O)C2] | 613 |
| 42. | 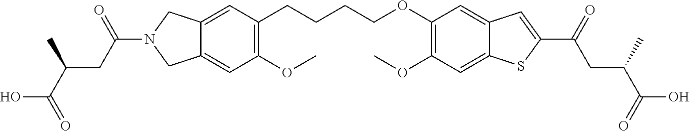 [SMILES: COc1cc2c(cc1CCCCOc1cc3cc(C(=O)C[C@H](C)C(=O)O)sc3cc1OC)CN(C(=O)C[C@H](C)C(=O)O)C2] | 612 |
| 43. | 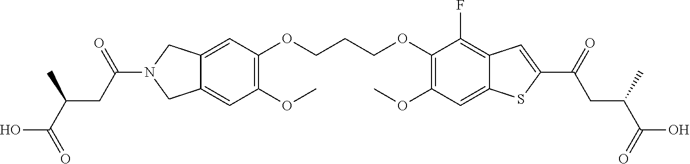 [SMILES: COc1cc2c(cc1OCCCOc1c(OC)cc3sc(C(=O)C[C@H](C)C(=O)O)cc3c1F)CN(C(=O)C[C@H](C)C(=O)O)C2] | 632 |
| 44. | 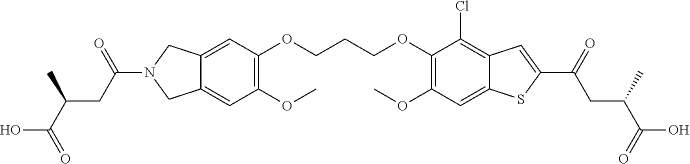 [SMILES: COc1cc2c(cc1OCCCOc1c(OC)cc3sc(C(=O)C[C@H](C)C(=O)O)cc3c1Cl)CN(C(=O)C[C@H](C)C(=O)O)C2] | 648 |
| 45. | 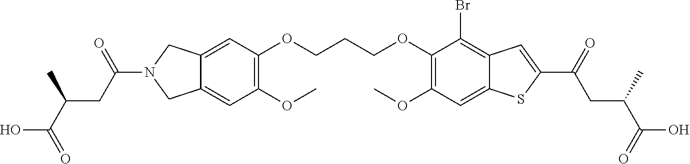 [SMILES: COc1cc2c(cc1OCCCOc1c(OC)cc3sc(C(=O)C[C@H](C)C(=O)O)cc3c1Br)CN(C(=O)C[C@H](C)C(=O)O)C2] | 692 |
| 46. | 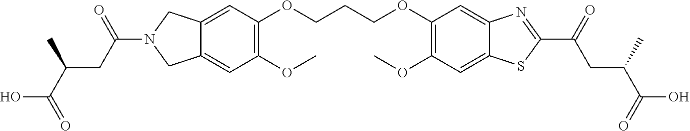 [SMILES: COc1cc2c(cc1OCCCOc1cc3nc(C(=O)C[C@H](C)C(=O)O)sc3cc1OC)CN(C(=O)C[C@H](C)C(=O)O)C2] | 615 |
| 47. | 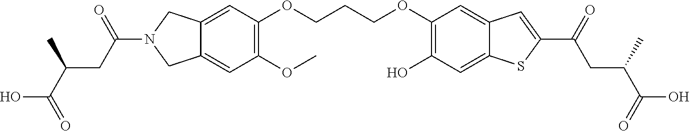 [SMILES: COc1cc2c(cc1OCCCOc1cc3cc(C(=O)C[C@H](C)C(=O)O)sc3cc1O)CN(C(=O)C[C@H](C)C(=O)O)C2] | 600 |
| 48. | 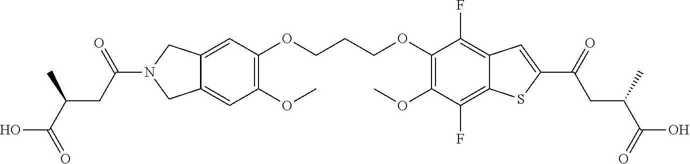 [SMILES: COc1cc2c(cc1OCCCOc1c(OC)c(F)c3sc(C(=O)C[C@H](C)C(=O)O)cc3c1F)CN(C(=O)C[C@H](C)C(=O)O)C2] | 650 |
| 49. | 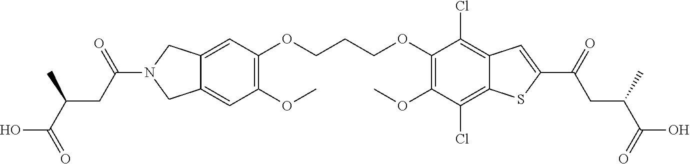 [SMILES: COc1cc2c(cc1OCCCOc1c(OC)c(Cl)c3sc(C(=O)C[C@H](C)C(=O)O)cc3c1Cl)CN(C(=O)C[C@H](C)C(=O)O)C2] | 682 |
| 50. | 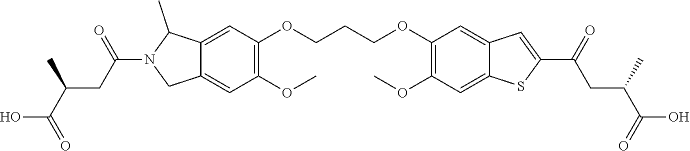 [SMILES: COc1cc2c(cc1OCCCOc1cc3cc(C(=O)C[C@H](C)C(=O)O)sc3cc1OC)C(C)N(C(=O)C[C@H](C)C(=O)O)C2] | 628 |
| 51. | 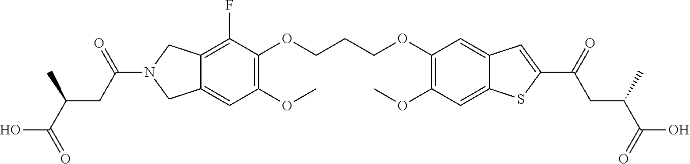 [SMILES: COc1cc2sc(C(=O)C[C@H](C)C(=O)O)cc2cc1OCCCOc1c(OC)cc2c(c1F)CN(C(=O)C[C@H](C)C(=O)O)C2] | 632 |
| 52. | 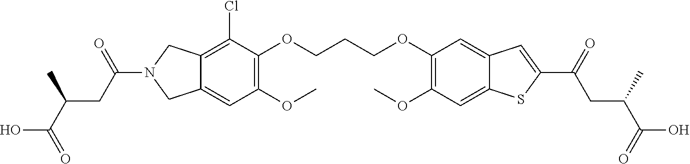 [SMILES: COc1cc2sc(C(=O)C[C@H](C)C(=O)O)cc2cc1OCCCOc1c(OC)cc2c(c1Cl)CN(C(=O)C[C@H](C)C(=O)O)C2] | 648 |
| 53. | 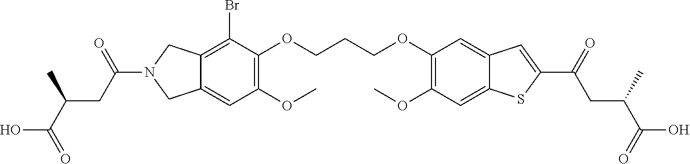 [SMILES: COc1cc2sc(C(=O)C[C@H](C)C(=O)O)cc2cc1OCCCOc1c(OC)cc2c(c1Br)CN(C(=O)C[C@H](C)C(=O)O)C2] | 692 |
| 54. | 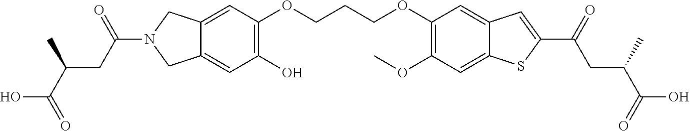 [SMILES: COc1cc2sc(C(=O)C[C@H](C)C(=O)O)cc2cc1OCCCOc1cc2c(cc1O)CN(C(=O)C[C@H](C)C(=O)O)C2] | 600 |
| 55. | 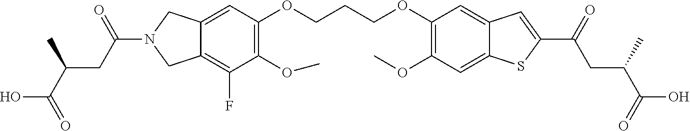 [SMILES: COc1cc2sc(C(=O)C[C@H](C)C(=O)O)cc2cc1OCCCOc1cc2c(c(F)c1OC)CN(C(=O)C[C@H](C)C(=O)O)C2] | 632 |
| 56. | 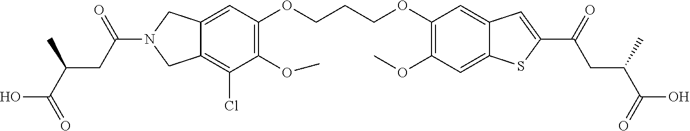 [SMILES: COc1cc2sc(C(=O)C[C@H](C)C(=O)O)cc2cc1OCCCOc1cc2c(c(Cl)c1OC)CN(C(=O)C[C@H](C)C(=O)O)C2] | 648 |
| 57. | 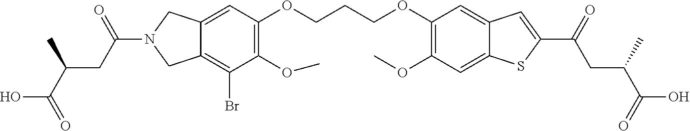 [SMILES: COc1cc2sc(C(=O)C[C@H](C)C(=O)O)cc2cc1OCCCOc1cc2c(c(Br)c1OC)CN(C(=O)C[C@H](C)C(=O)O)C2] | 692 |
| 58. | 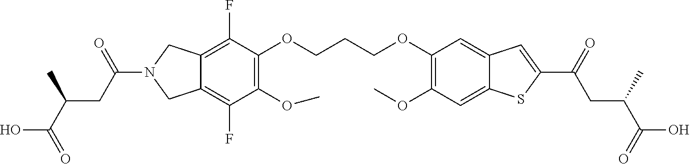 [SMILES: COc1cc2sc(C(=O)C[C@H](C)C(=O)O)cc2cc1OCCCOc1c(F)c2c(c(F)c1OC)CN(C(=O)C[C@H](C)C(=O)O)C2] | 650 |
| 59. | 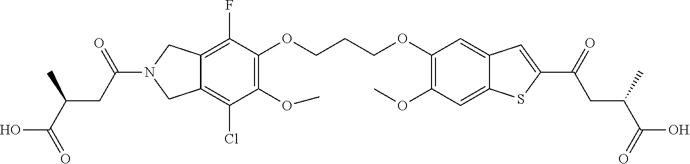 [SMILES: COc1cc2sc(C(=O)C[C@H](C)C(=O)O)cc2cc1OCCCOc1c(F)c2c(c(Cl)c1OC)CN(C(=O)C[C@H](C)C(=O)O)C2] | 666 |
| 60. | 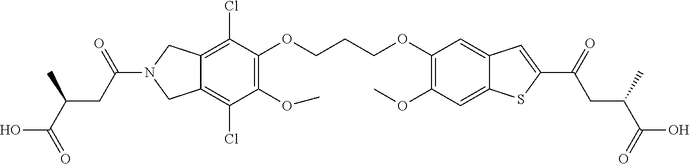 [SMILES: COc1cc2sc(C(=O)C[C@H](C)C(=O)O)cc2cc1OCCCOc1c(Cl)c2c(c(Cl)c1OC)CN(C(=O)C[C@H](C)C(=O)O)C2] | 682 |
| 61. | 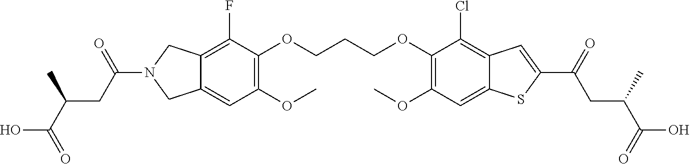 [SMILES: COc1cc2c(c(F)c1OCCCOc1c(OC)cc3sc(C(=O)C[C@H](C)C(=O)O)cc3c1Cl)CN(C(=O)C[C@H](C)C(=O)O)C2] | 666 |
| 62. | 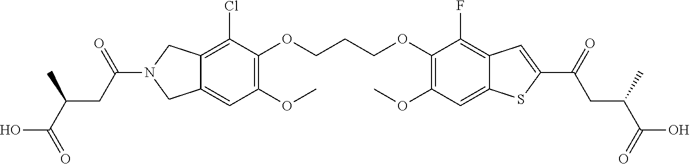 [SMILES: COc1cc2c(c(Cl)c1OCCCOc1c(OC)cc3sc(C(=O)C[C@H](C)C(=O)O)cc3c1F)CN(C(=O)C[C@H](C)C(=O)O)C2] | 666 |
| 63. | 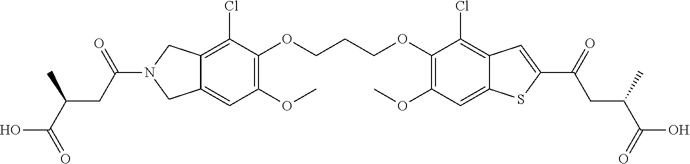 [SMILES: COc1cc2c(c(Cl)c1OCCCOc1c(OC)cc3sc(C(=O)C[C@H](C)C(=O)O)cc3c1Cl)CN(C(=O)C[C@H](C)C(=O)O)C2] | 682 |
| 64. | 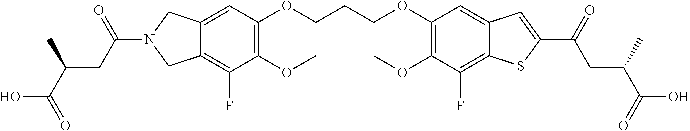 [SMILES: COc1c(OCCCOc2cc3cc(C(=O)C[C@H](C)C(=O)O)sc3c(F)c2OC)cc2c(c1F)CN(C(=O)C[C@H](C)C(=O)O)C2] | 650 |
| 65. | 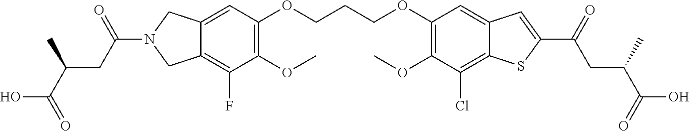 [SMILES: COc1c(OCCCOc2cc3cc(C(=O)C[C@H](C)C(=O)O)sc3c(Cl)c2OC)cc2c(c1F)CN(C(=O)C[C@H](C)C(=O)O)C2] | 666 |
| 66. | 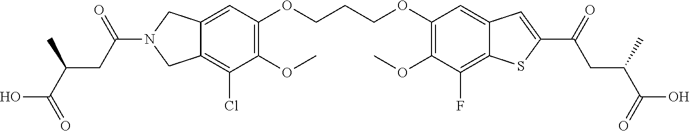 [SMILES: COc1c(OCCCOc2cc3cc(C(=O)C[C@H](C)C(=O)O)sc3c(F)c2OC)cc2c(c1Cl)CN(C(=O)C[C@H](C)C(=O)O)C2] | 666 |
| 67. | 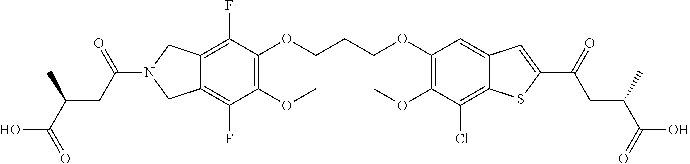 [SMILES: COc1c(F)c2c(c(F)c1OCCCOc1cc3cc(C(=O)C[C@H](C)C(=O)O)sc3c(Cl)c1OC)CN(C(=O)C[C@H](C)C(=O)O)C2] | 684 |
| 68. | 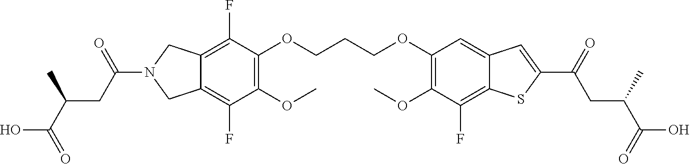 [SMILES: COc1c(F)c2c(c(F)c1OCCCOc1cc3cc(C(=O)C[C@H](C)C(=O)O)sc3c(F)c1OC)CN(C(=O)C[C@H](C)C(=O)O)C2] | 668 |
| 69. | 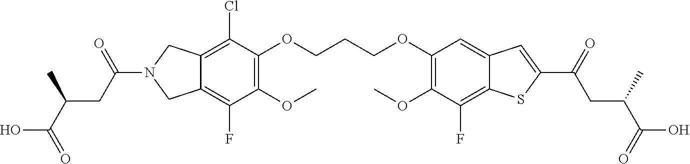 [SMILES: COc1c(F)c2c(c(Cl)c1OCCCOc1cc3cc(C(=O)C[C@H](C)C(=O)O)sc3c(F)c1OC)CN(C(=O)C[C@H](C)C(=O)O)C2] | 684 |
| 70. | 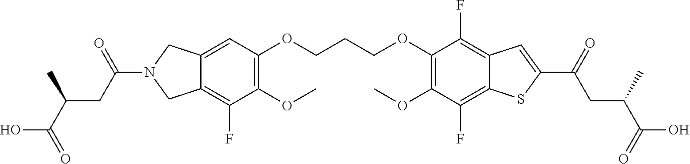 [SMILES: COc1c(OCCCOc2c(OC)c(F)c3sc(C(=O)C[C@H](C)C(=O)O)cc3c2F)cc2c(c1F)CN(C(=O)C[C@H](C)C(=O)O)C2] | 668 |
| 71. | 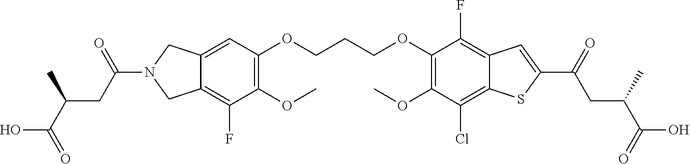 [SMILES: COc1c(OCCCOc2c(OC)c(Cl)c3sc(C(=O)C[C@H](C)C(=O)O)cc3c2F)cc2c(c1F)CN(C(=O)C[C@H](C)C(=O)O)C2] | 684 |
| 72. | 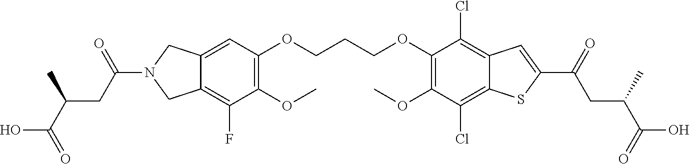 [SMILES: COc1c(OCCCOc2c(OC)c(Cl)c3sc(C(=O)C[C@H](C)C(=O)O)cc3c2Cl)cc2c(c1F)CN(C(=O)C[C@H](C)C(=O)O)C2] | 700 |
| 73. | 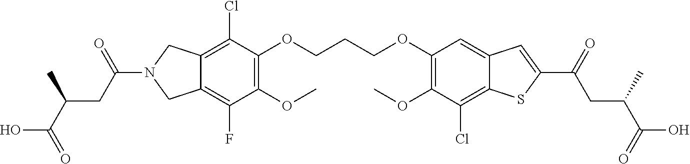 [SMILES: COc1c(F)c2c(c(Cl)c1OCCCOc1cc3cc(C(=O)C[C@H](C)C(=O)O)sc3c(Cl)c1OC)CN(C(=O)C[C@H](C)C(=O)O)C2] | 700 |
| 74. | 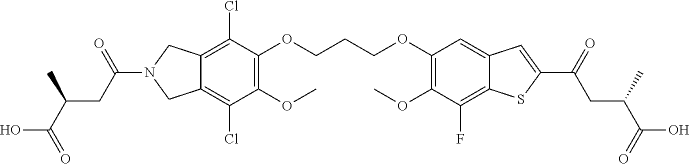 [SMILES: COc1c(Cl)c2c(c(Cl)c1OCCCOc1cc3cc(C(=O)C[C@H](C)C(=O)O)sc3c(F)c1OC)CN(C(=O)C[C@H](C)C(=O)O)C2] | 700 |
| 75. | 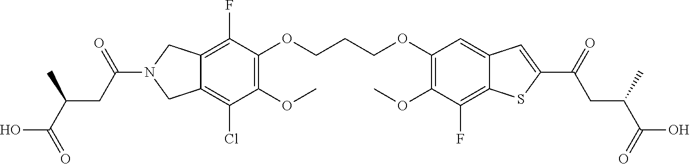 [SMILES: COc1c(Cl)c2c(c(F)c1OCCCOc1cc3cc(C(=O)C[C@H](C)C(=O)O)sc3c(F)c1OC)CN(C(=O)C[C@H](C)C(=O)O)C2] | 684 |
| 76. | 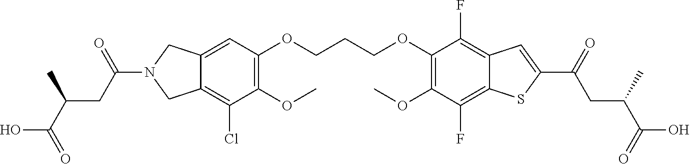 [SMILES: COc1c(OCCCOc2c(OC)c(F)c3sc(C(=O)C[C@H](C)C(=O)O)cc3c2F)cc2c(c1Cl)CN(C(=O)C[C@H](C)C(=O)O)C2] | 684 |
| 77. | 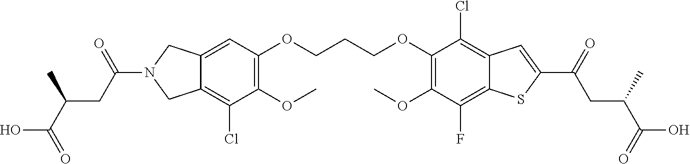 [SMILES: COc1c(OCCCOc2c(OC)c(F)c3sc(C(=O)C[C@H](C)C(=O)O)cc3c2Cl)cc2c(c1Cl)CN(C(=O)C[C@H](C)C(=O)O)C2] | 700 |
| 78. | 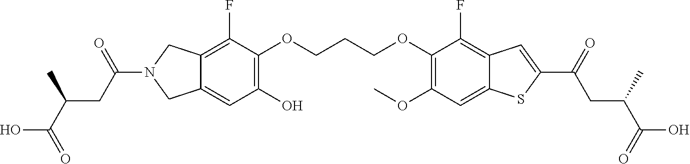 [SMILES: COc1cc2sc(C(=O)C[C@H](C)C(=O)O)cc2c(F)c1OCCCOc1c(O)cc2c(c1F)CN(C(=O)C[C@H](C)C(=O)O)C2] | 636 |
| 79. | 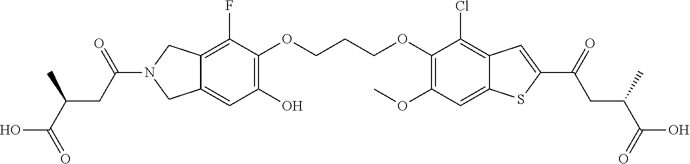 [SMILES: COc1cc2sc(C(=O)C[C@H](C)C(=O)O)cc2c(Cl)c1OCCCOc1c(O)cc2c(c1F)CN(C(=O)C[C@H](C)C(=O)O)C2] | 652 |
| 80. | 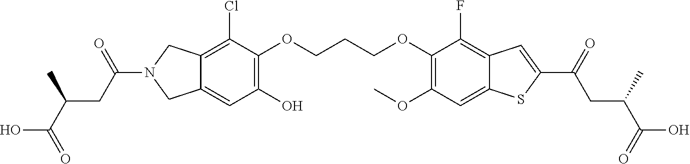 [SMILES: COc1cc2sc(C(=O)C[C@H](C)C(=O)O)cc2c(F)c1OCCCOc1c(O)cc2c(c1Cl)CN(C(=O)C[C@H](C)C(=O)O)C2] | 652 |
| 81. | 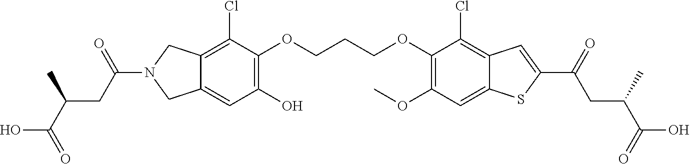 [SMILES: COc1cc2sc(C(=O)C[C@H](C)C(=O)O)cc2c(Cl)c1OCCCOc1c(O)cc2c(c1Cl)CN(C(=O)C[C@H](C)C(=O)O)C2] | 668 |
| 82. | 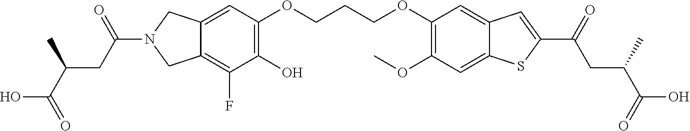 [SMILES: COc1cc2sc(C(=O)C[C@H](C)C(=O)O)cc2cc1OCCCOc1cc2c(c(F)c1O)CN(C(=O)C[C@H](C)C(=O)O)C2] | 618 |
| 83. | 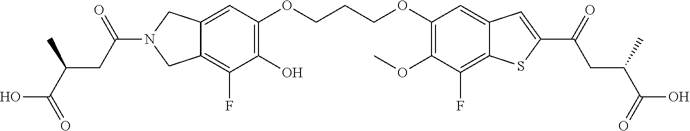 [SMILES: COc1c(OCCCOc2cc3c(c(F)c2O)CN(C(=O)C[C@H](C)C(=O)O)C3)cc2cc(C(=O)C[C@H](C)C(=O)O)sc2c1F] | 636 |
| 84. | 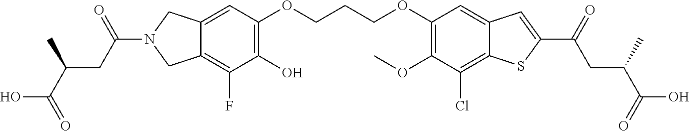 [SMILES: COc1c(OCCCOc2cc3c(c(F)c2O)CN(C(=O)C[C@H](C)C(=O)O)C3)cc2cc(C(=O)C[C@H](C)C(=O)O)sc2c1Cl] | 652 |
| 85. | 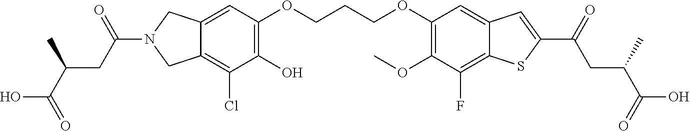 [SMILES: COc1c(OCCCOc2cc3c(c(Cl)c2O)CN(C(=O)C[C@H](C)C(=O)O)C3)cc2cc(C(=O)C[C@H](C)C(=O)O)sc2c1F] | 652 |
| 86. | 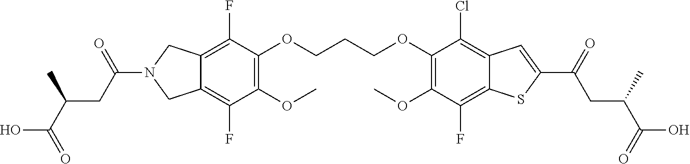 [SMILES: COc1c(F)c2c(c(F)c1OCCCOc1c(OC)c(F)c3sc(C(=O)C[C@H](C)C(=O)O)cc3c1Cl)CN(C(=O)C[C@H](C)C(=O)O)C2] | 702 |
| 87. | 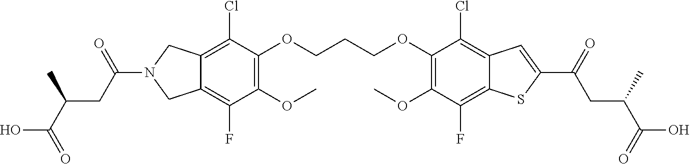 [SMILES: COc1c(F)c2c(c(Cl)c1OCCCOc1c(OC)c(F)c3sc(C(=O)C[C@H](C)C(=O)O)cc3c1Cl)CN(C(=O)C[C@H](C)C(=O)O)C2] | 718 |
| 88. | 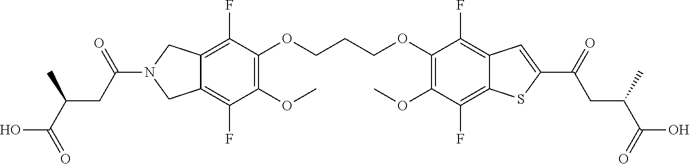 [SMILES: COc1c(F)c2c(c(F)c1OCCCOc1c(OC)c(F)c3sc(C(=O)C[C@H](C)C(=O)O)cc3c1F)CN(C(=O)C[C@H](C)C(=O)O)C2] | 686 |
| 89. | 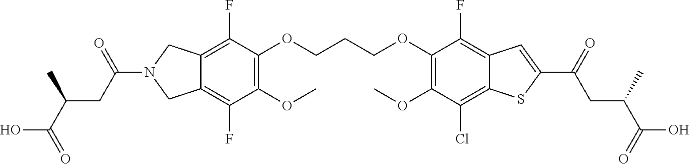 [SMILES: COc1c(F)c2c(c(F)c1OCCCOc1c(OC)c(Cl)c3sc(C(=O)C[C@H](C)C(=O)O)cc3c1F)CN(C(=O)C[C@H](C)C(=O)O)C2] | 702 |
| 90. | 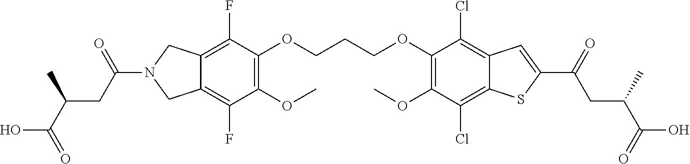 [SMILES: COc1c(F)c2c(c(F)c1OCCCOc1c(OC)c(Cl)c3sc(C(=O)C[C@H](C)C(=O)O)cc3c1Cl)CN(C(=O)C[C@H](C)C(=O)O)C2] | 718 |
| 91. | 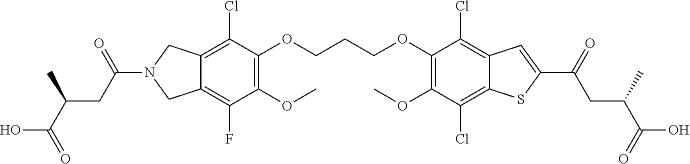 [SMILES: COc1c(F)c2c(c(Cl)c1OCCCOc1c(OC)c(Cl)c3sc(C(=O)C[C@H](C)C(=O)O)cc3c1Cl)CN(C(=O)C[C@H](C)C(=O)O)C2] | 734 |
| 92. | 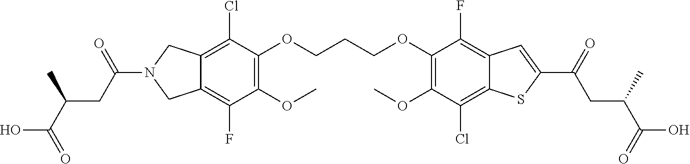 [SMILES: COc1c(F)c2c(c(Cl)c1OCCCOc1c(OC)c(Cl)c3sc(C(=O)C[C@H](C)C(=O)O)cc3c1F)CN(C(=O)C[C@H](C)C(=O)O)C2] | 718 |
| 93. | 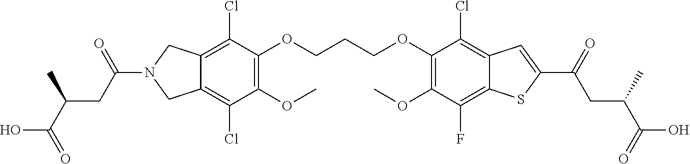 [SMILES: COc1c(Cl)c2c(c(Cl)c1OCCCOc1c(OC)c(F)c3sc(C(=O)C[C@H](C)C(=O)O)cc3c1Cl)CN(C(=O)C[C@H](C)C(=O)O)C2] | 734 |
| 94. | 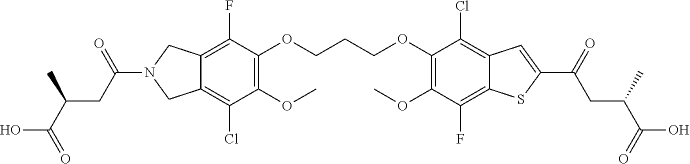 [SMILES: COc1c(Cl)c2c(c(F)c1OCCCOc1c(OC)c(F)c3sc(C(=O)C[C@H](C)C(=O)O)cc3c1Cl)CN(C(=O)C[C@H](C)C(=O)O)C2] | 718 |
| 95. | 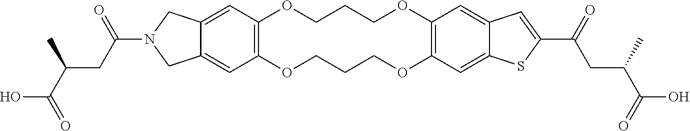 [SMILES: C[C@@H](CC(=O)c1cc2cc3c(cc2s1)OCCCOc1cc2c(cc1OCCCO3)CN(C(=O)C[C@H](C)C(=O)O)C2)C(=O)O] | 626 |
| 96. | 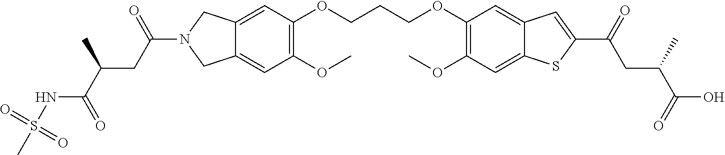 [SMILES: COc1cc2c(cc1OCCCOc1cc3cc(C(=O)C[C@H](C)C(=O)O)sc3cc1OC)CN(C(=O)C[C@H](C)C(=O)NS(C)(=O)=O)C2] | 691 |
| 97. | 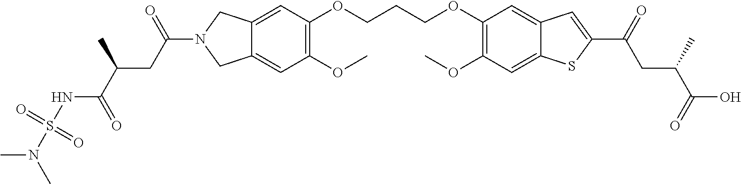 [SMILES: COc1cc2c(cc1OCCCOc1cc3cc(C(=O)C[C@H](C)C(=O)O)sc3cc1OC)CN(C(=O)C[C@H](C)C(=O)NS(=O)(=O)N(C)C)C2] | 720 |
| 98. | 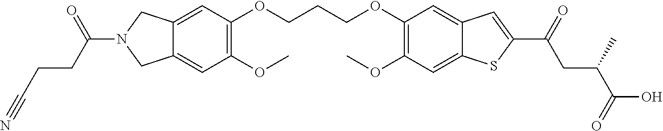 [SMILES: COc1cc2c(cc1OCCCOc1cc3cc(C(=O)C[C@H](C)C(=O)O)sc3cc1OC)CN(C(=O)CCC#N)C2] | 581 |
| 99. | 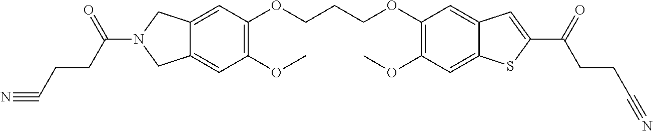 [SMILES: COc1cc2c(cc1OCCCOc1cc3cc(C(=O)CCC#N)sc3cc1OC)CN(C(=O)CCC#N)C2] | 548 |
| 100. | 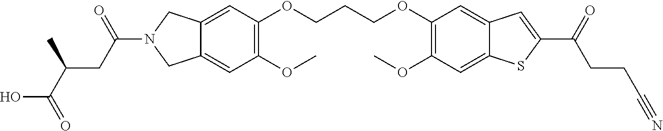 [SMILES: COc1cc2c(cc1OCCCOc1cc3cc(C(=O)CCC#N)sc3cc1OC)CN(C(=O)C[C@H](C)C(=O)O)C2] | 581 |
| 101. | 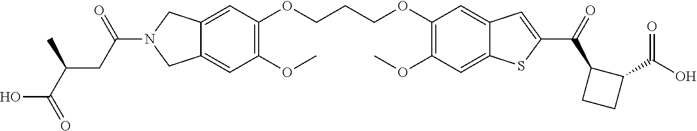 [SMILES: COc1cc2c(cc1OCCCOc1cc3cc(C(=O)[C@@H]4CC[C@H]4C(=O)O)sc3cc1OC)CN(C(=O)C[C@H](C)C(=O)O)C2] | 626 |
| 102. | 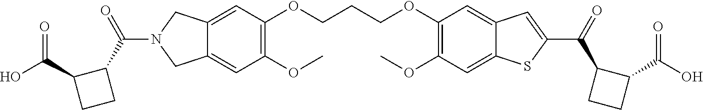 [SMILES: COc1cc2c(cc1OCCCOc1cc3cc(C(=O)[C@@H]4CC[C@H]4C(=O)O)sc3cc1OC)CN(C(=O)[C@@H]1CC[C@H]1C(=O)O)C2] | 638 |
| 103. | 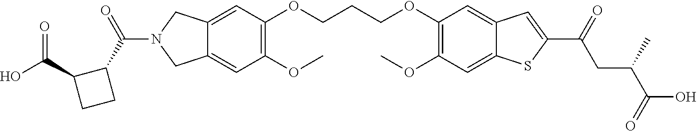 [SMILES: COc1cc2c(cc1OCCCOc1cc3cc(C(=O)C[C@H](C)C(=O)O)sc3cc1OC)CN(C(=O)[C@@H]1CC[C@H]1C(=O)O)C2] | 626 |
| 104. | 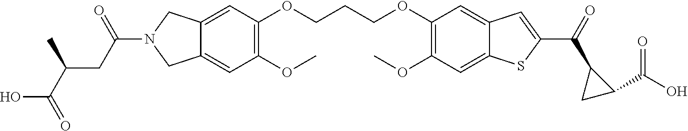 [SMILES: COc1cc2c(cc1OCCCOc1cc3cc(C(=O)[C@@H]4C[C@H]4C(=O)O)sc3cc1OC)CN(C(=O)C[C@H](C)C(=O)O)C2] | 612 |
| 105. | 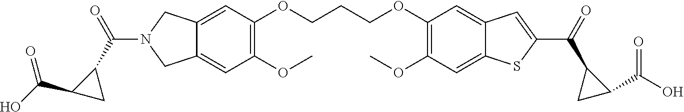 [SMILES: COc1cc2c(cc1OCCCOc1cc3cc(C(=O)[C@@H]4C[C@H]4C(=O)O)sc3cc1OC)CN(C(=O)[C@@H]1C[C@H]1C(=O)O)C2] | 610 |
| 106. | 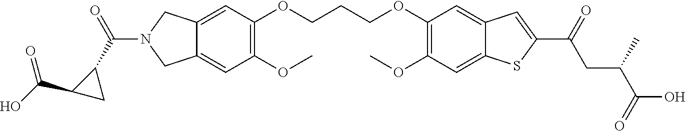 [SMILES: COc1cc2c(cc1OCCCOc1cc3cc(C(=O)C[C@H](C)C(=O)O)sc3cc1OC)CN(C(=O)[C@@H]1C[C@H]1C(=O)O)C2] | 612 |
| 107. | 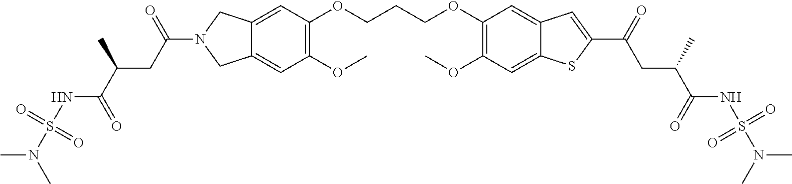 | 826 |
| 108. | 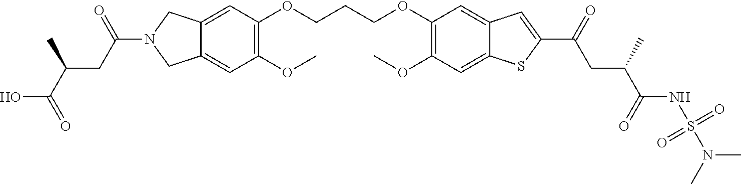 [SMILES: COc1cc2c(cc1OCCCOc1cc3cc(C(=O)C[C@H](C)C(=O)NS(=O)(=O)N(C)C)sc3cc1OC)CN(C(=O)C[C@H](C)C(=O)O)C2] | 720 |
| 109. | 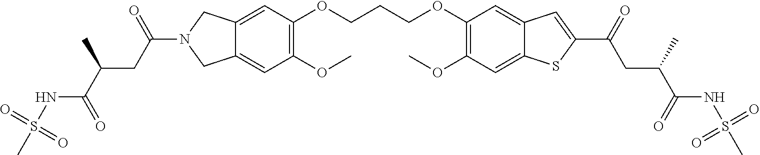 [SMILES: COc1cc2c(cc1OCCCOc1cc3cc(C(=O)C[C@H](C)C(=O)NS(C)(=O)=O)sc3cc1OC)CN(C(=O)C[C@H](C)C(=O)NS(C)(=O)=O)C2] | 768 |
| 110. | 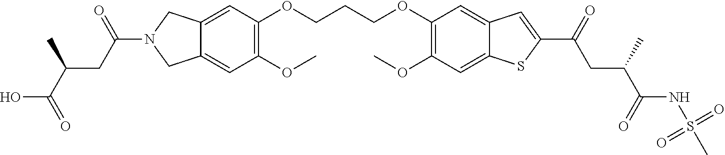 [SMILES: COc1cc2c(cc1OCCCOc1cc3cc(C(=O)C[C@H](C)C(=O)NS(C)(=O)=O)sc3cc1OC)CN(C(=O)C[C@H](C)C(=O)O)C2] | 691 |
| 111. | 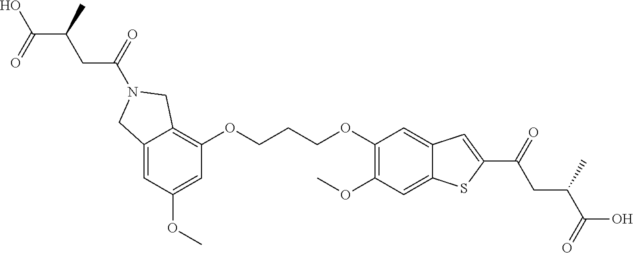 [SMILES: COc1cc2c(c(OCCCOc3cc4cc(C(=O)C[C@H](C)C(=O)O)sc4cc3OC)c1)CN(C(=O)C[C@H](C)C(=O)O)C2] | 614 |
| 112. | 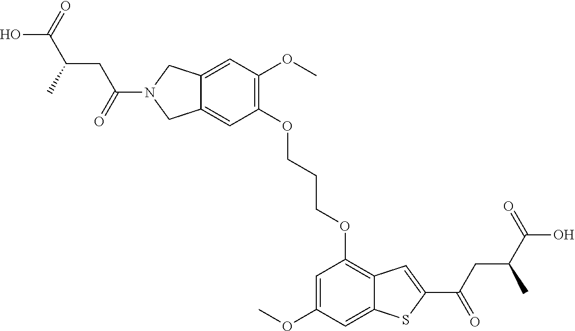 [SMILES: COc1cc(OCCCOc2cc3c(cc2OC)CN(C(=O)C[C@H](C)C(=O)O)C3)c2cc(C(=O)C[C@H](C)C(=O)O)sc2c1] | 614 |
| 113. | 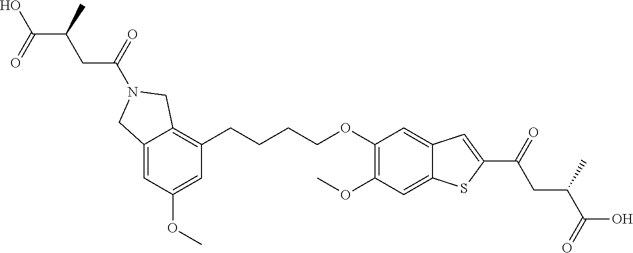 [SMILES: COc1cc(CCCCOc2cc3cc(C(=O)C[C@H](C)C(=O)O)sc3cc2OC)c2c(c1)CN(C(=O)C[C@H](C)C(=O)O)C2] | 612 |
| 114. | 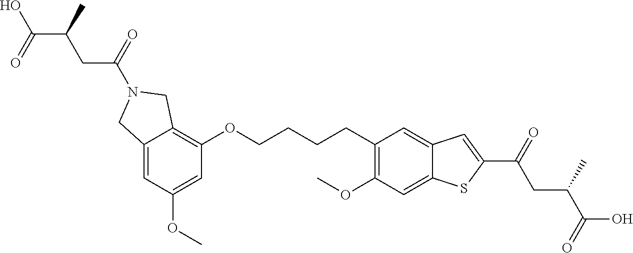 [SMILES: COc1cc2c(c(OCCCCc3cc4cc(C(=O)C[C@H](C)C(=O)O)sc4cc3OC)c1)CN(C(=O)C[C@H](C)C(=O)O)C2] | 612 |
| 115. | 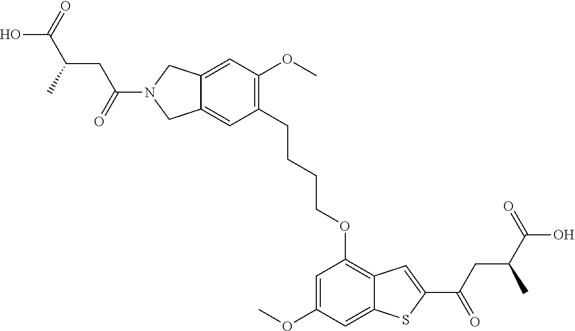 [SMILES: COc1cc(OCCCCc2cc3c(cc2OC)CN(C(=O)C[C@H](C)C(=O)O)C3)c2cc(C(=O)C[C@H](C)C(=O)O)sc2c1] | 612 |
| 116. | 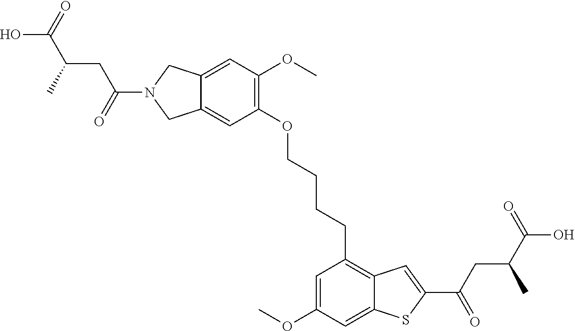 [SMILES: COc1cc(CCCCOc2cc3c(cc2OC)CN(C(=O)C[C@H](C)C(=O)O)C3)c2cc(C(=O)C[C@H](C)C(=O)O)sc2c1] | 612 |
| 117. | 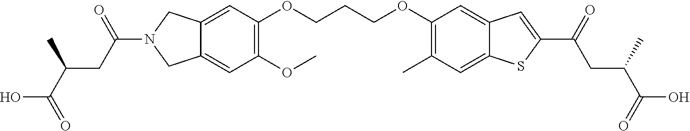 [SMILES: COc1cc2c(cc1OCCCOc1cc3cc(C(=O)C[C@H](C)C(=O)O)sc3cc1C)CN(C(=O)C[C@H](C)C(=O)O)C2] | 598 |
| 118. | 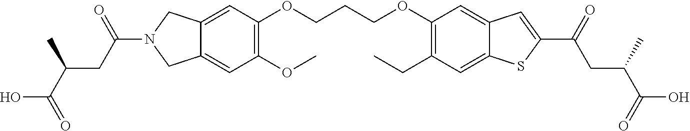 [SMILES: CCc1cc2sc(C(=O)C[C@H](C)C(=O)O)cc2cc1OCCCOc1cc2c(cc1OC)CN(C(=O)C[C@H](C)C(=O)O)C2] | 612 |
| 119. | 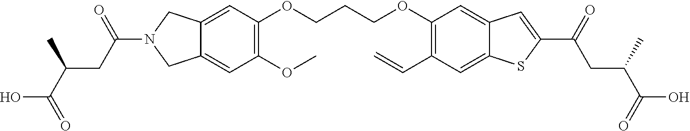 [SMILES: C=Cc1cc2sc(C(=O)C[C@H](C)C(=O)O)cc2cc1OCCCOc1cc2c(cc1OC)CN(C(=O)C[C@H](C)C(=O)O)C2] | 610 |
| 120. | 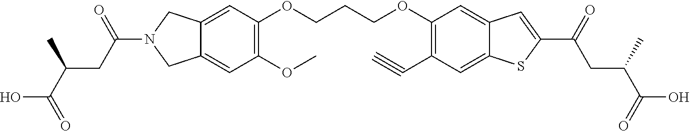 [SMILES: C#Cc1cc2sc(C(=O)C[C@H](C)C(=O)O)cc2cc1OCCCOc1cc2c(cc1OC)CN(C(=O)C[C@H](C)C(=O)O)C2] | 608 |
| 121. | 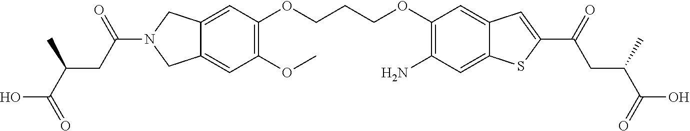 [SMILES: COc1cc2c(cc1OCCCOc1cc3cc(C(=O)C[C@H](C)C(=O)O)sc3cc1N)CN(C(=O)C[C@H](C)C(=O)O)C2] | 599 |
| 122. | 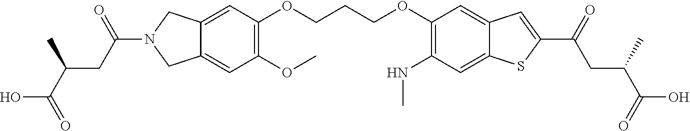 [SMILES: CNc1cc2sc(C(=O)C[C@H](C)C(=O)O)cc2cc1OCCCOc1cc2c(cc1OC)CN(C(=O)C[C@H](C)C(=O)O)C2] | 613 |
| 123. | 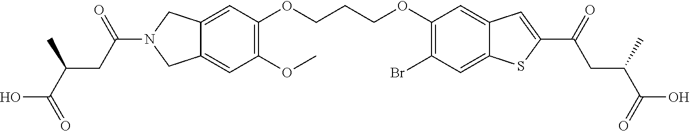 [SMILES: COc1cc2c(cc1OCCCOc1cc3cc(C(=O)C[C@H](C)C(=O)O)sc3cc1Br)CN(C(=O)C[C@H](C)C(=O)O)C2] | 662 |
| 124. | 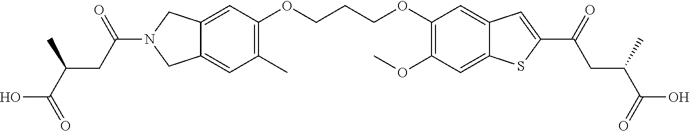 [SMILES: COc1cc2sc(C(=O)C[C@H](C)C(=O)O)cc2cc1OCCCOc1cc2c(cc1C)CN(C(=O)C[C@H](C)C(=O)O)C2] | 598 |
| 125. | 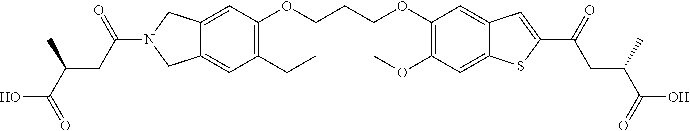 [SMILES: CCc1cc2c(cc1OCCCOc1cc3cc(C(=O)C[C@H](C)C(=O)O)sc3cc1OC)CN(C(=O)C[C@H](C)C(=O)O)C2] | 612 |
| 126. | 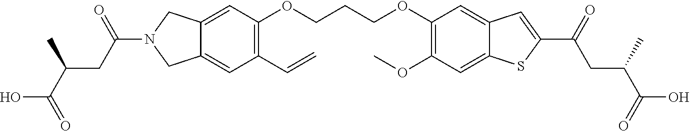 [SMILES: C=Cc1cc2c(cc1OCCCOc1cc3cc(C(=O)C[C@H](C)C(=O)O)sc3cc1OC)CN(C(=O)C[C@H](C)C(=O)O)C2] | 610 |
| 127. | 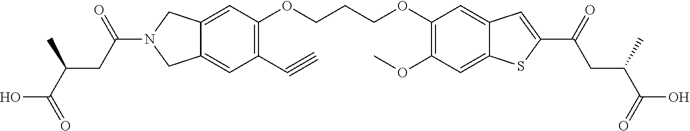 [SMILES: C#Cc1cc2c(cc1OCCCOc1cc3cc(C(=O)C[C@H](C)C(=O)O)sc3cc1OC)CN(C(=O)C[C@H](C)C(=O)O)C2] | 608 |
| 128. | 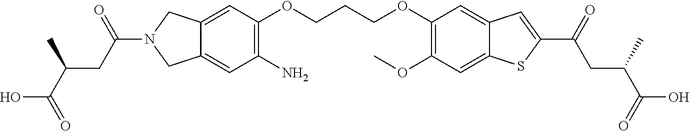 [SMILES: COc1cc2sc(C(=O)C[C@H](C)C(=O)O)cc2cc1OCCCOc1cc2c(cc1N)CN(C(=O)C[C@H](C)C(=O)O)C2] | 599 |
| 129. | 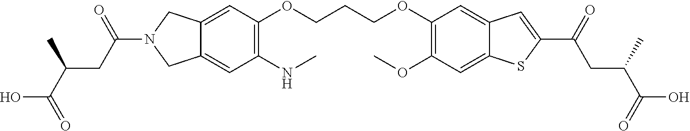 [SMILES: CNc1cc2c(cc1OCCCOc1cc3cc(C(=O)C[C@H](C)C(=O)O)sc3cc1OC)CN(C(=O)C[C@H](C)C(=O)O)C2] | 613 |
| 130. | 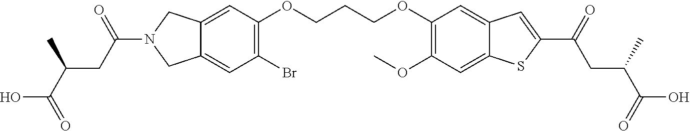 [SMILES: COc1cc2sc(C(=O)C[C@H](C)C(=O)O)cc2cc1OCCCOc1cc2c(cc1Br)CN(C(=O)C[C@H](C)C(=O)O)C2] | 662 |
| 131. | 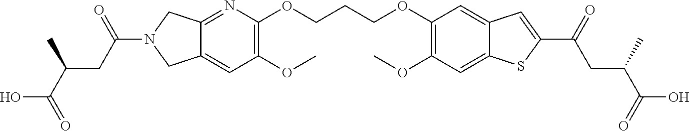 [SMILES: COc1cc2sc(C(=O)C[C@H](C)C(=O)O)cc2cc1OCCCOc1nc2c(cc1OC)CN(C(=O)C[C@H](C)C(=O)O)C2] | 615 |
| 132. | 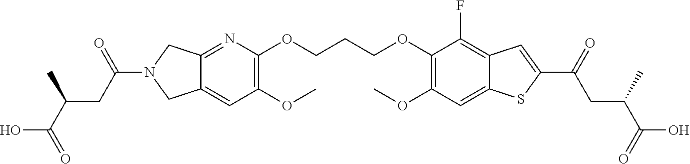 [SMILES: COc1cc2c(nc1OCCCOc1c(OC)cc3sc(C(=O)C[C@H](C)C(=O)O)cc3c1F)CN(C(=O)C[C@H](C)C(=O)O)C2] | 633 |
| 133. | 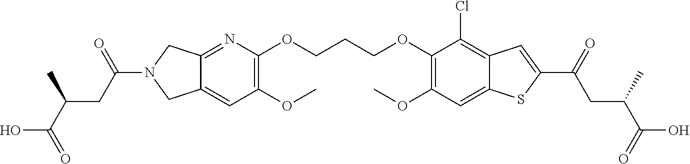 [SMILES: COc1cc2c(nc1OCCCOc1c(OC)cc3sc(C(=O)C[C@H](C)C(=O)O)cc3c1Cl)CN(C(=O)C[C@H](C)C(=O)O)C2] | 649 |
| 134. | 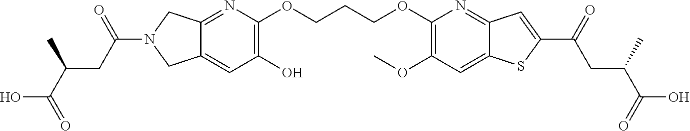 [SMILES: COc1cc2sc(C(=O)C[C@H](C)C(=O)O)cc2nc1OCCCOc1nc2c(cc1O)CN(C(=O)C[C@H](C)C(=O)O)C2] | 602 |
| 135. | 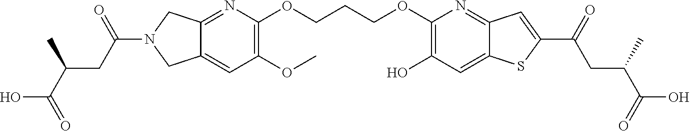 [SMILES: COc1cc2c(nc1OCCCOc1nc3cc(C(=O)C[C@H](C)C(=O)O)sc3cc1O)CN(C(=O)C[C@H](C)C(=O)O)C2] | 602 |
| 136. | 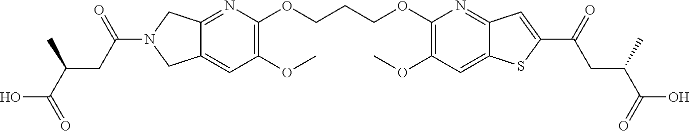 [SMILES: COc1cc2c(nc1OCCCOc1nc3cc(C(=O)C[C@H](C)C(=O)O)sc3cc1OC)CN(C(=O)C[C@H](C)C(=O)O)C2] | 616 |
| 137. | 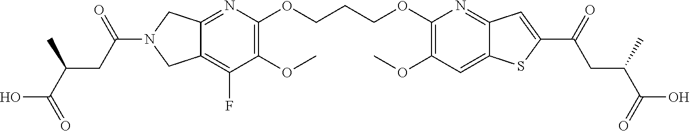 [SMILES: COc1cc2sc(C(=O)C[C@H](C)C(=O)O)cc2nc1OCCCOc1nc2c(c(F)c1OC)CN(C(=O)C[C@H](C)C(=O)O)C2] | 634 |
| 138. | 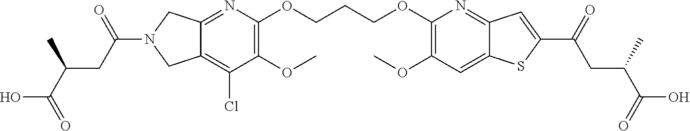 [SMILES: COc1cc2sc(C(=O)C[C@H](C)C(=O)O)cc2nc1OCCCOc1nc2c(c(Cl)c1OC)CN(C(=O)C[C@H](C)C(=O)O)C2] | 650 |
| 139. | 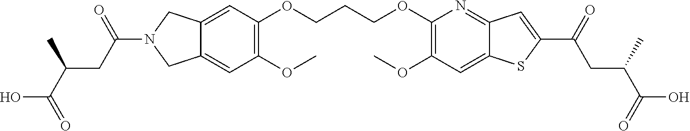 [SMILES: COc1cc2c(cc1OCCCOc1nc3cc(C(=O)C[C@H](C)C(=O)O)sc3cc1OC)CN(C(=O)C[C@H](C)C(=O)O)C2] | 615 |
| 140. | 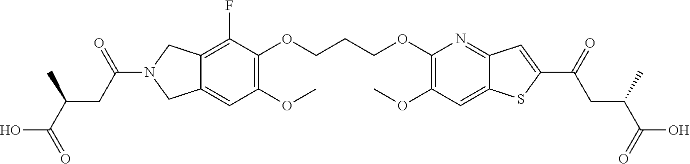 [SMILES: COc1cc2sc(C(=O)C[C@H](C)C(=O)O)cc2nc1OCCCOc1c(OC)cc2c(c1F)CN(C(=O)C[C@H](C)C(=O)O)C2] | 633 |
| 141. | 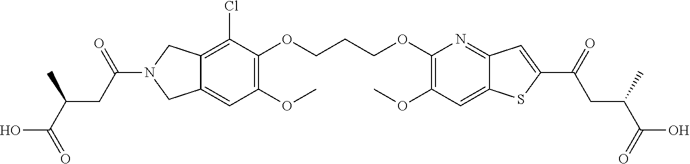 [SMILES: COc1cc2sc(C(=O)C[C@H](C)C(=O)O)cc2nc1OCCCOc1c(OC)cc2c(c1Cl)CN(C(=O)C[C@H](C)C(=O)O)C2] | 649 |
| 142. | 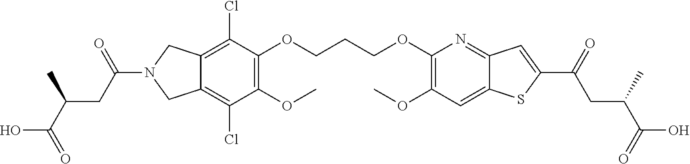 [SMILES: COc1cc2sc(C(=O)C[C@H](C)C(=O)O)cc2nc1OCCCOc1c(Cl)c2c(c(Cl)c1OC)CN(C(=O)C[C@H](C)C(=O)O)C2] | 683 |
| 143. | 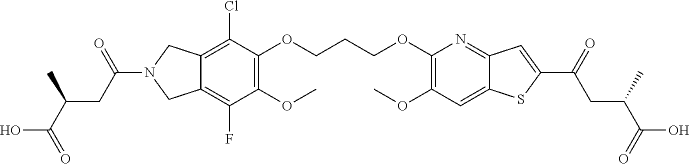 [SMILES: COc1cc2sc(C(=O)C[C@H](C)C(=O)O)cc2nc1OCCCOc1c(Cl)c2c(c(F)c1OC)CN(C(=O)C[C@H](C)C(=O)O)C2] | 667 |
| 144. | 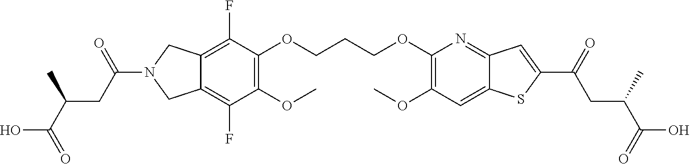 [SMILES: COc1cc2sc(C(=O)C[C@H](C)C(=O)O)cc2nc1OCCCOc1c(F)c2c(c(F)c1OC)CN(C(=O)C[C@H](C)C(=O)O)C2] | 651 |
| 145. | 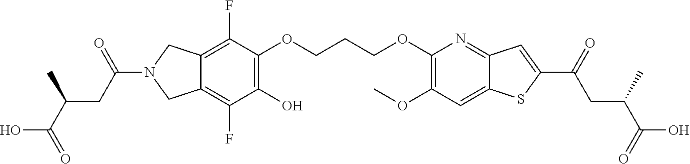 [SMILES: COc1cc2sc(C(=O)C[C@H](C)C(=O)O)cc2nc1OCCCOc1c(O)c(F)c2c(c1F)CN(C(=O)C[C@H](C)C(=O)O)C2] | 637 |
| 146. | 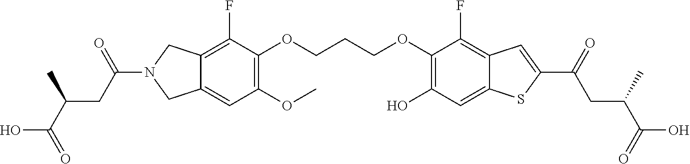 [SMILES: COc1cc2c(c(F)c1OCCCOc1c(O)cc3sc(C(=O)C[C@H](C)C(=O)O)cc3c1F)CN(C(=O)C[C@H](C)C(=O)O)C2] | 636 |
| 147. | 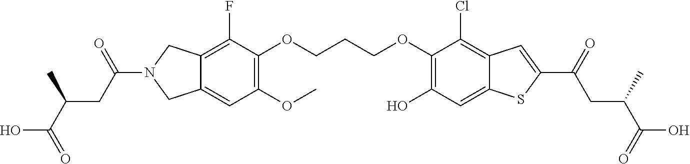 [SMILES: COc1cc2c(c(F)c1OCCCOc1c(O)cc3sc(C(=O)C[C@H](C)C(=O)O)cc3c1Cl)CN(C(=O)C[C@H](C)C(=O)O)C2] | 652 |
| 148. | 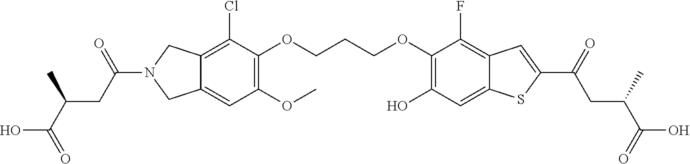 [SMILES: COc1cc2c(c(Cl)c1OCCCOc1c(O)cc3sc(C(=O)C[C@H](C)C(=O)O)cc3c1F)CN(C(=O)C[C@H](C)C(=O)O)C2] | 652 |
| 149. | 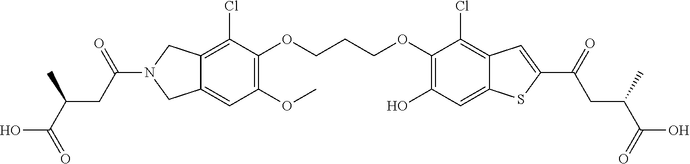 [SMILES: COc1cc2c(c(Cl)c1OCCCOc1c(O)cc3sc(C(=O)C[C@H](C)C(=O)O)cc3c1Cl)CN(C(=O)C[C@H](C)C(=O)O)C2] | 668 |
| 150. | 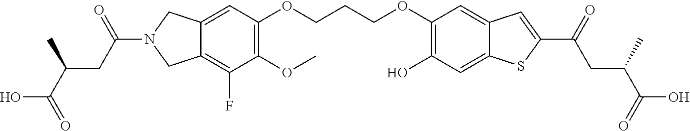 [SMILES: COc1c(OCCCOc2cc3cc(C(=O)C[C@H](C)C(=O)O)sc3cc2O)cc2c(c1F)CN(C(=O)C[C@H](C)C(=O)O)C2] | 618 |
| 151. | 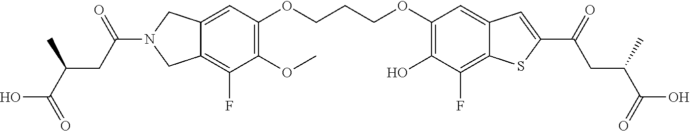 [SMILES: COc1c(OCCCOc2cc3cc(C(=O)C[C@H](C)C(=O)O)sc3c(F)c2O)cc2c(c1F)CN(C(=O)C[C@H](C)C(=O)O)C2] | 636 |
| 152. | 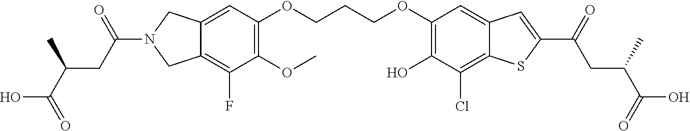 [SMILES: COc1c(OCCCOc2cc3cc(C(=O)C[C@H](C)C(=O)O)sc3c(Cl)c2O)cc2c(c1F)CN(C(=O)C[C@H](C)C(=O)O)C2] | 652 |
| 153. | 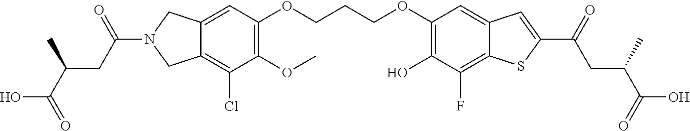 [SMILES: COc1c(OCCCOc2cc3cc(C(=O)C[C@H](C)C(=O)O)sc3c(F)c2O)cc2c(c1Cl)CN(C(=O)C[C@H](C)C(=O)O)C2] | 652 |
| 154. | 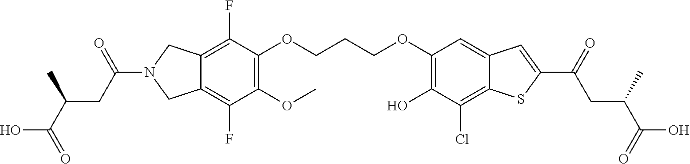 [SMILES: COc1c(F)c2c(c(F)c1OCCCOc1cc3cc(C(=O)C[C@H](C)C(=O)O)sc3c(Cl)c1O)CN(C(=O)C[C@H](C)C(=O)O)C2] | 670 |
| 155. | 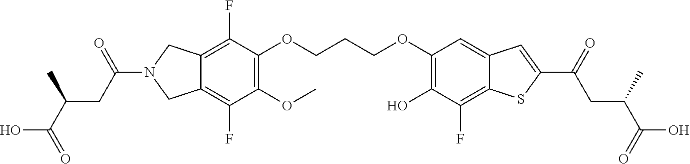 [SMILES: COc1c(F)c2c(c(F)c1OCCCOc1cc3cc(C(=O)C[C@H](C)C(=O)O)sc3c(F)c1O)CN(C(=O)C[C@H](C)C(=O)O)C2] | 654 |
| 156. | 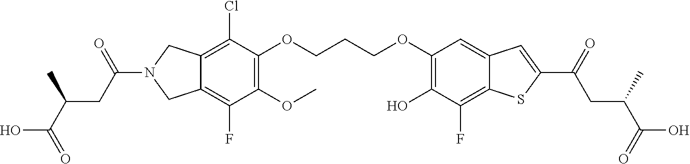 [SMILES: COc1c(F)c2c(c(Cl)c1OCCCOc1cc3cc(C(=O)C[C@H](C)C(=O)O)sc3c(F)c1O)CN(C(=O)C[C@H](C)C(=O)O)C2] | 670 |
| 157. | 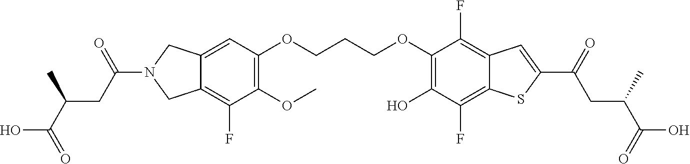 [SMILES: COc1c(OCCCOc2c(O)c(F)c3sc(C(=O)C[C@H](C)C(=O)O)cc3c2F)cc2c(c1F)CN(C(=O)C[C@H](C)C(=O)O)C2] | 654 |
| 158. | 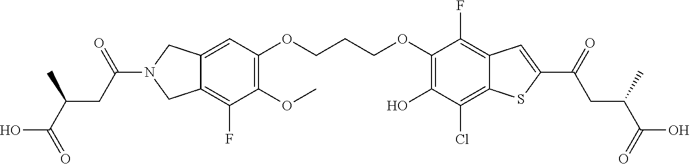 [SMILES: COc1c(OCCCOc2c(O)c(Cl)c3sc(C(=O)C[C@H](C)C(=O)O)cc3c2F)cc2c(c1F)CN(C(=O)C[C@H](C)C(=O)O)C2] | 670 |
| 159. | 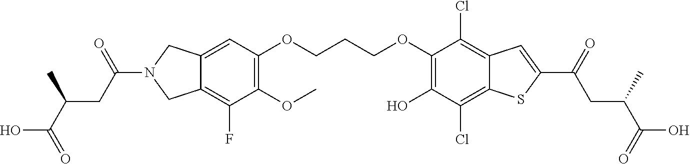 [SMILES: COc1c(OCCCOc2c(O)c(Cl)c3sc(C(=O)C[C@H](C)C(=O)O)cc3c2Cl)cc2c(c1F)CN(C(=O)C[C@H](C)C(=O)O)C2] | 686 |
| 160. | 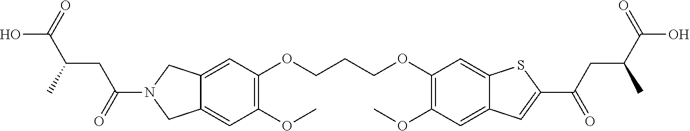 [SMILES: COc1cc2c(cc1OCCCOc1cc3sc(C(=O)C[C@H](C)C(=O)O)cc3cc1OC)CN(C(=O)C[C@H](C)C(=O)O)C2] | 614 |
| 161. | 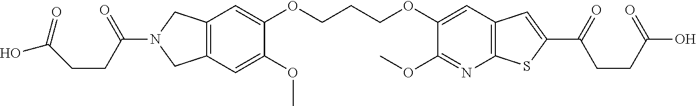 [SMILES: COc1cc2c(cc1OCCCOc1cc3cc(C(=O)CCC(=O)O)sc3nc1OC)CN(C(=O)CCC(=O)O)C2] | 587 |
| 162. | 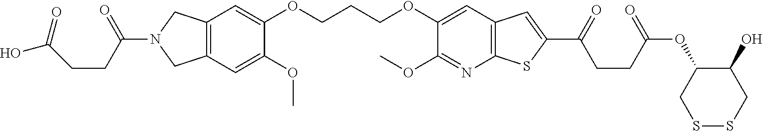 [SMILES: COc1cc2c(cc1OCCCOc1cc3cc(C(=O)CCC(=O)O[C@H]4CSSC[C@@H]4O)sc3nc1OC)CN(C(=O)CCC(=O)O)C2] | 721 |
| 163. | 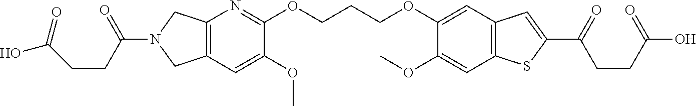 [SMILES: COc1cc2sc(C(=O)CCC(=O)O)cc2cc1OCCCOc1nc2c(cc1OC)CN(C(=O)CCC(=O)O)C2] | 587 |
| 164. | 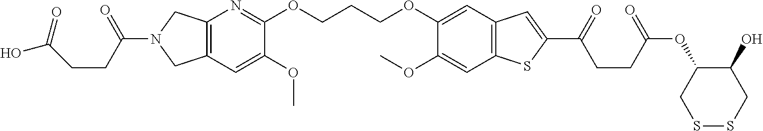 [SMILES: COc1cc2sc(C(=O)CCC(=O)O[C@H]3CSSC[C@@H]3O)cc2cc1OCCCOc1nc2c(cc1OC)CN(C(=O)CCC(=O)O)C2] | 721 |
| 165. | 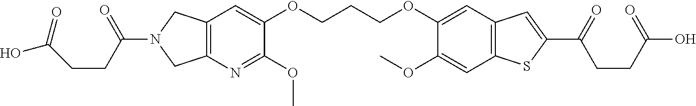 [SMILES: COc1cc2sc(C(=O)CCC(=O)O)cc2cc1OCCCOc1cc2c(nc1OC)CN(C(=O)CCC(=O)O)C2] | 587 |
| 166. | 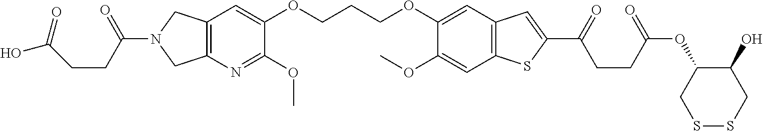 [SMILES: COc1cc2sc(C(=O)CCC(=O)O[C@H]3CSSC[C@@H]3O)cc2cc1OCCCOc1cc2c(nc1OC)CN(C(=O)CCC(=O)O)C2] | 721 |
| 167. | 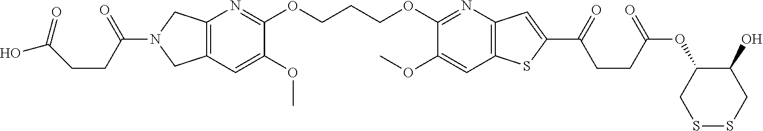 [SMILES: COc1cc2c(nc1OCCCOc1nc3cc(C(=O)CCC(=O)O[C@H]4CSSC[C@@H]4O)sc3cc1OC)CN(C(=O)CCC(=O)O)C2] | 722 |
| 168. | 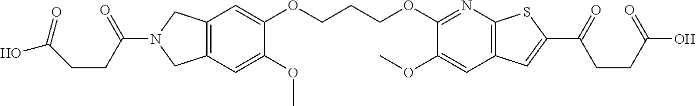 [SMILES: COc1cc2c(cc1OCCCOc1nc3sc(C(=O)CCC(=O)O)cc3cc1OC)CN(C(=O)CCC(=O)O)C2] | 587 |
| 169. | 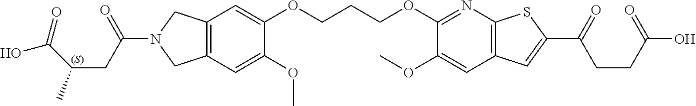 | 601 |
| 170. | 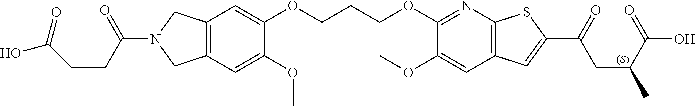 | 601 |
| 171. | 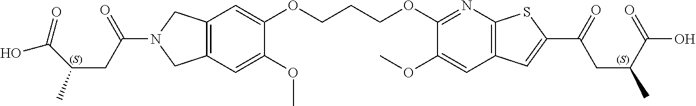 | 615 |
| 172. | 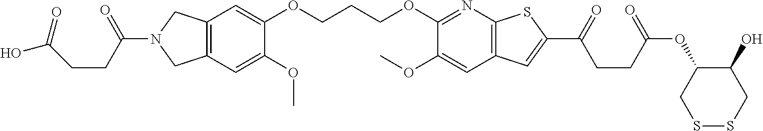 [SMILES: COc1cc2c(cc1OCCCOc1nc3sc(C(=O)CCC(=O)O[C@H]4CSSC[C@@H]4O)cc3cc1OC)CN(C(=O)CCC(=O)O)C2] | 721 |

### TESTING Example II: Human STING WT Binding Assay

Cisbio Bioassays' human STING WT binding assay (#64BDSTGPEG & 64BDSTGPEH, Cisbio) is for quantitative measurement of human STING WT ligand using HTRF® technology.

### 1. Adding Compounds

Negative control: Dispense 5 μL of diluent into each negative control well. Standard: Dispense 5 μL of each Human STING WT Standard 2′3′-cGAMP (Std 0—Std 7) into each standard well. Compound: Dispense 5 μL of compound into each compound well.

### 2. Adding Proteins

Negative control: Add 5 μL of detection buffer to all wells. Other wells: Add 5 μL of human STING WT protein 6His-tagged protein to all wells.

### 3. Adding Antibodies

Add 10 μL of premixed STING WT ligand d2 reagent and 6His T_{b} antibody working solution to all wells.

### 4. RT Incubation

Seal the plate and incubate 3 hours at RT or at Over Night if necessary.

### 5. Reading Plate

Remove the plate sealer and read on an HTRF® compatible reader (PerkinElmer, USA). Results were analyzed with a two-wavelength signal ratio: intensity (665 nm)/intensity (620 nm).

### 6. Curve Fitting

Calculate HTRF Ratio:

$$Ratio = \frac{Signal 665 nm}{Signal 620 nm} \times {10}^{4}$$

Fit the data in GraphPad to obtain IC₅₀ values using equation (2)

Y=Bottom+(Top-Bottom)/(1+10\hat{ }((LogIC₅₀—X)*Hill Slope)) Equation (2):
Y is HTRF Ratio and X is compound concentration.
IC₅₀ value of binding assay for human STING WT:

| Example | hSTING WT binding IC₅₀/nM |
|---|---|
| II-1 | 0.06 |
| II-2 | 11.37 |
| II-3 | 8.40 |
| II-4 | 0.18 |
| II-5 | 0.32 |
| II-7 | 0.36 |
| II-8 | 1.04 |
| II-9 | 0.65 |
| II-9 | 2.23 |
| II-10 | 0.28 |
| II-11 | 2.54 |
| II-13 | 12.84 |
| II-14 | 46.60 |
| II-15 | 92.57 |
| II-16 | >150 |
| II-17 | 0.19 |
| II-18 | 4.59 |
| II-19 | 54.62 |
| II-20 | 0.19 |
| II-21 | 158.60 |

### Linker Compounds

### Example III-1 Synthesis of Linker Compound M61-0

### Step a:

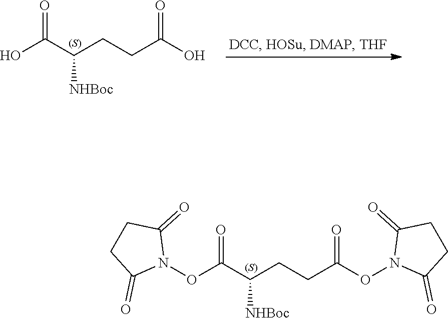

(2S)-2-[(tert-butoxycarbonyl) amino]pentanedioic acid (11.36 g, 45.946 mmol, 1 equiv), tetrahydrofuran (200 mL), N-hydroxysuccinimide (21.16 g, 183.784 mmol, 4 equiv) were added at room temperature. To the above mixture dicyclohexylcarbodiimide (18.96 g, 91.892 mmol, 2 equiv) was added at 0° C. The resulting mixture was stirred for lh at 0° C. Desired product could be detected by LCMS. The resulting mixture was filtered. The filter cake was washed with tetrahydrofuran (2×50 mL). The filtrate was used directly in the next step. LCMS (ES, m/z) [M-Boc+H]⁺=342.15.

### Step b:

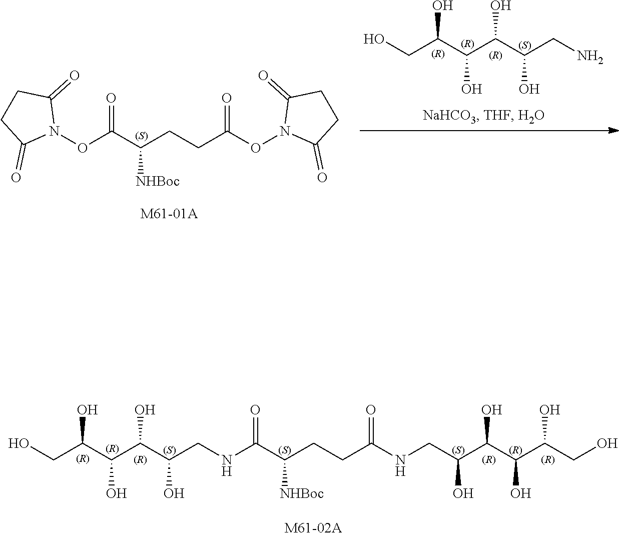

The filtrate of step a was added to the mixture of (2R,3R,4R,5S)-6-aminohexane-1,2,3,4,5-pentol (25.00 g, 137.98 mmol, 3.00 equiv.) and sodium bicarbonate (7.73 g, 91.99 mmol, 3.58 mL, 2.00 equiv.) in water (200 mL) at 20° C. The reaction mixture was stirred at 20° C. for 2 hours. The reaction mixture was concentrated to remove THF and the residue was purified by reverse flash chromatography with the following conditions: column, C18 silica gel; mobile phase, MeCN in water, 0% to 10% gradient in 20 min; detector, UV 254 nm. There was tert-butyl N-[(1S)-1,3-bis({[(2S,3R,4R,5R)-2,3,4,5,6-pentahydroxyhexyl]carbamoyl}) propyl]carbamate (12.8 g, 48.3%) obtained as a off-white solid. LCMS (ES, m/z): [M+H]⁺=574.40. ¹H NMR (400 MHz, DMSO-d₆) δ 7.72 (t, J=5.6 Hz, 11.2 Hz, 1H), 7.65 (t, J=4.8 Hz, 11.2 Hz, 1H), 6.90 (d, J=8.0 Hz, 1H), 4.76 (d, J=4.6 Hz, 2H), 4.48 (dd, J=5.6, 1.8 Hz, 2H), 4.44-4.22 (m, 6H), 3.90-3.85 (m, 1H), 3.72-3.52 (m, 6H), 3.50-3.44 (m, 2H), 3.43-3.30 (m, 4H), 3.28-3.22 (m 1H), 3.10-2.94 (m, 2H), 2.21-2.04 (m, 2H), 1.89-1.77 (m, 1H), 1.71-1.67 (m, 1H), 1.38 (s, 9H).

### Step c:

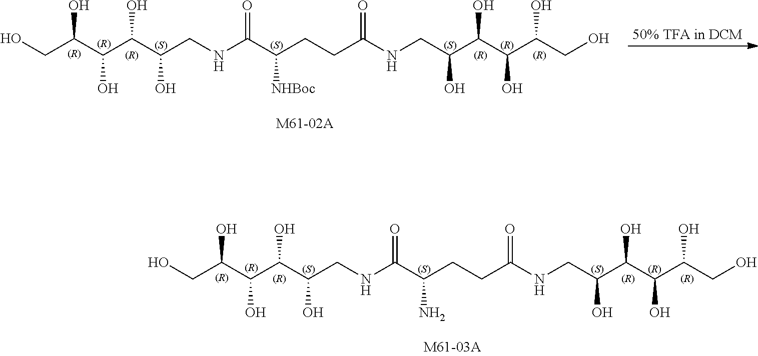

Tert-butyl N-[(1S)-1,3-bis({[(2S,3R,4R,5R)-2,3,4,5,6-pentahydroxyhexyl]carbamoyl}) propyl]carbamate (6.5 g, 11.332 mmol, 1 equiv), dichloromethane (50 mL) and TFA (50 mL) were added at room temperature. The resulting mixture was stirred for lh at room temperature. Desired product could be detected by LCMS. The mixture was basified to pH=8 with anion exchanger resin. The resulting mixture was filtered, and the resin was washed with water (2×20 mL). The filtrate was lyophilized. There was (2S)-2-amino-N, N-bis[(2S,3R,4R,5R)-2,3,4,5,6-pentahydroxyhexyl]pentanediamide (4.6 g, 85.73%) obtained as a light yellow solid. LCMS (ES, m/z): [M+H]⁺=474.25. ¹H NMR (400 MHz, DMSO-d₆) δ 8.09 (t, J=5.7 Hz, 1H), 7.83 (t, J=5.7 Hz, 1H), 3.66-3.53 (m, 7H), 3.53-3.43 (m, 3H), 3.38 (m, 6H), 3.36-3.31 (m, 2H), 3.06-3.00 (m, 2H), 2.34-2.09 (m, 2H), 1.89-1.82 (m, 1H), 1.73-1.68 (m, 1H), 1.13-1.03 (m, 1H).

### Step d:

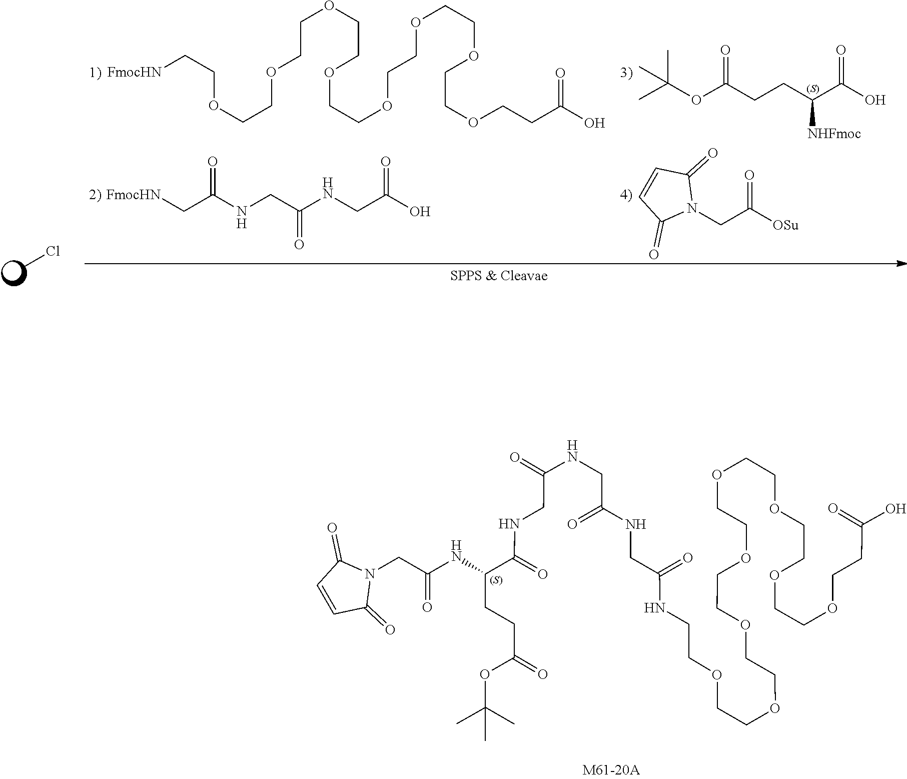

Trt-resin (3.0 g, 3.3 mmol, 1.00 equiv) and dichloromethane (45 mL) were added at room temperature. The Trt-Cl resin (3.0 g, 3.3 mmol) was swelling for 30 min. After filtration, the Trt-Cl resin was washed with N, N-dimethylformamide (3×45 mL). Then N, N-dimethylformamide (60 mL), 1-{[(9H-fluoren-9-ylmethoxy) carbonyl]amino}-3,6,9,12,15,18,21,24-octaoxaheptacosan-27-oic acid (4.53 g, 6.822 mmol, 0.71 equiv) and N, N-diisopropylethylamine (8.5 g, 33.0 mmol, 10 equiv) were added in sequence. Then the column was bubbled with nitrogen atmosphere for 2 h. After filtration, the residue was washed with N,N-dimethylformamide (3×45 mL), 60 mL dichloromethane/methanol/N, N-diisopropylethylamine (80%/15%/5%) for 20 min, then dichloromethane (3×45 mL). The residue was dried by nitrogen atmosphere for 2h. To the above residue 20 mL 20% piperdine in N, N-dimethylformamide was added at room temperature. The resulting mixture was stirred for additional lh at room temperature. After filtration, the residue was washed with N, N-dimethylformamide (3×45 mL) and dichloromethane (3×45 mL). The residue was dried by nitrogen atmosphere for 2h. ther was 4-{[(1-amino-3,6,9,12,15,18,21,24-octaoxaheptacosanoyl) oxy]-Trt resin (4.4 g, 63.84%) obtained as a yellow powder. The product was used directly in the next step. 4-{[(1-amino-3,6,9,12,15,18,21,24-octaoxaheptacosanoyl) oxy]-Trt resin (2.0 g, 2.788 mmol, 1 equiv) and dichloromethane (45 mL) were added at room temperature. The Trt-Cl resin (500 mg, 0.45 mmol) was swelling for 30 min. After filtration, the resin was washed with N,N-dimethylformamide (3×40 mL). Then[2-(2-{[(9H-fluoren-9-ylmethoxy) carbonyl]amino}acetamido) acetamido]acetic acid (5.74 g, 13.940 mmol, 5 equiv) N, N-dimethylformamide (50 mL), N, N-diisopropylethylamine (3.60 g, 27.880 mmol, 10 equiv) and 2-(7-Azabenzotriazol-1-yl)-N, N, N′, N′-tetramethyluronium hexafluorophosphate (5.30 g, 13.940 mmol, 5 equiv) were added in sequence. Then the column was bubbled with nitrogen atmosphere for 2 h. After filtration, the residue was washed with N, N-dimethylformamide (3×40 mL), dichloromethane (3×40 mL). The residue was dried by nitrogen atmosphere for 2h. To the above residue, 50 mL 20% piperdine in N, N-dimethylformamide was added at room temperature. The resulting mixture was stirred for additional lh at room temperature. After filtration, the residue was washed with N, N-dimethylformamide (3×15 mL) and dichloromethane (3×15 mL). there was (4-{[(1-{2-[2-(2-aminoacetamido) acetamido]acetamido}-3,6,9,12,15,18,21,24-octaoxaheptacosanoyl) oxy]-Trt-resin obtained as a yellow powder. The residue was dried by nitrogen atmosphere for 2h, and used in the next step directly. (4-{[(1-{2-[2-(2-aminoacetamido) acetamido]acetamido1-3,6,9,12,15,18,21,24-octaoxaheptacosanoyl) oxy]-Trt-resin (3 g, 2.091 mmol, 1 equiv) and dichloromethane (45 mL) were added at room temperature. The Trt-Cl resin (3 g, 2.7190 mmol) was swelling for 30 min. After filtration, the Trt-Cl resin was washed with N, N-Dimethylformamide (3×45 mL). Then (2S)-5-(tert-butoxy)-2-{[(9H-fluoren-9-yloxy) carbonyl]amino}-5-oxopentanoic acid (5.70 g, 13.850 mmol, 5 equiv), N, N-Dimethylformamide (10 mL), N,N-diisopropylethylamine (3.58 g, 27.700 mmol, 10 equiv) and 2-(7-Azabenzotriazol-1-yl)-N, N, N′, N′-tetramethyluronium hexafluorophosphate (5.27 g, 13.850 mmol, 5 equiv) were added in sequence. Then the column was bubbled with nitrogen atmosphere for 2 h. After filtration, the residue was washed with N, N-dimethylformamide (3×45 mL), dichloromethane (3×45 mL). The residue was dried by nitrogen atmosphere for 2 h. Then 20 mL 20% piperidine in N, N-Dimethylformamide was added and bubbled with nitrogen atmosphere for lh. The residue was washed with N, N-dimethylformamide (3×40 mL). There was 1-[2-(2-{2-[(2S)-5-(tert-butoxy)-2-{[(9H-fluoren-9-yloxy) carbonyl]amino}-5-oxopentanamido]acetamido}acetamido) acetamido]-3,6,9,12,15,18,21,24-octaoxaheptacosan-27-oic-Trt-resin obtained as a yellow powder. Then the residue was directly used in the next step. 1-[2-(2-{2-[(2S)-5-(tert-butoxy)-2-{[(9H-fluoren-9-yloxy) carbonyl]amino}-5-oxopentanamido]acetamido}acetamido) acetamido]-3,6,9,12,15,18,21,24-octaoxaheptacosan-27-oic-Trt-resin (1.5 g, 2.091 mmol, 1 equiv) and dichloromethane (15 mL) were added at room temperature. The Trt resin (450 mg, 0.45 mmol) was swelling for 30 min. After filtration, the resin was washed with N, N-dimethylformamide (3×15 mL). Then (2-chlorophenyl) (4-methylphenyl) phenylmethyl 1-[2-(2-{2-[(2S)-2-amino-5-(tert-butoxy)-5-oxopentanamido]acetamido}acetamido) acetamido]-3,6,9,12,15,18,21,24-octaoxaheptacosan-27-oate (2.96 g, 2.719 mmol, 1 equiv), N, N-dimethylformamide (30 mL) and trimethylamine (1757.03 mg, 13.595 mmol, 5 equiv) were added in sequence.

Then the column was bubbled with nitrogen atmosphere for 2 h. After filtration, the residue was washed with N, N-dimethylformamide (3×15 mL) and dichloromethane (3×15 mL). The residue was dried by nitrogen atmosphere for 2 h. Then 40 mL 20% hexafluoroisopropanol in dichloromethane was added and bubbled with nitrogen atmosphere for lh. The filtrate was concentrated under reduced pressure. There was 1-[2-(2-{2-[(2S)-5-(tert-butoxy)-2-[2-(2,5-dioxopyrrol-1-yl) acetamido]-5-oxopentanamido]acetamido}acetamido) acetamido]-3,6,9,12,15,18,21,24-octaoxaheptacosan-27-oic acid (350 mg, 13.77%) obtained as a colorless oil.

LCMS: [M+H]⁺=935.60. ¹H NMR (400 MHz, DMSO-d₆) δ 12.15 (s, 1H), 8.42 (d, J=8.0 Hz, 1H), 8.27 (t, J=2.0 Hz, 11.6 Hz, 1H), 8.15-8.09 (m, 2H), 8.05 (d, J=7.5 Hz, 6H), 7.86 (t, J=5.6 Hz, 11.2 Hz, 1H), 7.10 (s, 1H), 5.76 (s, 2H), 5.25-5.08 (m, 6H), 4.34-4.24 (m, 1H), 4.18-4.04 (m, 2H), 3.83-3.65 (m, 7H), 3.59 (d, J=12.7 Hz, 3H), 3.40 (t, J=6.0 Hz, 3H), 3.21 (q, J=5.8 Hz, 2H), 2.44 (t, J=6.3 Hz, 3H), 2.22 (t, J=8.1 Hz, 2H), 1.90-1.87 (m, 1H), 1.75-1.68 (m, 1H), 1.39 (s, 9H).

### Step e:

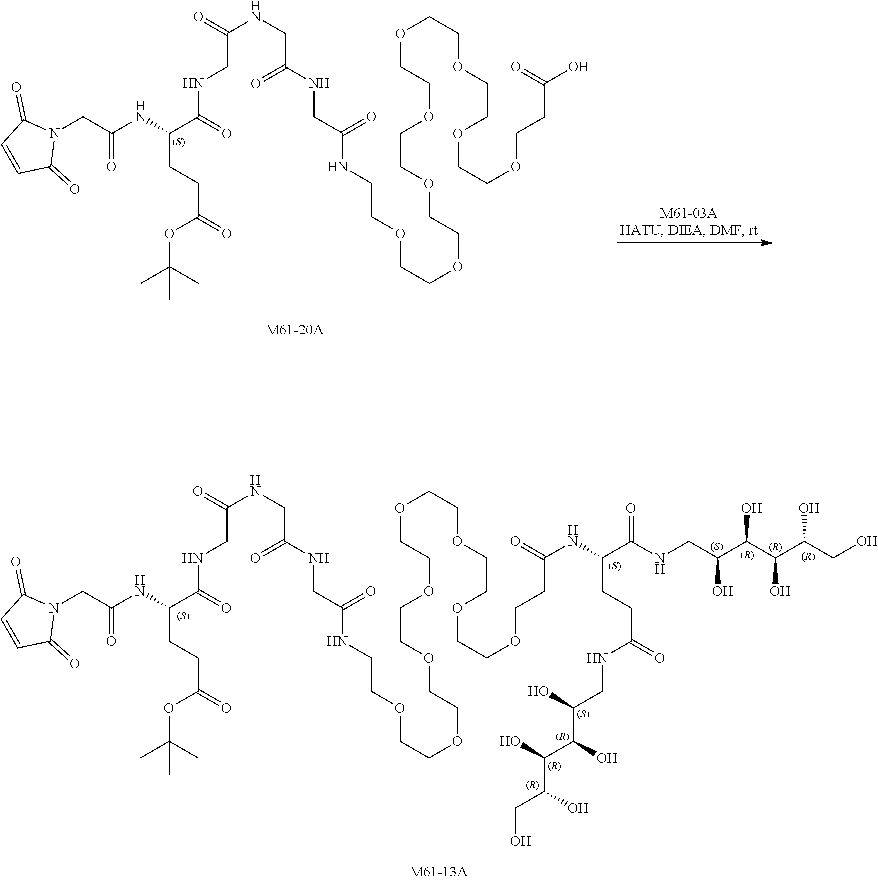

1-[2-(2-{2-[(2S)-2-amino-5-(tert-butoxy)-5-oxopentanamido]acetamido}acetamido) acetamido]-3,6,9,12,15,18,21,24-octaoxaheptacosan-27-oic acid (1.0 g, 1.253 mmol, 1 equiv), N(S)-2-amino-N1,N5-bis((2S,3R,4R,5R)-2,3,4,5,6-pentahydroxyhexyl)pentanediamide (711.91 mg, 1.504 mmol, 1.2 equiv), N-diisopropylethylamine (647.94 mg, 5.012 mmol, 4 equiv) and N, N-Dimethylformamide (15 mL) were added at room temperature. To the above mixture 2-(7-Azabenzotriazol-1-yl)-N, N, N′, N′-tetramethyluronium hexafluorophosphate (953.09 mg, 2.506 mmol, 2 equiv) was added at room temperature. The resulting mixture was stirred for additional 2h at room temperature. The residue was purified by reverse flash chromatography with the following conditions: column, C18 gel; mobile phase, acetonitrile in water (0.1% TFA), 0% to 50% gradient in 30 min; detector, UV 254 nm. There was tert-butyl (4S)-4-[({[({[(26-{[(1S)-1,3-bis({[(2S,3R,4R,5R)-2,3,4,5,6-pentahydroxyhexyl]carbamoyl}) propyl]carbamoyl}-3,6,9,12,15,18,21,24-octaoxahexacosan-1-yl) carbamoyl]methyl}carbamoyl) methyl]carbamoyl}methyl) carbamoyl]-4-[2-(2,5-dioxopyrrol-1-yl) acetamido]butanoate (400 mg, 22.95%) obtained as a colorless oil. LCMS (ES, m/z): [M+2H]²=696.15. ¹HNMR (300 MHz, DMSO-d₆) δ 8.41 (d, J=8.0 Hz, 1H), 8.26 (s, 1H), 8.17-8.01 (m, 3H), 7.84 (s, 1H), 7.71 (d, J=15.1 Hz, 2H), 7.09 (d, J=1.7 Hz, 2H), 4.27 (d, J=24.5 Hz, 3H), 4.15-4.08 (m, 2H), 3.74 (s, 4H), 3.72-3.53 (m, 11H), 3.31-3.11 (m, 5H), 3.03 (d, J=8.4 Hz, 1H), 2.40 (s, 2H), 2.28-2.17 (m, 2H), 2.09 (d, J=8.6 Hz, 1H), 1.89 (s, 1H), 1.71 (s, 1H), 1.39 (d, J=1.7 Hz, 5H), 1.12 (t, J=3.5 Hz, 2H).

### Step f:

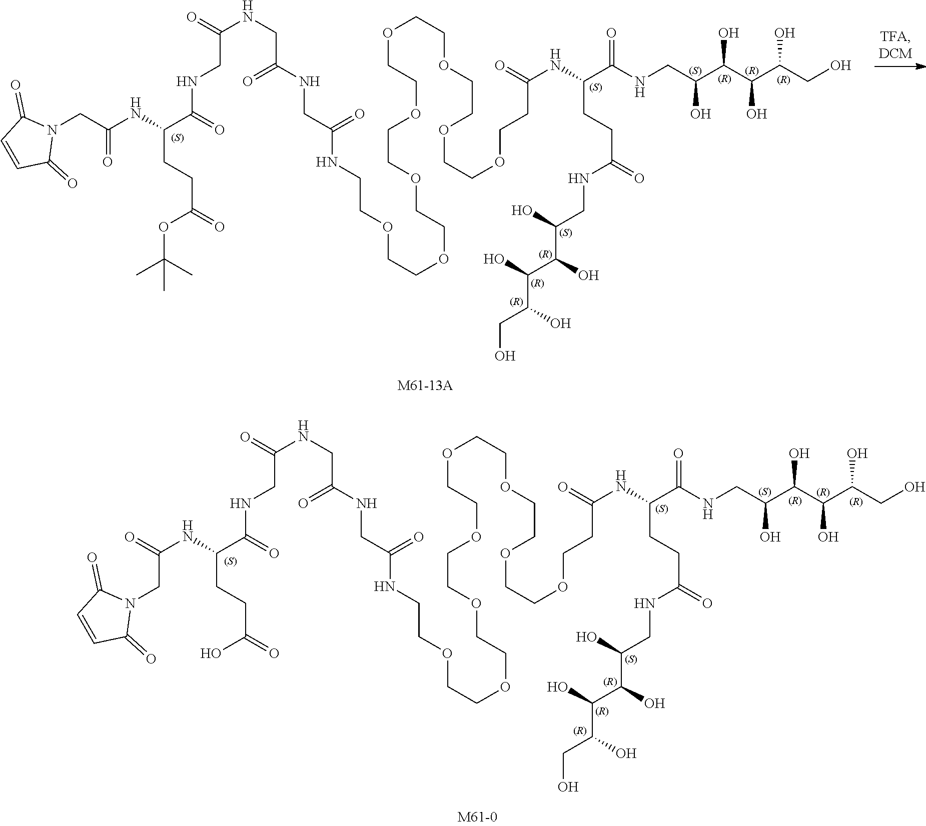

Tert-butyl (4S)-4-[({[({[(26-{[(1S)-1,3-bis({[(2S,3R,4R,5R)-2,3,4,5,6-pentahydroxyhexyl]carbamoyl}) propyl]carbamoyl}-3,6,9,12,15,18,21,24-octaoxahexacosan-1-yl) carbamoyl]methyl}carbamoyl) methyl]carbamoyl}methyl) carbamoyl]-4-[2-(2,5-dioxopyrrol-1-yl)acetamido]butanoate (350 mg, 0.252 mmol, 1 equiv) and dichloromethane (6 mL) were added at room temperature. To the above mixture TFA (6 mL) was added dropwise at room temperature. The resulting mixture was stirred for additional lh at room temperature.

Desired product was detected by LCMS. The resulting mixture was concentrated under reduced pressure. The residue was purified by Prep-HPLC with the following conditions (Column: XBridge Prep OBD C18 Column, 30*150 mm, 5 μm ; Mobile Phase A: water(0.05% TFA ), Mobile Phase B: ACN; Flow rate: 60 mL/min; Gradient: 3% B to 15% B in 9 min, 15% B; Wave Length: 220 nm; RT1(min): 7; Number Of Runs: 0) to obtain (4S)-4-[({[({[(26-{[(1S)-1,3-bis({[(2S,3R,4R,5R)-2,3,4,5,6-pentahydroxyhexyl]carbamoyl})propyl]carbamoyl}-3,6,9,12,15,18,21,24-octaoxahexacosan-1-yl)carbamoyl]methyl}carbamoyl)methyl]carbamoylmethyl)carbamoyl]-4-[2-(2,5-dioxopyrrol-1-yl)acetamido]butanoic acid (110.7 mg, 31.48%) as a colorless semi-solid. LCMS [M+H]⁺=1332.75. ¹H NMR (400 MHz, Deuterium Oxide) 6 6.84 (s, 2H), 4.35-4.16 (m, 4H), 3.95-3.83 (m, 4H), 3.82 (s, 2H), 3.78-3.74 (m, 2H), 3.73-3.62 (m, 8H), 3.59-3.57 (m, 31H), 3.55-3.50 (m, 3H), 3.41-3.27 (m, 4H), 3.20-3.14 (m, 2H), 2.54-2.47 (m, 2H), 2.45-2.38 (m, 2H), 2.26 (t, J=8.0, 15.6 Hz, 2H), 2.13-1.95 (m, 2H), 1.95-1.79 (m, 2H).

### Example III-2 Synthesis of Linker Compound M66-0

### Step a:

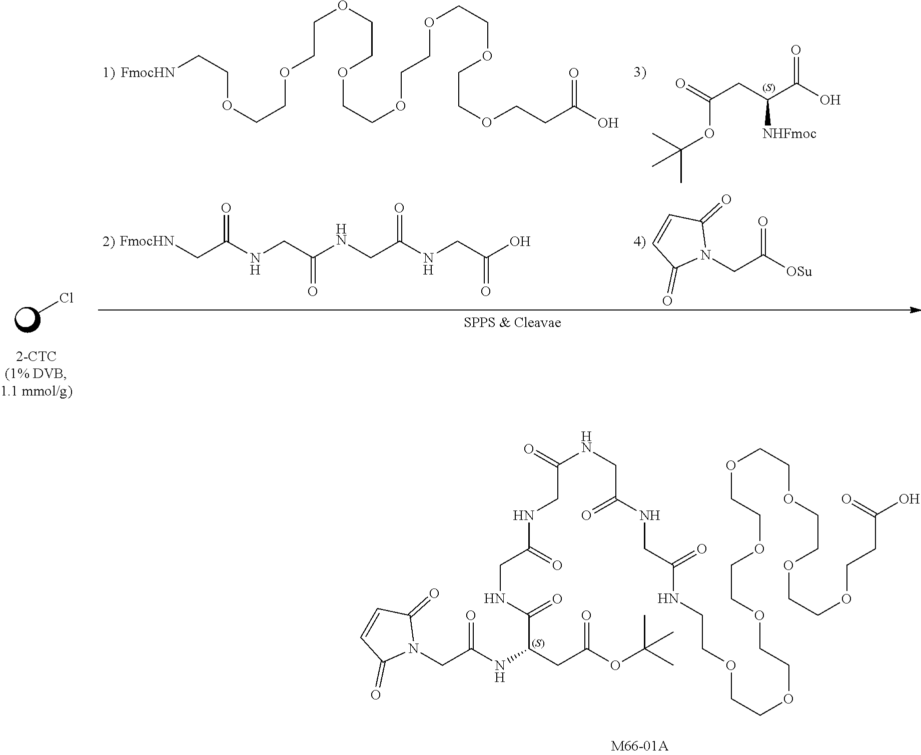

Trt-resin (3.0 g, 3.3 mmol, 1.00 equiv) and dichloromethane (45 mL) were added at room temperature. The Trt-Cl resin (3.0 g, 3.3 mmol) was swelling for 30 min. After filtration, the Trt-Cl resin was washed with N, N-dimethylformamide (3×45 mL). Then N, N-dimethylformamide (60 mL), 1-{[(9H-fluoren-9-ylmethoxy) carbonyl]amino}-3,6,9,12,15,18,21,24-octaoxaheptacosan-27-oic acid (4.53 g, 6.822 mmol, 0.71 equiv) and N, N-diisopropylethylamine (8.5 g, 33.0 mmol, 10 equiv) were added in sequence. Then the column was bubbled with nitrogen atmosphere for 2 h. After filtration, the residue was washed with N, N-dimethylformamide (3×45 mL), 60 mL dichloromethane/methanol/N, N-diisopropylethylamine (80%/15%/5%) for 20 min, then dichloromethane (3×45 mL). The residue was dried by nitrogen atmosphere for 2h. To the above residue 20 mL 20% piperdine in N, N-dimethylformamide was added at room temperature. The resulting mixture was stirred for additional 1h at room temperature. After filtration, the residue was washed with N, N-dimethylformamide (3×45 mL) and dichloromethane (3×45 mL). The residue was dried by nitrogen atmosphere for 2 h. There was 4-{[(1-amino-3,6,9,12,15,18,21,24-octaoxaheptacosanoyl) oxy]-Trt resin (4.4 g, 63.84%) obtained as a yellow powder. The product was used directly in the next step. 4-{[(1-amino-3,6,9,12,15,18,21,24-octaoxaheptacosanoyl) oxy]-Trt resin (2.0 g, 2.788 mmol, 1 equiv) and dichloromethane (45 mL) were added at room temperature. The resin (500 mg, 0.45 mmol) was swelling for 30 min. After filtration, the resin was washed with N, N-dimethylformamide (3×40 mL). Then[2-(2-{[(9H-fluoren-9-ylmethoxy) carbonyl]amino}acetamido) acetamido]acetic acid (5.74 g, 13.940 mmol, 5 equiv) N, N-dimethylformamide (50 mL), N, N-diisopropylethylamine (3.60 g, 27.880 mmol, 10 equiv) and 2-(7-Azabenzotriazol-1-yl)-N, N, N′, N′-tetramethyluronium hexafluorophosphate (5.30 g, 13.940 mmol, 5 equiv) were added in sequence. Then the column was bubbled with nitrogen atmosphere for 2 h. After filtration, the residue was washed with N, N-Dimethylformamide (3×40 mL) and dichloromethane (3×40 mL). The residue was dried by nitrogen atmosphere for 2h. To the above residue 50 mL 20% piperdine in N, N-dimethylformamide was added at room temperature. The resulting mixture was stirred for additional lh at room temperature. After filtration, the residue was washed with N, N-dimethylformamide (3×15 mL) and dichloromethane (3×15 mL). There was (4-{[(1-{2-[2-(2-aminoacetamido) acetamido]acetamido}-3,6,9,12,15,18,21,24-octaoxaheptacosanoyl) oxy]-Trt-resin obtained as a yellow powder. The residue was dried by nitrogen atmosphere for 2h, and used in the next step directly. 4-({[1-(2-{2-[2-(2-aminoacetamido) acetamido]acetamido}acetamido)-3,6,9,12,15,18,21,24-octaoxaheptacosanoyl]oxy}Trt-resin (4.8 g, 5.077 mmol, 1 equiv) and dichloromethane (50 mL) were added at room temperature. The resin was swelling for 30 min. After filtration, the resin was washed with N, N-dimethylformamide (3×50 mL). Then (2S)-5-(tert-butoxy)-2-{[(9H-fluoren-9-yl methoxy) carbonyl]amino}-5-oxopentanoic acid (8.64 g, 20.308 mmol, 4 equiv), N, N-dimethylformamide (50 mL), N, N-diisopropylethylamine (5.25 g, 40.616 mmol, 8 equiv) and 2-(7-Azabenzotriazol-1-yl)-N, N, N′, N′-tetramethyluronium hexafluorophosphate (7.72 g, 20.308 mmol, 4 equiv) were added in sequence. Then the column was bubbled with nitrogen atmosphere for 2 h. After filtration, the residue was washed with N, N-dimethylformamide (3×50 mL) and dichloromethane (3×50 mL). The residue was dried by nitrogen atmosphere for 2h. To the above residue 50 mL 20% piperdine in N, N-dimethylformamide was added at room temperature. The resulting mixture was stirred for additional lh at room temperature. After filtration, the residue was washed with N, N-dimethylformamide (3×15 mL) and dichloromethane (3×15 mL). There was 4-{[(1-{2-[2-(2-{2-[(2S)-2-amino-5-(tert-butoxy)-5-oxopentanamido]acetamido}acetamido) acetamido]acetamido}-3,6,9,12,15,18,21,24-octaoxaheptacosanoyl) oxy]Trt-resin obtained as a yellow powder. The residue was dried by nitrogen atmosphere for 2h, and used in the next step directly.

4-{[(1-{2-[2-(2-{2-[(2S)-2-amino-5-(tert-butoxy)-5-oxopentanamido]acetamido}acetamido) acetamido]acetamido}-3,6,9,12,15,18,21,24-octaoxaheptacosanoyl) oxy]Trt resin (5.8 g, 5.130 mmol, 1 equiv) and dichloromethane (20 mL) were added at room temperature. The resin was swelling for 30 min. After filtration, the resin was washed with N, N-dimethylformamide (3×20 mL). Then 2,5-dioxopyrrolidin-1-yl 2-(2,5-dioxopyrrol-1-yl) acetate (1.29 g, 5.130 mmol, 1 equiv), N, N-dimethylformamide (20 mL), and trimethylamine (1.56 g, 15.390 mmol, 3 equiv) were added in sequence. Then the column was bubbled with nitrogen atmosphere for 2 h. After filtration, the residue was washed with N, N-dimethylformamide (3×20 mL) and dichloromethane (3×20 mL). The residue was dried by nitrogen atmosphere for 2 h. Then 20 mL 20% hexafluoroisopropanol in dichloromethane (20 ml) was added and bubbled with nitrogen atmosphere for 2h. There was 1-{2-[2-(2-{2-[(2S)-4-(tert-butoxy)-2-[2-(2,5-dioxopyrrol-1-yl) acetamido]-4-oxobutanamido]acetamido}acetamido)acetamido]acetamido}-3,6,9,12,15,18,21,24-octaoxaheptacosan-27-oic acid (350 mg, 13.77%) obtained as a colorless oil. LCMS (ES, m/z): [M+H]⁺=978.60. ¹H NMR (400 MHz, DMSO-d₆) δ 8.55 (d, J=8.2 Hz, 1H), 8.27 (t, J=5.8 Hz, 1H), 8.14-8.02 (m, 12, H), 7.86 (t, J=5.5 Hz, 1H), 7.10 (s, 2H), 5.76 (s, 1H), 5.16 (m, 7H), 4.68-4.58 (m, 1H), 4.15 (d, J=16.9 Hz, 1H), 4.03 (d, J=16.9 Hz, 1H), 3.77-3.65 (m, 12H), 3.59 (t, J=6.4 Hz, 4H), 3.40 (t, J=5.9 Hz, 4H), 3.33 (s, 13H), 3.21 (q, J=5.9 Hz, 3H), 2.69 (dd, J=15.8, 5.2 Hz, 2H), 2.47-2.37 (m, 6H), 1.38 (s, 11H).

### Step b:

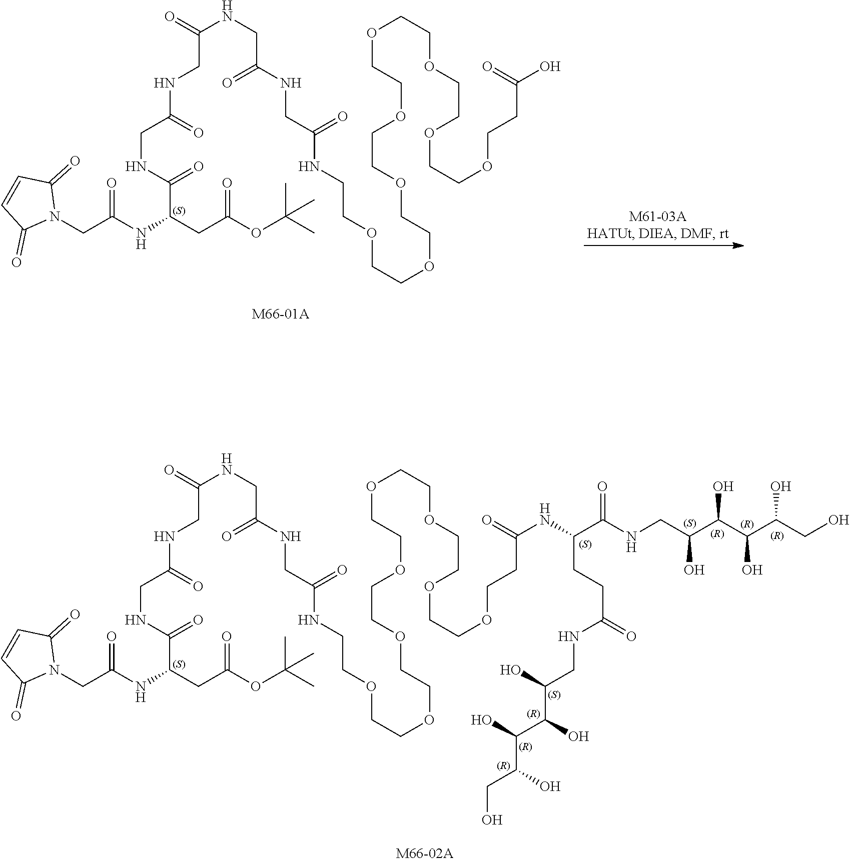

1-{2-[2-(2-{2-[(2S)-4-(tert-butoxy)-2-[(tert-butoxycarbonyl) amino]-4-oxobutanamido]acetamido}acetamido)acetamido]acetamido}-3,6,9,12,15,18,21,24-octaoxaheptacosan-27-oic acid (500 mg, 0.531 mmol, 1 equiv), (2S)-2-amino-N,N′-bis[(2S,3R,4R,5R)-2,3,4,5,6-pentahydroxyhexyl]pentanediamide (503.14 mg, 1.062 mmol, 2 equiv), N, N-diisopropylethylamine (549.38 mg, 4.248 mmol, 8 equiv), and N, N-dimethylformamide (15 mL) were added at room temperature. To the above mixture 2-(7-Azabenzotriazol-1-yl)-N, N, N′, N′-tetramethyluronium hexafluorophosphate (808.12 mg, 2.124 mmol, 4 equiv) was added at room temperature. The resulting mixture was stirred for additional 2h at room temperature. The residue was purified by reverse flash chromatography with the following conditions: column, C18 silica gel; mobile phase, acetonitrile in water (0.1% TFA), 0% to 50% gradient in 30 min; detector, UV 254 nm. There was tert-butyl (4S)-4-[({[({[(26-{[(1S)-1,3-bis({[(2S,3R,4R,5R)-2,3,4,5,6-pentahydroxyhexyl]carbamoyl}) propyl]carbamoyl}-3,6,9,12,15,18,21,24-octaoxahexacosan-1-yl) carbamoyl]methyl}carbamoyl) methyl]carbamoyl}methyl) carbamoyl]-4-[2-(2,5-dioxopyrrol-1-yl) acetamido]butanoate (400 mg, 22.95%) obtained as a colorless oil. LCMS (ES, m/z): [M+2H]²=717.70. ¹H NMR (400 MHz, DMSO-d₆) δ 8.55 (d, J=8.2 Hz, 1H), 8.27 (t, J=5.8 Hz, 1H), 8.13-8.03 (m, 4H), 7.86 (t, J=5.7 Hz, 1H), 7.76-7.68 (m, 2H), 7.10 (s, 2H), 4.65-4.60 (m, 8H), 4.28-4.01 (m, 4H), 3.83-3.65 (m, 6H), 3.64-3.53 (m, 11H), 3.50 (s, 7H), 3.49-3.35 (m, 7H), 3.35-3.17 (m, 5H), 3.14 (s, 2H), 3.06-2.99 (m, 2H), 2.74-2.62 (m, 2H), 2.50-2.38 (m, 5H), 2.09 (t, J=7.6 Hz, 2H), 1.89-1.84 (m, 1H), 1.75-1.67 (m, 1H), 1.38 (s, 9H).

### Step c:

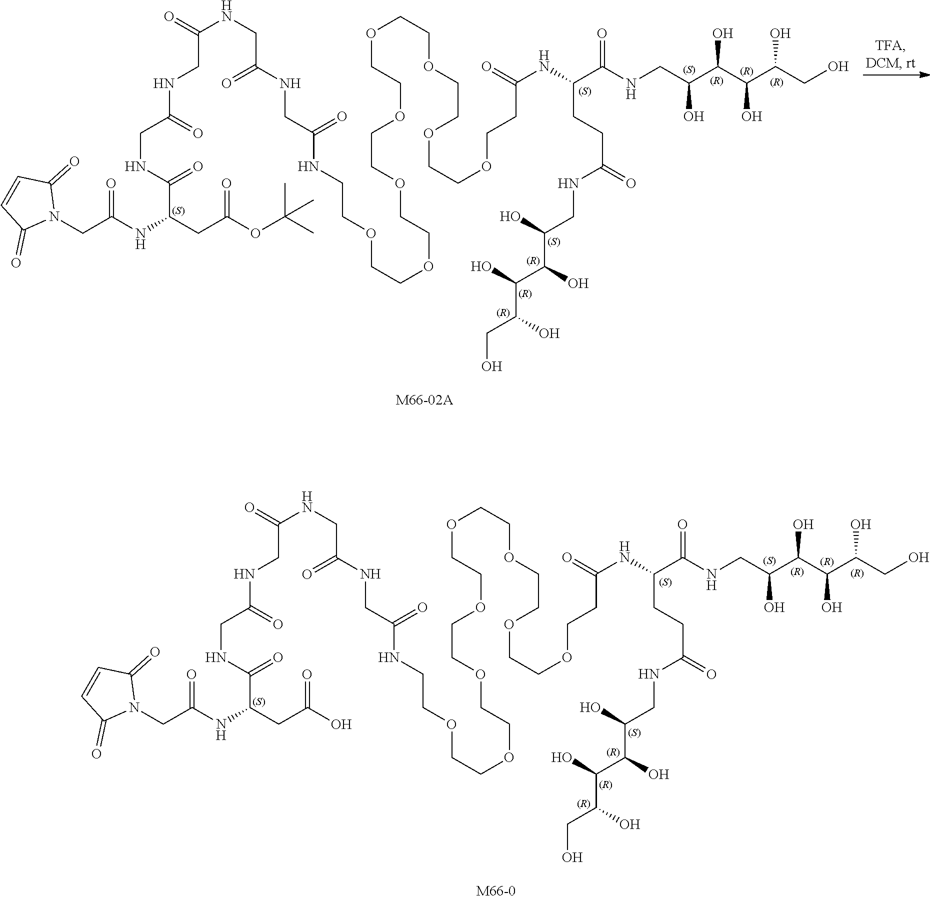

Tert-butyl (3S)-3-({[({[({[(26-{[(1S)-1,3-bis({[(2S,3R,4R,5R)-2,3,4,5,6-pentahydroxyhexyl]carbamoyl})propyl]carbamoyl}-3,6,9,12,15,18,21,24-octaoxahexacosan-1-yl)carbamoyl]methyl}carbamoyl)methyl]carbamoyl}methyl)carbamoyl]methyl}carbamoyl)-3-[2-(2,5-dioxopyrrol-1-yl)acetamido]propanoate (100 mg, 0.070 mmol, 1 equiv) and DCM (10 mL, 157.306 mmol, 250.55 equiv) were added at room temperature. To the above mixture TFA (10 mL, 134.631 mmol, 214.43 equiv) was added dropwise at room temperature. The resulting mixture was stirred for additional 40 min at room temperature. Desired product was detected by LCMS. The resulting mixture was concentrated under reduced pressure. The reaction was quenched by the addition of Water (5 mL) at room temperature. The crude product was purified by Prep-HPLC with the following conditions (Column: XBridge Prep OBD C18 Column, 30*150 mm, 5 μm; Mobile Phase A: water(0.05% TFA ), Mobile Phase B: ACN; Flow rate: 60 mL/min; Gradient: 3% B to 15% B in 9 min, 15% B; Wave Length: 220 nm; RT1(min): 7; Number Of Runs: 0) to obtain (3S)-3-({[({[({[(26-{[(1S)-1,3-bis({[(2S,3R,4R,5R)-2,3,4,5,6-pentahydroxyhexyl]carbamoyl})propyl]carbamoyl}-3,6,9,12,15,18,21,24-octaoxahexacosan-1-yl)carbamoyl]methyl}carbamoyl)methyl]carbamoyl}methyl)carbamoyl]methyl}carbamoyl)-3-[2-(2,5-dioxopyrrol-1-yl)acetamido]propanoic acid (409.3 mg, 46.76%) as a colorless oil. LCMS (ES, m/z): [M+H]⁺=1359.70. ¹H NMR (400 MHz, Deuterium Oxide) 6 6.84 (s, 2H), 4.30-4.20 (m, 3H), 3.92-3.86 (m, 6H), 3.82 (m, 2H), 3.78-3.74 (m, 2H), 3.73-3.66 (m, 4H), 3.66-3.63 (m, 4H), 3.62-3.49 (m, 35H), 3.40-3.28 (m, 4H), 3.21-3.14 (m, 2H), 2.90-2.76 (m, H), 2.51-2.48 (m, 2H), 2.27 (t, J=7.7 Hz, 2H), 2.09-1.96 (m, 2H), 2.06-1.81 (m, 2H).

### Example II-3 Synthesis of linker compound M67-0

### Step a:

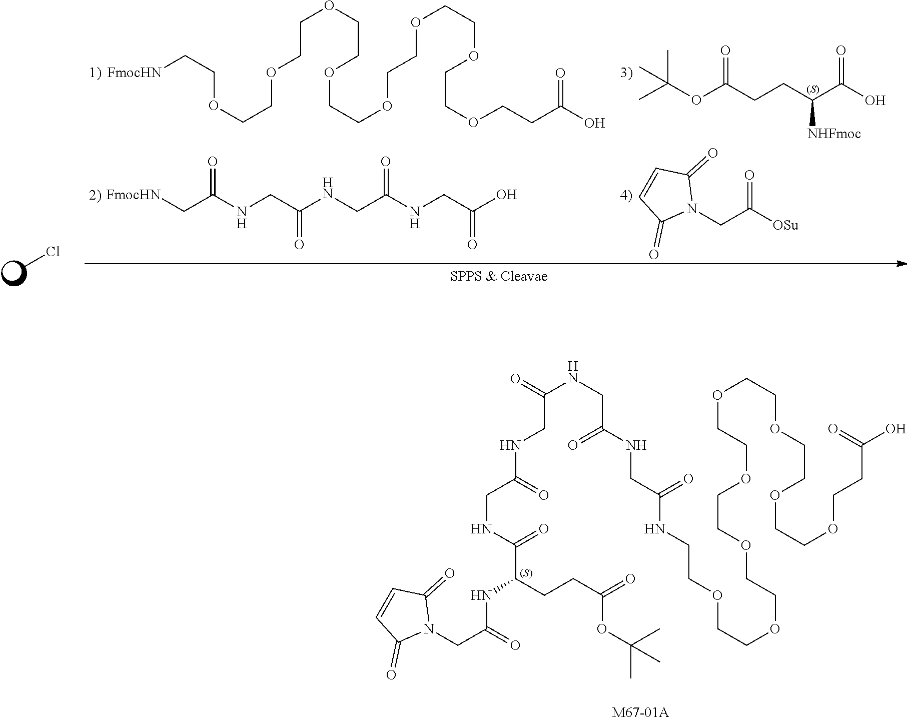

Trt-Cl (8.0 g, 8.8 mmol) and dichloromethane (160 mL) were added at room temperature. The Trt-Cl resin (8 g, 8.8 mmol) was swelling for 30 min. After filtration, the Trt-Cl resin was washed with N,N-dimethylformamide (3×100 mL). Then N,N-dimethylformamide (160 mL), 1-{[(9H-fluoren-9-ylmethoxy)carbonyl]amino}-3,6,9,12,15,18,21,24-octaoxaheptacosan-27-oic acid (11.74 g, 17.681 mmol, 0.69 equiv) and N,N-diisopropylethylamine (11.36 g, 87.890 mmol, 3.43 equiv) were added in sequence. Then the column was bubbled with nitrogen atmosphere for 2 h. After filtration, the residue was washed with N,N-dimethylformamide (3×100 mL), 150 mL dichloromethane/methanol/N,N-diisopropylethylamine (80/15/5) for 30 min, methanol (3×100 mL), dichloromethane (3×100 mL). The residue was dried by nitrogen atmosphere for 2h. To the above residue was added 20% piperdine (160 mL in N,N-dimethylformamide) at room temperature. The resulting mixture was stirred for additional lh at room temperature. After filtration, the residue was washed with N,N-dimethylformamide (3×100 mL). Then {2-[2-(2-{[(9H-fluoren-9-ylmethoxy)carbonyl]amino}acetamido)acetamido]acetamido}acetic acid (11.76 g, 25.095 mmol, 1.5 equiv), N,N-dimethylformamide (160 mL), N,N-diisopropylethylamine (17.30 g, 133.840 mmol, 8 equiv) and 2-(7-Azabenzotriazol-1-yl)-N,N,N′,N′-tetramethyluronium hexafluorophosphate (25.45 g, 66.920 mmol, 4 equiv) were added in sequence. Then the column was bubbled with nitrogen atmosphere for 2 h. After filtration, the residue was washed with N,N-dimethylformamide (3×100 mL). To the above residue 20% piperdine (160 mL in N,N-dimethylformamide) was added at room temperature. The resulting mixture was stirred for additional lh at room temperature. After filtration, the residue was washed with N,N-dimethylformamide (3×100 mL).

Then (2S)-5-(tert-butoxy)-2-{[(9H-fluoren-9-ylmethoxy)carbonyl]amino}-5-oxopentanoic acid (10.13 g, 23.797 mmol, 1.5 equiv), N,N-dimethylformamide (180 mL), N,N-diisopropylethylamine (16.40 g, 126.920 mmol, 8 equiv) and 2-(7-Azabenzotriazol-1-yl)-N,N,N′,N′-tetramethyluronium hexafluorophosphate (24.13 g, 63.460 mmol, 4 equiv) were added in sequence. Then the column was bubbled with nitrogen atmosphere for 2 h. After filtration, the residue was washed with N,N-dimethylformamide (3×100 mL). To the above residue 20% piperdine (160 mL in N,N-dimethylformamide) was added at room temperature. The resulting mixture was stirred for additional lh at room temperature. After filtration, the residue was washed with N,N-dimethylformamide (3×100 mL). Then 2,5-dioxopyrrolidin-1-yl 2-(2,5-dioxopyrrol-1-yl)acetate (4.00 g, 15.865 mmol, 1 equiv), N,N-dimethylformamide (180 mL), and triethylamine (8.03 g, 79.325 mmol, 5 equiv) was added in sequence. Then the column was bubbled with nitrogen atmosphere for 2 h. After filtration, the residue was washed with N,N-dimethylformamide (3×100 mL), dichloromethane (3×100 mL). Then 20 mL 20% 1,1,1,3,3,3-Hexafluoro-2-propanol in dichloromethane (180 ml) was added and bubbled with nitrogen atmosphere for 2 h. The resulting mixture was concentrated under reduced pressure. There was 1-{2-[2-(2-{2-[(2S)-5-(tert-butoxy)-2-[2-(2,5-dioxopyrrol-1-yl)acetamido]-5-oxopentanamido]acetamido}acetamido) acetamido]acetamido}-3,6,9,12,15,18,21,24-octaoxaheptacosan-27-oic acid (9.6 g, 26.6%) obtainedas a brown oil. LCMS (ES, m/z)[M+H]⁺=992.55. ¹H NMR (300 MHz, DMSO-d₆) δ 11.90 (s, 1H), 8.42 (d, J=7.9 Hz, 1H), 8.27 (t, J=5.7 Hz, 1H), 8.11 (dd, J=20.5, 5.9 Hz, 2H), 8.10 (s, 5H), 7.86 (t, J=5.6 Hz, 1H), 7.09 (d, J=1.4 Hz, 2H), 5.75 (d, J=1.3 Hz, 1H), 5.10-5.16 (m, 5H), 4.30-4.33 (m, 1H), 4.13-4.16 (m, 2H), 3.75 (m, 1OH), 3.60 (m, 3H), 3.41 (t, J=5.9 Hz, 6H), 3.37 (s, 3H), 3.22 (q, J=5.9 Hz, 2H), 2.43-2.46 (m, 3H), 2.23 (t, J=7.9 Hz, 2H), 1.90-1.94 (m, 1H), 1.72-1.76 (m, 1H), 1.40 (d, J=1.4 Hz, 12H).

### Step b:

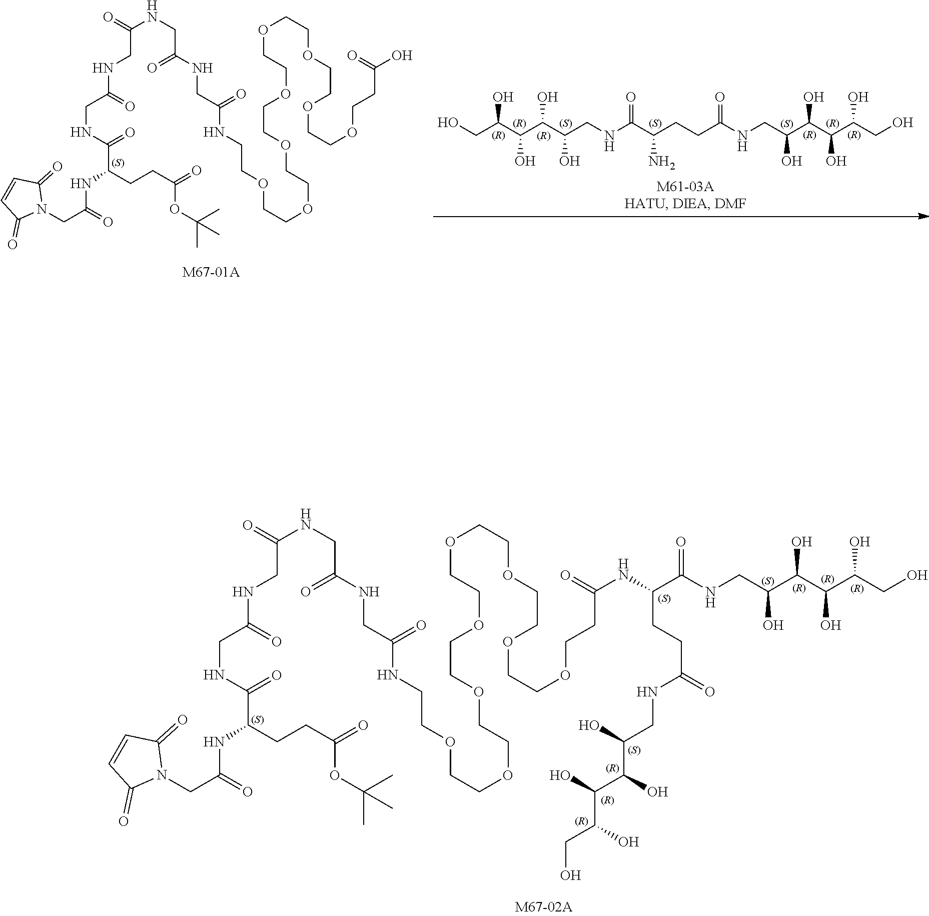

1-{2-[2-(2-{2-[(2S)-5-(tert-butoxy)-2-[2-(2,5-dioxopyrrol-1-yl)acetamido]-5-oxopentanamido]acetamido}acetamido)acetamido]acetamido}-3,6,9,12,15,18,21,24-octaoxaheptacosan-27-oic acid (1000 mg, 1.008 mmol, 1 equiv), (2S)-2-amino-N,N′-bis[(2S,3R,4R,5R)-2,3,4,5,6-pentahydroxyhexyl]pentanediamide (477.27 mg, 1.008 mmol, 1 equiv), N,N-diisopropylethylamine (260.57 mg, 2.016 mmol, 2 equiv), 2-(7-Azabenzotriazol-1-yl)-N,N,N′,N′-tetramethyluronium hexafluorophosphate (574.93 mg, 1.512 mmol, 1.5 equiv) and N,N-dimethylformamide (15 mL) were added at room temperature. The resulting mixture was stirred for 2 h at room temperature. The reaction was quenched by the addition of water (0.1% trifluoroacetic acid) (5 mL) at room temperature. The residue was purified by reverse flash chromatography with the following conditions: column, C18 gel; mobile phase, acetonitrile in water (0.1% trifluoroacetic acid), 10% to 50% gradient in 20 min; detector, UV 200 nm. There was tert-butyl (4S)-4-({[({[({[(26-{[(1S)-1,3-bis({[(2S,3R,4R,5R)-2,3,4,5,6-pentahydroxyhexyl]carbamoyl)propyl]carbamoyl}-3,6,9,12,15,18,21,24-octaoxahexacosan-1-yl)carbamoyl]methyl}carbamoyl)methyl]carbamoyl}methyl)carbamoyl]methyl}carbamoyl)-4-[2-(2,5-dioxopyrrol-1-yl) acetamido]butanoate (710 mg, 48.66%) obtained as a brown oil. LCMS (ES, m/z): [M+2H]²⁺=724.70. ¹H NMR (300 MHz, DMSO-d₆) δ 8.41 (d, J=8.0 Hz, 1H), 8.25 (d, J=5.9 Hz, 1H), 8.0-8.12 (m, 6.8 Hz, 5H), 7.86 (t, J=5.6 Hz, 1H), 7.70-7.74 (m, 2H), 7.09 (s, 2H), 4.28-4.32 (m, 1H), 4.22 (s, 2H), 4.11 (d, J=4.2 Hz, 2H), 3.75 (d, J=5.3 Hz, 7H), 3.68 (d, J=5.6 Hz, 2H), 3.61 (q, J=5.3, 3.2 Hz, 8H), 3.59 (q, J=5.3, 3.2 Hz, 7H), 3.51 (s, 30H), 3.49 (s, 3H), 3.40 (d, J=6.7 Hz, 8H), 3.24 (dd, J=14.0, 8.3 Hz, 4H), 3.14 (s, 1H), 3.00-3.04 (m, 2H), 2.50 (d, J=5.8 Hz, 3H), 2.39 (s, 2H), 2.22 (t, J=7.7 Hz, 2H), 1.89 (m, 2H), 1.70-1.73 (m, 2H), 1.39 (s, 9H), 1.39 (s, 9H), 1.12 (m, 1H).

### Step c:

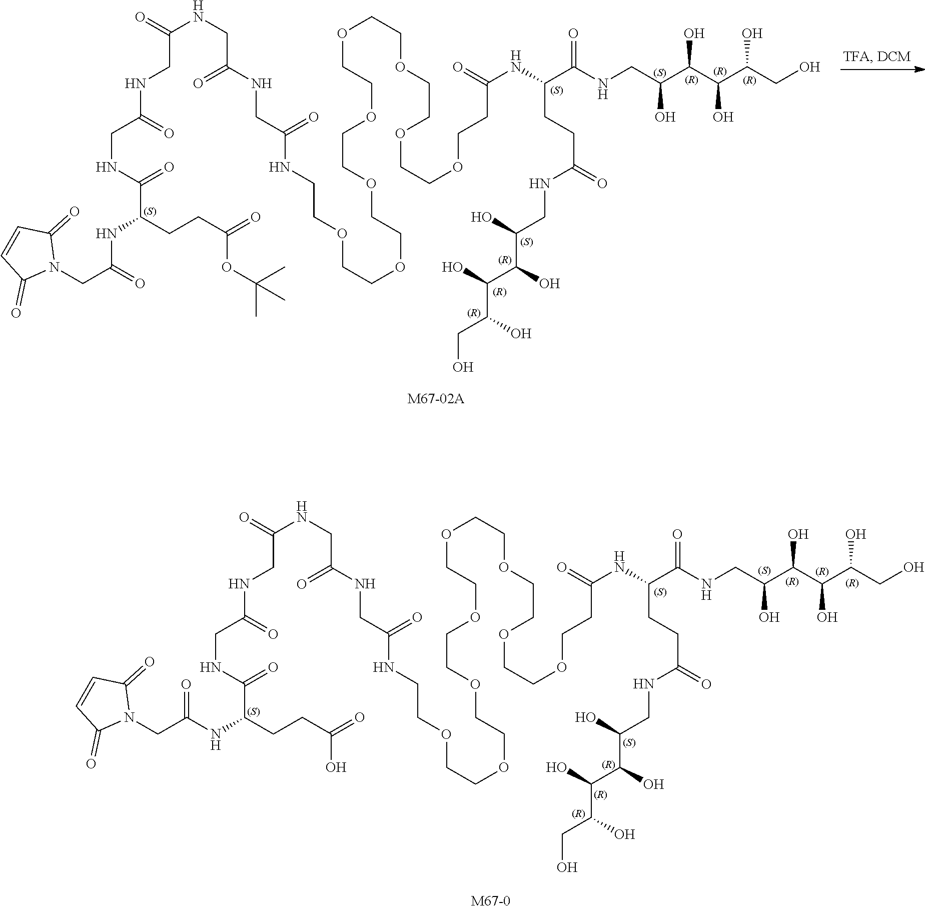

Tert-butyl (4S)-4-({[({[({[(26-{[(1S)-1,3-bis({[(2S,3R,4R,5R)-2,3,4,5,6-pentahydroxyhexyl]carbamoyl}) propyl]carbamoyl}-3,6,9,12,15,18,21,24-octaoxahexacosan-1-yl)carbamoyl]methyl}carbamoyl)methyl]carbamoyl}methyl)carbamoyl]methyl}carbamoyl)-4-[2-(2,5-dioxopyrrol-1-yl)acetamido]butanoate (400 mg, 0.276 mmol, 1 equiv), dichloromethane (8 mL) and trifluoroacetic acid (8 mL) were added at room temperature. The resulting mixture was stirred for 2 h at room temperature. The crude product (270 mg) was purified by Prep-HPLC with the following conditions (Column: XBridge Prep OBD C18 Column, 30*150 mm, ₘ; Mobile Phase A: water(0.05% TFA ), Mobile Phase B: acetonitrile; Flow rate: 60 mL/min; Gradient: 3% B to 15% B in 9 min, 15% B; Wave Length: 220 nm; RT1(min): 8; Number Of Runs: 0) to obtain (4S)-4-({[({[({[(26-{[(1S)-1,3-bis({[(2S,3R,4R,5R)-2,3,4,5,6-pentahydroxyhexyl]carbamoyl})propyl]carbamoyl}-3,6,9,12,15,18,21,24-octaoxahexacosan-1-yl)carbamoyl]methyl}carbamoyl)methyl]carbamoyl}methyl) carbamoyl]methyl}carbamoyl)-4-[2-(2,5-dioxopyrrol-1-yl)acetamido]butanoic acid (170.0 mg, 43.49%) as a colorless semi-solid. LCMS (ES, m/z): [M+H]⁺=1391.7. ¹H NMR (300 MHz, Deuterium Oxide) 6 6.86 (s, 2H), 4.73 (s, 2H), 4.23-4.26 (m, 4H), 3.88-3.92 (m, 6H), 3.84 (s, 2H), 3.72-3.76 (m, 10H), 3.57-3.60 (m, 32H), 3.34-3.38 (m, 4H), 3.18-3.22 (m, 2H), 2.52 (td, J=5.9, 2.8 Hz, 2H), 2.43 (t, J=7.3 Hz, 2H), 2.29 (t, J=7.6 Hz, 2H), 1.92 (tt, J=14.8, 7.5 Hz, 4H).

### Example III-4 Synthesis of Linker Compound M72-0

### Step a:

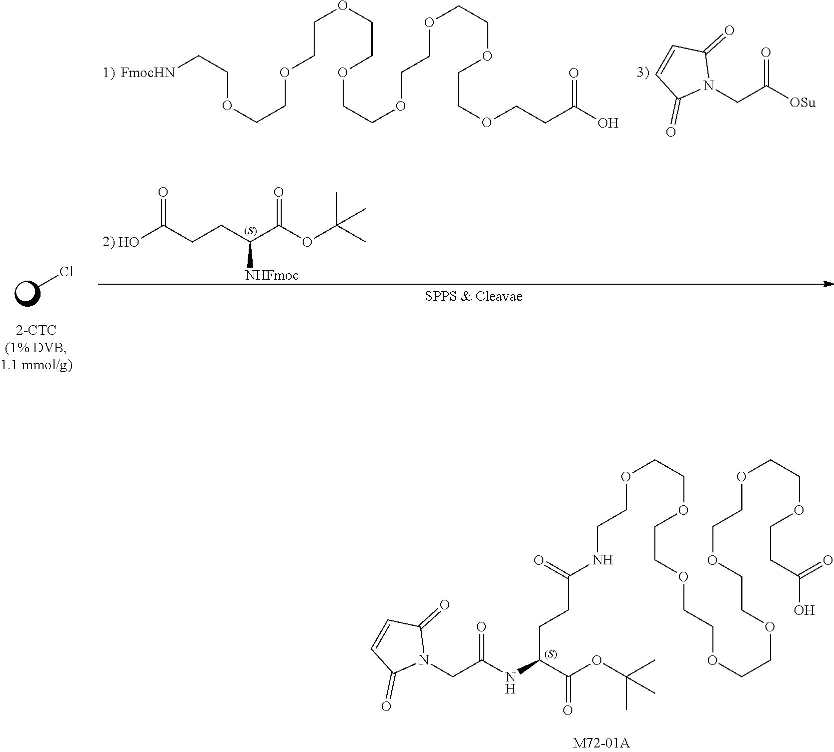

Trt-resin (2.0 g, 2.788 mmol, 1 equiv) and dichloromethane (45 mL) were added at room temperature. The Trt-Cl resin was swelling for 30 min. After filtration, the Trt-Cl resin was washed with N, N-dimethylformamide (3×20 mL). Then N, N-dimethylformamide (60 mL) and 1-(9H-fluoren-9-yl)-3-oxo-2-7,10,13,16,19,22,25,28-nonaoxa-4-azahentriacontan-31-oic acid (4.53 g, 6.822 mmol, 0.71 equiv) were added in sequence. Then the column was bubbled with nitrogen atmosphere for 2 h. After filtration, the residue was washed with N, N-dimethylformamide (3×45 mL), 60 mL dichloromethane/methanol/N, N-diisopropylethylamine (80%/15%/5%) for 20 min, then dichloromethane (3×45 mL). The residue was dried by nitrogen atmosphere for 2 h. To the above residue 20 mL 20% piperdine in N, N-dimethylformamide was added at room temperature. The resulting mixture was stirred for additional 1 h at room temperature. After filtration, the residue was washed with N, N-dimethylformamide (3×45 mL) and dichloromethane (3×45 mL). The residue was dried by nitrogen atmosphere for 2 h. There was 4-{[(1-amino-3,6,9,12,15,18,21,24-octaoxaheptacosanoyl)oxy]-Trt resin (2.4 g, 63.84%) obtained as a yellow powder. The product was used directly in the next step. 4-{[(1-amino-3,6,9,12,15,18,21,24-octaoxaheptacosanoyl) oxy]-Trt resin (2.4 g, 3.34 mmol, 1 equiv) and dichloromethane (60 mL) were added at room temperature. The Trt-Cl resin was swelling for 30 min. After filtration, the Trt-Cl resin was washed with N, N-dimethylformamide (3×40 mL). To the above residue (S)-4-((((9H-fluoren-9-yl)methoxy)carbonyl)amino)-5-(tert-butoxy)-5-oxopentanoic acid (7.1 g, 16.7 mmol, 5.0 equiv), N, N-diisopropylethylamine (4.3 g, 33.3 mmol, 10 equiv) and 2-(7-Azabenzotriazol-1-yl)-N, N, N′, N′-tetramethyluronium hexafluorophosphate (6.4 g, 16.7 mmol, 5 equiv) was added at room temperature. The resulting mixture was stirred for additional 1 h at room temperature. After filtration, the residue was washed with N, N-dimethylformamide (3×15 mL) and dichloromethane (3×15 mL). Then 20 mL 20% 1,1,1,3,3,3-Hexafluoro-2-propanol in N, N-dimethylformamide was added. The mixture was bubbled by N₂ for 2 h. After filtration, the filtrate was concentrated under reduced pressure. There was [4-({[(4S)-5-(tert-butoxy)-4-[2-(2,5-dioxopyrrol-1-yl)acetamido]-5-oxopentanoyl]oxy}(2-chlorophenyl)pheny--lmethyl)phenyl]methyl group obtained as a colorless oil. To the above residue 50 mL 20% piperdine in N, N-dimethylformamide was added at room temperature. The resulting mixture was stirred for additional lh at room temperature. After filtration, the residue was washed with N, N-dimethylformamide (3×15 mL) and dichloromethane (3×15 mL). There was {4-[({1-[(4S)-4-amino-5-(tert-butoxy)-5-oxopentanamedo]-3,6,9,12,15,18- -21,24-octaoxaheptacosanoyl}oxy)(2-chlorophenyl)phenylmethyl]phenyl}methyl group obtained as a yellow powder. The residue was dried by nitrogen atmosphere for 2 h and used in the next step directly. {4-[({1-[(4S)-4-amino-5-(tert-butoxy)-5-oxopentanamed- -o]-3,6,9,12,15,18,21,24-octaoxaheptacosanoyl}oxy)(2-chlorophenyl)phenylmethyl]phenyl}methyl group (5.8 g, 1.6 mmol, 1 equiv) and dichloromethane (20 mL) were added at room temperature. The resin was swelling for 30 min. After filtration, the resin was washed with N, N-dimethylformamide (3×20 mL). Then N-Succinimidyl maleimidoacetate (0.47 g, 1.9 mmol, 1.2 equiv), N, N-dimethylformamide (30 mL), and trimethylamine (0.63 g, 6.4 mmol, 4 equiv) were added in sequence. Then the column was bubbled with nitrogen atmosphere for 2 h.

After filtration, the residue was washed with N, N-dimethylformamide (3×20 mL), dichloromethane (3×20 mL). The residue was dried by nitrogen atmosphere for 2 h. Then 20 mL 20% hexafluoroisopropanol in dichloromethane (20 ml) was added and bubbled with nitrogen atmosphere for 2 h. There was 1-[(4S)-5-(tert-butoxy)-4-[2-(2,5-dioxopyrrol-1-yl)- -acetamido]-5-oxopentanamido]-3,6,9,12,15,18,21,24-octaoxaheptacosan-27-oic acid (1.2 g, 13.77%) obtained as a colorless oil. LCMS: [M+H]⁺=764.50. ¹H NMR (300 MHz, DMSO-d₆) δ 12.02 (s, 1H), 8.49 (s, 1H), 8.04 (s, 1H), 7.90 (s, 1H), 7.10 (d, J=3.2 Hz, 2H), 5.75 (s, 1H), 5.15 (s, 1H), 4.06 (s, 3H), 3.59 (q, J=5.9 Hz, 3H), 3.50 (t, J=3.0 Hz, 22H), 3.39 (d, J=4.7 Hz, 3H), 3.19 (d, J=6.6 Hz, 2H), 2.93-2.68 (m, 1H), 2.43 (d, J=5.3 Hz, 2H), 2.13 (s, 2H), 1.83 (d, J=37.2 Hz, 2H), 1.39 (t, J=3.1 Hz, 9H).

### Step b:

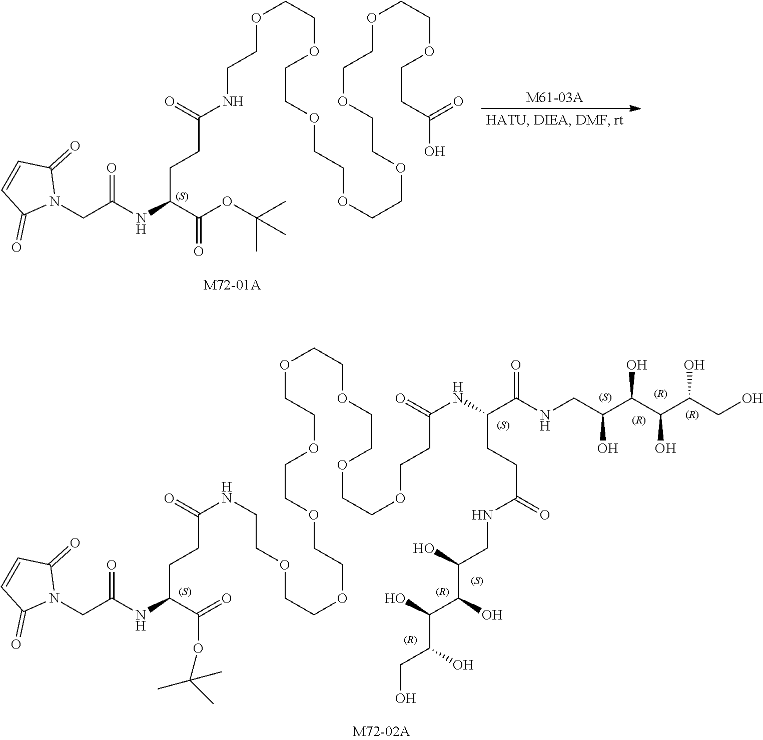

1-[(4S)-5-(tert-butoxy)-4-[2-(2,5-dioxopyrrol-1-yl)acetamido]-5- -oxopentanamido]-3,6,9,12,15,18,21,24-octaoxaheptacosan-27-oic acid (1 g, 1.309 mmol, 1 equiv), (2S)-2-amino-N,N′-bis[(2S,3R,4R,5R)-2,3,4,5,6-pentahydroxyhexyl]pentanediamide (0.62 g, 1.309 mmol, 1.00 equiv), N,N-diisopropylethylamine (0.34 g, 2.618 mmol, 2.00 equiv), 2-(7-Azabenzotriazol-1-yl)-N, - —N,N′,N′-tetramethyluronium hexafluorophosphate (1.00 g, 2.618 mmol, 2.00 equiv) and N, N-dimethylformamide (10 mL) were added at room temperature. The resulting mixture was stirred for 2 h at room temperature. The residue was purified by reverse flash chromatography with the following conditions: column, C18 gel; mobile phase, acetonitrile in water, 10% to 50% gradient in 20 min; detector, UV 200 nm. There was tert-butyl (2S)-4-[(26-{[(1S)-1,3-bis({[(2S,3R,4R,5R)-2,3,4,5,6-pentahydroxyhexyl]carbamoyl})propyl]carbamoyl}-3,6,9,12,15,18,21,24-octaoxahexacosan-1-yl)carbamoyl]-2-[2-(2,5-dioxopyrrol-1-yl)acetamido]butanoate (600 mg, 37.59%) obtained as a brown oil. LCMS (ES, m/z): [M+H]⁺=1219.75. ¹H NMR (300 MHz, DMSO-d₆) δ 8.51 (d, J=6.7 Hz, 1H), 8.06-7.85 (m, 2H), 7.71 (d, J=14.4 Hz, 2H), 7.09 (d, J=3.8 Hz, 2H), 4.92 (s, 38H), 4.22 (s, 2H), 4.06 (s, 3H), 3.63-3.52 (m, 9H), 3.38 (s, 5H), 3.27 (s, 2H), 3.23-3.15 (m, 3H), 3.03 (d, J=7.8 Hz, 2H), 2.39 (s, 2H), 2.17-2.07 (m, 4H), 1.80 (d, J=43.2 Hz, 5H), 1.38 (d, J=3.8 Hz, 9H).

### Step c:

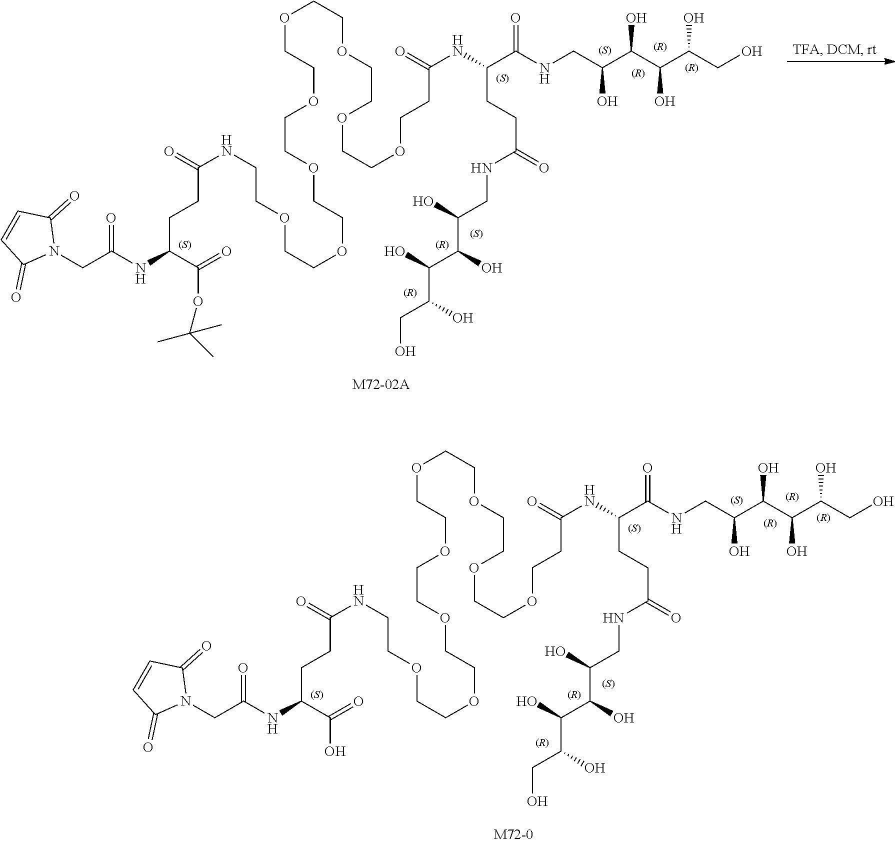

Tert-butyl (2S)-4-[(26-{[(1S)-1,3-bis({[(2S,3R,4R,5R)-2,3,4,5,6- -pentahydroxyhexyl]carbamoyl})propyl]carbamoyl}-3,6,9,12,15,18,21,24-octaoxahexacosan-1-yl)carbamoyl]-2-[2-(2,5-dioxopyrrol-1-yl)acetamido]butanoate (600 mg, 0.492 mmol, 1 equiv) and dichloromethane (3 mL) were added at room temperature. To the above mixture trifluoroacetic acid (3 mL) was added dropwise at room temperature. The resulting mixture was stirred for additional 40 min at room temperature. Desired product could be detected by LCMS. The resulting mixture was concentrated under reduced pressure. The reaction was quenched by the addition of water (5 mL) at room temperature. The residue was purified by reverse flash chromatography with the following conditions: column, C18; mobile phase, acetonitrile in water (0.1% trifluoroacetic acid), 10% to 50% gradient in 30 min; detector, UV 254 nm. The crude product [300 mg(80%)] was purified by Prep-HPLC with the following conditions (Column: XBridge Prep OBD C18 Column, 30*150 mm, 5 μm ; Mobile Phase A: water(0.05% trifluoroacetic acid ), Mobile Phase B: acetonitrile; Flow rate: 60 mL/min; Gradient: 3% B to 15% B in 9 min, 15% B; Wave Length: 254 nm; RT1(min): 7; Number Of Runs: 0) to obtain (2S)-4-[(26-{[(1S)-1,3-bis({[(2S,3R,4R,5R)-2,3,4,5,6-pentahydroxyhexyl]carbamoyl})propyl]carbamoyl}-3,6,9,12,15,18,21,24-octaoxahexacosan-1-yl)carbamoyl]-2-[2-(2,5-dioxopyrrol-1-yl)acetamido]butanoic acid (163.3 mg, 28.53%) as a white solid. LCMS (ES, m/z): [M+H]⁺=1163.40. ¹H NMR (300 MHz, Deuterium Oxide) 6 6.96 (d, J=3.1 Hz, 2H), 4.45-4.27 (m, 4H), 3.81 (m, 11H), 3.70 (m, 33H), 3.53-3.35 (m, 4H), 3.28 (s, 2H), 2.61 (m, 2H), 2.37 (d, J=8.0 Hz, 4H), 2.30-1.91 (m, 4H).

### Example III-5 Synthesis of Linker Compound 373

### Step a: General procedure for preparation of Compound 3

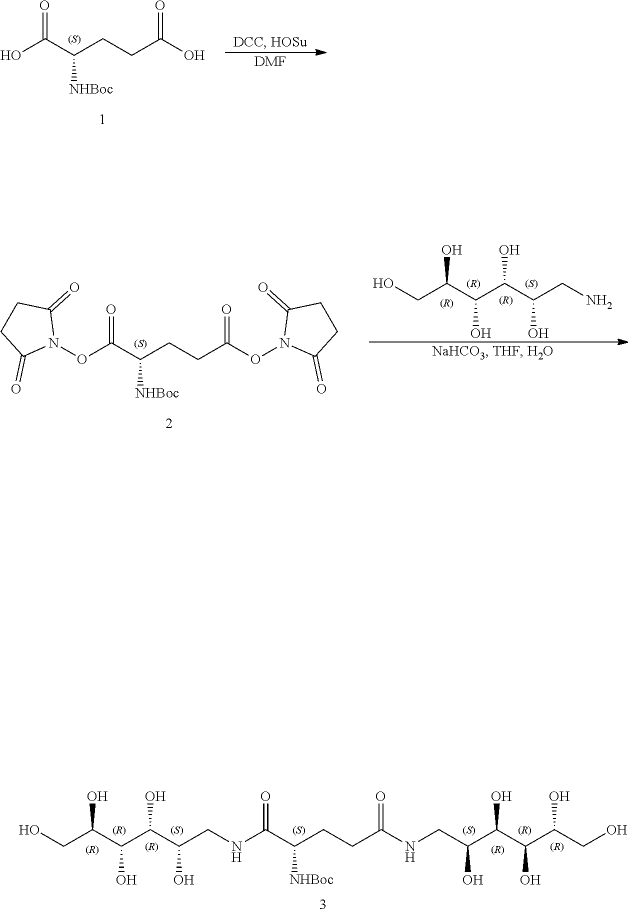

A mixture of (tert-butoxycarbonyl)-L-glutamic acid (11.37 g, 45.99 mmol, 1.00 equiv.), DCC (18.98 g, 91.99 mmol, 18.61 mL, 2.00 equiv.), DMAP (1.12 g, 9.20 mmol, 0.20 equiv.) and HOSu (21.17 g, 183.97 mmol, 4.00 equiv.) in THF (400 mL) was stirred at 0° C. for 0.5 hr. Then the mixture was filtered to remove undissolved residue. The filterate was added to a solution of (2R,3R,4R,5S)-6-aminohexane-1,2,3,4,5-pentol (25.00 g, 137.98 mmol, 3.00 equiv.) and NaHCO₃ (7.73 g, 91.99 mmol, 3.58 mL, 2.00 equiv.) in H₂O (200 mL), then the reaction mixture was stirred at 20° C. for 2 hrs. LCMS showed (tert-butoxycarbonyl)-L-glutamic acid was consumed completely and one main peak with desired m/z was detected. The reaction mixture was concentrated to remove THF and purified by prep-HPLC (TFA condition) to obtain tert-butyl ((S)-1,5-dioxo-1,5-bis(((2S,3R,4R,5R)-2,3,4,5,6-pentahydroxyhexyl)amino)pentan-2-yl)carbamate (13.00 g, 22.66 mmol, 97.1% purity, 47.85% yield) as a white solid.

### Step b: General Procedure for Preparation of Compound 4

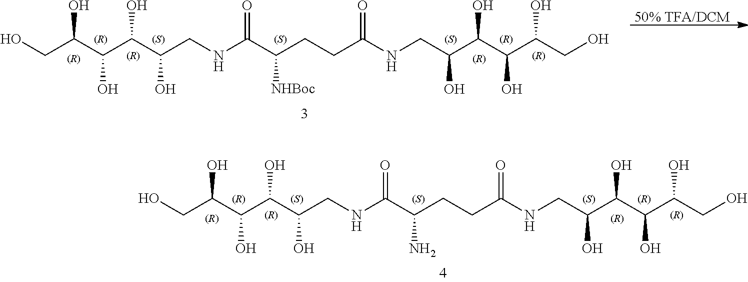

Tert-butyl ((S)-1,5-dioxo-1,5-bis(((2S,3R,4R,5R)-2,3,4,5,6-pentahydroxyhexyl)amino)pentan-2-yl)carbamate (13.00 g, 22.66 mmol, 97.1% purity) was added to a solution of TFA (100 mL) in DCM (100 mL) in one portion at 25° C. . The mixture was stirred at 25° C. for 30 min. LCMS showed tert-butyl ((S)-1,5-dioxo-1,5-bis(((2S,3R,4R,5R)-2,3,4,5,6-pentahydroxyhexyl)amino)pentan-2-yl)carbamate was consumed completely and one main peak with desired m/z or desired mass was detected. The solvent was removed under reduced pressure to give the (S)-2-amino-N₁,N₅-bis((2S,3R,4R,5R)-2,3,4,5,6-pentahydroxyhexyl)pentanediamide (11.99 g, crude).

### Step c: General Procedure for Preparation of Compound 5

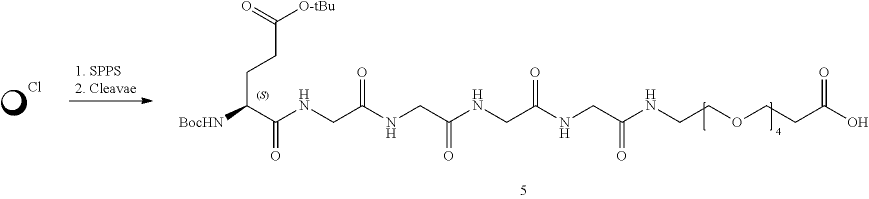

The peptide was synthesized using standard Fmoc chemistry.

DCM was added to the vessel containing CTC Resin (30.00 mmol, 55.30 g, 0.65 mmol/g) and Fmoc-PEG4-CH₂CH₂COOH (14.16 g, 30.00 mmol, 1.00 equiv.) with N₂ bubbling.

DIEA (4.0 equiv.) was added dropwise and mixed for 2 hrs.

MeOH (30.0 mL) was added and mixed for 30 mins.

The reaction mixture was drained and washed with DMF for 5 times. 20% piperidine/DMF was added and mixed for 30 mins.

The resultant was drained and then washed with DMF for30 seconds for 5 times.

Fmoc-amino acid solution was added and mixed for 30 seconds, and then activation buffer was added, and bubbled with N₂ for about 1 hr. 20% piperidine/DMF was added and mixed for 30 min.

Steps 4 to 8 were repeated for the subsequent amino acid coupling step.

Note: Synthesis scale: 30.00 mmol.

The coupling reaction was monitored by ninhydrin test, and the resin was washed with DMF for 5 times.

The resin was washed with MeOH for 3 times and dried by vacuum.

### Step d: Peptide Cleavage

Cleavage buffer (20% HFIP/DCM) was added to the flask containing the side chain protected peptide at room temperature and stirred for 30 min twice.

The reaction mixture was filteredand the solvent was removed under vacuum.

(S)-6-(3-(tert-butoxy)-3-oxopropyl)-2,2-dimethyl-4,7,10,13,16,19-hexaoxo-3,23,26,29,32-pentaoxa-5,8,11,14,17,20-hexaazapentatriacontan-35-oic acid (19.10 g, crude) was obtained as a white solid.

### Step e: General Procedure for Preparation of Compound 6

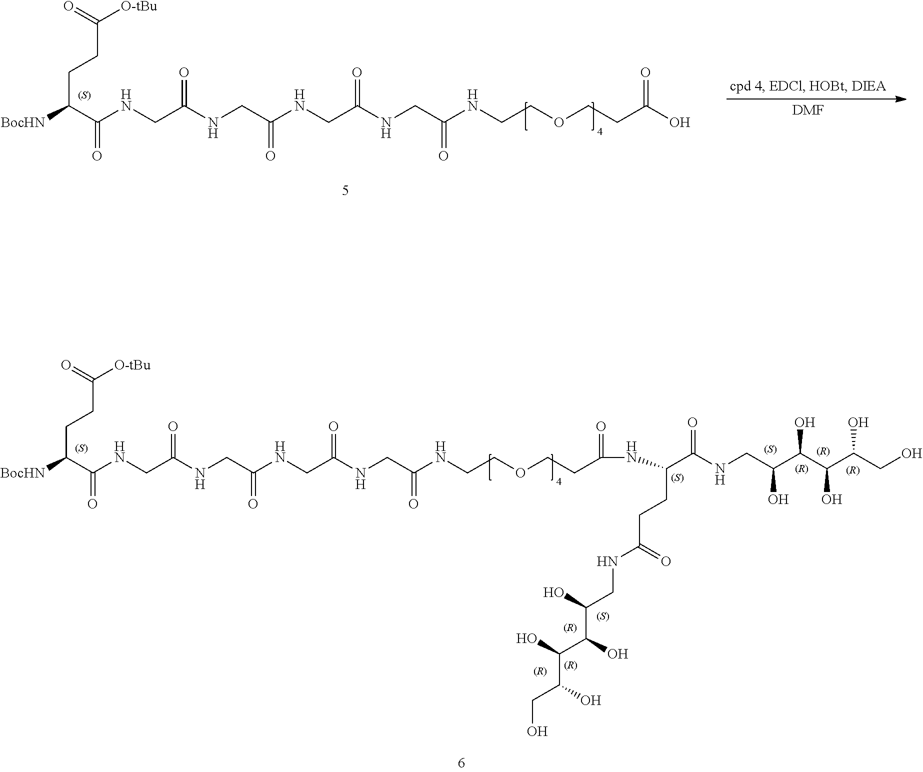

To a mixture of (S)-6-(3-(tert-butoxy)-3-oxopropyl)-2,2-dimethyl-4,7,10,13,16,19-hexaoxo-3,23,26,29,32-pentaoxa-5,8,11,14,17,20-hexaazapentatriacontan-35-oic acid (6.08 g, 9.42 mmol, 1.00 equiv.) and (S)-2-amino-N₁,N₅-bis((2S,3R,4R,5R)-2,3,4,5,6-pentahydroxyhexyl)pentanediamide (6.69 g, 14.14 mmol, 1.50 equiv.) in DMF (100 mL) EDCI (3.61 g, 18.85 mmol, 2.00 equiv.), HOBt (2.55 g, 18.85 mmol, 2.00 equiv.) and DIEA (3.65 g, 28.27 mmol, 4.92 mL, 3.00 equiv.) were added in one portion at 25° C. The mixture was stirred for 12 hrs. LCMS showed that the reaction was completed. The mixture was acidified to pH=7 by 1 M HCl. Then the mixture was purified by prep-HPLC (A: 0.075% TFA in H₂O, B: ACN) to afford tert-butyl (4S,35S,41S,42R,43R,44R)-4-((tert-butoxycarbonyl)amino)-41,42,43,44,45-pentahydroxy-5,8,11,14,17,33,38-heptaoxo-35-(((2S,3R,4R,5R)-2,3,4,5,6-pentahydroxyhexyl)carbamoyl)-21,24,27,30-tetraoxa-6,9,12,15,18,34,39-heptaazapentatetracontanoate (7.5 g, 98.0% purity) as a colorless solid.

### Step f: General Procedure for Preparation of Compound 7

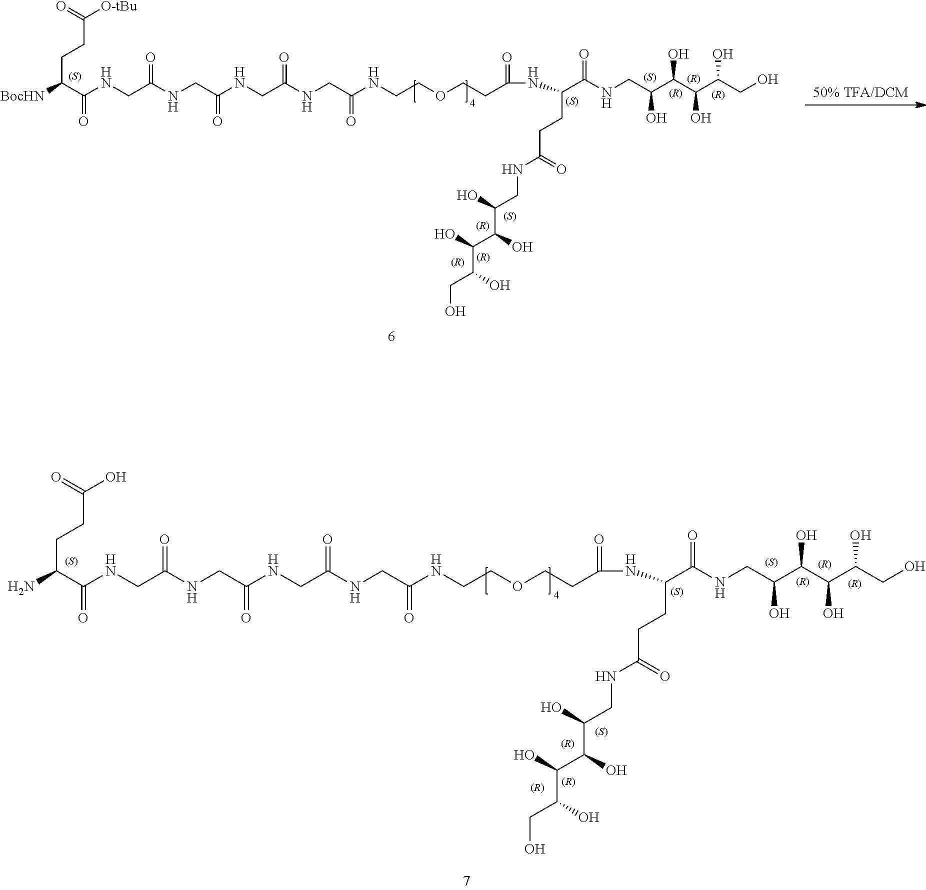

To a mixture of tert-butyl (4S,35S,41S,42R,43R,44R)-4-((tert-butoxycarbonyl)amino)-41,42,43,44,45-pentahydroxy-5,8,11,14,17,33,38-heptaoxo-35-(((2S,3R,4R,5R)-2,3,4,5,6-pentahydroxyhexyl)carbamoyl)-21,24,27,30-tetraoxa-6,9,12,15,18,34,39-heptaazapentatetracontanoate (7.5 g, 7.44 mmol, 1.00 equiv.) in DCM (30 mL), TFA (30 mL) was added in one portion at 25° C. The mixture was stirred for 30 mins. LCMS showed that the reaction was completed. The solvent was removed under reduced pressure to afford (4S,35S,41S,42R,43R,44R)-4-amino-41,42,43,44,45-pentahydroxy-5,8,11,14,17,33,38-heptaoxo-35-(((2S,3R,4R,5R)-2,3,4,5,6-pentahydroxyhexyl)carbamoyl)-21,24,27,30-tetraoxa-6,9,12,15,18,34,39-heptaazapentatetracontanoic acid (4.66 g, crude) as a colorless solid.

### Step g: General Procedure for Preparation of 373

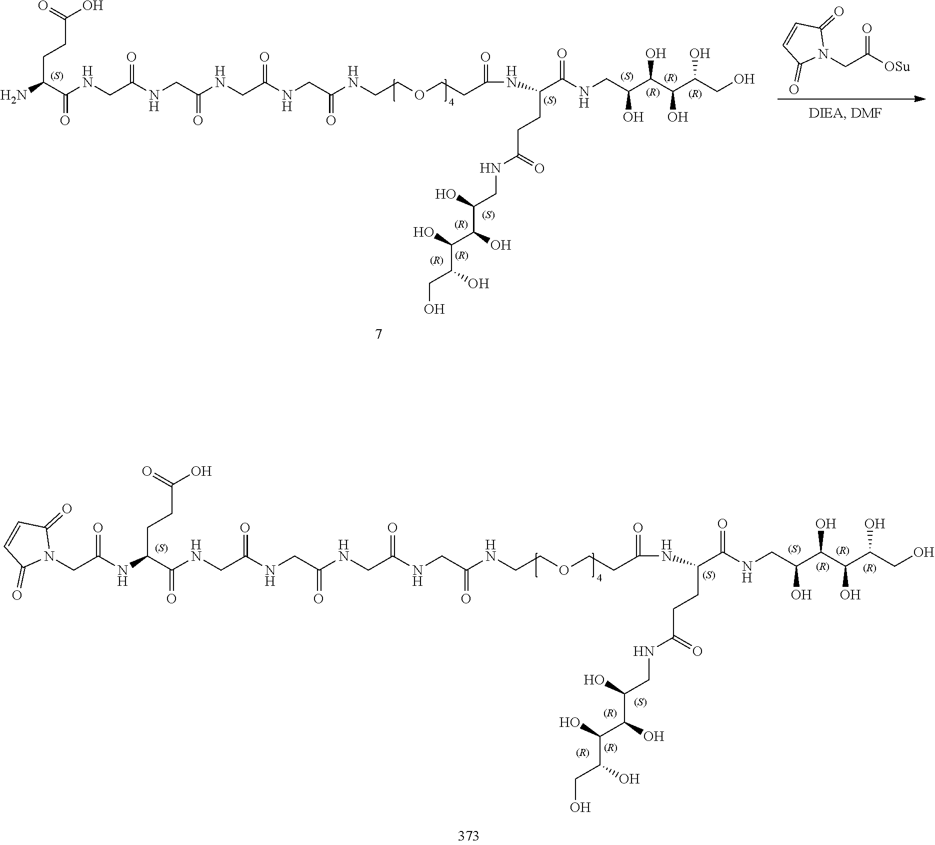

To a mixture of (4S,35S,41S,42R,43R,44R)-4-amino-41,42,43,44,45-pentahydroxy-5,8,11,14,17,33,38-heptaoxo-35-(((2S,3R,4R,5R)-2,3,4,5,6-pentahydroxyhexyl)carbamoyl)-21,24,27,30-tetraoxa-6,9,12,15,18,34,39-heptaazapentatetracontanoic acid (3.20 g, 2.55 mmol, 1.00 equiv.) and (2,5-dioxopyrrolidin-1-yl) 2-(2,5-dioxopyrrol-1-yl)acetate (965.06 mg, 3.83 mmol, 1.50 equiv.) in DMF (15 mL), DIEA (989.19 mg, 7.65 mmol, 1.33 mL, 3.00 equiv.) was added in one portion at 0° C. and reacted for 1 hr. LCMS was used to determine completion of the reaction. The crude peptide was purified by Prep-HPLC (A: 0.075% TFA in H₂O, B: ACN) to afford (4S,35S,41S,42R,43R,44R)-4-(2-(2,5-dioxo-2,5-dihydro-1H-pyrrol-1-yl)acetamido)-41,42,43,44,45-pentahydroxy-5,8,11,14,17,33,38-heptaoxo-35-(((2S,3R,4R,5R)-2,3,4,5,6-pentahydroxyhexyl)carbamoyl)-21,24,27,30-tetraoxa-6,9,12,15,18,34,39-heptaazapentatetracontanoic acid (batch 1, 723.0 mg, 95.1% purity; batch 2, 229.7 mg, 95.8% purity; batch3, 52.2 mg, 95% purity, 26.92% total yield) as a colorless solid.

### Example III-6 Synthesis of Linker Compound 374

### Step a: General Procedure for Preparation of Compound 8

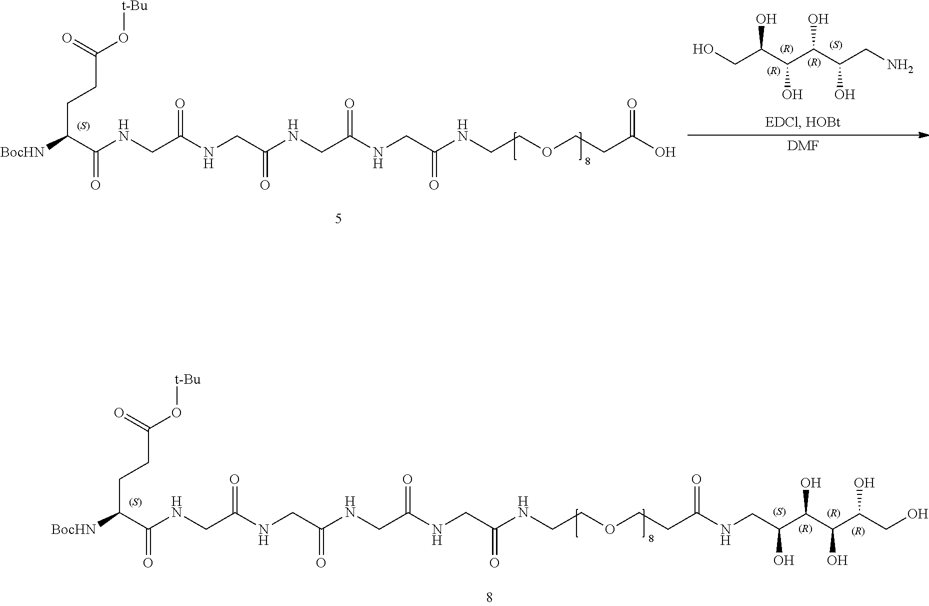

To a mixture of (S)-6-(3-(tert-butoxy)-3-oxopropyl)-2,2-dimethyl-4,7,10,13,16,19-hexaoxo-3,23,26,29,32,35,38,41,44-nonaoxa-5,8,11,14,17,20-hexaazaheptatetracontan-47-oic acid (1.00 g, 1.05 mmol, 1.00 equiv.) and (2R,3R,4R,5S)-6-aminohexane-1,2,3,4,5-pentol (284.57 mg, 1.57 mmol, 1.50 equiv.) in DMF (10 mL), EDCI (401.45 mg, 2.09 mmol, 2.00 equiv.) and HOBt (282.96 mg, 2.09 mmol, 2.00 equiv.) were added in one portion at 25° C. and reacted for 12 hrs. LCMS was used to determine completion of the reaction.

The solvent was removed under reduced pressure to afford tert-butyl (4S,48S,49R,50R,51R)-4-((tert-butoxycarbonyl)amino)-48,49,50,51,52-pentahydroxy-5,8,11,14,17,45-hexaoxo-21,24,27,30,33,36,39,42-octaoxa-6,9,12,15,18,46-hexaazadopentacontanoate (0.80 g, crude) as a colorless solid. General procedure for preparation of-Compound 9

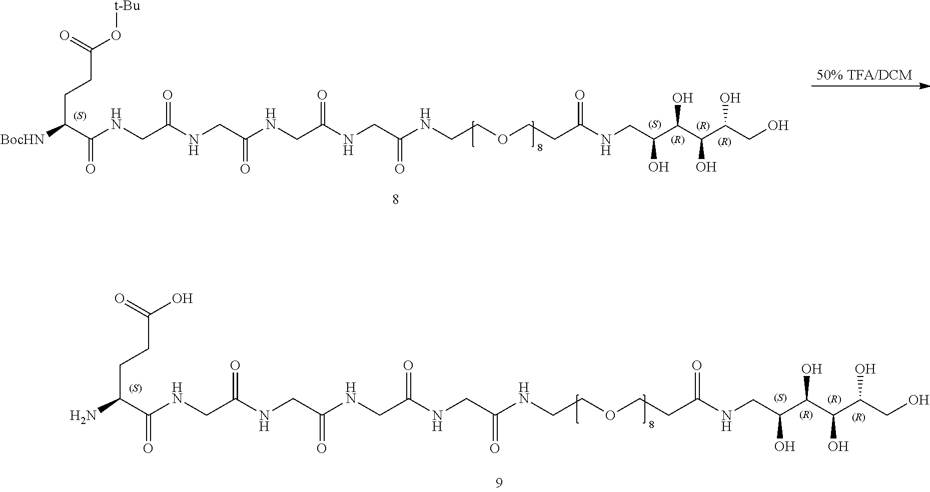

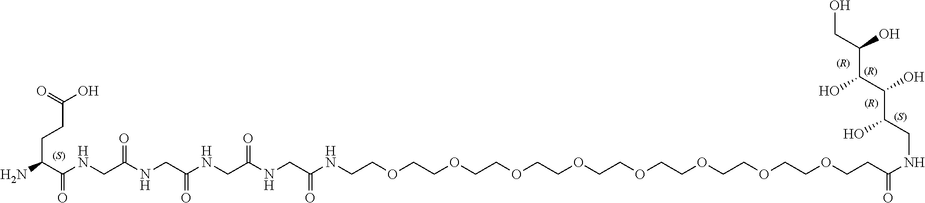

To a mixture of compound 8 (0.80 g, 715.42 umol, 1.00 equiv.) in DCM (5 mL) TFA (5 mL) was added in one portion at 25° C. and the mixture was stirred for 30 mins. LCMS was used to determine completion of the reaction. The solvent was removed under reduced pressure to afford (4S,48S,49R,50R,51R)-4-amino-48,49,50,51,52-pentahydroxy-5,8,11,14,17,45-hexaoxo-21,24,27,30,33,36,39,42-octaoxa-6,9,12,15,18,46-hexaazadopentacontanoic acid (0.50 g, crude) as a colorless.

### Step c: General procedure for preparation of 374

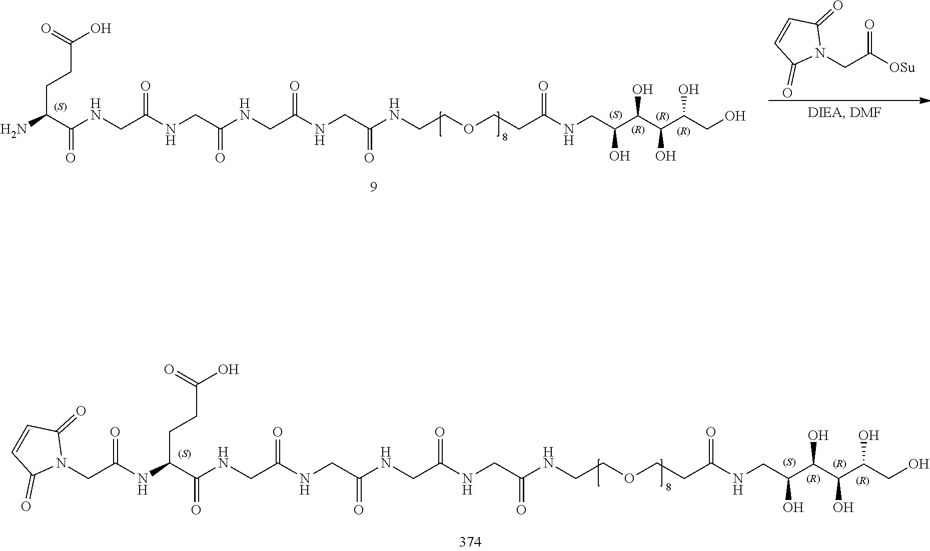

To a mixture of (4S,48S,49R,50R,51R)-4-amino-48,49,50,51,52-pentahydroxy-5,8,11,14,17,45-hexaoxo-21,24,27,30,33,36,39,42-octaoxa-6,9,12,15,18,46-hexaazadopentacontanoic acid (0.5 g, 519.75 umol, 1.00 equiv.) and (2,5-dioxopyrrolidin-1-yl) 2-(2,5-dioxopyrrol-1-yl)acetate (196.61 mg, 779.62 umol, 1.50 equiv.) in DMF (5 mL) DIEA (201.52 mg, 1.56 mmol, 271.59 μL, 3.00 equiv.) was added in one portion at 0° C. and reacted for 1 hr. LCMS showed that the reaction was completed. The mixture was purified by prep-HPLC (A: 0.075% TFA in H₂O, B: ACN) to afford (4S,48S,49R,50R,51R)-4-(2-(2,5-dioxo-2,5-dihydro-1H-pyrrol-1-yl)acetamido)-48,49,50,51,52-pentahydroxy-5,8,11,14,17,45-hexaoxo-21,24,27,30,33,36,39,42-octaoxa-6,9,12,15,18,46-hexaazadopentacontanoic acid (251.9 mg, 97.9% purity, 43.17% yield) as a colorless solid.

### Example III-7 Synthesis of linker compound 372

### Step a: General procedure for preparation of Compound 10

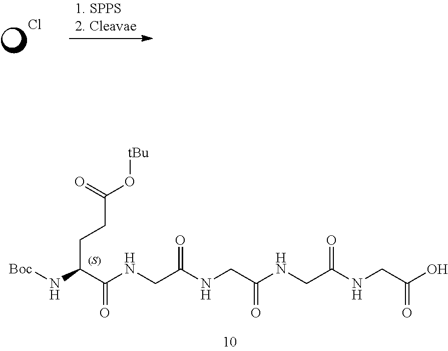

The peptide was synthesized using standard Fmoc chemistry.

DCM was added to the vessel containing CTC Resin (7.00 mmol, 12.92 g, 0.65 mmol/g) and Fmoc-Gly-Gly —OH (2.48 g, 7.00 mmol, 1.00 equiv.) with N₂ bubbling.

DIEA (4.0 equiv.) was added dropwise and mixed for 2 hrs.

MeOH (13.0 mL) was added and mixed for 30 mins.

The reaction mixture was drained and washed with DMF for 5 times. 20% piperidine/DMF was added and mixed for 30 mins.

The resultant was drained and then washed with DMF for 30 seconds for 5 times.

Fmoc-amino acid solution was added and mixed 30 seconds, and then activation bufferwas added, followed by N₂ bubbling for about 1 hr. 20% piperidine/DMF was added and mixed for 30 mins.

Steps 4 to 8 were repeated for the subsequent amino acid coupling step.

The coupling reaction was monitored by ninhydrin test, and the resin was washed with DMF for 5 times.

The resin was washed with MeOH for 3 times and dried by vacuum.

### Step b: Peptide Cleavage

Cleavage buffer (20% HFIP/DCM) was added to the flask containing the side chain protected peptide at room temperature and stirred for 30 mins twice.

The reaction mixture was filtered and the solvent was removed under vacuum.

Compound 10 (2.70 g,crude) was obtained as a white solid.

### Step c: General Procedure for Preparation of Compound 10

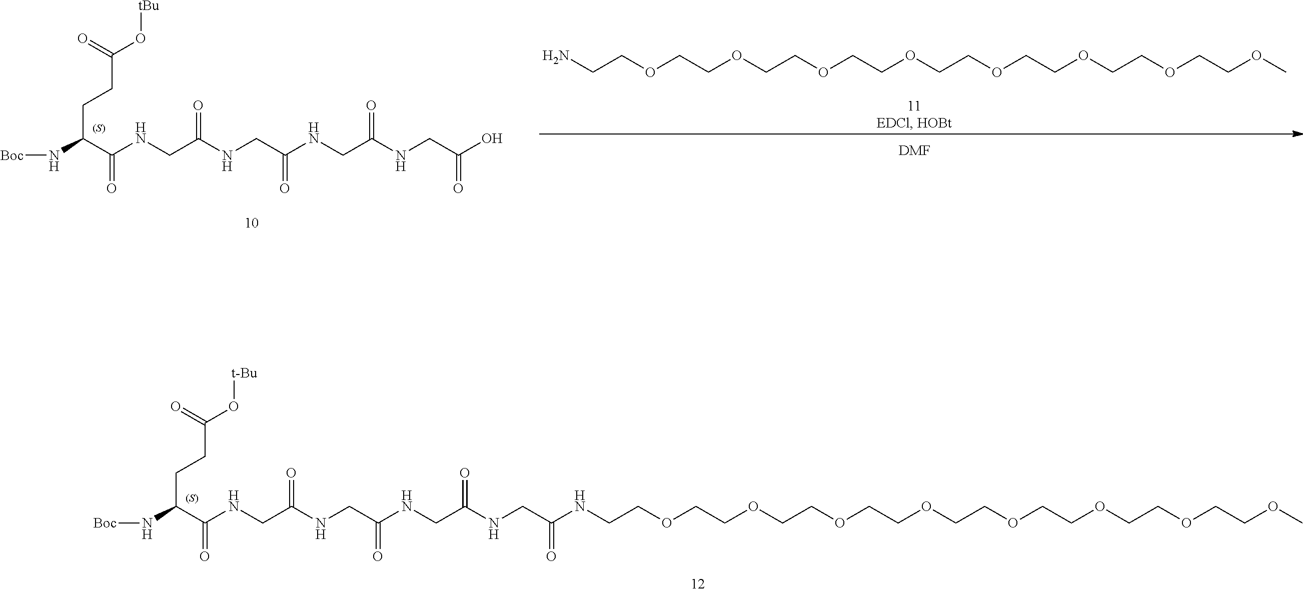

To a mixture of (S)-(S)—(S)-(S)-(5-(tert-butoxy)-2-((tert-butoxycarbonyl)amino)-5-oxopentanoyl)glycylglycylglycylglycine (1.1 g, 2.07 mmol, 1.00 equiv.) and 2,5,8,11,14,17,20,23-octaoxapentacosan-25-amine (872.92 mg, 2.28 mmol, 1.10 equiv.) in DMF (10 mL) EDCI (793.42 mg, 4.14 mmol, 2.00 equiv.) and HOBt (559.25 mg, 4.14 mmol, 2.00 equiv.) were added in one portion at 25° C. and reacted for 12 hrs. LCMS showed that the reaction was completed. The solvent was removed under reduced pressure to afford tert-butyl (S)-40-((tert-butoxycarbonyl)amino)-27,30,33,36,39-pentaoxo-2,5,8,11,14,17,20,23-octaoxa-26,29,32,35,38-pentaazatritetracontan-43-oate (1.50 g, 1.62 mmol, 78.38% yield, 97% purity) as a colorless solid.

### Step d: General Procedure for Preparation of Compound 13

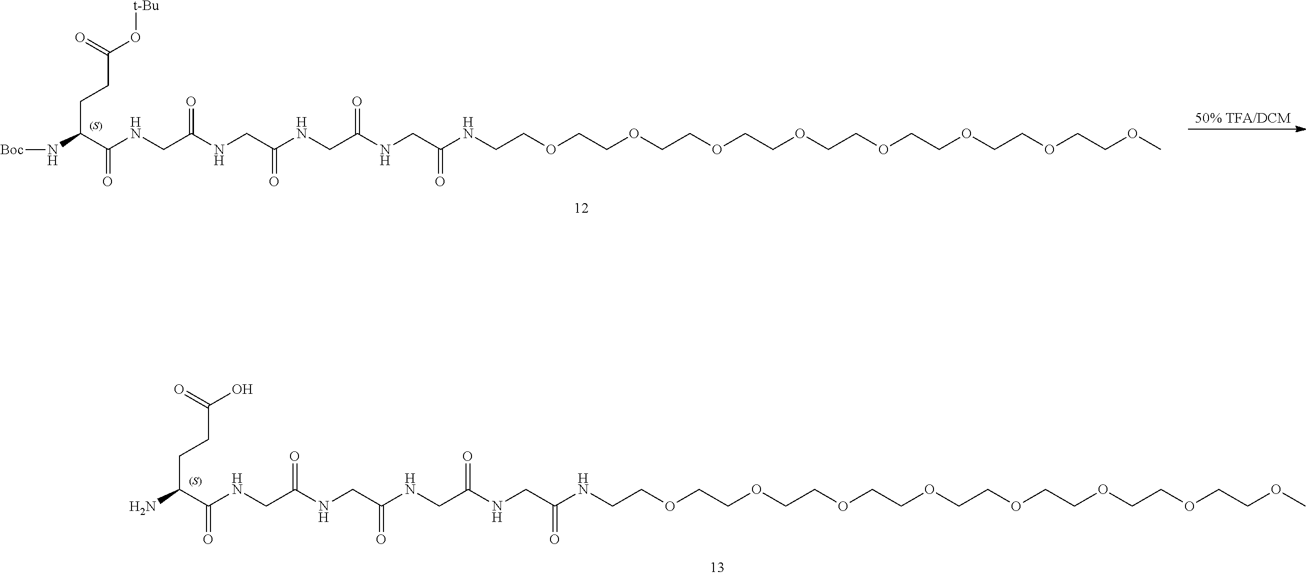

To a mixture of tert-butyl (S)-40-((tert-butoxycarbonyl)amino)-27,30,33,36,39-pentaoxo-2,5,8,11,14,17,20,23-octaoxa-26,29,32,35,38-pentaazatritetracontan-43-oate (1.50 g, 1.67 mmol, 1.00 equiv.) in DCM (5 mL) TFA (5 mL) was added in one portion at 25° C. and the mixture was stirred for 30 mins. LCMS showed that the reaction was completed. The solvent was removed under reduced pressure to afford (S)-40-amino-27,30,33,36,39-pentaoxo-2,5,8,11,14,17,20,23-octaoxa-26,29,32,35,38-pentaazatritetracontan-43-oic acid (1.15 g, 1.52 mmol, 90.98% yield, 98% purity) as colorless solid.

### Step e: General procedure for preparation of 372

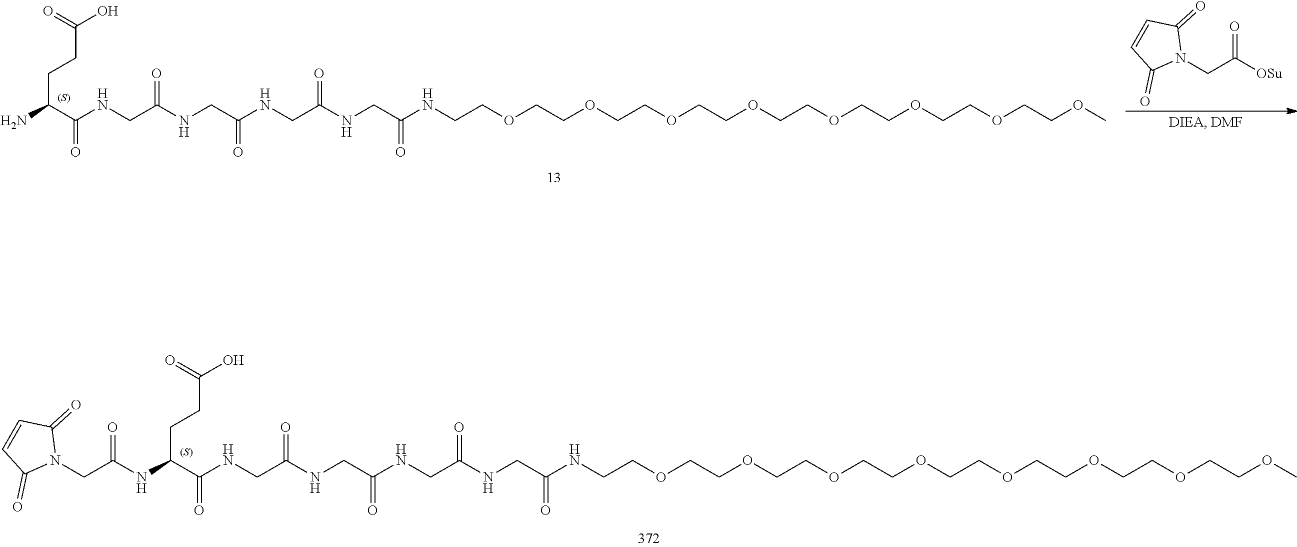

To a mixture of (S)-40-amino-27,30,33,36,39-pentaoxo-2,5,8,11,14,17,20,23-octaoxa-26,29,32,35,38-pentaazatritetracontan-43-oic acid (580.0 mg, 782.94 umol, 1.00 equiv.) and (2,5-dioxopyrrolidin-1-yl) 2-(2,5-dioxopyrrol-1-yl)acetate (296.16 mg, 1.17 mmol, 1.50 equiv.) in DMF (6 mL) DIEA (303.57 mg, 2.35 mmol, 409.12 μL, 3.00 equiv.) was added in one portion at 0° C. and reacted for 1 hr. LCMS showed that the reaction was completed. The mixture was purified by prep-HPLC (A: 0.075% TFA in H₂O, B: ACN) to afford (S)-40-(2-(2,5-dioxo-2,5-dihydro-1H-pyrrol-1-yl)acetamido)-27,30,33,36,39-pentaoxo-2,5,8,11,14,17,20,23-octaoxa-26,29,32,35,38-pentaazatritetracontan-43-oic acid (235.0 mg, 261.80 umol, 33.44% yield, 97.8% purity) as a colorless solid.

### Example III-8 Synthesis of Linker Compound 391

### Step a: General Procedure for Preparation of Compound 5

### Solid Phase Peptide Synthesis:

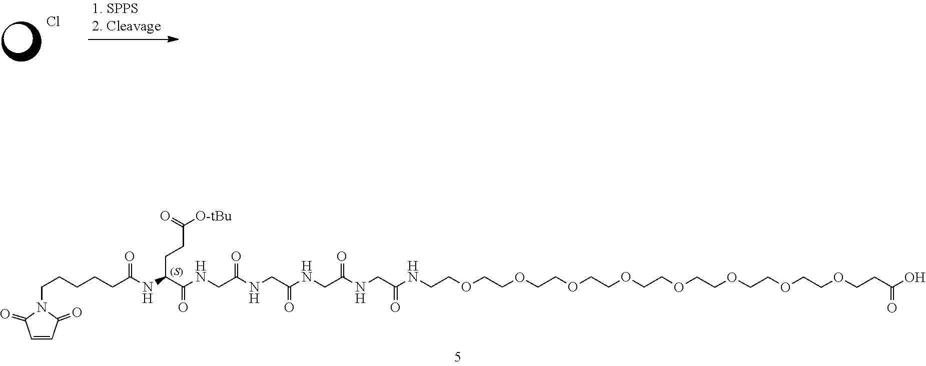

The peptide was synthesized using standard Fmoc chemistry.

Resin preparation: To a vessel containing CTC resin (2.0 mmol, 2.0 g, 1.00 mmol/g) and Fmoc-PEG₈-OH (2.0 mmol, 1.326 g, 1.0 equiv.) in DCM (20 mL) DIEA (4.0 equiv.), N₂ was bubbled for 2 hrs at 15° C. MeOH (2.0 mL) was added and mixed for 30 min. The mixture was drained and washed with DMF for 5 times. Then 20% piperidine in DMF (20 mL) was added and the mixture was bubbled with N₂ for 30 mins at 15° C. Then the mixture was filtered to obtain the resin. The resin was washed with DMF (40 mL)*5 before proceeding to the next step.

Coupling: A solution of Fmoc-Gly-OH (3.00 equiv.), HBTU (2.85 equiv.) and DIEA (6.00 equiv.) in DMF (20 mL) was added to the resin with N₂ bubbling for 30 min at 15° C. The coupling reaction was monitored by ninhydrin test, if it showed colorless, the coupling was completed. The resin was then washed with DMF (40 mL)*5.

De-protection: 20% piperidine in DMF (40 mL) was added to the resin and the mixture was bubbled with N₂ for 30 min at 15° C. The resin was then washed with DMF (40 mL)*5. The De-protection reaction was monitored by ninhydrin test, if it showed blue or other brownish red, the reaction was completed.

Steps 2 and 3 were repeated for all other amino acids: (2-5 in Table 1).

After the last position completed, the resin was washed with DMF (40 mL)*5, MeOH (40 mL)*5 and then dried under vacuum.

Note: Synthesis scale: 2.0 mmol

### Step b: Peptide Cleavage and Purification

Cleavage buffer (20% HFIP/DCM) was added to the flask containing the side chain protected peptide at room temperature and stirred for 1.0 hr.

The reation mixture was filtered and the filtrate was collected. The mixture was concentrated under reduced pressure.

The residue was dissolved in MeCN (150 ml) and H₂O (150 ml), then lyophilized to afford (S)-42-(3-(tert-butoxy)-3-oxopropyl)-49-(2,5-dioxo-2,5-dihydro-1H-pyrrol-1-yl)-29,32,35,38,41,44-hexaoxo-4,7,10,13,16,19,22,25-octaoxa-28,31,34,37,40,43-hexaazanonatetracontanoic acid (1.8 g, crude) as a white solid.

### Step c: General Procedure for Preparation of Compound 3

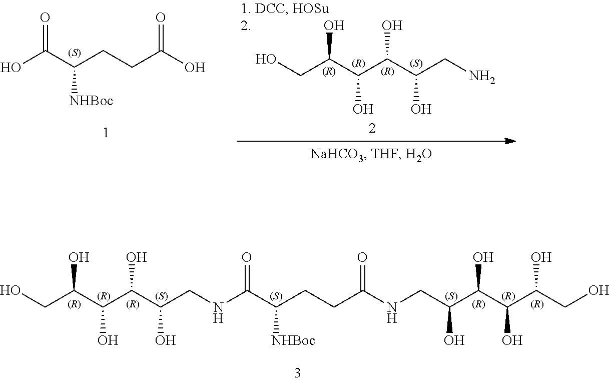

A mixture of (tert-butoxycarbonyl)-L-glutamic acid (11.0 g, 44.5 mmol, 1.00 equiv.), DCC (18.4 g, 88.9 mmol, 2.00 equiv,), DMAP (1.09 g, 8.90 umol, 0.20 equiv.) and HOSu (20.5 g, 177.9 mmol, 4.00 equv.) was dissolved with THF (400 mL), the reaction mixture was stirred at 0° C. for 0.5 h then filtered to remove undissolved residue. The filter was added to a solution of (2R,3R,4R,5S)-6-aminohexane-1,2,3,4,5-pentaol (24.2 g, 133.5 mmol, 3.00 equiv.) and NaHCO₃ (7.47 g, 88.9 mmol, 2.00 equiv.) in H₂O (200 mL), then the reaction mixture was stirred at 25° C. for 1.5 h. LC-MS showed the reaction was complete. The reaction mixture was filtered and concentrated under reduced pressure to give a residue. The crude product was purified by prep-HPLC (TFA condition) to give tert-butyl ((S)-1,5-dioxo-1,5-bis(((2S,3R,4R,5R)-2,3,4,5,6-pentahydroxyhexyl)amino)pentan-2-yl)carbamate (6.40 g, 11.2 mmol, 95.2% purity, 25.1% yield) as a white solid.

### Step d: General Procedure for Preparation of Compound 4

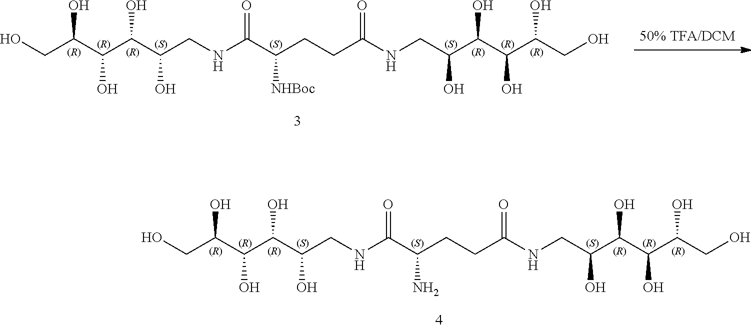

A mixture of tert-butyl ((S)-1,5-dioxo-1,5-bis(((2S,3R,4R,5R)-2,3,4,5,6-pentahydroxyhexyl)amino)pentan-2-yl)carbamate (6.12 g, 10.6 mmol, 1.00 equiv.) and TFA (46.2 g, 405.2 mmol, 30.0 mL, 37.9 equiv.) was dissolved with DCM (30 mL) and then the reaction mixture was stirred at 20° C. for 1 h. LC-MS showed the reaction was complete. The reaction mixture was filtered and concentrated under reduced pressure to give (S)-2-amino-N₁,N₅-bis((2S,3R,4R,5R)-2,3,4,5,6-pentahydroxyhexyl)pentanediamide (2.33 g, 11.2 mmol, 95.2% purity, 25.1% yield) as a white solid.

### Step f: General Procedure for Preparation of Compound 6

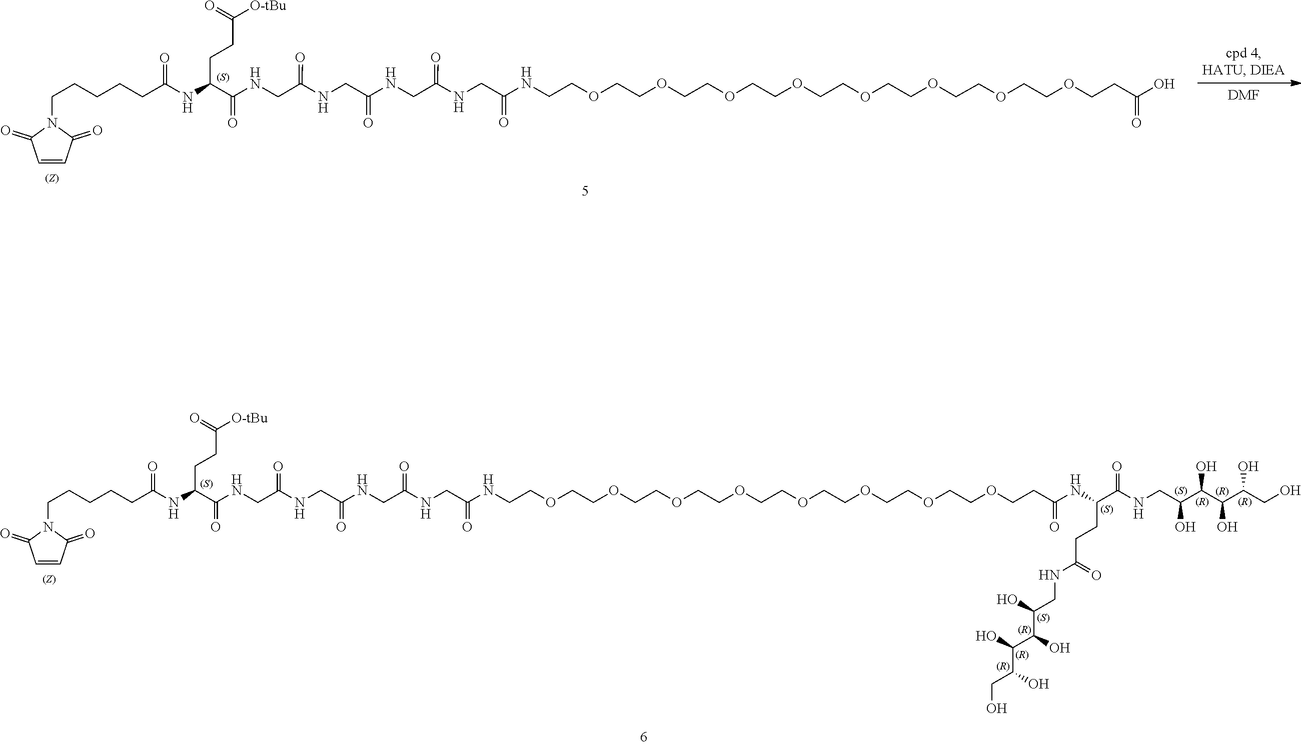

To a solution of (S)-42-(3-(tert-butoxy)-3-oxopropyl)-49-(2,5-dioxo-2,5-dihydro-1H-pyrrol-1-yl)-29,32,35,38,41,44-hexaoxo-4,7,10,13,16,19,22,25-octaoxa-28,31,34,37,40,43-hexaazanonatetracontanoic acid (400 mg, 381.6 umol, 1.00 equiv.) and (S)-2-amino-N₁,N₅-bis((2S,3R,4R,5R)-2,3,4,5,6-pentahydroxyhexyl)pentanediamide (271.0 mg, 572.5 umol, 1.50 equiv.) in DMF (0.5 mL), HATU (145.1 mg, 381.6 umol, 1.00 equiv.) and DIEA (147.9 mg, 1.14 mmol, 199.4 μL, 3.00 equiv.) were added and then the reaction mixture was stirred at 20° C. for 0.5 h. LC-MS showed the reaction was complete. The reaction mixture was purified by prep-HPLC (TFA condition) to give tert-butyl (4S,47S,53S,54R,55R,56R)-4-(6-(2,5-dioxo-2,5-dihydro-1H-pyrrol-1-yl)hexanamido)-53,54,55,56,57-pentahydroxy-5,8,11,14,17,45,50-heptaoxo-47-(((2S,3R,4R,5R)-2,3,4,5,6-pentahydroxyhexyl)carbamoyl)-21,24,27,30,33,36,39,42-octaoxa-6,9,12,15,18,46,51-heptaazaheptapentacontanoate (306.0 g, 203.5 umol, 94.8% purity, 53.3% yield) as a white solid.

### Step g: General procedure for preparation of 391

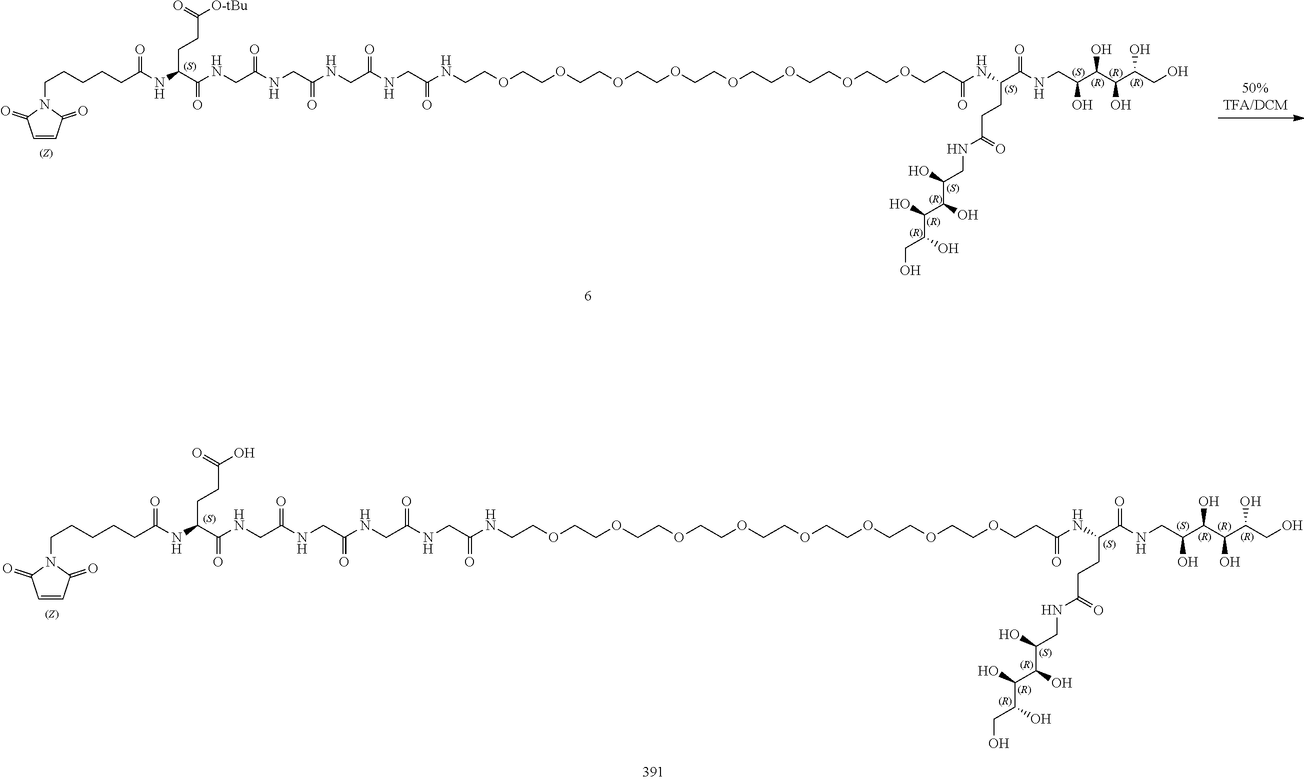

A mixture of tert-butyl (4S,47S,53S,54R,55R,56R)-4-(6-(2,5-dioxo-2,5-dihydro-1H-pyrrol-1-yl)hexanamido)-53,54,55,56,57-pentahydroxy-5,8,11,14,17,45,50-heptaoxo-47-(((2S,3R,4R,5R)-2,3,4,5,6-pentahydroxyhexyl)carbamoyl)-21,24,27,30,33,36,39,42-octaoxa-6,9,12,15,18,46,51-heptaazaheptapentacontanoate (300.0 mg, 199.5 umol, 1.00 equiv.) and TFA (4.62 g, 40.5 mmol, 3 mL, 203.1 equiv.) was dissolved with DCM (3 mL) then the reaction mixture was stirred at 20° C. for 0.5 h. LC-MS showed the reaction was complete. The reaction mixture was filtered and concentrated under reduced pressure. The residue was purified by prep-HPLC (TFA condition) to give (4S,47S,53S,54R,55R,56R)-4-(6-(2,5-dioxo-2,5-dihydro-1H-pyrrol-1-yl)hexanamido)-53,54,55,56,57-pentahydroxy-5,8,11,14,17,45,50-heptaoxo-47-(((2S,3R,4R,5R)-2,3,4,5,6-pentahydroxyhexyl)carbamoyl)-21,24,27,30,33,36,39,42-octaoxa-6,9,12,15,18,46,51-heptaazaheptapentacontanoic acid (203.0 mg, 133.7 umol, 66.7% yield, 95.3% purity) as a white solid.

### Example III-8 Synthesis of Linker Compound 392

392 were synthesized using the same method as 391.

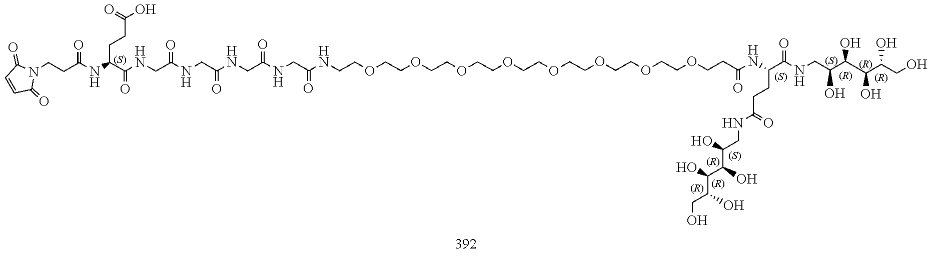

### Example III-9 Synthesis of linker compound M79-0

### Step a:

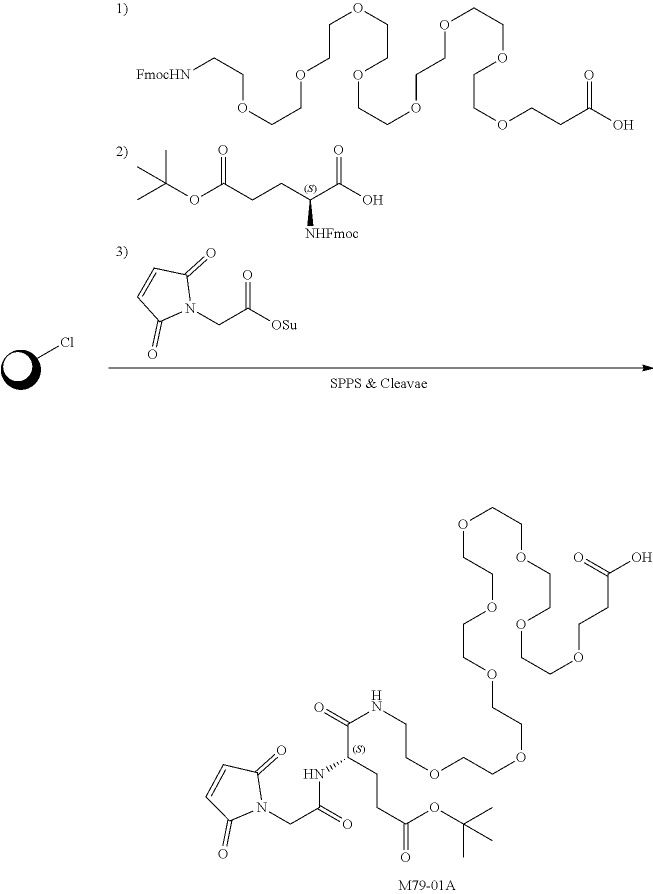

Trt-Cl (3.0 g, 3.3 mmol) and dichloromethane (60 mL) were added at room temperature. The Trt-Cl resin (3 g, 3.3 mmol) was swelling for 30 min. After filtration, the Trt-Cl resin was washed with N,N-dmethylformamide (3×60 mL). Then N,N-dmethylformamide (60 mL), 1-{[(9H-fluoren-9-ylmethoxy)carbonyl]amino}-3,6,9,12,15,18,21,24-octaoxaheptacosan-27-oic acid (4.40 g, 6.631 mmol, 0.69 equiv) and N,N-diisopropylethylamine (4.26 g, 32.958 mmol, 3.43 equiv) was added in sequence. Then the column was bubbled with nitrogen atmosphere for 2 h. After filtration, the residue was washed with N,N-dmethylformamide (3×60 mL), 60 mL dichloromethane/methanol/N,N-diisopropylethylamine (80/15/5) for 30 min, methanol (3×60 mL), dichloromethane (3×60 mL). The residue was dried by nitrogen atmosphere for 2h. 20% piperdine (60 mL in N,N-dmethylformamide) was added to the above residue at room temperature. The resulting mixture was stirred for lh at room temperature. After filtration, the residue was washed with N,N-dmethylformamide (3×60 mL). Then (2S)-5-(tert-butoxy)-2-{[(9H-fluoren-9-ylmethoxy)carbonyl]amino}-5-oxopentanoic acid (3.56 g, 8.366 mmol, 2 equiv), N,N-dmethylformamide (60 mL), N,N-diisopropylethylamine (4.32 g, 33.464 mmol, 8 equiv) and 2-(7-Azabenzotriazol-1-yl)-N,N,N′,N′-tetramethyluronium hexafluorophosphate (6.36 g, 16.732 mmol, 4 equiv) was added in sequence. Then the column was bubbled with nitrogen atmosphere for 2 h. After filtration, the residue was washed with N,N-dmethylformamide (3×60 mL). 20% piperdine (60 mL in N,N-dmethylformamide) was added to the above residue at room temperature. The resulting mixture was stirred for lh at room temperature. After filtration, the residue was washed with N,N-dmethylformamide (3×60 mL). Then 2,5-dioxopyrrolidin-1-yl-2-(2,5-dioxopyrrol-1-yl)acetate (1.5 g, 5.994 mmol, 1 equiv), N,N-dmethylformamide (60 mL), and triethylamine (3.01 g, 29.746 mmol, 5 equiv) was added in sequence. Then the column was bubbled with nitrogen atmosphere for 2 h. After filtration, the residue was washed with N,N-dmethylformamide (3×60 mL), dichloromethane (3×60 mL). Then 20 mL 20% 1,1,1,3,3,3-Hexafluoro-2-propanol in dichloromethane (60 ml) was added and bubbled with nitrogen atmosphere for 2 h. The resulting mixture was concentrated under reduced pressure. There was 1-[(2S)-5-(tert-butoxy)-2-[2-(2,5-dioxopyrrol-1-yl)acetamido]-5-oxopentanamido]-3,6,9,12,15, 18,21,24-octaoxaheptacosan-27-oic acid (2.4 g) obtained as a brown oil. LC-MS (ES, m/z): [M+H]=764.5. ¹H NMR (300 MHz, DMSO-d₆) δ 8.36 (d, J=8.2 Hz, 2H), 7.98-8.02 (m, 2H), 7.08 (d, J=1.6 Hz, 2H), 5.75 (d, J=1.7 Hz, 2H), 5.12-5.16 (m, 3H), 4.20-4.26 (m, 2H), 4.10 (d, J=2.2 Hz, 2H), 3.60 (t, J=6.5 Hz, 4H), 3.41 (t, J=5.8 Hz, 4H), 3.20-3.23 (m, 4H), 2.44 (t, J=6.3 Hz, 3H), 2.18 (t, J=8.0 Hz, 3H), 1.8-1.67 (m, 4H), 1.39 (d, J=1.7 Hz, 12H).

### Step b:

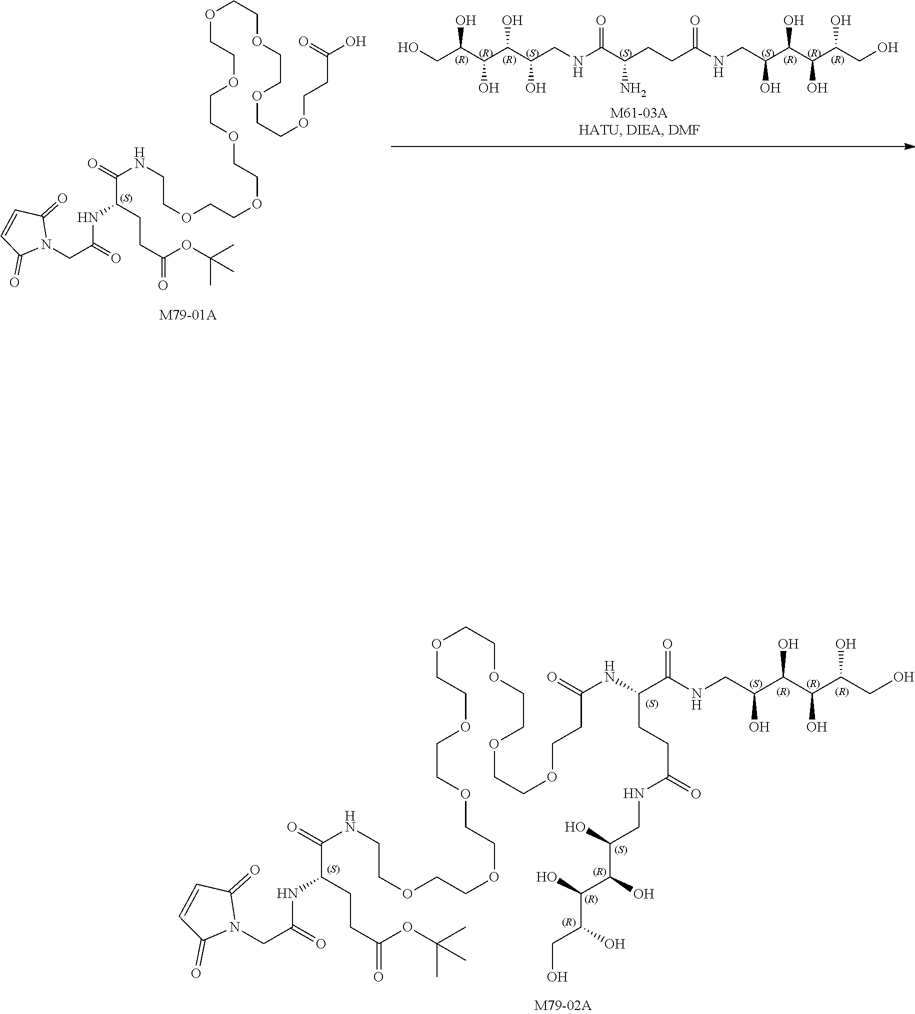

1-[(2S)-5-(tert-butoxy)-2-[2-(2,5-dioxopyrrol-1-yl)acetamido]-5-oxopentanamido]-3,6,9,12,15,18,21,24-octaoxaheptacosan-27-oic acid (1 g, 1.309 mmol, 1 equiv), (2S)-2-amino-N,N′-bis[(2S,3R,4R,5R)-2,3,4,5,6-pentahydroxyhexyl]pentanediamide (0.54 g, 1.139 mmol, 1 equiv), N,N-diisopropylethylamine (0.62 g, 1.309 mmol, 1 equiv), N,N-dmethylformamide (20 ml) and 2-(7-Azabenzotriazol-1-yl)-N,N,N′,N′-tetramethyluronium hexafluorophosphate (0.75 g, 1.963 mmol, 1.5 equiv) were added at room temperature.

The resulting mixture was stirred for lh at room temperature. The residue was purified by reverse flash chromatography with the following conditions: column, C18 gel; mobile phase, acetonitrile in water (0.1% trifluoroacetic acid), 10% to 40% gradient in 20 min; detector, UV 200 nm. There was tert-butyl (4S)-4-[(26-{[(1S)-1,3-bis({[(2S,3R,4R,5R)-2,3,4,5,6-pentahydroxyhexyl]carbamoyl})propyl]carbamoyl}-3,6,9,12,15,18,21,24-octaoxahexacosan-1-yl)carbamoyl]-4-[2-(2,5-dioxopyrrol-1-yl)acetamido]butanoate (700 mg, 43.85%) obtained as a brown oil. LC-MS: (ES, m/z): [M+2H]²=610.6. ¹H NMR (300 MHz, DMSO-d₆) δ 8.35 (d, J=8.2 Hz, 1H), 8.02 (d, J=8.6 Hz, 2H), 7.71 (d, J=14.2 Hz, 2H), 7.09 (d, J=1.7 Hz, 2H), 4.22 (m, 3H), 4.10 (s, 2H), 3.70-3.40 (m, 50H), 3.20-3.26 (m, 4H), 3.18 (s, 2H), 3.02-3.06 (m, 2H), 2.39 (s, 2H), 2.18 (t, J=8.0 Hz, 2H), 2.10 (t, J=7.9 Hz, 2H), 1.80-1.86 (m, 2H), 1.68-1.73 (m, 2H), 1.39 (d, J=1.7 Hz, 9H).

### Step c:

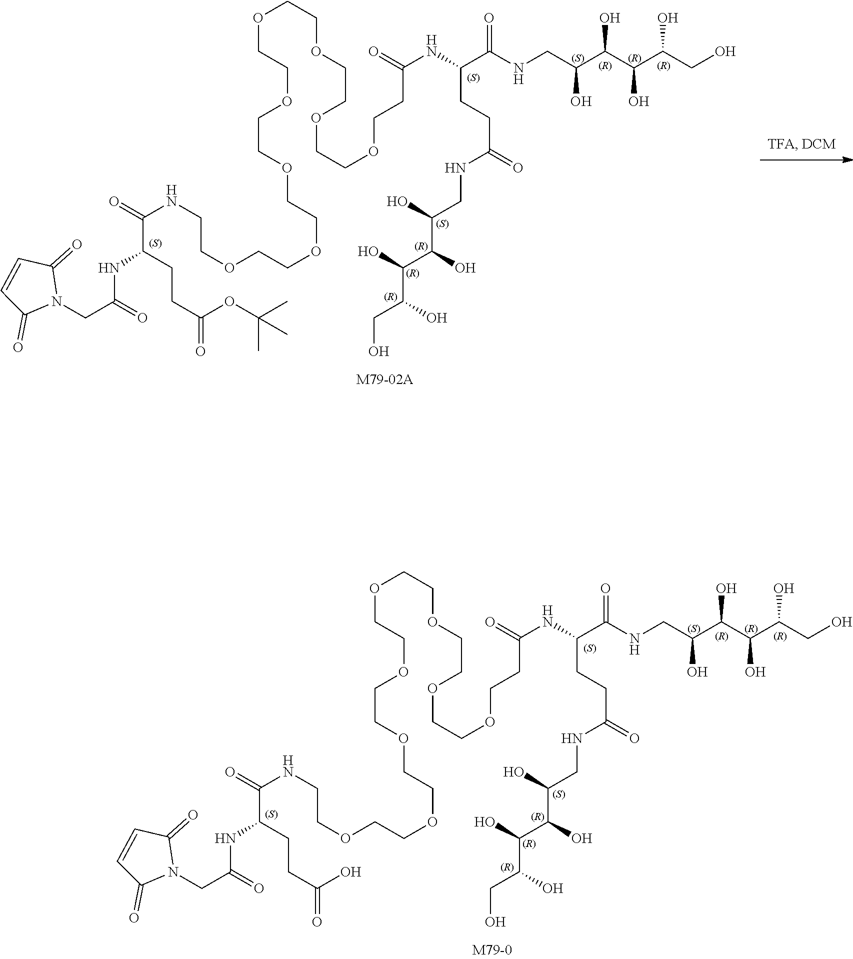

Tert-butyl(4S)-4-[(26-{[(1S)-1,3-bis({[(2S,3R,4R,5R)-2,3,4,5,6-pentahydroxyhexyl]carbamoyl}) propyl]carbamoyl}-3,6,9,12,15,18,21,24-octaoxahexacosan-1-yl)carbamoyl]-4-[2-(2,5-dioxopyrrol-1-yl)acetamido]butanoate (700 mg, 0.574 mmol, 1 equiv), dichloromethane (10 mL) and trifluoroacetic acid (10 mL) was added at room temperature. The resulting mixture was stirred for 30 min at room temperature. The resulting mixture was concentrated under reduced pressure. The crude product (600 mg) was purified by Prep-HPLC with the following conditions (Column: XBridge Prep OBD C18 Column, 30*150 mm, 5 μm ; Mobile Phase A: Water(0.05% trifluoroacetic acid ), Mobile Phase B: acetonitrile; Flow rate: 60 mL/min; Gradient: 3% B to 15% B in 9 min, 15% B; Wave Length: 220 nm; RTl(min): 7; Number Of Runs: 0) to afford (4S)-4-[(26-{[(1S)-1,3-bis({[(2S,3R,4R,5R)-2,3,4,5,6-pentahydroxyhexyl]carbamoyl})propyl]carbamoyl}-3,6,9,12,15,18,21,24-octaoxahexacosan-1-yl)carbamoyl]-4-[2-(2,5-dioxopyrrol-1-yl)acetamido]butanoic acid (220.2 mg, 32.48%) as a colorless semi-solid. LC-MS (ES, m/z): [M+H]⁺=1163.45. ¹H NMR (300 MHz, Deuterium Oxide) 6 6.87 (s, 2H), 4.23-4.26 (m, 4H), 3.79-3.50 (m, 45H), 3.30-3.35 (m, 4H), 3.20-3.26 (m, 2H), 2.52 (s, 2H), 2.41 (t, J=7.4 Hz, 2H), 2.29 (t, J=7.7 Hz, 2H), 2.05 (d, J=10.7 Hz, 3H), 1.89 (dt, J=14.4, 7.4 Hz, 2H).

### Compound-Linker Constructs

### Example IV-1 Synthesis of Linker-STING Agonist Compound LS1

### Step a:

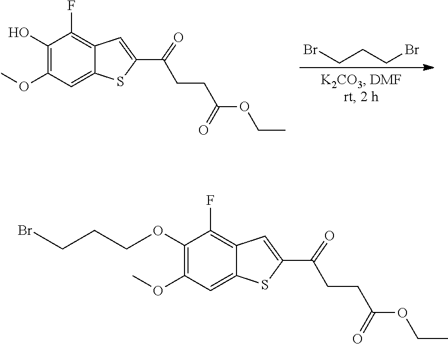 [SMILES: CCOC(=O)CCC(=O)c1cc2c(F)c(O)c(OC)cc2s1 ; BrCCCBr ; CCOC(=O)CCC(=O)c1cc2c(F)c(OCCCBr)c(OC)cc2s1]

1,3-Dibromopropane (2.9835 g, 14.7780 mmol) was added to a solution of ethyl 4-(4-fluoro-5-hydroxy-6-methoxybenzo[b]thiophen-2-yl)-4-oxobutanoate (0.863 g, 2.6445 mmol) and K₂CO₃ (1.081 g, 7.8217 mmol) in DMF (20 mL) at 21° C. The reaction mixture was stirred for 2 h at 25° C. The resulting reaction mixture was diluted with EA (150 mL), and then washed with water (200 mL) and brine (2×150 mL). The organic layer was collected and dried over Na₂SO₄, filtered and evaporated under reduced pressure. The residue was purified by silica gel column chromatography, eluting with ethyl acetate/n-hexane (0-50%). The purified fraction was concentrated and dried under reduced pressure. There was ethyl 4-[5-(3-bromopropoxy)-4-fluoro-6-methoxy-benzothiophen-2-yl]-4-oxo-butanoate (1.12 g, 94.6811% yield) obtained as a gray white solid. LCMS: (ESI, m/z): [M+H]⁺=447.000. ¹H NMR (400 MHz, CDCl₃) δ 8.00 (s, 1H), 7.10 (s, 1H), 4.24 (t, J=5.6 Hz, 2H), 4.19 (q, J=7.1 Hz, 2H), 3.97 (s, 3H), 3.74 (t, J=6.4 Hz, 2H), 3.35 (t, J=6.7 Hz, 2H), 2.80 (t, J=6.7 Hz, 2H), 2.38-2.26 (m, 2H), 1.29 (t, J=7.1 Hz, 3H).

### Step b:

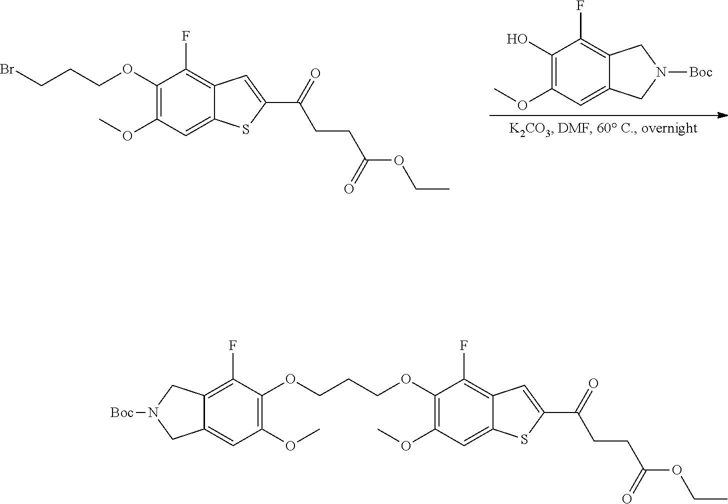 [SMILES: CCOC(=O)CCC(=O)c1cc2c(F)c(OCCCBr)c(OC)cc2s1 ; COc1cc2c(c(F)c1O)CN(C(=O)OC(C)(C)C)C2 ; CCOC(=O)CCC(=O)c1cc2c(F)c(OCCCOc3c(OC)cc4c(c3F)CN(C(=O)OC(C)(C)C)C4)c(OC)cc2s1]

K₂CO₃ (0.92 g, 6.6568 mmol) was added to a solution of tert-butyl 4-fluoro-5-hydroxy-6-methoxyisoindoline-2-carboxylate (0.72 g, 2.5415 mmol) and ethyl 4-(5-(3-bromopropoxy)-4-fluoro-6-methoxybenzo[b]thiophen-2-yl)-4-oxobutanoate (1.11 g, 2.4815 mmol) in DMF (20 mL) at 20° C. The reaction mixture was heated to 60° C. and stirred overnight. The resulting reaction mixture was diluted with EA (150 mL), and then washed with water (200 mL) and brine (2×150 mL). The organic layer was collected and dried over Na₂SO₄, filtered and evaporated under reduced pressure. The residue was purified by silica gel column chromatography, eluting with ethyl acetate/n-hexane (0-80%). The purified fraction was concentrated and dried under reduced pressure. There was tert-butyl 5-[3-[2-(4-ethoxy-4-oxo-butanoyl)-4-fluoro-6-methoxy-benzothiophen-5-yl]oxypropoxy]-4-fluoro-6-methoxy-isoindoline-2-carboxylate (0.81 g, 49.0546% yield) obtained as a slight yellow semi-solid. LCMS: (ESI, m/z): [M+H-Boc]⁺=550.200. ¹H NMR (400 MHz, CDCl₃) δ 8.00 (s, 1H), 7.08 (s, 1H), 6.59 (s, 1H), 4.64 (br.-s, 4H), 4.37 (t, J=6.1 Hz, 2H), 4.31 (t, J=6.0 Hz, 2H), 4.19 (q, J=7.1 Hz, 2H), 3.94 (s, 3H), 3.83 (s, 3H), 3.35 (t, J=6.7 Hz, 2H), 2.80 (t, J=6.7 Hz, 2H), 2.29 -2.17 (m, 2H), 1.53 (s, 9H), 1.28 (t, J=7.1 Hz, 3H).

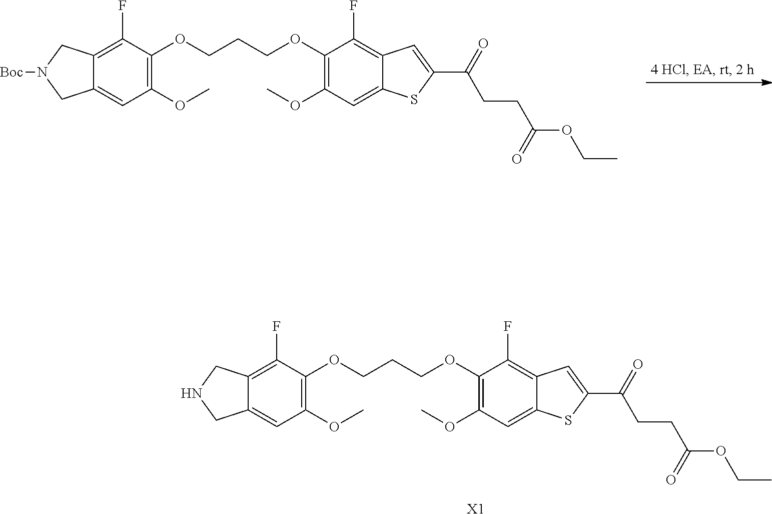 [SMILES: CCOC(=O)CCC(=O)c1cc2c(F)c(OCCCOc3c(OC)cc4c(c3F)CN(C(=O)OC(C)(C)C)C4)c(OC)cc2s1 ; CCOC(=O)CCC(=O)c1cc2c(F)c(OCCCOc3c(OC)cc4c(c3F)CNC4)c(OC)cc2s1 ; "X1"]

Tert-butyl 5-[3-[2-(4-ethoxy-4-oxo-butanoyl)-4-fluoro-6-methoxy-benzothiophen-5-yl]oxypropoxy]-4-fluoro-6-methoxy-isoindoline-2-carboxylate (0.323 g, 497.1542 mol) was dissolved in 4 N HCl solution (15 mL) in EA at 18° C. The reaction mixture was stirred for 2 h at 18° C. The resulting reaction mixture was concentrated under reduced pressure. There was ethyl-4-[4-fluoro-5-[3-(4-fluoro-6-methoxy-isoindolin-5-yl)oxypropoxy]-6-methoxy-benzothiophen-2-yl]-4-oxo-butanoate (0.288 g, 98.8489% yield, HCl salt) obtained as a brown solid. LCMS: (ESI, m/z): [M+H]=550.150. ¹H NMR (400 MHz, DMSO-d₆) δ 9.92 (s, 2H), 8.32 (s, 1H), 7.58 (s, 1H), 6.96 (s, 1H), 4.50 (s, 2H), 4.46 (s, 2H), 4.30-4.16 (m, 4H), 4.06 (q, J=7.1 Hz, 2H), 3.88 (s, 3H), 3.78 (s, 3H), 3.38 (t, J=6.2 Hz, 2H), 2.66 (t, J=6.2 Hz, 2H), 2.11-2.01 (m, 2H), 1.18 (t, J=7.1 Hz, 3H).

### Step d:

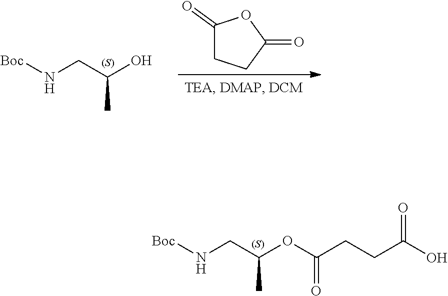

Succinic anhydride (1.23 g, 12.2911 mmol) was added to a mixed solution of tert-butyl (S)-(2-hydroxypropyl)carbamate (2.06 g, 11.7563 mmol), TEA (2.3296 g, 23.0222 mmol), and DMAP (0.054 g, 442.0179 mol) in DCM (30 mL) at 20° C. The reaction mixture was stirred for 4 h at 25° C. The resulting reaction mixture was concentrated under reduced pressure. There was 4-[(1S)-2-(tert-butoxycarbonylamino)-1-methyl-ethoxy]-4-oxo-butanoic acid (3.08 g, 95.1649% yield) obtained as a colorless oil, which was directly useded in the next step. LCMS: (ESI, m/z): [M−H]⁻=274.100.

### Step e:

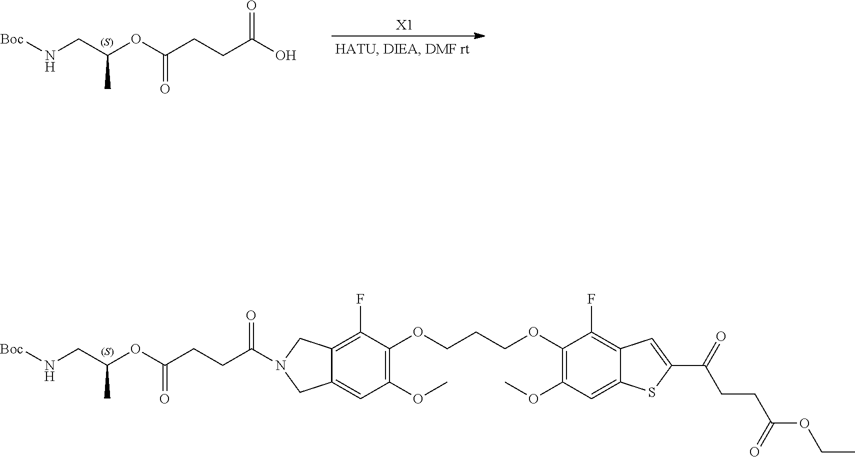

HATU (0.290 g, 762.6977 mol) was added to a solution of 4-[(1S)-2-(tert-butoxycarbonylamino)-1-methyl-ethoxy]-4-oxo-butanoic acid (0.189 g, 686.5303 mol) and DIEA (385 μL, 2.2103 mmol) in DMF (12 mL) at 18° C. After stirring for 15 min, ethyl-4-[4-fluoro-5-[3-(4-fluoro-6-methoxy-isoindolin-5-yl)oxypropoxy]-6-methoxy-benzothiophen-2-yl]-4-oxo-butanoate (0.258 g, 440.2405 mol) was added. The reaction mixture was stirred for 1.5 h at 25° C. The resulting reaction mixture was diluted with EA (100 mL), and then washed with brine (2×100 mL). The organic layer was collected and dried over Na₂SO₄, filtered and evaporated under reduced pressure. The residue was purified by Prep-TLC with the developing solvent of DCM/MeOH (25:1). There was ethyl 4-[5-[3-[2-[4-[(1S)-2-(tert-butoxycarbonylamino)-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.288 g, 81.0778% yield) obtained as a brown solid. LCMS: (ESI, m/z): [M+H-Boc]=707.250.

### Step f:

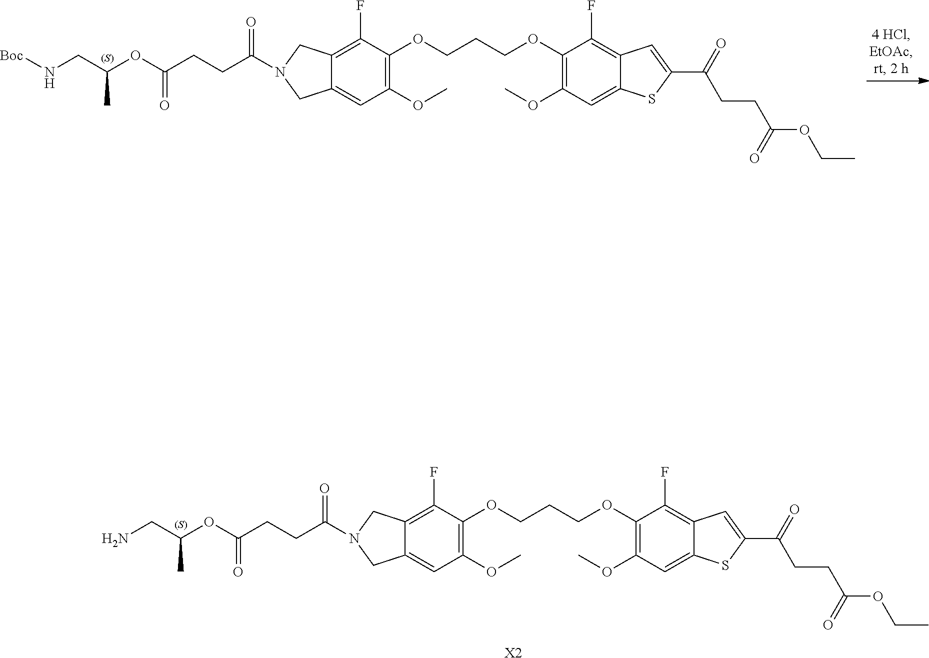

Ethyl-4-[5-[3-[2-[4-[(1S)-2-(tert-butoxycarbonylamino)-1-methyl-ethoxy]-4- oxo-butanoyl]-4-fluoro-6-methoxy-isoindolin-5-yl]oxypropoxy]-4-fluoro-6-methoxy-benzothiophen-2-yl]-4-oxo-butanoate (0.235 g, 291.2509 mol) was dissolved in 4 N HCl solution (10 mL) in EA at 20° C. The reaction mixture was stirred for 2 h at 20° C. The resulting reaction mixture was concentrated under reduced pressure, and then lyophilized overnight. There was ethyl 4-[5-[3-[2-[4-[(1S)-2-amino-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (232 mg, 93.2456% yield, HCl salt) obtained as a brown solid. LCMS: (ESI, m/z): [M+H]⁺=707.250. ¹H NMR (400 MHz, DMSO-d₆) δ 8.31 (s, 1H), 8.13 (s, 3H), 7.57 (s, 1H), 6.90 (s, 0.5H), 6.88 (s, 0.5H), 5.04-4.95 (m, 1H), 4.80 (s, 2H), 4.57 (s, 2H), 4.30-4.15 (m, 4H), 4.06 (q, J=7.1 Hz, 2H), 3.88 (s, 3H), 3.78 (s, 3H), 3.37 (t, J=6.3 Hz, 2H), 3.09-2.89 (m, 2H), 2.72-2.55 (m, 6H), 2.10-1.99 (m, 2H), 1.24-1.15 (m, 6H).

### Step g:

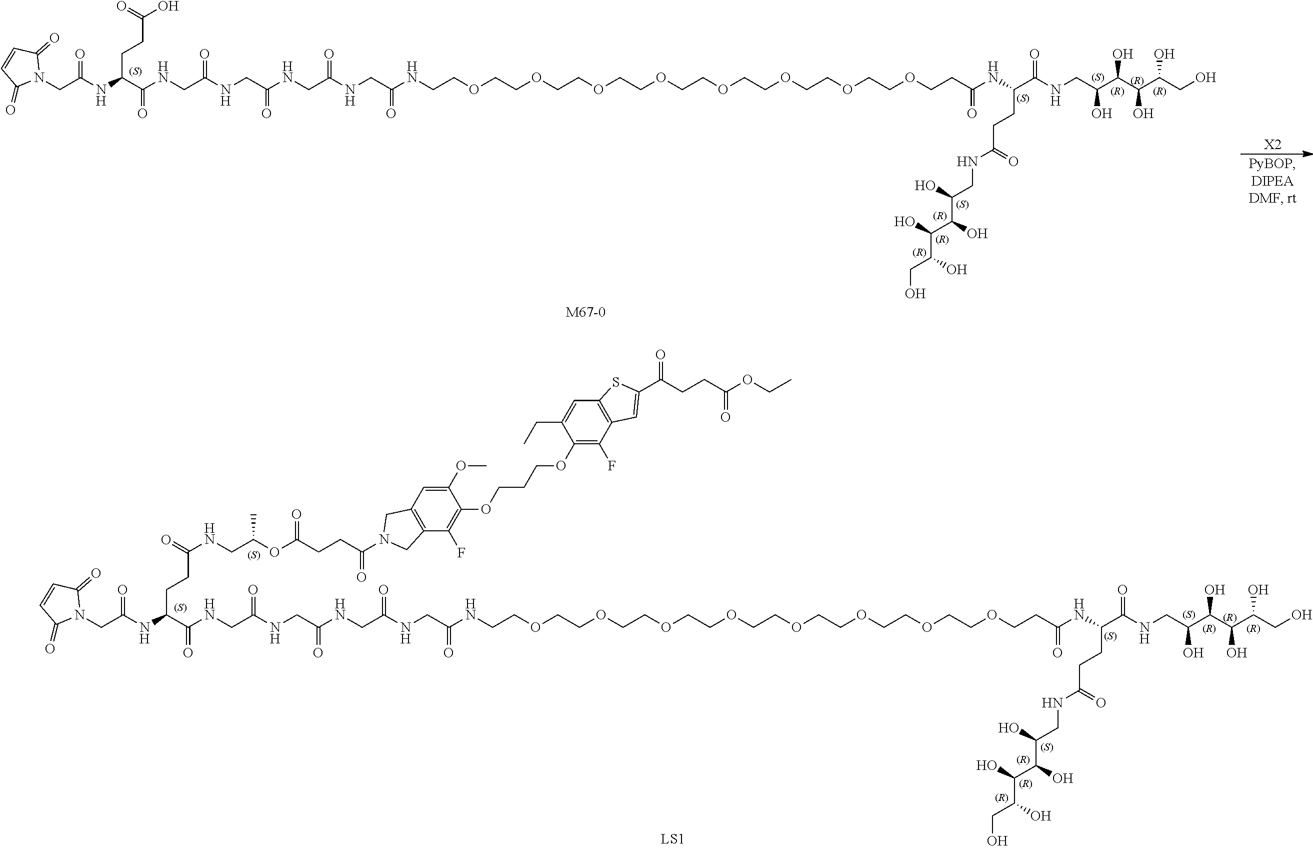

PyBOP (0.0870 g, 167.1819 mol) was added to a solution of (4S,47S,53S,54R,55R,56R)-4-(2-(2,5-dioxo-2,5-dihydro-1H-pyrrol-1-yl)acetamido)-53,54,55,56,57-pentahydroxy-5,8,11,14,17,45,50-heptaoxo-47-(((2S,3R,4R,5R)-2,3,4,5,6-pentahydroxyhexyl)carbamoyl)-21,24,27,30,33,36,39,42-octaoxa-6,9,12,15,18,46,51-heptaazaheptapentacontanoic acid (0.0701 g, 50.3817 mol), ethyl-4-[5-[3-[2-[4-[(1S)-2-amino-i-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (48.3000 mg, 64.9884 mol), and DIEA (40.8100 mg, 315.7632 mol) in DMF (3 mL) at 20° C. The reaction mixture was stirred for 2 h at 20° C. . The resulting reaction mixture was directly purified on C18 column ACN/0.1% TFA-H20(0-100%). The purified fraction was concentrated and lyophilized overnight. The residue was further purified by prep-HPLC (the mobile phase: ACN/0.1% TFA-H₂O(0-50%) ). The purified fraction was lyophilized overnight. There was ethyl-4-[5-[3-[2-[4-[(1S)-2-[[(4S)-4-[[2-(2,5-dioxopyrrol-1-yl)acetyl]amino]-5- oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]pentanoy 1]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.05020 g, 47.9010% yield) obtained as a white solid. LCMS: (ESI, m/z): [M+2H]2- =1041.000.

### Example IV-2 Synthesis of Linker-STING Agonist Compound LS2

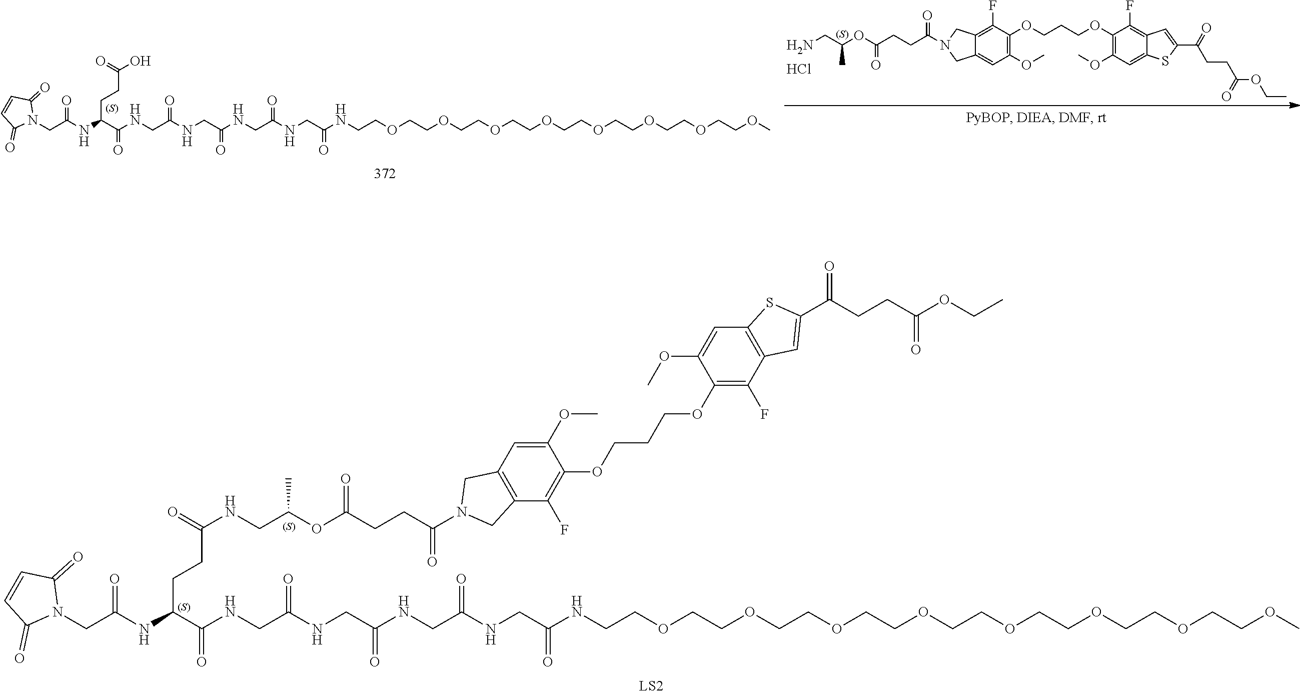

PyBOP (0.0709 g, 136.2436 mol) was added to a solution of (S)-40-(2-(2,5-dioxo-2,5-dihydro-1H-pyrrol-1-yl)acetamido)-27,30,33,36,39-pentaoxo-2,5,8,11,14,17,20,23-octaoxa-26,29,32,35,38-pentaazatritetracontan-43-oic acid (0.0422 g, 48.0699 mol), ethyl 4-[5-[3-[2-[4-[(1S)-2-amino-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.0415 g, 55.8389 mol, HCl salt), and DIEA (37.1000 mg, 287.0575 μmol) in DMF (4 mL) at 25° C. The reaction mixture was stirred for 2 h at 25° C. The resulting reaction mixture was directly purified on C18 column ACN/0.1% TFA-H₂O(0-100%). The purified fraction was lyophilized. The residue was further purified by prep-HPLC (the mobile phase: ACN/0.1% TFA-H₂O (0-50%)). The purified fraction was lyophilized overnight. There was ethyl 4-[5-[3-[2-[4-[(1S)-2-[[(4S)-4-[[2-(2,5-dioxopyrrol-1-yl)acetyl]amino]-5-[[2-[[2-[[2-[[2-[2-[2-[2-[2-[2-[2-[2-(2-methoxyethoxy)ethoxy]ethoxy]ethoxy]ethoxy]ethoxy]ethoxy]ethylamino]-2-oxo-ethyl]amino]-2-oxo-ethyl]amino]-2-oxo-ethyl]amino]-2-oxo-ethyl]amino]-5-oxo-pentanoyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.02126 g, 28.2310% yield) obtained as a white solid. LCMS: (ESI, m/z): [M+2H]⁺=784.250.

### Example IV-3 Synthesis of Linker-STING Agonist Compound LS3

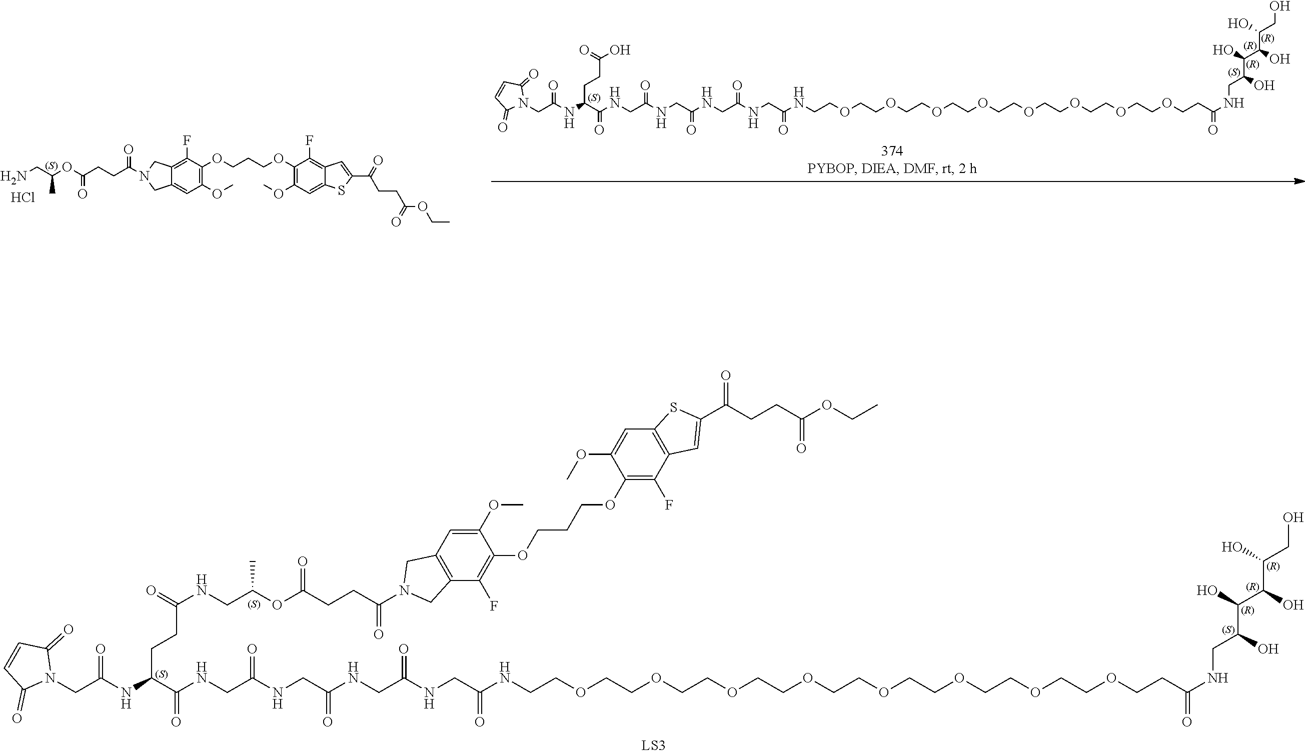

6,9,12,15,18,46-hexaazadopentacontanoic acid (39.1 mg, 35.5747 mol), and N,N-diisopropylethylamine (28.1960 mg, 218.1637 mol) in N,N-dimethylformamide (2 mL) at 25° C. The reaction mixture was stirred for 2 h at 25° C. The crude product was purified by Prep-HPLC with the following conditions: (CXTH LC6000, HPLC-P4): Column, XB—C18, 30*150 mm, 5 um; mobile phase, Water (0.1% TFA) and MeCN—20-46% B(2-32-36 min); Detector, uv 232 nm). The solvent was removed by lyophilization. There was ethyl 4-[5-[3-[2-[4-[(1S)-2-[[(4S)-4-[[2-(2,5-dioxopyrrol-1-yl)acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(2S,3R,4R,5R)-2,3,4,5,6-pentahydroxyhexyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]pentanoyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (16.3 mg, 9.1172 mol, 25.6283% yield) obtained as a white solid. LCMS: (ESI, m/z): [M+2H]⁺=893.91.

### Example IV-4 Synthesis of Linker-STING Agonist Compound LS4

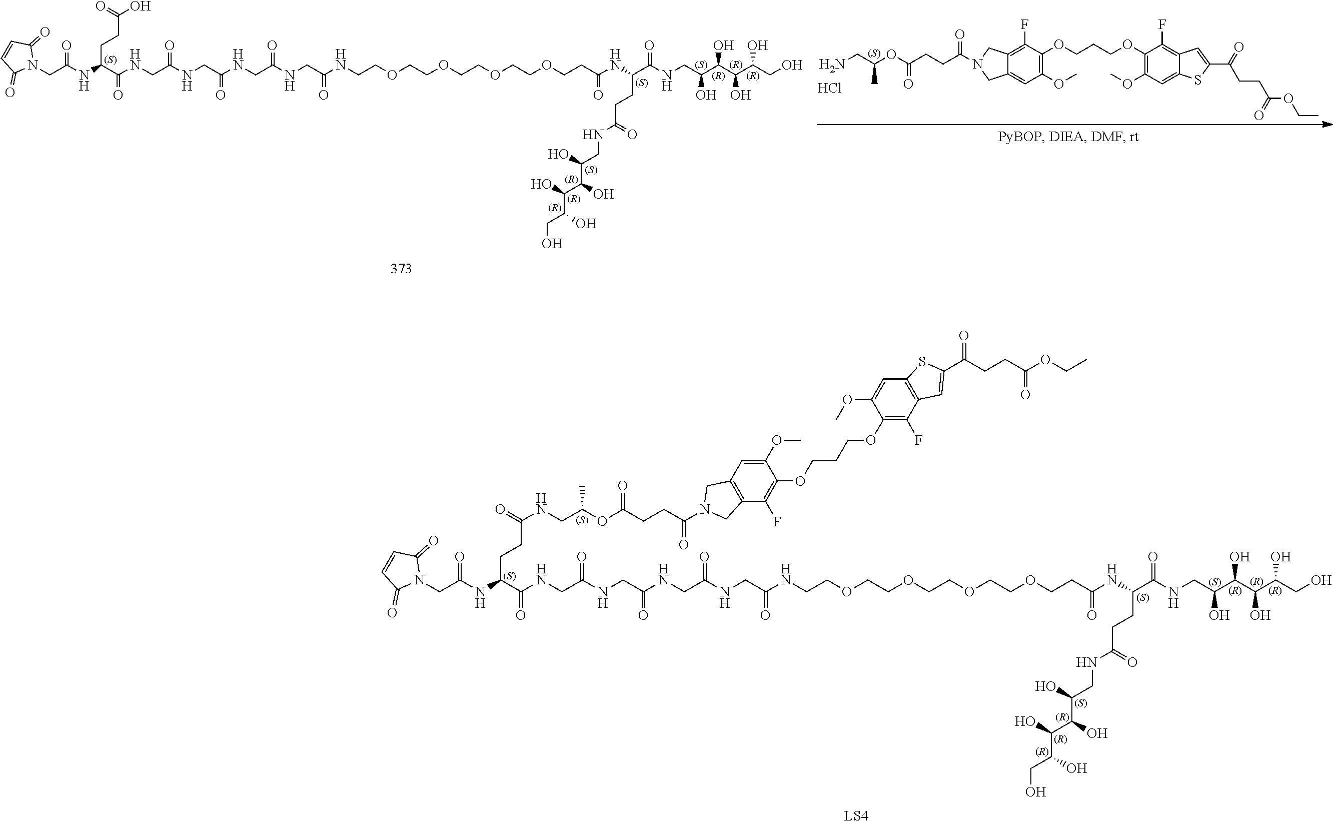

(S)-1-aminopropan-2-yl-4-(5-(3-((2-(4-ethoxy-4-oxobutanoyl)-4-fluoro-6- methoxybenzo[b]thiophen-5-yl)oxy)propoxy)-4-fluoro-6-methoxyisoindolin-2-yl)-4-oxobutanoate hydrochloride (0.0265 g, 35.6561 mol) was added to a solution of (4S,35S,41S,42R,43R,44R)—4-(2-(2,5-dioxo-2,5-dihydro-1H-pyrrol-1-yl)acetamido)-41,42,43,44,45-pentahydroxy-5,8,11,14,17,33,38-heptaoxo-35-(((2S,3R,4R,5R)-2,3,4,5,6-pentahydroxyhexyl)carbamoyl)-21,24,27,30-tetraoxa-6,9,12,15,18,34,39-heptaazapentatetracontanoic acid (0.0357 g, 29.3786 mol), PyBOP (0.0470 g, 90.3166 mol) and N,N-Diisopropylethylamine (0.0273 g, 211.2310 mol) in DMF (2 mL) at 20° C. under N₂ atmosphere. The reaction mixture was stirred for at 20° C. The reaction mixture was purified on C18 column ACN/H₂O (0.1% TFA)(0-40%). The purified fraction was dried by lyophilization. The residue was purified by Prep-HPLC. The purified fraction was dried by lyophilization. There was ethyl 4-[5-[3-[2-[4-[(1S)-2-[[(4S)-4-[[2-(2,5-dioxopyrrol-1-yl)acetyl]amino]-5-oxo-5-[[2-oxo-2-[[2-oxo-2-[[2-oxo-2-[[2-oxo-2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethylamino]ethyl]amino]ethyl]amino]ethyl]amino]ethyl]amino]pentanoyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.021 g, 11.0300 mol, 37.1211% yield) obtained as a white solid.

LCMS: (ESI, m/z): [M+2H]⁺=952.900.

### Example IV-5 Synthesis of Linker-STING Agonist Compound LS5

### Step a:

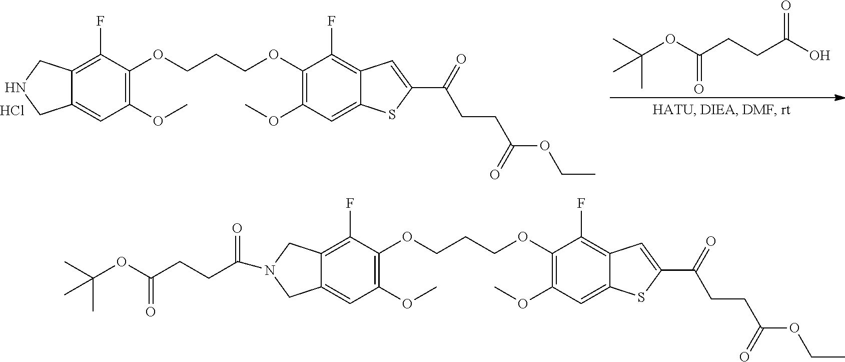

DIEA (1.6 mL, 9.1858 mmol) was added to a solution of ethyl 4-[4-fluoro-5-[3-(4-fluoro-6-methoxy-isoindolin-5-yl)oxypropoxy]-6-methoxy-benzothiophen-2-yl]-4-oxo-butanoate (0.85 g, 1.4504 mmol, HCl salt), 4-(tert-butoxy)-4-oxobutanoic acid (0.36 g, 2.0787 mmol), and HATU (0.88 g, 2.3144 mmol) in DMF (18 mL) at 18° C. The reaction mixture was stirred for 2 h at 18° C. The resulting reaction mixture was diluted with water (150 mL), and then extracted with EA(150 mL). The organic layer was separated and washed with brine (2×150 mL). The organic layer was collected and dried over Na₂SO₄, filtered and evaporated under reduced pressure. The residue was purified on C18 column ACN/0.1% TFA-H20(0-100%). The purified fraction was concentrated and lyophilized overnight. There was ethyl 4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.62 g, 60.5682% yield) obtained as a brown solid. LCMS: (ESI, m/z): [M+2H]⁺=707.300. ¹H NMR (400 MHz, DMSO-d₆) δ 8.31 (s, 1H), 7.56 (s, 1H), 6.89 (s, 0.5H), 6.86 (s, 0.5H), 4.77 (s, 2H), 4.55 (d, J=8.0 Hz, 2H), 4.25 (t, J=5.8 Hz, 2H), 4.20 (t, J=5.9 Hz, 2H), 4.06 (q, J=7.1 Hz, 2H), 3.88 (s, 3H), 3.77 (s, 3H), 3.39-3.35 (m, 2H), 2.66 (t, J=6.2 Hz, 2H), 2.59-2.52 (m, 2H), 2.49-2.42 (m, 2H), 2.09-2.00 (m, 2H), 1.39 (s, 9H), 1.18 (t, J=7.1 Hz, 3H).

### Step b:

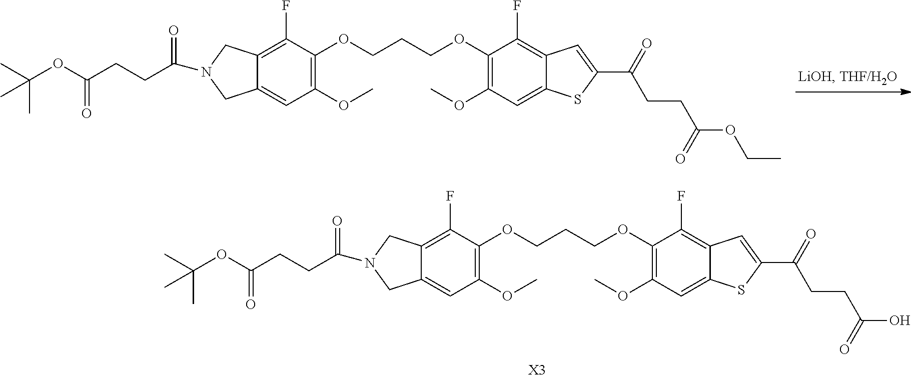

LiOH (0.043 g, 1.7955 mmol) was added to a solution of ethyl 4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.60 g, 850.1462 mol) in THF (10 mL) and H20 (5 mL) at 20° C. The reaction mixture was stirred for 2 h at 20° C. The resulting reaction mixture was adjusted with 6 N HCl solution to PH=6, and then evaporated under reduced pressure. The residue was purified on C18 column ACN/0.1% TFA-H20(0-100%). The purified fraction was concentrated and lyophilized overnight. There was 4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoic acid (0.43 g, 74.6332% yield) obtained as a slight yellow solid. LCMS: (ESI, m/z): [M+H]⁺=678.250. ¹H NMR (400 MHz, DMSO-d₆) δ 12.18 (s, 1H), 8.30 (s, 1H), 7.56 (s, 1H), 6.89 (s, 0.5H), 6.87 (s, 0.5H), 4.77 (s, 2H), 4.55 (d, J=6.8 Hz, 2H), 4.25 (t, J=5.5 Hz, 2H), 4.19 (t, J=5.9 Hz, 2H), 3.88 (s, 3H), 3.77 (s, 3H), 3.32 (t, J=6.2 Hz, 2H), 2.65-2.51 (m, 4H), 2.49-2.42 (m, 2H), 2.07-2.00 (m, 2H), 1.39 (s, 9H).

### Step c:

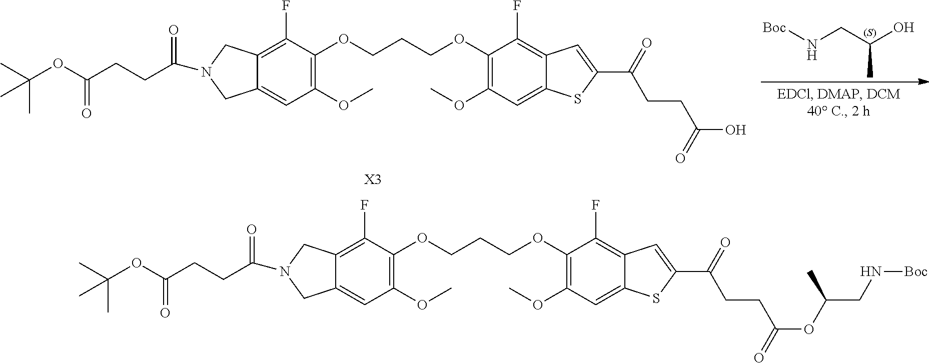

EDCI (0.29 g, 1.5128 mmol) was added to a solution of 4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoic acid (0.42 g, 619.7359 mol), tert-butyl (S)-(2-hydroxypropyl)carbamate (0.34 g, 1.9404 mmol), and DMAP (0.05 g, 409.2758 mol) in DCM (20 mL) at 20° C. The reaction mixture was heated to 35° C. and stirred for 2 h. The resulting reaction mixture was evaporated under reduced pressure. The residue was purified on C-18 column ACN/0.1% TFA-H₂O(0-100%). The purified fraction was concentrated and lyophilized overnight. There was [(1S)-2-(tert-butoxycarbonylamino)-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.42 g, 81.1707% yield) obtained as a yellowish solid. LCMS: (ESI, m/z): [M+H]⁺=835.350.

### Step d:

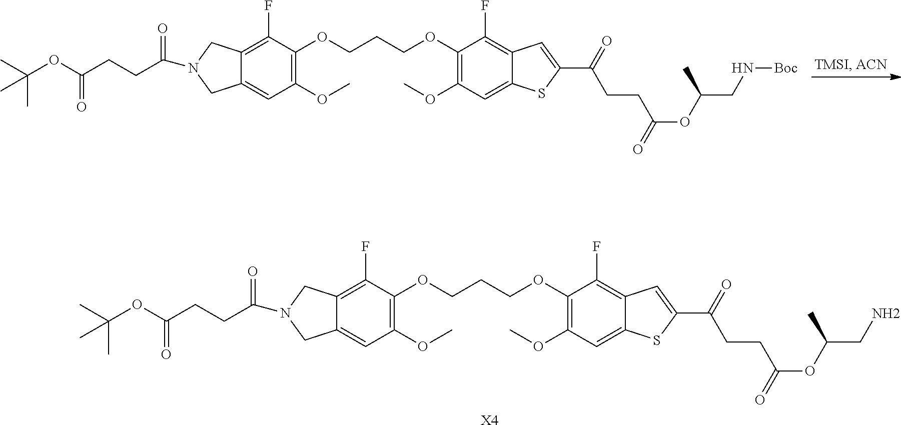 [SMILES: COc1cc2c(c(F)c1OCCCOc1c(OC)cc3sc(C(=O)CCC(=O)O[C@@H](C)CNC(=O)OC(C)(C)C)cc3c1F)CN(C(=O)CCC(=O)OC(C)(C)C)C2 ; COc1cc2c(c(F)c1OCCCOc1c(OC)cc3sc(C(=O)CCC(=O)O[C@@H](C)CN)cc3c1F)CN(C(=O)CCC(=O)OC(C)(C)C)C2 ; "X4"]

Iodotrimethylsilane (0.0354 g, 176.9177 mol) was added to a solution of [(1S)-2-(tert-butoxycarbonylamino)-1-methyl-ethyl]-4-[5-[3-[2-(4-tert-butoxy-4-oxo-butanoyl)-4-fluoro-6- methoxy-isoindolin-5-yl]oxypropoxy]-4-fluoro-6-methoxy-benzothiophen-2-yl]-4-oxo-butanoate (0.1187 g, 142.1697 μmol) in ACN (6 mL) at 0° C. The reaction mixture was stirred for 0.5 h at 0° C., and then warmed to room temperature and continually stirred for 2 h. The reaction mixture was quenched with H20 (4 mL). The resulting reaction mixture was purified on C-18 column ACN/0.1% TFA-H₂O(0-100%). The purified fraction was lyophilized overnight. There was [(1S)-2-amino-1-methyl-ethyl]4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.0699 g, 66.9113% yield) obtained as a yellowish solid. LCMS: (ESI, m/z): [M+H]⁺=735.250. ¹H NMR (400 MHz, DMSO-d₆) δ 8.31 (s, 1H), 7.88 (s, 3H), 7.56 (s, 1H), 6.89 (s, 0.5H), 6.88 (s, 0.5H), 5.05-4.92 (m, 1H), 4.78 (s, 2H), 4.56 (d, J=7.4 Hz, 2H), 4.25 (t, J=5.7 Hz, 2H), 4.19 (t, J=5.8 Hz, 2H), 3.88 (s, 3H), 3.77 (s, 3H), 3.39 (t, J=6.2 Hz, 2H), 3.07-2.94 (m, 2H), 2.78-2.69 (m, 2H), 2.58-2.54 (m, 2H), 2.48-2.42 (m, 2H), 2.09-2.01 μm , 2H), 1.39 (s, 9H), 1.21 (d, J=6.4 Hz, 3H).

### Step e:

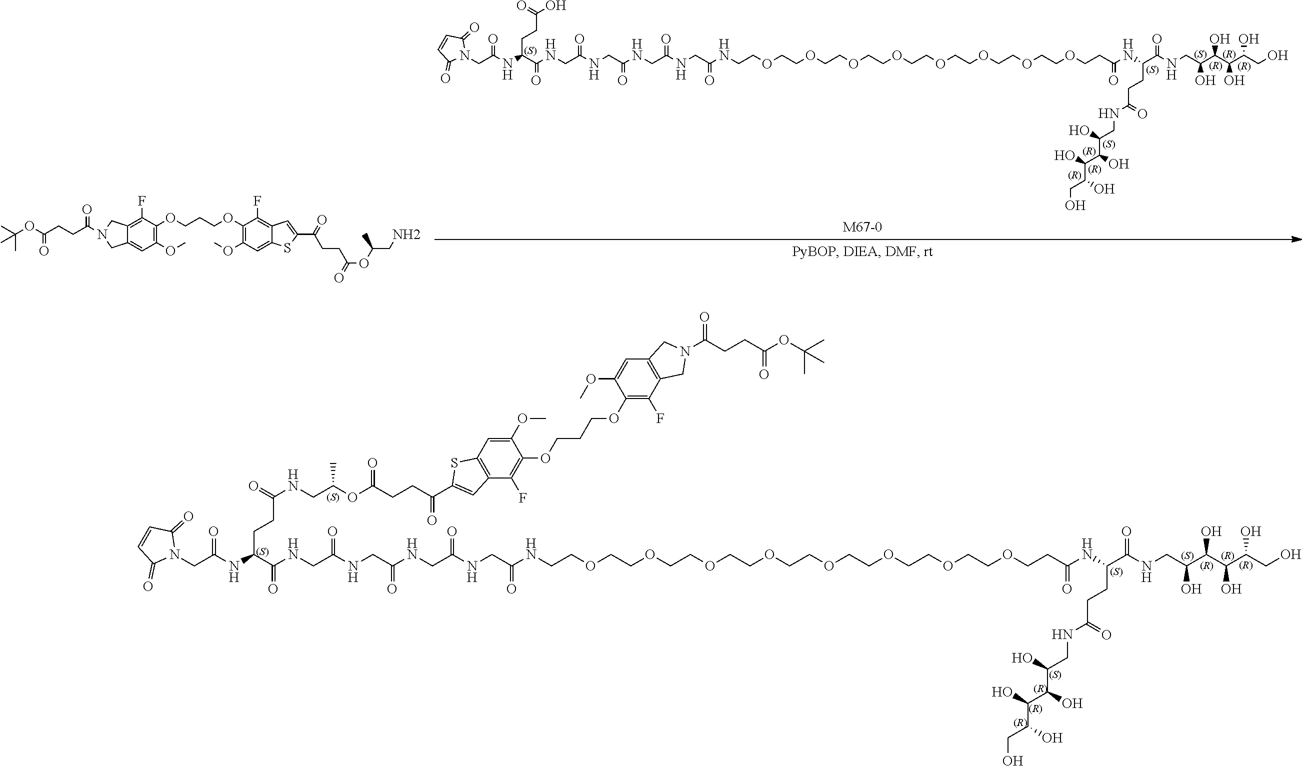

PyBOP (0.0525 g, 100.8856 mol) was added to a solution of [(1S)-2-amino-1-methyl-ethyl]-4-[5-[3-[2-(4-tert-butoxy-4-oxo-butanoyl)-4-fluoro-6-methoxy-isoindolin-5- yl]oxypropoxy]-4-fluoro-6-methoxy-benzothiophen-2-yl]-4-oxo-butanoate (30.1000 mg, 40.9634 mol), (4S,47S,53S,54R,55R,56R)-4-(2-(2,5-dioxo-2,5-dihydro-1H-pyrrol-1-yl)acetamido)-53,54,55,56,57-pentahydroxy-5,8,11,14,17,45,50-heptaoxo-47-(((2S,3R,4R,5R)-2,3,4,5,6-pentahydroxyhexyl)carbamoyl)-21,24,27,30,33,36,39,42-octaoxa-6,9,12,15,18,46,51-heptaazaheptapentacontanoic acid (0.0456 g, 32.7732 mol), and DIEA (0.045 mL, 258.3517 mol) in DMF (3.0 mL) at 21° C. The reaction mixture was stirred for 2 h at 21° C. The resulting reaction mixture was directly purified on C18 column ACN/0.1% TFA-H₂O(0-100%). The purified fraction was lyophilized overnight. There was [(1S)-2-[[(4S)-4-[[2-(2,5-dioxopyrrol-1-yl)acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4- oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]pentanoyl]amino]-1-methyl-ethyl]4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.0800 g, 92.6380% yield) obtained as a white solid.

LCMS: (ESI, m/z): [M+2H]²=1054.950.

### Step f:

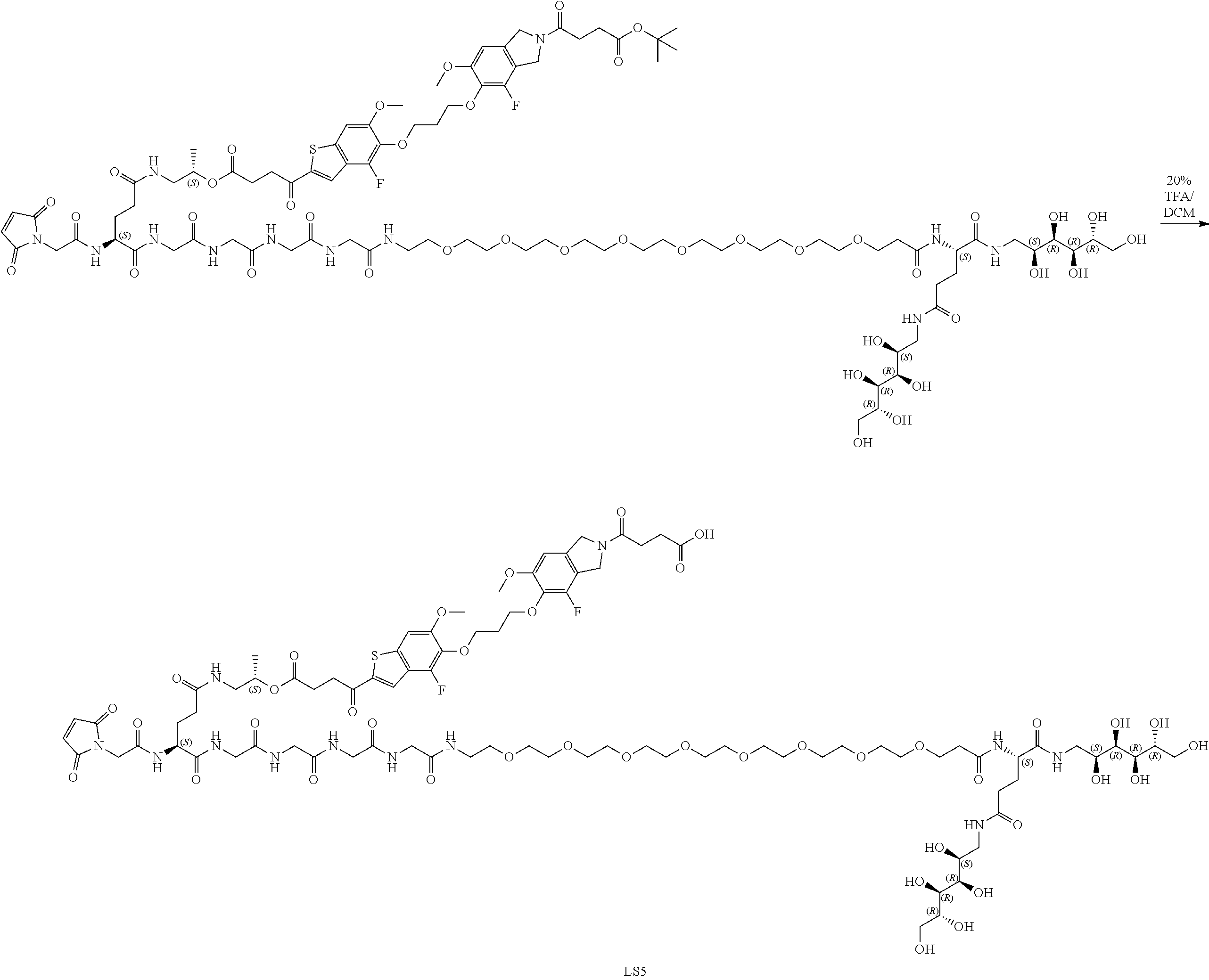

[(1S)-2-[[(4S)-4-[[2-(2,5-dioxopyrrol-1-yl)acetyl]amino]-5-oxo-5-[[2-oxo-2-[[2-oxo-2-[[2-oxo-2-[[2- oxo-2-[2-[2-[2-[2-[2-[2-[2-[2-[3-oxo-3-[[(1S)-4-oxo-4-[[(2S,3R,4R,5R)-2,3,4,5,6- pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]pentanoyl]amino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.0985 g, 46.7231 mol) was dissolved in 20% TFA (8 mL) in DCM at 0° C. The reaction mixture was stirred for 1 h at 22° C. The reaction mixture was concentrated under reduced pressure. The resulting residue was lyophilized overnight. The crude was further purified by prep-HPLC (the mobile phase: ACN/0.1% TFA-H₂O(0-50%)). The purified fraction was lyophilized overnight. There was 4-[5-[3-[2-[4-[(1S)-2-[[(4S)-4-[[2-(2,5-dioxopyrrol-1-yl) acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]a mino]pentanoyl]amino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (96.1000 mg, 100.2310% yield) obtained as a white solid. LCMS: (ESI, m/z): [M+2H]²⁺=1026.850

### Example IV-6 Synthesis of Linker-STING Agonist Compound LS6

### Step a:

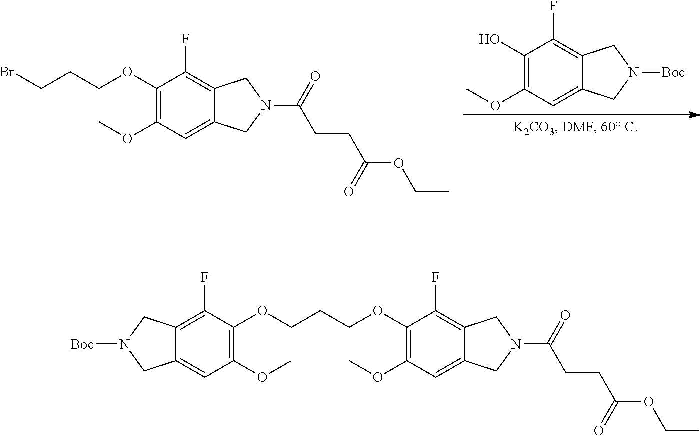 [SMILES: CCOC(=O)CCC(=O)N1Cc2cc(OC)c(OCCCBr)c(F)c2C1 ; COc1cc2c(c(F)c1O)CN(C(=O)OC(C)(C)C)C2 ; CCOC(=O)CCC(=O)N1Cc2cc(OC)c(OCCCOc3c(OC)cc4c(c3F)CN(C(=O)OC(C)(C)C)C4)c(F)c2C1]

K₂CO₃ (1.19 g, 8.6104 mmol) was added to a solution of ethyl 4-(5-(3-bromopropoxy)-4-fluoro-6-methoxyisoindolin-2-yl)-4-oxobutanoate (1.19 g, 2.7528 mmol) and tert-butyl 4-fluoro-5-hydroxy-6-methoxyisoindoline-2-carboxylate (0.91 g, 3.2122 mmol) in DMF (25 mL) at 18° C. The reaction mixture was heated to 60° C. and stirred overnight. The resulting reaction mixture was diluted with H20 (200 mL), and then extracted with EA (200 mL). The organic layer was separated and washed with brine (200×2 mL). The organic layer was collected and dried over Na₂SO₄, filtered and evaporated under reduced pressure. The residue was purified by silica gel column chromatography, eluting with ethyl acetate/n-hexane (0-100%). The purified fraction was concentrated and dried under reduced pressure. There was tert-butyl 5-[3-[2-(4-ethoxy-4-oxo-butanoyl)-4-fluoro-6- methoxy-isoindolin-5-yl]oxypropoxy]-4-fluoro-6-methoxy-isoindoline-2-carboxylate (1.49 g, 85.2827% yield) obtained as a slight red solid. LCMS: (ESI, m/z): [M+H-Boc]*=535.250.

¹H NMR (400 MHz, Chloroform-d) δ 6.62 (s, 1H), 6.59 (s, 1H), 4.83 (d, J=7.8 Hz, 2H), 4.77 (d, J=6.5 Hz, 2H), 4.64 (s, 4H), 4.33-4.23 (m, 4H), 4.17 (q, J=7.3 Hz, 2H), 3.85 (s, 6H), 2.81-2.62 (m, 4H), 2.24-2.15 (m, 2H), 1.52 (s, 9H), 1.28 (t, J=6.8 Hz, 3H).

### Step b:

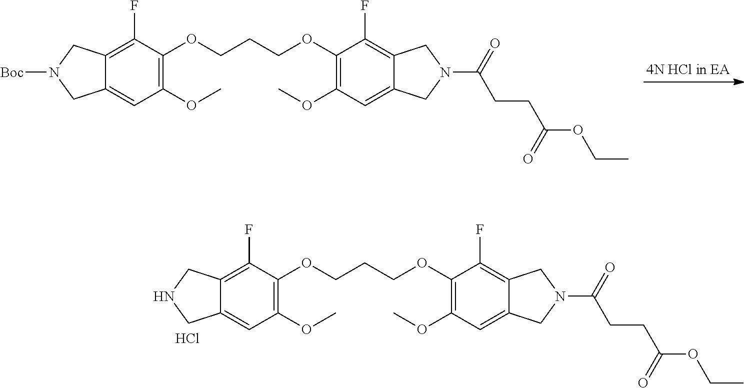 [SMILES: CCOC(=O)CCC(=O)N1Cc2cc(OC)c(OCCCOc3c(OC)cc4c(c3F)CN(C(=O)OC(C)(C)C)C4)c(F)c2C1 ; CCOC(=O)CCC(=O)N1Cc2cc(OC)c(OCCCOc3c(OC)cc4c(c3F)CNC4)c(F)c2C1 ; Cl]

4 N HCl solution (15 mL, 60 mmol) in EA was added to a solution of tert-butyl 5-[3-[2-(4-ethoxy-4-oxo-butanoyl)-4-fluoro-6-methoxy-isoindolin-5-yl]oxypropoxy]-4-fluoro-6-methoxy-isoindoline-2-carboxylate (0.352 g, 554.6251 mol) in EA (5 mL) at 19° C. The reaction mixture was stirred for 2 h at 19° C. The resulting reaction mixture was concentrated under reduced pressure. There was ethyl 4-[4-fluoro-5-[3-(4-fluoro-6-methoxy-isoindolin-5-yl)oxypropoxy]-6-methoxy-isoindolin-2-yl]-4-oxo-butanoate (0.27 g, 85.2553% yield, HCl salt) obtained as a red solid. LCMS: (ESI, m/z): [M+H]⁺=535.250. ¹H NMR (400 MHz, DMSO-d₆) δ 10.10 (s, 2H), 6.98 (s, 1H), 6.94 (s, 0.5H), 6.91 (s, 0.5H), 4.83 (d, J=8.4 Hz, 2H), 4.59 (s, 2H), 4.50 (s, 2H), 4.46 (s, 2H), 4.16 (q, J=6.2 Hz, 4H), 4.05 (q, J=7.1 Hz, 2H), 3.85-3.74 (m, 6H), 2.67-2.60 (m, 2H), 2.58-2.52 (m, 2H), 2.06-1.96 (m, 2H), 1.18 (t, J=7.1 Hz, 3H).

### Step c:

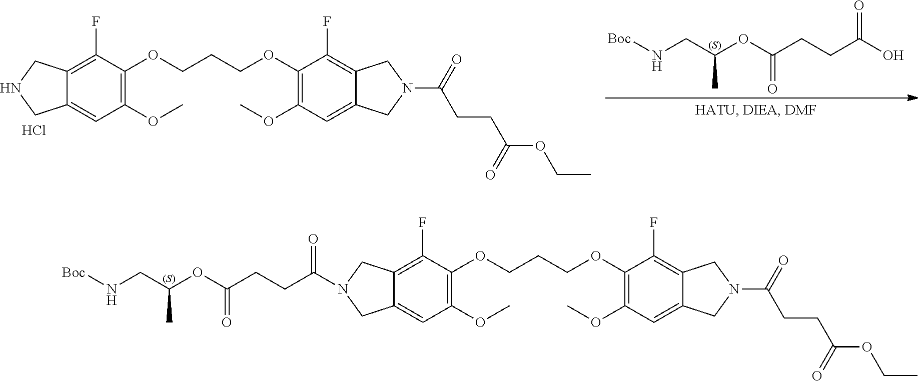

HATU (0.367 g, 965.2070 mol) was added to a solution of (S)-4-((1-((tert-butoxycarbonyl)amino)propan-2-yl)oxy)-4-oxobutanoic acid (0.216 g, 1.0323 mmol) and DIEA (363.5800 mg, 2.8132 mmol) in DMF (8 mL) at 18° C. After stirring for 0.5 h, ethyl 4-[4-fluoro-5-[3-(4-fluoro-6-methoxy-isoindolin-5-yl)oxypropoxy]-6-methoxy-isoindolin-2-yl]-4-oxo-butanoate (0.258 g, 451.8320 mol, HCl salt) in DMF (5 mL) was added. The reaction mixture was stirred for 2 h at 25° C. The resulting reaction mixture was diluted with brine (60 mL), and then extracted with EA (60 mL). The organic layer was separated and washed with brine (2×60 mL). The organic layer was collected and dried over Na₂SO₄, filtered and evaporated under reduced pressure. The residue was purified by silica gel column chromatography, eluting with MeOH/DCM (0-5%). The purified fraction was concentrated and dried under reduced pressure. There was ethyl 4-[5-[3-[2-[4-[(1S)-2-(tert-butoxycarbonylamino)-1-methyl- ethoxy]-4-oxo-butanoyl]-4-fluoro-6-methoxy-isoindolin-5-yl]oxypropoxy]-4-fluoro-6-methoxy-isoindolin-2-yl]-4-oxo-butanoate (0.219 g, 61.2118% yield) obtained as a brown semi-solid. LCMS: (ESI, m/z): [M+H-Boc]⁺=692.350.

### Step d:

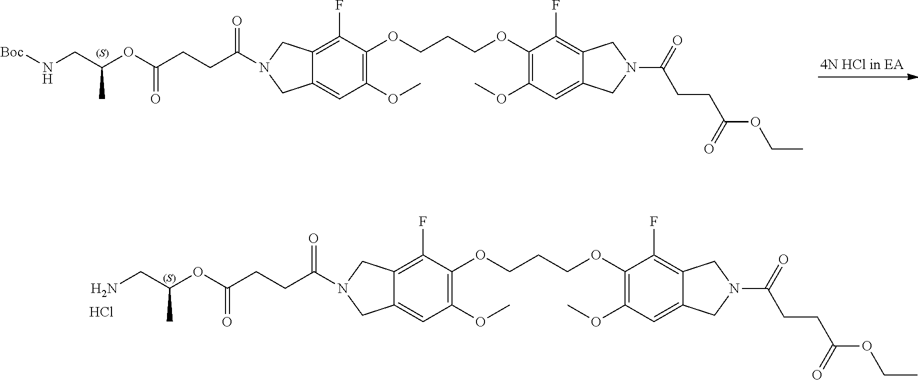

4 N HCl solution (8 mL, 32 mmol) in EA was added to a solution of ethyl 4-[5-[3-[2-[4-[(1S)-2-(tert-butoxycarbonylamino)-1-methyl-ethoxy]-4-oxo-butanoyl]-4-fluoro-6-methoxy-isoindolin-5-yl]oxypropoxy]-4-fluoro-6-methoxy-isoindolin-2-yl]-4-oxo-butanoate (0.208 g, 262.6826 mol) in EA (2 mL) at 17° C. The reaction mixture was stirred for 2 h at 17° C. The reaction mixture was concentrated under reduced pressure. The resulting residue was lyophilized overnight. There was ethyl 4-[5-[3-[2-[4-[(1S)-2-amino-1-methyl-ethoxy]-4-oxo-butanoyl]-4-fluoro-6-methoxy-isoindolin-5-yl]oxypropoxy]-4-fluoro-6-methoxy-isoindolin-2-yl]-4-oxo-butanoate (0.166 g, 91.3587% yield, HCl salt) obtained as a brown solid. LCMS: (ESI, m/z): [M+H]⁺=692.350.

### Step e:

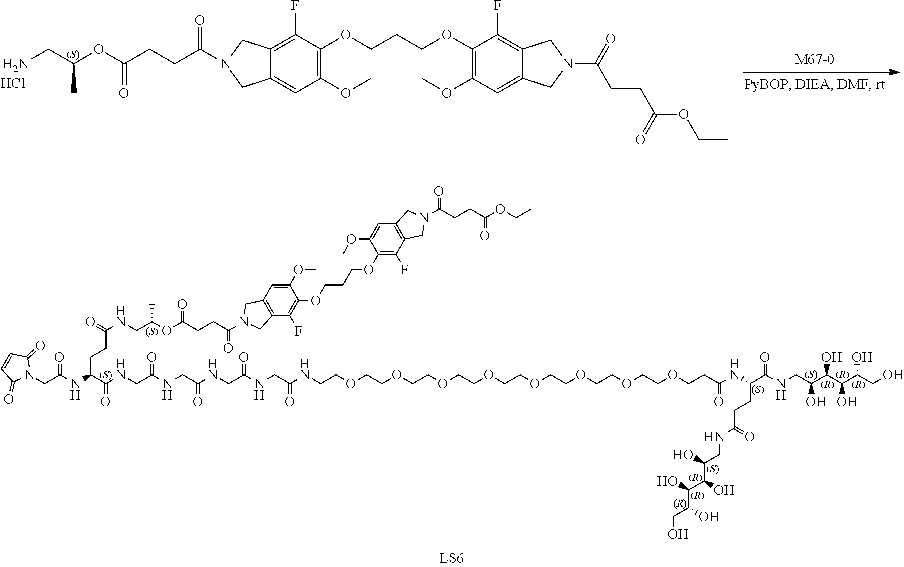

PyBOP (0.0591 g, 113.5684 mol) was added to a solution of ethyl 4-[5-[3-[2-[4-[(1S)-2-amino-1-methyl-ethoxy]-4-oxo-butanoyl]-4-fluoro-6-methoxy-isoindolin-5-yl]oxypropoxy]-4-fluoro-6-methoxy-isoindolin-2-yl]-4-oxo-butanoate (0.0364 g, 52.6229 mol, HCl salt), (4S,47S,53S,54R,55R,56R)-4-(2-(2,5-dioxo-2,5-dihydro-1H-pyrrol-1-yl)acetamido)-53,54,55,56,57-pentahydroxy-5,8,11,14,17,45,50-heptaoxo-47-(((2S,3R,4R,5R)-2,3,4,5,6-pentahydroxyhexyl)carbamoyl)-21,24,27,30,33,36,39,42-octaoxa-6,9,12,15,18,46,51-heptaazaheptapentacontanoic acid (0.0544 g, 39.0979 mol), and DIEA (0.041 mL, 235.3871 mol) in DMF (3 mL) at 21° C. The reaction mixture was stirred for 4 h at 21° C. The resulting reaction mixture was directly purified on C18 column ACN/0.1% TFA-H₂O(0-100%). The purified fraction was lyophilized overnight. The residue was further purified by prep-HPLC (the mobile phase: ACN/0.1% TFA-H₂O(0-50%) ). The purified fraction was lyophilized overnight. There was ethyl 4-[5-[3-[2-[4-[(1S)-2-[[(4S)-4-[[2-(2,5-dioxopyrrol-1-yl)acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]pentanoyl]amino]-1-methyl-ethoxy]-4-oxo-butanoyl]-4-fluoro-6-methoxy-isoindolin-5-yl]oxypropoxy]-4-fluoro-6-methoxy-isoindolin-2-yl]-4-oxo-butanoate (21.8 mg, 27.0002% yield) obtained as a white solid. LCMS: (ESI, m/z): [M+2H]²⁺=1033.450.

### Example IV-7 Synthesis of Linker-STING Agonist Compound LS7

### Step a:

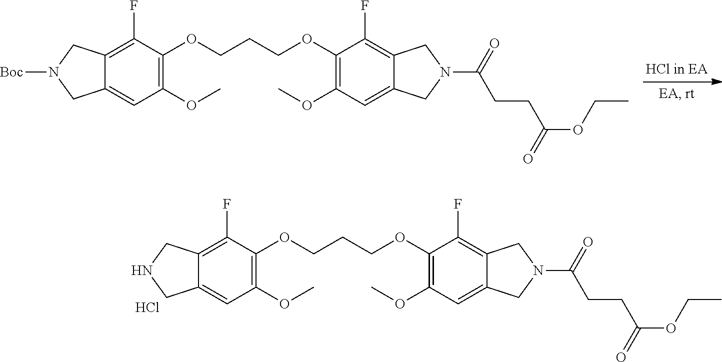 [SMILES: CCOC(=O)CCC(=O)N1Cc2cc(OC)c(OCCCOc3c(OC)cc4c(c3F)CN(C(=O)OC(C)(C)C)C4)c(F)c2C1 ; CCOC(=O)CCC(=O)N1Cc2cc(OC)c(OCCCOc3c(OC)cc4c(c3F)CNC4)c(F)c2C1 ; Cl]

HCl in EA (10 mL, 40 mmol) was added to a solution of tert-butyl 5-(3-((2-(4-ethoxy-4-oxobutanoyl)-4-fluoro-6-methoxyisoindolin-5-yl)oxy)propoxy)-4-fluoro-6-methoxyisoindoline-2-carboxylate (0.63 g, 992.6525 mol) in EA (5 mL) at 20° C. The reaction mixture was stirred for 2 h at 20° C. The resulting reaction mixture was concentrated under reduced pressure. There was ethyl 4-[4-fluoro-5-[3-(4-fluoro-6-methoxy-isoindolin-5-yl)oxypropoxy]-6-methoxy-isoindolin-2-yl]-4-oxo-butanoate (0.59 g, 1.1037 mmol, 111.1907% yield) obtained as a red solid, It is a crude product which was used directly to the next step. LCMS: (ESI, m/z): [M+H]⁺=535.220. H NMR (400 MHz, DMSO-d₆) δ 10.10 (s, 2H), 6.98 (s, 1H), 6.94 (s, 0.5H), 6.91 (s, 0.5H), 4.84 (s, 1H), 4.82 (s, 1H), 4.59 (s, 2H), 4.50 (s, 2H), 4.46 (s, 2H), 4.21-4.10 (m, 4H), 4.05 (q, J=7.1 Hz, 2H), 3.85-3.72 (m, 6H), 2.71-2.59 (m, 2H), 2.59-2.52 (m, 2H), 2.05-1.95 (m, 2H), 1.18 (t, J=7.1 Hz, 3H).

### Step b:

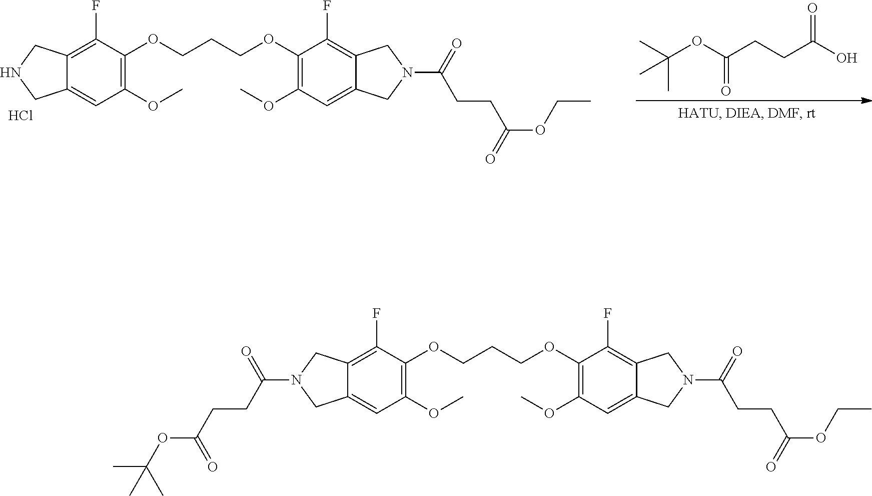

Ethyl-4-(4-fluoro-5-(3-((4-fluoro-6-methoxyisoindolin-5-yl)oxy)propoxy)-6-methoxyisoindolin-2-yl)-4-oxobutanoate hydrochloride (0.569 g, 996.4822 mol) was added to a solution of 4-(tert-butoxy)-4-oxobutanoic acid (0.370 g, 2.1364 mmol), HATU (0.628 g, 1.6516 mmol) and N,N-Diisopropylethylamine (0.625 g, 4.8359 mmol) in DMF (20 mL) at 20° C. under N₂ atmosphere. The reaction mixture was stirred for 3 h at 20° C. The reaction mixture was quenched with adding of EA(50 mL) at 20° C., washed with water (3×50 mL) and brine (50 mL). The organics dried over Na₂SO₄, filtered and evaporated under reduced pressure.

The residue was purified on C18 column ACN/H₂O (0.1% TFA)(0-75%). The purified fraction was concentrated and dried under vacuo. There was ethyl 4-[5-[3-[2-(4-tert-butoxy-4-oxo-butanoyl)-4-chloro-6-methoxy-isoindolin-5-yl]oxypropoxy]-4-fluoro-6-methoxy-benzothiophen-2-yl]-4-oxo-butanoate (0.48 g, 664.6216 mol, 67.2988% yield) obtained as a yellow oil. LCMS: (ESI, m/z): [M+H]⁺=691.300.

### Step c:

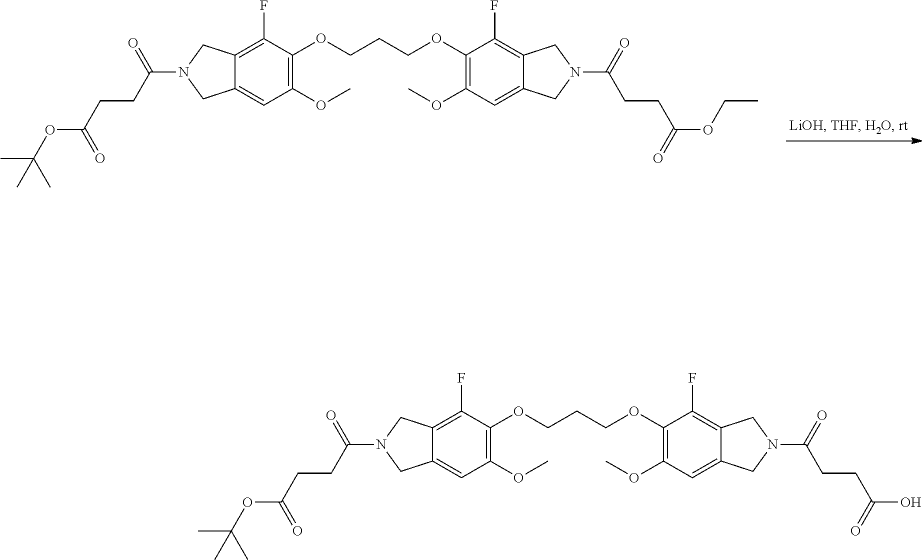

LiOH (0.043 g, 1.7955 mmol) was added to a solution of ethyl 4-[5-[3-[2-(4-tert-butoxy-4-oxo-butanoyl)-4-chloro-6-methoxy-isoindolin-5-yl]oxypropoxy]-4-fluoro-6-methoxy-benzothiophen-2-yl]-4-oxo-butanoate (0.520 g, 752.8308 mol) in Water (3 mL) and THF (6 mL) at 20° C. under open-air atmosphere. The reaction mixture was stirred for 2 h at 20° C. The reaction mixture was adjusted to pH=6 with HCl (1 mol/L). The mixture was purified on C18 column ACN/H₂O (0.1% TFA)(0-55%). The purified fraction was concentrated and dried under vacuo. There was 4-[5-[3-[2-(4-tert-butoxy-4-oxo-butanoyl)-4-fluoro-6-methoxy-isoindolin-5-yl]oxypropoxy]-4-fluoro-6-methoxy-isoindolin-2-yl]-4-oxo-butanoic acid (0.41 g, 618.7061 mol, 82.1840% yield) obtained as a white solid. LCMS: (ESI, m/z): [M+H]⁺=663.270.

### Step d:

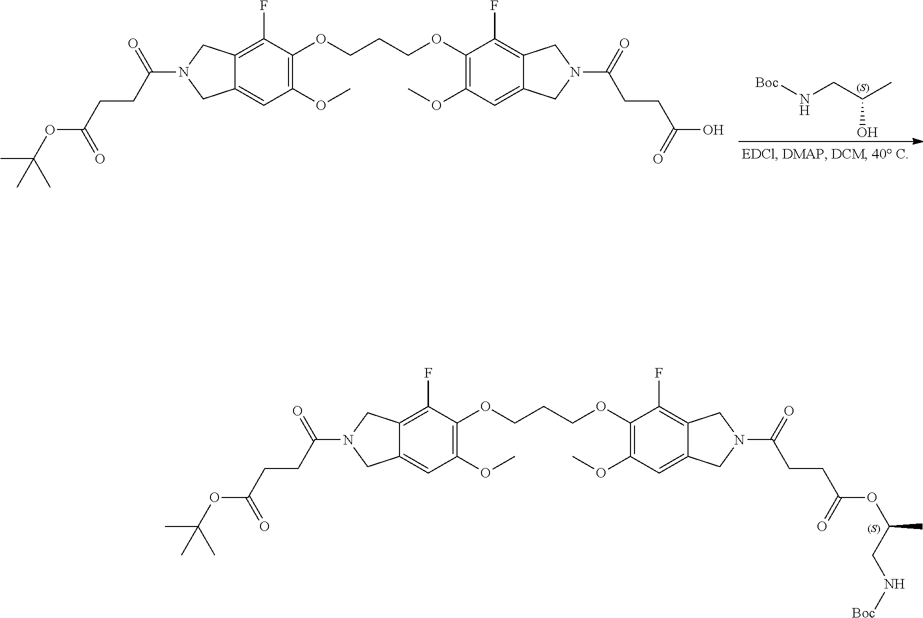

Tert-butyl (S)-(2-hydroxypropyl)carbamate (0.59 g, 3.3671 mmol) was added to a solution of EDCI (0.26 g, 1.3563 mmol), N-(4-pyridyl)dimethylamine (0.19 g, 1.5552 mmol) and 4-(5-(3-((2-(4-(tert-butoxy)-4-oxobutanoyl)-4-fluoro-6-methoxyisoindolin-5-yl)oxy)propoxy)-4-fluoro-6-methoxyisoindolin-2-yl)-4-oxobutanoic acid (0.41 g, 618.7064 mol) in DCM (20 mL) at 20° C. under open-air atmosphere. The reaction mixture was heated to 40° C. and stirred for 5 h. The reaction mixture was evaporated under reduced pressure. The residue was purified on C18 column ACN/H₂O (0.1% TFA)(0-70%). The purified fraction was concentrated and dried under vacuo. There was [(1S)-2-(tert-butoxycarbonylamino)-1-methyl-ethyl]4-[5-[3-[2-(4-tert-butoxy-4-oxo-butanoyl)-4-fluoro-6-methoxy-isoindolin-5-yl]oxypropoxy]-4-fluoro-6-methoxy-isoindolin-2-yl]-4-oxo-butanoate (0.25 g, 304.9216 mol, 49.2837% yield) obtained as a yellow solid. LCMS: (ESI, m/z): [M+H]⁺=820.380.

### Step e:

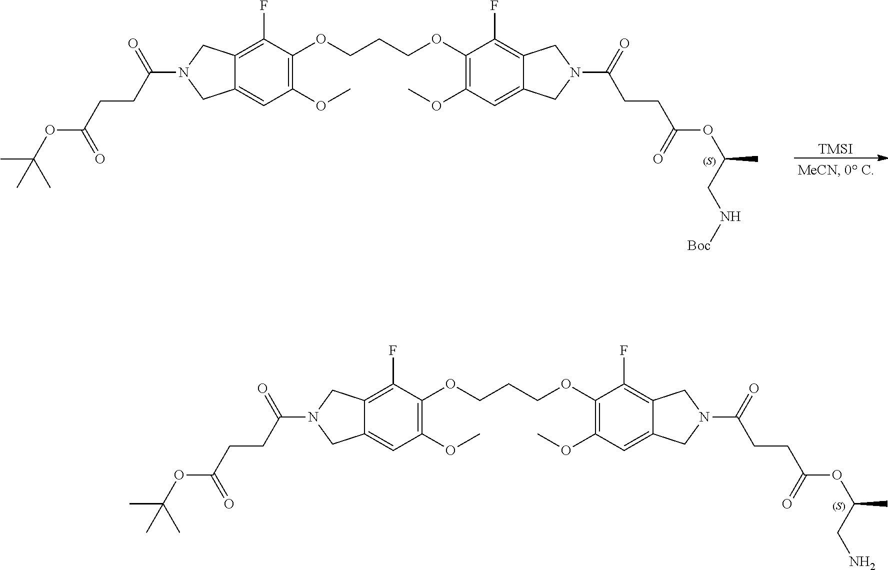

TMSI (0.104 g, 519.7583 mol) was added to a solution of [(1S)-2-(tert-butoxycarbonylamino)-1-methyl-ethyl]-4-[5-[3-[2-(4-tert-butoxy-4-oxo-butanoyl)-4-fluoro-6- methoxy-isoindolin-5-yl]oxypropoxy]-4-fluoro-6-methoxy-isoindolin-2-yl]-4-oxo-butanoate (0.212 g, 258.5735 mol) in MeCN (20 mL) at 0° C. under N₂ atmosphere. The reaction mixture was stirred for 0.5 h and warming up to 20° C. naturally. The reaction mixture was quenched with adding of water (10 mL), The mixture was purified on C18 column ACN/H₂O (0.1% TFA) (0-50%). The purified fraction was concentrated and dried by lyophilization. There was [(1S)-2-amino-i-methyl-ethyl]-4-[5-[3-[2-(4-tert-butoxy-4-oxo-butanoyl)-4-fluoro-6-methoxy-isoindolin -5-yl]oxypropoxy]-4-fluoro-6-methoxy-isoindolin-2-yl]-4-oxo-butanoate (0.12 g, 166.7205 mol, 64.4770% yield) obtained as a yellow solid. LCMS: (ESI, m/z): [M+H]⁺=720.320.

### Step f:

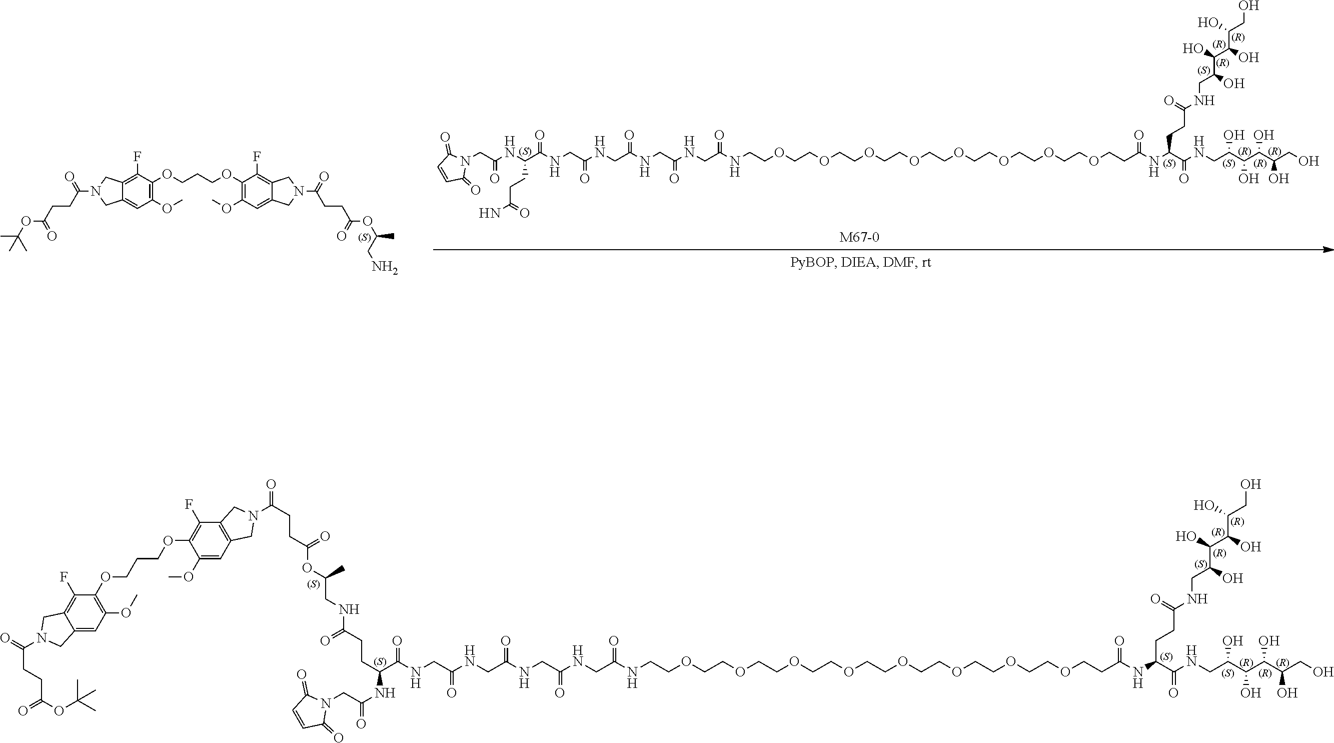

[(1S)-2-amino-1-methyl-ethyl]-4-[5-[3-[2-(4-tert-butoxy-4-oxo-butanoyl)-4-fluoro-6- methoxy-isoindolin-5-yl]oxypropoxy]-4-fluoro-6-methoxy-isoindolin-2-yl]-4-oxo-butanoate (0.0431 g, 59.8805 mol) and N,N-diisopropylethylamine (0.0554 g, 428.6519 mol) were added to a solution of tert-butyl-4-(5-(3-((2-((6S,12S,55S,61S,62R,63R,64R)-12-(2-(2,5-dioxo-2,5-dihydro-1H-pyrrol-1-yl)acetamido)-61,62,63,64,65-pentahydroxy-6-methyl-4,9,13,16,19,22,25,53,58-nonaoxo-55-(((2S,3R,4R,5R)-2,3,4,5,6-pentahydroxyhexyl) carbamoyl)-5,29,32,35,38,41,44,47,50-nonaoxa-8,14,17,20,23,26,54,59-octaazapentahexacontanoyl)-4-fluoro-6-methoxyisoindolin-5-yl)oxy)propoxy)-4-fluoro-6-methoxyisoindolin-2-yl)-4-oxobutanoate (0.0732 g, 52.6097 mol) and PyBOP (0.0751 g, 144.3145 mol) in DMF (2 mL) at 20° C. under open-air atmosphere. The reaction mixture was stirred for 2 h at 20° C. The mixture was purified on C18 column ACN/H₂O (0.1% TFA)(0-35%). The purified fraction was concentrated and dried by lyophilization. There was [(1S)-2-[[(4S)-4-[[2-(2,5-dioxopyrrol-1-yl)acetyl]amino]-5-oxo-5-[[2-oxo-2-[[2-oxo-2-[[2-oxo -2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]pentanoyl]amino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-isoindolin-2-yl]-4-oxo-butanoate (0.0438 g, 20.9256 mol, 42.7702% yield) obtained as a white solid. LCMS: (ESI, m/z): [M+2H]⁺=1047.550.

### Step g:

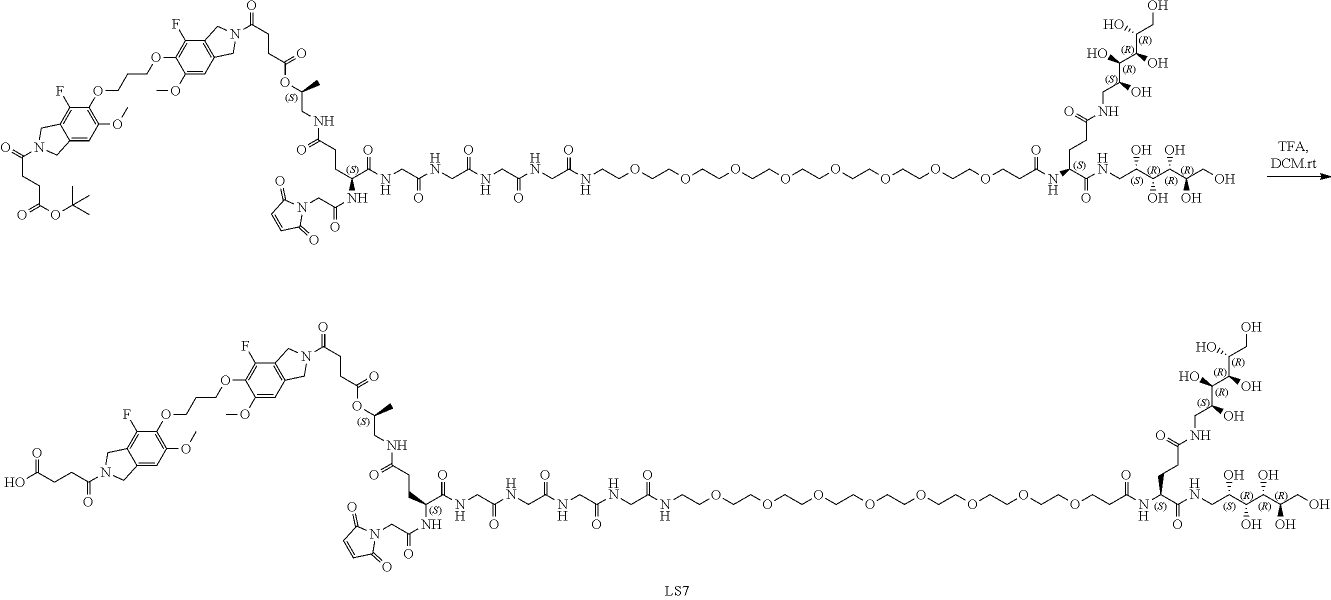

[(1S)-2-[[(4S)-4-[[2-(2,5-dioxopyrrol-1-yl)acetyl]amino]-5-oxo-5- [[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6- pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]pentanoyl]amino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-isoindolin-2-yl]-4-oxo-butanoate (0.039 g, 18.6324 mol) was added to a solution of DCM (4 mL) and TFA (1 mL) at 20° C. under open-air atmosphere. The reaction mixture was stirred for 1 h at 20° C. The reaction mixture was evaporated under reduced pressure. The residue was purified by Prep-HPLC.

The purified fraction was concentrated and dried under vacuo. The residue was purified on C18 column ACN/H₂O (0-50%). The purified fraction was concentrated and dried by lyophilization. There was 4-[5-[3-[2-[4-[(1S)-2-[[(4S)-4-[[2-(2,5-dioxopyrrol-1-yl) acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6- pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]pentanoyl]amino]-1-methyl-ethoxy]-4-oxo-butanoyl]-4-fluoro-6-methoxy-isoindolin-5-yl]oxypropoxy]-4-fluoro-6-methoxy-isoindolin-2-yl]-4-oxo-butanoic acid (0.0148 g, 7.2655 mol, 38.9939% yield ) obtained as a white solid.

LCMS: (ESI, m/z): [M+2H]²⁺=1019.450.

### Example IV-8 Synthesis of Linker-STING Agonist Compound LS8

### Step a:

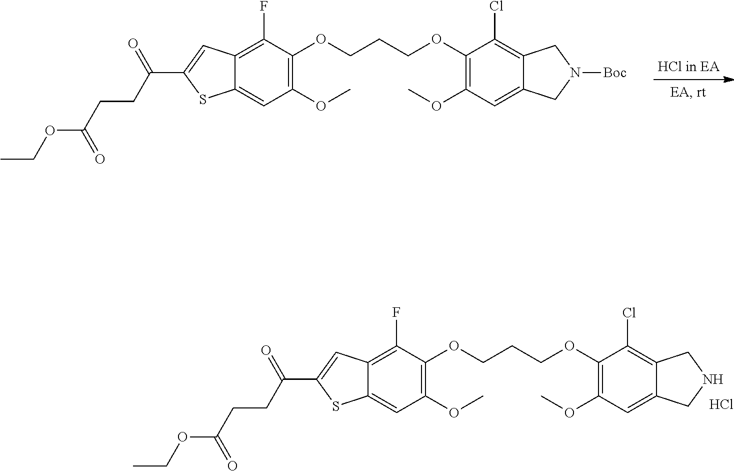 [SMILES: CCOC(=O)CCC(=O)c1cc2c(F)c(OCCCOc3c(OC)cc4c(c3Cl)CN(C(=O)OC(C)(C)C)C4)c(OC)cc2s1 ; CCOC(=O)CCC(=O)c1cc2c(F)c(OCCCOc3c(OC)cc4c(c3Cl)CNC4)c(OC)cc2s1 ; Cl]

HCl in EA (15 mL, 60 mmol) was added to a solution of tert-butyl 4-chloro-5-(3-((2-(4-ethoxy-4-oxobutanoyl)-4-fluoro-6-methoxybenzo[b]thiophen-5-yl)oxy)propoxy)-6-methoxyisoindoline-2-carboxylate (1.53 g, 2.2968 mmol) in Ethyl acetate (10 mL) at 20° C. under open-air atmosphere. The reaction mixture was stirred for 2 h at 20° C. The reaction mixture was evaporated under reduced pressure. There was ethyl 4-[5-[3-(4-chloro-6-methoxy-isoindolin-5-yl)oxypropoxy]-4-fluoro-6-methoxy-benzothiophen-2-yl]-4-oxo-butanoate (1.53 g, 2.7030 mmol, 117.6871% yield) obtained as a brown solid, which was used directly in the next step.

LCMS: (ESI, m/z): [M+H]⁺=565.130. ¹H NMR (400 MHz, DMSO-d₆) δ 8.40 (s, 1H), 7.65 (s, 1H), 7.19 (s, 1H), 4.57 (s, 2H), 4.49 (s, 2H), 4.34 (t, J=6.2 Hz, 2H), 4.24 (t, J=6.1 Hz, 2H), 4.13 (q, J=7.1 Hz, 2H), 3.95 (s, 3H), 3.87 (s, 3H), 3.49-3.41 (m, 2H), 2.73 (t, J=6.3 Hz, 2H), 2.21-2.13 (m, 2H), 1.24 (t, J=7.1 Hz, 3H).

### Step b:

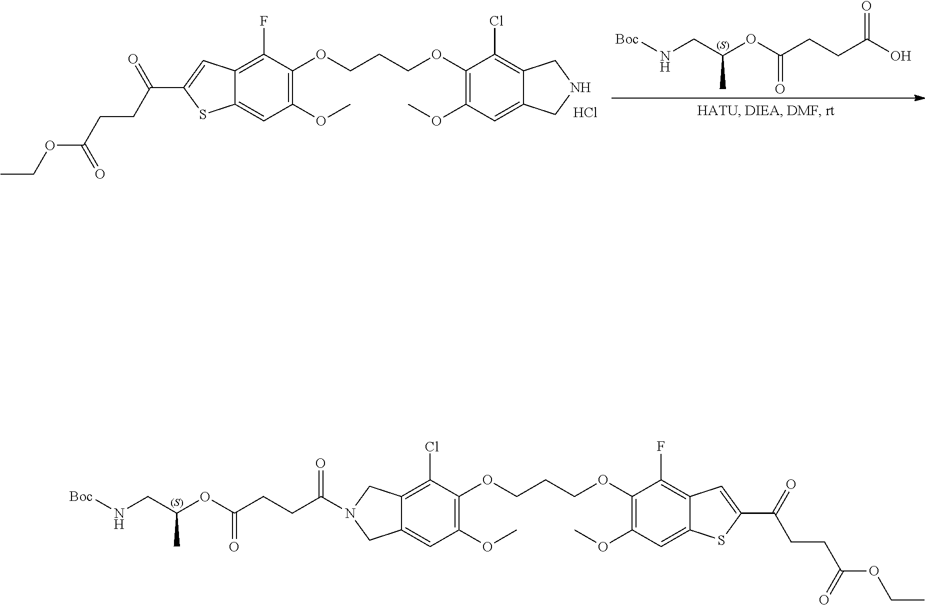

Ethyl-4-[5-[3-(4-chloro-6-methoxy-isoindolin-5-yl)oxypropoxy]-4-fluoro-6-methoxy-benzothiophen-2-yl]-4-oxo-butanoate (0.41 g, 724.3343 mol) and N,N-diisopropylethylamine (0.93 g, 7.1958 mmol) was added to a solution of (S)-4-((1-((tert-butoxycarbonyl)amino)propan-2-yl)oxy)-4-oxobutanoic acid (0.27 g, 980.7590 mol) and HATU (0.39 g, 1.0257 mmol) in DMF (20 mL) at 20° C. under open-air atmosphere. The reaction mixture was stirred for 2 h at 20° C. The reaction mixture was quenched with adding of water (50 mL) at 20° C., extracted with EA (3×50 mL) and brine (50 mL). The organics dried over Na₂SO₄, filtered and evaporated under reduced pressure. The residue was purified on C18 column ACN/H₂O (0.1% FA) (0-80%). The purified fraction was concentrated and dried under vacuo. There was ethyl 4-[5-[3-[2-[4-[(1S)-2- (tert-butoxycarbonylamino)-1-methyl-ethoxy]-4-oxo-butanoyl]-4-chloro-6-methoxy-isoindolin-5-yl]oxypropoxy]-4-fluoro-6-methoxy-benzothiophen-2-yl]-4-oxo-butanoate (0.431 g, 523.4908 mol, 72.2720% yield) obtained as a brown oil. LCMS: (ESI, m/z): [M+H]⁺=823.260.

### Step c:

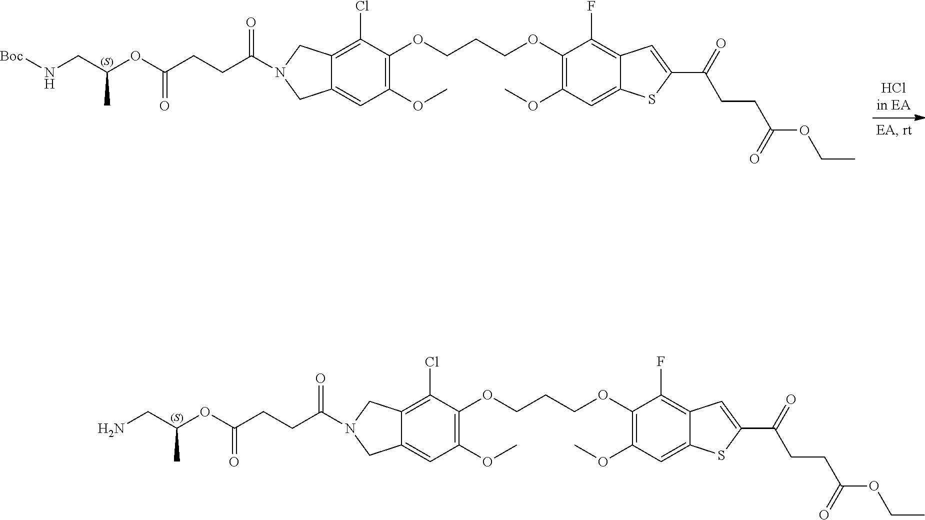

HCl in EA (10 mL, 40 mmol) was added to a solution of ethyl 4-[5-[3-[2-[4-[(1S)-2-(tert-butoxycarbonylamino)-1-methyl-ethoxy]-4-oxo-butanoyl]-4-chloro-6-methoxy-isoindolin-5-yl]oxypropoxy]-4-fluoro-6-methoxy-benzothiophen-2-yl]-4-oxo-butanoate (0.40 g, 485.8384 mol) in EA (5 mL) at 20° C. under open-air atmosphere. The reaction mixture was stirred for 2 h at 20° C. The reaction mixture was evaporated under reduced pressure. The residue was purified on Prep-HPLC. The purified fraction was concentrated and dried under vacuo. The purified fraction was dried by lyophilization. There was ethyl 4-[5-[3-[2-[4-[(1S)-2-amino-1-methyl-ethoxy]-4-oxo-butanoyl]-4-chloro-6-methoxy-isoindolin-5-yl]oxypropoxy]-4-fluoro-6-methoxy-benzothiophen-2-yl]-4-oxo-butanoate (0.096 g, 132.7427 mol, 27.3224% yield) obtained as a white solid. LCMS: (ESI, m/z): [M+H]⁺=723.210.

### Step d:

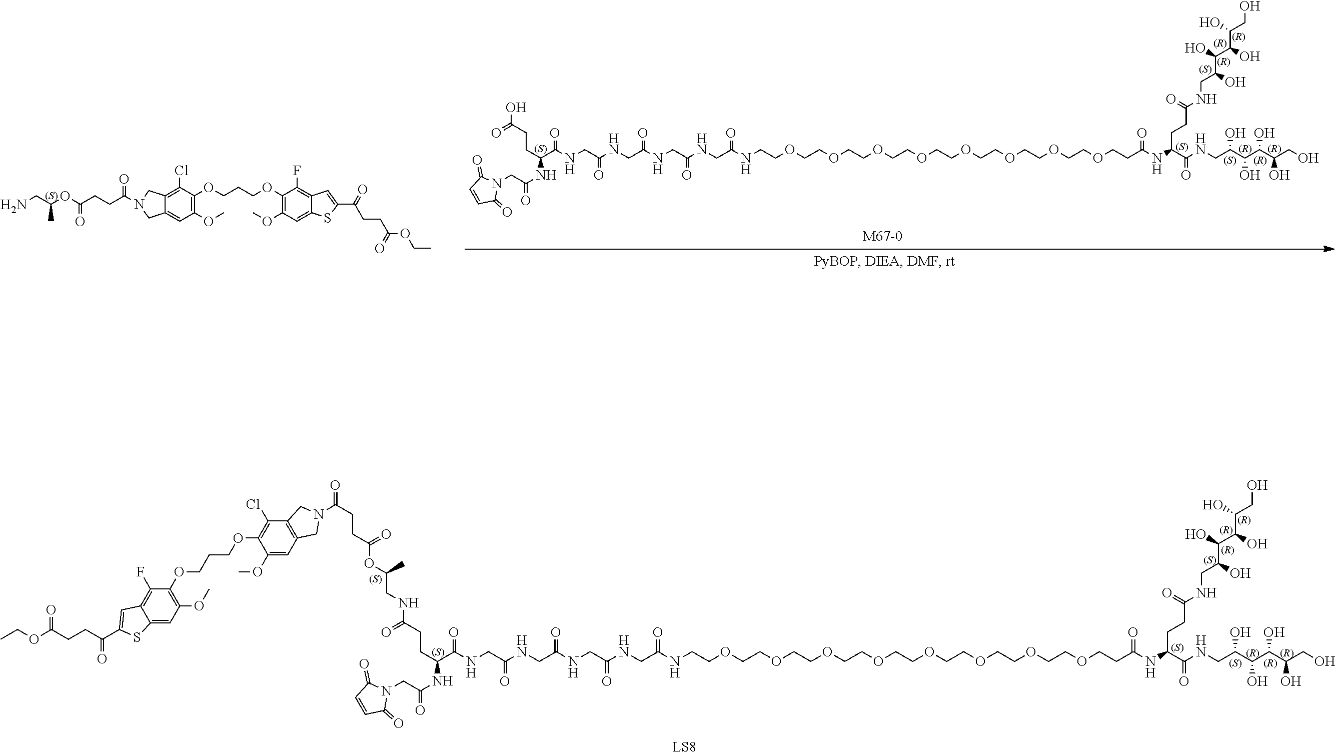

Ethyl-4-[5-[3-[2-[4-[(1S)-2-amino-1-methyl-ethoxy]-4-oxo-butanoyl]-4-chloro-6-methoxy-isoindolin-5-yl]oxypropoxy]-4-fluoro-6-methoxy-benzothiophen-2-yl]-4-oxo-butanoate (0.0292 g, 40.3759 mol) was added to a solution of (4S,47S,53S,54R,55R,56R)-4-(2-(2,5-dioxo-2,5-dihydro-1H-pyrrol-1-yl)acetamido)-53,54,55,56,57-pentahydroxy-5,8,11,14,17,45,50-heptaoxo-47-(((2S,3R,4R,5R)-2,3,4,5,6-pentahydroxyhexyl)carbamoyl)-21,24,27,30,33,36,39,42-octaoxa-6,9,12,15,18,46,51-heptaazaheptapentacontanoic acid (0.0456 g, 32.7732 mol), PyBOP (0.0650 g, 124.9060 mol) and N,N-Diisopropylethylamine (0.0408 g, 315.6859 mol) in DMF (2 mL) at 20° C. under N₂ atmosphere. The reaction mixture was stirred for 2 h at 20° C. The reaction mixture was purified on C18 column ACN/H₂O (0.1% TFA)(0-40%). The purified fraction was dried by lyophilization. The residue was purified on Prep-HPLC. The purified fraction was dried by lyophilization. There was [(1S)-2-[[(4S)-4-[[2-(2,5-dioxopyrrol-1-yl)acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]eth ylamino]ethyl]amino]ethyl]amino]ethyl]amino]ethyl]amino]pentanoyl]amino]-1-methyl-ethyl]-4-[5-[3-[4-chloro-2-(4-ethoxy-4-oxo-butanoyl)-6-methoxy-isoindolin-5-yl]oxypropoxy]-4-fluoro-6-methoxy-benzothiophen-2-yl]-4-oxo-butanoate (0.017 g, 8.1085 mol, 20.0825% yield) obtained as a white solid.

LCMS: (ESI, m/z): [M+2H]²⁺=1049.100.

### Example IV-9 Synthesis of linker-STING Agonist Compound LS9

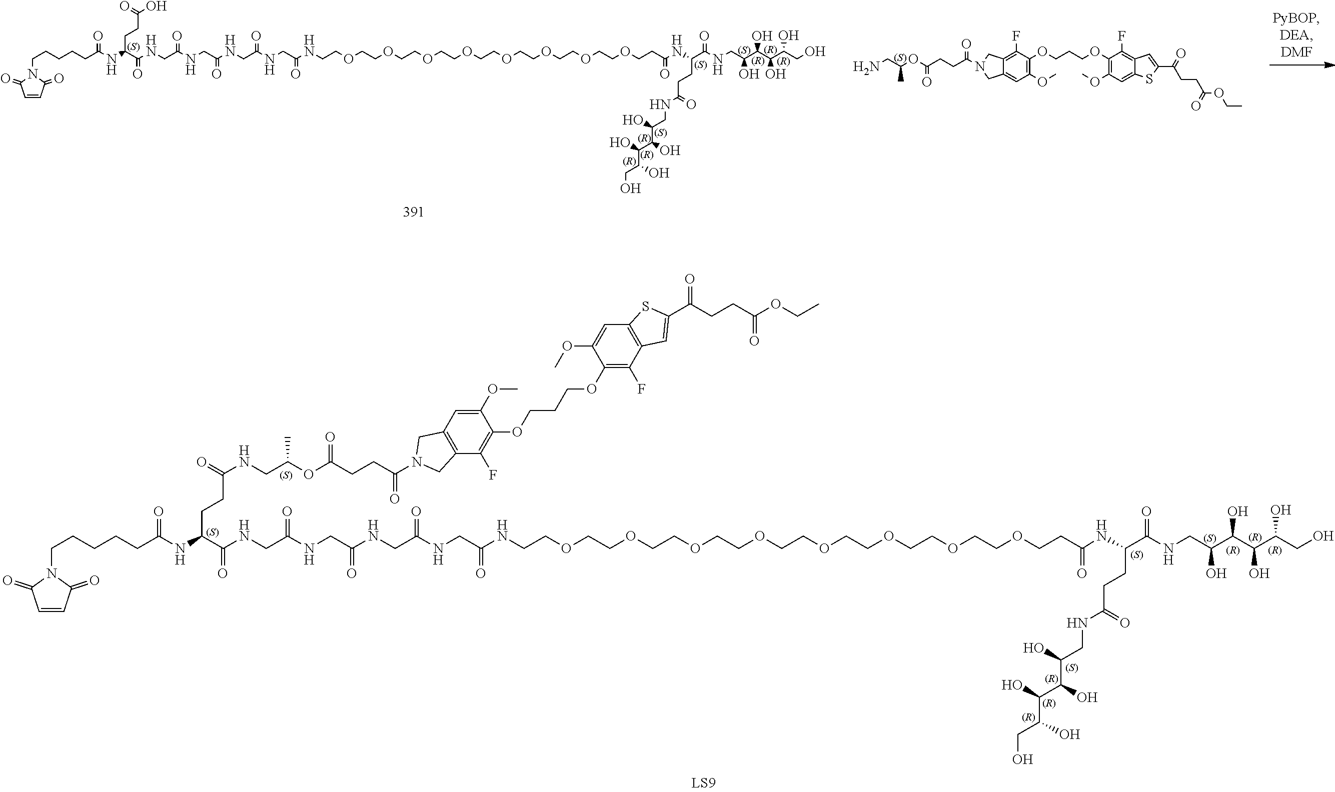

PyBOP (51.0 mg, 98.0032 μmol) was added to a solution of (S)-1-aminopropan-2-yl 4-(5-(3-((2-(4-ethoxy-4-oxobutanoyl)-4-fluoro-6-methoxybenzo[b]thiophen-5-yl)oxy)propoxy)-4-fluoro-6-methoxyisoindolin-2-yl)-4-oxobutanoate,HCl (24.9 mg, 33.5033 gmol), N,N-diisopropylethylamine (30.0 mg, 232.1220 μmol) and (4S,47S,53S,54R,55R,56R)-4-(6-(2,5-dioxo-2,5-dihydro-1H-pyrrol-1-yl)hexanamido)-53,54,55,56,57-pentahydroxy-5,8,11,14,17,45,50-heptaoxo-47-(((2S,3R,4R,5R)-2,3,4,5,6-pentahydroxyhexyl) carbamoyl)-21,24,27,30,33,36,39,42-octaoxa-6,9,12,15,18,46,51-heptaazaheptapentacontanoic acid (43.7 mg, 30.1903 mol) in N,N-dimethylformamide (5 mL) at 25° C. under open-air atmosphere. The reaction mixture was stirred at 25° C. for 1 h. The residue was purified on C-18 column ACN/H₂O(0-100%). The purified fraction was concentrated and dried under vacuo. The residue was purified by HPLC. There was ethyl-4-[5-[3-[2-[4-[(1S)-2-[[(4S)-4-[6-(2,5-dioxopyrrol-1-yl)hexanoylamino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]pentanoyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (33.12 mg, 15.5040 mol, 51.3544% yield) obtained as a white solid.

LCMS: (ESI, m/z): [M+2H]⁺=1068.951.

### Example IV-10 Synthesis of Linker-STING Agonist Compound LS10

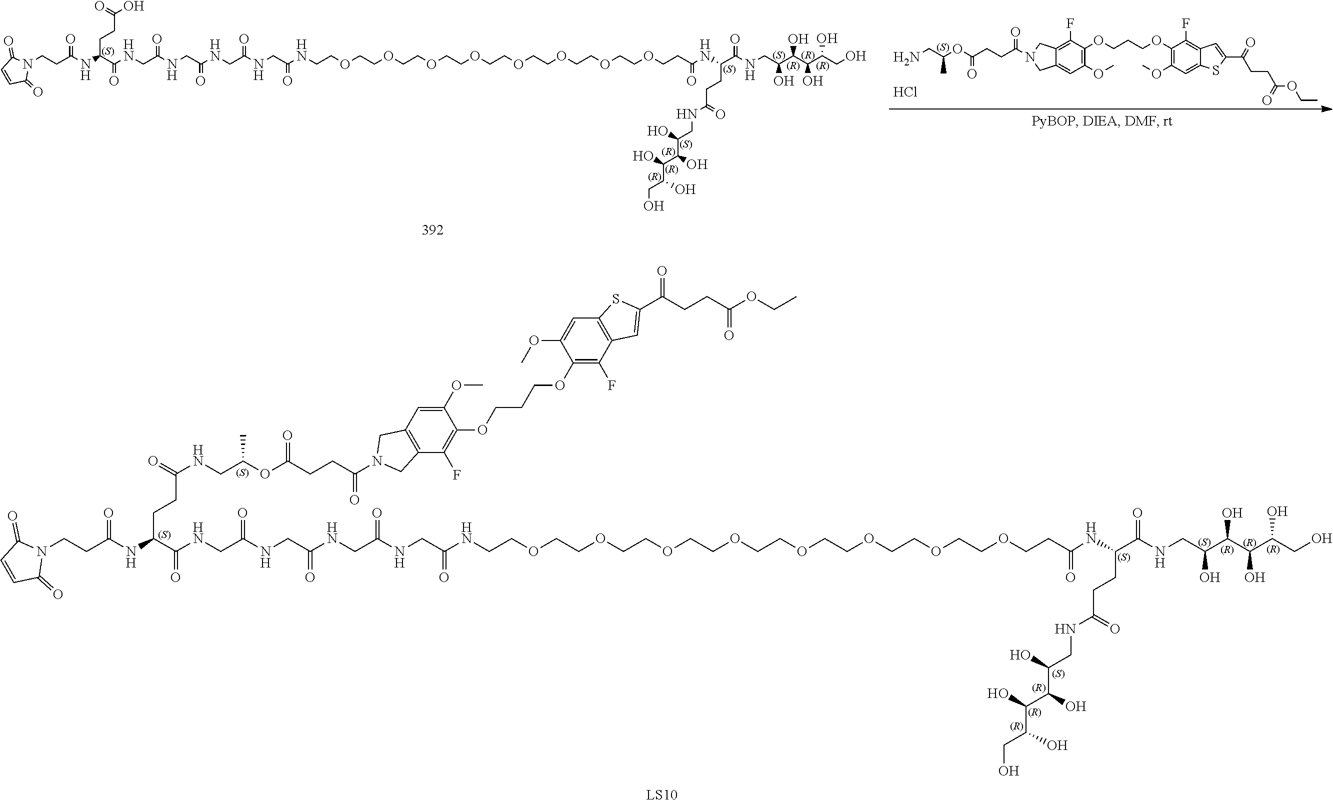

(S)-1-aminopropan-2-yl 4-(5-(3-((2-(4-ethoxy-4-oxobutanoyl)-4-fluoro-6- methoxybenzo[b]thiophen-5-yl)oxy)propoxy)-4-fluoro-6-methoxyisoindolin-2-yl)-4-oxobutanoate hydrochloride (0.0362 g, 48.7076 mol) was added to a solution of (4S,47S,53S,54R,55R,56R)-4-(3-(2,5-dioxo-2,5-dihydro-1H-pyrrol-1-yl)propanamido)-53,54,55,56,57-pentahydroxy-5,8,11,14,17,45,50-heptaoxo-47-(((2S,3R,4R,5R)-2,3,4,5,6-pentahydroxyhexyl)carbamoyl)-21,24,27,30,33,36,39,42-octaoxa-6,9,12,15,18,46,51-heptaazaheptapentacontanoic acid (0.0461 g, 32.8019 mol), PyBOP (0.054 g, 103.7680 mol) and N,N-Diisopropylethylamine (0.0292 g, 225.9320 mol) in DMF (3 mL) at 20° C. under N₂ atmosphere. The reaction mixture was stirred for 1 h at 20° C. The reaction mixture was purified on C18 column ACN/H₂O (0.1% TFA)(0-40%). The purified fraction was dried by lyophilization. The residue was further purified on Prep-HPLC. The purified fraction was dried by lyophilization. There was ethyl 4-[5-[3-[2-[4-[(1S)-2-[[(4S)-4-[3-(2,5-dioxopyrrol-1-yl)propanoylamino]- 5-oxo-5-[[2-oxo-2-[[2-oxo-2-[[2-oxo-2-[[2-oxo-2-[2-[2-[2-[2- [2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2, 3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]pentanoyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.034 g, 16.2358 mol, 49.4965% yield) obtained as a white solid. LCMS: (ESI, m/z): [M+2H]²⁺=1047.850.

### Example IV-11 Synthesis of Linker-STING Agonist Compound LS11

### Step a:

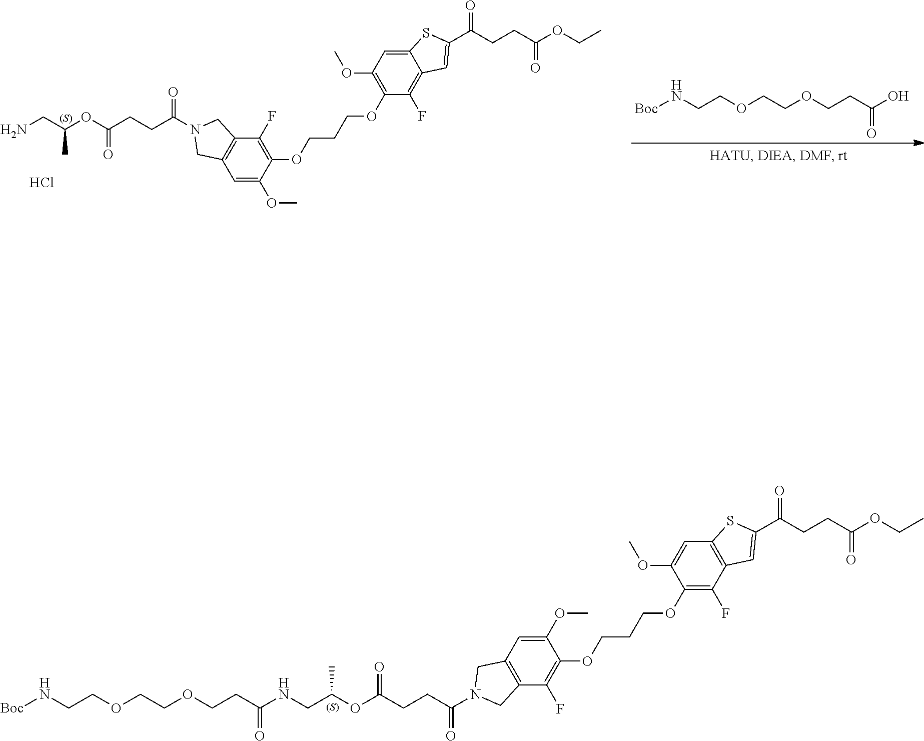

(S)-1-aminopropan-2-yl 4-(5-(3-((2-(4-ethoxy-4-oxobutanoyl)-4-fluoro-6- methoxybenzo[b]thiophen-5-yl)oxy)propoxy)-4-fluoro-6-methoxyisoindolin-2-yl)-4-oxobutanoate hydrochloride (0.048 g, 65.6611 mol) and N,N-diisopropylethylamine (0.0452 g, 349.7304 mol) were added to a solution of 2,2-dimethyl-4-oxo-3,8,11-trioxa-5-azatetradecan-14-oic acid (0.0363 g, 130.8989 mol) and HATU (0.0548 g, 144.1236 mol) in DMF (2 mL) at 20° C. under N₂ atmosphere. The reaction mixture was stirred for 2 h at 20° C. The reaction mixture was purified on C18 column ACN/H₂O (0.1% TFA)(0-65%). The purified fraction was concentrated and dried under vacuo. There was ethyl 4-[5-[3-[2-[4-[(1S)-2-[3-[2-[2-(tert-butoxycarbonylamino)ethoxy]ethoxy]propanoylamino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.049 g, 50.7222 mol, 77.2484% yield) obtained as a white solid. LCMS: (ESI, m/z): [M+H]⁺=966.380.

### Step b:

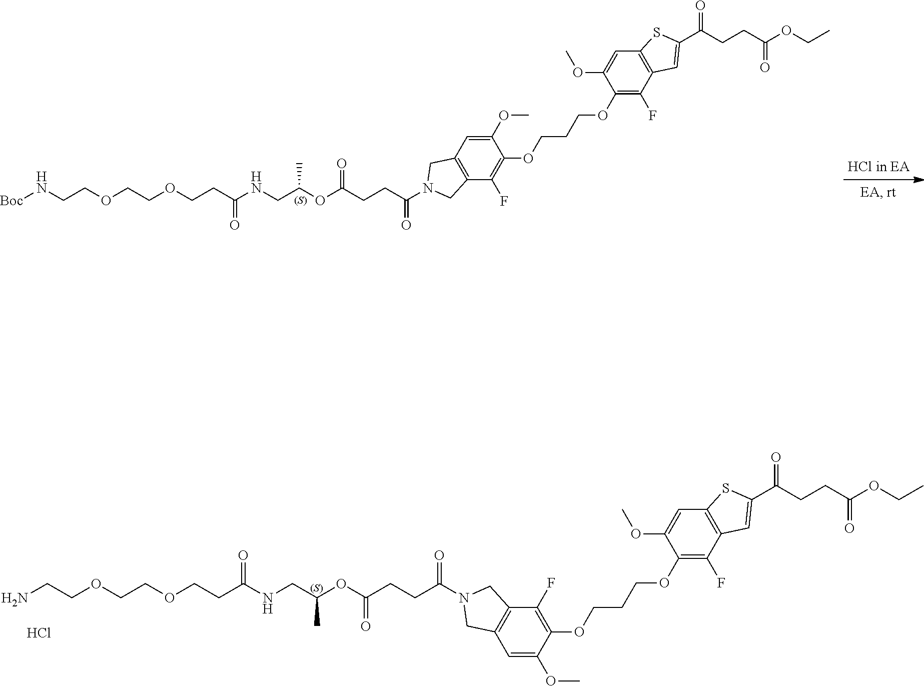

HCl in EA(5 mL, 20 mmol) was added to a solution of ethyl 4-[5-[3-[2-[4-[(1S)-2-[3-[2-[2-(tert-butoxycarbonylamino)ethoxy]ethoxy]propanoylamino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.049 g, 50.7222 mol) in EA (2 mL) at 20° C. under open-air atmosphere. The reaction mixture was stirred for 1 h at 20° C. The reaction mixture was evaporated under reduced pressure. The residue was dried by lyophilization. There was ethyl 4-[5-[3-[2-[4-[(1S)-2-[3-[2-(2-aminoethoxy)ethoxy]propanoylamino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.0433 g, 47.9836 mol, 94.6008% yield) obtained as a white solid. LCMS: (ESI, m/z): [M+H]⁺=866.330.

### Step c:

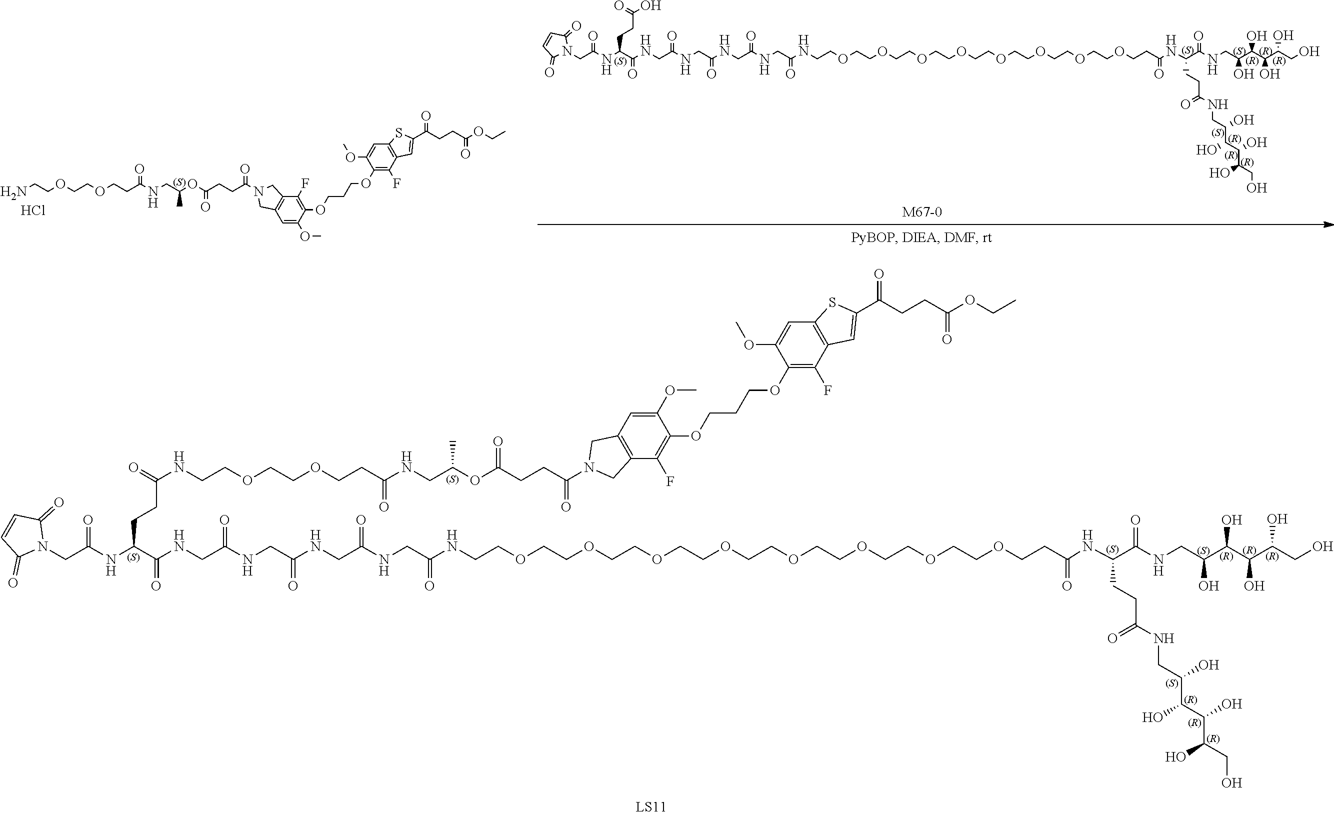

(S)-1-(3-(2-(2-aminoethoxy)ethoxy)propanamido)propan-2-yl 4-(5-(3-((2-(4-ethoxy-4-oxobutanoyl)-4-fluoro-6-methoxybenzo[b]thiophen-5-yl)oxy)propoxy)-4-fluoro-6-methoxyisoindolin-2-yl)-4-oxobutanoate hydrochloride (0.0290 g, 33.4899 mol) was added to a solution of (4S,47S,53S,54R,55R,56R)-4-(2-(2,5-dioxo-2,5-dihydro-1H-pyrrol-1-yl)acetamido)-53,54,55,56,57-pentahydroxy-5,8,11,14,17,45,50-heptaoxo-47-(((2S,3R,4R,5R)-2,3,4,5,6-pentahydroxyhexyl)carbamoyl)-21,24,27,30,33,36,39,42-octaoxa-6,9,12,15,18,46,51-heptaazaheptapentacontanoic acid (0.0373 g, 26.8079 mol), PyBOP (0.0407 g, 78.2104 μmol) and N,N-Diisopropylethylamine (0.0302 g, 233.6694 mol) in DMF (2 mL) at 20° C. under N₂ atmosphere. The reaction mixture was stirred for 2 h at 20° C. The reaction mixture was purified on C18 column ACN/H₂O (0.1% TFA)(0-35%). The purified fraction was dried by lyophilization. The residue was purified on Prep-HPLC. The purified fraction was dried by lyophilization. There was ethyl 4-[5-[3-[2-[4-[(1S)-2-[3-[2-[2-[[(4S)-4-[[2-(2,5-dioxopyrrol-1-yl)acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]pentanoyl]amino]ethoxy]ethoxy]propanoylamino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.0166 g, 7.4130 mol, 27.6524% yield) obtained as a white solid. LCMS: (ESI, m/z): [M+2H]²⁺=1120.600.

### Example IV-12 Synthesis of Linker-STING Agonist Compound LS12

### Step a:

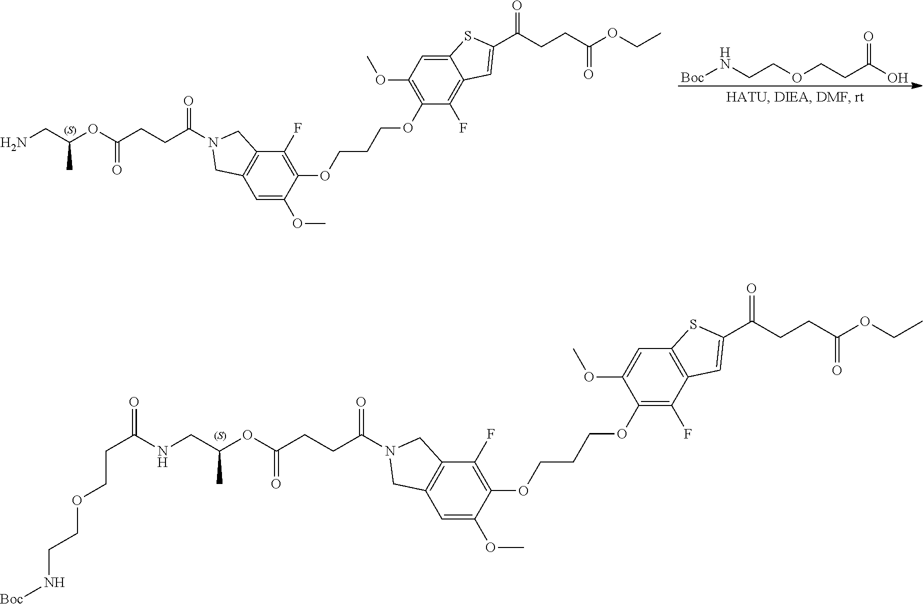

(S)-1-aminopropan-2-yl 4-(5-(3-((2-(4-ethoxy-4-oxobutanoyl)-4-fluoro-6- methoxybenzo[b]thiophen-5-yl)oxy)propoxy)-4-fluoro-6-methoxyisoindolin-2-yl)-4-oxobutanoate hydrochloride (0.053 g, 71.3123 mol) and N,N-diisopropylethylamine (0.054 g, 417.8195 mol) were added to a solution of 3-(2-((tert-butoxycarbonyl)amino)ethoxy)propanoic acid (0.039 g, 167.1947 mol) and HATU (0.050 g, 131.4996 mol) in DMF (2 mL) at 20° C. under open-air atmosphere. The reaction mixture was stirred for 2 h at 20° C. The reaction mixture was purified on C18 column ACN/H₂O (0.1% TFA)(0-65%). The purified fraction was concentrated and dried under vacuo. There was ethyl 4-[5-[3-[2-[4-[(1S)-2-[3-[2-(tert-butoxycarbonylamino)ethoxy]propanoylamino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.050 g, 54.2303 μmol, 76.0462% yield) obtained as a white solid. LCMS: (ESI, m/z): [M+H]⁺=922.350.

### Step b:

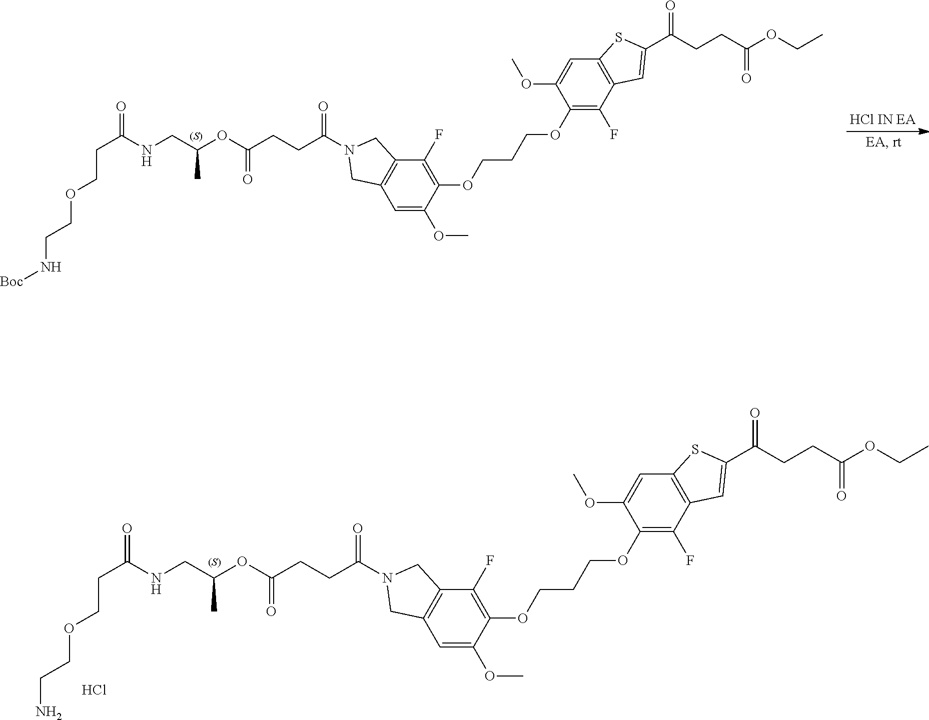

HCl in EA (2 mL, 8 mmol) was added to a solution of ethyl 4-[5-[3-[2-[4-[(1S)-2-[3-[2-(tert-butoxycarbonylamino)ethoxy]propanoylamino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.049 g, 53.1456 μmol) in EA (1 mL) at 20° C. under open-air atmosphere. The reaction mixture was stirred for 1 h at 20° C. The reaction mixture was evaporated under reduced pressure. The residue was purified on C18 column ACN/H₂O (0.1% TFA)(0-50%). The purified fraction was concentrated and dried under vacuo. The purified fraction was dried by lyophilization. There was (S)-1-(3-(2-aminoethoxy)propanamido)propan-2-yl-4-(5-(3-((2-(4-ethoxy-4-oxobutanoyl)-4-fluoro-6-methoxybenzo [b]thiophen-5-yl)oxy)propoxy)-4-fluoro-6-methoxyisoindolin-2-yl)-4-oxobutanoate hydrochloride (0.040 g, 48.6690 mol, 91.5766% yield) obtained as a white solid. LCMS: (ESI, m/z): [M+H]⁺=822.300.

### Step c:

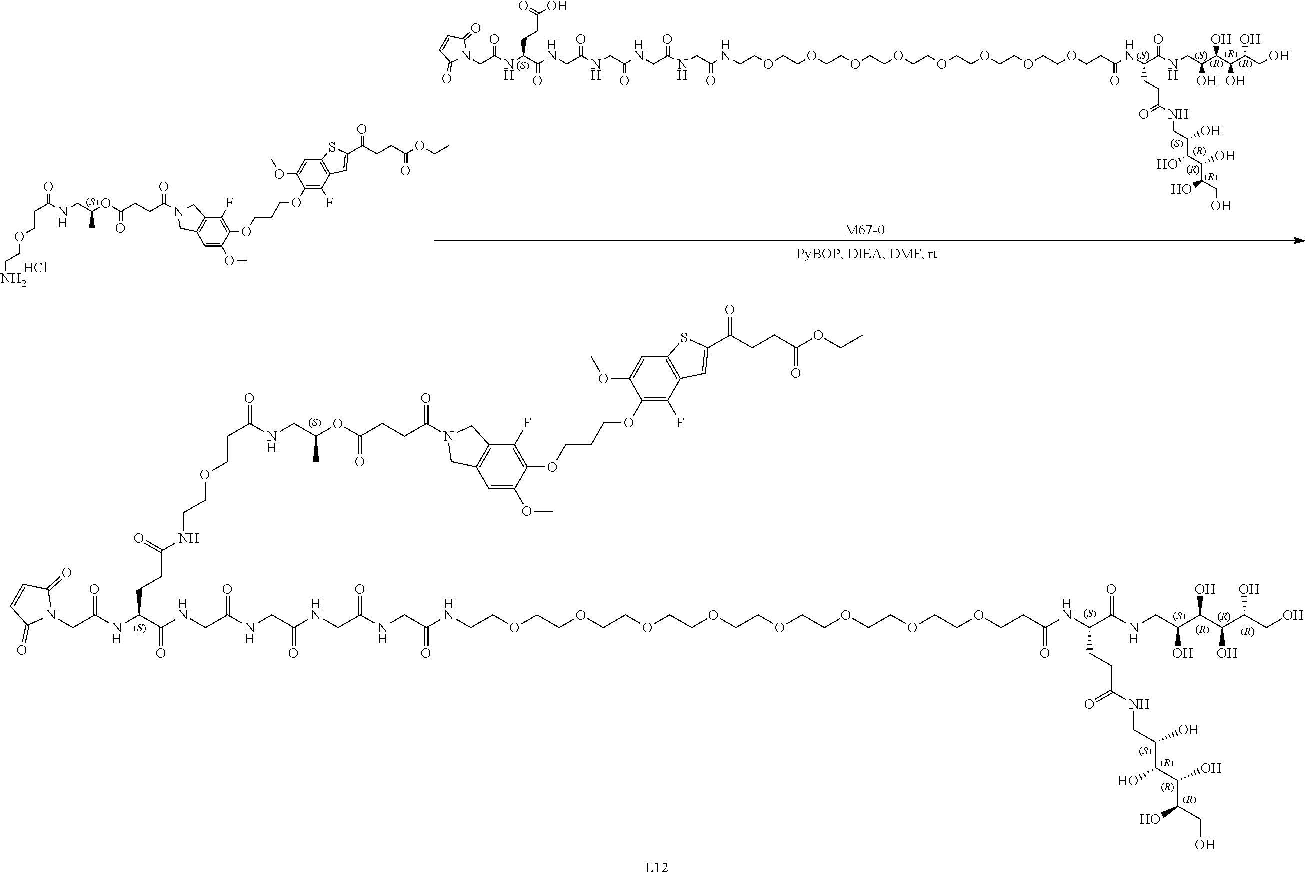

(S)-1-(3-(2-aminoethoxy)propanamido)propan-2-yl-4-(5-(3-((2-(4-ethoxy-4-oxobutanoyl)-4-fluoro-6-methoxybenzo[b]thiophen-5-yl)oxy)propoxy)-4-fluoro-6-methoxyisoindolin-2-yl)-4-oxobutanoate hydrochloride (0.0464 g, 0.05646 mmol) was added to a solution of (4S,47S,53S,54R,55R,56R)-4-(2-(2,5-dioxo-2,5-dihydro-1H-pyrrol-1-yl)acetamido)-53,54,55,56,57-pentahydroxy-5,8,11,14,17,45,50-heptaoxo-47-(((2S,3R,4R,5R)-2,3,4,5,6-pentahydroxyhexyl)carbamoyl)-21,24,27,30,33,36,39,42-octaoxa-6,9,12,15,18,46,51-heptaazaheptapentacontanoic acid (0.0647 g, 0.0465 mmol), PyBOP (0.0630 g, 0.1211 mmol) and N,N-diisopropylethylamine (0.0647 g, 0.5006 mmol) in DMF (2 mL) at 20° C. under N₂ atmosphere. The reaction mixture was stirred for 2 h at 20° C. The reaction mixture was purified on C18 column ACN/H₂O (0.1% TFA)(0-40%). The purified fraction was dried by lyophilization. The residue was purified on Prep-HPLC. The purified fraction was dried by lyophilization. There was ethyl 4-[5-[3-[2-[4-[(1S)-2-[3-[2-[[(4S)-4-[[2-(2,5-dioxopyrrol-1-yl)acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]pentanoyl]amino]ethoxy]propanoylamino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.0342 g, 15.5791 μmol, 27.60% yield) obtained as a white solid. LCMS: (ESI, m/z): [M+2H]²⁺=1098.600.

### Example IV-13 Synthesis of Linker-STING Agonist Compound LS13

### Step a:

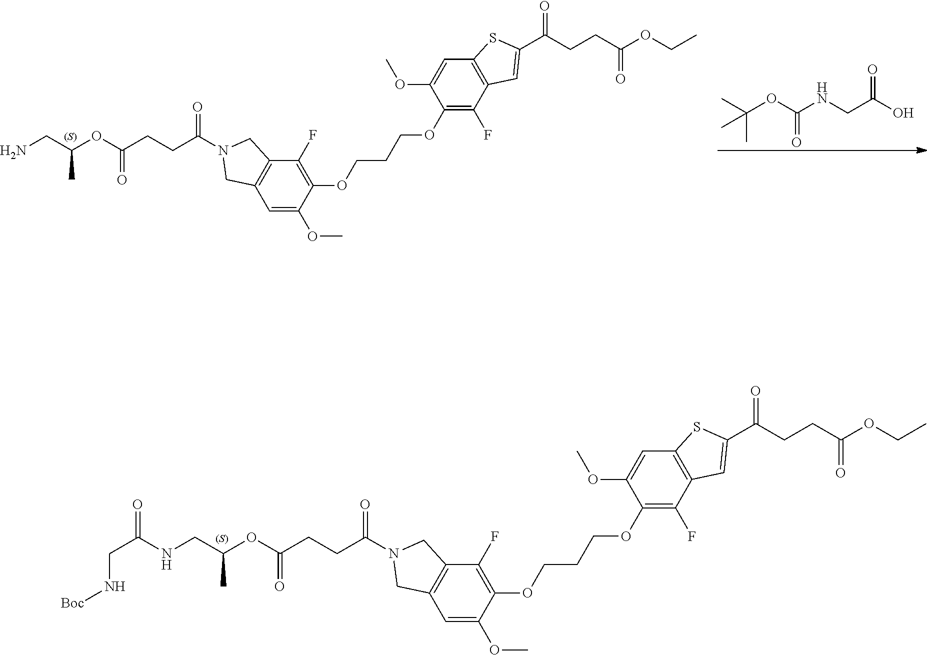

HATU (0.047 g, 123.6096 mol) was added to a solution of (S)-1-aminopropan-2-yl 4-(5-(3-((2-(4-ethoxy-4-oxobutanoyl)-4-fluoro-6-methoxybenzo[b]thiophen-5-yl)oxy)propoxy)-4-fluoro-6-methoxyisoindolin-2-yl)-4-oxobutanoate, HCl (0.058 g, 78.0399 gmol), N,N-diisopropylethylamine (0.030 g, 232.1220 mol) and BOC-Glycine (0.019 g, 108.4587 mol) in N,N-Dimethylformamide (2 mL) at 25° C. under open-air atmosphere. The reaction mixture was stirred at 25° C. for 1h. The residue was purified on C-18 column ACN/H₂O(0-100%). There was ethyl 4-[5-[3-[2-[4-[(1S)-2-[[2-(tert-butoxycarbonylamino) acetyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.07 g, 81.0264 μmol, 103.8270% yield) obtained as a white solid. LCMS: (ESI, m/z): [M+H]⁺=864.311.

### Step b:

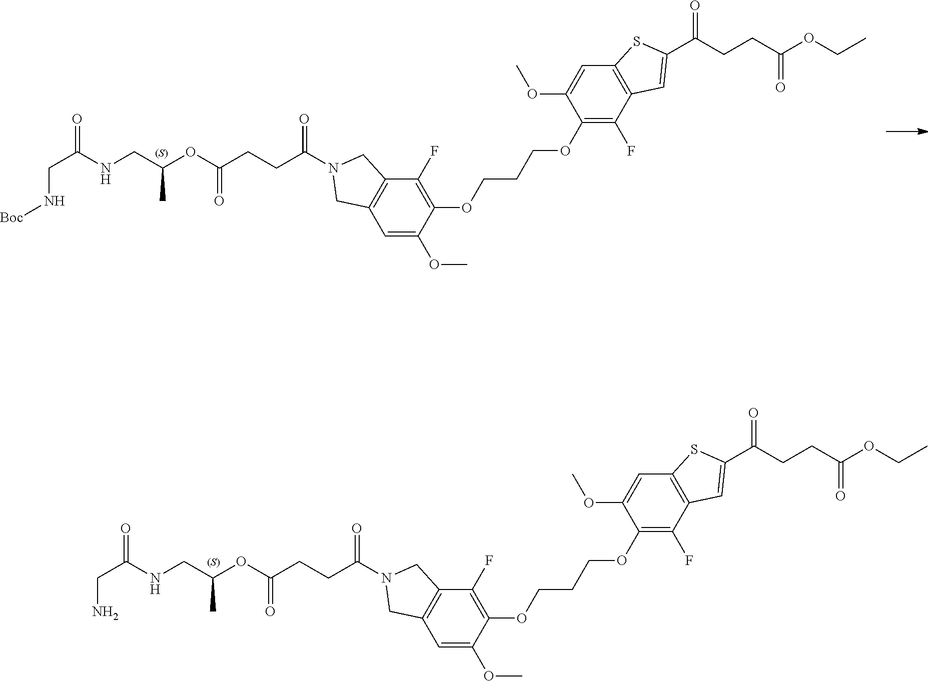

HCl (2 mL, 4M in EA) was added to a solution of ethyl 4-[5-[3-[2-[4-[(1S)-2-[[2-(tert-butoxycarbonylamino)acetyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.07 g, 81.0264 mol) in EA (5 mL) at 25° C. under open-air atmosphere. The reaction mixture was stirred for lh. The reaction mixture was evaporated under reduced pressure. The residue was purified on C-18 column ACN/H₂O(0-100%). The purified fraction was concentrated and dried under vacuo. There was ethyl 4-[5-[3-[2-[4-[(1S)-2-[(2-aminoacetyl)amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (52 mg, 68.0806 mol, 84.0228% yield) obtained as a white solid. LCMS: (ESI, m/z): [M+H]⁺=764.259.

### Step c:

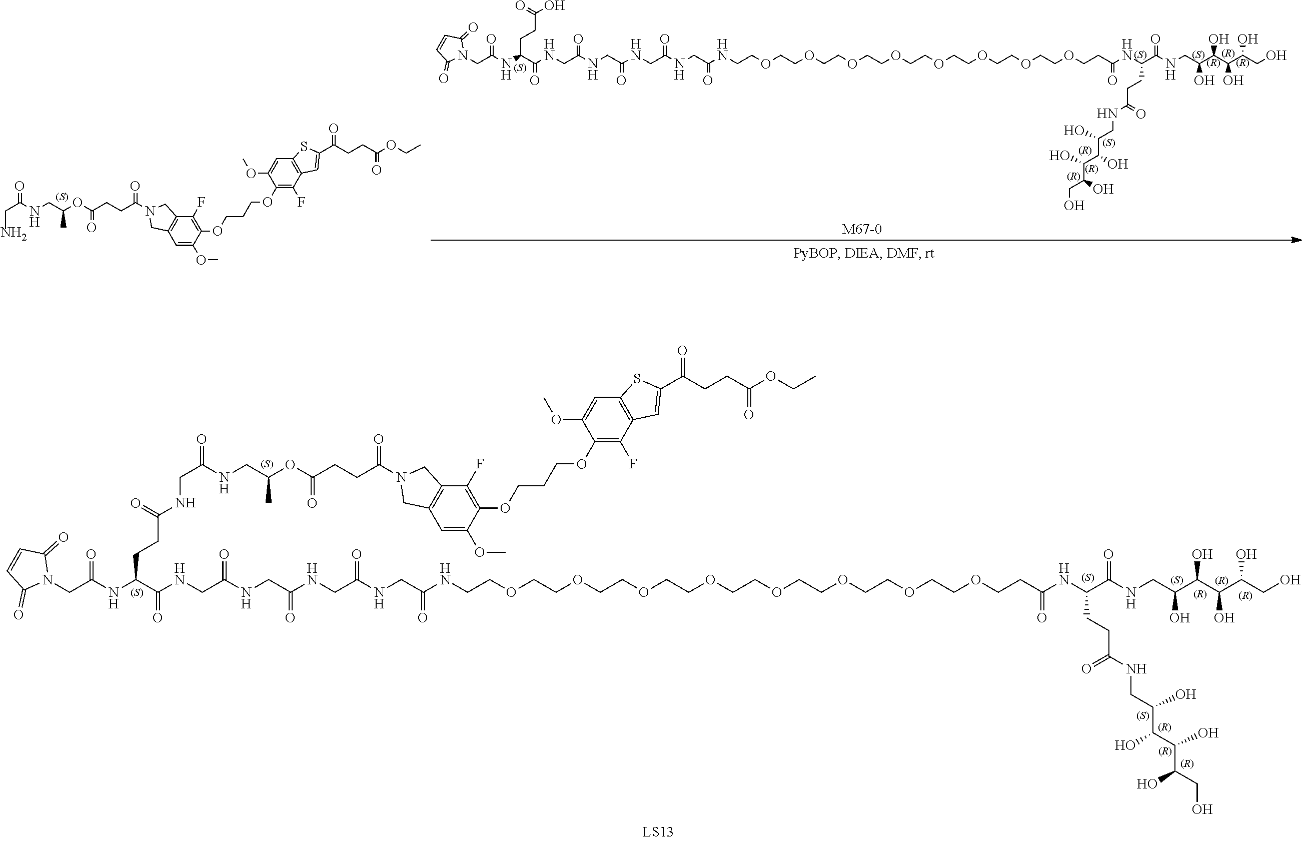

PyBOP (38.7000 mg, 74.3671 μmol) was added to a solution of ethyl 4-[5-[3-[2-[4-[(1S)-2-[(2-aminoacetyl)amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.0297 g, 38.8845 mol) (4S,47S,53S,54R,55R,56R)-4-(2-(2,5-dioxo-2,5-dihydro-1H-pyrrol-1-yl)acetamido)-53,54,55,56,57-pentahydroxy-5,8,11,14,17,45,50-heptaoxo-47-(((2S,3R,4R,5R)-2,3,4,5,6-pentahydroxyhexyl)carbamoyl)-21,24,27,30,33,36,39,42-octaoxa-6,9,12,15,18,46,51-heptaazaheptapentacontanoic acid (0.0497 g, 35.7199 mol) and N,N-diisopropylethylamine (0.0250 g, 193.4350 mol) in N,N-dimethylformamide (5 mL) at 25° C. under open-air atmosphere. The reaction mixture was stirred at 25° C. for lh. The residue was purified on C-18 column ACN/H20(0-100%). The residue was purified by HPLC. There was ethyl 4-[5-[3-[2-[4-[(1S)-2-[[2-[[(4S)-4-[[2-(2,5-dioxopyrrol-1-yl)acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6- pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]eth ylamino]ethyl]amino]ethyl]amino]ethyl]amino]ethyl]amino]pentanoyl]amino]acetyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.0213 g, 9.9665 mol, 25.6310% yield) obtained as a white solid.

LCMS: (ESI, m/z): [M+2H]²⁺=1068.929

### Example IV-14 Synthesis of Linker-STING Agonist Compound LS14

### Step a:

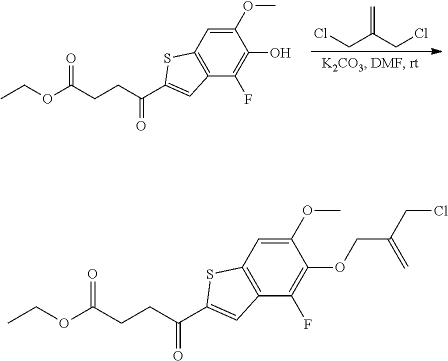

K₂CO₃ (0.274 g, 1.9826 mmol) was added to a solution of ethyl 4-(4-fluoro-5-hydroxy-6-methoxybenzo[b]thiophen-2-yl)-4-oxobutanoate (0.216 g, 661.8876 mol) and 3-Chloro-2-(chloromethyl)prop-1-ene (324.0000 mg, 2.5921 mmol) in N,N-dimethylformamide (5 mL) at 25° C. The reaction mixture was stirred for 5 h at 25° C. The resulting reaction mixture was diluted with H₂O(20 mL), and then extracted with EA (200 mL). The organic layer was separated and washed with brine (20×2 mL). The organic layer was collected and dried over Na₂SO₄, filtered and evaporated under reduced pressure. The residue was purified by silica gel column chromatography, eluting with ethyl acetate/n-hexane (0-50%). The purified fraction was concentrated and dried under reduced pressure. There was ethyl 4-[5-[2-(chloromethyl)allyloxy]-4-fluoro-6-methoxy-benzothiophen-2-yl]-4-oxo-butanoate (0.247 g, 595.3605 mol, 89.9489% yield) obtained as a white solid. LCMS: (ESI, m/z): [M+H]⁺=414.872.

### Step b:

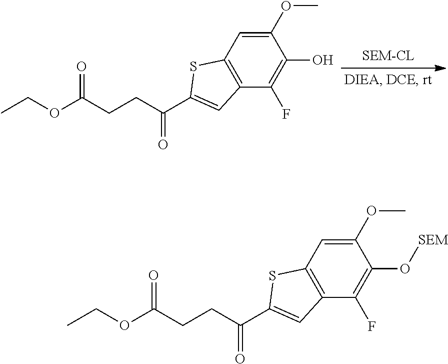

(2-(Chloromethoxy)ethyl)trimethylsilane (635.8500 mg, 3.8139 mmol) was added to a solution of ethyl 4-(4-fluoro-5-hydroxy-6-methoxybenzo[b]thiophen-2-yl)-4-oxobutanoate (0.413 g, 1.2656 mmol), N,N-diisopropylethylamine (656.6700 mg, 5.0809 mmol) in ethylene chloride (6 mL) at 25° C. The reaction mixture was stirred for 2 h at 25° C. The reaction mixture was concentrated and diluted with water (30 mL), washed with DCM (2×30 mL). The organics were dried over Na₂SO₄, filtered and evaporated under reduced pressure. There was ethyl 4-[4-fluoro-6-methoxy-5-(2-trimethylsilylethoxymethoxy)benzothiophen-2-yl]-4-oxo-butanoate (0.54 g, 1.1827 mmol, 93.4498% yield, Lot:20220310) obtained as a yellow solid. LCMS: (ESI, m/z): [M+H]⁺=546.600.

### Step c:

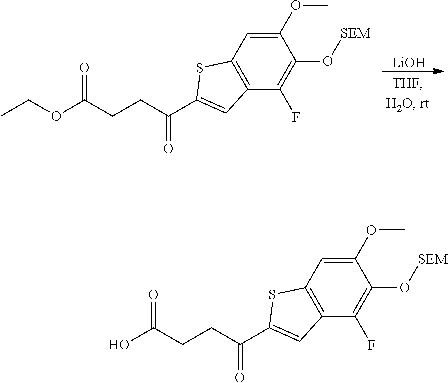 [SMILES: CCOC(=O)CCC(=O)c1cc2c(F)c(OCOCC[Si](C)(C)C)c(OC)cc2s1 ; "LiOH" ; COc1cc2sc(C(=O)CCC(=O)O)cc2c(F)c1OCOCC[Si](C)(C)C]

Ethyl-4-(4-fluoro-6-methoxy-5-((2-(trimethylsilyl)ethoxy)methoxy)benzo[b]thiophen-2-yl)-4-oxobutanoate (0.53 g, 1.1608 mmol) was added to a solution of LiOH (0.14 g, 5.8459 mmol), tetrahydrofura (10 mL) and water (8 mL) under stirring at 25° C. The reaction mixture was stirred for 2 h at 25° C. pH was adjusted to 5 with HCl (1 M). The reaction mixture was concentrated and diluted with EA (15 mL), washed with water (2×15 mL) and brine (15 mL). The organics were dried over Na₂SO₄, filtered and evaporated under reduced pressure. The residue was purified on C18 column MeCN/Water (0-100%). The purified fraction was concentrated and dried under vacuo. There was 4-[4-fluoro-6-methoxy-5-(2-trimethylsilylethoxymethoxy)benzothiophen-2-yl]-4-oxo-butanoic acid (0.276 g, 644.0381 mol, 55.4844% yield) obtained as a yellow solid. LCMS: (ESI, m/z): [M+H]⁺=428.546.

### Step d:

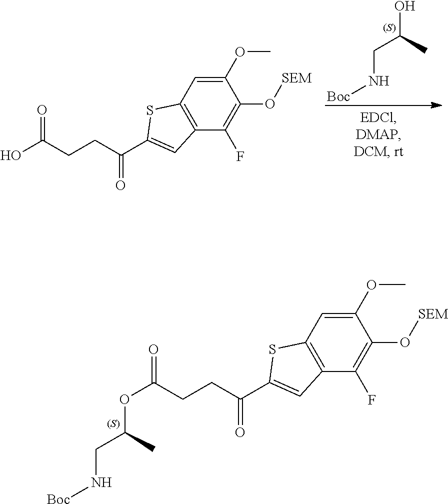

N-(4-pyridyl)dimethylamine (0.026 g, 212.8234 mol) was added to a solution of 4-(4-fluoro-6-methoxy-5-((2-(trimethylsilyl)ethoxy)methoxy)benzo[b]thiophen-2-yl)-4-oxobutanoic acid (0.257 g, 599.7021 mol), N-(4-pyridyl)dimethylamine (0.026 g, 212.8234 mol), EDCI (0.388 g, 2.0240 mmol) and tert-butyl N-[(2S)-2-hydroxypropyl]carbamate (0.470 g, 2.6823 mmol) in dichloromethane (5 mL) at 25° C. The reaction mixture was stirred for 3 h at 25° C. The reaction mixture was evaporated under reduced pressure. The residue was purified on C18 column MeCN/Water (0-75%). The purified fraction was concentrated and dried under vacuo. There was [(1S)-2-(tert-butoxycarbonylamino)-1-methyl-ethyl]4-[4-fluoro-6-methoxy-5-(2-trimethylsilylethoxymethoxy)benzothiophen-2-yl]-4-oxo-butanoate (0.063 g, 107.5534 mol, 17.9345% yield) obtained as a brown solid. LCMS: (ESI, m/z): [M+H]⁺=585.759.

### Step e:

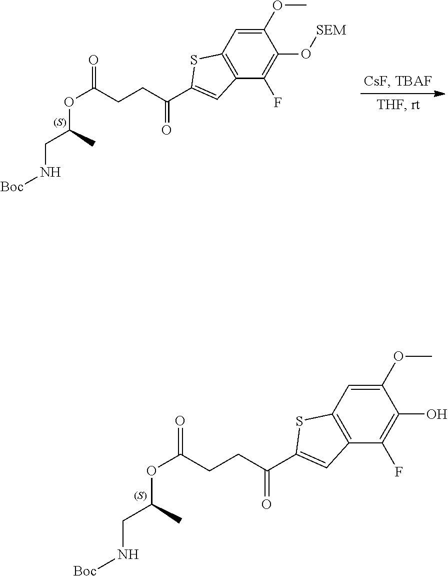

TBAF (9.5300 mg, 36.4491 mol) was added to a solution of (S)-1-((tert-butoxycarbonyl)amino)propan-2-yl-4-(4-fluoro-6-methoxy-5-((2-(trimethylsilyl)ethoxy)methoxy)benzo[b]thiophen-2-yl)-4-oxobutanoate (0.210 g, 358.5112 mol) and CsF (0.0241 g, 158.6535 mol) in tetrahydrofura (8 mL) at 25° C. The reaction mixture was stirred at 13° C. The reaction mixture was evaporated under reduced pressure. The residue was purified on silica gel column MeCN/Water (0-50%). The purified fraction was concentrated and dried under vacuo. There was [(1S)-2-(tert-butoxycarbonylamino)-1-methyl-ethyl]-4-(4-fluoro-5-hydroxy-6-methoxy-benzothiophen-2-yl)-4-oxo-butanoate (0.077 g, 169.0465 mol, 47.1524% yield) obtained as a yellow solid.

LCMS: (ESI, m/z): [M+H]⁺=455.497.

### Step f:

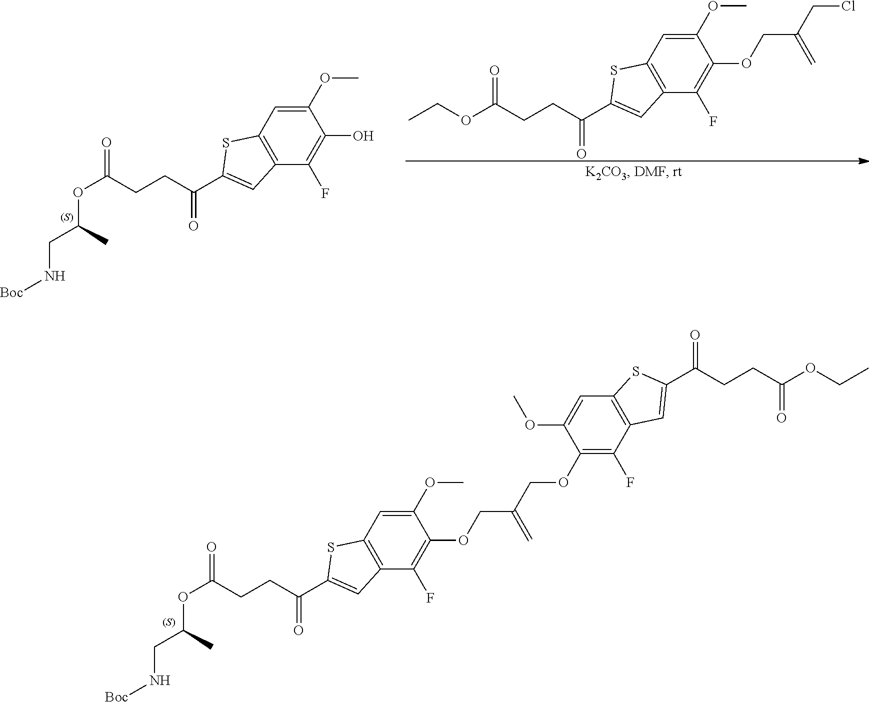

K₂CO₃ (0.063 g, 455.8429 mol) was added to a solution of (S)-1-((tert-butoxycarbonyl)amino)propan-2-yl-4-(4-fluoro-5-hydroxy-6-methoxybenzo[b]thiophen-2-yl)-4- oxobutanoate (0.065 g, 142.7016 mol), ethyl-4-(5-((2-(chloromethyl)allyl)oxy)-4-fluoro-6- methoxybenzo[b]thiophen-2-yl)-4-oxobutanoate (0.085 g, 204.8811 mol) in N,N-Dimethylformamide (4 mL) at 25° C. The reaction mixture was stirred at 25° C. The residue was purified on C18 column MeCN/Water (0-75%). The purified fraction was concentrated and dried under vacuo. There was ethyl 4-[5-[2-[[2-[4-[(1S)-2-(tert-butoxycarbonylamino)-1-methyl-ethoxy]-4-oxo-butanoyl]-4-fluoro-6-methoxy-benzothiophen-5-yl]oxymethyl]allyloxy]-4-fluoro-6-methoxy-benzothiophen-2-yl]-4-oxo-butanoate (0.054 g, 64.7552 mol, 45.3781% yield) obtained as a yellow solid. LCMS: (ESI, m/z): [M+H]⁺=833.912.

### Step g:

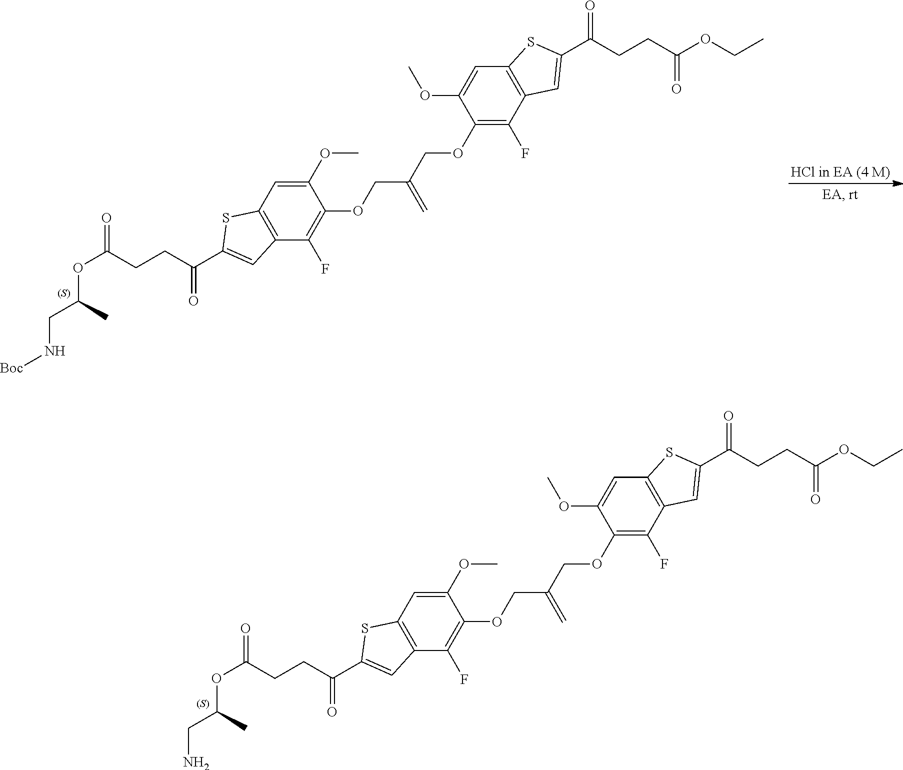

HCl (4 M in EA) (10 ml) was added to a solution of (S)-1-((tert-butoxycarbonyl)amino)propan-2-yl-4-(5-((2-(((2-(4-ethoxy-4-oxobutanoyl)-4-fluoro-6- methoxybenzo[b]thiophen-5-yl)oxy)methyl)allyl)oxy)-4-fluoro-6-methoxybenzo[b]thiophen-2-yl)-4-oxobutanoate (0.050 g, 59.9585 mol) in EA (5 mL) at 25° C. The reaction mixture was stirred for 16 h at 25° C. The reaction mixture was evaporated under reduced/vacuo pressure. There was ethyl 4-[5-[2-[[2-[4-[(1S)-2-amino-1-methyl-ethoxy]-4-oxo-butanoyl]-4-fluoro-6-methoxy-benzothiophen-5-yl]oxymethyl]allyloxy]-4-fluoro-6-methoxy-benzothiophen-2-yl]-4-oxo-butanoate (0.038 g, 51.7856 mol, 86.3691% yield) obtained as a off-white solid. LCMS: (ESI, m/z): [M+H]⁺=733.795.

### Step h:

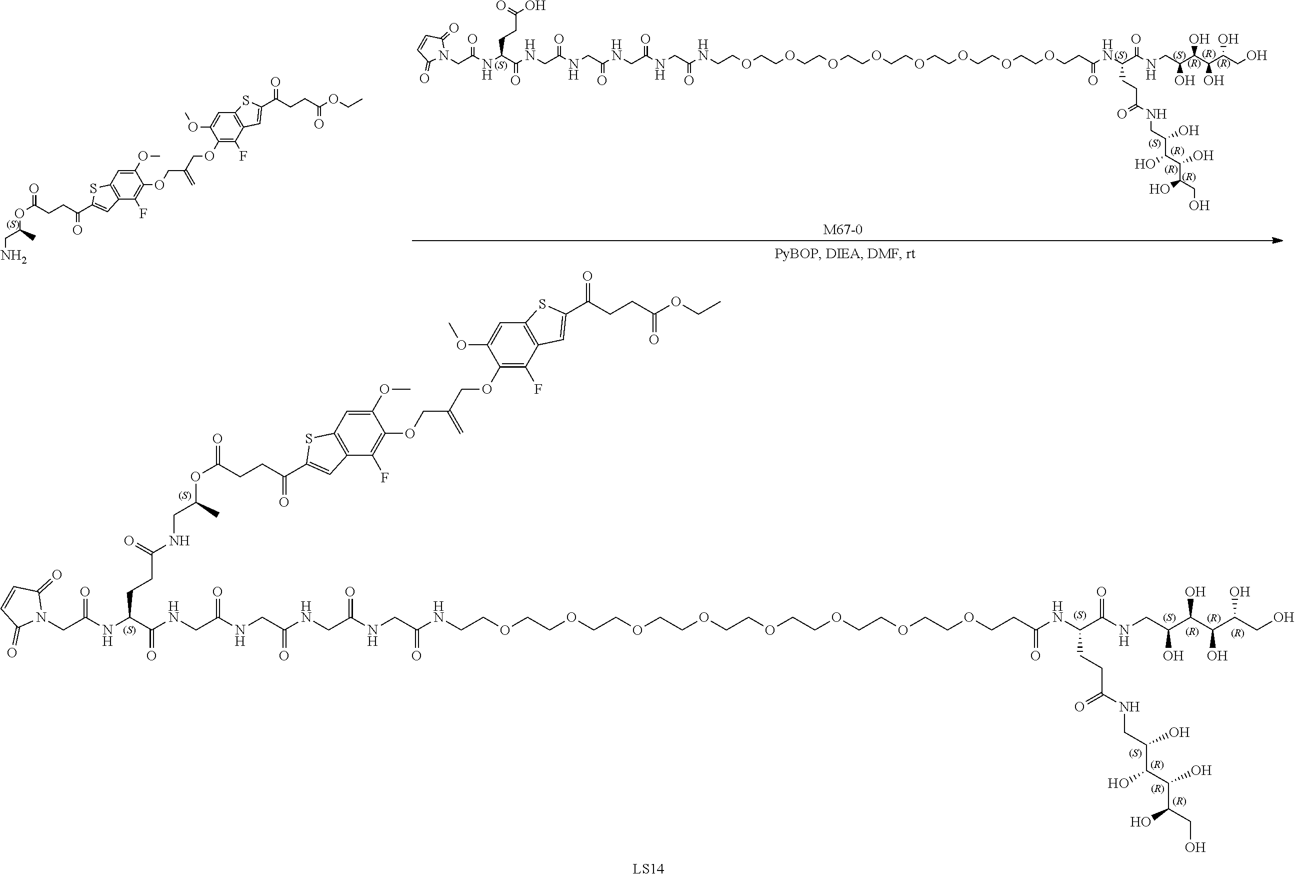

PyBOP (0.045 g, 86.4734 mol) was added to a solution of ethyl 4-[5-[2-[[2-[4-[(1S)-2-amino-1-methyl-ethoxy]-4-oxo-butanoyl]-4-fluoro-6-methoxy-benzothiophen-5-yl]oxymethyl]allyloxy]-4-fluoro-6-methoxy-benzothiophen-2-yl]-4-oxo-butanoate (0.031 g, 42.2462 mol), (4S,47S,53S,54R,55R,56R)-4-(2-(2,5-dioxo-2,5-dihydro-1H-pyrrol-1-yl)acetamido)-53,54,55,56,57-pentahydroxy-5,8,11,14,17,45,50-heptaoxo-47-(((2S,3R,4R,5R)-2,3,4,5,6-pentahydroxyhexyl)carbamoyl)-6,21,24,27,30,33,36,39,42-nonaoxa-9,12,15,18,46,51-hexaazaheptapentacontanoic acid (0.043 g, 30.9046 mol) and N,N-Diisopropylethylamine (26.7120 mg, 206.6814 mol) in DMF (1 mL) at 25° C. The reaction mixture was stirred for 2 h at 25° C. The residue was purified on C18 column MeCN/Water (0-65%). The purified fraction was concentrated and dried under vacuo. The product was purified by Prep-HPLC with the following conditions: (CXTH LC6000, HPLC-P4): Column, XB—C18,30*150 mm, 5 um; mobile phase, Water (0.1% TFA) and MeCN-20-48-50% B(2-30-33 min); Detector, uv 320 nm). The purified fraction was concentrated and dried by lyohpilization at 25° C.

There was ethyl 4-[5-[2-[[2-[4-[(1S)-2-[[(4S)-4-[[2-(2,5-dioxopyrrol-1-yl)acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]eth yl]amino]ethyl]amino]pentanoyl]amino]-1-methyl-ethoxy]-4-oxo-butanoyl]-4-fluoro-6-methoxy-benzothiophen-5-yl]oxymethyl]allyloxy]-4-fluoro-6-methoxy-benzothiophen-2-yl]-4-oxo-butanoate (17.2 mg, 8.1626 mol, 26.4124% yield) obtained as a white solid. LCMS: (ESI, m/z): [M+2H]²=1052.602.

### Example IV-15 Synthesis of Linker-STING Agonist Compound LS15

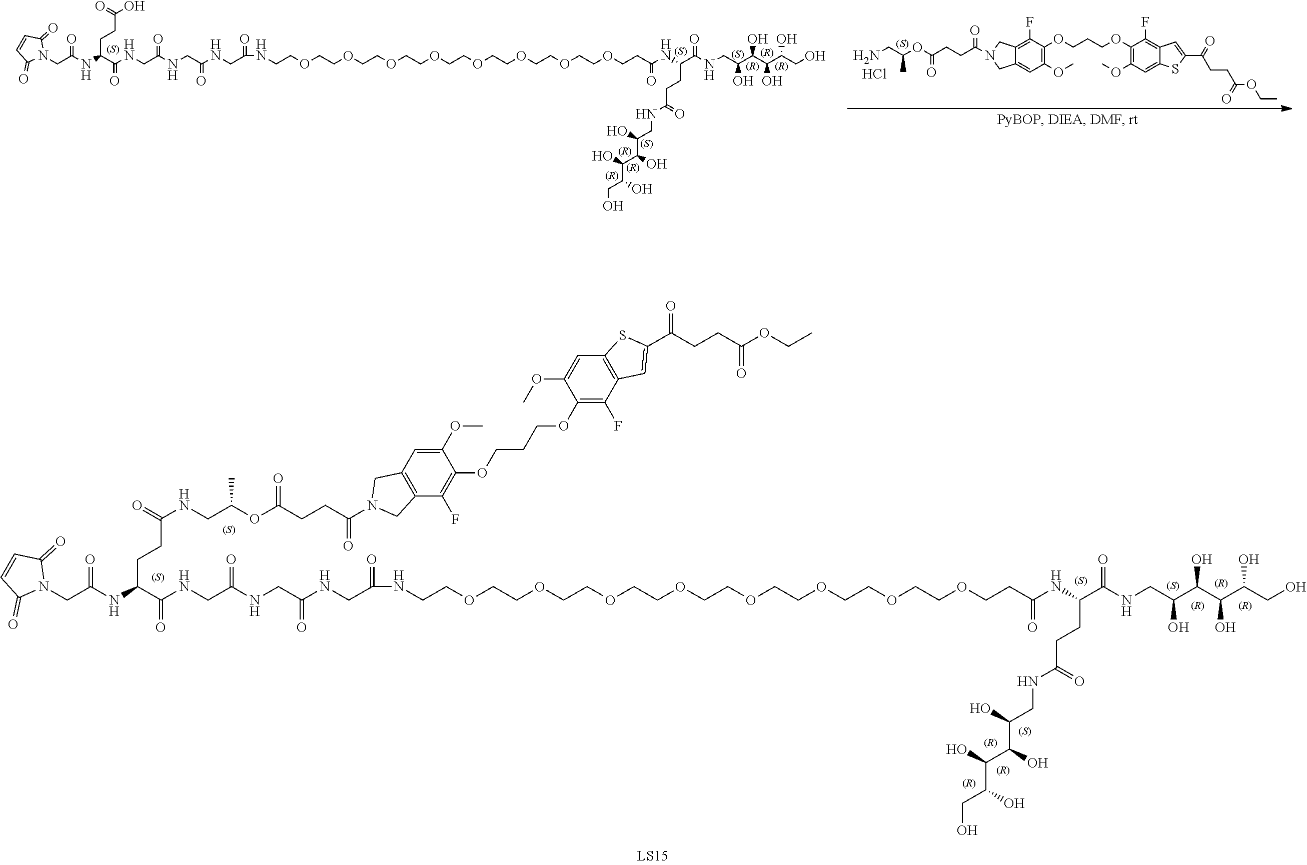

PyBOP (0.0589 g, 113.1840 mol) was added to a solution of (4S,44S,50S,51R,52R,53R)-4-(2-(2,5-dioxo-2,5-dihydro-1H-pyrrol-1-yl)acetamido)-50,51,52,53,54-pentahydroxy-5,8,11,14,42,47-hexaoxo-44-(((2S,3R,4R,5R)-2,3,4,5,6-pentahydroxyhexyl)carbamoyl)-18,21,24,27,30,33,36,39-octaoxa-6,9,12,15,43,48-hexaazatetrapentacontanoic acid (0.0526 g, 39.4206 mol), ethyl 4-[5-[3-[2-[4-[(1S)-2-amino-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.0314 g, 42.2492 mol, HCl salt), and DIEA (0.040 mL, 229.6460 μmol) in DMF (3 mL) at 21° C. The reaction mixture was stirred for 2 h at 21° C. The resulting reaction mixture was directly purified on C18 column ACN/0.1% TFA-H₂O(0-100%). The purified fraction was lyophilized overnight. The residue was further purified by prep-HPLC (the mobile phase: ACN/0.1% TFA-H₂O(0-50%) ). The purified fraction was lyophilized overnight. There was ethyl 4-[5-[3-[2-[4-[(1S)-2-[[(4S)-4-[[2-(2,5-dioxopyrrol-1-yl)acetyl]amino]-5-oxo-5-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]eth ylamino]ethyl]amino]ethyl]amino]ethyl]amino]pentanoyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.0315 g, 39.4983% yield) obtained as a white solid. LCMS: (ESI, m/z): [M+2H]²=1012.450.

### Example IV-16 Synthesis of Linker-STING Agonist Compound LS16

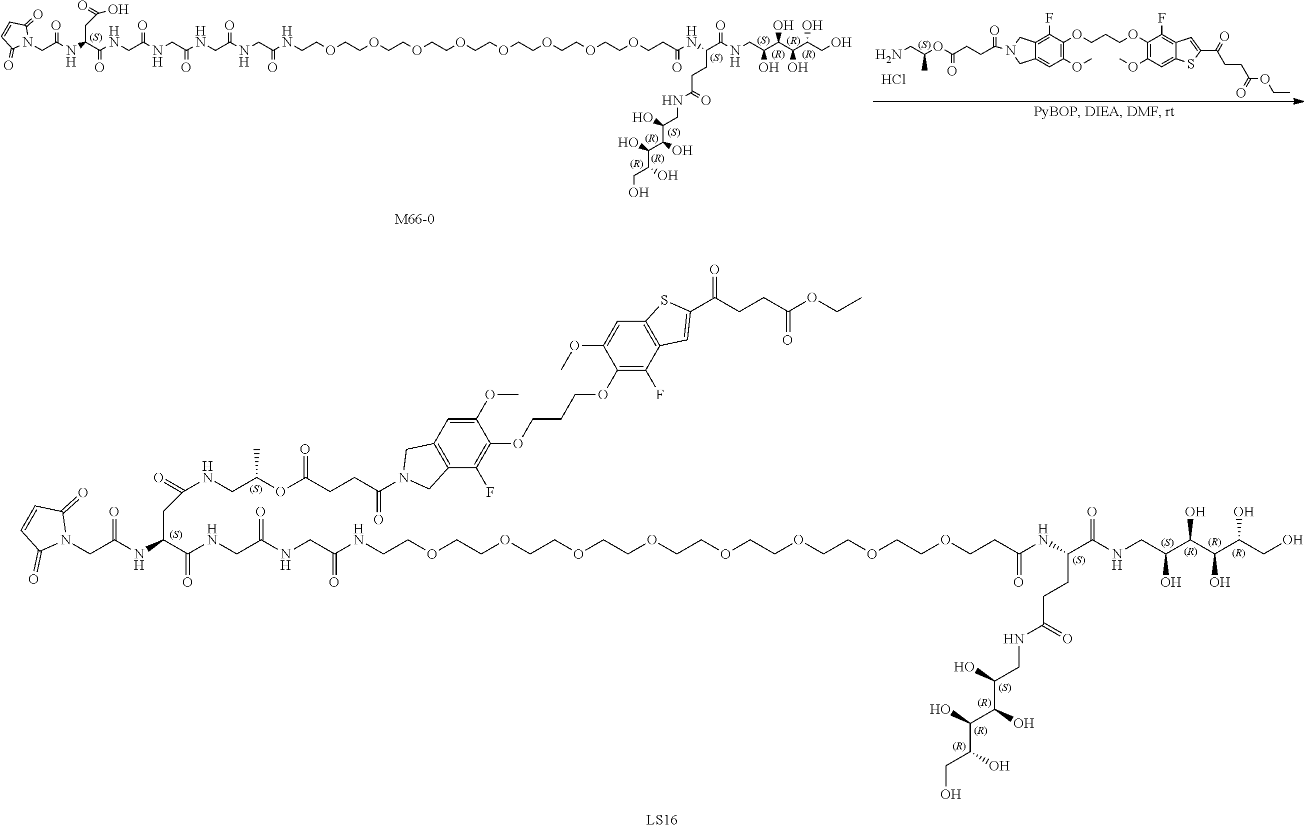

PyBOP (0.0653 g, 125.4825 mol) and DIEA (0.038 mL, 218.1637 mol) were added to a solution of (3S,46S,52S,53R,54R,55R)-3-(2-(2,5-dioxo-2,5-dihydro-1H-pyrrol-1-yl)acetamido) -52,53,54,55,56-pentahydroxy-4,7,10,13,16,44,49-heptaoxo-46-(((2S,3R,4R,5R)-2,3,4,5,6-pentahydroxyhexyl)carbamoyl)-20,23,26,29,32,35,38,41-octaoxa-5,8,11,14,17,45,50-heptaazahexapentacontanoic acid (M66-0, 0.0490 g, 35.5755 hmol) and ethyl 4-[5-[3-[2-[4-[(1S)-2-amino-1-methyl- ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.0293 g, 39.4236 μmol) in DMF (3 mL) at 10° C. The reaction mixture was stirred for 2 h at 10° C. The resulting reaction mixture was directly purified on C18 column ACN/0.1% TFA-H2O(0-100%). The purified fraction was lyophilized overnight. The residue was further purified by prep-HPLC (the mobile phase: ACN/0.1% TFA-H2O(0-50%) ). The purified fraction was lyophilized overnight. There was ethyl 4-[5-[3-[2-[4-[(1S)-2-[[(3S)-3-[[2-(2,5-dioxopyrrol-1-yl) acetyl]amino]-4-oxo-4-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2, 3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]butanoyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.0333 g, 45.3049% yield) obtained as a white solid. LCMS: (ESI, m/z): [M+2H]²⁺=1034.000.

### Example IV-17 Synthesis of Linker-STING Agonist Compound LS17

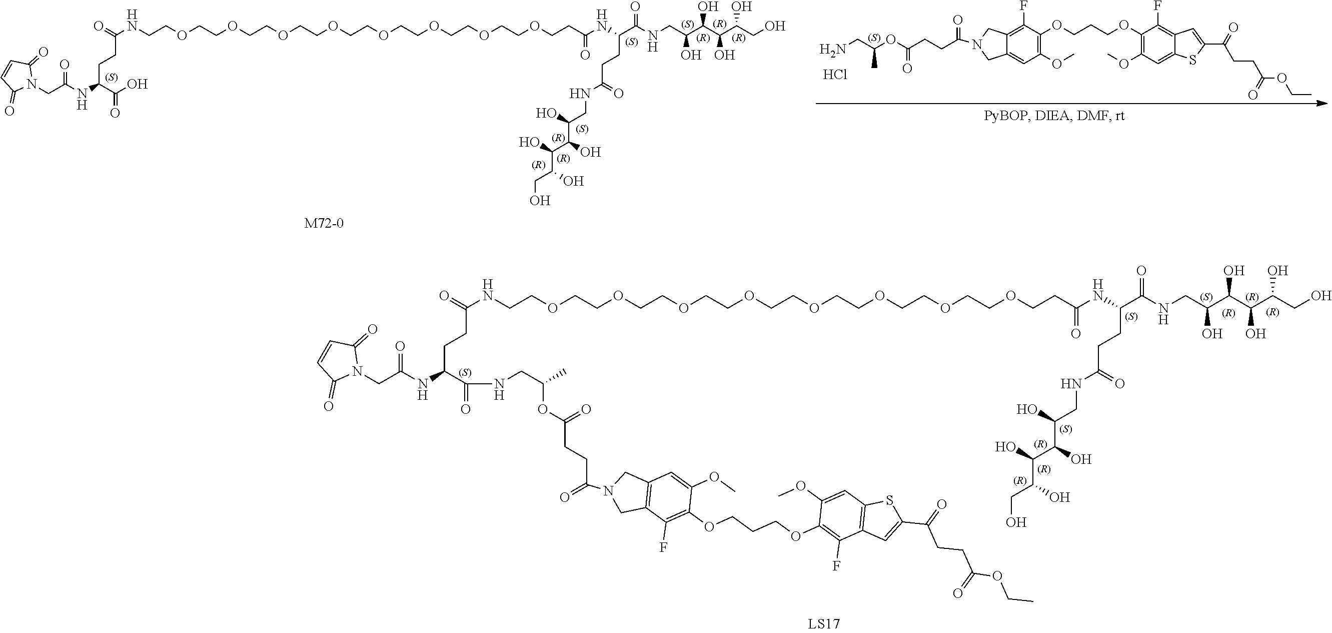

PyBOP (0.0587 g, 112.7997 mol) and DIEA (35 μL, 200.9402 mol) were added to a solution of (2S,35S,41S,42R,43R,44R)-2-(2-(2,5-dioxo-2,5-dihydro-1H-pyrrol-1-yl)acetamido)-41,42,43,44,45-pentahydroxy-5,33,38-trioxo-35-(((2S,3R,4R,5R)-2,3,4,5,6-pentahydroxyhexyl)carbamoyl)-9,12,15,18,21,24,27,30-octaoxa-6,34,39-triazapentatetracontanoic acid(M72-0, 0.0436 g, 37.4838 mol) and ethyl-4-[5-[3-[2-[4-[(1S)-2-amino-1-methyl-ethoxy]-4-oxo-butanoyl]-4-fluoro-6- methoxy-isoindolin-5-yl]oxypropoxy]-4-fluoro-6-methoxy-benzothiophen-2-yl]-4-oxo-butanoate (0.0288 g, 38.7508 mol, HCl salt) in DMF (3 mL) at 12° C. The reaction mixture was stirred for 2 h at 12° C. The resulting reaction mixture was directly purified on C18 column ACN/0.1% TFA-H₂O(0-100%). The purified fraction was lyophilized overnight. The residue was further purified by prep-HPLC (the mobile phase: ACN/0.1% TFA-H₂O(0-50%) ).

The purified fraction was lyophilized overnight. There was ethyl 4-[5-[3-[2-[4-[(1S)-2-[[(2S)-2-[[2-(2,5-dioxopyrrol-1-yl)acetyl]amino]-5-oxo-5-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]pentanoyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.0373 g, 53.7336% yield) obtained as a white solid. LCMS: (ESI, m/z): [M+2H]²⁺=926.800.

### Example IV-18 Synthesis of Linker-STING Agonist Compound LS18

### Step a:

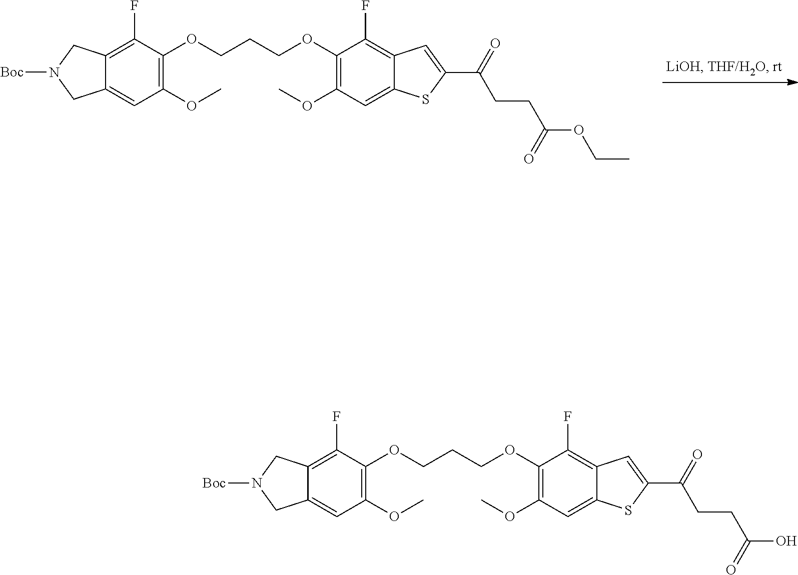 [SMILES: CCOC(=O)CCC(=O)c1cc2c(F)c(OCCCOc3c(OC)cc4c(c3F)CN(C(=O)OC(C)(C)C)C4)c(OC)cc2s1 ; COc1cc2c(c(F)c1OCCCOc1c(OC)cc3sc(C(=O)CCC(=O)O)cc3c1F)CN(C(=O)OC(C)(C)C)C2]

LiOH (0.09 g, 3.7581 mmol) was added to a mixture of tert-butyl 5-(3-((2-(4-ethoxy-4-oxobutanoyl)-4-fluoro-6-methoxybenzo[b]thiophen-5-yl)oxy)propoxy)-4-fluoro-6-methoxyisoindoline-2-carboxylate (0.35 g, 538.7120 mol) in THF (10 mL) and water (5 mL) at 20° C. The reaction mixture was stirred for 2 h at 20° C. The reaction mixture was adjusted to pH=6 with HCl (1 mol/L) and then extracted with EA (3×100 mL), The organic layer was separated and washed with brine (60 mL), dried over Na₂SO₄, filtered and evaporated under reduced pressure. There was 4-[5-[3-(2-tert-butoxycarbonyl-4-fluoro-6-methoxy-isoindolin-5-yl)oxypropoxy]-4-fluoro-6-methoxy-benzothiophen-2-yl]-4-oxo-butanoic acid (0.31 g, 498.6771 mol, 92.5684% yield) obtained as an off-white solid, which was used directly to the next step. LCMS: (ESI, m/z): [M+H]⁺=622.180. ¹H NMR (400 MHz, DMSO-d₆) δ 12.22 (s, 1H), 8.30 (s, 1H), 7.56 (s, 1H), 6.88 (s, 0.5H), 6.85 (s, 0.5H), 4.52 (s, 2H), 4.49 (s, 2H), 4.25 (t, J=6.1 Hz, 2H), 4.18 (t, J=6.1 Hz, 2H), 3.88 (d, J=1.9 Hz, 3H), 3.76 (d, J=4.4 Hz, 3H), 3.33-3.27 (m, 2H), 2.60 (t, J=6.4 Hz, 2H), 2.08-2.01 (m, 2H), 1.45 (s, 9H).

### Step b:

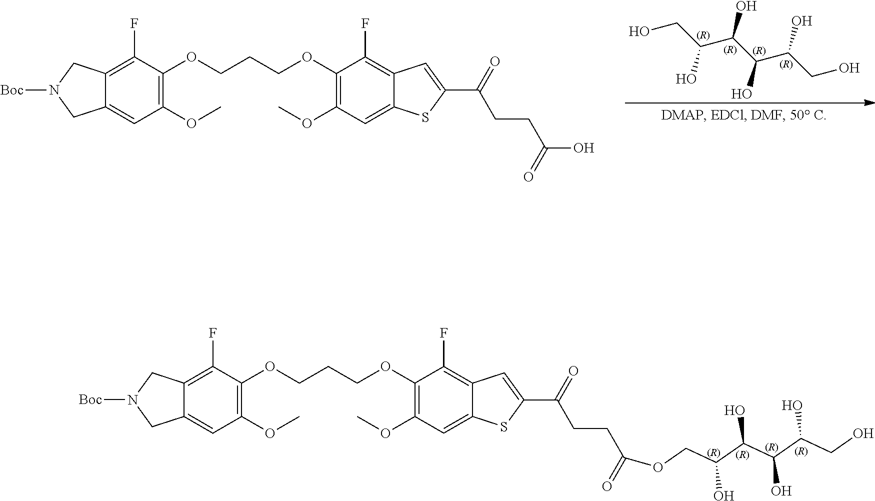

(2R,3R,4R,5R)-hexane-1,2,3,4,5,6-hexaol (2.12 g, 11.6374 mmol) was added to a mixture of 4-[5-[3-(2-tert-butoxycarbonyl-4-fluoro-6-methoxy-isoindolin-5-yl)oxypropoxy]-4-fluoro-6-methoxy-benzothiophen-2-yl]-4-oxo-butanoic acid (0.89 g, 1.4317 mmol), EDCI (0.91 g, 4.7470 mmol) and N-(4-pyridyl)dimethylamine (0.35 g, 2.8649 mmol) in DMF (70 mL) at 20° C. The reaction mixture was heated to 50° C. and stirred for 12 h. The reaction mixture was diluted with H₂O (200 mL), extracted with EA (4×200 mL) and brine (100 mL). The organics were dried over Na₂SO₄, filtered and evaporated under reduced pressure. The residue was purified on C18 column ACN/H₂O (0.1% TFA)(0-60%). The purified fraction was concentrated and dried under vacuo. There was tert-butyl 4-fluoro-5-[3-[4-fluoro-6-methoxy-2-[4-oxo-4-[(2R,3R,4R,5R)-2,3,4,5,6-pentahydroxyhexoxy]butanoyl]benzothiophen-5-yl]oxypropoxy]-6-methoxy-isoindoline-2-carboxylate (1.37 g, 75% purity, 1.7434 mmol, 91.3317% yield) obtained as a brown solid. LCMS: (ESI, m/z): [M+H]⁺=786.250.

### Step c:

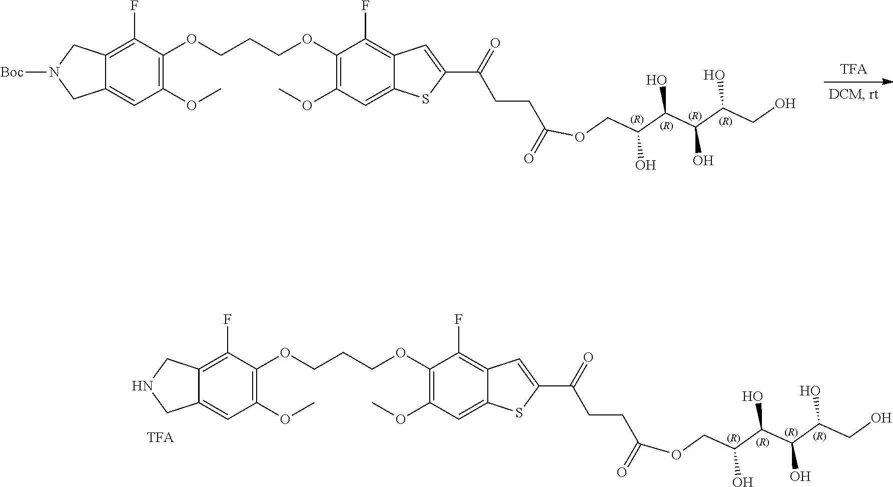

LiOH (0.043 g, 1.7955 mmol) was added to a solution of ethyl 4-[5-[3-[2-(4-tert-butoxy-4-oxo-butanoyl)-4-chloro-6-methoxy-isoindolin-5-yl]oxypropoxy]-4-fluoro-6-methoxy-benzothiophen-2-yl]-4-oxo-butanoate (0.520 g, 75% purity, 564.6231 mol) in water (3 mL) and THF (6 mL) at 20° C. under open-air atmosphere. The reaction mixture was stirred for 2 h at 20° C. The reaction mixture was adjusted to pH=6 with HCl (1 μmol/L). The mixture was purified on C18 column ACN/H₂O (0.1% TFA)(0-55%). The purified fraction was concentrated and dried under vacuo. There was (2R,3R,4R,5R)-2,3,4,5,6-pentahydroxyhexyl-4-(4-fluoro-5-(3-((4-fluoro-6-methoxyisoindolin-5-yl)oxy)propoxy)-6-methoxybenzo[b]thiophen-2-yl)-4-oxobutanoate compound with 2,2,2-trifluoroacetaldehyde (1:1) (0.41 g, 528.8090 mol, 93.6570% yield) obtained as a white solid. LCMS: (ESI, m/z): [M+H]⁺=686.200.

### Step d:

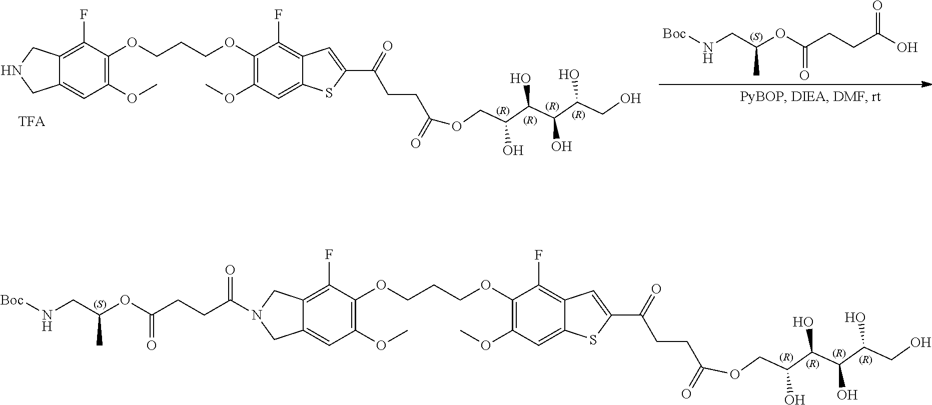

(2R,3R,4R,5R)-2,3,4,5,6-pentahydroxyhexyl 4-(4-fluoro-5-(3-((4-fluoro-6-methoxyisoindolin-5-yl)oxy)propoxy)-6-methoxybenzo[b]thiophen-2-yl)-4-oxobutanoate compound with 2,2,2-trifluoroacetaldehyde (1:1) (0.159 g, 231.8848 mol) and N,N-diisopropylethylamine (0.238 g, 1.8415 mmol) was added to a solution of (S)-4-((1-((tert-butoxycarbonyl)amino)propan-2-yl)oxy)-4-oxobutanoic acid (0.143 g, 519.4383 mol) and PyBOP (0.348 g, 668.7274 mol) in DMF (5 mL) at 20° C. under N₂ atmosphere. The reaction mixture was stirred for 4 h at 20° C. The reaction mixture was purified on C18 column ACN/H₂O (0.1% TFA)(0-35%). The purified fraction was concentrated and dried under vacuo. There was [(2R,3R,4R,5R)-2,3,4,5,6-pentahydroxyhexyl]-4-[5-[3-[2-[4-[(1S)-2-(tert-butoxycarbonylamino)-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.20 g, 212.0963 mol, 91.4663% yield) obtained as a yellow solid.

LCMS: (ESI, m/z): [M+H]⁺=943.330.

### Step e:

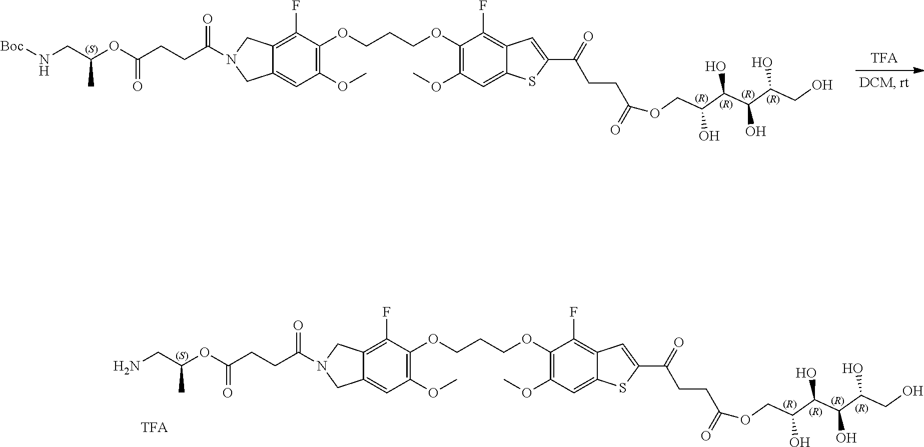

[(2R,3R,4R,5R)-2,3,4,5,6-pentahydroxyhexyl]-4-[5-[3-[2-[4-[(1S)-2-(tert-butoxycarbonylamino)-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.22 g, 233.3060 mol) was added to a solution of trifluoroacetic acid, TFA (26.6023 mg, 233.3060 mol) and DCM (2 mL) at 20° C. under N₂ atmosphere. The reaction mixture was stirred for 1 h at 20° C. The reaction mixture was evaporated under reduced pressure. The residue was purified on C18 column ACN/H₂O (0.1% TFA)(0-40%). The purified fraction was concentrated and dried under vacuo. There was (S)-1-aminopropan-2-yl 4-(4-fluoro-5-(3-((4-fluoro-6- methoxy-2-(4-oxo-4-(((2R,3R,4R,5R)-2,3,4,5,6-pentahydroxyhexyl)oxy)butanoyl)benzo[b]thiophen-5-yl)oxy)propoxy)-6-methoxyisoindolin-2-yl)-4-oxobutanoate compound with 2,2,2-trifluoro-113-ethan-1-one (1:1) (0.0662 g, 78.5429 mol, 33.6652% yield) obtained as a yellow solid. LCMS: (ESI, m/z): [M+H]⁺=843.270.

### Step f:

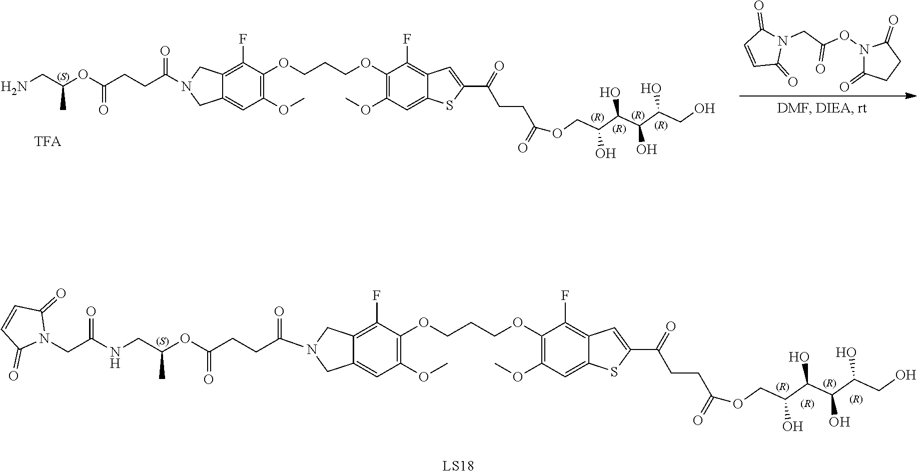

N,N-diisopropylethylamine (0.0076 g, 58.8042 mol) was added to a solution of (S)-1-aminopropan-2-yl-4-(4-fluoro-5-(3-((4-fluoro-6-methoxy-2-(4-oxo-4-(((2R,3R,4R,5R)-2,3,4,5,6-pentahydroxyhexyl)oxy)butanoyl)benzo[b]thiophen-5-yl)oxy)propoxy)-6-methoxyisoindolin-2-yl)-4-oxobutanoate compound with 2,2,2-trifluoro-113-ethan-1-one (1:1) (0.0150 g, 17.7967 mol) and N-Succinimidyl maleimidoacetate (0.0104 g, 41.2403 mol) in DMF (3 mL) at 20° C. under N₂ atmosphere. The reaction mixture was stirred for 1.5 h at 20° C. The reaction mixture was purified on C-18 column ACN/H₂O (0.1% TFA)(0-35%). The purified fraction was concentrated and dried under vacuo. The residue was purified on Prep-HPLC. The purified fraction was concentrated and dried by lyophilization. There was [(2R,3R,4R,5R)-2,3,4,5,6-pentahydroxyhexyl]-4-[5-[3-[2-[4-[(1S)-2-[[2-(2,5-dioxopyrrol-1-yl)acetyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.011 g, 11.2251 mol, 63.0741% yield) obtained as a white solid.

LCMS: (ESI, m/z): [M+H]⁺=980.30.

### Example IV-19 Synthesis of Linker-STING Agonist Compound LS19

### Step a:

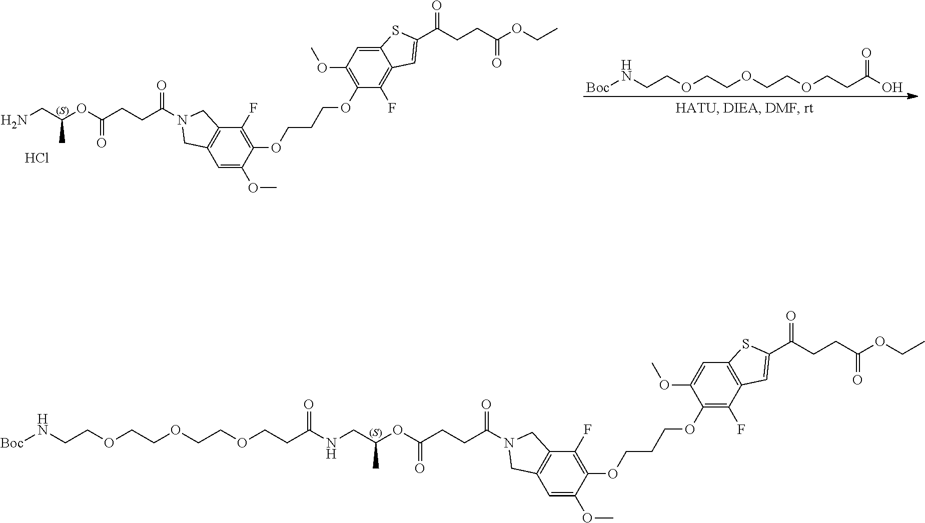

(S)-1-aminopropan-2-yl-4-(5-(3-((2-(4-ethoxy-4-oxobutanoyl)-4-fluoro-6- methoxybenzo[b]thiophen-5-yl)oxy)propoxy)-4-fluoro-6-methoxyisoindolin-2-yl)-4-oxobutanoate hydrochloride (0.044 g, 59.2026 μmol) and N,N-Diisopropylethylamine (0.048 g, 371.3951 μmol) was added to a solution of 2,2-dimethyl-4-oxo-3,8,11,14-tetraoxa-5-azaheptadecan-17-oic acid (0.047 g, 146.2509 μmol) and HATU (0.047 g, 123.6096 μmol) in DMF (3 mL) at 20° C. under open-air atmosphere. The reaction mixture was stirred for 2 h at 20° C. The reaction mixture was purified on C18 column ACN/H₂O (0.1% TFA)(0-65%). The purified fraction was concentrated and dried under vacuo. There was ethyl 4-[5-[3-[2-[4-[(1S)-2-[3-[2-[2-[2-(tert-butoxycarbonylamino)ethoxy]ethoxy]ethoxy]propanoylamino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.047 g, 46.5301 μmol, 78.5946% yield) obtained as a white solid. LCMS: (ESI, m/z): [M+H]⁺=1010.410.

### Step b:

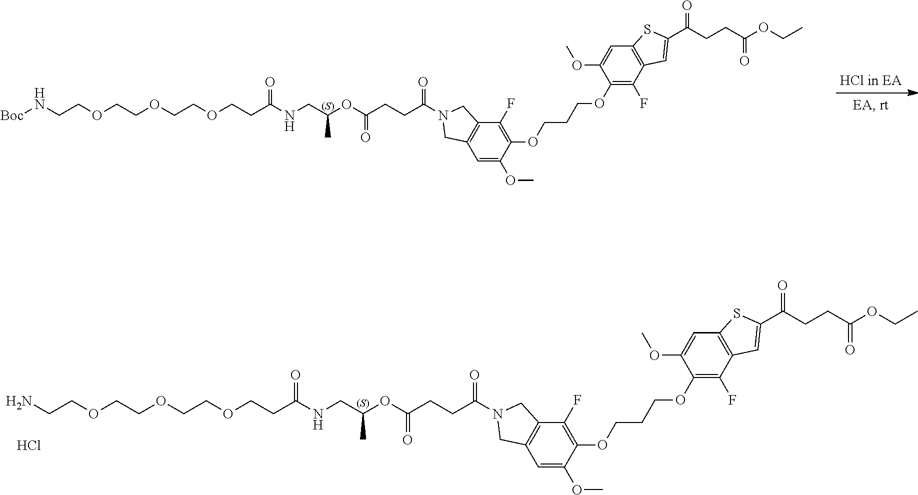

HCl in EA (5 mL, 20 mmol) was added to a solution of ethyl 4-[5-[3-[2-[4-[(1S)-2-[3-[2-[2-[2-(tert-butoxycarbonylamino)ethoxy]ethoxy]ethoxy]propanoylamino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.046 g, 45.5401 μmol) in EA (2 mL) at 20° C. under open-air atmosphere. The reaction mixture was stirred for 1 h at 20° C. The reaction mixture was evaporated under reduced pressure. The residue was purified on C-18 column ACN/H₂O (0.1% TFA)(0-50%). The purified fraction was dried by lyophilization. There was (S)-1-amino-12-oxo-3,6,9-trioxa-13-azahexadecan-15-yl-4-(5-(3-((2-(4-ethoxy-4-oxobutanoyl)-4-fluoro-6-methoxybenzo[b]thiophen-5-yl)oxy)propoxy)-4-fluoro-6-methoxyisoindolin-2-yl)-4-oxobutanoate hydrochloride (0.0349 g, 38.3523 mol, 84.2167% yield) obtained as a white solid. LCMS: (ESI, m/z): [M+H]⁺=910.350.

### Step c:

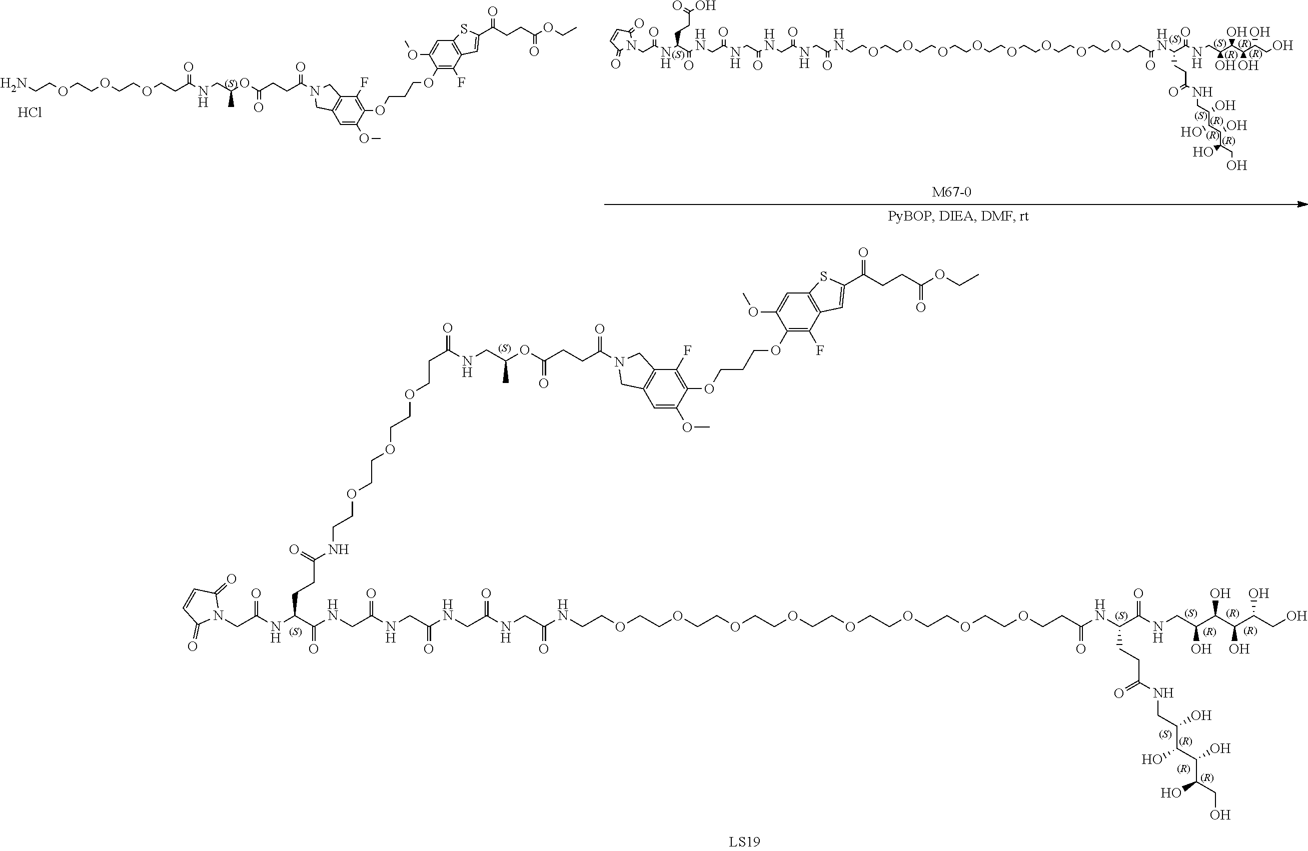

(S)-1-amino-12-oxo-3,6,9-trioxa-13-azahexadecan-15-yl-4-(5-(3-((2-(4-ethoxy-4-oxobutanoyl)-4-fluoro-6-methoxybenzo[b]thiophen-5-yl)oxy)propoxy)-4-fluoro-6-methoxyisoindolin-2-yl)-4-oxobutanoate hydrochloride (0.036 g, 39.5611 μmol) and N,N-diisopropylethylamine (0.041 g, 317.2334 μmol) was added to a solution of (4S,47S,53S,54R,55R,56R)-4-(2-(2,5-dioxo-2,5-dihydro-1H-pyrrol-1-yl)acetamido)-53,54,55,56,57-pentahydroxy-5,8,11,14,17,45,50-heptaoxo-47-(((2S,3R,4R,5R)-2,3,4,5,6-pentahydroxyhexyl) carbamoyl)-21,24,27,30,33,36,39,42-octaoxa-6,9,12,15,18,46,51-heptaazaheptapentacontanoic acid (0.041 g, 29.4672 μmol) and PyBOP (0.050 g, 96.0815 μmol) in DMF (3 mL) at 20° C. under N₂ atmosphere. The reaction mixture was stirred for 2 h at 20° C. The reaction mixture was purified on C18 column ACN/H₂O (0.1% TFA)(0-35%). The purified fraction was concentrated and dried under vacuo. The residue was purified on HPLC. The purified fraction was dried by lyophilization. There was ethyl 4-[5-[3-[2-[4-[(1S)-2-[3-[2-[2-[2-[[(4S)-4-[[2-(2,5-dioxopyrrol-1-yl)acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]pentanoyl]amino]ethoxy]ethoxy]ethoxy]propanoylamino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.0176 g, 7.7080 μmol, 19.4837% yield) obtained as a white solid. LCMS: (ESI, m/z): [M+2H]²⁺=1164.700.

### Example IV-20 Synthesis of Linker-STING Agonist Compound LS20 G-87C

### Step a:

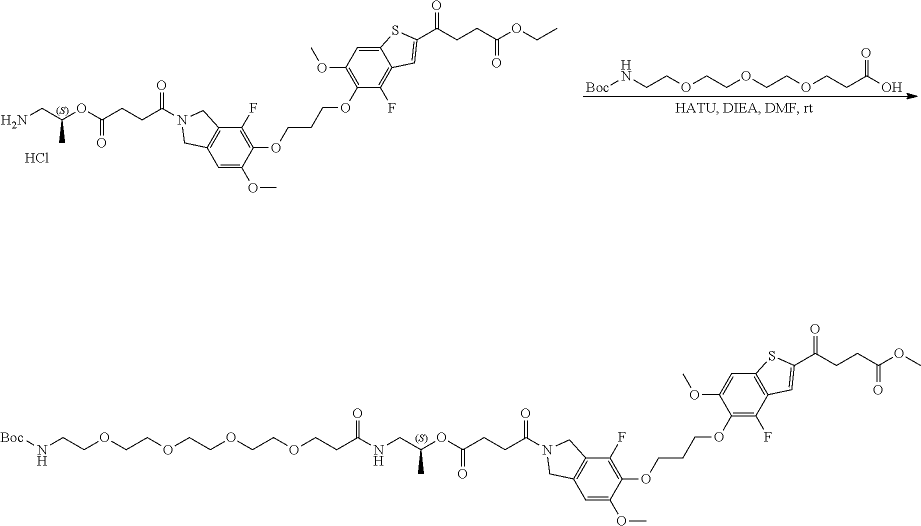

(S)-1-aminopropan-2-yl-4-(5-(3-((2-(4-ethoxy-4-oxobutanoyl)-4-fluoro-6- methoxybenzo[b]thiophen-5-yl)oxy)propoxy)-4-fluoro-6-methoxyisoindolin-2-yl)-4-oxobutanoate hydrochloride (0.045 g, 60.5482 μmol) and N,N-Diisopropylethylamine (0.047 g, 363.6577 μmol) was added to a solution of 2,2-dimethyl-4-oxo-3,8,11,14,17-pentaoxa-5-azaicosan-20-oic acid (0.049 g, 134.0930 μmol) and HATU (0.051 g, 134.1296 μmol) in DMF (3 mL) at 20° C. under open-air atmosphere. The reaction mixture was stirred for 2 h at 20° C. The reaction mixture was purified on C18 column ACN/H₂O (0.1% TFA)(0-65%). The purified fraction was concentrated and dried under vacuo. There was ethyl 4-[5-[3-[2-[4-[(1S)-2-[[2-[2-[2-[2-[2-(tert-butoxycarbonylamino)ethoxy]ethoxy]ethoxy]ethoxy]acetyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.046 g, 44.2254 mol, 73.0418% yield) obtained as a white solid. LCMS: (ESI, m/z): [M+H]⁺=1054.430.

### Step b:

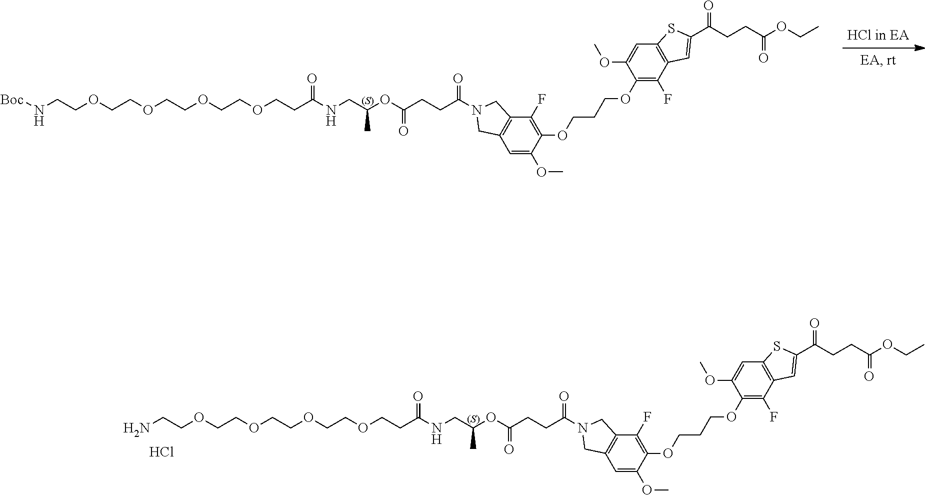

HCl in EA (5 mL, 20 mmol) was added to a solution of ethyl 4-[5-[3-[2-[4-[(1S)-2-[[2-[2-[2-[2-[2-(tert- butoxycarbonylamino)ethoxy]ethoxy]ethoxy]ethoxy]acetyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.045 g, 43.2640 mol) in EA (2 mL) at 20° C. under open-air atmosphere. The reaction mixture was stirred for 1 h at 20° C. The reaction mixture was evaporated under reduced pressure. The residue was purified on C18 column ACN/H₂O (0.1% TFA)(0-50%). The purified fraction was dried by lyophilization. There was (S)-1-amino-15-oxo-3,6,9,12-tetraoxa- 16-azanonadecan-18-yl-4-(5-(3-((2-(4-ethoxy-4-oxobutanoyl)-4-fluoro-6-methoxybenzo[b]thiophen-5-yl)oxy)propoxy)-4-fluoro-6-methoxyisoindolin-2-yl)-4-oxobutanoate hydrochloride (0.0316 g, 33.1224 mol, 76.5588% yield) obtained as a white solid. LCMS: (ESI, m/z): [M+H]⁺=954.380.

### Step c:

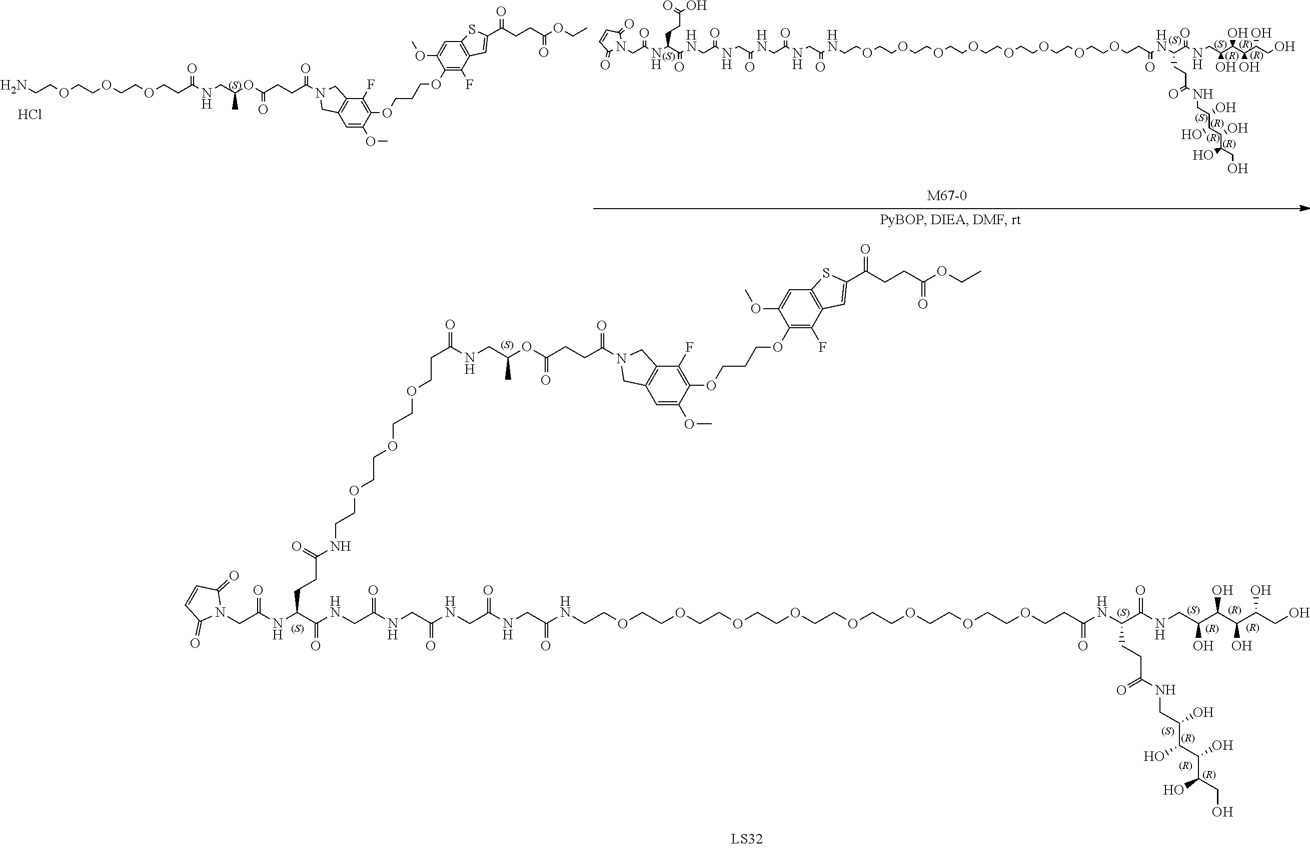

(S)-1-amino-15-oxo-3,6,9,12-tetraoxa-16-azanonadecan-18-yl 4-(5-(3-((2-(4-ethoxy-4-oxobutanoyl)-4-fluoro-6-methoxybenzo[b]thiophen-5-yl)oxy)propoxy)-4-fluoro-6-methoxyisoindolin-2-yl)-4-oxobutanoate hydrochloride (0.030 g, 31.4453 μmol) and N,N-Diisopropylethylamine (0.043 g, 332.7082 μmol) was added to a solution of (4S,47S,53S,54R,55R,56R)-4-(2-(2,5-dioxo-2,5-dihydro-1H-pyrrol-1-yl)acetamido)-53,54,55,56,57-pentahydroxy-5,8,11,14,17,45,50-heptaoxo-47-(((2S,3R,4R,5R)-2,3,4,5,6-pentahydroxyhexyl) carbamoyl)-21,24,27,30,33,36,39,42-octaoxa-6,9,12,15,18,46,51-heptaazaheptapentacontanoic acid (0.032 g, 22.9988 μmol)) and PyBOP (0.049 g, 94.1599 μmol) in DMF (3 mL) at 20° C. under N₂ atmosphere. The reaction mixture was stirred for 2 h at 20° C. The reaction mixture was purified on C18 column ACN/H₂O (0.1% TFA)(0-35%). The purified fraction was concentrated and dried under vacuo. The residue was purified on HPLC. The purified fraction was dried by lyophilization. There was (2S,24S,67S,73S,74R,75R,76R)-24-(2-(2,5-dioxo-2,5-dihydro-1H-pyrrol-1-yl)acetamido)-73,74,75,76,77-pentahydroxy-5,21,25,28,31,34,37,65,70-nonaoxo-67-(((2S,3R,4R,5R)-2,3,4,5,6-pentahydroxyhexyl)carbamoyl)-8,11,14,17,41,44,47,50,53,56,59,62-dodecaoxa-4,20,26,29,32,35,38,66,71-nonaazaheptaheptacontan-2-yl 4-(5-(3-((2-(4-ethoxy-4-oxobutanoyl)-4-fluoro-6-methoxybenzo[b]thiophen-5-yl)oxy)propoxy)-4-fluoro-6-methoxyisoindolin-2-yl)-4-oxobutanoate (0.0188 g, 8.0777 mol, 25.6880% yield) obtained as a white solid. LCMS: (ESI, m/z): [M+2H]²⁺=1142.750.

### Example IV-21 Synthesis of Linker-STING Agonist Compound LS21

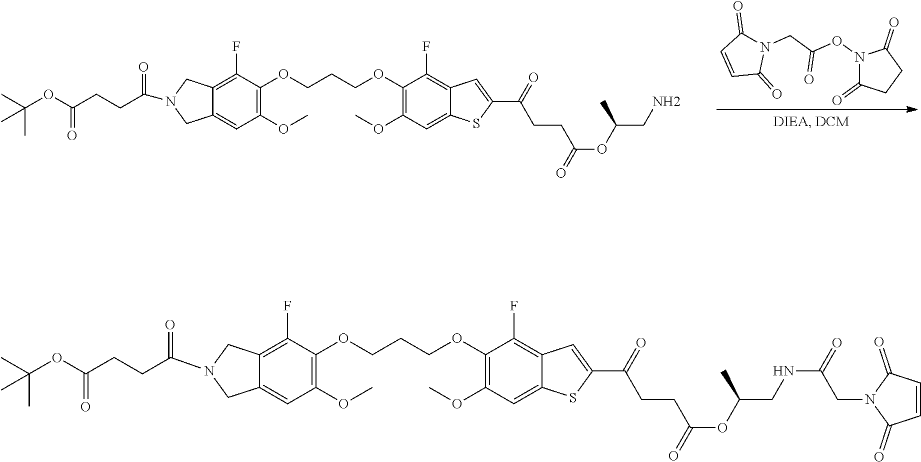 [SMILES: COc1cc2c(c(F)c1OCCCOc1c(OC)cc3sc(C(=O)CCC(=O)O[C@@H](C)CN)cc3c1F)CN(C(=O)CCC(=O)OC(C)(C)C)C2 ; O=C(CN1C(=O)C=CC1=O)ON1C(=O)CCC1=O ; COc1cc2c(c(F)c1OCCCOc1c(OC)cc3sc(C(=O)CCC(=O)O[C@@H](C)CNC(=O)CN4C(=O)C=CC4=O)cc3c1F)CN(C(=O)CCC(=O)OC(C)(C)C)C2]

(S)-1-aminopropan-2-yl-4-(5-(3-((2-(4-(tert-butoxy)-4-oxobutanoyl)-4-fluoro-6-methoxyisoindolin-5-yl)oxy)propoxy)-4-fluoro-6-methoxybenzo[b]thiophen-2-yl)-4-oxobutanoate (0.0412 g, 56.0695 mol) was added to a solution of DIEA (0.014 mL, 80.3761 mol) and N-succinimidyl maleimidoacetate (0.0220 g, 87.2393 mol) in DCM (4 mL) at 12° C. The reaction mixture was stirred for 2 h at 12° C. The reaction mixture was concentrated under reduced pressure. The crude was further purified by prep-HPLC (the mobile phase: ACN/0.1% TFA-H₂O(0-50%) ). The purified fraction was lyophilized overnight. There was [(1S)-2-[[2-(2,5-dioxopyrrol-1-yl)acetyl]amino]-1-methyl-ethyl]4-[5-[3-[2-(4-tert-butoxy-4- oxo-butanoyl)-4-fluoro-6-methoxy-isoindolin-5-yl]oxypropoxy]-4-fluoro-6-methoxy-benzothiophen-2-yl]-4-oxo-butanoate (0.044 g, 90.0040% yield) obtained as an off-white solid. LCMS: (ESI, m/z): [M+H]⁺=872.350.

### Step b:

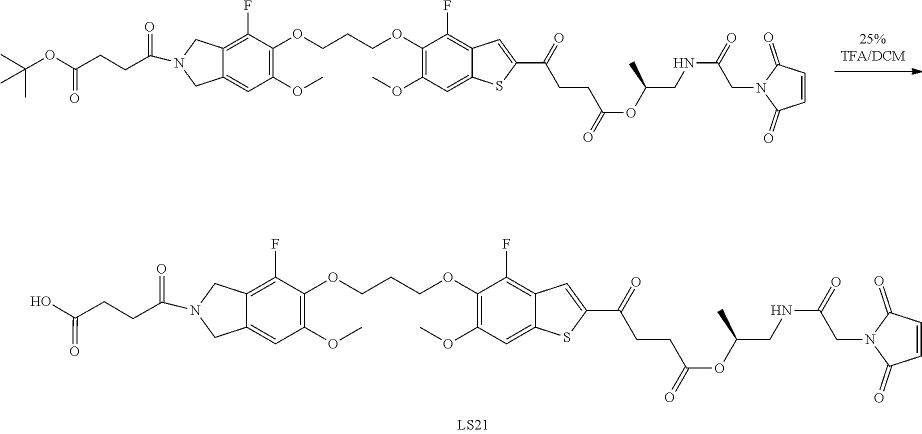 [SMILES: COc1cc2c(c(F)c1OCCCOc1c(OC)cc3sc(C(=O)CCC(=O)O[C@@H](C)CNC(=O)CN4C(=O)C=CC4=O)cc3c1F)CN(C(=O)CCC(=O)OC(C)(C)C)C2 ; COc1cc2c(c(F)c1OCCCOc1c(OC)cc3sc(C(=O)CCC(=O)O[C@@H](C)CNC(=O)CN4C(=O)C=CC4=O)cc3c1F)CN(C(=O)CCC(=O)O)C2]

[(1S)-2-[[2-(2,5-dioxopyrrol-1-yl)acetyl]amino]-1-methyl-ethyl]4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.042 g, 48.1709 mol) was dissolved in 25% TFA (3 mL) in DCM at 14° C. The reaction mixture was stirred for 1.0 h at 14° C. The reaction mixture was concentrated under reduced pressure. The residue was treated with diethyl ether to form the precipitate. The crude was further purified by prep-HPLC (the mobile phase: ACN/0.1% TFA-H₂O(0-50%)). The purified fraction was lyophilized overnight. There was 4-[5-[3-[2-[4-[(1S)-2-[[2-(2,5-dioxopyrrol-1-yl)acetyl]amino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (0.0132 g, 33.5901% yield) obtained as a white solid. LCMS: (ESI, m/z): [M+H]⁺=816.250. ¹H NMR (400 MHz, DMSO-d₆) δ 12.09 (s, 1H), 8.31 (s, 1H), 8.25 (t, J=5.7 Hz, 1H), 7.56 (s, 1H), 7.08 (s, 2H), 6.90 (s, 0.5H), 6.87 (s, 0.5H), 4.86-4.80 (M, 1H), 4.78 (s, 2H), 4.56 (d, J=6.3 Hz, 2H), 4.25 (t, J=5.8 Hz, 2H), 4.19 (t, J=5.8 Hz, 2H), 4.04 (s, 2H), 3.88 (s, 3H), 3.77 (s, 3H), 3.43-3.34 (m, 4H), 3.25-3.16 (m, 2H), 3.68-2.62 (m, 2H), 2.59-2.54 (m, 2H), 2.11-2.00 (m, 2H), 1.11 (d, J=6.3 Hz, 3H).

### Example IV-22 Synthesis of Linker-STING Agonist Compound LS22

### Step a:

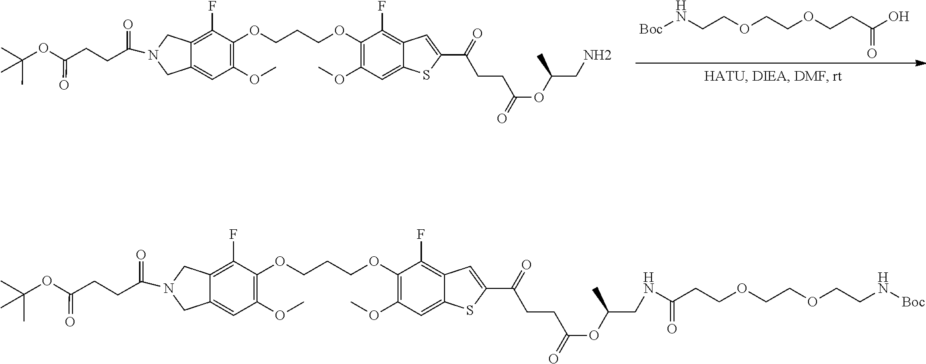 [SMILES: COc1cc2c(c(F)c1OCCCOc1c(OC)cc3sc(C(=O)CCC(=O)O[C@@H](C)CN)cc3c1F)CN(C(=O)CCC(=O)OC(C)(C)C)C2 ; CC(C)(C)OC(=O)NCCOCCOCCC(=O)O ; COc1cc2c(c(F)c1OCCCOc1c(OC)cc3sc(C(=O)CCC(=O)O[C@@H](C)CNC(=O)CCOCCOCCNC(=O)OC(C)(C)C)cc3c1F)CN(C(=O)CCC(=O)OC(C)(C)C)C2]

HATU (123.0 mg, 323.4890 mol) was added to a solution of [(1S)-2-amino-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (119.6 mg, 162.7649 mol), DIEA (74.2000 mg, 574.1150 mol) 2,2-dimethyl-4-oxo-3,8,11-trioxa-5-azatetradecan-14-oic acid (79.5 mg, 286.6795 mol) in DMF (2 mL) at 25° C. The reaction mixture was stirred for 2 h at 25° C. The residue was purified on C18 column MeCN/Water (0-75%). The purified fraction was concentrated and dried by lyohpilization at 25° C. There was [(1S)-2-[3-[2-[2-(tert-butoxycarbonylamino)ethoxy]ethoxy]propanoylamino]-1-methyl-ethyl]4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.094 g, 94.5579 mol, 58.0948% yield) obtained as a brown solid. LCMS: (ESI, m/z): [M+H]⁺=994.111.

### Step b:

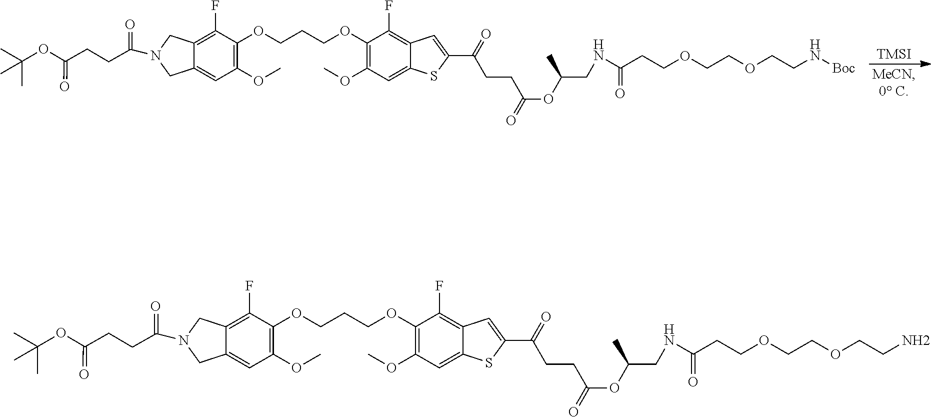 [SMILES: "TMSI" ; COc1cc2c(c(F)c1OCCCOc1c(OC)cc3sc(C(=O)CCC(=O)O[C@@H](C)CNC(=O)CCOCCOCCNC(=O)OC(C)(C)C)cc3c1F)CN(C(=O)CCC(=O)OC(C)(C)C)C2 ; "MeCN,0° C." ; COc1cc2c(c(F)c1OCCCOc1c(OC)cc3sc(C(=O)CCC(=O)O[C@@H](C)CNC(=O)CCOCCOCCN)cc3c1F)CN(C(=O)CCC(=O)OC(C)(C)C)C2]

TMSI (0.031 g, 154.9280 mol) in MeCN (2 ml) was added to a solution of tert-butyl (S)-4-(4-fluoro-5-(3-((4-fluoro-6-methoxy-2-(2,2,17-trimethyl-4,14,19-trioxo-3,8,11,18-tetraoxa-5,15-diazadocosan-22-oyl)benzo[b]thiophen-5-yl)oxy)propoxy)-6-methoxyisoindolin-2-yl)-4-oxobutanoate (0.093 g, 93.5520 mol) in MeCN (2 mL) at 0° C. The reaction mixture was stirred for 0.5 h at 0° C. The reaction mixture was stirred for 1 h at 25° C. The reaction mixture was quenched with H₂O (4 mL). The resulting reaction mixture was purified on C-18 column ACN/0.1% TFA-H₂O(0-75%). The purified fraction was concentrated and dried under vacuo.

There was [(1S)-2-[3-[2-(2-aminoethoxy)ethoxy]propanoylamino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.047 g, 52.5736 mol, 56.1972% yield) obtained as a colorless oil. LCMS: (ESI, m/z): [M+H]⁺=893.994.

### Step c:

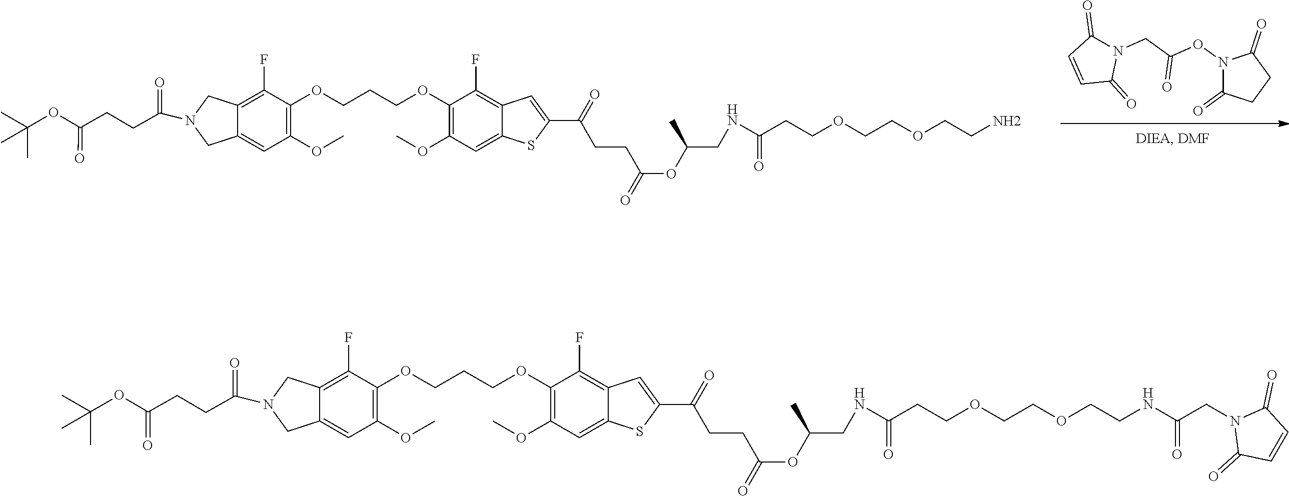 [SMILES: COc1cc2c(c(F)c1OCCCOc1c(OC)cc3sc(C(=O)CCC(=O)O[C@@H](C)CNC(=O)CCOCCOCCN)cc3c1F)CN(C(=O)CCC(=O)OC(C)(C)C)C2 ; O=C(CN1C(=O)C=CC1=O)ON1C(=O)CCC1=O ; COc1cc2c(c(F)c1OCCCOc1c(OC)cc3sc(C(=O)CCC(=O)O[C@@H](C)CNC(=O)CCOCCOCCNC(=O)CN4C(=O)C=CC4=O)cc3c1F)CN(C(=O)CCC(=O)OC(C)(C)C)C2]

DIEA (22.2600 mg, 172.2345 mol) was added to a solution of N-succinimidyl maleimidoacetate (0.028 g, 111.0318 mol), J(S)-1-(3-(2-(2-aminoethoxy)ethoxy)propanamido)propan-2-yl 4-(5-(3-((2-(4-(tert-butoxy)-4-oxobutanoyl)-4-fluoro-6-methoxyisoindolin-5-yl)oxy)propoxy)-4-fluoro-6-methoxybenzo[b]thiophen-2-yl)-4-oxobutanoate (0.046 g, 51.4551 mol) in DMF (2 mL) at 25° C. The reaction mixture was stirred for 2 h at 25° C. The residue was purified on C18 column MeCN/water (0-65%). The purified fraction was concentrated and dried by lyohpilization at 25° C. There was [(1S)-2-[3-[2-[2-[[2-(2,5-dioxopyrrol-1-yl)acetyl]amino]ethoxy]ethoxy]propanoylamino]-1-methyl-ethyl]4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.053 g, 51.4025 mol, 99.8979% yield) obtained as a white solid. LCMS: (ESI, m/z): [M+H]⁺=1031.088.

### Step d:

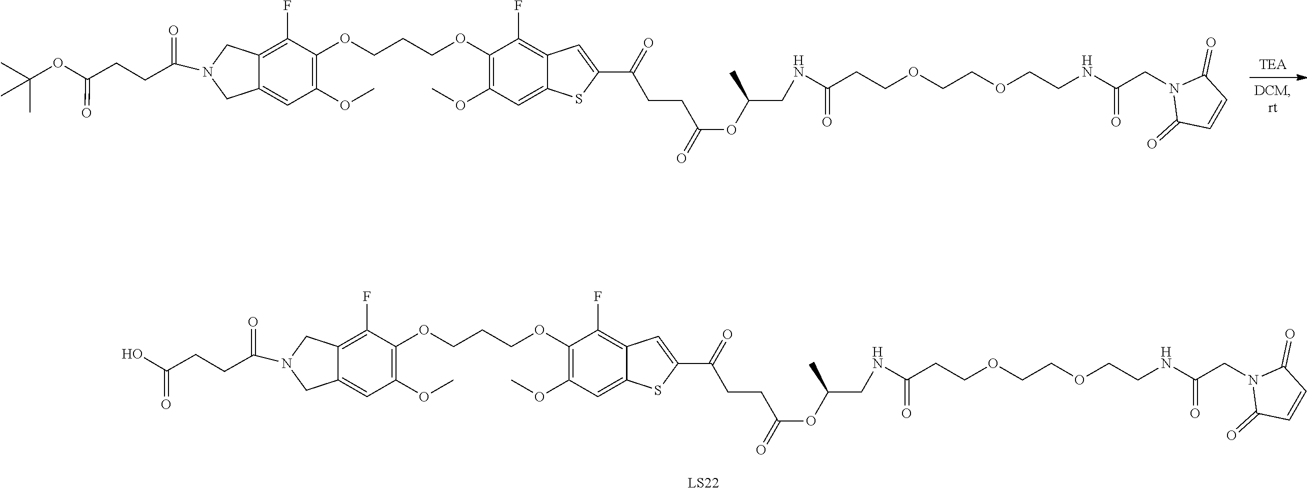 [SMILES: COc1cc2c(c(F)c1OCCCOc1c(OC)cc3sc(C(=O)CCC(=O)O[C@@H](C)CNC(=O)CCOCCOCCNC(=O)CN4C(=O)C=CC4=O)cc3c1F)CN(C(=O)CCC(=O)OC(C)(C)C)C2 ; COc1cc2c(c(F)c1OCCCOc1c(OC)cc3sc(C(=O)CCC(=O)O[C@@H](C)CNC(=O)CCOCCOCCNC(=O)CN4C(=O)C=CC4=O)cc3c1F)CN(C(=O)CCC(=O)O)C2]

Tert-butyl (S)-4-(5-(3-((2-(1-(2,5-dioxo-2,5-dihydro-1H-pyrrol-1-yl)-15-methyl-2,12,17-trioxo-6,9,16-trioxa-3,13-diazaicosan-20-oyl)-4-fluoro-6-methoxybenzo[b]thiophen-5-yl)oxy)propoxy)-4-fluoro-6-methoxyisoindolin-2-yl)-4-oxobutanoate (0.053 g, 51.4025 mol) was added to a solution of trifluoroacetic acid, TFA (1 mL) in DCM (5 mL) at 25° C. The reaction mixture was stirred for 2 h at 25° C. The reaction mixture was evaporated under reduced pressure. The residue was purified by Prep-HPLC. There was 4-[5-[3-[2-[4-[(1S)-2-[3-[2-[2-[[2-(2,5-dioxopyrrol-1-yl)acetyl]amino]ethoxy]ethoxy]propanoylamino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (27.4 mg, 28.1034 mol, 54.6731% yield) obtained as a white solid.

LCMS: (ESI, m/z): [M+H]⁺=974.980.

### Example IV-23 Synthesis of Linker-STING Agonist Compound LS23

### Step a:

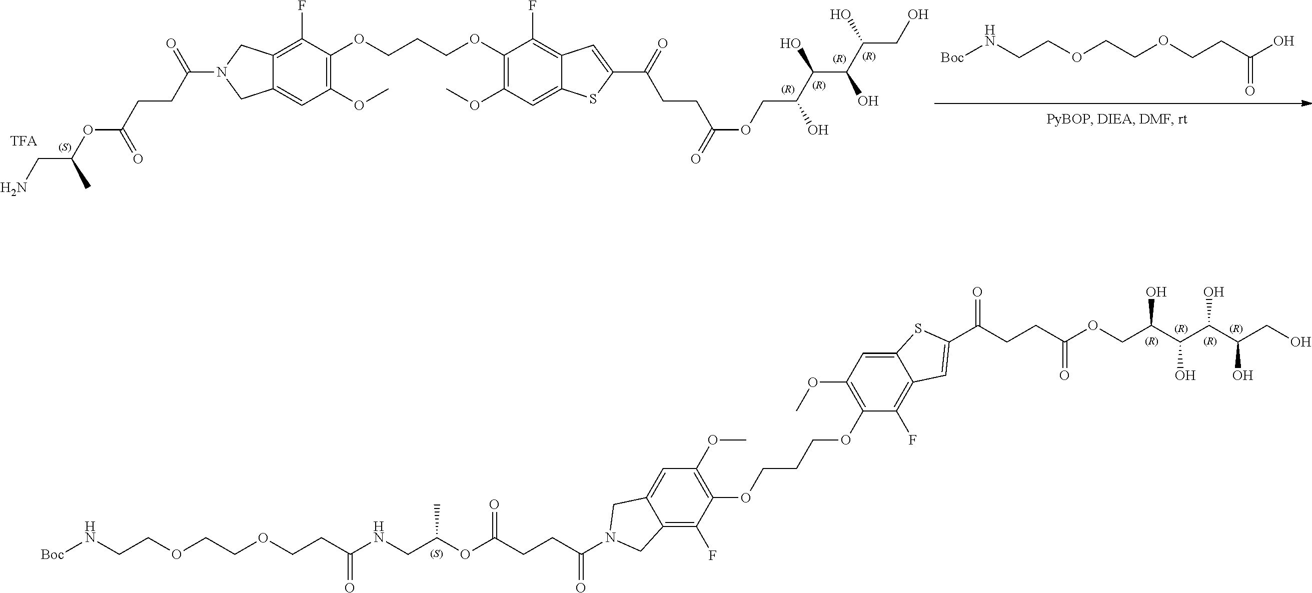

(S)-1-aminopropan-2-yl-4-(4-fluoro-5-(3-((4-fluoro-6-methoxy-2-(4-oxo-4- (((2R,3R,4R,5R)-2,3,4,5,6-pentahydroxyhexyl)oxy)butanoyl)benzo[b]thiophen-5-yl)oxy)propoxy)-6-methoxyisoindolin-2-yl)-4-oxobutanoate compound with 2,2,2-trifluoro-113-ethan-1-one (1:1) (0.0396 g, 46.9833 mol) and N,N-Diisopropylethylamine (0.0283 g, 218.9684 mol) were added to a solution of 2,2-dimethyl-4-oxo-3,8,11-trioxa-5-azatetradecan-14-oic acid (0.0222 g, 80.0539 mol) and PyBOP (0.0700 g, 134.5141 mol) in DMF (3 mL) at 20° C. under N₂ atmosphere. The reaction mixture was stirred for 2 h at 20° C. The reaction mixture was purified on C18 column ACN/H₂O (0.1% TFA)(0-35%). The purified fraction was concentrated and dried under vacuo. There was [(2R,3R,4R,5R)-2,3,4,5,6-pentahydroxyhexyl]4-[5-[3-[2-[4-[(1S)-2-[3-[2-[2-(tert- butoxycarbonylamino)ethoxy]ethoxy]propanoylamino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.11 g, 49.9025 mol, 50% purity, 53.1060% yield) obtained as a white oil, which was used directly to the next step.

LCMS: (ESI, m/z): [M+H]⁺=1102.420.

### Step b:

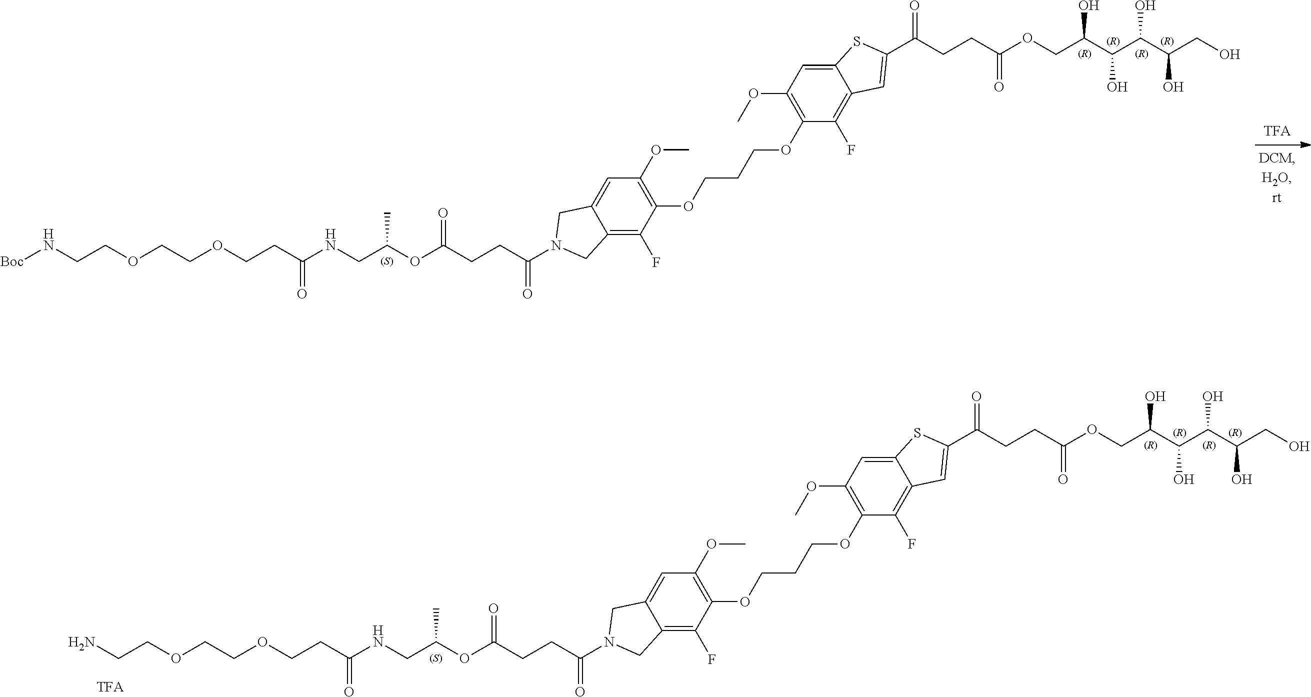

(S)-2,2-dimethyl-4,14-dioxo-3,8,11-trioxa-5,15-diazaoctadecan-17-yl 4-(4-fluoro-5-(3-((4-fluoro-6-methoxy-2-(4-oxo-4-(((2R,3R,4R,5R)-2,3,4,5,6-pentahydroxyhexyl)oxy)butanoyl)benzo[b]thiophen-5-yl)oxy)propoxy)-6-methoxyisoindolin-2-yl)-4-oxobutanoate (0.10 g, 50% purity, 45.3659 mol) was added to a solution of DCM (3 mL), Trifluoroacetic acid (3 mL) and water (0.3 mL) at 20° C. under open-air atmosphere. The reaction mixture was stirred for 1 h at 20° C. The reaction mixture was evaporated under reduced pressure. The residue was purified on C-18 column ACN/H₂O (0.1% FA)(0-25%). The purified fraction was concentrated and dried by lyophilization. There was (S)-1-(3-(2-(2-aminoethoxy)ethoxy)propanamido)propan-2-yl-4-(4-fluoro-5-(3-((4-fluoro-6-methoxy-2-(4-oxo-4-(((2R,3R,4R,5R)-2,3,4,5,6-pentahydroxyhexyl)oxy)butanoyl) benzo[b]thiophen-5-yl)oxy)propoxy)-6-methoxyisoindolin-2-yl)-4-oxobutanoate compound with 2,2,2-trifluoro-113-ethan-1-one (1:1) (0.026 g, 25.9472 mol, 57.1358% yield) obtained as a brown solid. LCMS: (ESI, m/z): [M+H]⁺=1002.360.

### Step c:

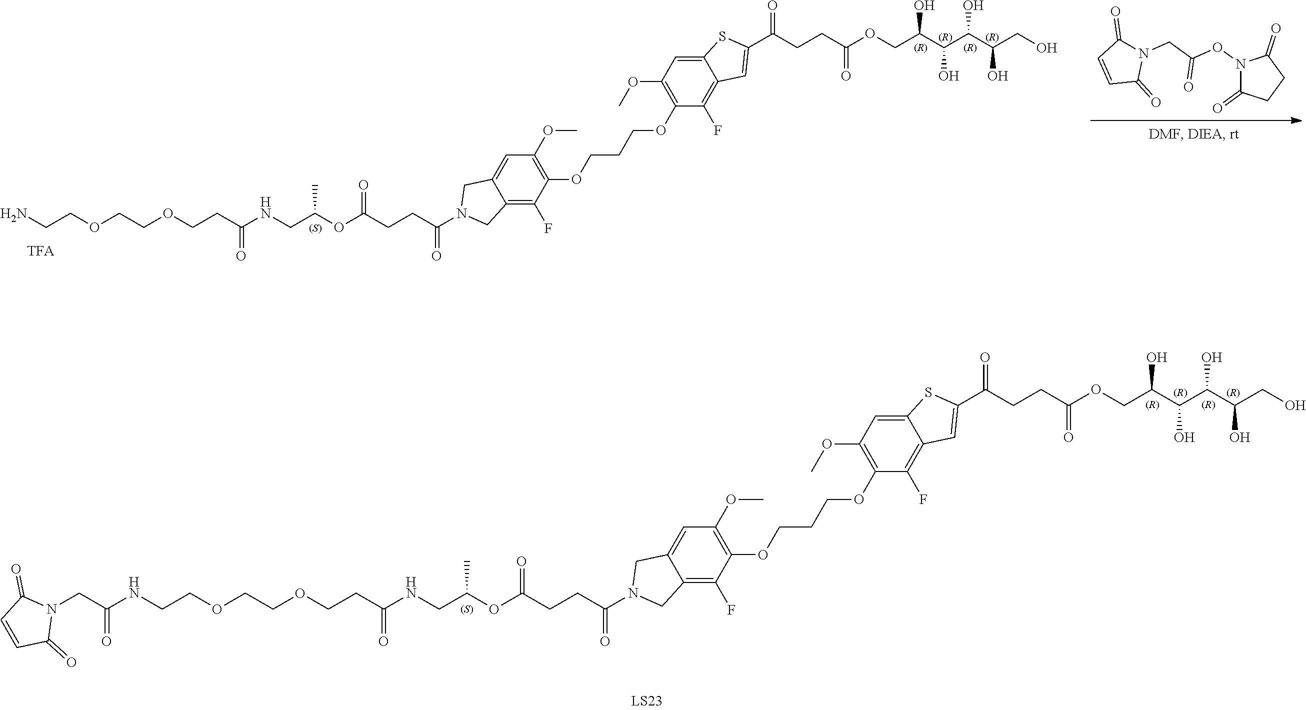

N,N-diisopropylethylamine (0.0125 g, 96.7175 mol) was added to a solution of N-succinimidyl maleimidoacetate (0.0139 g, 55.1194 mol) and (S)-1-(3-(2-(2-aminoethoxy)ethoxy)propanamido)propan-2-yl-4-(4-fluoro-5-(3-((4-fluoro-6-methoxy-2-(4-oxo-4-(((2R,3R,4R,5R)-2,3,4,5,6-pentahydroxyhexyl)oxy) butanoyl)benzo[b]thiophen-5-yl)oxy)propoxy)-6-methoxyisoindolin-2-yl)-4-oxobutanoate compound with 2,2,2-trifluoro-113-ethan-1-one (1:1) (0.0255 g, 25.4482 mol) in DMF (3 mL) at 20° C. under N₂ atmosphere.

The reaction mixture was stirred for 2 h at 20° C. The reaction mixture was purified on Prep-HPLC. The purified fraction was concentrated and dried by lyophilization. There was [(2R,3R,4R,5R)-2,3,4,5,6-pentahydroxyhexyl]-4-[5-[3-[2-[4-[(1S)-2-[3-[2-[2-[[2-(2,5-dioxopyrrol-1-yl)acetyl]amino]ethoxy]ethoxy]propanoylamino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.0147 g, 12.9046 mol, 50.7093% yield) obtained as a white solid. LCMS: (ESI, m/z): [M+H]⁺=1139.380.

### Example IV-24 Synthesis of Linker-STING Agonist Compound LS24

### Step a:

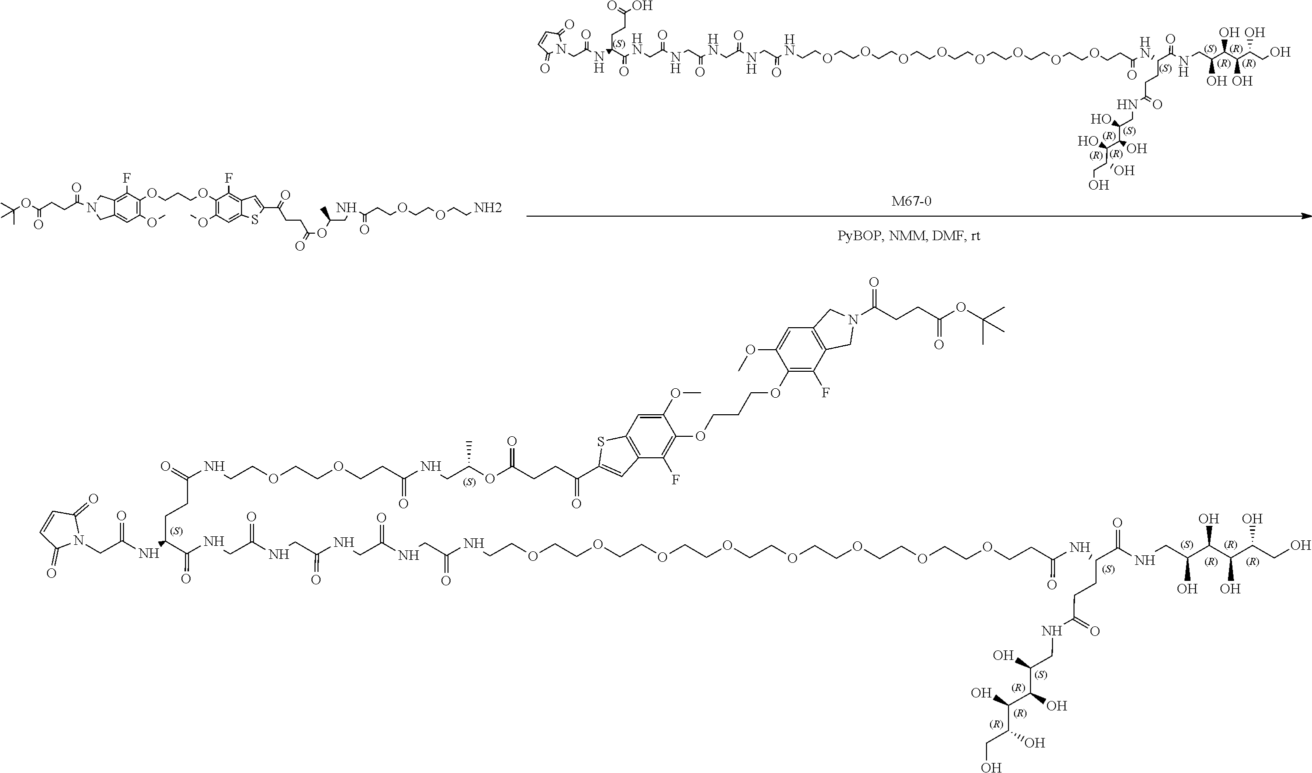

PyBOP (31.6 mg, 60.7236 mol) was added to a solution of (S)-1-(3-(2-(2-aminoethoxy)ethoxy)propanamido)propan-2-yl-4-(5-(3-((2-(4-(tert-butoxy)-4-oxobutanoyl)-4-fluoro-6-methoxyisoindolin-5-yl)oxy)propoxy)-4-fluoro-6-methoxybenzo[b]thiophen-2-yl)-4-oxobutanoate (47.9 mg, 53.5804 mol), (4S,47S,53S,54R,55R,56R)-4-(2-(2,5-dioxo-2,5-dihydro-1H-pyrrol-1-yl)acetamido)-53,54,55,56,57-pentahydroxy-5,8,11,14,17,45,50-heptaoxo-47-(((2S,3R,4R,5R)-2,3,4,5,6-pentahydroxyhexyl) carbamoyl)-21,24,27,30,33,36,39,42-octaoxa-6,9,12,15,18,46,51-heptaazaheptapentacontanoic acid (76.8 mg, 55.1970 mol) and NMM (15.6400 mg, 154.6265 mol) in DMF (1 mL) at 25° C. The reaction mixture was stirred for 2 h at 25° C. The residue was purified on C-18 column MeCN/Water (0-45%). The purified fraction was concentrated and dried under vacuo. There was [(1S)-2-[3-[2-[2-[[(4S)-4-[[2-(2,5-dioxopyrrol-1-yl)acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]pentanoyl]amino]ethoxy]ethoxy]propanoylamino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.082 g, 36.1656 mol, 67.4978% yield) obtained as a yollow oil.

LCMS: (ESI, m/z): [M+2H]⁺=1133.686.

### Step b:

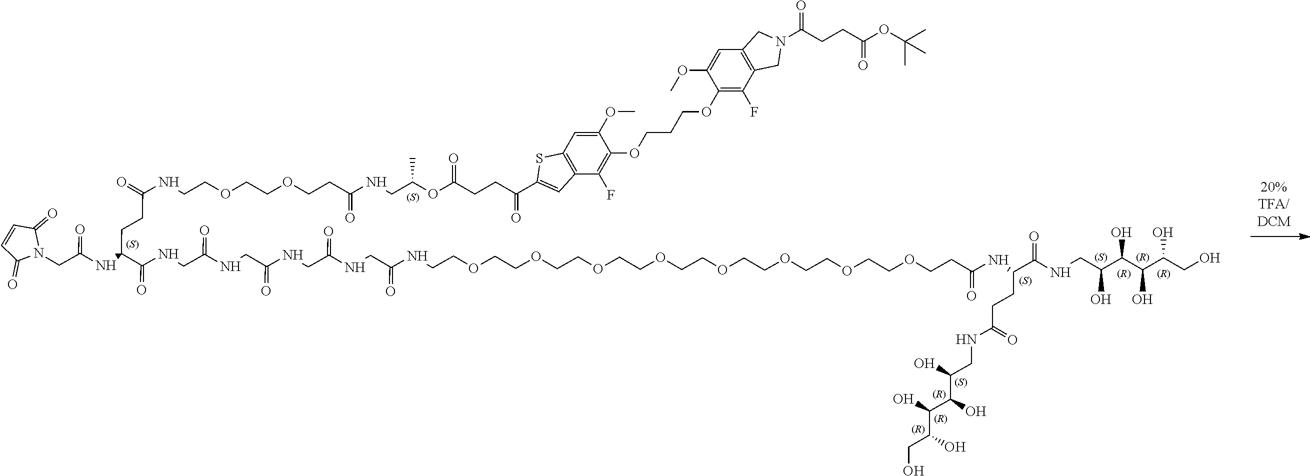

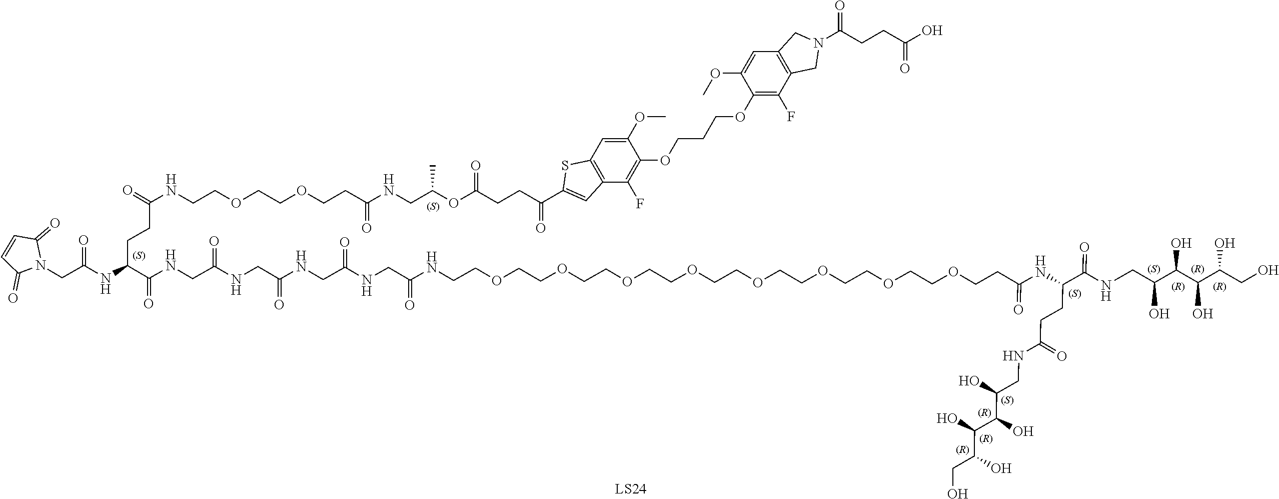

Tert-butyl-4-(5-(3-((2-((6S,22S,65S,71S,72R,73R,74R)-22-(2-(2,5-dioxo-2,5-dihydro-1H-pyrrol-1-yl)acetamido)-71,72,73,74,75-pentahydroxy-6-methyl-4,9,19,23,26,29,32,35,63,68-decaoxo-65-(((2S,3R,4R,5R)-2,3,4,5,6-pentahydroxyhexyl)carbamoyl)-5,12,15,39,42,45,48,51,54,57,60-undecaoxa-8,18,24,27,30,33,36,64,69-nonaazapentaheptacontanoyl)-4-fluoro-6-methoxybenzo[b]thiophen-5-yl)oxy) propoxy)-4-fluoro-6-methoxyisoindolin-2-yl)-4-oxobutanoate (0.082 g, 36.1656 mol) was added to a solution of TFA (1 mL) in DCM (5 mL) at 25° C. The reaction mixture was stirred for 2 h at 25° C. The reaction mixture was evaporated under reduced pressure. The residue was purified by Prep-HPLC. The purified fraction was concentrated and dried by lyohpilization at 25° C. There was 4-[5-[3-[2-[4-[(1S)-2-[3-[2-[2-[[(4S)-4-[[2-(2,5-dioxopyrrol-1-yl)acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]eth ylamino]ethyl]amino]ethyl]amino]ethyl]amino]ethyl]amino]pentanoyl]amino]ethoxy]ethoxy]propanoylamino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (23.3 mg, 10.5371 mol, 29.1356% yield) obtained as a white solid. LCMS: (ESI, m/z): [M+2H]²⁺=1105.633.

### Example IV-25 Synthesis of Linker-STING Agonist Compound LS25

### Step a:

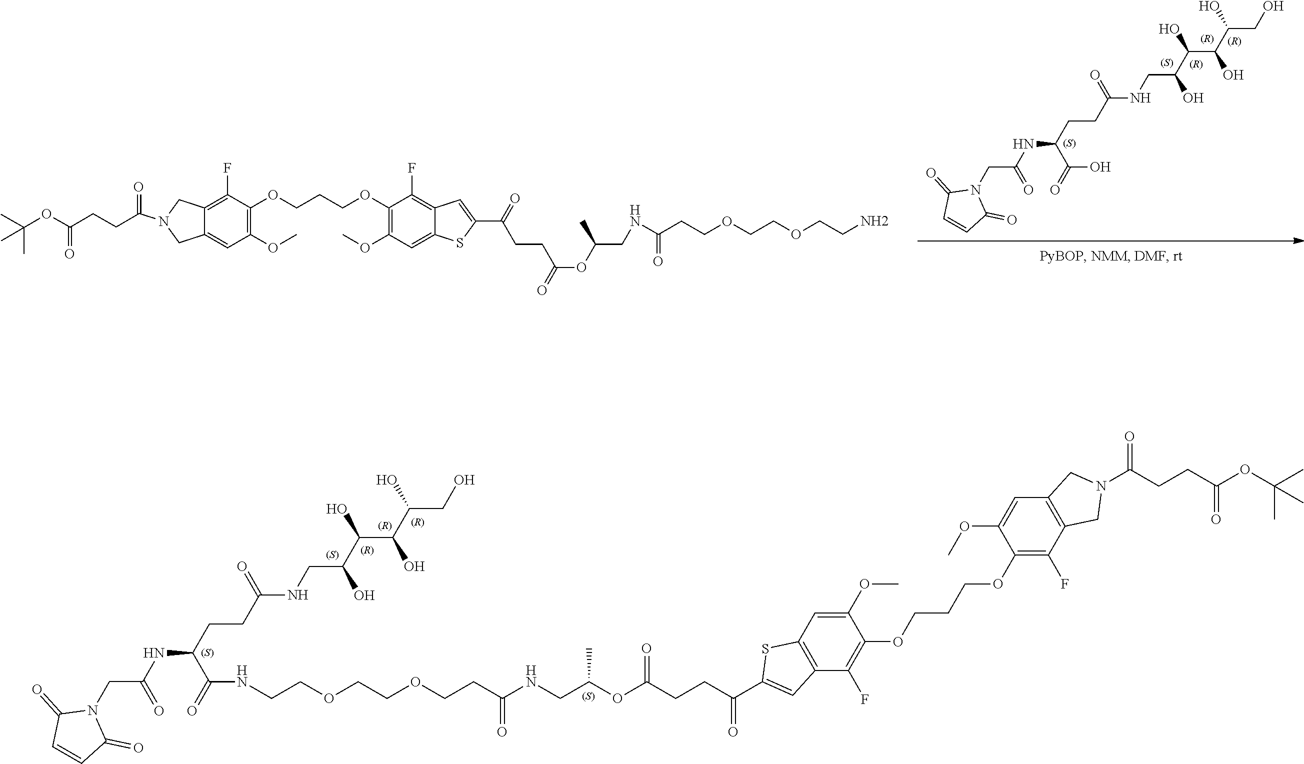

PyBOP (47.9 mg, 92.0462 mol) was added to a solution of N₂-(2-(2,5-dioxo-2,5-dihydro-1H-pyrrol-1-yl)acetyl)-N₅-((2S,3R,4R,5R)-2,3,4,5,6-pentahydroxyhexyl)-L-glutamine (40.8 mg, 91.1951 mol), (S)-1-(3-(2-(2-aminoethoxy)ethoxy)propanamido)propan-2-yl-4-(5-(3-((2-(4-(tert-butoxy)-4-oxobutanoyl)-4-fluoro-6-methoxyisoindolin-5-yl)oxy)propoxy)-4-fluoro-6-methoxybenzo[b]thiophen-2-yl)-4-oxobutanoate (41.5 mg, 46.4214 mol) and NMM (18.4000 mg, 181.9136 mol) in DMF (1 mL) at 25° C. The reaction mixture was stirred for 2 h at 25° C. The residue was purified on C18 column MeCN/Water (0-65%). The purified fraction was concentrated and dried by lyohpilization at 25° C. There was [(1S)-2-[3-[2-[2-[[(2S)-2-[[2-(2,5-dioxopyrrol-1-yl)acetyl]amino]-5-oxo-5-[[(2S,3R,4R,5R)-2,3,4,5,6-pentahydroxyhexyl]amino]pentanoyl]amino]ethoxy]ethoxy]propanoylamino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.049 g, 37.0269 mol, 79.7625% yield) obtained as a colorless oil. LCMS: (ESI, m/z): [M+H]⁺=1323.376.

### Step b:

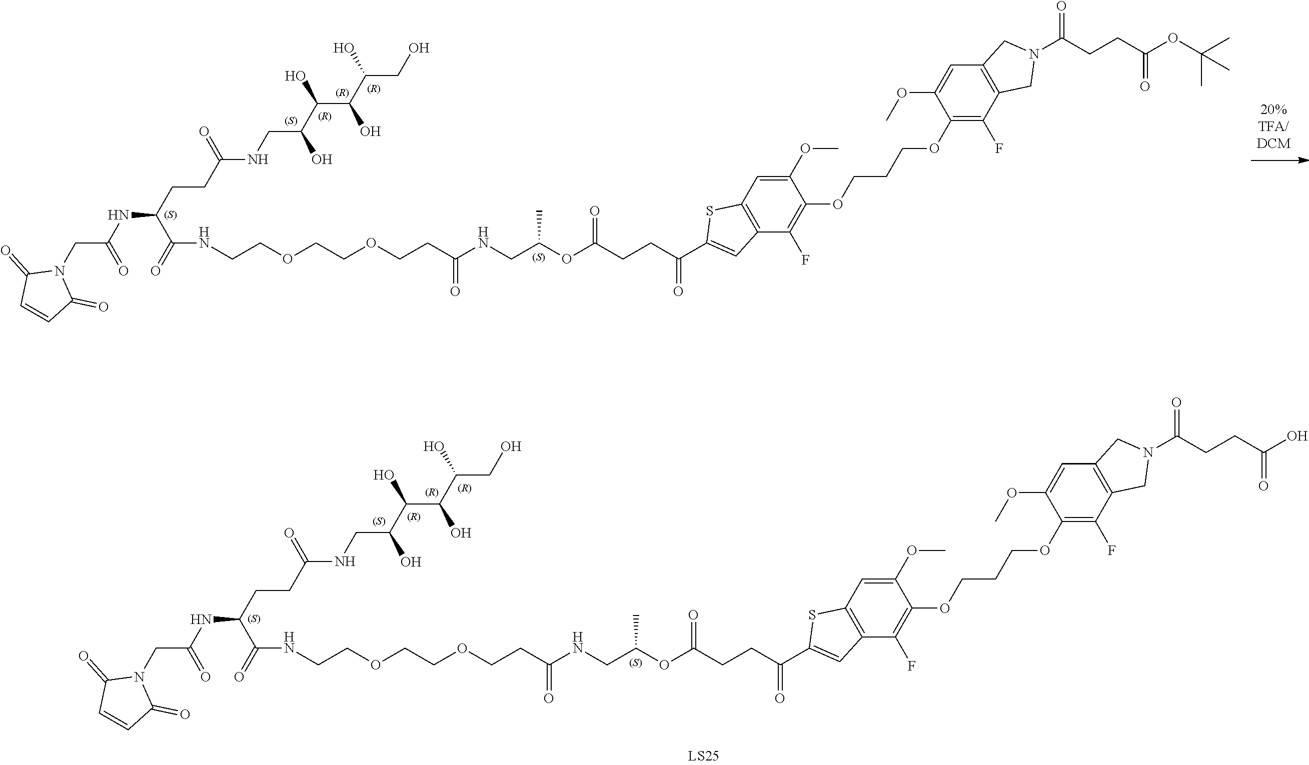

Tert-butyl-4-(5-(3-((2-((6S,20S,26S,27R,28R,29R)-20-(2-(2,5-dioxo-2,5-dihydro-1H-pyrrol-1-yl)acetamido)-26,27,28,29,30-pentahydroxy-6-methyl-4,9,19,23-tetraoxo-5,12,15-trioxa-8,18,24-triazatriacontanoyl)-4-fluoro-6-methoxybenzo[b]thiophen-5-yl)oxy) propoxy)-4-fluoro-6-methoxyisoindolin-2-yl)-4-oxobutanoate (0.044 g, 33.2486 mol) was added to a solution of TFA (1 mL) in DCM (5 mL) at 25° C. The reaction mixture was stirred for 2 h at 25° C. The reaction mixture was evaporated under reduced pressure. The residue was purified by Prep-HPLC. The purified fraction was concentrated and dried by lyohpilization at 25° C. There was 4-[5-[3-[2-[4-[(1S)-2-[3-[2-[2-[[(2S)-2-[[2-(2,5-dioxopyrrol-1-yl)acetyl]amino]-5-oxo-5-[[(2S,3R,4R,5R)-2,3,4,5,6-pentahydroxyhexyl]amino]pentanoyl]amino]ethoxy]ethoxy]propanoylamino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (6.0 mg, 4.7346 mol, 14.2401% yield) obtained as a white solid. LCMS: (ESI, m/z): [M+H]⁺=1267.268.

### Example IV-26 Synthesis of Linker-STING Agonist Compound LS26

### Step a:

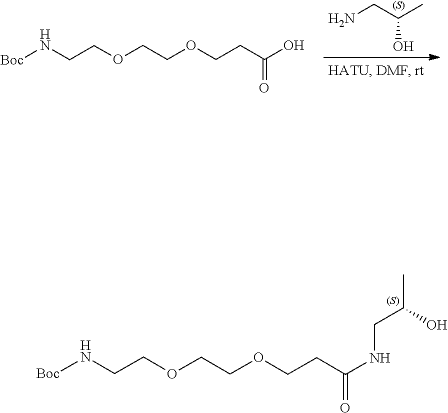

(S)-1-aminopropan-2-ol (2.81 g, 37.4121 mmol) was added to a solution of HATU (2.04 g, 5.3652 mmol) and 2,2-dimethyl-4-oxo-3,8,11-trioxa-5-azatetradecan-14-oic acid (1.00 g, 3.6060 mmol) in DMF (25 mL) at 20° C. under open-air atmosphere. The reaction mixture was stirred for 2 h at 20° C. The reaction mixture was purified on C-18 column ACN/H₂O (0.1% TFA)(0-30%). The purified fraction was concentrated and dried under vacuo. There was tert-butyl N-[2-[2-[3-[[(2S)-2-hydroxypropyl]amino]-3-oxo-propoxy]ethoxy]ethyl]carbamate (1.15 g, 3.4525 mmol, 95.7427% yield) obtained as a colorless oil. LCMS: (ESI, m/z): [M+H]⁺=335.210.

### Step b:

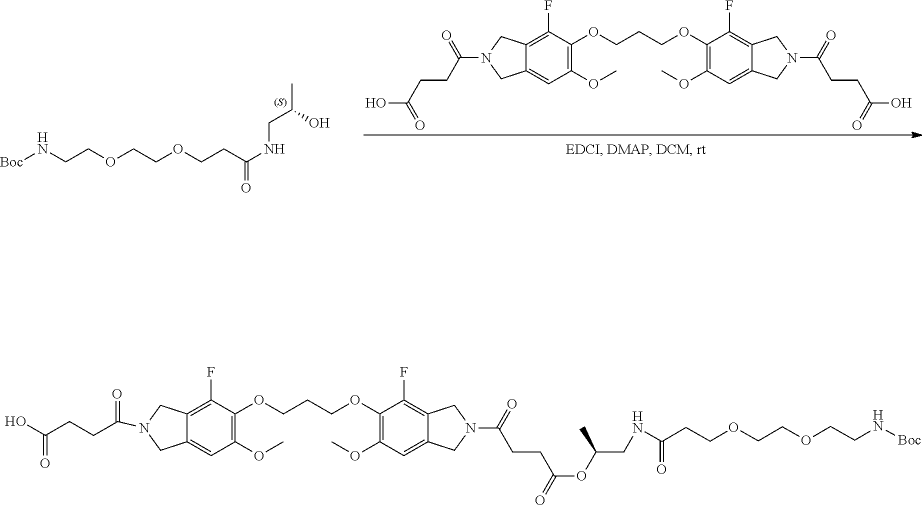

Tert-butyl N-[2-[2-[3-[[(2S)-2-hydroxypropyl]amino]-3-oxo-propoxy]ethoxy]ethyl]carbamate (0.066 g, 197.3643 mol) was added to a solution of 4,4′-((propane-1,3-diylbis(oxy))bis(4-fluoro-6-methoxyisoindoline-5,2-diyl))bis(4-oxobutanoic acid) (0.109 g, 179.6999 mol), EDCI (0.071 g, 370.3684 μmol) and N-(4-pyridyl)dimethylamine (0.037 g, 302.8641 mol) in DCM (10 mL) at 20° C. under open-air atmosphere. The reaction mixture was stirred for 2 h at 20° C. The reaction mixture was quenched with adding of water (100 mL) at 20° C., extracted with DCM (3×50 mL) and brine (50 mL). The organics were dried over Na₂SO₄, filtered and evaporated under reduced pressure. The residue was purified on C-18 column ACN/H₂O (0.1% TFA)(0-50%). The purified fraction was concentrated and dried under vacuo. There was 4-[5-[3-[2-[4-[(1S)-2-[3-[2-[2-(tert-butoxycarbonylamino)ethoxy]ethoxy]propanoylamino]-1-methyl-ethoxy]-4-oxo-butanoyl]-4-fluoro-6-methoxy-isoindolin-5-yl]oxypropoxy]-4-fluoro-6-methoxy-isoindolin-2-yl]-4-oxo-butanoic acid (0.09 g, 97.5124 mol, 54.2640% yield) obtained as a brown oil. LCMS: (ESI, m/z): [M+H]⁺=923.400.

### Step c:

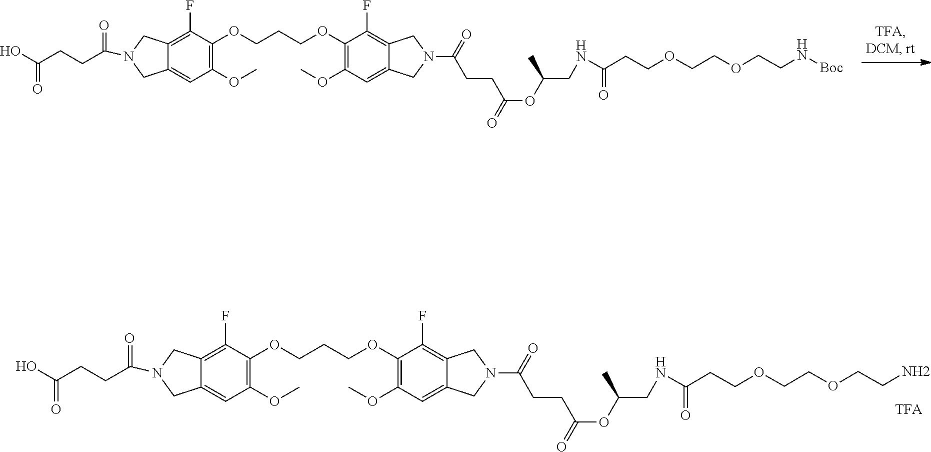 [SMILES: COc1cc2c(c(F)c1OCCCOc1c(OC)cc3c(c1F)CN(C(=O)CCC(=O)O[C@@H](C)CNC(=O)CCOCCOCCNC(=O)OC(C)(C)C)C3)CN(C(=O)CCC(=O)O)C2 ; COc1cc2c(c(F)c1OCCCOc1c(OC)cc3c(c1F)CN(C(=O)CCC(=O)O[C@@H](C)CNC(=O)CCOCCOCCN)C3)CN(C(=O)CCC(=O)O)C2]

4-[5-[3-[2-[4-[(1S)-2-[3-[2-[2-(tert-butoxycarbonylamino)ethoxy]ethoxy]propanoylamino]-1-methyl-ethoxy]-4-oxo-butanoyl]-4-fluoro-6-methoxy-isoindolin-5-yl]oxypropoxy]-4-fluoro-6-methoxy-isoindolin-2-yl]-4-oxo-butanoic acid (0.08 g, 86.6777 mol) was added to a solution of DCM (3 mL) and TFA (1 mL) at 20° C. under N₂ atmosphere. The reaction mixture was stirred for 1 h at 20° C. The reaction mixture was evaporated under reduced pressure. The residue was purified on C18 column ACN/H₂O (0.1% TFA)(0-20%). The purified fraction was concentrated and dried under vacuo. There was (S)-4-(5-(3-((2-(1-amino-12-methyl-9,14-dioxo-3,6,13-trioxa-10-azaheptadecan-17-oyl)-4-fluoro-6-methoxyisoindolin-5-yl)oxy)propoxy)-4-fluoro-6-methoxyisoindolin-2-yl)-4-oxobutanoic acid compound with 2,2,2-trifluoro-113-ethan-1-one (1:1) (0.063 g, 76.5637 mol, 88.3315% yield) obtained as a brown oil. LCMS: (ESI, m/z): [M+H]⁺=823.350.

### Step d:

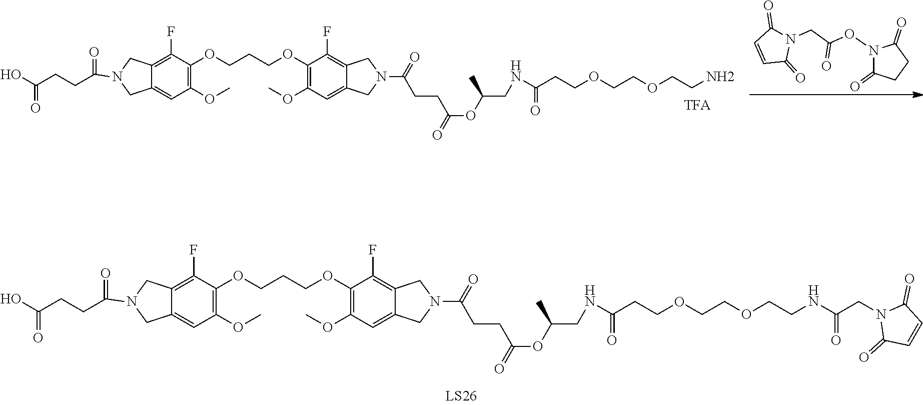 [SMILES: COc1cc2c(c(F)c1OCCCOc1c(OC)cc3c(c1F)CN(C(=O)CCC(=O)O[C@@H](C)CNC(=O)CCOCCOCCN)C3)CN(C(=O)CCC(=O)O)C2 ; O=C(CN1C(=O)C=CC1=O)ON1C(=O)CCC1=O ; COc1cc2c(c(F)c1OCCCOc1c(OC)cc3c(c1F)CN(C(=O)CCC(=O)O[C@@H](C)CNC(=O)CCOCCOCCNC(=O)CN1C(=O)C=CC1=O)C3)CN(C(=O)CCC(=O)O)C2]

N,N-diisopropylethylamine (0.034 g, 263.0716 mol) was added to a solution of N-succinimidyl maleimidoacetate (0.034 g, 134.8243 mol) and (S)-4-(5-(3-((2-(1-amino-12-methyl-9,14-dioxo-3,6,13-trioxa-10-azaheptadecan-17-oyl)-4-fluoro-6-methoxyisoindolin-5-yl)oxy)propoxy)-4-fluoro-6-methoxyisoindolin-2-yl)-4-oxobutanoic acid compound with 2,2,2-trifluoroacetaldehyde (1:1) (0.054 g, 65.6260 mol) in DMF (5 mL) at 20° C. under N₂ atmosphere. The reaction mixture was stirred for 2 h at 20° C. The reaction mixture was purified on C18 column ACN/H₂O (0.1% TFA)(0-45%). The purified fraction was concentrated and dried under vacuo. The residue was purified by Prep-HPLC. The purified fraction was concentrated and dried by lyophilization. There was 4-[5-[3-[2-[4-[(1S)-2-[3-[2-[2-[[2-(2,5-dioxopyrrol-1-yl)acetyl]amino]ethoxy]ethoxy]propanoylamino]-1-methyl-ethoxy]-4-oxo-butanoyl]-4-fluoro-6-methoxy-isoindolin-5-yl]oxypropoxy]-4-fluoro-6-methoxy-isoindolin-2-yl]-4-oxo-butanoic acid (0.0232 g, 24.1683 μmol, 36.8272% yield) obtained as a white solid. LCMS: (ESI, m/z): [M+H]⁺=960.360.

### Example IV-27 Synthesis of Linker-STING Agonist Compound LS27

### Step a:

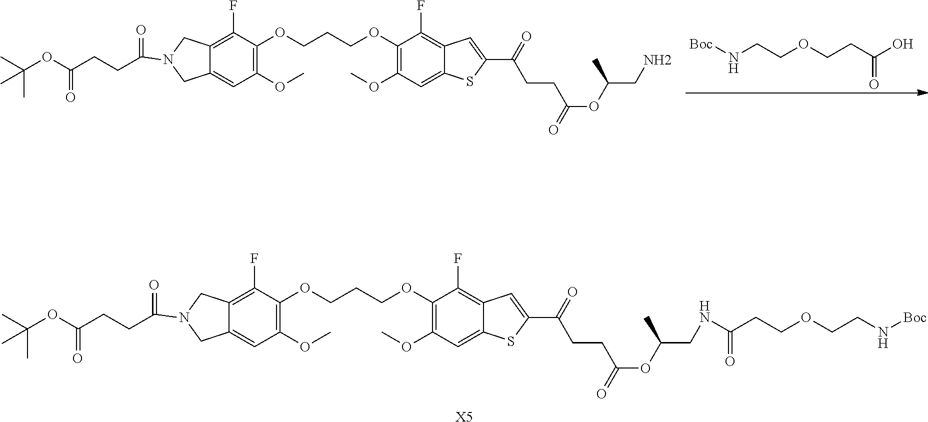 [SMILES: COc1cc2c(c(F)c1OCCCOc1c(OC)cc3sc(C(=O)CCC(=O)O[C@@H](C)CN)cc3c1F)CN(C(=O)CCC(=O)OC(C)(C)C)C2 ; CC(C)(C)OC(=O)NCCOCCC(=O)O ; COc1cc2c(c(F)c1OCCCOc1c(OC)cc3sc(C(=O)CCC(=O)O[C@@H](C)CNC(=O)CCOCCNC(=O)OC(C)(C)C)cc3c1F)CN(C(=O)CCC(=O)OC(C)(C)C)C2 ; "X5"]

(S)-1-aminopropan-2-yl-4-(5-(3-((2-(4-(tert-butoxy)-4-oxobutanoyl)-4-fluoro-6-methoxyisoindolin-5-yl)oxy)propoxy)-4-fluoro-6-methoxybenzo[b]thiophen-2-yl)-4-oxobutanoate (0.093 g, 126.5647 mol) was added to a solution of 3-(2-((tert-butoxycarbonyl)amino)ethoxy)propanoic acid (0.053 g, 227.2135 mol), N,N-diisopropylethylamine (0.0050 g, 38.6870 mol) and HATU(0.067 g, 176.2095 mol) in N,N-dimethylformamide (5 mL) at 25° C. under open-air atmosphere. The reaction mixture was stirred at 25° C. for 1h. The reaction mixture was evaporated under reduced pressure. The residue was purified on C-18 column ACN/H₂O(0-100%). The purified fraction was concentrated and dried under vacuo. There was [(1S)-2-[3-[2-(tert-butoxycarbonylamino)ethoxy]propanoylamino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.125 g, 131.5724 mol, 103.9566% yield) obtained as a white solid. LCMS: (ESI, m/z): [M+H]⁺=950.384.

### Step b:

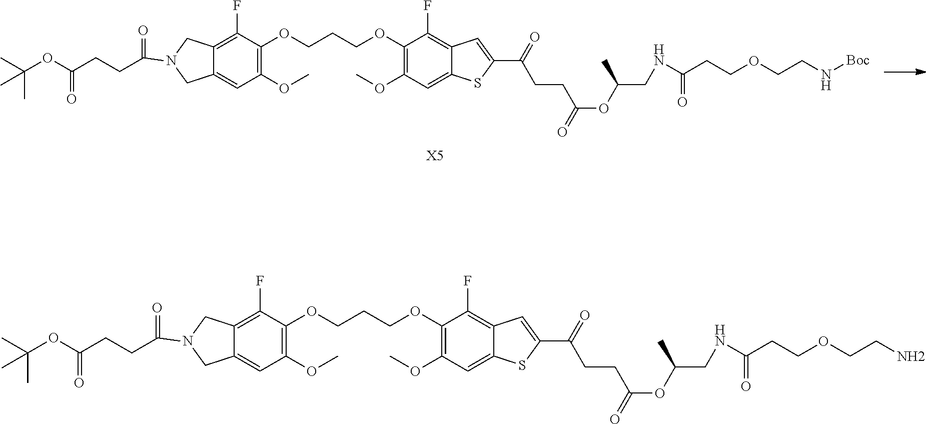 [SMILES: COc1cc2c(c(F)c1OCCCOc1c(OC)cc3sc(C(=O)CCC(=O)O[C@@H](C)CNC(=O)CCOCCNC(=O)OC(C)(C)C)cc3c1F)CN(C(=O)CCC(=O)OC(C)(C)C)C2 ; "X5" ; COc1cc2c(c(F)c1OCCCOc1c(OC)cc3sc(C(=O)CCC(=O)O[C@@H](C)CNC(=O)CCOCCN)cc3c1F)CN(C(=O)CCC(=O)OC(C)(C)C)C2]

Iodotrimethylsilane (0.040 g, 199.9070 mol) was added to a solution of [(1S)-2-[3-[2-(tert-butoxycarbonylamino)ethoxy]propanoylamino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.12 g, 126.3095 mol) in acetonitrile (10 mL) at 0° C. under open-air atmosphere. The reaction mixture was stirred for 0° C. lh. The reaction mixture was evaporated under reduced pressure. The residue was purified on C-18 column ACN/H20(0-100%). The purified fraction was concentrated and dried under vacuo. There was [(1S)-2-[3-(2-aminoethoxy)propanoylamino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (49

mg, 57.6517 mol, 45.6432% yield) obtained as a white solid. LCMS: (ESI, m/z): [M+H]⁺=850.332Step c:

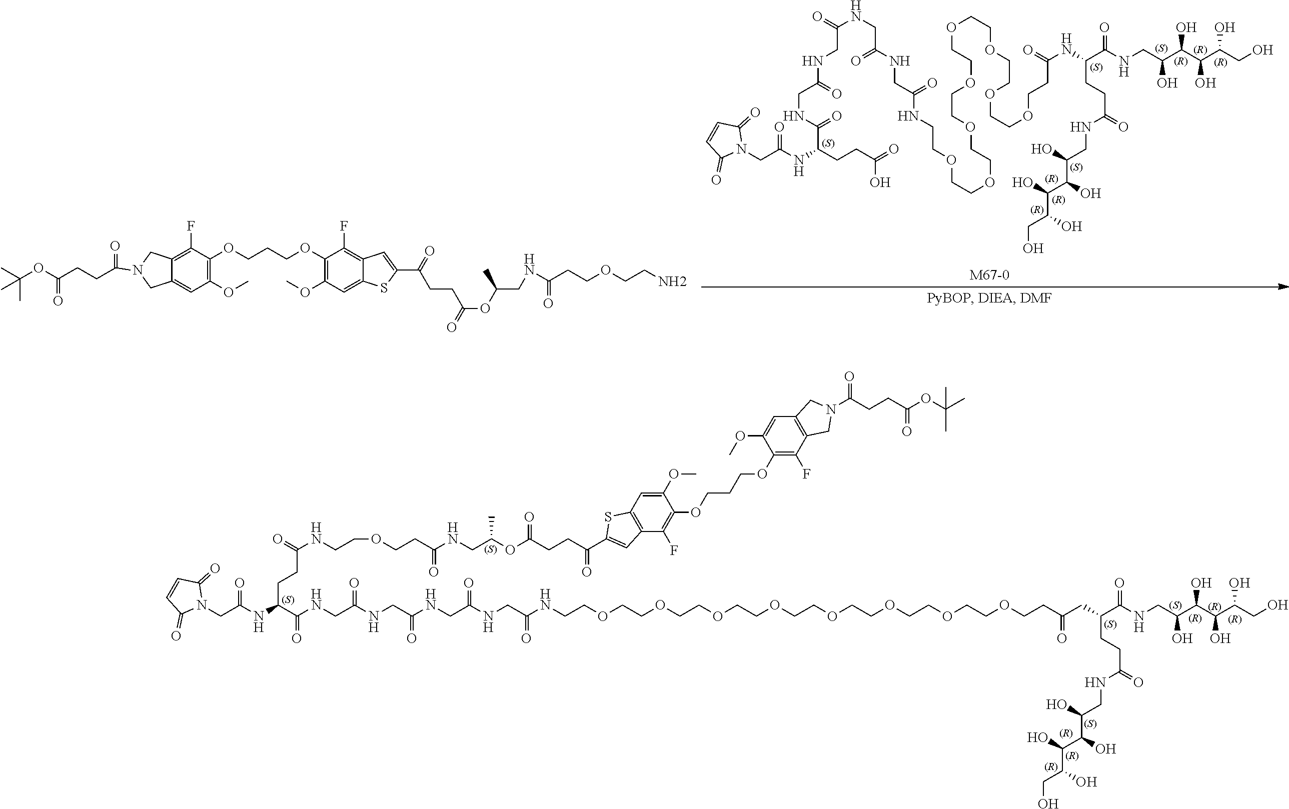

PyBOP (0.037 g, 71.1003 μmol) was added to a solution of [(1S)-2-[3-(2-aminoethoxy)propanoylamino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.050 g, 58.8282 mol), (4S,47S,53S,54R,55R,56R)-4-(2-(2,5-dioxo-2,5-dihydro-1H-pyrrol-1-yl) acetamido)-53,54,55,56,57-pentahydroxy-5,8,11,14,17,45,50-heptaoxo-47-(((2S,3R,4R,5R)-2,3,4,5,6-pentahydroxyhexyl)carbamoyl)-21,24,27,30,33,36,39,42-octaoxa-6,9,12,15,18,46,51-heptaazaheptapentacontanoic acid (0.085 g, 61.0905 μmol) and 4-methylmorpholine (0.030 g, 296.5980 mol) in N,N-dimethylformamide (5 mL) at 25° C. under open-air atmosphere. The reaction mixture was stirred at 25° C. for lh. The residue was purified on C18 column ACN/H₂O (0-100%). There was [(1S)-2-[3-[2-[[(4S)-4-[[2-(2,5-dioxopyrrol-1-yl)acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amin o]ethyl]amino]pentanoyl]amino]ethoxy]propanoylamino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (75 mg, 33.7337 mol, 57.3427% yield) obtained as a white solid. LCMS: (ESI, m/z): [M+2H]2+1112.467.

### Step d:

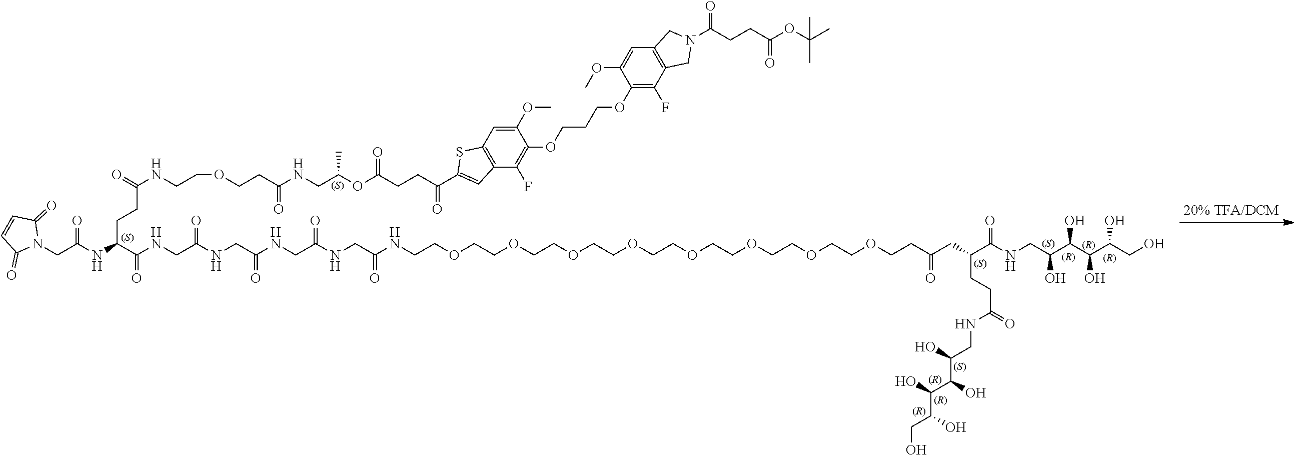

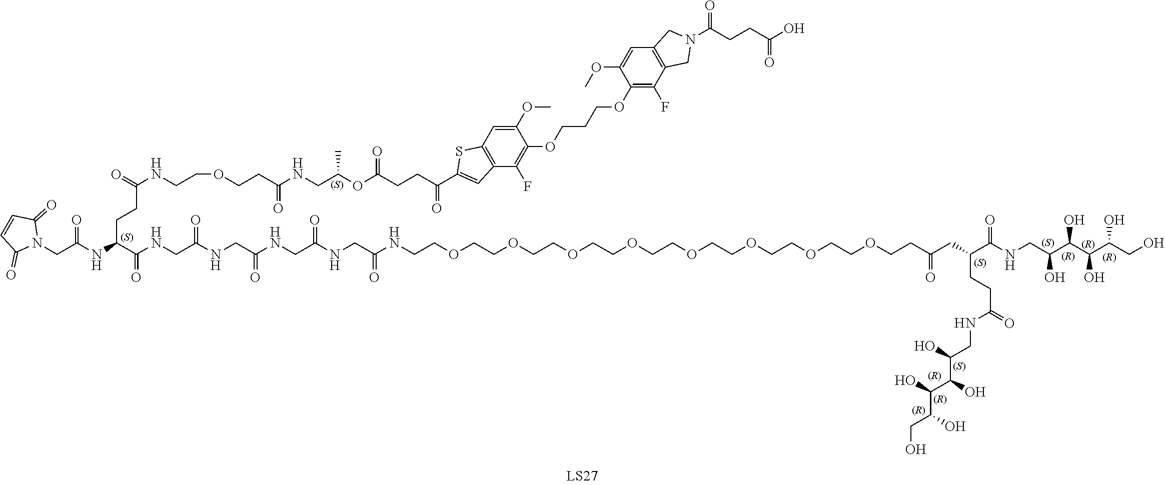

Trifluoroacetic acid, TFA (0.5 mL) was added to a solution of J[(1S)-2-[3-[2-[[(4S)-4-[[2-(2,5-dioxopyrrol-1-yl)acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]eth ylamino]ethyl]amino]ethyl]amino]ethyl]amino]ethyl]amino]pentanoyl]amino]ethoxy]propanoylamino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.075 g, 33.7336 mol) in DCM (2.5 mL) at 25° C. under open-air atmosphere. The reaction mixture was stirred for at 25° C. 1 h. The reaction mixture was evaporated under reduced pressure. The residue was purified by HPLC. There was 4-[5-[3-[2-[4-[(1S)-2-[3-[2-[[(4S)-4-[[2-(2,5-dioxopyrrol-1-yl)acetyl]amino]-5-oxo-5-[[2-oxo-2-[[2-oxo-2-[[2-oxo-2-[[2-oxo-2-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]ethyl]amino]ethyl]amino]ethyl]amino]ethyl]amino]pentanoyl]amino]ethoxy]propanoylamino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (17.4 mg, 8.0288 mol, 23.8007% yield) obtained as a white solid. LCMS: (ESI, m/z): [M+2H]⁺=1083.934.

### Example IV-28 Synthesis of Linker-STING Agonist Compound LS28

### Step a:

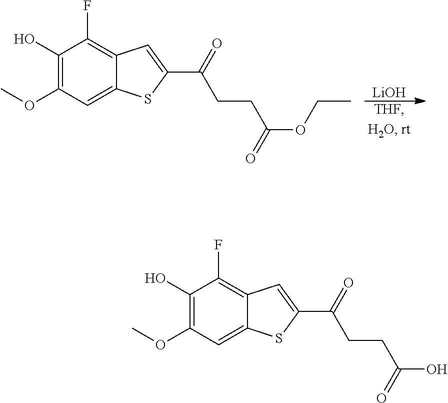

LiOH (0.74 g, 30.8999 mmol) was added to a mixture of ethyl-4-(4-fluoro-5-hydroxy-6-methoxybenzo[b]thiophen-2-yl)-4-oxobutanoate (2.01 g, 6.1592 mmol) in THF (15 mL) and Water (15 mL) at 20° C. The reaction mixture was stirred at 20° C. The reaction mixture was adjusted to pH=5 with HCl(1 μmol/L). The precipitate was collected by filtration, washed with H₂O (1×50 mL). The filter cake was dried by lyophilization. There was 4-(4-fluoro-5-hydroxy-6-methoxy-benzothiophen-2-yl)-4-oxo-butanoic acid (1.74 g, 5.8333 mmol, 94.7086% yield) obtained as a yellow solid. LCMS: (ESI, m/z): [M+H]⁺=299.030.

### Step b:

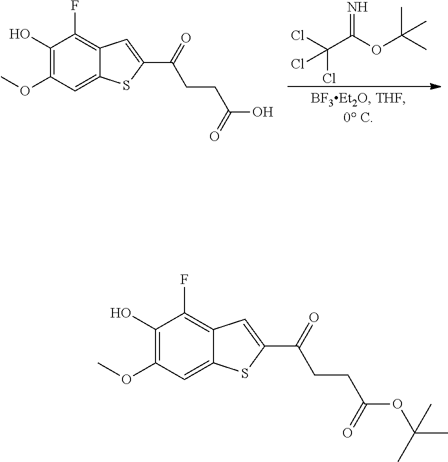 [SMILES: COc1cc2sc(C(=O)CCC(=O)O)cc2c(F)c1O ; CC(C)(C)OC(=N)C(Cl)(Cl)Cl ; COc1cc2sc(C(=O)CCC(=O)OC(C)(C)C)cc2c(F)c1O]

Boron trifluoride diethyl etherate (267.6426 mg, 905.1703 mol) was added to a solution of 4-(4-fluoro-5-hydroxy-6-methoxy-benzothiophen-2-yl)-4-oxo-butanoic acid (1.35 g, 4.5259 mmol) and tert-butyl 2,2,2-trichloroacetimidate (4.25 g, 19.4501 mmol) in THF (20 mL) at 0° C. under N₂ atmosphere. The reaction mixture was stirred 0.5 h at 0° C. The reaction mixture was concentrated and diluted with H₂O (100 mL), extracted with EA (3×200 mL) and brine (100 mL). The organics were dried over Na₂SO₄, filtered and evaporated under reduced pressure. The residue was purified on C18 column ACN/H₂O (0.1% TFA)(0-60%). The purified fraction was concentrated and dried under vacuo. There was tert-butyl 4-(4-fluoro-5-hydroxy-6-methoxy-benzothiophen-2-yl)-4-oxo-butanoate (1.26 g, 3.5554 mmol, 78.5572% yield) obtained as a yellow solid. LCMS: (ESI, m/z): [M+H]⁺=355.090. ¹H NMR (400 MHz, DMSO-d₆) δ 9.53 (s, 1H), 8.24 (s, 1H), 7.47 (s, 1H), 3.91 (s, 3H), 3.29 (t, J=6.4 Hz, 2H), 2.57 (t, J=6.4 Hz, 2H), 1.37 (d, J=1.6 Hz, 9H).

### Step c:

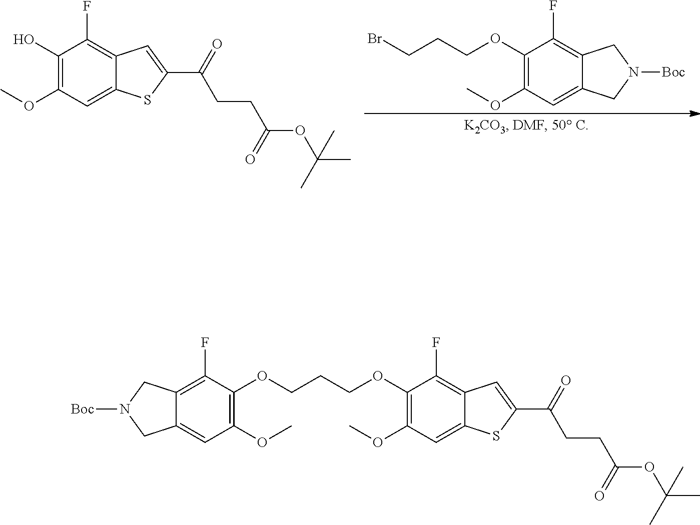 [SMILES: COc1cc2sc(C(=O)CCC(=O)OC(C)(C)C)cc2c(F)c1O ; COc1cc2c(c(F)c1OCCCBr)CN(C(=O)OC(C)(C)C)C2 ; COc1cc2c(c(F)c1OCCCOc1c(OC)cc3sc(C(=O)CCC(=O)OC(C)(C)C)cc3c1F)CN(C(=O)OC(C)(C)C)C2]

K₂CO₃ (0.99 g, 7.1632 mmol) was added to a solution of tert-butyl 4-(4-fluoro-5-hydroxy-6-methoxy-benzothiophen-2-yl)-4-oxo-butanoate (0.91 g, 2.5678 mmol) and tert-butyl 5-(3-bromopropoxy)-4-fluoro-6-methoxyisoindoline-2-carboxylate (1.00 g, 2.4736 mmol) in DMF (15 mL) at 25° C. under N₂ atmosphere. The reaction mixture was stirred at 50° C. The reaction mixture was purified on C18 column ACN/H₂O (0.1% TFA)(0-80%). The purified fraction was concentrated and dried under vacuo. There was tert-butyl-5-[3-[2-(4-tert-butoxy-4-oxo-butanoyl)-4-fluoro-6-methoxy- benzothiophen-5-yl]oxypropoxy]-4-fluoro-6-methoxy-isoindoline-2-carboxylate (1.30 g, 1.9181 mmol, 77.5435% yield) obtained as a yellow solid. LCMS: (ESI, m/z): [M+H]⁺=678.250. ¹H NMR (400 MHz, DMSO-d₆) δ 8.30 (s, 1H), 7.55 (s, 1H), 6.88 (s, 0.5H), 6.85 (s, 0.5H), 4.52 (s, 2H), 4.49 (s, 2H), 4.25 (t, J=6.1 Hz, 2H), 4.18 (t, J=6.1 Hz, 2H), 3.88 (d, J=1.9 Hz, 3H), 3.76 (d, J=4.8 Hz, 3H), 3.30 (t, J=6.3 Hz, 2H), 2.58 (t, J=6.3 Hz, 2H), 2.10-1.97 (m, 2H), 1.45 (s, 9H), 1.38 (s, 9H).

### Step d:

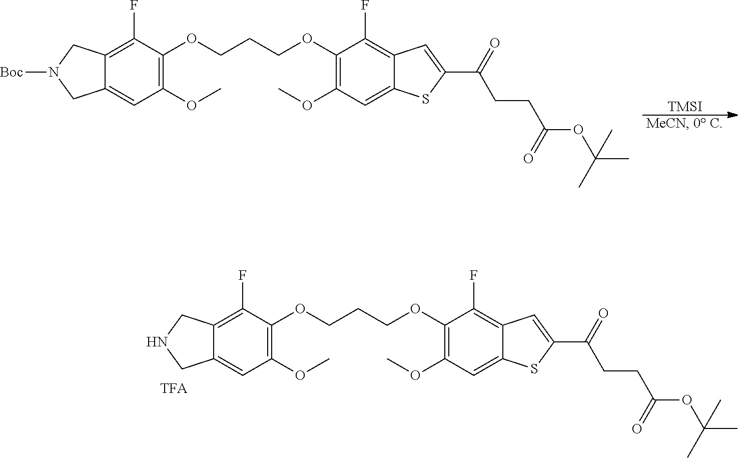 [SMILES: "TMSI" ; COc1cc2c(c(F)c1OCCCOc1c(OC)cc3sc(C(=O)CCC(=O)OC(C)(C)C)cc3c1F)CN(C(=O)OC(C)(C)C)C2 ; "MeCN, 0° C." ; COc1cc2c(c(F)c1OCCCOc1c(OC)cc3sc(C(=O)CCC(=O)OC(C)(C)C)cc3c1F)CNC2]

Iodotrimethylsilane (0.398 g, 1.9891 mmol) was added to a solution of tert-butyl 5-[3-[2-(4-tert-butoxy-4-oxo-butanoyl)-4-fluoro-6-methoxy-benzothiophen-5-yl]oxypropoxy]-4-fluoro-6-methoxy-isoindoline-2-carboxylate (1.145 g, 1.6894 mmol) in MeCN (20 mL) at 0° C. under N₂ atmosphere. The reaction mixture was stirred at 0° C. The reaction mixture was quenched with adding of H₂O (20 mL) at 0° C. The mixture was purified on C18 column ACN/H₂O (0.1% TFA)(0-60%). The purified fraction was concentrated and dried by lyophilization. There was tert-butyl-4-(4-fluoro-5-(3-((4-fluoro-6-methoxyisoindolin-5-yl)oxy)propoxy)-6-methoxybenzo[b]thiophen-2-yl)-4-oxobutanoate compound with 2,2,2-trifluoroacetaldehyde (1:1) (0.70 g, 1.2118 mmol, 71.7314% yield) obtained as a yellow solid. LCMS: (ESI, m/z): [M+H]⁺=578.190. ¹H NMR (400 MHz, DMSO-d₆) δ 9.62 (s, 2H), 8.31 (s, 1H), 7.57 (s, 1H), 6.97 (s, 1H), 4.53 (s, 2H), 4.48 (s, 2H), 4.29-4.18 (m, 4H), 3.88 (s, 3H), 3.78 (s, 3H), 3.30 (t, J=6.4 Hz, 2H), 2.58 (t, J=6.4 Hz, 2H), 2.10-2.00 (m, 2H), 1.38 (s, 9H).

### Step e:

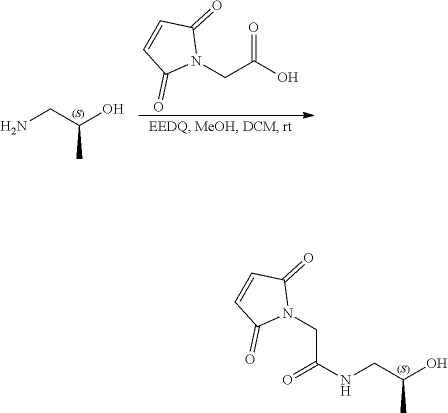

(S)-1-aminopropan-2-ol (0.270 g, 3.5948 mmol) was added to a solution of N-ethoxycarbonyl-2-ethoxy-1,2-dihydroquinoline (0.969 g, 3.9185 mmol) and 2-(2,5-dioxo-2,5-dihydro-1H-pyrrol-1-yl)acetic acid (0.504 g, 3.2493 mmol) in DCM (10 mL) at 20° C. under open-air atmosphere. The reaction mixture was stirred for 4 h at 20° C. The reaction mixture was evaporated under reduced pressure. The residue was purified on silica gel column MeOH/DCM(0-8%). The purified fraction was concentrated and dried under vacuo. There was 2-(2,5-dioxopyrrol-1-yl)-N-[(2S)-2-hydroxypropyl]acetamide (0.464 g, 2.1866 mmol, 67.2933% yield) obtained as a white solid. LCMS: (ESI, m/z): [M+H]⁺=213.080. ¹H NMR (400 MHz, DMSO-d₆) δ 8.11 (t, J=5.4 Hz, 1H), 7.09 (s, 2H), 4.69 (d, J=4.7 Hz, 1H), 4.03 (s, 2H), 3.66-3.59 (m, 1H), 3.04-2.94 (m, 2H), 1.00 (d, J=6.2 Hz, 3H).

### Step f:

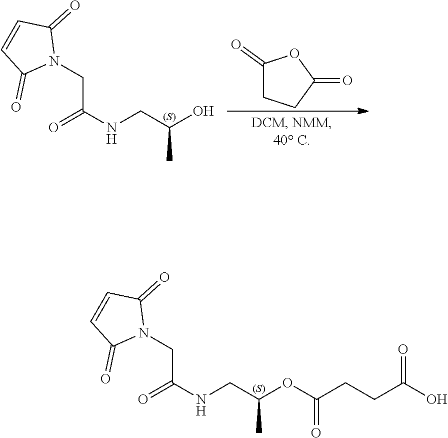

NMM (0.107 g, 1.0579 mmol) was added to a mixture of 2-(2,5-dioxopyrrol-1-yl)-N-[(2S)-2-hydroxypropyl]acetamide (0.099 g, 466.5366 mol) and dihydrofuran-2,5-dione (0.103 g, 1.0293 mmol) in DCM (5 mL) at 20° C. under open-air atmosphere. The reaction mixture was stirred overnight at 40° C. The mixture was purified on silica gel column MeOH/DCM(0-10%). The purified fraction was concentrated and dried under vacuo. There was 4-[(1S)-2-[[2-(2,5-dioxopyrrol-1-yl)acetyl]amino]-1-methyl-ethoxy]-4-oxo-butanoic acid (0.14 g, 448.3232 mol, 96.0960% yield ) obtained as a white solid. LCMS: (ESI, m/z): [M+H]⁺=313.100.

### Step g:

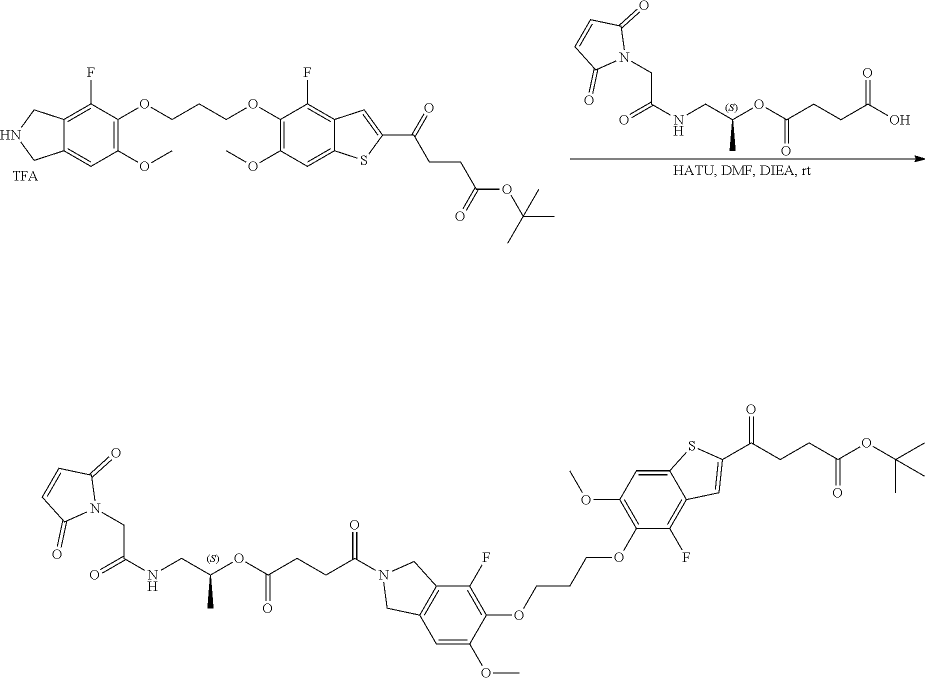

Tert-butyl-4-(4-fluoro-5-(3-((4-fluoro-6-methoxyisoindolin-5-yl)oxy)propoxy)-6-methoxybenzo[b]thiophen-2-yl)-4-oxobutanoate compound with 2,2,2-trifluoroacetaldehyde (1:1) (0.080 g, 138.4958 mol) was added to a solution of 4-[(1S)-2-[[2-(2,5-dioxopyrrol-1-yl)acetyl]amino]-1-methyl-ethoxy]-4-oxo-butanoic acid (0.056 g, 179.3291 mol) in DMF (3 mL) at 20° C. under open-air atmosphere. The reaction mixture was stirred for 2 h at 20° C. The reaction mixture was purified on C18 column ACN/H₂O (0.1% TFA)(0-80%). The purified fraction was concentrated and dried under vacuo. There was tert-butyl 4-[5-[3-[2-[4-[(1S)-2-[[2-(2,5-dioxopyrrol-1-yl)acetyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.15 g, 137.6313 mol, 76.7479% yield) obtained as a white solid. LCMS: (ESI, m/z): [M+H]⁺=872.280.

### Step h:

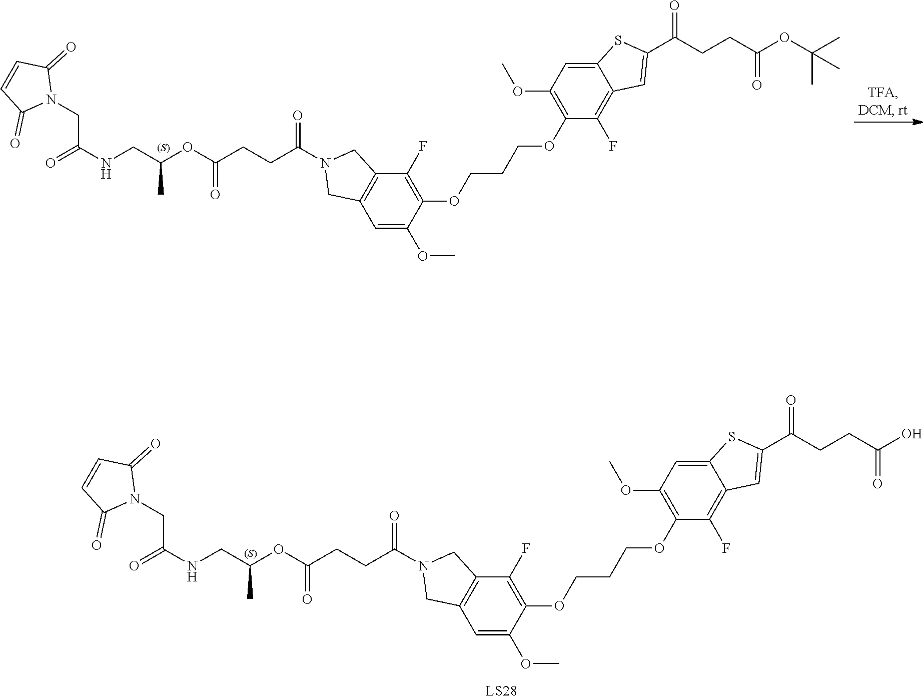

Tert-butyl-4-[5-[3-[2-[4-[(1S)-2-[[2-(2,5-dioxopyrrol-1-yl)acetyl]amino]-1-methyl-ethoxy]-4-oxo-butanoyl]-4-fluoro-6-methoxy-isoindolin-5-yl]oxypropoxy]-4-fluoro-6-methoxy-benzothiophen-2-yl]-4-oxo-butanoate (0.139 g, 159.4229 mol) was added to a solution of TFA (1 mL) and DCM (5 mL) at 20° C. under open-air atmosphere. The reaction mixture was stirred for 1 h at 20° C. The reaction mixture was concentrated under reduced pressure. The residue was purified by prep-HPLC. The purified fraction was lyophilized. There was 4-[5-[3-[2-[4-[(1S)-2-[[2-(2,5-dioxopyrrol-1-yl)acetyl]amino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (0.0502 g, 61.5355 mol, 44.7104% yield) obtained as a white solid. LCMS: (ESI, m/z): [M+H]⁺=816.220.

¹H NMR (400 MHz, DMSO-d₆) δ 8.30 (s, 1H), 8.26-8.19 (m, 1H), 7.56 (s, 1H), 7.09 (d, J=2.3 Hz, 2H), 6.90 (s, 0.5H), 6.87 (s, 0.5H), 4.88-4.81 (m, 1H), 4.80 (s, 2H), 4.58 (s, 1H), 4.55 (s, 1H), 4.25 (t, J=6.1, 1.8 Hz, 2H), 4.20 (t, J=6.1 Hz, 2H), 4.04 (s, 2H), 3.88 (s, 3H), 3.78 (d, J=2.4 Hz, 3H), 3.32 (t, J=6.4 Hz, 2H), 3.27-3.08 (m, 2H), 2.69-2.58 (m, 4H), 2.57-2.53 (m, 2H), 2.09-2.00 (m, 2H), 1.12 (d, J=6.3 Hz, 3H).

### Example IV-29 Synthesis of Linker-STING Agonist Compound LS29

### Step a:

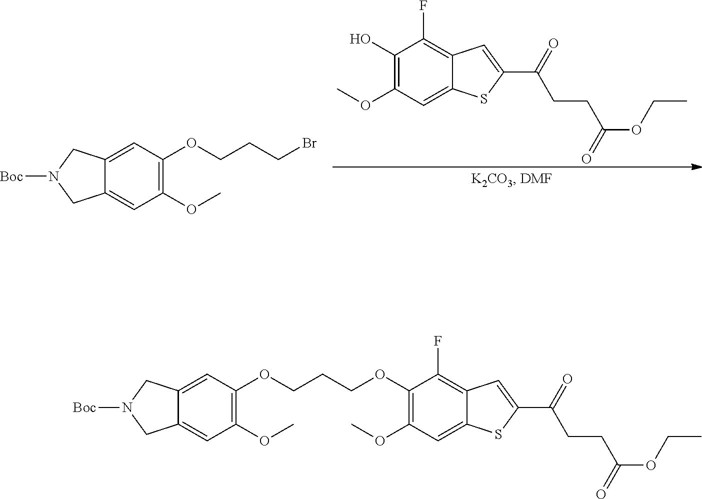 [SMILES: COc1cc2c(cc1OCCCBr)CN(C(=O)OC(C)(C)C)C2 ; CCOC(=O)CCC(=O)c1cc2c(F)c(O)c(OC)cc2s1 ; CCOC(=O)CCC(=O)c1cc2c(F)c(OCCCOc3cc4c(cc3OC)CN(C(=O)OC(C)(C)C)C4)c(OC)cc2s1]

K₂CO₃ (0.56 g, 4.0519 mmol) was added to a mixture of tert-butyl 5-(3-bromopropoxy)-6-methoxyisoindoline-2-carboxylate (0.53 g, 1.3721 mmol) and ethyl 4-(4-fluoro-5-hydroxy-6-methoxybenzo[b]thiophen-2-yl)-4-oxobutanoate (0.54 g, 1.6547 mmol) in DMF (15 mL) at 25° C. The reaction mixture was heated to 50° C. and stirred for 3 h. The resulting reaction mixture was filtered, and then was purified on C-18 column ACN/H₂O(0.1% FA)(0-80%). The purified fraction was concentrated and lyophilized overnight. There was tert-butyl 5-[3-[2-(4-ethoxy-4-oxo-butanoyl)-4-fluoro-6- methoxy-benzothiophen-5-yl]oxypropoxy]-6-methoxy-isoindoline-2-carboxylate (0.83 g, 95.7610% yield) obtained as a yellowish solid. LCMS: (ESI, m/z): [M+H-Boc]⁺=532.200.

### Step b:

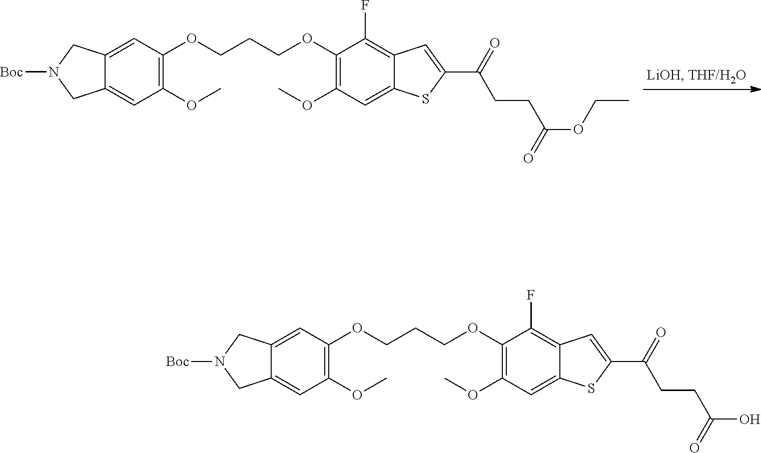 [SMILES: CCOC(=O)CCC(=O)c1cc2c(F)c(OCCCOc3cc4c(cc3OC)CN(C(=O)OC(C)(C)C)C4)c(OC)cc2s1 ; COc1cc2c(cc1OCCCOc1c(OC)cc3sc(C(=O)CCC(=O)O)cc3c1F)CN(C(=O)OC(C)(C)C)C2]

LiOH (0.080 g, 3.3405 mmol) was added to a solution of tert-butyl 5-[3-[2-(4-ethoxy-4-oxo-butanoyl)-4-fluoro-6-methoxy-benzothiophen-5-yl]oxypropoxy]-6-methoxy-isoindoline-2-carboxylate (0.825 g, 1.3060 mmol) in THF (15 mL) and H20 (5 mL) at 25° C. The reaction mixture was stirred for 2 h at 25° C. The resulting reaction mixture was adjusted with 2 N HCl solution to PH=5, and then extracted with EA. The organic layer was concentrated under reduced pressure. The residue was lyophilized overnight. There was 4-[5-[3-(2-tert-butoxycarbonyl-6-methoxy-isoindolin-5-yl) oxypropoxy]-4-fluoro-6-methoxy-benzothiophen-2-yl]-4-oxo-butanoic acid (0.76 g, 96.4022% yield) obtained as a yellowish solid. LCMS: (ESI, m/z): [M+H-Boc]⁺=504.200.

### Step c:

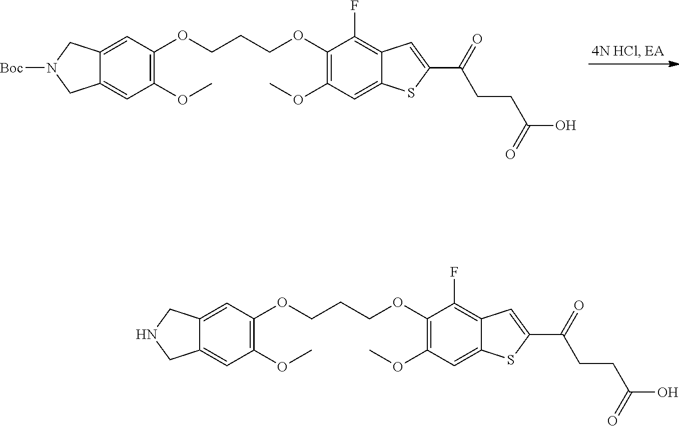 [SMILES: COc1cc2c(cc1OCCCOc1c(OC)cc3sc(C(=O)CCC(=O)O)cc3c1F)CN(C(=O)OC(C)(C)C)C2 ; COc1cc2c(cc1OCCCOc1c(OC)cc3sc(C(=O)CCC(=O)O)cc3c1F)CNC2]

4-[5-[3-(2-tert-butoxycarbonyl-6-methoxy-isoindolin-5-yl) oxypropoxy]-4-fluoro-6-methoxy-benzothiophen-2-yl]-4-oxo-butanoic acid (0.166 g, 274.9918 mol) was dissolved in 4 N HCl solution (8 mL) and EA (8 mL) at 25° C. The reaction mixture was stirred for 1 h at 25° C. The resulting reaction mixture was concentrated under reduced pressure. There was 4-[4-fluoro-6-methoxy-5-[3-(6-methoxyisoindolin-5-yl) oxypropoxy]benzothiophen-2-yl]-4-oxo-butanoic acid (0.13 g, 93.8838% yield, HCl salt) obtained as a brown solid. LCMS: (ESI, m/z): [M+H]⁺=504.250.

### Step d:

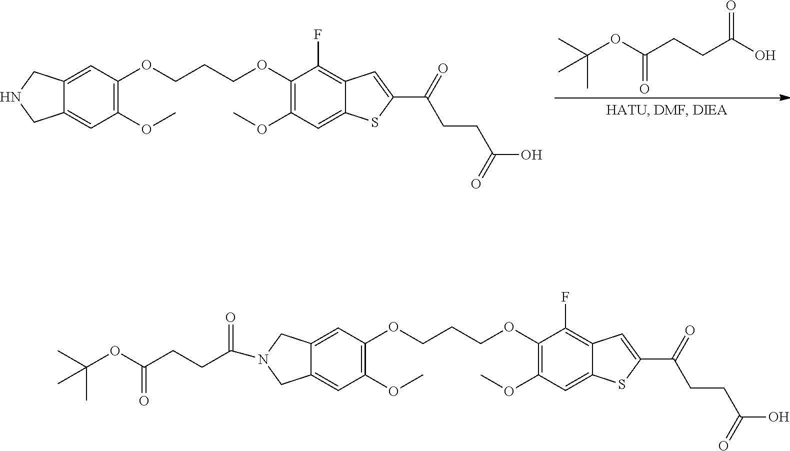 [SMILES: COc1cc2c(cc1OCCCOc1c(OC)cc3sc(C(=O)CCC(=O)O)cc3c1F)CNC2 ; CC(C)(C)OC(=O)CCC(=O)O ; COc1cc2c(cc1OCCCOc1c(OC)cc3sc(C(=O)CCC(=O)O)cc3c1F)CN(C(=O)CCC(=O)OC(C)(C)C)C2]

HATU (0.174 g, 457.6186 mol) was added to a solution of 4-(tert-butoxy)-4-oxobutanoic acid (0.547 g, 3.1585 mmol) in DMF (3 mL) at 25° C. The reaction mixture was stirred for 0.5 h at 25° C. A mixture of 4-[4-fluoro-6-methoxy-5-[3-(6-methoxyisoindolin-5-yl)oxypropoxy]benzothiophen-2-yl]-4-oxo-butanoic acid (0.013 g, 25.8173 mol) and DIEA (0.010 mL, 57.4115 mol) in DMF (2 mL) was added dropwise. The reaction mixture was continually stirred for 2 h. The resulting aqueous solution was purified on C18 column ACN/H₂O(0.1% FA)(0-70%). The purified fraction was concentrated and lyophilized overnight. There was 4-[5-[3-[2-(4-tert-butoxy-4-oxo-butanoyl)-6-methoxy-isoindolin-5-yl]oxypropoxy]-4-fluoro-6-methoxy-benzothiophen-2-yl]-4-oxo-butanoic acid (0.16 g, 93.9402% yield) obtained as an off-white solid. LCMS: (ESI, m/z): [M+H]⁺=660.300.

### Step e:

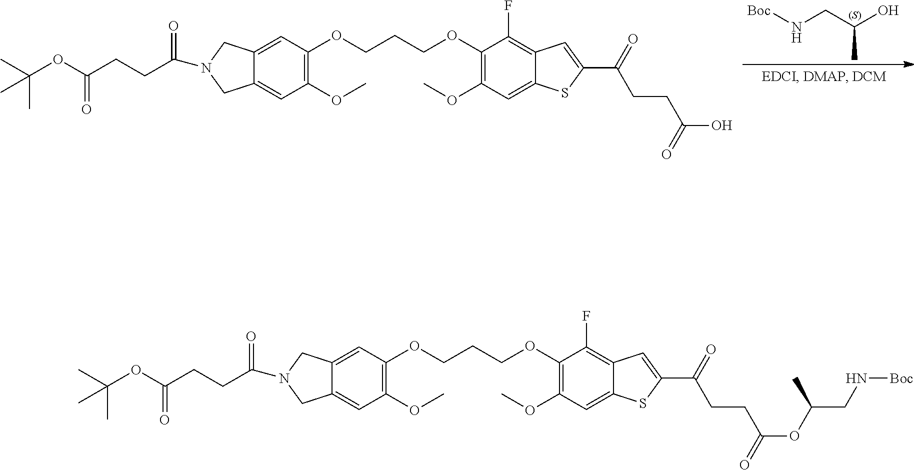

EDCI (0.118 g, 615.5419 mol) was added to a solution of 4-[5-[3-[2-(4-tert-butoxy-4-oxo-butanoyl)-6-methoxy-isoindolin-5-yl]oxypropoxy]-4-fluoro-6-methoxy-benzothiophen-2-yl]-4-oxo-butanoic acid (0.160 g, 242.5281 mol), tert-butyl (S)-(2-hydroxypropyl)carbamate (0.136 g, 776.1458 mol), and DMAP (0.025 g, 204.6379 mol) in DCM (15 mL) at 25° C. The reaction mixture was heated to 35° C. and stirred for 2 h. The reaction mixture was concentrated under reduced pressure. The resulting residue was purified on C-18 column ACN/0.1% TFA-H₂O(0-80%). The purified fraction was concentrated under reduced pressure. There was [(1S)-2-(tert-butoxycarbonylamino)-1- methyl-ethyl]-4-[5-[3-[2-(4-tert-butoxy-4-oxo-butanoyl)-6-methoxy-isoindolin-5-yl]oxypropoxy]-4-fluoro-6-methoxy-benzothiophen-2-yl]-4-oxo-butanoate (0.19 g, 95.8977% yield) obtained as a brown solid. LCMS: (ESI, m/z): [M+H]⁺=817.350.

### Step f:

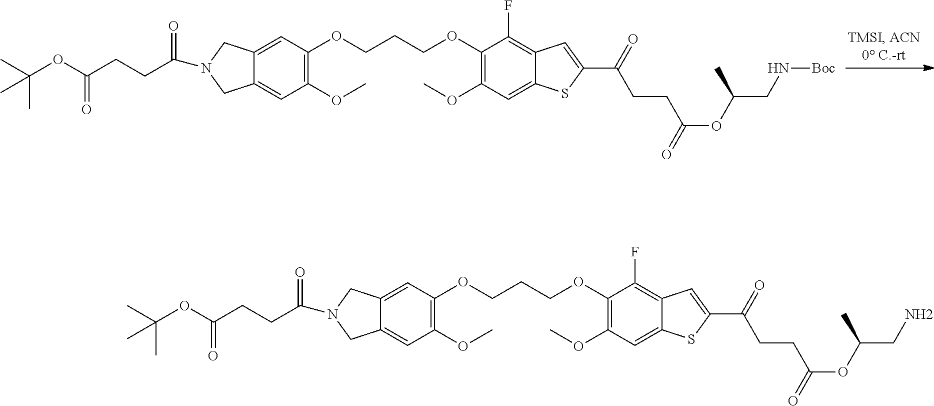 [SMILES: COc1cc2c(cc1OCCCOc1c(OC)cc3sc(C(=O)CCC(=O)O[C@@H](C)CNC(=O)OC(C)(C)C)cc3c1F)CN(C(=O)CCC(=O)OC(C)(C)C)C2 ; COc1cc2c(cc1OCCCOc1c(OC)cc3sc(C(=O)CCC(=O)O[C@@H](C)CN)cc3c1F)CN(C(=O)CCC(=O)OC(C)(C)C)C2]

A solution of iodotrimethylsilane solution (1 mL, 70 mg/mL, 348.8684 mol) in CAN was added to a solution of [(1S)-2-(tert-butoxycarbonylamino)-1-methyl-ethyl]-4-[5-[3-[2-(4-tert- butoxy-4-oxo-butanoyl)-6-methoxy-isoindolin-5-yl]oxypropoxy]-4-fluoro-6-methoxy-benzothiophen-2-yl]-4-oxo-butanoate (0.19 g, 232.5789 mol) in ACN (6 mL) at 25° C. The reaction mixture was stirred for 0.5 h at 0° C., and then warmed to room temperature and continually stirred for 1.5 h. The reaction mixture was quenched with H₂O (8 mL).

The resulting reaction mixture was purified on C18 column ACN/0.1% TFA-H20(0-70%). The purified fraction was concentrated under reduced pressure. There was [(1S)-2-amino-1-methyl-ethyl]-4-[5-[3-[2-(4-tert-butoxy-4-oxo-butanoyl)-6-methoxy-isoindolin-5-yl]oxypropoxy]-4-fluoro-6-methoxy-benzothiophen-2-yl]-4-oxo-butanoate (0.12 g, 71.9790% yield) obtained as a brown semi-solid. LCMS: (ESI, m/z): [M+H]⁺=717.350.

### Step g:

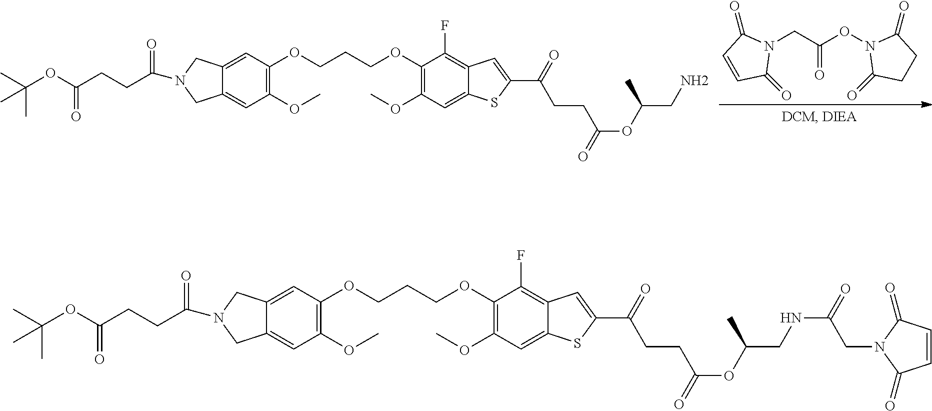 [SMILES: COc1cc2c(cc1OCCCOc1c(OC)cc3sc(C(=O)CCC(=O)O[C@@H](C)CN)cc3c1F)CN(C(=O)CCC(=O)OC(C)(C)C)C2 ; O=C(CN1C(=O)C=CC1=O)ON1C(=O)CCC1=O ; COc1cc2c(cc1OCCCOc1c(OC)cc3sc(C(=O)CCC(=O)O[C@@H](C)CNC(=O)CN4C(=O)C=CC4=O)cc3c1F)CN(C(=O)CCC(=O)OC(C)(C)C)C2]

N-succinimidyl maleimidoacetate (0.077 g, 305.3375 mol) was added to a solution of [(1S)-2-amino-1-methyl-ethyl]-4-[5-[3-[2-(4-tert-butoxy-4-oxo-butanoyl)-6-methoxy-isoindolin-5-yl]oxypropoxy]-4-fluoro-6-methoxy-benzothiophen-2-yl]-4-oxo-butanoate (0.120 g, 167.4079 mol) and DIEA (33.3900 mg, 258.3517 μmol) in DCM (5 mL) at 25° C. The reaction mixture was stirred for 2 h at 25° C. The reaction mixture was concentrated under reduced pressure. The crude was further purified by prep-HPLC (the mobile phase: ACN/0.1% TFA-H₂O(0-60%)). The purified fraction was lyophilized overnight. There was [(1S)-2-[[2-(2,5-dioxopyrrol-1-yl)acetyl]amino]-1-methyl-ethyl]-4-[5-[3-[2-(4-tert-butoxy-4-oxo-butanoyl)-6-methoxy-isoindolin-5-yl]oxypropoxy]-4-fluoro-6-methoxy-benzothiophen-2-yl]-4-oxo-butanoate (0.065 g, 45.4703% yield) obtained as an off-white solid. LCMS: (ESI, m/z): [M+H]⁺=854.300.

### Step h:

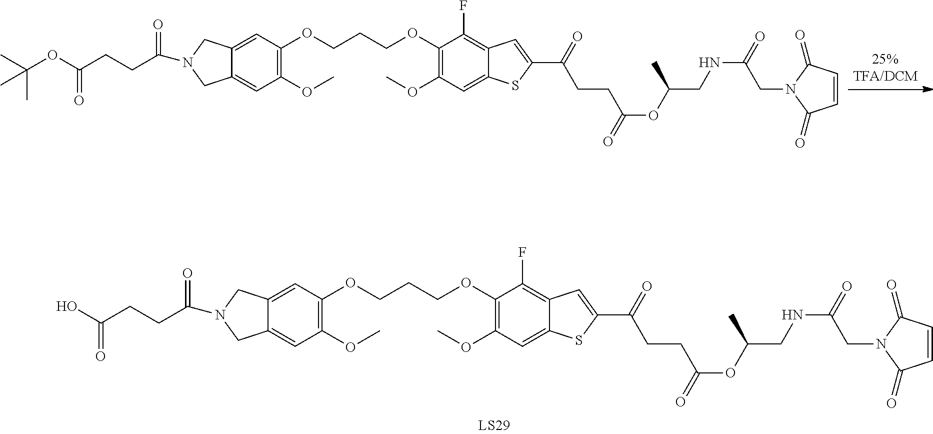 [SMILES: COc1cc2c(cc1OCCCOc1c(OC)cc3sc(C(=O)CCC(=O)O[C@@H](C)CNC(=O)CN4C(=O)C=CC4=O)cc3c1F)CN(C(=O)CCC(=O)OC(C)(C)C)C2 ; COc1cc2c(cc1OCCCOc1c(OC)cc3sc(C(=O)CCC(=O)O[C@@H](C)CNC(=O)CN4C(=O)C=CC4=O)cc3c1F)CN(C(=O)CCC(=O)O)C2]

[(1S)-2-[[2-(2,5-dioxopyrrol-1-yl)acetyl]amino]-1-methyl-ethyl]4-[5-[3-[2-(4-tert-butoxy-4-oxo-butanoyl)-6-methoxy-isoindolin-5-yl]oxypropoxy]-4-fluoro-6-methoxy-benzothiophen-2-yl]-4-oxo-butanoate (0.064 g, 74.9499 mol) was dissolved in 25% TFA (5 mL) in DCM at 25° C. The reaction mixture was stirred for 0.5 h at 25° C. The reaction mixture was concentrated under reduced pressure. The residue was was lyophilized overnight. The crude was further purified by prep-HPLC (the mobile phase: ACN/0.1% TFA-H₂O(0-50%)). The purified fraction was lyophilized overnight. There was 4-[5-[3-[2-[4-[(1S)-2-[[2-(2,5-dioxopyrrol-1-yl)acetyl]amino]-1-methyl-ethoxy]-4-oxo-butanoyl]-4-fluoro-6-methoxy-benzothiophen-5-yl]oxypropoxy]-6-methoxy-isoindolin-2-yl]-4-oxo-butanoic acid (0.0471 g, 78.7693% yield) obtained as a white solid. LCMS: (ESI, m/z): [M+H]⁺=798.250. ¹H NMR (400 MHz, DMSO-d₆) δ 12.07 (s, 1H), 8.32 (s, 1H), 8.26 (t, J=5.8 Hz, 1H), 7.57 (d, J=3.1 Hz, 1H), 7.08 (s, 2H), 6.99 (s, 0.5H), 6.96 (s, 1H), 6.93 (s, 0.5H), 4.88-4.79 (m, 1H), 4.74 (s, 2H), 4.53 (s, 2H), 4.30-4.14 (m, 4H), 4.04 (s, 2H), 3.87 (s, 3H), 3.73 (s, 3H), 3.41-3.34 (m, 4H), 3.26-3.15 (m, 2H), 2.68-2.62 (m, 2H), 2.60-2.55 (m, 2H), 2.18-2.08 (m, 2H), 1.11 (d, J=6.4 Hz, 3H).

### Example IV-30 Synthesis of Linker-STING Agonist Compound LS30

### Step a:

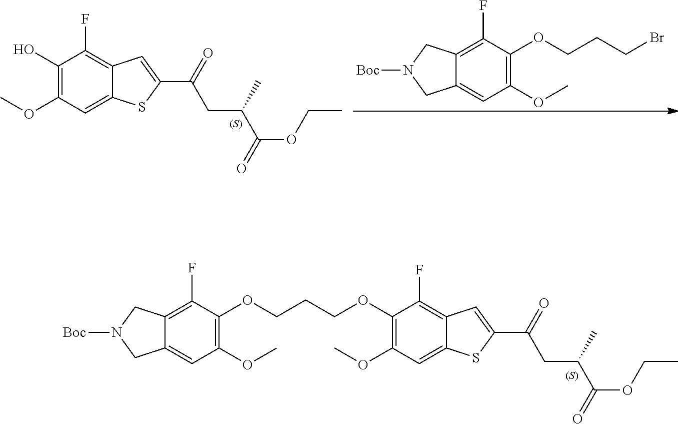

K₂CO₃ (0.51 g, 3.6902 mmol) was added to a solution of ethyl (S)-4-(4-fluoro-5-hydroxy-6-methoxybenzo[b]thiophen-2-yl)-2-methyl-4-oxobutanoate (0.45 g, 1.3221 mmol) and tert-butyl 5-(3-bromopropoxy)-4-fluoro-6-methoxyisoindoline-2-carboxylate (0.71 g, 1.7563 mmol) in N,N-Dimethylformamide (10 mL) at 25° C. under N₂ atmosphere. The reaction mixture was stirred at 60° C. for 3 h under N₂ atmosphere. The reaction mixture was concentrated and diluted with EA (100 mL), washed with NaHCO₃ aq(2×100 mL) and brine (50 mL). The organics were dried over Na₂SO₄, filtered and evaporated under reduced pressure. The residue was purified on silica gel column PE/EA (0-20%). The purified fraction was concentrated and dried under vacuo. There was tert-butyl 5-[3-[2-[(3S)-4-ethoxy-3-methyl-4-oxo-butanoyl]-4-fluoro-6-methoxy-benzothiophen-5-yl]oxypropoxy]-4-fluoro-6-methoxy-isoindoline-2-carboxylate (0.74 g, 1.1149 mmol, 84.3291% yield) obtained as a yellow solid. LCMS: (ESI, m/z): [M+H]⁺=664.231.

### Step b:

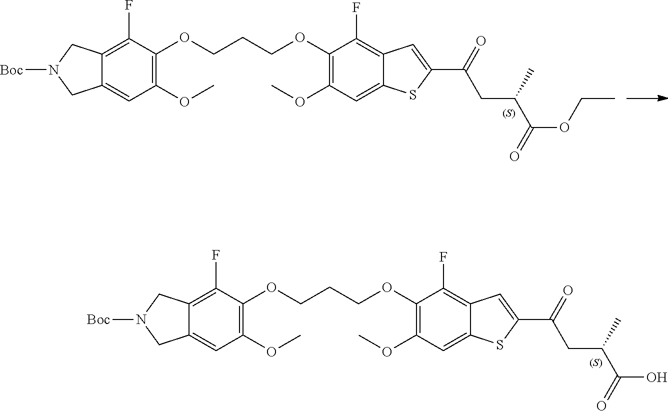

LiOH (0.05 g, 2.0878 mmol) was added to a solution of tert-butyl 5-[3-[2-[(3S)-4-ethoxy-3-methyl-4-oxo-butanoyl]-4-fluoro-6-methoxy-benzothiophen-5-yl]oxypropoxy]-4-fluoro-6-methoxy-isoindoline-2-carboxylate (0.74 g, 1.1149 mmol) in Tetrahydrofuran (12 mL) and Water (3 mL) at 25° C. The reaction mixture was stirred at 25° C. The reaction mixture was concentrated and diluted with EA (100 mL), washed with HCl (2×50 mL, 1M). The organics dried over Na₂SO₄, filtered and evaporated under reduced pressure. There was (2S)-4-[5-[3-(2-tert-butoxycarbonyl-4-fluoro- 6-methoxy-isoindolin-5-yl)oxypropoxy]-4-fluoro-6-methoxy-benzothiophen-2-yl]-2-methyl-4-oxo-butanoic acid (0.76 g, 1.1956 mmol, 107.2351% yield, Lot:20220512) obtained as a yellow solid. LCMS: (ESI, m/z): [M+H]⁺=636.200.

### Step c:

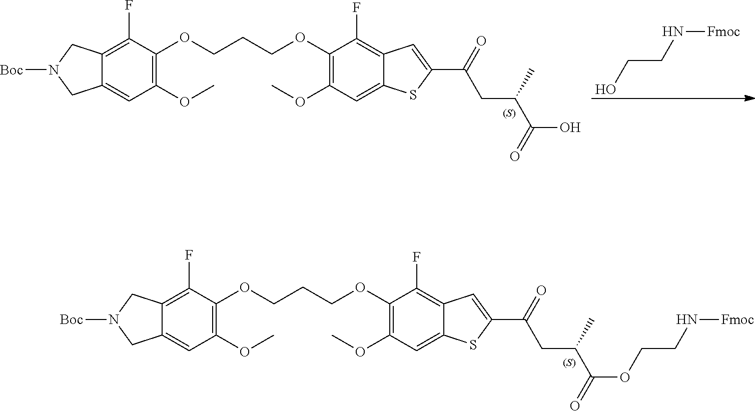

EDCI (0.52 g, 2.7126 mmol) was added to a solution of (2S)-4-[5-[3-(2-tert-butoxycarbonyl-4-fluoro-6-methoxy-isoindolin-5-yl)oxypropoxy]-4-fluoro-6-methoxy-benzothiophen-2-yl]-2-methyl-4-oxo-butanoic acid (0.71 g, 1.1169 mmol), Fmoc-Glycinol (0.90 g, 3.1766 mmol) and N-(4-pyridyl)dimethylamine (0.16 g, 1.3097 mmol) in dichloromethane (10 mL) at 25° C. under N₂ atmosphere. The reaction mixture was stirred at 40° C. under N₂ atmosphere. The reaction mixture was concentrated and diluted with EA (100 mL), washed with NaHCO₃ aq(2×500 mL) and brine (50 mL). The organics dried over Na₂SO₄, filtered and evaporated under reduced pressure. The residue was purified on silica gel column EA/PE (0-20%). The purified fraction was concentrated and dried under vacuo. There was tert-butyl-5-[3-[2-[(3S)-4-[2-(9H-fluoren-9-ylmethoxycarbonylamino)ethoxy]-3-methyl-4-oxo-butanoyl]-4-fluoro-6-methoxy-benzothiophen-5-yl]oxypropoxy]-4-fluoro-6-methoxy-isoindoline-2-carboxylate (1.03 g, 1.1432 mmol, 102.3523% yield) obtained as a yellow solid. LCMS: (ESI, m/z): [M+H]⁺=901.310.

### Step d:

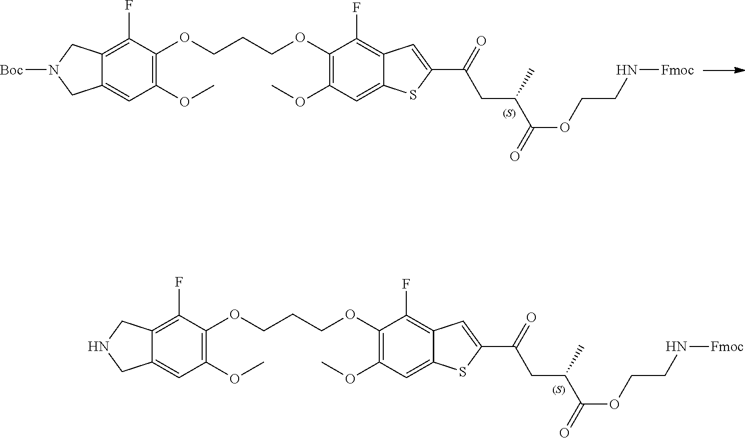

HCl (5 mL, 4M in EA) was added to a solution of tert-butyl 5-[3-[2-[(3S)-4-[2-(9H-fluoren-9-ylmethoxycarbonylamino)ethoxy]-3-methyl-4-oxo-butanoyl]-4-fluoro-6-methoxy-benzothiophen-5-yl]oxypropoxy]-4-fluoro-6-methoxy-isoindoline-2-carboxylate (1.03 g, 1.1432 mmol) in EA (10 mL) at 25° C. The reaction mixture was stirred at 25° C. . The residue was purified on C-18 column MeCN/water(0-50%). The purified fraction was concentrated and dried under vacuo. There was 2-(9H-fluoren-9-ylmethoxycarbonylamino)ethyl-(2S)-4-[4-fluoro-5-[3-(4-fluoro-6-methoxy-isoindolin-5-yl)oxypropoxy]-6-methoxy-benzothiophen-2-yl]-2-methyl-4-oxo-butanoate (0.80 g, 998.9240 mol, 87.3794% yield) obtained as a white solid. LCMS: (ESI, m/z): [M+H]⁺=801.258.

### Step e:

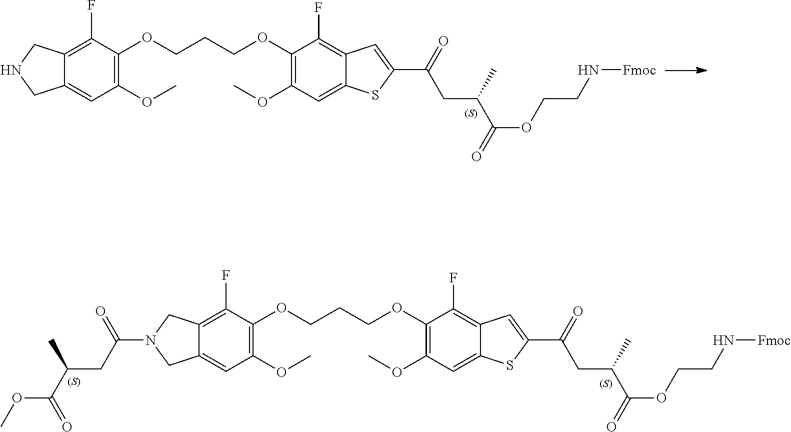

HATU (0.581 g, 1.5280 mmol) was added to a solution of N,N-diisopropylethylamine (0.624 g, 4.8281 mmol), 2-(9H-fluoren-9-ylmethoxycarbonylamino)ethyl (2S)-4-[4-fluoro-5-[3-(4-fluoro-6-methoxy-isoindolin-5-yl)oxypropoxy]-6-methoxy-benzothiophen-2-yl]-2-methyl-4-oxo-butanoate (0.800 g, 998.9240 μmol) and (S)-2-methyl-Butanedioic acid-1-methyl ester (0.312 g, 2.1349 mmol) in DMF (10 mL) at 25° C. The reaction mixture was stirred at 25° C. for 2 h. The reaction mixture was concentrated and diluted with EA (100 mL), washed with NaHCO₃ aq. (2×100 mL) and brine (50 mL). The organics were dried over Na₂SO₄, filtered and evaporated under reduced pressure. The residue was purified on silica gel column EA/PE (0-50%). The purified fraction was concentrated and dried under vacuo. There was 2-(9H-fluoren-9-ylmethoxycarbonylamino)ethyl(2S)-4-[4-fluoro-5-[3-[4-fluoro-6-methoxy-2-[(3S)-4-methoxy-3-methyl-4-oxo-butanoyl]isoindolin-5-yl]oxypropoxy]-6-methoxy-benzothiophen-2-yl]-2-methyl-4-oxo-butanoate (0.49 g, 527.4561 mol, 52.8024% yield) obtained as a yellow solid. LCMS: (ESI, m/z): [M+H]⁺=929.305.

### Step f:

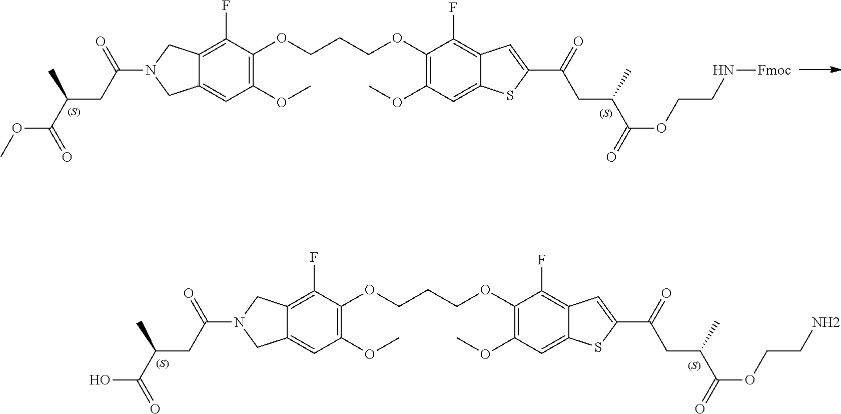

LiOH, (0.030 g, 1.2527 mmol) was added to a solution of 2-(9H-fluoren-9-ylmethoxycarbonylamino)ethyl-(2S)-4-[4-fluoro-5-[3-[4-fluoro-6-methoxy-2-[(3S)-4-methoxy-3-methyl-4-oxo-butanoyl]isoindolin-5-yl]oxypropoxy]-6-methoxy-benzothiophen-2-yl]-2-methyl-4-oxo-butanoate (0.452 g, 486.5513 mol) in tetrahydrofuran (10 mL) and water (2.5 mL) at 25° C. The reaction mixture was stirred at 25° C. for 2 h. The reaction mixture was concentrated and diluted with EA (100 mL), washed with HCl aq(2×50 mL, 1M). The organics were dried over Na₂SO₄, filtered and evaporated under reduced pressure. The residue was purified on C-18 column MeCN/water (0-100%). The purified fraction was concentrated and dried under vacuo. There was (2S)-4-[5-[3-[2-[(3S)-4-(2-aminoethoxy)-3-methyl-4-oxo-butanoyl]-4-fluoro-6-methoxy-benzothiophen-5-yl]oxypropoxy]-4-fluoro-6-methoxy-isoindolin-2-yl]-2-methyl-4-oxo-butanoic acid (64 mg, 92.3891 mol, 18.9886% yield) obtained as yellow solid. LCMS: (ESI, m/z): [M+H]⁺=693.222.

### Step g:

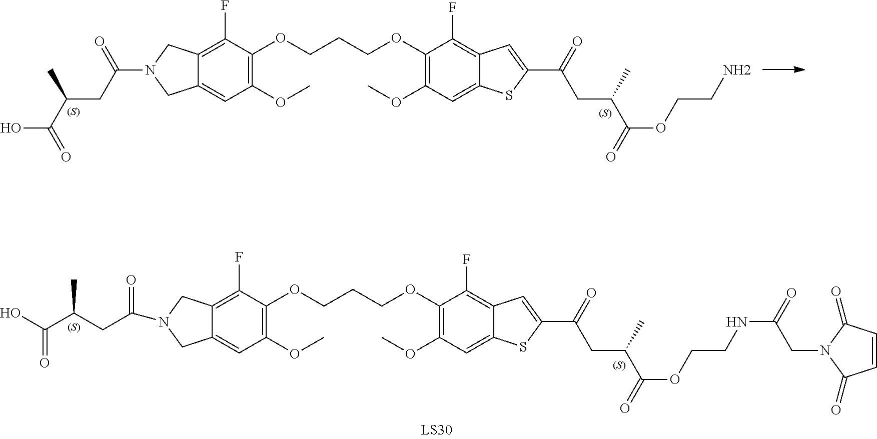

N-succinimidyl maleimidoacetate (0.026 g, 103.1010 mol) was added to a solution of (2S)-4-[5-[3-[2-[(3S)-4-(2-aminoethoxy)-3-methyl-4-oxo-butanoyl]-4-fluoro-6-methoxy-benzothiophen-5-yl]oxypropoxy]-4-fluoro-6-methoxy-isoindolin-2-yl]-2-methyl-4-oxo-butanoic acid (0.064 g, 92.3892 mol) in DCM (10 mL) at 0° C. under N₂ atmosphere. 4-Methylmorpholine (0.048 g, 474.5568 mol) was added to above reaction solution. The reaction mixture was stirred at 0° C. for 20 mins. The residue was purified by Pre-HPLC. The reaction mixture was quenched with adding of HCl (0.5 mL, 1 M) at 0° C. There was (2S)-4-[5-[3-[2-[(3S)-4-[2-[[2-(2,5-dioxopyrrol-1-yl)acetyl]amino]ethoxy]-3-methyl-4-oxo-butanoyl]-4-fluoro-6-methoxy-benzothiophen-5-yl]oxypropoxy]-4-fluoro-6-methoxy-isoindolin-2-yl]-2-methyl-4-oxo-butanoic acid (0.0112 g, 13.4970 mol, 14.6088% yield) obtained as a white solid. LCMS: (ESI, m/z): [M+H]⁺=830.233. ¹H NMR (400 MHz, DMSO-d₆) δ 8.32 (s, 1H), 8.27 (s, 1H), 7.57 (s, 1H), 7.07 (s, 2H), 6.88 (d, J=18.5 Hz, 1H), 4.78 (s, 2H), 4.56 (d, J=6.7 Hz, 2H), 4.28-4.20 (m, 4H), 4.10-3.95 (m, 4H), 3.88 (s, 3H), 3.77 (s, 3H), 2.78-2.66 (m, 2H), 2.10-2.04 (m, 2H), 1.29-1.16 (m, 3H), 1.17-1.04 (m, 3H).

### Example IV-31 Synthesis of Linker-STING Agonist Compound LS31

### Step a:

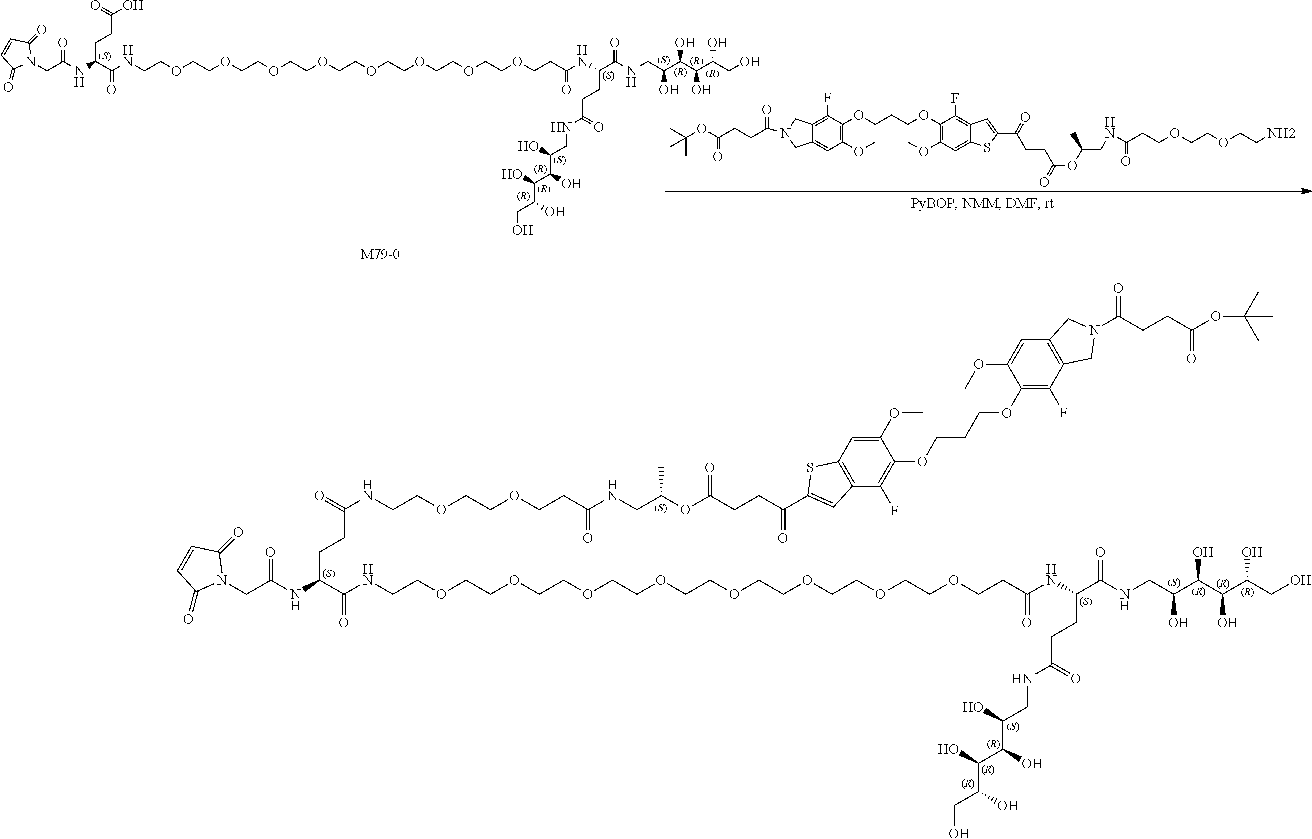

(S)-1-(3-(2-(2-aminoethoxy)ethoxy)propanamido)propan-2-yl-4-(5-(3-((2-(4-(tert-butoxy)-4-oxobutanoyl)-4-fluoro-6-methoxyisoindolin-5-yl)oxy)propoxy)-4-fluoro-6-methoxybenzo[b]thiophen-2-yl)-4-oxobutanoate (41.3000 mg, 46.1977 mol) and NMM (0.0277 g, 273.8588 mol) was added to a solution of (4S,35S,41S,42R,43R,44R)-4-(2-(2,5-dioxo-2,5-dihydro-1H-pyrrol-1-yl)acetamido)-41,42,43,44,45-pentahydroxy-5,33,38-trioxo-35-(((2S,3R,4R,5R)-2,3,4,5,6-pentahydroxyhexyl)carbamoyl)-9,12,15,18,21,24,27,30-octaoxa-6,34,39-triazapentatetracontanoic acid (0.0546 g, 46.9405 mol) and PyBOP (0.0727 g, 139.7025 mol) in DMF (3 mL) at 20° C. under N₂ atmosphere. The reaction mixture was stirred for 2 h at 20° C. The mixture was purified on C18 column ACN/H₂O (0.1% TFA)(0-40%). The pure fraction was concentrated and dried under vacuo. There was [(1S)-2-[3-[2-[2-[[(4S)-4-[[2-(2,5-dioxopyrrol-1-yl)acetyl]amino]-5-oxo-5-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]pentanoyl]amino]ethoxy]ethoxy]propanoylamino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.08 g, 39.2321 mol, 84.9224% yield) obtained as a brown oil. LCMS: (ESI, m/z): [M+2H]²⁺=1020.450.

### Step b:

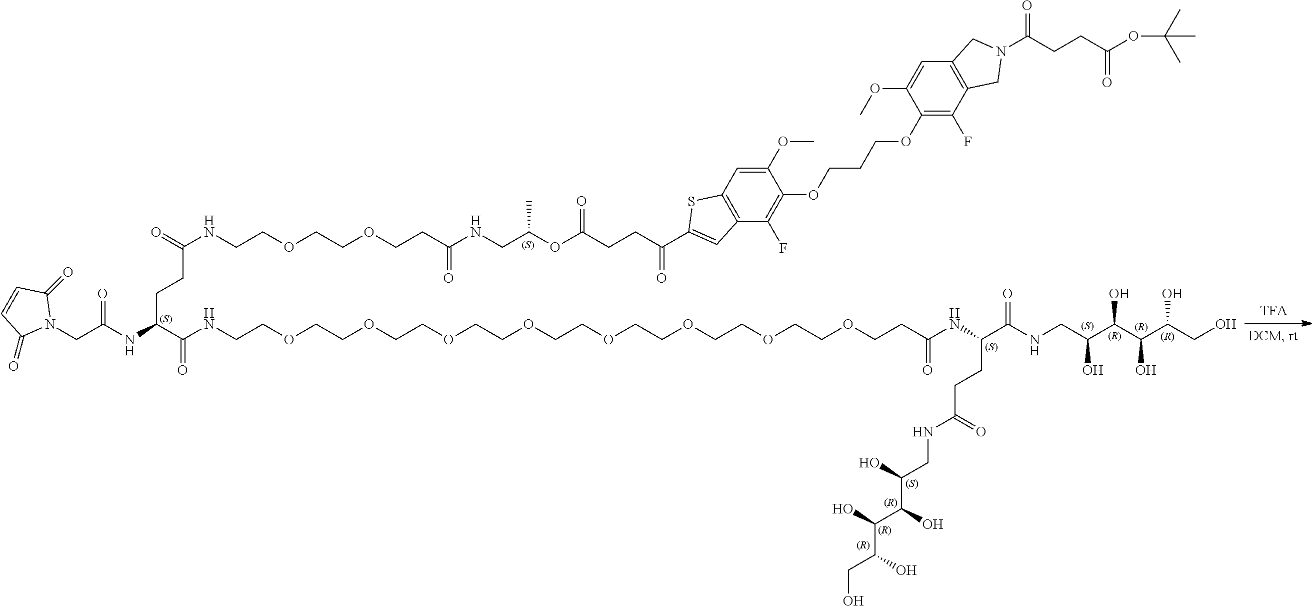

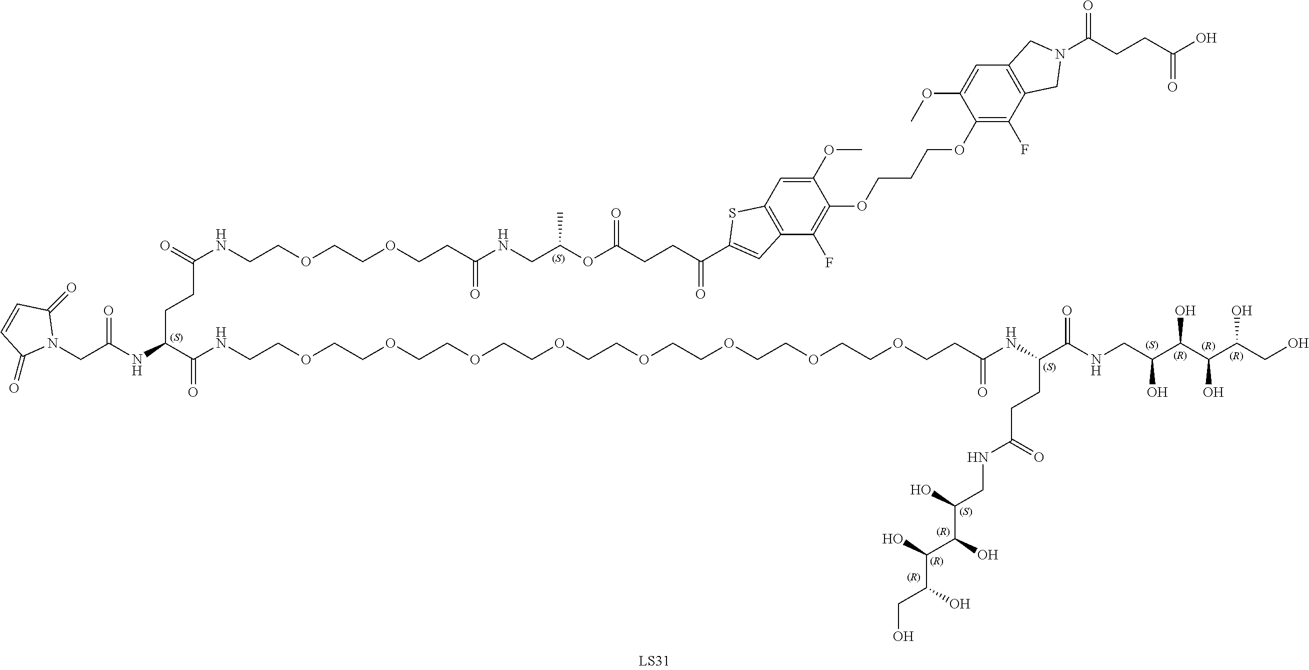

[(1S)-2-[3-[2-[2-[[(4S)-4-[[2-(2,5-dioxopyrrol-1-yl)acetyl]amino]-5-oxo-5-[2-[2-[2-[2-[2-[2-[2-[2-[3-oxo-3-[[(1S)-4-oxo-4-[[(2S,3R,4R,5R)-2,3,4,5,6-pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]pentanoyl]amino]ethoxy]ethoxy]propanoylamino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.08 g, 39.2322 mol) was added to a solution of TFA (1 mL) and DCM (3 mL) at 20° C. under open-air atmosphere. The reaction mixture was stirred for 1 h at 20° C. The reaction mixture was evaporated under reduced pressure. The residue was purified on Prep-HPLC. The pure fraction was concentrated and dried under vacuo. There was 4-[5-[3-[2-[4-[(1S)-2-[3-[2-[2-[[(4S)-4-[[2-(2,5-dioxopyrrol-1-yl)acetyl]amino]-5- oxo-5-[2-[2-[2-[2-[2-[2-[2-[2-[3-oxo-3-[[(1S)-4-oxo-4-[[(2S,3R,4R,5R)-2,3,4,5,6- pentahydroxyhexyl]amino]-1-[[(2S,3R,4R,5R)-2,3,4,5,6-pentahydroxyhexyl]carbamoyl]butyl]amino]propoxy]ethoxy]ethoxy]ethoxy]ethoxy]ethoxy]ethoxy]ethoxy]ethylamino]pentanoyl]amino]ethoxy]ethoxy]propanoylamino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (20.400 mg, 10.2872 mol, 26.2214% yield) obtained as a white solid. LCMS: (ESI, m/z): [M+2H]²⁺=992.450.

### Example IV-32 Synthesis of Linker-STING Agonist Compound LS32

### Step a:

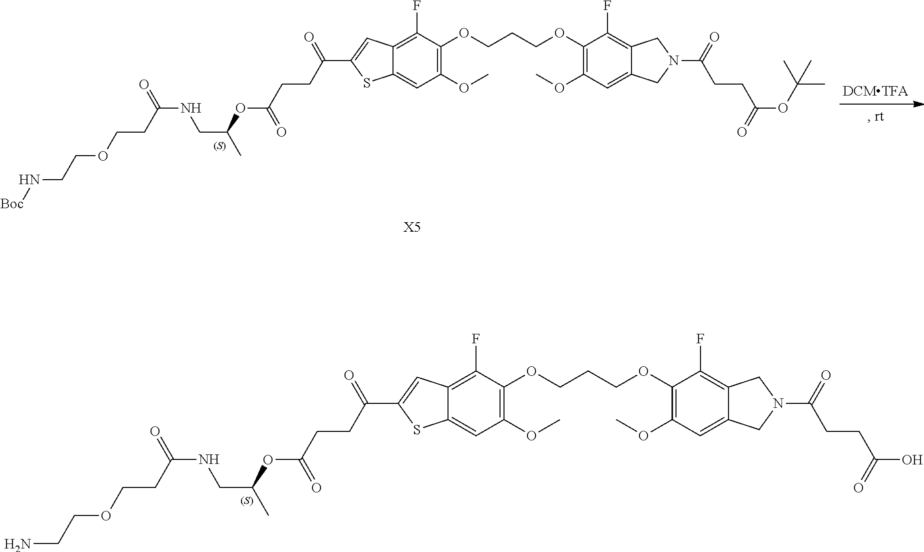

Trifluoroacetic acid (1 mL) was added to a solution of tert-butyl (S)-4-(4-fluoro-5-(3-((4-fluoro-6-methoxy-2-(2,2,14-trimethyl-4,11,16-trioxo-3,8,15-trioxa-5,12-diazanonadecan-19-oyl)benzo[b][thiophen-5-yl)oxy) propoxy)-6-methoxyisoindolin-2-yl)-4-oxobutanoate (X5, 0.153 g, 161.0446 mol) in DCM (5 mL) at 25° C., wherein X₅ was synthesized through the method as described in Example IV-27. The reaction mixture was stirred for 1 h at 25° C., and then evaporated under reduced pressure. The residue was purified on C18 column ACN/H₂O (0.1% TFA)(0-40%). The pure fraction was concentrated and dried by lyohpilization.

There was 4-[5-[3-[2-[4-[(1S)-2-[3-(2-aminoethoxy)propanoylamino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (0.099 g, 124.7125 mol, 77.4397% yield) obtained as an off-white solid. LCMS: (ESI, m/z): [M+H]⁺=794.270.

### Step b:

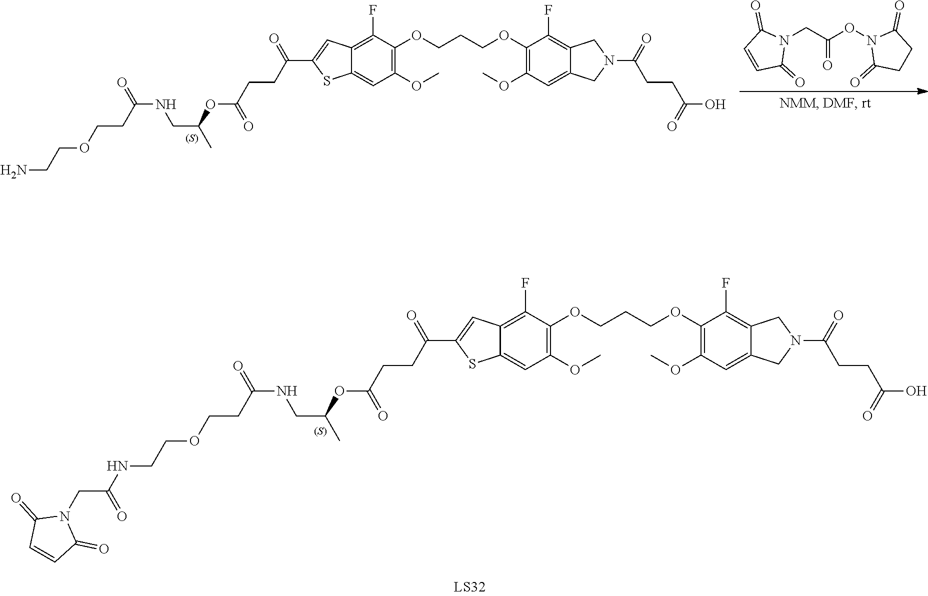

NMM (0.045 g, 444.8970 mol) was added to a solution of 4-[5-[3-[2-[4-[(1S)-2-[3-(2-aminoethoxy)propanoylamino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (0.099 g, 124.7125 mol) and N-Succinimidyl maleimidoacetate (0.063 g, 249.8216 mol) in DMF (3 mL) at 25° C. under open-air atmosphere. The reaction mixture was stirred for 2 h at 25° C., and then was purified on C18 column ACN/H₂O (0.1% TFA)(0-70%). The pure fraction was concentrated and dried by lyophilization. The residue was purified on Prep-HPLC, and then concentrated and dried by lyophilization. There was 4-[5-[3-[2-[4-[(1S)-2-[3-[2-[[2-(2,5-dioxopyrrol-1-yl)acetyl]amino]ethoxy]propanoylamino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (0.0528 g, 56.7182 mol, 45.4791% yield) obtained as a white solid.

LCMS: (ESI, m/z): [M+H]⁺=931.280. ¹H NMR (600 MHz, DMSO-d₆) δ 12.08 (s, 1H), 8.31 (s, 1H), 8.19 (t, J=5.6 Hz, 1H), 7.95 (t, J=6.0 Hz, 1H), 7.57 (d, J=3.8 Hz, 1H), 7.08 (s, 2H), 6.90 (s, 0.5H), 6.87 (s, 0.5H), 4.87-4.80 (m, 1H), 4.79 (s, 2H), 4.56 (d, J=9.6 Hz, 2H), 4.29-4.23 (m, 2H), 4.20 (t, J=6.0 Hz, 2H), 4.02 (s, 2H), 3.89 (s, 3H), 3.78 (d, J=3.5 Hz, 3H), 3.60 (t, J=6.5 Hz, 2H), 3.39-3.34 (m, 4H), 3.26-3.14 (m, 4H), 2.71-2.54 (m, 4H), 2.49-2.45 (m, 2H), 2.37-2.32 (m, 2H), 2.09-2.01 (m, 2H), 1.12 (d, J=6.3 Hz, 3H).

### Example IV-33 Synthesis of Linker-STING Agonist Compound LS33

### Step a:

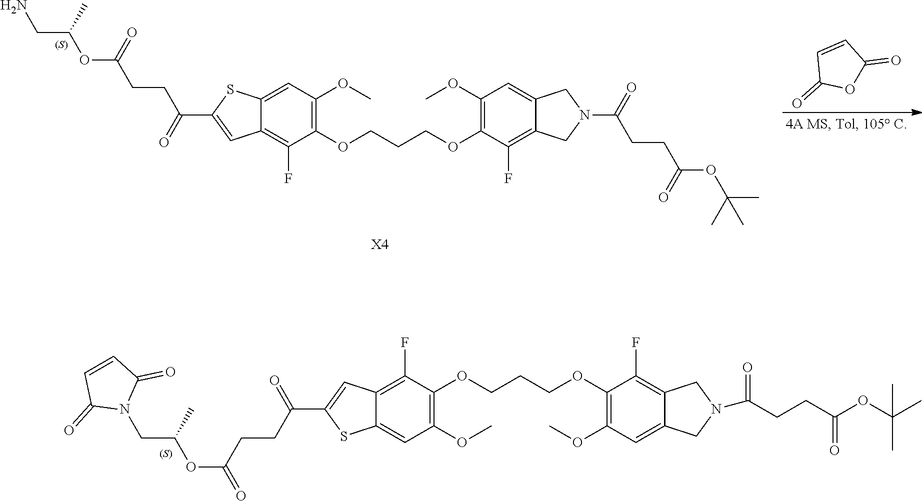

Furan-2,5-dione (0.260 g, 2.6515 mmol) and 4A MS (50 mg) was added to a mixture of (S)-1-aminopropan-2-yl-4-(5-(3-((2-(4-(tert-butoxy)-4-oxobutanoyl)-4-fluoro-6-methoxyisoindolin-5-yl)oxy)propoxy)-4-fluoro-6-methoxybenzo[b]thiophen-2-yl)-4-oxobutanoate (X₄, 0.192 g, 261.2949 mol) in toluene (5 mL) at 25° C. under N₂ atmosphere, wherein X₄ was synthesized through the method as described in Example IV-5. The reaction mixture was heated to 105° C. and stirred for 4 d. The precipitate was collected by filtration. The filtrate was evaporated under reduced pressure. The residue was purified on C18 column ACN/H₂O (0.1% TFA)(0-60%). The pure fraction was concentrated and dried under vacuo. There was [(1S)-2-(2,5-dioxopyrrol-1-yl)-1-methyl-ethyl]4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.04 g, 49.0892 mol, 18.7869% yield) obtained as a brown solid. LCMS: (ESI, m/z): [M+H]⁺=815.260.

### Step b:

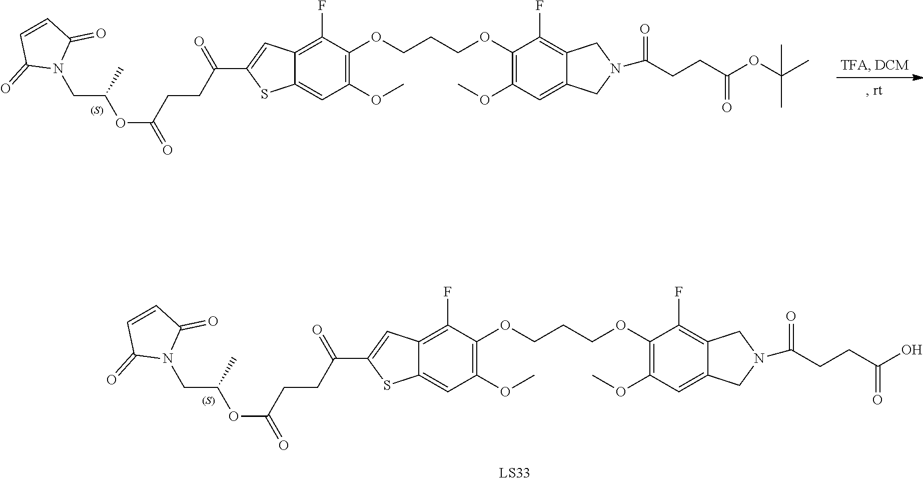

[(1S)-2-(2,5-dioxopyrrol-1-yl)-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.040 g, 49.0892 mol) was added to a solution of TFA (1 mL) and DCM (4 mL) at 25° C. under open-air atmosphere. The reaction mixture was stirred at 25° C., and then evaporated under reduced pressure. The residue was purified on Prep-HPLC. The pure fraction was dried by lyophilization. There was 4-[5-[3-[2-[4-[(1S)-2-(2,5-dioxopyrrol-1-yl)-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (0.0103 g, 13.5752 mol, 27.6541% yield ) obtained as a yellow solid. LCMS: (ESI, m/z): [M+H]⁺=759.200. ¹H NMR (400 MHz, DMSO-d₆) δ 12.10 (s, 1H), 8.28 (s, 1H), 7.56 (s, 1H), 7.01 (s, 2H), 6.90 (s, 0.5H), 6.87 (s, 0.5H), 5.05-4.94 (m, 1H), 4.79 (s, 2H), 4.56 (d, J=6.2 Hz, 2H), 4.31-4.23 (m, 2H), 4.20 (t, J=6.1 Hz, 2H), 3.89 (s, 3H), 3.78 (d, J=2.2 Hz, 3H), 3.60-3.50 (m, 2H), 3.31 (t, J=6.4 Hz, 2H), 2.64-2.54 (m, 6H), 2.10-1.99 (m, 2H), 1.15 (d, J=6.4 Hz, 3H).

### Example IV-34 Synthesis of Linker-STING Agonist Compound LS34

### Step a:

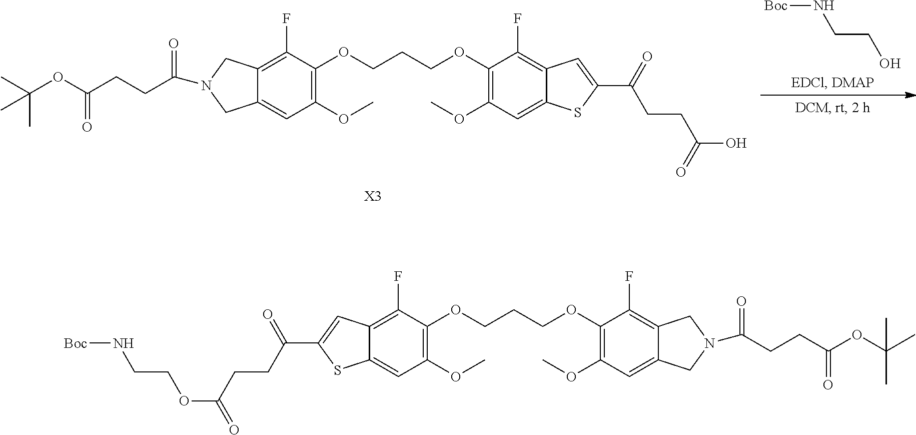 [SMILES: COc1cc2c(c(F)c1OCCCOc1c(OC)cc3sc(C(=O)CCC(=O)O)cc3c1F)CN(C(=O)CCC(=O)OC(C)(C)C)C2 ; CC(C)(C)OC(=O)NCCO ; "X3" ; COc1cc2c(c(F)c1OCCCOc1c(OC)cc3sc(C(=O)CCC(=O)OCCNC(=O)OC(C)(C)C)cc3c1F)CN(C(=O)CCC(=O)OC(C)(C)C)C2]

4-(5-(3-((2-(4-(tert-butoxy)-4-oxobutanoyl)-4-fluoro-6-methoxyisoindolin-5-yl)oxy)propoxy)-4-fluoro-6-methoxybenzo[b]thiophen-2-yl)-4-oxobutanoic acid (0.342 g, 504.6422 mol) was added to a solution of EDCI (0.298 g, 1.5545 mmol), N-(4-pyridyl)dimethylamine (X₃, 0.035 g, 286.4931 mol) in DCM (5 mL) at 25° C., wherein X₃ was synthesized through the method as described in Example IV-5. The reaction mixture was stirred for 2 h at 25° C. and then evaporated under reduced pressure. The residue was purified on C18 column MeCN/Water (0-65%). The pure fraction was concentrated and dried under vacuo, and then concentrated and dried by lyopilization at 25° C. There was 2-(tert-butoxycarbonylamino)ethyl 4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.253 g, 308.2017 mol, 61.0733% yield) obtained as a white solid. LCMS: (ESI, m/z): [M+H]⁺=821.305. ¹H NMR: (400 MHz, DMSO-d₆) δ 8.31 (s, 1H), 7.57 (s, 1H), 6.93 (t, J=5.6 Hz, 1H), 6.90 (s, 0.5H), 6.87 (s, 0.5H), 4.78 (s, 2H), 4.56 (d, J=7.6 Hz, 2H), 4.26 (t, J=5.9 Hz, 2H), 4.20 (t, J=6.0 Hz, 2H), 4.00 (t, J=5.6 Hz, 2H), 3.88 (s, 3H), 3.79 (s, 3H), 3.38 (t, J=6.3 Hz, 2H), 3.20-3.11 (m, 2H), 2.67 (t, J=6.2 Hz, 2H), 2.59-2.53 (m, 2H), 2.48-2.44 (m, 2H), 2.11-1.99 (m, 2H), 1.39 (d, J=8.4 Hz, 18H).

### Step b:

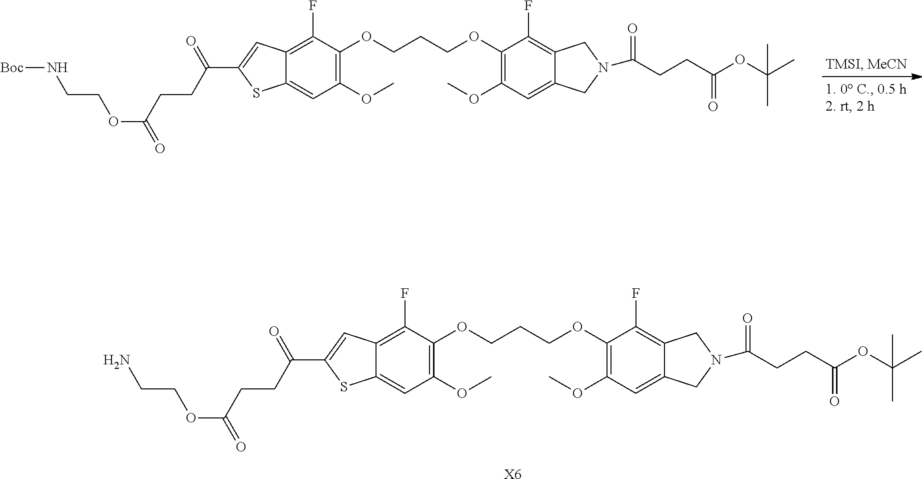 [SMILES: COc1cc2c(c(F)c1OCCCOc1c(OC)cc3sc(C(=O)CCC(=O)OCCNC(=O)OC(C)(C)C)cc3c1F)CN(C(=O)CCC(=O)OC(C)(C)C)C2 ; COc1cc2c(c(F)c1OCCCOc1c(OC)cc3sc(C(=O)CCC(=O)OCCN)cc3c1F)CN(C(=O)CCC(=O)OC(C)(C)C)C2 ; "X6"]

TMSI (0.091 g, 454.7885 mol) in MeCN (8 ml) was added to a solution of 2-(tert-butoxycarbonylamino)ethyl-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.250 g, 304.5471 mol) in MeCN (10 mL) at 0° C. The reaction mixture was stirred for 0.5 h at 0° C. The reaction mixture was stirred for 2 h at 25° C. The reaction mixture was quenched with water (2 mL), and then evaporated under reduced pressure. The resulting reaction mixture was purified on C18 column MeCN/Water (0-70%). The pure fraction was concentrated and dried under vacuo. There was 2-aminoethyl 4-(5-(3-((2-(4-(tert-butoxy)-4-oxobutanoyl)-4-fluoro-6-methoxyisoindolin-5-yl)oxy)propoxy)-4-fluoro-6-methoxybenzo[b]thiophen-2-yl)-4-oxobutanoate (0.160 g, 221.9831 mol, 72.8896% yield) obtained as a yellow solid. LCMS: (ESI, m/z): [M+H]⁺=721.253.

### Step c:

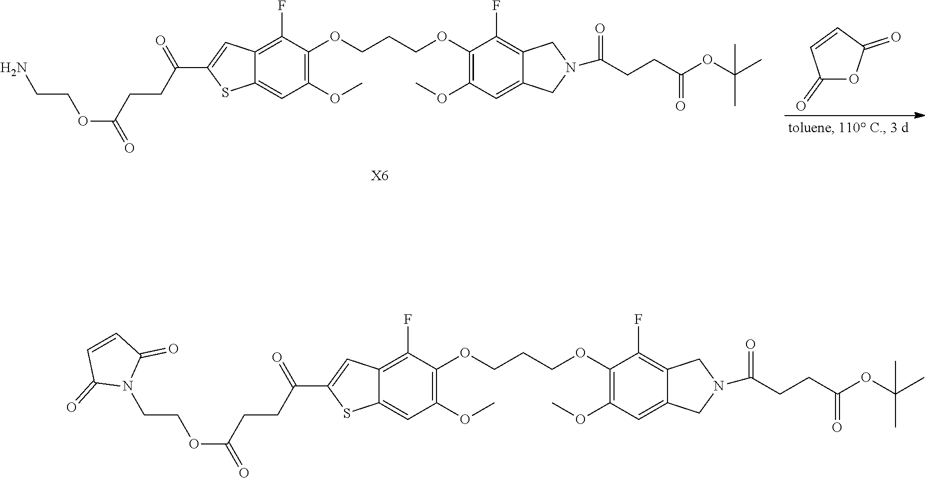 [SMILES: COc1cc2c(c(F)c1OCCCOc1c(OC)cc3sc(C(=O)CCC(=O)OCCN)cc3c1F)CN(C(=O)CCC(=O)OC(C)(C)C)C2 ; O=C1C=CC(=O)O1 ; "X6" ; COc1cc2c(c(F)c1OCCCOc1c(OC)cc3sc(C(=O)CCC(=O)OCCN4C(=O)C=CC4=O)cc3c1F)CN(C(=O)CCC(=O)OC(C)(C)C)C2]

2-aminoethyl 4-(5-(3-((2-(4-(tert-butoxy)-4-oxobutanoyl)-4-fluoro-6-methoxyisoindolin-5-yl)oxy)propoxy)-4-fluoro-6-methoxybenzo[b]thiophen-2-yl)-4-oxobutanoate (0.042 g, 58.2706 mol) was added to a solution of furan-2,5-dione (0.036 g, 367.1342 mol) in toluene (2 mL) at 25° C. The reaction mixture was stirred for 3 d at 110° cand then evaporated under reduced pressure. The residue was purified on C18 column MeCN/Water (0-65%). The pure fraction was concentrated and dried under vacuo. There was 2-(2,5-dioxopyrrol-1-yl)ethyl 4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.021 g, 26.2232 mol, 45.0025% yield) obtained as a yellow slod. LCMS: (ESI, m/z): [M+H]⁺=801.243.

### Step d:

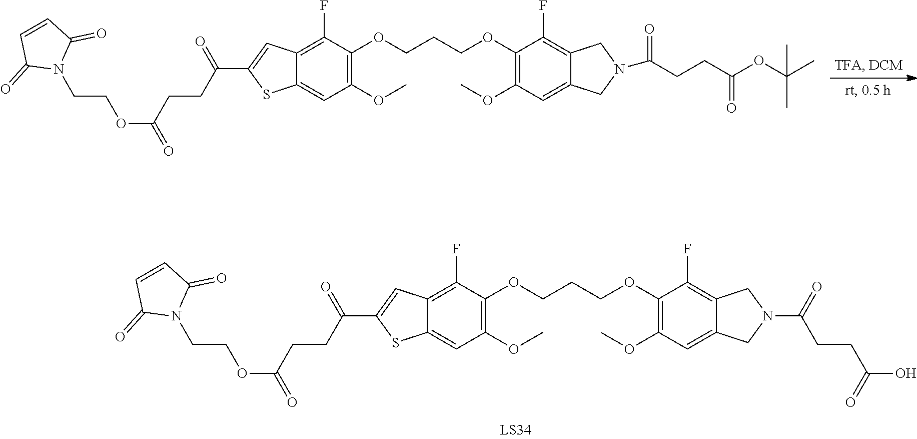 [SMILES: COc1cc2c(c(F)c1OCCCOc1c(OC)cc3sc(C(=O)CCC(=O)OCCN4C(=O)C=CC4=O)cc3c1F)CN(C(=O)CCC(=O)OC(C)(C)C)C2 ; COc1cc2c(c(F)c1OCCCOc1c(OC)cc3sc(C(=O)CCC(=O)OCCN4C(=O)C=CC4=O)cc3c1F)CN(C(=O)CCC(=O)O)C2]

To a stirred solution were added 2-(2,5-dioxopyrrol-1-yl)ethyl-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.021 g, 26.2232 mol), trifluoroacetic acid (0.4 mL) and DCM (2 mL) at 25° C. The reaction mixture was stirred for 0.5 h at 25° C., and then evaporated under reduced pressure. The residue was purified by Perp-HPLC. The pure fraction was concentrated and dried by lyopilization at 25° C. There was 4-[5-[3-[2-[4-[2-(2,5-dioxopyrrol-1-yl)ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (4.7 mg, 6.3112 mol, 24.0671% yield) obtained as a white solid. LCMS: (ESI, m/z): [M+H]⁺=745.180

### Example IV-35 Synthesis of Linker-STING Agonist Compound LS35

### Step a:

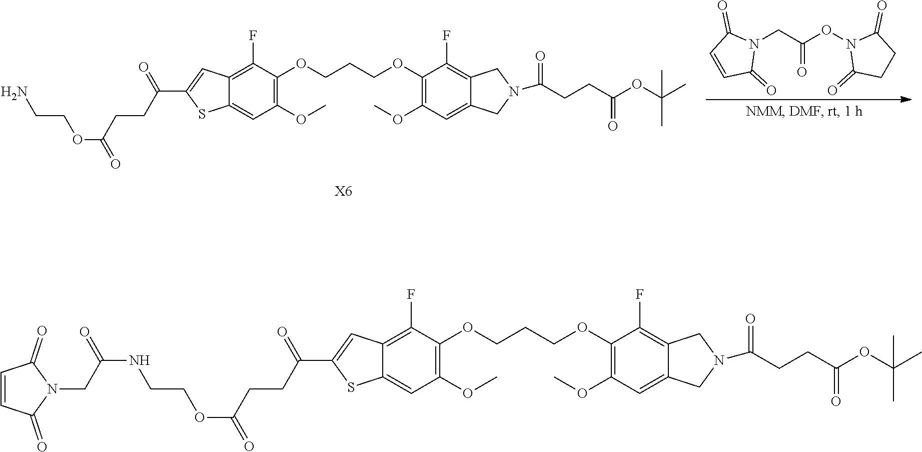

NMM (0.017 g, 168.0722 mol) was added to a solution of N-Succinimidyl maleimidoacetate (0.053 g, 210.1673 mol), 2-aminoethyl 4-(5-(3-((2-(4-(tert-butoxy)-4-oxobutanoyl)-4-fluoro-6-methoxyisoindolin-5-yl)oxy)propoxy)-4-fluoro-6-methoxybenzo[b]thiophen-2-yl)-4-oxobutanoate (0.063 g, 87.4058 mol) in DMF (2 mL) at 25° C. The reaction mixture was stirred for 1 h at 25° C. PH was adjusted to 5 with TFA. The reaction mixture was evaporated under vacuo pressure. The residue was purified on C18 column MeCN/Water (0-80%). The pure fraction was concentrated and dried under vacuo, and then concentrated and dried by lyohpilization at 25° C. There was 2-[[2-(2,5-dioxopyrrol-1-yl)acetyl]amino]ethyl 4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.043 g, 50.1242 mol, 57.3466% yield) obtained as an off-white solid. LCMS: (ESI, m/z): [M+H]⁺=858.264. ¹H NMR: (600 MHz, DMSO-d₆) δ 8.38-8.25 (m, 2H), 7.56 (d, J=4.1 Hz, 1H), 7.08 (s, 2H), 6.90 (s, 0.5H), 6.87 (s, 0.5H), 4.78 (s, 2H), 4.57 (s, 1H), 4.55 (s, 1H), 4.26 (t, J=5.2 Hz, 2H), 4.20 (t, J=5.9 Hz, 2H), 4.05-4.01 (m, 4H), 3.89 (s, 3H), 3.78 (d, J=2.1 Hz, 3H), 3.33-3.27 (m, 4H), 2.68 (t, J=6.3 Hz, 2H), 2.58-2.53 (m, 2H), 2.48-2.44 (m, 2H), 2.09-2.01 (m, 2H), 1.40 (s, 9H).

### Step b:

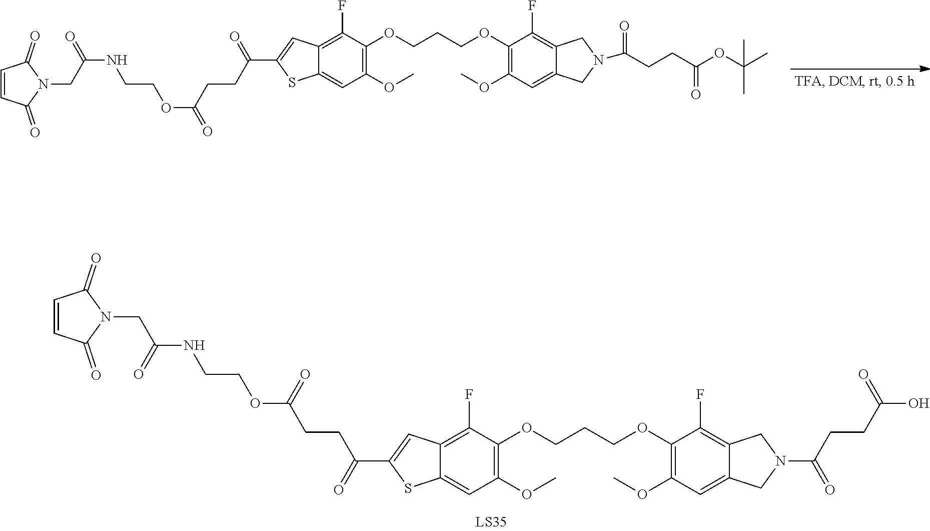 [SMILES: COc1cc2c(c(F)c1OCCCOc1c(OC)cc3sc(C(=O)CCC(=O)OCCNC(=O)CN4C(=O)C=CC4=O)cc3c1F)CN(C(=O)CCC(=O)OC(C)(C)C)C2 ; COc1cc2c(c(F)c1OCCCOc1c(OC)cc3sc(C(=O)CCC(=O)OCCNC(=O)CN4C(=O)C=CC4=O)cc3c1F)CN(C(=O)CCC(=O)O)C2]

To a stirred solution were added 2-[[2-(2,5-dioxopyrrol-1-yl)acetyl]amino]ethyl 4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.043 g, 50.1242 mol), trifluoroacetic acid, TFA (0.4 mL) and DCM (2 mL) at 25° C. The reaction mixture was stirred for 0.5 h at 25° C., and then evaporated under reduced pressure. The residue was purified by Perp-HPLC. The pure fraction was concentrated and dried by lyopilization at 25° C. There was 4-[5-[3-[2-[4-[2-[[2-(2,5-dioxopyrrol-1-yl)acetyl]amino]ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (20.0 mg, 24.9450 μmol, 49.7664% yield) obtained as a white solid. LCMS: (ESI, m/z): [M+H]- =802.202. ¹H NMR: (600 MHz, DMSO-d₆) δ 8.39-8.24 (m, 2H), 7.57 (d, J=3.8 Hz, 1H), 7.09 (s, 2H), 6.89 (d, J=17.8 Hz, 1H), 4.78 (s, 2H), 4.56 (d, J=10.7 Hz, 2H), 4.29-4.23 (m, 2H), 4.20 (t, J=5.8 Hz, 2H), 4.06-4.01 (m, 4H), 3.89 (s, 3H), 3.78 (d, J=3.3 Hz, 3H), 3.34-3.28 (m, 4H), 2.68 (t, J=6.3 Hz, 2H), 2.60-2.55 (m, 2H), 2.48 (d, J=6.7 Hz, 2H), 2.09-2.00 (m, 2H).

### Example IV-36 Synthesis of Linker-STING Agonist Compound LS36

### Step a:

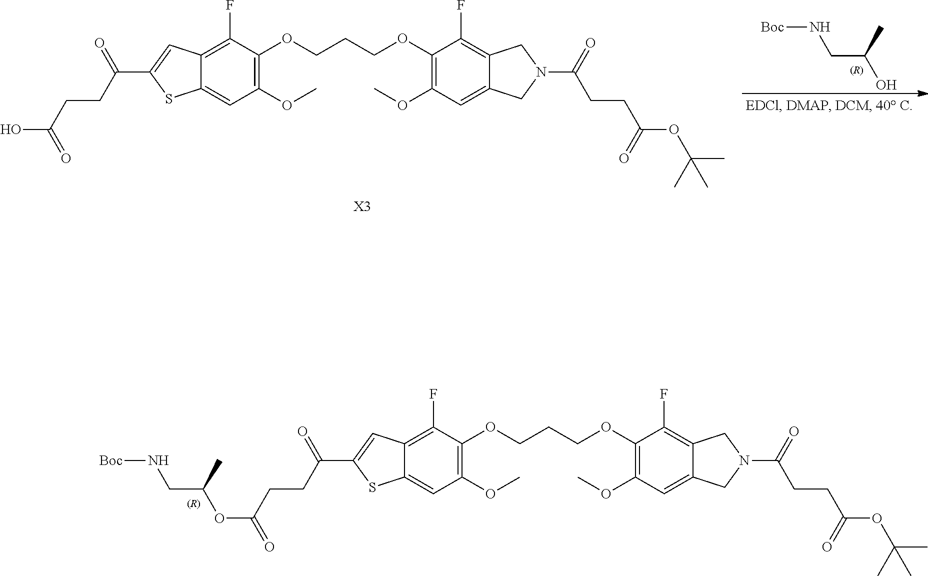

Tert-butyl (R)-(2-hydroxypropyl)carbamate (0.139 g, 793.2668 mol) was added to a mixture of 4-(5-(3-((2-(4-(tert-butoxy)-4-oxobutanoyl)-4-fluoro-6-methoxyisoindolin-5-yl)oxy)propoxy)-4-fluoro-6-methoxybenzo[b]thiophen-2-yl)-4-oxobutanoic acid (0.150 g, 221.3343 mol), DMAP(0.030 g, 245.5655 μmol) and EDCI (0.073 g, 380.8014 mol) in DCM (8 mL) at 25° C. under open-air atmosphere, wherein X₃ was synthesized through the method as described in Example IV-5. The reaction mixture was heated to 40° C. and stirred for 2 h, and then evaporated under reduced pressure. The residue was purified on C18 column ACN/H₂O (0.1% TFA)(0-70%). The pure fraction was concentrated and dried under vacuo. There was [(1R)-2-(tert-butoxycarbonylamino)-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.167 g, 200.0196 μmol, 90.3699% yield) obtained as a white solid. LCMS: (ESI, m/z): [M+H]- =835.320. ¹H NMR (600 MHz, DMSO-d₆) δ 8.30 (s, 1H), 7.56 (d, J=4.3 Hz, 1H), 6.94 (t, J=6.1 Hz, 1H), 6.90 (s, 0.5H), 6.87 (s, 0.5H), 4.82-4.76 (m, 3H), 4.57 (s, 1H), 4.55 (s, 1H), 4.26 (t, J=6.1, 2H), 4.20 (t, J=6.1, 2H), 3.89 (s, 3H), 3.78 (d, J=2.1 Hz, 3H), 3.37-3.35 (m, 2H), 3.13-2.98 (m, 2H), 2.71-2.53 (m, 4H), 2.50-2.44 (m, 2H), 2.09-2.01 (m, 2H), 1.40 (s, 9H), 1.38 (s, 9H), 1.10 (d, J=6.3 Hz, 3H).

### Step b:

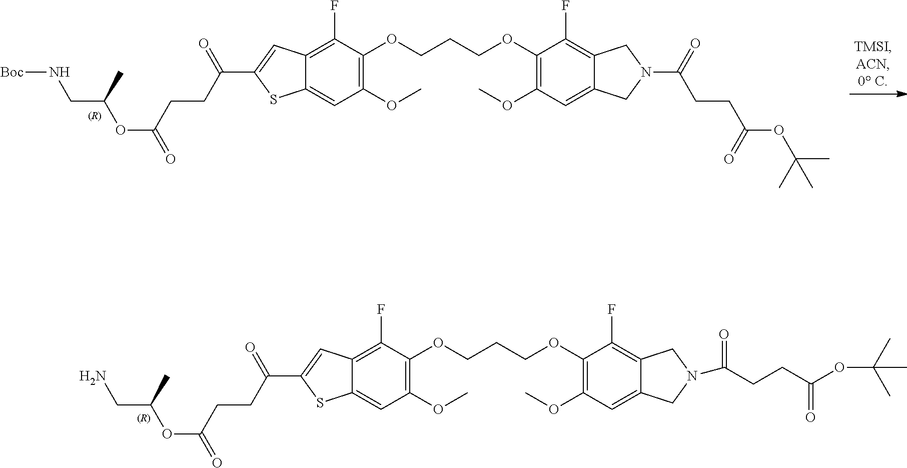

Iodotrimethylsilane (0.048 g, 239.8885 μmol) was added to a solution of [(1R)-2-(tert-butoxycarbonylamino)-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.149 g, 178.4607 μmol) in MeCN (5 mL) at 0° C. under N₂ atmosphere. The reaction mixture was stirred for 1.5 h at 25° C., and then quenched with addition of H₂O (10 mL) at 25° C. The mixture was purified on C18 column ACN/H₂O (0.1% TFA)(0-60%). The pure fraction was concentrated and dried dried by lyophilization. There was [(1R)-2-amino-i-methyl-ethyl]4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.078 g, 106.1510 mol, 59.4815% yield) obtained as a white solid. LCMS: (ESI, m/z): [M+H]⁺=735.270.

### Step c:

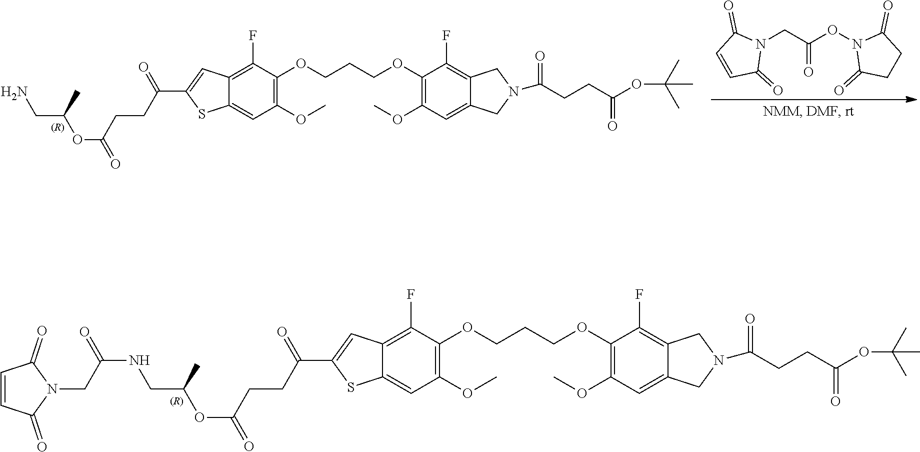

NMM (0.037 g, 365.8042 mol) was added to a solution of [(1R)-2-amino-1-methyl-ethyl]4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.077 g, 104.7901 mol) and N-Succinimidyl maleimidoacetate (0.061 g, 241.8907 mol) in DMF (3 mL) at 25° C. under N₂ atmosphere. The reaction mixture was stirred for 1 h at 25° C. The mixture was purified on C18 column ACN/H₂O (0.1% TFA)(0-70%). The pure fraction was concentrated and dried under vacuo. There was [(1R)-2-[[2-(2,5-dioxopyrrol-1-yl)acetyl]amino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo- butanoate (0.07 g, 80.2849 mol, 76.6150% yield) obtained as a white solid. LCMS: (ESI, m/z): [M+H]⁺=872.280.

### Step d:

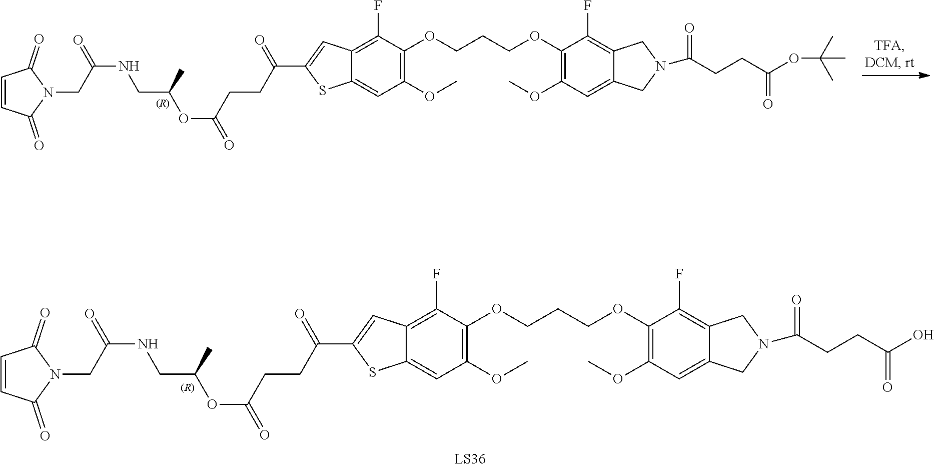

Trifluoroacetic acid (1 mL) was added to a solution of [(1R)-2-[[2-(2,5-dioxopyrrol-1-yl)acetyl]amino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.07 g, 80.2849 mol) in DCM (4 mL) at 25° C. under open-air atmosphere. The reaction mixture was stirred for 1 h at 25° C., and then was evaporated under reduced pressure. The residue was purified on Prep-HPLC. The pure fraction was concentrated and dried by lyophilization. There was 4-[5-[3-[2-[4-[(1R)-2-[[2-(2,5-dioxopyrrol-1-yl)acetyl]amino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (0.0334 g, 40.9420 mol, 50.9958% yield) obtained as a white solid. LCMS: (ESI, m/z): [M+H]⁺=816.220. ¹H NMR (600 MHz, DMSO-d₆) δ 12.09 (s, 1H), 8.31 (s, 1H), 8.26 (t, J=5.9 Hz, 1H), 7.57 (d, J=4.0 Hz, 1H), 7.08 (s, 2H), 6.90 (s, 0.5H), 6.87 (s, 0.5H), 4.86-4.80 (m, 1H), 4.79 (s, 2H), 4.57 (s, 1H), 4.56 (s, 1H), 4.25 (t, J=6.1, 2H), 4.20 (t, J=6.1, 2H), 4.04 (s, 2H), 3.89 (s, 3H), 3.78 (d, J=3.2 Hz, 3H), 3.46-3.37 (m, 2H), 3.26-3.17 (m, 2H), 2.69-2.63 (m, 2H), 2.61-2.55 (m, 2H), 2.50-2.46 (m, 2H), 2.09-2.01 (m, 2H), 1.11 (d, J=6.3 Hz, 3H).

### Example IV-37 Synthesis of Linker-STING Agonist Compound LS37

### Step a:

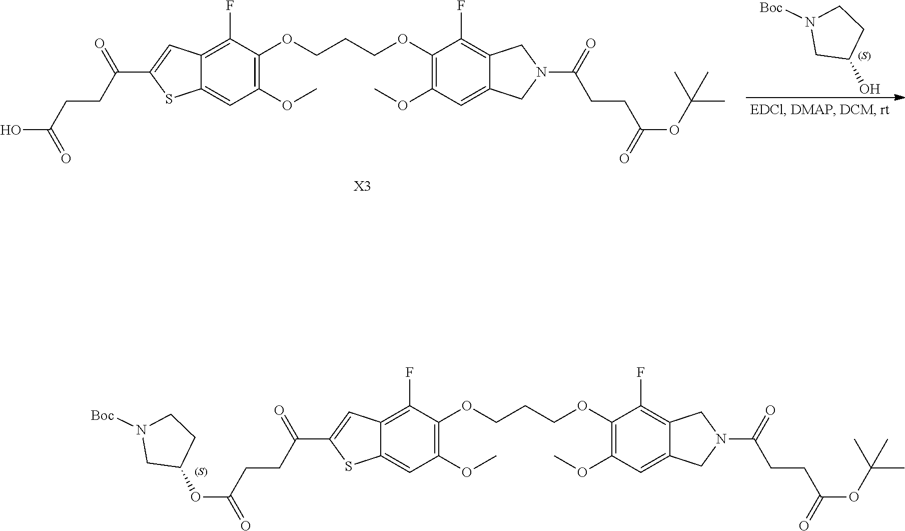

N-(tert-Butoxycarbonyl)-(S)-(+)-3-pyrrolidinol (0.170 g, 907.9475 mol) was added to a solution of 4-(5-(3-((2-(4-(tert-butoxy)-4-oxobutanoyl)-4-fluoro-6-methoxyisoindolin-5-yl)oxy)propoxy)-4-fluoro-6-methoxybenzo[b]thiophen-2-yl)-4-oxobutanoic acid (X₃, 0.164 g, 241.9922 mol), N-(4-pyridyl)dimethylamine (0.035 g, 286.4931 mol) and EDCI (0.073 g, 380.8014 mol) in DCM (8 mL) at 25° C. under open-air atmosphere, wherein X₃ was synthesized through the method as described in EXAMPLE IV-5. The reaction mixture was stirred for 2 h at 40° C., and then evaporated under reduced pressure. The residue was purified on C18 column ACN/H₂O (0.1% TFA)(0-70%). The pure fraction was concentrated and dried under vacuo. There was tert-butyl (3S)-3-[4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoyl]oxypyrrolidine-1-carboxylate (0.179 g, 211.3520 mol, 87.3384% yield) obtained as a white solid. LCMS: (ESI, m/z): [M+H]⁺=847.320. ¹H NMR (600 MHz, DMSO-d₆) δ 8.31 (s, 1H), 7.56 (d, J=4.7 Hz, 1H), 6.89 (s, 0.5H), 6.87 (s, 0.5H), 5.21-5.16 (m, 1H), 4.78 (s, 2H), 4.57 (s, 1H), 4.55 (s, 1H), 4.25 (t, J=6.2, 2H), 4.20 (t, J=6.1 Hz, 2H), 3.88 (s, 3H), 3.78 (d, J=1.9 Hz, 3H), 3.39-3.32 (m, 4H), 3.29-3.17 (m, 2H), 2.72-2.64 (m, 2H), 2.59-2.53 (m, 2H), 2.49-2.42 (m, 2H), 2.11-2.00 (m, 3H), 1.94-1.86 (m, 1H), 1.45-1.31 (m, 18H).

### Step b:

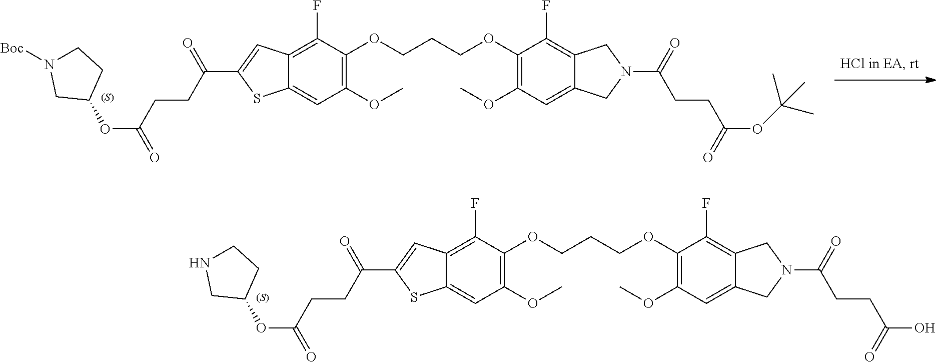

HCl in EA (5 mL, 4 M) was added to a solution of tert-butyl (S)-3-((4-(5-(3-((2-(4-(tert-butoxy)-4-oxobutanoyl)-4-fluoro-6-methoxyisoindolin-5-yl)oxy)propoxy)-4-fluoro-6-methoxybenzo [b]thiophen-2-yl)-4-oxobutanoyl)oxy)pyrrolidine-1-carboxylate (0.16 g, 188.9180 mol) in EA (1 mL) at 25° C. under open-air atmosphere. The reaction mixture was stirred for 2 h at 25° C., and then evaporated under reduced pressure. There was 4-[4-fluoro-5-[3-[4-fluoro-6-methoxy-2-[4-oxo-4-[(3S)-pyrrolidin-3-yl]oxy-butanoyl]benzothiophen-5-yl]oxypropoxy]-6-methoxy-isoindolin-2-yl]-4-oxo-butanoic acid (0.15 g, 130.3013 μmol, 68.9724% yield, 60% purity) obtained as a colorless oil, which was used directly in the next step. LCMS: (ESI, m/z): [M+H]⁺=691.210.

### Step c:

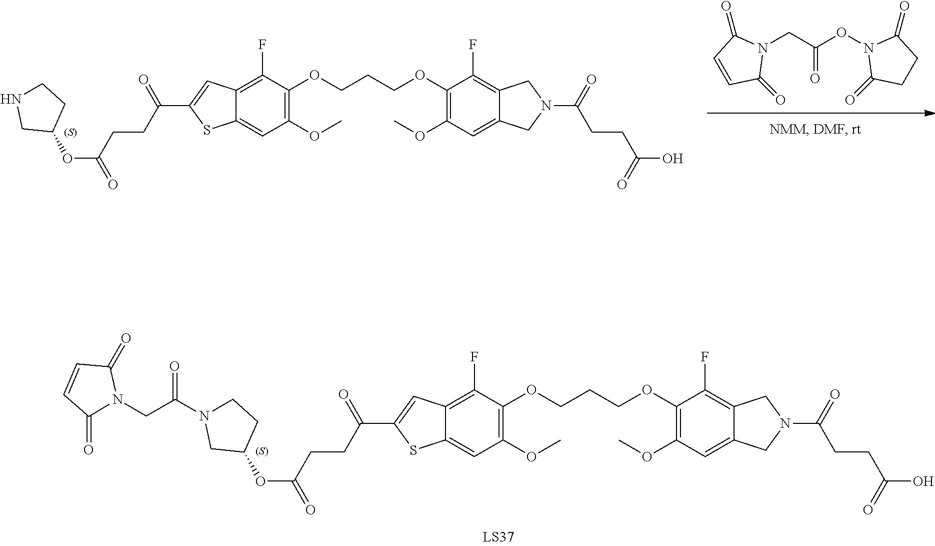

NMM (0.081 g, 800.8147 mol) was added to a solution of 4-[4-fluoro-5-[3-[4-fluoro-6-methoxy-2-[4-oxo-4-[(3S)-pyrrolidin-3-yl]oxy-butanoyl]benzothiophen-5-yl]oxypropoxy]-6-methoxy-isoindolin-2-yl]-4-oxo-butanoic acid (0.150 g, 217.1689 mol) and N-Succinimidyl maleimidoacetate (0.100 g, 396.5422 mol) in DMF (5 mL) at 25° C. under open-air atmosphere. The reaction mixture was stirred for 2 h at 25° C., and then purified on C18 column ACN/H₂O (0.1% TFA)(0-70%). The pure fraction was dried by lyophilization. The residue was purified on Prep-HPLC. The pure fraction was dried by lyophilization. There was 4-[5-[3-[2-[4-[(3S)-1-[2-(2,5-dioxopyrrol-1-yl)acetyl]pyrrolidin-3-yl]oxy-4-oxo-butanoyl]-4-fluoro-6-methoxy-benzothiophen-5-yl]oxypropoxy]-4-fluoro-6-methoxy-isoindolin-2-yl]-4-oxo-butanoic acid (0.0234 g, 28.2677 μmol, 13.0165% yield) obtained as a white solid. LCMS: (ESI, m/z): [M+H]⁺=828.220. ¹H NMR (600 MHz, DMSO-d₆) δ 12.14 (s, 1H), 8.32 (s, 1H), 7.56 (d, J=3.9 Hz, 1H), 7.12 (s, 2H), 6.90 (s, 0.5H), 6.87 (s, 0.5H), 5.36-5.32 (m, 0.5H), 5.27-5.22 (m, 0.5H), 4.78 (s, 2H), 4.57 (s, 1H), 4.55 (s, 1H), 4.37-4.30 (m, 1H), 4.28-4.24 (m, 2H), 4.23-4.17 (m, 3H), 3.89 (s, 3H), 3.84-3.79 (m, 0.5H), 3.78 (d, J=3.2 Hz, 3H), 3.76-3.73 (m, 0.5H), 3.66-3.61 (m, 0.5H), 3.58-3.50 (m, 2H), 3.40-3.35 (m, 2H), 3.30-3.25 (m, 0.5H), 2.70 (q, J=7.2 Hz, 2H), 2.61-2.55 (m, 2H), 2.49-2.45 (m, 2H), 2.24-2.16 (m, 0.5H), 2.09-2.02 (m, 3H), 1.97-1.90 (m, 0.5H).

### Example IV-38 Synthesis of Linker-STING Agonist Compound LS38

### Step a:

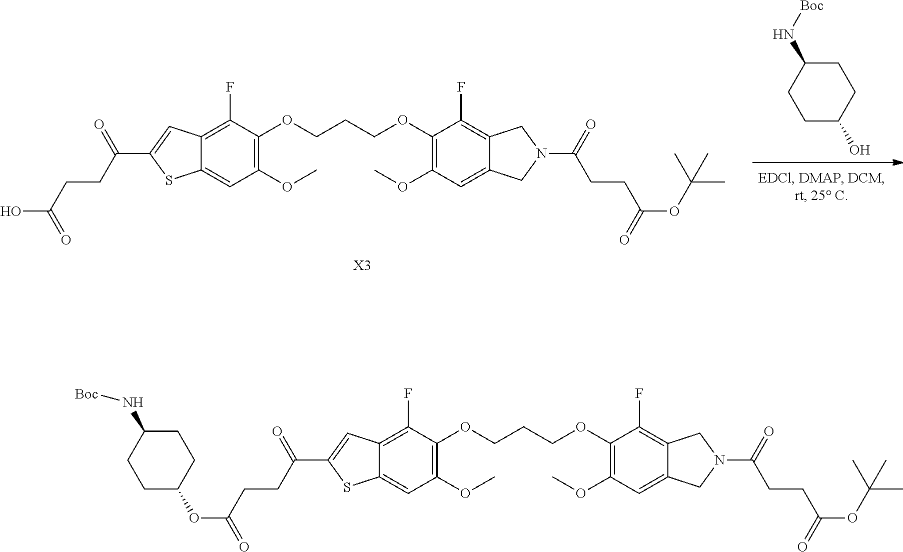 [SMILES: COc1cc2c(c(F)c1OCCCOc1c(OC)cc3sc(C(=O)CCC(=O)O)cc3c1F)CN(C(=O)CCC(=O)OC(C)(C)C)C2 ; CC(C)(C)OC(=O)N[C@H]1CC[C@H](O)CC1 ; "X3" ; COc1cc2c(c(F)c1OCCCOc1c(OC)cc3sc(C(=O)CCC(=O)O[C@H]4CC[C@H](NC(=O)OC(C)(C)C)CC4)cc3c1F)CN(C(=O)CCC(=O)OC(C)(C)C)C2]

Tert-butyl ((1r,4r)-4-hydroxycyclohexyl)carbamate (0.129 g, 599.1960 mol) was added to a solution of 4-(5-(3-((2-(4-(tert-butoxy)-4-oxobutanoyl)-4-fluoro-6-methoxyisoindolin-5-yl)oxy)propoxy)-4-fluoro-6-methoxybenzo[b]thiophen-2-yl)-4-oxobutanoic acid (0.209 g, 308.3925 mol), 3-(((ethylimino)methylene)amino)-N,N-dimethylpropan-1-amine hydrochloride(0.164 g, 855.4989 mol) and N-(4-pyridyl)dimethylamine, (0.089 g, 728.5110 mol) in dichloromethane (5 mL) at 25° C. The reaction mixture was stirred for 2 h at 40° C., and then evaporated under reduced pressure. The residue was purified on C18 column ACN/H₂O (0.1% TFA)(0-55%). The pure fraction was lyophilized overnight. There was [4-(tert-butoxycarbonylamino)cyclohexyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.186 g, 212.5760 mol, 68.9304% yield) obtained as a off white solid. LCMS: (ESI, m/z): [M+H]⁺=875.30. ¹H NMR (600 MHz, DMSO-d₆) δ 8.31 (d, J=2.3 Hz, 1H), 7.57 (d, J=3.9 Hz, 1H), 6.90 (s, 0.5H), 6.87 (s, 0.5H), 4.78 (s, 2H), 4.57 (s, 1H), 4.56-4.51 (m, 2H), 4.28-4.24 (m, 2H), 4.20 (t, J=6.0 Hz, 2H), 3.88 (s, 2H), 3.78 (d, J=1.7 Hz, 2H), 3.35 (t, J=6.5 Hz, 3H), 2.64 (t, J=6.4 Hz, 3H), 2.60-2.53 (m, 3H), 2.49-2.43 (m, 3H), 2.10-2.02 (m, 2H), 1.90-1.83 (m, 2H), 1.80-1.74 (m, 2H), 1.40 (s, 7H), 1.37 (s, 7H), 1.28-1.19 (m, 2H).

### Step b:

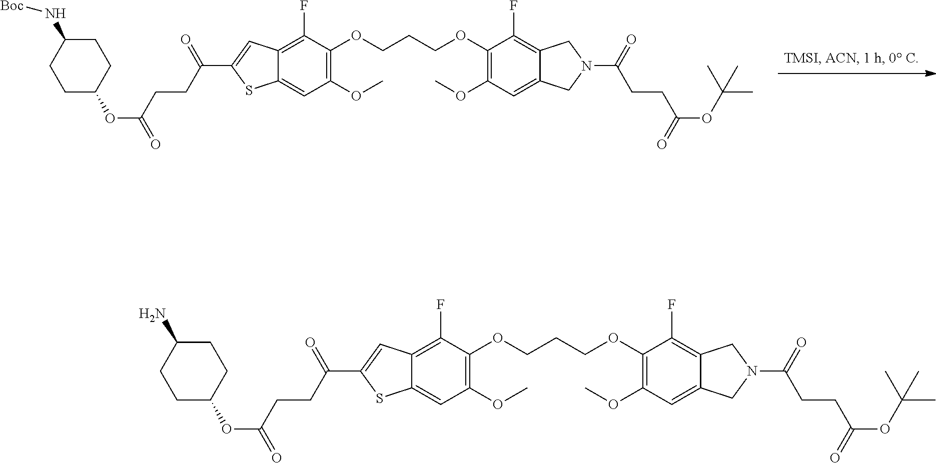 [SMILES: COc1cc2c(c(F)c1OCCCOc1c(OC)cc3sc(C(=O)CCC(=O)O[C@H]4CC[C@H](NC(=O)OC(C)(C)C)CC4)cc3c1F)CN(C(=O)CCC(=O)OC(C)(C)C)C2 ; COc1cc2c(c(F)c1OCCCOc1c(OC)cc3sc(C(=O)CCC(=O)O[C@H]4CC[C@H](N)CC4)cc3c1F)CN(C(=O)CCC(=O)OC(C)(C)C)C2]

N-(Trimethylsilyl)imidazole (0.095 g, 474.7792 mol) in MeCN (2 ml) was added to a solution of [4-(tert-butoxycarbonylamino)cyclohexyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.186 g, 212.5760 μmol) in MeCN (4 mL) at 0° C. The reaction mixture was stirred for lh at 0° C. The reaction mixture was quenched by addition of water (2 mL) at 25° C. The reaction mixture was purified on C18 column ACN/H₂O (0.1% TFA)(0-33%). The pure fraction was lyophilized overnight. There was (4-aminocyclohexyl)-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.129 g, 166.4805 mol, 78.3242% yield) obtained as a yellow solid.

LCMS: (ESI, m/z): [M+H]⁺=775.30.

### Step c:

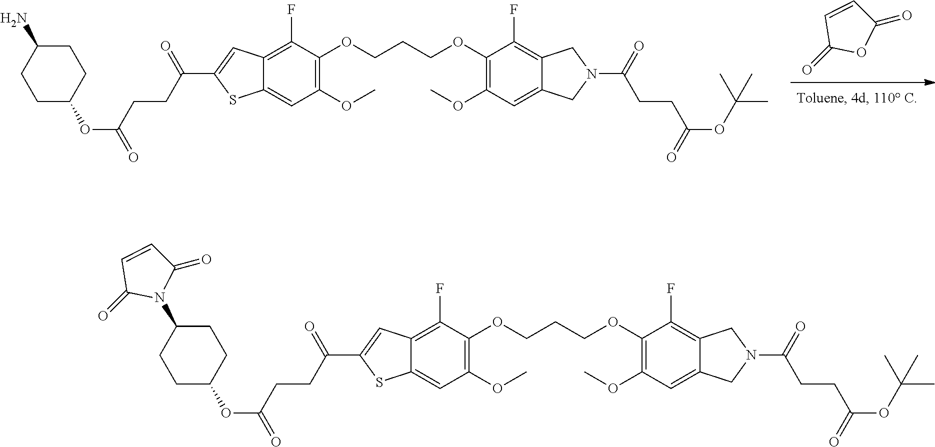 [SMILES: COc1cc2c(c(F)c1OCCCOc1c(OC)cc3sc(C(=O)CCC(=O)O[C@H]4CC[C@H](N)CC4)cc3c1F)CN(C(=O)CCC(=O)OC(C)(C)C)C2 ; O=C1C=CC(=O)O1 ; COc1cc2c(c(F)c1OCCCOc1c(OC)cc3sc(C(=O)CCC(=O)O[C@H]4CC[C@H](N5C(=O)C=CC5=O)CC4)cc3c1F)CN(C(=O)CCC(=O)OC(C)(C)C)C2]

(4-aminocyclohexyl)-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.129 g, 166.4805 mol) was added to a solution of maleic anhydride (0.086 g, 877.0427 mol) in toluene (4 mL) at 25° C. The reaction mixture was stirred for 4 d at 110° C., and then evaporated under reduced pressure. The residue was purified on C18 column ACN/0.1% TFA-H₂O (0-60%). The pure fraction solution was lyophilized overnight. There was [4-(2,5-dioxopyrrol-1-yl)cyclohexyl]4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.034 g, 39.7704 mol, 23.8889% yield) obtained as a brown solid. LCMS: (ESI, m/z): [M+H]⁺=855.30.

### Step d:

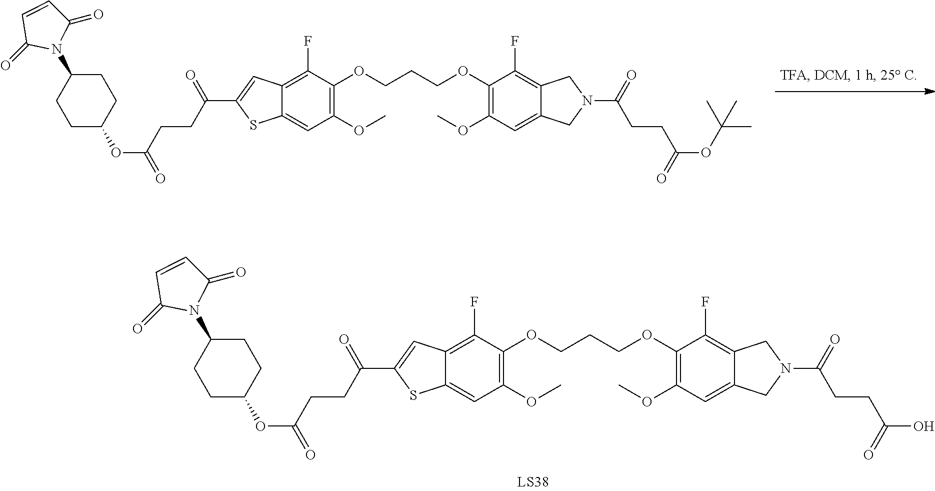 [SMILES: COc1cc2c(c(F)c1OCCCOc1c(OC)cc3sc(C(=O)CCC(=O)O[C@H]4CC[C@H](N5C(=O)C=CC5=O)CC4)cc3c1F)CN(C(=O)CCC(=O)OC(C)(C)C)C2 ; COc1cc2c(c(F)c1OCCCOc1c(OC)cc3sc(C(=O)CCC(=O)O[C@H]4CC[C@H](N5C(=O)C=CC5=O)CC4)cc3c1F)CN(C(=O)CCC(=O)O)C2]

[4-(2,5-dioxopyrrol-1-yl)cyclohexyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.033 g, 38.6007 μmol) was added to a solution of trifluoroacetic acid (0.2 mL) in dichloromethane (1 mL) at 25° C. The reaction mixture was stirred for 0.5h at 25° C., and then evaporated under reduced pressure. The residue was purified on C18 column ACN/H20 (0.1% TFA)(0-57%). The pure fraction was lyophilized overnight. There was 4-[5-[3-[2-[4-[4-(2,5-dioxopyrrol-1-yl)cyclohexoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (3.85 mg, 4.8197 mol, 12.4861% yield) obtained as a white solid. LCMS: (ESI, m/z): [M+H]⁺=799.25.

### Example IV-39 Synthesis of Linker-STING Agonist Compound LS39

### Step a:

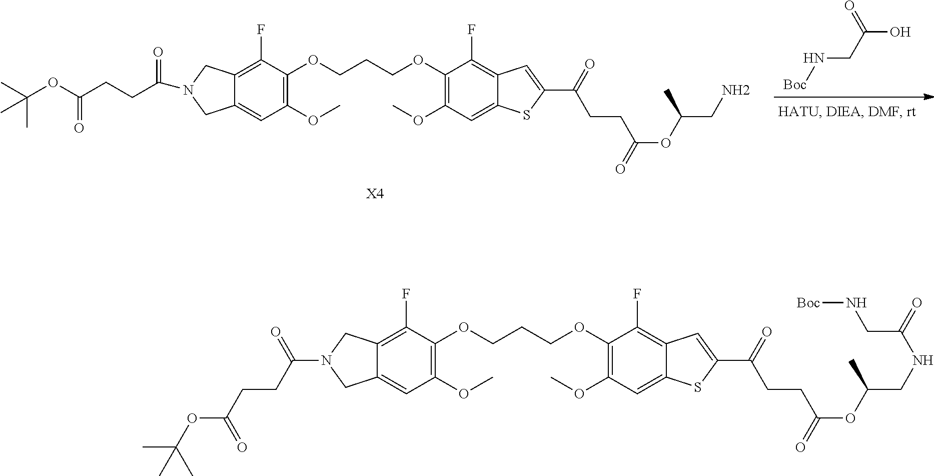 [SMILES: COc1cc2c(c(F)c1OCCCOc1c(OC)cc3sc(C(=O)CCC(=O)O[C@@H](C)CN)cc3c1F)CN(C(=O)CCC(=O)OC(C)(C)C)C2 ; CC(C)(C)OC(=O)NCC(=O)O ; "X4" ; COc1cc2c(c(F)c1OCCCOc1c(OC)cc3sc(C(=O)CCC(=O)O[C@@H](C)CNC(=O)CNC(=O)OC(C)(C)C)cc3c1F)CN(C(=O)CCC(=O)OC(C)(C)C)C2]

DIEA (0.078 mL, 447.8097 mol) was added to a solution of 4-(5-(3-((2-(4-(tert-butoxy)-4-oxobutanoyl)-4-fluoro-6-methoxyisoindolin-5-yl)oxy)propoxy)-4-fluoro-6-methoxybenzo[b]thiophen-2-yl)-4-oxobutanoic acid (X4, 0.107 g, 145.6175 mol), BOC-Glycine (0.031 g, 176.9588 mol), and HATU (0.085 g, 223.5493 μmol) in DMF (4 mL) at 25° C., wherein X4 was synthesized through the method as described in EXAMPLE IV-5. The reaction mixture was stirred for 2 h at 25° C. The resulting reaction mixture was directly purified on C18 column ACN/0.1% TFA-H₂O(0-60%). The pure fraction was concentranted under reduced pressure and then lyophilized overnight. There was [(1S)-2-[[2-(tert-butoxycarbonylamino)acetyl]amino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo- butanoate (0.098 g, 75.4507% yield) obtained as a white solid.. LCMS: (ESI, m/z): [M+H]⁺=892.350.

### Step b:

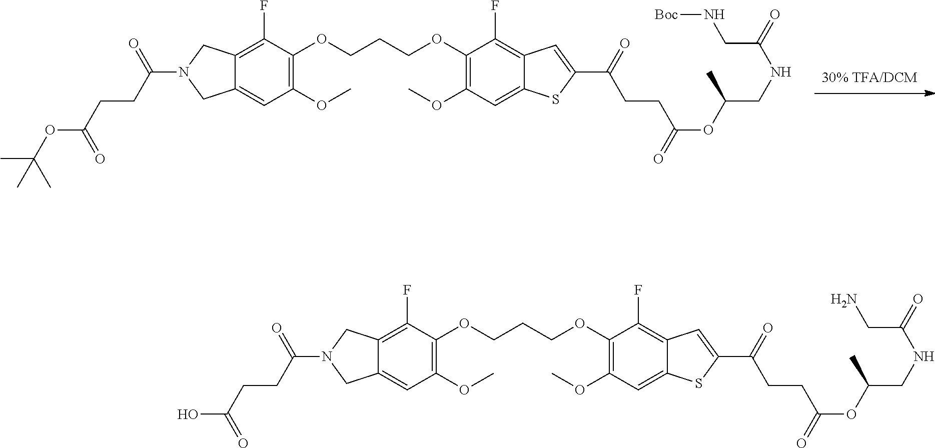 [SMILES: COc1cc2c(c(F)c1OCCCOc1c(OC)cc3sc(C(=O)CCC(=O)O[C@@H](C)CNC(=O)CNC(=O)OC(C)(C)C)cc3c1F)CN(C(=O)CCC(=O)OC(C)(C)C)C2 ; COc1cc2c(c(F)c1OCCCOc1c(OC)cc3sc(C(=O)CCC(=O)O[C@@H](C)CNC(=O)CN)cc3c1F)CN(C(=O)CCC(=O)O)C2]

(S)-1-(2-((tert-butoxycarbonyl)amino)acetamido)propan-2-yl-4-(5-(3-((2-(4-(tert-butoxy)-4-oxobutanoyl)-4-fluoro-6-methoxyisoindolin-5-yl)oxy)propoxy)-4-fluoro-6-methoxybenzo [b]thiophen-2-yl)-4-oxobutanoate (0.098 g, 109.8693 mol) was dissolved in 30% TFA/DCM (4 mL) at 0° C. The reaction mixture was stirred for 0.5 h at 25° C., and then concentrated under reduced pressure. The residue was lyophilized overnight. There was 4-[5-[3-[2-[4-[(1S)-2-[(2-aminoacetyl)amino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (0.076 g, 81.4021% yield) obtained as a brown solid. LCMS: (ESI, m/z): [M+H]⁺=736.300.

### Step c:

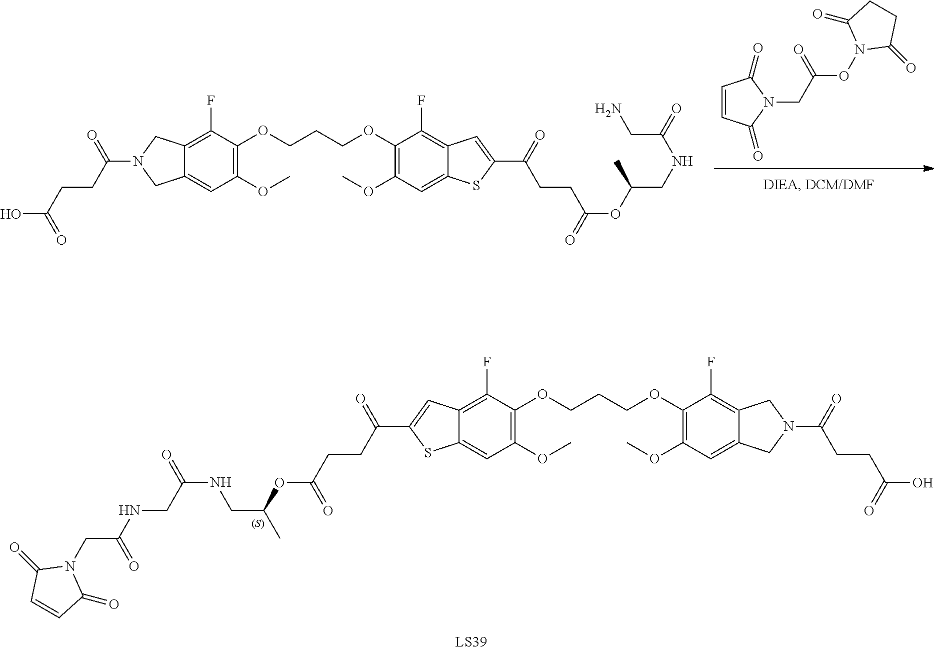

DIEA (0.025 mL, 143.5287 mol) and 4-[5-[3-[2-[4-[(1S)-2-[(2-aminoacetyl)amino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (0.076 g, 89.4360 mol) and N-Succinimidyl maleimidoacetate (0.034 g, 134.8243 mol) in DCM (3 mL) and DMF (1 mL) were added at 25° C. The reaction mixture was stirred for 3 h at 25° C., and then concentrated under reduced pressure. The residue was lyophilized overnight and further purified by prep-HPLC (the mobile phase: ACN/0.1% TFA-H₂O(0-60%) ). The pure fraction was lyophilized overnight. There was 4-[5-[3-[2-[4-[(1S)-2-[[2-[[2-(2,5-dioxopyrrol-1-yl)acetyl]amino]acetyl]amino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid (41.83 mg, 53.5847% yield) obtained as an off-white solid. LCMS: (ESI, m/z): [M+H]⁺=873.250.

¹H NMR (600 MHz, DMSO-d₆) δ 12.14 (s, 1H), 8.44 (t, J=5.7 Hz, 1H), 8.32 (s, 1H), 7.96 (t, J=5.9 Hz, 1H), 7.57 (d, J=3.7 Hz, 1H), 7.08 (s, 2H), 6.90 (s, 0.5H), 6.87 (s, 0.5H), 4.87-4.80 (m, 1H), 4.79 (s, 2H), 4.57 (s, 1H), 4.56 (s, 1H), 4.26 (t, J=5.9 Hz, 2H), 4.20 (t, J=6.0 Hz, 2H), 4.11 (s, 2H), 3.89 (s, 3H), 3.78 (d, J=3.4 Hz, 3H), 3.74-3.67 (m, 2H), 3.39-3.32 (m, 4H), 3.28-3.16 (m, 2H), 2.69-2.63 (m, 2H), 2.61-2.54 (m, 2H), 2.10-2.01 (m, 2H), 1.12 (d, J=6.4 Hz, 3H).

### Example IV-40 Synthesis of Linker-STING Agonist Compound LS40

### Step a:

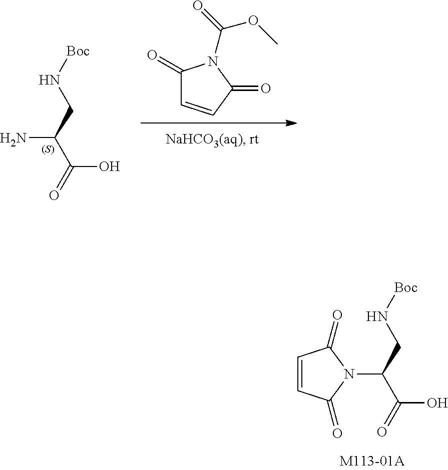

(2S)-2-amino-3-[(tert-butoxycarbonyl)amino]propanoic acid (2 g, 9.793 mmol, 1 equiv) and saturated sodium bicarbonate (40 mL) were added at 0° C. To the above mixture was added methyl 2,5-dioxopyrrolidine-1-carboxylate (4.62 g, 29.379 mmol, 3 equiv) in portions over 5 min at 0° C. The resulting mixture was stirred for 16 h at room temperature. Desired product could be detected by LCMS. The residue was acidified to a pH of 5 with 1 N KHSO₄. The resulting mixture was extracted with EtOAc (3×30 mL). The organic phase was collected and dried by anhydrous Na₂SO₄. After filtration, the filtrate was concentrated under reduced pressure. The residue was purified by reversed-phase flash chromatography with the following conditions: column, C18 gel; mobile phase, MeCN in Water, 0% to 60% gradient in 15 min; detector, UV 220 nm. This resulted in (2S)-3-[(tert-butoxycarbonyl)amino]-2-(2,5-dioxopyrrol -1-yl)propanoic acid (809 mg, 28.77%) as a white solid.

LCMS: (ESI, m/z): [M−H]—=283.050. 1H NMR (300 MHz, DMSO-d6) δ 7.09 (s, 2H), 6.97 (t, J=6.3 Hz, 1H), 4.59 (m, 1H), 3.61-3.55 (m, 1H), 3.56-3.51 (m, 1H), 1.32 (s, 9H).

### Step b:

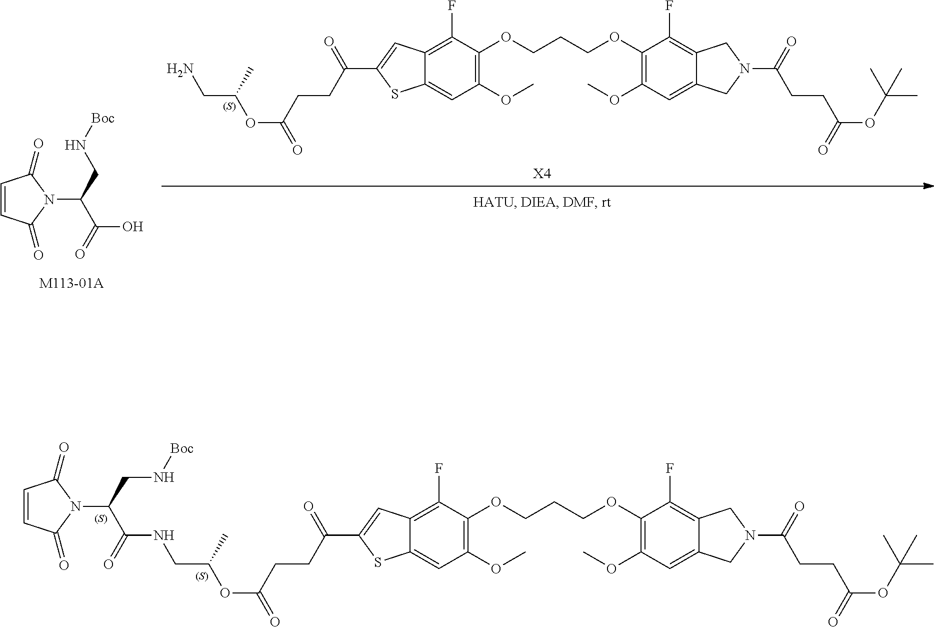

(S)-1-aminopropan-2-yl-4-(5-(3-((2-(4-(tert-butoxy)-4-oxobutanoyl)-4-fluoro-6-methoxyisoindolin-5-yl)oxy) propoxy)-4-fluoro-6-methoxybenzo[b]thiophen-2-yl)-4-oxobutanoate (0.05 g, 68.0455 mol) was added to a solution of (2S)-3-[(tert-butoxycarbonyl)amino]-2-(2,5-dioxopyrrol-1-yl)propanoic acid (0.046 g, 161.8211 mol), HATU (0.061 g, 160.4295 mol) and N,N-Diisopropylethylamine (0.031 g, 239.8594 mol) in DMF (3 mL) at 25° C. under open-air atmosphere. The reaction mixture was stirred for 2 h at 25° C., and then purified on C18 column ACN/H₂O (0.1% TFA)(0-60%). The pure fraction was concentrated and dried under vacuo. There was [(1S)-2-[[(2S)-3-(tert-butoxycarbonylamino)-2-(2,5-dioxopyrrol-1-yl)propanoyl]amino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy- benzothiophen-2-yl]-4-oxo-butanoate (0.06 g, 59.9370 mol, 88.0836% yield) obtained as a yellow solid. LCMS: (ESI, m/z): [M+H]⁺=1001.060.

### Step c:

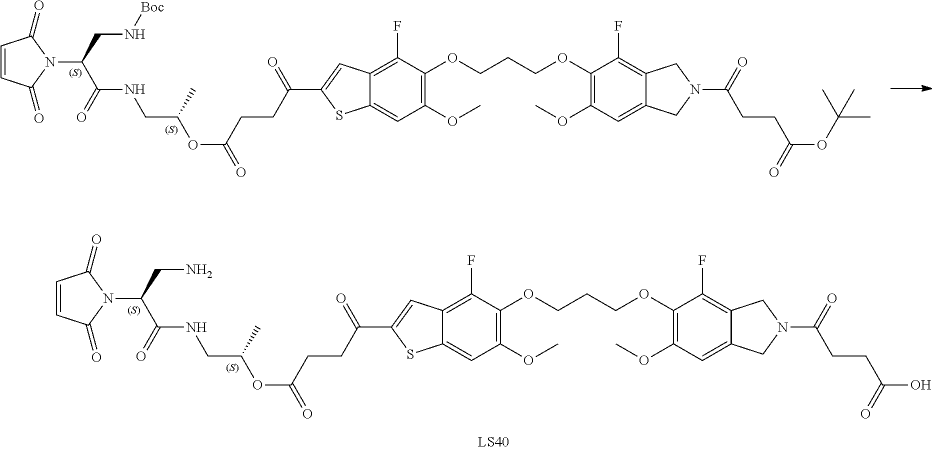

Trifluoroacetic acid (2 mL) was added to a solution of [(1S)-2-[[(2S)-3-(tert-butoxycarbonylamino)-2-(2,5-dioxopyrrol-1-yl)propanoyl]amino]-1-methyl-ethyl]-4-[5-[3-[2-(4-tert-butoxy-4-oxo-butanoyl)-4-fluoro-6-methoxy-isoindolin-5-yl]oxypropoxy]-4-fluoro-6-methoxy-benzothiophen-2-yl]-4-oxo-butanoate (0.056 g, 55.9413 mol) in DCM (3 mL) at 25° C. under open-air atmosphere. The reaction mixture was stirred for 2 h at 25° C. The reaction mixture was evaporated under reduced pressure. The residue was purified on Prep-HPLC. The pure fraction was concentrated and dried by lyophilization. There was 4-[5-[3-[2-[4-[(1S)-2-[[(2S)-3-amino-2-(2,5-dioxopyrrol-1-yl)propanoyl]amino]-1-methyl-ethoxy]-4-oxo-butanoyl]-4-fluoro-6-methoxy-benzothiophen-5-yl]oxypropoxy]-4-fluoro-6-methoxy-isoindolin-2-yl]-4-oxo-butanoic acid obtained as a white solid. LCMS: (ESI, m/z): [M+H]⁺=845.240. ¹H NMR (600 MHz, DMSO-d₆) δ 12.06 (s, 1H), 8.41 (t, J=6.0 Hz, 1H), 8.31 (d, J=2.3 Hz, 1H), 7.87 (s, 3H), 7.57 (d, J=4.6 Hz, 1H), 7.15 (s, 2H), 6.90 (s, 0.5H), 6.88 (s, 0.5H), 4.83-4.74 (m, 4H), 4.57 (s, 1H), 4.55 (s, 1H), 4.28-4.23 (m, 2H), 4.22-4.17 (m, 2H), 3.89 (d, J=2.0 Hz, 3H), 3.78 (d, J=6.5 Hz, 3H), 3.51-3.46 (m, 1H), 3.31-3.28 (m, 3H), 3.25-3.19 (m, 1H), 3.11-3.04 (m, 1H), 2.72-2.54 (m, 4H), 2.50-2.46 (m, 2H), 2.08-2.02 (m, 2H), 1.08 (d, J=6.3 Hz, 3H).

### Example IV-41 Synthesis of Linker-STING Agonist Compound LS41

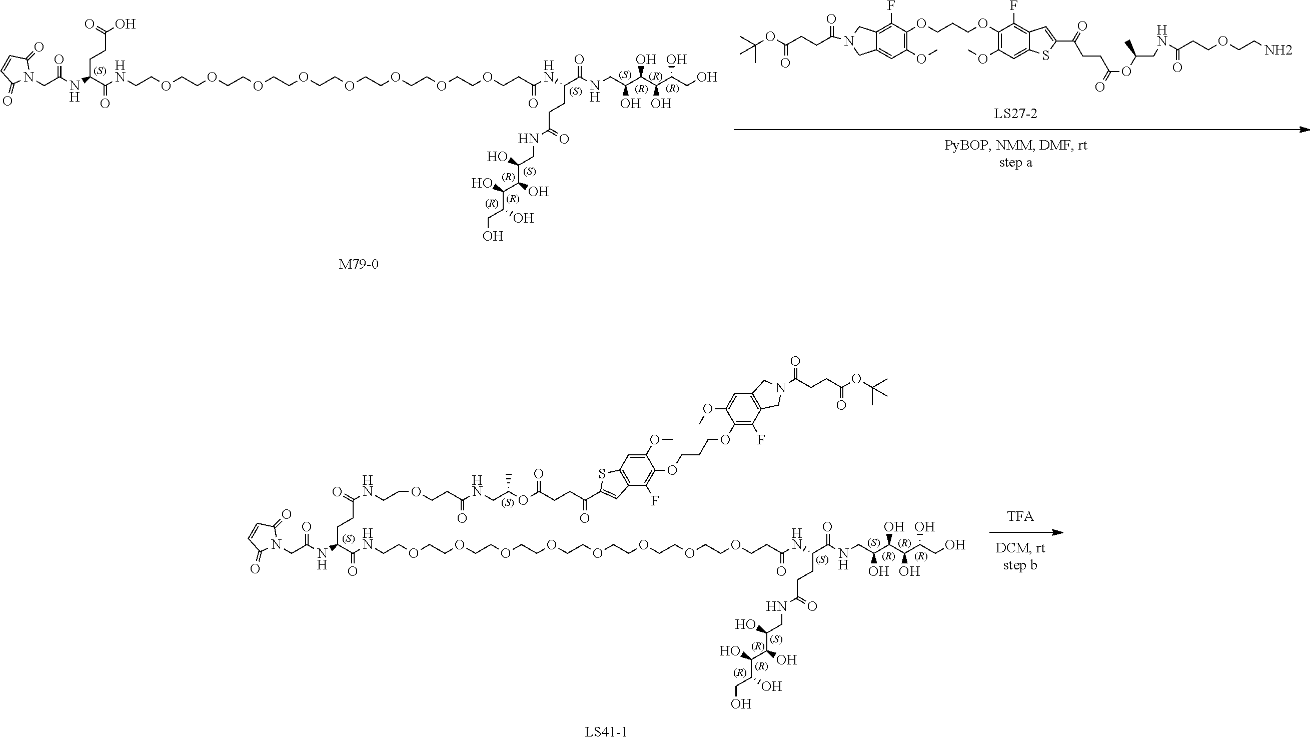

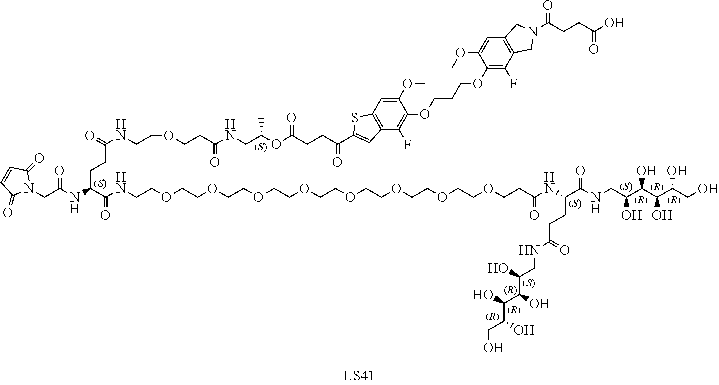

LS27-2 (0.093 g, 109.4205 mol) and NMM (0.034 g, 336.1444 mol) was added to a solution of M79-0 (0.155 g, 133.2560 mol) and PyBOP (0.162 g, 311.3041 mol) in DMF (3 mL) at 20° C. under N₂ atmosphere. The reaction mixture was stirred for 2 h at 20° C. The mixture was purified on C18 column ACN/H₂O (0.1% TFA)(0-70%). The pure fractions was concentrated and then lyophilized overnight. There was LS41-1 (0.230 g, 94.8% yield) obtained as a white solid. LCMS: (ESI, m/z): [M+2H]²⁺=998.350.

### Step b:

LS41-1 (0.218 g, 109.2683 mol) was added to a solution of TFA (0.5 mL) and DCM (5 mL) at 20° C. under open-air atmosphere. The reaction mixture was stirred for 0.5 h at 20° C. The reaction mixture was evaporated under reduced pressure. The residue was purified on Prep-HPLC. The pure fractions was lyophilized overnight. There was LS41 (70.0 mg, 33.0% yield) obtained as a white solid. LCMS: (ESI, m/z): [M+2H]²⁺=970.250.

### Example IV-42 Synthesis of Linker-STING Agonist Compound LS42

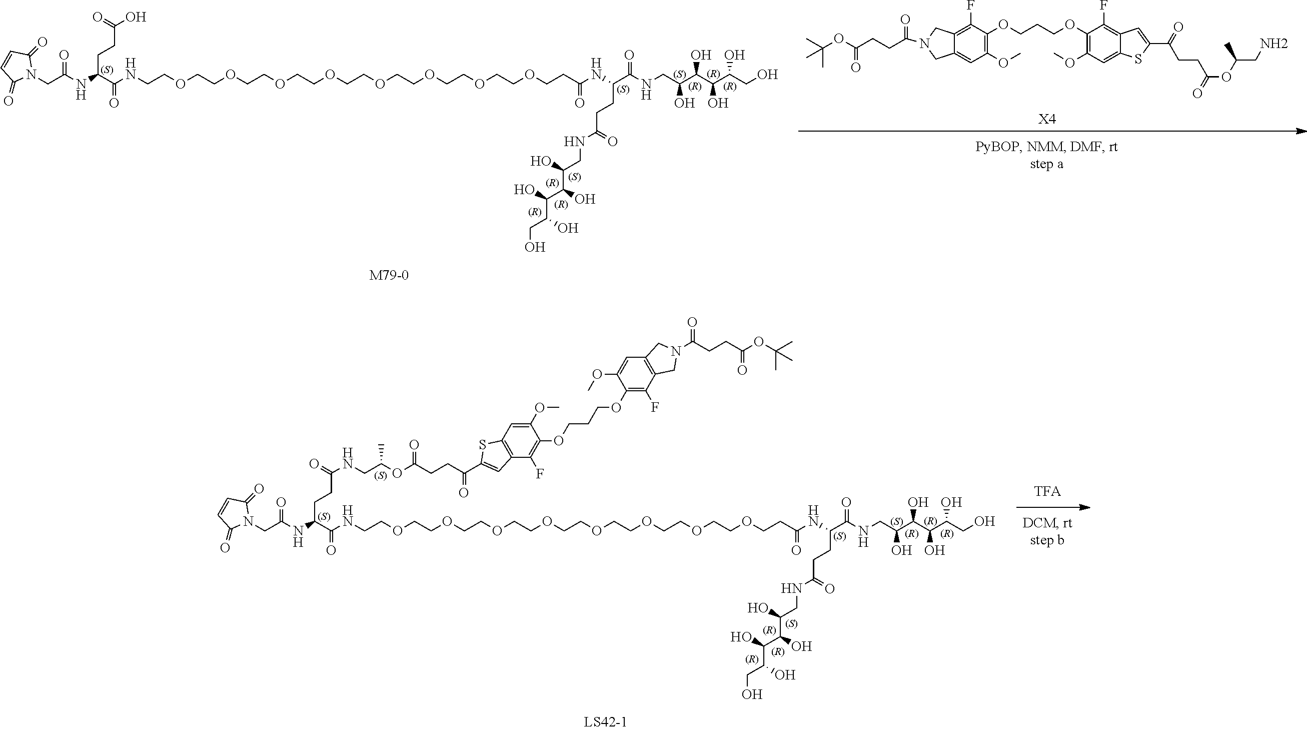

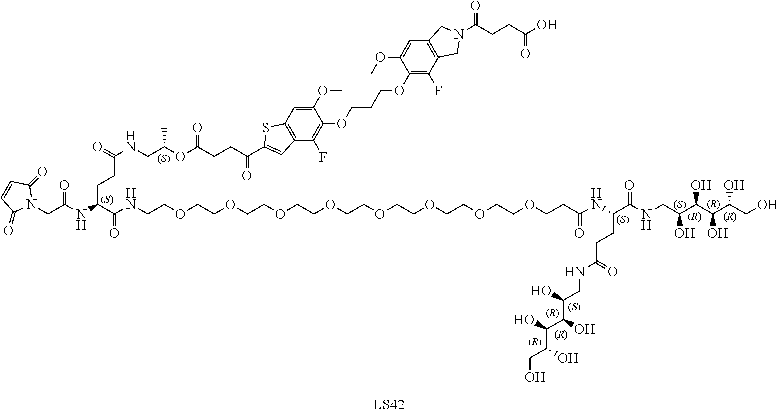

### Step a:

X4 (0.059 g, 80.2937 mol) and NMM (0.046 mL, 418.4009 mol) was added to a solution of M79-0 (0.103 g, 88.5511 mol) and PyBOP (0.137 g, 263.2634 mol) in DMF (6 mL) at 20° C. under N₂ atmosphere. The reaction mixture was stirred for 2 h at 20° C. The mixture was purified on C18 column ACN/H₂O (0.1% TFA)(0-45%). The pure fractions was concentrated and then lyophilized overnight. There was LS42-1 (0.142 g, 85.3% yield) obtained as a yellowish solid. LCMS: (ESI, m/z): [M+2H]²⁺=940.800.0.

### Step b:

LS42-1 (0.141 g, 75.0016 mol) was dissolved in 20% TFA/DCM (6 mL) at 20° C. under open-air atmosphere. The reaction mixture was stirred for 0.5 h at 20° C. The reaction mixture was evaporated under reduced pressure. The residue was purified on Prep-HPLC. The pure fractions was lyophilized overnight. There was LS42 (0.0381 g, 20.8898 mol, 27.9% yield) obtained as a white solid. LCMS: (ESI, m/z): [M+2H]²⁺=912.750.

### Example V Preparation of iADCs

Trastuzumab; or the anti-CD73 antibody AIgG1 (with the amino acid sequence of the heavy chain as shown in SEQ ID NO: 1 and the amino acid sequence of the light chain sequence as shown in SEQ ID NO: 2); or anti-c-Met antibody (with the amino acid sequence of the heavy chain as shown in SEQ ID NO: 8 and the amino acid sequence of the light chain sequence as shown in SEQ ID NO: 9), was centrifuged by using an ultrafiltration tube with a molecular weight cut-off of 30KDa and transferred into the conjugation buffer of 50 mM PBS (pH 6.0-7.0), adjusted to a concentration of 5-8 mg/mL. TCEP (tris(2-chloroethyl) phosphate, 10 mM) solution which is 8-12 times the molar number of the antibody was added, mixed well, placed in a constant temperature shaker at 37° C., and reacted in a water bath for 2-2.5h. After completion of the reduction reaction, the temperature of the reaction system was lowered to 25° C. After reduction, the antibody can be directly used for subsequent conjugation. 10 mM linker-STING agonist was prepared and dissolved in DMA (N,N-dimethylacetamide) slowly according to the molar ratio of linker-STING agonist to antibody to be 8:1-16:1, and mixed well, and then reacted in a water bath at 25° C. for 1-1.5h. After the reaction, the reaction mixture was centrifuged by using an ultrafiltration tube with a molecular weight cut-off of 30KDa, and transferred to the storage buffer (20 mM succinate, 8% sucrose, pH 5.5). The unconjugated linker-STING agonist and free small molecules such as DMA were removed. Purity was detected by size exclusion high performance liquid chromatography (SEC-HPLC) and conjugation was measured by hydrophobic high performance liquid chromatography (HIC-HPLC) (Table 5, 6a, 7). As shown in Table 5, LSO represents the following compound, the preparation method for which was described in WO2021202984A1, the corresponding payload was named Compound CO in the present disclosure:

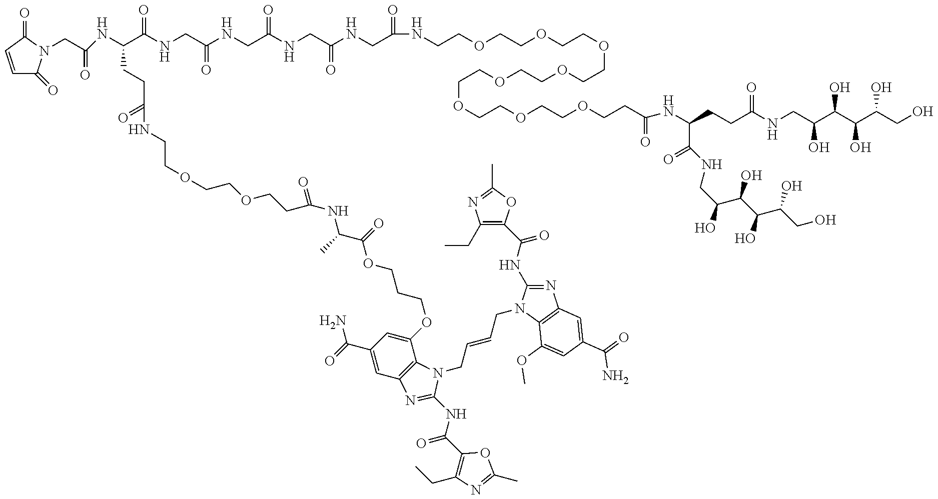 [SMILES: CCc1nc(C)oc1C(=O)Nc1nc2cc(C(N)=O)cc(OC)c2n1C/C=C/Cn1c(NC(=O)c2oc(C)nc2CC)nc2cc(C(N)=O)cc(OCCCOC(=O)[C@H](C)NC(=O)CCOCCOCCNC(=O)CC[C@H](NC(=O)CN3C(=O)C=CC3=O)C(=O)NCC(=O)NCC(=O)NCC(=O)NCC(=O)NCCOCCOCCOCCOCCOCCOCCOCCOCCC(=O)N[C@@H](CCC(=O)NC[C@H](O)[C@@H](O)[C@H](O)[C@H](O)CO)C(=O)NC[C@H](O)[C@@H](O)[C@H](O)[C@H](O)CO)c21]

50 mM PBS (pH 6.0-7.0), adjusted to a concentration of 5-8 mg/mL. TCEP (tris(2-chloroethyl) phosphate, 20 mM) solution which is 6-24 times the molar number of the antibody was added, mixed well, placed in a constant temperature shaker at 37° C., and reacted in a water bath for 2-2.5h. After completion of the reduction reaction, the temperature of the reaction system was lowered to 25° C. After reduction, the antibody can be directly used for subsequent conjugation. 25 mM linker-STING agonist was prepared and dissolved in DMA (N,N-dimethylacetamide) slowly according to the molar ratio of linker-STING agonist to antibody to be 36:1-42:1, and mixed well, and then reacted in a water bath at 25° C. for 1-1.5h. After the reaction, the reaction mixture was centrifuged by using an ultrafiltration tube with a molecular weight cut-off of 30KDa, and transferred to the storage buffer (20 mM succinate, 8% sucrose, pH 5.5). The unconjugated linker-STING agonist and free small molecules such as DMA were removed. Purity was detected by size exclusion high performance liquid chromatography (SEC-HPLC) and conjugation was measured by hydrophobic high performance liquid chromatography (HIC-HPLC) (Tables 6).

Amino acid sequence of the anti-CD73 antibody AIgG1 used in the current example:

The heavy chain:
(SEQ ID NO: 1)
QVQLVQSGAEVKKPGASVKVSCKASGYTFTSYWMHWVRQAPGQGLEWMG
YINPSSGYTKSNQKFKDRVTMTADTSTSTAYMELSSLRSEDTAVYYCGR
WLLSAWFAYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK
The light chain:
(SEQ ID NO: 2)
DIQMTQSPSSLSASVGDRVTITCKASQDINTYLSWFQQKPGKSPKSLIY
RSNILVDGVPSRFSGSGSGQDYTLTISSLQPEDFAIYYCLQYDDFPYTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Amino acid sequence of the anti-CD73 antibody AIgG2 used in the current example:

The heavy chain:
(SEQ ID NO: 2)
QVQLVQSGAEVKKPGASVKVSCKASGYTFTSYWMHWVRQAPGQGLEWMG
YINPSSGYTKSNQKFKDRVTMTADTSTSTAYMELSSLRSEDTAVYYCGR
WLLSAWFAYWGQGTLVTVSSASTKGPSVFPLAPCSRSTSESTAALGCLV
KDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPK
DTLMISRTPEVTCVVVDVSHEDPEVQFNWYVDGVEVHNAKTKPREEQFN
STFRVVSVLTVVHQDWLNGKEYKCKVSNKGLPAPIEKTISKTKGQPREP
QVYTLPPSREEMTKNQVSLTCLVKGFYPSDIAVEWESNGQPENNYKTTP
PMLDSDGSFFLYSKLTVDKSRWQQGNVFSCSVMHEALHNHYTQKSLSLS
PGK
The light chain:
(SEQ ID NO: 3)
DIQMTQSPSSLSASVGDRVTITCKASQDINTYLSWFQQKPGKSPKSLIY
RSNILVDGVPSRFSGSGSGQDYTLTISSLQPEDFAIYYCLQYDDFPYTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Amino acid sequence of the anti- c-Met antibody used in the current example:

The heavy chain:
(SEQ ID NO: 8)
QVQLVQSGAEVKKPGASVKVSCKASGYIFTAYTMHWVRQAPGQGLEWMG
WIKPNNGLANYAQKFQGRVTMTRDTSISTAYMELSRLRSDDTAVYYCAR
SEITTEFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK
The light chain:
(SEQ ID NO: 9)
DIVMTQSPDSLAVSLGERATINCKSSESVDSYANSFLHWYQQKPGQPPK
LLIYRASTRESGVPDRFSGSGSGTDFTLTISSLQAEDVAVYYCQQSKED
PLTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

TABLE 5
Conjugation rate and SEC purity of conjuagtes of Trastuzumab with
different STING agonists

| | Corresponding | Total HIC | |
|---|---|---|---|
| Serial No. of | linker-STING | Conjugation rate | SEC purity |
| Conjugates | agonist compounds | (%) | (%) |
| iADC-a0 | LS0 | 96.2 | 99.27 |
| iADC-a1 | LS1 | 99.3 | 95.20 |
| iADC-a2 | LS2 | 98.6 | 89.54 |
| iADC-a3 | LS3 | 98.9 | 97.75 |
| iADC-a4 | LS4 | 100.0 | 96.91 |
| iADC-a5 | LS5 | 99.7 | 99.29 |
| iADC-a6 | LS6 | 100 | 99.44 |
| iADC-a7 | LS7 | 100 | 99.45 |
| iADC-a8 | LS8 | 98.3 | 97.00 |
| iADC-a9 | LS9 | 98.2 | 97.52 |
| iADC-a10 | LS10 | 99.9 | 97.15 |
| iADC-a11 | LS11 | 99.9 | 96.53 |
| iADC-a12 | LS12 | 97.8 | 99.27 |
| iADC-a13 | LS13 | 100.0 | 97.55 |
| iADC-a14 | LS14 | 100.0 | 98.93 |
| iADC-a15 | LS15 | 100.0 | 99.52 |
| iADC-a16 | LS16 | 100.0 | 98.71 |
| iADC-a17 | LS17 | 100.0 | 98.47 |
| iADC-a18 | LS18 | 99.7 | 94.44 |
| iADC-a19 | LS19 | 100.0 | 98.96 |
| iADC-a20 | LS20 | 100.0 | 98.26 |
| iADC-a21 | LS21 | 100.0 | 99.23 |
| iADC-a22 | LS22 | 100.0 | 98.81 |
| iADC-a23 | LS23 | 100.0 | 92.39 |
| iADC-a24 | LS24 | 100.0 | 98.67 |
| iADC-a25 | LS25 | 100.0 | 97.99 |
| iADC-a26 | LS26 | 100.0 | 99.34 |
| iADC-a27 | LS27 | 100.0 | 98.73 |
| iADC-a28 | LS28 | 100.0 | 98.32 |
| iADC-a29 | LS29 | 100.0 | 98.24 |
| iADC-a30 | LS30 | 100.0 | 98.32 |
| iADC-a31 | LS31 | 99.5 | 98.41 |
| iADC-a32 | LS32 | 100.0 | 97.29 |
| iADC-a33 | LS33 | 100.0 | 97.98 |
| iADC-a34 | LS34 | 100.0 | 98.27 |
| iADC-a35 | LS35 | 100.0 | 97.60 |
| iADC-a36 | LS36 | 100.0 | 97.62 |
| iADC-a37 | LS37 | 100.0 | 97.10 |
| iADC-a38 | LS38 | 100.0 | 96.69 |
| iADC-a39 | LS39 | 100.0 | 97.58 |
| iADC-a40 | LS40 | 100.0 | 95.34 |
| iADC-a41 | LS41 | 100 | 94.63 |
| iADC-a42 | LS42 | 100 | 98.06 |

TABLE 6
Conjugation rate and SEC purity of conjuagtes of anti-CD73 antibody
AIgG2 with different STING agonists

| | Corresponding | Total HIC | |
|---|---|---|---|
| Serial No. of | linker-STING | Conjugation rate | SEC purity |
| Conjugates | agonist compounds | (%) | (%) |
| iADC-2b0 | LS0 | 97.3 | 97.64 |
| iADC-2b1 | LS1 | 98.3 | 100.00 |
| iADC-2b5 | LS5 | 99.3 | 99.11 |
| iADC-2b21 | LS21 | 99.1 | 98.59 |
| iADC-2b22 | LS22 | 99.7 | 98.50 |
| iADC-2b27 | LS27 | 98.7 | 99.17 |
| iADC-2b31 | LS31 | 97.1 | 98.86 |

TABLE 6a
Conjugation rate and SEC purity of conjuagtes of anti-CD73 antibody
AIgG1 with different STING agonists

| | Corresponding | Total HIC | |
|---|---|---|---|
| Serial No. of | linker-STING | Conjugation rate | SEC purity |
| Conjugates | agonist compounds | (%) | (%) |
| iADC-1b5 | LS5 | 100 | 95.30 |
| iADC-1b21 | LS21 | 100 | 97.01 |

TABLE 7
Conjugation rate and SEC purity of conjuagtes of anti-c-Met antibody
with different STING agonists

| | Corresponding | Total HIC | |
|---|---|---|---|
| Serial No. of | linker-STING | Conjugation rate | SEC purity |
| Conjugates | agonist compounds | (%) | (%) |
| iADC-c1 | LS31 | 98.4 | 97.6 |
| iADC-c2 | LS21 | 99.5 | 96.95 |

### Example VI-1 Cancer Cell Killing Activity Assay

Activity of SK—OV-3, MDA-MB-231, Calu-1, EBC-1 tumor cell targeted antibody drug conjugates in cancer cell/PBMC co-cultures. Cell proliferation analysis was conducted by CellTiter-Glo (Promega, Cat #G1111). SK—OV-3, MDA-MB-231, Calu-1, EBC-1 tumor cells were be harvested during the logarithmic growth period and counted with hemocytometer. The cell viability was over 90% by trypan blue exclusion. Cancer cells were adjusted to a concentration of 5.0×10⁴ cells/mL with complete medium (RPMI-1640 medium supplemented with 10% (v/v) fetal bovine serum). 50 μL cell suspensions were added to 96-well plates to a final cell density of 2.5×10³ cells/well. The plates were cultured in the temperature of 37° C., 5% CO₂ and 95% humidity overnight. A range of compound dilutions (0.00 μM to 1000 nM based on payload, 5-fold serial dilutions in growth medium) of the 50 μL test articles were added to each well and the plate was incubated for 20 mins at 37° C. PBMCs (1.5×10⁵ cells/50 μL) were then added to each well and the plates were then cultured in the temperature of 37° C., 5% CO₂ and 95% humidity for 72 hours. After incubation, the supernatant was removed. CellTiter-Glo reagent was added into the 96-well plates, followed by a shaking incubation for 2 mins and a static incubation for 10 mins at room temperature. The Luminosity values were recorded using a microplate spectrophotometer (Spark, Tecan), and the data was processing using GraphPad 8.0 to obtain IC₅₀ values (Tables 8-11).

TABLE 8
The cancer cell killing activity [IC₅₀ values (nM)] of the
Trastuzumab-iADCs and their corresponding free payloads
in SK-OV-3/PBMC co-cultures

| | | IC₅₀ values (nM) for the |
|---|---|---|
| iADCs | IC₅₀ values (nM) for iADCs | corresponding free payload |
| iADC-a0 | 0.07 | 51.9 |
| iADC-a5 | 0.10 | 69.2 |
| iADC-a21 | 0.02 | 69.2 |
| iADC-a22 | 0.06 | 69.2 |
| iADC-a24 | 0.12 | 69.2 |
| iADC-a25 | 0.09 | 69.2 |
| iADC-a27 | 0.13 | 69.2 |
| iADC-a28 | 0.03 | 43.7 |
| iADC-a31 | 0.10 | 69.2 |

TABLE 9
The cancer cell killing activity [IC₅₀ values (nM)] of the
Trastuzumab-iADCs and their corresponding free payloads
in SK-OV-3/PBMC co-cultures

| | | IC₅₀ values (nM) for the |
|---|---|---|
| iADCs | IC₅₀ values (nM) for iADCs | corresponding free payload |
| iADC-a0 | 0.03 | 21.2 |
| iADC-a21 | 0.07 | 21.2 |
| iADC-a32 | 0.06 | 21.2 |
| iADC-a34 | 0.02 | 21.2 |
| iADC-a35 | 0.02 | 21.2 |
| iADC-a36 | 0.06 | 21.2 |
| iADC-a37 | 0.10 | 21.2 |
| iADC-a38 | 0.04 | 21.2 |
| iADC-a39 | 0.09 | 21.2 |
| iADC-a41 | 0.19 | 674 |
| iADC-a42 | 0.18 | 674 |

TABLE 10
The cancer cell killing activity [IC₅₀ values (nM)] of the anti-CD73
antibody-iADCs and their corresponding free payloads in
MDA-MB-231/PBMC co-culture and Calu-1/PBMC co-culture

| | MDA-MB-231/PBMC co-culture | | Calu-1/PBMC co-culture | |
|---|---|---|---|---|
| | | IC₅₀ values | | IC₅₀ values |
| | IC₅₀ values | (nM) for the | IC₅₀ values | (nM) for the |
| | (nM) for | corresponding | (nM) for | corresponding |
| iADCs | iADCs | free payload | iADCs | free payload |
| iADC- | 7.70 | 45.2 | 16.4 | 150 |
| 1b5 | | | | |
| iADC- | 4.13 | 45.2 | 5.24 | 150 |
| 1b21 | | | | |
| iADC- | 9.77 | 45.2 | 10.9 | 150 |
| 2b5 | | | | |
| iADC- | 6.50 | 45.2 | 1.74 | 150 |
| 2b21 | | | | |

TABLE 11
The cancer cell killing activity [IC₅₀ values (nM)] of the c-Met
antibody-iADC and their corresponding free payloads in
EBC-1/PBMC co-cultures

| | EBC-1/PBMC co-culture | |
|---|---|---|
| | | IC₅₀ values (nM) for the |
| iADCs | IC₅₀ values (nM) for iADCs | corresponding free payload |
| iADC-c1 | 0.052 | 187 |
| iADC-c2 | 0.086 | 187 |

### Example VI-2 the CXCL10 Induction Assay

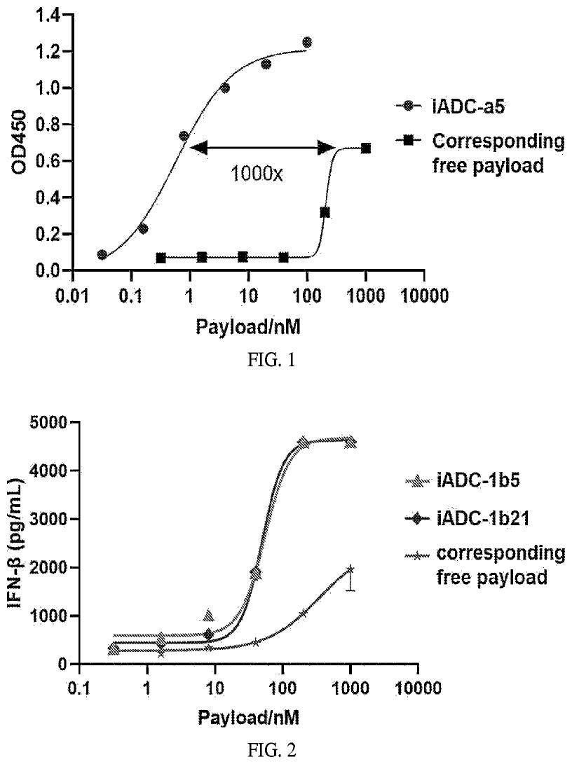

The induction of CXCL1O was evaluated by SK—OV-3 human ovarian adenocarcinoma cells/PBMCs co-culture assay. SK—OV-3 cells were seeded in 96 well plate (10,000 cells/well) and allowed to attach for overnight in RPMI-1640 medium with 10% FBS. A range of compound dilutions (0.0mnM to 1000 nM based on payload, 5-fold serial dilutions in growth medium) of the test articles were added to each well and the plate was incubated for 20 min at 37° C. PBMCs (1.5×10⁵ cells) were then added to each well and the plates will be cultured in the temperature of 37° C., 5% CO₂ and 95% humidity for 24 hours. Particulates were removed by centrifugation and samples were stored at −80° C. FIG. 1 shows the quantitative determination of human CXCL10 in cell culture supernates by ELISA kit (#DIP100, R&D).

### Example VI-3 IFN-β Induction

The induction of IFN-β was evaluated by MAD-MB-231 human breast adenocarcinoma cells/THP-1 co-culture assay. MAD-MB-231 cells were seeded in 96 well plate (10,000 cells/well) and incubated overnight in RPMI-1640 medium with 10% FBS. A range of compound dilutions (0.032 nM to 1000 nM based on payload, 5-fold serial dilutions in growth medium) of the test articles were added to each well and the plate was incubated for 20 mins at 37° C. THP-1 (5×10⁴ cells) were then added to each well and the plates will be cultured in the temperature of 37° C., 5% CO₂ and 95% humidity for 24 hours. Particulates were removed by centrifugation and samples were stored at −80° C. The quantitative determination of human IFN-β in cell culture supernates by HTRF kit (#62HIFNBPEG, Cisbio). FIG. 2 shows that the iADCs of the present disclosure resulted in significant IFN-β induction.

### Example VI-4 Plasma Stability Assay

Test articles were spiked into 1.5 mL of plasma at a concentration of 1 μM and incubated at 37° C. Plasma samples were removed from the incubator at their corresponding time point at 0, 1, 6, 24, 48 and 72 hours. 50 μL of each sample was quenched with 200 μL of cold acetonitrile containing internal standard (100 ng/mL of Dexamethasone). Samples were vortexed for 2 min then centrifuged at 4500 rpm for 15 min. The extraction supernatants were diluted with DI water prior to sample analysis.

Calibration standards were used to construct calibration curves for plasma samples. Linearity of the calibration curve was established from MRM peak area ratio of compounds, as a function of compound concentrations. The concentration-response relationship was linear in the concentration range from 0.5 to 2000 ng/mL. The sample was analyzed by an LC-MS/MS system consisting of an ExionLC AD HPLC system with a Phenomenex Kinetex 5 μm —C18 100A (2.1*50 mm) column using gradient elution and an REF TRIPLE QUAD 5500 mass spectrometer. The mass spectrometer is operated in the positive electrospray ionization (ESI) ion mode. Tables 12 and 13 show percentage of payload loss of iADCs in human and mouse plasma at various time points. It can be seen that the iADCs of the present disclosure show good stability in either mouse or human plasma.

TABLE 12
Percentage of payload loss of iADCs in
human and mouse plasma at various time points

| | Payload loss (%) in human plasma | | | | | Payload loss (%) in mouse plasma | | | | |
|---|---|---|---|---|---|---|---|---|---|---|
| iADCs | 0 h | 1 h | 6 h | 24 h | 48 h | 0 h | 1 h | 6 h | 24 h | 48 h |
| iADC-a0 | 1.01 | 3.52 | 12.7 | 32.5 | 47.9 | 0.820 | 8.40 | 16.9 | 28.4 | 39.6 |
| iADC-a5 | 0.480 | 0.530 | 1.50 | 6.47 | 9.52 | 0.390 | 1.37 | 4.34 | 8.32 | 13.6 |
| iADC-a21 | 0 | 0.11 | 0.18 | 1.67 | 11.2 | 0 | 1.02 | 2.66 | 2.45 | 8.80 |
| iADC-a27 | 0.050 | 0.12 | 0.400 | 2.34 | 14.88 | 0 | 2.42 | 5.06 | 9.44 | 25.6 |
| iADC-a28 | 0.00 | 0.00 | 1.46 | 5.94 | 13.4 | 0.230 | 0.310 | 0.780 | 1.97 | 4.41 |
| iADC-a31 | 0 | 0.080 | 0.280 | 3.55 | 14.3 | 0 | 1.99 | 4.14 | 6.14 | 27.0 |

TABLE 13
Percentage of payload loss of iADCs in human and mouse plasma
48 h after co-incubation

| | Payload loss (%) | Payload loss (%) |
|---|---|---|
| iADCs | in human plasma | in mouse plasma |
| iADC-a32 | 8.00 | 7.54 |
| iADC-a33 | 5.32 | 6.30 |
| iADC-a35 | 27.3 | 21.4 |
| iADC-a36 | 6.88 | 5.87 |
| iADC-a37 | 19.86 | 14.95 |
| iADC-a38 | 1.53 | 4.25 |
| iADC-a39 | 9.15 | 6.04 |
| iADC-a40 | 8.5 | 6.48 |

### Example VI-4 In Vivo Assay

This study is to examine the anti-tumor efficacy of test compounds in SK—OV-3 (HER2-high expression) ovarian cancer xenograft model in CB17-SCID mice. Each mouse was inoculated subcutaneously at the right flank region with SK—OV-3 (1×10⁷) cells in 0.1 mL of PBS and Matrigel (1:1) for tumor development. The mice were randomized according to tumor size and weight, the treatment began when the mean tumor size reached 100-200 mm³. The iADCs and trastuzumab were administered at a dose of 1 mg/kg (i.v. once), and the corresponding payload was administered at a dose of 0.04 mg/kg (i.v. once). Anti-tumor activity was assessed according to relative tumor inhibition rate (TGI), and safety was assessed according to changes in body weight, drug withdrawal, and death.

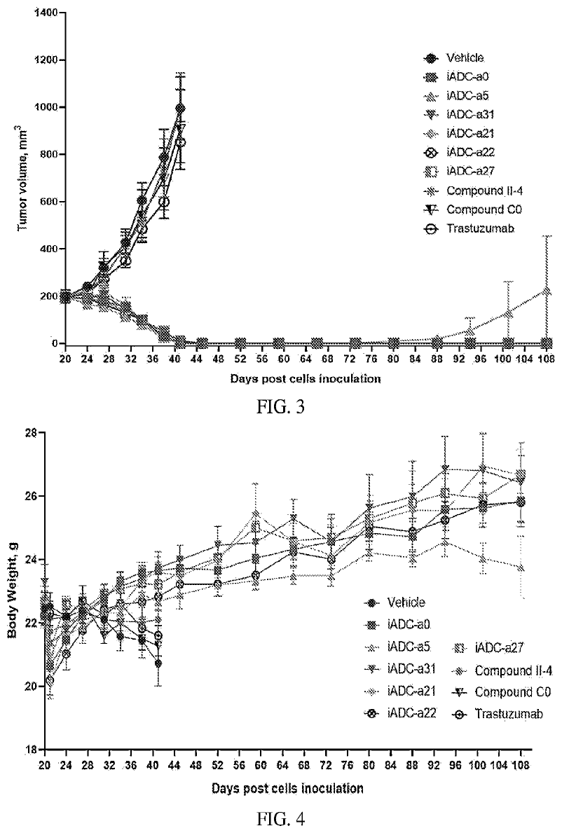

Tumor volumes were measured in two dimensions using a caliper, and the volume was expressed in mm³ using the formula: V=0.5 a×b² where a and b are the long and short diameters of the tumor, respectively. The results were shown in FIGS. 3-4.

What is claimed is:

1. A compound-linker construct, wherein the compound-linker construct is of Formula S3:
T^{B}ₘ-D-L¹ (S3)
or a pharmaceutically acceptable salt or solvate thereof, wherein:
D is a STING agonist moiety;
L¹ is a linker;
T^{B} is a hydrophilic group; and
m is O or 1.

2. The compound-linker construct of claim 1, wherein the compound-linker construct is of Formula S2:
D-L¹ (S2)
or a pharmaceutically acceptable salt or solvate thereof.

3. A conjugate of Formula S0:
[T^{B}ₘ-D-L¹]_{d5}-T (S0)
or a pharmaceutically acceptable salt or solvate thereof, wherein:
(T^{B}ₘ-D-L¹) is the compound-linker construct of claim 1;
T is a targeting moiety; and
d5 is an integer from 1 to 20.

4. The conjugate of claim 1, wherein the conjugate has the structure of Formula S1:
(D-L1)_{d5}-T (S1)
or a pharmaceutically acceptable salt or solvate thereof.

5. The compound-linker construct or conjugate of any one of the preceding claims, wherein T^{B} when present, is a hydrophilic group, preferably selected from the group consisting of: polyalcohols, polyethers, polyanions, polycations, polyphosphoric acids, polyamines, polysaccharides, polyhydroxy compounds, polylysines, and derivatives thereof.

6. The compound-linker construct or conjugate of any one of the preceding claims, wherein L¹ has the structure of H^{1A}-L^{C}-H^{1B} L^{C} is 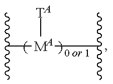
wherein
M^{A} when present, is an amino acid residue or a peptide moiety comprising at least two amino acids residue;
T^{A} when present, is a hydrophilic group.

7. The compound-linker construct or conjugate of any one of the preceding claims, being of Formula S1b: 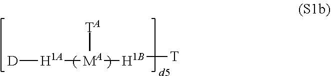
or a pharmaceutically acceptable salt or solvate thereof.

8. The compound-linker construct or conjugate of any one of the preceding claims, wherein the STING agonist moiety [D] is a compound of Formula Y-1, Y-2, Y-3, or an ester, stereoisomer, tautomer, isotopic derivative, prodrug or pharmaceutically acceptable salt thereof: 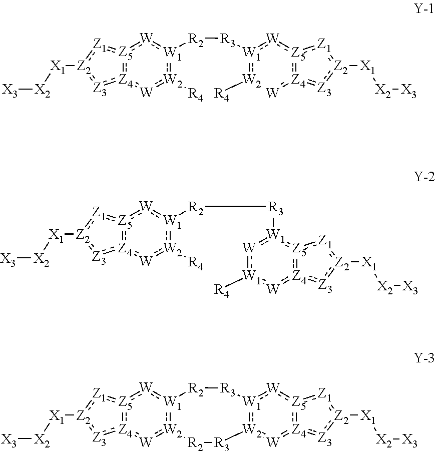
or pharmaceutically acceptable salts thereof, wherein, [symbol]
represents a single bond or a double bond;
each W is independently selected from CR₁, C(R₁)₂, N, NR₁, O or S;
each W₁ is independently selected from C, CR₁, or N;
each W₂ is independently selected from C, CR₁, or N;
each Z₁ is independently selected from CR₁, C(R₁)₂, N, NR₁, O or S;
each Z₂ is independently selected from CR₁, C or N;
each Z₃ is independently selected from CR₁, C(R₁)₂, N, NR₁, O or S;
each Z₄ is independently selected from C, CR₁ or N;
each Z_{W} is independently selected from C, CR₁ or N;
each R₁ is independently selected from H, deuterium, halogen, OR, N(R⁶)₂, COOR⁶, or C(O)N(R⁶)₂, CN or C₁-C₆ alkyl; wherein the C₁-C₆ alkyl is optionally substituted with one or more deuterium, halogen, OR, NO₂, COOR, or C(O)N(RN)₂;
R₂ and R₃ are independently selected from the group consisting of O—(C₁-C₄ alkylene or haloalkylene), C₁-C₅ alkylene or haloalkylene, N(R⁶)—(C₁-C₄ alkylene or haloalkylene), -Tₐ-C₁-C₆ alkyl-T_{b}-, -Tₐ- N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}-, -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃-C₁₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ- (C₃-C₁₂cycloalkyl)-T_{b}-, -Tₐ- (C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs;
PEGₙ is (—OCH₂CH₂—)ₙ, n=1-16, preferably 1-8;
Tₐ and T_{b} each independently are absent, —N(Rs)-, -o-, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O— (C₁-C₆ alkyl)-O—; wherein the C₁-C₆ alkyl is optionally substituted with one or more halogen; and
each Rs independently is H, deuterium or C₁-C₆ alkyl optionally substituted with one or more halogen;
each R⁴ is independently selected from the group consisting of H, deuterium, halogen, CN, OR⁶, N(R⁶)₂, COOR⁶, C(O)N(R⁶)₂, SO₂R⁶, C₁-C₆ alkyl, C₁-C₆ haloalkyl, C₁-C₆ alkyl substituted by OR⁶, C₂-C₆ alkenyl, C₂-C₆ haloalkenyl, C₂-C₆ alkenyl substituted by OR⁶, C₂-C₆ alkynyl, C₂-C₆ haloalkynyl, C₂-C₆ alkynyl substituted by OR⁶, C₃-C₆ cycloalkyl, and a 3- to 6-membered heterocyclic ring including 1 to 2 ring members selected from the group consisting of O, S, and N(R⁶);
each R⁶ is independently selected from the group consisting of —H, deuterium, halogen, —NH₂, —CN, —OH, —N₃, —NO₂, carboxyl, C₁₋₆alkyl, C₁₋₆alkoxy, C₂₋₆alkenyl, C₂₋₆alkynyl, —C₆₋₁₀aryl, —C₅₋₁₀heteroaryl, C₃₋₁₀heterocyclic ring or C₃₋₁₀carbocyclic ring; and each of which is independently optionally substituted with deuterium, halogen, —NH₂, —CN, —OH, —NO₂, carbonyl, ═O, oxo, carboxyl, C₁₋₆alkoxy, or C₁₋₆alkyl; and each of the heteroaryl and heterocyclic ring contains at least one heteroatoms selected from N, O or S;
each X1 is independently selected from the group consisting of C═O, —CH₂—, —CHF—, and —CF₂—;
each X2 is independently selected from (C(R⁸)₂)(₁_3), NR(C(R⁸)₂)(₁_3), —NH(C(R⁸)₂)(₁_3), —N(C₁₋₆alkyl)(C(R⁸)₂)₍₁₋₃₎ or —N(haloC₁₋₆alkyl) (C(R⁸)₂)₍₁₋₃₎; wherein each R⁸ is independently selected from the group consisting of H, deuterium, halogen, C₁-C₆ alkyl, CN, OR⁶, N(R⁶)₂, C₁-C₆ haloalkyl, C₃-C₆ cycloalkyl, C₁-C₆ alkyl substituted by OR⁶, and C₁-C₆ alkyl substituted by N(R⁶)₂; optionally 2 R⁸ on different carbon atoms may be taken together, along with the atoms to which they are attached, to form a 3- to 6-membered fused ring; and
optionally 2 R⁸ on a single carbon atom may be taken together, along with the atom to which they are attached, to form a 3- to 6-membered spirocycle;
each X3 is independently selected from the group consisting of H, CN, COOR⁶, C(O)SR⁶, C(S)OR⁶, SO₂R⁶, C(O)N(R₉)₂, 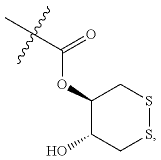 [SMILES: *C(=O)O[C@H]1CSSC[C@@H]1O]
OR⁶, SR⁶, N(R⁶)₂, OCOR⁶, NR⁶COR⁶, C₁₋₆alkyl, C₁₋₆alkoxy, C₂₋₆alkenyl, C₂₋₆alkynyl, —C₆₋₁₀aryl, —C₅₋₁₀heteroaryl, C₃₋₁₀heterocyclic ring or C₃₋₁₀carbocyclic ring; and each R⁹ is independently selected from H, deuterium, COOR⁶, SO₂R⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆alkyl), S(C₆-C₁₀ aryl), S(═O)₂R⁶, S(═O)₂O R⁶, P(═O)(R⁶)₂, C₁-C₆alkyl, C₂-C₆ alkenyl, C₂-C₆ alkynyl, C₃-C₅ cycloalkyl, C₆-C₁₀aryl, 3-8 membered heterocycloalkyl, or 3-10 membered heteroaryl.

9. The compound-linker construct or conjugate of any one of the preceding claims, wherein 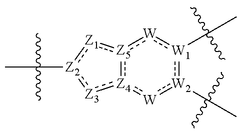
is independently selected from

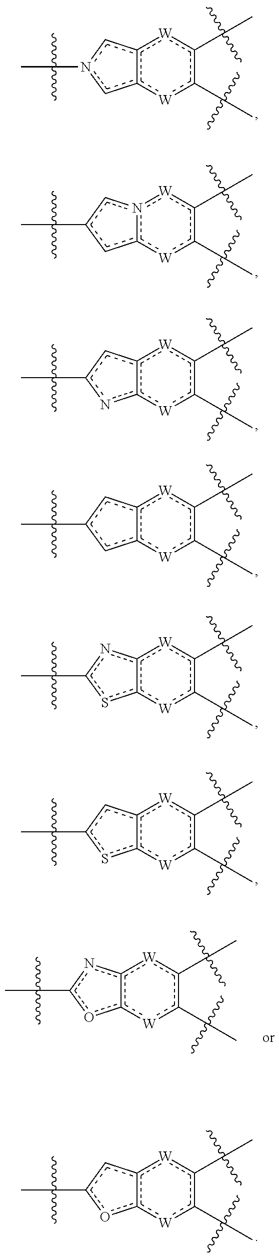

10. The compound-linker construct or conjugate of any one of the preceding claims, wherein 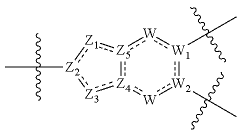
is independently selected from

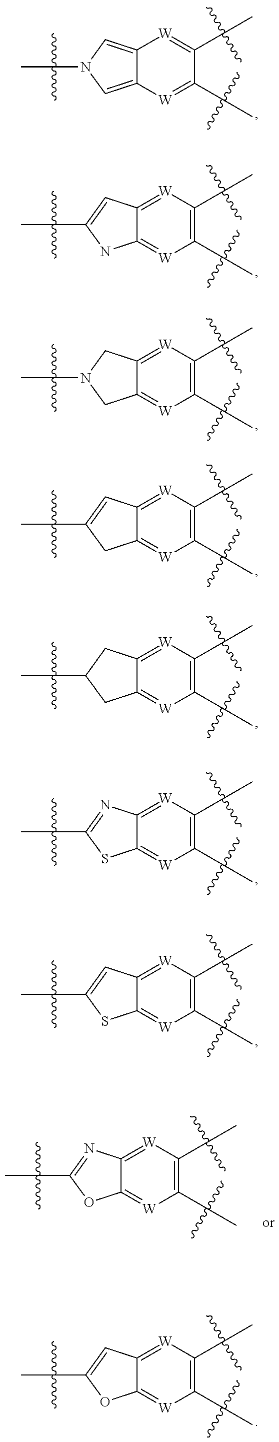

11. The compound-linker construct or conjugate of any one of the preceding claims, wherein 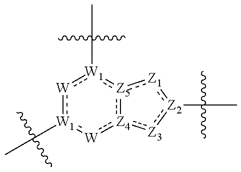
is independently selected from

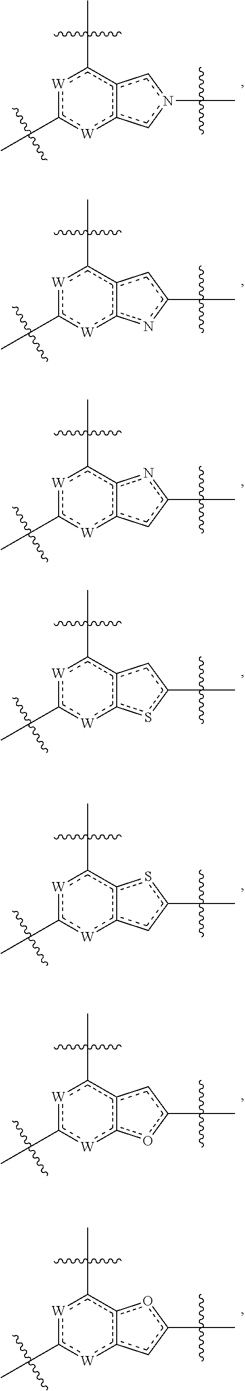

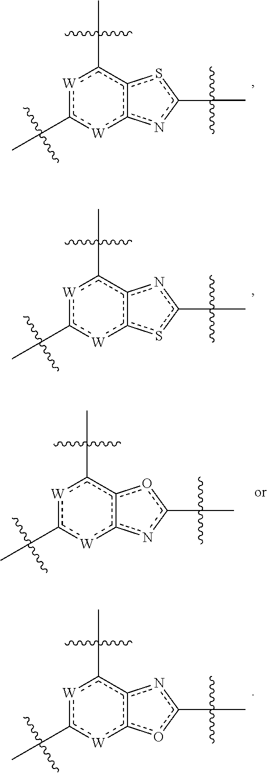

12. The compound-linker construct or conjugate of any one of the preceding claims, wherein 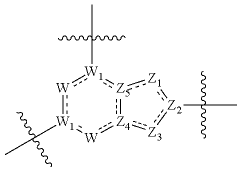
is independently selected from

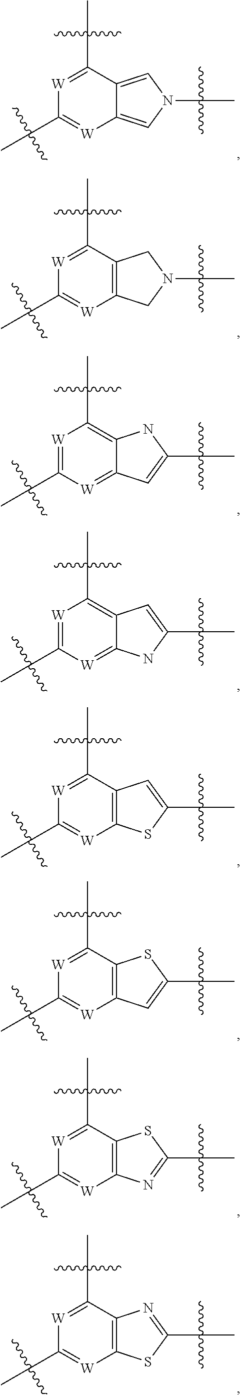

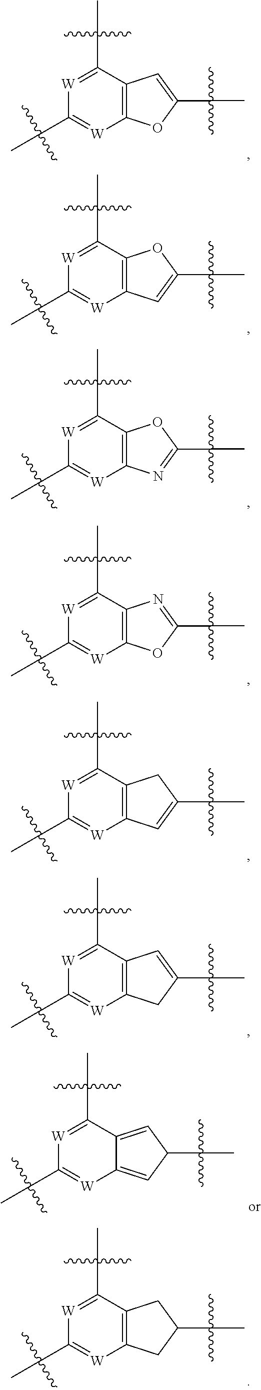

13. The compound-linker construct or conjugate of any one of the preceding claims, wherein D is a compound of Formula (A), (B), (C), or an ester, stereoisomer, tautomer, isotopic derivative, prodrug, or pharmaceutically acceptable salt thereof:
wherein 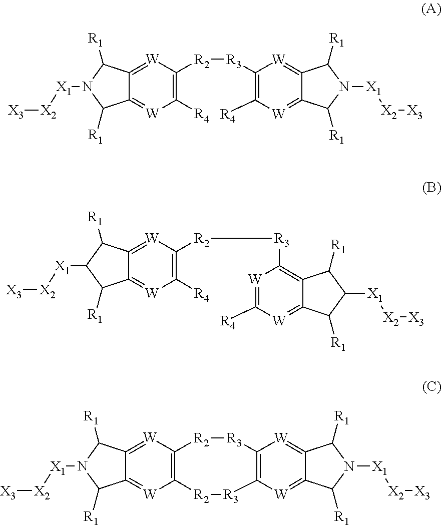
each W is independently selected from CR₁ or N;
each R₁ is independently selected from H, deuterium, halogen, OR⁶, N(R⁶)₂, COOR⁶, or C(O)N(R⁶)₂, CN or C₁-C₆ alkyl; wherein the C₁-C₆ alkyl is optionally substituted with one or more deuterium, halogen, OR⁶, N(R⁶)₂, COOR⁶, or C(O)N(R⁶)₂;
R₂ and R₃ are independently selected from the group consisting of O—(C₁-C₄ alkylene or haloalkylene), C₁-C₅ alkylene or haloalkylene, N(R⁶)—(C₁-C₄ alkylene or haloalkylene), -Tₐ-C₁-C₆ alkyl-T_{b}-, -Tₐ- N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}-, - Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃—C₁₋₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-Te—, -Tₐ- (C₃-C₁₂cycloalkyl)-T-, -Tₐ- (C₆-C₁₂ aryl)-T-, -Tₐ-(3- to 12-membered heterocycloalkyl)-T_{b}-, or -Tₐ-(5- to 12-membered heteroaryl)-Te—, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O— (C₁-C₆ alkyl)-O—; wherein the C₁-C₆ alkyl is optionally substituted with one or more halogen; and
each Rs independently is H, deuterium or C₁-C₆ alkyl optionally substituted with one or more halogen;
each R₄ is independently selected from the group consisting of H, deuterium, halogen, CN, OR⁶, N(R⁶)₂, COOR⁶, C(O)N(R⁶)₂, SO₂R⁶, C₁-C₆ alkyl, C₁-C₆ haloalkyl, C₁-C₆ alkyl substituted by OR⁶, C₂-C₆ alkenyl, C₂-C₆ haloalkenyl, C₂-C₆ alkenyl substituted by OR⁶, C₂-C₆ alkynyl, C₂-C₆ haloalkynyl, C₂-C₆ alkynyl substituted by OR⁶, C₃-C₆ cycloalkyl, and a 3- to 6-membered heterocyclic ring including 1 to 2 ring members selected from the group consisting of O, S, and N(R⁶);
each R⁶ is independently selected from the group consisting of —H, deuterium, halogen, —NH₂, —CN, —OH, —N₃, —NO₂, carboxyl, C₁₋₆alkyl, C₁₋₆alkoxy, C₂₋₆alkenyl, C₂₋₆alkynyl, —C₆₋₁₀aryl, —C₅₋₁₀heteroaryl, C₃₋₁₀heterocyclic ring or C₃₋₁₀carbocyclic ring; and each of which is independently optionally substituted with deuterium, halogen, —NH₂, —CN, —OH, —NO₂, carbonyl, ═O, oxo, carboxyl, C₁₋₆alkoxy, or C₁₋₆alkyl; and each of the heteroaryl and heterocyclic ring contains at least one heteroatoms selected from N, O or S;
each X₁ is independently selected from the group consisting of C═O, —CH₂—, —CHF—, and —CF₂—;
each X₂ is independently selected from (C(R⁸)₂)₍₁₋₃₎, —NH(C(R⁸)₂)₍₁₋₃₎, —N(C₁₋₆alkyl) (C(R⁸)₂)₍₁₋₃₎ or —N(haloC₁₋₆alkyl) (C(R⁸)₂)₍₁₋₃₎; wherein each R⁸ is independently selected from the group consisting of H, deuterium, halogen, C₁-C₆ alkyl, CN, OR⁶, N(R⁶)₂, C₁-C₆ haloalkyl, C₃-C₆ cycloalkyl, C₁-C₆ alkyl substituted by OR⁶, and C₁-C₆ alkyl substituted by N(R⁶)₂; optionally 2 R⁸ on different carbon atoms may be taken together, along with the atoms to which they are attached, to form a 3- to 6-membered fused ring; and optionally 2 R⁸ on a single carbon atom may be taken together, along with the atom to which they are attached, to form a 3- to 6-membered spirocycle;
each X₃ is independently selected from the group consisting of COOR⁶, C(O)SR⁶, C(S)OR⁶, SO₂R⁶, C(O)N(R⁹)₂, 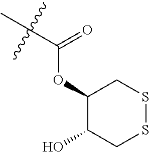 [SMILES: *C(=O)O[C@H]1CSSC[C@@H]1O]
and CN; and each R⁹ is independently selected from H, deuterium, COOR⁶, SO₂R⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆alkyl), S(C₆-C₁₀ aryl), S(═O)₂R⁶, S(═O)₂OR⁶, P(═O)(R⁶)₂, C₁-C₆alkyl, C₂-C₆ alkenyl, C₂-C₆ alkynyl, C₃-C₅ cycloalkyl, C₆-C₁₀aryl, 3-8 membered heterocycloalkyl, or 3-10 membered heteroaryl.

14. The compound-linker construct or conjugate of any one of the preceding claims, wherein D is a compound of Formula I, II, III, IV, or V, or an ester, stereoisomer, tautomer, isotopic derivative, prodrug, or pharmaceutically acceptable salt thereof: 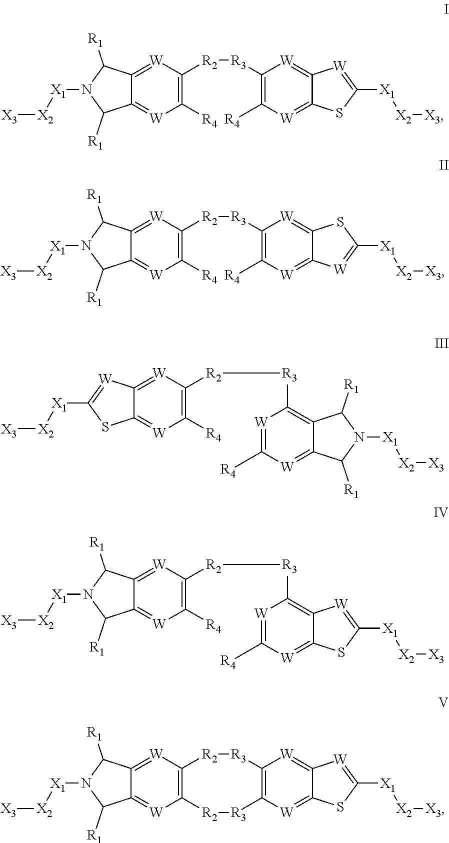
wherein:
each W is independently selected from CR₁ or N;
each R₁ is independently selected from H, deuterium, halogen, OR⁶, N(R⁶)₂, COOR⁶, or C(O)N(R⁶)₂, CN or C₁-C₆ alkyl; wherein the C₁-C₆ alkyl is optionally substituted with one or more deuterium, halogen, OR⁶, N(R⁶)₂, COOR⁶, or C(O)N(R⁶)₂;
R₂ and R₃ are independently selected from O—(C₁-C₄ alkylene or haloalkylene), C₁-C₅ alkylene or haloalkylene, N(R⁶)—(C₁-C₄ alkylene or haloalkylene), -Tₐ-C₁-C₆ alkyl-T_{b}-, -Tₐ- N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ- N(R₅)—N(Rs)-T_{b}-, - Tₐ-C₂-C₆alkenyl-T_{b}-, -Tₐ-C₂-C₆alkynyl-T_{b}-, -Tₐ-C(═O)-T_{b}-, -Tₐ-C(=CH₂)-T_{b}-, -Tₐ-C(═O)—C(═O)-T-, -Tₐ-C(═O)—(C₁-C₆alkyl)-C(═O)-T_{b}-, -Tₐ-C(═O)—(C₃-C₁₂cycloalkyl)-C(═O)-T-, -Tₐ-C(=)—(C-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ- (C₃-C₁₂cycloalkyl)-T_{b}-, -Tₐ- (C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O— (C₁-C₆ alkyl)-O—; wherein the C₁-C₆ alkyl is optionally substituted with one or more halogen; and
each Rs independently is H, deuterium or C₁-C₆ alkyl optionally substituted with one or more halogen;
each R₄ is independently selected from the group consisting of H, deuterium, halogen, CN, OR⁶, N(R⁶)₂, COOR⁶, C(O)N(R⁶)₂, SO₂R⁶, C₁-C₆ alkyl, C₁-C₆ haloalkyl, C₁-C₆ alkyl substituted by OR⁶, C₂-C₆ alkenyl, C₂-C₆ haloalkenyl, C₂-C₆ alkenyl substituted by OR⁶, C₂-C₆ alkynyl, C₂-C₆ haloalkynyl, C₂-C₆ alkynyl substituted by OR⁶, C₃-C₆ cycloalkyl, and a 3- to 6-membered heterocyclic ring including 1 to 2 ring members selected from the group consisting of O, S, and N(R⁶);
each R⁶ is independently selected from the group consisting of —H, deuterium, halogen, —NH₂, —CN, —OH, —N₃, —NO₂, carboxyl, C₁₋₆alkyl, C₁₋₆alkoxy, C₂₋₆alkenyl, C₂₋₆alkynyl, —C₆₋₁₀aryl, —C₅₋₁₀heteroaryl, C₃₋₁₀heterocyclic ring or C₃₋₁₀carbocyclic ring; and each of which is independently optionally substituted with deuterium, halogen, —NH₂, —CN, —OH, —NO₂, carbonyl, ═O, oxo, carboxyl, C₁₋₆alkoxy, or C₁₋₆alkyl; and each of the heteroaryl and heterocyclic ring contains at least one heteroatoms selected from N, O or S;
each X₁ is independently selected from the group consisting of C═O, —CH₂—, —CHF—, and —CF₂—;
each X₂ is independently selected from (C(R⁸)₂)₍₁₋₃₎, —NH(C(R⁸)₂)₍₁₋₃₎, —N(C₁₋₆alkyl) (C(R⁸)₂)₍₁₋₃₎ or —N(haloC₁₋₆alkyl) (C(R⁸)₂)₍₁₋₃₎; wherein each R⁸ is independently selected from the group consisting of H, deuterium, halogen, C₁-C₆ alkyl, CN, OR⁶, N(R⁶)₂, C₁-C₆ haloalkyl, C₃-C₆ cycloalkyl, C₁-C₆ alkyl substituted by OR⁶, and C₁-C₆ alkyl substituted by N(R⁶)₂; optionally 2 R⁸ on different carbon atoms may be taken together, along with the atoms to which they are attached, to form a 3- to 6-membered fused ring; and optionally 2 R⁸ on a single carbon atom may be taken together, along with the atom to which they are attached, to form a 3- to 6-membered spirocycle;
each X₃ is independently selected from the group consisting of COOR⁶, C(O)SR⁶, C(S)OR⁶, SO₂R⁶, C(O)N(R⁹)₂, 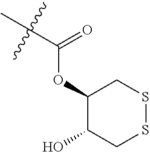 [SMILES: *C(=O)O[C@H]1CSSC[C@@H]1O]
and CN; and each R⁹ is independently selected from H, deuterium, COOR⁶, SO₂R⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆alkyl), S(C₆-C₁₀ aryl), S(═O)₂R⁶, S(═O)₂OR⁶, P(═O)(R⁶)₂, C₁-C₆alkyl, C₂-C₆ alkenyl, C₂-C₆ alkynyl, C₃-C₅ cycloalkyl, C₆-C₁₀aryl, 3-8 membered heterocycloalkyl, or 3-10 membered heteroaryl.

15. The compound-linker construct or conjugate of any one of the preceding claims, wherein each D is of Formula (A), 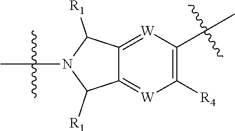
is independently selected from 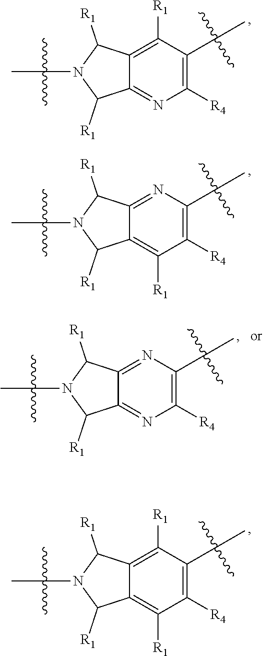
R¹⁻¹⁻¹
each R₁, R₂, R₃, R⁴, X₁, X₂ and X₃ are as defined in claim 13 or 14.

16. The compound-linker construct or conjugate of any one of the preceding claims, wherein each D is of Formula (B), 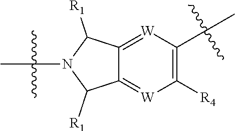
is independently selected from 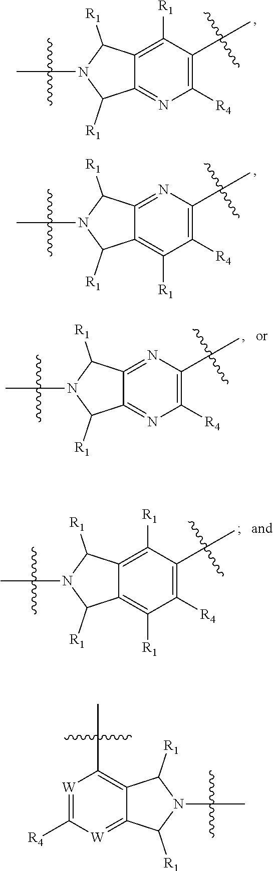
is independently selected from 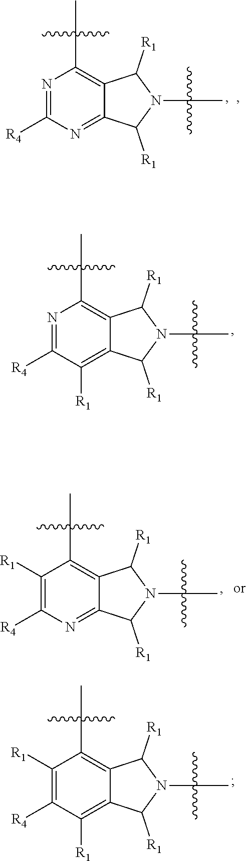
each R₁, R₂, R₃, R₄, X₁, X₂ and X₃ are as defined in claim 13 or 14.

17. The compound-linker construct or conjugate of any one of the preceding claims, wherein each D is of Formula (C), 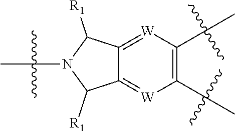
is independently selected from 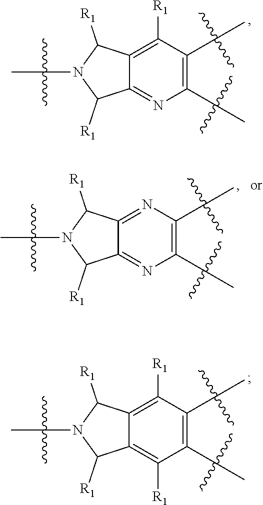
each R₁, R₂, R₃, R⁴, X₁, X₂ and X₃ are as defined in claim 13 or 14.

18. The compound-linker construct or conjugate of any one of the preceding claims, wherein each D is of Formula I, 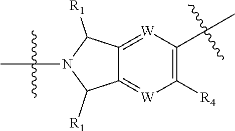
is independently selected from 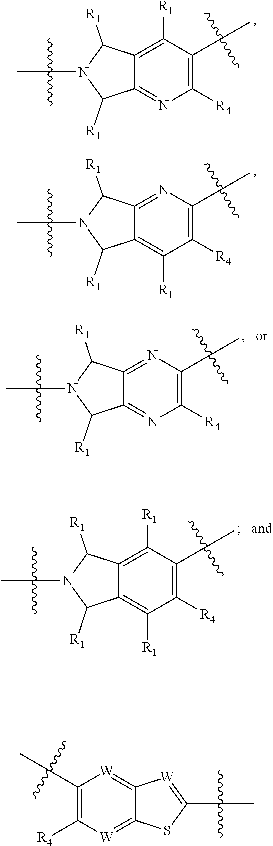
is independently selected from 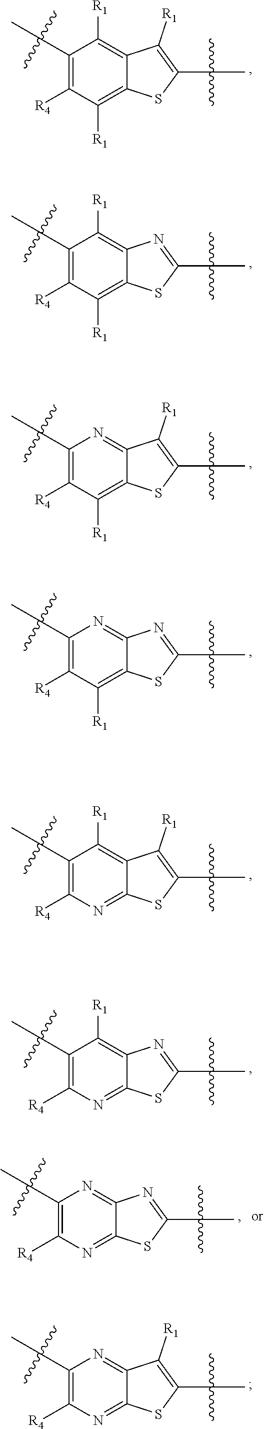
each R₁, R², R³, R₄, X₁, X₂ and X₃ are as defined in claim 13 or 14.

19. The compound-linker construct or conjugate of any one of the preceding claims, wherein each D is of Formula II, 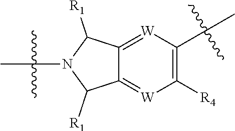
is independently selected from 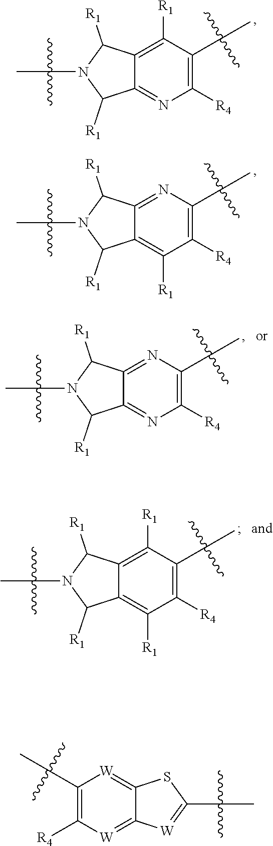
is independently selected from 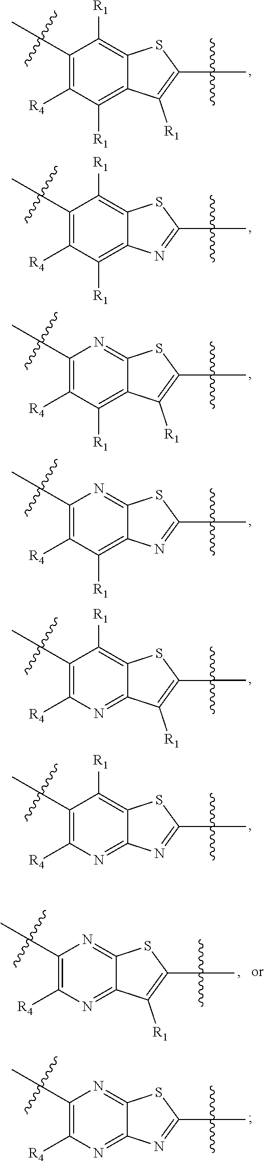
each R₁, R₂, R₃, R₄, X₁, X₂ and X₃ are as defined in claim 13 or 14.

20. The compound-linker construct or conjugate of any one of the preceding claims, wherein each D is of Formula III, 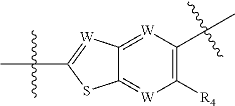
is independently selected from 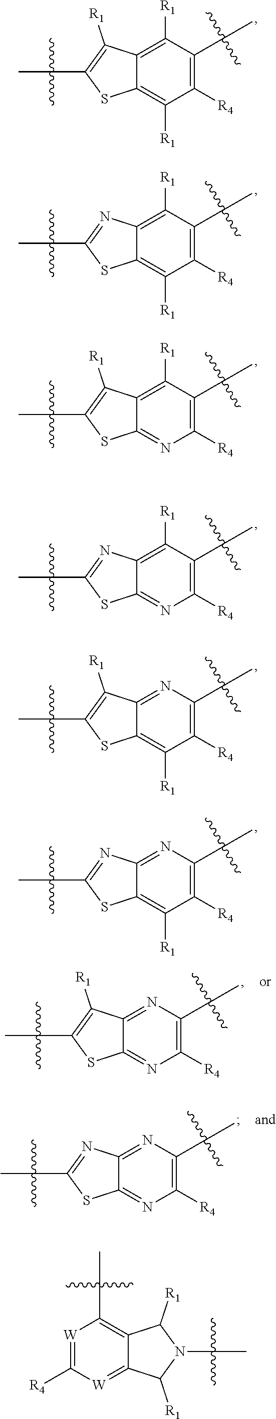
is independently selected from 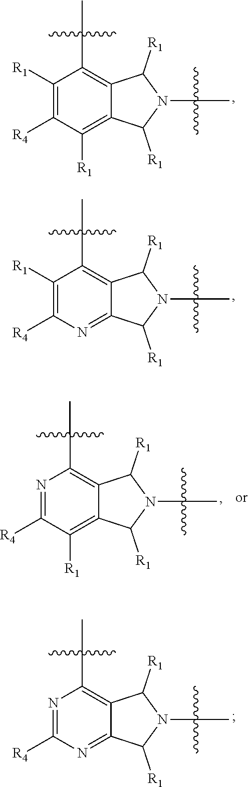
each R₁, R₂, R₃, R₄, X₁, X₂ and X₃ are as defined in claim 13 or 14.

21. The compound-linker construct or conjugate of any one of the preceding claims, wherein each D is of Formula IV, 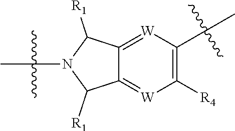
is independently selected from 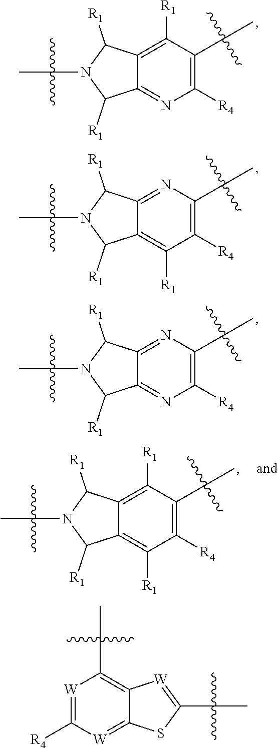
is independently selected from 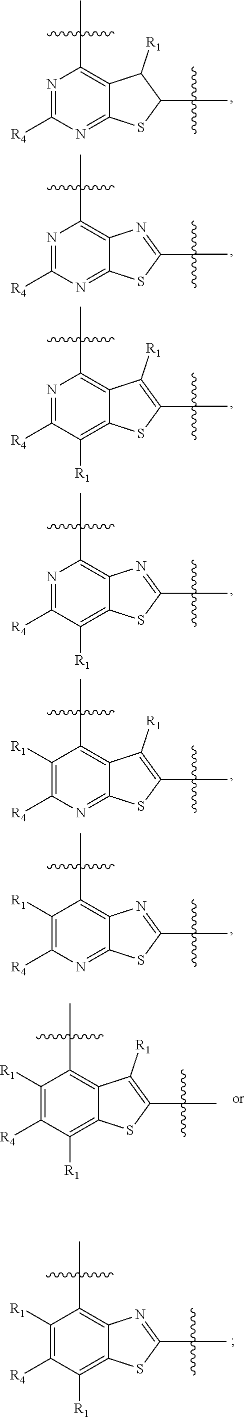
each R₁, R₂, R₃, R₄, X₁, X₂ and X₃ are as defined in claim 13 or 14.

22. The compound-linker construct or conjugate of any one of the preceding claims, wherein each D is of Formula V, 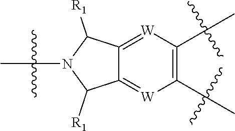
is independently selected from 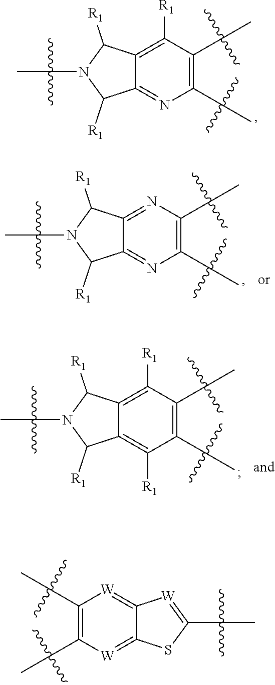
is independently selected from 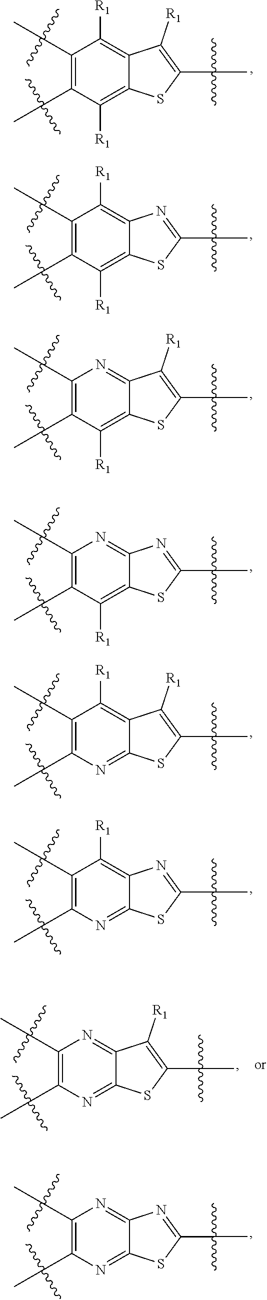
each R₁, R₂, R₃, R₄, X₁, X₂ and X₃ are as defined in claim 13 or 14.

23. The compound-linker construct or conjugate of any one of the preceding claims, wherein each D independently is selected from compounds described in Table 1.

24. The compound-linker construct or conjugate of any one of the preceding claims, wherein each D independently is selected from compounds described in Table 2.

25. The compound-linker construct or conjugate of any one of the preceding claims, wherein a covalent bond between the compound of formula Y-1, Y-2, Y-3, (A), (B), (C), I, II, III, IV, or V and the linker L1 is established by the reaction of a functional group of the compound of formula Y-1, Y-2, Y-3, (A), (B), (C), I, II, III, IV, or V with a functional group handle of the linker Li; and wherein preferably the functional group of the compound of formula Y-1, Y-2, Y-3, (A), (B), (C), I, II, III, IV, or V is attached to or part of the substituents R₁, R₂, R₃, R₄, X₁, X₂, or X₃ so that the linker L1 will be covalently bonded to the compound of formula Y-1, Y-2, Y-3, (A), (B), (C), I, II, III, IV, or V.

26. The compound-linker construct or conjugate of any one of claims 1-25, wherein the isotopic derivative is a deuterated derivative.

27. The compound-linker construct or conjugate of any one of the preceding claims, wherein the linker L¹ is a linker comprising one or more cleavage elements, and each cleavage element is independently selected from a self-immolative spacer and a group that is susceptible to cleavage.

28. The compound-linker construct or conjugate of any one of the preceding claims, wherein the cleavage is selected from acid-induced cleavage, peptidase-induced cleavage, esterase-induced cleavage, glycosidase-induced cleavage, phosphodiesterase-induced cleavage, phosphatase-induced cleavage, protease-induced cleavage, lipase-induced cleavage, or disulfide bond cleavage.

29. The compound-linker construct or conjugate of any one of the preceding claims, wherein the linker L1 comprises:
(i) a chain L^{c} of 2 to 100 atoms selected from carbon, nitrogen, oxygen, and sulfur atoms, which may be interrupted by 5- to 10-membered aryl and heteroaryl groups and/or 3- to 8-membered saturated carbocyclyl or heterocyclyl groups, wherein the aforementioned heteroaryl and heterocyclic groups comprise one or more, same or different heteroatoms selected from O, N or S, wherein said N- and/or S-atoms are independently oxidized or non-oxidized, and wherein each substitutable carbon or heteroatom in the chain is independently unsubstituted or substituted with one or more same or different substituents selected from halogen, OH, ═O, C₁-C₆-alkyl, C₁-C₆-haloalkyl, C₃-C₆-cycloalkyl, C₃-C₆-halocycloalkyl, C₁-C₆-alkoxy, and C₁-C₆-haloalkoxy;
and preferably
a chain L^{c} of units selected from a linear or branched polyethylene glycol chain, a sequence of amino acids, and a linear or branched C₁-C₁₀-alkyl chain, wherein each substitutable carbon or heteroatom of the aforementioned units may be unsubstituted or substituted with one or more, same or different substituents selected from halogen, OH, ═O, C₁-C₆-alkyl, C₁-C₆-haloalkyl, C₃-C₆-cycloalkyl, C₃-C₆-halocycloalkyl, C₁-C₆-alkoxy, and C₁-C₆-haloalkoxy;
(ii) a functional group handle H^{1A}, which is covalently bonded to the compound of formula (A), (B), (C), I, II, III, IV, or V;
(iii) a functional group handle H^{1B} suitable for forming a covalent bond to a targeting moiety T.

30. The compound-linker construct of any one of the preceding claims, being formula S2a:
D-H^{1A}-L^{C}-H^{1B} (S2a),
wherein H^{1B} is a monovalent linker moiety comprising a functional group capable of forming a covalent bond to a targeting moiety T;
D is the same as defined in any one of claims 13-24.

31. The compound-linker construct or conjugate of any one of the preceding claims, wherein L^{c} is 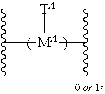
wherein
M^{A} when present, is an amino acid residue or a peptide moiety comprising at least two amino acids;
T^{A} when present, is a hydrophilic group.

32. The compound-linker construct or conjugate of any one of the preceding claims, wherein in the conjugates, H^{1B} is selected from the group consisting of 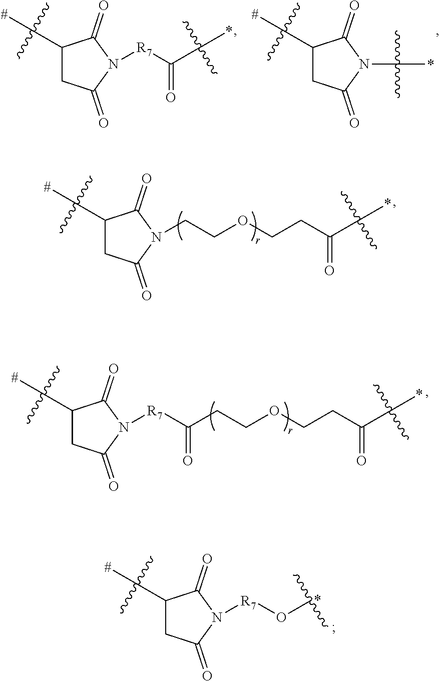
wherein in the compound-linker constructs, H^{1B} is selected from the group consisting of 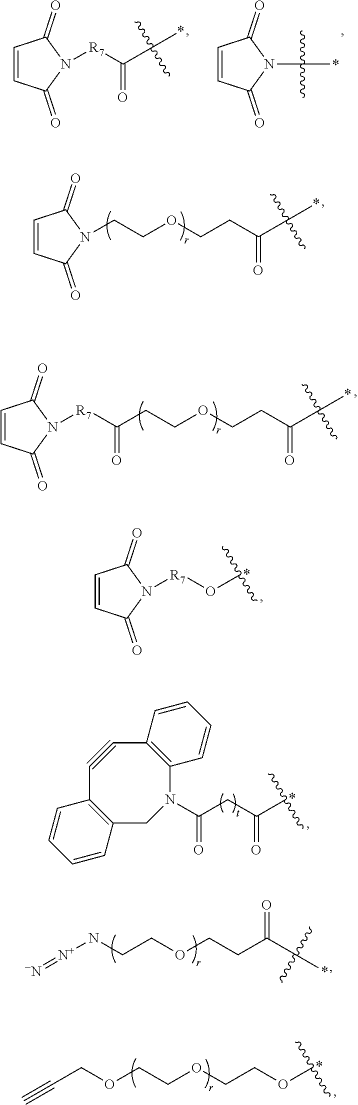
and wherein:
R₇ is —O—, —NR^{7a}, —(C₁-C₁₀ alkyl)-, —(C₁-C₁₀ alkenyl)-, —(C₁-C₁₀ alkynyl)-, —(C₃-C₅ cycloalkyl)-, -aryl-, —O—(CI—Cs alkyl)-, —O—(C₁-C₁₀ alkenyl)-, —O—(C₁-C₁₀ alkynyl)-, —(C₁-C₁₀ alkyl)-(C₃-C₅ cycloalkyl)-, —(C₁-C₁₀ alkyl)-aryl-, —(C₂-C₁₀ alkenyl)-(C₃-C₅ cycloalkyl)-, —(C₂-C₁₀ alkenyl)-aryl-, —(C₂-C₁₀ alkynyl)-(C₃-C₅ cycloalkyl)-, —(C₂-C₁₀ alkynyl)-aryl-, —(C₃-C₅ cycloalkyl)-(C₁-C₁₀alky)-, -aryl-(C₁-C₁₀alky)-, —(C₃-C₅cycloalkyl)-(C₂-C₁₀ alkenyl)-, -aryl-(C₂-C₁₀ alkenyl)-, —(C₃-C₅ cycloalkyl)-(C₂-C₁₀ alkynyl)-, -aryl-(C₂-C₁₀ alkynyl)-, -(3- to 8-membered heterocycloalkyl)-, -(5- to 8-membered heteroaryl)-, —(C₁-C₁₀ alkyl)-(3- to 8-membered heterocycloalkyl)-, —(C₁-C₁₀ alkyl)-(5- to 8-membered heteroaryl)-, —(C₂-C₁₀ alkenyl)-(3- to 8-membered heterocycloalkyl)-, —(C₂-C₁₀ alkenyl)-(5- to 8-membered heteroaryl)-, —(C₂-C₁₀ alkynyl)-(3- to 8-membered heterocycloalkyl)-, —(C₂-C₁₀ alkynyl)-(5- to 8-membered heteroaryl)-, -(3- to 8-membered heterocycloalkyl)-(C₁-C₁₀ alkyl)-, -(5- to 8-membered heteroaryl)-(C₁-C₁₀ alkyl)-, -(3- to 8-membered heterocycloalkyl)-(C₂-C₁₀ alkenyl)-, -(5- to 8-membered heteroaryl)-(C₂-C₁₀ alkenyl)-, -(5- to 8-membered heteroaryl)-(C₂-C₁₀ alknyl)-, -(5- to 8-membered heteroaryl)-(C₂-C₁₀ alknyl)-, —O—C(O)—(CH₂CH₂O)ᵣ—(CH₂)₂—, —(CH₂CH₂O)ᵣ—, —(CH₂CH₂O)ᵣ (CH₂)₂—or —CH(CH₂NH₂)—, wherein the alkyl, alkenyl, alkynyl, cycloalkyl, aryl, heterocycloalkyl, or heteroaryl is optionally substituted;
R^{7a} is H, hydroxy, or C_{1 4} alkyl;
each r is independently an integer ranging from about 1 to about 12;
t is an integer ranging from about 1 to about 8; and
# denotes attachment to T and * denotes attachment to L^{C}.

33. The compound-linker construct or conjugate of any one of the preceding claims, wherein in the conjugates, H^{1B} is selected from the group consisting of 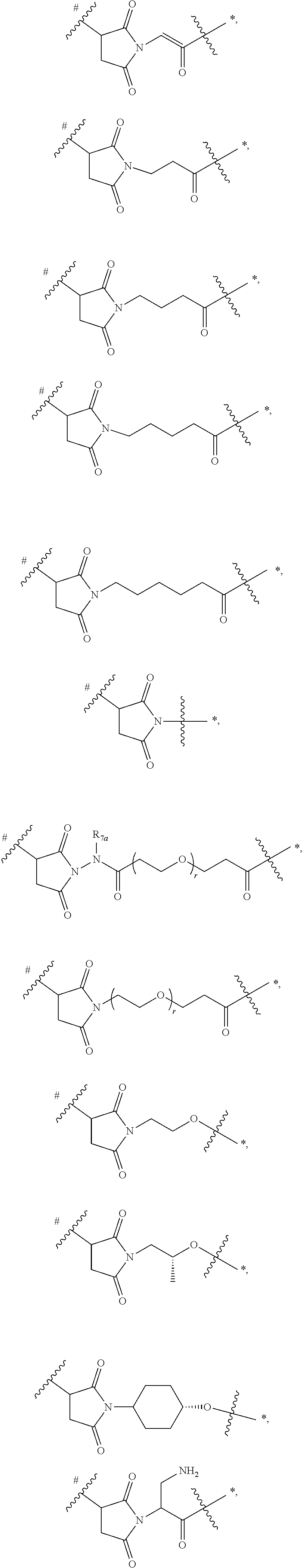
wherein in the compound-linker constructs, H^{1B} is selected from the group consisting of 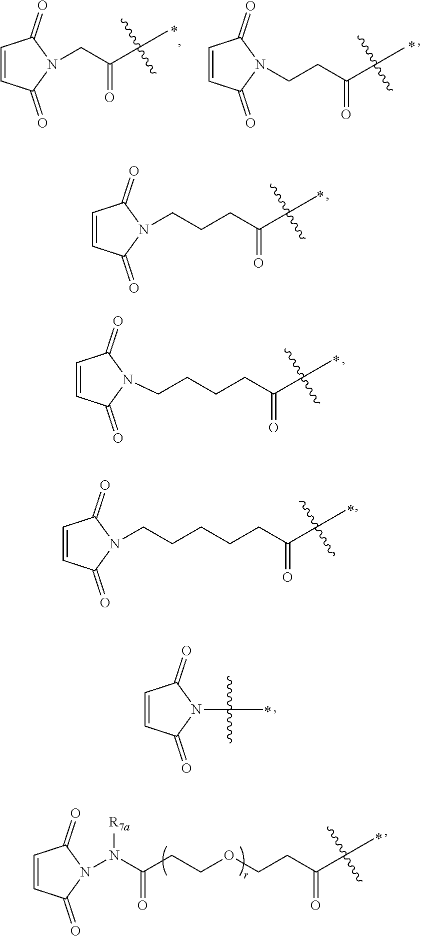 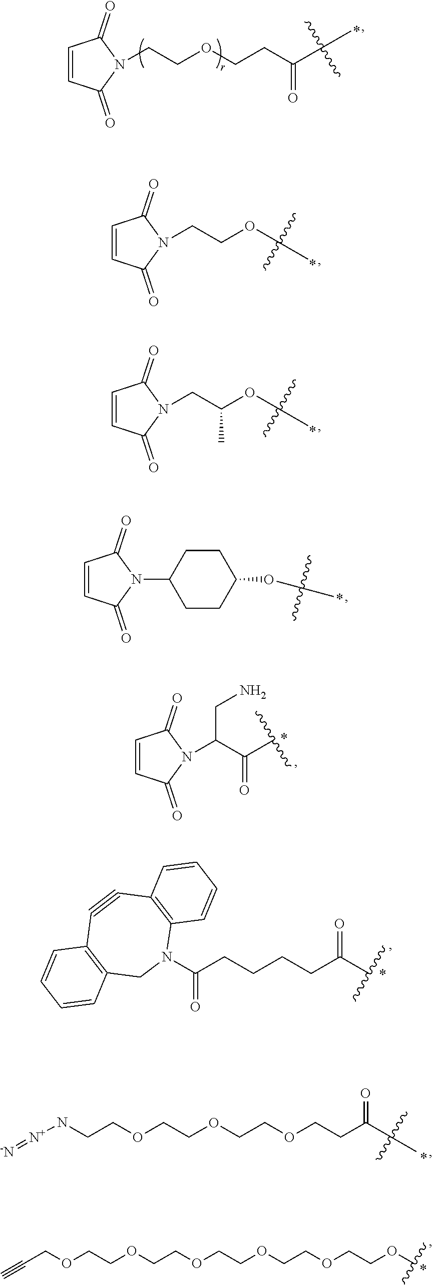
and wherein:
r is an integer ranging from about 4 to about 6; and
# denotes attachment to T and * denotes attachment to L^{C}.

34. The compound-linker construct or conjugate of any one of the preceding claims, wherein H^{1A} is 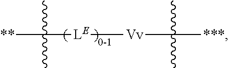
wherein L^{E}, when present, is —NH—[(CH₂CH₂O)p-(CH₂)₀₋₂]q-C(O)—, —NH—(C₁-C₆alkyl)-O—C(O)—, or —NH—[(CH₂CH₂O)p-(CH₂)₀₋₂]q-C(O)—NH—(C₁-C₆alkyl)-O—C(O)—, wherein p is an integer ranging from about 1 to about 20, and q is an integer ranging from about 1 to about 10;
each V independently is a natural or unnatural amino acid, diamine or amino alcohol unit;
v is an integer ranging from about 0 to about 12;
** denotes attachment to M^{A}, when M^{A} is present, or to H^{1B}, when M^{A} is absent;
*** denotes attachment to D.

35. The compound-linker construct or conjugate of any one of the preceding claims, wherein v is 0, 1, 2, 3, 4, or 5.

36. The compound-linker construct or conjugate of any one of the preceding claims, wherein L^{E} is —NH—(CH₂CH₂O)₂—(CH₂)₂—C(O)—, —NH—CH₂—CH(CH₃)—O—C(O)—, or —NH—[(CH₂CH₂O)₁₋₄—(CH₂)₂—C(O)—NH—(CH₂)₂—O—C(O)—.

37. The compound-linker construct or conjugate of any one of the preceding claims, wherein H^{1A} is selected from the group consisting of: 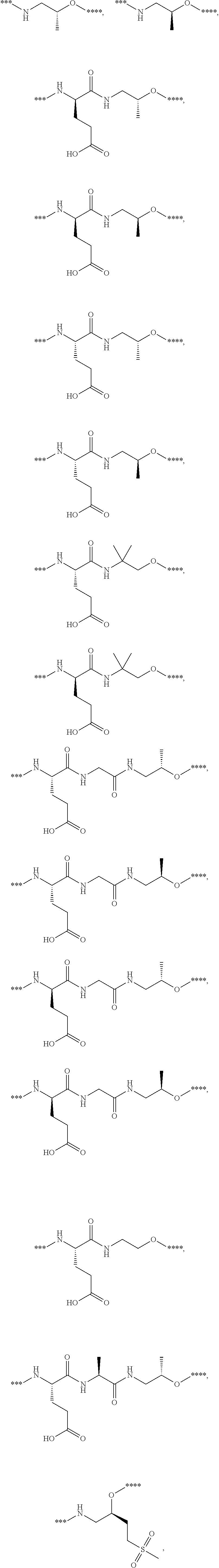 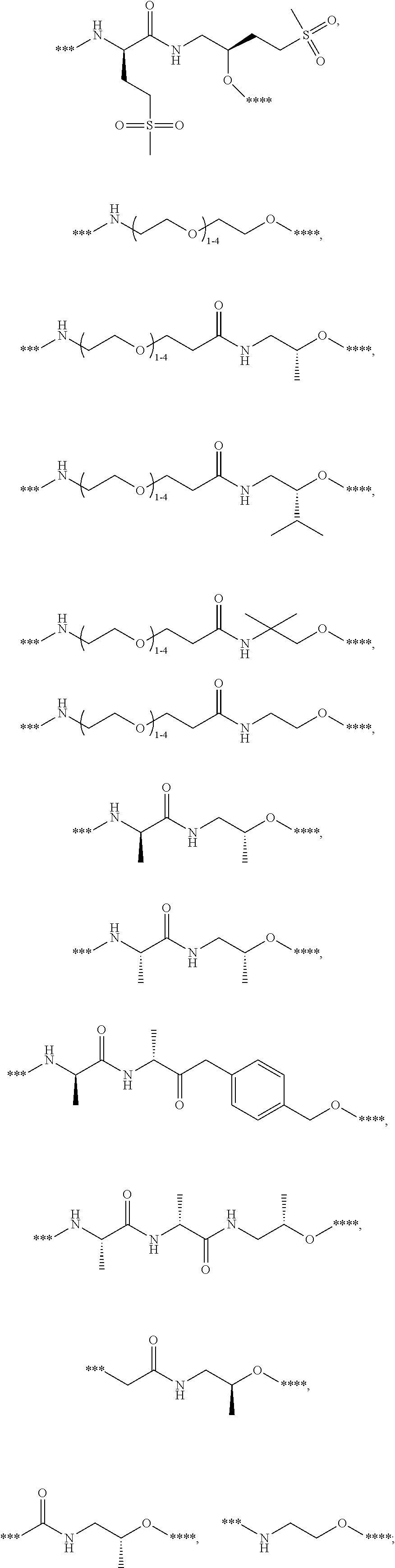
wherein *** denotes attachment to M^{A} when is present, or to H^{1B} when is absent; and ****denotes attachment to D.

38. The compound-linker construct or conjugate of any one of the preceding claims, wherein H^{1A} is selected from the group consisting of: 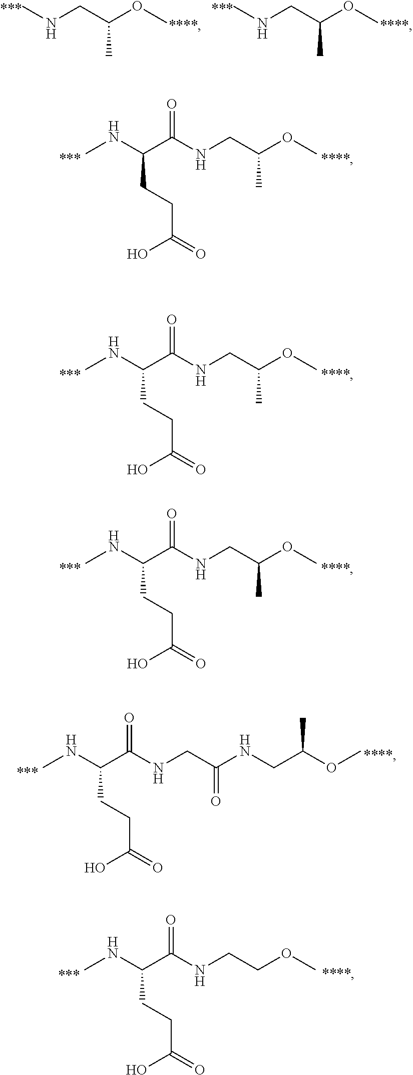 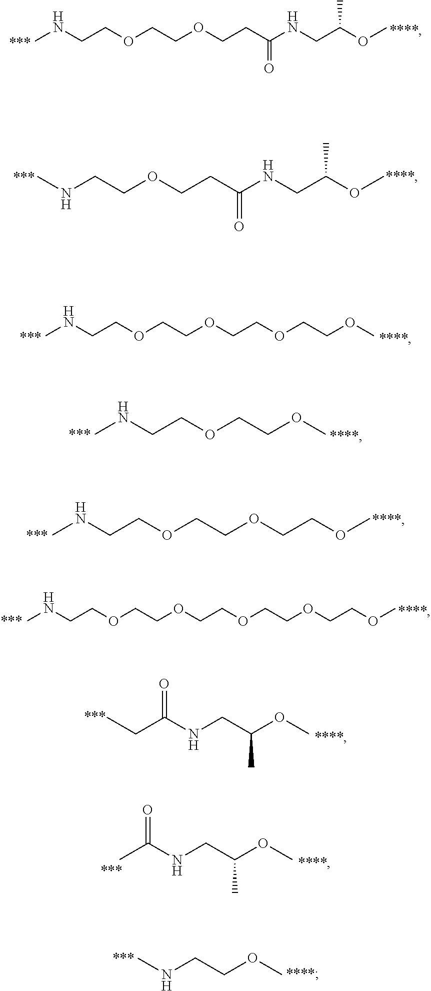
preferably, H^{1A} is selected from the group consisting of: 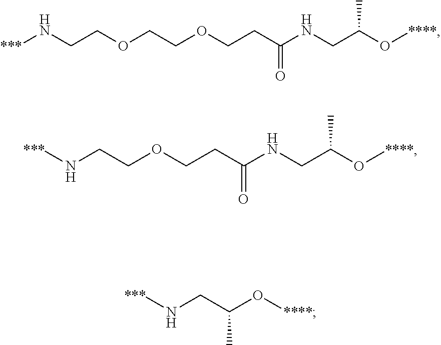
wherein *** denotes attachment to M^{A} when is present, or to H^{1B} when is absent; and ****denotes attachment to D.

39. The compound-linker construct or conjugate of any one of the preceding claims, wherein M^{A} is 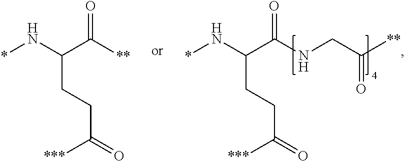
wherein * indicates attachment to H^{1B}; ** indicates attachment to T^{A}; and *** indicates attachment to H^{1A}.

40. The compound-linker construct or conjugate of any one of the preceding claims, wherein T^{A} is 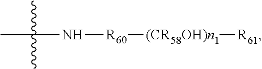
wherein
n₁ is an integer from 0 to about 6;
each R₅₈ is independently —H or C₁₋₈ alkly;
R₆₀ is a bond, a C₁₋₆ alkyl linker, or —CHR₅₉—; wherein R₅₉ is —H, C₁₋₈ alkyl, cycloalkyl, or arylalkyl;
R₆₁ is CH₂OR₆₂, COOR₆₂, —(CH₂)ₙ₂COOR₆₂, or a heterocycloalkyl substituted with one or more hydroxyl;
R₆₂ is —H or C₁₋₈ alkyl; and
n₂ is an integer from 1 to about 5.

41. The compound-linker construct or conjugate of any one of the preceding claims, wherein T^{A} is

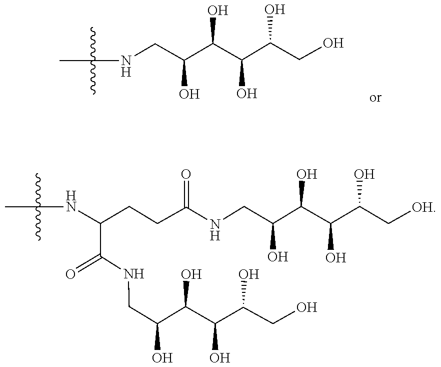 [SMILES: *NC[C@H](O)[C@@H](O)[C@H](O)[C@H](O)CO ; *NC(CCC(=O)NC[C@H](O)[C@@H](O)[C@H](O)[C@H](O)CO)C(=O)NC[C@H](O)[C@@H](O)[C@H](O)[C@H](O)CO]

42. The compound-linker construct or conjugate of any one of the preceding claims, wherein T^{A} is 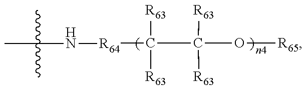
wherein
n4 is an integer from 1 to about 25;
each R₆₃ is independently hydrogen or C₁ s alkyl;
R₆₄ is a bond or a C₁₋₈ alkyl linker;
R₆₅ is H, C₁₋₈ alkyl, —(CH₂)ₙ2COOR₆₂ or —(CH₂)ₙ2COR₆₆;
R₆₂ is H or C₁₋₈ alkyl;
R₆₆ is H, 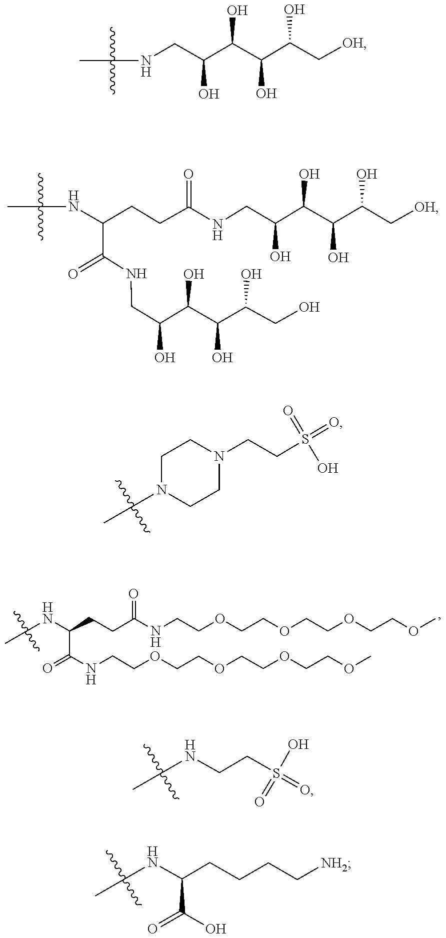 [SMILES: *NC[C@H](O)[C@@H](O)[C@H](O)[C@H](O)CO ; *NC(CCC(=O)NC[C@H](O)[C@@H](O)[C@H](O)[C@H](O)CO)C(=O)NC[C@H](O)[C@@H](O)[C@H](O)[C@H](O)CO ; *N1CCN(CCS(=O)(=O)O)CC1 ; *N[C@@H](CCC(=O)NCCOCCOCCOCCOC)C(=O)NCCOCCOCCOCCOC ; *NCCS(=O)(=O)O ; *N[C@@H](CCCCN)C(=O)O]
and
n2 is an integer from 1 to about 5.

43. The compound-linker construct or conjugate of any one of the preceding claims, wherein T^{A} is 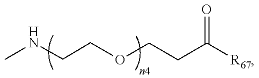
wherein R₆₇ is (1)—OH, 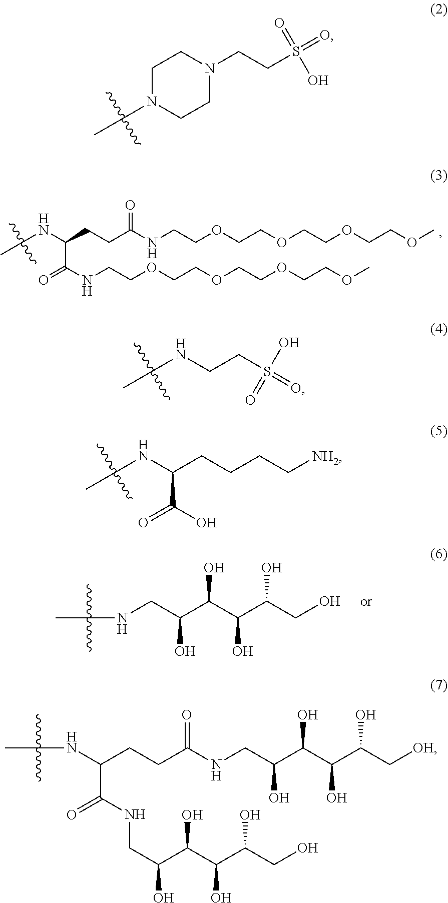
wherein n4 is an integer from about 2 to about 20, from about 4 to about 16, from about 6 to about 12, from about 8 to about 12.

44. The compound-linker construct or conjugate of any one of the preceding claims, wherein n4 is 6, 7, 8, 9, 10, 11, or 12.

45. The compound-linker construct or conjugate of any one of the preceding claims, wherein T^{A} is 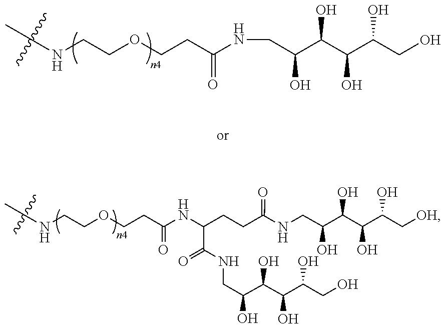
n₄ is an integer from about 2 to about 24, from about 4 to about 16, from about 6 to about 12, from about 8 to about 12.

46. The compound-linker construct or conjugate of any one of the preceding claims, wherein n4 is 6, 7, 8, 9, 10, 11, or 12; preferably 8 or 12; more preferably 8.

47. The compound-linker construct or conjugate of any one of the preceding claims, wherein the linker L¹ has the structure H^{1A}-L^{C}-H^{1B} and is selected from the group consisting of: 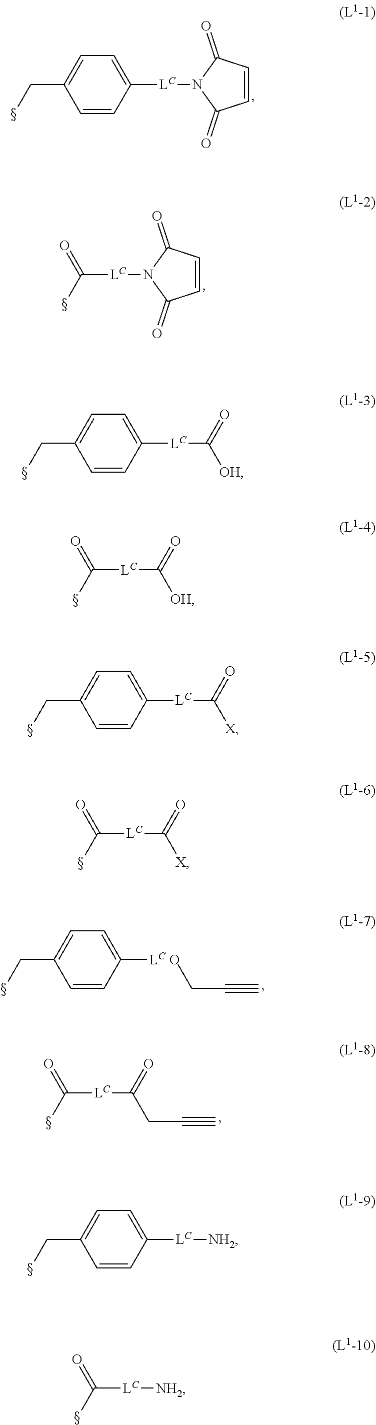 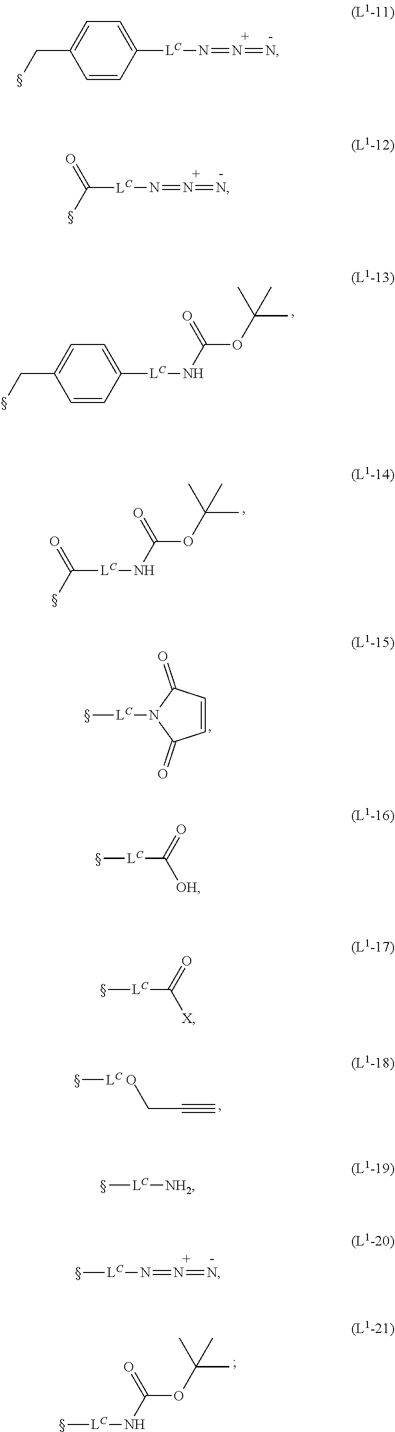
wherein
L^{c} is a chain L^{c} of units selected from a linear or branched polyethylene glycol chain, a sequence of amino acids, and a linear or branched C₁-C₁₀-alkyl chain, wherein each substitutable carbon or heteroatom of the aforementioned units may be unsubstituted or substituted with one or more, same or different substituents selected from halogen, OH, ═O, C₁-C₆-alkyl, C₁-C₆-haloalkyl, C₃-C₆-cycloalkyl, C₃-C₆-halocycloalkyl, C₁-C₆-alkoxy, and C₁-C₆-haloalkoxy; and
§ marks the connection to the compound of formula (A)-(C), I-V; and
X represents a leaving group selected from 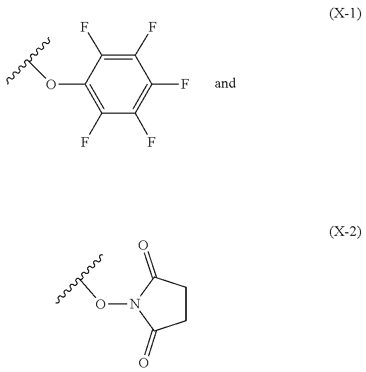 [SMILES: *Oc1c(F)c(F)c(F)c(F)c1F ; *ON1C(=O)CCC1=O]
and wherein preferably L^{c} is selected from the group consisting of 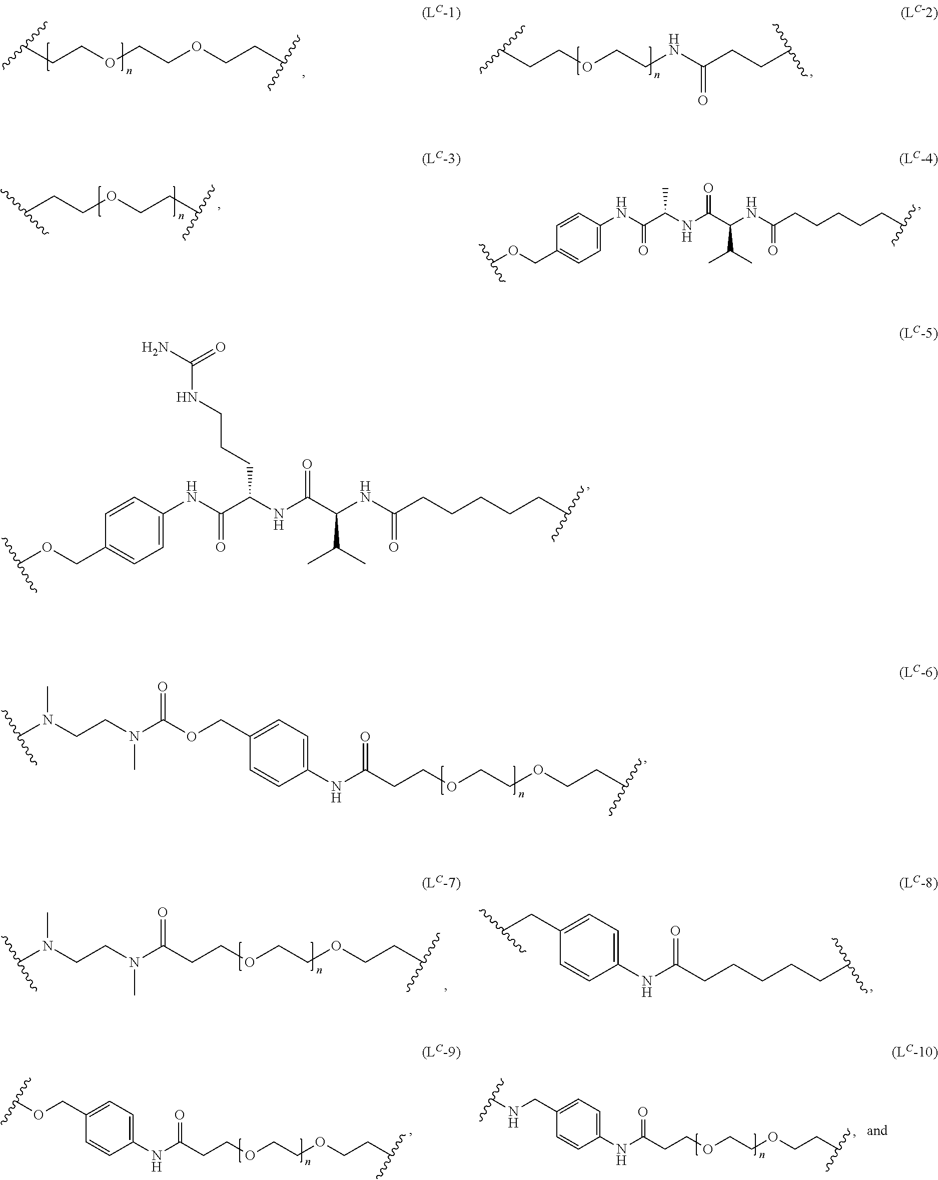 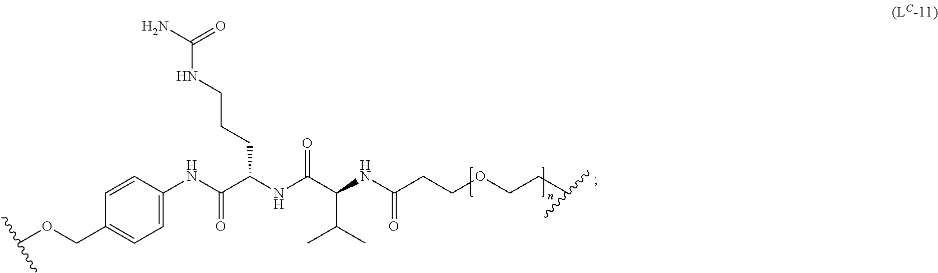
wherein n is 0, 1, 2, 3, 4, 5, 6, 7, or 8.

48. The compound-linker construct or conjugate of any one of the preceding claims, wherein L^{c} is selected from:
—C(═O)(CH₂)ₘ—**; —C(═O)(CH₂)ₘ (CH₂)ₘ—**; —C(═O)XₐX_{b}C(═O)(CH₂)ₘ—**; —C(═O)XₐX_{b} C(═O)(CH₂)ₘO(CH₂)ₘ—**; —C(═O)XₐX_{b}C(═O)(CH₂)ₘO(CH₂)ₘC(═O)—**; —C(═O)X₄C(═O)NH(CH₂)ₘNHC(═O)(CH₂)ₘO(CH₂)ₘ—**; —C(═O)X₅C(═O)(CH₂)ₘNHC(═O)(CH₂)ₘ—**; —C(═O)(CH₂)ₘNHC(═O)XₐX_{b}C(═O)(CH₂)ₘ—**; —C(═O)O(CH₂)ₘNHC(═O)XₐX_{b}C(═O)(CH₂)ₘ—**; —C(═O)(CH₂)ₘNHC(═O)(CH₂)ₘO)ₙ (CH₂)ₘ—**; —C(═O)O(CH₂)ₘNHC(═O)(CH₂)ₘ—**; —C(═O)O(CH₂)ₘNHC(═O)(CH₂)ₘO(CH₂)ₘ—**; —C(═O)O(CH₂)ₘNHC(═O)XₐXC(═O)(CH₂)ₘO(CH₂)ₘ—**; —X₂C(═O)(CH₂)ₘ—**-; —C(═O)X₅C(═O)(CH₂)ₘNHC(═O)(CH₂)ₘ—**; —C(═O)O(CH₂)ₘNHC(═O)XₐX_{b} C(═O)(CH₂)ₘO(CH₂)ₘC(═O)—**; —C(═O)O(CH₂)ₘNHC(═O)X₄C(═O)NH(CH₂)ₘNHC(═O)(CH₂)ₘO(CH₂)ₙ—**; —C(═O)O((CH₂)ₘO)ₙ(CH₂)ₘNHC(═O)X₅C(═O)(CH₂)ₘ—**; —C(═O)O((CH₂)ₘO)ₙ(CH₂)ₘNHC(═O)X₅C(═O)(CH₂)ₘNHC(═O)(CH₂)ₘ—**; —C(═O)O((CH₂)ₘO)ₙ(CH₂)ₘNHC(═O)X₅C(═O)(CH₂)ₘX_{c}(CH₂)ₘ—**; —C(═O)O((CH₂)ₘO)ₙ(CH₂)ₘNHC(═O)X₅C(═O)(CH₂)ₘO)ₙ (CH₂)ₘ—**; —C(═O)O((CH₂)ₘO)ₙ(CH₂)ₘNHC(═O)X₅ C(═O)((CH₂)ₘO)ₙ(CH₂)ₘNHC(═O)(CH₂))ₘ—**; —C(═O)O(CH₂)ₘO)ₙ (CH₂)ₘNHC(═O)X₅ C(═O)(CH₂)ₘO)ₙ(CH₂)ₘNHC(═O)(CH₂)ₘX_{c}(CH₂)ₘ—**; —C(═O)O((CH₂)ₘO)ₙ(CH₂)ₘNHC(═O))X₅C(═O)(CH₂)ₘO)ₙ (CH₂)ₘX_{c}(CH₂)ₘ—**; —C(═O)O((CH₂)ₘO)ₙ(CH₂)ₘNHC(═O)X₅C(═O)(CH₂)ₘNHC(═O)((CH₂)ₘO)ₙ(CH₂)ₘ—**; —C(═O)O((CH₂)ₘO)ₙ(CH₂)ₘNHC(═O)X₅C(═O)(CH₂)ₘNHC(═O)(CH₂)ₘO)ₙ (CH₂)ₘX_{c}(CH₂)ₘ—**; —C(═O)O((CH₂)ₘO)ₙ(CH₂)ₘNHC(═O)X₅(CH₂)ₘX_{c}(CH₂)ₘ—**; —C(═O)O((CH₂)ₘO)ₙ(CH₂)ₘNHC(═O)X₅((CH₂)ₘO)ₙ(CH₂)ₘ—**; —C(═O)O((CH₂)ₘO)ₙ(CH₂)ₘNHC(═O)X₅(CH₂)ₘO)ₙ (CH₂)ₘNHC(═O)(CH₂)ₘ—**; —C(═O)O((CH₂)ₘO)ₙ(CH₂)ₘNHC(═O)X₅(CH₂)ₘO)ₙ (CH₂)ₘNHC(═O)(CH₂)ₘX_{c}(CH₂)ₘ—**; —C(═O)O((CH₂)ₘO)ₙ(CH₂)ₘNHC(═O)X₅(CH₂)ₘO)ₙ (CH₂)ₘX_{c}(CH₂)ₘ—**; —C(═O)O((CH₂)ₘO)ₙ(CH₂)ₘNHC(═O)X₅(CH₂)ₘNH(CH₂)ₘO)ₙ (CH₂)ₘ—**; —C(═O)O((CH₂)ₘO)ₙ(CH₂)ₘNHC(═O)X₅C(═O)(CH₂)ₘNH(CH₂)ₘO)ₙ (CH₂)ₘX_{c}(CH₂)ₘ—**; —C(═O)O(CH₂)ₘO)ₙ (CH₂)ₘNHC(═O)X₅(CH₂)ₘ—**; —C(═O)O(CH₂)ₘ—**; —C(═O)O((CH₂)ₘO)ₙ(CH₂)ₘ—**; —C(═O)O(CH₂)ₘNH(CH₂)ₘ—**; —C(═O)O(CH₂)ₘNH(CH₂)ₘC(═O)XₐXC(═O)—**; —C(═O)O(CH₂)ₘX_{c}(CH₂)ₘ—**;
—C(═O)O(CH₂)ₘNHC(═O)XₐX_{b} C(═O)CH₂)ₘO)ₙ (CH₂)ₘC(═O)—**; —C(=)O((CH₂)ₘO)ₙ(CH₂)ₘX_{c}(CH₂)ₘ—**; —C(═O)O((CH₂)ₘO)ₙ(CH₂)ₘNHC(═O)(CH₂)ₘ—**; —C(═O)O(CH₂)ₘNHC(═O(CH₂)ₘX_{c}(CH₂)ₘ—**; —C(═O)O((CH₂)ₘO)ₙ(CH₂)ₘNHC(═O)(CH₂) mXc(CH₂)ₘ—**; —C(═O)O((CH₂)ₘO)ₙX₃(CH₂)ₘ—**; —C(═O)O((CH₂)ₘO)ₙ(CH₂)ₘX₃(CH₂)ₘ—**; —C(═O)O((CH₂)ₘO)ₙ(CH₂)ₘC(═O)NH(CH₂)ₘ—**; —C(═O)O(CH₂)ₘC(R¹²)₂—**; —C(═O)O(CH₂)ₘSSC(R¹²)₂(CH₂)ₘC(═O)NR¹²(CH₂)ₘNR¹²C(═O)(CH₂)ₘO(CH₂)ₘ—**; —C(═O)O(CH₂)ₘSSC(R¹²)₂(CH₂)ₘC(═O)NR¹²(CH₂)ₘNR¹²C(═O)(CH₂)ₘ—**; —C(═O)O(CH₂)ₘC(═O)NH(CH₂)ₘ—**; —C(═O)((CH₂)ₘO)ₙ(CH₂)ₘ—**; —C(═O)(CH₂)ₘNH(CH₂)ₘ—**; —C(═O)(CH₂)ₘNH(CH₂)ₘC(═O)XₐX_{b} C(═O)—**; —C(═O)(CH₂)ₘX_{c}(CH₂)ₘ—**; —C(═O)((CH₂)ₘO)ₙ(CH₂)ₘX_{c}(CH₂)ₘ—**; —C(═O)(CH₂)ₘNHC(═O)(CH₂)ₘ—**; —C(═O)(CH₂)ₘO)ₙ (CH₂)ₘNHC(═O)(CH₂)ₘ—**; —C(═O)(CH₂)ₘNHC(═O(CH₂)ₘX_{c}(CH₂)ₘ—**; —(CH₂)ₘNHC(═O)XₐX_{b} C(═O)(CH₂)ₘ—**; —(CH₂)ₘ(CHOH)(CH₂)ₘNHC(═O)XₐX_{b} C(═O)(CH₂)ₘ**; —C(═O)((CH₂)ₘO)ₙ(CH₂)ₘNHC(═O)(CH₂)ₘX_{c}(CH₂)ₘ—**; —C(═O)((CH₂)ₘO)nXc(CH₂)ₘ—**; —C(═O)((CH₂)ₘO)ₙ(CH₂)ₘC(═O)NH(CH₂)ₘ—**; —C(═O)(CH₂)ₘC(R¹²)₂—**: —C(═O)((CH₂)ₘO)ₙ(CH₂)ₘNHC(═O)X₅ C(═O)(CH₂)ₘ—**; —C(═O)((CH₂)ₘO)ₙ(CH₂)ₘNH C(═O)X₅ C(═O)(CH₂)ₘNHC(═O)(CH₂)ₘ—**; —C(═O)((CH₂)ₘO)ₙ(CH₂)ₘNH C(═O)X₅ C(═O)(CH₂)ₘX_{c}(CH₂)ₘ—**; —C(═O)((CH₂)ₘO)ₙ(CH₂)ₘNH C(═O)X₅C(═O)((CH₂)ₘO)ₙ(CH₂)ₘ—**; —C(═O)((CH₂)ₘO)ₙ(CH₂)ₘNHC(═O)X₅C(═O)((CH₂)ₘO)ₙ(CH₂)ₘNHC(═O)(CH₂)ₘ—**; —C(═O)((CH₂)ₘO)(CH₂)ₘNHC(═O)X₅C(═O)((CH₂)ₘO)ₙ(CH₂)ₘNHC(═O)(CH₂)ₘX_{c}(CH₂)ₘ—**; —C(═O)((CH₂)ₘO)(CH₂)ₘNHC(═O))X₅C(═O)(CH₂)ₘO)ₙ (CH₂)ₘX_{c}(CH₂)ₘ—**; —C(═O)(CH₂)ₘO)ₙ (CH₂)ₘNHC(═O)X₅ C(═O)(CH₂)ₘNHC(═O)(CH₂)ₘO)ₙ (CH₂)ₘ—**; —C(═O)((CH₂)ₘO)ₙ(CH₂)ₘNHC(═O)X₅C(═O)(CH₂)ₘNHC(═O)((CH₂)ₘO)ₙ(CH₂)ₘX_{c}(CH₂))ₘ—**; —C(═O)((CH₂)ₘO)ₙ(CH₂)ₘNHC(═O)X₅(CH₂)ₘX_{c}(CH₂)ₘ—**; —C(═O)(CH₂)ₘO)ₙ (CH₂)ₘNHC(═O)X₅((CH₂)ₘO)ₙ(CH₂)ₘ—**; —C(═O)(CH₂)ₘO)ₙ (CH₂)ₘNHC(═O)X₅(CH₂)ₘO)ₙ (CH₂)ₘNHC(═O)(CH₂)ₘ—**; —C(═O)((CH₂)ₘO)ₙ(CH₂)ₘNHC(═O)X((CH₂)ₘO)ₙ(CH₂)ₘNHC(═O)(CH₂)ₘX_{c}(CH₂)ₘ—**; —C(═O)(CH₂)ₘO)ₙ (CH₂)ₘNHC(═O)X₅(CH₂)ₘO)ₙ (CH₂)ₘX_{c}(CH₂)ₘ—**; —C(═O)((CH₂)ₘO)ₙ(CH₂)ₘNHC(═O)X₅(CH₂)ₘNH((CH₂)ₘO)ₙ(CH₂)ₘ—**; —C(═O)((CH₂)ₘO)ₙ(CH₂)ₘNHC(═O)X₅C(═O)(CH₂)ₘNH((CH₂)ₘO)ₙ(CH₂)ₘX_{c}(CH₂)ₘ—**; —C(═O)((CH₂)ₘO)ₙ(CH₂)ₘNHC(═O)X₅(CH₂)ₘ—**; —C(═O)(CH₂)ₘC(═O)NH(CH₂)ₘ—**; —C(═O)XₐX_{b} C(═O)(CH₂)ₘNHC(═O)(CH₂)ₘ—**; —C(═O)XₐX_{b}O(═O)(CH₂)ₘX_{c}(CH₂)ₘ—**; —C(═O)XₐX_{b}C(═O)(CH₂)ₘO)ₙ(CH₂)ₘ—**; —C(═O)XₐX_{b}C(═O)((CH₂)ₘO)ₙ(CH₂)ₘNHC(═O)(CH₂)ₘ—**; —C(═O)XₐX_{b} C(═O)(CH₂)ₘO)ₙ(CH₂)ₘNHC(═O)(CH₂)ₘX_{c}(CH₂)ₘ—**; —C(═O)XₐX_{b} C(═O)(CH₂)ₘO)ₙ (CH₂)ₘX_{c}(CH₂)ₘ—**; —C(═O)XₐX_{b} C(═O)(CH₂)ₘNHC(═O)((CH₂)ₘO)ₙ(CH₂)ₘ—**; —C(═O)XₐX_{b}C(═O)(CH₂)ₘ,NHC(═O)(CH₂)ₘO)ₙ(CH₂)ₘX_{c}(CH₂)ₘ—**; —C(═O)XₐX_{b}(CH₂)ₘX_{c}(CH₂)ₘ—**; C(═O)XₐX_{b} ((CH₂)ₘO) n (CH₂) m-**; —C(═O)XₐX_{b}((CH₂)ₘO)ₙ(CH₂)ₘNHC(═O)(CH₂))ₘ—**; —C(═O)XₐX_{b}(CH₂)ₘO)ₙ(CH₂)ₘNHC(═O)(CH₂)ₘX_{c}(CH₂)ₘ—**; —C(═O)XₐX_{b}(CH₂)ₘO)ₙ (CH₂)ₘX_{c}(CH₂)ₘ—**; —C(═O)XₐX_{b}(CH₂)ₘNH((CH₂)ₘO)ₙ(CH₂)ₘ—**; —C(═O)XₐX_{b} C(═O)(CH₂)ₘNH((CH₂)ₘO)ₙ(CH₂)mXC(CH₂)ₘ—**; —C(═O)XₐC(═O)NH(CH₂)ₘX₅(CH₂)ₘ—**; —C(═O)XₐX_{b}(CH₂)ₘ—**; —C(═O)XC(═O)(CH₂)ₘNHC(═O)((CH₂)ₘO)(CH₂)ₘ—**; —C(═O)NH(CH₂)ₘ—**; —C(═O)NH(CH₂)ₘX_{c}(CH₂)ₘ**; —C(═O)NH(CH₂)ₘNHC(═O)XₐX_{b} C(═O)(CH₂)ₘ—**; —C(═O)NH(CH₂)ₘNHC(═O)Q(CH₂)ₘ—**; —C(═O)NH(CH₂)ₘNHC(═O)XₐX_{b}—**; —C(═O)NH(CH₂)ₘNHC(═O)X₅—**; —C(═O)NH(CH₂)ₘNHC(═O)(CH₂)ₘX₅(CH₂)ₘ—**; —C(═O)NHC(═O)(CH₂)ₘ NHC(═O)XₐX_{b} C(═O)(CH₂)ₘ—**; C(═O)NH(CH₂)ₘNHC(═O)(CH₂)ₘ—**; —C(═O)NH(CH₂)ₘNHC(═O)(CH₂)ₘO(CH₂)ₘ—**; —C(═O)NH(CH₂)ₘNHC(═O)XₐX_{b} C(═O)(CH₂)ₘO(CH₂)ₘ—**; —C(═O)NH(CH₂)ₘNHC(═O)XₐX_{b} C(═O)(CH₂)ₘO(CH₂)ₘC(═O)—**; —C(═O)NH(CH₂)ₘNHC(═O)X₄C(═O)NH(CH₂)ₘNHC(═O)(CH₂)ₘO(CH₂)ₘ—**: —C(═O)NH(CH₂)ₘNHC(═O)X₅C(═O)(CH₂)ₘ—**; —C(═O)NH(CH₂)ₘNH C(═O)X₅ C(═O)(CH₂)ₘNHC(═O)(CH₂)ₘ—**; —C(═O)NH(CH₂)ₘNHC(═O)X₅C(═O)(CH₂)ₘX_{c}(CH₂)ₘ—**; —C(═O)NH(CH₂)ₘNH C(═O)X₅C(═O)(CH₂)ₘO)ₙ (CH₂)ₘ—**; —C(═O)NH(CH₂)ₘNH C(═O)X₅ C(═O)(CH₂)ₘO)ₙ (CH₂)ₘNHC(═O)(CH₂)ₘ—**; —C(═O)NH(CH₂)ₘNH C(═O)X₅ C(═O)(CH₂)ₘO)ₙ (CH₂)ₘNHC(═O)(CH₂)ₘX_{c}(CH₂)ₘ—**; —C(═O)NH(CH₂)ₘNHC(═O)X₅C(═O)((CH₂)ₘO)ₙ(CH₂)ₘX_{c}(CH₂)ₘ—**; - C(═O)NH(CH₂)ₘNHC(═O)X₅C(═O)(CH₂)ₘNHC(═O)(CH₂)ₘO)ₙ (CH₂)ₘ—**; —C(═O)NH(CH₂)ₘNH C(═O)X₅ C(═O)(CH₂)ₘNHC(═O)(CH₂)ₘO)ₙ (CH₂)ₘX_{c}(CH₂)ₘ—**; —C(═O)NH(CH₂)ₘNHC(═O)X₅(CH₂)mXC(CH₂)ₘ—**; —C(═O)NH(CH₂)ₘNHC(═O)X₅((CH₂)ₘO)ₙ(CH₂)ₘ—**; —C(═O)NH(CH₂)ₘNHC(═O)X₅(CH₂)ₘO)ₙ (CH₂)ₘNHC(═O)(CH₂)ₘ—**; —C(═O)NH(CH₂)ₘNHC(═O)X₅((CH₂)ₘO)ₙ(CH₂)ₘNHC(═O)(CH₂)ₘX_{c}(CH₂)ₘ—**; - C(═O)NH(CH₂)ₘNHC(═O)X₅((CH₂)ₘO)ₙ(CH₂)ₘX_{c}(CH₂)ₘ—**; —C(═O)NH(CH₂)ₘNHC(═O)X₅(CH₂)ₘNH(CH₂)ₘO)ₙ (CH₂)ₘ—**; —C(═O)NH(CH₂)ₘNH C(═O)X₅ C(═O)(CH₂)ₘNH((CH₂)ₘO)ₙ(CH₂)ₘX_{c}(CH₂)ₘ—**; —C(═O)NH(CH₂)ₘNHC(═O)X₅(CH₂)ₘ—**; —C(═O)X₁C(═O)NH(CH₂)ₘNHC(═O)(CH₂)ₘ—**; —C(═O)X₁C(═O)NH(CH₂)ₘX_{c}(CH₂)ₘ—**; —C(═O)NH(CH₂)ₘNHC(═O)(CH₂)ₘX_{c}(CH₂)ₘ—**; —C(═O)NH(CH₂)ₘNHC(═O)—**; —C(═O)NH((CH₂)ₘO)ₙ(CH₂)ₘX_{c}(CH₂)ₘ—** or —C(═O)X₁C(═O)(CH₂)ₘNHC(═O)(CH₂)ₘ—**;
Xₐ is 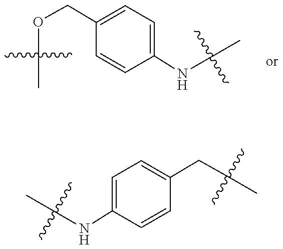 [SMILES: *Nc1ccc(CO*)cc1 ; *Cc1ccc(N*)cc1]
where the * of Xₐ indicates the point of attachment to X_{b};
X_{b} is selected from 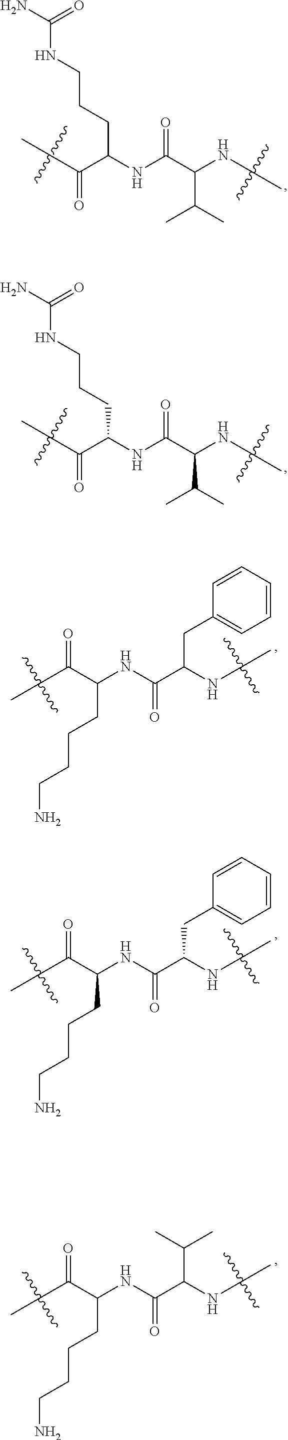 [SMILES: *NC(C(=O)NC(CCCNC(N)=O)C(*)=O)C(C)C ; *N[C@H](C(=O)N[C@@H](CCCNC(N)=O)C(*)=O)C(C)C ; *NC(Cc1ccccc1)C(=O)NC(CCCCN)C(*)=O ; *N[C@@H](Cc1ccccc1)C(=O)N[C@@H](CCCCN)C(*)=O ; *NC(C(=O)NC(CCCCN)C(*)=O)C(C)C] 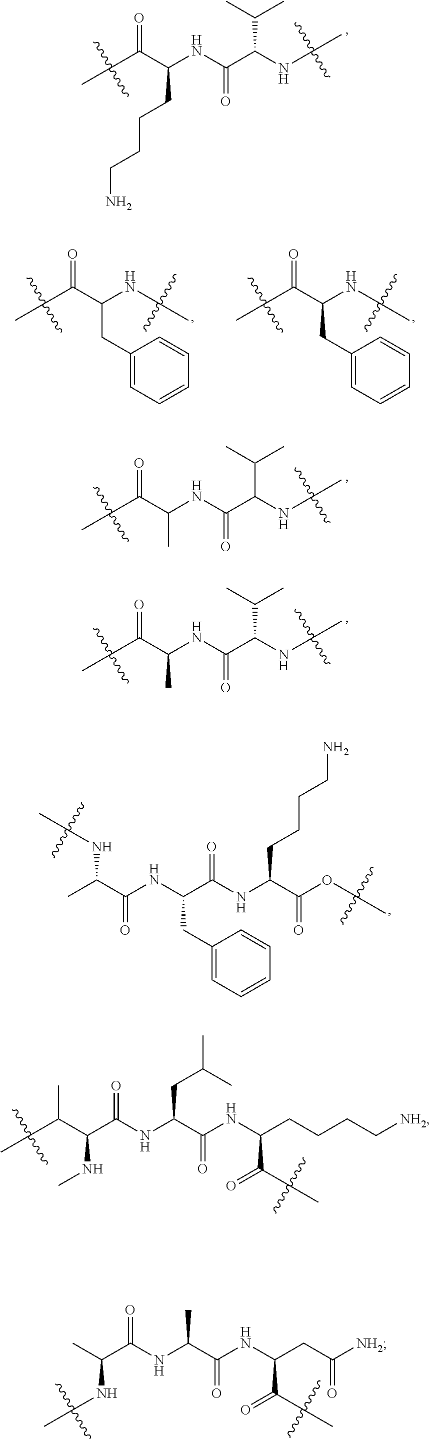
where the * of X_{b} indicates the point of attachment to Xₐ, —NH—, NHNH—, —NHO— or —NHN═CR¹²(CH₂)ₙ—;
X_{c} is 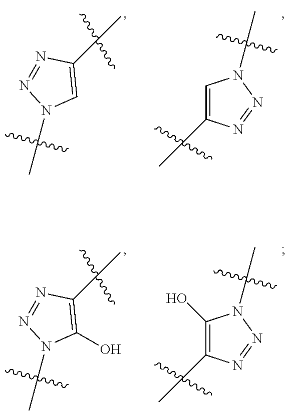 [SMILES: *c1cn(*)nn1 ; *c1cn(*)nn1 ; *c1nnn(*)c1O ; *c1nnn(*)c1O]
X₄ is —O(CH₂)ₙSSC(R¹²)₂(CH₂)ₙ—or —(CH₂)ₙC(R¹²)₂SS(CH₂)ₙO—; 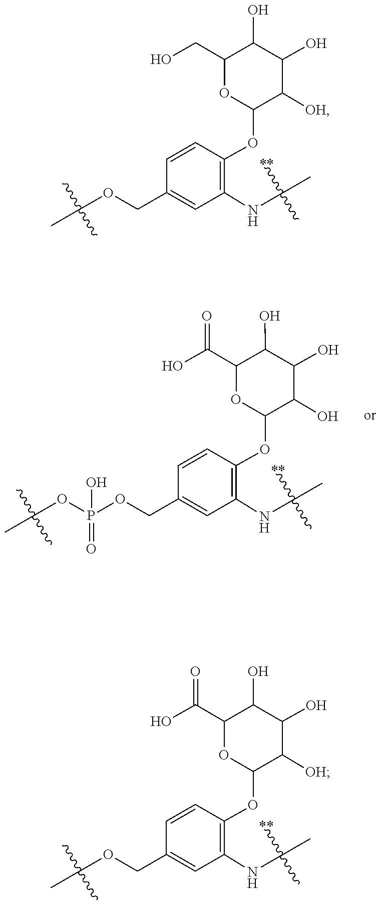 [SMILES: *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]
X₅ is
each m is independently selected from 1, 2, 3, 4, 5, 6, 7, 8, 9 or 10; and
each n is independently selected from 1, 2, 3, 4, 5, 6, 7, 8, 9, 10, 11, 12, 13, 14, 15, 16, 17 or 18.

49. The compound-linker construct or conjugate of any one of the preceding claims, wherein the linker L¹ is selected from:

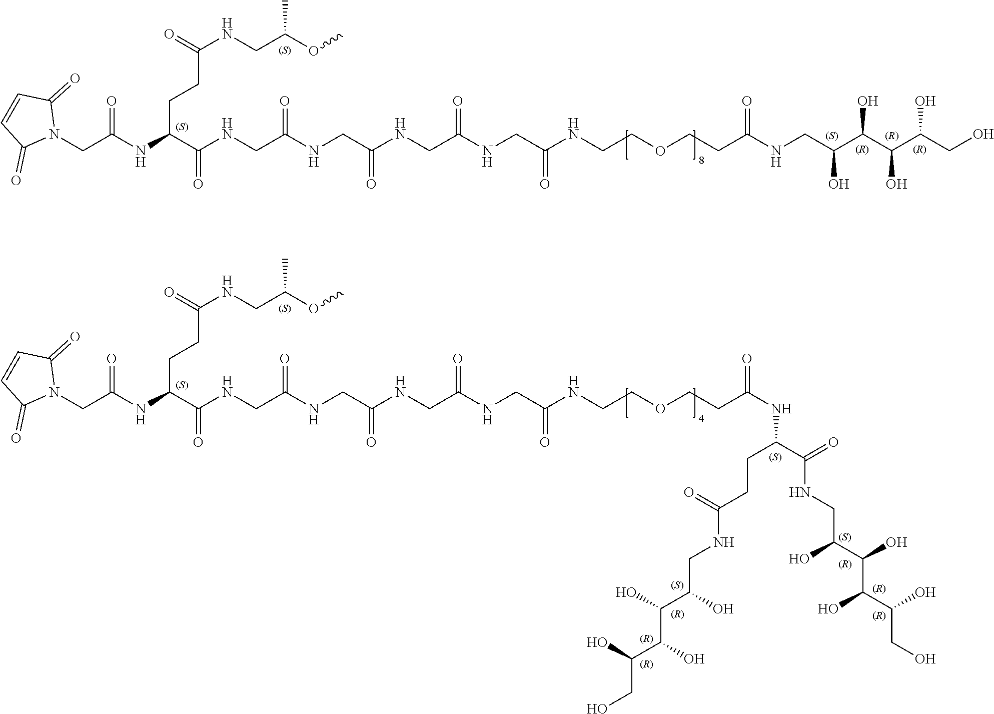

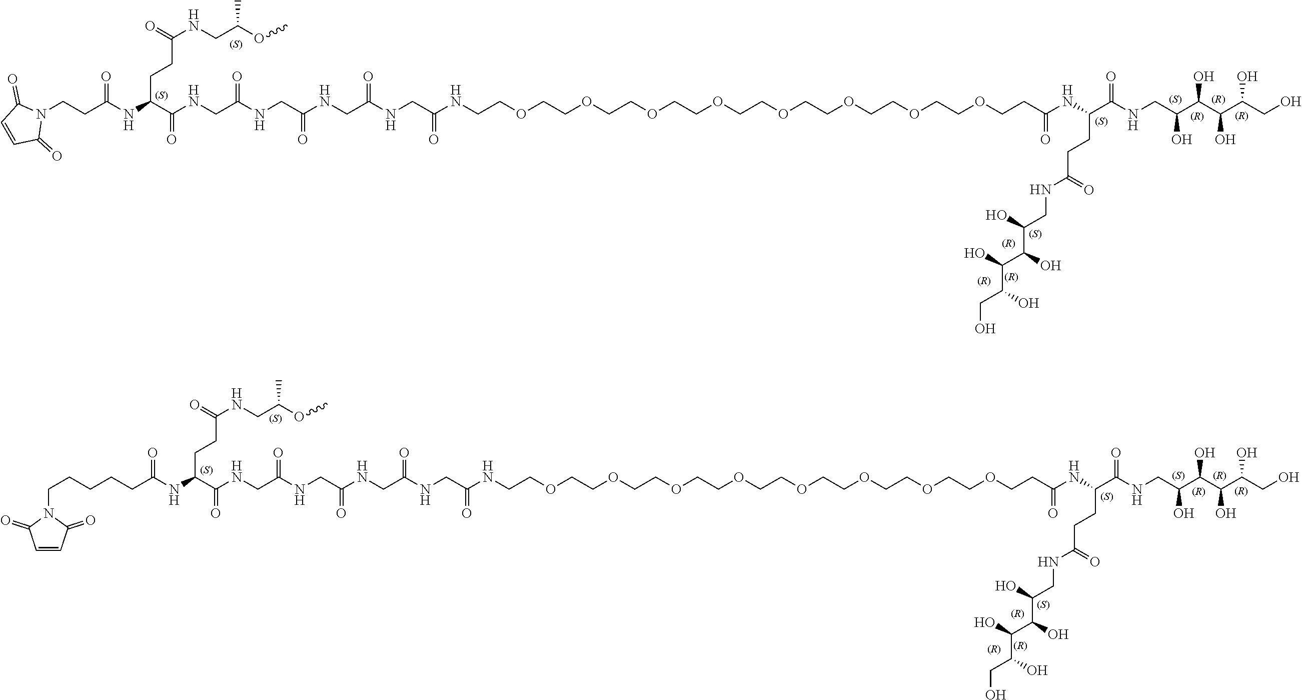

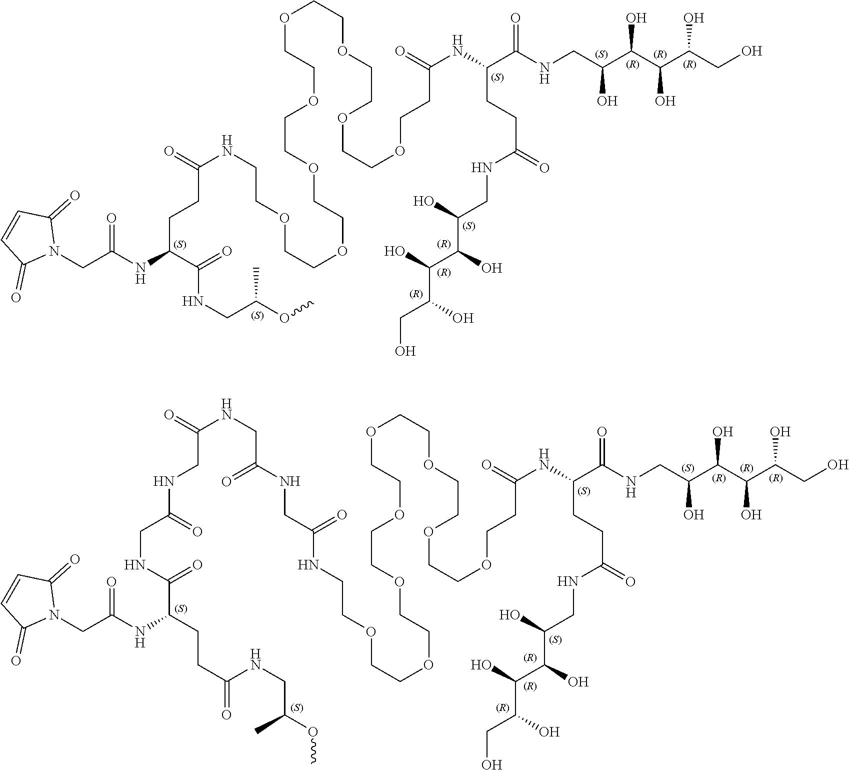

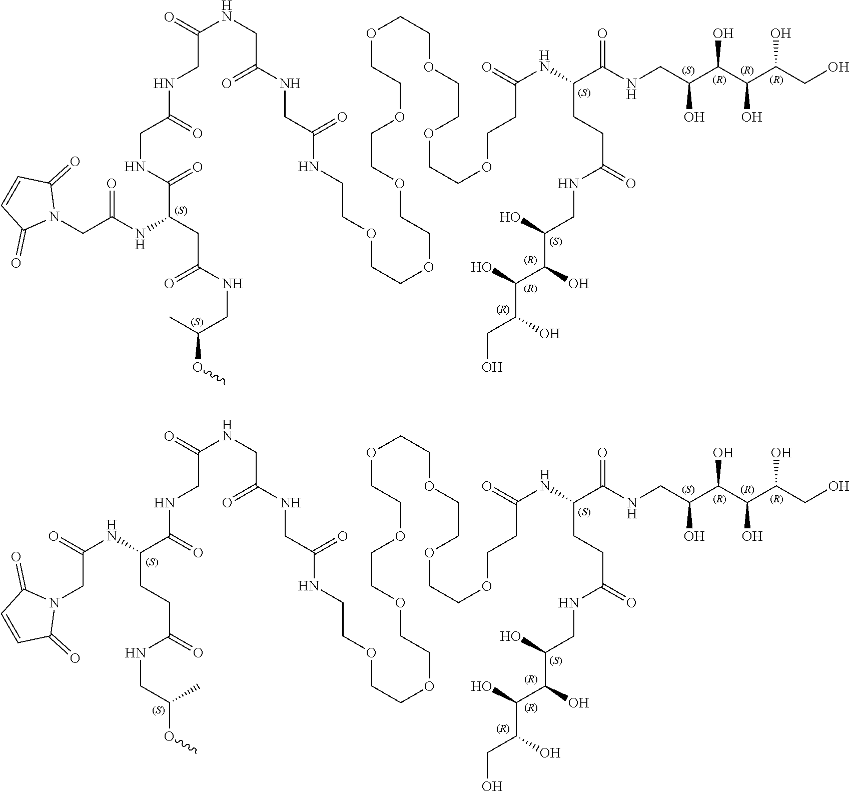

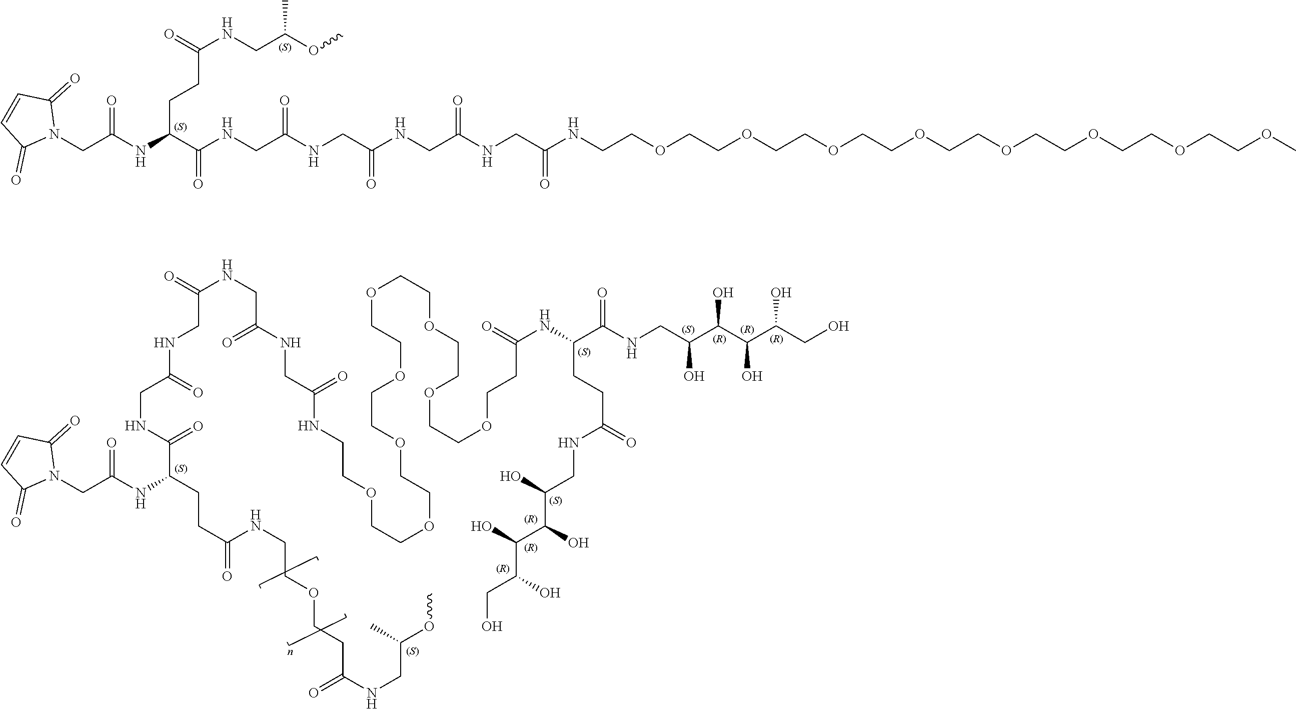

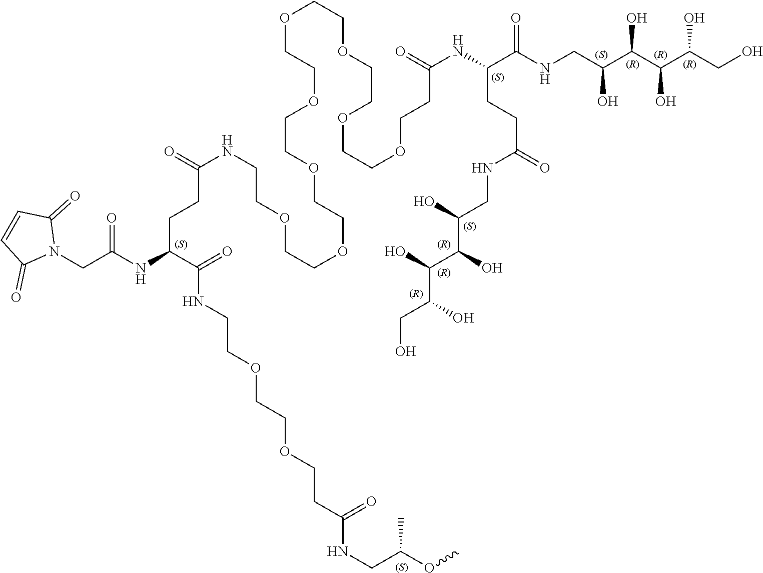

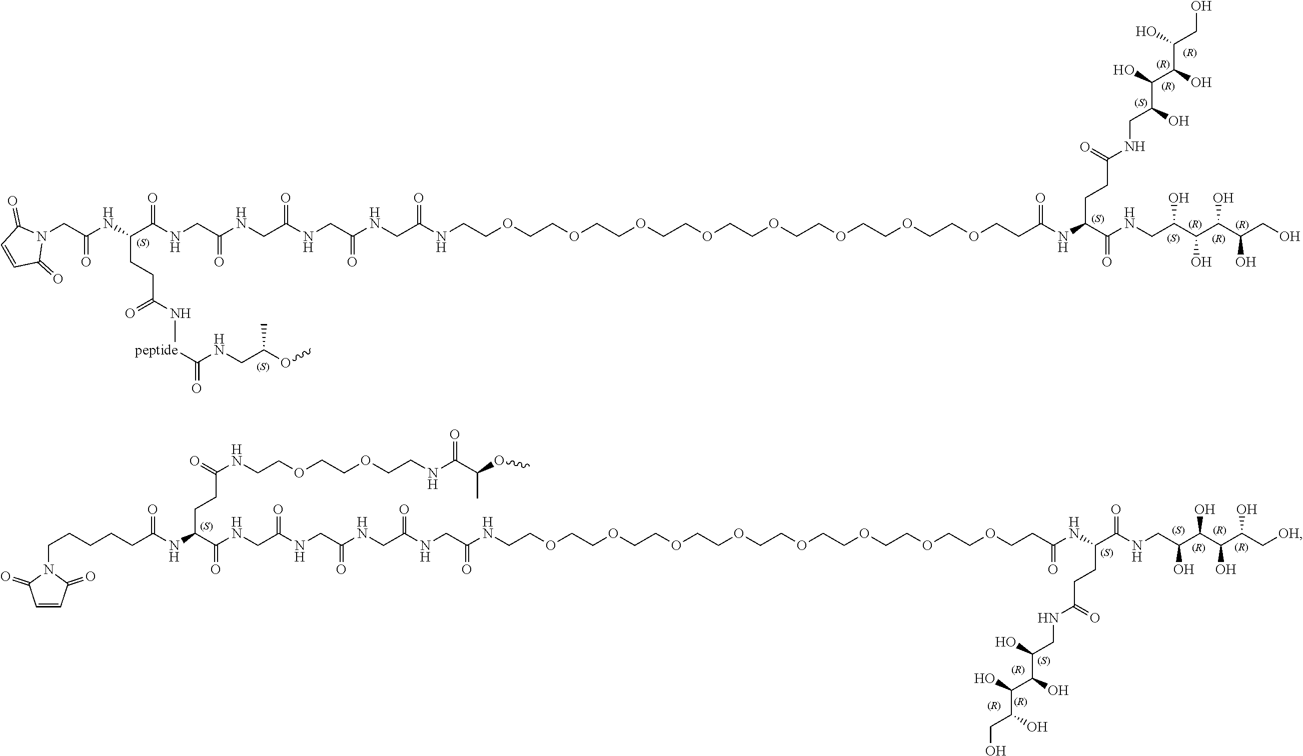

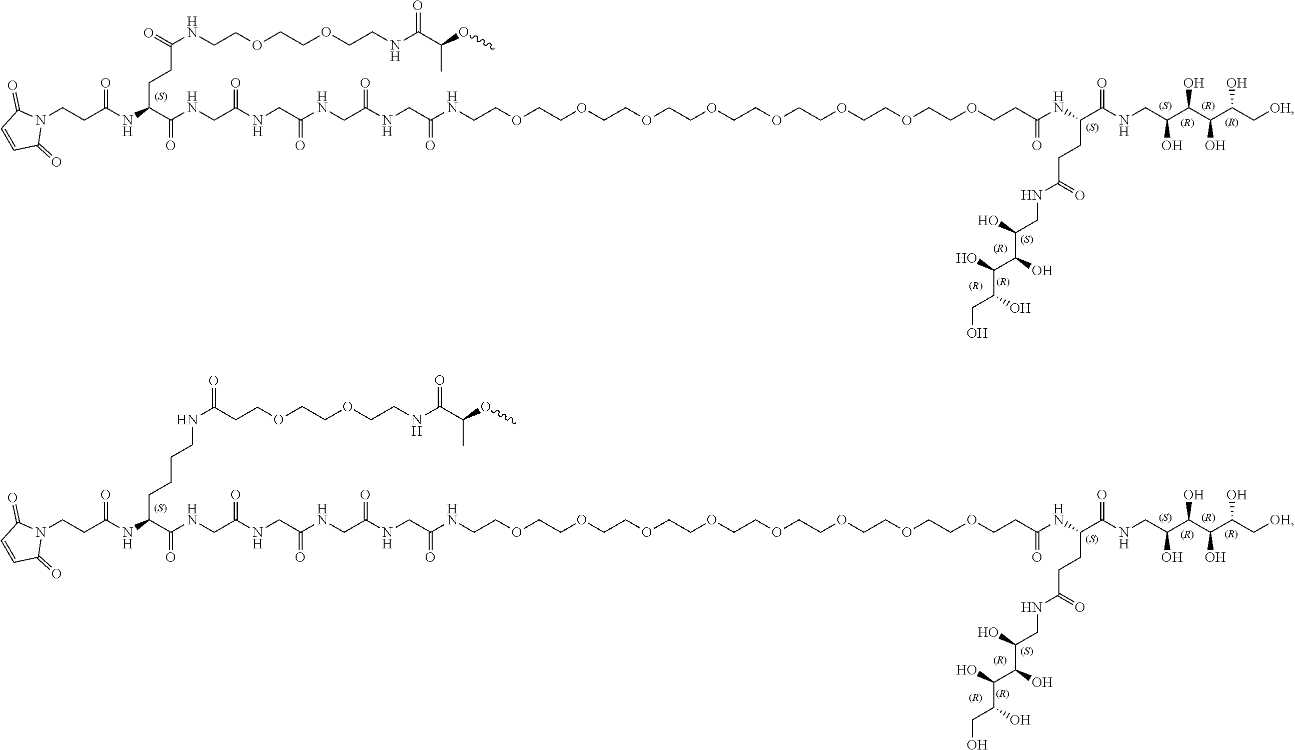

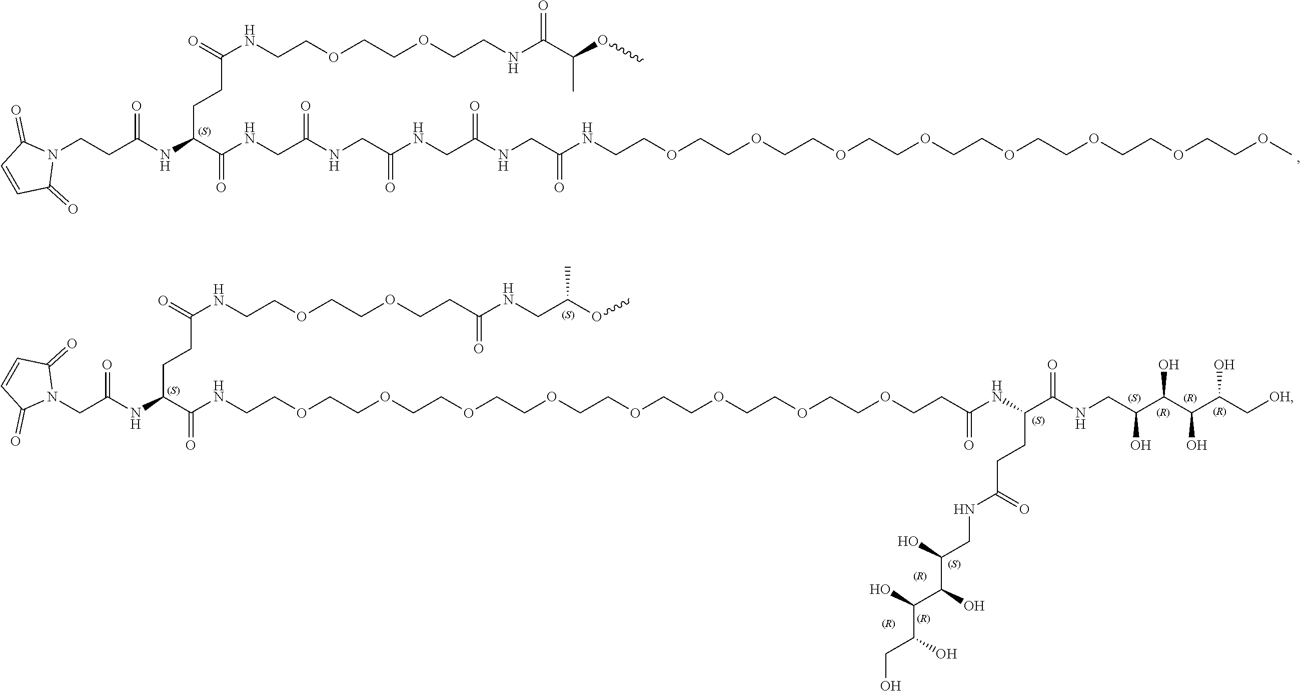

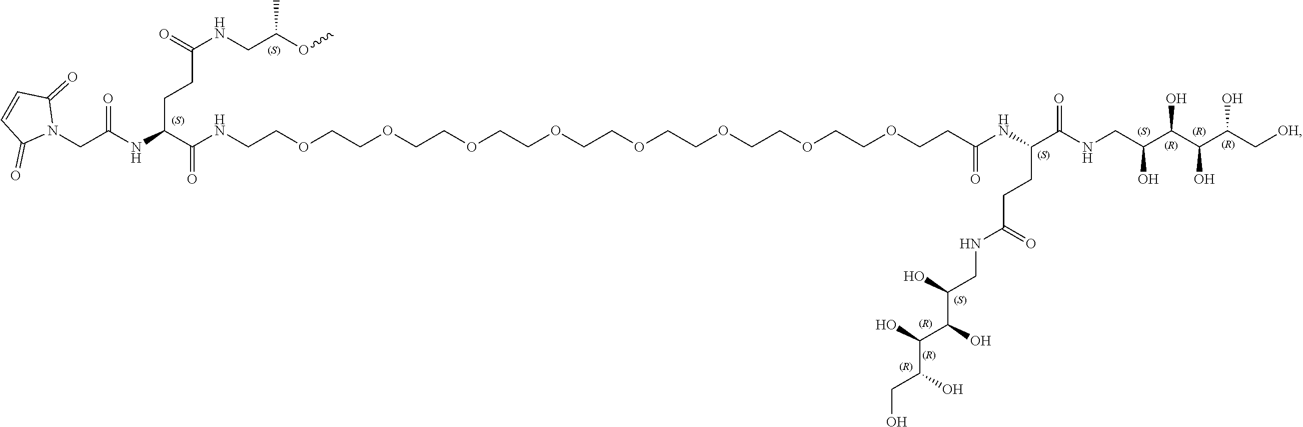

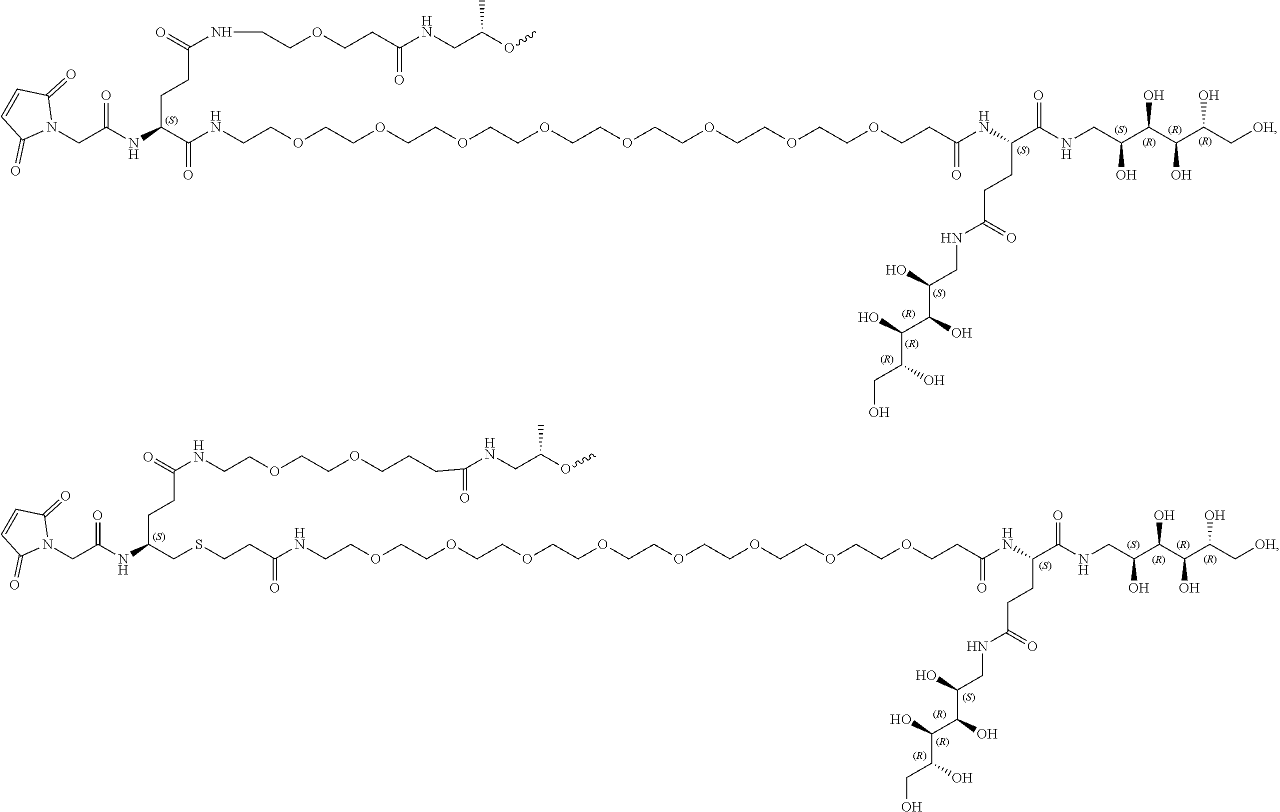

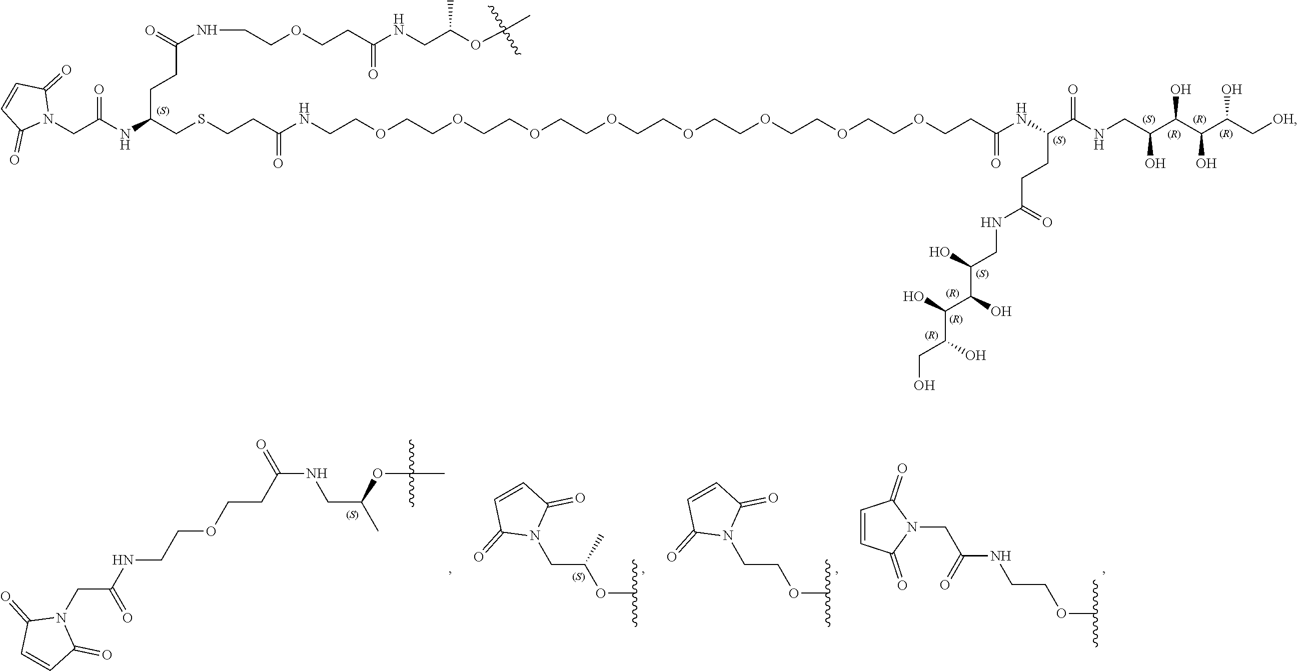

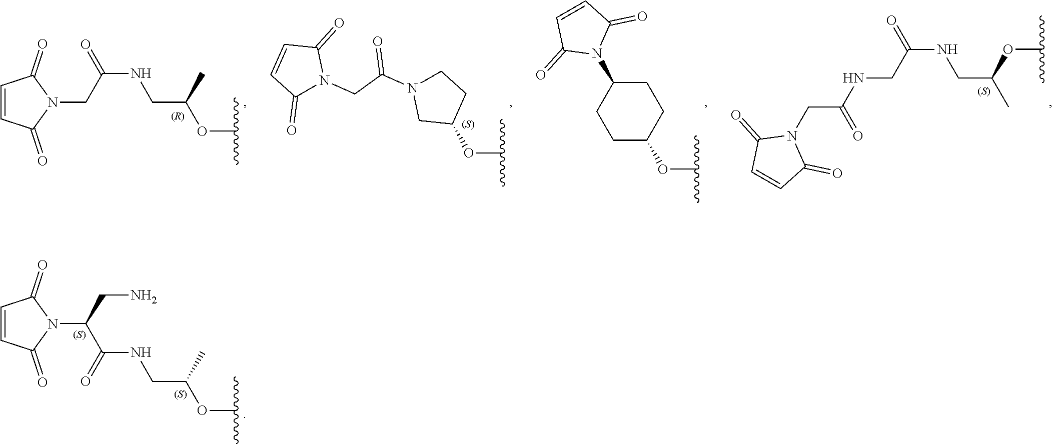

50. The compound-linker construct or conjugate of any one of the preceding claims, wherein the linker L¹ is selected from the group consisting of:

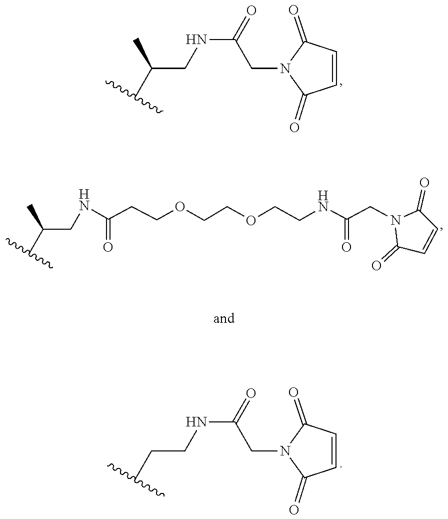 [SMILES: *[C@@H](C)CNC(=O)CN1C(=O)C=CC1=O ; *[C@@H](C)CNC(=O)CCOCCOCCNC(=O)CN1C(=O)C=CC1=O ; *CCNC(=O)CN1C(=O)C=CC1=O]

51. The compound-linker construct or conjugate of any one of the preceding claims, wherein the linker L¹ is selected from the group consisting of:

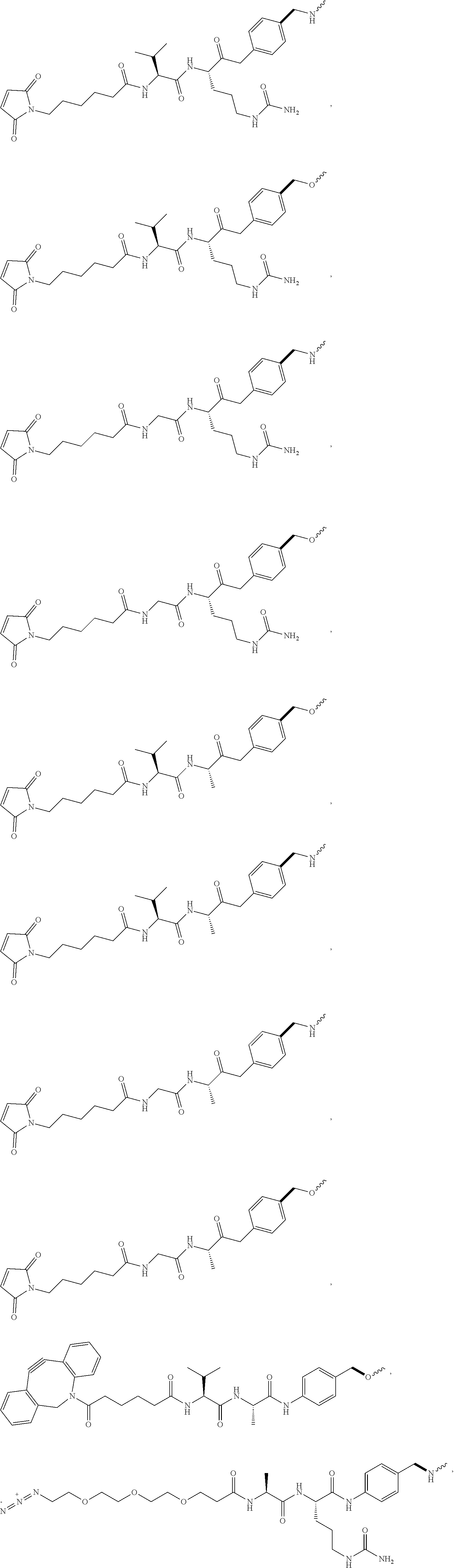

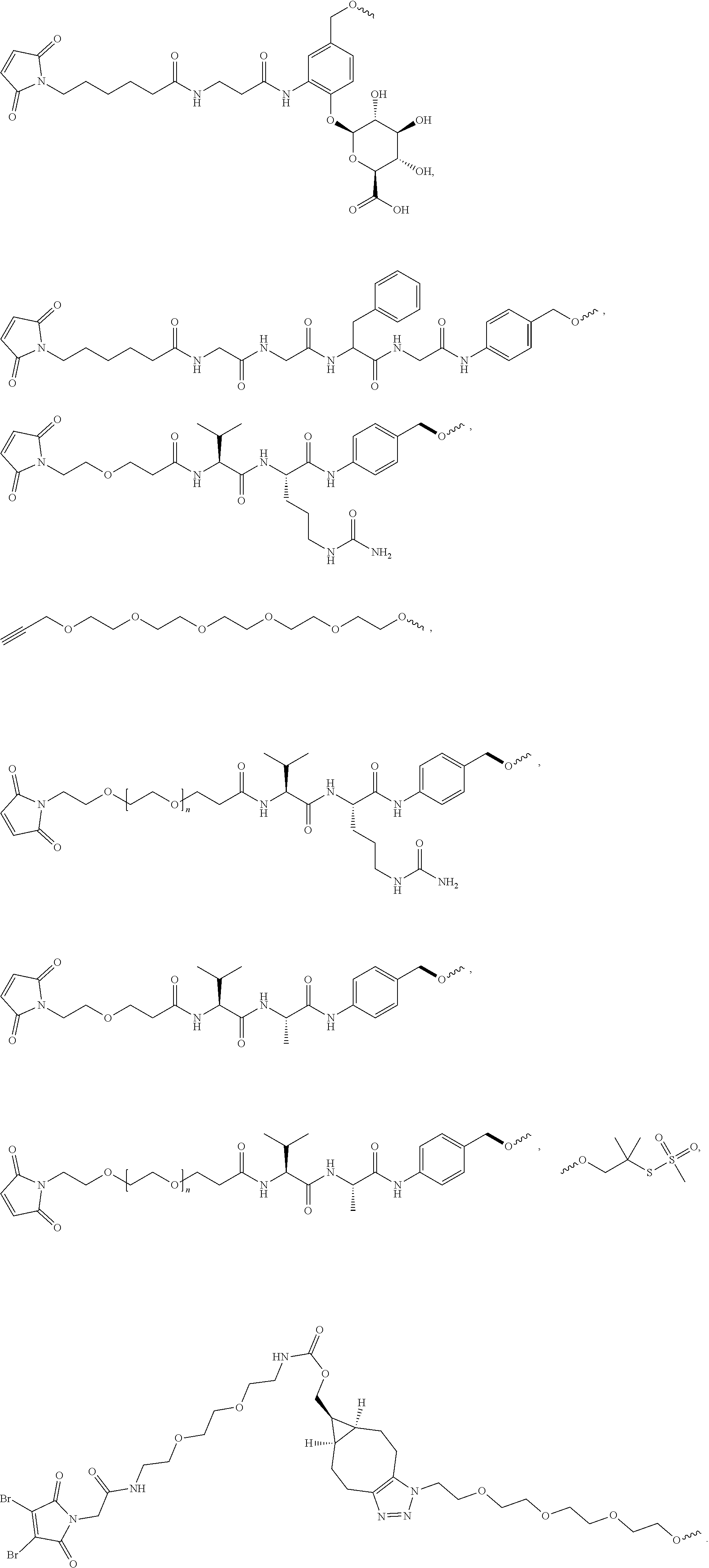

52. The compound-linker construct or conjugate of any one of the preceding claims, wherein the compound-linker construct is selected from:

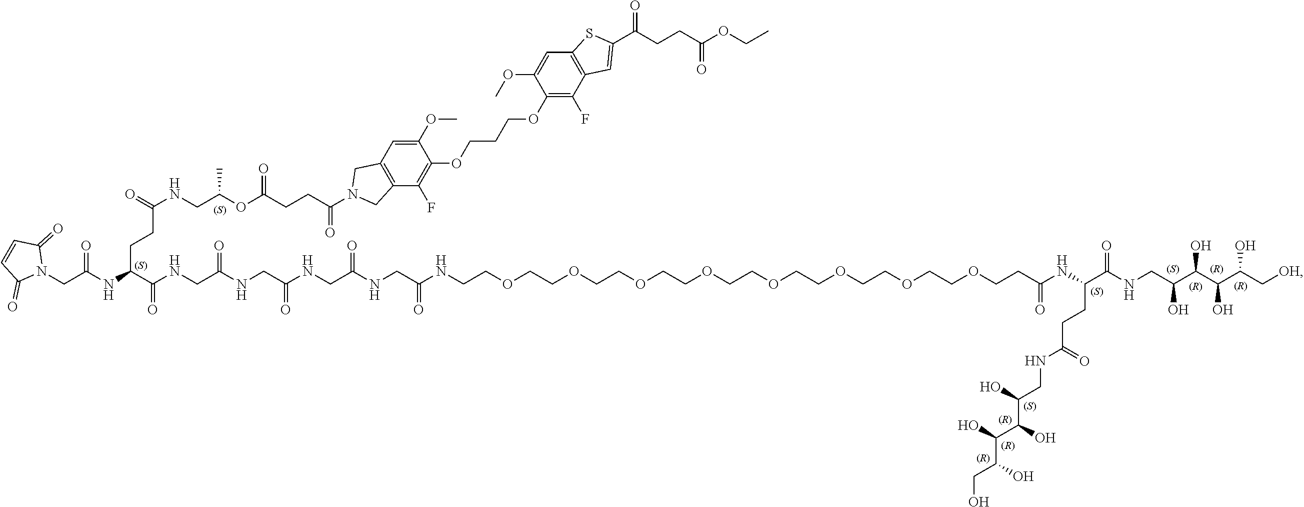

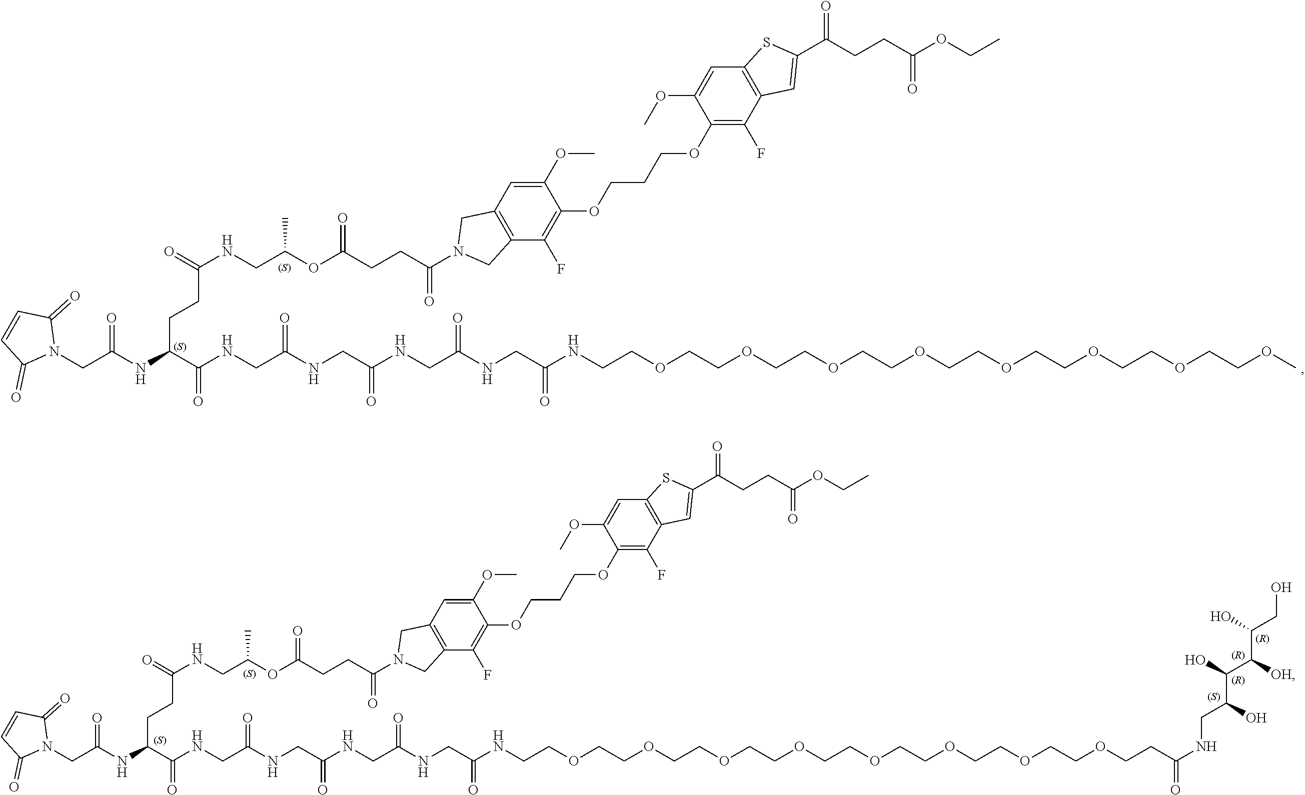

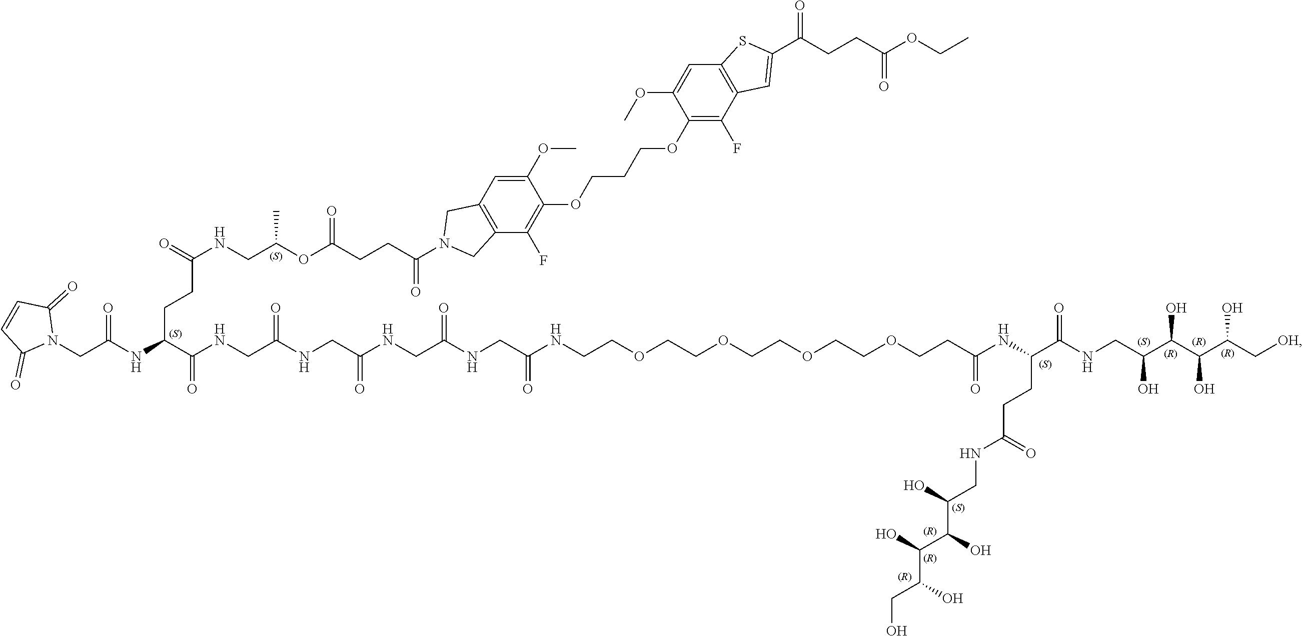

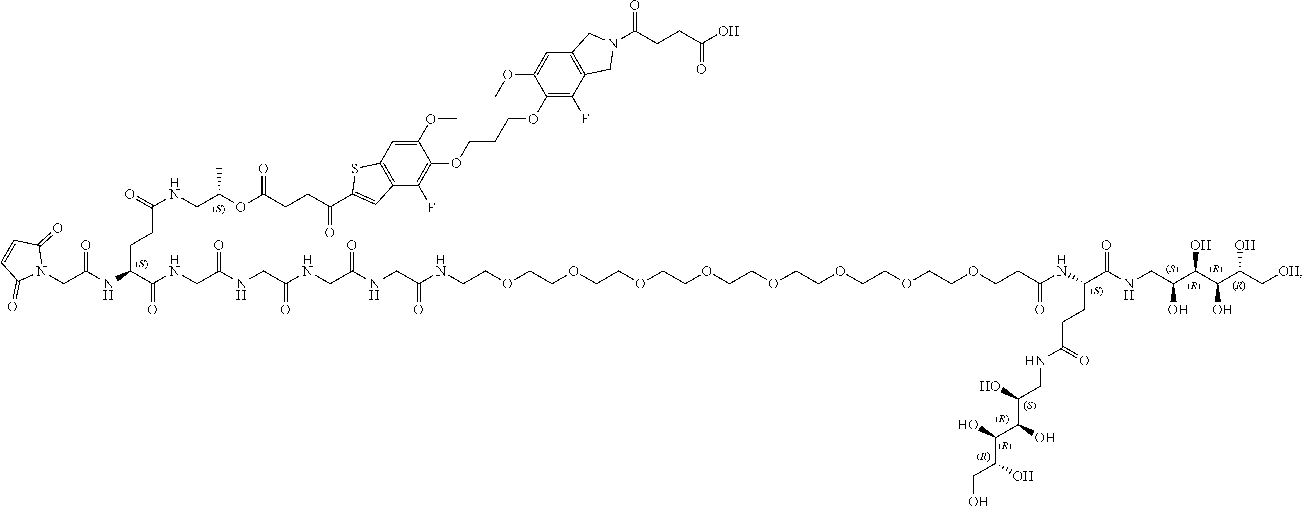

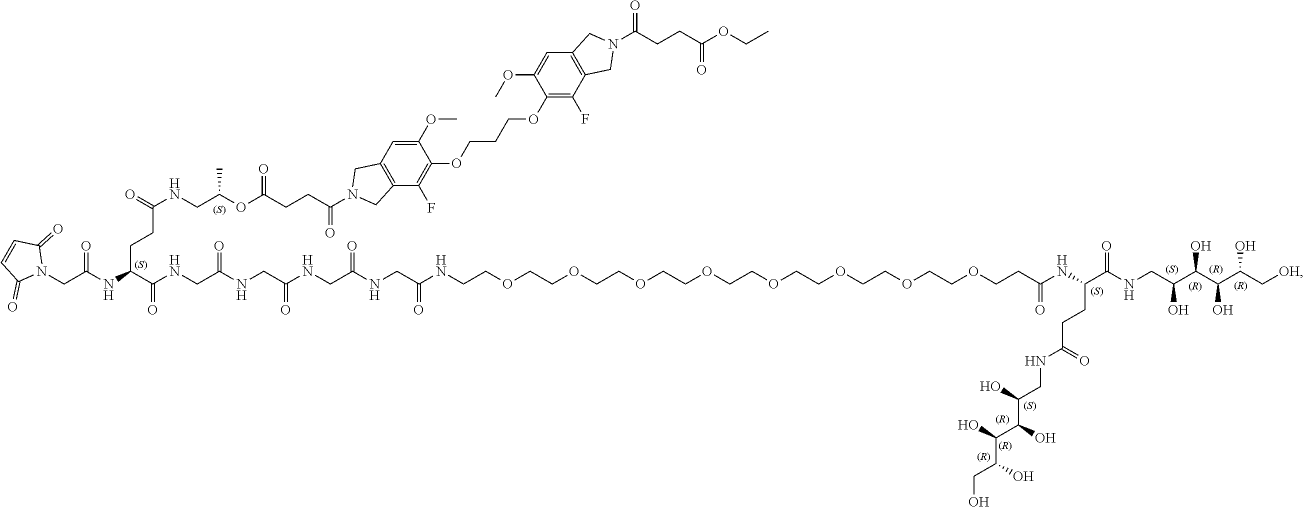

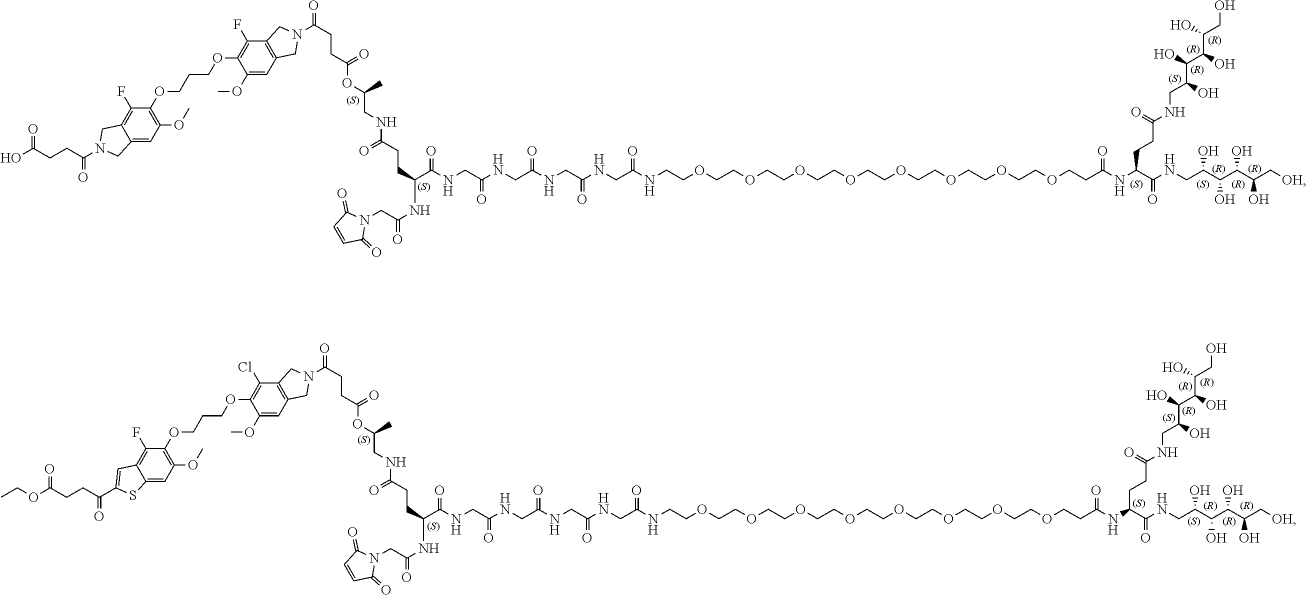

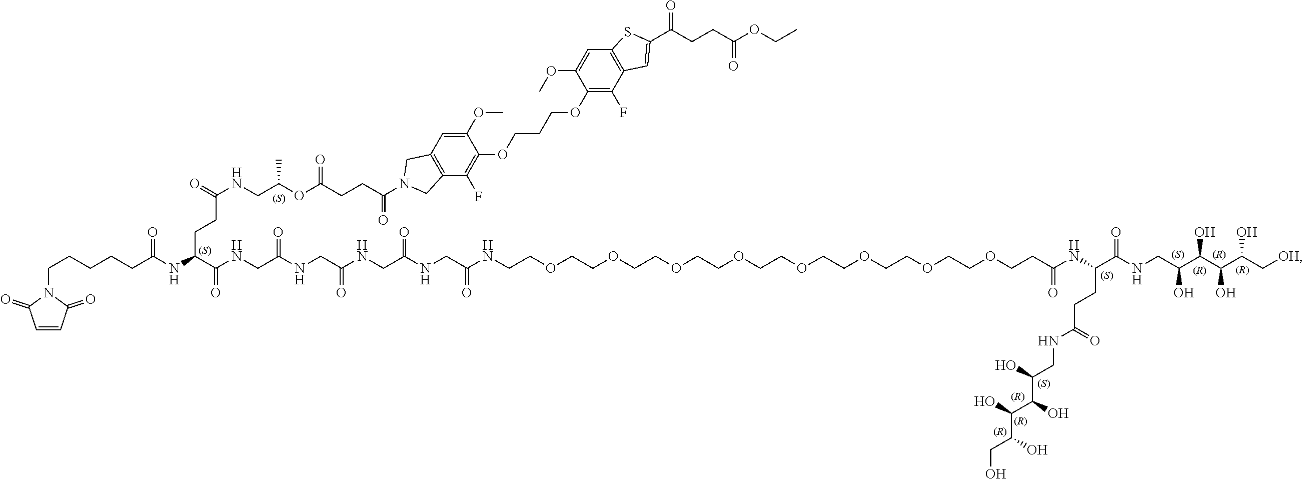

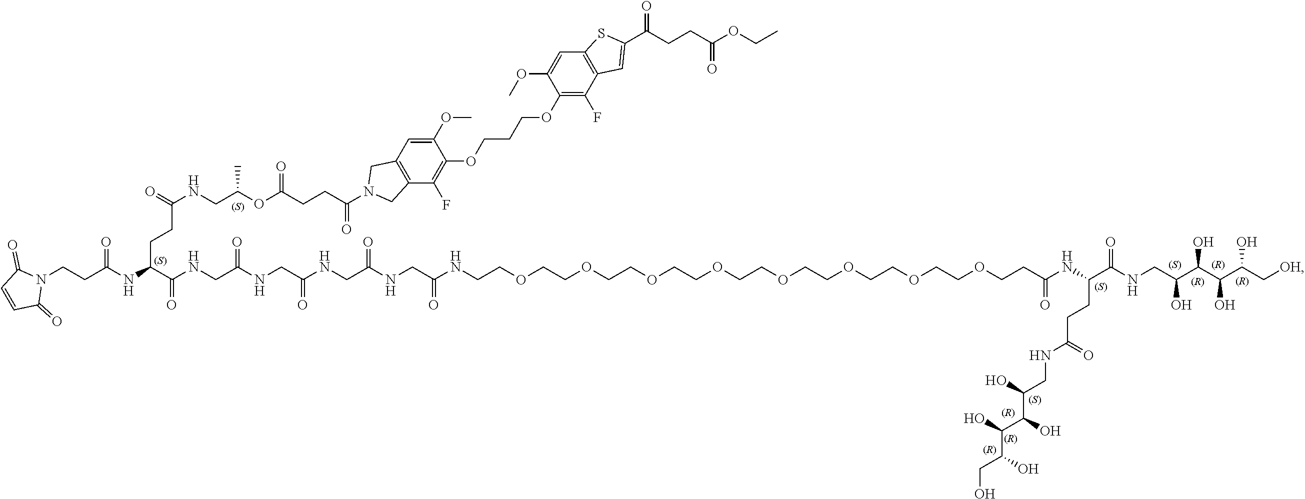

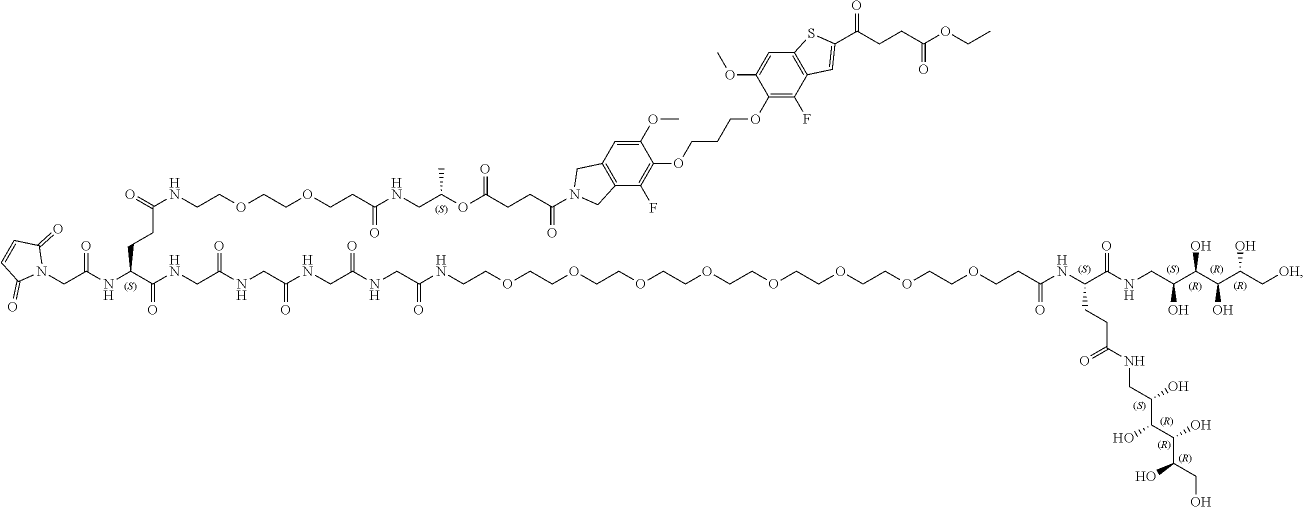

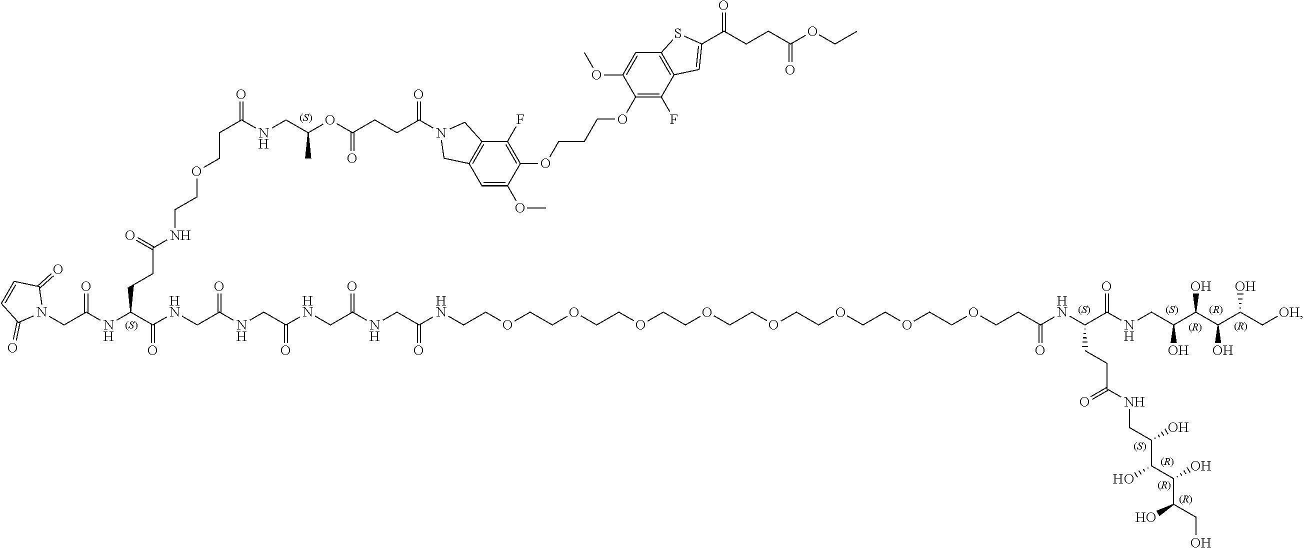

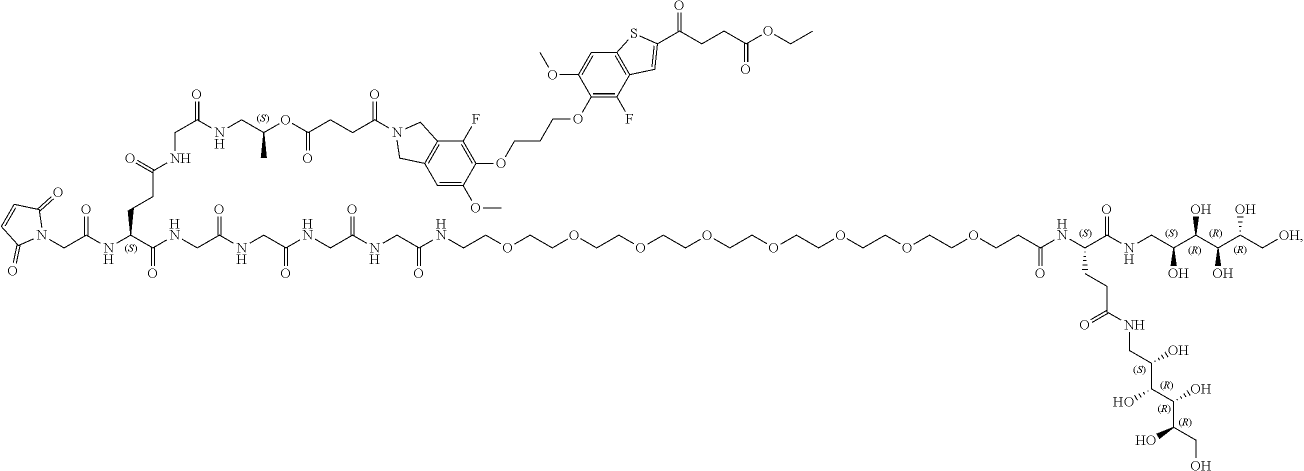

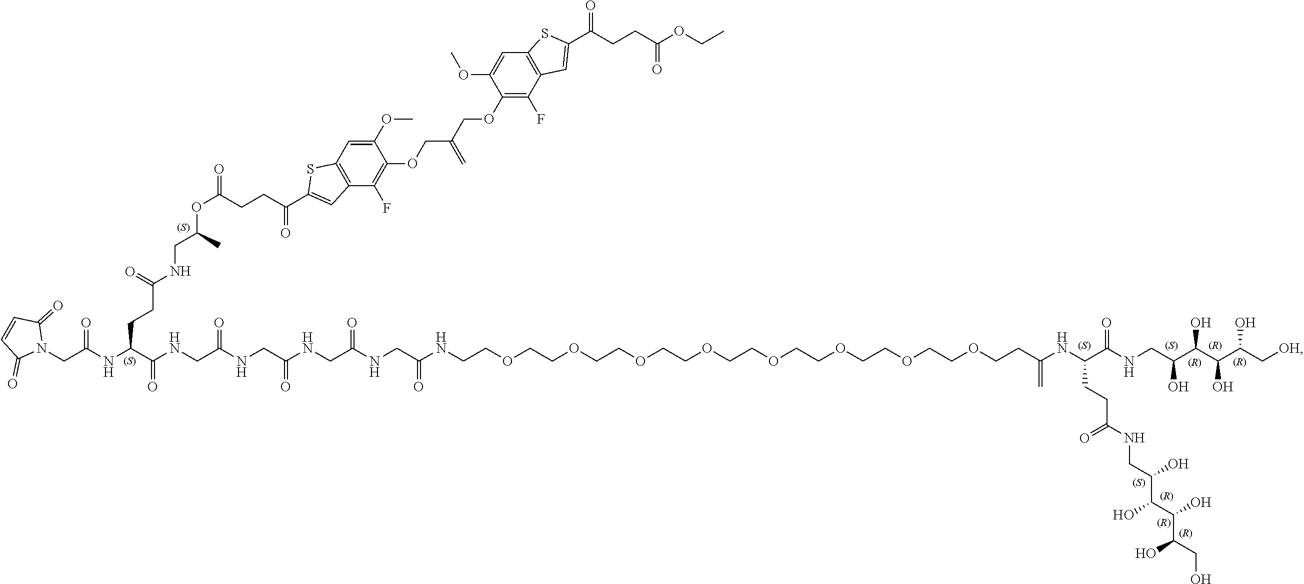

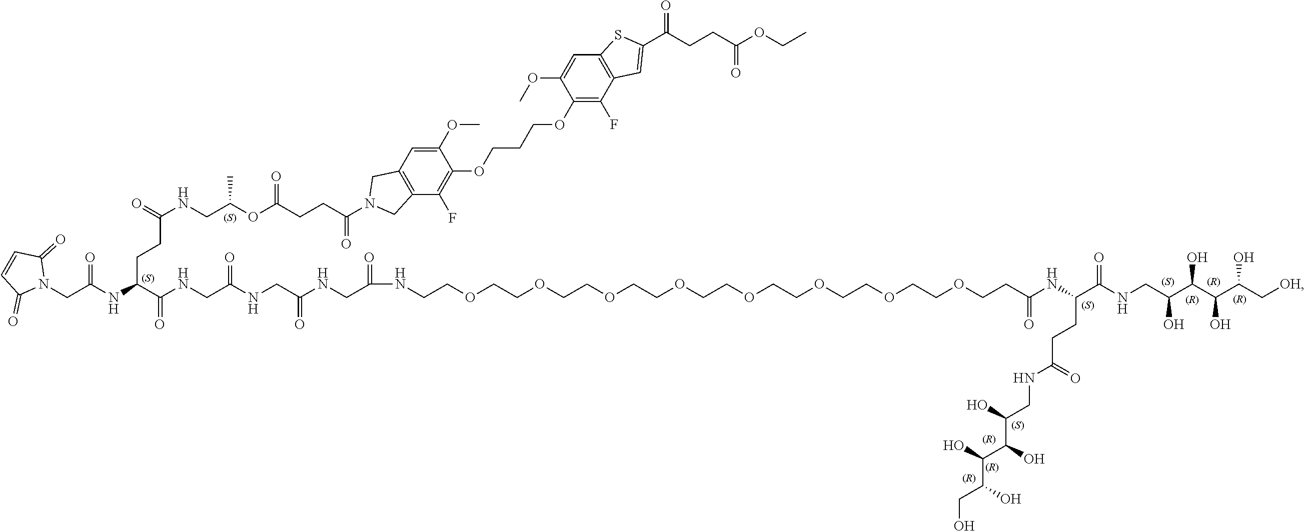

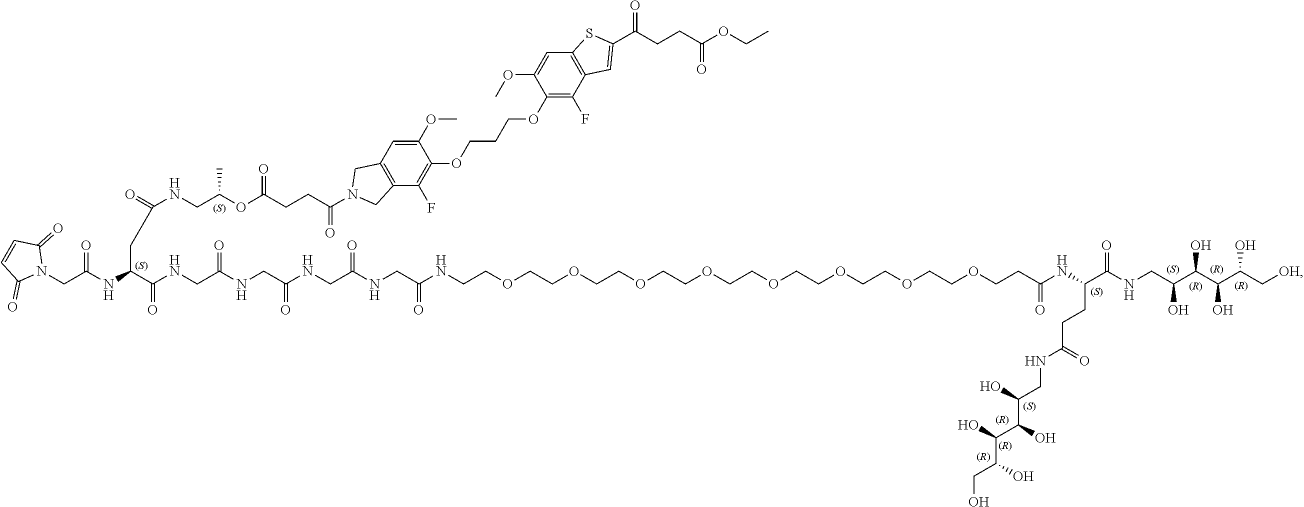

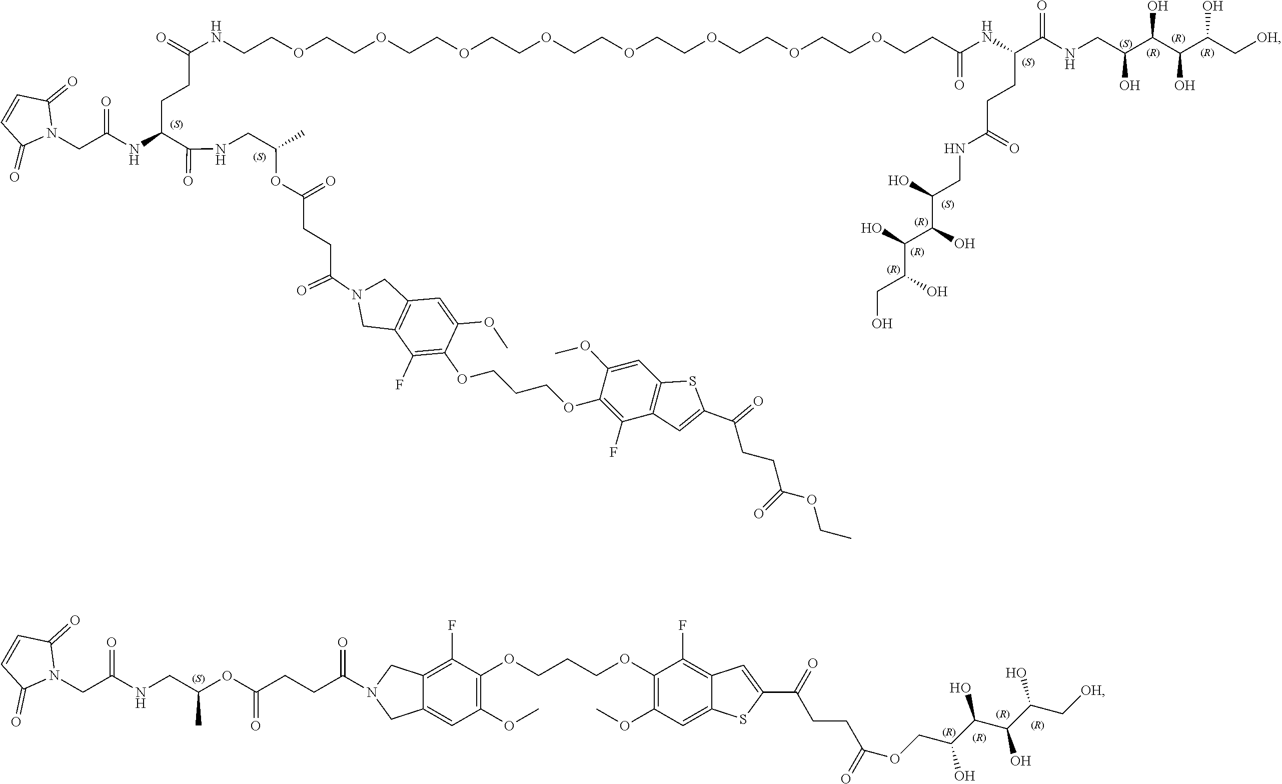

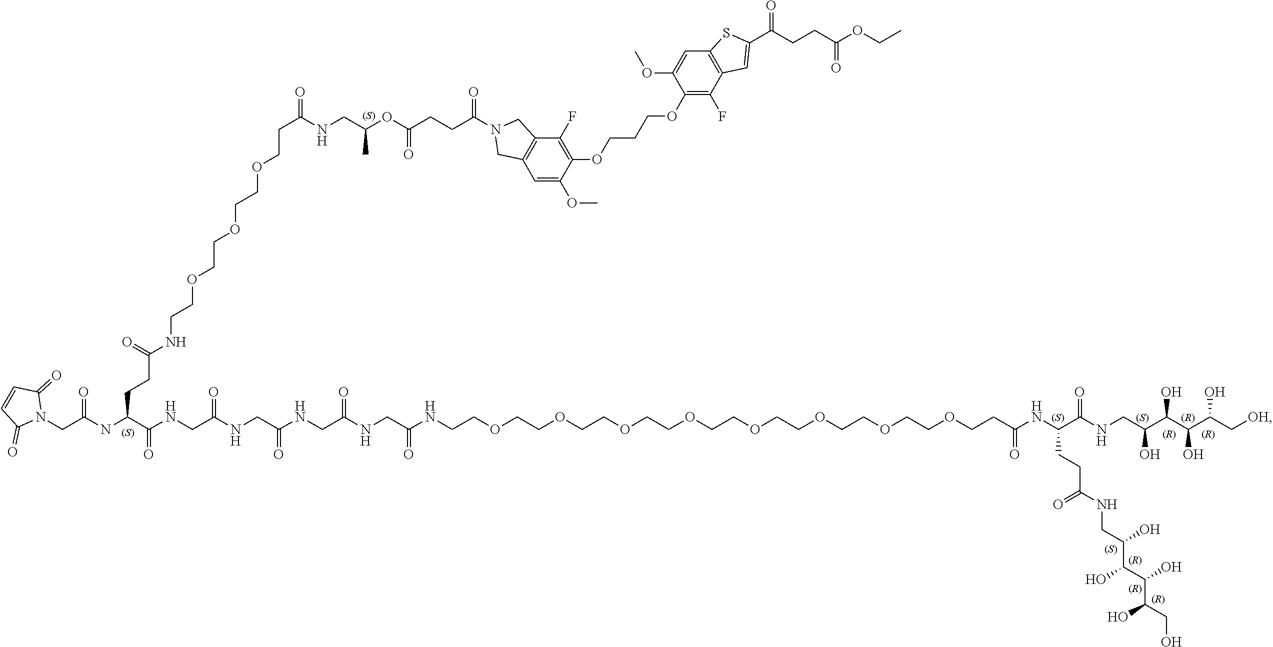

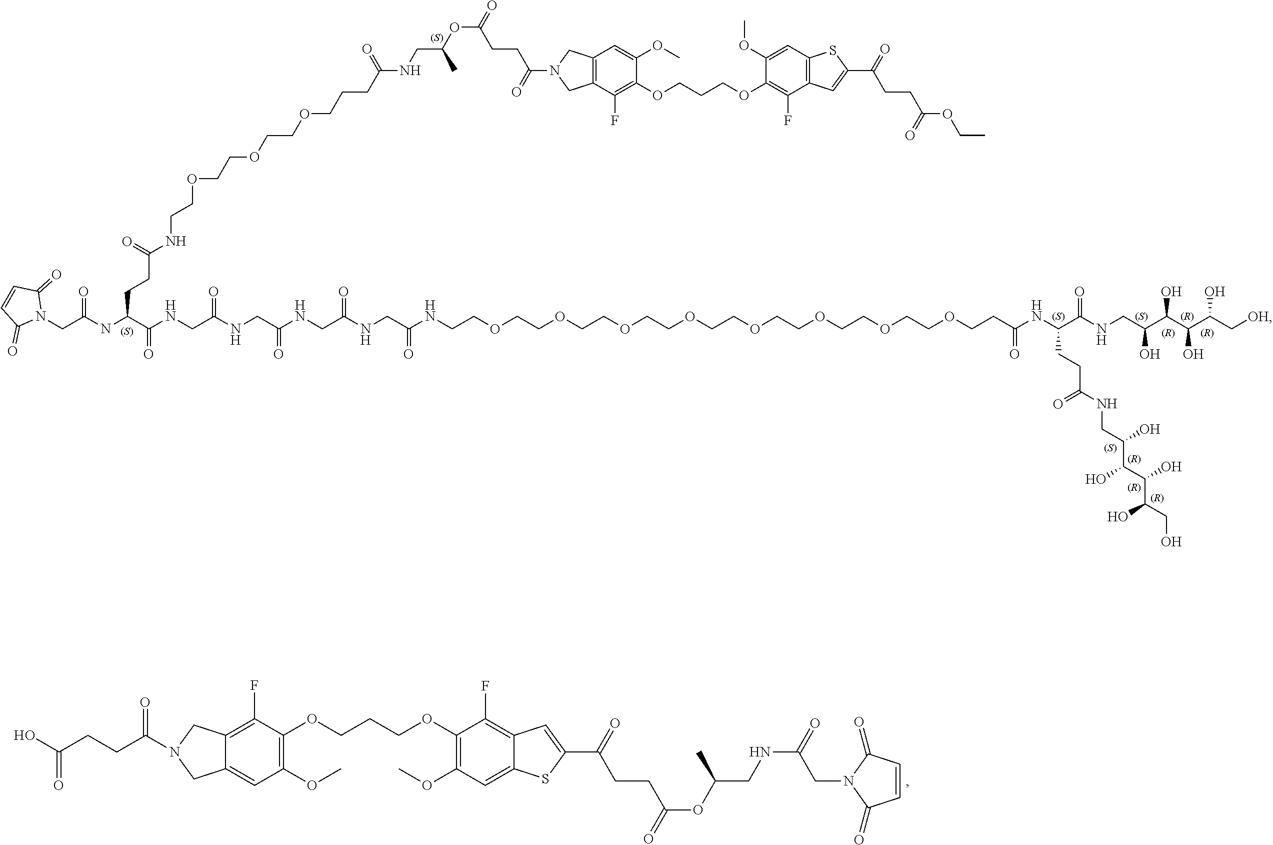

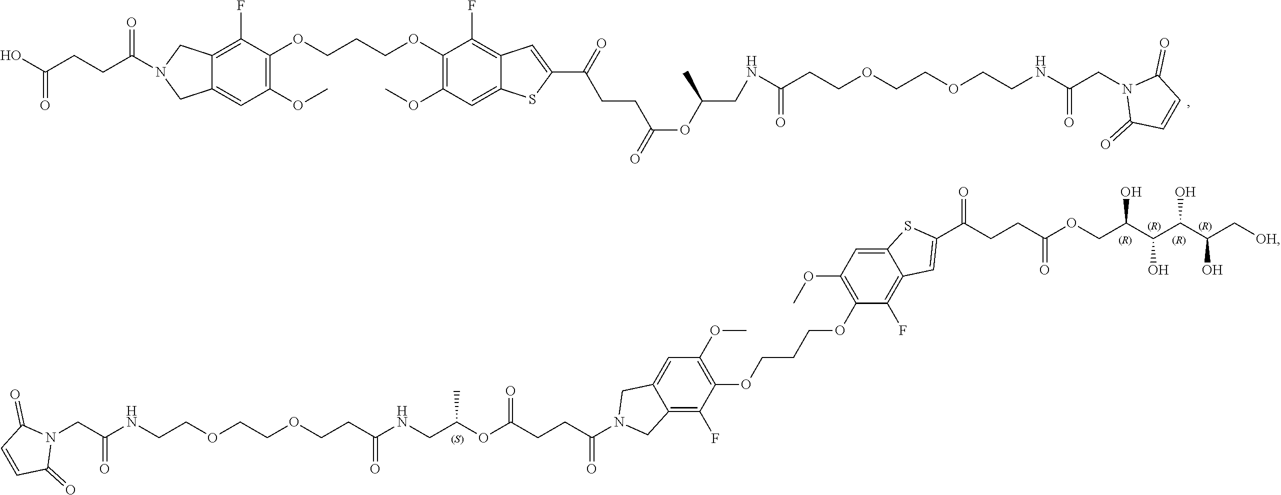

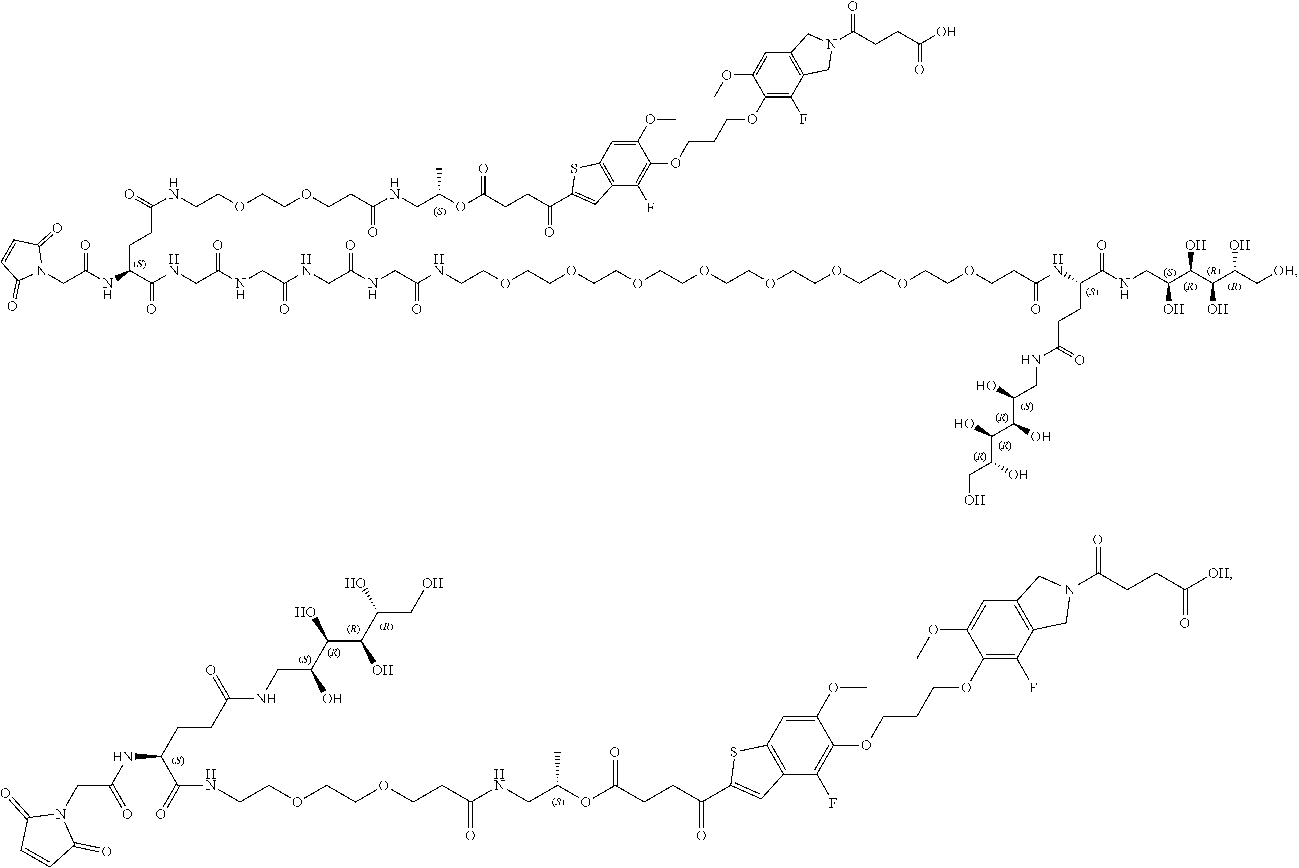

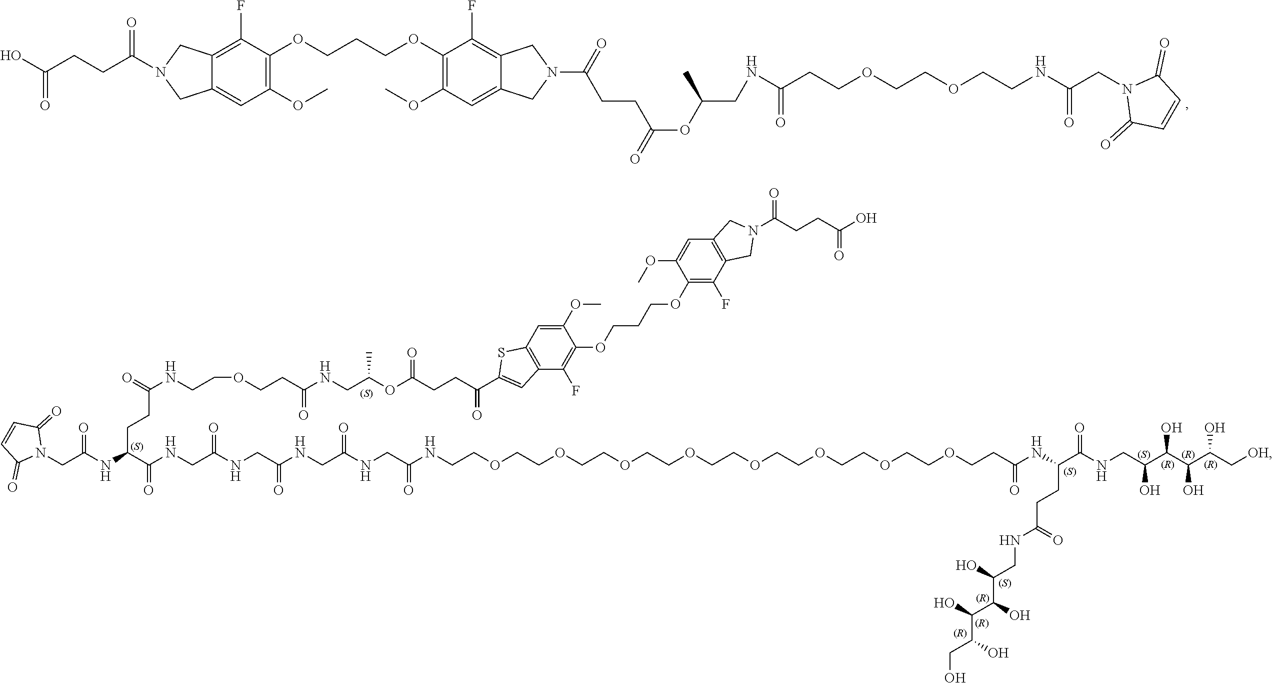

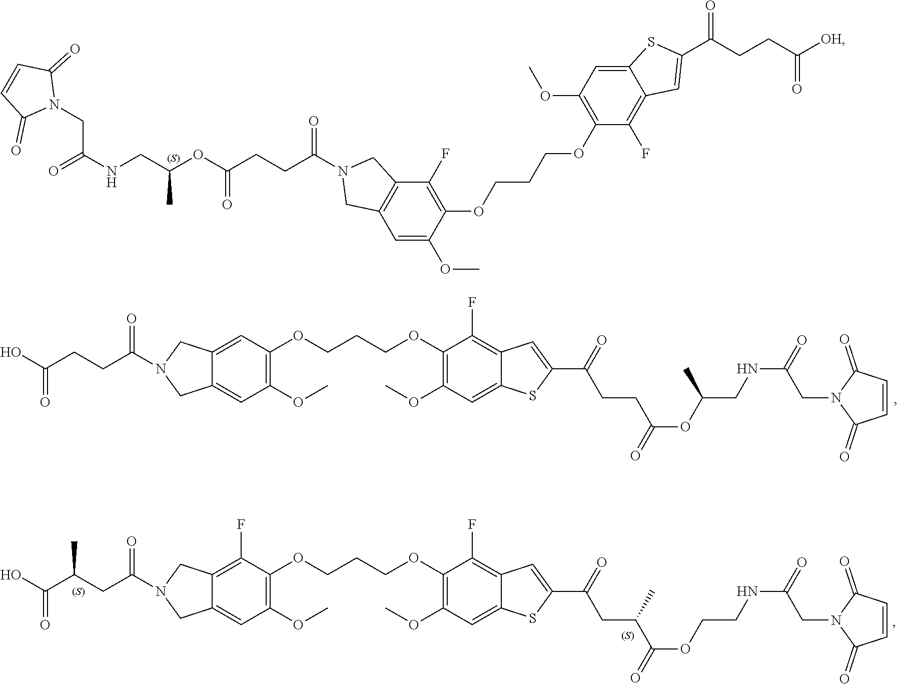

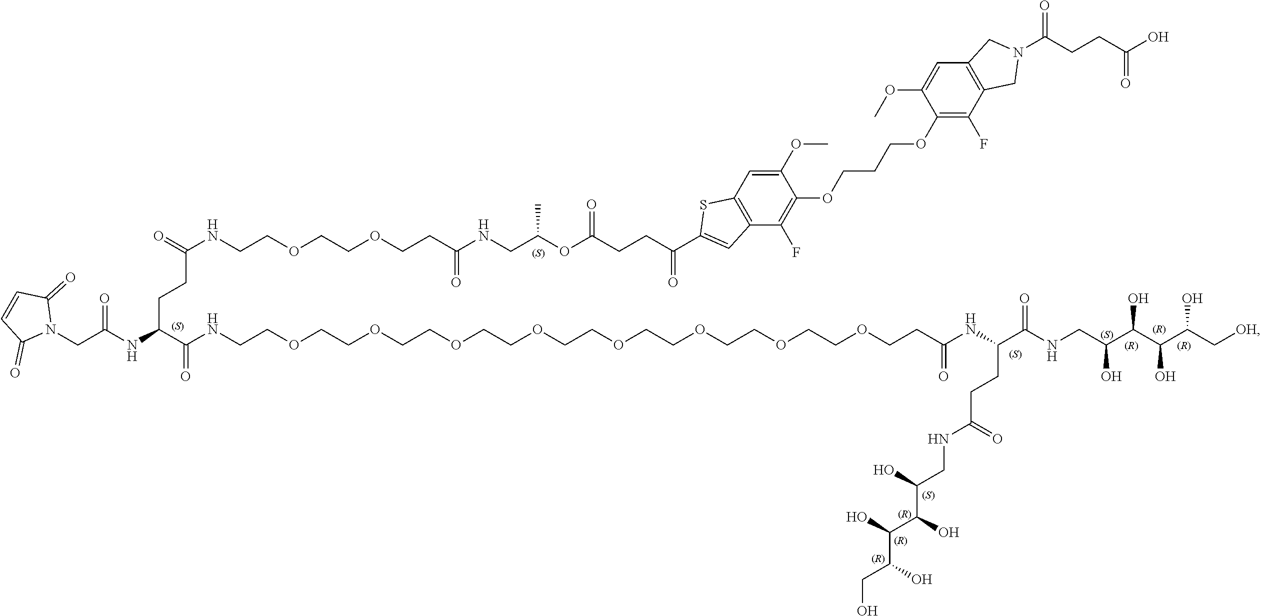

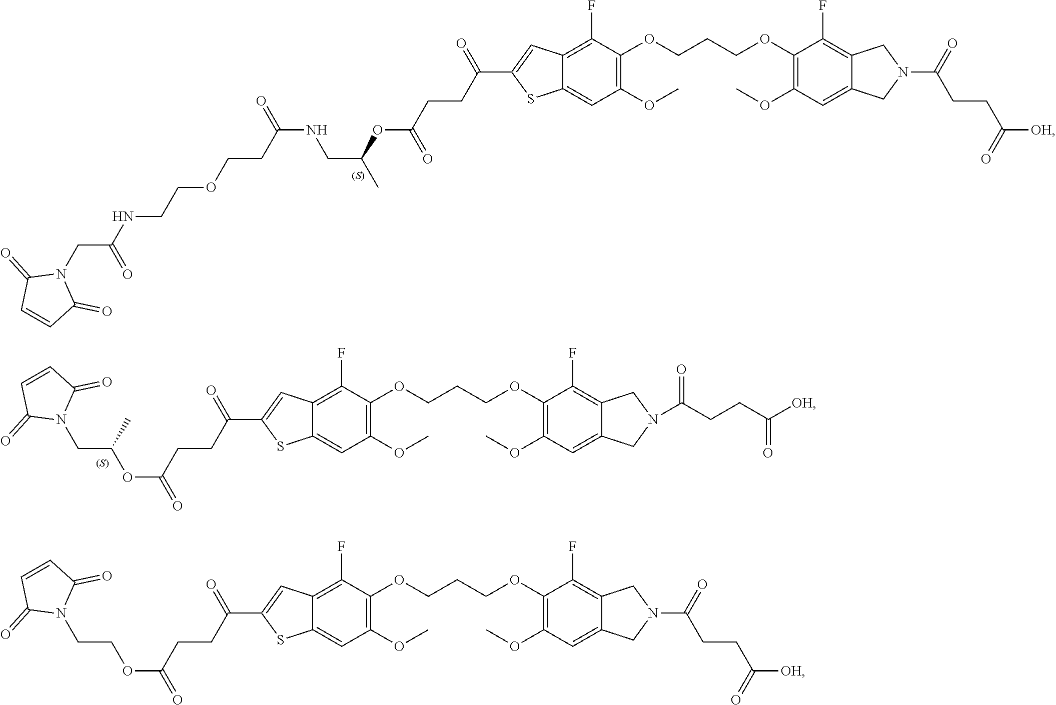

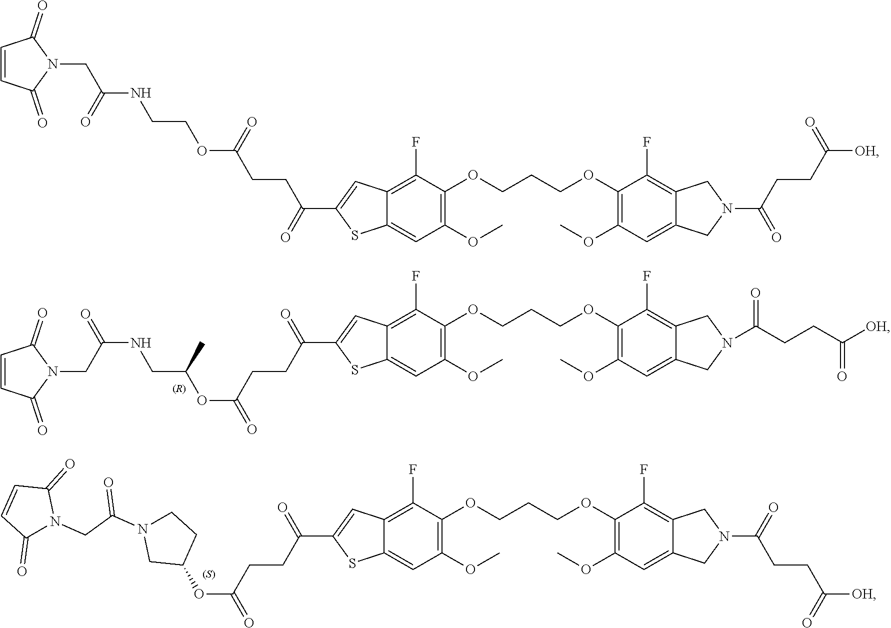

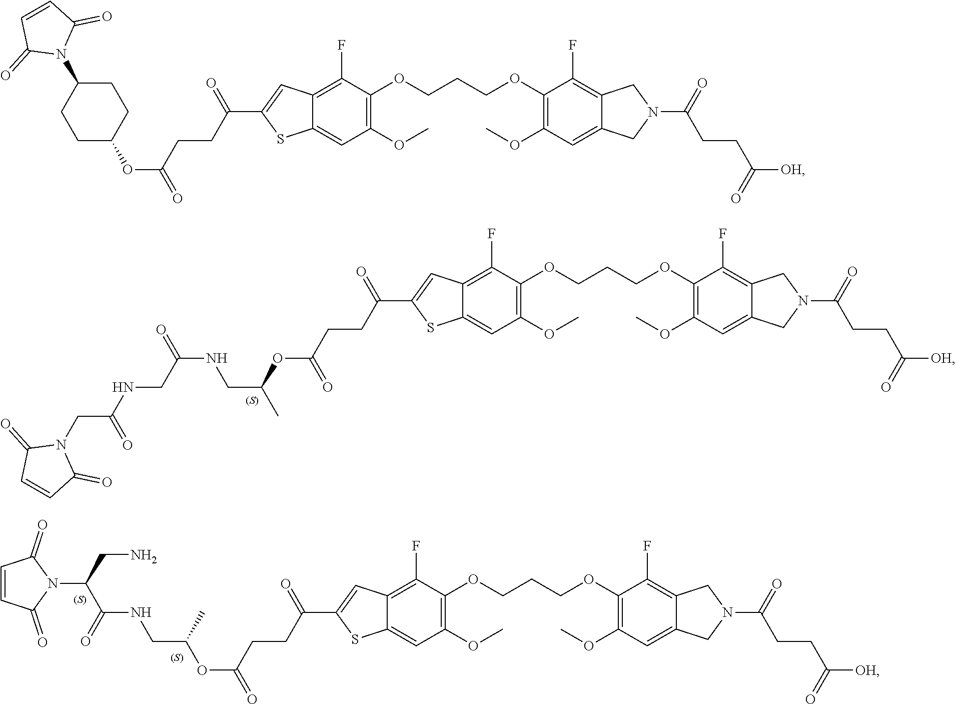

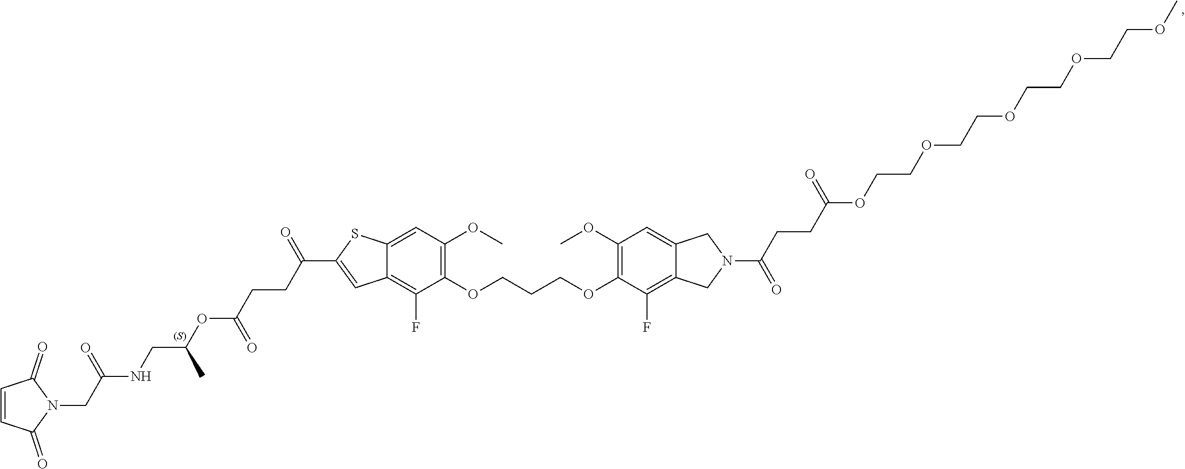

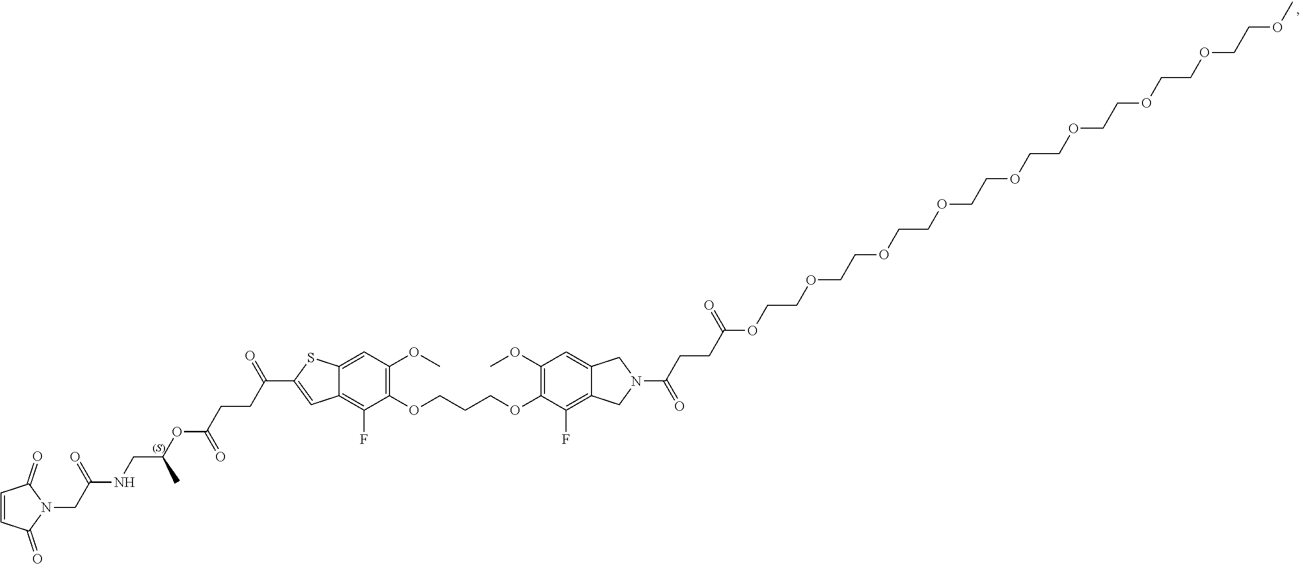

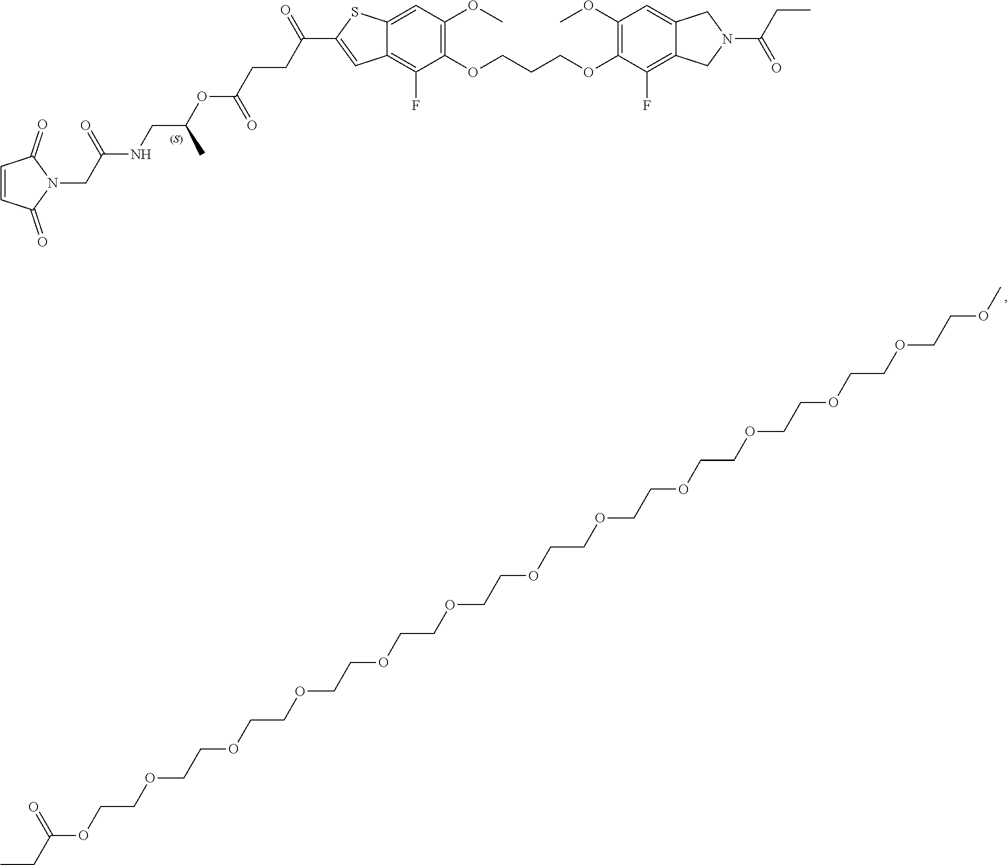

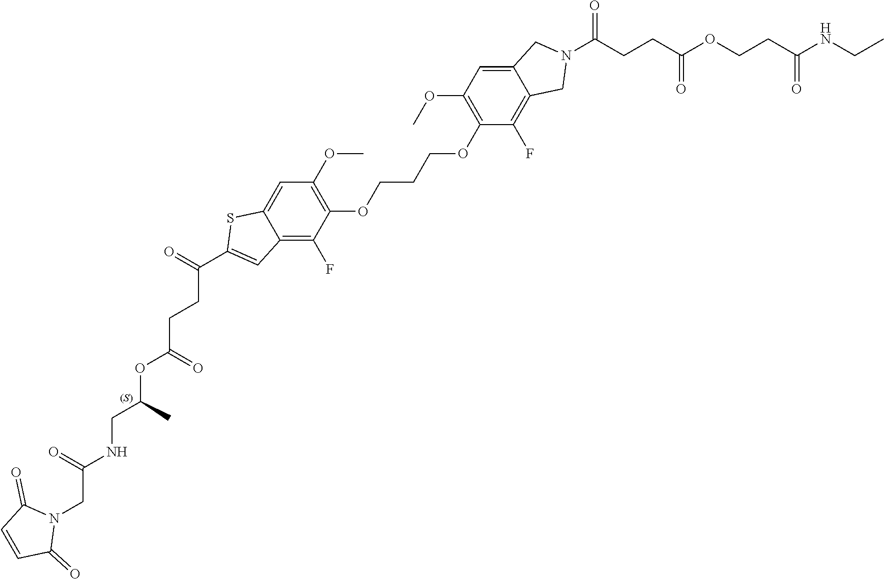

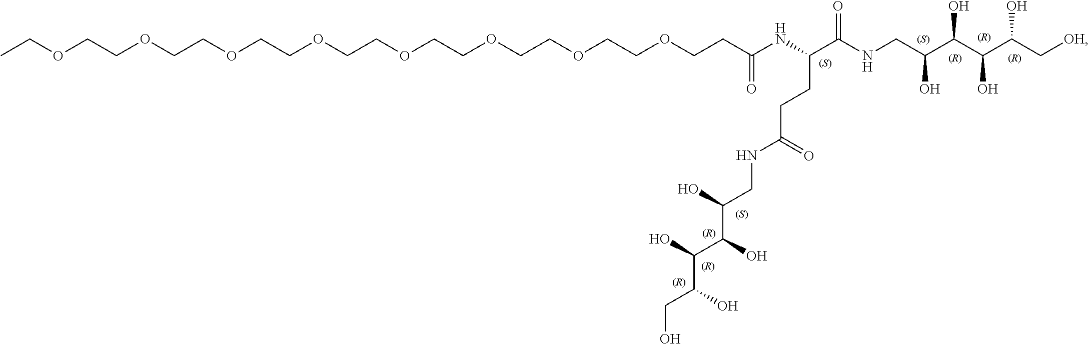

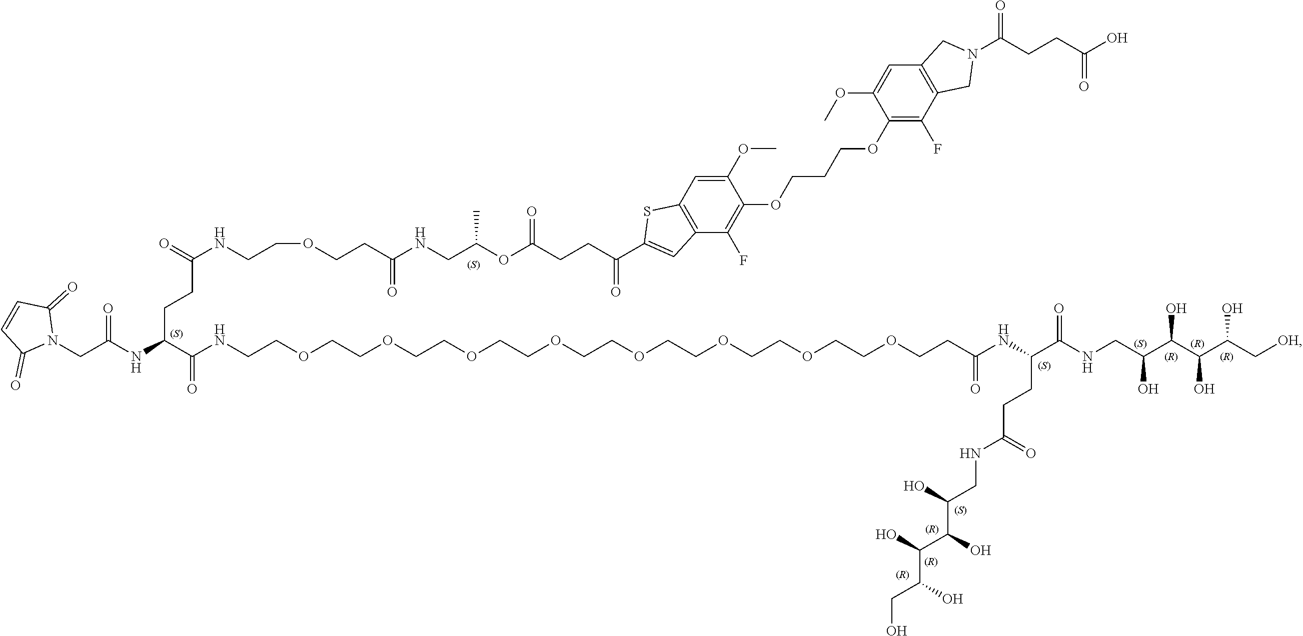

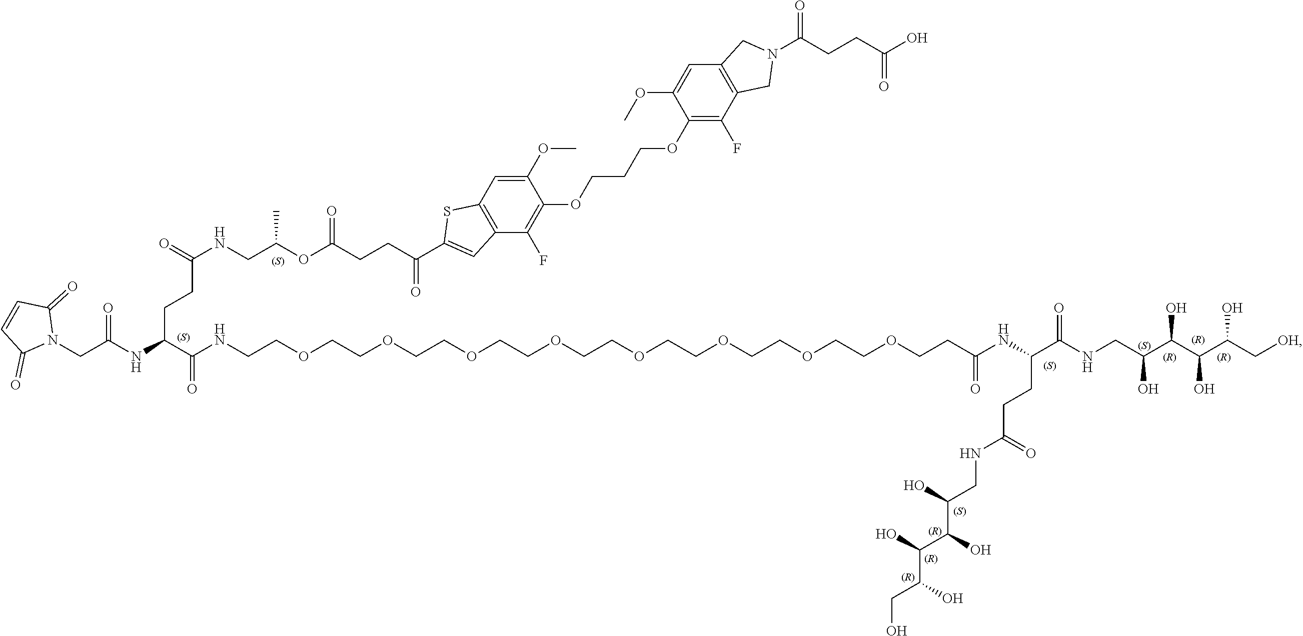

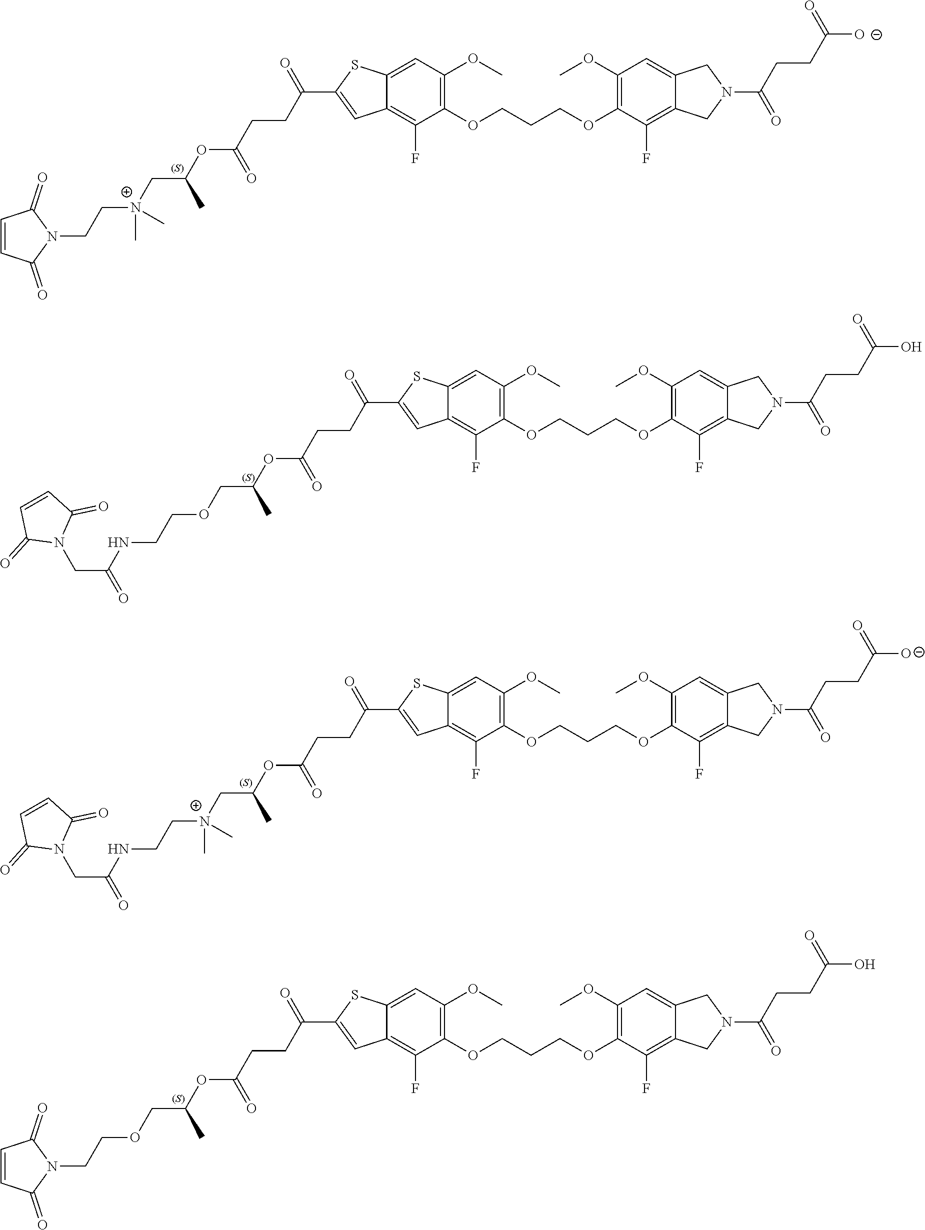

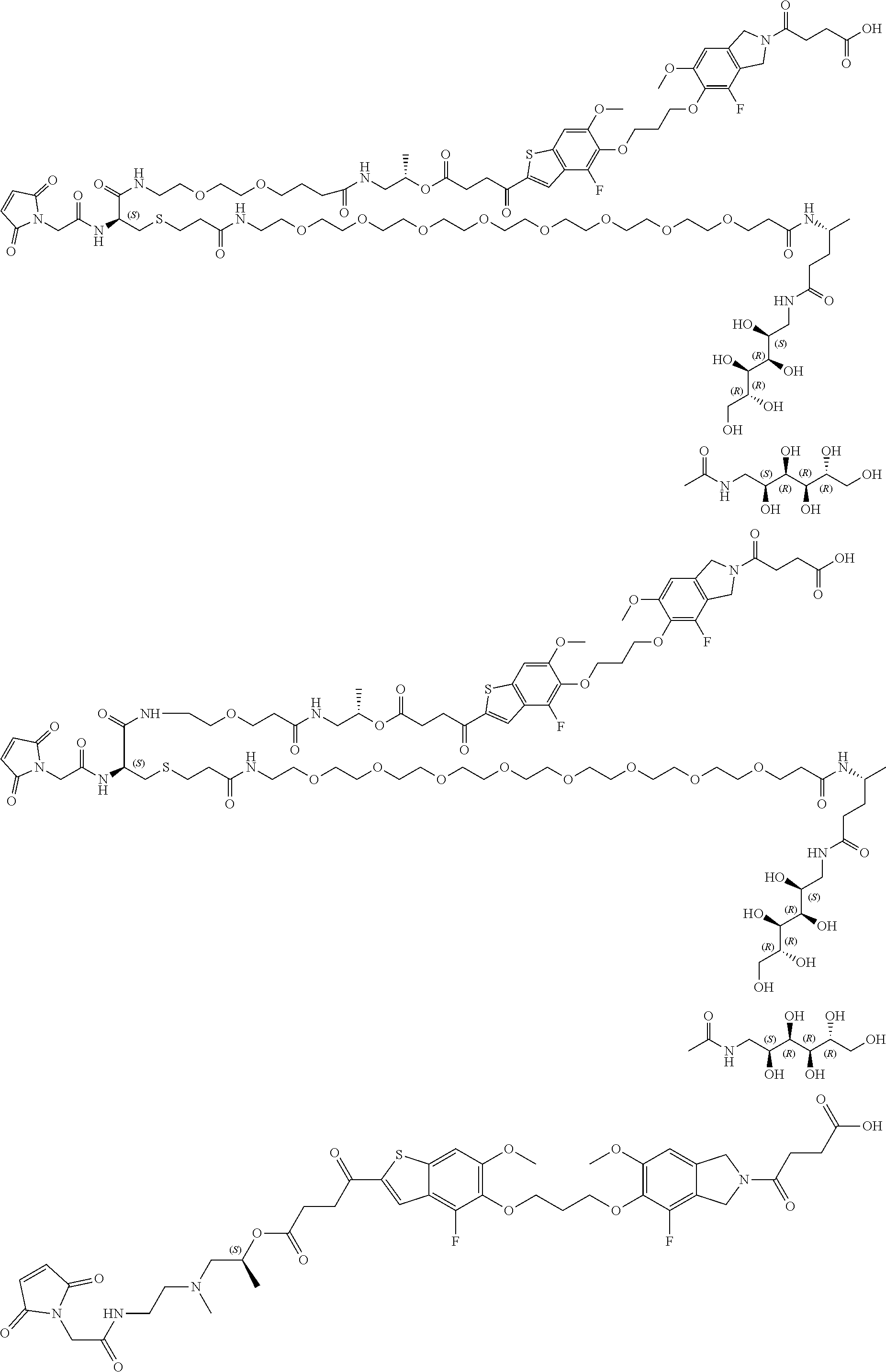

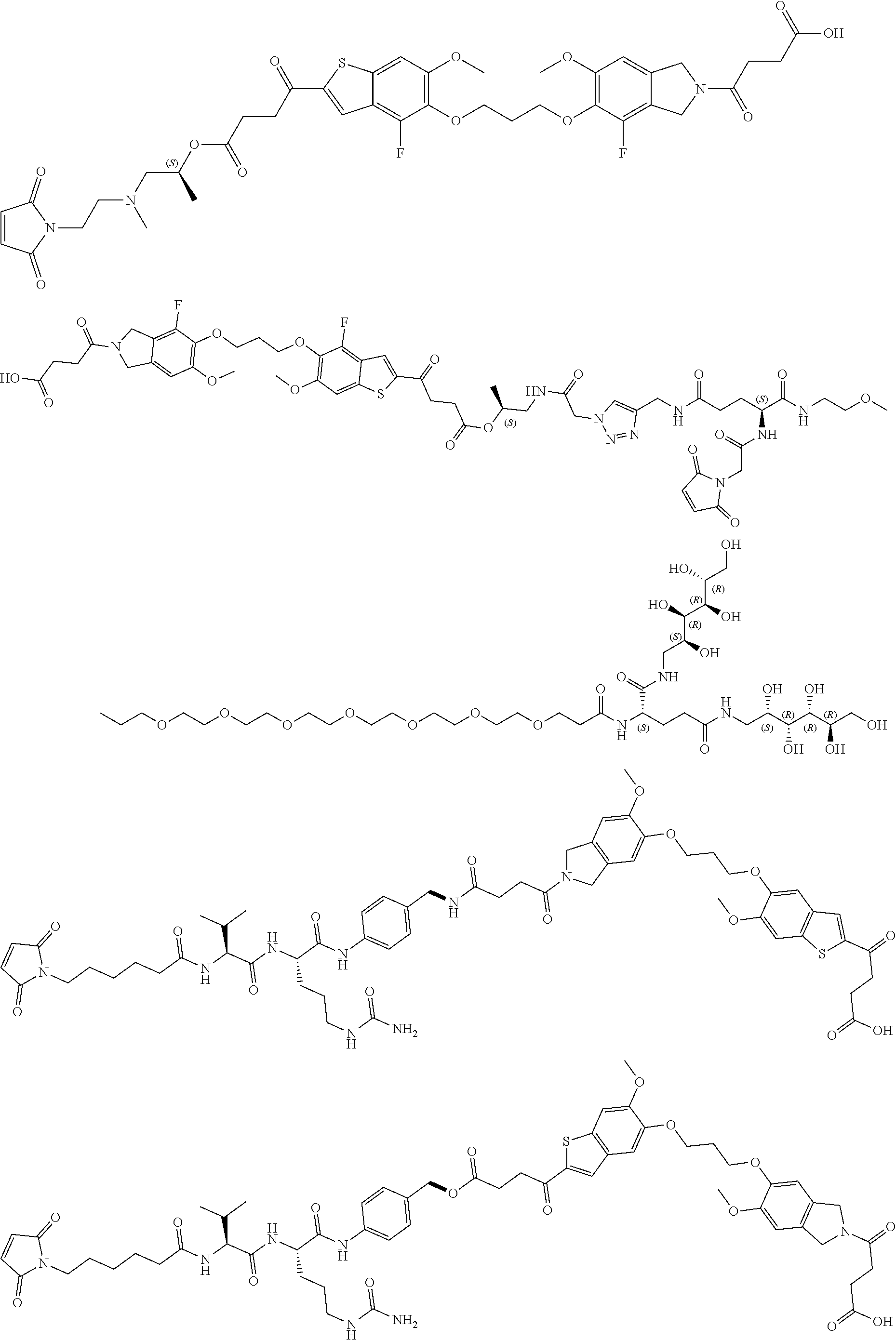

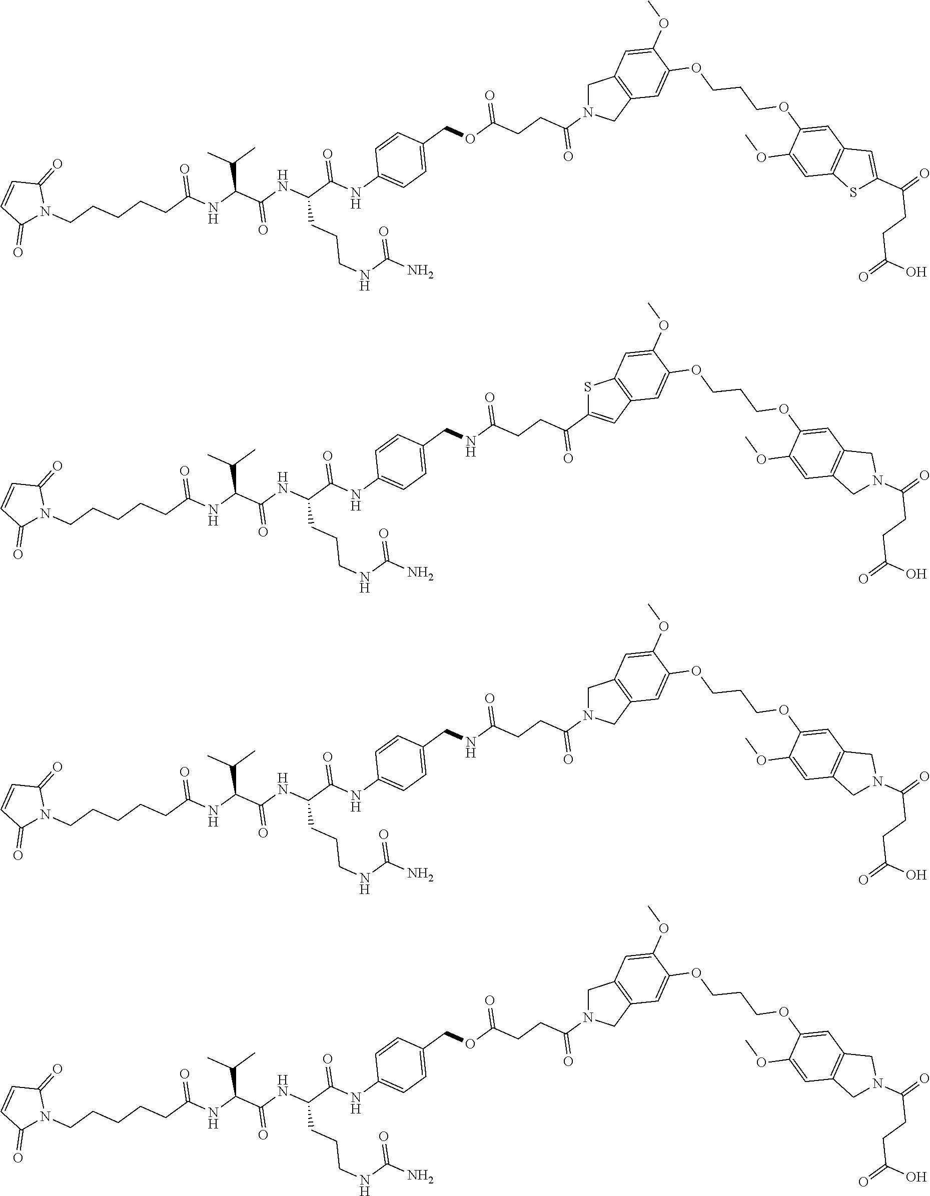

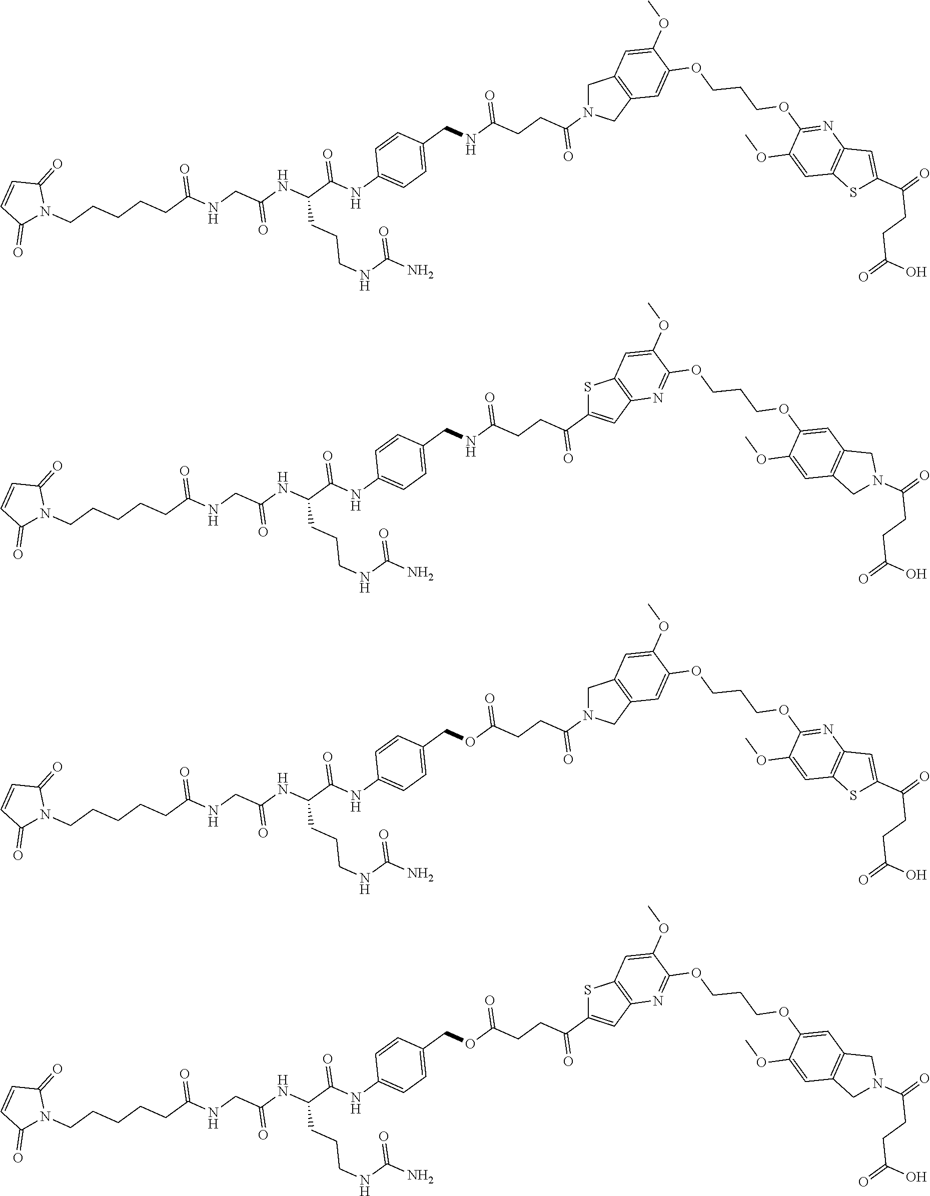

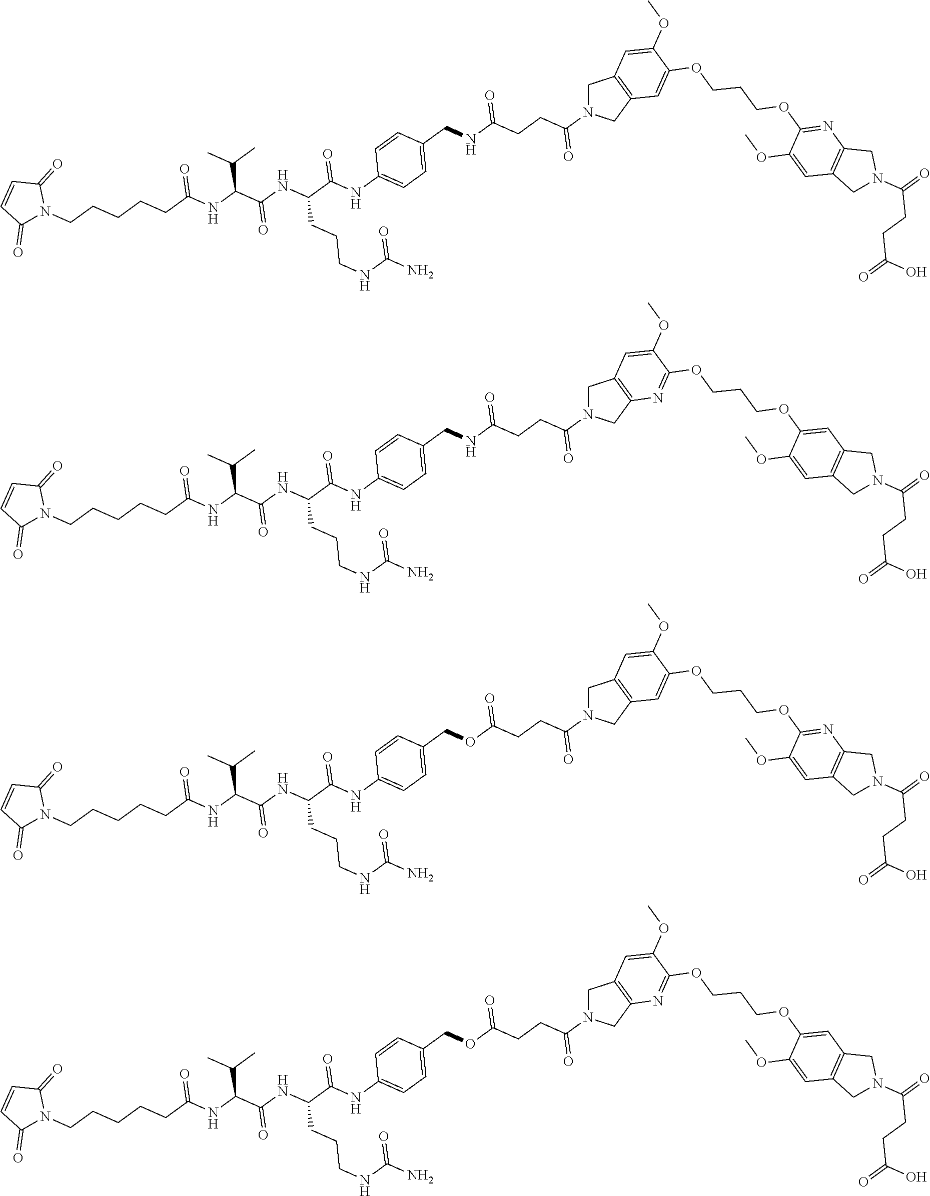

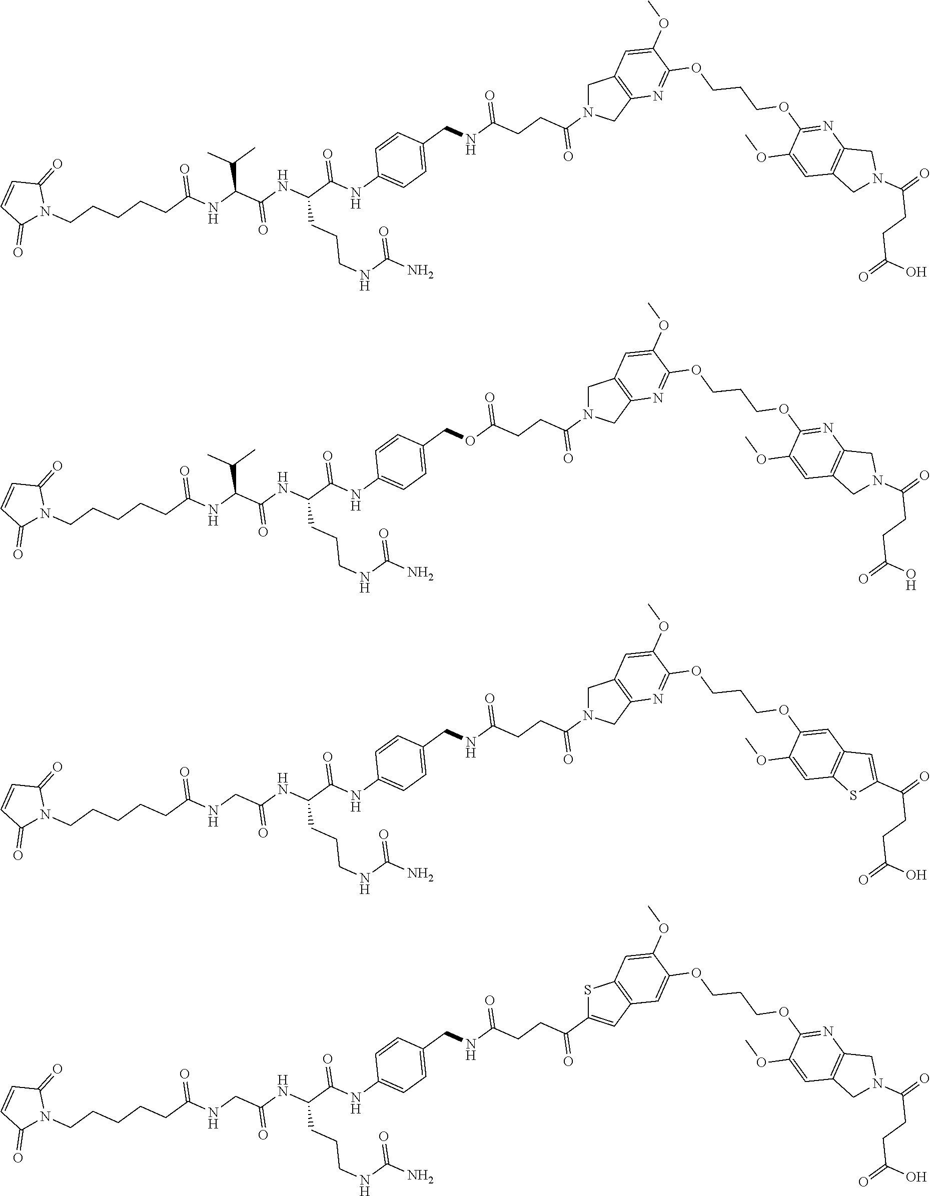

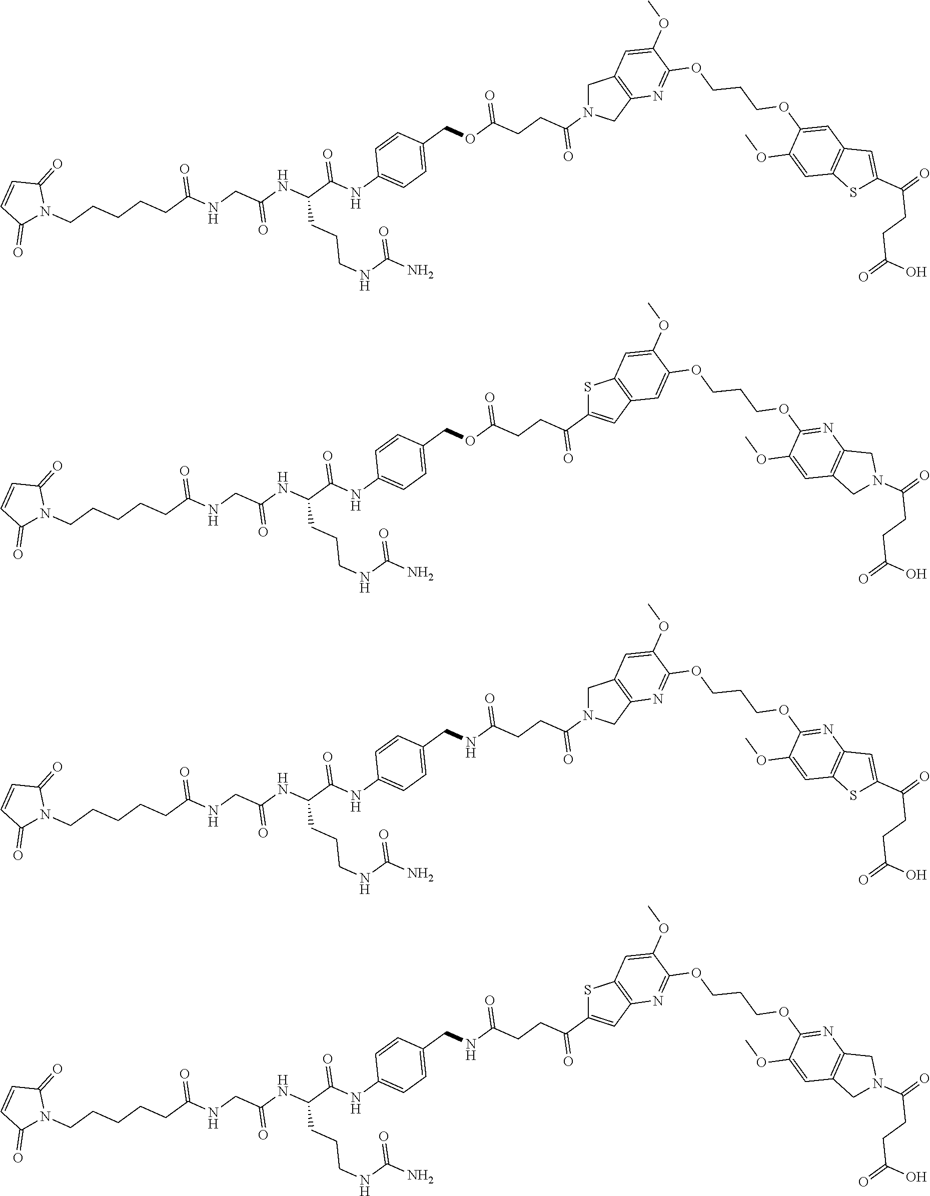

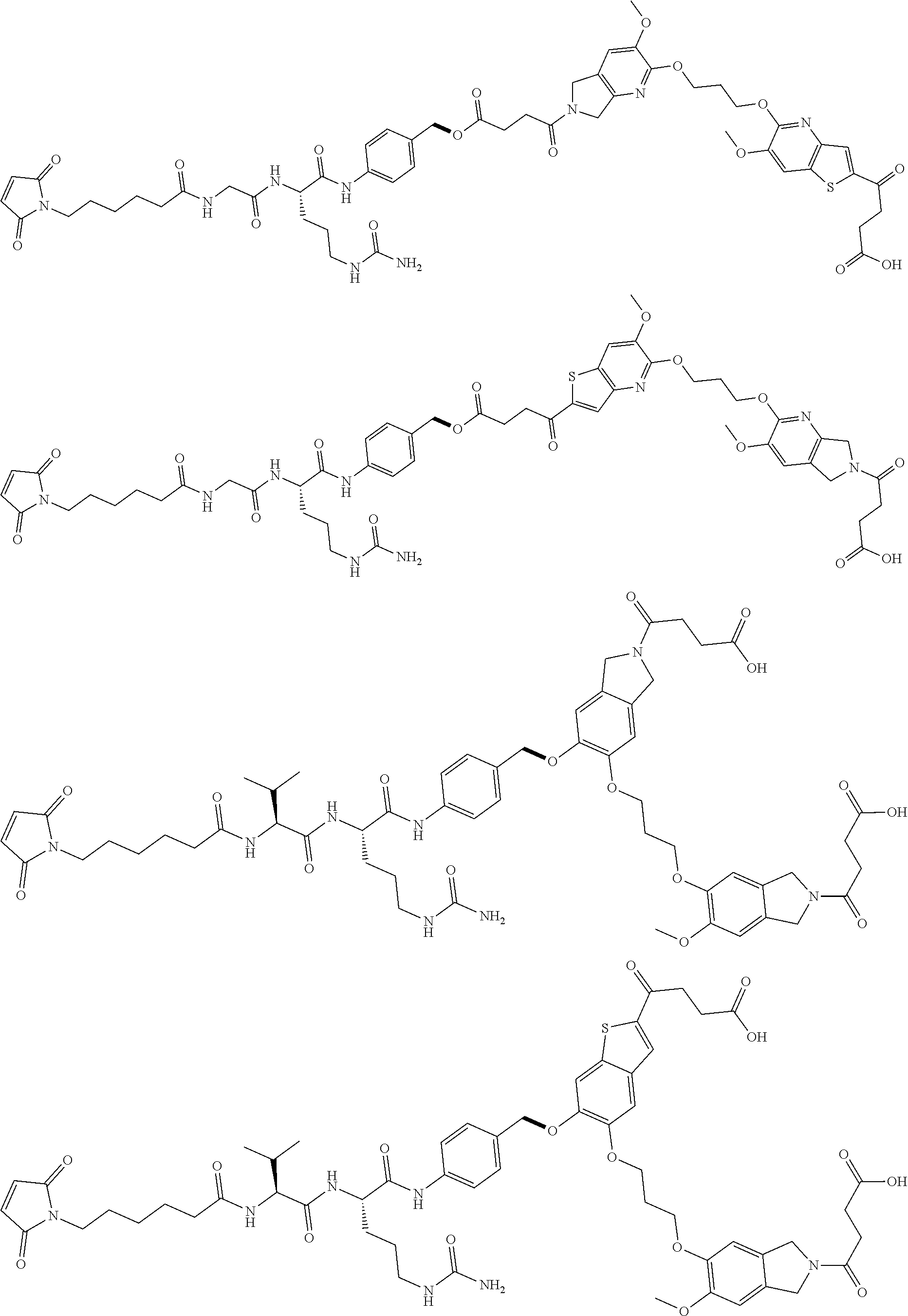

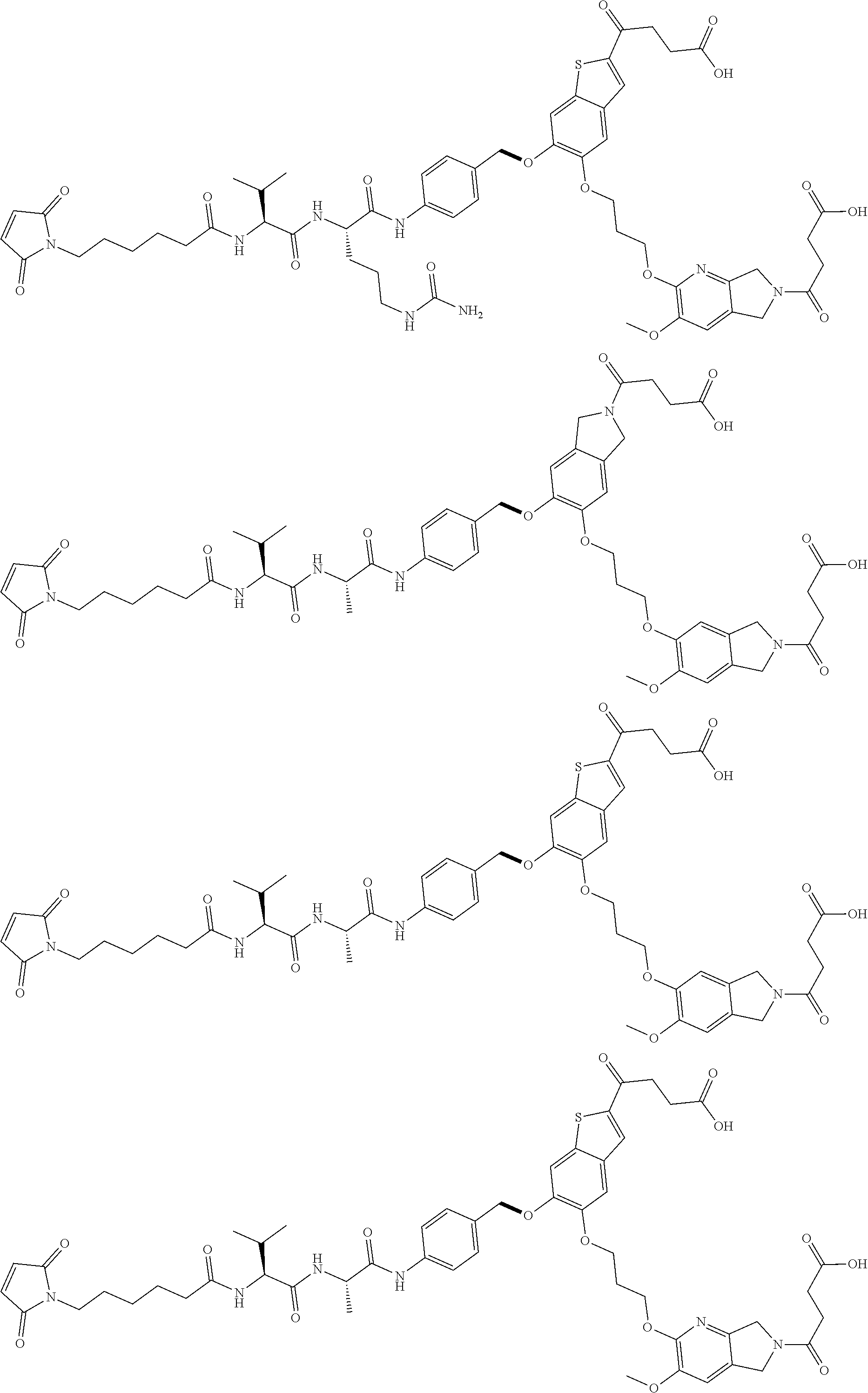

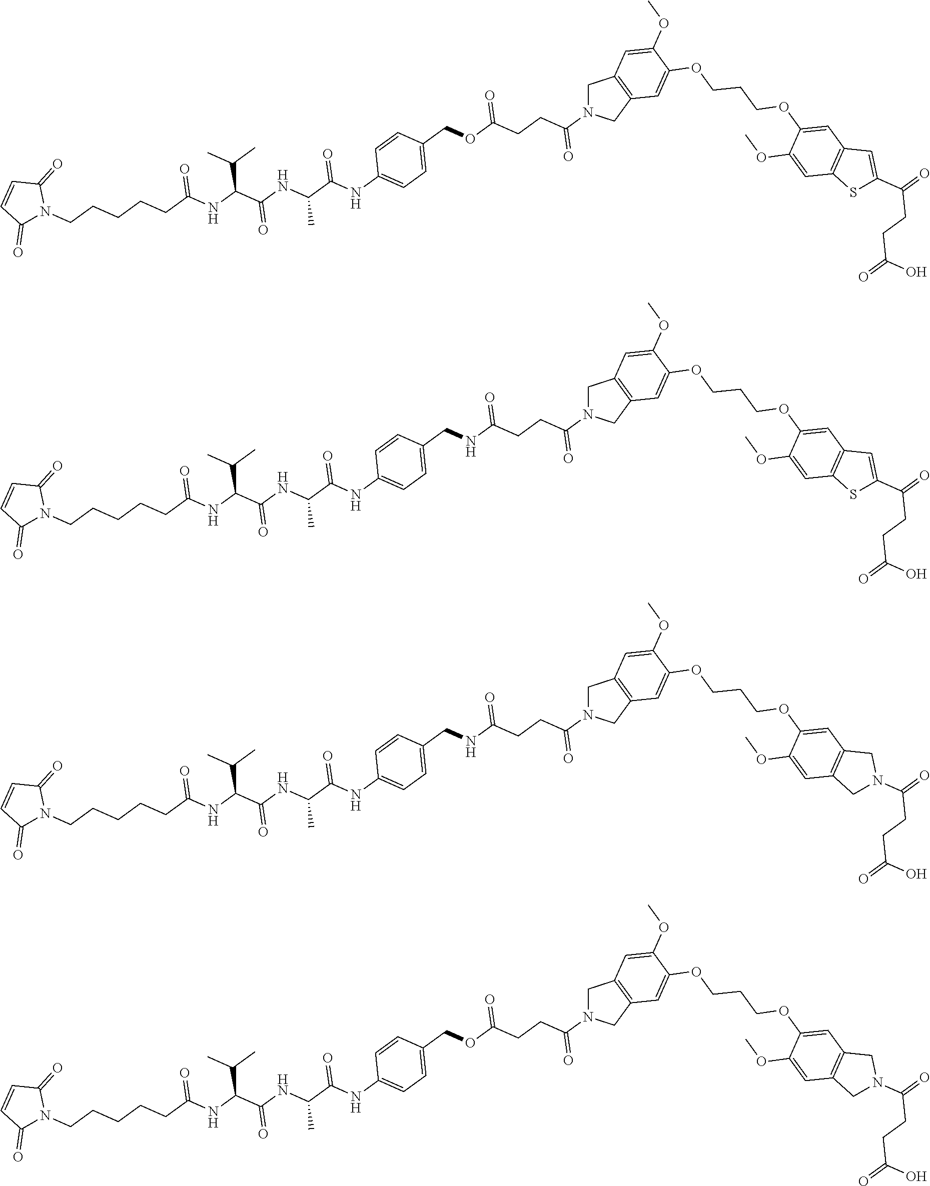

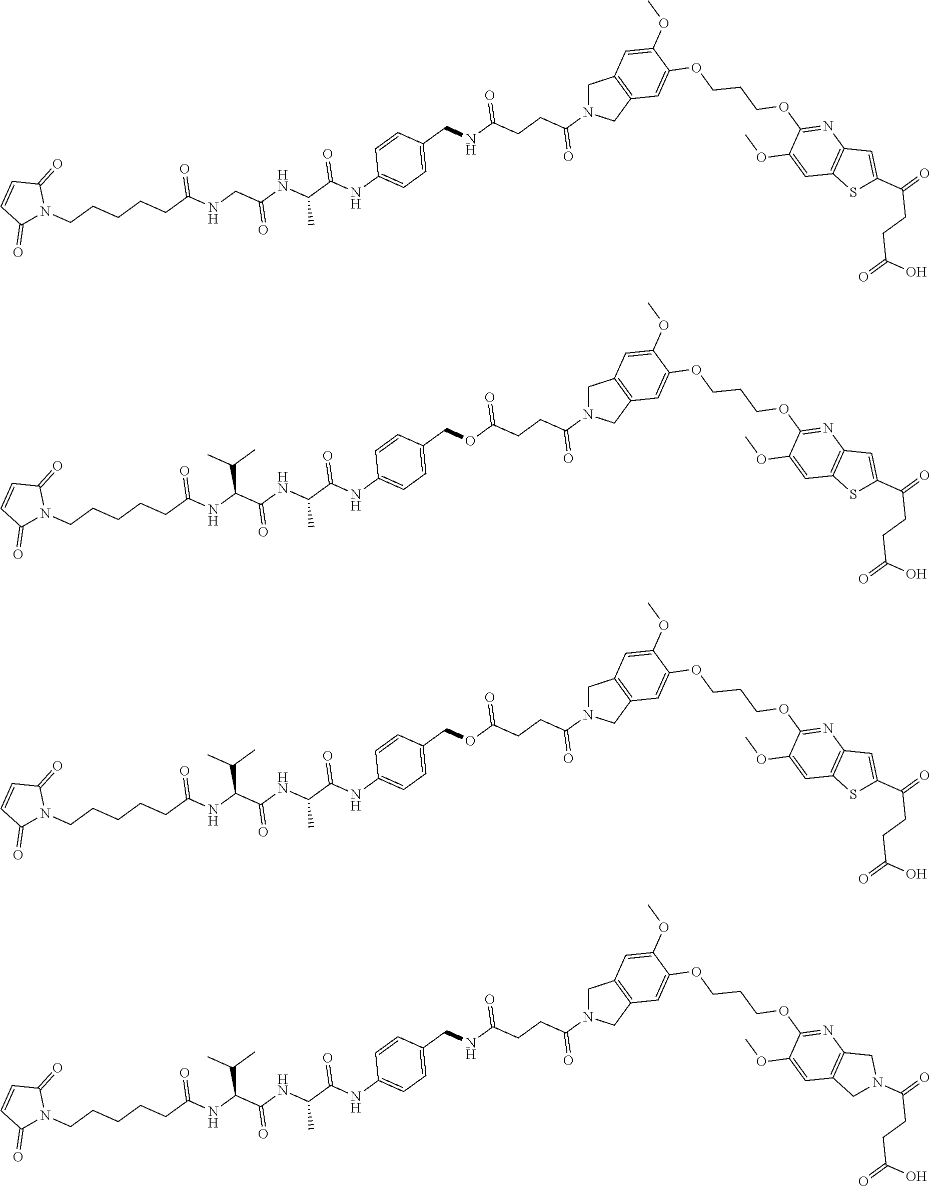

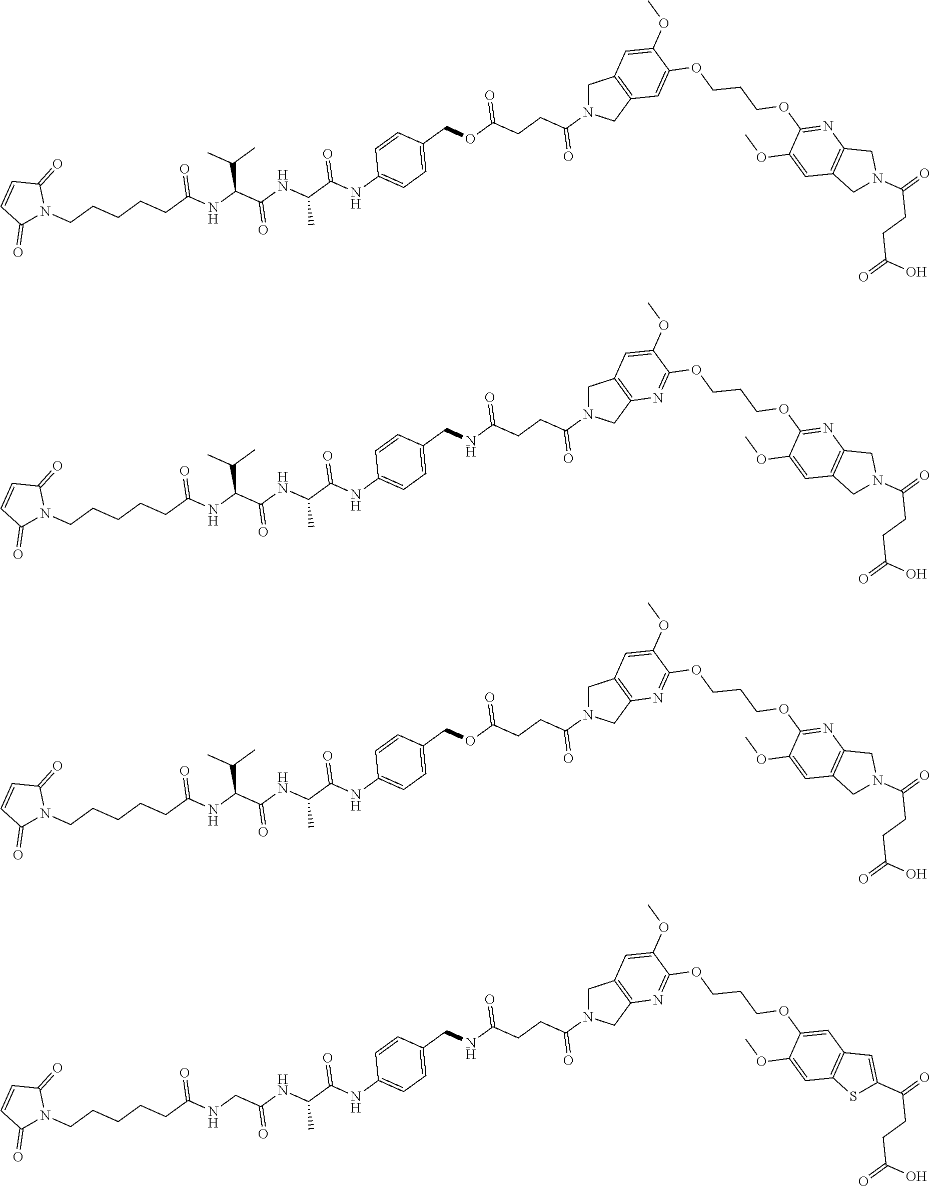

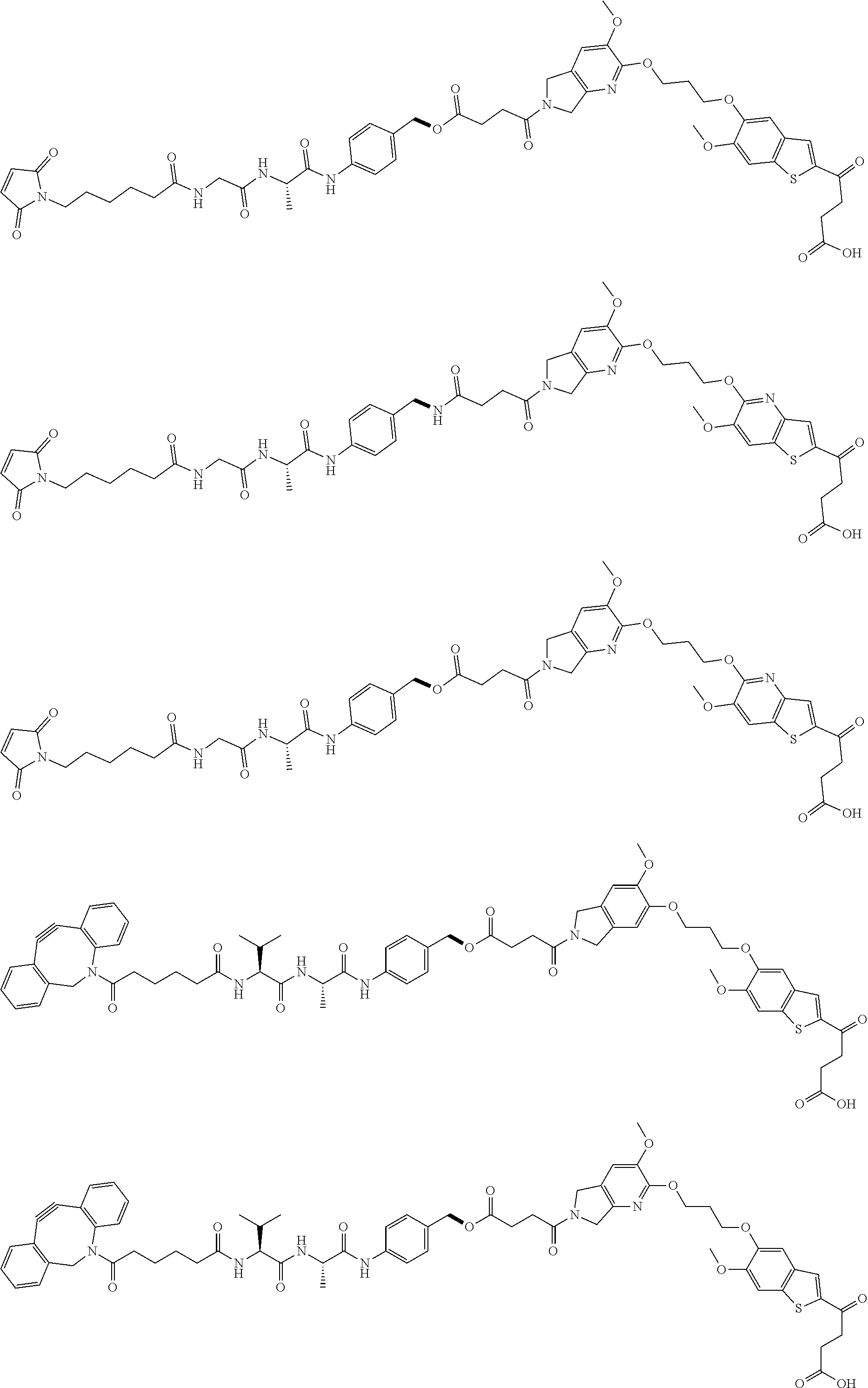

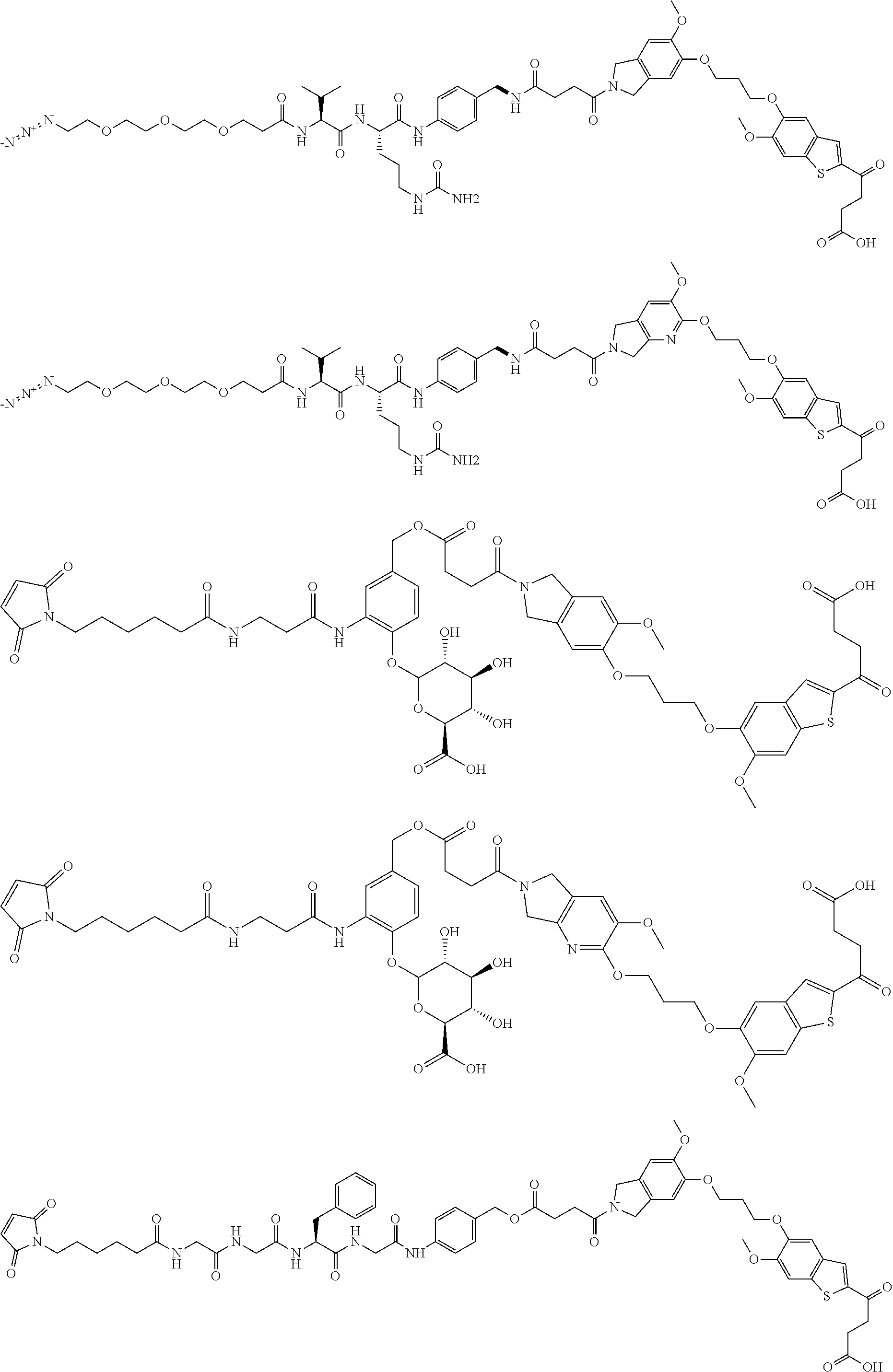

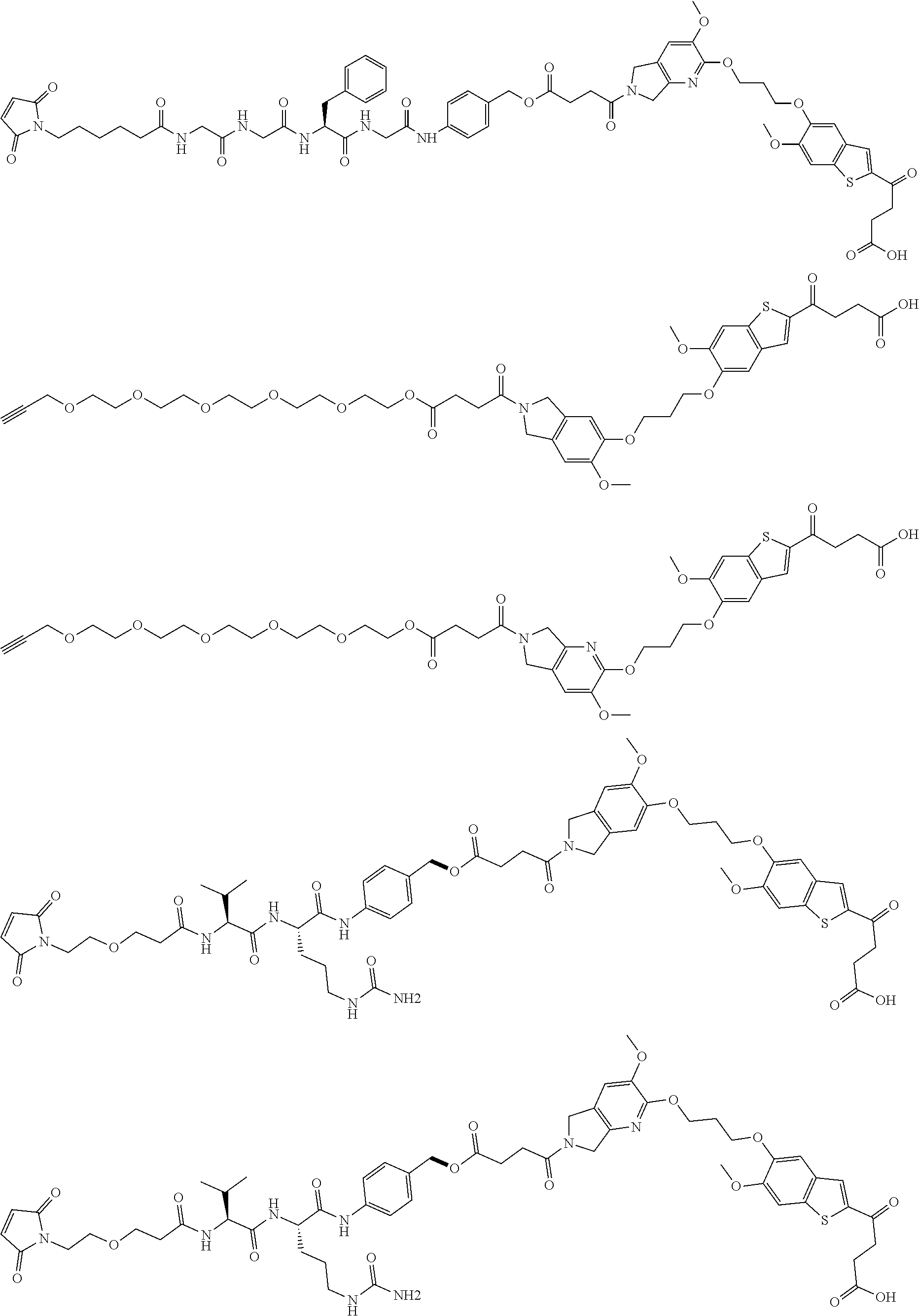

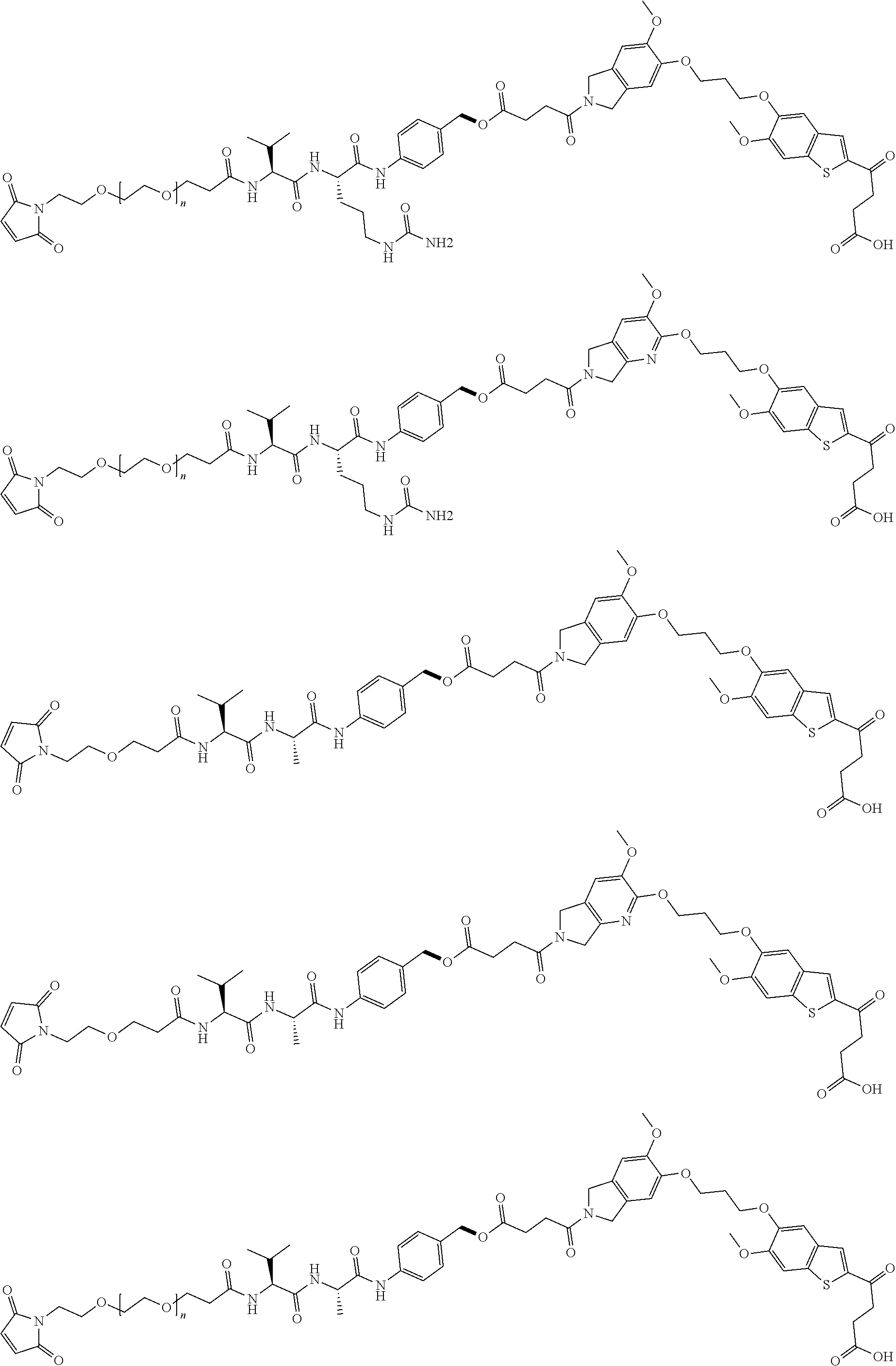

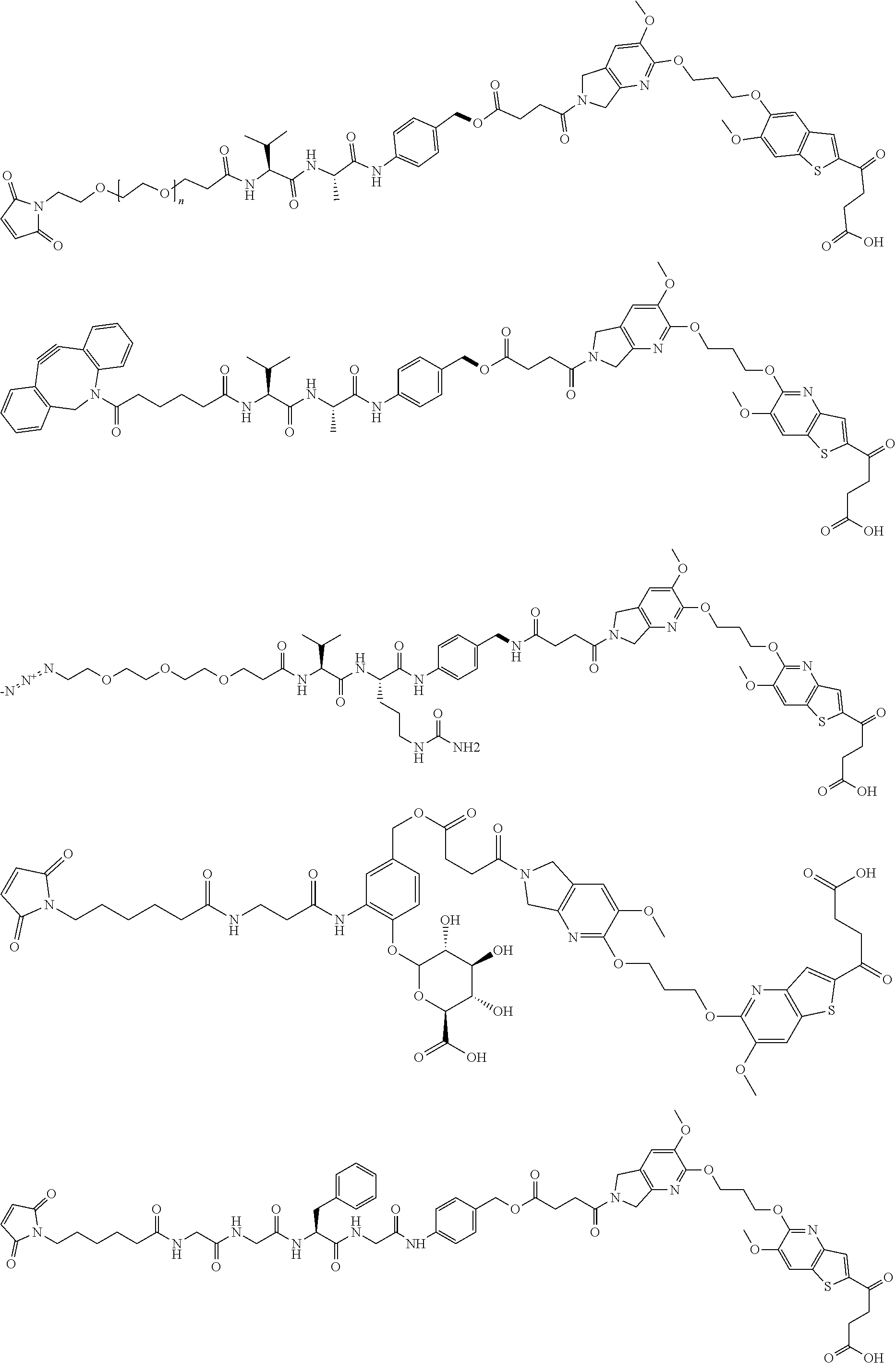

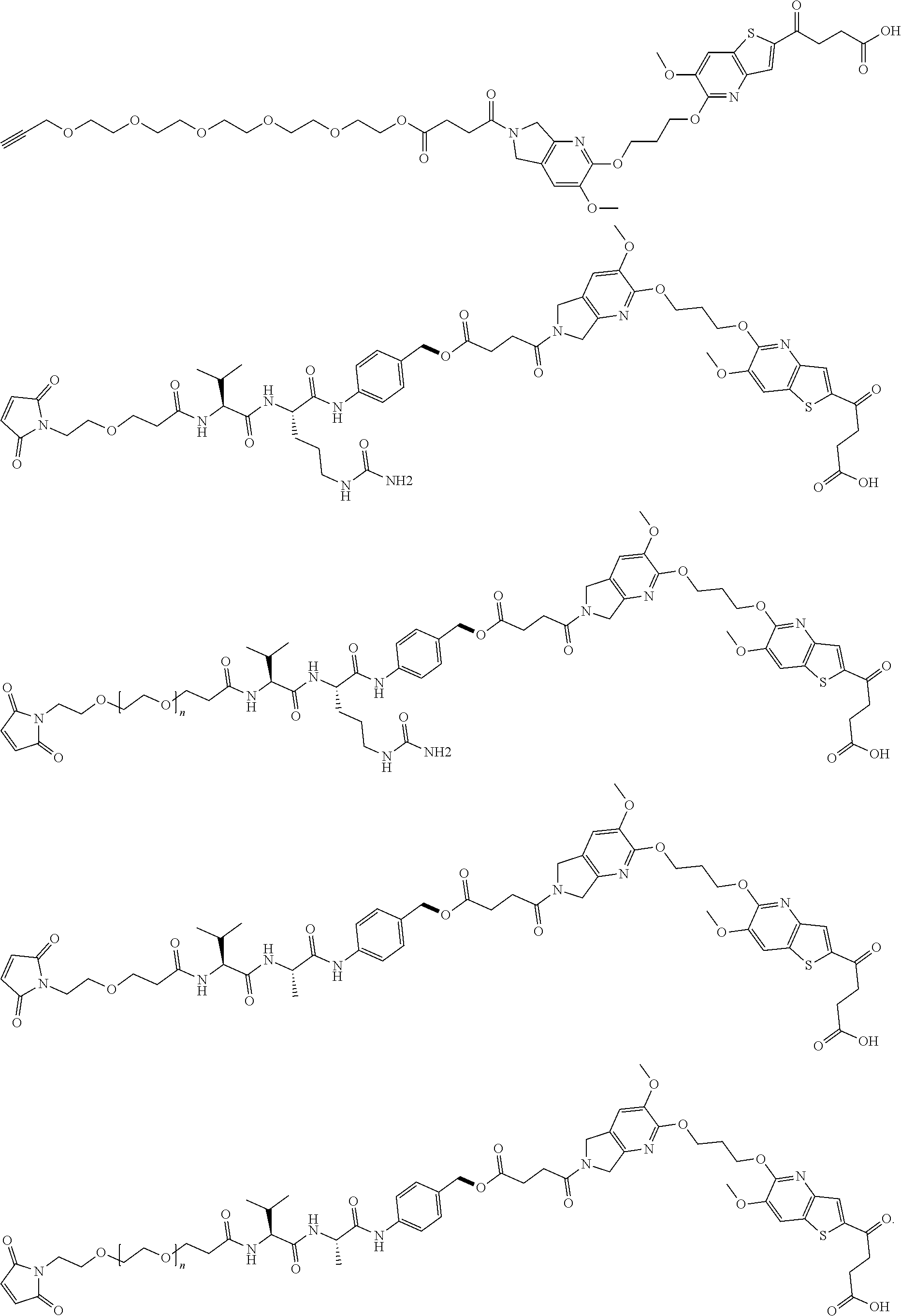

53. The compound-linker construct or conjugate of any one of the preceding claims, wherein the conjugate is selected from: 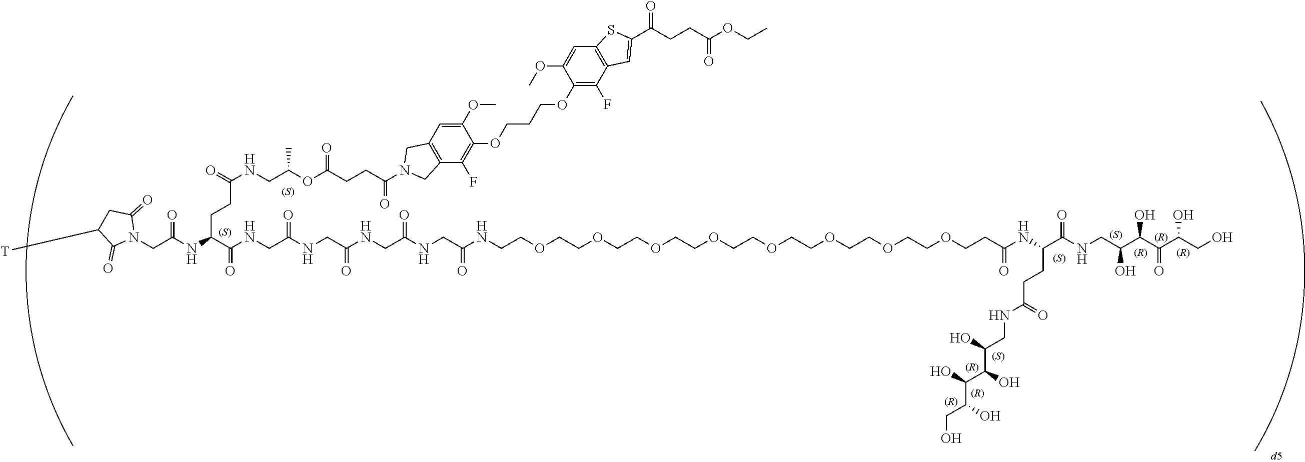 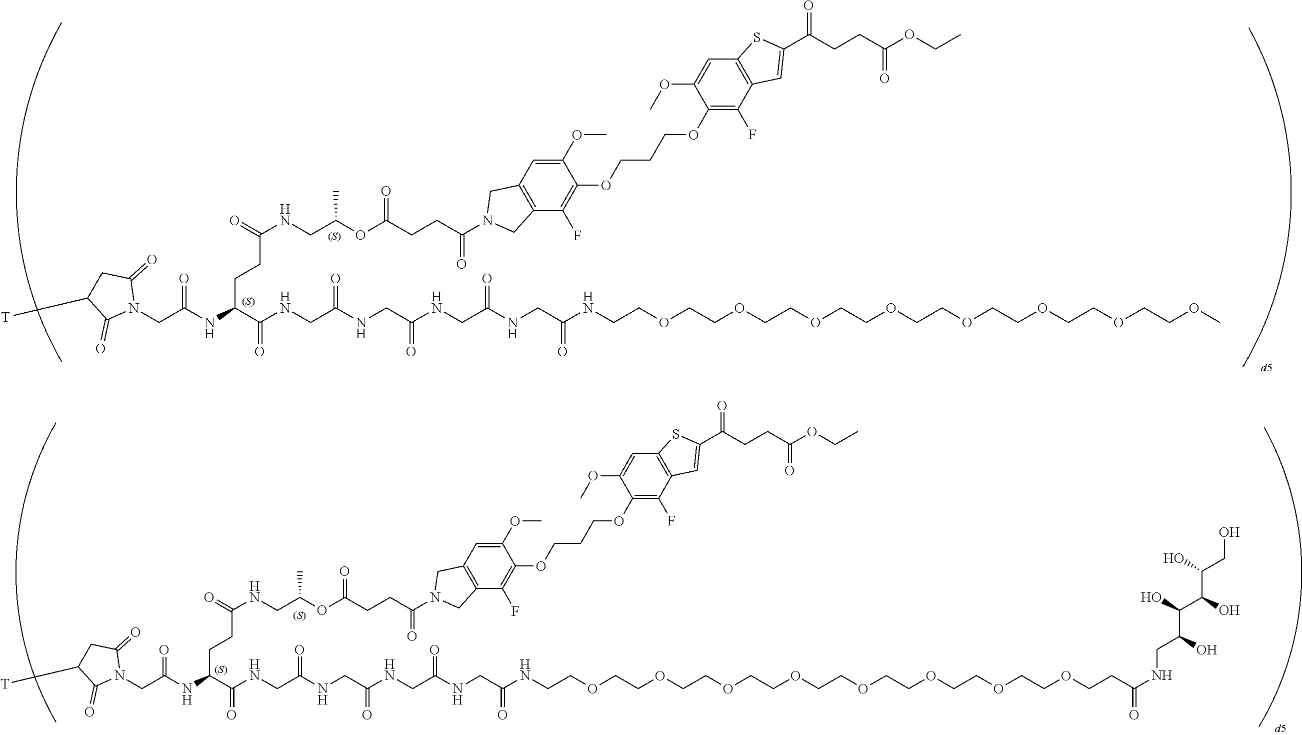 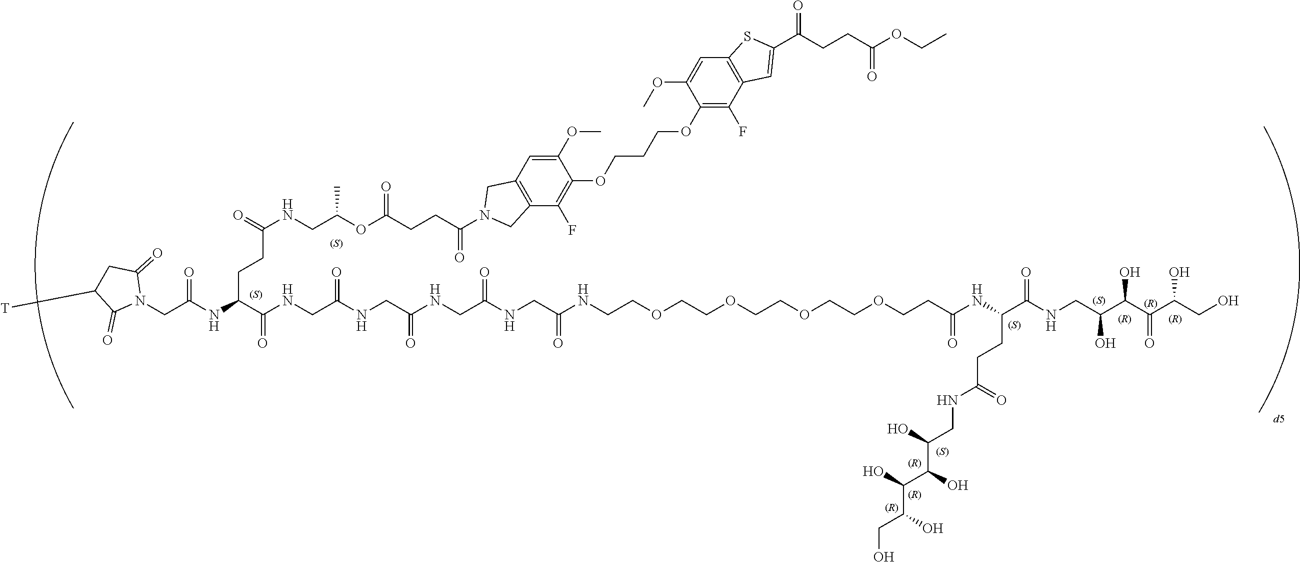 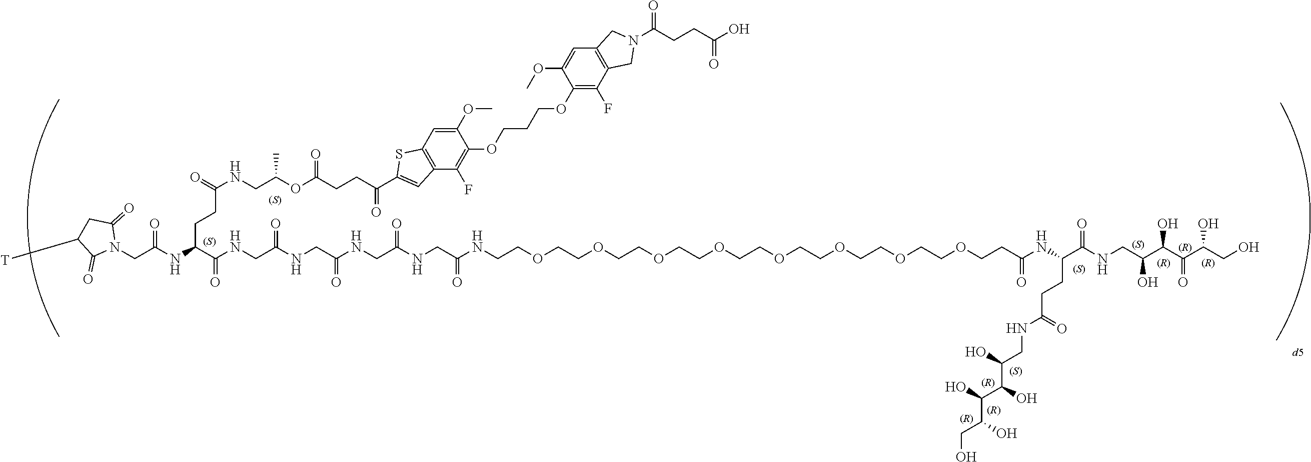 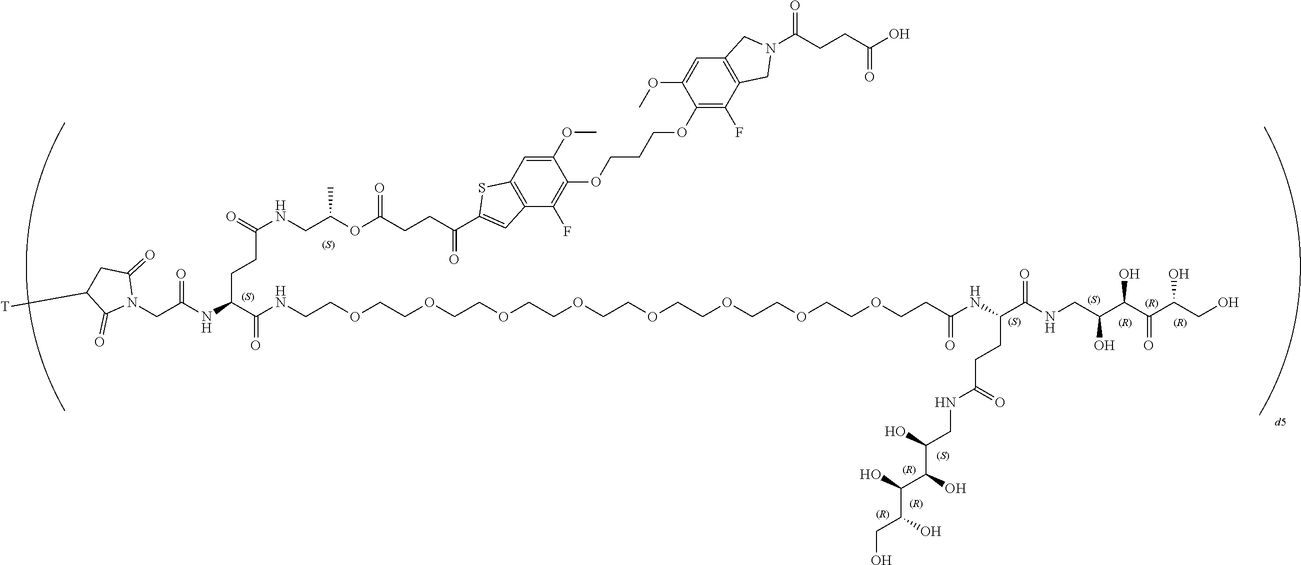 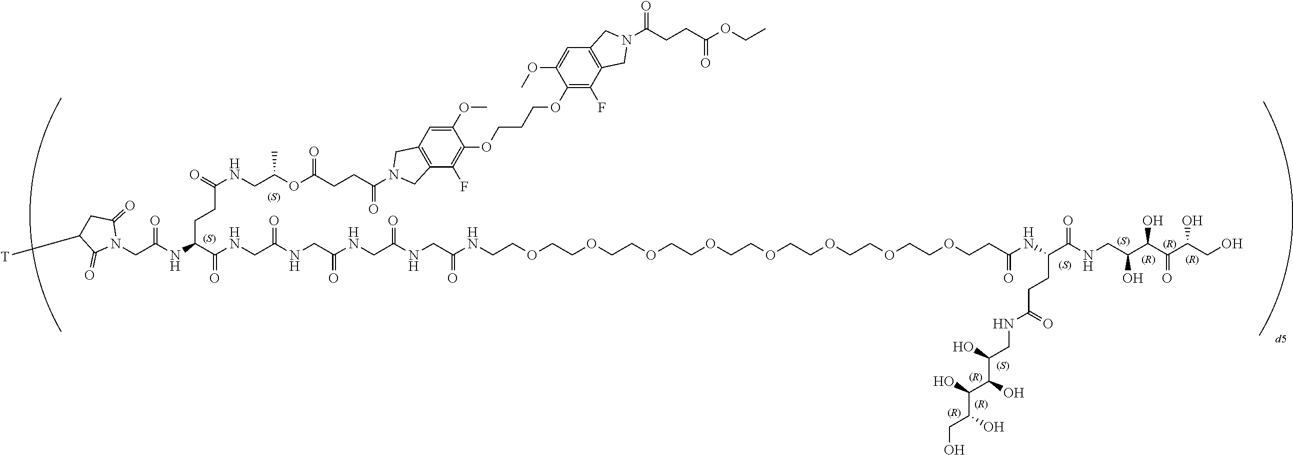 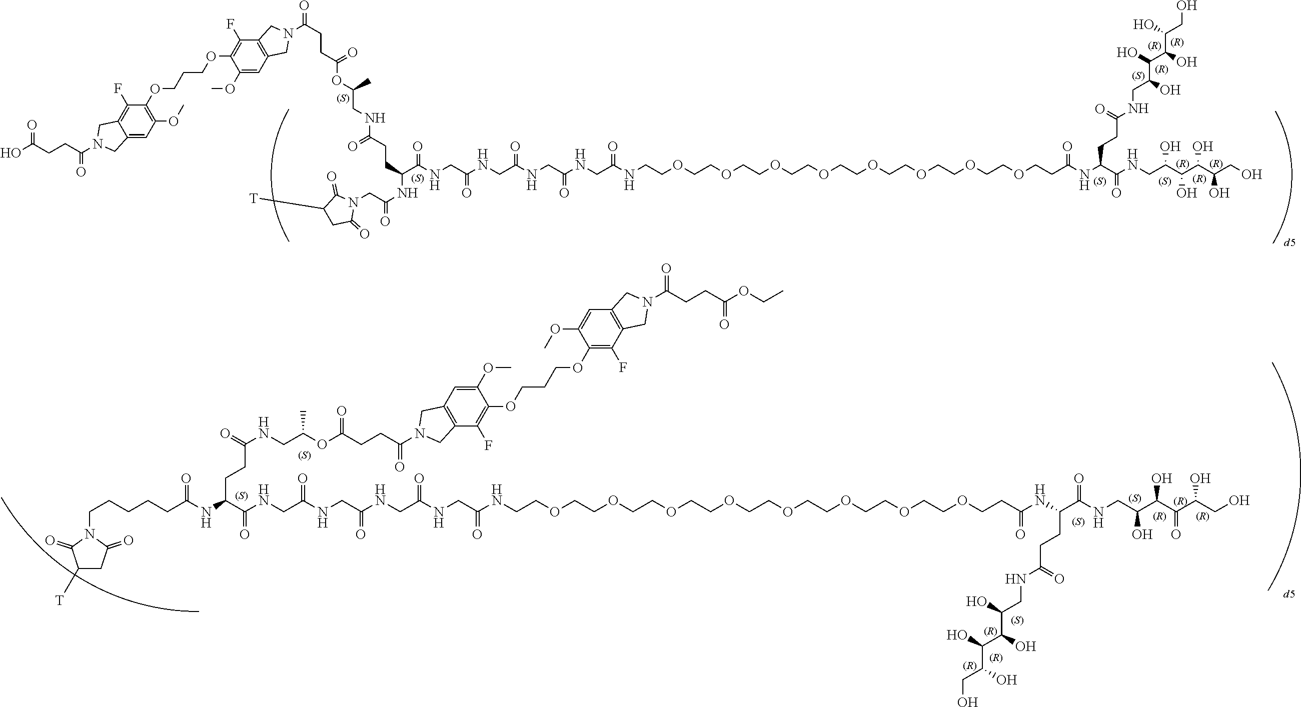 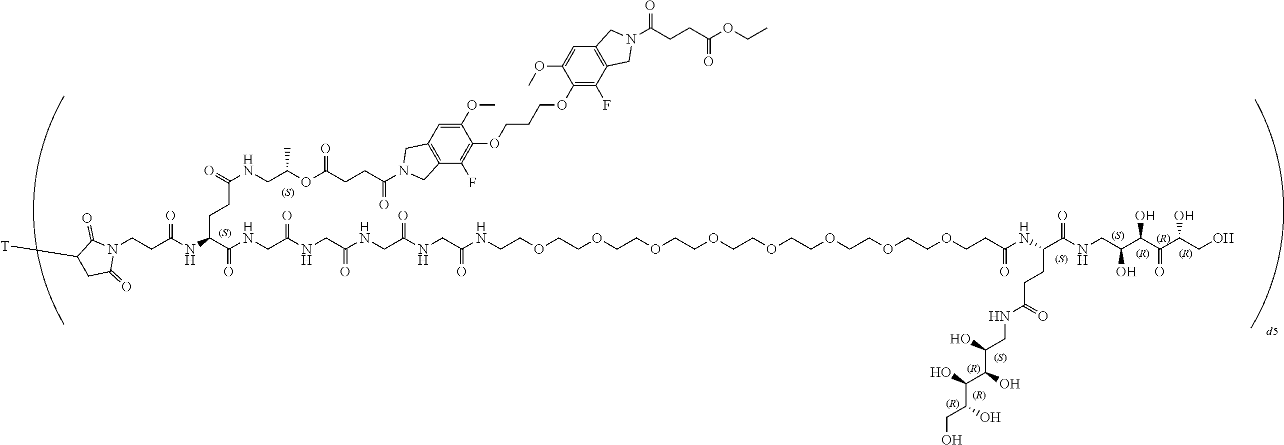 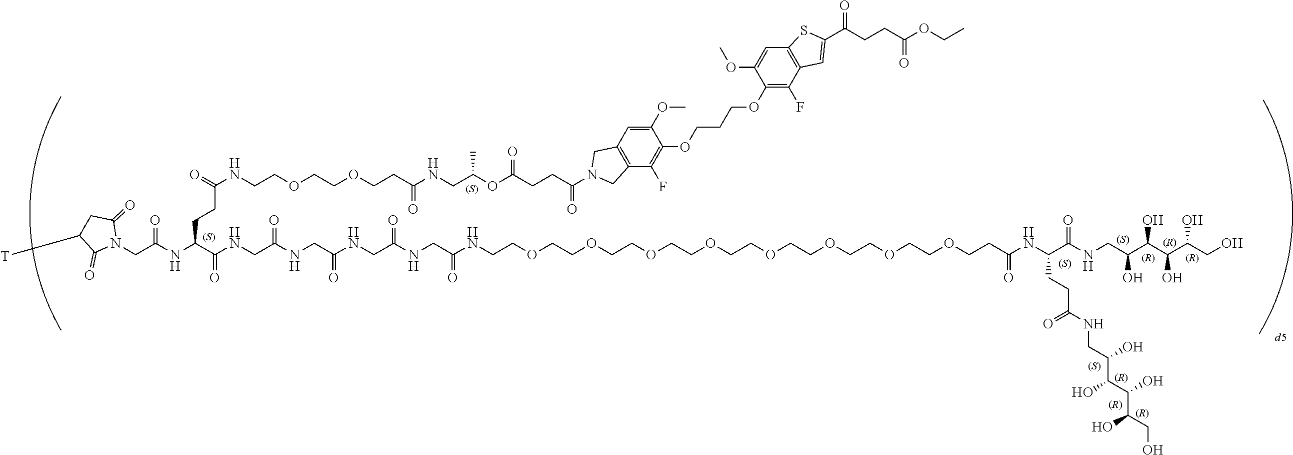 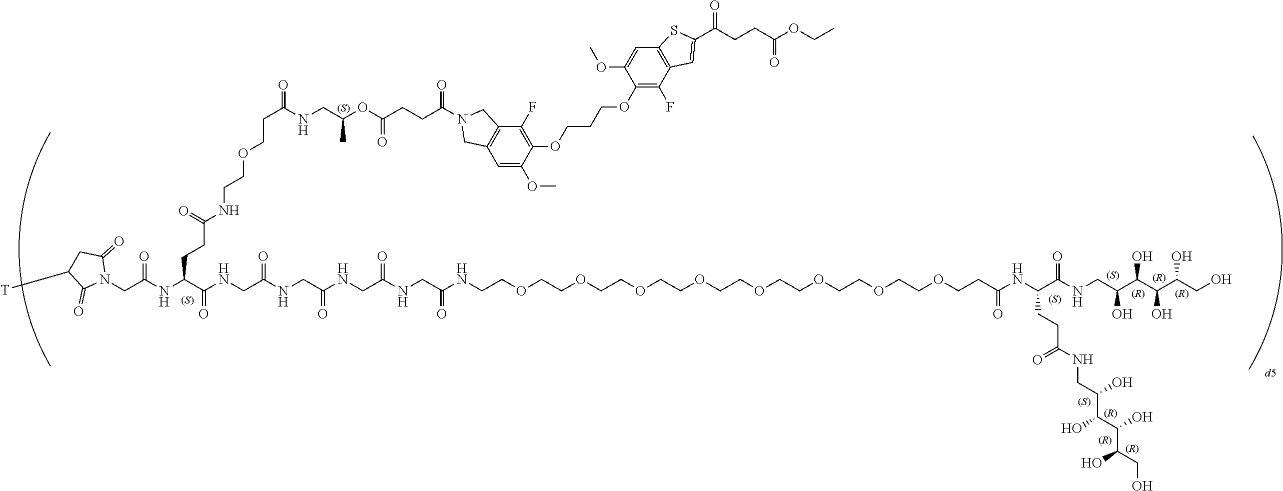 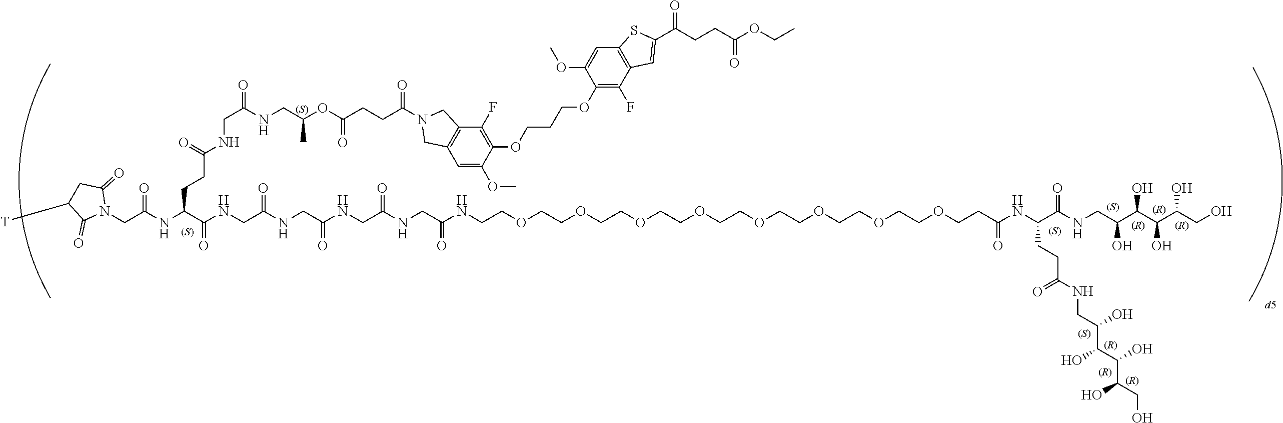 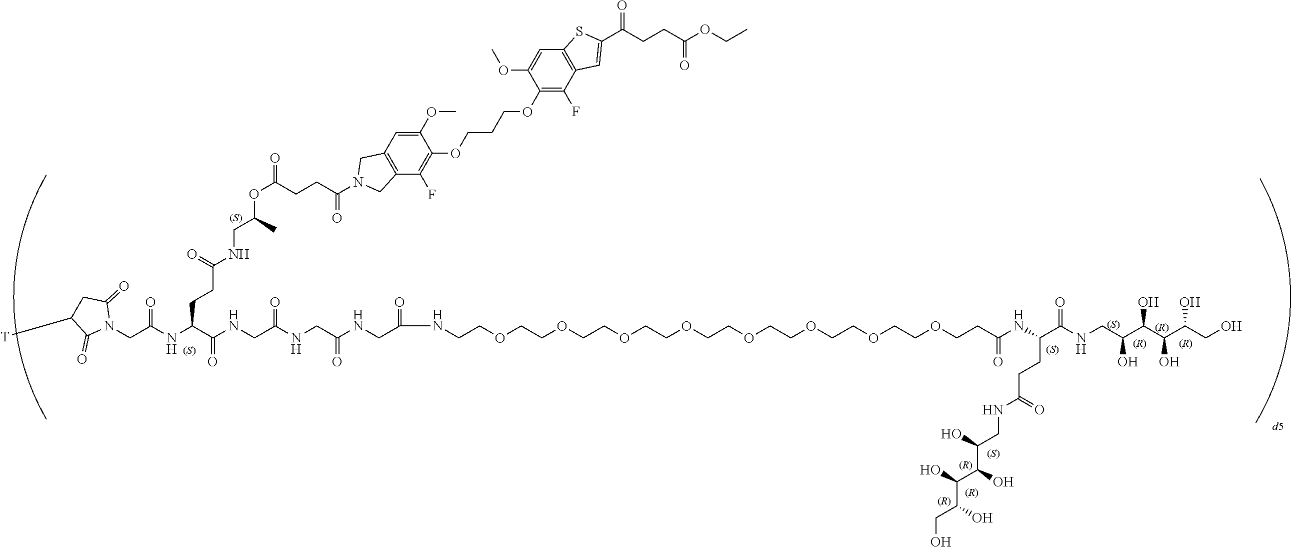 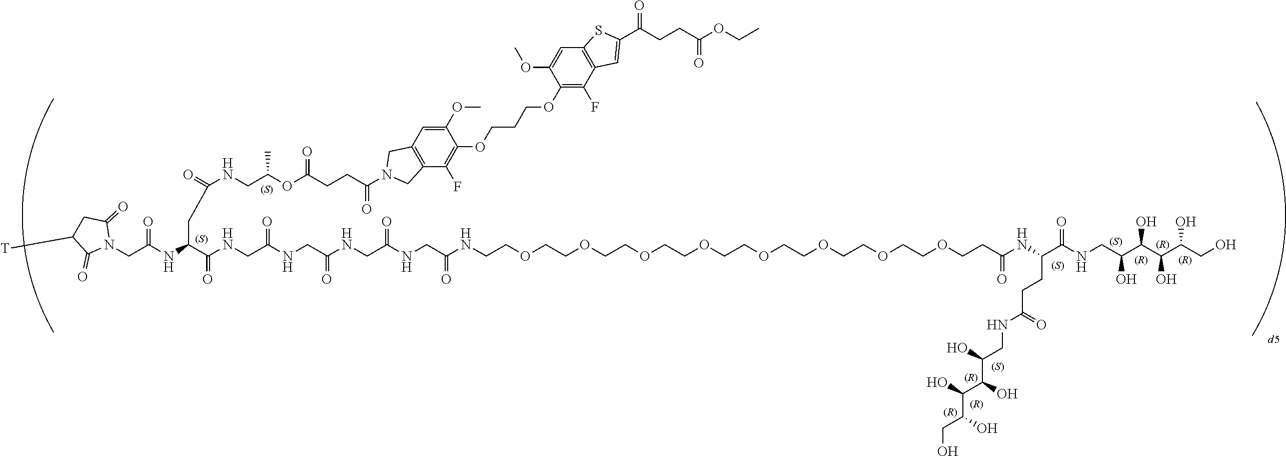 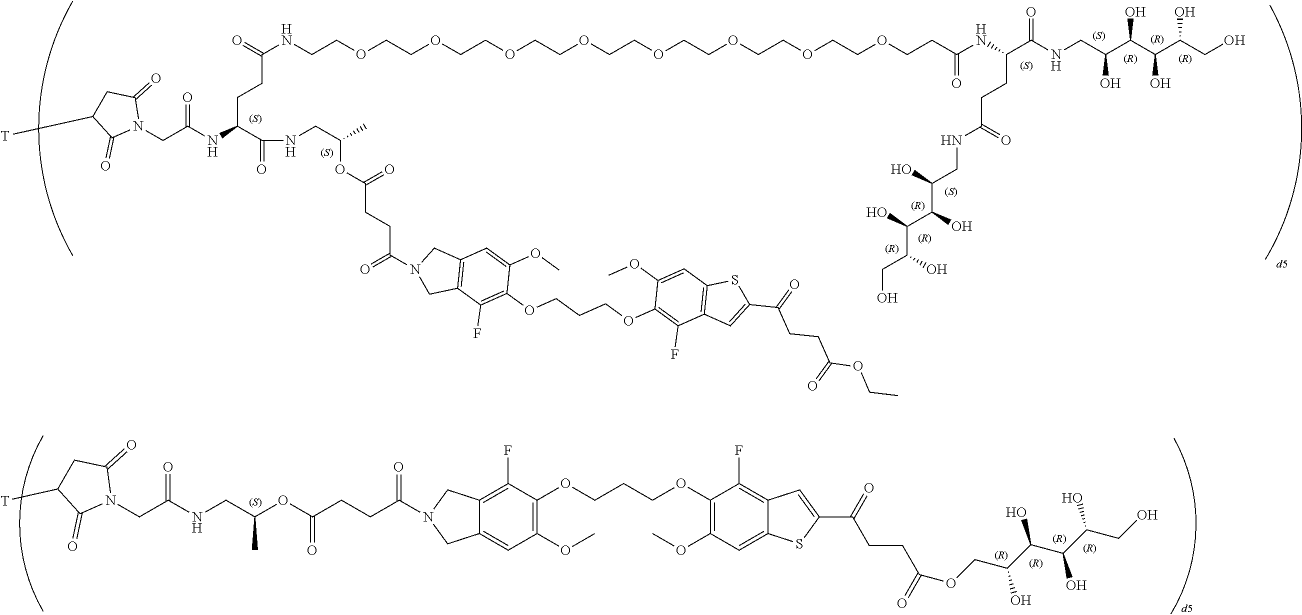 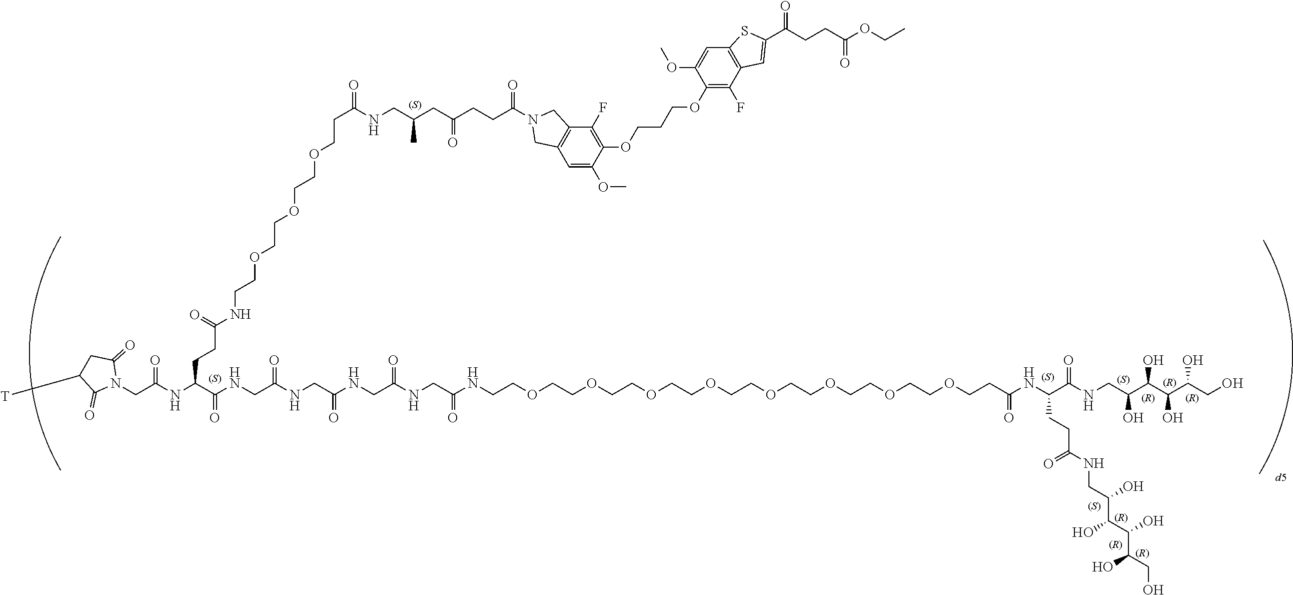 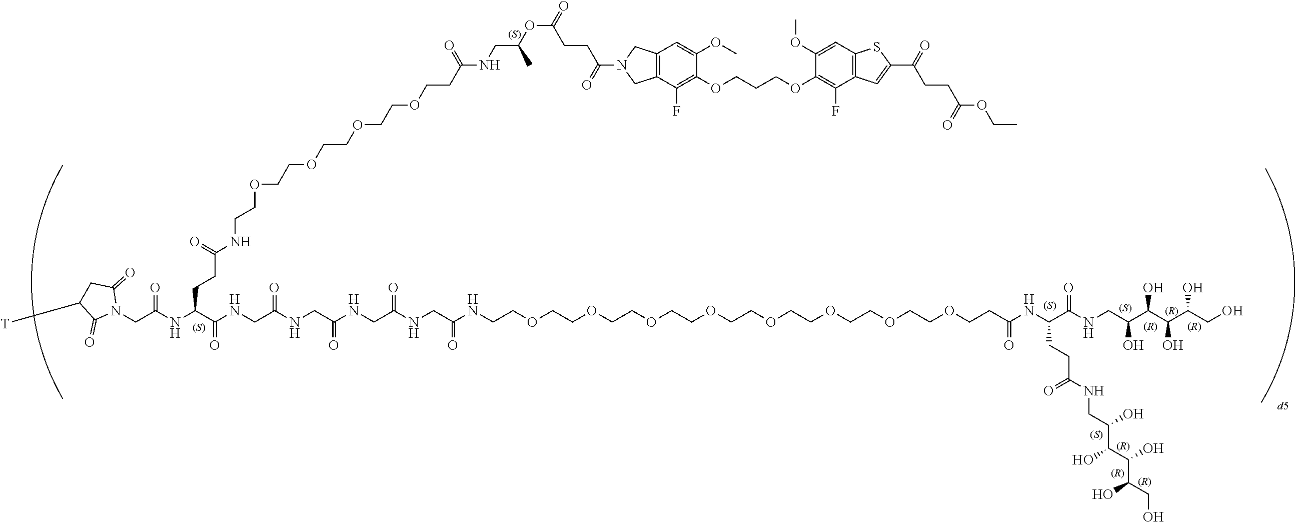 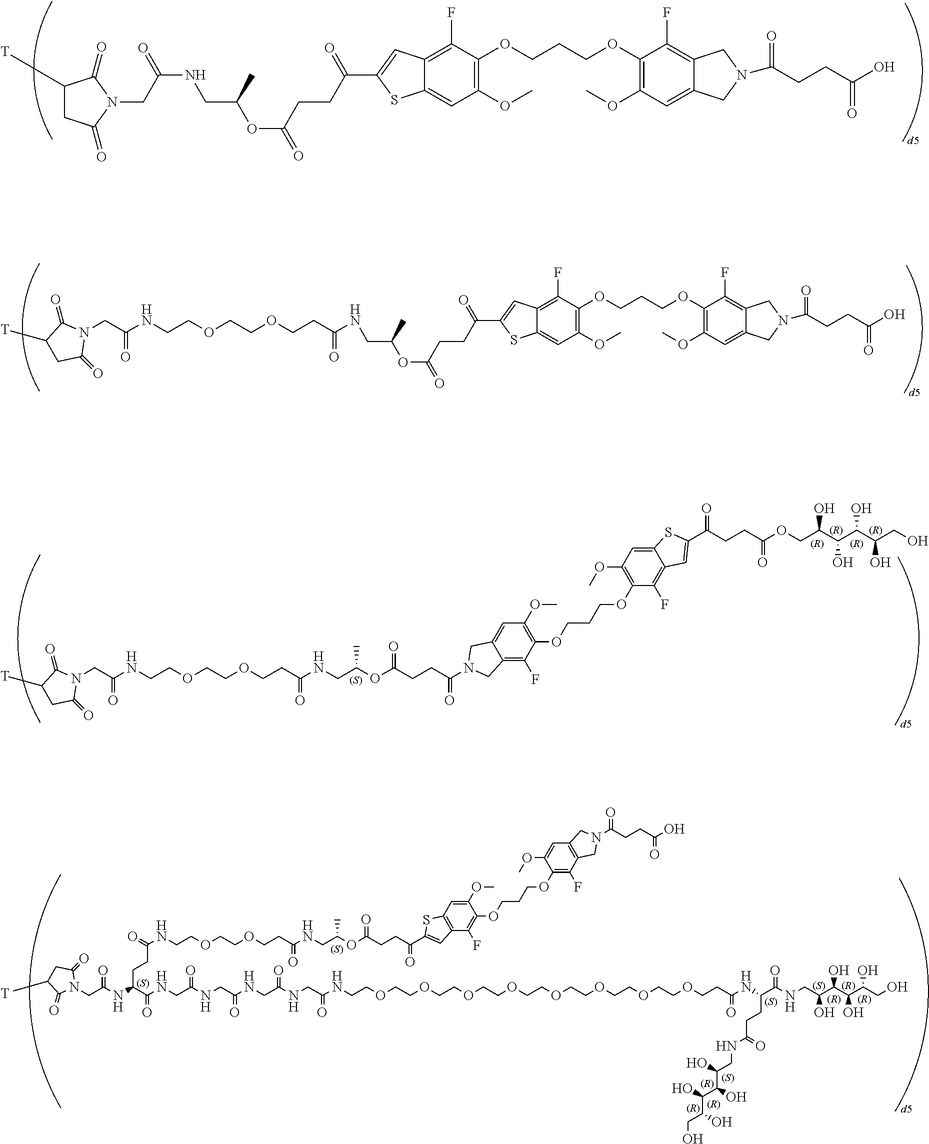 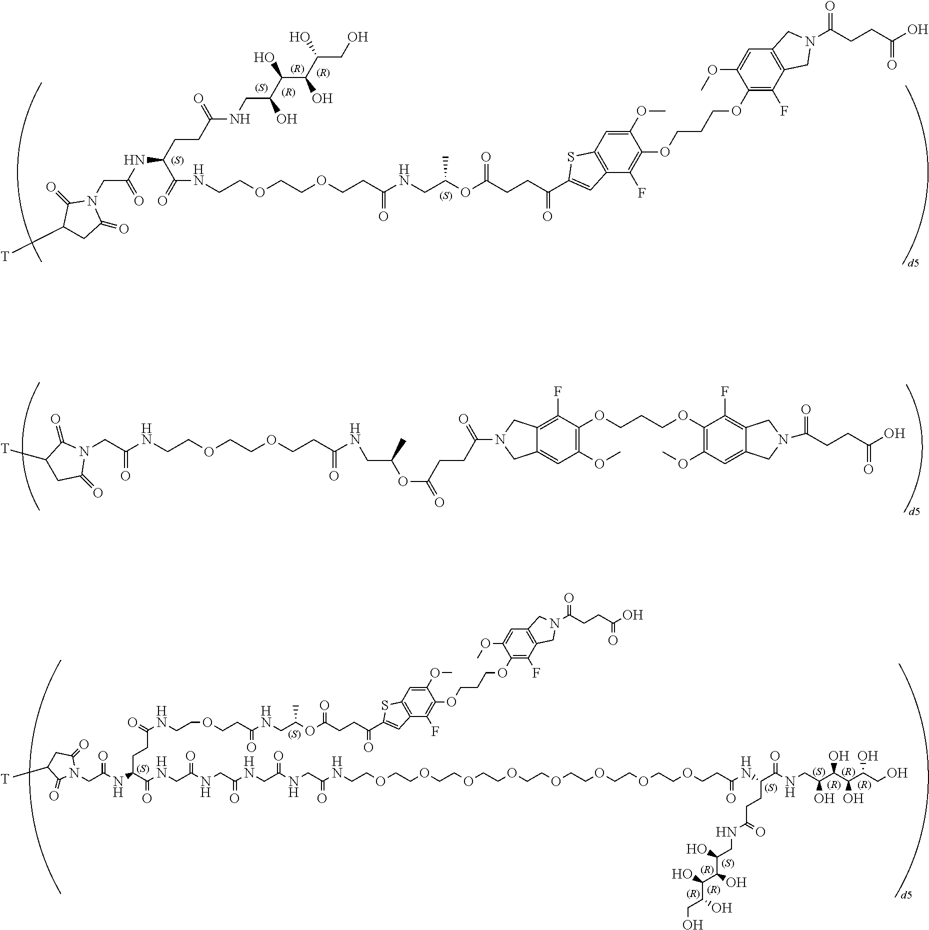 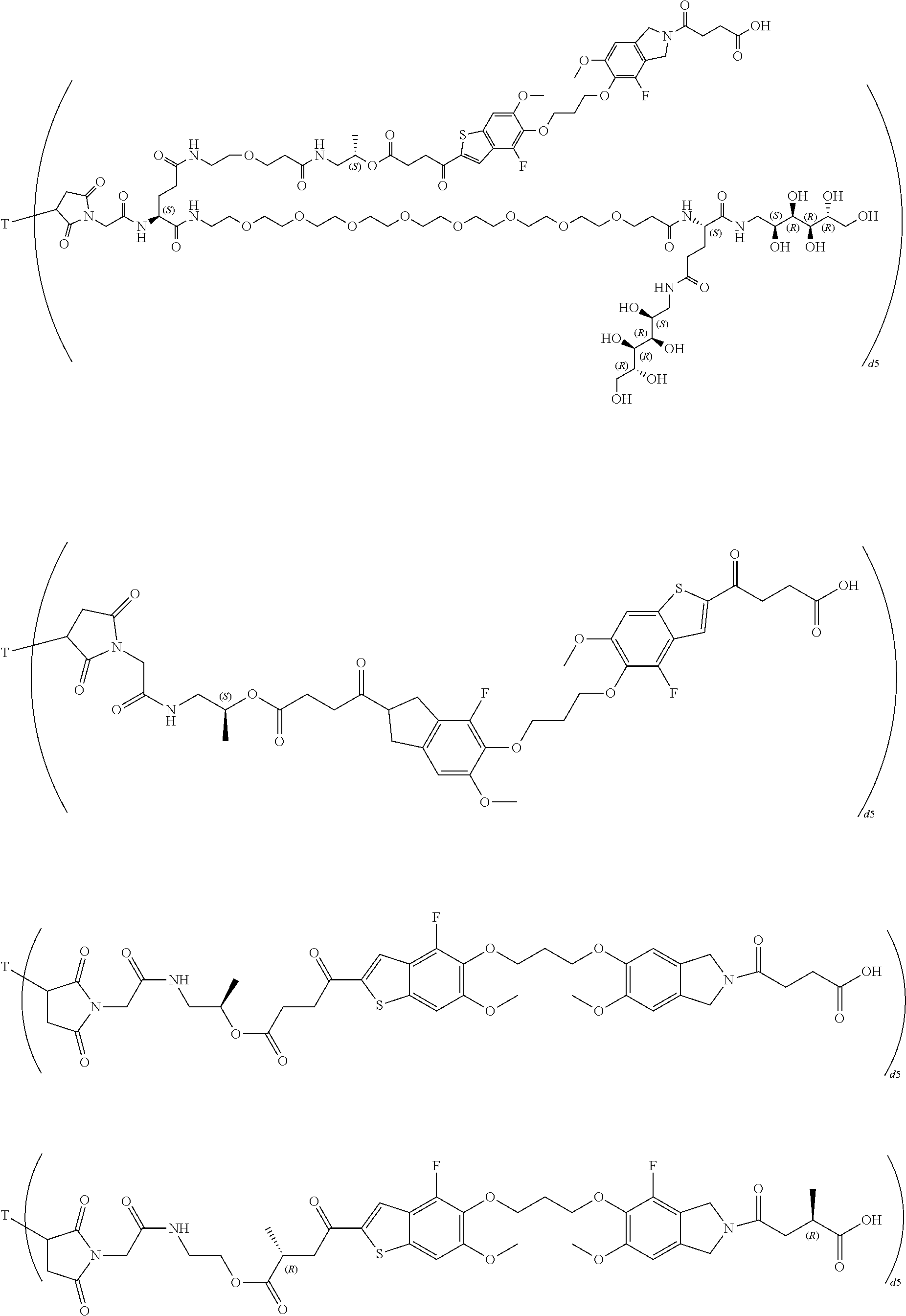 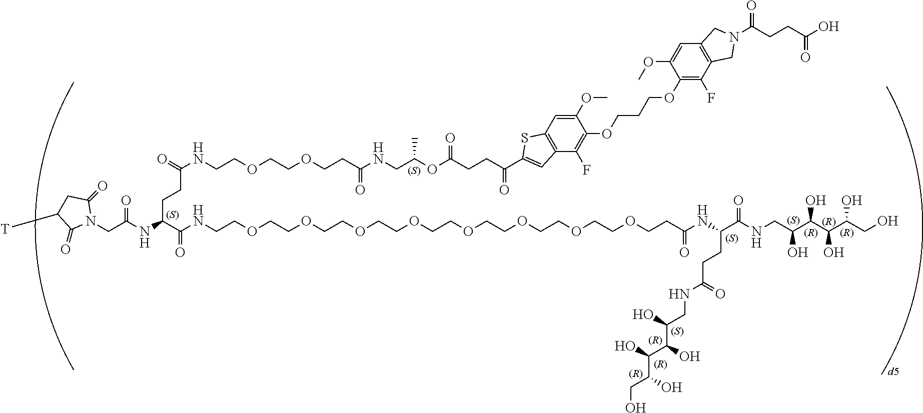 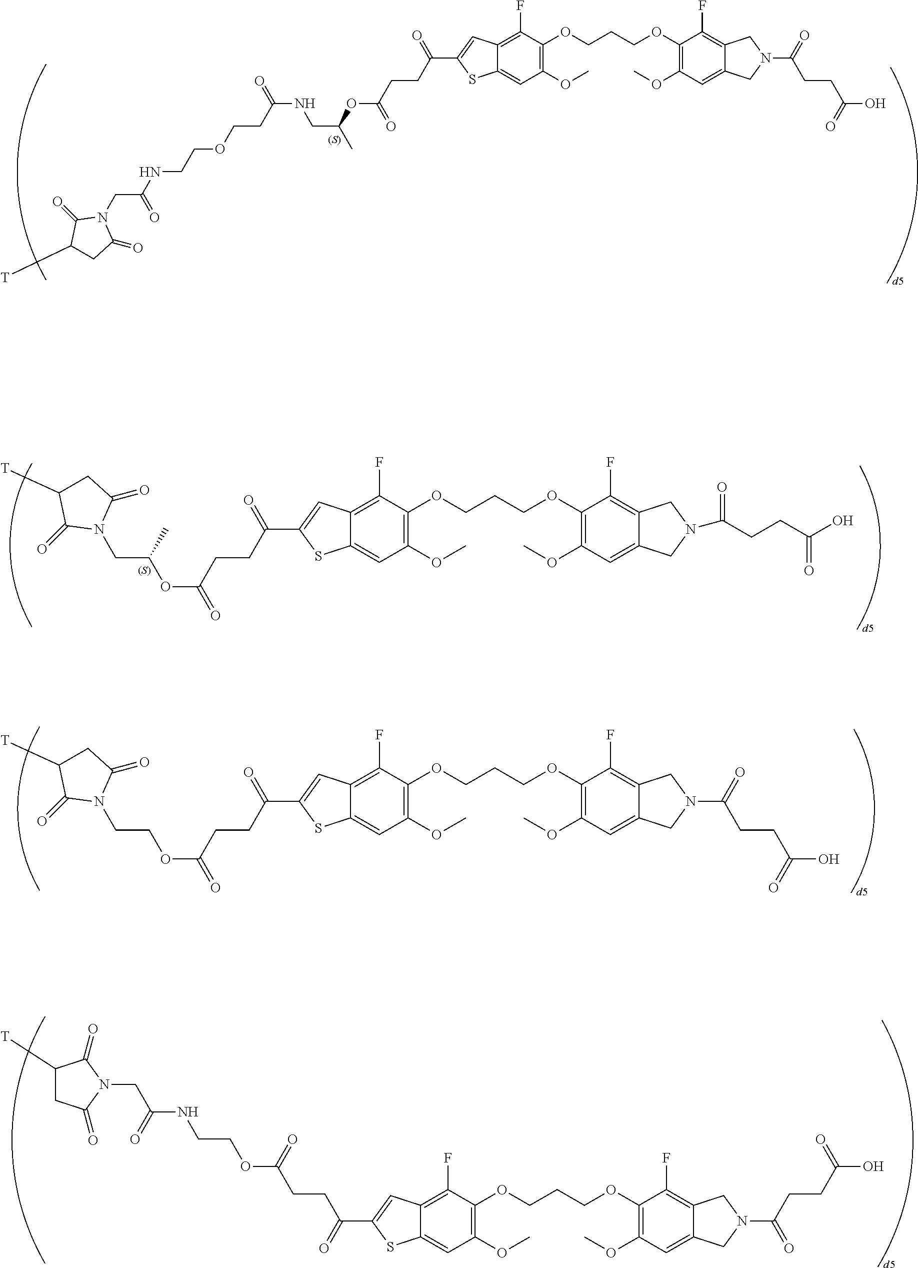 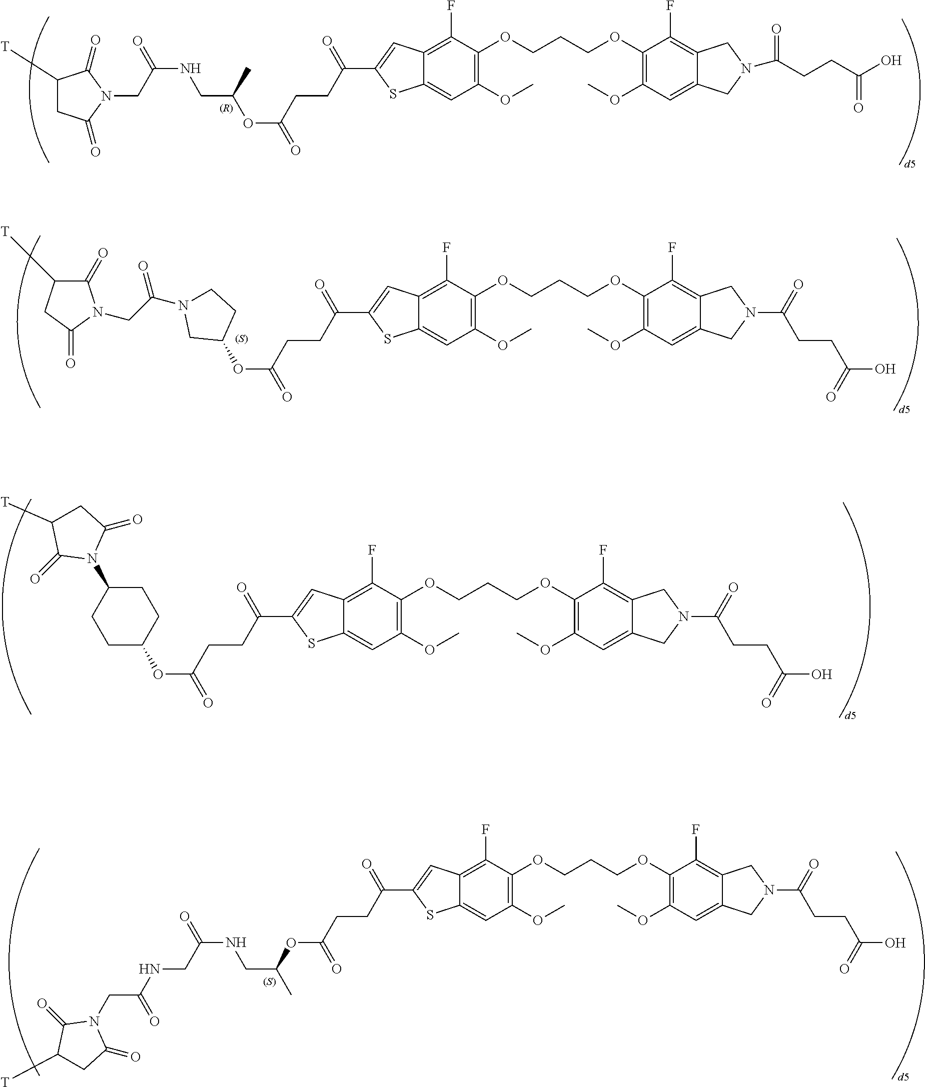 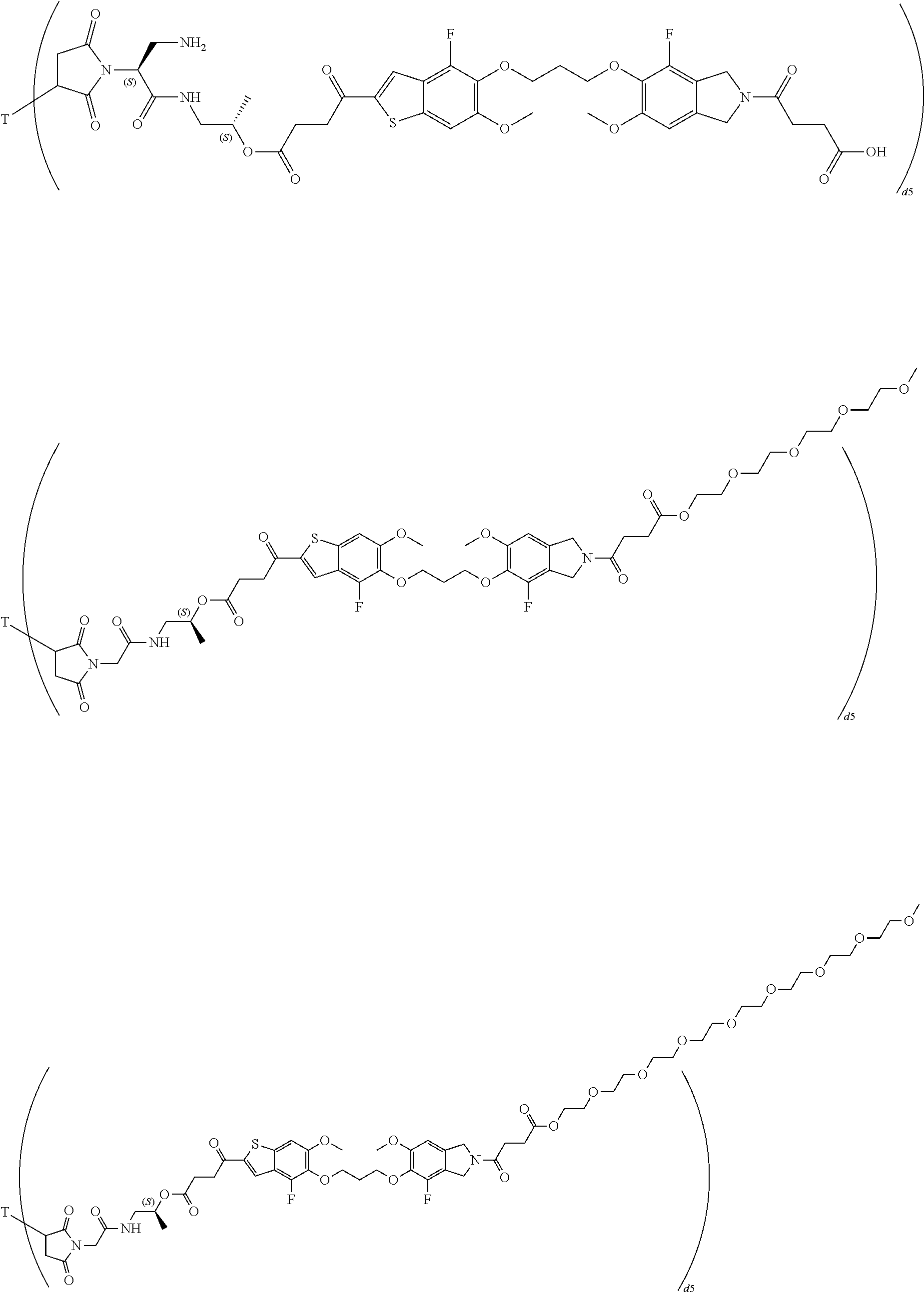 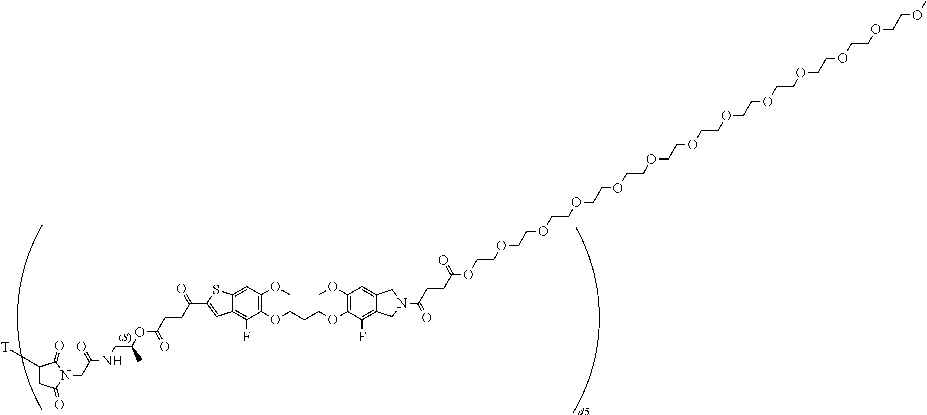 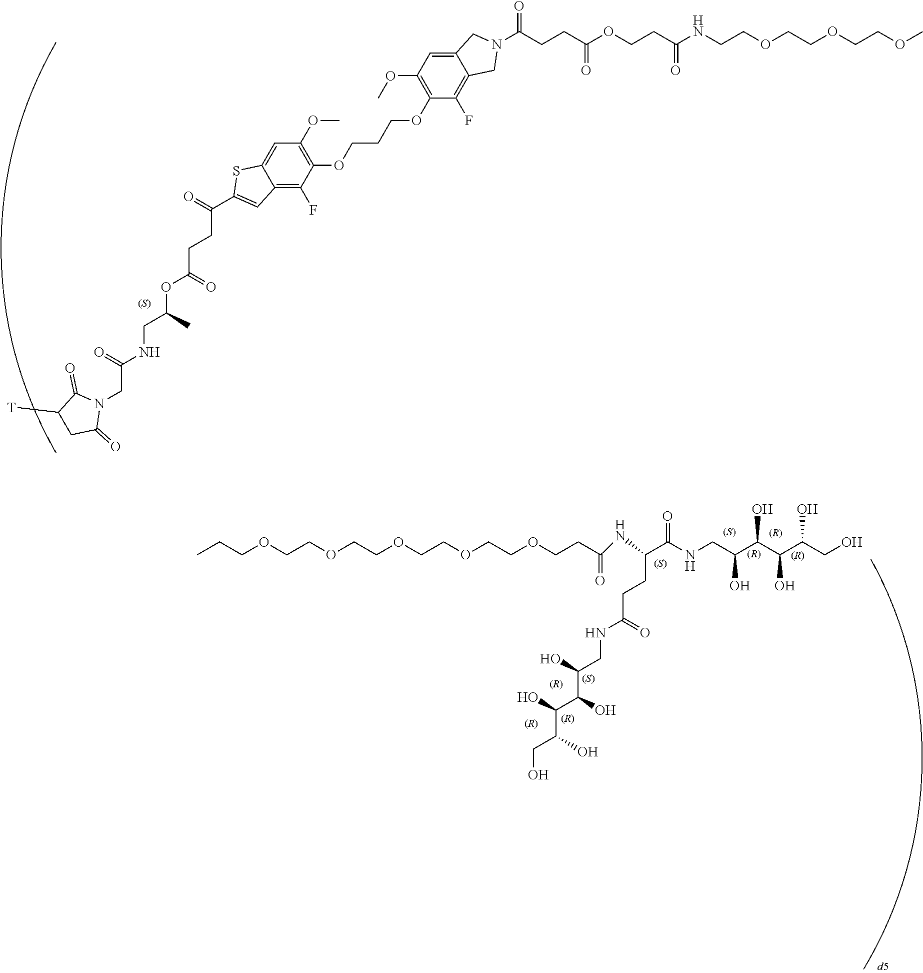 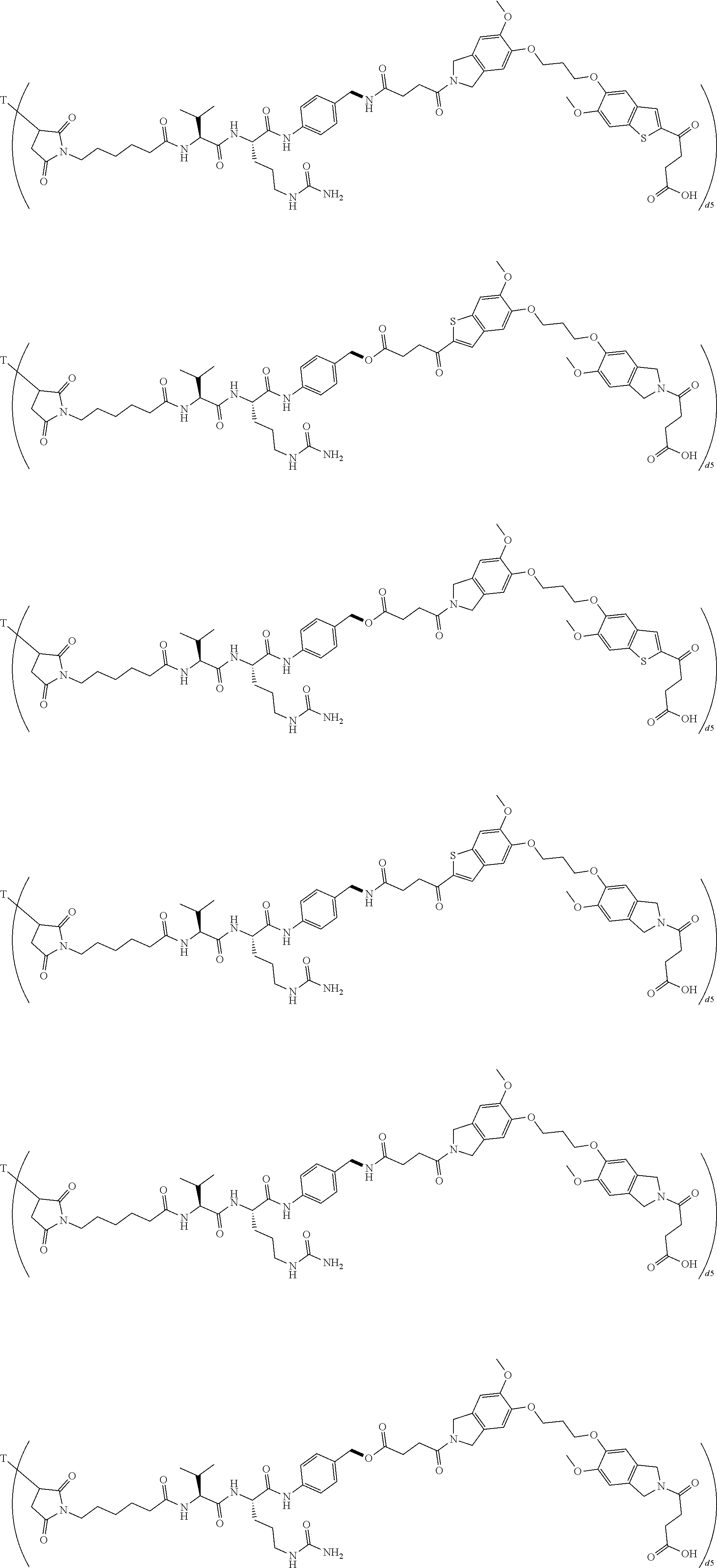 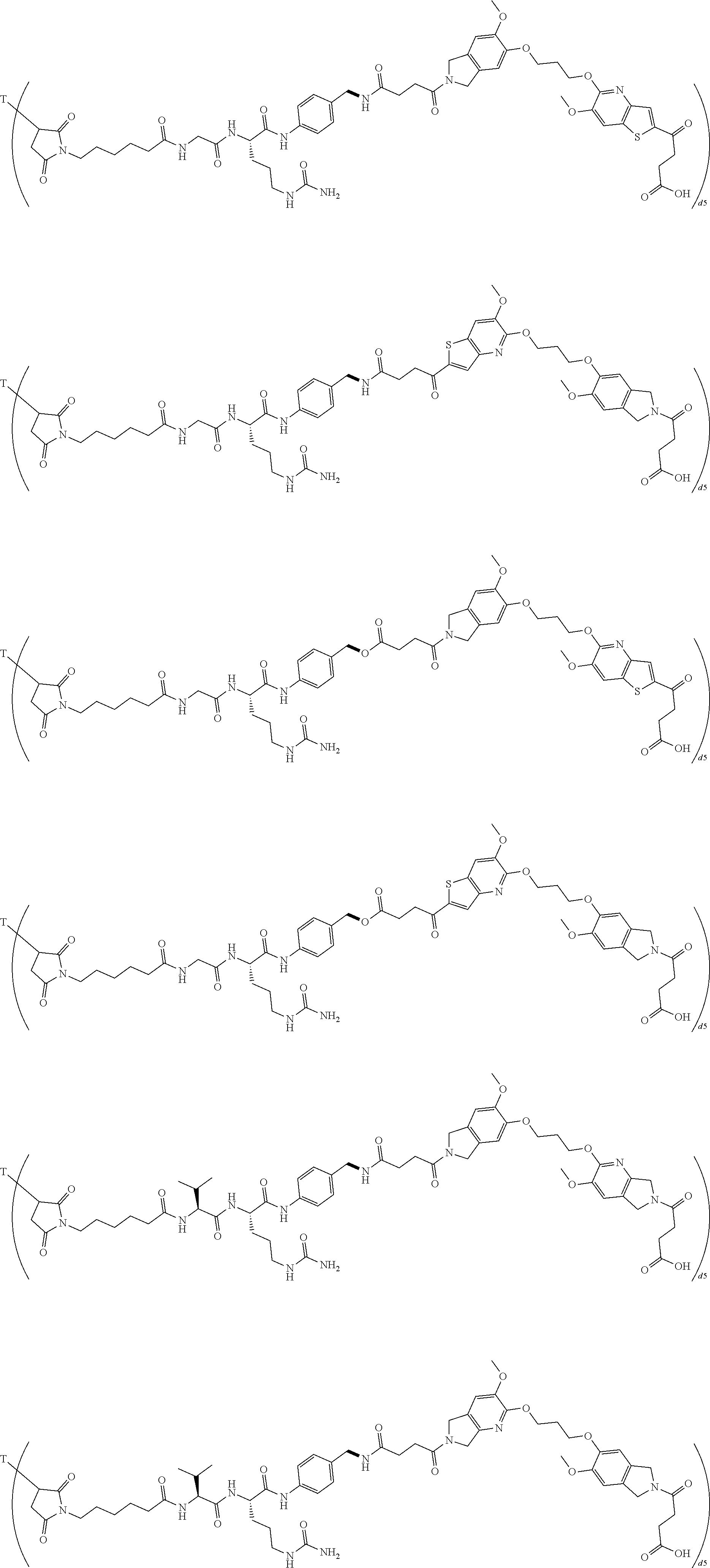 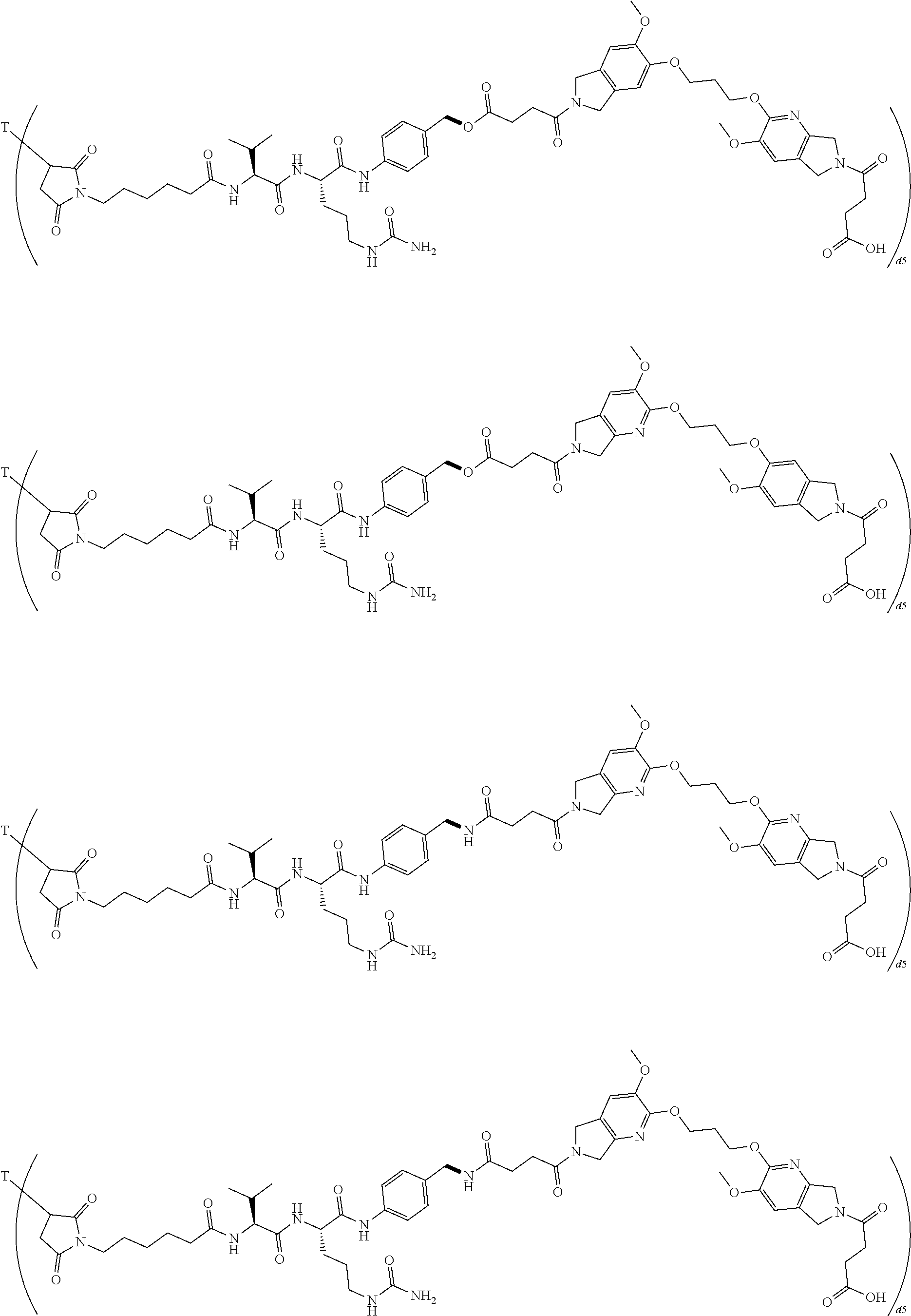 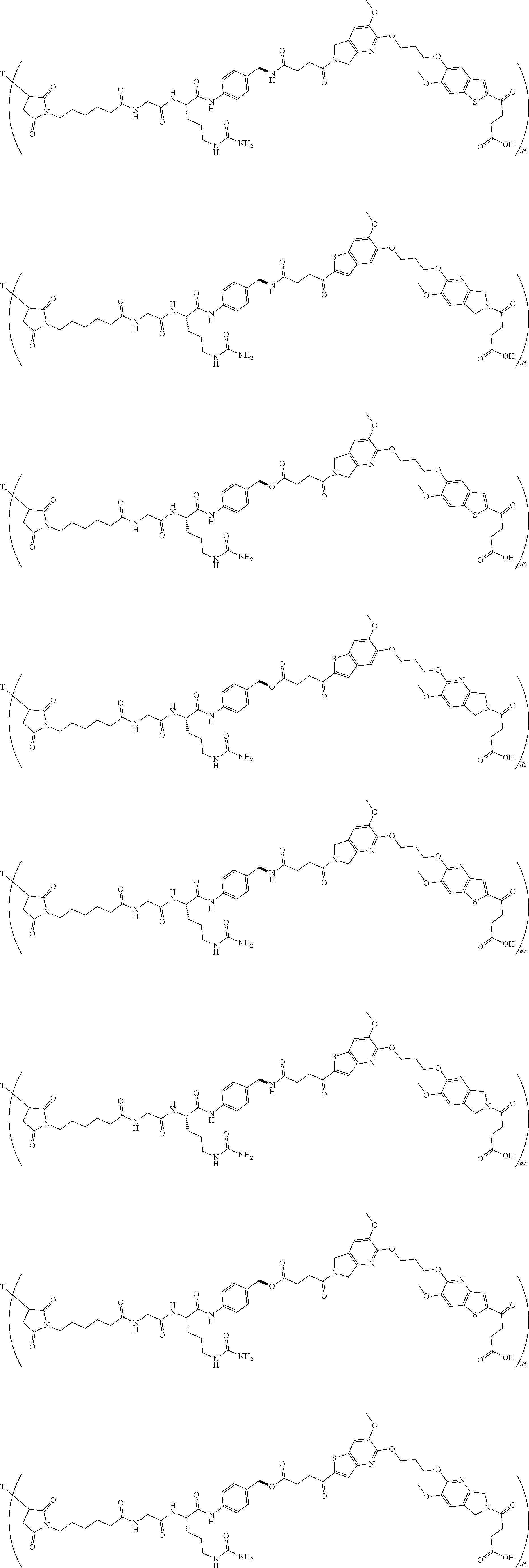 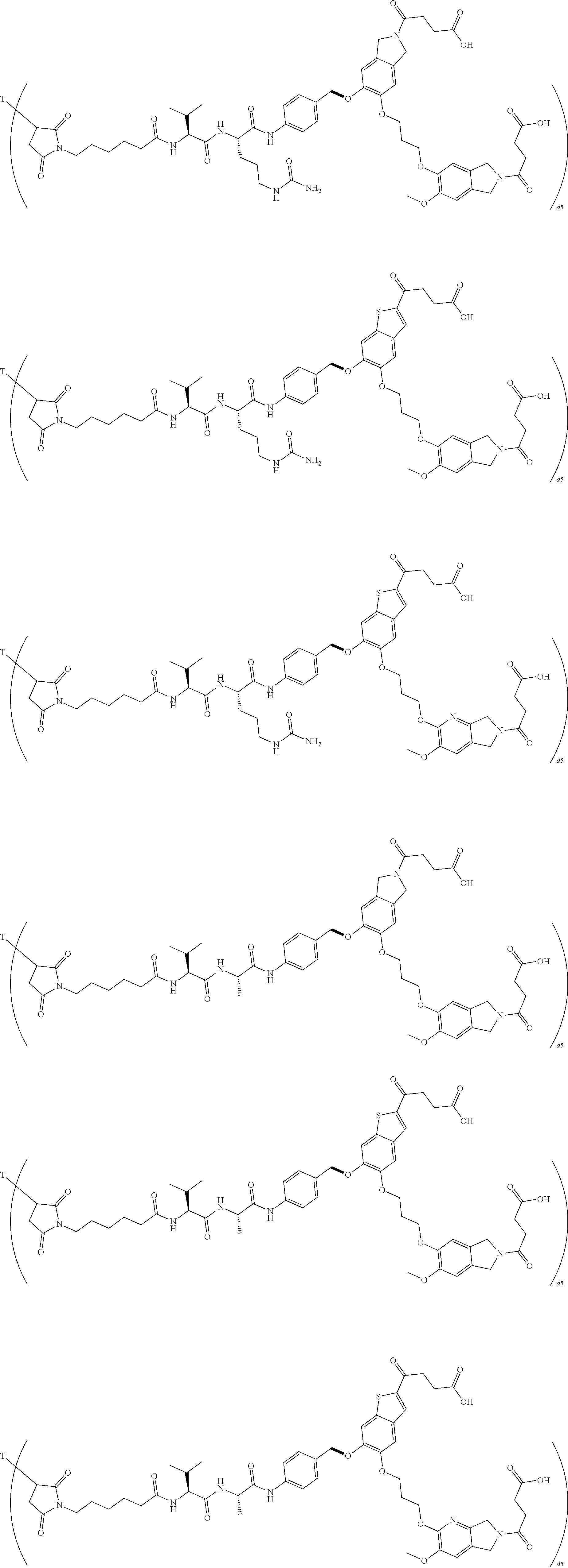 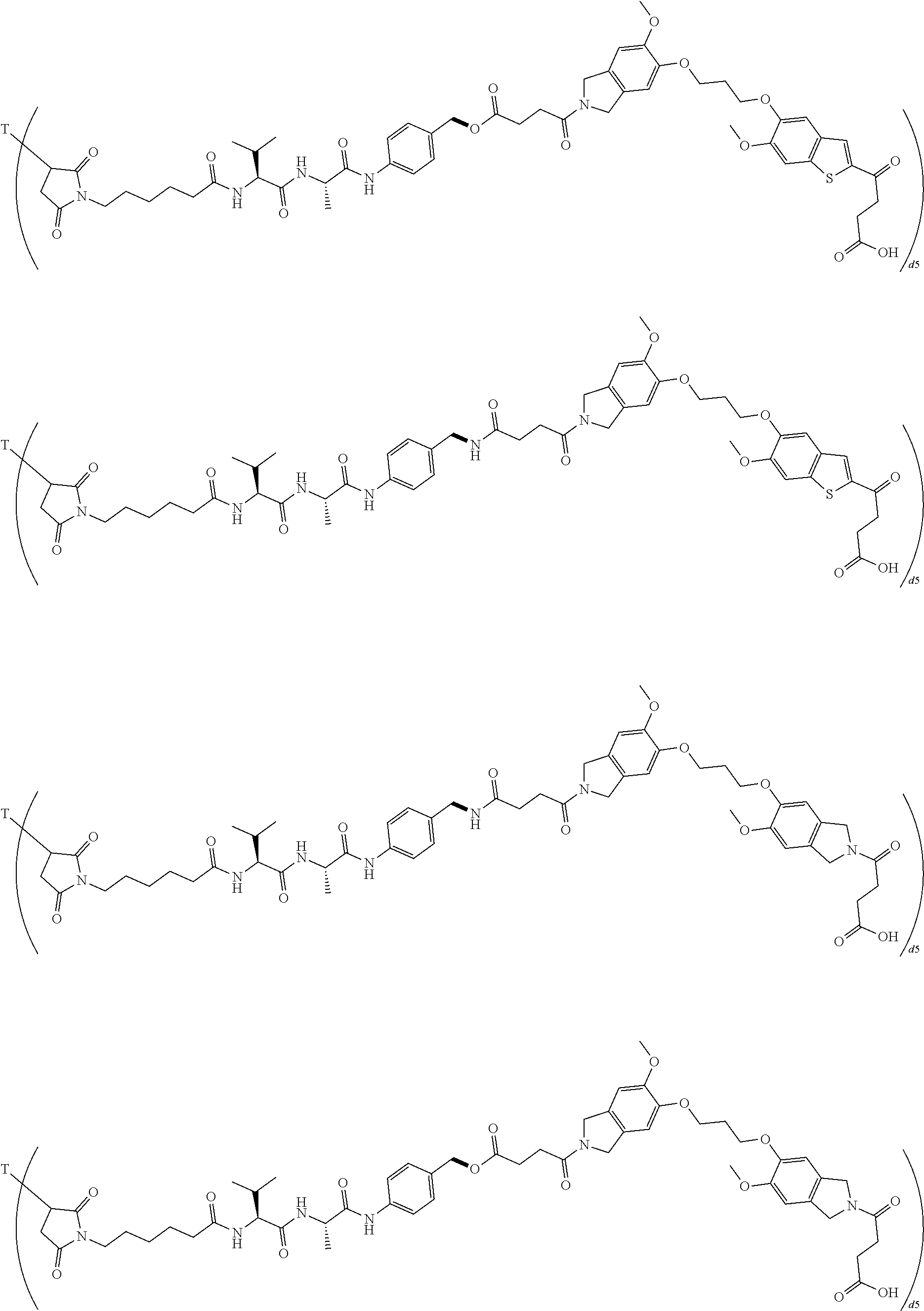 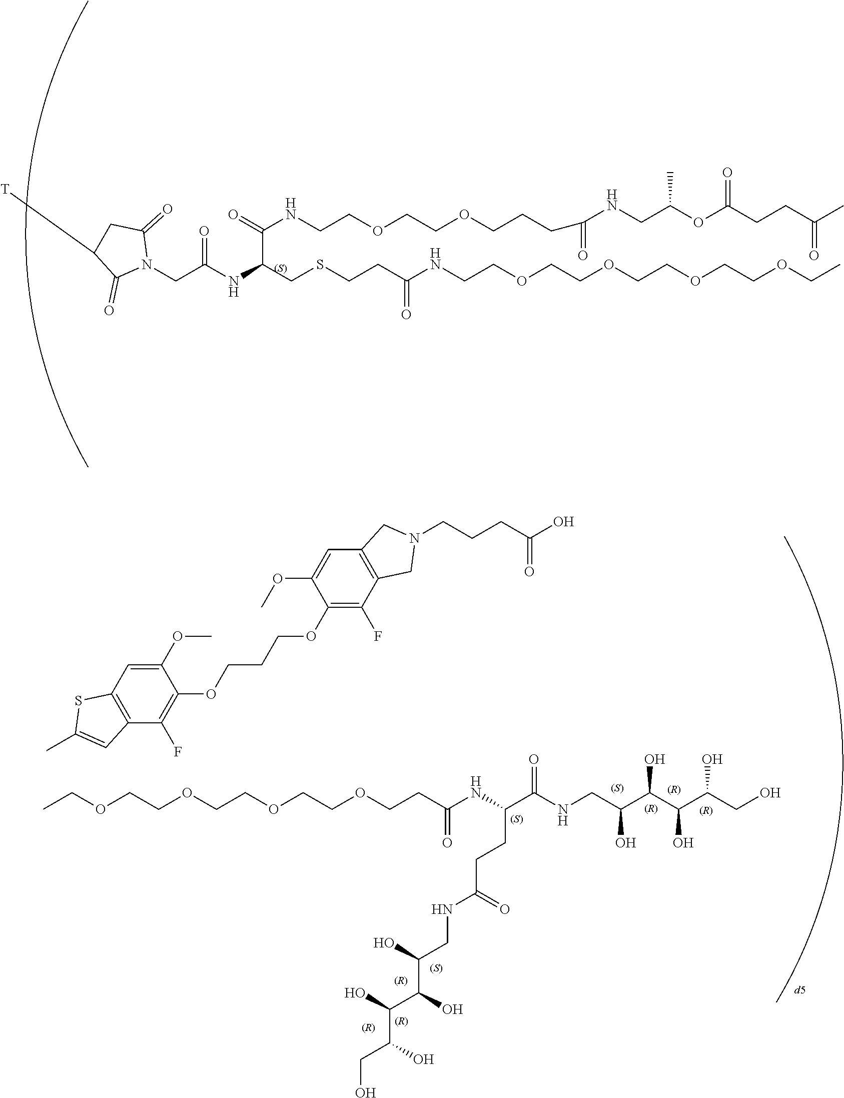 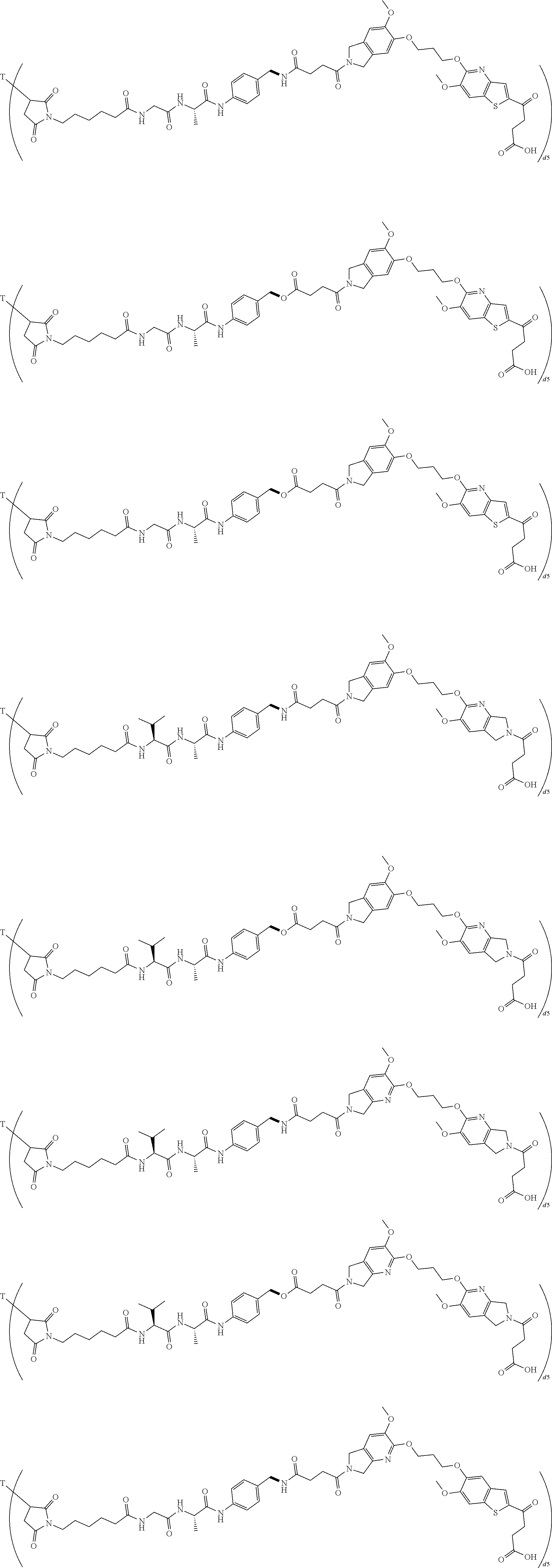 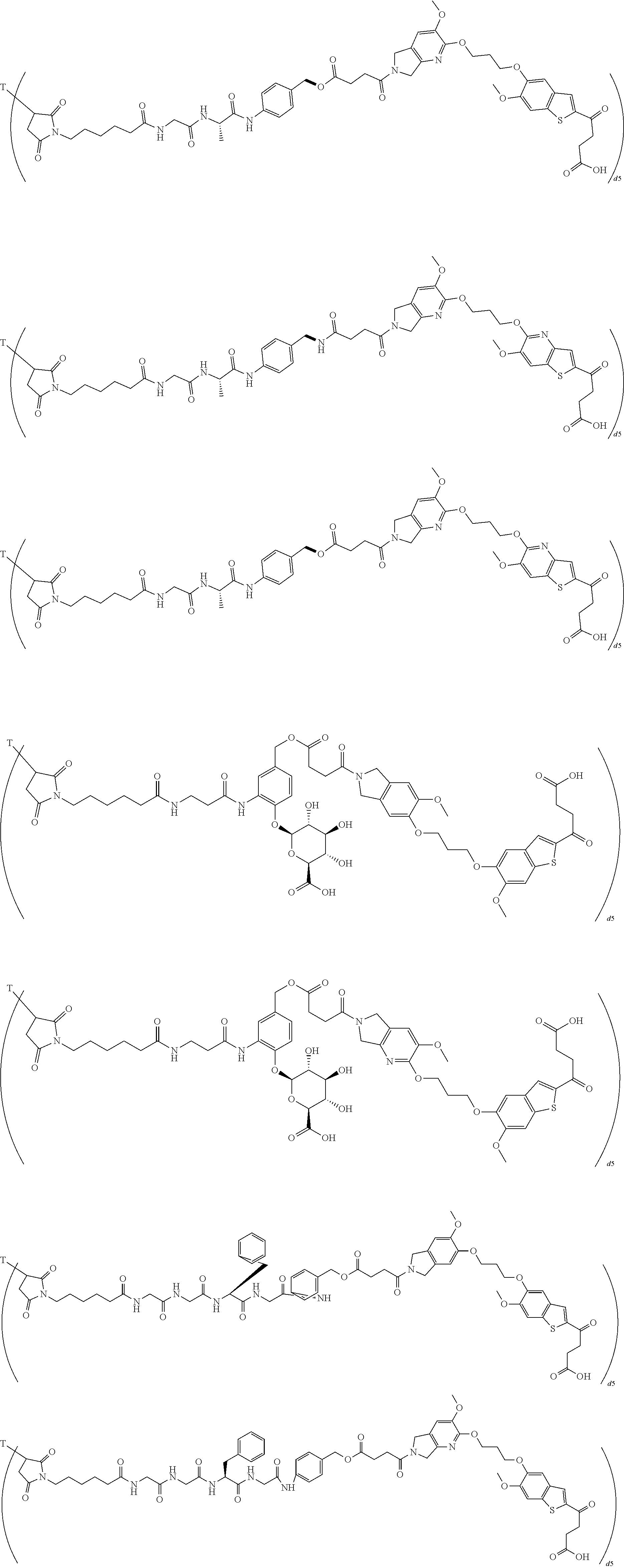 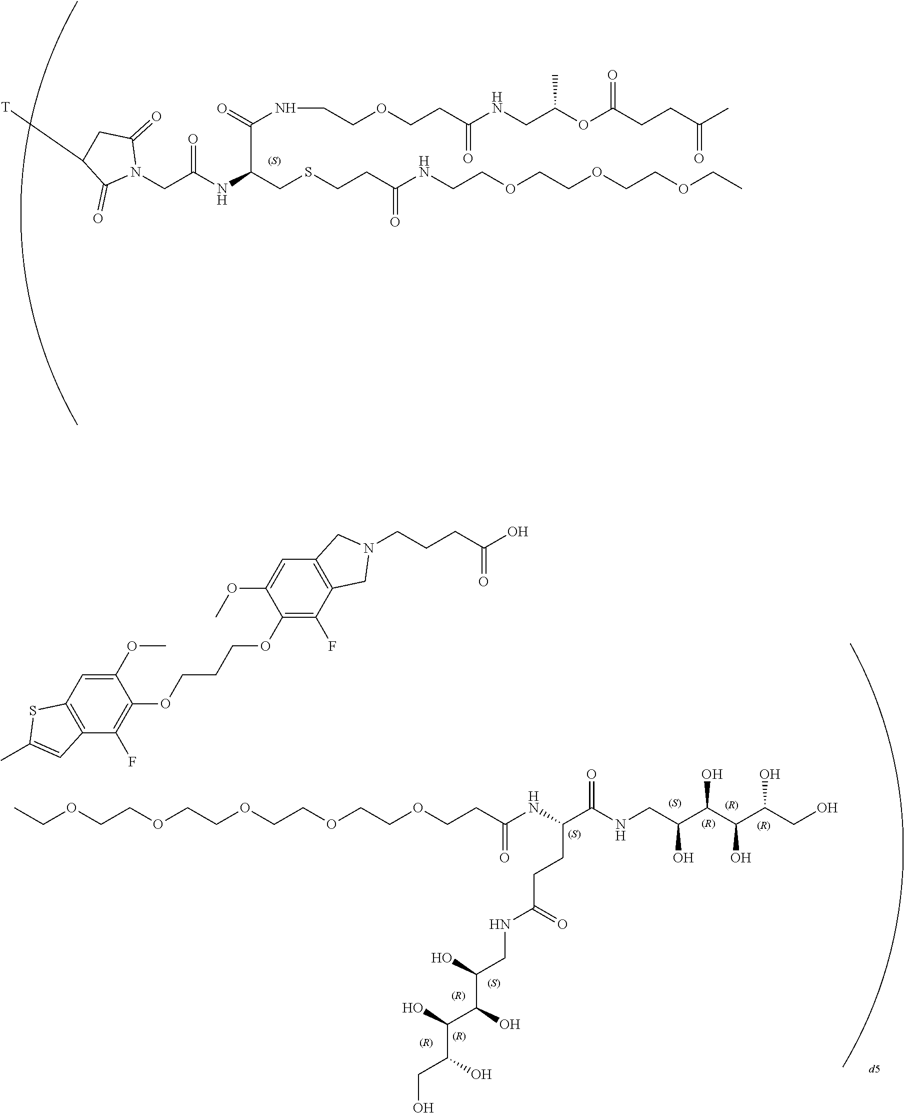 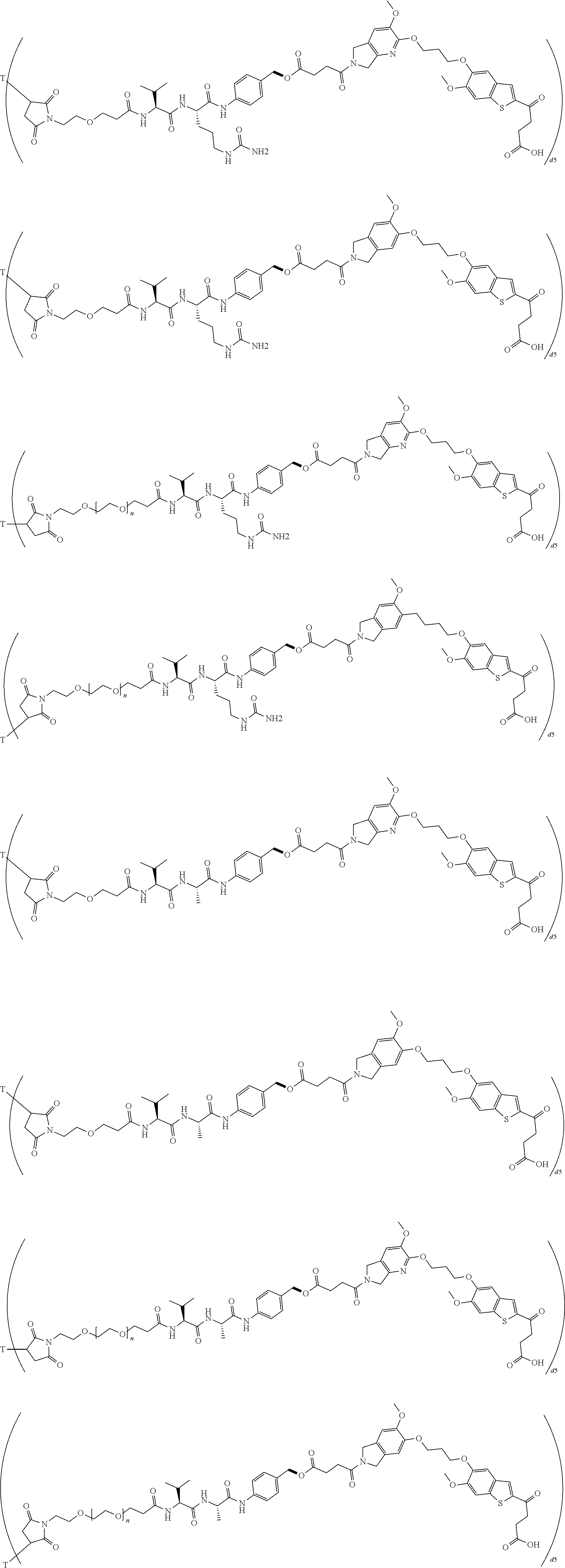 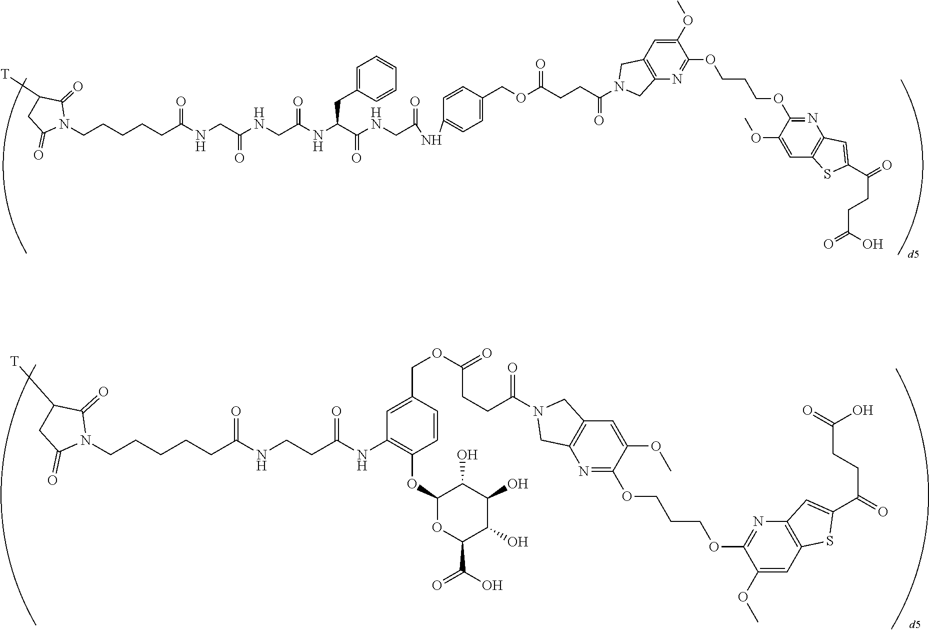 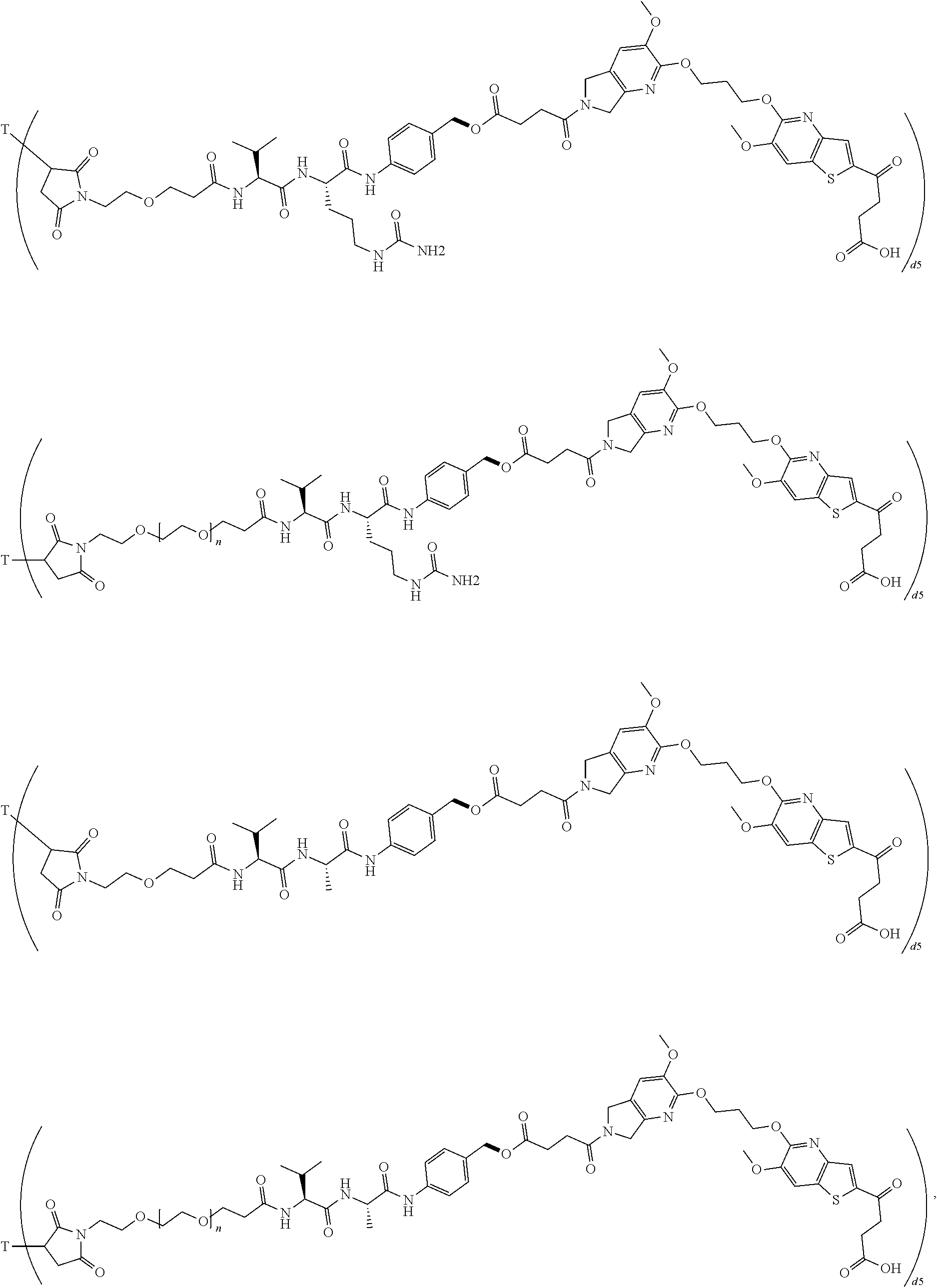 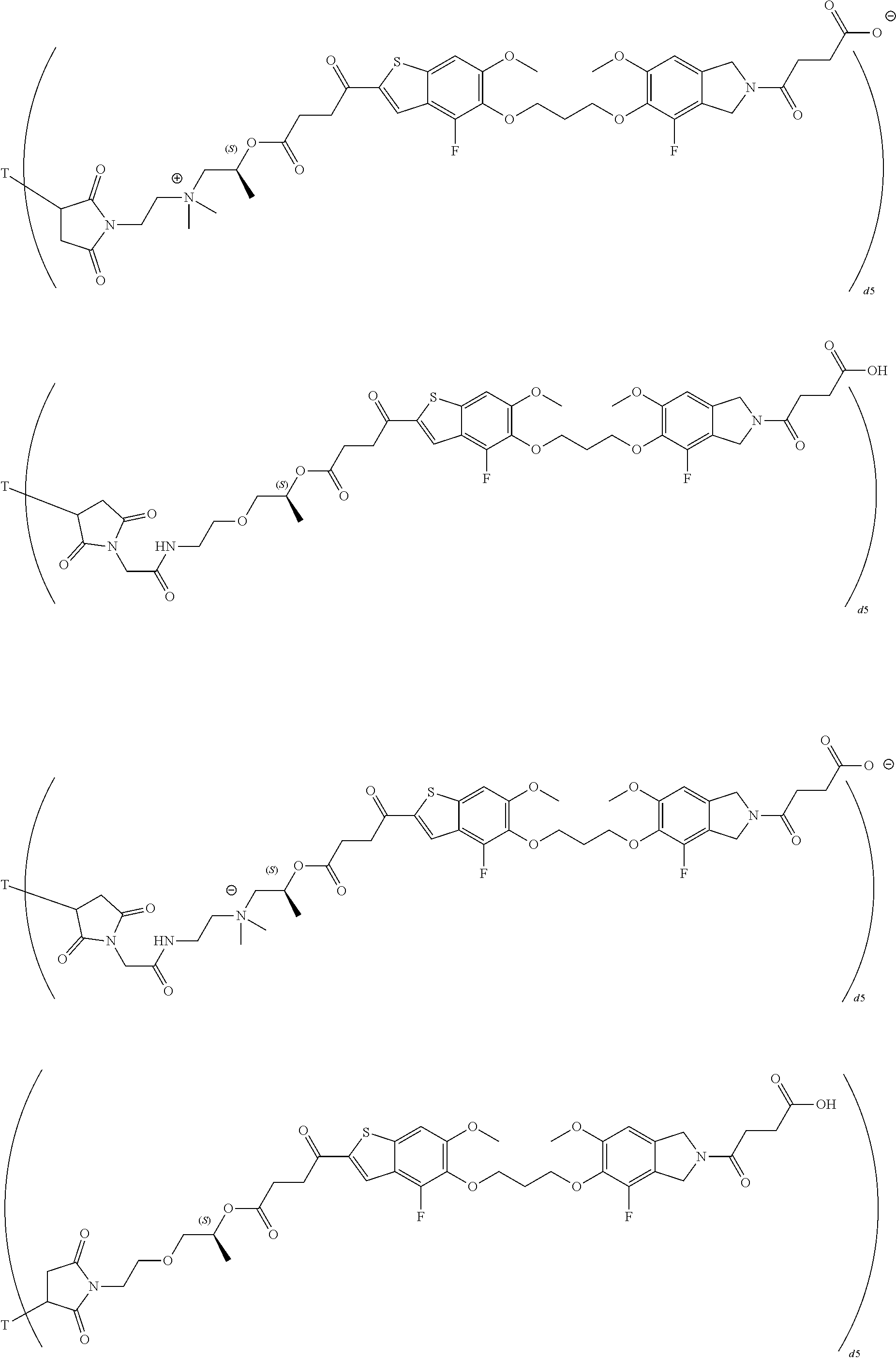 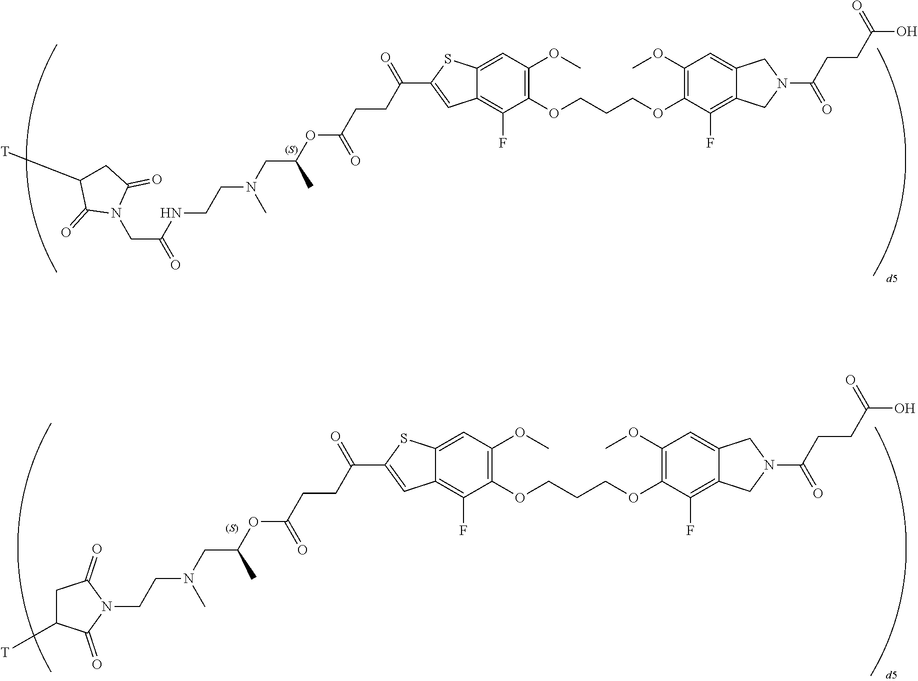 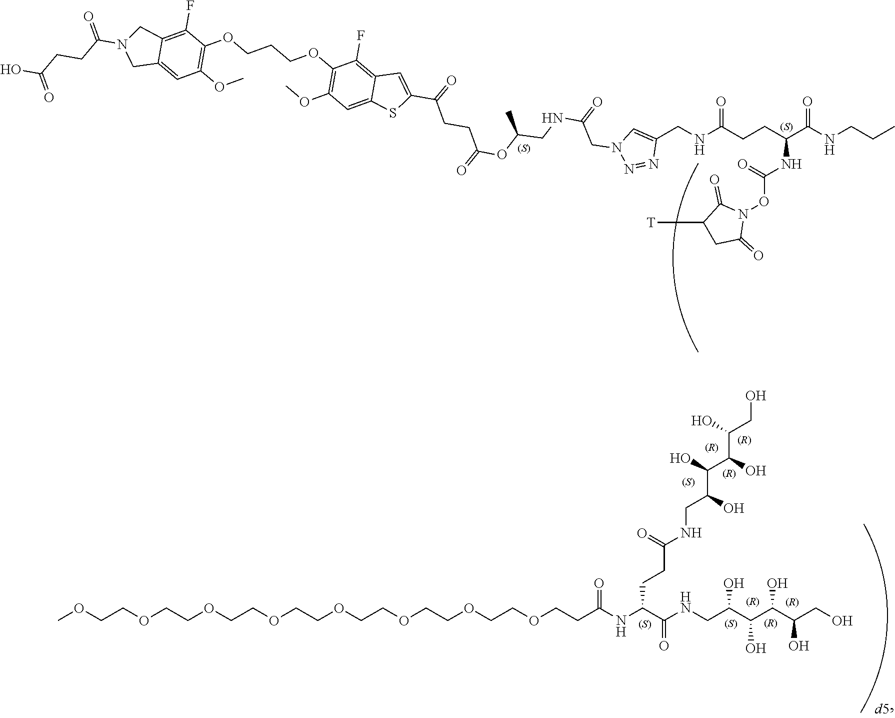
wherein d5 is an integer ranging from 1 to 20, such as from 6 to 10, such as 8.

54. The compound-linker construct or conjugate of any one of the preceding claims, wherein the targeting moiety T is selected from the group consisting of an antibody, an antibody fragment, a nucleic acid based molecule, a carbohydrate, a peptide, or a modified peptide;
preferably, the targeting moiety T is an antibody or an antigen-binding fragment, which is designed to target the Human Epidermal Growth Factor Receptor (EGFR), a plasminogen activator, a cytotoxic T-lymphocyte associated antigen (CTLA) such as CTLA-4, PD-1, PD-LI, KIR, TIM3, VISTA, TIGIT, LAG3, OX40, RORI, ROR2, vascular endothelial growth factor (VEGF), fibroblast growth factor receptor (FGFR), platelet-derived growth factor (PDGF), transforming growth factor (TGF), neurotrophic factors, a nerve growth factor, platelet-derived growth factor (PDGF), interleukin receptors, transforming growth factor (TGF), estrogen receptor, progesterone receptor, c-Kit, cMET, ErbB2/Her2, ErbB3/Her3, ErbB4/Her4, CD3, CD20, CD22, CD30, CD33, CD40, CD47, CD73, CD79, CD123, CD133, CD166, CD137, Claudin18.2, the mesothelin protein, EpCAM, FLT3, PSMA, PSCA, STEAP, CEA, folate receptor, the CD39/CD73 receptors, adenosine receptors, SLC34A2 gene product, the EphA2 tyrosine kinase, the Mucl/Muc16 cell-surface antigens, ALK, AFP, bcr-Abl, PAP;
more preferably, the targeting moiety T is selected from the group consisting of Trastuzumab, Disitamab, Cetuximab, Rituximab, Bevacizumab, Epratuzumab, Veltuzumab, Labetuzumab, Atezolizumab;
more preferably, the targeting moiety T is an antibody against an antigen, such as, for example, B7-H4, B7-H3, CD11b, CD103, CA125, CDH6, CD33, CD73, Claudin18.2, CXCR2, CEACAM5, Clec9A, CSFR1, DEC205, EGFR, FAP, fibronectin-EDB, FGFRI, FGFR2, FGFR3, FGFR4, GCC (GUCY2C), HER2, LIVI, LY6E, NaPi2b, c-Met, mesothelin, NOTCHI, NOTCH2, NOTCH3, NOTCH4, PD-L1, PTK7, c-Kit, MUC1, MUC13. and 5T4.

55. The compound-linker construct or conjugate of any one of the preceding claims, wherein the conjugate is: 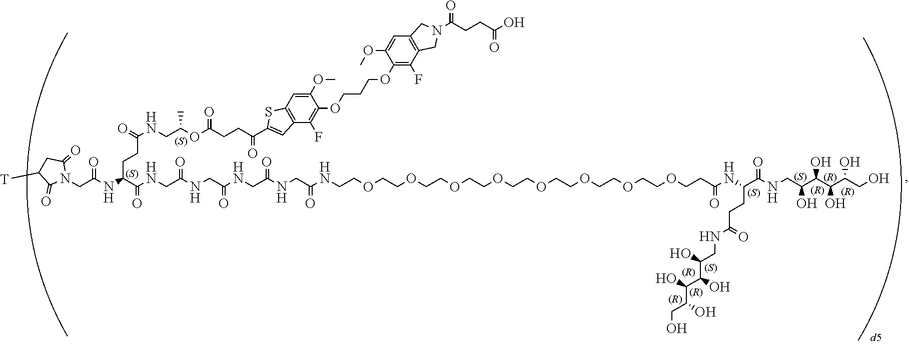
wherein T is an anti-HER2 antibody, such as Trastuzumab or Disitamab, and d5 is an integer of 6-8, such as 7 or 8; 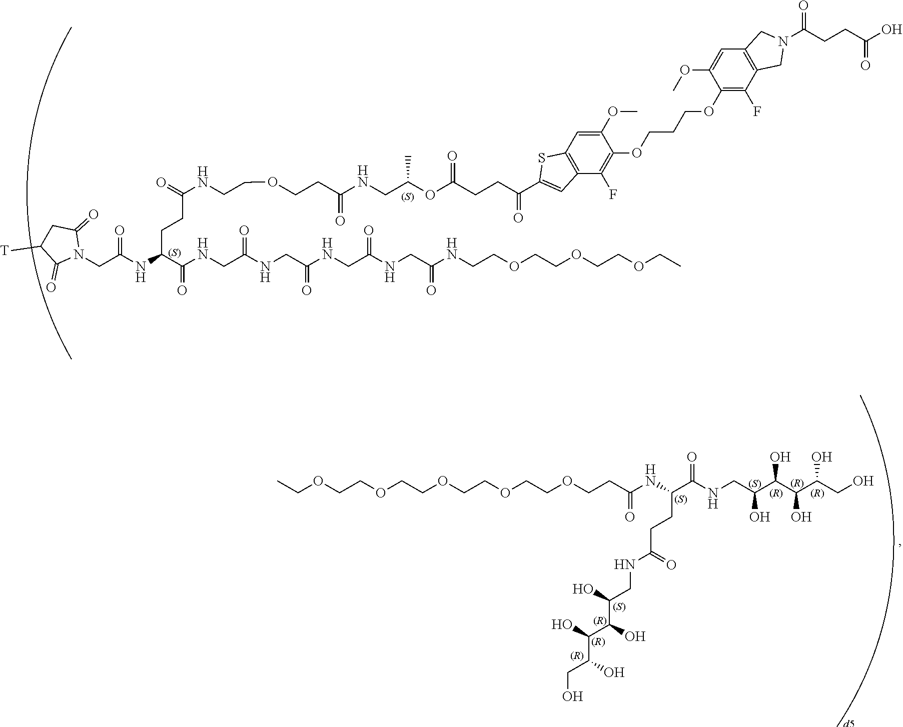
wherein T is an anti-HER2 antibody, such as Trastuzumab or Disitamab, and d5 is an integer of 6-8, such as 7 or 8; 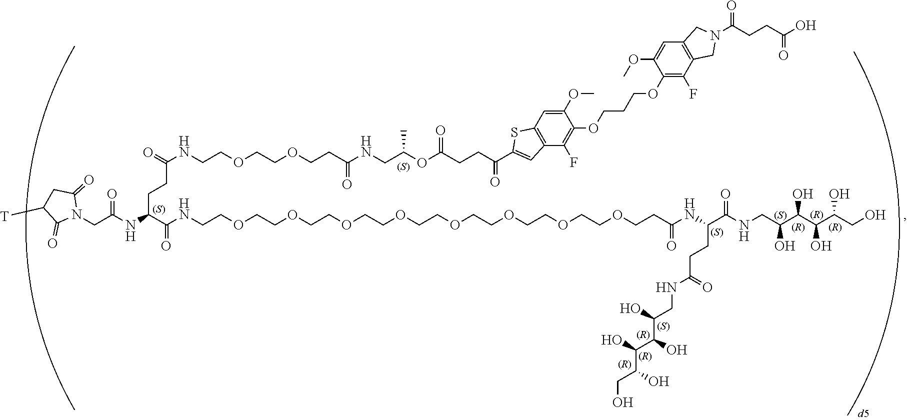
wherein T is an anti-HER2 antibody, such as Trastuzumab or Disitamab, and d5 is an integer of 6-8, such as 7 or 8; 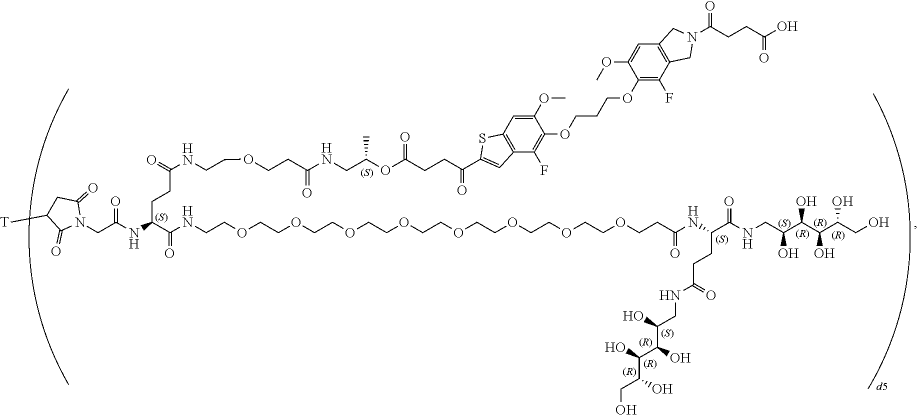
wherein T is an anti-HER2 antibody, such as Trastuzumab or Disitamab, and d5 is an integer of 6-8, such as 7 or 8; 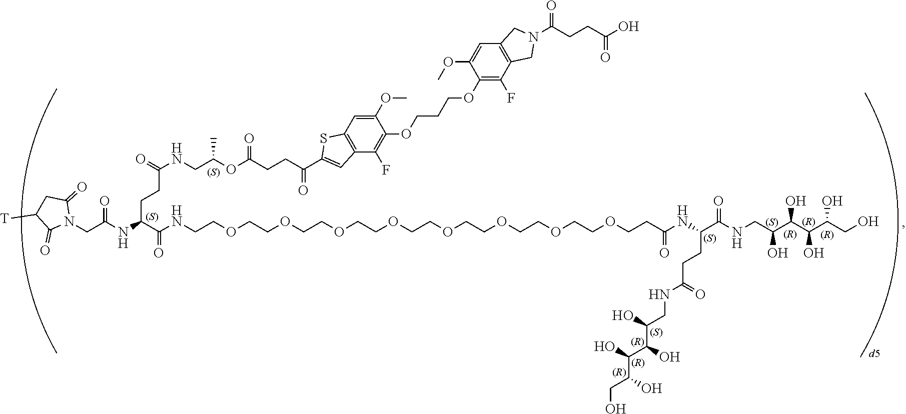
wherein T is an anti-HER2 antibody, such as Trastuzumab or Disitamab, and d5 is an integer of 6-8, such as 7 or 8; 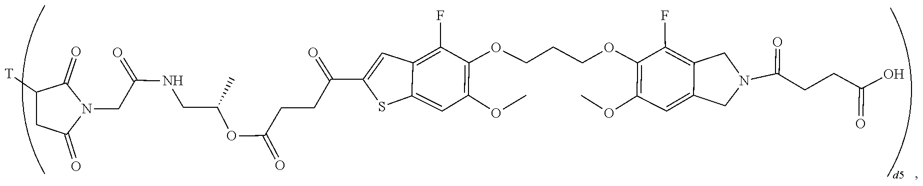
wherein T is an anti-HER2 antibody, such as Trastuzumab or Disitamab, and d5 is an integer of 6-8, such as 7 or 8; 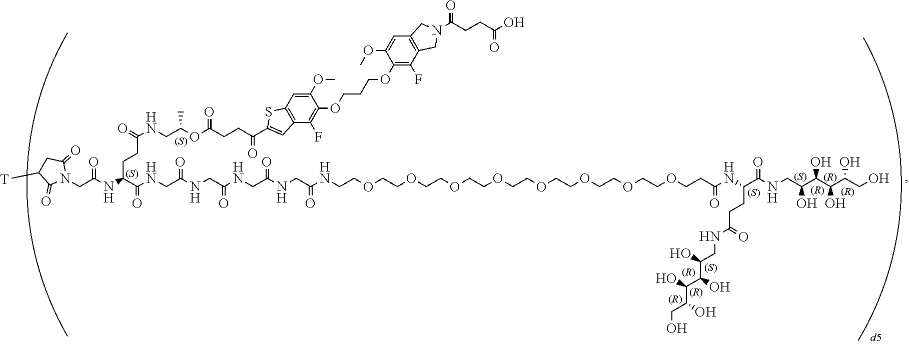
wherein T is anti-CD73 antibody having a heavy chain of the amino acid sequence as shown in SEQ ID NO: 1 or 3 and a light chain of the amino acid sequence as shown in SEQ ID NO: 2, and d5 is an integer of 6-8, such as 7 or 8; 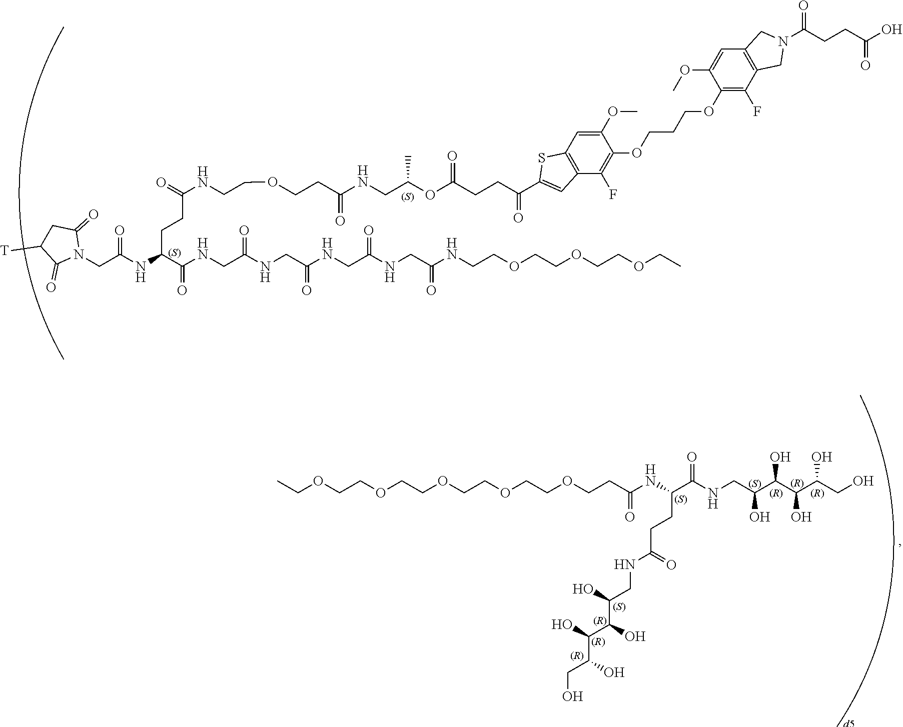
wherein T is anti-CD73 antibody having a heavy chain of the amino acid sequence as shown in SEQ ID NO: 1 or 3 and a light chain of the amino acid sequence as shown in SEQ ID NO: 2, and d5 is an integer of 6-8, such as 7 or 8; 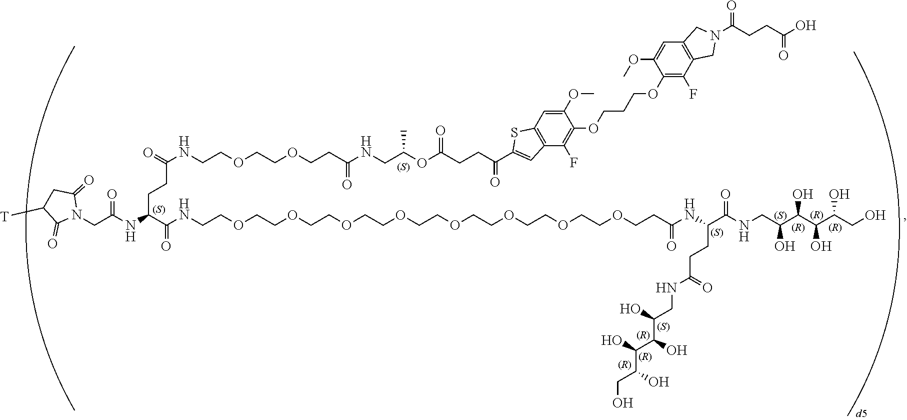
wherein T is anti-CD73 antibody having a heavy chain of the amino acid sequence as shown in SEQ ID NO: 1 or 3 and a light chain of the amino acid sequence as shown in SEQ ID NO: 2, and d5 is an integer of 6-8, such as 7 or 8; 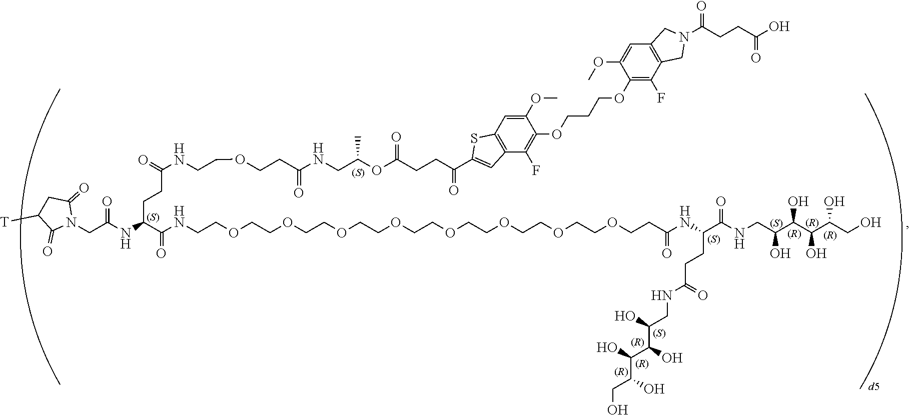
wherein T is anti-CD73 antibody having a heavy chain of the amino acid sequence as shown in SEQ ID NO: 1 or 3 and a light chain of the amino acid sequence as shown in SEQ ID NO: 2, and d5 is an integer of 6-8, such as 7 or 8; 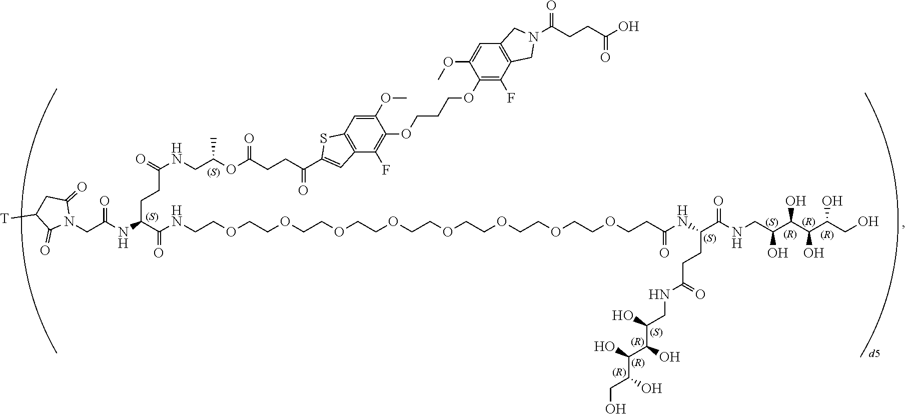
wherein T is anti-CD73 antibody having a heavy chain of the amino acid sequence as shown in SEQ ID NO: 1 or 3 and a light chain of the amino acid sequence as shown in SEQ ID NO: 2, and d5 is an integer of 6-8, such as 7 or 8; 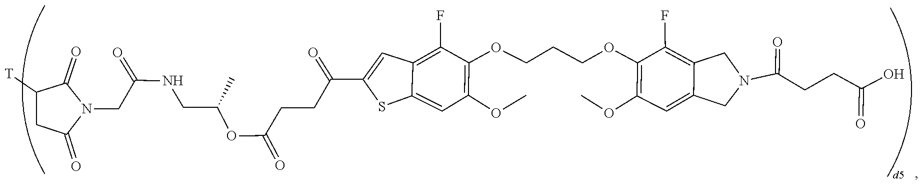
wherein T is anti-CD73 antibody having a heavy chain of the amino acid sequence as shown in SEQ ID NO: 1 or 3 and a light chain of the amino acid sequence as shown in SEQ ID NO: 2, and d5 is an integer of 6-8, such as 7 or 8; 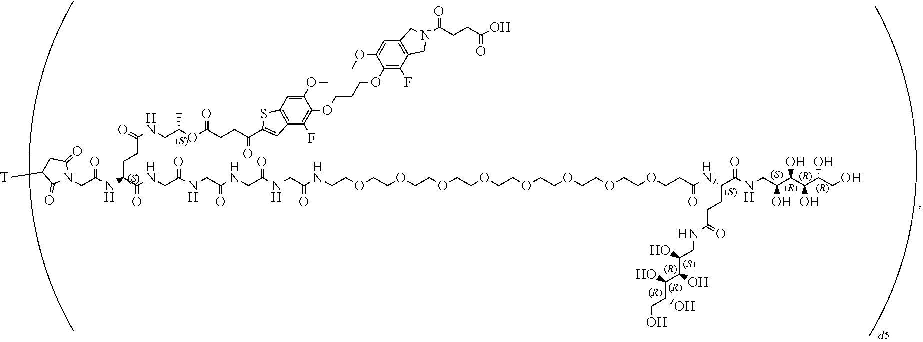
wherein T is an anti-PD-L1 antibody, such as Atezolizumab, and d5 is an integer of 6-8, such as 7 or 8; 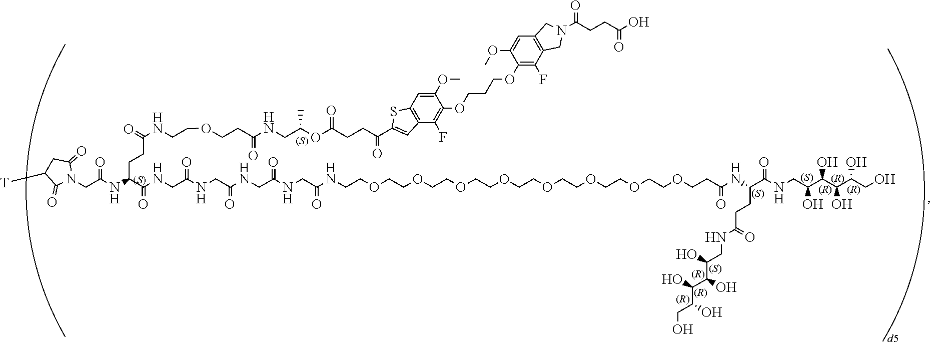
wherein T is an anti-PD-L1 antibody, such as Atezolizumab, and d5 is an integer of 6-8, such as 7 or 8; 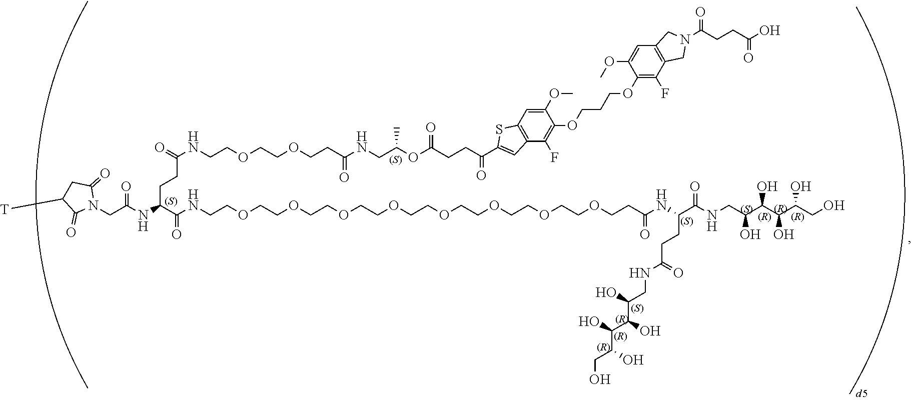
wherein T is an anti-PD-L1 antibody, such as Atezolizumab, and d5 is an integer of 6-8, such as 7 or 8; 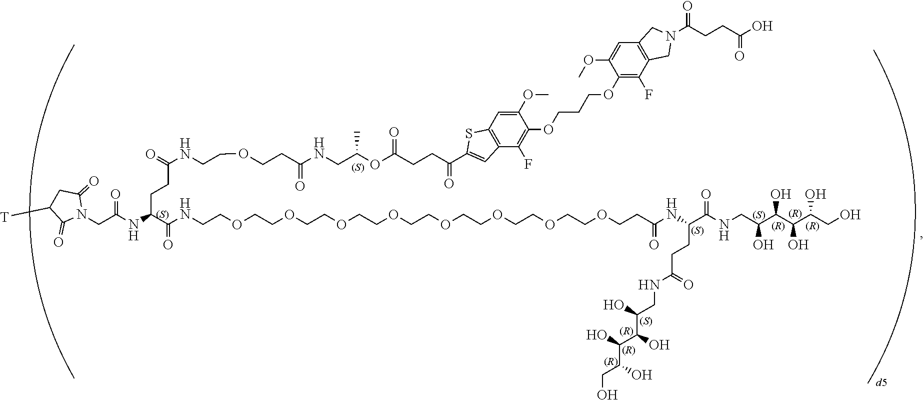
wherein T is an anti-PD-Li antibody, such as Atezolizumab, and d5 is an integer of 6-8, such as 7 or 8; 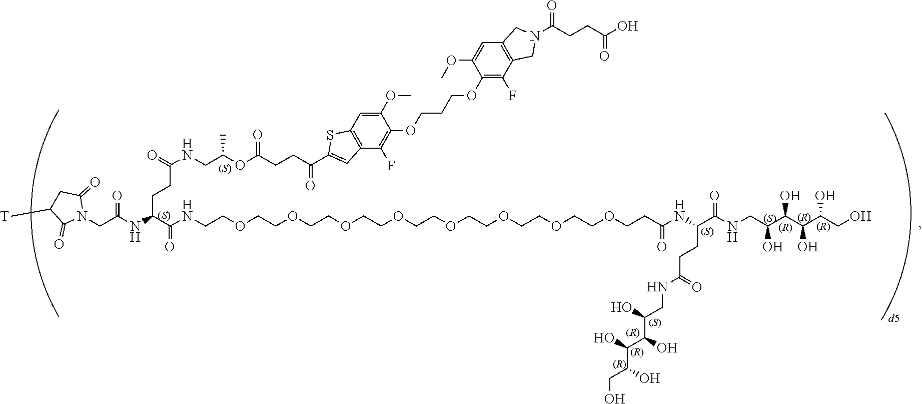
wherein T is an anti-PD-L1 antibody, such as Atezolizumab, and d5 is an integer of 6-8, such as 7 or 8; 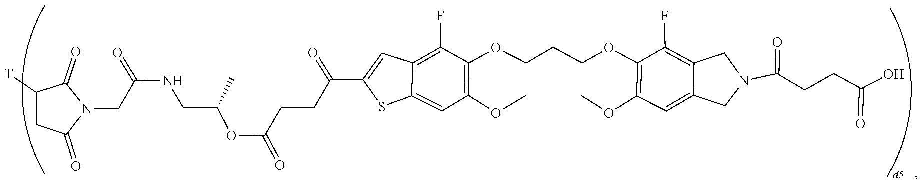
wherein T is an anti-PD-L1 antibody, such as Atezolizumab, and d5 is an integer of 6-8, such as 7 or 8; 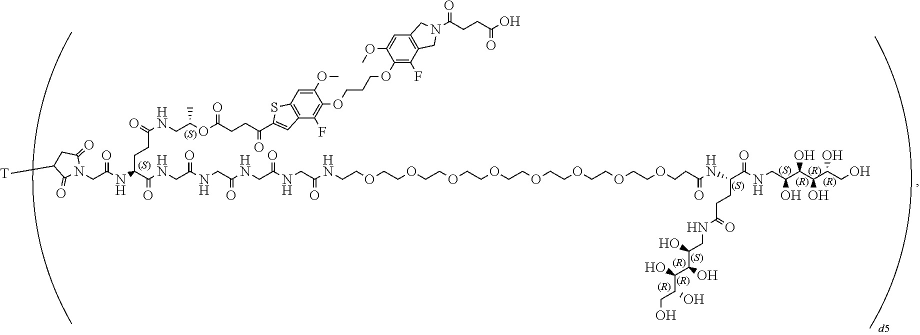
wherein T is an anti-cMet antibody, for example having a heavy chain of the amino acid sequence as shown in SEQ ID NO: 4 and a light chain of the amino acid sequence as shown in SEQ ID NO: 5; or for example having a heavy chain of the amino acid sequence as shown in SEQ ID NO: 8 and a light chain of the amino acid sequence as shown in SEQ ID NO: 9; and d5 is an integer of 6-8, such as 7 or 8; 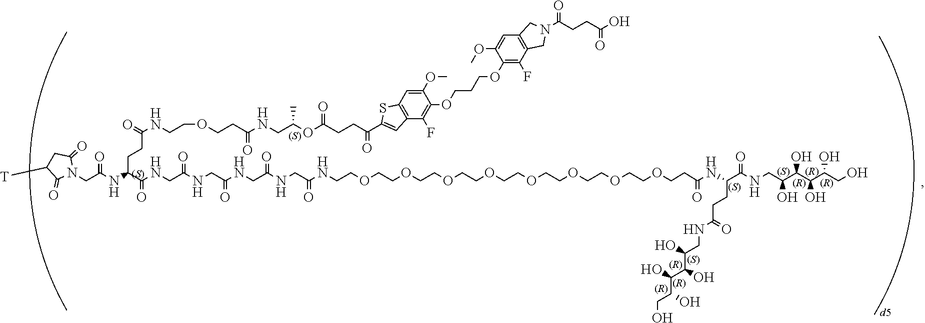
wherein T is an anti-cMet antibody, for example having a heavy chain of the amino acid sequence as shown in SEQ ID NO: 4 and a light chain of the amino acid sequence as shown in SEQ ID NO: 5; or for example having a heavy chain of the amino acid sequence as shown in SEQ ID NO: 8 and a light chain of the amino acid sequence as shown in SEQ ID NO: 9; and d5 is an integer of 6-8, such as 7 or 8; 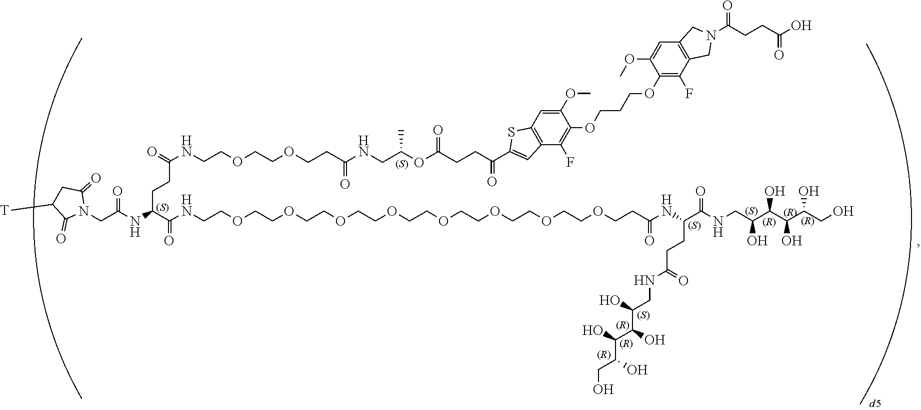
wherein T is an anti-cMet antibody, for example having a heavy chain of the amino acid sequence as shown in SEQ ID NO: 4 and a light chain of the amino acid sequence as shown in SEQ ID NO: 5; or for example having a heavy chain of the amino acid sequence as shown in SEQ ID NO: 8 and a light chain of the amino acid sequence as shown in SEQ ID NO: 9; and d5 is an integer of 6-8, such as 7 or 8; 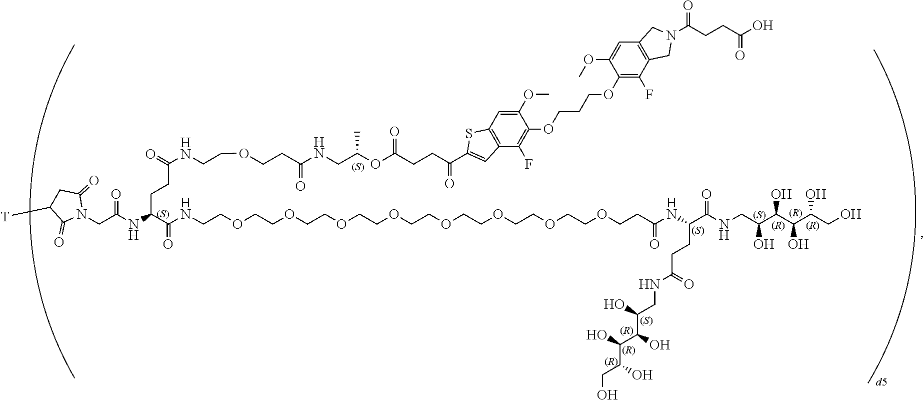
wherein T is an anti-cMet antibody, for example having a heavy chain of the amino acid sequence as shown in SEQ ID NO: 4 and a light chain of the amino acid sequence as shown in SEQ ID NO: 5; or for example having a heavy chain of the amino acid sequence as shown in SEQ ID NO: 8 and a light chain of the amino acid sequence as shown in SEQ ID NO: 9; and d5 is an integer of 6-8, such as 7 or 8; 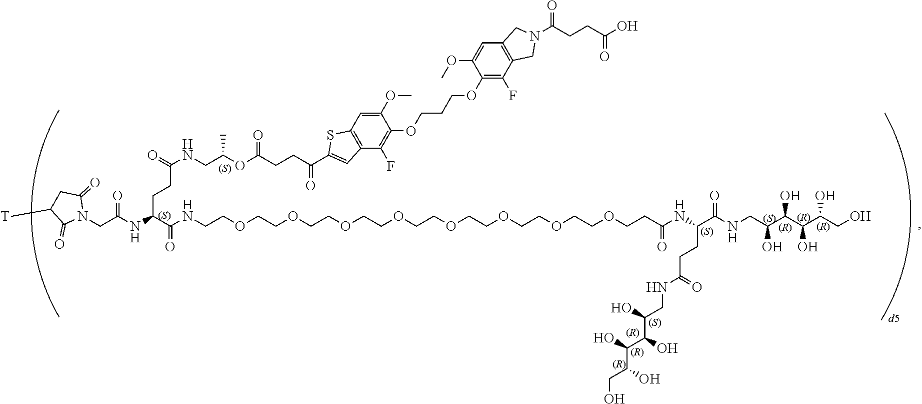
wherein T is an anti-cMet antibody, for example having a heavy chain of the amino acid sequence as shown in SEQ ID NO: 4 and a light chain of the amino acid sequence as shown in SEQ ID NO: 5; or for example having a heavy chain of the amino acid sequence as shown in SEQ ID NO: 8 and a light chain of the amino acid sequence as shown in SEQ ID NO: 9; and d5 is an integer of 6-8, such as 7 or 8; 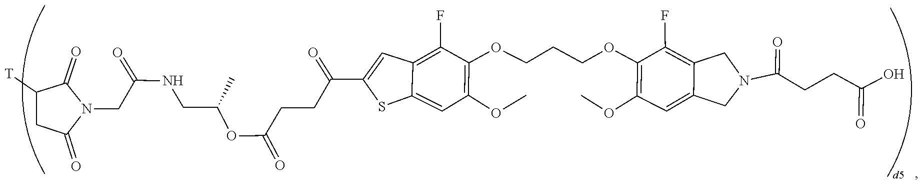
wherein T is an anti-cMet antibody, for example having a heavy chain of the amino acid sequence as shown in SEQ ID NO: 4 and a light chain of the amino acid sequence as shown in SEQ ID NO: 5; or for example having a heavy chain of the amino acid sequence as shown in SEQ ID NO: 8 and a light chain of the amino acid sequence as shown in SEQ ID NO: 9; and d5 is an integer of 6-8, such as 7 or 8; 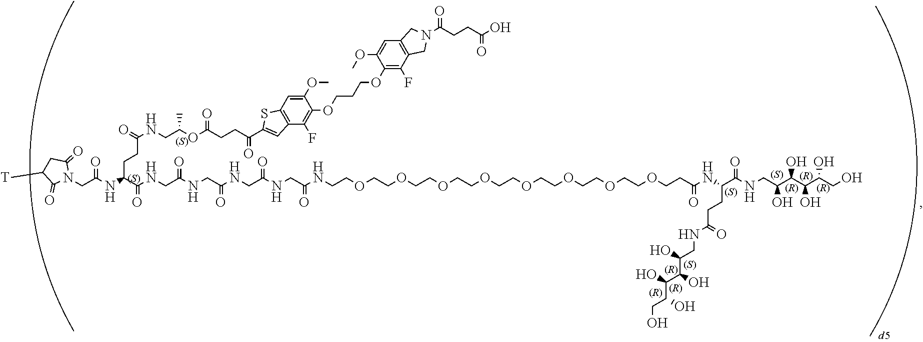
wherein T is an anti-mesothelin antibody, for example having a heavy chain of the amino acid sequence as shown in SEQ ID NO: 6 and a light chain of the amino acid sequence as shown in SEQ ID NO: 7, and d5 is an integer of 6-8, such as 7 or 8; 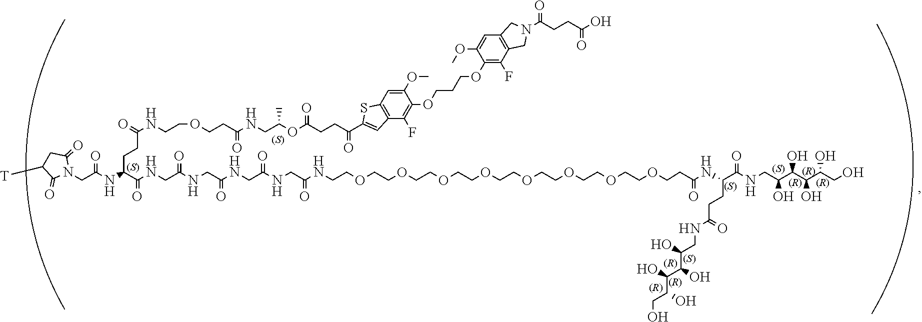
wherein T is an anti-mesothelin antibody, for example having a heavy chain of the amino acid sequence as shown in SEQ ID NO: 6 and a light chain of the amino acid sequence as shown in SEQ ID NO: 7, and d5 is an integer of 6-8, such as 7 or 8; or 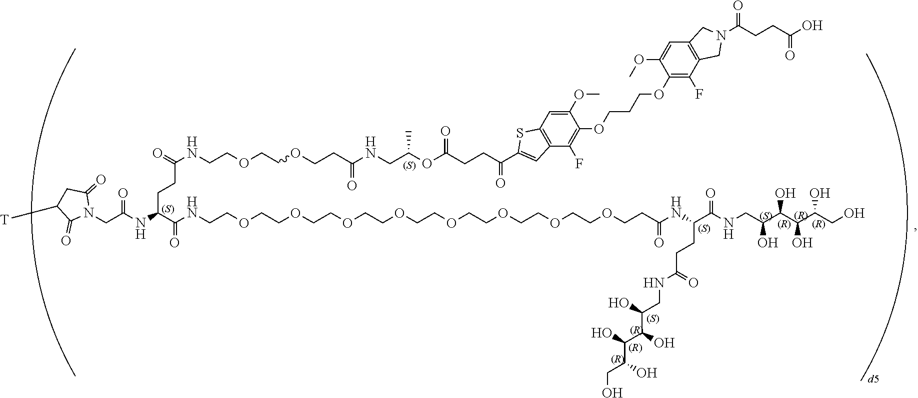
wherein T is an anti-mesothelin antibody, for example having a heavy chain of the amino acid sequence as shown in SEQ ID NO: 6 and a light chain of the amino acid sequence as shown in SEQ ID NO: 7, and d5 is an integer of 6-8, such as 7 or 8; 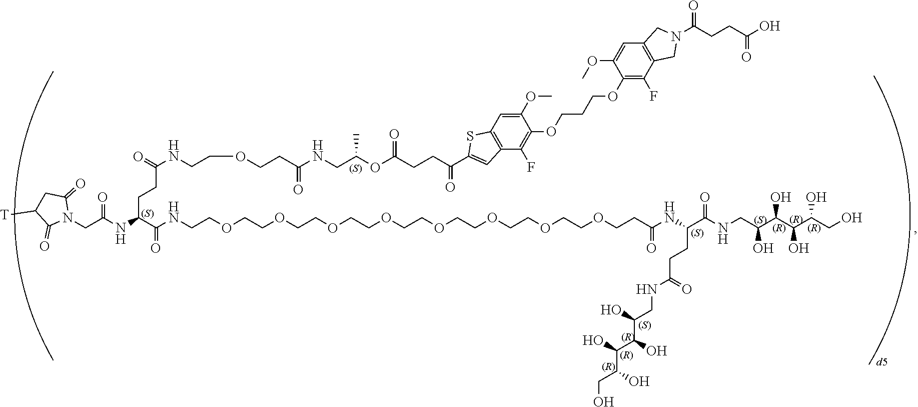
wherein T is an anti-mesothelin antibody, for example having a heavy chain of the amino acid sequence as shown in SEQ ID NO: 6 and a light chain of the amino acid sequence as shown in SEQ ID NO: 7, and d5 is an integer of 6-8, such as 7 or 8; 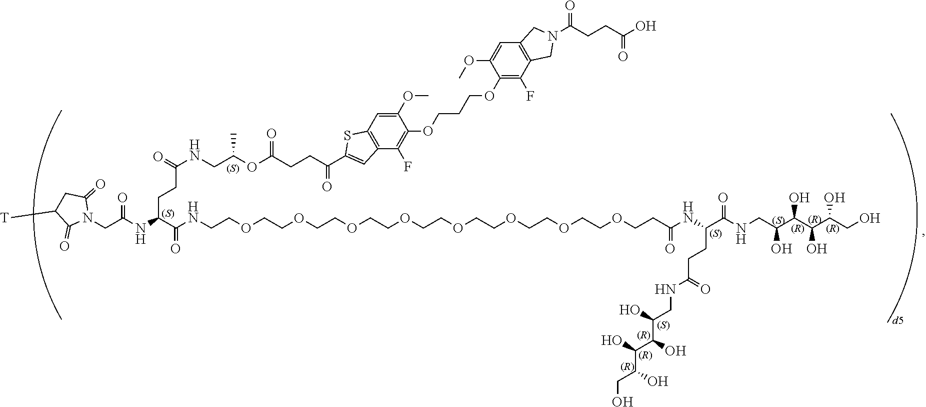
wherein T is an anti-mesothelin antibody, for example having a heavy chain of the amino acid sequence as shown in SEQ ID NO: 6 and a light chain of the amino acid sequence as shown in SEQ ID NO: 7, and d5 is an integer of 6-8, such as 7 or 8; 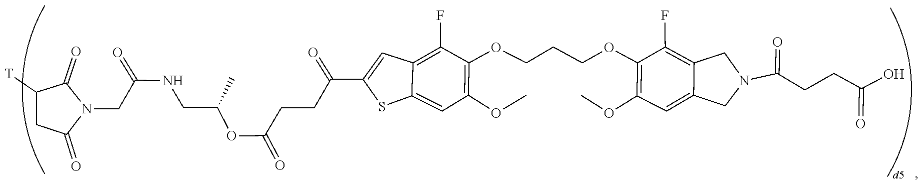
wherein T is an anti-mesothelin antibody, for example having a heavy chain of the amino acid sequence as shown in SEQ ID NO: 6 and a light chain of the amino acid sequence as shown in SEQ ID NO: 7, and d5 is an integer of 6-8, such as 7 or 8.

56. A pharmaceutical composition comprising a therapeutically effective amount of the compound-linker construct or the conjugate of any one of the preceding claims and at least one pharmaceutically acceptable excipient.

57. The pharmaceutical composition of claim 56 further comprising at least one immuno-modulator or at least one immunostimulatory agent.

58. The pharmaceutical composition of claim 56 or 57, further comprising at least one additional active agents selected from STING agonist compounds, anti-viral compounds, antigens, adjuvants, CTLA-4 and PD-1 pathway antagonists and other immunomodulatory agents, lipids, liposomes, peptides, anti-cancer agents, and chemotherapeutic agents.

59. Use of the compound-linker construct or the conjugate of any one of the preceding claims, and/or the pharmaceutical composition of any one of claims 56-58 for the manufacture of a medicament.

60. The use of claim 59, wherein the medicament is used for inducing an immune response in a subject.

61. The use of claim 59, wherein the medicament is used for inducing STING-dependent type I interferon production in a subject.

62. The use of claim 59, wherein the medicament is used for inducing a STING-dependent cytokine production in a subject.

63. The use of claim 59, wherein the medicament is used for treating a cell proliferation disorder in a subject.

64. The use of claim 63, wherein the cell proliferation disorder is cancer.

65. The compound-linker construct or the conjugate of any one of the preceding claims, and/or the pharmaceutical composition of any one of claims 56-58, for use in therapy.

66. The compound-linker construct or the conjugate of any one of the preceding claims, and/or the pharmaceutical composition of any one of claims 56-58, for use in inducing an immune response in a subject.

67. The compound-linker construct or the conjugate of any one of the preceding claims, and/or the pharmaceutical composition of any one of claims 56-58, for use in inducing STING-dependent type I interferon production in a subject.

68. The compound-linker construct or the conjugate of any one of the preceding claims, and/or the pharmaceutical composition of any one of claims 56-58, for use in inducing a STING-dependent cytokine production in a subject.

69. The compound-linker construct or the conjugate of any one of the preceding claims, and/or the pharmaceutical composition of any one of claims 56-58, for use in treating a cell proliferation disorder in a subject.

70. The use of claim 69, wherein the cell proliferation disorder is cancer.

71. The compound-linker construct or the conjugate of any one of the preceding claims, and/or the pharmaceutical composition of any one of claims 56-58, for use as a STING agonist.

72. The compound-linker construct or the conjugate of any one of the preceding claims, and/or the pharmaceutical composition of any one of claims 56-58, for use as a medicament.

73. A method of inducing an immune response in a subject, said method comprising administering a therapeutically effective amount of the compound-linker construct or the conjugate of any one of the preceding claims, and/or the pharmaceutical composition of any one of claims 56-58 to the subject.

74. A method of inducing STING-dependent type I interferon production in a subject, said method comprising administering a therapeutically effective amount of the compound-linker construct or the conjugate of any one of the preceding claims, and/or the pharmaceutical composition of any one of claims 56-58 to the subject.

75. A method of inducing a STING-dependent cytokine production in a subject, said method comprising administering a therapeutically effective amount of the compound-linker construct or the conjugate of any one of the preceding claims, and/or the pharmaceutical composition of any one of claims 56-58 to the subject.

76. A method of treating a cell proliferation disorder in a subject, said method comprising administering a therapeutically effective amount of the compound-linker construct or the conjugate of any one of the preceding claims, and/or the pharmaceutical composition of any one of claims 56-58 to the subject.

77. The method of claim 76, wherein the cell proliferation disorder is cancer.


SEQUENCE LISTING (9 sequences)

SEQ ID NO: 1 - protein, 448 aa, synthetic construct
QVQLVQSGAEVKKPGASVKVSCKASGYTFTSYWMHWVRQAPGQGLEWMGYINPSSGYTKSNQKFKDRVTMTADTSTSTAYMELSSLRSEDTAVYYCGRWLLSAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIQMTQSPSSLSASVGDRVTITCKASQDINTYLSWFQQKPGKSPKSLIYRSNILVDGVPSRFSGSGSGQDYTLTISSLQPEDFAIYYCLQYDDFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 444 aa, synthetic construct
QVQLVQSGAEVKKPGASVKVSCKASGYTFTSYWMHWVRQAPGQGLEWMGYINPSSGYTKSNQKFKDRVTMTADTSTSTAYMELSSLRSEDTAVYYCGRWLLSAWFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 4 - protein, 451 aa, synthetic construct
QVQLEQSGPEVVKPGASVKVSCKASGYSFTSYWMHWVRQAPGQGLEWMGMIDPSDSESRLNQKFKDRVTMTVDTPTSTVYMELSSPRSEDTAVYYCARSGYHGTSYWYFDVWGQGLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 219 aa, synthetic construct
DIVMTQSPLSLPVTPGEPASISCRSSKSLLHSDGITYLYWYLQKPGQSPQLLIYQMSNLASGVPDRFSGSGSGTDFTLKISRVEAEDVGVYYCAQNLELPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 473 aa, synthetic construct
MEFGLSWVFLVAILKGVQCEVQLVESGGGLVQPGGSLRLSCAASGFDFSRYWMSWVRQAPGKGLEWIGEINPDSSTIVYTPSLKDKFIISRDNAKNTLYLQMNSLRAEDTALYYCARRGSHYYGYRTGYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 233 aa, synthetic construct
MVSPLQFLRLLLLWVPASRGDVVMTQSPAFLSVTPGEKVTMTCSASSSVSYMYWHQQKPDQAPKLLIYDTSNLASGVPVRFSGSGSGTDFTFTISRMEAEDAATYYCQQWSSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 448 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 218 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC